SEQUENCE LISTING (141 sequences)

SEQ ID NO: 1 - DNA, 1894 bp, Homo sapiens
atggagccctcctggcttcaggaactcatggctcaccccttcttgctgctgatcctcctctgcatgtctctgctgctgtttcaggtaatcaggttgtaccagaggaggagatggatgatcagagccctgcacctgtttcctgcaccccctgcccactggttctatggccacaaggagttttacccagtaaaggagtttgaggtgtatcataagctgatggaaaaatacccatgtgctgttcccttgtgggttggaccctttacgatgttcttcagtgtccatgacccagactatgccaagattctcctgaaaagacaagatcccaaaagtgctgttagccacaaaatccttgaatcctgggttggtcgaggacttgtgaccctggatggttctaaatggaaaaagcaccgccagattgtgaaacctggcttcaacatcagcattctgaaaatattcatcaccatgatgtctgagagtgttcggatgatgctgaacaaatgggaggaacgcattgcccaaaactcacgtctggagctctttcaacatgtctccctgatgaccctggacagcatcatgaagtgtgccttcagccaccagggcagcatccagttggacagtaccctggactcatacctgaaagcagtgttcaaccttagcaaaatctccaaccagcgcatgaacaattttctacatcacaacgacctggttttcaaattcagctctcaaggccaaatcttttctaaatttaaccaagaacttcatcagttcacagagaaagtaatccaggaccggaaggagtctcttaaggataagctaaaacaagatactactcagaaaaggcgctgggattttctggacatacttttgagtgccaaaagcgaaaacaccaaagatttctctgaagcagatctccaggctgaagtgaaaacgttcatgtttgcaggacatgacaccacatccagtgctatctcctggatcctttactgcttggcaaagtaccctgagcatcagcagagatgccgagatgaaatcagggaactcctaggggatgggtcttctattacctgggaacacctgagccagatgccttacaccacgatgtgcatcaaggaatgcctccgcctctacgcaccggtagtaaacatatcccggttactcgacaaacccatcacctttccagatggacgctccttacctgcaggaataactgtgtttatcaatatttgggctcttcaccacaacccctatttctgggaagaccctcaggtctttaaccccttgagattctccagggaaaattctgaaaaaatacatccctatgccttcataccattctcagctggattaaggaactgcattgggcagcattttgccataattgagtgtaaagtggcagtggcattaactctgctccgcttcaagctggctccagaccactcaaggcctccccagcctgttcgtcaagttgtcctcaagtccaagaatggaatccatgtgtttgcaaaaaaagtttgctaattttaagtcctttcgtataagaattaatgagacaattttcctaccaaaggaagaacaaaaggataaatataatacaaaatatatgtatatggttgtttgacaaattatataacttaggatacttctgactggttttgacatccattaacagtaattttaatttctttgctgtatctggtgaaacccacaaaaacacctgaaaaaactcaagctgacttccactgcgaagggaaattattggtttgtgtaactagtggtagagtggctttcaagcatagtttgatcaaaactccactcagtatctgcattacttttatctctgcaaatatctgcatgatagctttattctcagttatctttccccaataataaaaaa

SEQ ID NO: 2 - protein, 505 aa, Homo sapiens
MEPSWLQELMAHPFLLLILLCMSLLLFQVIRLYQRRRWMIRALHLFPAPPAHWFYGHKEFYPVKEFEVYHKLMEKYPCAVPLWVGPFTMFFSVHDPDYAKILLKRQDPKSAVSHKILESWVGRGLVTLDGSKWKKHRQIVKPGFNISILKIFITMMSESVRMMLNKWEERIAQNSRLELFQHVSLMTLDSIMKCAFSHQGSIQLDSTLDSYLKAVFNLSKISNQRMNNFLHHNDLVFKFSSQGQIFSKFNQELHQFTEKVIQDRKESLKDKLKQDTTQKRRWDFLDILLSAKSENTKDFSEADLQAEVKTFMFAGHDTTSSAISWILYCLAKYPEHQQRCRDEIRELLGDGSSITWEHLSQMPYTTMCIKECLRLYAPVVNISRLLDKPITFPDGRSLPAGITVFINIWALHHNPYFWEDPQVFNPLRFSRENSEKIHPYAFIPFSAGLRNCIGQHFAIIECKVAVALTLLRFKLAPDHSRPPQPVRQVVLKSKNGIHVFAKKVC

SEQ ID NO: 3 - DNA, 4458 bp, Homo sapiens
ctagtctataccagcaacgactcctacatcgtccactctggggatcttagaaagatccataaagctgcctcccggggacaagtccggaagctggagaagatgacaaagaggaagaagaccatcaaccttaatatacaagacgcccagaagaggactgctctacactgggcctgtgtcaatggccatgaggaagtagtaacatttctggtagacagaaagtgccagcttgacgtccttgatggcgaacacaggacacctctgatgaaggctctacaatgccatcaggaggcttgtgcaaatattctgatagattctggtgccgatataaatctcgtagatgtgtatggcaacatggctctccattatgctgtttatagtgagattttgtcagtggtggcaaaactgctgtcccatggtgcagtcatcgaagtgcacaacaaggctagcctcacaccacttttactatccataacgaaaagaagtgagcaaattgtggaatttttgctgataaaaaatgcaaatgcgaatgcagttaataagtataaatgcacagccctcatgcttgctgtatgtcatggatcatcagagatagttggcatgcttcttcagcaaaatgttgacgtctttgctgcagatatatgtggagtaactgcagaacattatgctgttacttgtggatttcatcacattcatgaacaaattatggaatatatacgaaaattatctaaaaatcatcaaaataccaatccagaaggaacatctgcaggaacacctgatgaggctgcacccttggcggaaagaacacctgacacagctgaaagcttggtggaaaaaacacctgatgaggctgcacccttggtggaaagaacacctgacacggctgaaagcttggtggaaaaaacacctgatgaggctgcatccttggtggagggaacatctgacaaaattcaatgtttggagaaagcgacatctggaaagttcgaacagtcagcagaagaaacacctagggaaattacgagtcctgcaaaagaaacatctgagaaatttacgtggccagcaaaaggaagacctaggaagatcgcatgggagaaaaaagaagacacacctagggaaattatgagtcccgcaaaagaaacatctgagaaatttacgtgggcagcaaaaggaagacctaggaagatcgcatgggagaaaaaagaaacacctgtaaagactggatgcgtggcaagagtaacatctaataaaactaaagttttggaaaaaggaagatctaagatgattgcatgtcctacaaaagaatcatctacaaaagcaagtgccaatgatcagaggttcccatcagaatccaaacaagaggaagatgaagaatattcttgtgattctcggagtctctttgagagttctgcaaagattcaagtgtgtatacctgagtctatatatcaaaaagtaatggagataaatagagaagtagaagagcctcctaagaagccatctgccttcaagcctgccattgaaatgcaaaactctgttccaaataaagcctttgaattgaagaatgaacaaacattgagagcagatccgatgttcccaccagaatccaaacaaaaggactatgaagaaaattcttgggattctgagagtctctgtgagactgtttcacagaaggatgtgtgtttacccaaggctacacatcaaaaagaaatagataaaataaatggaaaattagaagagtctcctaataaagatggtcttctgaaggctacctgcggaatgaaagtttctattccaactaaagccttagaattgaaggacatgcaaactttcaaagcggagcctccggggaagccatctgccttcgagcctgccactgaaatgcaaaagtctgtcccaaataaagccttggaattgaaaaatgaacaaacatggagagcagatgagatactcccatcagaatccaaacaaaaggactatgaagaaaattcttgggatactgagagtctctgtgagactgtttcacagaaggatgtgtgtttacccaaggctgcgcatcaaaaagaaatagataaaataaatggaaaattagaagggtctcctgttaaagatggtcttctgaaggctaactgcggaatgaaagtttctattccaactaaagccttagaattgatggacatgcaaactttcaaagcagagcctcccgagaagccatctgccttcgagcctgccattgaaatgcaaaagtctgttccaaataaagccttggaattgaagaatgaacaaacattgagagcagatgagatactcccatcagaatccaaacaaaaggactatgaagaaagttcttgggattctgagagtctctgtgagactgtttcacagaaggatgtgtgtttacccaaggctacacatcaaaaagaaatagataaaataaatggaaaattagaagagtctcctgataatgatggttttctgaaggctccctgcagaatgaaagtttctattccaactaaagccttagaattgatggacatgcaaactttcaaagcagagcctcccgagaagccatctgccttcgagcctgccattgaaatgcaaaagtctgttccaaataaagccttggaattgaagaatgaacaaacattgagagcagatcagatgttcccttcagaatcaaaacaaaagaaggttgaagaaaattcttgggattctgagagtctccgtgagactgtttcacagaaggatgtgtgtgtacccaaggctacacatcaaaaagaaatggataaaataagtggaaaattagaagattcaactagcctatcaaaaatcttggatacagttcattcttgtgaaagagcaagggaacttcaaaaagatcactgtgaacaacgtacaggaaaaatggaacaaatgaaaaagaagttttgtgtactgaaaaagaaactgtcagaagcaaaagaaataaaatcacagttagagaaccaaaaagttaaatgggaacaagagctctgcagtgtgagattgactttaaaccaagaagaagagaagagaagaaatgccgatatattaaatgaaaaaattagggaagaattaggaagaatcgaagagcagcataggaaagagttagaagtgaaacaacaacttgaacaggctctcagaatacaagatatagaattgaagagtgtagaaagtaatttgaatcaggtttctcacactcatgaaaatgaaaattatctcttacatgaaaattgcatgttgaaaaaggaaattgccatgctaaaactggaaatagccacactgaaacaccaataccaggaaaaggaaaataaatactttgaggacattaagattttaaaagaaaagaatgctgaacttcagatgaccctaaaactgaaagaggaatcattaactaaaagggcatctcaatatagtgggcagcttaaagttctgatagctgagaacacaatgctcacttctaaattgaaggaaaaacaagacaaagaaatactagaggcagaaattgaatcacaccatcctagactggcttctgctgtacaagaccatgatcaaattgtgacatcaagaaaaagtcaagaacctgctttccacattgcaggagatgcttgtttgcaaagaaaaatgaatgttgatgtgagtagtacgatatataacaatgaggtgctccatcaaccactttctgaagctcaaaggaaatccaaaagcctaaaaattaatctcaattatgcaggagatgctctaagagaaaatacattggtttcagaacatgcacaaagagaccaacgtgaaacacagtgtcaaatgaaggaagctgaacacatgtatcaaaacgaacaagataatgtgaacaaacacactgaacagcaggagtctctagatcagaaattatttcaactacaaagcaaaaatatgtggcttcaacagcaattagttcatgcacataagaaagctgacaacaaaagcaagataacaattgatattcattttcttgagaggaaaatgcaacatcatctcctaaaagagaaaaatgaggagatatttaattacaataaccatttaaaaaaccgtatatatcaatatgaaaaagagaaagcagaaacagaaaactcatgagagacaagcagtaagaaacttcttttggagaaacaacagaccagatctttactcacaactcatgctaggaggccagtcctagcatcaccttatgttgaaaatcttaccaatagtctgtgtcaacagaatacttattttagaagaaaaattcatgatttcttcctgaagcctacagacataaaataacagtgtgaagaattacttgttcacgaattgcataaagctgcacaggattcccatctaccctgatgatgcagcagacatcattcaatccaaccagaatctcgctctgcactccagcctaggtgacagagtgagactccacctcggaaa

SEQ ID NO: 4 - protein, 1341 aa, Homo sapiens
MTKRKKTINLNIQDAQKRTALHWACVNGHEEVVTFLVDRKCQLDVLDGEHRTPLMKALQCHQEACANILIDSGADINLVDVYGNMALHYAVYSEILSVVAKLLSHGAVIEVHNKASLTPLLLSITKRSEQIVEFLLIKNANANAVNKYKCTALMLAVCHGSSEIVGMLLQQNVDVFAADICGVTAEHYAVTCGFHHIHEQIMEYIRKLSKNHQNTNPEGTSAGTPDEAAPLAERTPDTAESLVEKTPDEAAPLVERTPDTAESLVEKTPDEAASLVEGTSDKIQCLEKATSGKFEQSAEETPREITSPAKETSEKFTWPAKGRPRKIAWEKKEDTPREIMSPAKETSEKFTWAAKGRPRKIAWEKKETPVKTGCVARVTSNKTKVLEKGRSKMIACPTKESSTKASANDQRFPSESKQEEDEEYSCDSRSLFESSAKIQVCIPESIYQKVMEINREVEEPPKKPSAFKPAIEMQNSVPNKAFELKNEQTLRADPMFPPESKQKDYEENSWDSESLCETVSQKDVCLPKATHQKEIDKINGKLEESPNKDGLLKATCGMKVSIPTKALELKDMQTFKAEPPGKPSAFEPATEMQKSVPNKALELKNEQTWRADEILPSESKQKDYEENSWDTESLCETVSQKDVCLPKAAHQKEIDKINGKLEGSPVKDGLLKANCGMKVSIPTKALELMDMQTFKAEPPEKPSAFEPAIEMQKSVPNKALELKNEQTLRADEILPSESKQKDYEESSWDSESLCETVSQKDVCLPKATHQKEIDKINGKLEESPDNDGFLKAPCRMKVSIPTKALELMDMQTFKAEPPEKPSAFEPAIEMQKSVPNKALELKNEQTLRADQMFPSESKQKKVEENSWDSESLRETVSQKDVCVPKATHQKEMDKISGKLEDSTSLSKILDTVHSCERARELQKDHCEQRTGKMEQMKKKFCVLKKKLSEAKEIKSQLENQKVKWEQELCSVRLTLNQEEEKRRNADILNEKIREELGRIEEQHRKELEVKQQLEQALRIQDIELKSVESNLNQVSHTHENENYLLHENCMLKKEIAMLKLEIATLKHQYQEKENKYFEDIKILKEKNAELQMTLKLKEESLTKRASQYSGQLKVLIAENTMLTSKLKEKQDKEILEAEIESHHPRLASAVQDHDQIVTSRKSQEPAFHIAGDACLQRKMNVDVSSTIYNNEVLHQPLSEAQRKSKSLKINLNYAGDALRENTLVSEHAQRDQRETQCQMKEAEHMYQNEQDNVNKHTEQQESLDQKLFQLQSKNMWLQQQLVHAHKKADNKSKITIDIHFLERKMQHHLLKEKNEEIFNYNNHLKNRIYQYEKEKAETENS

SEQ ID NO: 5 - DNA, 2032 bp, Homo sapiens
cgcggggcgcggagtcggcggggcctcgcgggacgcgggcagtgcggagaccgcggcgctgaggacgcgggagccgggagcgcacgcgcggggtggagttcagcctactctttcttagatgtgaaaggaaaggaagatcatttcatgccttgttgataaaggttcagacttctgctgattcataaccatttggctctgagctatgacaagagaggaaacaaaaagttaaacttacaagcctgccataagtgagaagcaaacttccttgataacatgcttttgcgaagtgcaggaaaattaaatgtgggcaccaagaaagaggatggtgagagtacagcccccaccccccgtccaaaggtcttgcgttgtaaatgccaccaccattgtccagaagactcagtcaacaatatttgcagcacagacggatattgtttcacgatgatagaagaggatgactctgggttgcctgtggtcacttctggttgcctaggactagaaggctcagattttcagtgtcgggacactcccattcctcatcaaagaagatcaattgaatgctgcacagaaaggaacgaatgtaataaagacctacaccctacactgcctccattgaaaaacagagattttgttgatggacctatacaccacagggctttacttatatctgtgactgtctgtagtttgctcttggtccttatcatattattttgttacttccggtataaaagacaagaaaccagacctcgatacagcattgggttagaacaggatgaaacttacattcctcctggagaatccctgagagacttaattgagcagtctcagagctcaggaagtggatcaggcctccctctgctggtccaaaggactatagctaagcagattcagatggtgaaacagattggaaaaggtcgctatggggaagtttggatgggaaagtggcgtggcgaaaaggtagctgtgaaagtgttcttcaccacagaggaagccagctggttcagagagacagaaatatatcagacagtgttgatgaggcatgaaaacattttgggtttcattgctgcagatatcaaagggacagggtcctggacccagttgtacctaatcacagactatcatgaaaatggttccctttatgattatctgaagtccaccaccctagacgctaaatcaatgctgaagttagcctactcttctgtcagtggcttatgtcatttacacacagaaatctttagtactcaaggcaaaccagcaattgcccatcgagatctgaaaagtaaaaacattctggtgaagaaaaatggaacttgctgtattgctgacctgggcctggctgttaaatttattagtgatacaaatgaagttgacataccacctaacactcgagttggcaccaaacgctatatgcctccagaagtgttggacgagagcttgaacagaaatcacttccagtcttacatcatggctgacatgtatagttttggcctcatcctttgggaggttgctaggagatgtgtatcaggaggtatagtggaagaataccagcttccttatcatgacctagtgcccagtgacccctcttatgaggacatgagggagattgtgtgcatcaagaagttacgcccctcattcccaaaccggtggagcagtgatgagtgtctaaggcagatgggaaaactcatgacagaatgctgggctcacaatcctgcatcaaggctgacagccctgcgggttaagaaaacacttgccaaaatgtcagagtcccaggacattaaactctgataggagaggaaaagtaagcatctctgcagaaagccaacaggtactcttctgtttgtgggcagagcaaaagacatcaaataagcatccacagtacaagccttgaacatcgtcctgcttcccagtgggttcagacctcacctttcagggagcgacctgggcaaagacagagaagctcccagaaggagagattgatccgtgtctgtttgtaggcggagaaaccgttgggtaacttgttcaagatatgatgcat

SEQ ID NO: 6 - protein, 502 aa, Homo sapiens
MLLRSAGKLNVGTKKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHRALLISVTVCSLLLVLIILFCYFRYKRQETRPRYSIGLEQDETYIPPGESLRDLIEQSQSSGSGSGLPLLVQRTIAKQIQMVKQIGKGRYGEVWMGKWRGEKVAVKVFFTTEEASWFRETEIYQTVLMRHENILGFIAADIKGTGSWTQLYLITDYHENGSLYDYLKSTTLDAKSMLKLAYSSVSGLCHLHTEIFSTQGKPAIAHRDLKSKNILVKKNGTCCIADLGLAVKFISDTNEVDIPPNTRVGTKRYMPPEVLDESLNRNHFQSYIMADMYSFGLILWEVARRCVSGGIVEEYQLPYHDLVPSDPSYEDMREIVCIKKLRPSFPNRWSSDECLRQMGKLMTECWAHNPASRLTALRVKKTLAKMSESQDIKL

SEQ ID NO: 7 - DNA, 3238 bp, Homo sapiens
aactgagctaacaagaaatactagaaaaggaggaaggagaacattgctgcagcttggatctacaacctaagaaagcaagagtgatcaatctcagctctgttaaacatcttgtttacttactgcattcagcagcttgcaaatggttaactatatgcaaaaaagtcagcatagctgtgaagtatgccgtgaattttaattgagggaaaaagggacaattgcttcaggatgctctagtatgcactctgcttgaaatattttcaatgaaatgctcagtattctatctttgaccagaggttttaactttatgaagctatgggacttgacaaaaagtgatatttgagaagaaagtacgcagtggttggtgttttcttttttttaataaaggaattgaattactttgaacacctcttccagctgtgcattacagataacgtcaggaagagtctctgctttacagaatcggatttcatcacatgacaacatgaagctgtggattcatctcttttattcatctctccttgcctgtatatctttacactcccaaactccagtgctctcatccagaggctcttgtgattctctttgcaattgtgaggaaaaagatggcacaatgctaataaattgtgaagcaaaaggtatcaagatggtatctgaaataagtgtgccaccatcacgacctttccaactaagcttattaaataacggcttgacgatgcttcacacaaatgacttttctgggcttaccaatgctatttcaatacaccttggatttaacaatattgcagatattgagataggtgcatttaatggccttggcctcctgaaacaacttcatatcaatcacaattctttagaaattcttaaagaggatactttccatggactggaaaacctggaattcctgcaagcagataacaattttatcacagtgattgaaccaagtgcctttagcaagctcaacagactcaaagtgttaattttaaatgacaatgctattgagagtcttcctccaaacatcttccgatttgttcctttaacccatctagatcttcgtggaaatcaattacaaacattgccttatgttggttttctcgaacacattggccgaatattggatcttcagttggaggacaacaaatgggcctgcaattgtgacttattgcagttaaaaacttggttggagaacatgcctccacagtctataattggtgatgttgtctgcaacagccctccattttttaaaggaagtatactcagtagactaaagaaggaatctatttgccctactccaccagtgtatgaagaacatgaggatccttcaggatcattacatctggcagcaacatcttcaataaatgatagtcgcatgtcaactaagaccacgtccattctaaaactacccaccaaagcaccaggtttgataccttatattacaaagccatccactcaacttccaggaccttactgccctattccttgtaactgcaaagtcctatccccatcaggacttctaatacattgtcaggagcgcaacattgaaagcttatcagatctgagacctcctccgcaaaatcctagaaagctcattctagcgggaaatattattcacagtttaatgaagtctgatctagtggaatatttcactttggaaatgcttcacttgggaaacaatcgtattgaagttcttgaagaaggatcgtttatgaacctaacgagattacaaaaactctatctaaatggtaaccacctgaccaaattaagtaaaggcatgttccttggtctccataatcttgaatacttatatcttgaatacaatgccattaaggaaatactgccaggaacctttaatccaatgcctaaacttaaagtcctgtatttaaataacaacctcctccaagttttaccaccacatattttttcaggggttcctctaactaaggtaaatcttaaaacaaaccagtttacccatctacctgtaagtaatattttggatgatcttgatttactaacccagattgaccttgaggataacccctgggactgctcctgtgacctggttggactgcagcaatggatacaaaagttaagcaagaacacagtgacagatgacatcctctgcacttcccccgggcatctcgacaaaaaggaattgaaagccctaaatagtgaaattctctgtccaggtttagtaaataacccatccatgccaacacagactagttaccttatggtcaccactcctgcaacaacaacaaatacggctgatactattttacgatctcttacggacgctgtgccactgtctgttctaatattgggacttctgattatgttcatcactattgttttctgtgctgcagggatagtggttcttgttcttcaccgcaggagaagatacaaaaagaaacaagtagatgagcaaatgagagacaacagtcctgtgcatcttcagtacagcatgtatggccataaaaccactcatcacactactgaaagaccctctgcctcactctatgaacagcacatggtgagccccatggttcatgtctatagaagtccatcctttggtccaaagcatctggaagaggaagaagagaggaatgagaaagaaggaagtgatgcaaaacatctccaaagaagtcttttggaacaggaaaatcattcaccactcacagggtcaaatatgaaatacaaaaccacgaaccaatcaacagaatttttatccttccaagatgccagctcattgtacagaaacattttagaaaaagaaagggaacttcagcaactgggaatcacagaatacctaaggaaaaacattgctcagctccagcctgatatggaggcacattatcctggagcccacgaagagctgaagttaatggaaacattaatgtactcacgtccaaggaaggtattagtggaacagacaaaaaatgagtattttgaacttaaagctaatttacatgctgaacctgactatttagaagtcctggagcagcaaacatagatggagagtttgagggctttcgcagaaatgctgtgattctgttttaagtccataccttgtaaataagtgccttacgtgagtgtgtcatcaatcagaacctaagcacagcagtaaactatggggaaaaaaaaagaagaagaaaagaaactcagggatcactgggagaagccatggcattatcttcaggcaatttagtctgtcccaaataaaatcaatccttgcatgtaaatc

SEQ ID NO: 8 - protein, 841 aa, Homo sapiens
MKLWIHLFYSSLLACISLHSQTPVLSSRGSCDSLCNCEEKDGTMLINCEAKGIKMVSEISVPPSRPFQLSLLNNGLTMLHTNDFSGLTNAISIHLGFNNIADIEIGAFNGLGLLKQLHINHNSLEILKEDTFHGLENLEFLQADNNFITVIEPSAFSKLNRLKVLILNDNAIESLPPNIFRFVPLTHLDLRGNQLQTLPYVGFLEHIGRILDLQLEDNKWACNCDLLQLKTWLENMPPQSIIGDVVCNSPPFFKGSILSRLKKESICPTPPVYEEHEDPSGSLHLAATSSINDSRMSTKTTSILKLPTKAPGLIPYITKPSTQLPGPYCPIPCNCKVLSPSGLLIHCQERNIESLSDLRPPPQNPRKLILAGNIIHSLMKSDLVEYFTLEMLHLGNNRIEVLEEGSFMNLTRLQKLYLNGNHLTKLSKGMFLGLHNLEYLYLEYNAIKEILPGTFNPMPKLKVLYLNNNLLQVLPPHIFSGVPLTKVNLKTNQFTHLPVSNILDDLDLLTQIDLEDNPWDCSCDLVGLQQWIQKLSKNTVTDDILCTSPGHLDKKELKALNSEILCPGLVNNPSMPTQTSYLMVTTPATTTNTADTILRSLTDAVPLSVLILGLLIMFITIVFCAAGIVVLVLHRRRRYKKKQVDEQMRDNSPVHLQYSMYGHKTTHHTTERPSASLYEQHMVSPMVHVYRSPSFGPKHLEEEEERNEKEGSDAKHLQRSLLEQENHSPLTGSNMKYKTTNQSTEFLSFQDASSLYRNILEKERELQQLGITEYLRKNIAQLQPDMEAHYPGAHEELKLMETLMYSRPRKVLVEQTKNEYFELKANLHAEPDYLEVLEQQT

SEQ ID NO: 9 - DNA, 5062 bp, Homo sapiens
cactaacgctcttcctagtccccgggccaactcggacagtttgctcatttattgcaacggtcaaggctggcttgtgccagaacggcgcgcgcgcgacgcacgcacacacacggggggaaacttttttaaaaatgaaaggctagaagagctcagcggcggcgcgggccgtgcgcgagggctccggagctgactcgccgaggcaggaaatccctccggtcgcgacgcccggccccgctcggcgcccgcgtgggatggtgcagcgctcgccgccgggcccgagagctgctgcactgaaggccggcgacgatggcagcgcgcccgctgcccgtgtcccccgcccgcgccctcctgctcgccctggccggtgctctgctcgcgccctgcgaggcccgaggggtgagcttatggaacgaaggaagagctgatgaagttgtcagtgcctctgttcggagtggggacctctggatcccagtgaagagcttcgactccaagaatcatccagaagtgctgaatattcgactacaacgggaaagcaaagaactgatcataaatctggaaagaaatgaaggtctcattgccagcagtttcacggaaacccactatctgcaagacggtactgatgtctccctcgctcgaaattacacggtaattctgggtcactgttactaccatggacatgtacggggatattctgattcagcagtcagtctcagcacgtgttctggtctcaggggacttattgtgtttgaaaatgaaagctatgtcttagaaccaatgaaaagtgcaaccaacagatacaaactcttcccagcgaagaagctgaaaagcgtccggggatcatgtggatcacatcacaacacaccaaacctcgctgcaaagaatgtgtttccaccaccctctcagacatgggcaagaaggcataaaagagagaccctcaaggcaactaagtatgtggagctggtgatcgtggcagacaaccgagagtttcagaggcaaggaaaagatctggaaaaagttaagcagcgattaatagagattgctaatcacgttgacaagttttacagaccactgaacattcggatcgtgttggtaggcgtggaagtgtggaatgacatggacaaatgctctgtaagtcaggacccattcaccagcctccatgaatttctggactggaggaagatgaagcttctacctcgcaaatcccatgacaatgcgcagcttgtcagtggggtttatttccaagggaccaccatcggcatggccccaatcatgagcatgtgcacggcagaccagtctgggggaattgtcatggaccattcagacaatccccttggtgcagccgtgaccctggcacatgagctgggccacaatttcgggatgaatcatgacacactggacaggggctgtagctgtcaaatggcggttgagaaaggaggctgcatcatgaacgcttccaccgggtacccatttcccatggtgttcagcagttgcagcaggaaggacttggagaccagcctggagaaaggaatgggggtgtgcctgtttaacctgccggaagtcagggagtctttcgggggccagaagtgtgggaacagatttgtggaagaaggagaggagtgtgactgtggggagccagaggaatgtatgaatcgctgctgcaatgccaccacctgtaccctgaagccggacgctgtgtgcgcacatgggctgtgctgtgaagactgccagctgaagcctgcaggaacagcgtgcagggactccagcaactcctgtgacctcccagagttctgcacaggggccagccctcactgcccagccaacgtgtacctgcacgatgggcactcatgtcaggatgtggacggctactgctacaatggcatctgccagactcacgagcagcagtgtgtcacactctggggaccaggtgctaaacctgcccctgggatctgctttgagagagtcaattctgcaggtgatccttatggcaactgtggcaaagtctcgaagagttcctttgccaaatgcgagatgagagatgctaaatgtggaaaaatccagtgtcaaggaggtgccagccggccagtcattggtaccaatgccgtttccatagaaacaaacatccccctgcagcaaggaggccggattctgtgccgggggacccacgtgtacttgggcgatgacatgccggacccagggcttgtgcttgcaggcacaaagtgtgcagatggaaaaatctgcctgaatcgtcaatgtcaaaatattagtgtctttggggttcacgagtgtgcaatgcagtgccacggcagaggggtgtgcaacaacaggaagaactgccactgcgaggcccactgggcacctcccttctgtgacaagtttggctttggaggaagcacagacagcggccccatccggcaagcagataaccaaggtttaaccataggaattctggtgaccatcctgtgtcttcttgctgccggatttgtggtttatctcaaaaggaagaccttgatacgactgctgtttacaaataagaagaccaccattgaaaaactaaggtgtgtgcgcccttcccggccaccccgtggcttccaaccctgtcaggctcacctcggccaccttggaaaaggcctgatgaggaagccgccagattcctacccaccgaaggacaatcccaggagattgctgcagtgtcagaatgttgacatcagcagacccctcaacggcctgaatgtccctcagccccagtcaactcagcgagtgcttcctcccctccaccgggccccacgtgcacctagcgtccctgccagacccctgccagccaagcctgcacttaggcaggcccaggggacctgtaagccaaacccccctcagaagcctctgcctgcagatcctctggccagaacaactcggctcactcatgccttggccaggaccccaggacaatgggagactgggctccgcctggcacccctcagacctgctccacaatatccacaccaagtgcccagatccacccacaccgcctatattaagtgagaagccgacaccttttttcaacagtgaagacagaagtttgcactatctttcagctccagttggagttttttgtaccaacttttaggattttttttaatgtttaaaacatcattactataagaactttgagctactgccgtcagtgctgtgctgtgctatggtgctctgtctacttgcacaggtacttgtaaattattaatttatgcagaatgttgattacagtgcagtgcgctgtagtaggcatttttaccatcactgagttttccatggcaggaaggcttgttgtgcttttagtattttagtgaacttgaaatatcctgcttgatgggattctggacaggatgtgtttgctttctgatcaaggccttattggaaagcagtcccccaactacccccagctgtgcttatggtaccagatgcagctcaagagatcccaagtagaatctcagttgattttctggattccccatctcaggccagagccaaggggcttcaggtccaggctgtgtttggctttcagggaggccctgtgccccttgacaactggcaggcaggctcccagggacacctgggagaaatctggcttctggccaggaagctttggtgagaacctgggttgcagacaggaatcttaaggtgtagccacaccaggatagagactggaacactagacaagccagaacttgaccctgagctgaccagccgtgagcatgtttggaaggggtctgtagtgtcactcaaggcggtgcttgatagaaatgccaagcacttctttttctcgctgtcctttctagagcactgccaccagtaggttatttagcttgggaaaggtggtgtttctgtaagaaacctactgcccaggcactgcaaaccgccacctccctatactgcttggagctgagcaaatcaccacaaactgtaatacaatgatcctgtattcagacagatgaggactttccatgggaccacaactattttcagatgtgaaccattaaccagatctagtcaatcaagtctgtttactgcaaggttcaacttattaacaattaggcagactctttatgcttgcaaaaactacaaccaatggaatgtgatgttcatgggtatagttcatgtctgctatcattattcgtagatattggacaaagaaccttctctatggggcatcctctttttccaacttggctgcaggaatctttaaaagatgcttttaacagagtctgaacctatttcttaaacacttgcaacctacctgttgagcatcacagaatgtgataaggaaatcaacttgcttatcaacttcctaaatattatgagatgtggcttgggcagcatccccttgaactcttcactcttcaaatgcctgactagggagccatgtttcacaaggtctttaaagtgactaatggcatgagaaatacaaaaatactcagataaggtaaaatgccatgatgcctctgtcttctggactggttttcacattagaagacaattgacaacagttacataattcactctgagtgttttatgagaaagccttcttttggggtcaacagttttcctatgctttgaaacagaaaaatatgtaccaagaatcttggtttgccttccagaaaacaaaactgcatttcactttcccggtgttccccactgtatctaggcaacatagtattcatgactatggataaactaaacacgtgacacaaacacacacaaaagggaacccagctctaatacattccaactcgtatagcatgcatctgtttattctatagttattaagttctttaaaatgtaaagccatgctggaaaataatactgctgagatacatacagaattactgtaactgattacacttggtaattgtactaaagccaaacatatatatactattaaaaaggtttacagaattttatggtgcattacgtgggcattgtctttttagatgcccaaatccttagatctggcatgttagcccttcctccaattataagaggatatgaaccaaaaaaaaaaaaaaaaaaa

SEQ ID NO: 10 - protein, 909 aa, Homo sapiens
MAARPLPVSPARALLLALAGALLAPCEARGVSLWNEGRADEVVSASVRSGDLWIPVKSFDSKNHPEVLNIRLQRESKELIINLERNEGLIASSFTETHYLQDGTDVSLARNYTVILGHCYYHGHVRGYSDSAVSLSTCSGLRGLIVFENESYVLEPMKSATNRYKLFPAKKLKSVRGSCGSHHNTPNLAAKNVFPPPSQTWARRHKRETLKATKYVELVIVADNREFQRQGKDLEKVKQRLIEIANHVDKFYRPLNIRIVLVGVEVWNDMDKCSVSQDPFTSLHEFLDWRKMKLLPRKSHDNAQLVSGVYFQGTTIGMAPIMSMCTADQSGGIVMDHSDNPLGAAVTLAHELGHNFGMNHDTLDRGCSCQMAVEKGGCIMNASTGYPFPMVFSSCSRKDLETSLEKGMGVCLFNLPEVRESFGGQKCGNRFVEEGEECDCGEPEECMNRCCNATTCTLKPDAVCAHGLCCEDCQLKPAGTACRDSSNSCDLPEFCTGASPHCPANVYLHDGHSCQDVDGYCYNGICQTHEQQCVTLWGPGAKPAPGICFERVNSAGDPYGNCGKVSKSSFAKCEMRDAKCGKIQCQGGASRPVIGTNAVSIETNIPLQQGGRILCRGTHVYLGDDMPDPGLVLAGTKCADGKICLNRQCQNISVFGVHECAMQCHGRGVCNNRKNCHCEAHWAPPFCDKFGFGGSTDSGPIRQADNQGLTIGILVTILCLLAAGFVVYLKRKTLIRLLFTNKKTTIEKLRCVRPSRPPRGFQPCQAHLGHLGKGLMRKPPDSYPPKDNPRRLLQCQNVDISRPLNGLNVPQPQSTQRVLPPLHRAPRAPSVPARPLPAKPALRQAQGTCKPNPPQKPLPADPLARTTRLTHALARTPGQWETGLRLAPLRPAPQYPHQVPRSTHTAYIK

SEQ ID NO: 11 - DNA, 2514 bp, Homo sapiens
ggcacgagggctgagcgccggaggagcgtaggcagggcagcgctggcgccagcggcgacaggagccgcgcgaccggcaaaaatacacgggaggccgtcgccgaaaagagtccgcggtcctctctcgtaaacacactctcctccaccggcgcctccccctccgctctgcgcgccgcccggctgggcgcccgaggccgctccgactgctatgtgaccgcgaggctgcgggaggaaggggacagggaagaagaggctctcccgcgggagcccttgaggaccaagtttgcggccacttctgcaggcgtcccttcttagctctcgcctgcccctttctgcagcctaggcggcccaggttctcttctcttcctcgcgcgcccagccgcctcggttcccggcgaccatggtgacgatggaggagctgcgggagatggactgcagtgtgctcaaaaggctgatgaaccgggacgagaatggcggcggcgcgggcggcagcggcagccacggcaccctggggctgccgagcggcggcaagtgcctgctgctggactgcagaccgttcctggcgcacagcgcgggctacatcctaggttcggtcaacgtgcgctgtaacaccatcgtgcggcggcgggctaagggctccgtgagcctggagcagatcctgcccgccgaggaggaggtacgcgcccgcttgcgctccggcctctactcggcggtcatcgtctacgacgagcgcagcccgcgcgccgagagcctccgcgaggacagcaccgtgtcgctggtggtgcaggcgctgcgccgcaacgccgagcgcaccgacatctgcctgctcaaaggcggctatgagaggttttcctccgagtacccagaattctgttctaaaaccaaggccctggcagccatcccacccccggttccccccagcgccacagagcccttggacctgggctgcagctcctgtgggaccccactacacgaccaggggggtcctgtggagatccttcccttcctctacctcggcagtgcctaccatgctgcccggagagacatgctggacgccctgggcatcacggctctgttgaatgtctcctcggactgcccaaaccactttgaaggacactatcagtacaagtgcatcccagtggaagataaccacaaggccgacatcagctcctggttcatggaagccatagagtacatcgatgccgtgaaggactgccgtgggcgcgtgctggtgcactgccaggcgggcatctcgcggtcggccaccatctgcctggcctacctgatgatgaagaaacgggtgaggctggaggaggccttcgagttcgttaagcagcgccgcagcatcatctcgcccaacttcagcttcatggggcagctgctgcagttcgagtcccaggtgctggccacgtcctgtgctgcggaggctgctagcccctcgggacccctgcgggagcggggcaagacccccgccacccccacctcgcagttcgtcttcagctttccggtctccgtgggcgtgcactcggcccccagcagcctgccctacctgcacagccccatcaccacctctcccagctgttagagccgccctgggggccccagaaccagagctggctcccagcaagggtaggacgggccgcatgcggcagaaagttgggactgagcagctgggagcaggcgaccgagctccttccccatcatttctccttggccaacgacgaggccagccagaatggcaataaggactccgaatacataataaaagcaaacagaacactccaacttagagcaataaccggtgccgcagcagccagggaagaccttggtttggtttatgtgtcagtttcacttttccgatagaaatttcttacctcatttttttaagcagtaaggcttgaagtgatgaaacccacagatcctagcaaatgtgcccaaccagctttactaaagggggaggaagggagggcaaagggatgagaagacaagtttcccagaagtgcctggttctgggtacttgtccctttgttgtcgttgttgtagttaaaggaatttcatttttaaaagaaatcttcgaaggtgtggttttcatttctcagtcaccaacagatgaataattatgcttaataataaagtatttattaagactttcttcagagtatgaaagtacaaaaagtctagttacagtggatttagaatatatttatgttgatgtcaaacagctgagcaccgtagcatgcagatgtcaaggcagttaggaagtaaatggtgtcttgtagatatgtgcaaggtagcatgatgagcaacttgagtttgttgccactgagaagcaggcgggttgggtgggaggaggaagaaagggaagaattaggtttgaattgctttttaaaaaaaaaagaaaagaaaaagacagcatctcactatgttgccaaggctcatcttgagaagcaggcgggttgggtgggaggaggaagaaagggaagaattaggtttgaattgcttttttaaaaaaaaa

SEQ ID NO: 12 - protein, 394 aa, Homo sapiens
MVTMEELREMDCSVLKRLMNRDENGGGAGGSGSHGTLGLPSGGKCLLLDCRPFLAHSAGYILGSVNVRCNTIVRRRAKGSVSLEQILPAEEEVRARLRSGLYSAVIVYDERSPRAESLREDSTVSLVVQALRRNAERTDICLLKGGYERFSSEYPEFCSKTKALAAIPPPVPPSATEPLDLGCSSCGTPLHDQGGPVEILPFLYLGSAYHAARRDMLDALGITALLNVSSDCPNHFEGHYQYKCIPVEDNHKADISSWFMEAIEYIDAVKDCRGRVLVHCQAGISRSATICLAYLMMKKRVRLEEAFEFVKQRRSIISPNFSFMGQLLQFESQVLATSCAAEAASPSGPLRERGKTPATPTSQFVFSFPVSVGVHSAPSSLPYLHSPITTSPSC

SEQ ID NO: 13 - DNA, 492 bp, Homo sapiens
agcgccttgccttctcttaggctttgaagcatttttgtctgtgctccctgatcttcaggtcaccaccatgaagttcttagcagtcctggtactcttgggagtttccatctttctggtctctgcccagaatccgacaacagctgctccagctgacacgtatccagctactggtcctgctgatgatgaagcccctgatgctgaaaccactgctgctgcaaccactgcgaccactgctgctcctaccactgcaaccaccgctgcttctaccactgctcgtaaagacattccagttttacccaaatgggttggggatctcccgaatggtagagtgtgtccctgagatggaatcagcttgagtcttctgcaattgggtcacaactattcatgcttcctgtgatttcatccaactacttaccttgcctacgatatcccctttatctctaatcagtttattttctttcaaataaaaaataactatgagcgagctaacat

SEQ ID NO: 14 - protein, 90 aa, Homo sapiens
MKFLAVLVLLGVSIFLVSAQNPTTAAPADTYPATGPADDEAPDAETTAAATTATTAAPTTATTAASTTARKDIPVLPKWVGDLPNGRVCP

SEQ ID NO: 15 - DNA, 1695 bp, Homo sapiens
gcttaagttgacctctgggtccggaatcgcgggcaaagatggcggcggccaggtgttggaggcctttgctacgcggtccgaggctttcattgcacaccgcggctaatgccgccgccacggctacagaaacgacctcccaagacgtcgcggcgacccccgtcgcgcggtacccgccgattgtggcctccatgacagccgacagcaaagctgcacggctgcggcggatcgagcgctggcaggcgacggtgcacgctgcggagtcggtagacgagaagctgcgaatcctcaccaagatgcagtttatgaagtacatggtttacccgcagaccttcgcactgaatgccgaccgctggtaccagtacttcaccaagaccgtgttcctgtcgggtctgccgccgcccccagcggagcccgagcccgagcccgaacccgaacctgaacctgcgctggacctcgcggcgctgcgtgcggtcgcctgcgactgcctgctgcaggagcacttctacctgcggcgcaggcggcgcgtgcaccgttacgaggagagcgaggtcatatctttgcccttcctggatcagctggtgtcaaccctcgtgggcctcctcagcccacacaacccggccctggccgctgccgccctcgattatagatgcccagttcatttttactgggtgcgtggtgaagaaattattcctcgtggtcatcgaagaggtcgaattgatgacttgcgataccagatagatgataaaccaaacaaccagattcgaatatccaagcaactcgcagagtttgtgccattggattattctgttcctatagaaatccccactataaaatgtaaaccagacaaacttccattattcaaacggcagtatgaaaaccacatatttgttggctcaaaaactgcagatccttgctgttacggtcacacccagtttcatctgttacctgacaaattaagaagggaaaggcttttgagacaaaactgtgctgatcagatagaagttgtttttagagctaatgctattgcaagcctttttgcttggactggagcacaagctatgtatcaaggattctggagtgaagcagatgttactcgaccttttgtctcccaggctgtgatcacagatggaaaatacttttcctttttctgctaccagctaaatactttggcactgactacacaagctgatcaaaataaccctcgtaaaaatatatgttggggtacacaaagtaagcctctttatgaaacaattgaggataatgatgtgaaaggttttaatgatgatgttctacttcagatagttcactttctactgaatagaccaaaagaagaaaaatcacagctgttggaaaactgaaaaagcatatttgattgagaactgtgggaatatttaaattttactgaaggaacaataatgatgagatttgtaactgtcaactattaaatacattgatttttgagacaaatatttcttatgtcaacctgttattagatctcttactctgctcaaattcatcactgaaagatttaattttagttaccttttgttgatttaaaaataattgcatttgtatattgctaactgataagacaaattgagttattgagctattaaatgcacattttaatataaatgcagaaatcccaaataaaatgctaacatactgaattcagtaattaaaagaacccactgc

SEQ ID NO: 16 - protein, 439 aa, Homo sapiens
MAAARCWRPLLRGPRLSLHTAANAAATATETTSQDVAATPVARYPPIVASMTADSKAARLRRIERWQATVHAAESVDEKLRILTKMQFMKYMVYPQTFALNADRWYQYFTKTVFLSGLPPPPAEPEPEPEPEPEPALDLAALRAVACDCLLQEHFYLRRRRRVHRYEESEVISLPFLDQLVSTLVGLLSPHNPALAAAALDYRCPVHFYWVRGEEIIPRGHRRGRIDDLRYQIDDKPNNQIRISKQLAEFVPLDYSVPIEIPTIKCKPDKLPLFKRQYENHIFVGSKTADPCCYGHTQFHLLPDKLRRERLLRQNCADQIEVVFRANAIASLFAWTGAQAMYQGFWSEADVTRPFVSQAVITDGKYFSFFCYQLNTLALTTQADQNNPRKNICWGTQSKPLYETIEDNDVKGFNDDVLLQIVHFLLNRPKEEKSQLLEN

SEQ ID NO: 17 - DNA, 4151 bp, Homo sapiens
gcatgagcctggactgcaccagccatatcgcgctgggtgccgcttcgccagcgcccgaggaaacttacgatcatctttcggaagtcccggtcacgcgggagcagttaaaccactatcggaatgtggctcaaaatgctcgaagtgaacttgcagcaactttggtcaaatttgaatgtgctcagtctgagcttcaagacctccgatccaagatgctttctaaagaagtctcctgtcaagaactgaaagctgaaatggagagctacaaggaaaacaatgccagaaaatcatctctccttacctctttgagagacagagttcaggaactagaagaagaatcagcagcactttccacttctaaaatcagaacagaaatcacagctcacgctgcaatcaaggagaaccaggaattaaagaagaaagttgtagagttaaatgaaaaattacaaaagtgttcaaaagaaaatgaggagaataagaaacaagtttcaaagaattgcaggaaacatgaggaatttctgactcaactgcgtgactgcttggatccagatgagaggaatgacaaggcatcagatgaagatttaattttaaagcttagagacctgcgcaaagaaaatgaattcgtgaaaggacaaattgttattcttgaagagactataaatgtccatgagatggaagcaaaagctagcagagaaacgatcatgaggctggcttcagaagtcaacagagagcagaaaaaagctgcctcctgtactgaagagaaagagaagctgaaccaggacctgctcagtgctgtagaagcaaaagaagctcttgaaagggaagttaagatcttccaagaaaggctgcttgctggccagcaggtctgggatgcctcaaagcaggaagtgagcctcctgaagaaaagctcttctgagttggagaagagtttgaaggccagtcaggatgcagtcacaacctcacaaagccagtactcctcatttagggagaaaatcgcagccctccttaggggcagattgagcatgactgggtccactgaggacaccattttggagaagattcgagaaatggacagccgggaagaaagcagggaccggatggtctcccagcttgaagcccaaatatctgagcttgttgaacagttgggaaaggagtctgggtttcaccagaaagctctccagagggcccagaaagcagagaatatgttggagactcttcagggtcagctgacacacctggaggcagagctggtttctggaggtgttttgcgagacaacttgaattttgagaaacaaaaatatcttaaatttctggatcagctttctcagaaaatgaagttggaccagatggctgccgaacttggctttgacacgcggctggacgtggttttagctcgaacagagcagctggttcgtcttgagagcaatgcagtcattgagaacaagaccattgcccacaatttgcagagaaagctaaagacacagaaagagagactggagagcaaagaattacacatgagcctcctccggcagaaaatagcccagctggaggaggagaagcaggcacgcacggccttggtggttgagagggacaacgcgcatcttaccatcaggaacttgcagaagaaggtggagaggctgcagaaagagctgaacacgtgtcgagacttgcacaccgagctcaaagccaaactggccgacaccaatgaactgaagattaaaactttggaacagactaaagccattgaagatctaaacaaatccagagaccaactggagaagatgaaggagaaagctgagaaaaagctcatgtctgtcaagtcagaactggataccacagaacatgaggctaaggagaataaagaaagggccagaaacatgatagaagtggtaaccagtgaaatgaagacactaaaaaaatctctggaagaagcagaaaagagagaaaagcagctggcagacttcagggaggtggtgtcgcagatgctaggcttgaacgtgaccagccttgctcttcctgattatgaaatcatcaagtgtcttgaaagattggtccattcacatcagcatcactttgttacctgtgcctgcctcaaagatgtgactactgggcaagagaggcacccacaaggccatttacagcttcttcattgaacactgtatctcttgagagaggtggccataagacatggcacacaattcccaatttcacaaattcctcatgtctttgagatttgatcagtttgtgaatattttatgctttgatgatatagtgagaatgcatcacttgcaaaaacgatctcaaaagtgtcagccttagataaacgtcagcattaaaaaacgccaaaaaaaaaaaaaaaaagcattttaggatccagaagaattccaccagattgcatgagttagattgggaaatgggagtgggagataatattgggaggtatctattttaagtcaggggctttactagccgatttagttctcacaataaccatgtggagaagctgtgacatttttaatttacaacctttctggggctcagacataaagttacctatccaaggttgcagttgggtagtggtgggaccaggatggacaactcattggccctgcctcaaaagccatacctcttctcctgctatgcagaatctgtttctcctgaatctctgtgatgctggtgggaattgtttgcatagaggaaggacaataaccctgccatcgtgagttaatgtccgggctggtcacagtggttcatgcctgtaatcccagcactttgggagtccaaggcaggcatatcatttgaggtcaggagtttaagaccagcctggctaacatagtgagaccctgtttctactaaaaatacaaaaataagccaggtgtggtggtgcatgactgtaatcccaactactcagcaggagaagcacttgaacccaggagacggaggcggcagtgagccaagattgtgccactgcactccagcctgggcgacagagtgaaactacatctcagaaaaaaaaaaaaaaaaaaaaaaaaaaagttaatgtccaaaaatgacagatttacaagtgtaagctatatgatttcttcaaaaagcaaaagcaatatacctaattcatttggatcaaacttacataggtctcaggtcctgtaagaaacttgcctgttctaactgttgctaccagattatatctggtggtaattgttaatgtttcagcagggctggtctcagtcctttaaaatggaaagctttatttggagcccaaccctataagatgaagaaatcctatatagtcttattcaccaatatatccaatacacccacagcaatggtacctttttaagatcaggattttattatgaattcctgtcactttctgttttccatttaaatttctattttacaaatttttcagggaatcatattcttaacttcactgagaaaaatgtgttactcttttggacaatttatcttatttctatcatataagatgtatttttttattgtccttaaaagaagctctagcatgaaattaaaggaaagggaaagaaatagatctggtgcacccgaacattaggagaaaatgaaaaatatacaaccaaccgttcgtgagtcatcaaaaagtcaaagtcagcctggccaacatggcaaaactccgtctctgcaaaaaatacaaaaatgagcccggtatgttggcatatgcctgtaatcccagctactcgagaggctgagacacgaaaattgcttgaacctgggaggcggaggttgcaatgagccgagatcgcgctactgcactccagcctgggcaacagagagagaccttgtctcaaaaaacaacaacaacaaaaagtcaaagtcataataagcaaattattggcttctttcttctagacyaaaagaaattaaagagatgaaacaatcaattgcaagggtcaaaactagattggatcttggtttgaatgaaaaaaagcataaaatattcttgcaataattgtaaaaatttgaatgtggactaagtcctagattatattaaaatatttttaattttttaagcttgacaaatgcactgattgttatactttaaataactaaaaatctgagaatccacagtgctacagacaataaatgataaaatgggaaaaaaaaaaaaaaaaaaa

SEQ ID NO: 18 - protein, 715 aa, Homo sapiens
MSLDCTSHIALGAASPAPEETYDHLSEVPVTREQLNHYRNVAQNARSELAATLVKFECAQSELQDLRSKMLSKEVSCQELKAEMESYKENNARKSSLLTSLRDRVQELEEESAALSTSKIRTEITAHAAIKENQELKKKVVELNEKLQKCSKENEENKKQVSKNCRKHEEFLTQLRDCLDPDERNDKASDEDLILKLRDLRKENEFVKGQIVILEETINVHEMEAKASRETIMRLASEVNREQKKAASCTEEKEKLNQDLLSAVEAKEALEREVKIFQERLLAGQQVWDASKQEVSLLKKSSSELEKSLKASQDAVTTSQSQYSSFREKIAALLRGRLSMTGSTEDTILEKIREMDSREESRDRMVSQLEAQISELVEQLGKESGFHQKALQRAQKAENMLETLQGQLTHLEAELVSGGVLRDNLNFEKQKYLKFLDQLSQKMKLDQMAAELGFDTRLDVVLARTEQLVRLESNAVIENKTIAHNLQRKLKTQKERLESKELHMSLLRQKIAQLEEEKQARTALVVERDNAHLTIRNLQKKVERLQKELNTCRDLHTELKAKLADTNELKIKTLEQTKAIEDLNKSRDQLEKMKEKAEKKLMSVKSELDTTEHEAKENKERARNMIEVVTSEMKTLKKSLEEAEKREKQLADFREVVSQMLGLNVTSLALPDYEIIKCLERLVHSHQHHFVTCACLKDVTTGQERHPQGHLQLLH

SEQ ID NO: 19 - DNA, 1615 bp, Homo sapiens
ctttgtataaggctcagctaaaagggaaattgagtgggtcaggtaccacggatactatacactctattgcatgattctcctgcctacatcagaagacgtttataagcctattttaaaggataccagttggaatctctcttttattaatcaccaagagaaccatgaacaagctgtttatcatttgactcatcatttaatcttgatttccagcttctcacacttgaaagaagacataatacatttctcacaggattctgggactattaactgaacttatgtgtgtaaaaggaattcatacaatgaaagcactagaaataattattatacttataaccattgtatttttacatgtttaaaatatagccataattagcctactcaaatccaagtgtaaaagtaaaatgatttgctttcgttttgttttccttgcttaggggatcatggacattgaagcatatcttgaaagaattggctataagaagtctaggaacaaattggacttggaaacattaactgacattcttcaacaccagatccgagctgttccctttgagaaccttaacatccattgtggggatgccatggacttaggcttagaggccatttttgatcaagttgtgagaagaaatcggggtggatggtgtctccaggtcaatcatcttctgtactgggctctgaccactattggttttgagaccacgatgttgggagggtatgtttacagcactccagccaaaaaatacagcactggcatgattcaccttctcctgcaggtgaccattgatggcaggaactacattgtcgatgctgggtttggacgctcataccagatgtggcagcctctggagttaatttctgggaaggatcagcctcaggtgccttgtgtcttccgtttgacggaagagaatggattctggtatctagaccaaatcagaagggaacagtacattccaaatgaagaatttcttcattctgatctcctagaagacagcaaataccgaaaaatctactcctttactcttaagcctcgaacaattgaagattttgagtctatgaatacatacctgcagacatctccatcatctgtgtttactagtaaatcattttgttccttgcagaccccagatggggttcactgtttggtgggcttcaccctcacccataggagattcaattataaggacaatacagatctaatagagttcaagactctgagtgaggaagaaatagaaaaagtgctgaaaaatatatttaatatttccttgcagagaaagcttgtgcccaaacatggtgatagattttttactatttagaataaggagtaaaacaatcttgtctatttgtcatccagctcaccagttatcaactgacgacctatcatgtatcttctgtacccttaccttattttgaagaaaatcctagacatcaaatcatttcacctataaaaatgtcatcatatataattaaacagctttttaaagaaacataaccacaaaccttttcaaataataataataataataataataaatgtattttaaagatggcctgtggttatcttggaaattggtgatttatgctagaaagcttttaatgttggtttattgttgaattc

SEQ ID NO: 20 - protein, 290 aa, Homo sapiens
MDIEAYLERIGYKKSRNKLDLETLTDILQHQIRAVPFENLNIHCGDAMDLGLEAIFDQVVRRNRGGWCLQVNHLLYWALTTIGFETTMLGGYVYSTPAKKYSTGMIHLLLQVTIDGRNYIVDAGFGRSYQMWQPLELISGKDQPQVPCVFRLTEENGFWYLDQIRREQYIPNEEFLHSDLLEDSKYRKIYSFTLKPRTIEDFESMNTYLQTSPSSVFTSKSFCSLQTPDGVHCLVGFTLTHRRFNYKDNTDLIEFKTLSEEEIEKVLKNIFNISLQRKLVPKHGDRFFTI

SEQ ID NO: 21 - DNA, 1837 bp, Homo sapiens
cggcacgagcaaaaaggaagagtgggaggaggaggggaagcggcgaaggaggaagaggaggaggaggaagaggggagcacaaaggatccaggtctcccgacgggaggttaataccaagaaccatgtgtgccgagcggctgggccagttcatgaccctggctttggtgttggccacctttgacccggcgcgggggaccgacgccaccaacccacccgagggtccccaagacaggagctcccagcagaaaggccgcctgtccctgcagaatacagcggagatccagcactgtttggtcaacgctggcgatgtggggtgtggcgtgtttgaatgtttcgagaacaactcttgtgagattcggggcttacatgggatttgcatgacttttctgcacaacgctggaaaatttgatgcccagggcaagtcattcatcaaagacgccttgaaatgtaaggcccacgctctgcggcacaggttcggctgcataagccggaagtgcccggccatcagggaaatggtgtcccagttgcagcgggaatgctacctcaagcacgacctgtgcgcggctgcccaggagaacacccgggtgatagtggagatgatccatttcaaggacttgctgctgcacgaaccctacgtggacctcgtgaacttgctgctgacctgtggggaggaggtgaaggaggccatcacccacagcgtgcaggttcagtgtgagcagaactggggaagcctgtgctccatcttgagcttctgcacctcggccatccagaagcctcccacggcgccccccgagcgccagccccaggtggacagaaccaagctctccagggcccaccacggggaagcaggacatcacctcccagagcccagcagtagggagactggccgaggtgccaagggtgagcgaggtagcaagagccacccaaacgcccatgcccgaggcagagtcgggggccttggggctcagggaccttccggaagcagcgagtgggaagacgaacagtctgagtattctgatatccggaggtgaaatgaaaggcctggccacgaaatctttcctccacgccgtccattttcttatctatggacattccaaaacatttaccattagagaggggggatgtcacacgcaggattctgtggggactgtggacttcatcgaggtgtgtgttcgcggaacggacaggtgagatggagacccctggggccgtggggtctcaggggtgcctggtgaattctgcacttacacgtactcaagggagcgcgcccgcgttatcctcgtacctttgtcttctttccatctgtggagtcagtgggtgtcggccgctctgttgtgggggaggtgaaccagggaggggcagggcaaggcagggcccccagagctgggccacacagtgggtgctgggcctcgccccgaagcttctggtgcagcagcctctggtgctgtctccgcggaagtcagggcggctggattccaggacaggagtgaatgtaaaaataaatatcgcttagaatgcaggagaagggtggagaggaggcaggggccgagggggtgcttggtgccaaactgaaattcagtttcttgtgtggggccttgcggttcagagctcttggcgagggtggagggaggagtgtcatttctatgtgtaatttctgagccattgtactgtctgggctgggggggacactgtccaagggagtggcccctatgagtttatattttaaccactgcttcaaatctcgatttcactttttttatttatccagttatatctacatatctgtcatctaaataaatggctttcaaacaaaaaaaaaaaaaaaaaaaaaaaaaaa

SEQ ID NO: 22 - protein, 302 aa, Homo sapiens
MCAERLGQFMTLALVLATFDPARGTDATNPPEGPQDRSSQQKGRLSLQNTAEIQHCLVNAGDVGCGVFECFENNSCEIRGLHGICMTFLHNAGKFDAQGKSFIKDALKCKAHALRHRFGCISRKCPAIREMVSQLQRECYLKHDLCAAAQENTRVIVEMIHFKDLLLHEPYVDLVNLLLTCGEEVKEAITHSVQVQCEQNWGSLCSILSFCTSAIQKPPTAPPERQPQVDRTKLSRAHHGEAGHHLPEPSSRETGRGAKGERGSKSHPNAHARGRVGGLGAQGPSGSSEWEDEQSEYSDIRR

SEQ ID NO: 23 - DNA, 2560 bp, Homo sapiens
gaattccggccagaagaaatctggcctcggaacacgccattctccgcgccgcttccaataaccactaacatccctaacgagcatccgagccgagggctctgctcggaaatcgtcctggcccaactcggcccttcgagctctcgaagattaccgcatctattttttttttcttttttttcttttcctagcgcagataaagtgagcccggaaagggaaggagggggcggggacaccattgccctgaaagaataaataagtaaataaacaaactggctcctcgccgcagctggacgcggtcggttgagtccaggttgggtcggacctgaacccctaaaagcggaaccgcctcccgccctcgccatcccggagctgagtcgccggcggcggtggctgctgccagacccggagtttcctctttcactggatggagctgaactttgggcggccagagcagcacagctgtccggggatcgctgcacgctgagctccctcggcaagacccagcggcggctcgggatttttttgggggggcggggaccagccccgcgccggcaccatgttcctggcgaccctgtacttcgcgctgccgctcttggacttgctcctgtcggccgaagtgagcggcggagaccgcctggattgcgtgaaagccagtgatcagtgcctgaaggagcagagctgcagcaccaagtaccgcacgctaaggcagtgcgtggcgggcaaggagaccaacttcagcctggcatccggcctggaggccaaggatgagtgccgcagcgccatggaggccctgaagcagaagtcgctctacaactgccgctgcaagcggggtatgaagaaggagaagaactgcctgcgcatttactggagcatgtaccagagcctgcagggaaatgatctgctggaggattccccatatgaaccagttaacagcagattgtcagatatattccgggtggtcccattcatatcagatgtttttcagcaagtggagcacattcccaaagggaacaactgcctggatgcagcgaaggcctgcaacctcgacgacatttgcaagaagtacaggtcggcgtacatcaccccgtgcaccaccagcgtgtccaacgatgtctgcaaccgccgcaagtgccacaaggccctccggcagttctttgacaaggtcccggccaagcacagctacggaatgctcttctgctcctgccgggacatcgcctgcacagagcggaggcgacagaccatcgtgcctgtgtgctcctatgaagagagggagaagcccaactgtttgaatttgcaggactcctgcaagacgaattacatctgcagatctcgccttgcggatttttttaccaactgccagccagagtcaaggtctgtcagcagctgtctaaaggaaaactacgctgactgcctcctcgcctactcggggcttattggcacagtcatgacccccaactacatagactccagtagcctcagtgtggccccatggtgtgactgcagcaacagtgggaacgacctagaagagtgcttgaaatttttgaatttcttcaaggacaatacatgtcttaaaaatgcaattcaagcctttggcaatggctccgatgtgaccgtgtggcagccagccttcccagtacagaccaccactgccactaccaccactgccctccgggttaagaacaagcccctggggccagcagggtctgagaatgaaattcccactcatgttttgccaccgtgtgcaaatttacaggcacagaagctgaaatccaatgtgtcgggcaatacacacctctgtatttccaatggtaattatgaaaaagaaggtctcggtgcttccagccacataaccacaaaatcaatggctgctcctccaagctgtggtctgagcccactgctggtcctggtggtaaccgctctgtccaccctattatctttaacagaaacatcatagctgcattaaaaaaatacaatatggacatgtaaaaagacaaaaaccaagttatctgtttcctgttctcttgtatagctgaaattccagtttaggagctcagttgagaaacagttccattcaactggaacatttttttttttccttttaagaaagcttcttgtgatccttcggggcttctgtgaaaaacctgatgcagtgctccatccaaactcagaaggctttgggatatgctgtattttaaagggacagtttgtaacttgggctgtaaagcaaactggggctgtgttttcgatgatgatgatcatcatgatcatgatgattttaacagttttacttctggcctttcctagctagagaaggagttaatatttctaaggtaactcccatatctcctttaatgacattgatttctaatgatataaatttcagcctacattgatgccaagcttttttgccacaaagaagattcttaccaagagtgggctttgtggaaacagctggtactgatgttcacctttatatatgtactagcattttccacgctgatgtttatgtactgtaaacagttctgcactcttgtacaaaagaaaaaaccacccggaattc

SEQ ID NO: 24 - protein, 465 aa, Homo sapiens
MFLATLYFALPLLDLLLSAEVSGGDRLDCVKASDQCLKEQSCSTKYRTLRQCVAGKETNFSLASGLEAKDECRSAMEALKQKSLYNCRCKRGMKKEKNCLRIYWSMYQSLQGNDLLEDSPYEPVNSRLSDIFRVVPFISDVFQQVEHIPKGNNCLDAAKACNLDDICKKYRSAYITPCTTSVSNDVCNRRKCHKALRQFFDKVPAKHSYGMLFCSCRDIACTERRRQTIVPVCSYEEREKPNCLNLQDSCKTNYICRSRLADFFTNCQPESRSVSSCLKENYADCLLAYSGLIGTVMTPNYIDSSSLSVAPWCDCSNSGNDLEECLKFLNFFKDNTCLKNAIQAFGNGSDVTVWQPAFPVQTTTATTTTALRVKNKPLGPAGSENEIPTHVLPPCANLQAQKLKSNVSGNTHLCISNGNYEKEGLGASSHITTKSMAAPPSCGLSPLLVLVVTALSTLLSLTETS

SEQ ID NO: 25 - DNA, 1576 bp, Homo sapiens
cttttgtttcgccatgcctagtctagtggtatctggaataatggaaagaaatgggggctttggagaactaggatgtttcgggggaagcgctaaggaccgagggctgctggaagacgagcgcgcccttcagctggctctcgatcaactctgcctcctgggtttgggggagccccccgcccccagggcgggcgaggacgggggaggtggggggggcggcgcccccgcgcagccgacagcccccccgcagccggcgccgccgccgccgcccgcggcgcccccggccgccccgacgacggcccccgcagcgcagacgccccagccccccaccgcccccaaaggggcgagcgacgccaagctctgcgctctctacaaagaggccgagctgcgcctgaagggcagcagcaacaccacggagtgtgttcccgtgcccacctccgagcacgtggccgagatcgtgggcaggcaaggctgcaagattaaggccttgagggccaagaccaacacctacatcaagacaccggtgaggggcgaggaaccagtgttcatggtgacagggcgacgggaggacgtggccacagcccggcgggaaatcatctcagcagcggagcacttctccatgatccgtgcctcccgcaacaagtcaggcgccgcctttggtgtggctcctgctctgcccggccaggtgaccatccgtgtgcgggtgccctaccgcgtggtggggctggtggtgggccccaaaggggcaaccatcaagcgcatccagcagcaaaccaacacatacattatcacaccaagccgtgaccgcgaccccgtgttcgagatcacgggtgccccaggcaacgtggagcgtgcgcgcgaggagatcgagacgcacatcgcggtgcgcactggcaagatcctcgagtacaacaatgaaaacgacttcctggcggggagccccgacgcagcaatcgatagccgctactccgacgcctggcgggtgcaccagcccggctgcaagcccctctccaccttccggcagaacagcctgggctgcatcggcgagtgcggagtggactctggctttgaggccccacgcctgggtgagcagggcggggactttggctacggcgggtacctctttccgggctatggcgtgggcaagcaggatgtgtactacggcgtggccgagactagccccccgctgtgggcgggccaggagaacgccacgcccacctccgtgctcttctcctctgcctcctcctcctcctcctcttccgccaaggcccgcgctgggcccccgggcgcacaccgctcccctgccacttccgcgggacccgagctggccggactcccgaggcgccccccgggagagccgctccagggcttctctaaacttggtgggggcggcctgcggagccccggcggcgggcgggattgcatggtctgctttgagagcgaagtgactgccgcccttgtgccctgcggacacaacctgttctgcatggagtgtgcagtacgcatctgcgagaggacggacccagagtgtcccgtctgccacatcacagccacgcaagccatccgaatattctcctaa

SEQ ID NO: 26 - protein, 524 aa, Homo sapiens
FCFAMPSLVVSGIMERNGGFGELGCFGGSAKDRGLLEDERALQLALDQLCLLGLGEPPAPRAGEDGGGGGGGAPAQPTAPPQPAPPPPPAAPPAAPTTAPAAQTPQPPTAPKGASDAKLCALYKEAELRLKGSSNTTECVPVPTSEHVAEIVGRQGCKIKALRAKTNTYIKTPVRGEEPVFMVTGRREDVATARREIISAAEHFSMIRASRNKSGAAFGVAPALPGQVTIRVRVPYRVVGLVVGPKGATIKRIQQQTNTYIITPSRDRDPVFEITGAPGNVERAREEIETHIAVRTGKILEYNNENDFLAGSPDAAIDSRYSDAWRVHQPGCKPLSTFRQNSLGCIGECGVDSGFEAPRLGEQGGDFGYGGYLFPGYGVGKQDVYYGVAETSPPLWAGQENATPTSVLFSSASSSSSSSAKARAGPPGAHRSPATSAGPELAGLPRRPPGEPLQGFSKLGGGGLRSPGGGRDCMVCFESEVTAALVPCGHNLFCMECAVRICERTDPECPVCHITATQAIRIFS

SEQ ID NO: 27 - DNA, 2070 bp, Homo sapiens
atgagcggtgcgggggtggcggctgggacgcggccccccagctcgccgaccccgggctctcggcgccggcgccagcgcccctctgtgggcgtccagtccttgaggccgcagagcccgcagctcaggcagagcgacccgcagaaacggaacctggacctggagaaaagcctgcagttcctgcagcagcagcactcggagatgctggccaagctccatgaggagatcgagcatctgaagcgggaaaacaagggtgagccggcgcggggccctaggccggccctgcctccccaggcacactcaacactgccgctcccgcagcacagaaacacagccatcaactccagcacacgcctgggctcagggggaacacaggacggggagcccctccagactgtccttgcccacctggctgcactggcccctgtatgccaacccagtgggtacaggttctgggggacctggacagatgccgctacctctagccgtggctggacgatgttatgcagccaagcacagcacgtgctgctctcgggaagcccagggcctgaggtcattgcagggcggcaggtggccacagggtgctccccagacctccctcctccaagtagagctgaaatgggaaggaacccctgggacagcccctgccctgctagatctttgcctcagattgctgctgtggccaggcccaggatttccagccctatggctctgagtcctcacatgctgggggcccaggggatatggacacactccatccagggatcccttcctgccatctgggcagcaaccatggggacaaagggaggaagcagagtcctgtttccttgccacttgtccaaggcacttccccatcctgacagcggcccccacccagcccaggatcctgggctgtggtctcaagctcacttcccattatctttggggctggggctgacatcaggaggacatctgactggtggatggagccagcctgggaacatcgcagctggggcagtgcctagggctctcccttcccagggagacatggagaagggggttgagggagggcccttccctagccgctgtggcaactccagtgagctgttctgggcaaagtgtggcccaagtcggcagccccagccctgcagtgctggggacgctgacaggacacgggaagaggccatgctttccctcgggacctgctgttccatgtgtcccaagccctcctgctttccagatggcccctcaggaaaccacctttccagggcctctgctcccttgggcgctcgctgggtctgcatcaacggagtgtgggtagagccgggaggacccagccctgccaggctgaaggagggctcctcacggacacacaggccaggaggcaagcgtgggcgtcttgcgggcggtagcgccgacactgtgcgctctcctgcagacagcctctccatgtcaagcttccagtctgtcaagtccatctctaattcagccaactctcaaggcaaggccaggccccagcccggctccttcaacaagcaagattcaaaagctgacgtctcccagaaggcggacctggaagaggagcccctacttcacaacagcaagctggacaaagttcctggggtacaagggcaggccagaaaggagaaagcagaggcctctaatgcaggagctgcctgtatggggaacagccagcaccagggcaggcagatgggggcgggggcacaccccccaatgatcctgccccttcccctgcgaaagcccaccacacttaggcagtgcgaagtgctcatccgcgagctgtggaataccaacctcctgcagacccaagagctgcggcacctcaagtccctcctggaagggagccagaggccccaggcagccccggaggaagctagctttcccagggaccaagaagccacgcatttccccaaggtctccaccaagagcctctccaagaaatgcctgagcccacctgtggcggagcgtgccatcctgcccgcactgaagcagaccccgaagaacaactttgccgagaggcagaagaggctgcaggcaatgcagaaacggcgcctgcatcgctcagtgctttga

SEQ ID NO: 28 - protein, 689 aa, Homo sapiens [sequence omitted]

SEQ ID NO: 29 - DNA, 3461 bp, Homo sapiens
ctcgtgccgaattcggcacgagaccgcgtgttcgcgcctggtagagatttctcgaagacaccagtgggcccgtgtggaaccaaacctgcgcgcgtggccgggccgtgggacaacgaggccgcggagacgaaggcgcaatggcgaggaagttatctgtaatcttgatcctgacctttgccctctctgtcacaaatccccttcatgaactaaaagcagctgctttcccccagaccactgagaaaattagtccgaattgggaatctggcattaatgttgacttggcaatttccacacggcaatatcatctacaacagcttttctaccgctatggagaaaataattctttgtcagttgaagggttcagaaaattacttcaaaatataggcatagataagattaaaagaatccatatacaccatgaccacgaccatcactcagaccacgagcatcactcagaccatgagcgtcactcagaccatgagcatcactcagaccacgagcatcactctgaccatgatcatcactctcaccataatcatgctgcttctggtaaaaataagcgaaaagctctttgcccagaccatgactcagatagttcaggtaaagatcctagaaacagccaggggaaaggagctcaccgaccagaacatgccagtggtagaaggaatgtcaaggacagtgttagtgctagtgaagtgacctcaactgtgtacaacactgtctctgaaggaactcactttctagagacaatagagactccaagacctggaaaactcttccccaaagatgtaagcagctccactccacccagtgtcacatcaaagagccgggtgagccggctggctggtaggaaaacaaatgaatctgtgagtgagccccgaaaaggctttatgtattccagaaacacaaatgaaaatcctcaggagtgtttcaatgcatcaaagctactgacatctcatggcatgggcatccaggttccgctgaatgcaacagagttcaactatctctgtccagccatcatcaaccaaattgatgctagatcttgtctgattcatacaagtgaaaagaaggctgaaatccctccaaagacctattcattacaaatagcctgggttggtggttttatagccatttccatcatcagtttcctgtctctgctgggggttatcttagtgcctctcatgaatcgggtgtttttcaaatttctcctgagtttccttgtggcactggccgttgggactttgagtggtgatgcttttttacaccttcttccacattctcatgcaagtcaccaccatagtcatagccatgaagaaccagcaatggaaatgaaaagaggaccacttttcagtcatctgtcttctcaaaacatagaagaaagtgcctattttgattccacgtggaagggtctaacagctctaggaggcctgtatttcatgtttcttgttgaacatgtcctcacattgatcaaacaatttaaagataagaagaaaaagaatcagaagaaacctgaaaatgatgatgatgtggagattaagaagcagttgtccaagtatgaatctcaactttcaacaaatgaggagaaagtagatacagatgatcgaactgaaggctatttacgagcagactcacaagagccctcccactttgattctcagcagcctgcagtcttggaagaagaagaggtcatgatagctcatgctcatccacaggaagtctacaatgaatatgtacccagagggtgcaagaataaatgccattcacatttccacgatacactcggccagtcagacgatctcattcaccaccatcatgactaccatcatattctccatcatcaccaccaccaaaaccaccatcctcacagtcacagccagcgctactctcgggaggagctgaaagatgccggcgtcgccactttggcctggatggtgataatgggtgatggcctgcacaatttcagcgatggcctagcaattggtgctgcttttactgaaggcttatcaagtggtttaagtacttctgttgctgtgttctgtcatgagttgcctcatgaattaggtgactttgctgttctactaaaggctggcatgaccgttaagcaggctgtcctttataatgcattgtcagccatgctggcgtatcttggaatggcaacaggaattttcattggtcattatgctgaaaatgtttctatgtggatatttgcacttactgctggcttattcatgtatgttgctctggttgatatggtacctgaaatgctgcacaatgatgctagtgaccatggatgtagccgctgggggtatttctttttacagaatgctgggatgcttttgggttttggaattatgttacttatttccatatttgaacataaaatcgtgtttcgtataaatttctagttaaggtttaaatgctagagtagcttaaaaagttgtcatagtttcagtaggtcatagggagatgagtttgtatgctgtactatgcagcgtttaaagttagtgggttttgtgatttttgtattgaatattgctgtctgttacaaagtcagttaaaggtacgttttaatatttaagttattctatcttggagataaaatctgtatgtgcaattcaccggtattaccagtttattatgtaaacaagagatttggcatgacatgttctgtatgtttcagggaaaaatgtctttaatgctttttcaagaactaacacagttattcctatactggattttaggtctctgaagaactgctggtgtttaggaataagaatgtgcatgaagcctaaaataccaagaaagcttatactgaatttaagcaaagaaataaaggagaaaagagaagaatctgagaattggggaggcatagattcttataaaaatcacaaaatttgttgtaaattagaggggagaaatttagaattaagtataaaaaggcagaattagtatagagtacattcattaaacatttttgtcaggattatttcccgtaaaaacgtagtgagcactctcatatactaattagtgtacatttaactttgtataatacagaaatctaaatatatttaatgaattcaagcaatatacacttgaccaagaaattggaatttcaaaatgttcgtgcgggttatataccagatgagtacagtgagtagtttatgtatcaccagactgggttattgccaagttatatatcaccaaaagctgtatgactggatgttctggttacctggtttacaaaattatcagagtagtaaaactttgatatatatgaggatattaaaactacactaagtatcatttgattcgattcagaaagtactttgatatctctcagtgcttcagtgctatcattgtgagcaattgtctttatatacggtactgtagccatactaggcctgtctgtggcattctctagatgtttcttttttacacaataaattccttatatcagcttg

SEQ ID NO: 30 - protein, 755 aa, Homo sapiens
MARKLSVILILTFALSVTNPLHELKAAAFPQTTEKISPNWESGINVDLAISTRQYHLQQLFYRYGENNSLSVEGFRKLLQNIGIDKIKRIHIHHDHDHHSDHEHHSDHERHSDHEHHSDHEHHSDHDHHSHHNHAASGKNKRKALCPDHDSDSSGKDPRNSQGKGAHRPEHASGRRNVKDSVSASEVTSTVYNTVSEGTHFLETIETPRPGKLFPKDVSSSTPPSVTSKSRVSRLAGRKTNESVSEPRKGFMYSRNTNENPQECFNASKLLTSHGMGIQVPLNATEFNYLCPAIINQIDARSCLIHTSEKKAEIPPKTYSLQIAWVGGFIAISIISFLSLLGVILVPLMNRVFFKFLLSFLVALAVGTLSGDAFLHLLPHSHASHHHSHSHEEPAMEMKRGPLFSHLSSQNIEESAYFDSTWKGLTALGGLYFMFLVEHVLTLIKQFKDKKKKNQKKPENDDDVEIKKQLSKYESQLSTNEEKVDTDDRTEGYLRADSQEPSHFDSQQPAVLEEEEVMIAHAHPQEVYNEYVPRGCKNKCHSHFHDTLGQSDDLIHHHHDYHHILHHHHHQNHHPHSHSQRYSREELKDAGVATLAWMVIMGDGLHNFSDGLAIGAAFTEGLSSGLSTSVAVFCHELPHELGDFAVLLKAGMTVKQAVLYNALSAMLAYLGMATGIFIGHYAENVSMWIFALTAGLFMYVALVDMVPEMLHNDASDHGCSRWGYFFLQNAGMLLGFGIMLLISIFEHKIVFRINF

SEQ ID NO: 31 - DNA, 3085 bp, Homo sapiens
gagcagccaaaaggcccgcggagtcgcgctgggccgccccggcgcagctgaaccgggggccgcgcctgccaggccgacgggtctggcccagcctggcgccaaggggttcgtgcgctgtggagacgcggagggtcgaggcggcgcggcctgagtgaaacccaatggaaaaagcatgacatttagaagtagaagacttagcttcaaatccctactccttcacttactaattttgtgatttggaaatatccgcgcaagatgttgacgttgcagacttgggtagtgcaagccttgtttattttcctcaccactgaatctacaggtgaacttctagatccatgtggttatatcagtcctgaatctccagttgtacaacttcattctaatttcactgcagtttgtgtgctaaaggaaaaatgtatggattattttcatgtaaatgctaattacattgtctggaaaacaaaccattttactattcctaaggagcaatatactatcataaacagaacagcatccagtgtcacctttacagatatagcttcattaaatattcagctcacttgcaacattcttacattcggacagcttgaacagaatgtttatggaatcacaataatttcaggcttgcctccagaaaaacctaaaaatttgagttgcattgtgaacgaggggaagaaaatgaggtgtgagtgggatggtggaagggaaacacacttggagacaaacttcactttaaaatctgaatgggcaacacacaagtttgctgattgcaaagcaaaacgtgacacccccacctcatgcactgttgattattctactgtgtattttgtcaacattgaagtctgggtagaagcagagaatgcccttgggaaggttacatcagatcatatcaattttgatcctgtatataaagtgaagcccaatccgccacataatttatcagtgatcaactcagaggaactgtctagtatcttaaaattgacatggaccaacccaagtattaagagtgttataatactaaaatataacattcaatataggaccaaagatgcctcaacttggagccagattcctcctgaagacacagcatccacccgatcttcattcactgtccaagaccttaaaccttttacagaatatgtgtttaggattcgctgtatgaaggaagatggtaagggatactggagtgactggagtgaagaagcaagtgggatcacctatgaagatagaccatctaaagcaccaagtttctggtataaaatagatccatcccatactcaaggctacagaactgtacaactcgtgtggaagacattgcctccttttgaagccaatggaaaaatcttggattatgaagtgactctcacaagatggaaatcacatttacaaaattacacagttaatgccacaaaactgacagtaaatctcacaaatgatcgctatctagcaaccctaacagtaagaaatcttgttggcaaatcagatgcagctgttttaactatccctgcctgtgactttcaagctactcaccctgtaatggatcttaaagcattccccaaagataacatgctttgggtggaatggactactccaagggaatctgtaaagaaatatatacttgagtggtgtgtgttatcagataaagcaccctgtatcacagactggcaacaagaagatggtaccgtgcatcgcacctatttaagagggaacttagcagagagcaaatgctatttgataacagttactccagtatatgctgatggaccaggaagccctgaatccataaaggcataccttaaacaagctccaccttccaaaggacctactgttcggacaaaaaaagtagggaaaaacgaagctgtcttagagtgggaccaacttcctgttgatgttcagaatggatttatcagaaattatactatattttatagaaccatcattggaaatgaaactgctgtgaatgtggattcttcccacacagaatatacattgtcctctttgactagtgacacattgtacatggtacgaatggcagcatacacagatgaaggtgggaaggatggtccagaattcacttttactaccccaaagtttgctcaaggagaaattgaagccatagtcgtgcctgtttgcttagcattcctattgacaactcttctgggagtgctgttctgctttaataagcgagacctaattaaaaaacacatctggcctaatgttccagatccttcaaagagtcatattgcccagtggtcacctcacactcctccaaggcacaattttaattcaaaagatcaaatgtattcagatggcaatttcactgatgtaagtgttgtggaaatagaagcaaatgacaaaaagccttttccagaagatctgaaatcattggacctgttcaaaaaggaaaaaattaatactgaaggacacagcagtggtattggggggtcttcatgcatgtcatcttctaggccaagcatttctagcagtgatgaaaatgaatcttcacaaaacacttcgagcactgtccagtattctaccgtggtacacagtggctacagacaccaagttccgtcagtccaagtcttctcaagatccgagtctacccagcccttgttagattcagaggagcggccagaagatctacaattagtagatcatgtagatggcggtgatggtattttgcccaggcaacagtacttcaaacagaactgcagtcagcatgaatccagtccagatatttcacattttgaaaggtcaaagcaagtttcatcagtcaatgaggaagattttgttagacttaaacagcagatttcagatcatatttcacaatcctgtggatctgggcaaatgaaaatgtttcaggaagtttctgcagcagatgcttttggtccaggtactgagggacaagtagaaagatttgaaacagttggcatggaggctgcgactgatgaaggcatgcctaaaagttacttaccacagactgtacggcaaggcggctacatgcctcagtgaaggactagtagttcctgctacaacttcagcagtacctataaagtaaagctaaaatgattttatctgtgaattc

SEQ ID NO: 32 - protein, 918 aa, Homo sapiens [sequence omitted]

SEQ ID NO: 33 - DNA, 2494 bp, Homo sapiens
agttggcgacatggtggcacccgtgctggagacttctcacgtgttttgctgcccaaaccgggtgcggggagtcctgaactggagctctgggcccagaggacttctggcctttggcacgtcctgctccgtggtgctctatgaccccctgaaaagggttgttgttaccaacttgaatggtcacaccgcccgagtcaattgcatacagtggatttgtaaacaggatggctccccttctactgaattagtttctggaggatctgataatcaagtgattcactgggaaatagaggataatcagcttttaaaagcagtgcatcttcaaggccatgaaggacctgtttatgcggtgcatgctgtttaccagaggaggacatcagatcctgcattatgtacactgatcgtttctgcagctgcagattctgctgttcgactctggtctaaaaagggtccagaagtaatgtgccttcagactttaaactttggaaatggatttgctttggctctctgcttatcttttttgccaaatactgatgtaccaatattagcatgtggcaatgatgattgcagaattcacatatttgctcaacaaaatgatcagtttcagaaagtgctttctctctgtggacatgaggattggattagaggagtggaatgggcagcctttggtagagatcttttcctagcaagctgttcacaagattgcctgataagaatatggaagctgtatataaagtcaacatctttagaaactcaggatgacgataacataagactgaaagaaaatacttttaccatagaaaatgaaagtgttaaaatagcatttgctgttactctggagacagtgctagccggtcatgaaaactgggtaaatgcagttcactggcaacctgtgttttacaaagatggtgtcctacagcagccagtgagattattatctgcttccatggataaaaccatgattctctgggctccagatgaagagtcaggagtttggctagaacaggttcgagtaggtgaagtaggtgggaatactttgggattttatgattgccagttcaatgaagatggctccatgatcattgctcatgctttccacggagcgttgcacctttggaaacagaatacagttaacccaagagagtggactccagagattgtcatttcaggacactttgatggtgtccaagacctagtctgggatccagaaggagaatttattatcactgttggtactgatcagacaactagactttttgctccatggaagagaaaagaccaatcacaggtgacttggcatgaaattgcaaggcctcagatacatgggtatgacctgaaatgtttggcaatgattaatcggtttcagtttgtatctggagcagatgaaaaagttcttcgggttttttctgcacctcggaattttgtggaaaatttttgtgccattacaggacaatcactgaatcatgtgctctgtaatcaagatagtgatcttccagaaggagccactgtccctgcattgggattatcaaataaagctgtctttcagggagatatagcttctcagccttctgatgaagaggagctgttaactagtactggttttgagtatcagcaggtggcctttcagccctccatacttactgagcctcccactgaggatcatcttctgcagaatactttgtggcctgaagttcaaaaactatatgggcacggttatgaaatattttgtgttacttgtaacagttcaaagactctgcttgcctcagcttgtaaggcagctaagaaagagcatgcagctatcattctttggaacactacatcttggaaacaggtgcagaatttagttttccacagtttgacagtcacgcagatggccttctcacctaatgagaagttcttactagctgtttccagagatcgaacctggtcattgtggaaaaagcaggatacaatctcacctgagttcgagccagtttttagtctttttgccttcaccaacaaaattacttctgtgcacagtagaattatttggtcttgtgattggagtcctgacagcaagtatttcttcactgggagtcgagacaaaaaggtggttgtctggggtgagtgcgactccactgatgactgtattgagcacaacattggcccctgctcctcagtcctggacgtgggtggggctgtgacagctgtcagcgtctgcccagtgctccacccttctcaacgatacgtggttgcagtaggattggagtgtggaaagatttgcttatatacctggaaaaagactgatcaagttccagaaataaatgactggacccactgtgtagaaacaagtcaaagccaaagtcatacactggctatcagaaaattatgctggaagaattgcagtggaaaaactgaacagaaggaagcagaaggtgctgagtggttacactttgcaagctgtggtgaagatcacactgtgaagatacacagagtcaataaatgtgcactgtaatgg

SEQ ID NO: 34 - protein, 826 aa, Homo sapiens
MVAPVLETSHVFCCPNRVRGVLNWSSGPRGLLAFGTSCSVVLYDPLKRVVVTNLNGHTARVNCIQWICKQDGSPSTELVSGGSDNQVIHWEIEDNQLLKAVHLQGHEGPVYAVHAVYQRRTSDPALCTLIVSAAADSAVRLWSKKGPEVMCLQTLNFGNGFALALCLSFLPNTDVPILACGNDDCRIHIFAQQNDQFQKVLSLCGHEDWIRGVEWAAFGRDLFLASCSQDCLIRIWKLYIKSTSLETQDDDNIRLKENTFTIENESVKIAFAVTLETVLAGHENWVNAVHWQPVFYKDGVLQQPVRLLSASMDKTMILWAPDEESGVWLEQVRVGEVGGNTLGFYDCQFNEDGSMIIAHAFHGALHLWKQNTVNPREWTPEIVISGHFDGVQDLVWDPEGEFIITVGTDQTTRLFAPWKRKDQSQVTWHEIARPQIHGYDLKCLAMINRFQFVSGADEKVLRVFSAPRNFVENFCAITGQSLNHVLCNQDSDLPEGATVPALGLSNKAVFQGDIASQPSDEEELLTSTGFEYQQVAFQPSILTEPPTEDHLLQNTLWPEVQKLYGHGYEIFCVTCNSSKTLLASACKAAKKEHAAIILWNTTSWKQVQNLVFHSLTVTQMAFSPNEKFLLAVSRDRTWSLWKKQDTISPEFEPVFSLFAFTNKITSVHSRIIWSCDWSPDSKYFFTGSRDKKVVVWGECDSTDDCIEHNIGPCSSVLDVGGAVTAVSVCPVLHPSQRYVVAVGLECGKICLYTWKKTDQVPEINDWTHCVETSQSQSHTLAIRKLCWKNCSGKTEQKEAEGAEWLHFASCGEDHTVKIHRVNKCAL

SEQ ID NO: 35 - DNA, 4694 bp, Homo sapiens
tgctgcgaggatgctgcctgggcggctgtgctgggtgccgctcctgctggcgctgggcgtggggagcggcagcggcggtggcggggacagccggcagcgccgcctcctcgcggctaaagtcaataagcacaagccatggatcgagacttcatatcatggagtcataactgagaacaatgacacagtcattttggacccaccactggtagccctggataaagatgcaccggttccttttgcaggggaaatctgtgcgttcaagatccatggccaggagctgccctttgaggctgtggtgctcaacaagacatcaggagagggccggctccgtgccaagagccccattgactgtgagttgcagaaggagtacacattcatcatccaggcctatgactgtggtgctgggccccacgagacagcctggaaaaagtcacacaaggccgtggtccatatacaggtgaaggatgtcaacgagtttgctcccaccttcaaagagccagcctacaaggctgttgtgacggagggcaagatctatgacagcattctgcaggtggaggccattgacgaggactgctccccacagtacagccagatctgcaactatgaaatcgtcaccacagatgtgccttttgccatcgacagaaatggcaacatcaggaacactgagaagctgagctatgacaaacaacaccagtatgagatcctggtgaccgcctacgactgtggacagaagcccgctgctcaggacaccctggtgcaggtggatgtgaagccagtttgcaagcctggctggcaagactggaccaagaggattgagtaccagcctggctccgggagcatgcccctgttccccagcatccacctggagacgtgcgatggagccgtgtcttccctccagatcgtcacagagctgcagactaattacattgggaagggttgtgaccgggagacctactctgagaaatcccttcagaagttatgtggagcctcctctggcatcattgacctcttgccatcccctagcgctgccaccaactggactgcaggactgctggtggacagcagtgagatgatcttcaagtttgacggcaggcagggtgccaaaatccccgatgggattgtgcccaagaacctgaccgatcagttcaccatcaccatgtggatgaaacacggccccagccctggtgtgagagccgagaaggaaaccatcctctgcaactcagacaaaaccgaaatgaaccggcatcactatgccctgtatgtgcacaactgccgcctcgtctttctcttgcggaaggacttcgaccaggctgacacctttcgccccgcggagttccactggaagctggatcagatttgtgacaaagagtggcactactatgtcatcaatgtggagtttcctgtggtaaccttatacatggatggagcaacatatgaaccatacctggtgaccaacgactggcccattcatccatctcacatagccatgcaactcacagtcggcgcttgttggcaaggaggagaagtcaccaaaccacagtttgctcagttctttcatggaagcctggccagtctcaccatccgccctggcaaaatggaaagccagaaggtgatctcctgcctgcaggcctgcaaggaagggctggacattaattccttggaaagccttggccaaggaataaagtatcacttcaacccctcgcagtccatcctggtgatggaaggtgacgacattgggaacattaaccgtgctctccagaaagtctcctacatcaactccaggcagttcccaacggcgggtgtgcggcgcctcaaagtatcctccaaagtccagtgctttggggaagacgtatgcatcagtatccctgaggtagatgcctatgtgatggtcctccaggccatcgagccccggatcaccctccggggcacagaccacttctggagacctgctgcccagtttgaaagtgccaggggagtgaccctcttccctgatatcaagattgtgagcaccttcgccaaaaccgaagcccccggggacgtgaaaaccacagaccccaaatcagaagtcttagaggaaatgcttcataacttagatttctgtgacattttggtgatcggaggggacttggacccaaggcaggagtgcttggagctcaaccacagtgagctccaccaacgacacctggatgccactaattctactgcaggctactccatctacggtgtgggctccatgagccgctatgagcaggtgctacatcacatccgctaccgcaactggcgtccggcttcccttgaggcccggcgtttccggattaagtgctcagaactcaatgggcgctacactagcaatgagttcaacttggaggtcagcatccttcatgaagaccaagtctcagataaggagcatgtcaatcatctgattgtgcagcctcccttcctccagtctgtccatcatcctgagtcccggagtagcatccagcacagttcagtggtcccaagcattgccacagtggtcatcatcatctccgtgtgcatgcttgtgtttgtcgtggccatgggtgtgtaccgggtccggatcgcccaccagcacttcatccaggagactgaggctgccaaggaatctgagatggactgggacgattctgcgctgactatcacagtcaaccccatggagaaacatgaaggaccagggcatggggaagatgagactgagggagaagaggaggaagaagccgaggaagaaatgagctccagcagtggctctgacgacagcgaagaggaggaggaggaggaagggatgggcagaggcagacatgggcagaatggagccaggcaagcccagctggagtgggatgactccaccctcccctactagtgcccaggggtctgctgcctggcccacatgtcccttttgtaaaccctgacccagtgtatgcccatgtctatcatacctcacctctgatgtctgtgacatgtctgggaaggccttctccagcttcctggagcccaccctttaagccttgggcactccctgtgtttcatccatggggaagttccaagaagcccagcatggccatcagtgaggacttcagggtagactttgtcctgtagcctccacttctgccctaagttccccagcatcctgactacctgtctgcagagtttgcctttgttttttcctgcagggaagaaggcccacctttgtgtcactcacctccccaggctcagagtccccaaggccctggggttccaactcactgtgcgtctcctccacacagaccagtaggttctcctatgctgactccaggttgcttcatacaaggagggtggttgaacttcacacacgtaaggtcttagtgcttaacagtttaaaggaaagtccttgttgaggcagaactaagtttacagggaaaggtacacacattctctctctctctctctctctgtctatctagttccccagcttggagagcctttccccttgcttctttctgaggccatataagcttataagaaaagtcccaaaccaagaataggtccttggccacaagcagggtctgatcccccatcagagctatctgagcctgcctgtctgggcacctgctgcaaccatgcagctaccctgccaggggcactcagcaaacagaaccacagggcccaggaggcattccacacaggcactgccccaggacaacacaacaaggacagtcacaacaaggacaacaaggacacaacacaacacacaacaaggacagtcacaacaagcctagagccagaaagcagatggaaatgctaatgaggtcaaacgtaggcttcatggtgggtggagtgggggtggctgggctcccccaggacagaggggaccctgaggttggcaaggctctcaccactcagccttatggtcccttatctcctatcttccctcttgagaaaatacacgctttctgcatgtattagaaacgcacgagctccaccaagtctacaatgaaagtttgaaatttaactgcaaggaattagaagcatatttgcaatcattgcagcttcttctttcttctgctcataaaaggaggaacactttagatagagggcaaatatatctgaaaacctaatttctttctttttttgataaggaaatcttttccatctccatcctaacatgcacaacctgtgaagagaattgtttctatagtaactggtctgtgatcttttgtggccaagagaatagcaggcaagaattagggccttgacagaatttccacgaagctctgagaacatgtttgtttcgaatgtctgattcctctttgtcatcaatgtgtatgctctgtccccatccttcactcctcctcaagctcacaccaattggtttggcacaggcacagagctggtccctagttaagtggcatttatgttaaaaaaaaatagttcagaatctcagccttttctttgtgtcatcaaaacagcttaagaaggggactactgccaatgtcctctagtctgacctccacccagggaggacccatggcaggtcttttcaactttctgattcatgagaacaaccttgtgaagcttttcccacctcctaaagtgttttctgcatctgttccttcctttggacctcacaacaaatcctgtgaagtaactgagacatctgttgttagatacatttttgtgatgagtaaactgaggcttcg

SEQ ID NO: 36 - protein, 955 aa, Homo sapiens
MLPGRLCWVPLLLALGVGSGSGGGGDSRQRRLLAAKVNKHKPWIETSYHGVITENNDTVILDPPLVALDKDAPVPFAGEICAFKIHGQELPFEAVVLNKTSGEGRLRAKSPIDCELQKEYTFIIQAYDCGAGPHETAWKKSHKAVVHIQVKDVNEFAPTFKEPAYKAVVTEGKIYDSILQVEAIDEDCSPQYSQICNYEIVTTDVPFAIDRNGNIRNTEKLSYDKQHQYEILVTAYDCGQKPAAQDTLVQVDVKPVCKPGWQDWTKRIEYQPGSGSMPLFPSIHLETCDGAVSSLQIVTELQTNYIGKGCDRETYSEKSLQKLCGASSGIIDLLPSPSAATNWTAGLLVDSSEMIFKFDGRQGAKIPDGIVPKNLTDQFTITMWMKHGPSPGVRAEKETILCNSDKTEMNRHHYALYVHNCRLVFLLRKDFDQADTFRPAEFHWKLDQICDKEWHYYVINVEFPVVTLYMDGATYEPYLVTNDWPIHPSHIAMQLTVGACWQGGEVTKPQFAQFFHGSLASLTIRPGKMESQKVISCLQACKEGLDINSLESLGQGIKYHFNPSQSILVMEGDDIGNINRALQKVSYINSRQFPTAGVRRLKVSSKVQCFGEDVCISIPEVDAYVMVLQAIEPRITLRGTDHFWRPAAQFESARGVTLFPDIKIVSTFAKTEAPGDVKTTDPKSEVLEEMLHNLDFCDILVIGGDLDPRQECLELNHSELHQRHLDATNSTAGYSIYGVGSMSRYEQVLHHIRYRNWRPASLEARRFRIKCSELNGRYTSNEFNLEVSILHEDQVSDKEHVNHLIVQPPFLQSVHHPESRSSIQHSSVVPSIATVVIIISVCMLVFVVAMGVYRVRIAHQHFIQETEAAKESEMDWDDSALTITVNPMEKHEGPGHGEDETEGEEEEEAEEEMSSSSGSDDSEEEEEEEGMGRGRHGQNGARQAQLEWDDSTLPY

SEQ ID NO: 37 - DNA, 1284 bp, Homo sapiens
gggagggagagaggcgcgcgggtgaaaggcgcattgatgcagcctgcggcggcctcggagcgcggcggagccagacgctgaccacgttcctctcctcggtctcctccgcctccagctccgcgctgcccggcagccgggagccatgcgaccccagggccccgccgcctccccgcagcggctccgcggcctcctgctgctcctgctgctgcagctgcccgcgccgtcgagcgcctctgagatccccaaggggaagcaaaaggcgcagctccggcagagggaggtggtggacctgtataatggaatgtgcttacaagggccagcaggagtgcctggtcgagacgggagccctggggccaatggcattccgggtacacctgggatcccaggtcgggatggattcaaaggagaaaagggggaatgtctgagggaaagctttgaggagtcctggacacccaactacaagcagtgttcatggagttcattgaattatggcatagatcttgggaaaattgcggagtgtacatttacaaagatgcgttcaaatagtgctctaagagttttgttcagtggctcacttcggctaaaatgcagaaatgcatgctgtcagcgttggtatttcacattcaatggagctgaatgttcaggacctcttcccattgaagctataatttatttggaccaaggaagccctgaaatgaattcaacaattaatattcatcgcacttcttctgtggaaggactttgtgaaggaattggtgctggattagtggatgttgctatctgggttggcacttgttcagattacccaaaaggagatgcttctactggatggaattcagtttctcgcatcattattgaagaactaccaaaataaatgctttaattttcatttgctacctctttttttattatgccttggaatggttcacttaaatgacattttaaataagtttatgtatacatctgaatgaaaagcaaagctaaatatgtttacagaccaaagtgtgatttcacactgtttttaaatctagcattattcattttgcttcaatcaaaagtggtttcaatattttttttagttggttagaatactttcttcatagtcacattctctcaacctataatttggaatattgttgtggtcttttgttttttctcttagtatagcatttttaaaaaaatataaaagctaccaatctttgtacaatttgtaaatgttaagaattttttttatatctgttaaataaaaattatttccaacaaccttaaaaaaaaaaaaaaaaa

SEQ ID NO: 38 - protein, 243 aa, Homo sapiens
MRPQGPAASPQRLRGLLLLLLLQLPAPSSASEIPKGKQKAQLRQREVVDLYNGMCLQGPAGVPGRDGSPGANGIPGTPGIPGRDGFKGEKGECLRESFEESWTPNYKQCSWSSLNYGIDLGKIAECTFTKMRSNSALRVLFSGSLRLKCRNACCQRWYFTFNGAECSGPLPIEAIIYLDQGSPEMNSTINIHRTSSVEGLCEGIGAGLVDVAIWVGTCSDYPKGDASTGWNSVSRIIIEELPK

SEQ ID NO: 39 - DNA, 2723 bp, Homo sapiens
ggaggctgaaatccccagacgccggttttctgggctgggctttctgcttactcactccttctccctctttctggattttaccgaccgttcgcgaaacagctttccacacaatggagcttcatgtcctcgtgcaggaagtactcatcgactgatgtggcagactttgctccctgacaaaactaaagaactctcctattcatggaggcgaacactgaggatgctttccacatgaaccctgaagtgaacttctgatacatttcctgcagcaagagaaggcagccaacatgaaggaaaatgtggcatctgcaaccgttttcactctgctactttttctcaacacctgccttctgaatggacagttacctcctggaaaacctgagatctttaaatgtcgttctcccaataaggaaacattcacctgctggtggaggcctgggacagatggaggacttcctaccaattattcactgacttaccacagggaaggagagacactcatgcatgaatgtccagactacataaccggtggccccaactcctgccactttggcaagcagtacacctccatgtggaggacatacatcatgatggtcaatgccactaaccagatgggaagcagtttctcggatgaactttatgtggacgtgacttacatagttcagccagaccctcctttggagctggctgtggaagtaaaacagccagaagacagaaaaccctacctgtggattaaatggtctccacctaccctgattgacttaaaaactggttggttcacgctcctgtatgaaattcgattaaaacccgagaaagcagctgagtgggagatccattttgctgggcagcaaacagagtttaagattctcagcctacatccaggacagaaataccttgtccaggttcgctgcaaaccagaccatggatactggagtgcatggagtccagcgaccttcattcagatacctagtgacttcaccatgaatgatacaaccgtgtggatctctgtggctgtcctttctgctgtcatctgtttgattattgtctgggcagtggctttgaagggctatagcatggtgacctgcatctttccgccagttcctgggccaaaaataaaaggatttgatgctcatctgttggagaagggcaagtctgaagaactactgagtgccttgggatgccaagactttcctcccacttctgactatgaggacttgctggtggagtatttagaagtagatgatagtgaggaccagcatctaatgtcagtccattcaaaagaacacccaagtcaaggtatgaaacccacatacctggatcctgacactgactcaggccgggggagctgtgacagcccttcccttttgtctgaaaagtgtgaggaaccccaggccaatccctccacattctatgatcctgaggtcattgagaagccagagaatcctgaaacaacccacacctgggacccccagtgcataagcatggaaggcaaaatcccctattttcatgctggtggatccaaatgttcaacatggcccttaccacagcccagccagcacaaccccagatcctcttaccacaatattactgatgtgtgtgagctggctgtgggccctgcaggtgcaccggccactctgttgaatgaagcaggtaaagatgctttaaaatcctctcaaaccattaagtctagagaagagggaaaggcaacccagcagagggaggtagaaagcttccattctgagactgaccaggatacgccctggctgctgccccaggagaaaaccccctttggctccgctaaacccttggattatgtggagattcacaaggtcaacaaagatggtgcattatcattgctaccaaaacagagagagaacagcggcaagcccaagaagcccgggactcctgagaacaataaggagtatgccaaggtgtccggggtcatggataacaacatcctggtgttggtgccagatccacatgctaaaaacgtggcttgctttgaagaatcagccaaagaggccccaccatcacttgaacagaatcaagctgagaaagccctggccaacttcactgcaacatcaagcaagtgcaggctccagctgggtggtttggattacctggatcccgcatgttttacacactcctttcactgatagcttgactaatggaatgattggttaaaatgtgatttttcttcaggtaacactacagagtacgtgaaatgctcaagaatgtagtcagactgacactactaaagctcccagctcctttcatgctccatttttaaccacttgcctctttctccagcagctgattccagaacaaatcattatgtttcctaactgtgatttgtagatttactttttgctgttagttataaaactatgtgttcaatgaaataaaagcacactgcttagtattcttgagggacaatgccaataggtatatcctctggaaaaggctttcatgatttggcatgggacagacggaaatgaaattgtcaaaattgtttaccatagaaagatgacaaaagaaaattttccacataggaaaatgccatgaaaattgcttttgaaaaacaactgcataacctttacactcctcgtccattttattaggattacccaaatataaccatttaaagaaagaatgcattccagaacaaattgtttacataagttcctataccttactgacacattgctgatatgcaagtaagaaat

SEQ ID NO: 40 - protein, 622 aa, Homo sapiens
MKENVASATVFTLLLFLNTCLLNGQLPPGKPEIFKCRSPNKETFTCWWRPGTDGGLPTNYSLTYHREGETLMHECPDYITGGPNSCHFGKQYTSMWRTYIMMVNATNQMGSSFSDELYVDVTYIVQPDPPLELAVEVKQPEDRKPYLWIKWSPPTLIDLKTGWFTLLYEIRLKPEKAAEWEIHFAGQQTEFKILSLHPGQKYLVQVRCKPDHGYWSAWSPATFIQIPSDFTMNDTTVWISVAVLSAVICLIIVWAVALKGYSMVTCIFPPVPGPKIKGFDAHLLEKGKSEELLSALGCQDFPPTSDYEDLLVEYLEVDDSEDQHLMSVHSKEHPSQGMKPTYLDPDTDSGRGSCDSPSLLSEKCEEPQANPSTFYDPEVIEKPENPETTHTWDPQCISMEGKIPYFHAGGSKCSTWPLPQPSQHNPRSSYHNITDVCELAVGPAGAPATLLNEAGKDALKSSQTIKSREEGKATQQREVESFHSETDQDTPWLLPQEKTPFGSAKPLDYVEIHKVNKDGALSLLPKQRENSGKPKKPGTPENNKEYAKVSGVMDNNILVLVPDPHAKNVACFEESAKEAPPSLEQNQAEKALANFTATSSKCRLQLGGLDYLDPACFTHSFH

SEQ ID NO: 41 - DNA, 1572 bp, Homo sapiens
atgacccaaaataaattaaagctttgttccaaagccaatgtgtatactgaagtgcctgatggaggatggggctgggcggtagctgtttcatttttcttcgttgaagtcttcacctacggcatcatcaagacatttggtgtcttctttaatgacttaatggacagttttaatgaatccaatagcaggatctcatggataatctcaatctgtgtgtttgtcttaacattttcagctcccctcgccacagtcctgagcaatcgtttcggacaccgtctggtagtgatgttgggggggctacttgtcagcaccgggatggtggccgcctccttctcacaagaggtttctcatatgtacgtcgccatcggcatcatctctggtctgggatactgctttagttttctcccaactgtaaccatcctatcacaatattttggcaaaagacgttccatagtcactgcagttgcttccacaggagaatgtttcgctgtgtttgctttcgcaccagcaatcatggctctgaaggagcgcattggctggagatacagcctcctcttcgtgggcctactacagttaaacattgtcatcttcggagcactgctcagacccatctttatcagaggaccagcgtcaccgaaaatagtcatccaggaaaatcggaaagaagcgcagtatatgcttgaaaatgagaaaacacgaacctcaatagactccattgactcaggagtagaactaactacctcacctaaaaatgtgcctactcacactaacctggaactggagccgaaggccgacatgcagcaggtcctggtgaagaccagccccaggccaagcgaaaagaaagccccgctattagacttctccattttgaaagagaaaagttttatttgttatgcattatttggtctctttgcaacactgggattctttgcaccttccttgtacatcattcctctgggcattagtctgggcattgaccaggaccgcgctgcttttttattatctacgatggccattgcagaagttttcggaaggatcggagctggttttgtcctcaacagggagcccattcgtaagatttacattgagctcatctgcgtcatcttattgactgtgtctctgtttgcctttacttttgctactgaattctggggtctaatgtcatgcagcatattttttgggtttatggttggaacaataggagggactcacattccactgcttgctgaggatgatgtcgtgggcattgagaagatgtcttctgcagctggggtctacatcttcattcagagcatagcaggactggctggaccgccccttgcaggtttgttggtggaccaaagtaagatctacagcagggccttctactcctgcgcagctggcatggccctggctgctgtgtgcctcgccctggtgagaccgtgtaagatgggactgtgccagcatcatcactcaggtgaaacaaaggtagtgagccatcgtgggaagactttacaggacatacctgaagactttctggaaatggatcttgcaaaaaatgagcacagagttcacgtgcaaatggagccggtatga

SEQ ID NO: 42 - protein, 523 aa, Homo sapiens
MTQNKLKLCSKANVYTEVPDGGWGWAVAVSFFFVEVFTYGIIKTFGVFFNDLMDSFNESNSRISWIISICVFVLTFSAPLATVLSNRFGHRLVVMLGGLLVSTGMVAASFSQEVSHMYVAIGIISGLGYCFSFLPTVTILSQYFGKRRSIVTAVASTGECFAVFAFAPAIMALKERIGWRYSLLFVGLLQLNIVIFGALLRPIFIRGPASPKIVIQENRKEAQYMLENEKTRTSIDSIDSGVELTTSPKNVPTHTNLELEPKADMQQVLVKTSPRPSEKKAPLLDFSILKEKSFICYALFGLFATLGFFAPSLYIIPLGISLGIDQDRAAFLLSTMAIAEVFGRIGAGFVLNREPIRKIYIELICVILLTVSLFAFTFATEFWGLMSCSIFFGFMVGTIGGTHIPLLAEDDVVGIEKMSSAAGVYIFIQSIAGLAGPPLAGLLVDQSKIYSRAFYSCAAGMALAAVCLALVRPCKMGLCQHHHSGETKVVSHRGKTLQDIPEDFLEMDLAKNEHRVHVQMEPV

SEQ ID NO: 43 - DNA, 3690 bp, Homo sapiens
atgctgtctggcttcttgatgagtcccagtacccagcacagagcacagtacactcccggaggaaagaaacttccgtgggaggcttccatcggtgcgcacacctcccgagggcgaggcagcgaccgggagagggagagccggccggaggctgccgggctcctgtgggaccgcgctgcagccggggaggcggagaaggggaaccggggcgagccgcccgcctggatccgcgcccagcagcagccgcggccgccgccagctgggcaggctcccgggactgcggctgggggcgcgcaggaccctcgcctgcgtcctggacgttcccgggggagggtccggttgccagtgaaacctccagaggcttccggacgacagccccgggggccttctgactgcatcccgagatttccatcagcgagtgcaactcataaggcagtccctaaggggaccgggccaccggctgaggacggggatggcttaggagctcctggacctagggcccggcgtcgtcgcctcctgggcgtcgcggcagaggggagtggcccgcgcggaaagcgccgcgggacagtcagtgacgaggcccgggggtcgccggggccacgacttctcggagaccgtcctgcgctctctggagacgcgctgtccgcgcccagggtggtgccatgtggggcgctcgccgctcgtccgtctcctcatcctggaacgccgcttcgctcctgcagctgctgctggctgcgctgctggcggcgggggcgagggcccagcggcgagtactgccacggctggctggacgcgcagggcgtctggcgcatcggcttccagtgtcccgagcgcttcgacggcggcgacgccaccatctgctgcggcagctgcgcgttgcgctactgctgctccagcgccgaggcgcgcctggaccagggcggctgcgacaatgaccgccagcagggcgctggcgagcctggccgggcggacaaagacgggccccgacggctcggcagggcttcatgtcttaggggtacccaaggagacggcgagggtgcgcccccacccgtgagggcctggcagcggtgctcccctgaaggctccccgaaaggaaggcagctcctcagggctttcccggggctgctgccccgtgccagacgccgcggattcccatcttctccacgcggcggcccctctcccctgcagcggcccgccttgcccatctacgtgccgttcctcattgttggctccgtgtttgtcgcctttatcatcttggggtccctggtggcagcctgttgctgcagatgtctccggcctaagcaggatccccagcagagccgagccccagggggtaaccgcttgatggagaccatccccatgatccccagtgccagcacctcccgggggtcgtcctcacgccagtccagcacagctgccagttccagctccagcgccaactccggggcccgggcgcccccaacaaggtcacagaccaactgttgcttgccggaagggaccatgaacaacgtgtatgtcaacatgcccacgaatttctctgtgctgaactgtcagcaggccacccagattgtgccacatcaagggcagtatctgcatcccccatacgtggggtacacggtgcagcacgactctgtgcccatgacagctgtgccacctttcatggacggcctgcagcctggctacaggcagattcagtcccccttccctcacaccaacagtgaacagaagatgtacccagcggtgactgtataaccgagagtcactggtgggttcctttactgaagggagacgaaggcaggggtggattctcgaggtggaagtccgcacatgtcggtggtatttatggcacgattcctttggatggcttcatttgcccccagactgtatgaaaacatctccgaattagcatttctggatatgtttcatccagggtatcattgatttatgatggaaaaccggcctcagctggagatgactgtgatgttgctgatgggtgtataacaaatgcttgagtccgaagtgcccttgagatatggttgacgaaagaattttataaactgataaattaaggatttttattatgttgttattattatttcttttttgttgttgactgcacaggatcaaaatgcctgttatctcccttttactgggactttttttttttttttttttttttttaatcagacagggtcttgctctgttgcccaggctggagtgcagtggtgcgatctcggctcactgcaacttcagcctcctggattcaggcaacactcctgcctcagcctcccacgtggctgggattacaggtgcctgcccccatggctaattttttgtattttttgtagagatggggtttcaccatgttggctgggctggtctcactctcctgacctcaagcaatctgcctgtctcagcctcccaaagtgctgggattacaggcgtgagccaccgcccccagcctgagcctttttttttttctaatgcatccaaggttaaggggaagacgcaaataacaggactattctaaaaggaaacctgtttgaactctgtgagatcagtcatcagtctcagtattccacaggcacaccttaatttcattgtaaaaagatatatatattttgtctatttttgtgcttttgggggcctattttgtgcttttttaccttatgtagagatcttattacaaagtgattttctacattaaaaagagactgaaataaattgtatagttacttaactaatgaagacatttcagaactctgggatgattttaatcttgaagtagtaggtggtatagtcataaaaccattcatccccttcttgattgtatcttaattttctggctttaaggtgacatctgagaggtaatgcattcttttttatattgaaatcataaactatcacccgctgcttctctgagttacttttaattttgccttgtggttatggtttggcgtttccttctgtttggttttcagagccccatgtctatatagtcctgagtgcaagtaattactatacttgtaaatgaagatcagtatttctgcctagatctgataaaaaaattttcttgtcttagttataaaaattcaaagaaatgtgttacaaagatacttagtatagctcctcagccataacctgagacttgggatgaaatttaaaccagatacgatttactttgcagatcataaggctttttatactcttgttatcaaaatggcttatttttcaggcactaaggattgttaagagaaaagcttttcaacgaaggattgcctttcttctcccacactgttcttgatttcctctctctttcaggcctcaacaggcactgtattcattgccaatgttccaaattatcaaattcaagtgaatttatttgtgtgttctttacttatataaaaaaagataactttaaggatgtgcaagtacatttccaactgctagcacaaccagtattttgtaattaaacaaatcgctgtatggtatggtcttctacacatttatgtctatagatatctatcgatcatctttctattctgtttcatgactgaataatgtaaaaccagtgttggcaattggtatcatcaatgatactcattttttaataaccaaaggcaggggaaaatcattttacttattaataaatattttatgatgtgaaaaaaaaaaaaaaaaaaaaaaaaaaaa

SEQ ID NO: 44 - protein, 582 aa, Homo sapiens [sequence omitted]

SEQ ID NO: 45 - DNA, 3857 bp, Homo sapiens
ggccgagcctcggcggcggcggtagcggcggcggcgacgctgacacctcccaccatggacagcttcgacttagccctgctccaggaatgggacctcgagtcactgtgtgtctatgaaccagatagaaatgcattacggaggaaagaacgagaaagaagaaatcaagaaactcaacaggatgatggcacgtttaattctagttactctctcttcagtgagccctacaagactaacaagggggatgaactctccaaccggatccagaacactttaggcaattatgatgaaatgaaagactttttaactgatagaaccaatcagagtcatctcgttggagttcccaaacctggggttcctcagactcctgtgaacaagatcgatgaacattttgttgcagattcaagagcccagaaccagccctcgtctatctgtagcactacaacttccacaccagcagctgtccccgtgcagcagagtaaaagaggcactatgggctggcagaaggctgggcacccaccctctgacggccaacagagagcaacacaacagggctctctcaggaccttgcttggagatggtgttggcagacagcagcctcgggccaaacaagtgtgcaatgtggaggtgggccttcagacccaggagaggccacctgccatggcggccaagcacagcagcagcggacactgtgttcagaactttcctccatccctagcttcaaaacccagcctggtccagcagaaaccgaccgcgtatgtgaggccaatggacggccaagatcaggcccctgatgagtctcctaagctgaagtcgtcttcggaaaccagcgtgcactgcacatcatacaggggagtccctgccagcaagccggagcctgccagagccaaggccaagctctccaagttcagcatccccaagcagggggaggagagtagatctggagaaaccaacagctgtgttgaagaaataatccgggagatgacctggcttccaccactttctgctattcaagcacctggcaaagtggaaccaaccaaatttccatttccaaataaggactctcagcttgtatcctctggacacaataatccaaagaaaggtgatgcagagccagagagtccagacaatggcacatcgaatacatcaatgctggaagatgaccttaagctaagcagtgatgaagaggagaatgaacagcaggcagctcagagaacggctctccgcgctctctctgacagcgccgtggtccagcagcccaactgcagaacctcggtgccttccagcaagggcagcagcagcagcagcagcagcggcacgagcagctcctccagcgactcagagagcagctccggatctgactcggagaccgagagcagctccagcgagagtgagggcagcaagcccccccacttctccagccccgaggctgaaccggcatcctctaacaagtggcagctggataaatggctaaacaaagttaatccccacaagcctcctattctgatccaaaatgaaagccacgggtcagagagcaatcagtactacaacccggtgaaagaggacgtccaggactgtgggaaagtccccgacgtttgccagcccagcctgagagagaaggagatcaagagcacttgcaaggaggagcaaaggccaaggacagccaacaaggcccctgggagtaaaggcgtgaagcagaagtccccgcccgcggccgtggccgtggcggtgagcgcagccgccccgccacccgcagtgccctgtgcgcccgcggagaacgcgcccgcgcctgcccggaggtccgcgggcaagaagcccaccaggcgcaccgagaggacctcagccggggacggcgccaactgccaccggcccgaggagcccgcggccgcggacgcgctggggacgagcgtggtggtccccccggagcccaccaaaaccaggccctgtggcaacaacagagcgagccaccgcaaggagctgcgctcctccgtgacctgcgagaagcgccgcacgcgggggctaagcaggatcgtccccaaatccaaggagttcattgagacagagtcgtcatcttcatcctcctcctcggactccgacctggagtccgagcaggaggagtaccctctgtccaaagcacagaccgtggctgcctctgcctcctccgggaatgatcagaggctgaaggaggccgctgccaacgggggcagtggtcctagggcccctgtaggctccatcaacgccaggaccaccagtgacatcgccaaggagctggaggagcagttctacacactggtcccctttggccggaacgaacttctctcccctctaaaggacagtgatgagatcaggtctctctgggtcaaaatcgacctgaccctcctgtccaggatcccagaacacctgccccaggagccaggggtattgagcgcccctgccaccaaggactctgagagcgcaccgcccagccacacctcggacacacctgcagaaaaggctttgccaaaatccaagaggaaacgcaagtgtgacaacgaagacgactacagggagatcaagaagtcccagggagagaaagacagctcttcaagactggccacctccaccagtaatactttgtctgcaaaccactgcaacatgaacatcaacagtgtggcaataccaataaataaaaatgaaaaaatgcttcggtcgcccatctcacccctctctgatgcatctaaacacaaatacaccagcgaggacttaacttcttccagccgacctaatggcaacagtttgtttacttcagcctcttccagcaaaaagcctaaggccgacagccagctgcagcctcacggcggagacctcacgaaagcagctcacaacaattctgaaaacattcccctccacaagtcacggccgcagacgaagccgtggtctccaggctccaacggccacagggactgcaagaggcagaaacttgtcttcgatgatatgcctcgcagtgccgattattttatgcaagaagctaaacgaatgaagcataaagcagatgcaatggtggaaaagtttggaaaggctttgaactatgctgaagcagcattgtcgtttatcgagtgtggaaatgcaatggaacaaggccccatggaatccaaatctccttattacctgatgtattcagaaacagtagagctcatcaggtatgctatgagactaaaaacccactcaggccccaatgccacaccagaagacaaacaactggctgcattatgttaccgatgcctggccctcctgtactggcggatgtttcgactcaaaagggaccacgctgtaaagtattcaaaagcactaatcgactatttcaagaactcatctaaagccgcccaagccccatctccgtggggggccagtggaaagagcactggaaccccatcccccatttctcccaacccctttcccggcagctccgtggggtctcagggcagcctctccaacgccagcgccctgtccccgtcgaccatcgtcagcatcccacagcgcatccaccagatggcggccaaccacgtcagcatcaccaacagcatcctgcacagctacgactactgggagatggccgacaacctggccaaggaaaaccgagaattcttcaacgacctggatctgctcatggggccggtcaccctgcacagcagcatggagcacctggtccagtactcccaacagggcctgcactggctgcggaacagcgcccacctgtcatagggacctcaccctggggccagagtgggctctggtctccacagatggctcaacgtttttggacactgtgctactgaaactcccagccacagcatttatagactgcggtgaacatttcctca

SEQ ID NO: 46 - protein, 1227 aa, Homo sapiens
MDSFDLALLQEWDLESLCVYEPDRNALRRKERERRNQETQQDDGTFNSSYSLFSEPYKTNKGDELSNRIQNTLGNYDEMKDFLTDRTNQSHLVGVPKPGVPQTPVNKIDEHFVADSRAQNQPSSICSTTTSTPAAVPVQQSKRGTMGWQKAGHPPSDGQQRATQQGSLRTLLGDGVGRQQPRAKQVCNVEVGLQTQERPPAMAAKHSSSGHCVQNFPPSLASKPSLVQQKPTAYVRPMDGQDQAPDESPKLKSSSETSVHCTSYRGVPASKPEPARAKAKLSKFSIPKQGEESRSGETNSCVEEIIREMTWLPPLSAIQAPGKVEPTKFPFPNKDSQLVSSGHNNPKKGDAEPESPDNGTSNTSMLEDDLKLSSDEEENEQQAAQRTALRALSDSAVVQQPNCRTSVPSSKGSSSSSSSGTSSSSSDSESSSGSDSETESSSSESEGSKPPHFSSPEAEPASSNKWQLDKWLNKVNPHKPPILIQNESHGSESNQYYNPVKEDVQDCGKVPDVCQPSLREKEIKSTCKEEQRPRTANKAPGSKGVKQKSPPAAVAVAVSAAAPPPAVPCAPAENAPAPARRSAGKKPTRRTERTSAGDGANCHRPEEPAAADALGTSVVVPPEPTKTRPCGNNRASHRKELRSSVTCEKRRTRGLSRIVPKSKEFIETESSSSSSSSDSDLESEQEEYPLSKAQTVAASASSGNDQRLKEAAANGGSGPRAPVGSINARTTSDIAKELEEQFYTLVPFGRNELLSPLKDSDEIRSLWVKIDLTLLSRIPEHLPQEPGVLSAPATKDSESAPPSHTSDTPAEKALPKSKRKRKCDNEDDYREIKKSQGEKDSSSRLATSTSNTLSANHCNMNINSVAIPINKNEKMLRSPISPLSDASKHKYTSEDLTSSSRPNGNSLFTSASSSKKPKADSQLQPHGGDLTKAAHNNSENIPLHKSRPQTKPWSPGSNGHRDCKRQKLVFDDMPRSADYFMQEAKRMKHKADAMVEKFGKALNYAEAALSFIECGNAMEQGPMESKSPYYLMYSETVELIRYAMRLKTHSGPNATPEDKQLAALCYRCLALLYWRMFRLKRDHAVKYSKALIDYFKNSSKAAQAPSPWGASGKSTGTPSPISPNPFPGSSVGSQGSLSNASALSPSTIVSIPQRIHQMAANHVSITNSILHSYDYWEMADNLAKENREFFNDLDLLMGPVTLHSSMEHLVQYSQQGLHWLRNSAHLS

SEQ ID NO: 47 - DNA, 4862 bp, Homo sapiens
actgggataaagcaagaagactgattttatgagcaggggtttgatacatcaaaggagattgcccaggatcaagggtgcggtgttgggggtgggttggggagggtggttagagaaggtttcactaagtgatttgggcctgaggcctgagaagatgtttaaaaagagggatcaagcacaggctaaggagaggaaagagcaggcacccaaacctctgcatggccccaatatgctccctgcagggtagtgccccctcttctggctgctcaaggcgagatctaagcttcttctaactcctgctgtcttttcatattctctgattctgggaaacgaagaattggcaggaactgaaaatgactaggaagaggacatactgggtgcccaactcttctggtggcctcgtgaatcgtggcatcgacataggcgatgacatggtttcaggacttatttataaaacctatactctccaagatggcccctggagtcagcaagagagaaatcctgaggctccagggagggcagctgtcccaccgtgggggaagtatgatgctgccttgagaaccatgattcccttccgtcccaagccgaggtttcctgccccccagcccctggacaatgctggcctgttctcctacctcaccgtgtcatggctcaccccgctcatgatccaaagcttacggagtcgcttagatgagaacaccatccctccactgtcagtccatgatgcctcagacaaaaatgtccaaaggcttcaccgcctttgggaagaagaagtctcaaggcgagggattgaaaaagcttcagtgcttctggtgatgctgaggttccagagaacaaggttgattttcgatgcacttctgggcatctgcttctgcattgccagtgtactcgggccaatattgattataccaaagatcctggaatattcagaagagcagttggggaatgttgtccatggagtgggactctgctttgccctttttctctccgaatgtgtgaagtctctgagtttctcctccagttggatcatcaaccaacgcacagccatcaggttccgagcagctgtttcctcctttgcctttgagaagctcatccaatttaagtctgtaatacacatcacctcaggagaggccatcagcttcttcaccggtgatgtaaactacctgtttgaaggggtgtgctatggacccctagtactgatcacctgcgcatcgctggtcatctgcagcatttcttcctacttcattattggatacactgcatttattgccatcttatgctatctcctggttttcccactggcggtattcatgacaagaatggctgtgaaggctcagcatcacacatctgaggtcagcgaccagcgcatccgtgtgaccagtgaagttctcacttgcattaagctgattaaaatgtacacatgggagaaaccatttgcaaaaatcattgaagacctaagaaggaaggaaaggaaactattggagaagtgcgggcttgtccagagcctgacaagtataaccttgttcatcatccccacagtggccacagcggtctgggttctcatccacacatccttaaagctgaaactcacagcgtcaatggccttcagcatgctggcctccttgaatctccttcggctgtcagtgttctttgtgcctattgcagtcaaaggtctcacgaattccaagtctgcagtgatgaggttcaagaagtttttcctccaggagagccctgttttctatgtccagacattacaagaccccagcaaagctctggtctttgaggaggccaccttgtcatggcaacagacctgtcccgggatcgtcaatggggcactggagctggagaggaacgggcatgcttctgaggggatgaccaggcctagagatgccctcgggccagaggaagaagggaacagcctgggcccagagttgcacaagatcaacctggtggtgtccaaggggatgatgttaggggtctgcggcaacacggggagtggtaagagcagcctgttgtcagccatcctggaggagatgcacttgctcgagggctcggtgggggtgcagggaagcctggcctatgtcccccagcaggcctggatcgtcagcgggaacatcagggagaacatcctcatgggaggcgcatatgacaaggcccgatacctccaggtgctccactgctgctccctgaatcgggacctggaacttctgccctttggagacatgacagagattggagagcggggcctcaacctctctggggggcagaaacagaggatcagcctggcccgcgccgtctattccgaccgtcagatctacctgctggacgaccccctgtctgctgtggacgcccacgtggggaagcacatttttgaggagtgcattaagaagacactcagggggaagacggtcgtcctggtgacccaccagctgcagtacttagaattttgtggccagatcattttgttggaaaatgggaaaatctgtgaaaatggaactcacagtgagttaatgcagaaaaaggggaaatatgcccaacttatccagaagatgcacaaggaagccacttcggacatgttgcaggacacagcaaagatagcagagaagccaaaggtagaaagtcaggctctggccacctccctggaagagtctctcaacggaaatgctgtgccggagcatcagctcacacaggaggaggagatggaagaaggctccttgagttggagggtctaccaccactacatccaggcagctggaggttacatggtctcttgcataattttcttcttcgtggtgctgatcgtcttcttaacgatcttcagcttctggtggctgagctactggttggagcagggctcggggaccaatagcagccgagagagcaatggaaccatggcagacctgggcaacattgcagacaatcctcaactgtccttctaccagctggtgtacgggctcaacgccctgctcctcatctgtgtgggggtctgctcctcagggattttcaccaaagtcacgaggaaggcatccacggccctgcacaacaagctcttcaacaaggttttccgctgccccatgagtttctttgacaccatcccaataggccggcttttgaactgcttcgcaggggacttggaacagctggaccagctcttgcccatcttttcagagcagttcctggtcctgtccttaatggtgatcgccgtcctgttgattgtcagtgtgctgtctccatatatcctgttaatgggagccataatcatggttatttgcttcatttattatatgatgttcaagaaggccatcggtgtgttcaagagactggagaactatagccggtctcctttattctcccacatcctcaattctctgcaaggcctgagctccatccatgtctatggaaaaactgaagacttcatcagccagtttaagaggctgactgatgcgcagaataactacctgctgttgtttctatcttccacacgatggatggcattgaggctggagatcatgaccaaccttgtgaccttggctgttgccctgttcgtggcttttggcatttcctccaccccctactcctttaaagtcatggctgtcaacatcgtgctgcagctggcgtccagcttccaggccactgcccggattggcttggagacagaggcacagttcacggctgtagagaggatactgcagtacatgaagatgtgtgtctcggaagctcctttacacatggaaggcacaagttgtccccaggggtggccacagcatggggaaatcatatttcaggattatcacatgaaatacagagacaacacacccaccgtgcttcacggcatcaacctgaccatccgcggccacgaagtggtgggcatcgtgggaaggacgggctctgggaagtcctccttgggcatggctctcttccgcctggtggagcccatggcaggccggattctcattgacggcgtggacatttgcagcatcggcctggaggacttgcggtccaagctctcagtgatccctcaagatccagtgctgctctcaggaaccatcagattcaacctagatccctttgaccgtcacactgaccagcagatctgggatgccttggagaggacattcctgaccaaggccatctcaaagttccccaaaaagctgcatacagatgtggtggaaaacggtggaaacttctctgtgggggagaggcagctgctctgcattgccagggctgtgcttcgcaactccaagatcatccttatcgatgaagccacagcctccattgacatggagacagacaccctgatccagcgcacaatccgtgaagccttccagggctgcaccgtgctcgtcattgcccaccgtgtcaccactgtgctgaactgtgaccacatcctggttatgggcaatgggaaggtggtagaatttgatcggccggaggtactgcggaagaagcctgggtcattgttcgcagccctcatggccacagccacttcttcactgagataaggagatgtggagacttcatggaggctggcagctgagctcagaggttcacacaggtgcagcttcgaggcccacagtctgcgaccttcttgtttggagatgagaacttctcctggaagcaggggtaaatgtagggggggtggggattgctggatggaaaccctggaataggctacttgatggctctcaagaccttagaaccccagaaccatctaagacatgggattcagtgatcatgtggttctccttttaacttacatgctgaataattttataataaggtaaaagcttatagttttctgatctgtgttagaagtgttgcaaatgctgtactgactttgtaaaatataaaactaaggaaaactc

SEQ ID NO: 48 - protein, 1382 aa, Homo sapiens
MTRKRTYWVPNSSGGLVNRGIDIGDDMVSGLIYKTYTLQDGPWSQQERNPEAPGRAAVPPWGKYDAALRTMIPFRPKPRFPAPQPLDNAGLFSYLTVSWLTPLMIQSLRSRLDENTIPPLSVHDASDKNVQRLHRLWEEEVSRRGIEKASVLLVMLRFQRTRLIFDALLGICFCIASVLGPILIIPKILEYSEEQLGNVVHGVGLCFALFLSECVKSLSFSSSWIINQRTAIRFRAAVSSFAFEKLIQFKSVIHITSGEAISFFTGDVNYLFEGVCYGPLVLITCASLVICSISSYFIIGYTAFIAILCYLLVFPLAVFMTRMAVKAQHHTSEVSDQRIRVTSEVLTCIKLIKMYTWEKPFAKIIEDLRRKERKLLEKCGLVQSLTSITLFIIPTVATAVWVLIHTSLKLKLTASMAFSMLASLNLLRLSVFFVPIAVKGLTNSKSAVMRFKKFFLQESPVFYVQTLQDPSKALVFEEATLSWQQTCPGIVNGALELERNGHASEGMTRPRDALGPEEEGNSLGPELHKINLVVSKGMMLGVCGNTGSGKSSLLSAILEEMHLLEGSVGVQGSLAYVPQQAWIVSGNIRENILMGGAYDKARYLQVLHCCSLNRDLELLPFGDMTEIGERGLNLSGGQKQRISLARAVYSDRQIYLLDDPLSAVDAHVGKHIFEECIKKTLRGKTVVLVTHQLQYLEFCGQIILLENGKICENGTHSELMQKKGKYAQLIQKMHKEATSDMLQDTAKIAEKPKVESQALATSLEESLNGNAVPEHQLTQEEEMEEGSLSWRVYHHYIQAAGGYMVSCIIFFFVVLIVFLTIFSFWWLSYWLEQGSGTNSSRESNGTMADLGNIADNPQLSFYQLVYGLNALLLICVGVCSSGIFTKVTRKASTALHNKLFNKVFRCPMSFFDTIPIGRLLNCFAGDLEQLDQLLPIFSEQFLVLSLMVIAVLLIVSVLSPYILLMGAIIMVICFIYYMMFKKAIGVFKRLENYSRSPLFSHILNSLQGLSSIHVYGKTEDFISQFKRLTDAQNNYLLLFLSSTRWMALRLEIMTNLVTLAVALFVAFGISSTPYSFKVMAVNIVLQLASSFQATARIGLETEAQFTAVERILQYMKMCVSEAPLHMEGTSCPQGWPQHGEIIFQDYHMKYRDNTPTVLHGINLTIRGHEVVGIVGRTGSGKSSLGMALFRLVEPMAGRILIDGVDICSIGLEDLRSKLSVIPQDPVLLSGTIRFNLDPFDRHTDQQIWDALERTFLTKAISKFPKKLHTDVVENGGNFSVGERQLLCIARAVLRNSKIILIDEATASIDMETDTLIQRTIREAFQGCTVLVIAHRVTTVLNCDHILVMGNGKVVEFDRPEVLRKKPGSLFAALMATATSSLR

SEQ ID NO: 49 - DNA, 2682 bp, Homo sapiens
cgagccagggagaaaggatggccggcctggcggcgcggttggtcctgctagctggggcagcggcgctggcgagcggctcccagggcgaccgtgagccggtgtaccgcgactgcgtactgcagtgcgaagagcagaactgctctgggggcgctctgaatcacttccgctcccgccagccaatctacatgagtctagcaggctggacctgtcgggacgactgtaagtatgagtgtatgtgggtcaccgttgggctctacctccaggaaggtcacaaagtgcctcagttccatggcaagtggcccttctcccggttcctgttctttcaagagccggcatcggccgtggcctcgtttctcaatggcctggccagcctggtgatgctctgccgctaccgcaccttcgtgccagcctcctcccccatgtaccacacctgtgtggccttcgcctgggtgtccctcaatgcatggttctggtccacagttttccacaccagggacactgacctcacagagaaaatggactacttctgtgcctccactgtcatcctacactcaatctacctgtgctgcgtcaggaccgtggggctgcagcacccagctgtggtcagtgccttccgggctctcctgctgctcatgctgaccgtgcacgtctcctacctgagcctcatccgcttcgactatggctacaacctggtggccaacgtggctattggcctggtcaacgtggtgtggtggctggcctggtgcctgtggaaccagcggcggctgcctcacgtgcgcaagtgcgtggtggtggtcttgctgctgcaggggctgtccctgctcgagctgcttgacttcccaccgctcttctgggtcctggatgcccatgccatctggcacatcagcaccatccctgtccacgtcctctttttcagctttctggaagatgacagcctgtacctgctgaaggaatcagaggacaagttcaagctggactgaagaccttggagcgagtctgccccagtggggatcctgcccccgccctgctggcctcccttctcccctcaacccttgagatgattttctcttttcaacttcttgaacttggacatgaaggatgtgggcccagaatcatgtggccagcccaccccctgttggccctcaccagccttggagtctgttctagggaaggcctcccagcatctgggactcgagagtgggcagcccctctacctcctggagctgaactggggtggaactgagtgtgctcttagctctaccgggaggacagctgcctgtttcctccccatcagcctcctccccacatccccagctgcctggctgggtcctgaagccctctgtctacctgggagaccagggaccacaggccttagggatacagggggtccccttctgttaccaccccccaccctcctccaggacaccactaggtggtgctggatgcttgttctttggccagccaaggttcacggcgattctccccatgggatcttgagggaccaagctgctgggattgggaaggagtttcaccctgaccattgccctagccaggttcccaggaggcctcaccatactccctttcagggccagggctccagcaagcccagggcaaggatcctgtgctgctgtctggttgagagcctgccaccgtgtgtcgggagtgtgggccaggctgagtgcataggtgacagggccgtgagcatgggcctgggtgtgtgtgagctcaggcctaggtgcgcagtgtggagacgggtgttgtcggggaagaggtgtggcttcaaagtgtgtgtgtgcagggggtgggtgtgttagcgtgggttaggggaacgtgtgtgcgcgtgctggtgggcatgtgagatgagtgactgccggtgaatgtgtccacagttgagaggttggagcaggatgagggaatcctgtcaccatcaataatcacttgtggagcgccagctctgcccaaggcgccacctgggcggacagccaggagctctccatggccaggctgcctgtgtgcatgttccctgtctggtgcccctttgcccgcctcctgcaaacctcacagggtccccacacaacagtgccctccagaagcagcccctcggaggcagaggaaggaaaatggggatggctggggctctctccatcctccttttctccttgccttcgcatggctggccttcccctccaaaacctccattcccctgctgccagcccctttgccatagcctgattttggggaggaggaaggggcgatttgagggagaaggggagaaagcttatggctgggtctggtttcttcccttcccagagggtcttactgttccagggtggccccagggcaggcaggggccacactatgcctgcgccctggtaaaggtgacccctgccatttaccagcagccctggcatgttcctgccccacaggaatagaatggagggagctccagaaactttccatcccaaaggcagtctccgtggttgaagcagactggatttttgctctgcccctgaccccttgtccctctttgagggaggggagctatgctaggactccaacctcagggactcgggtggcctgcgctagcttcttttgatactgaaaacttttaaggtgggagggtggcaagggatgtgcttaataaatcaattccaagcctcaaaaaaaaaaaaaaaaaaaaaa

SEQ ID NO: 50 - protein, 240 aa, Homo sapiens
MKDVGPESCGQPTPCWPSPALESVLGKASQHLGLESGQPLYLLELNWGGTECALSSTGRTAACFLPISLLPTSPAAWLGPEALCLPGRPGTTGLRDTGGPLLLPPPTLLQDTTRWCWMLVLWPAKVHGDSPHGILRDQAAGIGKEFHPDHCPSQVPRRPHHTPFQGQGSSKPRARILCCCLVESLPPCVGSVGQAECIGDRAVSMGLGVCELRPRCAVWRRVLSGKRCGFKVCVCRGWVC

SEQ ID NO: 51 - DNA, 1194 bp, Homo sapiens
gatgtacactctgaagtgagcacattcctgttggcaggacatgacaccttggcagcaagcatctcctggatcctttactgcctggctctgaaccctgagcatcaagagagatgccgggaggaggtcaggggcatcctgggggatgggtcttctatcacttgggaccagctgggtgagatgtcgtacaccacaatgtgcatcaaggagacgtgccgattgattcctgcagtcccgtccatttccagagatctcagcaagccacttaccttcccagatggatgcacattgcctgcagggatcaccgtggttcttagtatttggggtcttcaccacaaccctgctgtctggaaaaacccaaaggtctttgaccccttgaggttctctcaggagaattctgatcagagacacccctatgcctacttaccattctcagctggatcaaggaactgcattgggcaggagtttgccatgattgagttaaaggtaaccattgccttgattctgctccacttcagagtgactccagaccccaccaggcctcttactttccccaaccattttatcctcaagcccaagaatgggatgtatttgcacctgaagaaactctctgaatgttagatctcagggtacaatgattaaacgtactttgtttttcgaagttaaatttacagctaatgatccaagcagatagaaagggatcaatgtatggtgggaggattggaggttggtgggataggggtctctgtgaagagatccaaaatcatttctaggtacacagtgtgtcagctagatctgtttctatataactttgggagattttcagatcttttctgttaaactttcactactattaatgctgtatacaccaatagactttcatatattttctgttgtttttaaaatagttttcagaattatgcaagtaataagtgcatgtatgctcactgtcaaaaattcccaacactagaaaatcatgtagaataaaaattttaaatctcacttcacttagccgacattccatgccctgaccaatcctactgcttttcctaaaaacagaataatttggtgtgcattctttcagactttttcctatacattttatatgtagaaatgtagcaatgtatttgtatagatgtgatcattcctatattgttattgatttttttcacttaataaaaattcaccttattcctt

SEQ ID NO: 52 - protein, 146 aa, Homo sapiens [sequence omitted]

SEQ ID NO: 53 - DNA, 1533 bp, Homo sapiens
atgcccctgtccctgggagccgagatgtgggggcctgaggcctggctgctgctgctgctactgctggcatcatttacaggccggtgccccgcgggtgagctggagacctcagacgtggtaactgtggtgctgggccaggacgcaaaactgccctgcttctaccgaggggactccggcgagcaagtggggcaagtggcatgggctcgggtggacgcgggcgaaggcgcccaggaactagcgctactgcactccaaatacgggcttcatgtgagcccggcttacgagggccgcgtggagcagccgccgcccccacgcaaccccctggacggctcagtgctcctgcgcaacgcagtgcaggcggatgagggcgagtacgagtgccgggtcagcaccttccccgccggcagcttccaggcgcggctgcggctccgagtgctggtgcctcccctgccctcactgaatcctggtccagcactagaagagggccagggcctgaccctggcagcctcctgcacagctgagggcagcccagcccccagcgtgacctgggacacggaggtcaaaggcacaacgtccagccgttccttcaagcactcccgctctgctgccgtcacctcagagttccacttggtgcctagccgcagcatgaatgggcagccactgacttgtgtggtgtcccatcctggcctgctccaggaccaaaggatcacccacatcctccacgtgtccttccttgctgaggcctctgtgaggggccttgaagaccaaaatctgtggcacattggcagagaaggagctatgctcaagtgcctgagtgaagggcagccccctccctcatacaactggacacggctggatgggcctctgcccagtggggtacgagtggatggggacactttgggctttcccccactgaccactgagcacagcggcatctacgtctgccatgtcagcaatgagttctcctcaagggattctcaggtcactgtggatgttcttgacccccaggaagactctgggaagcaggtggacctagtgtcagcctcggtggtggtggtgggtgtgatcgccgcactcttgttctgccttctggtggtggtggtggtgctcatgtcccgataccatcggcgcaaggcccagcagatgacccagaaatatgaggaggagctgaccctgaccagggagaactccatccggaggctgcattcccatcacacggaccccaggagccagccggaggagagtgtagggctgagagccgagggccaccctgatagtctcaaggacaacagtagctgctctgtgatgagtgaagagcccgagggccgcagttactccacgctgaccacggtgagggagatagaaacacagactgaactgctgtctccaggctctgggcgggccgaggaggaggaagatcaggatgaaggcatcaaacaggccatgaaccattttgttcaggagaatgggaccctacgggccaagcccacgggcaatggcatctacatcaatgggcggggacacctggtctga

SEQ ID NO: 54 - protein, 510 aa, Homo sapiens
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 55 - DNA, 2642 bp, Homo sapiens
aaggaggcggcctccgggaaaagcgaccgcaggactcctgagagcagcctccatgaggccctggaccagtgcatgaccgccctggacctcttcctcaccaaccagttctcagaagcactcagctacctcaagcccagaaccaaggaaagcatgtaccactcactgacatatgccaccatcctggagatgcaggccatgatgacctttgaccctcaggacatcctgcttgccggcaacatgatgaaggaggcacagatgctgtgtcagaggcaccggaggaagtcttctgtaacagattccttcagcagcctggtgaaccgccccacgctgggccaattcactgaagaagaaatccacgctgaggtctgctatgcagagtgcctgctgcagcgagcagccctgaccttcctgcaggacgagaacatggtgagcttcatcaaaggcggcatcaaagttcgaaacagctaccagacctacaaggagctggacagccttgttcagtcctcacaatactgcaagggtgagaaccacccgcactttgaaggaggagtgaagcttggtgtaggggccttcaacctgacactgtccatgcttcctactaggatcctgaggctgttggagtttgtggggttttcaggaaacaaggactatgggctgctgcagctggaggagggagcgtcagggcacagcttccgctctgtgctctgtgtcatgctcctgctgtgctaccacaccttcctcaccttcgtgctcggtactgggaacgtcaacatcgaggaggccgagaagctcttgaagccctacctgaaccggtaccctaagggtgccatcttcctgttctttgcagggaggattgaagtcattaaaggcaacattgatgcagccatccggcgtttcgaggagtgctgtgaggcccagcagcactggaagcagtttcaccacatgtgctactgggagctgatgtggtgcttcacctacaagggccagtggaagatgtcctacttctacgccgacctgctcagcaaggagaactgctggtccaaggccacctacatttacatgaaggccgcctacctcagcatgtttgggaaggaggaccacaagccgttcggggacgacgaagtggaattatttcgagctgtgccaggcctgaagctcaagattgctgggaaatctctacccacagagaagtttgccatccggaagtcccggcgctacttctcctccaaccctatctcgctgccagtgcctgctctggaaatgatgtacatctggaacggctacgccgtgattgggaagcagccgaaactcacggatgggatacttgagattatcactaaggctgaagagatgctggagaaaggcccagagaacgagtactcagtggatgacgagtgcttggtgaaattgttgaaaggcctgtgtctgaaatacctgggccgtgtccaggaggccgaggagaattttaggagcatctctgccaatgaaaagaagattaaatatgaccactacttgatcccaaacgccctgctggagctggccctgctgcttatggagcaagacagaaacgaagaggccatcaaacttttggaatctgccaagcaaaactacaagaattactccatggagtcaaggacacactttcgaatccaggcagccacactccaagccaagtcttccctagagaacagcagcagatccatggtctcatcagtgtccttgtagctttgtgcagcagttccgggctggaagacagagacagctggacagagctcctgaaaacatttcaaaataccccctccccctgccctgccctgcctttggggtccaccggcactccagttggatggcacaacatagtgtatccgtgcagaagccgagctggcattttcaccagtgtagccaagggcctttgccaagggcagagcaggtggagccctctgcctgccctatcacacatacgggtacttgcttttcactgtgatgtttaagagaatgtatgaacagtttacattttccttagaaatacattgatgggatcacagttggctttaaaaaccaacaacaatcaaccacctgtaagtctttgtcttcacctattatcatctggaggtaaatctctttatatgatgatgccaaagggcaaattgcttttcaaattcagcaagttctcagcttgtgtgacggaaggtccttcagaggacctgaggaatgcctgggagaggctaagcctcaggcttcaatgcttctggggttgggcatgaggatgtacacagacacccactaccttactactcacacttcatttcactccttttgtaaatttccaatttaaaaatcaagcacgtctttttagtgagataaaatctgagctcttctgtagaaaaatcaatctctaccagtagaaaatgccagggcttgatggaagagctgtgtagccctttctatgccaaagccaggaaatttggggggcaggaggaggttctcagaatccagtctgtatctttgctgtatgccaaactgaaaccactgggaataatttatgaaacataaaaatcttctgtacttcactccaaggtacatttgcttactgacagcatttttgttaaaactgttattcttgaaaaaaaaaaaaaaaaaaa

SEQ ID NO: 56 - protein, 550 aa, Homo sapiens
MTALDLFLTNQFSEALSYLKPRTKESMYHSLTYATILEMQAMMTFDPQDILLAGNMMKEAQMLCQRHRRKSSVTDSFSSLVNRPTLGQFTEEEIHAEVCYAECLLQRAALTFLQDENMVSFIKGGIKVRNSYQTYKELDSLVQSSQYCKGENHPHFEGGVKLGVGAFNLTLSMLPTRILRLLEFVGFSGNKDYGLLQLEEGASGHSFRSVLCVMLLLCYHTFLTFVLGTGNVNIEEAEKLLKPYLNRYPKGAIFLFFAGRIEVIKGNIDAAIRRFEECCEAQQHWKQFHHMCYWELMWCFTYKGQWKMSYFYADLLSKENCWSKATYIYMKAAYLSMFGKEDHKPFGDDEVELFRAVPGLKLKIAGKSLPTEKFAIRKSRRYFSSNPISLPVPALEMMYIWNGYAVIGKQPKLTDGILEIITKAEEMLEKGPENEYSVDDECLVKLLKGLCLKYLGRVQEAEENFRSISANEKKIKYDHYLIPNALLELALLLMEQDRNEEAIKLLESAKQNYKNYSMESRTHFRIQAATLQAKSSLENSSRSMVSSVSL

SEQ ID NO: 57 - DNA, 927 bp, Homo sapiens
atgcctggggggtgctcccggggccccgccgccggggacgggcgtctgcggctggcgcgactagcgctggtactcctgggctgggtctcctcgtcttctcccacctcctcggcatcctccttctcctcctcggcgccgttcctggcttccgccgtgtccgcccagcccccgctgccggaccagtgccccgcgctgtgcgagtgctccgaggcagcgcgcacagtcaagtgcgttaaccgcaatctgaccgaggtgcccacggacctgcccgcctacgtgcgcaacctcttccttaccggcaaccagctggccagcaaccacttcctttacctgccgcgggatgtgctggcccaactgcccagcctcaggcacctggacttaagtaataattcgctggtgagcctgacctacgtgtccttccgcaacctgacacatctagaaagcctccacctggaggacaatgccctcaaggtccttcacaatggcaccctggctgagttgcaaggtctaccccacattagggttttcctggacaacaatccctgggtctgcgactgccacatggcagacatggtgacctggctcaaggaaacagaggtagtgcagggcaaagaccggctcacctgtgcatatccggaaaaaatgaggaatcgggtcctcttggaactcaacagtgctgacctggactgtgacccgattcttcccccatccctgcaaacctcttatgtcttcctgggtattgttttagccctgataggcgctattttcctcctggttttgtatttgaaccgcaaggggataaaaaagtggatgcataacatcagagatgcctgcagggatcacatggaagggtatcattacagatatgaaatcaatgcggaccccagattaacaaacctcagttctaactcggatgtcctcgagtga

SEQ ID NO: 58 - protein, 308 aa, Homo sapiens
MPGGCSRGPAAGDGRLRLARLALVLLGWVSSSSPTSSASSFSSSAPFLASAVSAQPPLPDQCPALCECSEAARTVKCVNRNLTEVPTDLPAYVRNLFLTGNQLASNHFLYLPRDVLAQLPSLRHLDLSNNSLVSLTYVSFRNLTHLESLHLEDNALKVLHNGTLAELQGLPHIRVFLDNNPWVCDCHMADMVTWLKETEVVQGKDRLTCAYPEKMRNRVLLELNSADLDCDPILPPSLQTSYVFLGIVLALIGAIFLLVLYLNRKGIKKWMHNIRDACRDHMEGYHYRYEINADPRLTNLSSNSDVLE

SEQ ID NO: 59 - DNA, 1362 bp, Homo sapiens
atgggggaaaatgatccgcctgctgttgaagcccccttctcattccgatcgctttttggccttgatgatttgaaaataagtcctgttgcaccagatgcagatgctgttgctgcacagatcctgtcactgctgccattgaagttttttccaatcatcgtcattgggatcattgcattgatattagcactggccattggtctgggcatccacttcgactgctcagggaagtacagatgtcgctcatcctttaagtgtatcgagctgatagctcgatgtgacggagtctcggattgcaaagacggggaggacgagtaccgctgtgtccgggtgggtggtcagaatgccgtgctccaggtgttcacagctgcttcgtggaagaccatgtgctccgatgactggaagggtcactacgcaaatgttgcctgtgcccaactgggtttcccaagctatgtgagttcagataacctcagagtgagctcgctggaggggcagttccgggaggagtttgtgtccatcgatcacctcttgccagatgacaaggtgactgcattacaccactcagtatatgtgagggagggatgtgcctctggccacgtggttaccttgcagtgcacagcctgtggtcatagaaggggctacagctcacgcatcgtgggtggaaacatgtccttgctctcgcagtggccctggcaggccagccttcagttccagggctaccacctgtgcgggggctctgtcatcacgcccctgtggatcatcactgctgcacactgtgtttatgacttgtacctccccaagtcatggaccatccaggtgggtctagtttccctgttggacaatccagccccatcccacttggtggagaagattgtctaccacagcaagtacaagccaaagaggctgggcaatgacatcgcccttatgaagctggccgggccactcacgttcaatgaaatgatccagcctgtgtgcctgcccaactctgaagagaacttccccgatggaaaagtgtgctggacgtcaggatggggggccacagaggatggaggtgacgcctcccctgtcctgaaccacgcggccgtccctttgatttccaacaagatctgcaaccacagggacgtgtacggtggcatcatctccccctccatgctctgcgcgggctacctgacgggtggcgtggacagctgccagggggacagcggggggcccctggtgtgtcaagagaggaggctgtggaagttagtgggagcgaccagctttggcatcggctgcgcagaggtgaacaagcctggggtgtacacccgtgtcacctccttcctggactggatccacgagcagatggagagagacctaaaaacctga

SEQ ID NO: 60 - protein, 453 aa, Homo sapiens
MGENDPPAVEAPFSFRSLFGLDDLKISPVAPDADAVAAQILSLLPLKFFPIIVIGIIALILALAIGLGIHFDCSGKYRCRSSFKCIELIARCDGVSDCKDGEDEYRCVRVGGQNAVLQVFTAASWKTMCSDDWKGHYANVACAQLGFPSYVSSDNLRVSSLEGQFREEFVSIDHLLPDDKVTALHHSVYVREGCASGHVVTLQCTACGHRRGYSSRIVGGNMSLLSQWPWQASLQFQGYHLCGGSVITPLWIITAAHCVYDLYLPKSWTIQVGLVSLLDNPAPSHLVEKIVYHSKYKPKRLGNDIALMKLAGPLTFNEMIQPVCLPNSEENFPDGKVCWTSGWGATEDGGDASPVLNHAAVPLISNKICNHRDVYGGIISPSMLCAGYLTGGVDSCQGDSGGPLVCQERRLWKLVGATSFGIGCAEVNKPGVYTRVTSFLDWIHEQMERDLKT

SEQ ID NO: 61 - DNA, 3229 bp, Homo sapiens
aacagaactgcaacggagagactcaagatgattccctttttacccatgttttctctactattgctgcttattgttaaccctataaacgccaacaatcattatgacaagatcttggctcatagtcgtatcaggggtcgggaccaaggcccaaatgtctgtgcccttcaacagattttgggcaccaaaaagaaatacttcagcacttgtaagaactggtataaaaagtccatctgtggacagaaaacgactgttttatatgaatgttgccctggttatatgagaatggaaggaatgaaaggctgcccagcagttttgcccattgaccatgtttatggcactctgggcatcgtgggagccaccacaacgcagcgctattctgacgcctcaaaactgagggaggagatcgagggaaagggatccttcacttactttgcaccgagtaatgaggcttgggacaacttggattctgatatccgtagaggtttggagagcaacgtgaatgttgaattactgaatgctttacatagtcacatgattaataagagaatgttgaccaaggacttaaaaaatggcatgattattccttcaatgtataacaatttggggcttttcattaaccattatcctaatggggttgtcactgttaattgtgctcgaatcatccatgggaaccagattgcaacaaatggtgttgtccatgtcattgaccgtgtgcttacacaaattggtacctcaattcaagacttcattgaagcagaagatgacctttcatcttttagagcagctgccatcacatcggacatattggaggcccttggaagagacggtcacttcacactctttgctcccaccaatgaggcttttgagaaacttccacgaggtgtcctagaaaggttcatgggagacaaagtggcttccgaagctcttatgaagtaccacatcttaaatactctccagtgttctgagtctattatgggaggagcagtctttgagacgctggaaggaaatacaattgagataggatgtgacggtgacagtataacagtaaatggaatcaaaatggtgaacaaaaaggatattgtgacaaataatggtgtgatccatttgattgatcaggtcctaattcctgattctgccaaacaagttattgagctggctggaaaacagcaaaccaccttcacggatcttgtggcccaattaggcttggcatctgctctgaggccagatggagaatacactttgctggcacctgtgaataatgcattttctgatgatactctcagcatggttcagcgcctccttaaattaattctgcagaatcacatattgaaagtaaaagttggccttaatgagctttacaacgggcaaatactggaaaccatcggaggcaaacagctcagagtcttcgtatatcgtacagctgtctgcattgaaaattcatgcatggagaaagggagtaagcaagggagaaacggtgcgattcacatattccgcgagatcatcaagccagcagagaaatccctccatgaaaagttaaaacaagataagcgctttagcaccttcctcagcctacttgaagctgcagacttgaaagagctcctgacacaacctggagactggacattatttgtgccaaccaatgatgcttttaagggaatgactagtgaagaaaaagaaattctgatacgggacaaaaatgctcttcaaaacatcattctttatcacctgacaccaggagttttcattggaaaaggatttgaacctggtgttactaacattttaaagaccacacaaggaagcaaaatctttctgaaagaagtaaatgatacacttctggtgaatgaattgaaatcaaaagaatctgacatcatgacaacaaatggtgtaattcatgttgtagataaactcctctatccagcagacacacctgttggaaatgatcaactgctggaaatacttaataaattaatcaaatacatccaaattaagtttgttcgtggtagcaccttcaaagaaatccccgtgactgtctatacaactaaaattataaccaaagttgtggaaccaaaaattaaagtgattgaaggcagtcttcagcctattatcaaaactgaaggacccacactaacaaaagtcaaaattgaaggtgaacctgaattcagactgattaaagaaggtgaaacaataactgaagtgatccatggagagccaattattaaaaaatacaccaaaatcattgatggagtgcctgtggaaataactgaaaaagagacacgagaagaacgaatcattacaggtcctgaaataaaatacactaggatttctactggaggtggagaaacagaagaaactctgaagaaattgttacaagaagaggtcaccaaggtcaccaaattcattgaaggtggtgatggtcatttatttgaagatgaagaaattaaaagactgcttcagggagacacacccgtgaggaagttgcaagccaacaaaaaagttcaaggttctagaagacgattaagggaaggtcgttctcagtgaaaatccaaaaaccagaaaaaaatgtttatacaaccctaagtcaataacctgaccttagaaaattgtgagagccaagttgacttcaggaactgaaacatcagcacaaagaagcaatcatcaaataattctgaacacaaatttaatatttttttttctgaatgagaaacatgagggaaattgtggagttagcctcctgtggtaaaggaattgaagaaaatataacaccttacaccctttttcatcttgacattaaaagttctggctaactttggaatccattagagaaaaatccttgtcaccagattcattacaattcaaatcgaagagttgtgaactgttatcccattgaaaagaccgagccttgtatgtatgttatggatacataaaatgcacgcaagccattatctctccatgggaagctaagttataaaaataggtgcttggtgtacaaaactttttatatcaaaaggctttgcacatttctatatgagtgggtttactggtaaattatgttattttttacaactaattttgtactctcagaatgtttgtcatatgcttcttgcaatgcatattttttaatctcaaacgtttcaataaaaccatttttcagatataaagagaattacttcaaattgagtaattcagaaaaactcaagatttaagttaaaaagtggtttggacttgggaa

SEQ ID NO: 62 - protein, 836 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERFMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMVQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 63 - DNA, 3737 bp, Homo sapiens
ggcgtccgcgcacacctccccgcgccgccgccgccaccgcccgcactccgccgcctctgcccgcaaccgctgagccatccatgggggtcgcgggccgcaaccgtcccggggcggcctgggcggtgctgctgctgctgctgctgctgccgccactgctgctgctggcgggggccgtcccgccgggtcggggccgtgccgcggggccgcaggaggatgtagatgagtgtgcccaagggctagatgactgccatgccgacgccctgtgtcagaacacacccacctcctacaagtgctcctgcaagcctggctaccaaggggaaggcaggcagtgtgaggacatcgatgaatgtggaaatgagctcaatggaggctgtgtccatgactgtttgaatattccaggcaattatcgttgcacttgttttgatggcttcatgttggctcatgacggtcataattgtcttgatgtggacgagtgcctggagaacaatggcggctgccagcatacctgtgtcaacgtcatggggagctatgagtgctgctgcaaggaggggtttttcctgagtgacaatcagcacacctgcattcaccgctcggaagagggcctgagctgcatgaataaggatcacggctgtagtcacatctgcaaggaggccccaaggggcagcgtcgcctgtgagtgcaggcctggttttgagctggccaagaaccagagagactgcatcttgacctgtaaccatgggaacggtgggtgccagcactcctgtgacgatacagccgatggcccagagtgcagctgccatccacagtacaagatgcacacagatgggaggagctgccttgagcgagaggacactgtcctggaggtgacagagagcaacaccacatcagtggtggatggggataaacgggtgaaacggcggctgctcatggaaacgtgtgctgtcaacaatggaggctgtgaccgcacctgtaaggatacttcgacaggtgtccactgcagttgtcctgttggattcactctccagttggatgggaagacatgtaaagatattgatgagtgccagacccgcaatggaggttgtgatcatttctgcaaaaacatcgtgggcagttttgactgcggctgcaagaaaggatttaaattattaacagatgagaagtcttgccaagatgtggatgagtgctctttggataggacctgtgaccacagctgcatcaaccaccctggcacatttgcttgtgcttgcaaccgagggtacaccctgtatggcttcacccactgtggagacaccaatgagtgcagcatcaacaacggaggctgtcagcaggtctgtgtgaacacagtgggcagctatgaatgccagtgccaccctgggtacaagctccactggaataaaaaagactgtgtggaagtgaaggggctcctgcccacaagtgtgtcaccccgtgtgtccctgcactgcggtaagagtggtggaggagacgggtgcttcctcagatgtcactctggcattcacctctcttcagatgtcaccaccatcaggacaagtgtaacctttaagctaaatgaaggcaagtgtagtttgaaaaatgctgagctgtttcccgagggtctgcgaccagcactaccagagaagcacagctcagtaaaagagagcttccgctacgtaaaccttacatgcagctctggcaagcaagtcccaggagcccctggccgaccaagcacccctaaggaaatgtttatcactgttgagtttgagcttgaaactaaccaaaaggaggtgacagcttcttgtgacctgagctgcatcgtaaagcgaaccgagaagcggctccgtaaagccatccgcacgctcagaaaggccgtccacagggagcagtttcacctccagctctcaggcatgaacctcgacgtggctaaaaagcctcccagaacatctgaacgccaggcagagtcctgtggagtgggccagggtcatgcagaaaaccaatgtgtcagttgcagggctgggacctattatgatggagcacgagaacgctgcattttatgtccaaatggaaccttccaaaatgaggaaggacaaatgacttgtgaaccatgcccaagaccaggaaattctggggccctgaagaccccagaagcttggaatatgtctgaatgtggaggtctgtgtcaacctggtgaatattctgcagatggctttgcaccttgccagctctgtgccctgggcacgttccagcctgaagctggtcgaacttcctgcttcccctgtggaggaggccttgccaccaaacatcagggagctacttcctttcaggactgtgaaaccagagttcaatgttcacctggacatttctacaacaccaccactcaccgatgtattcgttgcccagtgggaacataccagcctgaatttggaaaaaataattgtgtttcttgcccaggaaatactacgactgactttgatggctccacaaacataacccagtgtaaaaacagaagatgtggaggggagctgggagatttcactgggtacattgaatccccaaactacccaggcaattacccagccaacaccgagtgtacgtggaccatcaacccaccccccaagcgccgcatcctgatcgtggtccctgagatcttcctgcccatagaggacgactgtggggactatctggtgatgcggaaaacctcttcatccaattctgtgacaacatatgaaacctgccagacctacgaacgccccatcgccttcacctccaggtcaaagaagctgtggattcagttcaagtccaatgaagggaacagcgctagagggttccaggtcccatacgtgacatatgatgaggactaccaggaactcattgaagacatagttcgagatggcaggctctatgcatctgagaaccatcaggaaatacttaaggataagaaacttatcaaggctctgtttgatgtcctggcccatccccagaactatttcaagtacacagcccaggagtcccgagagatgtttccaagatcgttcatccgattgctacgttccaaagtgtccaggtttttgagaccttacaaatgactcagcccacgtgccactcaatacaaatgttctgctatagggttggtgggacagagctgtcttccttctgcatgtcagcacagtcgggtattgctgcctcccgtatcagtgactcattagagttcaatttttatagataatacagatattttggtaaattgaacttggtttttctttcccagcatcgtggatgtagactgagaatggctttgagtggcatcagcttctcactgctgtgggcggatgtcttggatagatcacgggctggctgagctggactttggtcagcctaggtgagactcacctgtccttctggggtcttactcctcctcaaggagtctgtagtggaaaggaggccacagaataagctgcttattctgaaacttcagcttcctctagcccggccctctctaagggagccctctgcactcgtgtgcaggctctgaccaggcagaacaggcaagaggggagggaaggagacccctgcaggctccctccacccaccttgagacctgggaggactcagtttctccacagccttctccagcctgtgtgatacaagtttgatcccaggaacttgagttctaagcagtgctcgtgaaaaaaaaaagcagaaagaattagaaataaataaaaactaagcacttctggagacat

SEQ ID NO: 64 - protein, 999 aa, Homo sapiens
MGVAGRNRPGAAWAVLLLLLLLPPLLLLAGAVPPGRGRAAGPQEDVDECAQGLDDCHADALCQNTPTSYKCSCKPGYQGEGRQCEDIDECGNELNGGCVHDCLNIPGNYRCTCFDGFMLAHDGHNCLDVDECLENNGGCQHTCVNVMGSYECCCKEGFFLSDNQHTCIHRSEEGLSCMNKDHGCSHICKEAPRGSVACECRPGFELAKNQRDCILTCNHGNGGCQHSCDDTADGPECSCHPQYKMHTDGRSCLEREDTVLEVTESNTTSVVDGDKRVKRRLLMETCAVNNGGCDRTCKDTSTGVHCSCPVGFTLQLDGKTCKDIDECQTRNGGCDHFCKNIVGSFDCGCKKGFKLLTDEKSCQDVDECSLDRTCDHSCINHPGTFACACNRGYTLYGFTHCGDTNECSINNGGCQQVCVNTVGSYECQCHPGYKLHWNKKDCVEVKGLLPTSVSPRVSLHCGKSGGGDGCFLRCHSGIHLSSDVTTIRTSVTFKLNEGKCSLKNAELFPEGLRPALPEKHSSVKESFRYVNLTCSSGKQVPGAPGRPSTPKEMFITVEFELETNQKEVTASCDLSCIVKRTEKRLRKAIRTLRKAVHREQFHLQLSGMNLDVAKKPPRTSERQAESCGVGQGHAENQCVSCRAGTYYDGARERCILCPNGTFQNEEGQMTCEPCPRPGNSGALKTPEAWNMSECGGLCQPGEYSADGFAPCQLCALGTFQPEAGRTSCFPCGGGLATKHQGATSFQDCETRVQCSPGHFYNTTTHRCIRCPVGTYQPEFGKNNCVSCPGNTTTDFDGSTNITQCKNRRCGGELGDFTGYIESPNYPGNYPANTECTWTINPPPKRRILIVVPEIFLPIEDDCGDYLVMRKTSSSNSVTTYETCQTYERPIAFTSRSKKLWIQFKSNEGNSARGFQVPYVTYDEDYQELIEDIVRDGRLYASENHQEILKDKKLIKALFDVLAHPQNYFKYTAQESREMFPRSFIRLLRSKVSRFLRPYK

SEQ ID NO: 65 - DNA, 2881 bp, Homo sapiens
atgaggctcctccgcagacgccacatgcccctgcgcctggccatggtgggctgcgcctttgtgctcttcctcttcctcctgcatagggatgtgagcagcagagaggaggccacagagaagccgtggctgaagtccctggtgagccggaaggatcacgtcctggacctcatgctggaggccatgaacaaccttagagattcaatgcccaagctccaaatcagggctccagaagcccagcagactctgttctccataaaccagtcctgcctccctgggttctataccccagctgaactgaagcccttctgggaacggccaccacaggaccccaatgcccctggggcagatggaaaagcatttcagaagagcaagtggacccccctggagacccaggaaaaggaagaaggctataagaagcactgtttcaatgcctttgccagcgaccggatctccctgcagaggtccctggggccagacacccgaccacctgagtgtgtggaccagaagttccggcgctgccccccactggccaccaccagcgtgatcattgtgttccacaacgaagcctggtccacactgctgcgaacagtgtacagcgtcctacacaccacccctgccatcttgctcaaggagatcatactggtggatgatgccagcacagaggagcacctaaaggagaagctggagcagtacgtgaagcagctgcaggtggtgagggtggtgcggcaggaggagcggaaggggttgatcaccgcccggctgctgggggccagcgtggcacaggcggaggtgctcacgttcctggatgcccactgtgagtgcttccacggctggctggagcccctcctggctcgaatcgctgaggacaagacagtggtggtgagcccagacatcgtcaccatcgaccttaatacttttgagttcgccaagcccgtccagaggggcagagtccatagccgaggcaactttgactggagcctgaccttcggctgggaaacacttcctccacatgagaagcagaggcgcaaggatgaaacataccccatcaaatccccgacgtttgctggtggcctcttctccatccccaagtcctactttgagcacatcggtacctatgataatcagatggagatctggggaggggagaacgtggaaatgtccttccgggtgtggcagtgtgggggccagctggagatcatcccctgctctgtcgtaggccatgtgttccggaccaagagcccccacaccttccccaagggcactagtgtcattgctcgcaatcaagtgcgcctggcagaggtctggatggacagctacaagaagattttctataggagaaatctgcaggcagcaaagatggcccaagagaaatccttcggtgacatttcggaacgactgcagctgagggaacaactgcactgtcacaacttttcctggtacctgcacaatgtctacccagagatgtttgttcctgacctgacgcccaccttctatggtgccatcaagaacctcggcaccaaccaatgcctggatgtgggtgagaacaaccgcggggggaagcccctcatcatgtactcctgccacggccttggcggcaaccagtactttgagtacacaactcagagggaccttcgccacaacatcgcaaagcagctgtgtctacatgtcagcaagggtgctctgggccttgggagctgtcacttcactggcaagaatagccaggtccccaaggacgaggaatgggaattggcccaggatcagctcatcaggaactcaggatctggtacctgcctgacatcccaggacaaaaagccagccatggccccctgcaatcccagtgacccccatcagttgtggctctttgtctaggacccagatcatccccagagagagcccccacaagctcctcaggaaacaggattgctgatgtctgggaacctgatcaccagcttctctggaggccgtaaagatggatttctaaacccactgggtggcaaggcaggaccttcctaatccttgcaacaacattgggcccattttctttccttcacaccgatggaagagaccattaggacatatatttagcctagcgttttcctgttctagaaatagaggctcccaaagtagggaaggcagctgggggagggttcagggcagcaatgctgagttcaagaaaagtacttcaggctgggcacagtggctcatgcctgaaatcctagcactttgggaagacaatgtgggagaatggcttgagcccaggagttcaagaccggcctgagcaacatagtgaggatcccatctctacgcccaccctccccccggcaaaaaaaaaagctgggtatggtggcttatgcctgtagtcgcagctactcagaaggctgaggtgggaggattgcttgttccccggaggttgaagctacagtgagccttgattgtgtcactgcactccagcctgggcaacaggtaagactctgtctcaaaaaaaaaacaaaaaagaagaagaaaagtacttctacagccatgtcctattccttgatcatccaaagcacctgcagagtccagtgaaatgatatattctggctgggcacagtggctcacacctgtaatcctagcactttgggaggccaaggcaggtggatcacctgaggtcagaagtttgaaaccagcctggactacatggtgaaactccatctctactaaaagtacaaaaattagctgggcatgatggcacgcacctgcagtcccagctacttgggaggctgaggcaggagaatcactcgaacccaggaggcagaggttgcagtgagccaagacagcaccattgcaccccagcctgagcaacaagagcgaaactccatctcaggaaaaaaaaaaaaaaaaa

SEQ ID NO: 66 - protein, 622 aa, Homo sapiens
MRLLRRRHMPLRLAMVGCAFVLFLFLLHRDVSSREEATEKPWLKSLVSRKDHVLDLMLEAMNNLRDSMPKLQIRAPEAQQTLFSINQSCLPGFYTPAELKPFWERPPQDPNAPGADGKAFQKSKWTPLETQEKEEGYKKHCFNAFASDRISLQRSLGPDTRPPECVDQKFRRCPPLATTSVIIVFHNEAWSTLLRTVYSVLHTTPAILLKEIILVDDASTEEHLKEKLEQYVKQLQVVRVVRQEERKGLITARLLGASVAQAEVLTFLDAHCECFHGWLEPLLARIAEDKTVVVSPDIVTIDLNTFEFAKPVQRGRVHSRGNFDWSLTFGWETLPPHEKQRRKDETYPIKSPTFAGGLFSIPKSYFEHIGTYDNQMEIWGGENVEMSFRVWQCGGQLEIIPCSVVGHVFRTKSPHTFPKGTSVIARNQVRLAEVWMDSYKKIFYRRNLQAAKMAQEKSFGDISERLQLREQLHCHNFSWYLHNVYPEMFVPDLTPTFYGAIKNLGTNQCLDVGENNRGGKPLIMYSCHGLGGNQYFEYTTQRDLRHNIAKQLCLHVSKGALGLGSCHFTGKNSQVPKDEEWELAQDQLIRNSGSGTCLTSQDKKPAMAPCNPSDPHQLWLFV

SEQ ID NO: 67 - DNA, 10011 bp, Homo sapiens
ttcctccgcgaaggctcctttgatattaatagtgttggtgtcttgaaactgacgtaatgcgcggagactgaggtcctgacaagcgataacatttctgataaagacccgatcttactgcaatctctagcgtcctcttttttggtgctgctggtttctccagacctcgcgtcctctcgattgctctctcgccttcctatttcttttttttttttttaaacaaaaaacaacaccccctcccctctcccacccggcaccgggcacatccttgctctatttcctttctctttctctctctctctctctcttttttaataagggtgggggagggaaaggggggggaggcaggaaagacctttttctctcccccccgcaataatccaagatcaactctgcaaacaacagaagacggttcatggctttggccgccgcgccaccatctttcgggctgccgagggtgttcttgacgattaatcaacagatgtacagatcagctctcaaaatgtcttctgtgtcttctgagcgtcttctaagacaattgcattagcctcctgctagttgactaatagaattaataattgtaaaaagcactctaaagccacatgccttatgaagtcaatgctgggtatgattttacaaatatggtccggaaaaagaacccccctctgagaaacgttgcaagtgaaggcgagggccagatcctggagcctataggtacagaaagcaaggtatctggaaagaacaaagaattctctgcagatcagatgtcagaaaatacggatcagagtgatgctgcagaactaaatcataaggaggaacatagcttgcatgttcaagatccatcttctagcagtaagaaggacttgaaaagcgcagttctgagtgagaaggctggcttcaattatgaaagccccagtaagggaggaaactttccctcctttccgcatgatgaggtgacagacagaaatatgttggctttctcatttccagctgctgggggagtctgtgagcccttgaagtctccgcaaagagcagaggcagatgaccctcaagatatggcctgcaccccctcaggggactcactggagacaaaggaagatcagaagatgtcaccaaaggctacagaggaaacagggcaagcacagagtggtcaagccaattgtcaaggtttgagcccagtttcagtggcctcaaaaaacccacaagtgccttcagatgggggtgtaagactgaataaatccaaaactgacttactggtgaatgacaacccagacccggcacctctgtctccagagcttcaggactttaaatgcaatatctgtggatatggttactacggcaacgaccccacagatctgattaagcacttccgaaagtatcacttaggactgcataaccgcaccaggcaagatgctgagctggacagcaaaatcttggcccttcataacatggtgcagttcagccattccaaagacttccagaaggtcaaccgttctgtgttttctggtgtgctgcaggacatcaattcttcaaggcctgttttactaaatgggacctatgatgtgcaggtgacttcaggtggaacattcattggcattggacggaaaacaccagattgccaagggaacaccaagtatttccgctgtaaattctgcaatttcacttatatgggcaactcatccaccgaattagaacaacattttcttcagactcacccaaacaaaataaaagcttctctcccctcctctgaggttgcaaaaccttcagagaaaaactctaacaagtccatccctgcacttcaatccagtgattctggagacttgggaaaatggcaggacaagataacagtcaaagcaggagatgacactcctgttgggtactcagtgcccataaagcccctcgattcctctagacaaaatggtacagaggccaccagttactactggtgtaaattttgtagtttcagctgtgagtcatctagctcacttaaactgctagaacattatggcaagcagcacggagcagtgcagtcaggcggccttaatccagagttaaatgataagctttccaggggctctgtcattaatcagaatgatctagccaaaagttcagaaggagagacaatgaccaagacagacaagagctcgagtggggctaaaaagaaggacttctccagcaagggagccgaggataatatggtaacgagctataattgtcagttctgtgacttccgatattccaaaagccatggccctgatgtaattgtagtggggccacttctccgtcattatcaacagctccataacattcacaagtgtaccattaaacactgtccattctgtcccagaggactttgcagcccagaaaagcaccttggagaaattacttatccgtttgcttgtagaaaaagtaattgttcccactgtgcactcttgcttctgcacttgtctcctggggcggctggaagctcgcgagtcaaacatcagtgccatcagtgttcattcaccacccctgacgtagatgtactcctctttcactatgaaagtgtgcatgagtcccaagcatcggatgtcaaacaagaagcaaatcacctgcaaggatcggatgggcagcagtctgtcaaggaaagcaaagaacactcatgtaccaaatgtgattttattacccaagtggaagaagagatttcccgacactacaggagagcacacagctgctacaaatgccgtcagtgcagttttacagctgccgatactcagtcactactggagcacttcaacactgttcactgccaggaacaggacatcactacagccaacggcgaagaggacggtcatgccatatccaccatcaaagaggagcccaaaattgacttcagggtctacaatctgctaactccagactctaaaatgggagagccagtttctgagagtgtggtgaagagagagaagctggaagagaaggacgggctcaaagagaaagtttggaccgagagttccagtgatgaccttcgcaatgtgacttggagaggggcagacatcctgcgggggagtccgtcatacacccaagcaagcctggggctgctgacgcctgtgtctggcacccaagagcagacaaagactctaagggatagtcccaatgtggaggccgcccatctggcgcgacctatttatggcttggctgtggaaaccaagggattcctgcagggggcgccagctggcggagagaagtctggggccctcccccagcagtatcctgcatcgggagaaaacaagtccaaggatgaatcccagtccctgttacggaggcgtagaggctccggtgttttttgtgccaattgcctgaccacaaagacctctctctggcgaaagaatgcaaatggcggatatgtatgcaacgcgtgtggcctctaccagaagcttcactcgactcccaggcctttaaacatcattaaacaaaacaacggtgagcagattattaggaggagaacaagaaagcgccttaacccagaggcacttcaggctgagcagctcaacaaacagcagaggggcagcaatgaggagcaagtcaatggaagcccgttagagaggaggtcagaagatcatctaactgaaagtcaccagagagaaattccactccccagcctaagtaaatacgaagcccagggttcattgactaaaagccattctgctcagcagccagtcctggtcagccaaactctggatattcacaaaaggatgcaacctttgcacattcagataaaaagtcctcaggaaagtactggagatccaggaaatagttcatccgtatctgaagggaaaggaagttctgagagaggcagtcctatagaaaagtacatgagacctgcgaaacacccaaattattcaccaccaggcagccctattgaaaagtaccagtacccactttttggacttccctttgtacataatgacttccagagtgaagctgattggctgcggttctggagtaaatataagctctccgttcctgggaatccgcactacttgagtcacgtgcctggcctaccaaatccttgccaaaactatgtgccttatcccaccttcaatctgcctcctcatttttcagctgttggatcagacaatgacattcctctagatttggcgatcaagcattccagacctgggccaactgcaaacggtgcctccaaggagaaaacgaaggcaccaccaaatgtaaaaaatgaaggtcccttgaatgtagtaaaaacagagaaagttgatagaagtactcaagatgaactttcaacaaaatgtgtgcactgtggcattgtctttctggatgaagtgatgtatgctttgcatatgagttgccatggtgacagtggacctttccagtgcagcatatgccagcatctttgcacggacaaatatgacttcacaacacatatccagaggggcctgcataggaacaatgcacaagtggaaaaaaatggaaaacctaaagagtaaaaccttagcacttagcacaattaaatagaaataggttttcttgatgggaattcaatagcttgtaatgtcttatgaagacctattaaaaaaatacttcatagagcctgccttatccaacatgaaattcccttcttttgttattctttcttttgatgagtaggttaccaagattaaaaagtgagataaatggtcaatgagaaagaatggaagatggtaaacaatcactttttaaaacctgttaagtcaaaaccatcttggctaatatgtactggggaaataatccataagagatatcaccagactagaattaatatatttataaagaaagagaccaaaactgtctagaatttgaaagggtttacatattattatactaaagcagtactggactggccattggaccatttgttccaaaacccataaattgttgcctaaatttataatgatcatgaaaccctaggcagaggaggagaaattgaaggtccagggcaatgaaagaaaaatggcgccctctcaatttagtcttctctcattggccatgtttcagattttgacctagaaatgcgagctgtggttaggcttggttagagtgcagcaagcaacatgacagatggtggcacgctgtttttacccagccctgcctgtacatacacatgcacaccctctctgatatttttgtcctttagatgttcaaatactcagtagtccttttgtttgcggtttagattcattttgtccacacatgtacccattttaaaaaacaatgtcctcgatgcttctgtagtgatttcattttagccaggtatttctttcttgtgtgtgatgaaccagtatggatttgcttttctaagcctcctgttggttactaatctcacttggcacattataactaaaggaatcccctcaattcaaaagcatagatggatacaaatgtcagaccgtgggtttaatttgtttagaacacatggcatttcttcacaaggtaacctgctgtatttatttattttcttttggttaaatataatttccaaactttgtggtcaggcagcgtctaaggttacgttaccacagactgacagttggtatatgtaccagccaatcccttcattaaatgtatacagatttagttaagtagcattaaataggattcttagaagtatgtcctcatagaacttttaatacttaaggctttgtaaaaactatccatgaagggaaagctcctcagcataactgctcagggaaatagggctaaataactgaacattaaataattggttaaaggtgctgttagtcgagcctcaatgcttgctacaaggatgtatgtacaaggactgactttaataatttgcattatattgtcccaaccagtagtttattttttgccacggagatgtagaagatattacaagctactggatgcactgtcagattaacttatttcattaaagaagttgggagaacaaataggaaaaaaaaaacttatttttctagtaaatattaatgtattacatttcaaataatggtgcctgacatattgaataattattttctacagtgtacgtatgcaacaaagatattccatcatgcattagagtcagttctggctctgcctagctgtttacatttgcaaatgtagcaaacaaggtaatgaagcaactatttctattgcagtagatatccttttgtgtgtgtgtgtgtgcattaaagttgtaaacggtaacatgaaacaaatgaaagttcttgctataatggtatggaaaacaagaaggaaatgaaaatatttttatgcctacttaggaaaaaaagggtagcacttattcattccaagtacttttttttttttaatttttaagctcttaactcacattgttatgcttaagatgataaacatatatcctctttttattgctttgtctatgtttcatatgaaacatttcagaaattattttgataagtgttgctggaatctgcaacgctgatttttttttgcattctgtagtcgcatttgcactccatttttacattaattcgcagttgctttgtatcattgttttgtttgggttttgtttctttttcacagtgccgggtcttcgtttcttaaagttggatggcaggtagagttcaaccagttcgtgactgttgtagcgaatgaagttaaaaaaatgtctttctgatgttgtgttgtcattttcatttttgcatttttttgtttgcatattaaaaaaagagaaaagagaaagcaagagacagaaatcaggactaagtcctctgcttcagtttcattgttaacgggccttattctgatctcacctgtcgcgtagctctaatattcacataaactgaaataaagaagtggaatgaggagctttgacattcaaattatgtgatgtaatttatcttccttaggaattttgatggatgcatctcaaaatgtatagccagacttgagaggtgacaattaaagatctaaaaaagagaggagattcccccaaacaacaatatttaattttcttagtaaaaagaataacagaatgcatcgtggcaatccttaagcaacattatctatgtggactgcttaaatcagcaaaacaccagaagtttggttaacttgggcaatatgacaagtattactttttgggcaaaactactcattaagcaatttctctagtgtgtcggacacaaataggttctttatttttggcatgtatgcctttttattttcattcaattttttttttttctcagacagacatagtagtatcaactagcattggaaaatacatatcactattcttggaatatttatggtcagtctactttttagtaaaatatttttggatagcgttgacacgatagatcttattccatacttctttattattgataattttattttcattttttgctttcattattatacatattttggtggagaagaggttgggcttttttgaaagagacaaaaatttattataacactaaacactccttttttgacatattaaagcctttattccatctctcaagatatattataaaatttatttttttaatttaagatttctgaattattttatcttaaattgtgattttaaacgagctattatggtacggaactttttttaatgaggaatttcatgatgatttaggaattttctctcttggaaaaggcttcccctgtgatgaaaatgatgtgccagctaaaattgtgtgccatttaaaaactgaaaatattttaaaattatttgtctatattctaaattgagctttggatcaaactttaggccaggaccagctcatgcgttctcattcttccttttctcactctttctctcatcactcacctctgtattcattctgttgtttgggatagaaaaatcataaagagccaacccatctcagaacgttgtggattgagagagacactacatgactccaagtatatgagaaaaggacagagctctaattgataactctgtagttcaaaaggaaaagagtatgcccaattctctctacatgacatattgagattttttttaatcaacttttaagatagtgatgttctgttctaaactgttctgttttagtgaaggtagatttttataaaacaagcatggggattcttttctaaggtaatattaatgagaagggaaaaaagtatctttaacagctctttgttgaagcctgtggtagcacattatgtttataattgcacatgtgcacataatctattatgatccaatgcaaatacagctccaaaaatattaaatgtatatatattttaaaatgcctgaggaaatacatttttcttaataaactgaagagtctcagtatggctattaaaataattattagcctcctgttgtgtggctgcaaaacatcacaaagtgaccggtcttgagacctgtgaactgctgccctgtttagtaaataaaattaatgcatttctagagggggaatatctgccatccagtggtggaaatgtggagtaaagaagctggtggtctgcttctgtgctgtatgccagccttttgccttaagttgagaggaggtcaactttagctactgtctttggtttgagagccatggcaaaaaaaaaaaaagaaaaaaagatcaagtcgtctttggtgagccagtaaggtgaaagcttgctgactgtccaaggcacaagagaaaattgaggaattgaaatgcaacctgagtatcaaactaaatattctaatcaaaggtaggtactgttaggtggaattctatcagcaggcaactgcaaatgagaagaagatagaaggacgcccgtcgggactttggagggcattgttattttcccaaagaaagacggccaagggcagaggcatggattctttgcagagcacttccttttggtttttcagtactgtttcatagacagtgggctcacatgttcctgatagtgctgcagttgcttagaaagcatcccagttaattgcagtaattagaacttctggaatatgctagggcagaagtatgtcaagtatgtcacatgaagaaaatgtgaaattcaagagtaatccacacgtgagaaactagacaatgtacattcatgtgttctcttgaaaggaaagggagagctgtaagcttcactctgtcctacaccggagaaaagcaggaataactttaccgtggaaataatgtttagcttttatcagagaaaattgtccttctagagcatagagtcccaaaactcaattctggttttcccctgttttttttttttttttttttcccaacatatgaactgcagcatatcactttttctttttgtgcctcaggttcctcacctgtaaaattgaaaaatatatgtattaataatattattaataataataatggtaatgtagtacttgtttgtaaagcactttgagatccttggttgaaaggcaccataggagtgccaagtattattatgtggccaagggggttatttaaactgtcagttcccaaaggccaggaaaggttggggtcatttttcttaaagacgagctgtaaatatcaactaggcagccaatagtgttgactatgaagatgcaaaactattactaggctgataaaatcatagtttcttaatggctaccaataaggcaaatatcacaataataaacgccaaattccttagggcggactatttgacaaccacatggaaaactttgggggaggcatgaggggggaacatctcaaaatgccaatgtaaaatttaacttacagcaatattcaccagcagaaaatgtctttcatatggaatgatttcatgttgctaagaaaaagaattcaatttgtagtcctgatttgaatactagaatgttggctataatagttctgttcttacaacacatgaaattttttcgttttattttattttgttttcatagtgcatgttcatttctactcacaaacatgttcttggtgtatttcttatgcaaacaatcttcaggcagcaaagatgtctgttacatctaaacttgaataataaagttttaccaccagttacaca

SEQ ID NO: 68 - protein, 1294 aa, Homo sapiens
MPYEVNAGYDFTNMVRKKNPPLRNVASEGEGQILEPIGTESKVSGKNKEFSADQMSENTDQSDAAELNHKEEHSLHVQDPSSSSKKDLKSAVLSEKAGFNYESPSKGGNFPSFPHDEVTDRNMLAFSFPAAGGVCEPLKSPQRAEADDPQDMACTPSGDSLETKEDQKMSPKATEETGQAQSGQANCQGLSPVSVASKNPQVPSDGGVRLNKSKTDLLVNDNPDPAPLSPELQDFKCNICGYGYYGNDPTDLIKHFRKYHLGLHNRTRQDAELDSKILALHNMVQFSHSKDFQKVNRSVFSGVLQDINSSRPVLLNGTYDVQVTSGGTFIGIGRKTPDCQGNTKYFRCKFCNFTYMGNSSTELEQHFLQTHPNKIKASLPSSEVAKPSEKNSNKSIPALQSSDSGDLGKWQDKITVKAGDDTPVGYSVPIKPLDSSRQNGTEATSYYWCKFCSFSCESSSSLKLLEHYGKQHGAVQSGGLNPELNDKLSRGSVINQNDLAKSSEGETMTKTDKSSSGAKKKDFSSKGAEDNMVTSYNCQFCDFRYSKSHGPDVIVVGPLLRHYQQLHNIHKCTIKHCPFCPRGLCSPEKHLGEITYPFACRKSNCSHCALLLLHLSPGAAGSSRVKHQCHQCSFTTPDVDVLLFHYESVHESQASDVKQEANHLQGSDGQQSVKESKEHSCTKCDFITQVEEEISRHYRRAHSCYKCRQCSFTAADTQSLLEHFNTVHCQEQDITTANGEEDGHAISTIKEEPKIDFRVYNLLTPDSKMGEPVSESVVKREKLEEKDGLKEKVWTESSSDDLRNVTWRGADILRGSPSYTQASLGLLTPVSGTQEQTKTLRDSPNVEAAHLARPIYGLAVETKGFLQGAPAGGEKSGALPQQYPASGENKSKDESQSLLRRRRGSGVFCANCLTTKTSLWRKNANGGYVCNACGLYQKLHSTPRPLNIIKQNNGEQIIRRRTRKRLNPEALQAEQLNKQQRGSNEEQVNGSPLERRSEDHLTESHQREIPLPSLSKYEAQGSLTKSHSAQQPVLVSQTLDIHKRMQPLHIQIKSPQESTGDPGNSSSVSEGKGSSERGSPIEKYMRPAKHPNYSPPGSPIEKYQYPLFGLPFVHNDFQSEADWLRFWSKYKLSVPGNPHYLSHVPGLPNPCQNYVPYPTFNLPPHFSAVGSDNDIPLDLAIKHSRPGPTANGASKEKTKAPPNVKNEGPLNVVKTEKVDRSTQDELSTKCVHCGIVFLDEVMYALHMSCHGDSGPFQCSICQHLCTDKYDFTTHIQRGLHRNNAQVEKNGKPKE

SEQ ID NO: 69 - DNA, 387 bp, Homo sapiens
atggggtttggagaccagggaacggtggaagggagcctaggaacgtcgaaaaaaccacctgaagtgaaaatgtttggagccagtcaaggtttgctgacaatggaaacaaaccagtccctggcacaaggcacaggctgctcagtagtaaaggtagacactgttctcttcgagagtttataccactgcggctttgaacacgggagcgtgatgcactgccttggggatgatcacccccaggaagacaggaaggctcacttctctgccccagttgcagccatcgcctctccagcaccgactcctgtctgtcctgcacaccactcaacacagagcatctgccagtttctacagcactgcaggcagaacactcacttgcaggctgctaactaa

SEQ ID NO: 70 - protein, 128 aa, Homo sapiens
MGFGDQGTVEGSLGTSKKPPEVKMFGASQGLLTMETNQSLAQGTGCSVVKVDTVLFESLYHCGFEHGSVMHCLGDDHPQEDRKAHFSAPVAAIASPAPTPVCPAHHSTQSICQFLQHCRQNTHLQAAN

SEQ ID NO: 71 - DNA, 5393 bp, Homo sapiens
ggggaacagcaaattcagtcacagacaatcctccactcggtcaagagccacttttctcttcctgccttgcccccccgcagggggtaaggaactgagcgtttaatctttagccggttggctaccagctaaaattctacttatcttagtttctagtggatagctttcttattttgcccatgttttcttagaatccctgtttaatatacttttgtcagtagtagtatctaggagtagcagggagagtgacaataaattagccccttcttttttcccttgtcattcaggccccttttcctctccagagggaaattaccagtaaactcttctaaatcttccaccccttctcagtcatactgtgaagaaacacactaaagtggacattatttgaccagtgaacacgaacccagcttcaggcattggtttgttgtggcacatggagaaacatctcttttaaaatatctcccaattacccttttcacaatttgtatccacctaggatttgctgctggggtaagtcactagatttatttctcaaagctcccctctctatgagctgaaagactgaccaaccatgaacactagtaggggatggggaaaggggacagagcagagccagttgttccacactttgggaagcaggagtagcttttatcatcttcctctggggagcaggcatagagacataaactgagtgaaaatgggtggaggaagaacttctatacccacgaacaacatgtgaagagagagaaccaaacataaagtaaggagggtagacgttacatccaagaggaaataatccaggcaaggaagcacaagctgatcaagatgtgtagttctgtggctgccaagttgtggtttttgacagatcgtcgcatcagggaagactatcctcaaaaagagattttacgagcattgaaggccaaatgttgtgaggaggaactggactttagggctgtggtgatggatgaggtggtgctgacaatcgagcaaggaaacctgggtctgcggatcaatggagagctaatcactgcctacccacaagtggtggtagtcagagtaccaaccccttgggtgcaaagtgatagtgacatcactgttttgcgccatctagagaagatgggatgtcggttaatgaaccgacctcaagccatcctgaactgcgttaataagttctggacatttcaagagttggctggccatggtgttcctctgccggatactttctcttatggtggccacgaaaattttgctaaaatgattgatgaggctgaagttctggagttcccaatggtagtaaagaatacgcggggtcacagaggtaaagctgttttcttggctcgagataagcaccatttggctgatctaagccatcttattcgccatgaagcgccatacctgttccagaagtatgttaaagagtctcatggacgggatgtacgtgtcattgtcgtgggaggccgtgtggttggcaccatgttacgttgttcaacagatgggagaatgcaaagcaactgctcattaggtggtgtggggatgatgtgctcattgagtgaacaagggaagcagctagctatccaggtgtctaatatcctggggatggatgtgtgtggcattgacctgctgatgaaagatgacggctccttctgcgtctgtgaggccaatgcaaatgtaggtttcatcgcctttgataaggcttgtaatctagatgtagctggtatcatagcagactatgccgcctcccttctaccctctggccggctcacccggcgtatgtccctgctctccgtggtgtccactgccagtgagactagtgagccggagctgggtcccccagccagcactgctgttgacaacatgagtgcaagttccagctctgttgacagcgaccctgaaagcacggagcgagagctgctcaccaagctcccagggggcctgttcaacatgaaccagctgctagccaatgaaatcaaactactggtggactgactccactggtaattaaccaacaaaacccttgtaaaactttctttcttcttttctatttttaaaaccaacttgcaatgctgttcatggaggatgctcaggaagatgagagaaaattagtaggattagttggagagagtgggagatagatgagacctctgctagtaagatgttactttcatttacaaatcctacaaatagagaggcagaataggtggggtatagaaaaatgtcaggctctcatagttacccttttaaattgctaaaaaatgtgtatgctcataggccatgaggaacaaatactttttttttttcatggtcccttgcttttgtttttgtacaaaaaaaaatggttttgctacaaatatccaagtagcataacttcacattgtgttggaagatttgtcatcagtgaggaaaacatctgcttaaattacaggaatttttgtattatacagctctgaaaattctgccatttccttattaactagcagctttagtttgtagtttatgaaatcttgaggggctcttttactgggatttcttatttttttgttttttcccgcttaatttggtgggaggtcaaattgaatataacccaataaaggcttcttaatgacaaaattggcatgtttgcatgatgaaatggaaatgaacagtattgcaatgtccggtatacaaaataacattaattcaatgtagataaaattacactagtttaaaatatgtgcattcacttgtatttgttagtgttttagtcttttttgaaagatgtgctctgttaatgttgctttttttttttttttttaatacatgctagtctaacatttcctgctctatgcctgcatctttaacaatggccaaagtgaagaaaatgctaccttttttgttaacaagacactgacttgaaacatgtacatttaaagccttttattttttccctttttgttttggtagttgggcatttaaataaggacaaggaaaaatatttttgggggcaaatcaagagcctatgagttctaagtataaagctgaagtgatttcgaatgccagcgttatatatttgcatttttcacattttacgagggagtatatgtgtatgtgtgtgcacgcatgcatgtgtatgtgttttgctttttgtttccatcaactaatcaaaaaggataatttagaaaatggagcatgatgggaaacagagtttttgactttaaaaaacagatgagttgttttcataagtagactccactggggtagaggtattcaccttaaaacatagggtgagtagatgcttttttaggcctttttgtgtatatgtacgttgtttgtttttttccttttgtttctagcctgttcagtgtacagtttattcaaggctacatgcttttctttaatgcttctggctatgcattttctctttttacatataggatttgggattgggggtgggttggatgtttttgtttggggacttatttagtagtattgagtctcttatagccctactcttaagccttcaatactgtccactctttatattcctttacttgcagaatttataaaagcccccaaactgcatataatatgagcctttaaaacatgggtaaaactaatcccattgatgggtttggatggtatgttaagaaatggagatgctgcagagcccaacgtaattttttaaacagcaagttttccatctccctacgaatcctctgaagcttttacccaagccctttcttgcctctccagtgctattttccttcagatggaccttaaacataatttcttggacactactagagagacttcgaggcaataataaaagatcagtattaaccagctataacagaggtttgatcatgcttacttgtacagtttttcccccgttttaaaaaggaatgtaataaaatttgttttttccatagaattaaataatattaaaattgagtgaaaggttgattgttgatgaatagaatagtacctctcatctgtgcagtgtctcatttcacctcagagaaaaggatacataagaggagtttgtaatttatcttaggatattctaattgcatttaaaagaacttatcttgcgcagggtaaatgggggactcacatacatatattaatacctctgactcattaacagaaagaaatacttggtacttctttcgctgaatgaccatactgtggaggatgcatactatttggtatagagaaataaatgaggaagaaagaactgcttaattaaattatcattcatatgttcatatagagaccatctggttgccatgtgtattatgacacatacactttgaatagttacatatcacaagtatgtagttcatgtttgtgttggtggggtaaggcatcaggaaaaatgtagttagtcttttcttaacttataccaaattaaccaactatattataggaaatatgtgaaattagttcattagctttattcactattatgcattcacatgatattaaaacgtacactcacatgttagaatgaaaagagcagtagttatcttagattttaaaaacatggatatcttcttgaattccttcaagattgaggtagagaataagagcaaatcattctggaagtaccttaaggaaacaaacagcagcagatatttaggttaaacttattttcataattgtttaataacttttgtataatcttcattgctattatgagagagaatgtatatatcaaatatgtgtaatgataaaatctgaattgtaaaatttttgtatattgttaaaattgtaattctaaattgtatttcaaaaatgattatttctgatattgtttttatgtcacccatgatgaaaactggactttatatatctaaacatacaagtatgaactattctatttaaaatttttaatagtttttttcttttttggtgcctataattgattggtcatttctgctggcttttctccaatgaacattgaaatcttcctgtatatgttaccaataagaaaactaccctggaacagtagaaaaacccaacaagagacttggcattcatcaagcacattatcagactttgagaacatattgaaggcattgactttgaaaatcatctctttttctcaagaagaaagcaatggagaagcaaatttgtttcattcagtgaatccccagtttggggcttgtggggcttagagacattgtgaaatcaaatcttgtgttatacttttctcctggctcactttttttgagaaggtttatgggctatttggctggtgagacacgatcccctcctaagaaaatgtaggtgctcagacaggtaaccactgctgctactgtttttatttgtttgtttgttcaattttatttaagatttgtttttgttgtactaggattttaaaaaatgtaatatattgcaggatttataaccag

SEQ ID NO: 72 - protein, 386 aa, Homo sapiens
MCSSVAAKLWFLTDRRIREDYPQKEILRALKAKCCEEELDFRAVVMDEVVLTIEQGNLGLRINGELITAYPQVVVVRVPTPWVQSDSDITVLRHLEKMGCRLMNRPQAILNCVNKFWTFQELAGHGVPLPDTFSYGGHENFAKMIDEAEVLEFPMVVKNTRGHRGKAVFLARDKHHLADLSHLIRHEAPYLFQKYVKESHGRDVRVIVVGGRVVGTMLRCSTDGRMQSNCSLGGVGMMCSLSEQGKQLAIQVSNILGMDVCGIDLLMKDDGSFCVCEANANVGFIAFDKACNLDVAGIIADYAASLLPSGRLTRRMSLLSVVSTASETSEPELGPPASTAVDNMSASSSSVDSDPESTERELLTKLPGGLFNMNQLLANEIKLLVD

SEQ ID NO: 73 - DNA, 1431 bp, Homo sapiens
ctgagtgggggcggggactgctggagttgcggggcctgcctggggtagggcggggcaggacagcttggagatagggcccggaattgcgggcgtcactctgctcctgcgacctagccaggcgtgagggagtgacagcagcgcattcgcgggacgagagcgatgagtgagaacgccgcaccaggtctgatctcagagctgaagctggctgtgccctggggccacatcgcagccaaagcctggggctccctgcagggccctccagttctctgcctgcacggctggctggacaatgccagctccttcgacagactcatccctcttctcccgcaagacttttattacgttgccatggatttcggaggtcatgggctctcgtcccattacagcccaggtgtcccatattacctccagacttttgtgagtgagatccgaagagttgtggcagccttgaaatggaatcgattctccattctgggccacagcttcggtggcgtcgtgggcggaatgtttttctgtaccttccccgagatggtggataaacttatcttgctggacacgccgctctttctcctggaatcagatgaaatggagaacttgctgacctacaagcggagagccatagagcacgtgctgcaggtagaggcctcccaggagccctcgcacgtgttcagcctgaagcagctgctgcagaggttactgaagagcaatagccacttgagtgaggagtgcggggagcttctcctgcaaagaggaaccacgaaggtggccacaggtctggttctgaacagagaccagaggctcgcctgggcagagaacagcattgacttcatcagcagggagctgtgtgcgcattccatcaggaagctgcaggcccatgtcctgttgatcaaagcagtccacggatattttgattcaagacagaattactctgagaaggagtccctgtcgttcatgatagacacgatgaaatccaccctcaaagagcagttccagtttgtggaagtcccaggcaatcactgtgtccacatgagcgaaccccagcacgtggccagtatcatcagctccttcttacagtgcacacacatgctcccagcccagctgtagctctgggcctggaactatgaagacctagtgctcccagactcaacactgggactctgagttcctgagccccacaacaaggccagggatggtggggacaggcctcactagtcttgaggcccagcctaggatggtagtcaggggaaggagcgagattccaacttcaacatctgtgacctcaagggggagacagagtctgggttccagggctgctttctcctggctaataataaatatccagccagctggaggaaggaagggcaggctgggcccacctagcctttccctgctgcccaactggatggaaaataaaaggttcttgtattctca

SEQ ID NO: 74 - protein, 314 aa, Homo sapiens
MSENAAPGLISELKLAVPWGHIAAKAWGSLQGPPVLCLHGWLDNASSFDRLIPLLPQDFYYVAMDFGGHGLSSHYSPGVPYYLQTFVSEIRRVVAALKWNRFSILGHSFGGVVGGMFFCTFPEMVDKLILLDTPLFLLESDEMENLLTYKRRAIEHVLQVEASQEPSHVFSLKQLLQRLLKSNSHLSEECGELLLQRGTTKVATGLVLNRDQRLAWAENSIDFISRELCAHSIRKLQAHVLLIKAVHGYFDSRQNYSEKESLSFMIDTMKSTLKEQFQFVEVPGNHCVHMSEPQHVASIISSFLQCTHMLPAQL

SEQ ID NO: 75 - DNA, 1442 bp, Homo sapiens
ggttcccttccacgctgtgaagctttgttcttttggtcttcatgataaatcttgctgctgctcactcgttgggtccgtgccacctttaagagctgtaacactcaccgcgaaggtctgcaacttcactcctggggccagcaagaccacgaatgcaccgagaggaatgaacaactctggacacaccatctttaagaaccgtaatactcaccgcaagggtctgcaacttcattcttgaagtcagtgaggccaagaacccatcaattccgtacacattttggtgactttgaagagactgtcacctatcaccaagtggtgagactattgccaagcagtgagactattgccaagtggtgagaccatcaccaagcggtgagactatcacctatcgccaagtggcctgattcagcaggaagcatctcagacaccaaccactatgctgtcagcagttgcccggggctaccagggctggtttcatccctgtgctaggctttctgtgaggatgagcagcaccgggatagacaggaagggcgtcctggctaaccgggtagccgtggtcacggggtccaccagtgggatcggctttgccatcgcccgacgtctggcccgggacggggcccacgtggtcatcagcagccggaagcagcagaacgtggaccgggccatggccaagctgcagggggaggggctgagtgtggcgggcattgtgtgccacgtggggaaggctgaggaccgggagcagctggtggccaaggccctggagcactgtgggggcgtcgacttcctggtgtgcagcgcaggggtcaaccctctggtagggagcactctggggaccagtgagcagatctgggacaagatcctaagtgtgaacgtgaagtccccagccctgctgctgagccagttgctgccctacatggagaacaggaggggtgctgtcatcctggtctcttccattgcagcttataatccagtagtggcgctgggtgtctacaatgtcagcaagacagcgctgctgggtctcactagaacactggcattggagctggcccccaaggacatccgggtaaactgcgtggttccaggaattataaaaactgacttcagcaaagtgtttcatgggaatgagtctctctggaagaacttcaaggaacatcatcagctgcagaggattggggagtcagaggactgtgcaggaatcgtgtccttcctgtgctctccagatgccagctacgtcaacggggagaacattgcggtggcaggctactccactcggctctgagaggagtgggggcggctgcgtagctgtggtcccagcccaggagcctgagggggtgtctaggtgatcatttggatctggagcagagtctgccattctgccagactagcaatttgggggcttactcatgctaggcttgaggaagaagaaaaacgcttcggcattctcc

SEQ ID NO: 76 - protein, 280 aa, Homo sapiens
MLSAVARGYQGWFHPCARLSVRMSSTGIDRKGVLANRVAVVTGSTSGIGFAIARRLARDGAHVVISSRKQQNVDRAMAKLQGEGLSVAGIVCHVGKAEDREQLVAKALEHCGGVDFLVCSAGVNPLVGSTLGTSEQIWDKILSVNVKSPALLLSQLLPYMENRRGAVILVSSIAAYNPVVALGVYNVSKTALLGLTRTLALELAPKDIRVNCVVPGIIKTDFSKVFHGNESLWKNFKEHHQLQRIGESEDCAGIVSFLCSPDASYVNGENIAVAGYSTRL

SEQ ID NO: 77 - DNA, 1743 bp, Homo sapiens
aaagaaggtaagggcagtgagaatgatgcatcttgcattccttgtgctgttgtgtctgccagtctgctctgcctatcctctgagtggggcagcaaaagaggaggactccaacaaggatcttgcccagcaatacctagaaaagtactacaacctcgaaaaggatgtgaaacagtttagaagaaaggacagtaatctcattgttaaaaaaatccaaggaatgcagaagttccttgggttggaggtgacagggaagctagacactgacactctggaggtgatgcgcaagcccaggtgtggagttcctgacgttggtcacttcagctcctttcctggcatgccgaagtggaggaaaacccaccttacatacaggattgtgaattatacaccagatttgccaagagatgctgttgattctgccattgagaaagctctgaaagtctgggaagaggtgactccactcacattctccaggctgtatgaaggagaggctgatataatgatctctttcgcagttaaagaacatggagacttttactcttttgatggcccaggacacagtttggctcatgcctacccacctggacctgggctttatggagatattcactttgatgatgatgaaaaatggacagaagatgcatcaggcaccaatttattcctcgttgctgctcatgaacttggccactccctggggctctttcactcagccaacactgaagctttgatgtacccactctacaactcattcacagagctcgcccagttccgcctttcgcaagatgatgtgaatggcattcagtctctctacggacctccccctgcctctactgaggaacccctggtgcccacaaaatctgttccttcgggatctgagatgccagccaagtgtgatcctgctttgtccttcgatgccatcagcactctgaggggagaatatctgttctttaaagacagatatttttggcgaagatcccactggaaccctgaacctgaatttcatttgatttctgcattttggccctctcttccatcatatttggatgctgcatatgaagttaacagcagggacaccgtttttatttttaaaggaaatgagttctgggccatcagaggaaatgaggtacaagcaggttatccaagaggcatccataccctgggttttcctccaaccataaggaaaattgatgcagctgtttctgacaaggaaaagaagaaaacatacttctttgcagcggacaaatactggagatttgatgaaaatagccagtccatggagcaaggcttccctagactaatagctgatgactttccaggagttgagcctaaggttgatgctgtattacaggcatttggatttttctacttcttcagtggatcatcacagtttgagtttgaccccaatgccaggatggtgacacacatattaaagagtaacagctggttacattgctaggcgagatagggggaagacagatatgggtgtttttaataaatctaataattattcatctaatgtattatgagccaaaatggttaatttttcctgcatgttctgtgactgaagaagatgagccttgcagatatctgcatgtgtcatgaagaatgtttctggaattcttcacttgcttttgaattgcactgaacagaattaagaaatactcatgtgcaataggtgagagaatgtattttcatagatgtgttattacttcctcaataaaaagttttattttgggcctgttcctt

SEQ ID NO: 78 - protein, 475 aa, Homo sapiens [sequence omitted]

SEQ ID NO: 79 - DNA, 3198 bp, Homo sapiens
atgctgccacaaataccctttttgctgctagtatccttgaacttggttcatggagtgttttacgctgaacgataccaaacgcccacaggcataaaaggcccactacccaacaccaagacacagttcttcattccctacaccataaagagtaaaggtatagcagtaagaggagagcaaggtactcctggtccaccaggccctgctggacctcgagggcacccaggtccttctggaccaccaggaaaaccaggctacggaagtcctggactccaaggagagccagggttgccaggaccaccgggaccatcagctgtagggaaaccaggtgtgccaggactcccaggaaaaccaggagagagaggaccatatggaccaaaaggagatgttggaccagctggcctaccaggaccccggggcccaccaggaccacctggaatccctggaccggctggaatttctgtgccaggaaaacctggacaacagggacccacaggagccccaggacccaggggctttcctggagaaaagggtgcaccaggagtccctggtatgaatggacagaaaggggaaatgggatatggtgctcctggtcgtccaggtgagaggggtcttccaggccctcagggtcccacaggaccatctggccctcctggagtgggaaaaagaggtgaaaatggggttccaggacagccaggcatcaaaggtgatagaggttttccgggagaaatgggaccaattggcccaccaggtccccaaggccctcctggggaacgagggccagaaggcattggaaagccaggagctgctggagccccaggccagccagggattccaggaacaaaaggtctccctggggctccaggaatagctgggcccccagggcctcctggctttgggaaaccaggcttgccaggcctgaagggagaaagaggacctgctggccttcctgggggtccaggtgccaaaggggaacaagggccagcaggtcttcctgggaagccaggtctgactggaccccctgggaatatgggaccccaaggaccaaaaggcatcccgggtagccatggtctcccaggccctaaaggtgagacagggccagctgggcctgcaggataccctggggctaagggtgaaaggggttcccctgggtcagatggaaaaccagggtacccaggaaaaccaggtctcgatggtcctaagggtaacccagggttaccaggtccaaaaggtgatcctggagttggaggacctcctggtctcccaggccctgtgggcccagcaggagcaaagggaatgcccggacacaatggagaggctggcccaagaggtgcccctggaataccaggtactagaggccctattgggccaccaggcattccaggattccctgggtctaaaggggatccaggaagtcccggtcctcctggcccagctggcatagcaactaagggcctcaatggacccaccgggccaccagggcctccaggtccaagaggcccctctggagagcctggtcttccagggccccctgggcctccaggcccaccaggtcaagcagtcatgcctgagggttttataaaggcaggccaaaggcccagtctttctgggacccctcttgttagtgccaaccagggggtaacaggaatgcctgtgtctgcttttactgttattctctccaaagcttacccagcaataggaactcccataccatttgataaaattttgtataacaggcaacagcattatgacccaaggactggaatctttacttgtcagataccaggaatatactatttttcataccacgtgcatgtgaaagggactcatgtttgggtaggcctgtataagaatggcacccctgtaatgtacacctatgatgaatacaccaaaggctacctggatcaggcttcagggagtgccatcatcgatctcacagaaaatgaccaggtgtggctccagcttcccaatgccgagtcaaatggcctatactcctctgagtatgtccactcctctttctcaggattcctagtggctccaatgtgagtacaccccacagagctaatctaaatcttgtgctagaaaaagcattctctaactctaccccaccctacaaaatgcatatggaggtaggctgaaaagaatgtaatttttattttctgaaatacagatttgagctatcagaccaacaaaccttccccctgaaaagtgagcagcaacgtaaaaacgtatgtgaagcctctcttgaatttctagttagcaatcttaaggctctttaaggttttctccaatattaaaaaatatcaccaaagaagtcctgctatgttaaaaacaaacaacaaaaaacaaagcaacaaaaaaaaaaattaaaaaaaaaaacagaaatagagctctaagttatgtgaaatttgatttgagaaactcggcatttcctttttaaaaaagcctgtttctaactatgaatatgagaacttctaggaaacatccaggaggtatcatataactttgtagaacttaaatacttgaatattcaaatttaaaagacactgtatcccctaaaatatttctgatggtgcactactctgaggcctgtatggcccctttcatcaatatctattcaaatatacaggtgcatatatacttgttaaagctcttatataaaaaagccccaaaatattgaagttcatctgaaatgcaaggtgctttcatcaatgaaccttttcaaaacttttctatgattgcagagaagctttttatatacccagcataacttggaaacaggtatctgacctattcttatttagttaacacaagtgtgattaatttgatttctttaattccttattgaatcttatgtgatatgattttctggatttacagaacattagcacatgtaccttgtgcctcccattcaagtgaagttataatttacactgagggtttcaaaattcgactagaagtggagatatattatttatttatgcactgtactgtatttttatattgctgtttaaaacttttaagctgtgcctcacttattaaagcacaaaatgttttacctactccttatttacgacacaataaaataacatcaatagatttttaggctgaattaatttgaaagcagcaatttgctgttctcaaccattctttcaaggcttttcattcgacacaataaaataacatcaatagatttttagg

SEQ ID NO: 80 - protein, 680 aa, Homo sapiens
MLPQIPFLLLVSLNLVHGVFYAERYQTPTGIKGPLPNTKTQFFIPYTIKSKGIAVRGEQGTPGPPGPAGPRGHPGPSGPPGKPGYGSPGLQGEPGLPGPPGPSAVGKPGVPGLPGKPGERGPYGPKGDVGPAGLPGPRGPPGPPGIPGPAGISVPGKPGQQGPTGAPGPRGFPGEKGAPGVPGMNGQKGEMGYGAPGRPGERGLPGPQGPTGPSGPPGVGKRGENGVPGQPGIKGDRGFPGEMGPIGPPGPQGPPGERGPEGIGKPGAAGAPGQPGIPGTKGLPGAPGIAGPPGPPGFGKPGLPGLKGERGPAGLPGGPGAKGEQGPAGLPGKPGLTGPPGNMGPQGPKGIPGSHGLPGPKGETGPAGPAGYPGAKGERGSPGSDGKPGYPGKPGLDGPKGNPGLPGPKGDPGVGGPPGLPGPVGPAGAKGMPGHNGEAGPRGAPGIPGTRGPIGPPGIPGFPGSKGDPGSPGPPGPAGIATKGLNGPTGPPGPPGPRGPSGEPGLPGPPGPPGPPGQAVMPEGFIKAGQRPSLSGTPLVSANQGVTGMPVSAFTVILSKAYPAIGTPIPFDKILYNRQQHYDPRTGIFTCQIPGIYYFSYHVHVKGTHVWVGLYKNGTPVMYTYDEYTKGYLDQASGSAIIDLTENDQVWLQLPNAESNGLYSSEYVHSSFSGFLVAPM

SEQ ID NO: 81 - DNA, 3381 bp, Homo sapiens
cgcgattctcagggattgatccgcctcttcaggtaagttatcttccggccccgtaccactgtgccacaggcgcagcccgcttcctcaggtgccctatcccgcgcagaagaccacggcttcacagagtgttatttaagggcgtggccagcggaacatcccgccccattctgtgacgcacggggtggcgcgcgtgggacccgaggggtggggctgggtttagtaggagacctggggcaaggccccctgtggacgaccatctgccagcttctctcgttccgtcgattgggaggagcggtggcgacctcggccttcagtgtttccgacggagtgaatggcggcggcggctgggatgctgctgctgggcttgctgcaggcgggtgggtcggtgctgggccaggcgatggagaaggtgacaggcggcaacctcttgtccatgctgctgatcgcctgcgccttcaccctcagcctggtctacctgatccgtctggccgccggccacctggtccagctgcccgcaggggtgaaaagtcctccatacattttctccccaattccattccttgggcatgccatagcatttgggaaaagtccaattgaatttctagaaaatgcatatgagaagtatggacctgtatttagttttaccatggtaggcaagacatttacttaccttctggggagtgatgctgctgcactgctttttaatagtaaaaatgaagacctgaatgcagaagatgtctacagtcgcctgacaacacctgtgtttgggaagggagttgcatacgatgtgcctaatccagttttcttggagcagaagaaaatgttaaaaagtggccttaacatagcccactttaaacagcatgtttctataattgaaaaagaaacaaaggaatactttgagagttggggagaaagtggagaaaaaaatgtgtttgaagctctttctgagctcataattttaacagctagccattgtttgcatggaaaggaaatcagaagtcaactcaatgaaaaggtagcacagctgtatgcagatttggatggaggtttcagccatgcagcctggctcttaccaggttggctgcctttgcctagtttcagacgcagggacagagctcatcgggaaatcaaggatattttctataaggcaatccagaaacgcagacagtctcaagaaaaaattgatgacattctccaaactttactagatgctacatacaaggatgggcgtcctttgactgatgatgaagtagcagggatgcttattggattactcttggcagggcagcatacatcctcaactactagtgcttggatgggcttctttttggccagagacaaaacacttcaaaaaaaatgttatttagaacagaaaacagtctgtggagagaatctgcctcctttaacttatgaccagctcaaggatctaaatttacttgatcgctgtataaaagaaacattaagacttagacctcctataatgatcatgatgagaatggccagaactcctcagactgtggcagggtataccattcctccaggacatcaggtgtgtgtttctcccactgtcaatcaaagacttaaagactcatgggtagaacgcctggactttaatcctgatcgctacttacaggataacccagcatcaggggaaaagtttgcctatgtgccatttggagctgggcgtcatcgttgtattggggaaaattttgcctatgttcaaattaagacaatttggtccactatgcttcgtttatatgaatttgatctcattgatggatactttcccactgtgaattatacaactatgattcacacccctgagaacccagttatccgttacaaacgaagatcaaaatgaaaaaggttgcaaggaacgaatatatgtgattatcactgtaagccacaaaggcattcgaagagaatgaagtgtacaaaacaactcttgtagtttactgtttttttaagtgtgtaattctaaaagccagtttatgatttaggattttgttaactgaatggttctatcaaatataatagcatttgacacattttctaatagttatgatacttatacatgtgctttcaggaagttccttggtgaaacaattgttgaggggggatctaggtaattggcagattctaaataatataatttccagatagtaattttaagagtactcatcgctcttgccaaataagttcagggtattcaaatcttggactagtcctgcaaggtataaagaataaaaatcccagtgagatacttggaaaccacagtttattattatttatctgggcaattattgtgtgtgtgaggatggaagggtagggaataatcgaacatctaaagccttgaataagagaatactaattgttttggtatgatgatactcagaaatggagatattataggaaaaagaaatcctttggaattttaactaaaatcactgcatatgggaaattaagagatccaggaccatatttgataagagttcctaaaaataatgtaattattaatgctaaagactgctcatgtatcttgatctaattactaaataaattacatatttatttacctgataaatatgtatctagttctacaaggtcacatttatgtggaagtccaaagtcaagtccttaggggataattttgttttgggctcagttgttccctgcttccttttttttttttttttttttttgagatggagtctcgctctgttgcccaggctggagtgcagtggtgcgatctcagctcactgcatcctctgcctcccgggttcaagcaattctctgcctcagcctcccaagtagttgggattacaggcacctgccaccatgcctggctaattttttgtatttttagtagagacgggggtttcactatgttggctaggctggtcttgaactcctgagcctcgtgagtccacccgccttggcctcccaaagtgctgggattacaggcatgagccaccgcacctggccttccctgcttcctctctagaatccaattagggatgtttgttactactcatattgattaaaacagttaacaaacttttttctttttaaaatgtgagatcagtgaactctggttttaagataatctgaaacaaggtccttgggagtaataaaattggtcacattctgtaaagcacattctgtttaggaatcaacttatctcaaattgtaactcggggcctaactatatgagatggctgaaaaaataccacatcgtctgttttcactaggtgatgccaaaatattttgctttatgtatattacagttctttttaaaacactggaagactcatgttaaactctaattgtgaaggcagaatctctgctaatttttcagattaaaattctctttgaaaaaat

SEQ ID NO: 82 - protein, 509 aa, Homo sapiens
MAAAAGMLLLGLLQAGGSVLGQAMEKVTGGNLLSMLLIACAFTLSLVYLIRLAAGHLVQLPAGVKSPPYIFSPIPFLGHAIAFGKSPIEFLENAYEKYGPVFSFTMVGKTFTYLLGSDAAALLFNSKNEDLNAEDVYSRLTTPVFGKGVAYDVPNPVFLEQKKMLKSGLNIAHFKQHVSIIEKETKEYFESWGESGEKNVFEALSELIILTASHCLHGKEIRSQLNEKVAQLYADLDGGFSHAAWLLPGWLPLPSFRRRDRAHREIKDIFYKAIQKRRQSQEKIDDILQTLLDATYKDGRPLTDDEVAGMLIGLLLAGQHTSSTTSAWMGFFLARDKTLQKKCYLEQKTVCGENLPPLTYDQLKDLNLLDRCIKETLRLRPPIMIMMRMARTPQTVAGYTIPPGHQVCVSPTVNQRLKDSWVERLDFNPDRYLQDNPASGEKFAYVPFGAGRHRCIGENFAYVQIKTIWSTMLRLYEFDLIDGYFPTVNYTTMIHTPENPVIRYKRRSK

SEQ ID NO: 83 - DNA, 444 bp, Homo sapiens
aattctagaagtccaaatcactcattgtttgtgaaagctgagctcacagcaaaacaagccaccatgaagctgtcggtgtgtctcctgctggtcacgctggccctctgctgctaccaggccaatgccgagttctgcccagctcttgtttctgagctgttagacttcttcttcattagtgaacctctgttcaagttaagtcttgccaaatttgatgcccctccggaagctgttgcagccaagttaggagtgaagagatgcacggatcagatgtcccttcagaaacgaagcctcattgcggaagtcctggtgaaaatattgaagaaatgtagtgtgtgacatgtaaaaactttcatcctggtttccactgtctttcaatgacaccctgatcttcactgcagaatgtaaaggtttcaacgtcttgctttaataaatcacttgctctac

SEQ ID NO: 84 - protein, 90 aa, Homo sapiens
MKLSVCLLLVTLALCCYQANAEFCPALVSELLDFFFISEPLFKLSLAKFDAPPEAVAAKLGVKRCTDQMSLQKRSLIAEVLVKILKKCSV

SEQ ID NO: 85 - DNA, 1780 bp, Homo sapiens
gggccaggactgagaaaggggaaagggaagggtgccacgtccgagcagccgccttgactggggaagggtctgaatcccacccttggcattgcttggtggagactgagatacccgtgctccgctcgcctccttggttgaagatttctccttccctcacgtgatttgagccccgtttttattttctgtgagccacgtcctcctcgagcggggtcaatctggcaaaaggagtgatgcgcttcgcctggaccgtgctcctgctcgggcctttgcagctctgcgcgctagtgcactgcgcccctcccgccgccggccaacagcagcccccgcgcgagccgccggcggctccgggcgcctggcgccagcagatccaatgggagaacaacgggcaggtgttcagcttgctgagcctgggctcacagtaccagcctcagcgccgccgggacccgggcgccgccgtccctggtgcagccaacgcctccgcccagcagccccgcactccgatcctgctgatccgcgacaaccgcaccgccgcggggcgaacgcggacggccggctcatctggagtcaccgctggccgccccaggcccaccgcccgtcactggttccaagctggctactcgacatctagagcccgcgaagctgggccctcgcgcgcggagaaccagacagcgccgggagaagttcctgctctcagtaacctgcggccgcccagccgcgtggacggcatggtgggcgacgacccttacaacccctacaagtactctgacgacaacccttattacaactactacgatacttatgaaaggcccagacctgggggcaggtaccggcccggatacggcactggctacttccagtacggtctcccagacctggtggccgacccctactacatccaggcgtccacgtacgtgcagaagatgtccatgtacaacctgagatgcgcggcggaggaaaactgtctggccagtacagcatacagggcagatgtcagagattatgatcacagggtgctgctcagatttccccaaagagtgaaaaaccaagggacatcagatttcttacccagccgaccaagatattcctgggaatggcacagttgtcatcaacattaccacagtatggatgagtttagccacttgtacctgcttgatgccaacacccagaggagatgggctgaaggccacaaagcaagtttctgtcttgaagacacatcctgtgactatggctaccacaggcgatttgcatgtactgcacacacacagggattgagtcctggctgttatgatacctatggtgcagacatagactgccagtggattgatattacagatgtaaaacctggaaactatatcctaaaggtcagtgtaaaccccagctacctggttcctgaatctgactataccaacaatgttgtgcgctgtgacattcgctacacaggacatcatgcgtatgcctcaggctgcacaatttcaccgtattagaaggcaaagcaaaactcccaatggataaatcagtgcctggtgttctgaagtgggaaaaaatagactaacttcagtaggatttatgtattttgaaaaagagaacagaaaacaacaaaagaatttttgtttggactgttttcaataacaaagcacataactggattttgaacgcttaagtcatcattacttgggaaatttttaatgtttattatttacatcactttgtgaattaacacagtgtttcaattctgtaattacatatttgactctttcaaaaaaaaaaaaaaaaaaaaaaa

SEQ ID NO: 86 - protein, 417 aa, Homo sapiens
MRFAWTVLLLGPLQLCALVHCAPPAAGQQQPPREPPAAPGAWRQQIQWENNGQVFSLLSLGSQYQPQRRRDPGAAVPGAANASAQQPRTPILLIRDNRTAAGRTRTAGSSGVTAGRPRPTARHWFQAGYSTSRAREAGPSRAENQTAPGEVPALSNLRPPSRVDGMVGDDPYNPYKYSDDNPYYNYYDTYERPRPGGRYRPGYGTGYFQYGLPDLVADPYYIQASTYVQKMSMYNLRCAAEENCLASTAYRADVRDYDHRVLLRFPQRVKNQGTSDFLPSRPRYSWEWHSCHQHYHSMDEFSHLYLLDANTQRRWAEGHKASFCLEDTSCDYGYHRRFACTAHTQGLSPGCYDTYGADIDCQWIDITDVKPGNYILKVSVNPSYLVPESDYTNNVVRCDIRYTGHHAYASGCTISPY

SEQ ID NO: 87 - DNA, 1216 bp, Homo sapiens
ttcggcacttgggagaagatgtttgaaaaaactgactctgctaatgagcctggactcagagctcaagtctgaactctacctccagacagaatgaagttcatctcgacatctctgcttctcatgctgctggtcagcagcctctctccagtccaaggtgttctggaggtctattacacaagcttgaggtgtagatgtgtccaagagagctcagtctttatccctagacgcttcattgatcgaattcaaatcttgccccgtgggaatggttgtccaagaaaagaaatcatagtctggaagaagaacaagtcaattgtgtgtgtggaccctcaagctgaatggatacaaagaatgatggaagtattgagaaaaagaagttcttcaactctaccagttccagtgtttaagagaaagattccctgatgctgatatttccactaagaacacctgcattcttcccttatccctgctctggattttagttttgtgcttagttaaatcttttccagggagaaagaacttccccatacaaataaggcatgaggactatgtgaaaaataaccttgcaggagctgatggggcaaactcaagcttcttcactcacagcaccctatatacacttggagtttgcattcttattcatcagggaggaaagtttctttgaaaatagttattcagttataagtaatacaggattattttgattatatacttgttgtttaatgtttaaaatttcttagaaaacaatggaatgagaatttaagcctcaaatttgaacatgtggcttgaattaagaagaaaattatggcatatattaaaagcaggcttctatgaaagactcaaaaagctgcctgggaggcagatggaacttgagcctgtcaagaggcaaaggaatccatgtagtagatatcctctgcttaaaaactcactacggaggagaattaagtcctacttttaaagaatttctttataaaatttactgtctaagattaatagcattcgaagatccccagacttcatagaatactcagggaaagcatttaaagggtgatgtacacatgtatcctttcacacatttgccttgacaaacttctttcactcacatctttttcactgactttttttgtgggggcggggccggggggactctggtatctaattctttaatgattcctataaatctaatgacattcaataaagttgagcaaacattttactt

SEQ ID NO: 88 - protein, 109 aa, Homo sapiens
MKFISTSLLLMLLVSSLSPVQGVLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 89 - DNA, 576 bp, Homo sapiens
cttctctgggacacattgccttctgttttctccagcatgcgcttgctccagctcctgttcagggccagccctgccaccctgctcctggttctctgcctgcagttgggggccaacaaagctcaggacaacactcggaagatcataataaagaattttgacattcccaagtcagtacgtccaaatgacgaagtcactgcagtgcttgcagttcaaacagaattgaaagaatgcatggtggttaaaacttacctcattagcagcatccctctacaaggtgcatttaactataagtatactgcctgcctatgtgacgacaatccaaaaaccttctactgggacttttacaccaacagaactgtgcaaattgcagccgtcgttgatgttattcgggaattaggcatctgccctgatgatgctgctgtaatccccatcaaaaacaaccggttttatactattgaaatcctaaaggtagaataatggaagccctgtctgtttgccacacccaggtgatttcctctaaagaaacttggctggaatttctgctgtggtctataaaataaacttcttaacatgctt

SEQ ID NO: 90 - protein, 145 aa, Homo sapiens
MRLLQLLFRASPATLLLVLCLQLGANKAQDNTRKIIIKNFDIPKSVRPNDEVTAVLAVQTELKECMVVKTYLISSIPLQGAFNYKYTACLCDDNPKTFYWDFYTNRTVQIAAVVDVIRELGICPDDAAVIPIKNNRFYTIEILKV

SEQ ID NO: 91 - DNA, 2340 bp, Homo sapiens
gatagcgccgggcagagggacccggctaccctggacagcgcatcgccgcccgcccgggtcgccgcgccacagccgctgcggatcatggaacatctaaaggcctttgatgatgaaatcaatgcttttttggacaatatgtttggaccgcgagattctcgagtcagagggtggttcacgttggactcttaccttcctaccttttttcttactgtcatgtatctgctctcaatatggctgggtaacaagtatatgaagaacagacctgctctttctctcaggggtatcctcaccttgtataatcttggaatcacacttctctccgcgtacatgctggcagagctcattctctccacttgggaaggaggctacaacttacagtgtcaagatcttaccagcgcaggggaagctgacatccgggtagccaaggtgctttggtggtactatttctccaaatcagtagagttcctggacacaattttcttcgttttgcggaaaaaaacgagtcagattacttttcttcatgtatatcatcatgcttctatgtttaacatctggtggtgtgtcttgaactggataccttgtggacaaagtttctttggaccaacactgaacagttttgtccacattcttatgtactcctactatggactttctgtgtttccatctatgcacaagtatctttggtggaagaaatatctcacacaggctcagctggtgcagttcgtgctcaccatcacgcacaccatgagcgccgtcgtgaaaccgtgtggcttccccttcggttgtctcatcttccagtcatcttatatgctaacgttagtcatcctcttcttaaatttttatgttcagacataccgaaaaaagccaatgaagaaagatatgcaagagccacctgcagggaaagaagtgaagaatggtttttccaaagcctacttcactgcagcaaatggagtgatgaacaagaaagcacaataaaaatgagtaacagaaaaagcacatatactagcctaacagattggcttgttttaaagcaaagactgaattgaaggttacatgttttaggataaactaatttcttttgagttcataaatcatttgtacccagaatgtattaatatattgctattaggttaatctgttaactgaatgctttgatcagcattgaggtgatgctcacctccgaggacctcagaactggtgcagcttctctctccctccctcccacagactgaacctttcgccagaagctgtccttataacgccttatacgcatacacagccaggaaacgtggagcattgtttctcacagagagtctccaaataaaaagggttttgttcagattaaaatgtttacaacaaaatgttaattatattctaaatacagggtatgttctaatctatattaagcaataatgccagtgcataatcattccatttgttcctttagcaatcaaccccagaaaatattaaaatgggatcatacacagaagatagaaaaatctagcaaaacttctctttctgtaagccagagtcttgtctatcagattcccacaaccactcctgattctaaatttagtgatatggtaatgaaattggtatttattttaaatattagttattctaaggagaaaaaaatgcttctgcaagattttcataattcaggggctgtggataggattgttcctctgtttccctaatcattcatctgttcatgtctccctcttgtgccagtcagcctaggttatacagatgccatgctccacaccacgagcagtgtacaaatctggctgcccgtttactttctgagcaagcactggagtccactccgacctttttctttgaacatgcatgctgctggaatatgtataaatcagaactagcagaagtagcagagtgatgggagcaaaataggcactgaattcgtcaactcttttttgtgagcctacttgtgaatattacctcagatacctgttgtcactcttcacaggttatttaagttcttgaagctgggaggaaaaagatggagtagcttggaaagattccagcactgagccgtgagccggtcatgagccacgataaaaaatgccagtttggcaaactcagcactcctgttccctgctcaggtatatgcgatctctactgagaagcaagcacaaaagtagaccaaagtattaatgagtatttcctttctccataagtgcaggactgttactcactactaaactctaccaagaatggaaaccaagaatattttctgaagatttttttgaagattaatttataccctataaaataaaacttgttagcttcgatgaagtcaaaaaaaaaaaaaaaaaaaaa

SEQ ID NO: 92 - protein, 296 aa, Homo sapiens
MEHLKAFDDEINAFLDNMFGPRDSRVRGWFTLDSYLPTFFLTVMYLLSIWLGNKYMKNRPALSLRGILTLYNLGITLLSAYMLAELILSTWEGGYNLQCQDLTSAGEADIRVAKVLWWYYFSKSVEFLDTIFFVLRKKTSQITFLHVYHHASMFNIWWCVLNWIPCGQSFFGPTLNSFVHILMYSYYGLSVFPSMHKYLWWKKYLTQAQLVQFVLTITHTMSAVVKPCGFPFGCLIFQSSYMLTLVILFLNFYVQTYRKKPMKKDMQEPPAGKEVKNGFSKAYFTAANGVMNKKAQ

SEQ ID NO: 93 - DNA, 4321 bp, Homo sapiens
cgagatcccggggagccagcttgctgggagagcgggacggtccggagcaagcccacaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcggcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttagaattccggcggagagaaccctctgttttcccccactctctctccacctcctcctgccttccccaccccgagtgcggagcagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaagcattggaaaccctatttccccaccccagctcatgccccctttcagatgtcttctgcctgttataactctgcactactcctctgcagtgccttggggaatttcctctattgatgtacagtctgtcatgaacatgttcctgaattctatttgctgggctttttttttctctttctctcctttctttttcttcttccctccctatctaaccctcccatggcaccttcagactttgcttcccattgtggctcctatctgtgttttgaatggtgttgtatgcctttaaatctgtgatgatcctcatatggcccagtgtcaagttgtgcttgtttacagcactactctgtgccagccacacaaacgtttacttatcttatgccacgggaagtttagagagctaagattatctggggaaatcaaaacaaaaaacaagcaaacaaaaaaaaaa

SEQ ID NO: 94 - protein, 919 aa, Homo sapiens
MEVQLGLGRVYPRPPSKTYRGAFQNLFQSVREVIQNPGPRHPEAASAAPPGASLLLLQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 95 - DNA, 2119 bp, Homo sapiens
ggcacgagtaggggtggcgggtcagtgctgctcgggggcttctccatccaggtccctggagttcctggtccctggagctccgcacttggcgcgcaacctgcgtgaggcagcgcgactctggcgactggccggccatgccttcccgggctgaggactatgaagtgttgtacaccattggcacaggctcctacggccgctgccagaagatccggaggaagagtgatggcaagatattagtttggaaagaacttgactatggctccatgacagaagctgagaaacagatgcttgtttctgaagtgaatttgcttcgtgaactgaaacatccaaacatcgttcgttactatgatcggattattgaccggaccaatacaacactgtacattgtaatggaatattgtgaaggaggggatctggctagtgtaattacaaagggaaccaaggaaaggcaatacttagatgaagagtttgttcttcgagtgatgactcagttgactctggccctgaaggaatgccacagacgaagtgatggtggtcataccgtattgcatcgggatcttaaaccagccaatgttttcctggatggcaagcaaaacgtcaagcttggagactttgggctagctagaatattaaaccatgacacgagttttgcaaaaacatttgttggcacaccttattacatgtctcctgaacaaatgaatcgcatgtcctacaatgagaaatcagatatctggtcattgggctgcttgctgtatgagttatgtgcattaatgcctccatttacagcttttagccagaaagaactcgctgggaaaatcagagaaggcaaattcaggcgaattccataccgttactctgatgaattgaatgaaattattacgaggatgttaaacttaaaggattaccatcgaccttctgttgaagaaattcttgagaaccctttaatagcagatttggttgcagacgagcaaagaagaaatcttgagagaagagggcgacaattaggagagccagaaaaatcgcaggattccagccctgtattgagtgagctgaaactgaaggaaattcagttacaggagcgagagcgagctctcaaagcaagagaagaaagattggagcagaaagaacaggagctttgtgttcgtgagagactagcagaggacaaactggctagagcagaaaatctgttgaagaactacagcttgctaaaggaacggaagttcctgtctctggcaagtaatccagaacttcttaatcttccatcctcagtaattaagaagaaagttcatttcagtggggaaagtaaagagaacatcatgaggagtgagaattctgagagtcagctcacatctaagtccaagtgcaaggacctgaagaaaaggcttcacgctgcccagctgcgggctcaagccctgtcagatattgagaaaaattaccaactgaaaagcagacagatcctgggcatgcgctagccaggtagagagacacagagctgtgtacaggatgtaatattaccaacctttaaagactgatattcaaatgctgtagtgttgaatacttggccccatgagccatgcctttctgtatagtacacatgatatttcggaattggttttactgttcttcagcaactattgtacaaaatgttcacatttaatttttctttcttcttttaagaacatattataaaaagaatactttcttggttgggcttttaatcctgtgtgtgattactagtaggaacatgagatgtgacattctaaatcttgggagaaaaaataatattaggaaaaaaatatttatgcaggaagagtagcactcactgaatagttttaaatgactgagtggtatgcttacaattgtcatgtctagatttaaattttaagtctgagattttaaatgtttttgagcttagaaaacccagttagatgcaatttggtcattaataccatgacatcttgcttataaatattccattgctctgtagttcaaatctgttagctttgtgaaaattcatcactgtgatgtttgtattctttttttttttctgtttaacagaatatgagctgtctgtcatttacctacttctttcccactaaataaaagaattcttcagtta

SEQ ID NO: 96 - protein, 445 aa, Homo sapiens
MPSRAEDYEVLYTIGTGSYGRCQKIRRKSDGKILVWKELDYGSMTEAEKQMLVSEVNLLRELKHPNIVRYYDRIIDRTNTTLYIVMEYCEGGDLASVITKGTKERQYLDEEFVLRVMTQLTLALKECHRRSDGGHTVLHRDLKPANVFLDGKQNVKLGDFGLARILNHDTSFAKTFVGTPYYMSPEQMNRMSYNEKSDIWSLGCLLYELCALMPPFTAFSQKELAGKIREGKFRRIPYRYSDELNEIITRMLNLKDYHRPSVEEILENPLIADLVADEQRRNLERRGRQLGEPEKSQDSSPVLSELKLKEIQLQERERALKAREERLEQKEQELCVRERLAEDKLARAENLLKNYSLLKERKFLSLASNPELLNLPSSVIKKKVHFSGESKENIMRSENSESQLTSKSKCKDLKKRLHAAQLRAQALSDIEKNYQLKSRQILGMR

SEQ ID NO: 97 - DNA, 12680 bp, Homo sapiens
cctcccgcctcagttcgcgccgcgcctcggcttggaacgcaggagcgccggctccgggagcccgagcggagccagccgcgcgcacagccagcggccgcgccggcgatgcggggccaccccgcgcccgccccagtcccggccccggcccccgcgggaaggggctgagctgcccgccgccgcccggatggcgagcctcgccgcgctcgccctcagcctgctcctgaggctgcagctgccgccactgcccggcgcccgggctcagagcgccccaggtggctgttcctttgatgagcactacagcaactgtggttatagtgtggctctagggaccaatgggttcacctgggagcagattaacacaacggagaaaccaatgctggaccaggcagtgcccacaggatctttcatgatggtgaacagctctgggagagcctctggccagaaggcccaccttctcctgccaaccctgaaggagaatgacacccactgcatcgacttccattactacttctccagccgtgacaggtccagcccaggggccttgaacgtctacgtgaaggtgaatggtggcccccaagggaaccctgtgtggaatgtgtccggggtcgtcactgagggctgggtgaaggcagagctcgccatcagcactttctggccacatttctatcaggtgatatttgaatccgtctcattgaagggtcatcctggctacatcgccgtggacgaggtccgggtccttgctcatccatgcagaaaagcacctcattttctgcgactccaaaacgtggaggtgaatgtggggcagaatgccacatttcagtgcattgctggtgggaagtggtctcagcatgacaagctttggctccagcaatggaatggcagggacacggccctgatggtcacccgtgtggtcaaccacaggcgcttctcagccacagtcagtgtggcagacactgcccagcggagcgtcagcaagtaccgctgtgtgatccgctctgatggtgggtctggtgtgtccaactacgcggagctgatcgtgaaagagcctcccacgcccattgctcccccagagctgctggctgtgggggccacatacctgtggatcaagccaaatgccaactccatcatcggggatggccccatcatcctgaaggaagtggaatatcgcaccaccacaggcacgtgggcagagacccacatagtcgactctcccaactataagctgtggcatctggaccccgatgttgagtatgagatccgagtgctcctcacacgaccaggtgaggggggtacgggaccgccaggggctcccctcaccaccaggaccaagtgtgcagatccggtacatggcccacagaacgtggaaatcgtagacatcagagcccggcagctgaccctgcagtgggagcccttcggctacgcggtgacccgctgccatagctacaacctcaccgtgcagtaccagtatgtgttcaaccagcagcagtacgaggccgaggaggtcatccagacctcctcccactacaccctgcgaggcctgcgccccttcatgaccatccggctgcgactcttgctgtctaaccccgagggccgaatggagagcgaggagctggtggtgcagactgaggaagacgttccaggagctgttcctctagaatccatccaaggggggccctttgaggagaagatctacatccagtggaaacctcccaatgagaccaatggggtcatcacgctctacgagatcaactacaaggctgtcggctcgctggacccaagtgctgacctctcgagccagagggggaaagtgttcaagctccggaatgaaacccaccacctctttgtgggtctgtacccagggaccacctattccttcaccatcaaggccagcacagcaaagggctttgggccccctgtcaccactcggattgccaccaaaatttcagctccatccatgcctgagtacgacacagacaccccattgaatgagacagacacgaccatcacagtgatgctgaaacccgctcagtcccggggagctcctgtcagtgtttatcagctggttgtcaaggaggagcgacttcagaagtcacggagggcagctgacattattgagtgcttttcggtgcccgtgagctatcggaatgcctccagcctcgattctctacactactttgctgctgagttgaagcctgccaacctgcctgtcacccagccatttacagtgggtgacaataagacatacaatggctactggaaccctcctctctctcccctgaaaagctacagcatctacttccaggcactcagcaaagccaatggagagaccaaaatcaactgtgttcgtctggctacaaaagcaccaatgggcagcgcccaggtgaccccggggactccactctgcctcctcaccacaggtgcctccacccagaattctaacactgtggagccagagaagcaggtggacaacaccgtgaagatggctggcgtgatcgctggcctcctcatgttcatcatcattctcctgggcgtgatgctcaccatcaaaaggagaagaaatgcttattcctactcctattacttgtcccaaaggaagctggccaagaagcagaaggagacccagagtggagcccagagggagatggggcctgtggcctctgccgacaaacccaccaccaagctcagcgccagccgcaatgatgaaggcttctcttctagttctcaggacgtcaacggattcacagatggcagccgcggggagctttcccagcccaccctcacgatccagactcatccctaccgcacctgtgaccctgtggagatgagctacccccgggaccagttccaactcgccatccgggtggctgacttgctgcagcacatcacgcagatgaagagaggccagggctacgggttcaaggaggaatacgaggccttaccagaggggcagacagcttcgtgggacacagccaaggaggatgaaaaccgcaataagaatcgatatgggaacatcatatcctacgaccattcccgggtgaggctgctggtgctggatggagacccgcactctgactacatcaatgccaactacattgacggataccatcgacctcggcactacattgcgactcaaggtccgatgcaggagactgtaaaggacttttggagaatgatctggcaggagaactccgccagcatcgtcatggtcacaaacctggtggaagtgggcagggtgaaatgtgtgcgatactggccagatgacacggaggtctacggagacattaaagtcaccctgattgaaacagagcccctggcagaatacgtcatacgcaccttcacagtccagaagaaaggctaccatgagatccgggagctccgcctcttccacttcaccagctggcctgaccacggcgttccctgctatgccactggccttctgggcttcgtccgccaggtcaagttcctcaaccccccggaagctgggcccatagtggtccactgcagtgctggggctgggcggactggctgcttcattgccattgacaccatgcttgacatggccgagaatgaaggggtggtggacatcttcaactgcgtgcgtgagctccgggcccaaagggtcaacctggtacagacagaggagcaatatgtgtttgtgcacgatgccatcctggaagcgtgcctctgtggcaacactgccatccctgtgtgtgagttccgttctctctactacaatatcagcaggctggacccccagacaaactccagccaaatcaaagatgaatttcagaccctcaacattgtgacaccccgtgtgcggcccgaggactgcagcattgggctcctgccccggaaccatgataagaatcgaagtatggacgtgctgcctctggaccgctgcctgcccttccttatctcagtggacggagaatccagcaattacatcaacgcagcactgatggatagccacaagcagcctgccgccttcgtggtcacccagcaccctctacccaacaccgtggcagacttctggaggctggtgttcgattacaactgctcctctgtggtgatgctgaatgagatggacactgcccagttctgtatgcagtactggcctgagaagacctccgggtgctatgggcccatccaggtggagttcgtctccgcagacatcgacgaggacatcatccacagaatattccgcatctgtaacatggcccggccacaggatggttatcgtatagtccagcacctccagtacattggctggcctgcctaccgggacacgcccccctccaagcgctctctgctcaaagtggtccgacgactggagaagtggcaggagcagtatgacgggagggagggacgtactgtggtccactgcctaaatgggggaggccgtagtggaaccttctgtgccatctgcagtgtgtgtgagatgatccagcagcaaaacatcattgacgtgttccacatcgtgaaaacactgcgtaacaacaaatccaacatggtggagaccctggaacagtataaatttgtatacgaggtggcactggaatatttaagctccttttagctcaatgggatggggaactgccggagtccagaggctgctgtgaccaagcccccttttgtgtgaatggcagtaactgggctcaggagctctgaggtggcaccctgcctgactccaaggagaagactggtggccctgtgttccacggggggctctgcaccttctgaggggtctcctgttgccgtgggagatgctgctccaaaaggcccaggcttccttttcaacctaaccagccacagccaagggcccaagcagaagtacacccacaagcaaggccttggatttctggctcccagaccacctgcttttgttctgagtttgtggatctcttggcaagccaactgtgcaggtgctggggagtgggaggctcccctgccctccttctccttaggagtggaggagatgtgtgttctgctcctctacgtcatggaaaagattgaggctcttgggggtcactgctctgctgccccctgcaacctccttcaggggcctctggcaccagacatttgcagtctggaccagtgtgaccttacgatgttccctaggccacaagagaggccccccatcctcacacctaacctgcatggggcttcgcccacaaccattctgtaccccttccccagcctgggccttgaccgtccagcattcactggccggccagctgtgtccacagcagtttttgataaaggtgttctttgcttttttgtgtggtcagtgggagggggtggaactgcagggaacttctctgctcctccttgtctttgtaaaaagggaccacctccctggggcagggcttgggctgacctgtaggatgtaacccctgtgtttctttggtggtagctttctttggaagagacaaacaagataagatttgattattttccaaagtgtatgtgaaaagaaactttcttttggagggtgtaaaatcttagtctcttatgtcaaaaagaagggggcgggggagtttgagtatgtacctctaagacaaatctctcgggccttttattttttcctggcaatgtccttaaaagctcccaccctgggacagcatgccactgagcaaggagagatgggtgagcctgaagatggtccctttggtttctggggcaaatagagcaccagctttgtgcataatttggatgtccaaatttgaactccttcctaaagaaacccagcagccaccttgaaaaaggccattgtggagcccattatactttgatttaaaataggccaagagaatcaggcctggagatctagggtcttgtccaaagtgtgagtgagtcaatgagagggaaccaacatttgctaagtctctactgtatgccagggatcatgcttggcactttccataggacatttcacacagtccttagaacccccaggagagagctactgacttgttatcatctccatttgatcatctcctccaatgaggaaacccacgcaccttccttagtaatgaaatcctgggttccaaaggggcaggtaatggcaatgagacttctccgtgctgttttcttcatcttctctaagccaagcaattattttatggagggaaaataaggccagaaacttctgagcagataactccacaaatggaaatttagtactttcttcctgatgccagttcttctgggaagcgcagaatttcagatatattttagtaacacattcccagctccccaggaaagccagtctcatctaatttcttagtcagtaaaaacaattccctgttccttcaggctatgaatggaccagccagggaaactctcgaccttgatctctagccagtgcttaggcccaatatctgacagcctcaggtgggctgggacctaggaagctccatcttgaaggctggtctagccccagacagggcatgaggggcagagaattcaagaaggtacagctttggccctcaagagcccactgtatgctggggaaatggaaccatggtgcagtagtgtggagtggatgagtgttccatgagcctaggagcaagaaagtctcttcggcctcgggcttcctggagaaggggacgtccattcctgctgggtcttaacaagcataaaaaggaaaaaaaggaaactcaggcaaagggatccatatgtgcaatggcaaagaaatgtgaaaaggcattgggagaagcagtctgggggaggccagcccagtgcgggcacagcacaacacggggagcagcaagagatgagccagggtccaggagacagatgcccatcgcgagtacagactttgtcctattggcaacaaggagtccatggagctttagagagatgcactcagcttcgtgttggccaagactccttctgggccaatggggctgcctcttttcctttcatcagacactgtgaaaacattcccttaagcgtgcactttttaatatcacatctatttgtctgtctgctcattgttttgttgctggaactaaatatgcaatggatcatgagactcagattctatgagaaacccagggtctctgctttaccacggagcagggtcaccaacccagatctccaggcccatgaggatggaacatgaaaggagccgacaaaagttgcttccattggcatgggctctggagctgtccagaagtccagggacaccagacttgatcaaggaagggctgtcactttagaggttcaaaaggaagtgcctcaaagcaaaggcaagcaaaggaaccccacgatgaacttgctcttttcctttgatgagcctctccccaggtgtatttcagcagaccccggggacccacccccactgggcctgctggcctccctcggctccagcccaatgccccagctggccttccccagcctgcaaggagcctgtagcatggcaaatctgcctgctgtatgctattttcttagatcttggtacatccagacaggatgagggtggagggagagctatttaacacaaatcctaagatttttttctgctcaggaaggggtgaaatagctggcagatacaaaagacagtggcttttatcattttaaatggtaggaatttaaggtgtgacttcagggagaaacaaacttgcaaaaaaaaaaaatctcaggccatgttggggtaacccagcaagggccagtgatgatttcccccagctcatccccttattttcccacaacccaaccattctctaaagcaggacagtgaataggtcttaggccagtgcacacaggaagaaattgaggcttatggatggggatgacttccctaagatcccatgggacaaggatgtggcaaggcttggatgagatggggcaccagtgcccaggaatttgaacattttcctttacccaggaaatctccggagccaacaccaccacccccagggggtctccccaccccaccccatttacagggtgagctcagcctgtcatgagcagaggaaaatattattaatgctctctgagtctttacaacaggagctcttacctcatagatgtgggctctgtttggggaagatgcaaggaagtaatgagaagcccaggaaatttctccacctgtgtttatggcctaaatagcttcaggatgtatcttagctgcactccaacattgcatcctttctggggtgaagaatctgggccaaccaggggtccttgggcctctagaaggccacagtaggcctctctttgtgggaatggaaggggacagtttgcttttagtgctggccctctctgtgggtgtggcctgcaaaggaaccaacagaccctatgctggggactctaacatgtgagctcattaaattcttccagcattctaaaggagggtttgtgattgtcaccatttactgatgaggaaactaaggctcctaggggagaaatcacttgcccacagttccacagctagtgagtgaatgaaccaggatttaaaccggttttttctcactacagagacaatatttttccaccattgtatctcacatttttcccaggaggttacccataacagaagagactagagtggaacagatacgtcagtggataaagctcaaagcaaacaacagtaagcttaaaattccttcatagtctcatgttttacgttcacaattcatgcaaaatttgcattccactttctgatttagccttgttggttttaatatgactctatgaatatttcaaaaaaaaatgtgctctgttcctcatgttgttctgttctgttcaccccgctatgacggaccctaggtcagctggtcttcagcttgaccctagaattgactctaggagcagtgaccctgctgcctcccagagccagttataggctcaagatcaagaccaactgaccttctcctaggcagctcctttggtgtgtgggtgctctgacctcactgttcatgaggggacctcaactaaggcatcttccagttgggtgctggaaggaacccattaactcacactagaatgatgaggatttgctcatctggcgtggagaaggatgagcccacaaaaccctaaagggaaaagagaagctggacacagctgtactcagcagattcctgaatgctaggctggaaagtggtgcctgttgtccaagtggagtcacatggttgctaatgtgggcaagtctgaggacacacttcatgagcagctggggtctggaaggctcctcactttaccctagccacacataattactgggtgcctacagcacctagcaccttggagggggcactattaggaaatcgagattactatggcacaattaattcctgggtaaggcatggggttgtggtggacagagctcagtctttagtttgaacgaaaacatacatacatgaaaaacatacatgaaaaaaggaccctcatcaacattagaaggggtagatttggagcactttaggcaggaaaacaggaacgcaaggccaggaaactggaacccagtgaatactcagaaccgaggatgcagatgacttatttagcaaaatggtcacttctgtgacatagctggagaaaggatgggtaacagcttgccagagccacttggaacaagggcaaatctcagtgtctggggcaaaagatgatgcatttccctctgacccatcatgtttattcatcctccactccccattgccacactagctcttgctgtaagtcctcaccaggatctacatttcctcgtcgctggtgggaaccccttagagtacatagaggtatcagtccagtaagactgctctacacaacagaagtgaggcccagggagtagcagccaggcccttatcctgttacctctgcaggagtgactgcccaacccagatccagagacattgaaggaaatgataattccttggtacctcactgccttgggacaaaatgaagaaagccacccttccttaggctgcagcttgccactcctgggctgggtaaacaggtcatcagcaccaggctcaaccaggagtaacattctggaagacatgggtgagcccaagaggaagcatgaacaggacgctgttcctaagtcatgtcaacaggttgtgctgggccaggatccccagggaaaaaaatggtcaacccaactggagggtaggttagaagaaaaaaaacataaacgtggatagtcatgtcatctcaaatccctgacttggcttccccattacttgacagtctgagctccttcttagcctgtgaccagcttcaaatcacagccaagtaaaacaaggaaataggaaaagtaaatccaactagaagagacaagctgagattcagatttgtttactcctcccatgcaaagtttccctgttggaggttttccatgtatacatgtctagaagtgatagaatgcaaggccttggctttgtcttgcagggatctgcctttgaggtcatagactgaacagcagggagagaggttagtggtggagtgtggggggagctgttctagctccagtttcttctgacacatttttcaggatcatggatctgatcctccgaagcacagcagagatatctaagccatatttgtgcacatgagcagactcttctagttttttagtaaccagggatgggcttttgcatggcactgactatagagatgtcttgtagagatcaagccagtcttttgcatcccacctgcccacctccagaagagatgggaaaaggtcatcaaagggcattcaccaactgaaatccactcatgaatgttaggtctctaaaaggaggcatcaacactcacaatggtagcctccaaacctagcatcccacctatctaagagctcaggggtggtccactggggcagatacaagggaagtgcaagggctcaggatgaaagaaaatctattgggaagagttttaggggcttgatcattatggggcttccttctatatctgagaactgctctgggtggtgagatgtggactctgatccttaattggaatgttcggagaatgagtgtctggtggccttgaagtgttggacagaaaagtatcagtataaaagcctggagctcagggtaattaatgtagttcatggttccttagtgagcaggactcttggatgtggaggagaaagggtcataggaagtaaaccaccaaaattacaaaattgagtctctgtacaattacttcagtgcctttgggcttatgaatacaaatcagtgggccttctctatgatggtccaacaaactctcagtgtccaccctgtccctgtatctcccatggaagatgaataatgtcaggtgttctttgggtcaaaggccccagggcagtctggaggcttagagggcagagtggtgtcattccatgtaaagttaggcttctgaggggtcaggcagaatatggtgtccatatcttccatagctctgcagattcttggatgaagtcaagcacagtttgctagacccaggtcactcctctgagtataactaggacccatgagtgaaacttaatagctgtaaggaagaacctgctgtctgccagagaggataagctgcccatctcagcagctgtctaaaagaaggcaggtgtctctttaaagggaagagaagcattggtgaaatggatttcaggtcacttccattccagatgggtgagatcttgtggagctgggatcatgtttgaactcattcatacctgtagagcacgaatccaagtagattgtgtttggtctgtacaggctgaagccccctgctctcccacccaagtgcccccactgagcaggccaacatgctgttgtggccacatatactgggctgatccaggctggttatcaccaaacagcaaaccatagggaacagctgctttgccatagacccaatacccatgtagatctctcatgagagcagccataactcagacccactgaccaacagggccatgagtgacagccagaaccagtgaaggtccaagtaggacacagagcagggcttttcttaccatacacattatctccagaggttatttctaccccactccctattcaaggcctgttggagcacactgcaaaagcaaaagcacagtaactcaatttacacatgattataatcatttccagtgcacacatttcatcaccaggtggatcctgagctagcccatgtaaatccgggttaacccatattggtaatcatactcaaaagcacttttcaccctacattctactagccaatcaaagacaaagagttgtggcctctaccattgccttggcttctggacaccctcacaagctatcccaaggttcccgctcaactccagggaggctgacatcttcacatccactgggcatataatattgcatgagaccaaagtctccacactctttgcagcctcctccatgaatcccaatggcctgcacttgtacagtttgggtgtttgatagataaagcacgtatgagaagagaaaacaaaataaatcaactttttaaaaaagccagcactgtgctgtcaatgttttttttttcttttcaattctagctcagaaaagcagaaggtaaataatgtcaggtcaatgaatatcagatatattttttgactgtacattacagtgaagtgtaatctttttacacctgcaagtccatcttatttattcttgtaaatgttccctgacaatgtttgtaatatggctgtgttaaaaaatctatacaataaagctgtgaccctg

SEQ ID NO: 98 - protein, 1444 aa, Homo sapiens
MASLAALALSLLLRLQLPPLPGARAQSAPGGCSFDEHYSNCGYSVALGTNGFTWEQINTTEKPMLDQAVPTGSFMMVNSSGRASGQKAHLLLPTLKENDTHCIDFHYYFSSRDRSSPGALNVYVKVNGGPQGNPVWNVSGVVTEGWVKAELAISTFWPHFYQVIFESVSLKGHPGYIAVDEVRVLAHPCRKAPHFLRLQNVEVNVGQNATFQCIAGGKWSQHDKLWLQQWNGRDTALMVTRVVNHRRFSATVSVADTAQRSVSKYRCVIRSDGGSGVSNYAELIVKEPPTPIAPPELLAVGATYLWIKPNANSIIGDGPIILKEVEYRTTTGTWAETHIVDSPNYKLWHLDPDVEYEIRVLLTRPGEGGTGPPGAPLTTRTKCADPVHGPQNVEIVDIRARQLTLQWEPFGYAVTRCHSYNLTVQYQYVFNQQQYEAEEVIQTSSHYTLRGLRPFMTIRLRLLLSNPEGRMESEELVVQTEEDVPGAVPLESIQGGPFEEKIYIQWKPPNETNGVITLYEINYKAVGSLDPSADLSSQRGKVFKLRNETHHLFVGLYPGTTYSFTIKASTAKGFGPPVTTRIATKISAPSMPEYDTDTPLNETDTTITVMLKPAQSRGAPVSVYQLVVKEERLQKSRRAADIIECFSVPVSYRNASSLDSLHYFAAELKPANLPVTQPFTVGDNKTYNGYWNPPLSPLKSYSIYFQALSKANGETKINCVRLATTGASTQNSNTVEPEKQVDNTVKMAGVIAGLLMFIIILLGVMLTIKRRRNAYSYSYYLSQRKLAKKQKETQSGAQREMGPVASADKPTTKLSASRNDEGFSSSSQDVNGFTDGSRGELSQPTLTIQTHPYRTCDPVEMSYPRDQFQLAIRVADLLQHITQMKRGQGYGFKEEYEALPEGQTASWDTAKEDENRNKNRYGNIISYDHSRVRLLVLDGDPHSDYINANYIDGYHRPRHYIATQGPMQETVKDFWRMIWQENSASIVMVTNLVEVGRVKCVRYWPDDTEVYGDIKVTLIETEPLAEYVIRTFTVQKKGYHEIRELRLFHFTSWPDHGVPCYATGLLGFVRQVKFLNPPEAGPIVVHCSAGAGRTGCFIAIDTMLDMAENEGVVDIFNCVRELRAQRVNLVQTEEQYVFVHDAILEACLCGNTAIPVCEFRSLYYNISRLDPQTNSSQIKDEFQTLNIVTPRVRPEDCSIGLLPRNHDKNRSMDVLPLDRCLPFLISVDGESSNYINAALMDSHKQPAAFVVTQHPLPNTVADFWRLVFDYNCSSVVMLNEMDTAQFCMQYWPEKTSGCYGPIQVEFVSADIDEDIIHRIFRICNMARPQDGYRIVQHLQYIGWPAYRDTPPSKRSLLKVVRRLEKWQEQYDGREGRTVVHCLNGGGRSGTFCAICSVCEMIQQQNIIDVFHIVKTLRNNKSNMVETLEQYKFVYEVALEYLSSF

SEQ ID NO: 99 - DNA, 803 bp, Homo sapiens
ccggcacgagaggagttgtgagtttccaagccccagctcactctgaccacttctctgcctgcccagcatcatgaagggccttgcagctgccctccttgtcctcgtctgcaccatggccctctgctcctgtgcacaagttggtaccaacaaagagctctgctgcctcgtctatacctcctggcagattccacaaaagttcatagttgactattctgaaaccagcccccagtgccccaagccaggtgtcatcctcctaaccaagagaggccggcagatctgtgctgaccccaataagaagtgggtccagaaatacatcagcgacctgaagctgaatgcctgaggggcctggaagctgcgagggcccagtgaacttggtgggcccaggagggaacaggagcctgagccagggcaatggccctgccaccctggaggccacctcttctaagagtcccatctgctatgcccagccacattaactaactttaatcttagtttatgcatcatatttcattttgaaattgatttctattgttgagctgcattatgaaattagtattttctctgacatctcatgacattgtctttatcatcctttcccctttcccttcaactcttcgtacattcaatgcatggatcaatcagtgtgattagctttctcagcagacattgtgccatatgtatcaaatgacaaatctttattgaatggttttgctcagcaccaccttttaatatattggcagtacttattatataaaaggtaaaccagcattctcactgtgaaaaaaaaaaaaaaaaaaaaaaaa

SEQ ID NO: 100 - protein, 89 aa, Homo sapiens
MKGLAAALLVLVCTMALCSCAQVGTNKELCCLVYTSWQIPQKFIVDYSETSPQCPKPGVILLTKRGRQICADPNKKWVQKYISDLKLNA

SEQ ID NO: 101 - DNA, 2398 bp, Homo sapiens
ccgcagaggagcctcggccaggctagccagggcgcccccagcccctccccaggccgcgagcgcccctgccgcggtgcctggcctcccctcccagactgcagggacagcacccggtaactgcgagtggagcggaggacccgagcggctgaggagagaggaggcggcggcttagctgctacggggtccggccggcgccctcccgaggggggctcaggaggaggaaggaggacccgtgcgagaatgcctctgccctggagccttgcgctcccgctgctgctctcctgggtggcaggtggtttcgggaacgcggccagtgcaaggcatcacgggttgttagcatcggcacgtcagcctggggtctgtcactatggaactaaactggcctgctgctacggctggagaagaaacagcaagggagtctgtgaagctacatgcgaacctggatgtaagtttggtgagtgcgtgggaccaaacaaatgcagatgctttccaggatacaccgggaaaacctgcagtcaagatgtgaatgagtgtggaatgaaaccccggccatgccaacacagatgtgtgaatacacacggaagctacaagtgcttttgcctcagtggccacatgctcatgccagatgctacgtgtgtgaactctaggacatgtgccatgataaactgtcagtacagctgtgaagacacagaagaagggccacagtgcctgtgtccatcctcaggactccgcctggccccaaatggaagagactgtctagatattgatgaatgtgcctctggtaaagtcatctgtccctacaatcgaagatgtgtgaacacatttggaagctactactgcaaatgtcacattggtttcgaactgcaatatatcagtggacgatatgactgtatagatataaatgaatgtactatggatagccatacgtgcagccaccatgccaattgcttcaatacccaagggtccttcaagtgtaaatgcaagcagggatataaaggcaatggacttcggtgttctgctatccctgaaaattctgtgaaggaagtcctcagagcacctggtaccatcaaagacagaatcaagaagttgcttgctcacaaaaacagcatgaaaaagaaggcaaaaattaaaaatgttaccccagaacccaccaggactcctacccctaaggtgaacttgcagcccttcaactatgaagagatagtttccagaggcgggaactctcatggaggtaaaaaagggaatgaagagaaaatgaaagaggggcttgaggatgagaaaagagaagagaaagccctgaagaatgacatagaggagcgaagcctgcgaggagatgtgtttttccctaaggtgaatgaagcaggtgaattcggcctgattctggtccaaaggaaagcgctaacttccaaactggaacataaagatttaaatatctcggttgactgcagcttcaatcatgggatctgtgactggaaacaggatagagaagatgattttgactggaatcctgctgatcgagataatgctattggcttctatatggcagttccggccttggcaggtcacaagaaagacattggccgattgaaacttctcctacctgacctgcaaccccaaagcaacttctgtttgctctttgattaccggctggccggagacaaagtcgggaaacttcgagtgtttgtgaaaaacagtaacaatgccctggcatgggagaagaccacgagtgaggatgaaaagtggaagacagggaaaattcagttgtatcaaggaactgatgctaccaaaagcatcatttttgaagcagaacgtggcaagggcaaaaccggcgaaatcgcagtggatggcgtcttgcttgtttcaggcttatgtccagatagccttttatctgtggatgactgaatgttactatctttatatttgactttgtatgtcagttccctggtttttttgatattgcatcataggacctctggcattttagaattactagctgaaaaattgtaatgtaccaacagaaatattattgtaagatgcctttcttgtataagatatgccaatatttgctttaaatatcatatcactgtatcttctcagtcatttctgaatctttccacattatattataaaatatggaaatgtcagtttatctcccctcctcagtatatctgatttgtataagtaagttgatgagcttctctctacaacatttctagaaaatagaaaaaaaagcacagagaaatgtttaactgtttgactcttatgatacttcttggaaactatgacatcaaagatagacttttgcctaagtggcttagctgggtctttcatagccaaacttgtatatttaaattctttgtaataataatatccaaatcatcaaaaaaaaaaaaaaaaa

SEQ ID NO: 102 - protein, 553 aa, Homo sapiens
MPLPWSLALPLLLSWVAGGFGNAASARHHGLLASARQPGVCHYGTKLACCYGWRRNSKGVCEATCEPGCKFGECVGPNKCRCFPGYTGKTCSQDVNECGMKPRPCQHRCVNTHGSYKCFCLSGHMLMPDATCVNSRTCAMINCQYSCEDTEEGPQCLCPSSGLRLAPNGRDCLDIDECASGKVICPYNRRCVNTFGSYYCKCHIGFELQYISGRYDCIDINECTMDSHTCSHHANCFNTQGSFKCKCKQGYKGNGLRCSAIPENSVKEVLRAPGTIKDRIKKLLAHKNSMKKKAKIKNVTPEPTRTPTPKVNLQPFNYEEIVSRGGNSHGGKKGNEEKMKEGLEDEKREEKALKNDIEERSLRGDVFFPKVNEAGEFGLILVQRKALTSKLEHKDLNISVDCSFNHGICDWKQDREDDFDWNPADRDNAIGFYMAVPALAGHKKDIGRLKLLLPDLQPQSNFCLLFDYRLAGDKVGKLRVFVKNSNNALAWEKTTSEDEKWKTGKIQLYQGTDATKSIIFEAERGKGKTGEIAVDGVLLVSGLCPDSLLSVDD

SEQ ID NO: 103 - DNA, 1120 bp, Homo sapiens
gagacattcctcaattgcttagacatattctgagcctacagcagaggaacctccagtctcagcaccatgaatcaaactgcgattctgatttgctgccttatctttctgactctaagtggcattcaaggagtacctctctctagaaccgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaatgtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtataagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaataaaaatgaggtactctcctggaaatattaag

SEQ ID NO: 104 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKEMSKRSP

SEQ ID NO: 105 - DNA, 6253 bp, Homo sapiens
gtaacaaccgtcaccctgggtcccgactgcccacctcctcctcctccccctccccccaacaacaacaacaacaacaactccaagcacaccggccataagagtgcgtgtgtccccaacatgaccgaacgaagaagggacgagctctctgaagagatcaacaacttaagagagaaggtcatgaagcagtcggaggagaacaacaacctgcagagccaggtgcagaagctcacagaggagaacaccacccttcgagagcaagtggaacccacccctgaggatgaggatgatgacatcgagctccgcggtgctgcagcagctgctgccccaccccctccaatagaggaagagtgcccagaagacctcccagagaagttcgatggcaacccagacatgctggctcctttcatggcccagtgccagatcttcatggaaaagagcaccagggatttctcagttgatcgtgtccgtgtctgcttcgtgacaagcatgatgaccggccgtgctgcccgttgggcctcagcaaagctggagcgctcccactacctgatgcacaactacccagctttcatgatggaaatgaagcatgtctttgaagaccctcagaggcgagaggttgccaaacgcaagatcagacgcctgcgccaaggcatggggtctgtcatcgactactccaatgctttccagatgattgcccaggacctggattggaacgagcctgcgctgattgaccagtaccacgagggcctcagcgaccacattcaggaggagctctcccacctcgaggtcgccaagtcgctgtctgctctgattgggcagtgcattcacattgagagaaggctggccagggctgctgcagctcgcaagccacgctcgccaccccgggcgctggtgttgcctcacattgcaagccaccaccaggtagatccaaccgagccggtgggaggtgcccgcatgcgcctgacgcaggaagaaaaagaaagacgcagaaagctgaacctgtgcctctactgtggaacaggaggtcactacgctgacaattgtcctgccaaggcctcaaagtcttcgccggcgggaaactccccggccccgctgtagagggaccttcagcgaccgggccagaaataataaggtccccacaagatgatgcctcatctccacacttgcaagtgatgctccagattcatcttccgggcagacacaccctgttcgtccgagccatgatcgattctggtgcttctggcaacttcattgatcacgaatatgttgctcaaaatggaattcctctaagaatcaaggactggccaatacttgtggaagcaattgatgggcgccccatagcatcgggcccagttgtccacgaaactcacgacctgatagttgacctgggagatcaccgagaggtgctgtcatttgatgtgactcagtctccattcttccctgtcgtcctaggggttcgctggctgagcacacatgatcccaatatcacatggagcactcgatctatcgtctttgattctgaatactgccgctaccactgccggatgtattctccaataccaccatcgctcccaccaccagcaccacaaccgccactctattatccagtagatggatacagagtttaccaaccagtgaggtattactatgtccagaatgtgtacactccagtagatgagcacgtctacccagatcaccgcctggttgaccctcacatagaaatgatacctggagcacacagtattcccagtggacatgtgtattcactgtccgaacctgaaatggcagctcttcgagattttgtggcaagaaatgtaaaagatgggctaattactccaacgattgcacctaatggagcccaagttctccaggtgaagagggggtggaaactgcaagtttcttatgattgccgagctccaaacaattttactatccagaatcagtatcctcgcctatctattccaaatttagaagaccaagcacacctggcaacgtacactgaattcgtacctcaaatacctggataccaaacataccccacatatgccgcgtacccgacctacccagtaggattcgcctggtacccagtgggacgagacggacaaggaagatcactatatgtacctgtgatgatcacttggaatccacactggtaccgccagcctccggtaccacagtacccgccgccacagccgccgcctccaccaccaccaccgccgccgcctccatcttacagtaccctgtaaatacctgtcatgtccttcaggatctctgccctcaaaatttattcctgttcagcttctcaatcagtgactgtgtgctaaattttaggctactgtatcttcaggccacctgaggcacatcctctctgaaacggctatggaaggttagggccactctggactggcacacatcctaaagcaccaaaagaccttcaacattttctgagagcaacagagtatttgccaataaatgatctctcatttttccaccttgactgccaatctaactaaaataattaataagtttactttccagccagtcctggaagtctgggttttacctgccaaaacctccatcaccatctaaattataggctgccaaatttgctgtttaacatttacagagaagctgatacaaacgcaggaaatgctgatttctttatggagggggagacgaggaggaggaggacatgacttttcttgcggtttcggtaccctctttttaaatcactggaggactgaggccttattaaggaagccaaaattatcggtgcagtgtggaaaggcttccgtgatcctctcgctgcacccttagaaacttcaccgtcttcaaactccatttccatggttctgttaattctcaaggagcagcaactcgactggttctcccaggagcaggaaaaacccttgtgacatgaaacatctcaggcctgaaaagaaagtgctctctcagatggactcttgcatgttaagactatgtcttcacatcatggtgcaaatcacatgtacccaatgactccggctttgacacaacaccttaccatcatcatgccatgatggcttccacaaagcattaaacctggtaaccagagattactggtggctccagcgttgttagatgttcatgaaatgtgaccacctctcaatcacctttgagggctaaagagtagcacatcaaaaggactccaaaatcccatacccaactcttaagagatttgtcctggtacttcagaaagaattttcatgagtgttcttaattggctggaaaagcaccagctgacgttttggaagaatctatccatgtgtctgcctccatatgcatctgggcatttcatcttcagtcccctcattagactgtagcattaggatgtgtggagagaggagaaatgatttagcacccagattcacactcctatgcctggaagggggacatctttgaagaagaggaattagggctgtggacactgtcttgaggatgtggacttccttagtgagctccacattacttgatggtaaccacttcaaaaggatcagaatccacgtaatgaaaaaggtccctctagaggatggagctgatgtgaagctgccaatggatgaaaagcctcagaaagcaactcaaaggactcaaagcaacggacaacacaagagttgtcttcagcccagtgacacctctgatgtcccctggaagctttgtgctaacctgggactgcctgacttcctttagcctggtcccttgctactaccttgaactgttttatctaacctctctttttctgtttaattctttgctactgccattgaccctgctgcaggatttgtgtcattttcctgcctggttgctgagactccattttgctgccacacacagagatgtaagaggcaggctttaattgccaaagcacagtttgagcagtagaaaacaacatggtgtatatctcaaattgcctgacatgaagaggagtctaacggtgaagtttcacttttcatcagcatcatctttcacatgttcattatcatccgctcttattcttgcatgtttaaacacttaaaatttttagtataatttttagtgtgttttgaagtggtgactaggctttcaaaaacttccattgaattacaaagcactatccagttcttattgttaaactaagtaaaaatgataagtaacatagtgtaaaatattcctttactgtgaacttcttacaatgctgtgaatgagaggctcctcagaactggagcatttgtataataattcatcctgttcatcttcaattttaacatcatatataatttcaattctatcaattgggcctttaaaaatcatataaaaggatataaaatttgaaaagagaaacctaattggctatttaatccaaaacaacttttttttttccttcaatggaatcagaaagcttgtcaatcactcatgtgttttagagtaattacttttaaaatggtgcatttgtgcttctgaactattttgaagagtcacttctgtttacctcaagtatcaattcatcctccatacatttgaattcaagttgttttttgtcaaatttacagttgtcaattgatcttcaagctgcagggtgcctagaaatgggccgttgtctgtagccctggcatgtgcacacggacatttgccaccactgcaagcaaaagtctggagaagttcaccaacgacaagaacgattagggaaaatatgctgctgtgggttaacaactcagaaagtccctgatccacatttggctgtttactaaagcttgtgattaactttttggcagtgtgtactatgctctattgctatatatgctatctataaatgtagatgttaaggataagtaattctaaatttattattctatagttttgaagtttggttaagtttcctttcactcaattgatttattttgttgttaatcaaatttatgttaattggatcctttaaattttttttggcattttccaacaaaaatggctttattcataagaaaggaaaaaaatcaatggaatttgatatctaaagaagttagaaagggagcaaaataaaaaacataaaggagatagatgaattagtaagcaaatcagtagtcgagtttttcaaactggcaaaattaattaattgacttttagcccaaatttacattgttaattaaatcaagaaggaagaagatctaagagctcccattgataggcaagcctagagagaactagctaaatttatcatgctaggatattgaaacacagaaagtttacatacatttatgaagggtcaatttagtttggacagtgaggtatttgtcttagtggaaaaaaggagaattagtctgatcaaatcgtgaagtaatacagtgaacttgcaggtgcacaaaataagagggccacatctatatggtgcagtctggaattctgtttaagtttgtaggtacctcttggacttctgaattgatccagttgtcatccaccacagacatctcacatcagatacagacagttccaagattgacaacagagaacaacctgctggaaagacctgggcagaaatggagagccctgcgggaaccatgctacattttcatctaaagagagaatgcacatctgatgagactgaaagttctttgttgttttagattgtagaatggtattgaattggtctgtggaaaattgcattgcttttatttctttgtgtaatcaagtttaagtaataggggatatataatcataagcattttagggtgggagggactattaagtaattttaagtgggtggggttatttagaatgttagaataatattatgtattagatatcgctataagtggacatgcgtacttacttgtaaccctttaccctataattgctatccttaaagatttcaaataaactcggagggaactgcagggagaccaacttatttagagcgaattggacatggataaaaaccccagtgggagaaagttcaaaggtgattagattaataatttaatagaggatgagtgacctctgataaattactgctagaatgaacttgtcaatgatggatggtaaattttcatggaagttataaaagtgataaataaaaacccttgcttttacccctgtcagtagccctcctcctaccactgaaccccattgcccctacccctccttctaactttattgctgtattctcttcactctatatttctctctatttgctaatattgcattgctgttacaataaaaattcaataaagatttagtggttaagtgc

SEQ ID NO: 106 - protein, 325 aa, Homo sapiens
MTERRRDELSEEINNLREKVMKQSEENNNLQSQVQKLTEENTTLREQVEPTPEDEDDDIELRGAAAAAAPPPPIEEECPEDLPEKFDGNPDMLAPFMAQCQIFMEKSTRDFSVDRVRVCFVTSMMTGRAARWASAKLERSHYLMHNYPAFMMEMKHVFEDPQRREVAKRKIRRLRQGMGSVIDYSNAFQMIAQDLDWNEPALIDQYHEGLSDHIQEELSHLEVAKSLSALIGQCIHIERRLARAAAARKPRSPPRALVLPHIASHHQVDPTEPVGGARMRLTQEEKERRRKLNLCLYCGTGGHYADNCPAKASKSSPAGNSPAPL

SEQ ID NO: 107 - DNA, 5000 bp, Homo sapiens
ggcacgagcagaagcaacaataattgtgaaaaatacttcagcagttatggactcatctgtcattcaaaggaaaaaagtagctgtcattggtggtggcttggttggctcattacaagcatgctttcttgcaaagaggaatttccagattgatgtatatgaagctagggaagatactcgagtggctaccttcacacgtggaagaagcattaacttagccctttctcatagaggacgacaagccttgaaagctgttggcctggaagatcagattgtatcccaaggtattcccatgagagcaagaatgatccactctctttcaggaaaaaagtctgcaattccctatgggacaaagtctcagtatattctttctgtaagcagagaaaatctaaacaaggatctattgactgctgctgagaaataccccaatgtgaaaatgcactttaaccacaggctgttgaaatgtaatccagaggaaggaatgatcacagtgcttggatctgacaaagttcccaaagatgtcacttgtgacctcattgtaggatgtgatggagcctattcaactgtcagatctcacctgatgaagaaacctcgctttgattacagtcagcagtacattcctcatgggtacatggagttgactattccacctaagaacggagattatgccatggaacctaattatctgcatatttggcctagaaatacctttatgatgattgcacttcctaacatgaacaaatcattcacatgtactttgttcatgccctttgaagagtttgaaaaacttctaaccagtaatgatgtggtagatttcttccagaaatactttccggatgccatccctctaattggagagaaactcctagtgcaagatttcttcctgttgcctgcccagcccatgatatctgtaaagtgctcttcatttcactttaaatctcactgtgtactgctgggagatgcagctcatgctatagtgccgttttttgggcaaggaatgaatgcgggctttgaagactgcttggtatttgatgagttaatggataaattcagtaacgaccttagtttgtgtcttcctgtgttctcaagattgagaatcccagatgatcacgcgatttcagacctatccatgtacaattacatagagatgcgagcacatgtcaactcaagctggttcatttttcagaagaacatggagagatttcttcatgcgattatgccatcgacctttatccctctctatacaatggtcactttttccagaataagataccatgaggctgtgcagcgttggcattggcaaaaaaaggtgataaacaaaggactctttttcttgggatcactgatagccatcagcagtacctacctacttatacactacatgtcaccacgatctttcctctgcttgagaagaccatggaactggatagctcacttccggaatacaacatgtttccccgcaaaggccgtggactccctagaacaaatttccaatctcattagcaggtgatagaaaggttttgtggtagcaaatgcatgatttctctgtgaccaaaattaagcatgaaaaaaatgtttccattgccatatttgattcactagtggaagatagtgttctgcttataattaaactgaatgtagagtatctctgtatgttaattgcaattactggttggggggtgcattttaaaagatgaaacatgcagcttccctacattacacacactcaggttgagtcattctaactataaaagtgcaatgactaagatccttcacttctctgaaagtaaggccctagatgcctcagggaagacagtaatcatgccttttctttaaaagacacaataggactcgcaacagcattgactcaacacctaggactaaaaatcacaacttaactagcatgttaactgcacttttcattacgtgaatggaacttacctaaccacagggctcagacttactagataaaaccagaaatggaaataaggaattcaggggagttccagagacttacaaaatgaactcattttattttcccaccttcaaatataagtattatcatctatctgtttatcgtctatctatctatcatctatctatctatctatcatctatctatctatctatctatctatctatctatctatctatctatctatctctatttatttatgtatttagagatcaggtctcactctgttgaccaggctggagtgcagtggtgagatctgggttcactgcaacctctgcctcctgggctcaagcaatcctcccacttcagcctcccaaatagctggggctaccatggtatttttcagtagagaccgggtcttgccatgctgcccaggccagtctcaaactcctggcctcatgtgatctgcccacctcagcctcccaaagtacagggattagagttgtgagccaccgctgccagcccagagttaccctctaaagataagaaaaaggctattaatatcatactaagtgaaggacaggaaagggttttattcataaattaaatgtctacatgtgccagaatggaaaggaaacaaggggagacaacttttatagaaatacaaagccattactttattcaatttcagaccctcagaagcaatttactaatttattcttcgactacatactgcagcagaaccagcaatacacttgatttttaaaagcacatttagtgaaatgttttctttggttcatccttctttaacaggctgctgagtcactcagaaatccttcaaacatgattaattatgaagatgaaacactagagtcatataagaaataaaaattgggcaataaaataaaatgattcagtgtttcttttctatattgtcaatgaaaaccttgagttctaataatccatgttcagtttgtagggaaagaaaaaataatttttccttctacccactttaggttccttggctggggcccctataacaaaagacagattgacaagagaaaaacaaacataaatttattagcgggtatatgtaatatatatgtgggaaatacaggggaatgagcaaatctcaaagagctggcgtcttagaactccctggcttatatagcatcgacaaagaacagtaaatttttagagaaacaacaaaacaaagaaaaagagctttgagtctgtaggggcagcaatttgggggaagcaaatatatgggagtttgccttgtagattcctctggtggtggtctccaggctgacaaggattcaaagttgtctctgaaactcctctttgtcatactgcacatataaaacgtcttttgtttccaacaagaggatttctttttcattctagaattatctccttgataacttgatcagatataggacatgacactgaatagagtccaacagtacaaaaaaaattcagtatgttctagctacttcacacatgtgtacgcgacagttatttttacagtaaggtattttcgagaaaaatgcattacgtgttttggaaaatagagtaatttaaaaaatatatttgaaatgaaaatctccaacacattagaagatgatgatgttagatgcccatcgtgtgccacaagtggttttttcattatgtaaagcacccgttgaattaaaagaatttgtttttgttcaacctcttcctgaggcccaagagcatatgggcaattcggatttcctgctggaccacaaggttctgttgatattacatagaaacgggtattccagacacttcttatgatgaaagtccaaaagtggcatccaatttaaggccccatctttcgttgccattcttcattcctacaaaggacgaacttggattacatcaactttggacccattggttttgtcgctgtcgtcaactgacagtgattcaccactggtgatgataaaaatgatggaagaagagttgaaagtcacttttttctttggcctgtccccatctttctgtgacatcacaatgggtctgatctgcatttcacttccagctgctggtaggtctttagcaggcctctggcacctcagcagtcggaggcacagaagctgcaaaagggatcttcgaaactgggcagagaaaaaataaagtggaatattaagtaaaagttgggcactaatctggattaacattcgaggaaatcagttgagctgatttaagttgttttttgtttgttagcaggtgtggatgtggggttatgtggtcatgctcagatctacctaaatcaccccagagctttatgtcttttattcattctaaatcttattaaccggaatatgtaggaccatttcaataccttgtaatcctccaagcttcaatctgcacacactttctatgagggcaggtacaactattaagagattttgaacattaagttagtccacaaatattcagtgggcatctactaggtgacagccactgtgctataattagagactttttactataagcatcaaaaacagataaggctcttcctggcagagtttacagcctggtgtacttgctaatgtctctttaattaggtgaagaattttttttttctatcgaaattactaatcagttggggaaaaaaatactatagcagacagcactaatgtcatcaacaaacattgttcttctccgtgtcctgggtacaacatcgaataatatttcttggcctcctttccgcttctcctctctgctgttcctctctacaagaacctgggaggccaacgcctaaagatcataatatcacaatggaaggaacctagattcctaaatgactgcataggacagatcccatctcctccacccaatacattattagactgaactgtgacctgaaatgagcaataaactctgtattaattcactgaaatgttggggttgcttgttatagtagtcggtccatcatgaccagtaaaacataaatcaaaagttaatgtaattgttatcccattatttagagcgaaataaatgttgaatatatggactttctcagattaggaaataccaattaaaaatataataaatagct

SEQ ID NO: 108 - protein, 486 aa, Homo sapiens
MDSSVIQRKKVAVIGGGLVGSLQACFLAKRNFQIDVYEAREDTRVATFTRGRSINLALSHRGRQALKAVGLEDQIVSQGIPMRARMIHSLSGKKSAIPYGTKSQYILSVSRENLNKDLLTAAEKYPNVKMHFNHRLLKCNPEEGMITVLGSDKVPKDVTCDLIVGCDGAYSTVRSHLMKKPRFDYSQQYIPHGYMELTIPPKNGDYAMEPNYLHIWPRNTFMMIALPNMNKSFTCTLFMPFEEFEKLLTSNDVVDFFQKYFPDAIPLIGEKLLVQDFFLLPAQPMISVKCSSFHFKSHCVLLGDAAHAIVPFFGQGMNAGFEDCLVFDELMDKFSNDLSLCLPVFSRLRIPDDHAISDLSMYNYIEMRAHVNSSWFIFQKNMERFLHAIMPSTFIPLYTMVTFSRIRYHEAVQRWHWQKKVINKGLFFLGSLIAISSTYLLIHYMSPRSFLCLRRPWNWIAHFRNTTCFPAKAVDSLEQISNLISR

SEQ ID NO: 109 - DNA, 2148 bp, Homo sapiens
gcttcagggtacagctcccccgcagccagaagccgggcctgcagcccctcagcaccgctccgggacaccccacccgcttcccaggcgtgacctgtcaacagcaacttcgcggtgtggtgaactctctgaggaaaaaccattttgattattactctcagacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacaaccttaagcttctacgggaattccatctccatatctgccttgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaactagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttggggataggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagcaaaaaaaaaaaaaaaaaa

SEQ ID NO: 110 - protein, 509 aa, Homo sapiens
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 111 - DNA, 2740 bp, Homo sapiens
cgctgccatgcggctggcgctgctctgggccctggggctcctgggcgcgggcagccctctgccttcctggccgctcccaaatataggtggcactgaggagcagcaggcagagtcagagaaggccccgagggagcccttggagccccaggtccttcaggacgatctcccaattagcctcaaaaaggtgcttcagaccagtctgcctgagcccctgaggatcaagttggagctggacggtgacagtcatatcctggagctgctacagaatagggagttggtcccaggccgcccaaccctggtgtggtaccagcccgatggcactcgggtggtcagtgagggacacactttggagaactgctgctaccagggaagagtgcggggatatgcaggctcctgggtgtccatctgcacctgctctgggctcagaggcttggtggtcctgaccccagagagaagctataccctggagcaggggcctggggaccttcagggtcctcccattatttcgcgaatccaagatctccacctgccaggccacacctgtgccctgagctggcgggaatctgtacacactcagacgccaccagagcaccccctgggacagcgccacattcgccggaggcgggatgtggtaacagagaccaagactgtggagttggtgattgtggctgatcactcggaggcccagaaataccgggacttccagcacctgctaaaccgcacactggaagtggccctcttgctggacacattcttccggcccctgaatgtacgagtggcactagtgggcctggaggcctggacccagcgtgacctggtggagatcagcccaaacccagctgtcaccctcgaaaacttcctccactggcgcagggcacatttgctgcctcgattgccccatgacagtgcccagctggtgactggtacttcattctctgggcctacggtgggcatggccattcagaactccatctgttctcctgacttctcaggaggtgtgaacatggaccactccaccagcatcctgggagtcgcctcctccatagcccatgagttgggccacagcctgggcctggaccatgatttgcctgggaatagctgcccctgtccaggtccagccccagccaagacctgcatcatggaggcctccacagacttcctaccaggcctgaacttcagcaactgcagccgacgggccctggagaaagccctcctggatggaatgggcagctgcctcttcgaacggctgcctagcctaccccctatggctgctttctgcggaaatatgtttgtggagccgggcgagcagtgtgactgtggcttcctggatgactgcgtcgatccctgctgtgattctttgacctgccagctgaggccaggtgcacagtgtgcatctgacggaccctgttgtcaaaattgccagctgcgcccgtctggctggcagtgtcgtcctaccagaggggattgtgacttgcctgaattctgcccaggagacagctcccagtgtccccctgatgtcagcctaggggatggcgagccctgcgctggcgggcaagctgtgtgcatgcacgggcgttgtgcctcctatgcccagcagtgccagtcactttggggacctggagcccagcccgctgcgccactttgcctccagacagctaatactcggggaaatgcttttgggagctgtgggcgcaaccccagtggcagttatgtgtcctgcacccctagagatgccatttgtgggcagctccagtgccagacaggtaggacccagcctctgctgggctccatccgggatctactctgggagacaatagatgtgaatgggactgagctgaactgcagctgggtgcacctggacctgggcagtgatgtggcccagcccctcctgactctgcctggcacagcctgtggccctggcctggtgtgtatagaccatcgatgccagcgtgtggatctcctgggggcacaggaatgtcgaagcaaatgccatggacatggggtctgtgacagcaacaggcactgctactgtgaggagggctgggcaccccctgactgcaccactcagctcaaagcaaccagctccctgaccacagggctgctcctcagcctcctggtcttattggtcctggtgatgcttggtgccggctactggtaccgtgcccgcctgcaccagcgactctgccagctcaagggacccacctgccagtacagggcagcccaatctggtccctctgaacggccaggacctccgcagagggccctgctggcacgaggcactaagtctcaggggccagccaagcccccacccccaaggaagccactgcctgccgacccccagggccggtgcccatcgggtgacctgcccggcccaggggctggaatcccgcccctagtggtaccctccagaccagcgccaccgcctccgacagtgtcctcgctctacctctgacctctccggaggttccgctgcctccaagccggacttagggcttcaagaggcgggcgtgccctctggagtcccctaccatgactgaaggcgccagagactggcggtgtcttaagactccgggcaccgccacgcgctgtcaagcaacactctgcggacctgccggcgtagttgcagcgggggcttggggaggggctgggggttggacgggattgaggaaggtccgcacagcctgtctctgctcagttgcaataaacgtgacatcttgggagcgttaaaaaaaaaaaaaaa

SEQ ID NO: 112 - protein, 814 aa, Homo sapiens
MRLALLWALGLLGAGSPLPSWPLPNIGGTEEQQAESEKAPREPLEPQVLQDDLPISLKKVLQTSLPEPLRIKLELDGDSHILELLQNRELVPGRPTLVWYQPDGTRVVSEGHTLENCCYQGRVRGYAGSWVSICTCSGLRGLVVLTPERSYTLEQGPGDLQGPPIISRIQDLHLPGHTCALSWRESVHTQTPPEHPLGQRHIRRRRDVVTETKTVELVIVADHSEAQKYRDFQHLLNRTLEVALLLDTFFRPLNVRVALVGLEAWTQRDLVEISPNPAVTLENFLHWRRAHLLPRLPHDSAQLVTGTSFSGPTVGMAIQNSICSPDFSGGVNMDHSTSILGVASSIAHELGHSLGLDHDLPGNSCPCPGPAPAKTCIMEASTDFLPGLNFSNCSRRALEKALLDGMGSCLFERLPSLPPMAAFCGNMFVEPGEQCDCGFLDDCVDPCCDSLTCQLRPGAQCASDGPCCQNCQLRPSGWQCRPTRGDCDLPEFCPGDSSQCPPDVSLGDGEPCAGGQAVCMHGRCASYAQQCQSLWGPGAQPAAPLCLQTANTRGNAFGSCGRNPSGSYVSCTPRDAICGQLQCQTGRTQPLLGSIRDLLWETIDVNGTELNCSWVHLDLGSDVAQPLLTLPGTACGPGLVCIDHRCQRVDLLGAQECRSKCHGHGVCDSNRHCYCEEGWAPPDCTTQLKATSSLTTGLLLSLLVLLVLVMLGAGYWYRARLHQRLCQLKGPTCQYRAAQSGPSERPGPPQRALLARGTKSQGPAKPPPPRKPLPADPQGRCPSGDLPGPGAGIPPLVVPSRPAPPPPTVSSLYL

SEQ ID NO: 113 - DNA, 2545 bp, Homo sapiens
atccaatacaggagtgacttggaactccattctatcactatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataagagaccacttcaccaataagtattctgtgttaaaaatgttctattttaattataccgctatcattccaaaggaggatggcatataatacaaaggcttattaatttgactagaaaatttaaaacattactctgaaattgtaactaaagttagaaagttgattttaagaatccaaacgttaagaattgttaaaggctatgattgtctttgttcttctaccacccaccagttgaatttcatcatgcttaaggccatgattttagcaatacccatgtctacacagatgttcacccaaccacatcccactcacaacagctgcctggaagagcagccctaggcttccacgtactgcagcctccagagagtatctgaggcacatgtcagcaagtcctaagcctgttagcatgctggtgagccaagcagtttgaaattgagctggacctcaccaagctgctgtggccatcaacctctgtatttgaatcagcctacaggcctcacacacaatgtgtctgagagattcatgctgattgttattgggtatcaccactggagatcaccagtgtgtggctttcagagcctcctttctggctttggaagccatgtgattccatcttgcccgctcaggctgaccactttatttctttttgttcccctttgcttcattcaagtcagctcttctccatcctaccacaatgcagtgcctttcttctctccagtgcacctgtcatatgctctgatttatctgagtcaactcctttctcatcttgtccccaacaccccacagaagtgctttcttctcccaattcatcctcactcagtccagcttagttcaagtcctgcctcttaaataaacctttttggacacacaaattatcttaaaactcctgtttcacttggttcagtaccacatgggtgaacactcaatggttaactaattcttgggtgtttatcctatctctccaaccagattgtcagctccttgagggcaagagccacagtatatttccctgtttcttccacagtgcctaataatactgtggaactaggttttaataattttttaattgatgttgttatgggcaggatggcaaccagaccattgtctcagagcaggtgctggctctttcctggctactccatgttggctagcctctggtaacctcttacttattatcttcaggacactcactacagggaccagggatgatgcaacatccttgtctttttatgacaggatgtttgctcagcttctccaacaataagaagcacgtggtaaaacacttgcggatattctggactgtttttaaaaaatatacagtttaccgaaaatcatataatcttacaatgaaaaggactttatagatcagccagtgaccaaccttttcccaaccatacaaaaattccttttcccgaaggaaaagggctttctcaataagcctcagctttctaagatctaacaagatagccaccgagatccttatcgaaactcattttaggcaaatatgagttttattgtccgtttacttgtttcagagtttgtattgtgattatcaattaccacaccatctcccatgaagaaagggaacggtgaagtactaagcgctagaggaagcagccaagtcggttagtggaagcatgattggtgcccagttagcctctgcaggatgtggaaacctccttccaggggaggttcagtgaattgtgtaggagaggttgtctgtggccagaatttaaacctatactcactttcccaaattgaatcactgctcacactgctgatgatttagagtgctgtccggtggagatcccacccgaacgtcttatctaatcatgaaactccctagttccttcatgtaacttccctgaaaaatctaagtgtttcataaatttgagagtctgtgacccacttaccttgcatctcacaggtagacagtatataactaacaaccaaagactacatattgtcactgacacacacgttataatcatttatcatatatatacatacatgcatacactctcaaagcaaataatttttcacttcaaaacagtattgacttgtataccttgtaatttgaaatattttctttgttaaaatagaatggtatcaataaatagaccattaatcag

SEQ ID NO: 114 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 115 - DNA, 1695 bp, Homo sapiens
caagcaggatacgtttttctgttgggcattgactagattgtttgcaaaagtttcgcatcaaaaacaaacaacaacaacaaaaaaccaaacaactctccttgatctatactttgagaattgttgatttctttttttttattctgacttttaaaaacaacttttttttccacttttttaaaaaatgcactactgtgtgctgagcgcttttctgatcctgcatctggtcacggtcgcgctcagcctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaaatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccggcggaagaagcgtgctttggatgcggcctattgctttagaaatgtgcaggataattgctgcctacgtccactttacattgatttcaagagggatctagggtggaaatggatacacgaacccaaagggtacaatgccaacttctgtgctggagcatgcccgtatttatggagttcagacactcagcacagcagggtcctgagcttatataataccataaatccagaagcatctgcttctccttgctgcgtgtcccaagatttagaacctctaaccattctctactacattggcaaaacacccaagattgaacagctttctaatatgattgtaaagtcttgcaaatgcagctaaaattcttggaaaagtggcaagaccaaaatgacaatgatgatgataatgatgatgacgacgacaacgatgatgcttgtaacaagaaaacataagagagccttggttcatcagtgttaaaaaatttttgaaaaggcggtactagttcagacactttggaagtttgtgttctgtttgttaaaactggcatctgacacaaaaaaagttgaaggccttattctacatttcacctactttgtaagtgagagagacaagaagcaaatttttttaaa

SEQ ID NO: 116 - protein, 414 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 117 - DNA, 2439 bp, Homo sapiens
cagcacccagctccccgccaccgccatggtccccgacaccgcctgcgttcttctgctcaccctggctgccctcggcgcgtccggacagggccagagcccgttgggctcagacctgggcccgcagatgcttcgggaactgcaggaaaccaacgcggcgctgcaggacgtgcgggactggctgcggcagcaggtcagggagatcacgttcctgaaaaacacggtgatggagtgtgacgcgtgcgggatgcagcagtcagtacgcaccggcctacccagcgtgcggcccctgctccactgcgcgcccggcttctgcttccccggcgtggcctgcatccagacggagagcggcggccgctgcggcccctgccccgcgggcttcacgggcaacggctcgcactgcaccgacgtcaacgagtgcaacgcccacccctgcttcccccgagtccgctgtatcaacaccagcccggggttccgctgcgaggcttgcccgccggggtacagcggccccacccaccagggcgtggggctggctttcgccaaggccaacaagcaggtttgcacggacatcaacgagtgtgagaccgggcaacataactgcgtccccaactccgtgtgcatcaacacccggggctccttccagtgcggcccgtgccagcccggcttcgtgggcgaccaggcgtccggctgccagcgcggcgcacagcgcttctgccccgacggctcgcccagcgagtgccacgagcatgcagactgcgtcctagagcgcgatggctcgcggtcgtgcgtgtgtcgcgttggctgggccggcaacgggatcctctgtggtcgcgacactgacctagacggcttcccggacgagaagctgcgctgcccggagccgcagtgccgtaaggacaactgcgtgactgtgcccaactcagggcaggaggatgtggaccgcgatggcatcggagacgcctgcgatccggatgccgacggggacggggtccccaatgaaaaggacaactgcccgctggtgcggaacccagaccagcgcaacacggacgaggacaagtggggcgatgcgtgcgacaactgccggtcccagaagaacgacgaccaaaaggacacagaccaggacggccggggcgatgcgtgcgacgacgacatcgacggcgaccggatccgcaaccaggccgacaactgccctagggtacccaactcagaccagaaggacagtgatggcgatggtataggggatgcctgtgacaactgtccccagaagagcaacccggatcaggcggatgtggaccacgactttgtgggagatgcttgtgacagcgatcaagaccaggatggagacggacatcaggactctcgggacaactgtcccacggtgcctaacagtgcccaggaggactcagaccacgatggccagggtgatgcctgcgacgacgacgacgacaatgacggagtccctgacagtcgggacaactgccgcctggtgcctaaccccggccaggaggacgcggacagggacggcgtgggcgacgtgtgccaggacgactttgatgcagacaaggtggtagacaagatcgacgtgtgtccggagaacgctgaagtcacgctcaccgacttcagggccttccagacagtcgtgctggacccggagggtgacgcgcagattgaccccaactgggtggtgctcaaccagggaagggagatcgtgcagacaatgaacagcgacccaggcctggctgtgggttacactgccttcaatggcgtggacttcgagggcacgttccatgtgaacacggtcacggatgacgactatgcgggcttcatctttggctaccaggacagctccagcttctacgtggtcatgtggaagcagatggagcaaacgtattggcaggcgaaccccttccgtgctgtggccgagcctggcatccaactcaaggctgtgaagtcttccacaggccccggggaacagctgcggaacgctctgtggcatacaggagacacagagtcccaggtgcggctgctgtggaaggacccgcgaaacgtgggttggaaggacaagaagtcctatcgttggttcctgcagcaccggccccaagtgggctacatcagggtgcgattctatgagggccctgagctggtggccgacagcaacgtggtcttggacacaaccatgcggggtggccgcctgggggtcttctgcttctcccaggagaacatcatctgggccaacctgcgttaccgctgcaatgacaccatcccagaggactatgagacccatcagctgcggcaagcctagggaccagggtgaggacccgccggatgacagccaccctcaccgcggctggatgggggctctgcacccagcccaaggggtggccgtcctgagggggaagtgagaagggctcagagaggacaaaataaagtgtgtgtgcaggg

SEQ ID NO: 118 - protein, 757 aa, Homo sapiens
MVPDTACVLLLTLAALGASGQGQSPLGSDLGPQMLRELQETNAALQDVRDWLRQQVREITFLKNTVMECDACGMQQSVRTGLPSVRPLLHCAPGFCFPGVACIQTESGGRCGPCPAGFTGNGSHCTDVNECNAHPCFPRVRCINTSPGFRCEACPPGYSGPTHQGVGLAFAKANKQVCTDINECETGQHNCVPNSVCINTRGSFQCGPCQPGFVGDQASGCQRGAQRFCPDGSPSECHEHADCVLERDGSRSCVCRVGWAGNGILCGRDTDLDGFPDEKLRCPEPQCRKDNCVTVPNSGQEDVDRDGIGDACDPDADGDGVPNEKDNCPLVRNPDQRNTDEDKWGDACDNCRSQKNDDQKDTDQDGRGDACDDDIDGDRIRNQADNCPRVPNSDQKDSDGDGIGDACDNCPQKSNPDQADVDHDFVGDACDSDQDQDGDGHQDSRDNCPTVPNSAQEDSDHDGQGDACDDDDDNDGVPDSRDNCRLVPNPGQEDADRDGVGDVCQDDFDADKVVDKIDVCPENAEVTLTDFRAFQTVVLDPEGDAQIDPNWVVLNQGREIVQTMNSDPGLAVGYTAFNGVDFEGTFHVNTVTDDDYAGFIFGYQDSSSFYVVMWKQMEQTYWQANPFRAVAEPGIQLKAVKSSTGPGEQLRNALWHTGDTESQVRLLWKDPRNVGWKDKKSYRWFLQHRPQVGYIRVRFYEGPELVADSNVVLDTTMRGGRLGVFCFSQENIIWANLRYRCNDTIPEDYETHQLRQA

SEQ ID NO: 119 - DNA, 3264 bp, Homo sapiens
gggacagggctgaggatgaggagaaccctggggacccagaagaccgtgccttgcccggaagtcctgcctgtaggcctgaaggacttgccctaacagagcctcaacaactacctggtgattcctacttcagccccttggtgtgagcagcttctcaacatgaactacagcctccacttggccttcgtgtgtctgagtctcttcactgagaggatgtgcatccaggggagtcagttcaacgtcgaggtcggcagaagtgacaagctttccctgcctggctttgagaacctcacagcaggatataacaaatttctcaggcccaattttggtggagaacccgtacagatagcgctgactctggacattgcaagtatctctagcatttcagagagtaacatggactacacagccaccatatacctccgacagcgctggatggaccagcggctggtgtttgaaggcaacaagagcttcactctggatgcccgcctcgtggagttcctctgggtgccagatacttacattgtggagtccaagaagtccttcctccatgaagtcactgtgggaaacaggctcatccgcctcttctccaatggcacggtcctgtatgccctcagaatcacgacaactgttgcatgtaacatggatctgtctaaataccccatggacacacagacatgcaagttgcagctggaaagctggggctatgatggaaatgatgtggagttcacctggctgagagggaacgactctgtgcgtggactggaacacctgcggcttgctcagtacaccatagagcggtatttcaccttagtcaccagatcgcagcaggagacaggaaattacactagattggtcttacagtttgagcttcggaggaatgttctgtatttcattttggaaacctacgttccttccactttcctggtggtgttgtcctgggtttcattttggatctctctcgattcagtccctgcaagaacctgcattggagtgacgaccgtgttatcaatgaccacactgatgatcgggtcccgcacttctcttcccaacaccaactgcttcatcaaggccatcgatgtgtacctggggatctgctttagctttgtgtttggggccttgctagaatatgcagttgctcactacagttccttacagcagatggcagccaaagatagggggacaacaaaggaagtagaagaagtcagtattactaatatcatcaacagctccatctccagctttaaacggaagatcagctttgccagcattgaaatttccagcgacaacgttgactacagtgacttgacaatgaaaaccagcgacaagttcaagtttgtcttccgagaaaagatgggcaggattgttgattatttcacaattcaaaaccccagtaatgttgatcactattccaaactactgtttcctttgatttttatgctagccaatgtattttactgggcatactacatgtatttttgagtcaatgttaaatttcttgcatgccataggtcttcaacaggacaagataatgatgtaaatggtattttaggccaagtgtgcacccacatccaatggtgctacaagtgactgaaataatatttgagtctttctgctcaaagaatgaagctccaaccattgttctaagctgtgtagaagtcctagcattataggatcttgtaatagaaacatcagtccattcctctttcatcttaatcaaggacattcccatggagcccaagattacaaatgtactcagggctgtttattcggtggctccctggtttgcatttacctcatataaagaatgggaaggagaccattgggtaaccctcaagtgtcagaagttgtttctaaagtaactatacatgttttttactaaatctctgcagtgcttataaaatacattgttgcctatttagggagtaacattttctagtttttgtttctggttaaaatgaaatatgggcttatgtcaattcattggaagtcaatgcactaactcaataccaagatgagtttttaaataatgaatattatttaataccacaacagaattatccccaatttccaataagtcctatcattgaaaattcaaatataagtgaagaaaaaattagtagatcaacaatctaaacaaatccctcggttctaagatacaatggattccccatactggaaggactctgaggctttattcccccactatgcatatcttatcattttattattatacacacatccatcctaaactatactaaagcccttttcccatgcatggatggaaatggaagatttttttgtaacttgttctagaagtcttaatatgggctgttgccatgaaggcttgcagaattgagtccattttctagctgcctttattcacatagtgatggggtactaaaagtactgggttgactcagagagtcgctgtcattctgtcattgctgctactctaacactgagcaacactctcccagtggcagatcccctgtatcattccaagaggagcattcatccctttgctctaatgatcaggaatgatgcttattagaaaacaaactgcttgacccaggaacaagtggcttagcttaagtaaacttggctttgctcagatccctgatccttccagctggtctgctctgagtggcttatcccgcatgagcaggagcgtgctggccctgagtactgaactttctgagtaacaatgagacacgttacagaacctatgttcaggttgcgggtgagctgccctctccaaatccagccagagatgcacattcctcggccagtctcagccaacagtaccaaaagtgatttttgagtgtgccagggtaaaggcttccagttcagcctcagttattttagacaatctcgccatctttaatttcttagcttcctgttctaataaatgcacggctttacctttcctgtcagaaataaaccaaggctctaaaagatgatttcccttctgtaactccctagagccacaggttctcattccttttcccattatacttctcacaattcagtttctatgagtttgatcacctgatttttttaacaaaatatttctaacgggaatgggtgggagtgctggtgaaaagagatgaaatgtggttgtatgagccaatcatatttgtgattttttaaaaaaagtttaaaaggaaatatctgttctgaaaccccacttaagcattgtttttatataaaaacaatgataaagatgtgaactgtgaaataaatataccatattagctacccacc

SEQ ID NO: 120 - protein, 440 aa, Homo sapiens
MNYSLHLAFVCLSLFTERMCIQGSQFNVEVGRSDKLSLPGFENLTAGYNKFLRPNFGGEPVQIALTLDIASISSISESNMDYTATIYLRQRWMDQRLVFEGNKSFTLDARLVEFLWVPDTYIVESKKSFLHEVTVGNRLIRLFSNGTVLYALRITTTVACNMDLSKYPMDTQTCKLQLESWGYDGNDVEFTWLRGNDSVRGLEHLRLAQYTIERYFTLVTRSQQETGNYTRLVLQFELRRNVLYFILETYVPSTFLVVLSWVSFWISLDSVPARTCIGVTTVLSMTTLMIGSRTSLPNTNCFIKAIDVYLGICFSFVFGALLEYAVAHYSSLQQMAAKDRGTTKEVEEVSITNIINSSISSFKRKISFASIEISSDNVDYSDLTMKTSDKFKFVFREKMGRIVDYFTIQNPSNVDHYSKLLFPLIFMLANVFYWAYYMYF

SEQ ID NO: 121 - DNA, 6158 bp, Homo sapiens
  modified_base (1)..(6158): n = g, a, c or t
aaccatcaaatttagaagaaaaagccctttgactttttccccctctccctccccaatggctgtgtagcaaacatccctggcgataccttggaaaggacgaagttggtctgcagtcgcaatttcgtgggttgagttcacagttgtgagtgcggggctcggagatggagccgtggtcctctaggtggaaaacgaaacggtggctctgggatttcaccgtaacaaccctcgcattgaccttcctcttccaagctagagaggtcagaggagctgctccagttgatgtactaaaagcactagattttcacaattctccagagggaatatcaaaaacaacgggattttgcacaaacagaaagaattctaaaggctcagatactgcttacagagtttcaaagcaagcacaactcagtgccccaacaaaacagttatttccaggtggaactttcccagaagacttttcaatactatttacagtaaaaccaaaaaaaggaattcagtctttccttttatctatatataatgagcatggtattcagcaaattggtgttgaggttgggagatcacctgtttttctgtttgaagaccacactggaaaacctgccccagaagactatcccctcttcagaactgttaacatcgctgacgggaagtggcatcgggtagcaatcagcgtggagaagaaaactgtgacaatgattgttgattgtaagaagaaaaccacgaaaccacttgatagaagtgagagagcaattgttgataccaatggaatcacggtttttggaacaaggattttggatgaagaagtttttgagggggacattcagcagtttttgatcacaggtgatcccaaggcagcatatgactactgtgagcattatagtccagactgtgactcttcagcacccaaggctgctcaagctcaggaacctcagatagatgagtatgcaccagaggatataatcgaatatgactatgagtatggggaagcagagtataaagaggctgaaagtgtaacagagggacccactgtaactgaggagacaatagcacagacggaggcaaacatcgttgatgattttcaagaatacaactatggaacaatggaaagttaccagacagaagctcctaggcatgtttctgggacaaatgagccaaatccagttgaagaaatatttactgaagaatatctaacgggagaggattatgattcccagaggaaaaattctgaggatacactatatgaaaacaaagaaatagacggcagggattctgatcttctggtagatggagatttaggcgaatatgatttttatgaatataaagaatatgaagataaaccaacaagcccccctaatgaagaatttggtccaggtgtaccagcagaaactgatattacagaaacaagcataaatggccatggtgcatatggagagaaaggacagaaaggagaaccagcagtggttgagcctggtatgcttgtcgaaggaccaccaggaccagcaggacctgcaggtattatgggtcctccaggtctacaaggccccactggaccccctggtgaccctggcgataggggccccccaggacgtcctggcttaccaggggctgatggtctacctggtcctcctggtactatgttgatgttaccgttccgttatggtggtgatggttccaaaggaccaaccatctctgctcaggaagctcaggctcaagctattcttcagcaggctcggattgctctgagaggcccacctggcccaatgggtctaactggaagaccaggtcctgtgggggggcctggttcatctggggccaaaggtgagagtggtgatccaggtcctcagggccctcgaggcgtccagggtccccctggtccaacgggaaaacctggaaaaaggggtcgtccaggtgcagatggaggaagaggaatgccaggagaacctggggcaaagggagatcgagggtttgatggacttccgggtctgccaggtgacaaaggtcacaggggtgaacgaggtcctcaaggtcctccaggtcctcctggtgatgatggaatgaggggagaagatggagaaattggaccaagaggtcttccaggtgaagctggcccacgaggtttgctgggtccaaggggaactccaggagctccagggcagcctggtatggcaggtgtagatggccccccaggaccaaaagggaacatgggtccccaaggggagcctgggcctccaggtcaacaagggaatccaggacctcagggtcttcctggtccacaaggtccaattggtcctcctggtgaaaaaggaccacaaggaaaaccaggacttgctggacttcctggtgctgatgggcctcctggtcatcctgggaaagaaggccagtctggagaaaagggggctctgggtccccctggtccacaaggtcctattggatnnccgggcccccggggagtaaagggagcagatggtgtcagaggtctcaagggatctaaaggtgaaaagggtgaagatggttttccaggattcaaaggtgacatgggtctaaaaggtgacagaggagaagttggtcaaattggcccaagagggnaagatggccctgaaggacccaaaggtcgagcaggcccaactggagacccaggtccttcaggtcaagcaggagaaaagggaaaacttggagttccaggattaccaggatatccaggaagacaaggtccaaagggttccactggattccctgggtttccaggtgccaatggagagaaaggtgcacggggagtagctggcaaaccaggccctcggggtcagcgtggtccaacgggtcctcgaggttcaagaggtgcaagaggtcccactgggaaacctgggccaaagggcacttcaggtggcgatggccctcctggccctccaggtgaaagaggtcctcaaggacctcagggtccagttggattccctggaccaaaaggccctcctggaccaccaggaaggatgggctgcccaggacaccctgggcaacgtggggagactggatttcaaggcaagaccggccctcctgggccagggggagtggttggaccacagggaccaaccggtgagactggtccaataggggaacgtgggtatcctggtcctcctggccctcctggtgagcaaggtcttcctggtgctgcaggaaaagaaggtgcaaagggtgatccaggtcctcaaggtatctcagggaaagatggaccagcaggattacgtggtttcccaggggaaagaggtcttcctggagctcagggtgcacctggactgaaaggaggggaaggtccccagggcccaccaggtccagttggctcaccaggagaacgtgggtcagcaggtacagctggcccaattggtttacgagggcgcccgggacctcagggtcctcctggtccagctggagagaaaggtgctcctggagaaaaaggtccccaagggcctgcagggagagatggagttcaaggtcctgttggtctcccagggccagctggtcctgccggctcccctggggaagacggagacaagggtgaaattggtgagccgggacaaaaaggcagcaagggtggcaagggagaaaatggccctcccggtcccccaggtcttcaaggaccagttggtgcccctggaattgctggaggtgatggtgaaccaggtcctagaggacagcaggggatgtttgggcaaaaaggtgatgagggtgccagaggcttccctggacctcctggtccaataggtcttcagggtctgccaggcccacctggtgaaaaaggtgaaaatggggatgttggtccatgggggccacctggtcctccaggcccaagaggccctcaaggtcccaatggagctgatggaccacaaggacccccaggttctgttggttcagttggtggtgttggagaaaagggtgaacctggagaagcaggaaacccagggcctcctggggaagcaggtgtaggcggtcccaaaggagaaagaggagagaaaggggaagctggtccacctggagctgctggacctccaggtgccaaggggccgccaggtgatgatggccctaagggtaacccgggtcctgttggttttcctggagatcctggtcctcctggggaacttggccctgcaggtcaagatggtgttggtggtgacaagggtgaagatggagatcctggtcaaccgggtcctcctggcccatctggtgaggctggcccaccaggtcctcctggaaaacgaggtcctcctggagctgcaggtgcagagggaagacaaggtgaaaaaggtgctaagggggaagcaggtgcagaaggtcctcctggaaaaaccggcccagtcggtcctcagggacctgcaggaaagcctggtccagaaggtcttcggggcatccctggtcctgtgggagaacaaggtctccctggagctgcaggccaagatggaccacctggtcctatgggacctcctggcttacctggtctcaaaggtgaccctggctccaagggtgaaaagggacatcctggtttaattggcctgattggtcctccaggagaacaaggggaaaaaggtgaccgagggctccctggaactcaaggatctccaggagcaaaaggggatgggggaattcctggtcctgctggtcccttaggtccacctggtcctccaggcttaccaggtcctcaaggcccaaagggtaacaaaggctctactggacccgctggccagaaaggtgacagtggtcttccagggcctcctgggcctccaggtccacctggtgaagtcattcagcctttaccaatcttgtcctccaaaaaaacgagaagacatactgaaggcatgcaagcagatgcagatgataatattcttgattactcggatggaatggaagaaatatttggttccctcaattccctgaaacaagacatcgagcatatgaaatttccaatgggtactcagaccaatccagcccgaacttgtaaagacctgcaactcagccatcctgacttcccagatggtgaatattggattgatcctaaccaaggttgctcaggagattccttcaaagtttactgtaatttcacatctggtggtgagacttgcatttatccagacaaaaaatctgagggagtaagaatttcatcatggccaaaggagaaaccaggaagttggtttagtgaatttaagaggggaaaactgctttcatacttagatgttgaaggaaattccatcaatatggtgcaaatgacattcctgaaacttctgactgcctctgctcggcaaaatttcacctaccactgtcatcagtcagcagcctggtatgatgtgtcatcaggaagttatgacaaagcacttcgcttcctgggatcaaatgatgaggagatgtcctatgacaataatccttttatcaaaacactgtatgatggttgtacgtccagaaaaggctatgaaaaaactgtcattgaaatcaatacaccaaaaattgatcaagtacctattgttgatgtcatgatcagtgactttggtgatcagaatcagaagttcggatttgaagttggtcctgtttgttttcttggctaagattaagacaaagaacatatcaaatcaacagaaaatgtaccttggtgccaccaacccattttgtgccacatgcaagttttgaataaggatgtatggaaaacaacgctgcatatacaggtaccatttaggaaataccgatgcctttgtgggggcagaatcacagacaaaagctttgaaaatcataaagatataagttggtgtggctaagatggaaacagggctgattcttgattcccaattctcaactctccttttcctatttgaatttctttggtgctgtagaaaacaaaaaaagaaaaatatatattcataaaaaatatggtgctcattctcatccatccaggatgtactaaaacagtgtgtttaataaattgtaattattttgtgtacagttctatactgttatctgtgtccatttccaaaacttgcacgtgtccctgaattccgctgactctaatttatgaggatgccgaactctgatggcaataatatatgtattatgaaaatgaagttatgatttccgatgaccctaagtccctttctttggttaatgatgaaattcctttgtgtgtgttt

SEQ ID NO: 122 - protein, 1806 aa, Homo sapiens
  MOD_RES (1)..(1806): Xaa = any amino acid
MEPWSSRWKTKRWLWDFTVTTLALTFLFQAREVRGAAPVDVLKALDFHNSPEGISKTTGFCTNRKNSKGSDTAYRVSKQAQLSAPTKQLFPGGTFPEDFSILFTVKPKKGIQSFLLSIYNEHGIQQIGVEVGRSPVFLFEDHTGKPAPEDYPLFRTVNIADGKWHRVAISVEKKTVTMIVDCKKKTTKPLDRSERAIVDTNGITVFGTRILDEEVFEGDIQQFLITGDPKAAYDYCEHYSPDCDSSAPKAAQAQEPQIDEYAPEDIIEYDYEYGEAEYKEAESVTEGPTVTEETIAQTEANIVDDFQEYNYGTMESYQTEAPRHVSGTNEPNPVEEIFTEEYLTGEDYDSQRKNSEDTLYENKEIDGRDSDLLVDGDLGEYDFYEYKEYEDKPTSPPNEEFGPGVPAETDITETSINGHGAYGEKGQKGEPAVVEPGMLVEGPPGPAGPAGIMGPPGLQGPTGPPGDPGDRGPPGRPGLPGADGLPGPPGTMLMLPFRYGGDGSKGPTISAQEAQAQAILQQARIALRGPPGPMGLTGRPGPVGGPGSSGAKGESGDPGPQGPRGVQGPPGPTGKPGKRGRPGADGGRGMPGEPGAKGDRGFDGLPGLPGDKGHRGERGPQGPPGPPGDDGMRGEDGEIGPRGLPGEAGPRGLLGPRGTPGAPGQPGMAGVDGPPGPKGNMGPQGEPGPPGQQGNPGPQGLPGPQGPIGPPGEKGPQGKPGLAGLPGADGPPGHPGKEGQSGEKGALGPPGPQGPIGXPGPRGVKGADGVRGLKGSKGEKGEDGFPGFKGDMGLKGDRGEVGQIGPRGXDGPEGPKGRAGPTGDPGPSGQAGEKGKLGVPGLPGYPGRQGPKGSTGFPGFPGANGEKGARGVAGKPGPRGQRGPTGPRGSRGARGPTGKPGPKGTSGGDGPPGPPGERGPQGPQGPVGFPGPKGPPGPPGRMGCPGHPGQRGETGFQGKTGPPGPGGVVGPQGPTGETGPIGERGYPGPPGPPGEQGLPGAAGKEGAKGDPGPQGISGKDGPAGLRGFPGERGLPGAQGAPGLKGGEGPQGPPGPVGSPGERGSAGTAGPIGLRGRPGPQGPPGPAGEKGAPGEKGPQGPAGRDGVQGPVGLPGPAGPAGSPGEDGDKGEIGEPGQKGSKGGKGENGPPGPPGLQGPVGAPGIAGGDGEPGPRGQQGMFGQKGDEGARGFPGPPGPIGLQGLPGPPGEKGENGDVGPWGPPGPPGPRGPQGPNGADGPQGPPGSVGSVGGVGEKGEPGEAGNPGPPGEAGVGGPKGERGEKGEAGPPGAAGPPGAKGPPGDDGPKGNPGPVGFPGDPGPPGELGPAGQDGVGGDKGEDGDPGQPGPPGPSGEAGPPGPPGKRGPPGAAGAEGRQGEKGAKGEAGAEGPPGKTGPVGPQGPAGKPGPEGLRGIPGPVGEQGLPGAAGQDGPPGPMGPPGLPGLKGDPGSKGEKGHPGLIGLIGPPGEQGEKGDRGLPGTQGSPGAKGDGGIPGPAGPLGPPGPPGLPGPQGPKGNKGSTGPAGQKGDSGLPGPPGPPGPPGEVIQPLPILSSKKTRRHTEGMQADADDNILDYSDGMEEIFGSLNSLKQDIEHMKFPMGTQTNPARTCKDLQLSHPDFPDGEYWIDPNQGCSGDSFKVYCNFTSGGETCIYPDKKSEGVRISSWPKEKPGSWFSEFKRGKLLSYLDVEGNSINMVQMTFLKLLTASARQNFTYHCHQSAAWYDVSSGSYDKALRFLGSNDEEMSYDNNPFIKTLYDGCTSRKGYEKTVIEINTPKIDQVPIVDVMISDFGDQNQKFGFEVGPVCFLG

SEQ ID NO: 123 - DNA, 1440 bp, Homo sapiens
gaattccccccccccccccctcacttggtgtgtctatatgtctggcagacattatcagcacattctctgttgttacctgtgattcattttttcttcactctccaggtgaatttcaattgctgaaaatttcccactgaaaatatgcagtaatatattttgtggttcagacatttggggcaaatggttcacattcattttagggttagtggtcatgctgtttatttttctctgctatacaaagttcctcttaggggtctgcctcatgacactaaaaaatgaatagagattctactgtaggttatctcctaggcttgagttcaacatttgtttggatttttgaagaaagtcaaatcaagcaatgctcccaaatgatgtctttgtaaattcataccctctggccctattttttttcatagaccctaactctacctttctgctttaaagcaaagtaaactcggtggcctcttcttctccacccctcaaaatgatagcaatctctgccgtcagcagtgcactcctgttctcccttctctgtgaagcaagtaccgtcgtcctactcaattccactgactcatccccgccaaccaataatttcactgatattgaagcagctctgaaagcacaattagattcagcggatatccccaaagccaggcggaagcgctacatttcgcagaatgacatgatcgccattcttgattatcataatcaagttcggggcaaagtgttcccaccggcagcaaatatggaatatatggtttgggatgaaaatcttgcaaaatcggcagaggcttgggcggctacttgcatttgggaccatggaccttcttacttactgagatttttgggccaaaatctatctgtacgcactggaagatatcgctctattctccagttggtcaagccatggtatgatgaagtgaaagattatgcttttccatatccccaggattgcaaccccagatgtcctatgagatgttttggtcccatgtgcacacattatacgcagatggtttgggccacttccaatcggataggatgcgcaattcatgcttgccaaaacatgaatgtttggggatctgtgtggcgacgtgcagtttacttggtatgcaactatgccccaaagggcaattggattggagaagcaccatataaagtaggggtaccatgttcatcttgtcctccaagttatgggggatcttgtactgacaatctgtgttttccaggagttacgtcaaactacctgtactggtttaaataagtttaccttttcctccaggaaatataatgatttctgggaacatgggcatgtatatatatatatggagagagaattttgcacatattatacatattttgtgctaatcttgttttcctcttagtattcctttgtataaattagtgtttgtctagcatgtttgtttaatcctttgggaattc

SEQ ID NO: 124 - protein, 258 aa, Homo sapiens
MIAISAVSSALLFSLLCEASTVVLLNSTDSSPPTNNFTDIEAALKAQLDSADIPKARRKRYISQNDMIAILDYHNQVRGKVFPPAANMEYMVWDENLAKSAEAWAATCIWDHGPSYLLRFLGQNLSVRTGRYRSILQLVKPWYDEVKDYAFPYPQDCNPRCPMRCFGPMCTHYTQMVWATSNRIGCAIHACQNMNVWGSVWRRAVYLVCNYAPKGNWIGEAPYKVGVPCSSCPPSYGGSCTDNLCFPGVTSNYLYWFK

SEQ ID NO: 125 - DNA, 3171 bp, Homo sapiens
gcggaacaccggcccgccgtcgcggcagctgcttcacccctctctctgcagccatggggctccctcgtggacctctcgcgtctctcctccttctccaggtttgctggctgcagtgcgcggcctccgagccgtgccgggcggtcttcagggaggctgaagtgaccttggaggcgggaggcgcggagcaggagcccggccaggcgctggggaaagtattcatgggctgccctgggcaagagccagctctgtttagcactgataatgatgacttcactgtgcggaatggcgagacagtccaggaaagaaggtcactgaaggaaaggaatccattgaagatcttcccatccaaacgtatcttacgaagacacaagagagattgggtggttgctccaatatctgtccctgaaaatggcaagggtcccttcccccagagactgaatcagctcaagtctaataaagatagagacaccaagattttctacagcatcacggggccgggggcagacagcccccctgagggtgtcttcgctgtagagaaggagacaggctggttgttgttgaataagccactggaccgggaggagattgccaagtatgagctctttggccacgctgtgtcagagaatggtgcctcagtggaggaccccatgaacatctccatcatcgtgaccgaccagaatgaccacaagcccaagtttacccaggacaccttccgagggagtgtcttagagggagtcctaccaggtacttctgtgatgcaggtgacagccacagatgaggatgatgccatctacacctacaatggggtggttgcttactccatccatagccaagaaccaaaggacccacacgacctcatgttcacaattcaccggagcacaggcaccatcagcgtcatctccagtggcctggaccgggaaaaagtccctgagtacacactgaccatccaggccacagacatggatggggacggctccaccaccacggcagtggcagtagtggagatccttgatgccaatgacaatgctcccatgtttgacccccagaagtacgaggcccatgtgcctgagaatgcagtgggccatgaggtgcagaggctgacggtcactgatctggacgcccccaactcaccagcgtggcgtgccacctaccttatcatgggcggtgacgacggggaccattttaccatcaccacccaccctgagagcaaccagggcatcctgacaaccaggaagggtttggattttgaggccaaaaaccagcacaccctgtacgttgaagtgaccaacgaggccccttttgtgctgaagctcccaacctccacagccaccatagtggtccacgtggaggatgtgaatgaggcacctgtgtttgtcccaccctccaaagtcgttgaggtccaggagggcatccccactggggagcctgtgtgtgtctacactgcagaagaccctgacaaggagaatcaaaagatcagctaccgcatcctgagagacccagcagggtggctagccatggacccagacagtgggcaggtcacagctgtgggcaccctcgaccgtgaggatgagcagtttgtgaggaacaacatctatgaagtcatggtcttggccatggacaatggaagccctcccaccactggcacgggaacccttctgctaacactgattgatgtcaacgaccatggcccagtccctgagccccgtcagatcaccatctgcaaccaaagccctgtgcgccacgtgctgaacatcacggacaaggacctgtctccccacacctcccctttccaggcccagctcacagatgactcagacatctactggacggcagaggtcaacgaggaaggtgacacagtggtcttgtccctgaagaagttcctgaagcaggatacatatgacgtgcacctttctctgtctgaccatggcaacaaagagcagctgacggtgatcagggccactgtgtgcgactgccatggccatgtcgaaacctgccctggaccctggaaaggaggtttcatcctccctgtgctgggggctgtcctggctctgctgttcctcctgctggtgctgcttttgttggtgagaaagaagcggaagatcaaggagcccctcctactcccagaagatgacacccgtgacaacgtcttctactatggcgaagaggggggtggcgaagaggaccaggactatgacatcacccagctccaccgaggtctggaggccaggccggaggtggttctccgcaatgacgtggcaccaaccatcatcccgacacccatgtaccgtcctaggccagccaacccagatgaaatcggcaactttataattgagaacctgaaggcggctaacacagaccccacagccccgccctacgacaccctcttggtgttcgactatgagggcagcggctccgacgccgcgtccctgagctccctcacctcctccgcctccgaccaagaccaagattacgattatctgaacgagtggggcagccgcttcaagaagctggcagacatgtacggtggcggggaggacgactaggcggcctgcctgcagggctggggaccaaacgtcaggccacagagcatctccaaggggtctcagttcccccttcagctgaggacttcggagcttgtcaggaagtggccgtagcaacttggcggagacaggctatgagtctgacgttagagtggttgcttccttagcctttcaggatggaggaatgtgggcagtttgacttcagcactgaaaacctctccacctgggccagggttgcctcagaggccaagtttccagaagcctcttacctgccgtaaaatgctcaaccctgtgtcctgggcctgggcctgctgtgactgacctacagtggactttctctctggaatggaaccttcttaggcctcctggtgcaacttaatttttttttttaatgctatcttcaaaacgttagagaaagttcttcaaaagtgcagcccagagctgctgggcccactggccgtcctgcatttctggtttccagaccccaatgcctcccattcggatggatctctgcgtttttatactgagtgtgcctaggttgccccttattttttattttccctgttgcgttgctatagatgaagggtgaggacaatcgtgtatatgtactagaacttttttattaaagaaa

SEQ ID NO: 126 - protein, 829 aa, Homo sapiens [sequence omitted]

SEQ ID NO: 127 - DNA, 1189 bp, Homo sapiens
cctgctggggccgtccagtcccccagacctcacaggctcagtcgcggatctgcagtgtcatgcctgggagccctcggcccgcgccaagctgggtgctgttgctgcggctgctggcgttgctgcggcccccggggctgggtgaggcatgcagctgcgccccggcgcaccctcagcagcacatctgccactcggcacttgtgattcgggccaaaatctccagtgagaaggtagttccggccagtgcagaccctgctgacactgaaaaaatgctccggtatgaaatcaaacagataaagatgttcaaagggtttgagaaagtcaaggatgttcagtatatctatacgccttttgactcttccctctgtggtgtgaaactagaagccaacagccagaagcagtatctcttgactggtcaggtcctcagtgatggaaaagtcttcatccatctgtgcaactacatcgagccctgggaggacctgtccttggtgcagagggaaagtctgaatcatcactaccatctgaactgtggctgccaaatcaccacctgctacacagtaccctgtaccatctcggcccctaacgagtgcctctggacagactggctgttggaacgaaagctctatggttaccaggctcagcattatgtctgtatgaagcatgttgacggcacctgcagctggtaccggggccacctgcctctcaggaaggagtttgttgacatcgttcagccctagtagggaccagtgaccatcacatcccttcaagagtcctgaagatcaagccagttctccttccctgcagagctttggccattaccacctgacctcttgctgccagctaataagaagtgccaagtggacagtctggccactgtcaaggcagggaaggggccatgacttttctgccctgccctcagcctgttgccctgcctcccaaaccccattagtctagccttgtagctgttactgcaagtgtttcttctggcttagtctgttttctaaagccaggactattccctttcctccccaggaatatgtgttttcctttgtcttaatcgatctggtaggggagaaatggcgaatgtcatacacatgagatggtatatccttgcgatgtacagaatcagaaggtggtttgacagcatcataaacaggctgactggcaggaatgaaaaaaaaaaaaaaaaaaa

SEQ ID NO: 128 - protein, 224 aa, Homo sapiens
MPGSPRPAPSWVLLLRLLALLRPPGLGEACSCAPAHPQQHICHSALVIRAKISSEKVVPASADPADTEKMLRYEIKQIKMFKGFEKVKDVQYIYTPFDSSLCGVKLEANSQKQYLLTGQVLSDGKVFIHLCNYIEPWEDLSLVQRESLNHHYHLNCGCQITTCYTVPCTISAPNECLWTDWLLERKLYGYQAQHYVCMKHVDGTCSWYRGHLPLRKEFVDIVQP

SEQ ID NO: 129 - DNA, 1909 bp, Homo sapiens
  modified_base (1891): n = g, a, c or t
ccacgcgtccgcaatgaagccgagtgaatgggggctgaatgtgcgagtccatagctgaagaggagcgccagatggtggaggaatacacttatttatgaaactgtcttgagttcttcttgaattgccagttttcagcctcctcatgcctccgtctcctttagacgacagggtagtagtggcactatctaggcccgtccgacctcaggatctcaacctttgtttagactctagttaccttggctctgccaacccaggcagtaacagccaccctcctgtcatcgccaccaccgttgtgtccctcaaggctgcgaatctgacgtatatgccctcatccagcggctctgcccgctcgctgaattgtggatgcagcagtgccagctgctgcactgtggcaacctacgacaaggacaatcaggcccaaacccaagccattgccgctggcaccaccaccactgccatcggaacctctaccacctgccctgctaaccagatggtcaacaataatgagaatacaggctctctaagtccatcaagtggggtgggcagccctgtgtcagggacccccaagcagctagccagcatcaaaataatctaccccaatgacttggcaaagaagatgaccaaatgcagcaagagtcacctgccgagtcagggccctgtcatcattgactgcaggcccttcatggagtacaacaagagtcacatccaaggagctgtccacattaactgtgccgataagatcagccggcggagactgcagcagggcaagatcactgtcctagacttgatttcctgtagggaaggcaaggactctttcaagaggatcttttccaaagaaattatagtttatgatgagaataccaatgaaccaagccgagtgatgccctcccagccacttcacatagtcctcgagtccctgaagagagaaggcaaagaacctctggtgttgaaaggtggacttagtagttttaagcagaaccatgaaaacctctgtgacaactccctccagctccaagagtgccgggaggtggggggcggcgcatccgcggcctcgagcttgctacctcagcccatccccaccacccctgacatcgagaacgctgagctcacccccatcttgcccttcctgttccttggcaatgagcaggatgctcaggacctggacaccatgcagcggctgaacatcggctacgtcatcaacgtcaccactcatcttcccctctaccactatgagaaaggcctgttcaactacaagcggctgccagccactgacagcaacaagcagaacctgcggcagtactttgaagaggcttttgagttcattgaggaagctcaccagtgtgggaaggggcttctcatccactgccaggctggggtgtcccgctccgccaccatcgtcatcgcttacttgatgaagcacactcggatgaccatgactgatgcttataaatttgtcaaaggcaaacgaccaattatctccccaaaccttaacttcatggggcagttgctagagttcgaggaagacctaaacaacggtgtgacaccgagaatccttacaccaaagctgatgggcgtggagacggttgtgtgacaatggtctggatggaaaggattgctgctctccattaggagacaatgaggaaggaggatggattctggttttttttctttcttttttttttgtagttgggagtaaagtttgtgaatggaaacaaacttggttaaacactttatttttaacaagtgtaagaagactatacttttgatgccattgagattcaccttccacaaactggccaaattaaggaggttaaagaagtaattttttttaagcccaaccattaaaaatttaatacaacttggtttctccccctttttcctttaaagctantttgtaaaagtttatgag

SEQ ID NO: 130 - protein, 482 aa, Homo sapiens
MPPSPLDDRVVVALSRPVRPQDLNLCLDSSYLGSANPGSNSHPPVIATTVVSLKAANLTYMPSSSGSARSLNCGCSSASCCTVATYDKDNQAQTQAIAAGTTTTAIGTSTTCPANQMVNNNENTGSLSPSSGVGSPVSGTPKQLASIKIIYPNDLAKKMTKCSKSHLPSQGPVIIDCRPFMEYNKSHIQGAVHINCADKISRRRLQQGKITVLDLISCREGKDSFKRIFSKEIIVYDENTNEPSRVMPSQPLHIVLESLKREGKEPLVLKGGLSSFKQNHENLCDNSLQLQECREVGGGASAASSLLPQPIPTTPDIENAELTPILPFLFLGNEQDAQDLDTMQRLNIGYVINVTTHLPLYHYEKGLFNYKRLPATDSNKQNLRQYFEEAFEFIEEAHQCGKGLLIHCQAGVSRSATIVIAYLMKHTRMTMTDAYKFVKGKRPIISPNLNFMGQLLEFEEDLNNGVTPRILTPKLMGVETVV

SEQ ID NO: 131 - DNA, 1493 bp, Homo sapiens
ttcctttcatgttcagcatttctactccttccaagaagagcagcaaagctgaagtagcagcaacagcaccagcagcaacagcaaaaaacaaacatgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttttaaaaatatcaaaacatatgaagtcctggaaaagggcatctgaaaaacctagaacaagtttaactgtgactactgaaatgacaagaattctacagtaggaaactgagacttttctatggttttgtgactttcaacttttgtacagttatgtgaaggatgaaaggtgggtgaaaggaccaaaaacagaaatacagtcttcctgaatgaatgacaatcagaattccactgcccaaaggagtccagcaattaaatggatttctaggaaaagctaccttaagaaaggctggttaccatcggagtttacaaagtgctttcacgttcttacttgttgtattatacattcatgcatttctaggctagagaaccttctagatttgatgcttacaactattctgttgtgactatgagaacatttctgtctctagaagttatctgtctgtattgatctttatgctatattactatctgtggttacagtggagacattgacattattactggagtcaagcccttataagtcaaaagcatctatgtgtcgtaaagcattcctcaaacattttttcatgcaaatacacacttctttccccaaatatcatgtagcacatcaatatgtagggaaacattcttatgcatcatttggtttgttttataaccaattcattaaatgtaattcataaaatgtactatgaaaaaaattatacgctatgggatactggcaacagtgcacatatttcataaccaaattagcagcaccggtcttaatttgatgtttttcaacttttattcattgagatgttttgaagcaattaggatatgtgtgtttactgtactttttgttttgatccgtttgtataaatgatagcaatatcttggacacatttgaaatacaaaatgtttttgtctaccaaagaaaaatgttgaaaaataagcaaatgtatacctagcaatcacttttactttttgtaattctgtctcttagaaaaatacataatctaatcaatttctttgttcatgcctatatactgtaaaatttaggtatactcaagactagtttaaagaatcaaagtcatttttttctctaataaactaccacaacctttcttttttaaaaaaaaaaa

SEQ ID NO: 132 - protein, 94 aa, Homo sapiens
MSVKGMAIALAVILCATVVQGFPMFKRGRCLCIGPGVKAVKVADIEKASIMYPSNNCDKIEVIITLKENKGQRCLNPKSKQARLIIKKVERKNF

SEQ ID NO: 133 - DNA, 1521 bp, Homo sapiens
ctggcgcgggcgggagctgcggcggatacccttgcgtgctgtggagaccctactctcttcgctgagaacggccgctagcggggactgaaggccgggagcccactcccgacccggggctagcgtgcgtccctagagtcgagcggggcaagggagccagtggccgccgacgggggaccgggaaacttttctgggctcctggagagccctgtagccgcgctccatgctccggcagcggcccgaaacccagccccgccgctgacggagcccgccgctccgggcagggcccatgccctgcgcgctccgggggtcgtagctgccgccgagccggggctccggaagccggcgggggcgccgcggccgtgcggggcgtcaatggatcgccactccagctacatcttcatctggctgcagctggagctctgcgccatggccgtgctgctcaccaaaggtgaaattcgatgctactgtgatgctgcccactgtgtagccactggttatatgtgtaaatctgagctcagcgcctgcttctctagacttcttgatcctcagaactcaaattccccactcacccatggctgcctggactctcttgcaagcacgacagacatctgccaagccaaacaggcccgaaaccactctggcaccaccatacccacattggaatgctgtcatgaagacatgtgcaattacagagggctgcacgatgttctctctcctcccaggggtgaggcctcaggacaaggaaacaggtatcagcatgatggtagcagaaaccttatcaccaaggtgcaggagctgacttcttccaaagagttgtggttccgggcagcggtcattgccgtgcccattgctggagggctgattttagtgttgcttattatgttggccctgaggatgcttcgaagtgaaaataagaggctgcaggatcagcggcaacagatgctctcccgtttgcactacagctttcacggacaccattccaaaaaggggcaggttgcaaagttagacttggaatgcatggtgccggtcagtgggcacgagaactgctgtctgacctgtgataaaatgagacaagcagacctcagcaacgataagatcctctcgcttgttcactggggcatgtacagtgggcacgggaagctggaattcgtatgacggagtcttatctgaactacacttactgaacagcttgaaggccttttgagttctgctggacaggagcactttatctgaagacaaactcatttaatcatctttgagagacaaaatgacctctgcaaacagaatcttggatatttcttctgaaggattatttgcacagacttaaatacagttaaatgtgttatttgcttttaaaattataaaaagcaaagagaagactttgtacacactgtcaccagggttatttgcatccaagggagctggaattgagtacctaaataaacaaaaatgtgccctatgtaagcttctacatcttgatttattgtaaagatttaaaagaaatatatatattttgtctga

SEQ ID NO: 134 - protein, 260 aa, Homo sapiens
MDRHSSYIFIWLQLELCAMAVLLTKGEIRCYCDAAHCVATGYMCKSELSACFSRLLDPQNSNSPLTHGCLDSLASTTDICQAKQARNHSGTTIPTLECCHEDMCNYRGLHDVLSPPRGEASGQGNRYQHDGSRNLITKVQELTSSKELWFRAAVIAVPIAGGLILVLLIMLALRMLRSENKRLQDQRQQMLSRLHYSFHGHHSKKGQVAKLDLECMVPVSGHENCCLTCDKMRQADLSNDKILSLVHWGMYSGHGKLEFV

SEQ ID NO: 135 - DNA, 2539 bp, Homo sapiens
cgggacgacgccccctcctgcggcgtggactccgtcagtggcccaccaagaaggaggaggaatatggaatccaagggggccagttcctgccgtctgctcttctgcctcttgatctccgccaccgtcttcaggccaggccttggatggtatactgtaaattcagcatatggagataccattatcataccttgccgacttgacgtacctcagaatctcatgtttggcaaatggaaatatgaaaagcccgatggctccccagtatttattgccttcagatcctctacaaagaaaagtgtgcagtacgacgatgtaccagaatacaaagacagattgaacctctcagaaaactacactttgtctatcagtaatgcaaggatcagtgatgaaaagagatttgtgtgcatgctagtaactgaggacaacgtgtttgaggcacctacaatagtcaaggtgttcaagcaaccatctaaacctgaaattgtaagcaaagcactgtttctcgaaacagagcagctaaaaaagttgggtgactgcatttcagaagacagttatccagatggcaatatcacatggtacaggaatggaaaagtgctacatccccttgaaggagcggtggtcataatttttaaaaaggaaatggacccagtgactcagctctataccatgacttccaccctggagtacaagacaaccaaggctgacatacaaatgccattcacctgctcggtgacatattatggaccatctggccagaaaacaattcattctgaacaggcagtatttgatatttactatcctacagagcaggtgacaatacaagtgctgccaccaaaaaatgccatcaaagaaggggataacatcactcttaaatgcttagggaatggcaaccctcccccagaggaatttttgttttacttaccaggacagcccgaaggaataagaagctcaaatacttacacactgatggatgtgaggcgcaatgcaacaggagactacaagtgttccctgatagacaaaaaaagcatgattgcttcaacagccatcacagttcactatttggatttgtccttaaacccaagtggagaagtgactagacagattggtgatgccctacccgtgtcatgcacaatatctgctagcaggaatgcaactgtggtatggatgaaagataacatcaggcttcgatctagcccgtcattttctagtcttcattatcaggatgctggaaactatgtctgcgaaactgctctgcaggaggttgaaggactaaagaaaagagagtcattgactctcattgtagaaggcaaacctcaaataaaaatgacaaagaaaactgatcccagtggactatctaaaacaataatctgccatgtggaaggttttccaaagccagccattcagtggacaattactggcagtggaagcgtcataaaccaaacagaggaatctccttatattaatggcaggtattatagtaaaattatcatttcccctgaagagaatgttacattaacttgcacagcagaaaaccaactggagagaacagtaaactccttgaatgtctctgctataagtattccagaacacgatgaggcagacgagataagtgatgaaaacagagaaaaggtgaatgaccaggcaaaactaattgtgggaatcgttgttggtctcctccttgctgcccttgttgctggtgtcgtctactggctgtacatgaagaagtcaaagactgcatcaaaacatgtaaacaaggacctcggtaatatggaagaaaacaaaaagttagaagaaaacaatcacaaaactgaagcctaagagagaaactgtcctagttgtccagagataaaaatcatatagaccaattgaagcatgaacgtggattgtatttaagacataaacaaagacattgacagcaattcatggttcaagtattaagcagttcattctaccaagctgtcacaggttttcagagaattatctcaagtaaaacaaatgaaatttaattacaaacaataagaacaagttttggcagccatgataataggtcatatgttgtgtttggttcaattttttttccgtaaatgtctgcactgaggatttctttttggtttgccttttatgtaaattttttacgtagctatttttatacactgtaagctttgttctgggagttgctgttaatctgatgtataatgtaatgtttttatttcaattgtttatatggataatctgagcaggtacatttctgattctgattgctatcagcaatgccccaaactttctcataagcacctaaaacccaaaggtggcagcttgtgaagattggggacactcatattgccctaattaaaaactgtgatttttatcacaagggaggggaggccgagagtcagactgatagacaccataggagccgactctttgatatgccaccagcgaactctcagaaataaatcacagatgcatatagacacacatacataatggtactcccaaactgacaattttacctattctgaaaaagacataaaacagaatt

SEQ ID NO: 136 - protein, 583 aa, Homo sapiens
MESKGASSCRLLFCLLISATVFRPGLGWYTVNSAYGDTIIIPCRLDVPQNLMFGKWKYEKPDGSPVFIAFRSSTKKSVQYDDVPEYKDRLNLSENYTLSISNARISDEKRFVCMLVTEDNVFEAPTIVKVFKQPSKPEIVSKALFLETEQLKKLGDCISEDSYPDGNITWYRNGKVLHPLEGAVVIIFKKEMDPVTQLYTMTSTLEYKTTKADIQMPFTCSVTYYGPSGQKTIHSEQAVFDIYYPTEQVTIQVLPPKNAIKEGDNITLKCLGNGNPPPEEFLFYLPGQPEGIRSSNTYTLMDVRRNATGDYKCSLIDKKSMIASTAITVHYLDLSLNPSGEVTRQIGDALPVSCTISASRNATVVWMKDNIRLRSSPSFSSLHYQDAGNYVCETALQEVEGLKKRESLTLIVEGKPQIKMTKKTDPSGLSKTIICHVEGFPKPAIQWTITGSGSVINQTEESPYINGRYYSKIIISPEENVTLTCTAENQLERTVNSLNVSAISIPEHDEADEISDENREKVNDQAKLIVGIVVGLLLAALVAGVVYWLYMKKSKTASKHVNKDLGNMEENKKLEENNHKTEA

SEQ ID NO: 137 - DNA, 1119 bp, Homo sapiens
atgaaccgcagccaccggcacggggcgggcagcggctgcctgggcactatggaggtgaagagcaagtttggagctgaatttcgtcggttttcgctggaaagatcaaaacctggaaaatttgaggagttttatggattactacaacatgttcataagatccccaatgttgacgttttggtaggctatgcagacatccatggagacttactacctataaataatgatgataattatcacaaagctgtttcaacggccaatccactgcttaggatatttatacaaaagaaggaagaagcagactacagtgcctttggtacagacacgctaataaagaagaagaatgttttaaccaacgtattgcgtcctgacaaccatagaaaaaagccacatatagtcattagtatgccccaagactttagacctgtgtcttctattatagacgtggatattctcccagaaacgcatcgtagggtacgtctttacaaatacggcacggagaaacccctaggattctacatccgggatggctccagtgtcagggtaacaccacatggcttagaaaaggttccagggatctttatatccaggcttgtcccaggaggtctggctcaaagtacaggactattagctgttaatgatgaagttttagaagttaatggcatagaagtttcagggaagagccttgatcaagtaacagacatgatgattgcaaatagccgtaacctcatcataacagtgagaccggcaaaccagaggaataatgttgtgaggaacagtcggacttctggcagttccggtcagtctactgataacagccttcttggctacccacagcagattgaaccaagctttgagccagaggatgaagacagcgaagaagatgacattatcattgaagacaatggagtgccacagcagattccaaaagctgttcctaatactgagagcctggagtcattaacacagatagagctaagctttgagtctggacagaatggctttattccctctaatgaagtgagcttagcagccatagcaagcagctcaaacacggaatttgaaacacatgctccagatcaaaaactcttagaagaagatggaacaatcataacattatga

SEQ ID NO: 138 - protein, 372 aa, Homo sapiens
MNRSHRHGAGSGCLGTMEVKSKFGAEFRRFSLERSKPGKFEEFYGLLQHVHKIPNVDVLVGYADIHGDLLPINNDDNYHKAVSTANPLLRIFIQKKEEADYSAFGTDTLIKKKNVLTNVLRPDNHRKKPHIVISMPQDFRPVSSIIDVDILPETHRRVRLYKYGTEKPLGFYIRDGSSVRVTPHGLEKVPGIFISRLVPGGLAQSTGLLAVNDEVLEVNGIEVSGKSLDQVTDMMIANSRNLIITVRPANQRNNVVRNSRTSGSSGQSTDNSLLGYPQQIEPSFEPEDEDSEEDDIIIEDNGVPQQIPKAVPNTESLESLTQIELSFESGQNGFIPSNEVSLAAIASSSNTEFETHAPDQKLLEEDGTIITL

SEQ ID NO: 139 - protein, 6 aa, Artificial Sequence
  Description of Artificial SequenceHIS6 epitope tag
HHHHHH

SEQ ID NO: 140 - DNA, 24 bp, Artificial Sequence
  Description of Artificial SequenceT7-T24 oligo
tttttttttttttttttttttttt

SEQ ID NO: 141 - protein, 4 aa, Artificial Sequence
  Description of Artificial Sequenceendoplasmic reticulum retention sequence
KDEL

(19) United States
(12) Patent Application Publication
(10) Pub. No.: US 2004/0029114 A1
(43) Pub. Date: Feb. 12, 2004

(54) Methods of diagnosis of breast cancer, compositions and methods of screening for modulators of breast cancer

(75) Inventors: David H. Mack, Menlo Park, CA (US); Kurt C. Gish, San Francisco, CA (US); Daniel Afar, Brisbane, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Eos Technology, Inc., South San Francisco, CA (US)

(21) Appl. No.: 10/058,270

(22) Filed: Jan. 24, 2002

Related U.S. Application Data
(60) Provisional application No. 60/263,965, filed on Jan. 24, 2001.
(60) Provisional application No. 60/265,928, filed on Feb. 2, 2001.
(60) Provisional application No. 60/282,698, filed on Apr. 9, 2001.
(60) Provisional application No. 60/288,590, filed on May 4, 2001.
(60) Provisional application No. 60/294,443, filed on May 29, 2001.

(51) Int. Cl. C12Q 1/68; C07H 21/04; C07K 14/72; C12P 21/02; C12N 5/06

(52) U.S. Cl. 435/6; 435/69.1; 435/320.1; 435/325; 530/350; 536/23.5

(57) ABSTRACT

Described herein are genes whose expression are up-regulated or down-regulated in breast cancer. Related methods and compositions that can be used for diagnosis and treatment of breast cancer are disclosed. Also described herein are methods that can be used to identify modulators of breast cancer.

### CROSS-REFERENCES TO RELATED APPLICATIONS

This application claims priority to U.S. S No. 60/263,965, filed Jan. 24, 2001; U.S. S No. 60/265,928, filed Feb. 2, 2001; U.S. Ser. No. 09/829,472 filed Apr. 9, 2001; U.S. S No. 60/282,698, filed Apr. 9, 2001; U.S. S No. 60/288,590, filed May 4, 2001; and U.S. S No. 60/294,443, filed May 29, 2001, all of which are incorporated herein by reference in their entirety.

### FIELD OF THE INVENTION

M-The invention relates to the identification of nucleic acid and protein expression profiles and nucleic acids, products, and antibodies thereto that are involved in breast cancer; and to the use of such expression profiles and compositions in the diagnosis, prognosis and therapy of breast cancer. The invention further relates to methods for identifying and using agents and/or targets that inhibit breast cancer.

### BACKGROUND OF THE INVENTION

Breast cancer is one of the most frequently diagnosed cancers and the second leading cause of female cancer death in North America and northern Europe, with lung cancer being the leading cause. Lifetime incidence of the disease in the United States is one-in-eight, with a 1-in-29 lifetime risk of dying from breast cancer. Early detection of breast cancer, using mammography, clinical breast examination, and self breast examination, has dramatically improved the treatment of the disease, although sensitivity is still major concern, as mammographic sensitivity has been estimated at only 60%-90%. Treatment of breast cancer consists largely of surgical lumpectomy or mastectomy, radiation therapy, anti-hormone therapy, and/or chemotherapy. Although many breast cancer patients are effectively treated, the current therapies can all induce serious side effects which diminish quality of life. Deciding on a particular course of treatment is typically based on a variety of prognostic parameters and markers (Fitzgibbons et al., 2000, Arch. Pathol. Lab. Med. 124:966-978; Hamilton and Piccart, 2000, Ann. Oncol. 11:647-663), including genetic predispostion markers BRCA-1 and BRCA-2 (Robson, 2000, J. Clin. Oncol. 18:113sup-118sup).

Imaging of breast cancer for diagnosis has been problematic and limited. In addition, dissemination of tumor cells (metastases) to locoregional lymph nodes is an important prognostic factor; five year survival rates drop from 80 percent in patients with no lymph node metastases to 45 to 50 percent in those patients who do have lymph node metastases. A recent report showed that micrometastases can be detected from lymph nodes using reverse transcriptase-PCR methods based on the presence of mRNA for carcinoembryonic antigen, which has previously been shown to be present in the vast majority of breast cancers but not in normal tissues. Liefers et al., New England J. of Med. 339(4):223 (1998).

The identification of novel therapeutic targets and diagnostic markers is essential for improving the current treatment of breast cancer patients. Recent advances in molecular medicine have increased the interest in tumor-specific cell surface antigens that could serve as targets for various immunotherapeutic or small molecule strategies. Antigens suitable for immunotherapeutic strategies should be highly expressed in cancer tissues and ideally not expressed in normal adult tissues. Expression in tissues that are dispensable for life, however, may be tolerated. Examples of such antigens include Her2/neu and the B-cell antigen CD20. Humanized monclonal antibodies directed to Her2/neu (Herceptin®/trastuzumab) are currently in use for the treatment of metastatic breast cancer (Ross and Fletcher, 1998, Stem Cells 16:413-428). Similarly, anti-CD20 monoclonal antibodies (Rituxin®/rituximab) are used to effectively treat non-Hodgekin's lymphoma (Maloney et al., 1997, Blood 90:2188-2195; Leget and Czuczman, 1998, Curr. Opin. Oncol. 10:548-551).

Other potential immunotherapeutic targets have been identified for breast cancer. One such target is polymorphic epithelial mucin (MUC1). MUC1 is a transmembrane protein, present at the apical surface of glandular epithelial cells. It is often overexpressed in breast cancer, and typically exhibits an altered glycosylation pattern, resulting in an antigenically distinct molecule, and is in early clinical trials as a vaccine target (Gilewski et al., 2000, Clin. Cancer Res. 6:1693-1701; Scholl et al., 2000, J. Immunother. 23:570-580). The tumor-expressed protein is often cleaved into the circulation, where it is detectable as the tumor marker, CA 15-3 (Bon et al., 1997, Clin. Chem. 43:585-593). However, many patients have tumors that express neither HER2 nor MUC-1; therefore, it is clear that other targets need to be identified to manage localized and metastatic disease. Many other genes have been reported to be overexpressed in breast cancer, such as EGFR (Sainsbury et al., 1987, Lancet 1(8547):1398-1402), c-erbB3 (Naidu et al., 1988, Br. J. Cancer 78:1385-1390), FGFR2 (Penault-Llorca et al., 1991, Int. J. Cancer 61:170-176), PKW (Preiherr et al., 2000, Anticancer Res. 20:2255-2264), MTA1 (Nawa et al., 2000, J. Cell Biochem. 79:202-212), breast cancer associated gene 1 (Kurt et al., 2000, Breast Cancer Res. Treat. 59:41-48). Although monoclonal antibodies to the protein products of some of these overexpressed genes have been reported (for review, see Green et al., 2000, Cancer Treat. Rev. 26:269-286), none are currently approved for breast cancer therapy in the US.

Disclosures of certain genes and ESTs described as being expressed in breast cancer are found in international patent applications WO-99/33869, WO-97/25426, WO-97/02280 and WO-00/55173, WO-98/45328 and WO-00/22130. Similarly, genes and ESTs described as being expressed in breast cancer are disclosed in U.S. Pat. Nos. 5,759,776 and 5,693,522. The utility of such genes is described in each of these publications, and their disclosures are incorporated herein in their entirety.

While industry and academia have identified novel sequences, there has not been an equal effort exerted to identify the function of these novel sequences. The elucidation of a role for novel proteins and compounds in disease states for identification of therapeutic targets and diagnostic markers is essential for improving the current treatment of breast cancer patients. Accordingly, provided herein are molecular targets for therapeutic intervention in breast and other cancers. Additionally, provided herein are methods that can be used in diagnosis and prognosis of breast cancer. Further provided are methods that can be used to screen candidate bioactive agents for the ability to modulate breast cancer.

### SUMMARY OF THE INVENTION

The present invention therefore provides nucleotide sequences of genes that are up- and down-regulated in breast cancer cells. Such genes are useful for diagnostic purposes, and also as targets for screening for therapeutic compounds that modulate breast cancer, such as hormones or antibodies. Other aspects of the invention will become apparent to the skilled artisan by the following description of the invention.

In one aspect, the present invention provides a method of detecting a breast cancer-associated transcript in a cell from a patient, the method comprising contacting a biological sample from the patient with a polynucleotide that selectively hybridizes to a sequence at least 80% identical to a sequence as shown in Tables 1-25.

In one embodiment, the present invention provides a method of determining the level of a breast cancer associated transcript in a cell from a patient.

In one embodiment, the present invention provides a method of detecting a breast cancer-associated transcript in a cell from a patient, the method comprising contacting a biological sample from the patient with a polynucleotide that selectively hybridizes to a sequence at least 80% identical to a sequence as shown in Tables 1-25.

In one embodiment, the polynucleotide selectively hybridizes to a sequence at least 95% identical to a sequence as shown in Tables 1-25.

In one embodiment, the biological sample is a tissue sample. In another embodiment, the biological sample comprises isolated nucleic acids, e.g., mRNA.

In one embodiment, the polynucleotide is labeled, e.g., with a fluorescent label.

In one embodiment, the polynucleotide is immobilized on a solid surface.

In one embodiment, the patient is undergoing a therapeutic regimen to treat breast cancer. In another embodiment, the patient is suspected of having metastatic breast cancer.

In one embodiment, the patient is a human.

In one embodiment, the breast cancer associated transcript is mRNA.

In one embodiment, the method further comprises the step of amplifying nucleic acids before the step of contacting the biological sample with the polynucleotide.

In another aspect, the present invention provides a method of monitoring the efficacy of a therapeutic treatment of breast cancer, the method comprising the steps of: (i) providing a biological sample from a patient undergoing the therapeutic treatment; and (ii) determining the level of a breast cancer-associated transcript in the biological sample by contacting the biological sample with a polynucleotide that selectively hybridizes to a sequence at least 80% identical to a sequence as shown in Tables 1-25, thereby monitoring the efficacy of the therapy. In a further embodiment, the patient has metastatic breast cancer. In a further embodiment, the patient has a drug resistant form of breast cancer.

In one embodiment, the method further comprises the step of: (iii) comparing the level of the breast cancer-associated transcript to a level of the breast cancer-associated transcript in a biological sample from the patient prior to, or earlier in, the therapeutic treatment.

Additionally, provided herein is a method of evaluating the effect of a candidate breast cancer drug comprising administering the drug to a patient and removing a cell sample from the patient. The expression profile of the cell is then determined. This method may further comprise comparing the expression profile to an expression profile of a healthy individual. In a preferred embodiment, said expression profile includes a gene of Tables 1-25.

In one aspect, the present invention provides an isolated nucleic acid molecule consisting of a polynucleotide sequence as shown in Tables 1-25.

In one embodiment, an expression vector or cell comprises the isolated nucleic acid.

In one aspect, the present invention provides an isolated polypeptide which is encoded by a nucleic acid molecule having polynucleotide sequence as shown in Tables 1-25.

In another aspect, the present invention provides an antibody that specifically binds to an isolated polypeptide which is encoded by a nucleic acid molecule having polynucleotide sequence as shown in Tables 1-25.

In one embodiment, the antibody is conjugated to an effector component, e.g., a fluorescent label, a radioisotope or a cytotoxic chemical.

In one embodiment, the antibody is an antibody fragment. In another embodiment, the antibody is humanized.

In one aspect, the present invention provides a method of detecting a breast cancer cell in a biological sample from a patient, the method comprising contacting the biological sample with an antibody as described herein.

In another aspect, the present invention provides a method of detecting antibodies specific to breast cancer in a patient, the method comprising contacting a biological sample from the patient with a polypeptide encoded by a nucleic acid comprising a sequence from Tables 1-25.

In another aspect, the present invention provides a method for identifying a compound that modulates a breast cancer-associated polypeptide, the method comprising the steps of: (i) contacting the compound with a breast cancer-associated polypeptide, the polypeptide encoded by a polynucleotide that selectively hybridizes to a sequence at least 80% identical to a sequence as shown in Tables 1-25; and (ii) determining the functional effect of the compound upon the polypeptide.

In one embodiment, the functional effect is a physical effect, an enzymatic effect, or a chemical effect.

In one embodiment, the polypeptide is expressed in a eukaryotic host cell or cell membrane. In another embodiment, the polypeptide is recombinant.

In one embodiment, the functional effect is determined by measuring ligand binding to the polypeptide.

In another aspect, the present invention provides a method of inhibiting proliferation of a breast cancer-associated cell to treat breast cancer in a patient, the method comprising the step of administering to the subject a therapeutically effective amount of a compound identified as described herein.

In one embodiment, the compound is an antibody.

In another aspect, the present invention provides a drug screening assay comprising the steps of: (i) administering a test compound to a mammal having breast cancer or to a cell sample isolated therefrom; (ii) comparing the level of gene expression of a polynucleotide that selectively hybridizes to a sequence at least 80% identical to a sequence as shown in Tables 1-25 in a treated cell or mammal with the level of gene expression of the polynucleotide in a control cell sample or mammal, wherein a test compound that modulates the level of expression of the polynucleotide is a candidate for the treatment of breast cancer.

In one embodiment, the control is a mammal with breast cancer or a cell sample therefrom that has not been treated with the test compound. In another embodiment, the control is a normal cell or mammal.

In one embodiment, the test compound is administered in varying amounts or concentrations. In another embodiment, the test compound is administered for varying time periods. In another embodiment, the comparison can occur after addition or removal of the drug candidate.

In one embodiment, the levels of a plurality of polynucleotides that selectively hybridize to a sequence at least 80% identical to a sequence as shown in Tables 1-25 are individually compared to their respective levels in a control cell sample or mammal. In a preferred embodiment the plurality of polynucleotides is from three to ten.

In another aspect, the present invention provides a method for treating a mammal having breast cancer comprising administering a compound identified by the assay described herein.

In another aspect, the present invention provides a pharmaceutical composition for treating a mammal having breast cancer, the composition comprising a compound identified by the assay described herein and a physiologically acceptable excipient.

In one aspect, the present invention provides a method of screening drug candidates by providing a cell expressing a gene that is up- and down-regulated as in a breast cancer. In one embodiment, a gene is selected from Tables 1-25. The method further includes adding a drug candidate to the cell and determining the effect of the drug candidate on the expression of the expression profile gene.

In one embodiment, the method of screening drug candidates includes comparing the level of expression in the absence of the drug candidate to the level-of expression in the presence of the drug candidate, wherein the concentration of the drug candidate can vary when present, and wherein the comparison can occur after addition or removal of the drug candidate. In a preferred embodiment, the cell expresses at least two expression profile genes. The profile genes may show an increase or decrease.

Also provided is a method of evaluating the effect of a candidate breast cancer drug comprising administering the drug to a transgenic animal expressing or over-expressing the breast cancer modulatory protein, or an animal lacking the breast cancer modulatory protein, for example as a result of a gene knockout.

Moreover, provided herein is a biochip comprising one or more nucleic acid segments of Tables 1-25, wherein the biochip comprises fewer than 1000 nucleic acid probes. Preferably, at least two nucleic acid segments are included. More preferably, at least three nucleic acid segments are included.

Furthermore, a method of diagnosing a disorder associated with breast cancer is provided. The method comprises determining the expression of a gene of Tables 1-25, preferably a gene of Table 25, in a first tissue type of a first individual, and comparing the distribution to the expression of the gene from a second normal tissue type from the first individual or a second unaffected individual. A difference in the expression indicates that the first individual has a disorder associated with breast cancer.

In a further embodiment, the biochip also includes a polynucleotide sequence of a gene that is not up- and down-regulated in breast cancer.

In one embodiment a method for screening for a bioactive agent capable of interfering with the binding of a breast cancer modulating protein (breast cancer modulatory protein) or a fragment thereof and an antibody which binds to said breast cancer modulatory protein or fragment thereof. In a preferred embodiment, the method comprises combining a breast cancer modulatory protein or fragment thereof, a candidate bioactive agent and an antibody which binds to said breast cancer modulatory protein or fragment thereof. The method further includes determining the binding of said breast cancer modulatory protein or fragment thereof and said antibody. Wherein there is a change in binding, an agent is identified as an interfering agent. The interfering agent can be an agonist or an antagonist. Preferably, the agent inhibits breast cancer.

Also provided herein are methods of eliciting an immune response in an individual. In one embodiment a method provided herein comprises administering to an individual a composition comprising a breast cancer modulating protein, or a fragment thereof. In another embodiment, the protein is encoded by a nucleic acid selected from those of Tables 1-25.

Further provided herein are compositions capable of eliciting an immune response in an individual. In one embodiment, a composition provided herein comprises a breast cancer modulating protein, preferably encoded by a nucleic acid of Tables 1-25, more preferably of Table 25, or a fragment thereof, and a pharmaceutically acceptable carrier. In another embodiment, said composition comprises a nucleic acid comprising a sequence encoding a breast cancer modulating protein, preferably selected from the nucleic acids of Tables 1-25, and a pharmaceutically acceptable carrier.

Also provided are methods of neutralizing the effect of a breast cancer protein, or a fragment thereof, comprising contacting an agent specific for said protein with said protein in an amount sufficient to effect neutralization. In another embodiment, the protein is encoded by a nucleic acid selected from those of Tables 1-25.

In another aspect of the invention, a method of treating an individual for breast cancer is provided. In one embodiment, the method comprises administering to said individual an inhibitor of a breast cancer modulating protein. In another embodiment, the method comprises administering to a patient having breast cancer an antibody to a breast cancer modulating protein conjugated to a therapeutic moiety. Such a therapeutic moiety can be a cytotoxic agent or a radioisotope.

### DETAILED DESCRIPTION OF THE INVENTION

In accordance with the objects outlined above, the present invention provides novel methods for diagnosis and prognosis evaluation for breast cancer (PC), including metastatic breast cancer, as well as methods for screening for compositions which modulate breast cancer. Also provided are methods for treating breast cancer.

Tables 1-24B provide unigene cluster identification numbers for the nucleotide sequence of genes that exhibit increased or decreased expression in breast cancer samples. Tables 4, 5, 6, 7, 8, 9, 10, 11, 12, 13, 18, 19, 20, 21, and 22 list those genes that are up-regulated in breast cancer cells. Table 14 lists those genes that are highly upregulated in breast cancer cells. Table 1, 2, 3, 15, and 23 list genes that are down-regulated in breast cancer cells and Table 16, lists genes that are highly down-regulated in breast cancer genes. The Tables also provide an exemplar accession number that provides a nucleotide sequence that is part of the unigene cluster.

Definitions

The term “breast cancer protein” or “breast cancer polynucleotide” or “breast cancer-associated transcript” refers to nucleic acid and polypeptide polymorphic variants, alleles, mutants, and interspecies homologues that: (1) have a nucleotide sequence that has greater than about 60% nucleotide sequence identity, 65%, 70%, 75%, 80%, 85%, 90%, preferably 91%, 92%, 93%, 94%, 95%, 96%, 97%, 98% or 99% or greater nucleotide sequence identity, preferably over a region of over a region of at least about 25, 50, 100, 200, 500, 1000, or more nucleotides, to a nucleotide sequence of or associated with a gene of Tables 1-25; (2) bind to antibodies, e.g., polyclonal antibodies, raised against an immunogen comprising an amino acid sequence encoded by a nucleotide sequence of or associated with a gene of Tables 1-25, and conservatively modified variants thereof; (3) specifically hybridize under stringent hybridization conditions to a nucleic acid sequence, or the complement thereof of Tables 1-25 and conservatively modified variants thereof or (4) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino sequence identity, preferably over a region of over a region of at least about 25, 50, 100, 200, 500, 1000, or more amino acid, to an amino acid sequence encoded by a nucleotide sequence of or associated with a gene of Tables 1-25. A polynucleotide or polypeptide sequence is typically from a mammal including, but not limited to, primate, e.g., human; rodent, e.g., rat, mouse, hamster; cow, pig, horse, sheep, or other mammal. A “breast cancer polypeptide” and a “breast cancer polynucledtide,” include both naturally occurring or recombinant forms.

A “full length” breast cancer protein or nucleic acid refers to a breast cancer polypeptide or polynucleotide sequence, or a variant thereof, that contains all of the elements normally contained in one or more naturally occurring, wild type breast cancer polynucleotide or polypeptide sequences. The “full length” may be prior to, or after, various stages of post-translation processing or splicing, including alternative splicing.

“Biological sample” as used herein is a sample of biological tissue or fluid that contains nucleic acids or polypeptides, e.g., of a breast cancer protein, polynucleotide or transcript. Such samples include, but are not limited to, tissue isolated from primates, e.g., humans, or rodents, e.g., mice, and rats. Biological samples may also include sections of tissues such as biopsy and autopsy samples, frozen sections taken for histologic purposes, blood, plasma, serum, sputum, stool, tears, mucus, hair, skin, etc. Biological samples also include explants and primary and/or transformed cell cultures derived from patient tissues. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

“Providing a biological sample” means to obtain a biological sample for use in methods described in this invention. Most often, this will be done by removing a sample of cells from an animal, but can also be accomplished by using previously isolated cells (e.g., isolated by another person, at another time, and/or for another purpose), or by performing the methods of the invention in vivo. Archival tissues, having treatment or outcome history, will be particularly useful.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions, as well as naturally occurring, e.g., polymorphic or allelic variants, and man-made variants. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of one of the number of contiguous positions selected from the group consisting typically of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Preferred examples of algorithms that are suitable for determining percent sequence identity and sequence similarity include the BLAST and BLAST 2.0 algorithms, which are described in Altschul et al., Nuc. Acids Res. 25:3389-3402 (1977) and Altschul et al., J. Mol. Biol. 215:403-410 (1990). BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=-4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N-4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001. Log values may be large negative numbers, e.g., 5, 10, 20, 30, 40, 40, 70, 90, 110, 150, 170, etc.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, e.g., where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequences.

A “host cell” is a naturally occurring cell or a transformed cell that contains an expression vector and supports the replication or expression of the expression vector. Host cells may be cultured cells, explants, cells in vivo, and the like. Host cells may be prokaryotic cells such as E. coli, or eukaryotic cells such as yeast, insect, amphibian, or mammalian cells such as CHO, HeLa, and the like (see, e.g., the American Type Culture Collection catalog or web site, www.atcc.org).

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. In particular, an isolated nucleic acid is separated from some open reading frames that naturally flank the gene and encode proteins other than protein encoded by the gene. The term “purified” in some embodiments denotes that a nucleic acid or protein gives rise to essentially one band in an electrophoretic gel. Preferably, it means that the nucleic acid or protein is at least 85% pure, more preferably at least 95% pure, and most preferably at least 99% pure. “Purify” or “purification” in other embodiments means removing at least one contaminant from the composition to be purified. In this sense, purification does not require that the purified compound be homogenous, e.g., 100% pure.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an α carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the-amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor & Schimmel, Biophysical Chemistry Part I. The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that often form a compact unit of the polypeptide and are typically 25 to approximately 500 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed, usually by the noncovalent association of independent tertiary units. Anisotropic terms are also known as energy terms.

“Nucleic acid” or “oligonucleotide” or “polynucleotide” or grammatical equivalents used herein means at least two nucleotides covalently linked together. Oligonucleotides are typically from about 5, 6, 7, 8, 9, 10, 12, 15, 25, 30, 40, 50 or more nucleotides in length, up to about 100 nucleotides in length. Nucleic acids and polynucleotides are a polymers of any length, including longer lengths, e.g., 200, 300, 500, 1000, 2000, 3000, 5000, 7000, 10,000, etc. A nucleic acid of the present invention will generally contain phosphodiester bonds, although in some cases, nucleic acid analogs are included that may have alternate backbones, comprising, e.g., phosphoramidate, phosphorothioate, phosphorodithioate, or O-methylphophoroamidite linkages (see Eckstein, Oligonucleotides and Analogues: A Practical Approach, Oxford University Press); and

peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g. to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

A variety of references disclose such nucleic acid analogs, including, for example, phosphoramidate (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within one definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference.

Particularly preferred are peptide nucleic acids (PNA) which includes peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tₘ) for mismatched versus perfectly matched basepairs. DNA and RNA typically exhibit a 2-4° C. drop in Tₘ for an internal mismatch. With the non-ionic PNA backbone, the drop is closer to 7-9° C. Similarly, due to their non-ionic nature, hybridization of the bases attached to these backbones is relatively insensitive to salt concentration. In addition, PNAs are not degraded by cellular enzymes, and thus can be more stable.

The nucleic acids may be single stranded or double stranded, as specified, or contain portions of both double stranded or single stranded sequence. As will be appreciated by those in the art, the depiction of a single strand also defines the sequence of the complementary strand; thus the sequences described herein also provide the complement of the sequence. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. “Transcript” typically refers to a naturally occurring RNA, e.g., a pre-mRNA, hnRNA, or mRNA. As used herein, the term “nucleoside” includes nucleotides and nucleoside and nucleotide analogs, and modified nucleosides such as amino modified nucleosides. In addition, “nucleoside” includes non-naturally occurring analog structures. Thus, e.g. the individual units of a peptide nucleic acid, each containing a base, are referred to herein as a nucleoside.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into the peptide or used to detect antibodies specifically reactive with the peptide. The labels may be incorporated into the breast cancer nucleic acids, proteins and antibodies at any position. Any method known in the art for conjugating the antibody to the label may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

An “effector” or “effector moiety” or “effector component” is a molecule that is bound (or linked, or conjugated), either covalently, through a linker or a chemical bond, or noncovalently, through ionic, van der Waals, electrostatic, or hydrogen bonds, to an antibody. The “effector” can be a variety of molecules including, e.g., detection moieties including radioactive compounds, fluorescent compounds, an enzyme or substrate, tags such as epitope tags, a toxin; activatable moieties, a chemotherapeutic agent; a lipase; an antibiotic; or a radioisotope emitting “hard” e.g., beta radiation.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe. Alternatively, method using high affinity interactions may achieve the same results where one of a pair of binding partners binds to the other, e.g., biotin, streptavidin.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not functionally interfere with hybridization. Thus, e.g., probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence. Diagnosis or prognosis may be based at the genomic level, or at the level of RNA or protein expression.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operably linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

The term “heterologous” when used with reference to portions of a nucleic acid indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous protein will often refer to two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such assa promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

Nucleic acids that do not hybridize to each other under stringent conditions are still substantially identical if the polypeptides which they encode are substantially identical. This occurs, e.g., when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

The phrase “functional effects” in the context of assays for testing compounds that modulate activity of a breast cancer protein includes the determination of a parameter that is indirectly or directly under the influence of the breast cancer protein or nucleic acid, e.g., a functional, physical, or chemical effect, such as the ability to decrease breast cancer. It includes ligand binding activity; cell growth on soft agar; anchorage dependence; contact inhibition and density limitation of growth; cellular proliferation; cellular transformation; growth factor or serum dependence; tumor specific marker levels; invasiveness into Matrigel; tumor growth and metastasis in vivo; mRNA and protein expression in cells undergoing metastasis, and other characteristics of breast cancer cells. “Functional effects” include in vitro, in vivo, and ex vivo activities.

By “determining the functional effect” is meant assaying for a compound that increases or decreases a parameter that is indirectly or directly under the influence of a breast cancer protein sequence, e.g., functional, enzymatic, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties for the protein, measuring inducible markers or transcriptional activation of the breast cancer protein; measuring binding activity or binding assays, e.g. binding to antibodies or other ligands, and measuring cellular proliferation. Determination of the functional effect of a compound on breast cancer can also be performed using breast cancer assays known to those of skill in the art such as an in vitro assays, e.g., cell growth on soft agar; anchorage dependence; contact inhibition and density limitation of growth; cellular proliferation; cellular transformation; growth factor or serum dependence; tumor specific marker levels; invasiveness into Matrigel; tumor growth and metastasis in vivo; mRNA and protein expression in cells undergoing metastasis, and other characteristics of breast cancer cells. The functional effects can be evaluated by many means known to those skilled in the art, e.g., microscopy for quantitative or qualitative measures of alterations in morphological features, measurement of changes in RNA or protein levels for breast cancer-associated sequences, measurement of RNA stability, identification of downstream or reporter gene expression (CAT, luciferase, β-gal, GFP and the like), e.g., via chemiluminescence, fluorescence, colorimetric reactions, antibody binding, inducible markers, and ligand binding assays.

“Inhibitors”, “activators”, and “modulators” of breast cancer polynucleotide and polypeptide sequences are used to refer to activating, inhibitory, or modulating molecules or compounds identified using in vitro and in vivo assays of breast cancer polynucleotide and polypeptide sequences. Inhibitors are compounds that, e.g., bind to, partially or totally block activity, decrease, prevent, delay activation, inactivate, desensitize, or down regulate the activity or expression of breast cancer proteins, e.g., antagonists. Antisense nucleic acids may seem to inhibit expression and subsequent function of the protein. “Activators” are compounds that increase, open, activate, facilitate, enhance activation, sensitize, agonize, or up regulate breast cancer protein activity. Inhibitors, activators, or modulators also include genetically modified versions of breast cancer proteins, e.g., versions with altered activity, as well as naturally occurring and synthetic ligands, antagonists, agonists, antibodies, small chemical molecules and the like. Such assays for inhibitors and activators include, e.g., expressing the breast cancer protein in vitro, in cells, or cell membranes, applying putative modulator compounds, and then determining the functional effects on activity, as described above. Activators and inhibitors of breast cancer can also be identified by incubating breast cancer cells with the test compound and determining increases or decreases in the expression of 1 or more breast cancer proteins, e.g., 1, 2, 3, 4, 5, 10, 15, 20, 25, 30, 40, 50 or more breast cancer proteins, such as breast cancer proteins encoded by the sequences set out in Tables 1-25.

Samples or assays comprising breast cancer proteins that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition of a polypeptide is achieved when the activity value relative to the control is about 80%, preferably 50%, more preferably 25-0%. Activation of a breast cancer polypeptide is achieved when the activity value relative to the control (untreated with activators) is 110%, more preferably 150%, more preferably 200-500% (i.e., two to five fold higher relative to the control), more preferably 1000-3000% higher.

The phrase “changes in cell growth” refers to any change in cell growth and proliferation characteristics in vitro or in vivo, such as formation of foci, anchorage independence, semi-solid or soft agar growth, changes in contact inhibition and density limitation of growth, loss of growth factor or serum requirements, changes in cell morphology, gaining or losing immortalization, gaining or losing tumor specific markers, ability to form or suppress tumors when injected into suitable animal hosts, and/or immortalization of the cell. See, e.g., Freshney, Culture of Animal Cells a Manual of Basic Technique pp. 231-241 (3^{rd} ed. 1994).

“Tumor cell” refers to precancerous, cancerous, and normal cells in a tumor.

“Cancer cells,” “transformed” cells or “transformation” in tissue culture, refers to spontaneous or induced phenotypic changes that do not necessarily involve the uptake of new genetic material. Although transformation can arise from infection with a transforming virus and incorporation of new genomic DNA, or uptake of exogenous DNA, it can also arise spontaneously or following exposure to a carcinogen, thereby mutating an endogenous gene. Transformation is associated with phenotypic changes, such as immortalization of cells, aberrant growth control, nonmorphological changes, and/or malignancy (see, Freshney, Culture of Animal Cells a Manual of Basic Technique (3^{rd} ed. 1994)).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody or its functional equivalent will be most critical in specificity and affinity of binding. See Paul, Fundamental Immunology.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, e.g., pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985); Coligan, Current Protocols in Immunology (1991); Harlow & Lane, Antibodies, A Laboratory Manual (1988); and Goding, Monoclonal Antibodies: Principles and Practice (2d ed. 1986)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

Identification of Breast Cancer-Associated Sequences

In one aspect, the expression levels of genes are determined in different patient samples for which diagnosis information is desired, to provide expression profiles. An expression profile of a particular sample is essentially a “fingerprint” of the state of the sample; while two states may have any particular gene similarly expressed, the evaluation of a number of genes simultaneously allows the generation of a gene expression profile that is characteristic of the state of the cell. That is, normal tissue (e.g., normal breast or other tissue) may be distinguished from cancerous or metastatic cancerous tissue of the breast, or breast cancer tissue or metastatic breast cancerous tissue can be compared with tissue samples of breast and other tissues from surviving cancer patients. By comparing expression profiles of tissue in known different breast cancer states, information regarding which genes are important (including both up- and down-regulation of genes) in each of these states is obtained.

The identification of sequences that are differentially expressed in breast cancer versus non-breast cancer tissue allows the use of this information in a number of ways. For example, a particular treatment regime may be evaluated: does a chemotherapeutic drug act to down-regulate breast cancer, and thus tumor growth or recurrence, in a particular patient. Similarly, diagnosis and treatment outcomes may be done or confirmed by comparing patient samples with the known expression profiles. Metastatic tissue can also be analyzed to determine the stage of breast cancer in the tissue. Furthermore, these gene expression profiles (or individual genes) allow screening of drug candidates with an eye to mimicking or altering a particular expression profile; e.g., screening can be done for drugs that suppress the breast cancer expression profile. This may be done by making biochips comprising sets of the important breast cancer genes, which can then be used in these screens. These methods can also be done on the protein basis; that is, protein expression levels of the breast cancer proteins can be evaluated for diagnostic purposes or to screen candidate agents. In addition, the breast cancer nucleic acid sequences can be administered for gene therapy purposes, including the administration of antisense nucleic acids, or the breast cancer proteins (including antibodies and other modulators thereof) administered as therapeutic drugs.

Thus the present invention provides nucleic acid and protein sequences that are differentially expressed in breast cancer, herein termed “breast cancer sequences.” As outlined below, breast cancer sequences include those that are up-regulated (i.e., expressed at a higher level) in breast cancer, as well as those that are down-regulated (i.e., expressed at a lower level). In a preferred embodiment, the breast cancer sequences are from humans; however, as will be appreciated by those in the art, breast cancer sequences from other organisms may be useful in animal models of disease and drug evaluation; thus, other breast cancer sequences are provided, from vertebrates, including mammals, including rodents (rats, mice, hamsters, guinea pigs, etc.), primates, farm animals (including sheep, goats, pigs, cows, horses, etc.) and pets, e.g., (dogs, cats, etc.). Breast cancer sequences from other organisms may be obtained using the techniques outlined below.

Breast cancer sequences can include both nucleic acid and amino acid sequences. As will be appreciated by those in the art and is more fully outlined below, breast cancer nucleic acid sequences are useful in a variety of applications, including diagnostic applications, which will detect naturally occurring nucleic acids, as well as screening applications; e.g., biochips comprising nucleic acid probes or PCR microtiter plates with selected probes to the breast cancer sequences can be generated.

A breast cancer sequence can be initially identified by substantial nucleic acid and/or amino acid sequence homology to the breast cancer sequences outlined herein. Such homology can be based upon the overall nucleic acid or amino acid sequence, and is generally determined as outlined below, using either homology programs or hybridization conditions.

For identifying breast cancer-associated sequences, the breast cancer screen typically includes comparing genes identified in different tissues, e.g., normal and cancerous tissues, or tumor tissue samples from patients who have metastatic disease vs. non metastatic tissue. Other suitable tissue comparisons include comparing breast cancer samples with metastatic cancer samples from other cancers, such as lung, breast, gastrointestinal cancers, ovarian, etc. Samples of different stages of breast cancer, e.g., survivor tissue, drug resistant states, and tissue undergoing metastasis, are applied to biochips comprising nucleic acid probes. The samples are first microdissected, if applicable, and treated as is known in the art for the preparation of mRNA. Suitable biochips are commercially available, e.g. from Affymetrix. Gene expression profiles as described herein are generated and the data analyzed.

In one embodiment, the genes showing changes in expression as between normal and disease states are compared to genes expressed in other normal tissues, preferably normal breast, but also including, and not limited to lung, heart, brain, liver, breast, kidney, muscle, colon, small intestine, large intestine, spleen, bone and placenta. In a preferred embodiment, those genes identified during the breast cancer screen that are expressed in any significant amount in other tissues are removed from the profile, although in some embodiments, this is not necessary. That is, when screening for drugs, it is usually preferable that the target be disease specific, to minimize possible side effects.

In a preferred embodiment, breast cancer sequences are those that are up-regulated in breast cancer; that is, the expression of these genes is higher in the breast cancer tissue as compared to non-cancerous tissue. “Up-regulation” as used herein often means at least about a two-fold change, preferably at least about a three fold change, with at least about five-fold or higher being preferred. All unigene cluster identification numbers and accession numbers herein are for the GenBank sequence database and the sequences of the accession numbers are hereby expressly incorporated by reference. GenBank is known in the art, see, e.g., Benson, D A, et al., Nucleic Acids Research 26:1-7 (1998) and http://www.ncbi.nlm.nih.gov/. Sequences are also available in other databases, e.g., European Molecular Biology Laboratory (EMBL) and DNA Database of Japan (DDBJ). U.S. patent application Ser. No. 09/687,576, with the same assignee as the present application, further discloses related sequences, compositions, and methods of diagnosis and treatment of breast cancer is hereby expressly incorporated by reference.

In another preferred embodiment, breast cancer sequences are those that are down-regulated in the breast cancer; that is, the expression of these genes is lower in breast cancer tissue as compared to non-cancerous tissue (see, e.g., Tables 1, 2, 3, 15, 16 etc . . . ). “Down-regulation” as used herein often means at least about a two-fold change, preferably at least about a three fold change, with at least about five-fold or higher being preferred.

Informatics

The ability to identify genes that are over or under expressed in breast cancer can additionally provide high-resolution, high-sensitivity datasets which can be used in the areas of diagnostics, therapeutics, drug development, pharmacogenetics, protein structure, biosensor development, and other related areas. For example, the expression profiles can be used in diagnostic or prognostic evaluation of patients with breast cancer. Or as another example, subcellular toxicological information can be generated to better direct drug structure and activity correlation (see Anderson, Pharmaceutical Proteomics: Targets, Mechanism, and Function, paper presented at the IBC Proteomics conference, Coronado, Calif. (Jun. 11-12, 1998)). Subcellular toxicological information can also be utilized in a biological sensor device to predict the likely toxicological effect of chemical exposures and likely tolerable exposure thresholds (see U.S. Pat. No. 5,811,231). Similar advantages accrue from datasets relevant to other biomolecules and bioactive agents (e.g., nucleic acids, saccharides, lipids, drugs, and the like).

Thus, in another embodiment, the present invention provides a database that includes at least one set of assay data. The data contained in the database is acquired, e.g., using array analysis either singly or in a library format. The database can be in substantially any form in which data can be maintained and transmitted, but is preferably an electronic database. The electronic database of the invention can be maintained on any electronic device allowing for the storage of and access to the database, such as a personal computer, but is preferably distributed on a wide area network, such as the World Wide Web.

The focus of the present section on databases that include peptide sequence data is for clarity of illustration only. It will be apparent to those of skill in the art that similar databases can be assembled for any assay data acquired using an assay of the invention.

The compositions and methods for identifying and/or quantitating the relative and/or absolute abundance of a variety of molecular and macromolecular species from a biological sample undergoing breast cancer, i.e., the identification of breast cancer-associated sequences described herein, provide an abundance of information, which can be correlated with pathological conditions, predisposition to disease, drug testing, therapeutic monitoring, gene-disease causal linkages, identification of correlates of immunity and physiological status, among others. Although the data generated from the assays of the invention is suited for manual review and analysis, in a preferred embodiment, prior data processing using high-speed computers is utilized.

An array of methods for indexing and retrieving biomolecular information is known in the art. For example, U.S. Pat. Nos. 6,023,659 and 5,966,712 disclose a relational database system for storing biomolecular sequence information in a manner that allows sequences to be catalogued and searched according to one or more protein function hierarchies. U.S. Pat. No. 5,953,727 discloses a relational database having sequence records containing information in a format that allows a collection of partial-length DNA sequences to be catalogued and searched according to association with one or more sequencing projects for obtaining full-length sequences from the collection of partial length sequences. U.S. Pat. No. 5,706,498 discloses a gene database retrieval system for making a retrieval of a gene sequence similar to a sequence data item in a gene database based on the degree of similarity between a key sequence and a target sequence. U.S. Pat. No. 5,538,897 discloses a method using mass spectroscopy fragmentation patterns of peptides to identify amino acid sequences in computer databases by comparison of predicted mass spectra with experimentally-derived mass spectra using a closeness-of-fit measure. U.S. Pat. No. 5,926,818 discloses a multi-dimensional database comprising a functionality for multi-dimensional data analysis described as on-line analytical processing (OLAP), which entails the consolidation of projected and actual data according to more than one consolidation path or dimension. U.S. Pat. No. 5,295,261 reports a hybrid database structure in which the fields of each database record are divided into two classes, navigational and informational data, with navigational fields stored in a hierarchical topological map which can be viewed as a tree structure or as the merger of two or more such tree structures.

See also Mount et al., Bioinformatics (2001); Biological Sequence Analysis: Probabilistic Models of Proteins and Nucleic Acids (Durbin et al., eds., 1999); Bioinformatics: A Practical Guide to the Analysis of Genes and Proteins (Baxevanis & Oeullette eds., 1998)); Rashidi & Buehler, Bioinformatics: Basic Applications in Biological Science and Medicine (1999); Introduction to Computational Molecular Biology (Setubal et al., eds 1997); Bioinformatics: Methods and Protocols (Misener & Krawetz, eds, 2000); Bioinformatics: Sequence, Structure, and Databanks: A Practical Approach (Higgins & Taylor, eds., 2000); Brown, Bioinformatics: A Biologist's Guide to Biocomputing and the Internet (2001); Han & Kamber, Data Mining: Concepts and Techniques (2000); and Waterman, Introduction to Computational Biology: Maps, Sequences, and Genomes (1995).

The present invention provides a computer database comprising a computer and software for storing in computer-retrievable form assay data records cross-tabulated, e.g., with data specifying the source of the target-containing sample from which each sequence specificity record was obtained.

In an exemplary embodiment, at least one of the sources of target-containing sample is from a control tissue sample known to be free of pathological disorders. In a variation, at least one of the sources is a known pathological tissue specimen, e.g., a neoplastic lesion or another tissue specimen to be analyzed for breast cancer. In another variation, the assay records cross-tabulate one or more of the following parameters for each target species in a sample: (1) a unique identification code, which can include, e.g., a target molecular structure and/or characteristic separation coordinate (e.g., electrophoretic coordinates); (2) sample source; and (3) absolute and/or relative quantity of the target species present in the sample.

The invention also provides for the storage and retrieval of a collection of target data in a computer data storage apparatus, which can include magnetic disks, optical disks, magneto-optical disks, DRAM, SRAM, SGRAM, SDRAM, RDRAM, DDR RAM, magnetic bubble memory devices, and other data storage devices, including CPU registers and on-CPU data storage arrays. Typically, the target data records are stored as a bit pattern in an array of magnetic domains on a magnetizable medium or as an array of charge states or transistor gate states, such as an array of cells in a DRAM device (e.g., each cell comprised of a transistor and a charge storage area, which may be on the transistor). In one embodiment, the invention provides such storage devices, and computer systems built therewith, comprising a bit pattern encoding a protein expression fingerprint record comprising unique identifiers for at least 10 target data records cross-tabulated with target source.

When the target is a peptide or nucleic acid, the invention preferably provides a method for identifying related peptide or nucleic acid sequences, comprising performing a computerized comparison between a peptide or nucleic acid sequence assay record stored in or retrieved from a computer storage device or database and at least one other sequence. The comparison can include a sequence analysis or comparison algorithm or computer program embodiment thereof (e.g., FASTA, TFASTA, GAP, BESTFIT) and/or the comparison may be of the relative amount of a peptide or nucleic acid sequence in a pool of sequences determined from a polypeptide or nucleic acid sample of a specimen.

The invention also preferably provides a magnetic disk, such as an IBM-compatible (DOS, Windows, Windows95/98/2000, Windows NT, OS/2) or other format (e.g., Linux, SunOS, Solaris, AIX, SCO Unix, VMS, MV, Macintosh, etc.) floppy diskette or hard (fixed, Winchester) disk drive, comprising a bit pattern encoding data from an assay of the invention in a file format suitable for retrieval and processing in a computerized sequence analysis, comparison, or relative quantitation method.

The invention also provides a network, comprising a plurality of computing devices linked via a data link, such as an Ethernet cable (coax or 10BaseT), telephone line, ISDN line, wireless network, optical fiber, or other suitable signal transmission medium, whereby at least one network device (e.g., computer, disk array, etc.) comprises a pattern of magnetic domains (e.g., magnetic disk) and/or charge domains (e.g., an array of DRAM cells) composing a bit pattern encoding data acquired from an assay of the invention.

The invention also provides a method for transmitting assay data that includes generating an electronic signal on an electronic communications device, such as a modem, ISDN terminal adapter, DSL, cable modem, ATM switch, or the like, wherein the signal includes (in native or encrypted format) a bit pattern encoding data from an assay or a database comprising a plurality of assay results obtained by the method of the invention.

In a preferred embodiment, the invention provides a computer system for comparing a query target to a database containing an array of data structures, such as an assay result obtained by the method of the invention, and ranking database targets based on the degree of identity and gap weight to the target data. A central processor is preferably initialized to load and execute the computer program for alignment and/or comparison of the assay results. Data for a query target is entered into the central processor via an I/O device. Execution of the computer program results in the central processor retrieving the assay data from the data file, which comprises a binary description of an assay result.

The target data or record and the computer program can be transferred to secondary memory, which is typically random access memory (e.g., DRAM, SRAM, SGRAM, or SDRAM). Targets are ranked according to the degree of correspondence between a selected assay characteristic (e.g., binding to a selected affinity moiety) and the same characteristic of the query target and results are output via an I/O device. For example, a central processor can be a conventional computer (e.g., Intel Pentium, PowerPC, Alpha, PA-8000, SPARC, MIPS 4400, MIPS 10000, VAX, etc.); a program can be a commercial or public domain molecular biology software package (e.g., UWGCG Sequence Analysis Software, Darwin); a data file can be an optical or magnetic disk, a data server, a memory device (e.g., DRAM, SRAM, SGRAM, SDRAM, EPROM, bubble memory, flash memory, etc.); an I/O device can be a terminal comprising a video display and a keyboard, a modem, an ISDN terminal adapter, an Ethernet port, a punched card reader, a magnetic strip reader, or other suitable I/O device.

The invention also preferably provides the use of a computer system, such as that described above, which comprises: (1) a computer; (2) a stored bit pattern encoding a collection of peptide sequence specificity records obtained by the methods of the invention, which may be stored in the computer; (3) a comparison target, such as a query target; and (4) a program for alignment and comparison, typically with rank-ordering of comparison results on the basis of computed similarity values.

Characteristics of Breast Cancer-Associated Proteins

Breast cancer proteins of the present invention may be classified as secreted proteins, transmembrane proteins or intracellular proteins. In one embodiment, the breast cancer protein is an intracellular protein. Intracellular proteins may be found in the cytoplasm and/or in the nucleus. Intracellular proteins are involved in all aspects of cellular function and replication (including, e.g., signaling pathways); aberrant expression of such proteins often results in unregulated or disregulated cellular processes (see, e.g., Molecular Biology of the Cell (Alberts, ed., 3rd ed., 1994). For example, many intracellular proteins have enzymatic activity such as protein kinase activity, protein phosphatase activity, protease activity, nucleotide cyclase activity, polymerase activity and the like. Intracellular proteins also serve as docking proteins that are involved in organizing complexes of proteins, or targeting proteins to various subcellular localizations, and are involved in maintaining the structural integrity of organelles.

An increasingly appreciated concept in characterizing proteins is the presence in the proteins of one or more motifs for which defined functions have been attributed. In addition to the highly conserved sequences found in the enzymatic domain of proteins, highly conserved sequences have been identified in proteins that are involved in protein-protein interaction. For example, Src-homology-2 (SH2) domains bind tyrosine-phosphorylated targets in a sequence dependent manner. PTB domains, which are distinct from SH2 domains, also bind tyrosine phosphorylated targets. SH3 domains bind to proline-rich targets. In addition, PH domains, tetratricopeptide repeats and WD domains to name only a few, have been shown to mediate protein-protein interactions. Some of these may also be involved in binding to phospholipids or other second messengers. As will be appreciated by one of ordinary skill in the art, these motifs can be identified on the basis of primary sequence; thus, an analysis of the sequence of proteins may provide insight into both the enzymatic potential of the molecule and/or molecules with which the protein may associate. One useful database is Pfam (protein families), which is a large collection of multiple sequence alignments and hidden Markov models covering many common protein domains. Versions are available via the internet from Washington University in St. Louis, the Sanger Center in England, and the Karolinska Institute in Sweden (see, e.g., Bateman et al., Nuc. Acids Res. 28:263-266 (2000); Sonnhammer et al., Proteins 28:405-420 (1997); Bateman et al., Nuc. Acids Res. 27:260-262 (1999); and Sonnhammer et al., Nuc. Acids Res. 26:320-322-(1998)).

In another embodiment, the breast cancer sequences are transmembrane proteins. Transmembrane proteins are molecules that span a phospholipid bilayer of a cell. They may have an intracellular domain, an extracellular domain, or both. The intracellular domains of such proteins may have a number of functions including those already described for intracellular proteins. For example, the intracellular domain may have enzymatic activity and/or may serve as a binding site for additional proteins. Frequently the intracellular domain of transmembrane proteins serves both roles. For example certain receptor tyrosine kinases have both protein kinase activity and SH2 domains. In addition, autophosphorylation of tyrosines on the receptor molecule itself, creates binding sites for additional SH2 domain containing proteins.

Transmembrane proteins may contain from one to many transmembrane domains. For example, receptor tyrosine kinases, certain cytokine receptors, receptor guanylyl cyclases and receptor serine/threonine protein kinases contain a single transmembrane domain. However, various other proteins including channels and adenylyl cyclases contain numerous transmembrane domains. Many important cell surface receptors such as G protein coupled receptors (GPCRs) are classified as “seven transmembrane domain” proteins, as they contain 7 membrane spanning regions. Characteristics of transmembrane domains include approximately 20 consecutive hydrophobic amino acids that F-5 may be followed by charged amino acids. Therefore, upon analysis of the amino acid sequence of a particular protein, the localization and number of transmembrane domains within the protein may be predicted (see, e.g. PSORT web site http://psort.nibb.acjp/). Important transmembrane protein receptors include, but are not limited to the insulin receptor, insulin-like growth factor receptor, human growth hormone receptor, glucose transporters, transferrin receptor, epidermal growth factor receptor, low density lipoprotein receptor, epidermal growth factor receptor, leptin receptor, interleukin receptors, e.g. IL-1 receptor, IL-2 receptor,

The extracellular domains of transmembrane proteins are diverse; however, conserved motifs are found repeatedly among various extracellular domains. Conserved structure and/or functions have been ascribed to different extracellular motifs. Many extracellular domains are involved in binding to other molecules. In one aspect, extracellular domains are found on receptors. Factors that bind the receptor domain include circulating ligands, which may be peptides, proteins, or small molecules such as adenosine and the like. For example, growth factors such as EGF, FGF and PDGF are circulating growth factors that bind to their cognate receptors to initiate a variety of cellular responses. Other factors include cytokines, mitogenic factors, neurotrophic factors and the like. Extracellular domains also bind to cell-associated molecules. In this respect, they mediate cell-cell interactions. Cell-associated ligands can be tethered to the cell, e.g., via a glycosylphosphatidylinositol (GPI) anchor, or may themselves be transmembrane proteins. Extracellular domains also associate with the extracellular matrix and contribute to the maintenance of the cell structure.

Breast cancer proteins that are transmembrane are particularly preferred in the present invention as they are readily accessible targets for immunotherapeutics, as are described herein. In addition, as outlined below, transmembrane proteins can be also useful in imaging modalities. Antibodies may be used to label such readily accessible proteins in situ. Alternatively, antibodies can also label intracellular proteins, in which case samples are typically permeablized to provide access to intracellular proteins.

It will also be appreciated by those in the art that a transmembrane protein can be made soluble by removing transmembrane sequences, e.g., through recombinant methods. Furthermore, transmembrane proteins that have been made soluble can be made to be secreted through recombinant means by adding an appropriate signal sequence.

In another embodiment, the breast cancer proteins are secreted proteins; the secretion of which can be either constitutive or regulated. These proteins have a signal peptide or signal sequence that targets the molecule to the secretory pathway. Secreted proteins are involved in numerous physiological events; by virtue of their circulating nature, they serve to transmit signals to various other cell types. The secreted protein may function in an autocrine manner (acting on the cell that secreted the factor), a paracrine manner (acting on cells in close proximity to the cell that secreted the factor) or an endocrine manner (acting on cells at a distance). Thus secreted molecules find use in modulating or altering numerous aspects of physiology. Breast cancer proteins that are secreted proteins are particularly preferred in the present invention as they serve as good targets for diagnostic markers, e.g., for blood, plasma, serum, or stool tests.

Use of Breast Cancer Nucleic Acids

As described above, breast cancer sequence is initially identified by substantial nucleic acid and/or amino acid sequence homology or linkage to the breast cancer sequences outlined herein. Such homology can be based upon the overall nucleic acid or amino acid sequence, and is generally determined as outlined below, using either homology programs or hybridization conditions. Typically, linked sequences on a mRNA are found on the same molecule.

The breast cancer nucleic acid sequences of the invention, e.g., the sequences in Tables 1-25, can be fragments of larger genes, i.e., they are nucleic acid segments. “Genes” in this context includes coding regions, non-coding regions, and mixtures of coding and non-coding regions. Accordingly, as will be appreciated by those in the art, using the sequences provided herein, extended sequences, in either direction, of the breast cancer genes can be obtained, using techniques well known in the art for cloning either longer sequences or the fall length sequences; see Ausubel, et al., supra. Much can be done by informatics and many sequences can be clustered to include multiple sequences corresponding to a single gene, e.g., systems such as UniGene (see, http://www.ncbi.nlm.nih.gov/UniGene/).

Once the breast cancer nucleic acid is identified, it can be cloned and, if necessary, its constituent parts recombined to form the entire breast cancer nucleic acid coding regions or the entire mRNA sequence. Once isolated from its natural source, e.g., contained within a plasmid or other vector or excised therefrom as a linear nucleic acid segment, the recombinant breast cancer nucleic acid can be further-used as a probe to identify and isolate other breast cancer nucleic acids, e.g., extended coding regions. It can also be used as a “precursor” nucleic acid to make modified or variant breast cancer nucleic acids and proteins.

The breast cancer nucleic acids of the present invention are used in several ways. In a first embodiment, nucleic acid probes to the breast cancer nucleic acids are made and attached to biochips to be used in screening and diagnostic methods, as outlined below, or for administration, e.g., for gene therapy, vaccine, and/or antisense applications. Alternatively, the breast cancer nucleic acids that include coding regions of breast cancer proteins can be put into expression vectors for the expression of breast cancer proteins, again for screening purposes or for administration to a patient.

In a preferred embodiment, nucleic acid probes to breast cancer nucleic acids (both the nucleic acid sequences outlined in the figures and/or the complements thereof) are made. The nucleic acid probes attached to the biochip are designed to be substantially complementary to the breast cancer nucleic acids, i.e. the target sequence (either the target sequence of the sample or to other probe sequences, e.g., in sandwich assays), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions, particularly high stringency conditions, as outlined herein.

A nucleic acid probe is generally single stranded but can be partially single and partially double stranded. The strandedness of the probe is dictated by the structure, composition, and properties of the target sequence. In general, the nucleic acid probes range from about 8 to about 100 bases long, with from about 10 to about 80 bases being preferred, and from about 30 to about 50 bases being particularly preferred. That is, generally whole genes are not used. In some embodiments, much longer nucleic acids can be used, up to hundreds of bases.

In a preferred embodiment, more than one probe per sequence is used, with either overlapping probes or probes to different sections of the target being used. That is, two, three, four or more probes, with three being preferred, are used to build in a redundancy for a particular target. The probes can be overlapping (i.e., have some sequence in common), or separate. In some cases, PCR primers may be used to amplify signal for higher sensitivity.

As will be appreciated by those in the art, nucleic acids can be attached or immobilized to a solid support in a wide variety of ways. By “immobilized” and grammatical equivalents herein is meant the association or binding between the nucleic acid probe and the solid support is sufficient to be stable under the conditions of binding, washing, analysis, and removal as outlined below. The binding can typically be covalent or non-covalent. By “non-covalent binding” and grammatical equivalents herein is meant one or more of electrostatic, hydrophilic, and hydrophobic interactions. Included in non-covalent binding is the covalent attachment of a molecule, such as, streptavidin to the support and the non-covalent binding of the biotinylated probe to the streptavidin. By “covalent binding” and grammatical equivalents herein is meant that the two moieties, the solid support and the probe, are attached by at least one bond, including sigma bonds, pi bonds and coordination bonds. Covalent bonds can be formed directly between the probe and the solid support or can be formed by a cross linker or by inclusion of a specific reactive group on either the solid support or the probe or both molecules. Immobilization may also involve a combination of covalent and non-covalent interactions.

In general, the probes are attached to the biochip in a wide variety of ways, as will be appreciated by those in the art. As described herein, the nucleic acids can either be synthesized first, with subsequent attachment to the biochip, or can be directly synthesized on the biochip.

The biochip comprises a suitable solid substrate. By “substrate” or “solid support” or other grammatical equivalents herein is meant a material that can be modified to contain discrete individual sites appropriate for the attachment or association of the nucleic acid probes and is amenable to at least one detection method. As will be appreciated by those in the art, the number of possible substrates are very large, and include, but are not limited to, glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses, plastics, etc. In general, the substrates allow optical detection and do not appreciably fluoresce. A preferred substrate is described in copending application entitled Reusable Low Fluorescent Plastic Biochip, U.S. application Ser. No. 09/270,214, filed Mar. 15, 1999, herein incorporated by reference in its entirety.

Generally the substrate is planar, although as will be appreciated by those in the art, other configurations of substrates may be used as well. For example, the probes may be placed on the inside surface of a tube, for flow-through sample analysis to minimize sample volume. Similarly, the substrate may be flexible, such as a flexible foam, including closed cell foams made of particular plastics.

In a preferred embodiment, the surface of the biochip and the probe may be derivatized with chemical functional groups for subsequent attachment of the two. Thus, e.g., the biochip is derivatized with a chemical functional group including, but not limited to, amino groups, carboxy groups, oxo groups and thiol groups, with amino groups being particularly preferred. Using these functional groups, the probes can be attached using functional groups on the probes. For example, nucleic acids containing amino groups can be attached to surfaces comprising amino groups, e.g. using linkers as are known in the art; e.g., homo-or hetero-bifunctional linkers as are well known (see 1994 Pierce Chemical Company catalog, technical section on cross-linkers, pages 155-200). In addition, in some cases, additional linkers, such as alkyl groups (including substituted and heteroalkyl groups) may be used.

In this embodiment, oligonucleotides are synthesized as is known in the art, and then attached to the surface of the solid support. As will be appreciated by those skilled in the art, either the 5′ or 3′ terminus may be attached to the solid support, or attachment may be via an internal nucleoside.

In another embodiment, the immobilization to the solid support may be very strong, yet non-covalent. For example, biotinylated oligonucleotides can be made, which bind to surfaces covalently coated with streptavidin, resulting in attachment.

Alternatively, the oligonucleotides may be synthesized on the surface, as is known in the art. For example, photoactivation techniques utilizing photopolymerization compounds and techniques are used. In a preferred embodiment, the nucleic acids can be synthesized in situ, using well known photolithographic techniques, such as those described in WO 95/25116; WO 95/35505; U.S. Pat. Nos. 5,700,637 and 5,445,934; and references cited within, all of which are expressly incorporated by reference; these methods of attachment form the basis of the Affimetrix GeneChip™ technology.

Often, amplification-based assays are performed to measure the expression level of breast cancer-associated sequences. These assays are typically performed in conjunction with reverse transcription. In such assays, a breast cancer-associated nucleic acid sequence acts as a template in an amplification reaction (e.g., Polymerase Chain Reaction, or PCR). In a quantitative amplification, the amount of amplification product will be proportional to the amount of template in the original sample. Comparison to appropriate controls provides a measure of the amount of breast cancer-associated RNA. Methods of quantitative amplification are well known to those of skill in the art. Detailed protocols for quantitative PCR are provided, e.g., in Innis et al, PCR Protocols, A Guide to Methods and Applications (1990).

In some embodiments, a TaqMan based assay is used to measure expression. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, e.g., AmpliTaq, results in the cleavage of the TaqMan probe. This cleavage separates the 5′ fluorescent dye and the 3′ quenching agent, thereby resulting in an increase in fluorescence as a function of amplification (see, e.g., literature provided by Perkin-Elmer, e.g., www2.perkin-elmer.com).

Other suitable amplification methods include, but are not limited to, ligase chain reaction (LCR) (see Wu & Wallace, Genomics 4:560 (1989), Landegren et al., Science 241:1077 (1988), and Barringer et al., Gene 89:117 (1990)), transcription amplification (Kwoh et al., Proc. Natl. Acad. Sci. USA 86:1173 (1989)), self-sustained sequence replication (Guatelli et al., Proc. Nat. Acad. Sci. USA 87:1874 (1990)), dot PCR, and linker adapter PCR, etc.

Expression of Breast Cancer Proteins from Nucleic Acids

In a preferred embodiment, breast cancer nucleic acids, e.g., encoding breast cancer proteins are used to make a variety of expression vectors to express breast cancer proteins which can then be used in screening assays, as described below. Expression vectors and recombinant DNA technology are well known to those of skill in the art (see, e.g., Ausubel, supra, and Gene Expression Systems (Fernandez & Hoeffler, eds, 1999)) and are used to express proteins. The expression vectors may be either self-replicating extrachromosomal vectors or vectors which integrate into a host genome. Generally, these expression vectors include transcriptional and translational regulatory nucleic acid operably linked to the nucleic acid encoding the breast cancer protein. The term “control sequences” refers to DNA sequences used for the expression of an operably linked coding sequence in a particular host organism. Control sequences that are suitable for prokaryotes, e.g., include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is typically accomplished by ligation at convenient restriction sites. If such sites do not exist, synthetic oligonucleotide adaptors or linkers are used in accordance with conventional practice. Transcriptional and translational regulatory nucleic acid will generally be appropriate to the host cell used to express the breast cancer protein. Numerous types of appropriate expression vectors, and suitable regulatory sequences are known in the art for a variety of host cells.

In general, transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, an expression vector may comprise additional elements. For example, the expression vector may have two replication systems, thus allowing it to be maintained in two organisms, e.g. in mammalian or insect cells for expression and in a procaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art (e.g., Fernandez & Hoeffler, supra).

In addition, in a preferred embodiment, the expression vector contains a selectable marker gene to allow the selection of transformed host cells. Selection genes are well known in the art and will vary with the host cell used.

The breast cancer proteins of the present invention are produced by culturing a host cell transformed with an expression vector containing nucleic acid encoding a breast cancer protein, under the appropriate conditions to induce or cause expression of the breast cancer protein. Conditions appropriate for breast cancer protein expression will vary with the choice of the expression vector and the host cell, and will be easily ascertained by one skilled in the art through routine experimentation or optimization. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archaebacteria, fingi, and insect and animal cells, including mammalian cells. Of particular interest are Saccharomyces cerevisiae and other yeasts, E. coli, Bacillus subtilis, Sf9 cells, C129 cells, 293 cells, Neurospora, BHK, CHO, COS, HeLa cells, HUVEC (human umbilical vein endothelial cells), THP1 cells (a macrophage cell line) and various other human cells and cell lines.

In a preferred embodiment, the breast cancer proteins are expressed in mammalian cells. Mammalian expression systems are also known in the art, and include retroviral and adenoviral systems. One expression vector system is a retroviral vector system such as is generally described in PCT/US97/01019 and PCT/US97/01048, both of which are hereby expressly incorporated by reference. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter (see, e.g., Fernandez & Hoeffler, supra). Typically, transcription termination and polyadenylation sequences recognized by mammalian cells are regulatory regions located 3′ to the translation stop codon and thus, together with the promoter elements, flank the coding sequence. Examples of transcription terminator and polyadenlyation signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, breast cancer proteins are expressed in bacterial systems. Bacterial expression systems are well known in the art. Promoters from bacteriophage may also be used and are known in the art. In addition, synthetic promoters and hybrid promoters are also useful; e.g., the tac promoter is a hybrid of the trp and lac promoter sequences. Furthermore, a bacterial promoter can include naturally occurring promoters of non-bacterial origin that have the ability to bind bacterial RNA polymerase and initiate transcription. In addition to a functioning promoter sequence, an efficient ribosome binding site is desirable. The expression vector may also include a signal peptide sequence that provides for secretion of the breast cancer protein in bacteria. The protein is either secreted into the growth media (gram-positive bacteria) or into the periplasmic space, located between the inner and outer membrane of the cell (gram-negative bacteria). The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways. These components are assembled into expression vectors. Expression vectors for bacteria are well known in the art, and include vectors for Bacillus subtilis, E. coli, Streptococcus cremoris, and Streptococcus lividans, among others (e.g., Fernandez & Hoeffler, supra). The bacterial expression vectors are transformed into bacterial host cells using techniques well known in the art, such as calcium chloride treatment, electroporation, and others.

In one embodiment, breast cancer proteins are produced in insect cells. Expression vectors for the transformation of insect cells, and in particular, baculovirus-based expression vectors, are well known in the art.

In a preferred embodiment, breast cancer protein is produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica.

The breast cancer protein may also be made as a fusion protein, using techniques well known in the art. Thus, e.g., for the creation of monoclonal antibodies, if the desired epitope is small, the breast cancer protein may be fused to a carrier protein to form an immunogen. Alternatively, the breast cancer protein may be made as a fusion protein to increase expression, or for other reasons. For example, when the breast cancer protein is a breast cancer peptide, the nucleic acid encoding the peptide may be linked to other nucleic acid for expression purposes.

In a preferred embodiment, the breast cancer protein is purified or isolated after expression. Breast cancer proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the breast cancer protein may be purified using a standard anti-breast cancer protein antibody column. Ultrafiltration and diafiltration techniques, in conjunction with protein concentration, are also useful. For general guidance in suitable purification techniques, see Scopes, Protein Purification (1982). The degree of purification necessary will vary depending on the use of the breast cancer protein. In some instances no purification will be necessary.

Once expressed and purified if necessary, the breast cancer proteins and nucleic acids are useful in a number of applications. They may be used as immunoselection reagents, as vaccine reagents, as screening agents, etc.

Variants of Breast Cancer Proteins

In one embodiment, the breast cancer proteins are derivative or variant breast cancer proteins as compared to the wild-type sequence. That is, as outlined more fully below, the derivative breast cancer peptide will often contain at least one amino acid substitution, deletion or insertion, with amino acid substitutions being particularly preferred. The amino acid substitution, insertion or deletion may occur at any residue within the breast cancer peptide.

Also included within one embodiment of breast cancer proteins of the present invention are amino acid sequence variants. These variants typically fall into one or more of three classes: substitutional, insertional or deletional variants. These variants ordinarily are prepared by site specific mutagenesis of nucleotides in the DNA encoding the breast cancer protein, using cassette or PCR mutagenesis or other techniques well known in the art, to produce DNA encoding the variant, and thereafter expressing the DNA in recombinant cell culture as outlined above. However, variant breast cancer protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the breast cancer protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed breast cancer variants screened for the optimal combination of desired activity. Techniques for making substitution mutations at predetermined sites in DNA having a known sequence are well known, e.g., M13 primer mutagenesis and PCR mutagenesis. Screening of the mutants is done using assays of breast cancer protein activities.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the breast cancer protein are desired, substitutions are generally made in accordance with the amino acid substitution relationships provided in the definition section.

The variants typically exhibit the same qualitative biological activity and will elicit the same immune response as the naturally-occurring analog, although variants also are selected to modify the characteristics of the breast cancer proteins as needed. Alternatively, the variant may be designed such that the biological activity of the breast cancer protein is altered. For example, glycosylation sites may be altered or removed.

Substantial changes in function or immunological identity are made by selecting substitutions that are less conservative than those described above. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

Covalent modifications of breast cancer polypeptides are included within the scope of this invention. One type of covalent modification includes reacting targeted amino acid residues of a breast cancer polypeptide with an organic derivatizing agent that is capable of reacting with selected side chains or the N-or C-terminal residues of a breast cancer polypeptide. Derivatization with bifunctional agents is useful, for instance, for crosslinking breast cancer polypeptides to a water-insoluble support matrix or surface for use in the method for purifying anti-breast cancer polypeptide antibodies or screening assays, as is more fully described below. Commonly used crosslinking agents include, e.g., 1,1-bis(diazoacetyl)-2-phenylethane, glutaraldehyde, N-hydroxysuccinimide esters, e.g., esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-((p-azidophenyl)dithio)propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl, threonyl or tyrosyl residues, methylation of the amino groups of the lysine, arginine, and histidine side chains (Creighton, Proteins: Structure and Molecular Properties, pp. 79-86 (1983)), acetylation of the N-terminal amine, and amidation of any C-terminal carboxyl group.

Another type of covalent modification of the breast cancer polypeptide included within the scope of this invention comprises altering the native glycosylation pattern of the polypeptide. “Altering the native glycosylation pattern” is intended for purposes herein to mean deleting one or more carbohydrate moieties found in native sequence breast cancer polypeptide, and/or adding one or more glycosylation sites that are not present in the native sequence breast cancer polypeptide. Glycosylation patterns can be altered in many ways. For example the use of different cell types to express breast cancer-associated sequences can result in different glycosylation patterns.

Addition of glycosylation sites to breast cancer polypeptides may also be accomplished by altering the amino acid sequence thereof. The alteration may be made, e.g., by the addition of, or substitution by, one or more serine or threonine residues to the native sequence breast cancer polypeptide (for O-linked glycosylation sites). The breast cancer amino acid sequence may optionally be altered through changes at the DNA level, particularly by mutating the DNA encoding the breast cancer polypeptide at preselected bases such that codons are generated that will translate into the desired amino acids.

Another means of increasing the number of carbohydrate moieties on the breast cancer polypeptide is by chemical or enzymatic coupling of glycosides to the polypeptide. Such methods are described in the art, e.g., in WO 87/05330, and in Aplin & Wriston, CRC Crit. Rev. Biochem., pp. 259-306 (1981).

Removal of carbohydrate moieties present on the breast cancer polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo-and exo-glycosidases as described by Thotakura et al., Meth. Enzymol., 138:350 (1987).

Another type of covalent modification of breast cancer comprises linking the breast cancer polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

Breast cancer polypeptides of the present invention may also be modified in a way to form chimeric molecules comprising a breast cancer polypeptide fused to another, heterologous polypeptide or amino acid sequence. In one embodiment, such a chimeric molecule comprises a fusion of a breast cancer polypeptide with a tag polypeptide which provides an epitope to which an anti-tag antibody can selectively bind. The epitope tag is generally placed at the amino-or carboxyl-terminus of the breast cancer polypeptide. The presence of such epitope-tagged forms of a breast cancer polypeptide can be detected using an antibody against the tag polypeptide. Also, provision of the epitope tag enables the breast cancer polypeptide to be readily purified by affinity purification using an anti-tag antibody or another type of affinity matrix that binds to the epitope tag. In an alternative embodiment, the chimeric molecule may comprise a fusion of a breast cancer polypeptide with an immunoglobulin or a particular region of an immunoglobulin. For a bivalent form of the chimeric molecule, such a fusion could be to the Fc region of an IgG molecule.

Various tag polypeptides and their respective antibodies are well known in the art. Examples include poly-histidine (poly-his) or poly-histidine-glycine (poly-his-gly) tags; HIS6 and metal chelation tags, the flu HA tag polypeptide and its antibody 12CA5 (Field et al., Mol. Cell. Biol. 8:2159-2165 (1988)); the c-myc tag and the 8F9, 3C7, 6E10, G4, B7 and 99E10 antibodies thereto (Evan et al., Molecular and Cellular Biology 5:3610-3616 (1985)); and the Herpes Simplex virus glycoprotein D (gD) tag and its antibody (Paborsky et al., Protein Engineering 3(6):547-553 (1990)). Other tag polypeptides include the Flag-peptide (Hopp et al., BioTechnology 6:1204-1210 (1988)); the KT3 epitope peptide (Martin et al., Science 255:192-194 (1992)); tubulin epitope peptide (Skinner et al., J. Biol. Chem. 266:15163-15166 (1991)); and the T7 gene 10 protein peptide tag (Lutz-Freyermuth et al., Proc. Natl. Acad. Sci. USA 87:6393-6397 (1990)).

Also included are other breast cancer proteins of the breast cancer family, and breast cancer proteins from other organisms, which are cloned and expressed as outlined below. Thus, probe or degenerate polymerase chain reaction (PCR) primer sequences may be used to find other related breast cancer proteins from humans or other organisms. As will be appreciated by those in the art, particularly useful probe and/or PCR primer sequences include the unique areas of the breast cancer nucleic acid sequence. As is generally known in the art, preferred PCR primers are from about 15 to about 35 nucleotides in length, with from about 20 to about 30 being preferred, and may contain inosine as needed. The conditions for the PCR reaction are well known in the art (e.g., Innis, PCR Protocols, supra).

Antibodies to Breast Cancer Proteins

In a preferred embodiment, when the breast cancer protein is to be used to generate antibodies, e.g., for immunotherapy or immunodiagnosis, the breast cancer protein should share at least one epitope or determinant with the full length protein. By “epitope” or “determinant” herein is typically meant a portion of a protein which will generate and/or bind an antibody or T-cell receptor in the context of MHC. Thus, in most instances, antibodies made to a smaller breast cancer protein will be able to bind to the full-length protein, particularly linear epitopes. In a preferred embodiment, the epitope is unique; that is, antibodies generated to a unique epitope show little or no cross-reactivity.

Methods of preparing polyclonal antibodies are known to the skilled artisan (e.g., Coligan, supra; and Harlow & Lane, supra). Polyclonal antibodies can be raised in a mammal, e.g., by one or more injections of an immunizing agent and, if desired, an adjuvant. Typically, the immunizing agent and/or adjuvant will be injected in the mammal by multiple subcutaneous or intraperitoneal injections. The immunizing agent may include a protein encoded by a nucleic acid of the figures or fragment thereof or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The antibodies may, alternatively, be monoclonal antibodies. Monoclonal antibodies may be prepared using hybridoma methods, such as those described by Kohler & Milstein, Nature 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a polypeptide encoded by a nucleic acid of Tables 1-25 or fragment thereof, or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, pp. 59-103 (1986)). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In one embodiment, the antibodies are bispecific antibodies. Bispecific antibodies are monoclonal, preferably human or humanized, antibodies that have binding specificities for at least two different antigens or that have binding specificities for two epitopes on the same antigen. In one embodiment, one of the binding specificities is for a protein encoded by a nucleic acid Tables 1-25 or a fragment thereof, the other one is for any other antigen, and preferably for a cell-surface protein or receptor or receptor subunit, preferably one that is tumor specific. Alternatively, tetramer-type technology may create multivalent reagents.

In a preferred embodiment, the antibodies to breast cancer protein are capable of reducing or eliminating a biological function of a breast cancer protein, as is described below. That is, the addition of anti-breast cancer protein antibodies (either polyclonal or preferably monoclonal) to breast cancer tissue (or cells containing breast cancer) may reduce or eliminate the breast cancer. Generally, at least a 25% decrease in activity, growth, size or the like is preferred, with at least about 50% being particularly preferred and about a 95-100% decrease being especially preferred.

In a preferred embodiment the antibodies to the breast cancer proteins are humanized antibodies (e.g., Xenerex Biosciences, Mederex, Inc., Abgenix, Inc., Protein Design Labs, Inc.) Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

Human antibodies can also be produced using various techniques known in the art, including phage display libraries (Hoogenboom & Winter, J. Mol. Biol. 227:381 (1991); Marks et al, J. Mol. Biol. 222:581 (1991)). The techniques of Cole et al. and Boemer et al. are also available for the preparation of human monoclonal antibodies (Cole et al., Monoclonal Antibodies and Cancer Therapy, p. 77 (1985) and Boerner et al., J. Immunol. 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg & Huszar, Intern. Rev. Immunol. 13:65-93 (1995).

By immunotherapy is meant treatment of breast cancer with an antibody raised against breast cancer proteins. As used herein, immunotherapy can be passive or active. Passive immunotherapy as defined herein is the passive transfer of antibody to a recipient (patient). Active immunization is the induction of antibody and/or T-cell responses in a recipient (patient). Induction of an immune response is the result of providing the recipient with an antigen to which antibodies are raised. As appreciated by one of ordinary skill in the art, the antigen may be provided by injecting a polypeptide against which antibodies are desired to be raised into a recipient, or contacting the recipient with a nucleic acid capable of expressing the antigen and under conditions for expression of the antigen, leading to an immune response.

In a preferred embodiment the breast cancer proteins against which antibodies are raised are secreted proteins as described above. Without being bound by theory, antibodies used for treatment, bind and prevent the secreted protein from binding to its receptor, thereby inactivating the secreted breast cancer protein.

In another preferred embodiment, the breast cancer protein to which antibodies are raised is a transmembrane protein. Without being bound by theory, antibodies used for treatment, bind the extracellular domain of the breast cancer protein and prevent it from binding to other proteins, such as circulating ligands or cell-associated molecules. The antibody may cause down-regulation of the transmembrane breast cancer protein. As will be appreciated by one of ordinary skill in the art, the antibody may be a competitive, non-competitive or uncompetitive inhibitor of protein binding to the extracellular domain of the breast cancer protein. The antibody is also an antagonist of the breast cancer protein. Further, the antibody prevents activation of the transmembrane breast cancer protein. In one aspect, when the antibody prevents the binding of other molecules to the breast cancer protein, the antibody prevents growth of the cell. The antibody may also be used to target or sensitize the cell to cytotoxic agents, including, but not limited to TNF-α, TNF-β, IL-1, INF-γ and IL-2, or chemotherapeutic agents including 5FU, vinblastine, actinomycin D, cisplatin, methotrexate, and the like. In some instances the antibody belongs to a sub-type that activates serum complement when complexed with the transmembrane protein thereby mediating cytotoxicity or antigen-dependent cytotoxicity (ADCC). Thus, breast cancer is treated by administering to a patient antibodies directed against the transmembrane breast cancer protein. Antibody-labeling may activate a co-toxin, localize a toxin payload, or otherwise provide means to locally ablate cells.

In another preferred embodiment, the antibody is conjugated to an effector moiety. The effector moiety can be any number of molecules, including labelling moieties such as radioactive labels or fluorescent labels, or can be a therapeutic moiety. In one aspect the therapeutic moiety is a small molecule that modulates the activity of the breast cancer protein. In another aspect the therapeutic moiety modulates the activity of molecules associated with or in close proximity to the breast cancer protein. The therapeutic moiety may inhibit enzymatic activity such as protease or collagenase or protein kinase activity associated with breast cancer.

In a preferred embodiment, the therapeutic moiety can also be a cytotoxic agent. In this method, targeting the cytotoxic agent to breast cancer tissue or cells, results in a reduction in the number of afflicted cells, thereby reducing symptoms associated with breast cancer. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin and the like. Cytotoxic agents also include radiochemicals made by conjugating radioisotopes to antibodies raised against breast cancer proteins, or binding of a radionuclide to a chelating agent that has been covalently attached to the antibody. Targeting the therapeutic moiety to transmembrane breast cancer proteins not only serves to increase the local concentration of therapeutic moiety in the breast cancer afflicted area, but also serves to reduce deleterious side effects that may be associated with the therapeutic moiety.

In another preferred embodiment, the breast cancer protein against which the antibodies are raised is an intracellular protein. In this case, the antibody may be conjugated to a protein which facilitates entry into the cell. In one case, the antibody enters the cell by endocytosis. In another embodiment, a nucleic acid encoding the antibody is administered to the individual or cell. Moreover, wherein the breast cancer protein can be targeted within a cell, i.e., the nucleus, an antibody thereto contains a signal for that target localization, i.e., a nuclear localization signal.

The breast cancer antibodies of the invention specifically bind to breast cancer proteins. By “specifically bind” herein is meant that the antibodies bind to the protein with a K_{d} of at least about 0.1 mM, more usually at least about 1 μM, preferably at least about 0.1 μM or better, and most preferably, 0.01 μM or better. Selectivity of binding is also important.

Detection of Breast Cancer Sequence for Diagnostic and Therapeutic Applications

In one aspect, the RNA expression levels of genes are determined for different cellular states in the breast cancer phenotype. Expression levels of genes in normal tissue (i.e., not undergoing breast cancer) and in breast cancer tissue (and in some cases, for varying severities of breast cancer that relate to prognosis, as outlined below) are evaluated to provide expression profiles. An expression profile of a particular cell state or point of development is essentially a “fingerprint” of the state. While two states may have any particular gene similarly expressed, the evaluation of a number of genes simultaneously allows the generation of a gene expression profile that is reflective of the state of the cell. By comparing expression profiles of cells in different states, information regarding which genes are important (including both up- and down-regulation of genes) in each of these states is obtained. Then, diagnosis may be performed or confirmed to determine whether a tissue sample has the gene expression profile of normal or cancerous tissue. This will provide for molecular diagnosis of related conditions.

“Differential expression,” or grammatical equivalents as used herein, refers to qualitative or quantitative differences in the temporal and/or cellular gene expression patterns within and among cells and tissue. Thus, a differentially expressed gene can qualitatively have its expression altered, including an activation or inactivation, in, e.g., normal versus breast cancer tissue. Genes may be turned on or turned off in a particular state, relative to another state thus permitting comparison of two or more states. A qualitatively regulated gene will exhibit an expression pattern within a state or cell type which is detectable by standard techniques. Some genes will be expressed in one state or cell type, but not in both. Alternatively, the difference in expression may be quantitative, e.g., in that expression is increased or decreased; i.e., gene expression is either upregulated, resulting in an increased amount of transcript, or downregulated, resulting in a decreased amount of transcript. The degree to which expression differs need only be large enough to quantify via standard characterization techniques as outlined below, such as by use of Affymetrix GeneChip™ expression arrays, Lockhart, Nature Biotechnology 14:1675-1680 (1996), hereby expressly incorporated by reference. Other techniques include, but are not limited to, quantitative reverse transcriptase PCR, northern analysis and RNase protection. As outlined above, preferably the change in expression (i.e., upregulation or downregulation) is at least about 50%, more preferably at least about 100%, more preferably at least about 150%, more preferably at least about 200%, with from 300 to at least 1000% being especially preferred.

Evaluation may be at the gene transcript, or the protein level. The amount of gene expression may be monitored using nucleic acid probes to the DNA or RNA equivalent of the gene transcript, and the quantification of gene expression levels, or, alternatively, the final gene product itself (protein) can be monitored, e.g., with antibodies to the breast cancer protein and standard immunoassays (ELISAs, etc.) or other techniques, including mass spectroscopy assays, 2D gel electrophoresis assays, etc. Proteins corresponding to breast cancer genes, i.e., those identified as being important in a breast cancer phenotype, can be evaluated in a breast cancer diagnostic test.

In a preferred embodiment, gene expression monitoring is performed simultaneously on a number of genes. Multiple protein expression monitoring can be performed as well. Similarly, these assays may be performed on an individual basis as well.

In this embodiment, the breast cancer nucleic acid probes are attached to biochips as outlined herein for the detection and quantification of breast cancer sequences in a particular cell. The assays are further described below in the example. PCR techniques can be used to provide greater sensitivity.

In a preferred embodiment nucleic acids encoding the breast cancer protein are detected. Although DNA or RNA encoding the breast cancer protein may be detected, of particular interest are methods wherein an mRNA encoding a breast cancer protein is detected. Probes to detect mRNA can be a nucleotide/deoxynucleotide probe that is complementary to and hybridizes with the mRNA and includes, but is not limited to, oligonucleotides, cDNA or RNA. Probes also should contain a detectable label, as defined herein. In one method the mRNA is detected after immobilizing the nucleic acid to be examined on a solid support such as nylon membranes and hybridizing the probe with the sample. Following washing to remove the non-specifically bound probe, the label is detected. In another method detection of the mRNA is performed in situ. In this method permeabilized cells or tissue samples are contacted with a detectably labeled nucleic acid probe for sufficient time to allow the probe to hybridize with the target mRNA. Following washing to remove the non-specifically bound probe, the label is detected. For example a digoxygenin labeled riboprobe (RNA probe) that is complementary to the mRNA encoding a breast cancer protein is detected by binding the digoxygenin with an anti-digoxygenin secondary antibody and developed with nitro blue tetrazolium and 5-bromo-4-chloro-3-indoyl phosphate.

In a preferred embodiment, various proteins from the three classes of proteins as described herein (secreted, transmembrane or intracellular proteins) are used in diagnostic assays. The breast cancer proteins, antibodies, nucleic acids, modified proteins and cells containing breast cancer sequences are used in diagnostic assays. This can be performed on an individual gene or corresponding polypeptide level. In a preferred embodiment, the expression profiles are used, preferably in conjunction with high throughput screening techniques to allow monitoring for expression profile genes and/or corresponding polypeptides.

As described and defined herein, breast cancer proteins, including intracellular, transmembrane or secreted proteins, find use as markers of breast cancer. Detection of these proteins in putative breast cancer tissue allows for detection or diagnosis of breast cancer. In one embodiment, antibodies are used to detect breast cancer proteins. A preferred method separates proteins from a sample by electrophoresis on a gel (typically a denaturing and reducing protein gel, but may be another type of gel, including isoelectric focusing gels and the like). Following separation of proteins, the breast cancer protein is detected, e.g., by immunoblotting with antibodies raised against the breast cancer protein. Methods of immunoblotting are well known to those of ordinary skill in the art.

In another preferred method, antibodies to the breast cancer protein find use in in situ imaging techniques, e.g., in histology (e.g., Methods in Cell Biology: Antibodies in Cell Biology, volume 37 (Asai, ed. 1993)). In this method cells are contacted with from one to many antibodies to the breast cancer protein(s). Following washing to remove non-specific antibody binding, the presence of the antibody or antibodies is detected. In one embodiment the antibody is detected by incubating with a secondary antibody that contains a detectable label. In another method the primary antibody to the breast cancer protein(s) contains a detectable label, e.g. an enzyme marker that can act on a substrate. In another preferred embodiment each one of multiple primary antibodies contains a distinct and detectable label. This method finds particular use in simultaneous screening for a plurality of breast cancer proteins. As will be appreciated by one of ordinary skill in the art, many other histological imaging techniques are also provided by the invention.

In a preferred embodiment the label is detected in a fluorometer which has the ability to detect and distinguish emissions of different wavelengths. In addition, a fluorescence activated cell sorter (FACS) can be used in the method.

In another preferred embodiment, antibodies find use in diagnosing breast cancer from blood, serum, plasma, stool, and other samples. Such samples, therefore, are useful as samples to be probed or tested for the presence of breast cancer proteins. Antibodies can be used to detect a breast cancer protein by previously described immunoassay techniques including ELISA, immunoblotting (western blotting), immunoprecipitation, BIACORE technology and the like. Conversely, the presence of antibodies may indicate an immune response against an endogenous breast cancer protein.

In a preferred embodiment, in situ hybridization of labeled breast cancer nucleic acid probes to tissue arrays is done. For example, arrays of tissue samples, including breast cancer tissue and/or normal tissue, are made. In situ hybridization (see, e.g., Ausubel, supra) is then performed. When comparing the fingerprints between an individual and a standard, the skilled artisan can make a diagnosis, a prognosis, or a prediction based on the findings. It is further understood that the genes which indicate the diagnosis may differ from those which indicate the prognosis and molecular profiling of the condition of the cells may lead to distinctions between responsive or refractory conditions or may be predictive of outcomes.

In a preferred embodiment, the breast cancer proteins, antibodies, nucleic acids, modified proteins and cells containing breast cancer sequences are used in prognosis assays. As above, gene expression profiles can be generated that correlate to breast cancer, in terms of long term prognosis. Again, this may be done on either a protein or gene level, with the use of genes being preferred. As above, breast cancer probes may be attached to biochips for the detection and quantification of breast cancer sequences in a tissue or patient. The assays proceed as outlined above for diagnosis. PCR method may provide more sensitive and accurate quantification.

Assays for Therapeutic Compounds

In a preferred embodiment members of the proteins, nucleic acids, and antibodies as described herein are used in drug screening assays. The breast cancer proteins, antibodies, nucleic acids, modified proteins and cells containing breast cancer sequences are used in drug screening assays or by evaluating the effect of drug candidates on a “gene expression profile” or expression profile of polypeptides. In a preferred embodiment, the expression profiles are used, preferably in conjunction with high throughput screening techniques to allow monitoring for expression profile genes after treatment with a candidate agent (e.g., Zlokarnik, et al., Science 279:84-8 (1998); Heid, Genome Res 6:986-94, 1996).

In a preferred embodiment, the breast cancer proteins, antibodies, nucleic acids, modified proteins and cells containing the native or modified breast cancer proteins are used in screening assays. That is, the present invention provides novel methods for screening for compositions which modulate the breast cancer phenotype or an identified physiological function of a breast cancer protein. As above, this can be done on an individual gene level or by evaluating the effect of drug candidates on a “gene expression profile”. In a preferred embodiment, the expression profiles are used, preferably in conjunction with high throughput screening techniques to allow monitoring for expression profile genes after treatment with a candidate agent, see Zlokarnik, supra.

Having identified the differentially expressed genes herein, a variety of assays may be executed. In a preferred embodiment, assays may be run on an individual gene or protein level. That is, having identified a particular gene as up regulated in breast cancer, test compounds can be screened for the ability to modulate gene expression or for binding to the breast cancer protein. “Modulation” thus includes both an increase and a decrease in gene expression. The preferred amount of modulation will depend on the original change of the gene expression in normal versus tissue undergoing breast cancer, with changes of at least 10%, preferably 50%, more preferably 100-300%, and in some embodiments 300-1000% or greater. Thus, if a gene exhibits a 4-fold increase in breast cancer tissue compared to normal tissue, a decrease of about four-fold is often desired; similarly, a 10-fold decrease in breast cancer tissue compared to normal tissue often provides a target value of a 10-fold increase in expression to be induced by the test compound.

The amount of gene expression may be monitored using nucleic acid probes and the quantification of gene expression levels, or, alternatively, the gene product itself can be monitored, e.g., through the use of antibodies to the breast cancer protein and standard immunoassays. Proteomics and separation techniques may also allow quantification of expression.

In a preferred embodiment, gene expression or protein monitoring of a number of entities, i.e., an expression profile, is monitored simultaneously. Such profiles will typically involve a plurality of those entities described herein.

In this embodiment, the breast cancer nucleic acid probes are attached to biochips as outlined herein for the detection and quantification of breast cancer sequences in a particular cell. Alternatively, PCR may be used. Thus, a series, e.g., of microtiter plate, may be used with dispensed primers in desired wells. A PCR reaction can then be performed and analyzed for each well.

Expression monitoring can be performed to identify compounds that modify the expression of one or more breast cancer-associated sequences, e.g., a polynucleotide sequence set out in Table 17. Generally, in a preferred embodiment, a test modulator is added to the cells prior to analysis. Moreover, screens are also provided to identify agents that modulate breast cancer, modulate breast cancer proteins, bind to a breast cancer protein, or interfere with the binding of a breast cancer protein and an antibody or other binding partner.

The term “test compound” or “drug candidate” or “modulator” or grammatical equivalents as used herein describes any molecule, e.g., protein, oligopeptide, small organic molecule, polysaccharide, polynucleotide, etc., to be tested for the capacity to directly or indirectly alter the breast cancer phenotype or the expression of a breast cancer sequence, e.g., a nucleic acid or protein sequence. In preferred embodiments, modulators alter expression profiles, or expression profile nucleic acids or proteins provided herein. In one embodiment, the modulator suppresses a breast cancer phenotype, e.g. to a normal tissue fingerprint. In another embodiment, a modulator induced a breast cancer phenotype. Generally, a plurality of assay mixtures are run in parallel with different agent concentrations to obtain a differential response to the various concentrations. Typically, one of these concentrations serves as a negative control, i.e., at zero concentration or below the level of detection.

Drug candidates encompass numerous chemical classes, though typically they are organic molecules, preferably small organic compounds having a molecular weight of more than 100 and less than about 2,500 daltons. Preferred small molecules are less than 2000, or less than 1500 or less than 1000 or less than 500 D. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

In one aspect, a modulator will neutralize the effect of a breast cancer protein. By “neutralize” is meant that activity of a protein is inhibited or blocked and the consequent effect on the cell.

In certain embodiments, combinatorial libraries of potential modulators will be screened for an ability to bind to a breast cancer polypeptide or to modulate activity. Conventionally, new chemical entities with useful properties are generated by identifying a chemical compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

In one preferred embodiment, high throughput screening methods involve providing a library containing a large number of potential therapeutic compounds (candidate compounds). Such “combinatorial chemical libraries” are then screened in one or more assays to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library, such as a polypeptide (e.g., mutein) library, is formed by combining a set of chemical building blocks called amino acids in every possible way for a given compound length (i.e., the number of amino acids in a polypeptide compound). Millions of chemical compounds can be synthesized through such combinatorial mixing of chemical building blocks (Gallop et al., J. Med. Chem. 37(9):1233-1251 (1994)).

Preparation and screening of combinatorial chemical libraries is well known to those of skill in the art. Such combinatorial chemical libraries include, but are not limited to, peptide libraries (see, e.g., U.S. Pat. No. 5,010,175, Furka, Pept. Prot. Res. 37:487-493 (1991), Houghton et al., Nature, 354:84-88 (1991)), peptoids (PCT Publication No WO 91/19735), encoded peptides (PCT Publication WO 93/20242), random bio-oligomers (PCT Publication WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with a Beta-D-Glucose scaffolding (Hirschmann et al, J. Amer. Chem. Soc. 114:9217-9218 (1992)), analogous organic syntheses of small compound libraries (Chen et al., J. Amer. Chem. Soc. 116:2661 (1994)), oligocarbamates (Cho, et al., Science 261:1303 (1993)), and/or peptidyl phosphonates (Campbell et al., J. Org. Chem. 59:658 (1994)). See, generally, Gordon et al., J. Med. Chem. 37:1385 (1994), nucleic acid libraries (see, e.g., Strategene, Corp.), peptide nucleic acid libraries (see, e.g., U.S. Pat. No. 5,539,083), antibody libraries (see, e.g., Vaughn et al., Nature Biotechnology 14(3):309-314 (1996), and PCT/US96/10287), carbohydrate libraries (see, e.g. Liang et al., Science 274:1520-1522 (1996), and U.S. Pat. No. 5,593,853), and small organic molecule libraries (see, e.g., benzodiazepines, Baum, C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514; and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.), which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

The assays to identify modulators are amenable to high throughput screening. Preferred assays thus detect enhancement or inhibition of breast cancer gene transcription, inhibition or enhancement of polypeptide expression, and inhibition or enhancement of polypeptide activity.

High throughput assays for the presence, absence, quantification, or other properties of particular nucleic acids or protein products are well known to those of skill in the art. Similarly, binding assays and reporter gene assays are similarly well known. Thus, e.g., U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures, including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, e.g., Zymark Corp. provides technical bulletins describing screening systems for detecting the modulation of gene transcription, ligand binding, and the like.

In one embodiment, modulators are proteins, often naturally occurring proteins or fragments of naturally occurring proteins. Thus, e.g., cellular extracts containing proteins, or random or directed digests of proteinaceous cellular extracts, may be used. In this way libraries of proteins may be made for screening in the methods of the invention. Particularly preferred in this embodiment are libraries of bacterial, fungal, viral, and mammalian proteins, with the latter being preferred, and human proteins being especially preferred. Particularly useful test compound will be directed to the class of proteins to which the target belongs, e.g., substrates for enzymes or ligands and receptors.

In a preferred embodiment, modulators are peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, e.g., of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc., or to purines, etc.

Modulators of breast cancer can also be nucleic acids, as defined above.

As described above generally for proteins, nucleic acid modulating agents may be naturally occurring nucleic acids, random nucleic acids, or “biased” random nucleic acids. For example, digests of procaryotic or eucaryotic genomes may be used as is outlined above for proteins.

In a preferred embodiment, the candidate compounds are organic chemical moieties, a wide variety of which are available in the literature.

After the candidate agent has been added and the cells allowed to incubate for some period of time, the sample containing a target sequence to be analyzed is added to the biochip. If required, the target sequence is prepared using known techniques. For example, the sample may be treated to lyse the cells, using known lysis buffers, electroporation, etc., with purification and/or amplification such as PCR performed as appropriate. For example, an in vitro transcription with labels covalently attached to the nucleotides is performed. Generally, the nucleic acids are labeled with biotin-FITC or PE, or with cy3 or cy5.

In a preferred embodiment, the target sequence is labeled with, e.g., a fluorescent, a chemiluminescent, a chemical, or a radioactive signal, to provide a means of detecting the target sequence's specific binding to a probe. The label also can be an enzyme, such as, alkaline phosphatase or horseradish peroxidase, which when provided with an appropriate substrate produces a product that can be detected. Alternatively, the label can be a labeled compound or small molecule, such as an enzyme inhibitor, that binds but is not catalyzed or altered by the enzyme. The label also can be a moiety or compound, such as, an epitope tag or biotin which specifically binds to streptavidin. For the example of biotin, the streptavidin is labeled as described above, thereby, providing a detectable signal for the bound target sequence. Unbound labeled streptavidin is typically removed prior to analysis.

As will be appreciated by those in the art, these assays can be direct hybridization assays or can comprise “sandwich assays”, which include the use of multiple probes, as is generally outlined in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. In this embodiment, in general, the target nucleic acid is prepared as outlined above, and then added to the biochip comprising a plurality of nucleic acid probes, under conditions that allow the formation of a hybridization complex.

A variety of hybridization conditions may be used in the present invention, including high, moderate and low stringency conditions as outlined above. The assays are generally run under stringency conditions which allows formation of the label probe hybridization complex only in the presence of target. Stringency can be controlled by altering a step parameter that is a thermodynamic variable, including, but not limited to, temperature, formamide concentration, salt concentration, chaotropic salt concentration pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

The reactions outlined herein may be accomplished in a variety of ways. Components of the reaction may be added simultaneously, or sequentially, in different orders, with preferred embodiments outlined below. In addition, the reaction may include a variety of other reagents. These include salts, buffers, neutral proteins, e.g. albumin, detergents, etc. which may be used to facilitate optimal hybridization and detection, and/or reduce non-specific or background interactions. Reagents that otherwise improve the efficiency of the assay, such as protease inhibitors, nuclease inhibitors, anti-microbial agents, etc., may also be used as appropriate, depending on the sample preparation methods and purity of the target.

The assay data are analyzed to determine the expression levels, and changes in expression levels as between states, of individual genes, forming a gene expression profile.

Screens are performed to identify modulators of the breast cancer phenotype. In one embodiment, screening is performed to identify modulators that can induce or suppress a particular expression profile, thus preferably generating the associated phenotype. In another embodiment, e.g., for diagnostic applications, having identified differentially expressed genes important in a particular state, screens can be performed to identify modulators that alter expression of individual genes. In an another embodiment, screening is performed to identify modulators that alter a biological function of the expression product of a differentially expressed gene. Again, having identified the importance of a gene in a particular state, screens are performed to identify agents that bind and/or modulate the biological activity of the gene product.

In addition screens can be done for genes that are induced in response to a candidate agent. After identifying a modulator based upon its ability to suppress a breast cancer expression pattern leading to a normal expression pattern, or to modulate a single breast cancer gene expression profile so as to mimic the expression of the gene from normal tissue, a screen as described above can be performed to identify genes that are specifically modulated in response to the agent. Comparing expression profiles between normal tissue and agent treated breast cancer tissue reveals genes that are not expressed in normal tissue or breast cancer tissue, but are expressed in agent treated tissue. These agent-specific sequences can be identified and used by methods described herein for breast cancer genes or proteins. In particular these sequences and the proteins they encode find use in marking or identifying agent treated cells. In addition, antibodies can be raised against the agent induced proteins and used to target novel therapeutics to the treated breast cancer tissue sample.

Thus, in one embodiment, a test compound is administered to a population of breast cancer cells, that have an associated breast cancer expression profile. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e., a peptide) may be put into a viral construct such as an adenoviral or retroviral construct, and added to the cell, such that expression of the peptide agent is accomplished, e.g., PCT US97/01019. Regulatable gene therapy systems can also be used.

Once the test compound has been administered to the cells, the cells can be washed if desired and are allowed to incubate under preferably physiological conditions for some period of time. The cells are then harvested and a new gene expression profile is generated, as outlined herein.

Thus, e.g., breast cancer tissue may be screened for agents that modulate, e.g., induce or suppress the breast cancer phenotype. A change in at least one gene, preferably many, of the expression profile indicates that the agent has an effect on breast cancer activity. By defining such a signature for the breast cancer phenotype, screens for new drugs that alter the phenotype can be devised. With this approach, the drug target need not be known and need not be represented in the original expression screening platform, nor does the level of transcript for the target protein need to change.

In a preferred embodiment, as outlined above, screens may be done on individual genes and gene products (proteins). That is, having identified a particular differentially expressed gene as important in a particular state, screening of modulators of either the expression of the gene or the gene product itself can be done. The gene products of differentially expressed genes are sometimes referred to herein as “breast cancer proteins” or a “breast cancer modulatory protein”. The breast cancer modulatory protein may be a fragment, or alternatively, be the full length protein to the fragment encoded by the nucleic acids of the Tables. Preferably, the breast cancer modulatory protein is a fragment. In a preferred embodiment, the breast cancer amino acid sequence which is used to determine sequence identity or similarity is encoded by a nucleic acid of Table 25. In another embodiment, the sequences are naturally occurring allelic variants of a protein encoded by a nucleic acid of Table 25. In another embodiment, the sequences are sequence variants as further described herein.

Preferably, the breast cancer modulatory protein is a fragment of approximately 14 to 24 amino acids long. More preferably the fragment is a soluble fragment. Preferably, the fragment includes a non-transmembrane region. In a preferred embodiment, the fragment has an N-terminal Cys to aid in solubility. In one embodiment, the C-terminus of the fragment is kept as a free acid and the N-terminus is a free amine to aid in coupling, i.e., to cysteine.

In one embodiment the breast cancer proteins are conjugated to an immunogenic agent as discussed herein. In one embodiment the breast cancer protein is conjugated to BSA.

Measurements of breast cancer polypeptide activity, or of breast cancer or the breast cancer phenotype can be performed using a variety of assays. For example, the effects of the test compounds upon the function of the breast cancer polypeptides can be measured by examining parameters described above. A suitable physiological change that affects activity can be used to assess the influence of a test compound on the polypeptides of this invention. When the functional consequences are determined using intact cells or animals, one can also measure a variety of effects such as, in the case of breast cancer associated with tumors, tumor growth, tumor metastasis, neovascularization, hormone release, transcriptional changes to both known and uncharacterized genetic markers (e.g., northern blots), changes in cell metabolism such as cell growth or pH changes, and changes in intracellular second messengers such as cGMP. In the assays of the invention, mammalian breast cancer polypeptide is typically used, e.g., mouse, preferably human.

Assays to identify compounds with modulating activity can be performed in vitro. For example, a breast cancer polypeptide is first contacted with a potential modulator and incubated for a suitable amount of time, e.g., from 0.5 to 48 hours. In one embodiment, the breast cancer polypeptide levels are determined in vitro by measuring the level of protein or mRNA. The level of protein is measured using immunoassays such as western blotting, ELISA and the like with an antibody that selectively binds to the breast cancer polypeptide or a fragment thereof. For measurement of mRNA, amplification, e.g., using PCR, LCR, or hybridization assays, e.g., northern hybridization, RNAse protection, dot blotting, are preferred. The level of protein or mRNA is detected using directly or indirectly labeled detection agents, e.g., fluorescently or radioactively labeled nucleic acids, radioactively or a enzymatically labeled antibodies, and the like, as described herein.

Alternatively, a reporter gene system can be devised using the breast cancer protein promoter operably linked to a reporter gene such as luciferase, green fluorescent protein, CAT, or β-gal. The reporter construct is typically transfected into a cell. After treatment with a potential modulator, the amount of reporter gene transcription, translation, or activity is measured according to standard techniques known to those of skill in the art.

In a preferred embodiment, as outlined above, screens may be done on individual genes and gene products (proteins). That is, having identified a particular differentially expressed gene as important in a particular state, screening of modulators of the expression of the gene or the gene product itself can be done. The gene products of differentially expressed genes are sometimes referred to herein as “breast cancer proteins.” The breast cancer protein may be a fragment, or alternatively, be the full length protein to a fragment shown herein.

In one embodiment, screening for modulators of expression of specific genes is performed. Typically, the expression of only one or a few genes are evaluated. In another embodiment, screens are designed to first find compounds that bind to differentially expressed proteins. These compounds are then evaluated for the ability to modulate differentially expressed activity. Moreover, once initial candidate compounds are identified, variants can be farther screened to better evaluate structure activity relationships.

In a preferred embodiment, binding assays are done. In general, purified or isolated gene product is used; that is, the gene products of one or more differentially expressed nucleic acids are made. For example, antibodies are generated to the protein gene products, and standard immunoassays are run to determine the amount of protein present. Alternatively, cells comprising the breast cancer proteins can be used in the assays.

Thus, in a preferred embodiment, the methods comprise combining a breast cancer protein and a candidate compound, and determining the binding of the compound to the breast cancer protein. Preferred embodiments utilize the human breast cancer protein, although other mammalian proteins may also be used, e.g. for the development of animal models of human disease. In some embodiments, as outlined herein, variant or derivative breast cancer proteins may be used.

Generally, in a preferred embodiment of the methods herein, the breast cancer protein or the candidate agent is non-diffusably bound to an insoluble support having isolated sample receiving areas (e.g. a microtiter plate, an array, etc.). The insoluble supports may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, teflon™, etc. Microtiter plates and arrays are especially convenient because a large number of assays can be carried out simultaneously, using small amounts of reagents and samples. The particular manner of binding of the composition is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

In a preferred embodiment, the breast cancer protein is bound to the support, and a test compound is added to the assay. Alternatively, the candidate agent is bound to the support and the breast cancer protein is added. Novel binding agents include specific antibodies, non-natural binding agents identified in screens of chemical libraries, peptide analogs, etc. Of particular interest are screening assays for agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the test modulating compound to the breast cancer protein may be done in a number of ways. In a preferred embodiment, the compound is labeled, and binding determined directly, e.g., by attaching all or a portion of the breast cancer protein to a solid support, adding a labeled candidate agent (e.g., a fluorescent label), washing off excess reagent, and determining whether the label is present on the solid support. Various blocking and washing steps may be utilized as appropriate.

In some embodiments, only one of the components is labeled, e.g., the proteins (or proteinaceous candidate compounds) can be labeled. Alternatively, more than one component can be labeled with different labels, e.g., ¹²⁵I for the proteins and a fluorophor for the compound. Proximity reagents, e.g., quenching or energy transfer reagents are also useful.

In one embodiment, the binding of the test compound is determined by competitive binding assay. The competitor is a binding moiety known to bind to the target molecule (i.e., a breast cancer protein), such as an antibody, peptide, binding partner, ligand, etc. Under certain circumstances, there may be competitive binding between the compound and the binding moiety, with the binding moiety displacing the compound. In one embodiment, the test compound is labeled. Either the compound, or the competitor, or both, is added first to the protein for a time sufficient to allow binding, if present. Incubations may be performed at a temperature which facilitates optimal activity, typically between 4 and 40° C. Incubation periods are typically optimized, e.g., to facilitate rapid high throughput screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the test compound. Displacement of the competitor is an indication that the test compound is binding to the breast cancer protein and thus is capable of binding to, and potentially modulating, the activity of the breast cancer protein. In this embodiment, either component can be labeled. Thus, e.g., if the competitor is labeled, the presence of label in the wash solution indicates displacement by the agent. Alternatively, if the test compound is labeled, the presence of the label on the support indicates displacement.

In an alternative embodiment, the test compound is added first, with incubation and washing, followed by the competitor. The absence of binding by the competitor may indicate that the test compound is bound to the breast cancer protein with a higher affinity. Thus, if the test compound is labeled, the presence of the label on the support, coupled with a lack of competitor binding, may indicate that the test compound is capable of binding to the breast cancer protein.

In a preferred embodiment, the methods comprise differential screening to identity agents that are capable of modulating the activity of the breast cancer proteins. In this embodiment, the methods comprise combining a breast cancer protein and a competitor in a first sample. A second sample comprises a test compound, a breast cancer protein, and a competitor. The binding of the competitor is determined for both samples, and a change, or difference in binding between the two samples indicates the presence of an agent capable of binding to the breast cancer protein and potentially modulating its activity. That is, if the binding of the competitor is different in the second sample relative to the first sample, the agent is capable of binding to the breast cancer protein.

Alternatively, differential screening is used to identify drug candidates that bind to the native breast cancer protein, but cannot bind to modified breast cancer proteins. The structure of the breast cancer protein may be modeled, and used in rational drug design to synthesize agents that interact with that site. Drug candidates that affect the activity of a breast cancer protein are also identified by screening drugs for the ability to either enhance or reduce the activity of the protein.

Positive controls and negative controls may be used in the assays. Preferably control and test samples are performed in at least triplicate to obtain statistically significant results. Incubation of all samples is for a time sufficient for the binding of the agent to the protein. Following incubation, samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g. albumin, detergents, etc. which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in an order that provides for the requisite binding.

In a preferred embodiment, the invention provides methods for screening for a compound capable of modulating the activity of a breast cancer protein. The methods comprise adding a test compound, as defined above, to a cell comprising breast cancer proteins. Preferred cell types include almost any cell. The cells contain a recombinant nucleic acid that encodes a breast cancer protein. In a preferred embodiment, a library of candidate agents are tested on a plurality of cells.

In one aspect, the assays are evaluated in the presence or absence or previous or subsequent exposure of physiological signals, e.g. hormones, antibodies, peptides, antigens, cytokines, growth factors, action potentials, pharmacological agents including chemotherapeutics, radiation, carcinogenics, or other cells (i.e. cell-cell contacts). In another example, the determinations are determined at different stages of the cell cycle process.

In this way, compounds that modulate breast cancer agents are identified. Compounds with pharmacological activity are able to enhance or interfere with the activity of the breast cancer protein. Once identified, similar structures are evaluated to identify critical structural feature of the compound.

In one embodiment, a method of inhibiting breast cancer cell division is provided. The method comprises administration of a breast cancer inhibitor. In another embodiment, a method of inhibiting breast cancer is provided. The method comprises administration of a breast cancer inhibitor. In a further embodiment, methods of treating cells or individuals with breast cancer are provided. The method comprises administration of a breast cancer inhibitor.

In one embodiment, a breast cancer inhibitor is an antibody as discussed above. In another embodiment, the breast cancer inhibitor is an antisense molecule.

A variety of cell growth, proliferation, and metastasis assays are known to those of skill in the art, as described below.

Soft Agar Growth or Colony Formation in Suspension

Normal cells require a solid substrate to attach and grow. When the cells are transformed, they lose this phenotype and grow detached from the substrate. For example, transformed cells can grow in stirred suspension culture or suspended in semi-solid media, such as semi-solid or soft agar. The transformed cells, when transfected with tumor suppressor genes, regenerate normal phenotype and require a solid substrate to attach and grow. Soft agar growth or colony formation in suspension assays can be used to identify modulators of breast cancer sequences, which when expressed in host cells, inhibit abnormal cellular proliferation and transformation. A therapeutic compound would reduce or eliminate the host cells' ability to grow in stirred suspension culture or suspended in semi-solid media, such as semi-solid or soft.

Techniques for soft agar growth or colony formation in suspension assays are described in Freshney, Culture of Animal Cells a Manual of Basic Technique (3^{rd} ed., 1994), herein incorporated by reference. See also, the methods section of Garkavtsev et al. (1996), supra, herein incorporated by reference.

Contact Inhibition and Density Limitation of Growth

Normal cells typically grow in a flat and organized pattern in a petri dish until they touch other cells. When the cells touch one another, they are contact inhibited and stop growing. When cells are transformed, however, the cells are not contact inhibited and continue to grow to high densities in disorganized foci. Thus, the transformed cells grow to a higher saturation density than normal cells. This can be detected morphologically by the formation of a disoriented monolayer of cells or rounded cells in foci within the regular pattern of normal surrounding cells. Alternatively, labeling index with (³H)-thymidine at saturation density can be used to measure density limitation of growth. See Freshney (1994), supra. The transformed cells, when transfected with tumor suppressor genes, regenerate a normal phenotype and become contact inhibited and would grow to a lower density.

In this assay, labeling index with (³H)-thymidine at saturation density is a preferred method of measuring density limitation of growth. Transformed host cells are transfected with a breast cancer-associated sequence and are grown for 24 hours at saturation density in non-limiting medium conditions. The percentage of cells labeling with (3H)-thymidine is determined autoradiographically. See, Freshney (1994), supra.

Growth Factor or Serum Dependence

Transformed cells have a lower serum dependence than their normal counterparts (see, e.g., Temin, J. Natl. Cancer Insti. 37:167-175 (1966); Eagle et al., J. Exp. Med. 131:836-879 (1970)); Freshney, supra. This is in part due to release of various growth factors by the transformed cells. Growth factor or serum dependence of transformed host cells can be compared with that of control.

Tumor Specific Markers Levels

Tumor cells release an increased amount of certain factors (hereinafter “tumor specific markers”) than their normal counterparts. For example, plasminogen activator (PA) is released from human glioma at a higher level than from normal brain cells (see, e.g., Gullino, Angiogenesis, tumor vascularization, and potential interference with tumor growth. in Biological Responses in Cancer, pp. 178-184 (Mihich (ed.) 1985)). Similarly, Tumor angiogenesis factor (TAF) is released at a higher level in tumor cells than their normal counterparts. See, e.g., Folkman, Angiogenesis and Cancer, Sem Cancer Biol. (1992)).

Various techniques which measure the release of these factors are described in Freshney (1994), supra. Also, see, Unkless et al., J. Biol. Chem. 249:4295-4305 (1974); Strickland & Beers, J. Biol. Chem. 251:5694-5702 (1976); Whur et al., Br. J. Cancer 42:305-312 (1980); Gullino, Angiogenesis, tumor vascularization, and potential interference with tumor growth. in Biological Responses in Cancer, pp. 178-184 (Mihich (ed.) 1985); Freshney Anticancer Res. 5:111-130 (1985).

Invasiveness into Matrigel

The degree of invasiveness into Matrigel or some other extracellular matrix constituent can be used as an assay to identify compounds that modulate breast cancer-associated sequences. Tumor cells exhibit a good correlation between malignancy and invasiveness of cells into Matrigel or some other extracellular matrix constituent. In this assay, tumorigenic cells are typically used as host cells. Expression of a tumor suppressor gene in these host cells would decrease invasiveness of the host cells.

Techniques described in Freshney (1994), supra, can be used. Briefly, the level of invasion of host cells can be measured by using filters coated with Matrigel or some other extracellular matrix constituent. Penetration into the gel, or through to the distal side of the filter, is rated as invasiveness, and rated histologically by number of cells and distance moved, or by prelabeling the cells with ¹²⁵I and counting the radioactivity on the distal side of the filter or bottom of the dish. See, e.g., Freshney (1984), supra.

Tumor Growth In Vivo

Effects of breast cancer-associated sequences on cell growth can be tested in transgenic or immune-suppressed mice. Knock-out transgenic mice can be made, in which the breast cancer gene is disrupted or in which a breast cancer gene is inserted. Knock-out transgenic mice can be made by insertion of a marker gene or other heterologous gene into the endogenous breast cancer gene site in the mouse genome via homologous recombination. Such mice can also be made by substituting the endogenous breast cancer gene with a mutated version of the breast cancer gene, or by mutating the endogenous breast cancer gene, e.g., by exposure to carcinogens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C., (1987).

Alternatively, various immune-suppressed or immune-deficient host animals can be used. For example, genetically athymic “nude” mouse (see, e.g., Giovanella et al., J. Natl. Cancer Inst. 52:921 (1974)), a SCID mouse, a thymectomized mouse, or an irradiated mouse (see, e.g., Bradley et al., Br. J. Cancer 38:263 (1978); Selby et al., Br. J. Cancer 41:52 (1980)) can be used as a host. Transplantable tumor cells (typically about 106 cells) injected into isogenic hosts will produce invasive tumors in a high proportions of cases, while normal cells of similar origin will not. In hosts which developed invasive tumors, cells expressing a breast cancer-associated sequences are injected subcutaneously. After a suitable length of time, preferably 4-8 weeks, tumor growth is measured (e.g., by volume or by its two largest dimensions) and compared to the control. Tumors that have statistically significant reduction (using, e.g., Student's T test) are said to have inhibited growth.

Polynucleotide Modulators of Breast Cancer

Antisense Polynucleotides

In certain embodiments, the activity of a breast cancer-associated protein is down-regulated, or entirely inhibited, by the use of antisense polynucleotide, i.e., a nucleic acid complementary to, and which can preferably hybridize specifically to, a coding mRNA nucleic acid sequence, e.g., a breast cancer protein mRNA, or a subsequence thereof. Binding of the antisense polynucleotide to the mRNA reduces the translation and/or stability of the mRNA.

In the context of this invention, antisense polynucleotides can comprise naturally-occurring nucleotides, or synthetic species formed from naturally-occurring subunits or their close homologs. Antisense polynucleotides may also have altered sugar moieties or inter-sugar linkages. Exemplary among these are the phosphorothioate and other sulfur containing species which are known for use in the art. Analogs are comprehended by this invention so long as they function effectively to hybridize with the breast cancer protein mRNA. See, e.g., Isis Pharmaceuticals, Carlsbad, Calif.; Sequitor, Inc., Natick, Mass.

Such antisense polynucleotides can readily be synthesized using recombinant means, or can be synthesized in vitro. Equipment for such synthesis is sold by several vendors, including Applied Biosystems. The preparation of other oligonucleotides such as phosphorothioates and alkylated derivatives is also well known to those of skill in the art.

Antisense molecules as used herein include antisense or sense oligonucleotides. Sense oligonucleotides can, e.g., be employed to block transcription by binding to the anti-sense strand. The antisense and sense oligonucleotide comprise a single-stranded nucleic acid sequence (either RNA or DNA) capable of binding to target mRNA (sense) or DNA (antisense) sequences for breast cancer molecules. A preferred antisense molecule is for a breast cancer sequences in Tables 1-25, or for a ligand or activator thereof. Antisense or sense oligonucleotides, according to the present invention, comprise a fragment generally at least about 14 nucleotides, preferably from about 14 to 30 nucleotides. The ability to derive an antisense or a sense oligonucleotide, based upon a cDNA sequence encoding a given protein is described in, e.g., Stein & Cohen (Cancer Res. 48:2659 (1988 and van der Krol et al (BioTechniques 6:958 (1988)).

Ribozymes

In addition to antisense polynucleotides, ribozymes can be used to target and inhibit transcription of breast cancer-associated nucleotide sequences. A ribozyme is an RNA molecule that catalytically cleaves other RNA molecules. Different kinds of ribozymes have been described, including group I ribozymes, hammerhead ribozymes, hairpin ribozymes, RNase P, and axhead ribozymes (see, e.g., Castanotto et al., Adv. in Pharmacology 25: 289-317 (1994) for a general review of the properties of different ribozymes).

The general features of hairpin ribozymes are described, e.g., in Hampel et al., Nucl. Acids Res. 18:299-304 (1990); European Patent Publication No. 0 360 257; U.S. Pat. No. 5,254,678. Methods of preparing are well known to those of skill in the art (see, e.g., WO 94/26877; Ojwang et al., Proc. Natl. Acad. Sci. USA 90:6340-6344 (1993); Yamada et al., Human Gene Therapy 1:39-45 (1994); Leavitt et al., Proc. Natl. Acad. Sci. USA 92:699-703 (1995); Leavitt et al., Human Gene Therapy 5:1151-120 (1994); and Yamada et al., Virology 205: 121-126 (1994)).

Polynucleotide modulators of breast cancer may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a polynucleotide modulator of breast cancer may be introduced into a cell containing the target nucleic acid sequence, e.g., by formation of an polynucleotide-lipid complex, as described in WO 90/10448. It is understood that the use of antisense molecules or knock out and knock in models may also be used in screening assays as discussed above, in addition to methods of treatment.

Thus, in one embodiment, methods of modulating breast cancer in cells or organisms are provided. In one embodiment, the methods comprise administering to a cell an anti-breast cancer antibody that reduces or eliminates the biological activity of an endogenous breast cancer protein. Alternatively, the methods comprise administering to a cell or organism a recombinant nucleic acid encoding a breast cancer protein. This may be accomplished in any number of ways. In a preferred embodiment, e.g. when the breast cancer sequence is down-regulated in breast cancer, such state may be reversed by increasing the amount of breast cancer gene product in the cell. This can be accomplished, e.g., by overexpressing the endogenous breast cancer gene or administering a gene encoding the breast cancer sequence, using known gene-therapy techniques, e.g. In a preferred embodiment, the gene therapy techniques include the incorporation of the exogenous gene using enhanced homologous recombination (EHR), e.g. as described in PCT/US93/03868, hereby incorporated by reference in its entirety. Alternatively, e.g. when the breast cancer sequence is up-regulated in breast cancer, the activity of the endogenous breast cancer gene is decreased, e.g. by the administration of a breast cancer antisense nucleic acid.

In one embodiment, the breast cancer proteins of the present invention may be used to generate polyclonal and monoclonal antibodies to breast cancer proteins. Similarly, the breast cancer proteins can be coupled, using standard technology, to affinity chromatography columns. These columns may then be used to purify breast cancer antibodies useful for production, diagnostic, or therapeutic purposes. In a preferred embodiment, the antibodies are generated to epitopes unique to a breast cancer protein; that is, the antibodies show little or no cross-reactivity to other proteins. The breast cancer antibodies may be coupled to standard affinity chromatography columns and used to purify breast cancer proteins. The antibodies may also be used as blocking polypeptides, as outlined above, since they will specifically bind to the breast cancer protein.

Methods of Identifying Variant Breast Cancer-Associated Sequences

Without being bound by theory, expression of various breast cancer sequences is correlated with breast cancer. Accordingly, disorders based on mutant or variant breast cancer genes may be determined. In one embodiment, the invention provides methods for identifying cells containing variant breast cancer genes, e.g., determining all or part of the sequence of at least one endogenous breast cancer genes in a cell. This may be accomplished using any number of sequencing techniques. In a preferred embodiment, the invention provides methods of identifying the breast cancer genotype of an individual, e.g., determining all or part of the sequence of at least one breast cancer gene of the individual. This is generally done in at least one tissue of the individual, and may include the evaluation of a number of tissues or different samples of the same tissue. The method may include comparing the sequence of the sequenced breast cancer gene to a known breast cancer gene, i.e., a wild-type gene.

The sequence of all or part of the breast cancer gene can then be compared to the sequence of a known breast cancer gene to determine if any differences exist. This can be done using any number of known homology programs, such as Bestfit, etc. In a preferred embodiment, the presence of a difference in the sequence between the breast cancer gene of the patient and the known breast cancer gene correlates with a disease state or a propensity for a disease state, as outlined herein.

In a preferred embodiment, the breast cancer genes are used as probes to determine the number of copies of the breast cancer gene in the genome.

In another preferred embodiment, the breast cancer genes are used as probes to determine the chromosomal localization of the breast cancer genes. Information such as chromosomal localization finds use in providing a diagnosis or prognosis in particular when chromosomal abnormalities such as translocations, and the like are identified in the breast cancer gene locus.

Administration of Pharmaceutical and Vaccine Compositions

In one embodiment, a therapeutically effective dose of a breast cancer protein or modulator thereof, is administered to a patient. By “therapeutically effective dose” herein is meant a dose that produces effects for which it is administered. The exact dose will depend on the purpose of the treatment, and will be ascertainable by one skilled in the art using known techniques (e.g., Ansel et al., Pharmaceutical Dosage Forms and Drug Delivery; Lieberman, Pharmaceutical Dosage Forms (vols. 1-3, 1992), Dekker, ISBN 0824770846, 082476918X, 0824712692, 0824716981; Lloyd, The Art, Science and Technology of Pharmaceutical Compounding (1999); and Pickar, Dosage Calculations (1999)). As is known in the art, adjustments for breast cancer degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art. U.S. patent application Ser. No. 09/687,576, further discloses the use of compositions and methods of diagnosis and treatment in breast cancer is hereby expressly incorporated by reference.

A “patient” for the purposes of the present invention includes both humans and other animals, particularly mammals. Thus the methods are applicable to both human therapy and veterinary applications. In the preferred embodiment the patient is a mammal, preferably a primate, and in the most preferred embodiment the patient is human.

The administration of the breast cancer proteins and modulators thereof of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, e.g., in the treatment of wounds and inflammation, the breast cancer proteins and modulators may be directly applied as a solution or spray.

The pharmaceutical compositions of the present invention comprise a breast cancer protein in a form suitable for administration to a patient. In the preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that breast cancer protein modulators (e.g., antibodies, antisense constructs, ribozymes, small organic molecules, etc.) when administered orally, should be protected from digestion. This is typically accomplished either by complexing the molecule(s) with a composition to render it resistant to acidic and enzymatic hydrolysis, or by packaging the molecule(s) in an appropriately resistant carrier, such as a liposome or a protection barrier. Means of protecting agents from digestion are well known in the art.

The compositions for administration will commonly comprise a breast cancer protein modulator dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacologial Basis of Therapeutics (Hardman et al., eds., 1996)).

Thus, a typical pharmaceutical composition for intravenous administration would be about 0.1 to 10 mg per patient per day. Dosages from 0.1 up to about 100 mg per patient per day may be used, particularly when the drug is administered to a secluded site and not into the blood stream, such as into a body cavity or into a lumen of an organ. Substantially higher dosages are possible in topical administration. Actual methods for preparing parenterally administrable compositions will be known or apparent to those skilled in the art, e.g., Remington 's Pharmaceutical Science and Goodman and Gillman, The Pharmacologial Basis of Therapeutics, supra.

The compositions containing modulators of breast cancer proteins can be administered for therapeutic or prophylactic treatments. In therapeutic applications, compositions are administered to a patient suffering from a disease (e.g., a cancer) in an amount sufficient to cure or at least partially arrest the disease and its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the agents of this invention to effectively treat the patient. An amount of modulator that is capable of preventing or slowing the development of cancer in a mammal is referred to as a “prophylactically effective dose.” The particular dose required for a prophylactic treatment will depend upon the medical condition and history of the mammal, the particular cancer being prevented, as well as other factors such as age, weight, gender, administration route, efficiency, etc. Such prophylactic treatments may be used, e.g., in a mammal who has previously had cancer to prevent a recurrence of the cancer, or in a mammal who is suspected of having a significant likelihood of developing cancer.

It will be appreciated that the present breast cancer protein-modulating compounds can be administered alone or in combination with additional breast cancer modulating compounds or with other therapeutic agent, e.g., other anti-cancer agents or treatments.

In numerous embodiments, one or more nucleic acids, e.g., polynucleotides comprising nucleic acid sequences set forth in Tables 1-25, such as antisense polynucleotides or ribozymes, will be introduced into cells, in vitro or in vivo. The present invention provides methods, reagents, vectors, and cells useful for expression of breast cancer-associated polypeptides and nucleic acids using in vitro (cell-free), ex vivo or in vivo (cell or organism-based) recombinant expression systems.

The particular procedure used to introduce the nucleic acids into a host cell for expression of a protein or nucleic acid is application specific. Many procedures for introducing foreign nucleotide sequences into host cells may be used. These include the use of calcium phosphate transfection, spheroplasts, electroporation, liposomes, microinjection, plasma vectors, viral vectors and any of the other well known methods for introducing cloned genomic DNA, cDNA, synthetic DNA or other foreign genetic material into a host cell (see, e.g., Berger & Kimmel, Guide to Molecular Cloning Techniques, Methods in Enzymology volume 152 (Berger), Ausubel et al, eds., Current Protocols (supplemented through 1999), and Sambrook et al., Molecular Cloning—A Laboratory Manual (2nd ed., Vol. 1-3, 1989.

In a preferred embodiment, breast cancer proteins and modulators are administered as therapeutic agents, and can be formulated as outlined above. Similarly, breast cancer genes (including both the full-length sequence, partial sequences, or regulatory sequences of the breast cancer coding regions) can be administered in a gene therapy application. These breast cancer genes can include antisense applications, either as gene therapy (i.e. for incorporation into the genome) or as antisense compositions, as will be appreciated by those in the art.

Breast cancer polypeptides and polynucleotides can also be administered as vaccine compositions to stimulate HTL, CTL and antibody responses. Such vaccine compositions can include, e.g., lipidated peptides (see, e.g., Vitiello, A. et al., J. Clin. Invest. 95:341 (1995)), peptide compositions encapsulated in poly(DL-lactide-co-glycolide) (“PLG”) microspheres (see, e.g., Eldridge, et al., Molec. Immunol. 28:287-294, (1991); Alonso et al., Vaccine 12:299-306 (1994); Jones et al., Vaccine 13:675-681 (1995)), peptide compositions contained in immune stimulating complexes (ISCOMS) (see, e.g., Takahashi et al., Nature 344:873-875 (1990); Hu et al., Clin Exp Immunol. 113:235-243 (1998)), multiple antigen peptide systems (MAPs) (see, e.g., Tam, Proc. Natl. Acad. Sci. U.S.A. 85:5409-5413 (1988); Tam, J. Immunol. Methods 196:17-32 (1996)), peptides formulated as multivalent peptides; peptides for use in ballistic delivery systems, typically crystallized peptides, viral delivery vectors (Perkus, et al., In: Concepts in vaccine development (Kaufmann, ed., p. 379, 1996); Chakrabarti, et al., Nature 320:535 (1986); Hu et al., Nature 320:537 (1986); Kieny, et al., AIDS Bio/Technology 4:790 (1986); Top et al., J. Infect. Dis. 124:148 (1971); Chanda et al., Virology 175:535 (1990)), particles of viral or synthetic origin (see, e.g., Kofler et al., J. Immunol. Methods. 192:25 (1996); Eldridge et al., Sem. Hematol. 30:16 (1993); Falo et al., Nature Med. 7:649 (1995)), adjuvants (Warren et al., Annu. Rev. Immunol. 4:369 (1986); Gupta et al., Vaccine 11:293 (1993)), liposomes (Reddy et al., J. Immunol. 148:1585 (1992); Rock, Immunol. Today 17:131 (1996)), or, naked or particle absorbed cDNA (Ulmer, et al., Science 259:1745 (1993); Robinson et al., Vaccine 11:957 (1993); Shiver et al., In: Concepts in vaccine development (Kaufmann, ed., p. 423, 1996); Cease & Berzofsky, Annu. Rev. Immunol. 12:923 (1994) and Eldridge et al., Sem. Hematol. 30:16 (1993)). Toxin-targeted delivery technologies, also known as receptor mediated targeting, such as those of Avant Immunotherapeutics, Inc. (Needham, Mass.) may also be used.

Vaccine compositions often include adjuvants. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, e.g., Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

Vaccines can be administered as nucleic acid compositions wherein DNA or RNA encoding one or more of the polypeptides, or a fragment thereof, is administered to a patient. This approach is described, for instance, in Wolff et. al., Science 247:1465 (1990) as well as U.S. Pat. Nos. 5,580,859; 5,589,466; 5,804,566; 5,739,118; 5,736,524; 5,679,647; WO 98/04720; and in more detail below. Examples of DNA-based delivery technologies include “naked DNA”, facilitated (bupivicaine, polymers, peptide-mediated) delivery, cationic lipid complexes, and particle-mediated (“gene gun”) or pressure-mediated delivery (see, e.g., U.S. Pat. No. 5,922,687).

For therapeutic or prophylactic immunization purposes, the peptides of the invention can be expressed by viral or bacterial vectors. Examples of expression vectors include attenuated viral hosts, such as vaccinia or fowlpox. This approach involves the use of vaccinia virus, e.g., as a vector to express nucleotide sequences that encode breast cancer polypeptides or polypeptide fragments. Upon introduction into a host, the recombinant vaccinia virus expresses the immunogenic peptide, and thereby elicits an immune response. Vaccinia vectors and methods useful in immunization protocols are described in, e.g., U.S. Pat. No. 4,722,848. Another vector is BCG (Bacille Calmette Guerin). BCG vectors are described in Stover et al., Nature 351:456-460 (1991). A wide variety of other vectors useful for therapeutic administration or immunization e.g. adeno and adeno-associated virus vectors, retroviral vectors, Salmonella typhi vectors, detoxified anthrax toxin vectors, and the like, will be apparent to those skilled in the art from the description herein (see, e.g., Shata et al., Mol Med Today 6:66-71 (2000); Shedlock et al., J Leukoc Biol 68:793-806 (2000); Hipp et al., In Vivo 14:571-85 (2000)).

Methods for the use of genes as DNA vaccines are well known, and include placing a breast cancer gene or portion of a breast cancer gene under the control of a regulatable promoter or a tissue-specific promoter for expression in a breast cancer patient. The breast cancer gene used for DNA vaccines can encode full-length breast cancer proteins, but more preferably encodes portions of the breast cancer proteins including peptides derived from the breast cancer protein. In one embodiment, a patient is immunized with a DNA vaccine comprising a plurality of nucleotide sequences derived from a breast cancer gene. For example, breast cancer-associated genes or sequence encoding subfragments of a breast cancer protein are introduced into expression vectors and tested for their immunogenicity in the context of Class I MHC and an ability to generate cytotoxic T cell responses. This procedure provides for production of cytotoxic T cell responses against cells which present antigen, including intracellular epitopes.

In a preferred embodiment, the DNA vaccines include a gene encoding an adjuvant molecule with the DNA vaccine. Such adjuvant molecules include cytokines that increase the immunogenic response to the breast cancer polypeptide encoded by the DNA vaccine. Additional or alternative adjuvants are available.

In another preferred embodiment breast cancer genes find use in generating animal models of breast cancer. When the breast cancer gene identified is repressed or diminished in cancer tissue, gene therapy technology, e.g., wherein antisense RNA directed to the breast cancer gene will also diminish or repress expression of the gene. Animal models of breast cancer find use in screening for modulators of a breast cancer-associated sequence or modulators of breast cancer. Similarly, transgenic animal technology including gene knockout technology, e.g. as a result of homologous recombination with an appropriate gene targeting vector, will result in the absence or increased expression of the breast cancer protein. When desired, tissue-specific expression or knockout of the breast cancer protein may be necessary.

It is also possible that the breast cancer protein is overexpressed in breast cancer. As such, transgenic animals can be generated that overexpress the breast cancer protein. Depending on the desired expression level, promoters of various strengths can be employed to express the transgene. Also, the number of copies of the integrated transgene can be determined and compared for a determination of the expression level of the transgene. Animals generated by such methods find use as animal models of breast cancer and are additionally useful in screening for modulators to treat breast cancer.

Kits for Use in Diagnostic and/or Prognostic Applications

For use in diagnostic, research, and therapeutic applications suggested above, kits are also provided by the invention. In the diagnostic and research applications such kits may include any or all of the following: assay reagents, buffers, breast cancer-specific nucleic acids or antibodies, hybridization probes and/or primers, antisense polynucleotides, ribozymes, dominant negative breast cancer polypeptides or polynucleotides, small molecules inhibitors of breast cancer-associated sequences etc. A therapeutic product may include sterile saline or another pharmaceutically acceptable emulsion and suspension base.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The present invention also provides for kits for screening for modulators of breast cancer-associated sequences. Such kits can be prepared from readily available materials and reagents. For example, such kits can comprise one or more of the following materials: a breast cancer-associated polypeptide or polynucleotide, reaction tubes, and instructions for testing breast cancer-associated activity. Optionally, the kit contains biologically active breast cancer protein. A wide variety of kits and components can be prepared according to the present invention, depending upon the intended user of the kit and the particular needs of the user. Diagnosis would typically involve evaluation of a plurality of genes or products. The genes will be selected based on correlations with important parameters in disease which may be identified in historical or outcome data.

### EXAMPLES

### Example 1

### Tissue Preparation, Labeling Chips, and Fingerprints

Purifying Total RNA from Tissue Sample Using TRIzol Reagent

The sample weight is first estimated. The tissue samples are homogenized in 1 ml of TRIzol per 50 mg of tissue using a homogenizer (e.g., Polytron 3 100). The size of the generator/probe used depends upon the sample amount. A generator that is too large for the amount of tissue to be homogenized will cause a loss of sample and lower RNA yield. A larger generator (e.g., 20 mm) is suitable for tissue samples weighing more than 0.6 g. Fill tubes should not be overfilled. If the working volume is greater than 2 ml and no greater than 10 ml, a 15 ml polypropylene tube (Falcon 2059) is suitable for homogenization.

Tissues should be kept frozen until homogenized. The TRIzol is added directly to the frozen tissue before homogenization. Following homogenization, the insoluble material is removed from the homogenate by centrifugation at 7500×g for 15 min. in a Sorvall superspeed or 12,000×g for 10 min. in an Eppendorf centrifuge at 4° C. The cleared homogenate is then transferred to a new tube(s). Samples may be frozen and stored at −60 to −70° C. for at least one month or else continue with the purification.

The next process is phase separation. The homogenized samples are incubated for 5 minutes at room temperature. Then, 0.2 ml of chloroform per 1 ml of TRIzol reagent is added to the homogenization mixture. The tubes are securely capped and shaken vigorously by hand (do not vortex) for 15 seconds. The samples are then incubated at room temp. for 2-3 minutes and next centrifuged at 6500 rpm in a Sorvall superspeed for 30 min. at 4° C.

The next process is RNA Precipitation. The aqueous phase is transferred to a fresh tube. The organic phase can be saved if isolation of DNA or protein is desired. Then 0.5 ml of isopropyl alcohol is added per 1 ml of TRIzol reagent used in the original homogenization. Then, the tubes are securely capped and inverted to mix. The samples are then incubated at room temp. for 10 minutes an centrifuged at 6500 rpm in Sorvall for 20 min. at 4° C.

The RNA is then washed. The supernatant is poured off and the pellet washed with cold 75% ethanol. 1 ml of 75% ethanol is used per 1 ml of the TRIzol reagent used in the initial homogenization. The tubes are capped securely and inverted several times to loosen pellet without vortexing. They are next centrifuged at <8000 rpm (<7500×g) for 5 minutes at 4° C.

The RNA wash is decanted. The pellet is carefully transferred to an Eppendorf tube (sliding down the tube into the new tube by use of a pipet tip to help guide it in if necessary). Tube(s) sizes for precipitating the RNA depending on the working volumes. Larger tubes may take too long to dry. Dry pellet. The RNA is then resuspended in an appropriate volume (e.g., 2-5 ug/ul) of DEPC H₂O. The absorbance is then measured.

The poly A+ mRNA may next be purified from total RNA by other methods such as Qiagen's RNeasy kit. The poly A⁺ mRNA is purified from total RNA by adding the oligotex suspension which has been heated to 37° C. and mixing prior to adding to RNA. The Elution Buffer is incubated at 70° C. If there is precipitate in the buffer, warm up the 2× Binding Buffer at 65° C. The the total RNA is mixed with DEPC-treated water, 2× Binding Buffer, and Oligotex according to Table 2 on page 16 of the Oligotex Handbook and next incubated for 3 minutes at 65° C. and 10 minutes at room temperature.

The preparation is centrifuged for 2 minutes at 14,000 to 18,000 g, preferably, at a “soft setting,” The supernatant is removed without disturbing Oligotex pellet. A little bit of solution can be left behind to reduce the loss of Oligotex. The supernatant is saved until satisfactory binding and elution of poly A⁺ mRNA has been found.

Then, the preparation is gently resuspended in Wash Buffer OW2 and pipetted onto the spin column and centrifuged at full speed (soft setting if possible) for 1 minute.

Next, the spin column is transferred to a new collection tube and gently resuspended in Wash Buffer OW2 and centrifuged as described herein.

Then, the spin column is transferred to a new tube and eluted with 20 to 100 ul of preheated (70° C.) Elution Buffer. The Oligotex resin is gently resuspended by pipetting up and down. The centrifugation is repeated as above and the elution repeated with fresh elution buffer or first eluate to keep the elution volume low.

The absorbance is next read to determine the yield, using diluted Elution Buffer as the blank.

Before proceeding with cDNA synthesis, the mRNA is precipitated before proceeding with cDNA synthesis, as components leftover or in the Elution Buffer from the Oligotex purification procedure will inhibit downstream enzymatic reactions of the mRNA. 0.4 vol. of 7.5 M NH4OAc+2.5 vol. of cold 100% ethanol is added and the preparation precipitated at −20° C. 1 hour to overnight (or 20-30 min. at −70° C.), and centrifuged at 14,000-16,000×g for 30 minutes at 4° C. Next, the pellet is washed with 0.5 ml of 80% ethanol (−20° C.) and then centrifuged at 14,000-16,000×g for 5 minutes at room temperature. The 80% ethanol wash is then repeated. The last bit of ethanol from the pellet is then dried without use of a speed vacuum and the pellet is then resuspended in DEPC H₂O at 1 ug/ul concentration.

Alternatively the RNA may be Purified Using Other Methods (e.g. Qiagen's RNeasy Kit).

No more than 100 ug is added to the RNeasy column. The sample volume is adjusted to 100 ul with RNase-free water. 350 ul Buffer RLT and then 250 ul ethanol (100%) are added to the sample. The preparation is then mixed by pipetting and applied to an RNeasy mini spin column for centrifugation (15 sec at >10,000 rpm). If yield is low, reapply the flowthrough to the column and centrifuge again.

Then, transfer column to a new 2 ml collection tube and add 500 ul Buffer RPE and centrifuge for 15 sec at >10,000 rpm. The flowthrough is discarded. 500 ul Buffer RPE and is then added and the preparation is centriuged for 15 sec at >10,000 rpm. The flowthrough is discarded. and the column membrane dried by centrifuging for 2 min at maximum speed. The column is transferred to a new 1.5-ml collection tube. 30-50 ul of RNase-free water is applied directly onto column membrane. The column is then centrifuged for 1 min at >10,000 rpm and the elution step repeated.

The absorbance is then read to determine yield. If necessary, the material may be ethanol precipitated with ammonium acetate and 2.5× volume 100% ethanol.

First Strand cDNA Synthesis

The first strand can be make using using Gibco's “SuperScript Choice System for cDNA Synthesis” kit. The starting material is 5 ug of total RNA or 1 ug of polyA+ mRNA1. For total RNA, 2 ul of SuperScript RT is used; for polyA+ mRNA, 1 ul of SuperScript RT is used. The final volume of first strand synthesis mix is 20 ul. The RNA should be in a volume no greater than 10 ul. The RNA is incubated with 1 ul of 100 pmol T7-T24 oligo for 10 min at 70° C. followed by addition on ice of 7 ul of: 4 ul 5× 1^{st} Strand Buffer, 2 ul of 0.1M DTT, and 1 ul of 10 mM dNTP mix. The preparation is then incubated at 37° C. for 2 min before addition of the SuperScript RT followed by incubation at 37° C. for 1 hour.

Second Strand Synthesis

For the second strand synthesis, place 1st strand reactions on ice and add: 91 ul DEPC H₂O; 30 ul 5× 2nd Strand Buffer; 3 ul 10 mM dNTP mix; 1 ul 10 U/ul E. coli DNA Ligase; 4 ul 10 U/ul E. coli DNA Polymerase; and 1 ul 2 U/ul RNase H. Mix and incubate 2 hours at 16° C. Add 2 ul T4 DNA Polymerase. Incubate 5 min at 16° C. Add 10 ul of 0.5M EDTA.

Cleaning up cDNA

The cDNA is purified using Phenol:Chloroform:Isoamyl Alcohol (25:24:1) and Phase-Lock gel tubes. The PLG tubes are centrifuged for 30 sec at maximum speed. The cDNA mix is then transferred to PLG tube. An equal volume of phenol:chloroform:isamyl alcohol is then added, the preparation shaken vigorously (no vortexing), and centrifuged for 5 minutes at maximum speed. The top aqueous solution is transferred to a new tube and ethanol precipitated by adding 7.5×5M NH4OAc and 2.5× volume of 100% ethanol. Next, it is centrifuged immediately at room temperature for 20 min, maximum speed. The supernatant is removed, and the pellet washed with 2× with cold 80% ethanol. As much ethanol wash as possible should be removed before air drying the pellet; and resuspending it in 3 ul RNase-free water.

In Vitro Transcription (IVT) and Labeling with Biotin

In vitro Transcription (IVT) and labeling with biotin is performed as follows: Pipet 1.5 ul of cDNA into a thin-wall PCR tube. Make NTP labeling mix by combining 2 ul T7 10×ATP (75 mM) (Ambion); 2 ul T7 10×GTP (75 mM) (Ambion); 1.5 ul T7 10×CTP (75 mM) (Ambion); 1.5 ul T7 10×UTP (75 mM) (Ambion); 3.75 ul 10 mM Bio-11-UTP (Boehringer-Mannheim/Roche or Enzo); 3.75 ul 10 mM Bio-16-CTP (Enzo); 2 ul 10×T7 transcription buffer (Ambion); and 2 ul 10×T7 enzyme mix (Ambion). The final volume is 20 ul. Incubate 6 hours at 37° C. in a PCR machine. The RNA can be furthered cleaned. Clean-up follows the previous instructions for RNeasy columns or Qiagen's RNeasy protocol handbook. The cRNA often needs to be ethanol precipitated by resuspension in a volume compatible with the fragmentation step.

Fragmentation is performed as follows. 15 ug of labeled RNA is usually fragmented. Try to minimize the fragmentation reaction volume; a 10 ul volume is recommended but 20 ul is all right. Do not go higher than 20 ul because the magnesium in the fragmentation buffer contributes to precipitation in the hybridization buffer. Fragment RNA by incubation at 94 C for 35 minutes in 1× Fragmentation buffer (5× Fragmentation buffer is 200 mM Tris-acetate, pH 8.1; 500 mM KOAc; 150 mM MgOAc). The labeled RNA transcript can be analyzed before and after fragmentation. Samples can be heated to 65° C. for 15 minutes and electrophoresed on 1% agarose/TBE gels to get an approximate idea of the transcript size range

For hybridization, 200 ul (10 ug cRNA) of a hybridization mix is put on the chip. If multiple hybridizations are to be done (such as cycling through a 5 chip set), then it is recommended that an initial hybridization mix of 300 ul or more be made. The hybridization mix is: fragment labeled RNA (50 ng/ul final conc.); 50 pM 948-b control oligo; 1.5 pM BioB; 5 pM BioC; 25 pM BioD; 100 pM CRE; 0.1 mg/ml herring sperm DNA; 0.5 mg/ml acetylated BSA; and 300 ul with 1×MES hyb buffer.

The hybridization reaction is conducted with non-biotinylated IVT (purified by RNeasy columns) (see example 1 for steps from tissue to IVT): The following mixture is prepared:

| | |
|---|---|
| IVT antisense RNA; 4 μg: | μl |
| Random Hexamers (1 μg/μl): | 4 μl |
| H₂O: | μl |
| | 14 μl |

Incubate the above 14 μl mixture at 70° C. for 10 min.; then put on ice.

The Reverse transcription procedure uses the following mixture:

| | |
|---|---|
| 0.1 M DTT: | 3 μl |
| 50X dNTP mix: | 0.6 μl |
| H₂O: | 2.4 μl |
| Cy3 or Cy5 dUTP (1 mM): | 3 μl |
| SS RT II (BRL): | 1 μl |
| | 16 μl |

The above solution is added to the hybridization reaction and incubated for 30 min., 42° C. Then, 1 μl SSII is added and incubated for another hour before being placed on ice.

The 50×dNTP mix contains 25 mM of cold dATP, dCTP, and dGTP, 10 mM of dTTP and is made by adding 25 μl each of 100MM dATP, dCTP, and dGTP; 10 μl of 100 mM dTTP to 15 μl H₂O.]

RNA degradation is performed as follows. Add 86 pl H₂O, 1.5 μl 1M NaOH/2 mM EDTA and incubate at 65° C., 10 min. For U-Con 30, 500 μl TE/sample spin at 7000 g for 10 min, save flow through for purification. For Qiagen purification, suspend u-con recovered material in 500 μl buffer PB and proceed using Qiagen protocol. For DNAse digestion, add 1 ul of 1/100 dilution of DNAse/30 ul Rx and incubate at 37° C. for 15 min. Incubate at 5 min 95° C. to denature the DNAse.

Sample Preparation

For sample preparation, add Cot-i DNA, 10 III; 50×dNTPs, 1 μl; 20×SSC, 2.3 μl; Na pyro phosphate, 7.5 μl; 10 mg/ml Herring sperm DNA; 1 ul of 1/10 dilution to 21.8 final vol. Dry in speed vac. Resuspend in 15 μl H₂O. Add 0.38 μl 10% SDS. Heat 95° C., 2 min and slow cool at room temp. for 20 min. Put on slide and hybridize overnight at 64° C. Washing after the hybridization: 3×SSC/0.03% SDS: 2 min., 37.5 ml 20×SSC+0.75 ml 10% SDS in 250 ml H₂O; 1×SSC: 5 min., 12.5 ml 20×SSC in 250 ml H₂O; 0.2×SSC: 5 min., 2.5 ml 20×SSC in 250 ml H₂O. Dry slides and scan at appropiate PMT's and charnels.

TABLE 1
FIG. 1 from BRCA 001 US
Table 1 shows genes, (incorporated in their entirety here and throughout the application
where primekeys are provided), downregulated in tumor tissue compared to normal breast
tissue.

| Pkey | ExAccn | UnigeneID | UnigeneTitle | R1 |
|---|---|---|---|---|
| 100472 | D90084 | Hs.1023 | pyruvate dehydrogenase (lipoamide) alpha | 5 |
| 100499 | T51986 | Hs.283108 | hemoglobin, gamma G | 10 |
| 100545 | M55405 | | gb: Homo sapiens mucin (MUC-3) mRNA, part | 5 |
| 100549 | BE142019 | Hs.222056 | Homo sapiens cDNA FLJ11572 fis, clone HE | 10 |
| 100613 | X52078 | Hs.101047 | transcription factor 3 (E2A immunoglobul | 5 |
| 100635 | BE259039 | Hs.129953 | Ewing sarcoma breakpoint region 1 | 5 |
| 100645 | X16841 | Hs.167988 | neural cell adhesion molecule 1 | 5 |
| 100654 | A03758 | | NM_000477*: Homo sapiens albumin (ALB), m | 10 |
| 100702 | L27065 | | gb: Human neurofibromatosis 2 (NF2) mRNA, | 5 |
| 100915 | M60832 | Hs.249239 | collagen, type VIII, alpha 2 | 5 |
| 100971 | BE379727 | Hs.83213 | fatty acid binding protein 4, adipocyte | 10 |
| 101125 | AJ250562 | Hs.82749 | transmembrane 4 superfamily member 2 | 5 |
| 101166 | M90424 | Hs.2099 | lipocalin 1 (protein migrating faster th | 5 |
| 101184 | NM_001674 | Hs.460 | activating transcription factor 3 | 10 |
| 101336 | NM_006732 | Hs.75678 | FBJ murine osteosarcoma viral oncogene h | 10 |
| 101367 | X03350 | Hs.4 | alcohol dehydrogenase 1B (class I), beta | 10 |
| 101447 | M21305 | | gb: Human alpha satellite and satellite 3 | 10 |
| 101461 | N98569 | Hs.76422 | phospholipase A2, group IIA (platelets, | 10 |
| 101511 | M27826 | Hs.267319 | endogenous retroviral protease | 10 |
| 101634 | AV650262 | Hs.75765 | GRO2 oncogene | 5 |
| 101736 | M74447 | Hs.502 | transporter 2, ATP-binding cassette, sub | 10 |
| 102208 | U22961 | | gb: Human mRNA clone with similarity to L | 10 |
| 102297 | NM_001504 | Hs.198252 | G protein-coupled receptor 9 | 5 |
| 102450 | U48251 | Hs.75871 | protein kinase C binding protein 1 | 10 |
| 102515 | U89337 | Hs.169886 | tenascin XB | 10 |
| 102571 | U60115 | Hs.239069 | four and a half LIM domains 1 | 5 |
| 102800 | AA313538 | | gb: EST185419 Colon carcinoma (HCC) cell | 10 |
| 102857 | NM_006744 | Hs.76461 | retinol-binding protein 4, interstitial | 10 |
| 102990 | AA829286 | Hs.332053 | serum amyloid A1 | 10 |
| 103434 | X98085 | Hs.54433 | tenascin R (restrictin, janusin) | 5 |
| 103747 | AA081995 | | gb: zn26d06.r1 Stratagene neuroepithelium | 10 |
| 103750 | AA126129 | | gb: zm78c07.r1 Stratagene neuroepithelium | 5 |
| 103812 | AA137107 | Hs.326391 | Homo sapiens, clone MGC: 16638, mRNA, com | 10 |
| 103851 | AA326216 | Hs.8719 | hypothetical protein MGC1136 | 5 |
| 104080 | AB041036 | Hs.57771 | kallikrein 11 (KLK11; TLSP; PRSS20; hipp | 5 |
| 104093 | R50727 | Hs.336970 | ESTs | 10 |
| 104106 | AA422123 | | gb: zv26h12.r1 Soares_NhHMPu_S1 Homo sapi | 5 |
| 104109 | AL353957 | Hs.284181 | hypothetical protein DKFZp434P0531 | 10 |
| 104250 | F06638 | Hs.12440 | Homo sapiens clone 24734 mRNA sequence | 10 |
| 104340 | AA426189 | | gb: zw11e09.r1 Soares_NhHMPu_S1 Homo sapi | 5 |
| 104492 | N73185 | Hs.94285 | EST | 10 |
| 104506 | N91071 | Hs.109650 | ESTs | 10 |
| 104511 | N99542 | Hs.572 | orosomucoid 1 | 5 |
| 104532 | AI498763 | Hs.203013 | hypothetical protein FLJ12748 | 10 |
| 104536 | R24024 | Hs.158101 | Homo sapiens cDNA FLJ14673 fis, clone NT | 5 |
| 104572 | Y11312 | Hs.132463 | phosphoinositide-3-kinase, class 2, beta | 5 |
| 104659 | AW969769 | Hs.105201 | ESTs | 5 |
| 104677 | AA009764 | Hs.190380 | ESTs | 10 |
| 104711 | AA017245 | Hs.32794 | ESTs | 10 |
| 104731 | AA019300 | Hs.125070 | ESTs, Moderately similar to I54374 gene | 10 |
| 104764 | AI039243 | Hs.278585 | ESTs | 5 |
| 105005 | AI298208 | Hs.28805 | ESTs | 10 |
| 105036 | AA130390 | Hs.25549 | hypothetical protein FLJ20898 | 10 |
| 105105 | R61532 | Hs.87016 | hypothetical protein FLJ22938 | 5 |
| 105231 | AW970043 | Hs.238039 | hypothetical protein FLJ11090 | 5 |
| 105239 | AA221036 | | gb: zr03f12.r1 Stratagene NT2 neuronal pr | 10 |
| 105921 | AA421973 | Hs.169119 | ESTs, Weakly similar to T25731 hypotheti | 5 |
| 105957 | BE242857 | Hs.27021 | hypothetical protein FLJ11159 | 5 |
| 106052 | N79885 | Hs.6382 | ESTs, Highly similar to T00391 hypotheti | 10 |
| 106119 | AL359624 | Hs.11387 | KIAA1453 protein | 5 |
| 106181 | AI803651 | Hs.191608 | ESTs | 10 |
| 106194 | AW976171 | Hs.286194 | hypothetical protein FLJ22233 | 5 |
| 106283 | AI085846 | Hs.25522 | KIAA1808 protein | 10 |
| 106379 | AL042069 | Hs.119021 | DKFZP434N061 protein | 10 |
| 106451 | AW235928 | Hs.313182 | ESTs | 10 |
| 106491 | AA135688 | Hs.10083 | Homo sapiens, clone IMAGE: 4139786, mRNA, | 10 |
| 106700 | AA906434 | Hs.3776 | zinc finger protein 216 | 5 |
| 106782 | AW054886 | Hs.25682 | Homo sapiens mRNA for KIAA1863 protein, | 10 |
| 106851 | AI458623 | | gb: tk04g09.x1 NCI_CGAP_Lu24 Homo sapiens | 5 |
| 106870 | AI983730 | Hs.26530 | serum deprivation response (phosphatidyl | 5 |
| 106892 | AI347578 | Hs.124015 | hypothetical protein MGC2605 | 5 |
| 106954 | AF128847 | Hs.204038 | indolethylamine N-methyltransferase | 5 |
| 106991 | AJ223811 | Hs.30127 | hypothetical protein | 5 |
| 107103 | AI446183 | Hs.9572 | ESTs, Highly similar to CYA5_HUMAN ADENY | 5 |
| 107124 | AB006532 | Hs.31442 | RecQ protein-like 4 | 10 |
| 107148 | AI005036 | Hs.334305 | GS1999full | 10 |
| 107214 | AF127026 | Hs.5394 | myosin IA | 10 |
| 107242 | AB020672 | Hs.175411 | KIAA0865 protein | 10 |
| 107331 | AI905985 | Hs.111805 | ESTs | 10 |
| 107351 | U51704 | Hs.323428 | ESTs, Moderately similar to ALU8_HUMAN A | 5 |
| 107423 | W26652 | Hs.6163 | PTEN induced putative kinase 1 | 5 |
| 107447 | W28516 | Hs.19210 | hypothetical protein MGC11308 | 10 |
| 107451 | AL042425 | Hs.283976 | hypthetical protein PRO2389 | 10 |
| 107453 | AI092790 | Hs.334703 | hypothetical protein FLJ14529 | 5 |
| 107459 | W38002 | | Empirically selected from AFFX single pr | 10 |
| 107683 | N53167 | Hs.47623 | ESTs | 10 |
| 107711 | W96141 | Hs.220687 | ESTs | 10 |
| 107754 | AA017462 | Hs.269244 | ESTs | 10 |
| 107757 | BE621721 | Hs.280792 | hypothetical protein FLJ12387 similar to | 10 |
| 107864 | AA025060 | Hs.61246 | ESTs | 10 |
| 107872 | BE271708 | Hs.95110 | ESTs, Weakly similar to A55943 1-phospha | 10 |
| 107888 | AA025836 | Hs.191637 | ESTs | 5 |
| 107997 | AL049176 | Hs.82223 | chordin-like | 10 |
| 108056 | AA043675 | Hs.62633 | ESTs | 10 |
| 108081 | AA093668 | Hs.28578 | muscleblind (Drosophila)-like | 5 |
| 108113 | AA012881 | Hs.72531 | hypothetical protein FLJ11838 | 10 |
| 108238 | AA059473 | Hs.66783 | EST | 10 |
| 108257 | AA677927 | Hs.144269 | ESTs | 5 |
| 108335 | AA070500 | | gb: zm70h03.s1 Stratagene neuroepithelium | 5 |
| 108351 | AA071193 | | gb: zf79b12.s1 Soares_pineal_gland_N3HPG | 10 |
| 108382 | NM_006770 | Hs.67726 | macrophage receptor with collagenous str | 5 |
| 108392 | AA075124 | | gb: zm86a01.s1 Stratagene ovarian cancer | 10 |
| 108441 | AA079079 | | gb: zm97c09.s1 Stratagene colon HT29 (937 | 10 |
| 108446 | AA085383 | | gb: zn13g03.s1 Stratagene hNT neuron (937 | 10 |
| 108497 | AA074897 | | gb: zm85a05.r1 Stratagene ovarian cancer | 10 |
| 108604 | AA934589 | Hs.49696 | ESTs | 5 |
| 108662 | AF117646 | Hs.156637 | Cas-Br-M (murine) ectropic retroviral tr | 5 |
| 108706 | AA121820 | Hs.74569 | KIAA0842 protein | 10 |
| 108738 | AA126583 | Hs.158725 | ESTs | 10 |
| 108827 | AI273692 | Hs.110470 | ESTs | 10 |
| 109123 | AI028376 | Hs.73232 | ESTs | 10 |
| 109389 | AA101325 | Hs.86154 | hypothetical protein FLJ12457 | 10 |
| 109546 | F01449 | Hs.26954 | Homo sapiens mRNA; cDNA DKFZp762G123 (fr | 5 |
| 109919 | R40604 | Hs.129539 | ESTs, Weakly similar to MCAT_HUMAN MITOC | 10 |
| 110006 | AI094674 | Hs.30524 | ring finger protein 24 | 10 |
| 110141 | H46749 | Hs.31540 | ESTs | 10 |
| 110354 | W22165 | Hs.22586 | ESTs | 5 |
| 110433 | AW294162 | Hs.301062 | UDP-N-acetyl-alpha-D-galactosamine: polyp | 10 |
| 110448 | H51276 | Hs.13526 | hypothetical protein FLJ12688 | 10 |
| 110455 | H52576 | | gb: yt85e08.r1 Soares_pineal_gland_N3HPG | 5 |
| 110540 | H72639 | Hs.167608 | ESTs | 5 |
| 110553 | H60593 | Hs.124990 | ESTs | 10 |
| 110976 | AL044174 | Hs.159526 | patched (Drosophila) homolog | 10 |
| 110987 | AI753316 | Hs.26034 | ESTs | 5 |
| 111158 | N66616 | Hs.138629 | H. sapiens mRNA for subtelomeric repeats | 5 |
| 111168 | AI798376 | | gb: tr34b07.x1 NCI_CGAP_Ov23 Homo sapiens | 10 |
| 111187 | AJ224864 | Hs.9688 | leukocyte membrane antigen | 5 |
| 111307 | AA641636 | Hs.37477 | ESTs, Weakly similar to T46908 hypotheti | 5 |
| 111400 | R00144 | Hs.189771 | ESTs | 10 |
| 111498 | AI168511 | | gb: ow90h09.s1 Soares_fetal_liver_spleen_{—} | 10 |
| 111651 | R16733 | Hs.20499 | ESTs | 10 |
| 111738 | R26065 | | gb: yh39d03.s1 Soares placenta Nb2HP Homo | 5 |
| 111803 | AA593731 | Hs.325823 | ESTs, Moderately similar to ALU5_HUMAN A | 10 |
| 111995 | R42333 | Hs.302292 | ESTs | 10 |
| 112071 | AL117490 | Hs.47225 | Ras-associated protein Rap1 | 10 |
| 112204 | NM_006668 | Hs.25121 | cytochrome P450, subfamily 46 (cholester | 10 |
| 112258 | R51889 | Hs.24990 | ESTs | 5 |
| 112490 | R31094 | Hs.24378 | ESTs | 10 |
| 112588 | R77302 | | gb: yi75h08.s1 Soares placenta Nb2HP Homo | 10 |
| 112654 | BE618629 | Hs.268809 | ESTs | 5 |
| 112784 | T98628 | Hs.191290 | ESTs | 5 |
| 112817 | AI057205 | Hs.14584 | ESTs | 5 |
| 112885 | AA581428 | Hs.5021 | EST | 10 |
| 112913 | T16837 | Hs.4241 | ESTs | 5 |
| 113149 | T51588 | | gb: yb27e06.s1 Stratagene fetal spleen (9 | 10 |
| 113174 | T54659 | Hs.301755 | Homo sapiens cDNA FLJ11465 fis, clone HE | 5 |
| 113203 | AA743563 | Hs.10305 | ESTs | 5 |
| 113299 | AW207424 | Hs.332594 | ESTs | 10 |
| 113367 | N92359 | Hs.14518 | ESTs, Moderately similar to A48752 B-cel | 10 |
| 113457 | R16763 | Hs.268679 | ESTs | 5 |
| 113563 | AA913635 | Hs.326413 | Homo sapiens cDNA FLJ20812 fis, clone AD | 10 |
| 113574 | R06874 | Hs.268628 | ESTs, Moderately similar to ALU1_HUMAN A | 5 |
| 113776 | AI791905 | Hs.95549 | hypothetical protein | 10 |
| 113790 | AI244311 | Hs.26912 | ESTs | 10 |
| 113807 | W07586 | Hs.8045 | ESTs | 3 |
| 113958 | W86195 | | gb: zh54e05.s1 Soares_fetal_liver_spleen_{—} | 10 |
| 114211 | Z39319 | Hs.27347 | EST | 10 |
| 114254 | AB018263 | Hs.180338 | tumor necrosis factor receptor superfami | 5 |
| 114349 | AA745978 | Hs.28273 | ESTs | 5 |
| 114449 | AA020736 | | gb: ze63b11.s1 Soares retina N2b4HR Homo | 5 |
| 114484 | AA034378 | Hs.267319 | endogenous retroviral protease | 5 |
| 114576 | AA065096 | | gb: zm50a02.s1 Stratagene fibroblast (937 | 5 |
| 114624 | AA081507 | | gb: zn05b10.r1 Stratagene hNT neuron (937 | 5 |
| 114844 | AA234826 | Hs.87386 | EST | 5 |
| 114906 | AA234462 | Hs.87350 | ESTs | 5 |
| 115624 | AK000725 | Hs.50579 | hypothetical protein FLJ20718 | 3 |
| 115666 | AF173081 | Hs.178215 | Vertebrate LIN7 homolog 1, Tax interacti | 5 |
| 115712 | AB020649 | Hs.74569 | KIAA0842 protein | 5 |
| 115889 | AA398841 | Hs.39850 | hypothetical protein FLJ20517 | 10 |
| 115949 | AI478427 | Hs.43125 | esophageal cancer related gene 4 protein | 10 |
| 116107 | AL133916 | Hs.172572 | hypothetical protein FLJ20093 | 10 |
| 116180 | AA463902 | Hs.13522 | ESTs, Weakly similar to I38022 hypotheti | 5 |
| 116267 | AW968703 | Hs.30085 | hypothetical protein FLJ23186 | 5 |
| 116291 | AW410377 | Hs.41502 | hypothetical protein FLJ21276 | 5 |
| 116527 | AW194253 | Hs.68607 | ESTs | 10 |
| 116659 | BE314852 | Hs.168694 | Homo sapiens clone 23763 unknown mRNA, p | 5 |
| 116708 | F10528 | Hs.70001 | ESTs, Moderately similar to JC6169 nucle | 5 |
| 117058 | AW801806 | | gb: IL5-UM0070-110400-062-g07 UM0070 Homo | 5 |
| 117151 | AI803656 | Hs.42373 | ESTs | 5 |
| 117226 | N20468 | | gb: yx39b10.s1 Soares melanocyte 2NbHM Ho | 10 |
| 117323 | AI472863 | Hs.43387 | ESTs | 5 |
| 117571 | N34417 | Hs.44584 | ESTs | 3 |
| 117624 | N26627 | Hs.82364 | ESTs, Weakly similar to JC4124 pregnancy | 5 |
| 117673 | N40551 | Hs.184043 | Homo sapiens Ets-1 binding protein (E1B) | 10 |
| 117847 | N49285 | Hs.182391 | ESTs | 10 |
| 117877 | AW263476 | Hs.44268 | myelin gene expression factor 2 | 10 |
| 117919 | BE222341 | Hs.279472 | ESTs | 5 |
| 118049 | N53145 | | gb: yv55f09.s1 Soares fetal liver spleen | 3 |
| 118413 | AW955696 | Hs.90960 | ESTs | 10 |
| 118613 | AI078236 | Hs.49688 | ESTs | 5 |
| 118664 | N70907 | Hs.230619 | EST | 10 |
| 118858 | AL122040 | Hs.102981 | Homo sapiens mRNA; cDNA DKFZp434G1972 (f | 3 |
| 118902 | AA993527 | Hs.293907 | hypothetical protein FLJ23403 | 5 |
| 119039 | AI160570 | Hs.252097 | pregnancy specific beta-1-glycoprotein 6 | 3 |
| 119159 | AF142419 | Hs.15020 | homolog of mouse quaking QKI (KH domain | 5 |
| 119216 | AA514422 | Hs.221849 | ESTs | 5 |
| 119317 | AK002001 | Hs.51305 | v-maf musculoaponeurotic fibrosarcoma (a | 10 |
| 119366 | T77892 | | gb: yd20f04.s1 Soares fetal liver spleen | 5 |
| 119378 | T81824 | Hs.90949 | EST | 5 |
| 119528 | W38051 | | Empirically selected from AFFX single pr | 10 |
| 119792 | AL049798 | Hs.80552 | dermatopontin | 3 |
| 119800 | AF086332 | Hs.58314 | ESTs | 10 |
| 119817 | AF088061 | Hs.159690 | ESTs | 5 |
| 119835 | AF086429 | Hs.58429 | ESTs | 5 |
| 119923 | AW803308 | Hs.62954 | ferritin, heavy polypeptide 1 | 5 |
| 119961 | U34249 | Hs.337461 | Human putative zinc finger protein (ZNFB | 5 |
| 120379 | AL042725 | | gb: DKFZp434B1822_r1 434 (synonym: htes3) | 10 |
| 120931 | AW136934 | Hs.97162 | ESTs | 5 |
| 121037 | AA907743 | Hs.142373 | ESTs | 5 |
| 121282 | AA401695 | Hs.97334 | ESTs | 5 |
| 121382 | AA405763 | Hs.111939 | Homo sapiens cDNA FLJ20470 fis, clone KA | 5 |
| 121764 | AA421452 | Hs.164851 | ESTs, Weakly similar to KIAA0926 protein | 5 |
| 122034 | AK000229 | Hs.98017 | Homo sapiens cDNA FLJ20222 fis, clone CO | 10 |
| 122441 | AA447555 | Hs.99116 | EST | 10 |
| 122756 | AA458945 | Hs.95898 | ESTs | 10 |
| 122771 | AW135093 | Hs.97282 | ESTs, Highly similar to G100_HUMAN 110 K | 5 |
| 123601 | AA609122 | Hs.112645 | Homo sapiens mRNA; cDNA DKFZp434D2472 (f | 5 |
| 123623 | AI024595 | Hs.97508 | a disintegrin and metalloproteinase doma | 5 |
| 123941 | AA621529 | | gb: af47a02.s1 Soares_total_fetus_Nb2HF8_{—} | 10 |
| 124215 | H62570 | | gb: yr44a01.r1 Soares fetal liver spleen | 5 |
| 124276 | H83465 | | gb: ys91a11.s1 Soares retina N2b5HR Homo | 5 |
| 124680 | AK001527 | Hs.163953 | hypothetical protein FLJ10665 | 5 |
| 125099 | NM_014312 | Hs.112377 | cortic al thymocyte receptor (X. laevis | 10 |
| 125121 | T98199 | Hs.48403 | hypothetical protein FLJ10847 | 10 |
| 125188 | BE299567 | Hs.271749 | ESTs, Moderately similar to ALU8_HUMAN A | 5 |
| 125284 | NM_002666 | Hs.103253 | perilipin | 10 |
| 125906 | BE256206 | Hs 17775 | p75NTR-associated cell death executor; o | 5 |
| 128484 | AA485421 | Hs.270503 | ESTs, Weakly similar to ALU7_HUMAN ALU S | 10 |
| 128511 | NM_002250 | Hs.10082 | potassium intermediate/small conductance | 10 |
| 128538 | R44214 | Hs.101189 | ESTs | 5 |
| 128606 | C16161 | Hs.283040 | hypothetical protein PRO2543 | 5 |
| 128850 | AA193106 | Hs.180817 | chromosome 11 open reading frame 23 | 10 |
| 128870 | H39537 | Hs.75309 | eukaryotic translation elongation factor | 10 |
| 128903 | AW150717 | Hs.296176 | STAT induced STAT inhibitor 3 | 10 |
| 128931 | N62889 | Hs.107242 | Homo sapiens cDNA FLJ12965 fis, clone NT | 10 |
| 129001 | AA443323 | Hs.107812 | BPOZ protein | 5 |
| 129091 | AA056483 | Hs.301463 | Human Chromosome 16 BAC clone CIT987SK-A | 5 |
| 129101 | NM_013403 | Hs.108665 | zinedin | 10 |
| 129146 | AL117472 | Hs.108924 | SH3-domain protein 5 (ponsin) | 5 |
| 129213 | AI146494 | Hs.109525 | ESTs, Weakly similar to IRX2_HUMAN IROQU | 3 |
| 129228 | U40714 | Hs.239307 | tyrosyl-tRNA synthetase | 5 |
| 129265 | AA530892 | Hs.171695 | dual specificity phosphatase 1 | 5 |
| 129285 | BE617015 | Hs.11006 | ESTs, Moderately similar to T17372 plasm | 10 |
| 129346 | AF110141 | Hs.288908 | WAS protein family, member 2 | 10 |
| 129368 | NM_003877 | Hs.110776 | STAT induced STAT inhibitor-2 | 5 |
| 129371 | X06828 | Hs.110802 | von Willebrand factor | 5 |
| 129381 | AW245805 | Hs.110903 | claudin 5 (transmembrane protein deleted | 10 |
| 129440 | W37944 | Hs.4007 | Sarcolemmal-associated protein | 5 |
| 129441 | BE061069 | Hs.301943 | KIAA0467 protein | 10 |
| 129516 | AF020038 | Hs.11223 | isocitrate dehydrogenase 1 (NADP+), solu | 10 |
| 129554 | BE222078 | Hs.113069 | ESTs | 10 |
| 129684 | BE622468 | Hs.11924 | ESTs, Weakly similar to I38022 hypotheti | 5 |
| 129702 | AI304966 | Hs.12035 | ESTs, Weakly similar to I38022 hypotheti | 5 |
| 129778 | AK001676 | Hs.12457 | hypothetical protein FLJ10814 | 10 |
| 129893 | AK000956 | Hs.13209 | hypothetical protein FLJ10094 | 5 |
| 129928 | AI338993 | Hs.134535 | ESTs | 5 |
| 129973 | AJ251760 | Hs.273385 | guanine nucleotide binding protein (G pr | 5 |
| 129977 | NM_000399 | Hs.1395 | early growth response 2 (Krox-20 (Drosop | 5 |
| 130014 | NM_001158 | Hs.143102 | amine oxidase, copper containing 2 (reti | 5 |
| 130085 | M62402 | Hs.274313 | insulin-like growth factor binding prote | 10 |
| 130089 | AA452006 | Hs.333199 | ESTs | 5 |
| 130162 | W80711 | Hs.319946 | Homo sapiens mRNA for KIAA1727 protein, | 5 |
| 130243 | D88435 | Hs.153227 | cyclin G associated kinase | 10 |
| 130315 | AI241084 | Hs.154353 | nonselective sodium potassium/proton exc | 5 |
| 130339 | AA435746 | | gb: zt79e03.s1 Soares_testis_NHT Homo sap | 5 |
| 130400 | V00517 | Hs.283108 | hemoglobin, gamma G | 10 |
| 130436 | NM_001928 | Hs.155597 | D component of complement (adipsin) | 10 |
| 130478 | X72308 | Hs.251526 | small inducible cytokine A7 (monocyte ch | 5 |
| 130480 | BE222978 | Hs.15760 | MYG1 protein | 10 |
| 130494 | AW390834 | Hs.75874 | pregnancy-associated plasma protein A | 5 |
| 130563 | BE270472 | Hs.279900 | HSPC015 protein | 10 |
| 130589 | AL110226 | Hs.16441 | DKFZP434H204 protein | 10 |
| 130606 | AI652143 | Hs.288382 | hypothetical protein FLJ13111 | 5 |
| 130634 | AI769067 | Hs.127824 | ESTs, Weakly similar to T28770 hypotheti | 3 |
| 130683 | AA993269 | Hs.17872 | Homo sapiens, clone IMAGE: 3875012, mRNA | 10 |
| 130689 | NM_006691 | Hs.17917 | extracellular link domain-containing 1 | 10 |
| 130716 | AA232075 | Hs.18259 | XPA binding protein 1; putative ATP(GTP) | 5 |
| 130718 | AF263462 | Hs.18376 | KIAA1319 protein | 10 |
| 130722 | N41322 | Hs.18441 | ESTs | 5 |
| 130798 | M81349 | Hs.1955 | serum amyloid A4, constitutive | 10 |
| 130840 | BE048821 | Hs.20144 | small inducible cytokine subfamily A (Cy | 10 |
| 131184 | AB040935 | Hs.23954 | cerebral cell adhesion molecule | 10 |
| 131261 | AA360419 | Hs.171776 | inositol(myo)-1(or 4)-monophosphatase 1 | 10 |
| 131282 | X03350 | Hs.4 | alcohol dehydrogenase 1B (class I), beta | 10 |
| 131328 | AW939251 | Hs.25647 | v-fos FBJ murine osteosarcoma viral onco | 10 |
| 131340 | AK000393 | Hs.25817 | BTB (POZ) domain containing 2 | 5 |
| 131341 | AF110908 | Hs.297660 | TNF receptor-associated factor 3 | 5 |
| 131406 | H83294 | Hs.284122 | Wnt inhibitory factor-1 | 5 |
| 131489 | BE394648 | Hs.27414 | hypothetical protein | 5 |
| 131543 | AW966881 | Hs.41639 | programmed cell death 2 | 10 |
| 131692 | BE559681 | Hs.30736 | KIAA0124 protein | 5 |
| 131753 | AA829286 | Hs.332053 | serum amyloid A1 | 10 |
| 131756 | AA443966 | Hs.31595 | ESTs | 10 |
| 131785 | H69342 | Hs.26320 | TRABID protein | 10 |
| 131815 | AA021258 | Hs.32753 | ESTs | 5 |
| 131819 | BE244961 | Hs.173103 | FE65-LIKE 2 | 5 |
| 131828 | AJ000263 | Hs.278658 | keratin, hair, basic, 6 (monilethrix) | 10 |
| 131888 | AW294659 | Hs.34054 | Homo sapiens cDNA: FLJ22488 fis, clone H | 5 |
| 131927 | AJ003112 | Hs.34780 | doublecortex; lissencephaly, X-linked (d | 5 |
| 131949 | AK000010 | Hs.258798 | hypothetical protein FLJ20003 | 10 |
| 132115 | H81604 | Hs.178471 | KIAA0798 gene product | 5 |
| 132177 | X80818 | Hs.178078 | glutamate receptor, metabotropic 4 | 5 |
| 132296 | AA467752 | Hs.195161 | ESTs | 5 |
| 132426 | AW118072 | Hs.89981 | diacylglycerol kinase, zeta (104 kD) | 10 |
| 132477 | S68874 | Hs.170917 | prostaglandin E receptor 3 (subtype EP3) | 5 |
| 132675 | AI291496 | Hs.5476 | Homo sapiens, clone IMAGE: 3530123, mRNA, | 10 |
| 132796 | NM_006283 | Hs.173159 | transforming, acidic coiled-coil contain | 10 |
| 132898 | W28548 | Hs.224829 | ESTs | 10 |
| 132905 | NM_004235 | Hs.7934 | Kruppel-like factor 4 (gut) | 10 |
| 132953 | BE175645 | Hs.321264 | LBP protein 32 | 5 |
| 133116 | BE563966 | Hs.6529 | ESTs, Weakly similar to I78885 serine/th | 5 |
| 133120 | NM_003278 | Hs.65424 | tetranectin (plasminogen-binding protein | 10 |
| 133139 | AF052138 | Hs.6580 | Homo sapiens cDNA: FLJ23227 fis, clone C | 5 |
| 133163 | AA668224 | Hs.6634 | Homo sapiens cDNA: FLJ22547 fis, clone H | 5 |
| 133268 | AW956781 | Hs.293937 | ESTs, Weakly similar to FXD2_HUMAN FORKH | 5 |
| 133272 | NM_002776 | Hs.69423 | kallikrein 10 (KLK10) (PRSSL1) (nes1) | 5 |
| 133379 | AA207059 | | gb: zq80h09.s1 Stratagene hNT neuron (937 | 5 |
| 133407 | AF017987 | Hs.7306 | secreted frizzled-related protein 1 | 5 |
| 133552 | H21497 | Hs.7471 | BBP-like protein 1 | 5 |
| 133702 | L02321 | Hs.75652 | glutathione S-transferase M5 | 5 |
| 133719 | H26904 | Hs.75736 | apolipoprotein D | 5 |
| 133731 | N71725 | Hs.272572 | hemoglobin, alpha 2 | 10 |
| 133789 | T85626 | Hs.76239 | hypothetical protein FLJ20608 | 5 |
| 134007 | AF072441 | Hs.7840 | calcineurin binding protein 1 | 10 |
| 134055 | D86062 | Hs.182423 | ES1 (zebrafish) protein, human homolog o | 10 |
| 134111 | AI372588 | Hs.8022 | TU3A protein | 10 |
| 134117 | AA081846 | Hs.7921 | Homo sapiens mRNA; cDNA DKFZp566E183 (fr | 10 |
| 134177 | BE243319 | Hs.79672 | KIAA0652 gene product | 5 |
| 134308 | AW905827 | Hs.81454 | ketohexokinase (fructokinase) | 10 |
| 134361 | BE549343 | Hs.82208 | acyl-Coenzyme A dehydrogenase, very long | 5 |
| 134369 | AF207664 | Hs.8230 | a disintegrin-like and metalloprotease ( | 5 |
| 134449 | L34155 | Hs.83450 | laminin, alpha 3 (nicein (150 kD), kalini | 5 |
| 134467 | AI190413 | Hs.8373 | ESTs | 10 |
| 134496 | M64936 | | gb: Homo sapiens retinoic acid-inducible | 10 |
| 134510 | NM_002757 | Hs.250870 | mitogen-activated protein kinase kinase | 10 |
| 134550 | M26315 | Hs.85258 | CD8 antigen, alpha polypeptide (p32) | 10 |
| 134577 | BE244323 | Hs.85951 | exportin, tRNA (nuclear export receptor | 5 |
| 134591 | U73394 | Hs.166085 | killer cell immunoglobulin-like receptor | 5 |
| 134678 | AL008583 | Hs.182595 | dynein, axonemal, light polypeptide 4 | 5 |
| 134728 | D10216 | Hs.89394 | POU domain, class 1, transcription facto | 5 |
| 134758 | NM_000078 | Hs.89538 | cholesteryl ester transfer protein, plas | 10 |
| 134786 | T29618 | Hs.89640 | TEK tyrosine kinase, endothelial (venous | 10 |
| 134912 | T87521 | Hs.261457 | ESTs | 5 |
| 134963 | NM_003394 | Hs.91985 | wingless-type MMTV integration site fami | 10 |
| 134969 | H22570 | Hs.172572 | hypothetical protein FLJ20093 | 5 |
| 135001 | AA302517 | Hs.92732 | KIAA1444 protein | 5 |
| 135066 | X04430 | Hs.93913 | interleukin 6 (interferon, beta 2) | 10 |
| 135173 | AL036557 | Hs.95910 | putative lymphocyte G0/G1 switch gene | 10 |
| 135197 | U76456 | Hs.190787 | tissue inhibitor of metalloproteinase 4 | 5 |
| 135219 | AB002361 | Hs.96633 | KIAA0363 protein | 5 |
| 135250 | U83171 | Hs.97203 | small inducible cytokine subfamily A (Cy | 5 |
| 135304 | AA416829 | Hs.191597 | ESTs | 5 |
| 135337 | AA905406 | Hs.9905 | ESTs, Weakly similar to unnamed protein | 3 |
| 135417 | X55019 | Hs.99975 | cholinergic receptor, nicotinic, delta p | 10 |
| 101367 | X03350 | Hs.4 | alcohol dehydrogenase 1B (class I), beta | 5 |
| 128870 | H39537 | Hs.75309 | eukaryotic translation elongation factor | 5 |
| 129381 | AW245805 | Hs.110903 | claudin 5 (transmembrane protein deleted | 5 |
| 130085 | M62402 | Hs.274313 | insulin-like growth factor binding prote | 5 |
| 130689 | NM_006691 | Hs.17917 | extracellular link domain-containing 1 | 10 |
| 133120 | NM_003278 | Hs.65424 | tetranectin (plasminogen-binding protein | 3 |
| 133407 | AF017987 | Hs.7306 | secreted frizzled-related protein 1 | 5 |
| 133731 | N71725 | Hs.272572 | hemoglobin, alpha 2 | 5 |
| 134369 | AF207664 | Hs.8230 | a disintegrin-like and metalloprotease ( | 5 |
| 135066 | X04430 | Hs.93913 | interleukin 6 (interferon, beta 2) | 10 |
| 135173 | AL036557 | Hs.95910 | putative lymphocyte G0/G1 switch gene | 5 |
| 322580 | AK001852 | Hs.274151 | ligatin | 5 |
| 408790 | AW580227 | Hs.47860 | neurotrophic tyrosine kinase, receptor, type 2 | 10 |
| 418043 | AW377752 | Hs.83341 | AXL receptor tyrosine kinase | 5 |
| 427458 | BE208364 | Hs.29283 | ESTs, Weakly similar to LKHU proteoglycan link | 5 |
| 446674 | AA563892 | Hs.306000 | solute carrier family 4 (anion exchanger), memb | 10 |
| 449826 | U85642 | Hs.138506 | ESTs | 5 |
| | RC_H15814_s | | Human apM1 mRNA for GS3109 (novel adipose specific collagen | 10 |
| | YEL024w/RIP1 | | EST - YEL024w/RIP1 | 3 |

Pkey: Unique Eos probeset identifier number
ExAccn: Exemplar Accession number, Genbank accession number
UnigeneID: Unigene number
Unigene Title: Unigene gene title
R1: Ratio of normal breast tissue to tumor

TABLE 1A
Table 1A shows the accession numbers for those pkeys lacking unigeneID's for Table 1.
For each probeset, we have listed the gene cluster number from which the oligonucleotides
were designed. Gene clusters were compiled using sequences derived from Genbank ESTs
and mRNAs. These sequences were clustered based on sequence similarity using Clustering
and Alignment Tools (DoubleTwist, Oakland California). The Genbank accession numbers
for sequences comprising each cluster are listed in the “Accession” column.

| | CAT | |
|---|---|---|
| Pkey | Number | Accessions |
| 108446 | 112224_1 | AA085383 AA126091 AA074174 AA075373 AA079120 AA070831 AA075978 AA075372 AA128503 |
| 108497 | 110079_2 | AA074897 AA113914 AA064871 AA079329 AA071309 AA084710 AA129030 AA075042 |
| | | AA074794 AA071453 AA078803 |
| | | AA148628 AA122204 AA074159 AA126185 AA079117 AA127089 AA070912 AA079280 |
| | | AA131372 AA078833 AA071087 |
| | | AA076131 AA071047 AA079401 AA083070 AA102076 AA115163 |
| | | AA074198 AA134725 AA113889 |
| | | AA121103 AA075041 |
| | | AA065148 AA071310 AA101144 AA079659 AA078931 AA079209 |
| | | AA070928 AA068994 AA069817 AA076187 AA069053 |
| | | AA131489 AA071308 AA063317 AA070156 AA071430 AA076056 |
| | | AA075684 AA070053 AA126283 AA126078 AA075895 |
| | | AA079208 AA074583 AA071086 AA079623 AA070627 AA078802 AA076622 |
| | | AA065051 AA079143 AA071110 AA079434 |
| | | AA148748 AA079230 AA085188 AA074485 AA070580 AA076151 AA083166 |
| | | AA085118 AA079450 AA085044 AA120938 |
| | | AA079200 AA100188 AA081472 AA122355 AA129031 AA085362 AA069220 |
| | | AA070940 AA075968 AA074563 AA084027 |
| | | AA115929 |
| 124215 | 1597154_1 | H62570 H59063 |
| 117058 | 1219924_1 | AW801806 H90434 BE086530 |
| 110455 | 46874_1 | H52576 AF085971 H52172 |
| 111168 | 38585_1 | AI798376 S46400 AW811617 AW811616 W00557 BE142245 |
| | | AW858232 AW861851 AW858362 AA232351 AA218567 |
| | | AA055556 AW858231 AW857541 AW814172 H66214 AW814398 AF134164 |
| | | AA243093 AA173345 AA199942 AA223384 |
| | | AA227092 AA227080 T12379 AA092174 T61139 AA149776 AA699829 AW879188 AW813567 |
| | | AW813538 AI267168 |
| | | AA157718 AA157719 AA100472 AA100774 AA130756 AA157705 AA157730 AA157715 |
| | | AA053524 AW849581 AW854566 |
| | | C05254 AW882836 T92637 AW812621 AA206583 AA209204 BE156909 AA226824 |
| | | AI829309 AW991957 N66951 AA527374 |
| | | H66215 AA045564 AI694265 H60808 AA149726 AW195620 BE081333 BE073424 |
| | | AW817662 AW817705 AW817703 |
| | | AW817659 BE081531 H59570 |
| 111498 | 411008_1 | AI168511 AI022712 AA700366 R07371 R07324 |
| 104340 | 46289_10 | AA426189 F15201 |
| 103747 | 117944_1 | AA081995 AA101099 |
| 134496 | 46501_1 | M64936 AI025512 AI382987 BE061777 AA089966 |
| | | BE169930 T41176 AW594624 BE502415 AA121893 AI269283 T40311 |
| | | AI684569 AA257011 |
| | | AI079277 AI241318 BE327710 AW975215 AW896268 AA884990 BE327514 |
| 103750 | 118365_1 | AA126129 AA126033 AA082561 |
| 105239 | 34624_1 | AA221036 R87170 BE537068 BE544757 |
| | | C18935 AW812058 T92565 AA227415 AA233942 AA223237 AA668403 AA601627 |
| | | AW869639 BE061833 BE000620 AW961170 AW847519 |
| | | AA308542 AW821833 AW945688 C04699 AA205504 AA377241 |
| | | AW821667 AA055720 AW817981 AW856468 AA155719 AA179928 |
| | | T03007 AW754298 AA227407 AA113928 AA307904 |
| | | C16859 |
| 120379 | 34624_3 | AL042725 BE063316 AW975610 AA457591 |
| | | BE062092 AI655202 AA714296 AI267264 AI075321 AA223286 AA071122 |
| | | AA227849 AA216700 AI696002 AA101867 AA099426 AA135997 |
| | | AL041698 T02815 T51824 AA207189 T59230 T51868 |
| | | AA663341 BE165757 AW818104 AW392886 AA584918 AA099408 AW856396 AW861859 AA053045 |
| 114624 | 111686_1 | AA081507 AA070071 AA070840 AA084362 |
| 106851 | 322947_1 | AI458623 AA639708 AA485409 R22065 AA485570 |
| 108392 | 113549_1 | AA075124 AA075208 |
| 100545 | 22955_11 | M55405 AW752552 |
| 100654 | tigr_{—} | A03758 A06977 A15293 D17029 D17107 D17171 L00132 |
| | | L00133 M12523 M13075 M13076 M92816 U22961 V00494 V00495 |
| | HT2969 | X51363 X51364 X51365 |
| 100702 | tigr_{—} | L27065 |
| | HT3413 | |
| 102208 | 6735_9 | U22961 AA203623 AA503337 AI174733 |
| | | AI192802 C06092 AA035357 AI190619 AI199244 AI828450 AA602296 AI378195 |
| | | AI209170 AI186653 AI127795 AI183846 H77389 AI589465 AA629390 |
| | | H94306 AI018388 R68584 AA027196 AI745413 |
| | | AI685092 AI093426 AI623873 AI074570 N50096 AA047486 N25060 |
| | | AA327614 AI042512 AI383957 AA156873 AI333101 |
| | | N70806 AI141254 AI383191 AI401237 AI080709 AI093400 W84549 |
| | | T90806 R00012 W01413 AA630557 AI378348 AI559265 |
| | | AA877103 W84464 AA625146 R68379 AI133207 AI132980 AI133214 |
| | | AI064826 AI061615 AI133473 AI174852 AI133404 |
| | | AI133272 V00494 M12523 M12523 AI207526 AI133120 AI064802 |
| | | AI174993 AI114729 AI061645 AI064716 AI064959 H77388 |
| | | T85706 AF075298 AI110799 D17107 NM_000477 AF190168 |
| | | R50724 AI248416 AI207432 AI133684 AI133345 AI174710 |
| | | AI133290 AI133304 AI174948 AI207484 AI110717 AF074624 |
| | | AI114515 AF063516 AI110642 AI114559 AI114498 AI114759 |
| | | AI207568 AI064960 AI174753 AI114666 R69184 R00011 AI064997 |
| | | T60501 AI207701 T71735 AA385318 H73569 T60496 |
| | | H94399 AI133158 T74675 AA484750 T73413 T55909 R50261 T72061 |
| | | N80533 T51189 T74936 AI207490 AI132925 AI064701 |
| | | AI174748 AI114663 AI133104 AI132999 AI133100 AI064925 |
| | | AI064979 AI133063 AA343347 T69091 AA233989 T39772 |
| | | AI444620 T52290 D16931 T40012 T48403 T58926 T69195 |
| | | AI133061 T50850 AI400677 AI091136 AA334608 T57411 Z20979 |
| | | N56507 T87485 AI133622 AA343370 T40075 T69671 T53849 |
| | | T74820 AF075316 AI110818 T40121 T57381 AI114468 |
| | | AA332728 T51362 AI114589 R06691 AI110629 AF063503 |
| | | AI140543 AA334661 AA332720 AA343262 T73513 T86549 |
| | | AI114840 T57284 T39981 T61407 T72757 T51749 T56630 |
| | | AA343125 T72126 R94135 T83028 T39972 T39896 AI174786 |
| | | AI132926 R09237 AI064838 AI133660 T60398 T88753 T55930 |
| | | T92126 AI444602 T60996 AI114792 H93911 AI133106 |
| | | R10779 AI065020 T90925 T50889 D17029 AI133703 AA333805 |
| | | AI133040 AI133017 AI064857 AI110730 AF074637 AI207567 |
| | | H71080 T73217 AA343950 AI174743 AA334224 AA334281 |
| | | R06692 T64739 T40163 T60628 T81661 T73179 R01842 |
| | | AA501730 T39931 T39662 T40136 AA334904 T71425 H77784 |
| | | R00874 AI065049 T84512 T55918 AI207595 T39951 |
| | | AA005016 T60361 T69176 T73356 T58795 T61233 T39955 |
| | | T60612 AI114676 AI064778 AA035710 W52763 AI114786 |
| | | T83564 AA341859 T81684 T55769 AI114710 T51776 AA343213 |
| | | AI114714 T58102 AI110809 R28984 AI174854 AA305675 |
| | | AA343592 T53836 T46869 T64721 T55508 W05241 T54019 |
| | | T57945 T60513 T48364 AF075308 W86731 T82851 T48269 |
| | | H54053 T73211 AI114590 T48317 T55965 T74857 R84226 |
| | | T56552 T52231 T74946 T76976 R02576 T95666 AI203974 |
| | | AI189471 AA005147 AI478102 AI207662 AI192792 AI768421 |
| | | AI064737 AW051713 AA936693 AI133117 AI766232 AI913646 |
| | | T83962 AI065112 AI207689 AI174684 AI207702 T81475 AI133325 |
| | | AI032512 AA701169 AI936354 AI114720 AI433289 |
| | | AA046980 AI823482 AI114536 AA860651 AW242644 R07469 AW300438 |
| | | AI133416 AW271670 AI991363 T78943 AI823481 |
| | | AA845518 AA719124 AA883454 T68850 T69115 AI935509 AI150977 |
| | | T62890 T71374 T68294 AI174774 T67411 T68318 |
| | | AI064689 T56624 T69010 T68982 T68302 AI332829 T72908 AI064819 |
| | | AI205880 T62895 T69430 T95111 AA025050 T73330 |
| | | W52657 T71984 T69118 W92684 AI114860 T62093 T61797 |
| | | AI522333 T73322 H92981 T56018 T61811 T57232 AI336158 |
| | | T61821 T69457 T62900 T62912 T72917 T46885 AI702448 |
| | | T57212 T57203 R94581 T71311 T61819 T89358 T67708 T70918 |
| | | T59166 AI187111 T64308 T62071 T69427 AI114750 T60430 |
| | | R09734 T69033 T69141 T69453 T67908 R16809 T69394 |
| | | AI207729 T55839 T90273 T73339 AW194909 T75486 T71850 |
| | | T71305 T71287 T53877 T73452 T68852 N75290 AI312890 |
| | | T67751 AI174983 T51679 T54851 H69880 N73734 AA443453 T73466 |
| | | H69672 N53869 T68447 D11809 D12412 T64300 |
| | | T28321 T55864 |
| 123941 | genbank_AA621529 | AA621529 |
| 118049 | genbank_N53145 | N53145 |
| 102800 | 14782_20 | AA313538 U88895 U88902 |
| 104106 | AA422123_i_{—} | |
| | atAA422123_i | |
| 111738 | genbank_R26065 | R26065 |
| 113149 | genbank_T51588 | T51588 |
| 113958 | genbank_W86195 | W86195 |
| 108335 | genbank_AA070500 | AA070500 |
| 108351 | genbank_AA071193 | AA071193 |
| 108441 | genbank_AA079079 | AA079079 |
| 124276 | genbank_H83465 | H83465 |
| 101447 | entrez_M21305 | M21305 |
| 117226 | genbank_N20468 | N20468 |
| 133379 | genbank_AA207059 | AA207059, AA207241 |
| 119366 | genbank_T77892 | T77892 |
| 119528 | NOT_FOUND_{—} | W38051 |
| | entrez_W38051 | |
| 112588 | genbank_R77302 | R77302 |
| 114449 | genbank_AA020736 | AA020736 |
| 114576 | genbank_AA065096 | AA065096 |
| 107459 | W38002_s_at | W38002_s |
| 130339 | genbank_AA435746 | AA435746 |

Pkey: Unique Eos probeset identifier number
CAT number: Gene cluster number
Accession: Genbank accession numbers

TABLE 2
FIG. 2 from BRCA 001 US
Table 2 shows genes downregulated in tumor tissue compared to normal breast tissue.

| Pkey | ExAccn | UnigeneID | Unigene Tittle | R1 |
|---|---|---|---|---|
| 100499 | T51986 | Hs.283108 | hemoglobin, gamma G | 10 |
| 100549 | BE142019 | Hs.222056 | Homo sapiens cDNA FLJ11572 fis, clone HE | 10 |
| 100654 | A03758 | | NM_000477*: Homo sapiens albumin (ALB), m | 10 |
| 100971 | BE379727 | Hs.83213 | fatty acid binding protein 4, adipocyte | 10 |
| 101184 | NM_001674 | Hs.460 | activating transcription factor 3 | 10 |
| 101336 | NM_006732 | Hs.75678 | FBJ murine osteosarcoma viral oncogene h | 10 |
| 101367 | X03350 | Hs.4 | alcohol dehydrogenase 1B (class I), beta | 10 |
| 101447 | M21305 | | gb: Human alpha satellite and satellite 3 | 10 |
| 101461 | N98569 | Hs.76422 | phospholipase A2, group IIA (platelets, | 10 |
| 101511 | M27826 | Hs.267319 | endogenous retroviral protease | 10 |
| 101736 | M74447 | Hs.502 | transporter 2, ATP-binding cassette, sub-famil | 10 |
| 102208 | U22961 | | gb: Human mRNA clone with similarity to L | 10 |
| 102450 | U48251 | Hs.75871 | protein kinase C binding protein 1 | 10 |
| 102800 | AA313538 | | gb: EST185419 Colon carcinoma (HCC) cell | 10 |
| 102857 | NM_006744 | Hs.76461 | retinol-binding protein 4, interstitial | 10 |
| 102990 | AA829286 | Hs.332053 | serum amyloid A1 | 10 |
| 103747 | AA081995 | | gb: zn26d06.r1 Stratagene neuroepithelium | 10 |
| 103812 | AA137107 | Hs.326391 | Homo sapiens, clone MGC: 16638, mRNA, com | 10 |
| 104093 | R50727 | Hs.336970 | ESTs | 10 |
| 104109 | AL353957 | Hs.284181 | hypothetical protein DKFZp434P0531 | 10 |
| 104250 | F06638 | Hs.12440 | Homo sapiens clone 24734 mRNA sequence | 10 |
| 104492 | N73185 | Hs.94285 | EST | 10 |
| 104506 | N91071 | Hs.109650 | ESTs | 10 |
| 104532 | AI498763 | Hs.203013 | hypothetical protein FLJ12748 | 10 |
| 104677 | AA009764 | Hs.190380 | ESTs | 10 |
| 104711 | AA017245 | Hs.32794 | ESTs | 10 |
| 104731 | AA019300 | Hs.125070 | ESTs, Moderately similar to I54374 gene | 10 |
| 105005 | AI298208 | Hs.28805 | ESTs | 10 |
| 105036 | AA130390 | Hs.25549 | hypothetical protein FLJ20898 | 10 |
| 105239 | AA221036 | | gb: zr03f12.r1 Stratagene NT2 neuronal pr | 10 |
| 106052 | N79885 | Hs.6382 | ESTs, Highly similar to T00391 hypotheti | 10 |
| 106181 | AI803651 | Hs.191608 | ESTs | 10 |
| 106283 | AI085846 | Hs.25522 | KIAA1808 protein | 10 |
| 106379 | AL042069 | Hs.119021 | DKFZP434N061 protein | 10 |
| 106451 | AW235928 | Hs.313182 | ESTs | 10 |
| 106491 | AA135688 | Hs.10083 | Homo sapiens, clone IMAGE: 4139786, mRNA, | 10 |
| 106782 | AW054886 | Hs.25682 | Homo sapiens mRNA for KIAA1863 protein, | 10 |
| 107124 | AB006532 | Hs.31442 | RecQ protein-like 4 | 10 |
| 107148 | AI005036 | Hs.334305 | GS1999full | 10 |
| 107214 | AF127026 | Hs.5394 | myosin IA | 10 |
| 107242 | AB020672 | Hs.175411 | KIAA0865 protein | 10 |
| 107331 | AI905985 | Hs.111805 | ESTs | 10 |
| 107447 | W28516 | Hs.19210 | hypothetical protein MGC11308 | 10 |
| 107451 | AL042425 | Hs.283976 | hypthetical protein PRO2389 | 10 |
| 107872 | BE271708 | Hs.95110 | ESTs, Weakly similar to A55943 1-phospha | 10 |
| 108351 | AA071193 | | gb: zf79b12.s1 Soares_pineal_gland_N3HPG | 10 |
| 109546 | F01449 | Hs.26954 | Homo sapiens mRNA; cDNA DKFZp762G123 (fr | 10 |
| 110433 | AW294162 | Hs.301062 | UDP-N-acetyl-alpha-D-galactosamine: polyp | 10 |
| 110976 | AL044174 | Hs.159526 | patched (Drosophila) homolog | 5 |
| 111168 | AI798376 | | gb: tr34b07.x1 NCI_CGAP_Ov23 Homo sapiens | 10 |
| 111651 | R16733 | Hs.20499 | ESTs | 10 |
| 111803 | AA593731 | Hs.325823 | ESTs, Moderately similar to ALU5_HUMAN A | 10 |
| 114484 | AA034378 | Hs.267319 | endogenous retroviral protease | 10 |
| 125284 | NM_002666 | Hs.103253 | perilipin | 10 |
| 128850 | AA193106 | Hs.180817 | chromosome 11 open reading frame 23 | 5 |
| 128903 | AW150717 | Hs.296176 | STAT induced STAT inhibitor 3 | 10 |
| 129346 | AF110141 | Hs.288908 | WAS protein family, member 2 | 10 |
| 129381 | AW245805 | Hs.110903 | claudin 5 (transmembrane protein deleted | 10 |
| 129516 | AF020038 | Hs.11223 | isocitrate dehydrogenase 1 (NADP+), solu | 10 |
| 129554 | BE222078 | Hs.113069 | ESTs | 10 |
| 130085 | M62402 | Hs.274313 | insulin-like growth factor binding prote | 10 |
| 130243 | D88435 | Hs.153227 | cyclin G associated kinase | 10 |
| 130400 | V00517 | Hs.283108 | hemoglobin, gamma G | 10 |
| 130436 | NM_001928 | Hs.155597 | D component of complement (adipsin) | 10 |
| 130563 | BE270472 | Hs.279900 | HSPC015 protein | 10 |
| 130589 | AL110226 | Hs.16441 | DKFZP434H204 protein | 10 |
| 130683 | AA993269 | Hs.17872 | Homo sapiens, clone IMAGE: 3875012, mRNA | 10 |
| 130689 | NM_006691 | Hs.17917 | extracellular link domain-containing 1 | 10 |
| 130689 | AA046747 | Hs.17917 | extracellular link domain-containing 1 | 10 |
| 130718 | N70196 | Hs.18376 | KIAA1319 protein | 10 |
| 130798 | M81349 | Hs.1955 | serum amyloid A4, constitutive | 10 |
| 130840 | BE048821 | Hs.20144 | small inducible cytokine subfamily A (Cy | 10 |
| 131184 | AB040935 | Hs.23954 | cerebral cell adhesion molecule | 10 |
| 131282 | X03350 | Hs.4 | alcohol dehydrogenase 1B (class I), beta | 10 |
| 131328 | AW939251 | Hs.25647 | v-fos FBJ murine osteosarcoma viral onco | 10 |
| 131543 | AW966881 | Hs.41639 | programmed cell death 2 | 10 |
| 131753 | AA829286 | Hs.332053 | serum amyloid A1 | 10 |
| 131785 | H69342 | Hs.26320 | TRABID protein | 10 |
| 131828 | AJ000263 | Hs.278658 | keratin, hair, basic, 6 (monilethrix) | 10 |
| 132426 | AW118072 | Hs.89981 | diacylglycerol kinase, zeta (104 kD) | 10 |
| 132675 | AI291496 | Hs.5476 | Homo sapiens, clone IMAGE: 3530123, mRNA, | 10 |
| 132898 | W28548 | Hs.224829 | ESTs | 10 |
| 132905 | NM_004235 | Hs.7934 | Kruppel-like factor 4 (gut) | 10 |
| 133120 | NM_003278 | Hs.65424 | tetranectin (plasminogen-binding protein | 10 |
| 133407 | AF017987 | Hs.7306 | secreted frizzled-related protein 1 | 10 |
| 133719 | H26904 | Hs.75736 | apolipoprotein D | 10 |
| 134007 | AF072441 | Hs.7840 | calcineurin binding protein 1 | 10 |
| 134055 | D86062 | Hs.182423 | ES1 (zebrafish) protein, human homolog o | 10 |
| 134111 | AI372588 | Hs.8022 | TU3A protein | 5 |
| 134117 | AA081846 | Hs.7921 | Homo sapiens mRNA; cDNA DKFZp566E183 (fr | 5 |
| 134177 | BE243319 | Hs.79672 | KIAA0652 gene product | 10 |
| 134369 | AF207664 | Hs.8230 | a disintegrin-like and metalloprotease ( | 10 |
| 134496 | M64936 | | gb: Homo sapiens retinoic acid-inducible | 10 |
| 134510 | NM_002757 | Hs.250870 | mitogen-activated protein kinase kinase | 10 |
| 134550 | M26315 | Hs.85258 | CD8 antigen, alpha polypeptide (p32) | 5 |
| 134758 | NM_000078 | Hs.89538 | cholesteryl ester transfer protein, plas | 5 |
| 134963 | NM_003394 | Hs.91985 | wingless-type MMTV integration site fami | 10 |
| 135066 | X04430 | Hs.93913 | interleukin 6 (interferon, beta 2) | 10 |
| 408790 | AW580227 | Hs.47860 | neurotrophic tyrosine kinase, receptor, type 2 | 10 |
| 446674 | AA563892 | Hs.306000 | solute carrier family 4 (anion exchanger), memb | 10 |

Pkey: Unique Eos probeset identifier number
ExAccn: Exemplar Accession number, Genbank accession number
UnigeneID: Unigene number
Unigene Title: Unigene gene title
R1: Ratio of normal breast tissue to tumor

TABLE 2A
Table 2A shows the accession numbers for those pkeys lacking unigeneID's for Table 2.
For each probeset, we have listed the gene cluster number from which the oligonucleotides
were designed. Gene clusters were compiled using sequences derived from Genbank ESTs
and mRNAs. These sequences were clustered based on sequence similarity using Clustering
and Alignment Tools (DoubleTwist, Oakland California). The Genbank accession numbers
for sequences comprising each cluster are listed in the “Accession” column.

| Pkey | CAT number | Accessions |
|---|---|---|
| 111168 | 38585_1 | AI798376 S46400 AW811617 AW811616 W00557 BE142245 AW858232 AW861851 AW858362 AA232351 AA218567 |
| | | AA055556 AW858231 AW857541 AW814172 H66214 AW814398 AF134164 AA243093 AA173345 AA199942 AA223384 |
| | | AA227092 AA227080 T12379 AA092174 T61139 AA149776 |
| | | AA699829 AW879188 AW813567 AW813538 AI267168 AA157718 |
| | | AA157719 AA100472 AA100774 AA130756 AA157705 AA157730 AA157715 AA053524 AW849581 AW854566 C05254 |
| | | AW882836 T92637 AW812621 AA206583 AA209204 BE156909 |
| | | AA226824 AI829309 AW991957 N66951 AA527374 H66215 |
| | | AA045564 AI694265 H60808 AA149726 AW195620 BE081333 BE073424 AW817662 AW817705 AW817703 AW817659 |
| | | BE081531 H59570 |
| 103747 | 117944_1 | AA081995 AA101099 |
| 134496 | 46501_1 | M64936 AI025512 AI382987 BE061777 AA089966 BE169930 T41176 AW594624 BE502415 AA121893 AI269283 T40311 |
| | | AI684569 AA257011 AI079277 AI241318 BE327710 AW975215 AW896268 AA884990 BE327514 |
| 105239 | 34624_1 | AA221036 R87170 BE537068 BE544757 C18935 AW812058 T92565 AA227415 AA233942 AA223237 AA668403 AA601627 |
| | | AW869639 BE061833 BE000620 AW961170 AW847519 AA308542 AW821833 AW945688 C04699 AA205504 AA377241 |
| | | AW821667 AA055720 AW817981 AW856468 AA155719 AA179928 T03007 AW754298 AA227407 AA113928 AA307904 |
| | | C16859 |
| 100654 | tigr_{—} | A03758 A06977 A15293 D17029 D17107 D17171 |
| | | L00132 L00133 M12523 M13075 M13076 M92816 U22961 V00494 V00495 |
| | HT2969 | X51363 X51364 X51365 |
| 102208 | 6735_9 | U22961 AA203623 AA503337 AI174733 AI192802 C06092 AA035357 AI190619 AI199244 AI828450 AA602296 AI378195 |
| | | AI209170 AI186653 AI127795 AI183846 H77389 AI589465 AA629390 H94306 AI018388 R68584 AA027196 AI745413 |
| | | AI685092 AI093426 AI623873 AI074570 N50096 AA047486 N25060 AA327614 AI042512 AI383957 AA156873 AI333101 |
| | | N70806 AI141254 AI383191 AI401237 AI080709 AI093400 W84549 |
| | | T90806 R00012 W01413 AA630557 AI378348 AI559265 |
| | | AA877103 W84464 AA625146 R68379 AI133207 AI132980 AI133214 AI064826 AI061615 AI133473 AI174852 AI133404 |
| | | AI133272 V00494 M12523 M12523 AI207526 AI133120 AI064802 |
| | | AI174993 AI114729 AI061645 AI064716 AI064959 H77388 |
| | | T85706 AF075298 AI110799 D17107 NM_000477 AF190168 R50724 AI248416 AI207432 AI133684 AI133345 AI174710 |
| | | AI133290 AI133304 AI174948 AI207484 AI110717 AF074624 AI114515 AF063516 AI110642 AI114559 AI114498 AI114759 |
| | | AI207568 AI064960 AI174753 AI114666 R69184 R00011 AI064997 T60501 AI207701 T71735 AA385318 H73569 T60496 |
| | | H94399 AI133158 T74675 AA484750 T73413 T55909 R50261 |
| | | T72061 N80533 T51189 T74936 AI207490 AI132925 AI064701 |
| | | AI174748 AI114663 AI133104 AI132999 AI133100 AI064925 AI064979 AI133063 AA343347 T69091 AA233989 T39772 |
| | | AI444620 T52290 D16931 T40012 T48403 T58926 T69195 AI133061 T50850 AI400677 AI091136 AA334608 T57411 Z20979 |
| | | N56507 T87485 AI133622 AA343370 T40075 T69671 T53849 T74820 AF075316 AI110818 T40121 T57381 AI114468 |
| | | AA332728 T51362 AI114589 R06691 AI110629 AF063503 AI140543 AA334661 AA332720 AA343262 T73513 T86549 |
| | | AI114840 T57284 T39981 T61407 T72757 T51749 T56630 AA343125 T72126 R94135 T83028 T39972 T39896 AI174786 |
| | | AI132926 R09237 AI064838 AI133660 T60398 T88753 T55930 T92126 AI444602 T60996 AI114792 H93911 AI133106 |
| | | R10779 AI065020 T90925 T50889 D17029 AI133703 AA333805 |
| | | AI133040 AI133017 AI064857 AI110730 AF074637 AI207567 |
| | | H71080 T73217 AA343950 AI174743 AA334224 AA334281 R06692 T64739 T40163 T60628 T81661 T73179 R01842 |
| | | AA501730 T39931 T39662 T40136 AA334904 T71425 H77784 R00874 AI065049 T84512 T55918 AI207595 T39951 |
| | | AA005016 T60361 T69176 T73356 T58795 T61233 T39955 T60612 AI114676 AI064778 AA035710 W52763 AI114786 |
| | | T83564 AA341859 T81684 T55769 AI114710 T51776 AA343213 AI114714 T58102 AI110809 R28984 AI174854 AA305675 |
| | | AA343592 T53836 T46869 T64721 T55508 W05241 T54019 T57945 T60513 T48364 AF075308 W86731 T82851 T48269 |
| | | H54053 T73211 AI114590 T48317 T55965 T74857 R84226 T56552 T52231 T74946 T76976 R02576 T95666 AI203974 |
| | | AI189471 AA005147 AI478102 AI207662 AI192792 AI768421 |
| | | AI064737 AW051713 AA936693 AI133117 AI766232 AI913646 |
| | | T83962 AI065112 AI207689 AI174684 AI207702 T81475 AI133325 AI032512 AA701169 AI936354 AI114720 AI433289 |
| | | AA046980 AI823482 AI114536 AA860651 AW242644 R07469 |
| | | AW300438 AI133416 AW271670 AI991363 T78943 AI823481 |
| | | AA845518 AA719124 AA883454 T68850 T69115 AI935509 AI150977 T62890 T71374 T68294 AI174774 T67411 T68318 |
| | | AI064689 T56624 T69010 T68982 T68302 AI332829 T72908 AI064819 AI205880 T62895 T69430 T95111 AA025050 T73330 |
| | | W52657 T71984 T69118 W92684 AI114860 T62093 T61797 AI522333 T73322 H92981 T56018 T61811 T57232 AI336158 |
| | | T61821 T69457 T62900 T62912 T72917 T46885 AI702448 T57212 T57203 R94581 T71311 T61819 T89358 T67708 T70918 |
| | | T59166 AI187111 T64308 T62071 T69427 AI114750 T60430 R09734 T69033 T69141 T69453 T67908 R16809 T69394 |
| | | AI207729 T55839 T90273 T73339 AW194909 T75486 T71850 T71305 T71287 T53877 T73452 T68852 N75290 AI312890 |
| | | T67751 AI174983 T51679 T54851 H69880 N73734 AA443453 T73466 H69672 N53869 T68447 D11809 D12412 T64300 |
| | | T28321 T55864 |
| 102800 | 14782_20 | AA313538 U88895 U88902 |
| 108351 | genbank_{—} | AA071193 |
| | AA071193 | |
| 101447 | entrez_{—} | M21305 |
| | M21305 | |

Pkey: Unique Eos probeset identifier number
CAT number: Gene cluster number
Accession: Genbank accession numbers

TABLE 3
FIG. 3 from BRCA 001 US
Table 3 shows genes downregulated in tumor tissue compared to normal breast tissue.

| Pkey | ExAccn | UnigeneID | UnigeneTitle | R1 |
|---|---|---|---|---|
| 101336 | NM_006732 | Hs.75678 | FBJ murine osteosarcoma viral oncogene h | 10.0 |
| 102208 | U22961 | | gb: Human mRNA clone with similarity to L | 10.0 |
| 102990 | AA829286 | Hs.332053 | serum amyloid A1 | 10.0 |
| 111168 | AI798376 | | gb: tr34b07.x1 NCI_CGAP_Ov23 Homo sapiens | 10.0 |
| 111803 | AA593731 | Hs.325823 | ESTs, Moderately similar to ALU5_HUMAN A | 10.0 |
| 130085 | M62402 | Hs.274313 | insulin-like growth factor binding prote | 10.0 |
| 130840 | BE048821 | Hs.20144 | small inducible cytokine subfamily A (Cy | 10.0 |
| 131543 | AW966881 | Hs.41639 | programmed cell death 2 | 10.0 |
| 133120 | NM_003278 | Hs.65424 | tetranectin (plasminogen-binding protein | 10.0 |
| 134758 | NM_000078 | Hs.89538 | cholesteryl ester transfer protein, plas | 10.0 |

Pkey: Unique Eos probeset identifier number
ExAccn: Exemplar Accession number, Genbank accession number
UnigeneID: Unigene number
Unigene Title: Unigene gene title
R1: Ratio of normal breast tissue to tumor

TABLE 3A
Table 3A shows the accession numbers for those pkeys lacking unigeneID's for Table 3. For
each probeset, we have listed the gene cluster number from which the oligonucleotides were
designed. Gene clusters were compiled using sequences derived from Genbank ESTs and
mRNAs. These sequences were clustered based on sequence similarity using Clustering and
Alignment Tools (DoubleTwist, Oakland California). The Genbank accession numbers for
sequences comprising each cluster are listed in the “Accession” column.

| | CAT | |
|---|---|---|
| Pkey | number | Accessions |
| 111168 | 38585_1 | AI798376 S46400 AW811617 AW811616 W00557 BE142245 AW858232 AW861851 AW858362 AA232351 AA218567 |
| | | AA055556 AW858231 AW857541 AW814172 H66214 AW814398 AF134164 AA243093 AA173345 AA199942 AA223384 |
| | | AA227092 AA227080 T12379 AA092174 T61139 AA149776 |
| | | AA699829 AW879188 AW813567 AW813538 AI267168 AA157718 |
| | | AA157719 AA100472 AA100774 AA130756 AA157705 AA157730 AA157715 AA053524 AW849581 AW854566 C05254 |
| | | AW882836 T92637 AW812621 AA206583 AA209204 BE156909 AA226824 AI829309 AW991957 N66951 AA527374 H66215 |
| | | AA045564 AI694265 H60808 AA149726 AW195620 BE081333 BE073424 AW817662 AW817705 AW817703 AW817659 |
| | | BE081531 H59570 |
| 102208 | 6735_9 | U22961 AA203623 AA503337 AI174733 AI192802 C06092 AA035357 AI190619 AI199244 AI828450 AA602296 AI378195 |
| | | AI209170 AI186653 AI127795 AI183846 H77389 AI589465 |
| | | AA629390 H94306 AI018388 R68584 AA027196 AI745413 AI685092 |
| | | AI093426 AI623873 AI074570 N50096 AA047486 N25060 |
| | | AA327614 AI042512 AI383957 AA156873 AI333101 N70806 AI141254 |
| | | AI383191 AI401237 AI080709 AI093400 W84549 T90806 R00012 W01413 AA630557 AI378348 AI559265 AA877103 W84464 |
| | | AA625146 R68379 AI133207 AI132980 AI133214 AI064826 |
| | | AI061615 AI133473 AI174852 AI133404 AI133272 V00494 M12523 |
| | | M12523 AI207526 AI133120 AI064802 AI174993 AI114729 |
| | | AI061645 AI064716 AI064959 H77388 T85706 AF075298 AI110799 |
| | | D17107 NM_000477 AF190168 R50724 AI248416 AI207432 AI133684 AI133345 AI174710 AI133290 AI133304 AI174948 |
| | | AI207484 AI110717 AF074624 AI114515 AF063516 AI110642 AI114559 AI114498 AI114759 AI207568 AI064960 AI174753 |
| | | AI114666 R69184 R00011 AI064997 T60501 AI207701 |
| | | T71735 AA385318 H73569 T60496 H94399 AI133158 T74675 AA484750 |
| | | T73413 T55909 R50261 T72061 N80533 T51189 T74936 AI207490 |
| | | AI132925 AI064701 AI174748 AI114663 AI133104 AI132999 |
| | | AI133100 AI064925 AI064979 AI133063 AA343347 T69091 AA233989 T39772 AI444620 T52290 D16931 T40012 T48403 |
| | | T58926 T69195 AI133061 T50850 AI400677 AI091136 |
| | | AA334608 T57411 Z20979 N56507 T87485 AI133622 AA343370 T40075 |
| | | T69671 T53849 T74820 AF075316 AI110818 T40121 T57381 AI114468 AA332728 T51362 AI114589 R06691 AI110629 |
| | | AF063503 AI140543 AA334661 AA332720 AA343262 T73513 T86549 AI114840 T57284 T39981 T61407 T72757 T51749 |
| | | T56630 AA343125 T72126 R94135 T83028 T39972 T39896 AI174786 AI132926 R09237 AI064838 AI133660 T60398 T88753 |
| | | T55930 T92126 AI444602 T60996 AI114792 H93911 |
| | | AI133106 R10779 AI065020 T90925 T50889 D17029 AI133703 AA333805 |
| | | AI133040 AI133017 AI064857 AI110730 AF074637 AI207567 H71080 T73217 AA343950 AI174743 AA334224 AA334281 |
| | | R06692 T64739 T40163 T60628 T81661 T73179 R01842 |
| | | AA501730 T39931 T39662 T40136 AA334904 T71425 H77784 R00874 |
| | | AI065049 T84512 T55918 AI207595 T39951 AA005016 T60361 T69176 T73356 T58795 T61233 T39955 T60612 AI114676 |
| | | AI064778 AA035710 W52763 AI114786 T83564 AA341859 T81684 T55769 AI114710 T51776 AA343213 AI114714 T58102 |
| | | AI110809 R28984 AI174854 AA305675 AA343592 T53836 T46869 T64721 T55508 W05241 T54019 T57945 T60513 T48364 |
| | | AF075308 W86731 T82851 T48269 H54053 T73211 AI114590 T48317 T55965 T74857 R84226 T56552 T52231 T74946 T76976 |
| | | R02576 T95666 AI203974 AI189471 AA005147 AI478102 AI207662 AI192792 AI768421 AI064737 AW051713 AA936693 |
| | | AI133117 AI766232 AI913646 T83962 AI065112 AI207689 |
| | | AI174684 AI207702 T81475 AI133325 AI032512 AA701169 AI936354 |
| | | AI114720 AI433289 AA046980 AI823482 AI114536 AA860651 |
| | | AW242644 R07469 AW300438 AI133416 AW271670 AI991363 |
| | | T78943 AI823481 AA845518 AA719124 AA883454 T68850 T69115 AI935509 AI150977 T62890 T71374 T68294 AI174774 |
| | | T67411 T68318 AI064689 T56624 T69010 T68982 T68302 AI332829 T72908 AI064819 AI205880 T62895 T69430 T95111 |
| | | AA025050 T73330 W52657 T71984 T69118 W92684 AI114860 T62093 T61797 AI522333 T73322 H92981 T56018 T61811 |
| | | T57232 AI336158 T61821 T69457 T62900 T62912 T72917 T46885 AI702448 T57212 T57203 R94581 T71311 T61819 T89358 |
| | | T67708 T70918 T59166 AI187111 T64308 T62071 T69427 AI114750 T60430 R09734 T69033 T69141 T69453 T67908 R16809 |
| | | T69394 AI207729 T55839 T90273 T73339 AW194909 T75486 T71850 T71305 T71287 T53877 T73452 T68852 N75290 |
| | | AI312890 T67751 AI174983 T51679 T54851 H69880 N73734 AA443453 T73466 H69672 N53869 T68447 D11809 D12412 |
| | | T64300 T28321 T55864 |

Pkey: Unique Eos probeset identifier number
CAT number: Gene cluster number
Accession: Genbank accession numbers

TABLE 4
FIG. 4 from BRCA 001 US
Table 4 shows genes upregulated in tumor tissue compared to normal breast tissue.

| Pkey | ExAccn | UnigeneID | Unigene Title | R1 |
|---|---|---|---|---|
| 100113 | NM_001269 | Hs.84746 | chromosome condensation 1 | 2.3 |
| 100114 | X02308 | Hs.82962 | thymidylate synthetase | 2.9 |
| 100131 | D12485 | Hs.11951 | ectonucleotide pyrophosphatase/phosphodiesterase 1 | 1.9 |
| 100146 | BE185499 | Hs.2471 | KIAA0020 gene product | 1.9 |
| 100147 | D13666 | Hs.136348 | osteoblast specific factor 2 (fasciclin I-like) (periostin) | 7.5 |
| 100154 | H60720 | Hs.81892 | KIAA0101 gene product | 9.2 |
| 100163 | W44671 | Hs.124 | gene predicted from cDNA with a complete coding sequence | 1.6 |
| 100220 | AW015534 | Hs.217493 | annexin A2 | 2.0 |
| 100265 | D38521 | Hs.112396 | KIAA0077 protein | 1.5 |
| 100271 | BE160081 | Hs.256290 | S100 calcium-binding protein A11 (calgizzarin) | 13.5 |
| 100275 | BE242802 | Hs.154797 | KIAA0090 protein | 5.1 |
| 100323 | D50920 | Hs.23106 | KIAA0130 gene product | 1.9 |
| 100335 | AW247529 | Hs.6793 | platelet-activating factor acetylhydrolase, isoform lb, gamma subunit (29 kD) | 2.7 |
| 100364 | NM_004341 | Hs.154868 | carbamoyl-phosphate synthetase 2, aspartate transcarbamylase, and dihydroorotase | 2.0 |
| 100372 | NM_014791 | Hs.184339 | KIAA0175 gene product | 2.6 |
| 100393 | D84145 | Hs.39913 | novel RGD-containing protein | 3.2 |
| 100400 | AW954324 | Hs.75790 | phosphatidylinositol glycan, class C | 1.5 |
| 100418 | D86978 | Hs.84790 | KIAA0225 protein | 2.0 |
| 100482 | M65028 | Hs.81361 | heterogeneous nuclear ribonucleoprotein A/B | 2.9 |
| 100518 | NM_004415 | Hs.74316 | desmoplakin (DPI, DPII) | 1.9 |
| 100666 | L05424 | Hs.169610 | CD44 antigen (homing function and Indian blood group system) | 5.7 |
| 100667 | L05424 | Hs.169610 | CD44 antigen (homing function and Indian blood group system) | 9.0 |
| 100668 | L05424 | Hs.169610 | CD44 antigen (homing function and Indian blood group system) | 7.6 |
| 100678 | AW502935 | Hs.740 | PTK2 protein tyrosine kinase 2 | 53.2 |
| 100685 | AA328229 | Hs.184582 | ribosomal protein L24 | 1.8 |
| 100690 | AA383256 | Hs.1657 | estrogen receptor 1 | 1.6 |
| 100783 | AF078847 | Hs.191356 | general transcription factor IIH, polypeptide 2 (44 kD subunit) | 5.9 |
| 100850 | AA836472 | Hs.297939 | cathepsin B | 1.7 |
| 100892 | BE245294 | Hs.180789 | S164 protein | 1.7 |
| 100945 | AF002225 | Hs.180686 | ubiquitin protein ligase E3A (human papilloma virus E6-associated protein, Angelman syndrome) | 1.5 |
| 100969 | AA157634 | Hs.79172 | solute carrier family 25 (mitochondrial carrier; adenine nucleotide translocator), member 5 | 6.3 |
| 100988 | AK000405 | Hs.76480 | ubiquitin-like 4 | 11.4 |
| 100999 | H38765 | Hs.80706 | diaphorase (NADH/NADPH) (cytochrome b-5 reductase) | 1.6 |
| 101031 | J05070 | Hs.151738 | matrix metalloproteinase 9 (gelatinase B, 92 kD gelatinase, 92 kD type IV collagenase) | 8.2 |
| 101045 | J05614 | | gb: Human proliferating cell nuclear antigen (PCNA) gene, promoter region. | 5.0 |
| 101077 | N99692 | Hs.75227 | Empirically selected from AFFX single probeset | 2.6 |
| 101093 | L06419 | Hs.75093 | procollagen-lysine, 2-oxoglutarate 5-dioxygenase (lysine hydroxylase, Ehlers-Danlos syndrome type VI) | 1.4 |
| 101161 | NM_006262 | Hs.37044 | peripherin | 16.9 |
| 101186 | AA020956 | Hs.179881 | core-binding factor, beta subunit | 2.0 |
| 101216 | AA284166 | Hs.84113 | cyclin-dependent kinase inhibitor 3 (CDK2-associated dual specificity phosphatase) | 1.8 |
| 101228 | AA333387 | Hs.82916 | chaperonin containing TCP1, subunit 6A (zeta 1) | 1.7 |
| 101247 | AA132666 | Hs.78802 | glycogen synthase kinase 3 beta | 1.9 |
| 101249 | L18964 | Hs.1904 | protein kinase C, iota | 1.5 |
| 101332 | J04088 | Hs.156346 | topoisomerase (DNA) II alpha (170 kD) | 5.2 |
| 101332 | J04088 | Hs.156346 | topoisomerase (DNA) II alpha (170 kD) | 3.4 |
| 101352 | AI494299 | Hs.16297 | COX17 (yeast) homolog, cytochrome c oxidase assembly protein | 6.3 |
| 101396 | BE267931 | Hs.78996 | proliferating cell nuclear antigen | 4.2 |
| 101445 | M21259 | | gb: Human Alu repeats in the region 5′ to the small nuclear rib | 1.9 |
| 101470 | NM_000546 | Hs.1846 | tumor protein p53 (Li-Fraumeni syndrome) | 1.6 |
| 101478 | NM_002890 | Hs.758 | RAS p21 protein activator (GTPase activating protein) 1 | 2.5 |
| 101483 | M24486 | Hs.76768 | procollagen-proline, 2-oxoglutarate 4-dioxygenase (proline 4-hydroxylase), alpha polypeptide I | 5.5 |
| 101540 | J04977 | Hs.84981 | X-ray repair complementing defective repair in Chinese hamster cells 5 (double-strand-break rejoining | 2.1 |
| 101573 | AW248421 | Hs.250758 | proteasome (prosome, macropain) 26S subunit, ATPase, 3 | 1.6 |
| 101580 | NM_012151 | Hs.83363 | coagulation factor VIII-associated (intronic transcript) | 5.7 |
| 101592 | AF064853 | Hs.91299 | guanine nucleotide binding protein (G protein), beta polypeptide 2 | 1.8 |
| 101592 | AF064853 | Hs.91299 | guanine nucleotide binding protein (G pr | 5.6 |
| 101621 | BE391804 | Hs.62661 | guanylate binding protein 1, interferon-inducible, 67 kD | 2.4 |
| 101702 | AW504089 | Hs.179574 | protein phosphatase 2 (formerly 2A), regulatory subunit B (PR 52), alpha isoform | 1.3 |
| 101734 | M74099 | Hs.147049 | cut (Drosophila)-like 1 (CCAAT displacement protein) | 2.1 |
| 101759 | M80244 | Hs.184601 | solute carrier family 7 (cationic amino acid transporter, y + system), member 5 | 5.0 |
| 101767 | M81057 | Hs.180884 | carboxypeptidase B1 (tissue) | 14.4 |
| 101782 | AA306495 | Hs.1869 | phosphoglucomutase 1 | 5.2 |
| 101805 | AW409747 | Hs.75612 | stress-induced-phosphoprotein 1 (Hsp70/Hsp90-organizing protein) | 8.6 |
| 101806 | AA586894 | Hs.112408 | S100 calcium-binding protein A7 (psoriasin 1) | 8.9 |
| 101810 | NM_000318 | Hs.180612 | peroxisomal membrane protein 3 (35 kD, Zellweger syndrome) | 3.2 |
| 101879 | AA176374 | Hs.243886 | nuclear autoantigenic sperm protein (histone-binding) | 1.6 |
| 101911 | AA441787 | Hs.119689 | glycoprotein hormones, alpha polypeptide | 31.3 |
| 101920 | AF182645 | Hs.8024 | IK cytokine, down-regulator of HLA II | 1.8 |
| 101973 | U41514 | Hs.80120 | UDP-N-acetyl-alpha-D-galactosamine: polypeptide N-acetylgalactosaminyltransferase 1 (GalNAc-T1) | 2.4 |
| 101983 | AI904232 | Hs.75323 | prohibitin | 8.4 |
| 102009 | BE245149 | Hs.82643 | protein tyrosine kinase 9 | 1.3 |
| 102036 | BE250127 | Hs.82906 | CDC20 (cell division cycle 20, S. cerevisiae, homolog) | 2.0 |
| 102083 | T35901 | Hs.75117 | interleukin enhancer binding factor 2, 45 kD | 1.6 |
| 102083 | T35901 | Hs.75117 | interleukin enhancer binding factor 2, 4 | 1.3 |
| 102107 | BE258602 | Hs.182366 | heat shock protein 75 | 1.4 |
| 102123 | NM_001809 | Hs.1594 | centromere protein A (17 kD) | 1.8 |
| 102165 | BE313280 | Hs.159627 | death associated protein 3 | 4.6 |
| 102198 | AW950852 | Hs.74598 | polymerase (DNA directed), delta 2, regulatory subunit (50 kD) | 4.3 |
| 102217 | AA829978 | Hs.301613 | JTV1 gene | 6.7 |
| 102220 | U24389 | Hs.65436 | lysosomal | 4.3 |
| 102234 | AW163390 | Hs.278554 | heterochromatin-like protein 1 | 1.9 |
| 102260 | AL039104 | Hs.159557 | karyopherin alpha 2 (RAG cohort 1, importin alpha 1) | 4.4 |
| 102302 | AA306342 | Hs.69171 | protein kinase C-like 2 | 2.7 |
| 102330 | BE298063 | Hs.77254 | chromobox homolog 1 (Drosophila HP1 beta) | 1.5 |
| 102339 | BE378432 | Hs.95577 | cyclin-dependent kinase 4 | 2.3 |
| 102348 | U37519 | Hs.87539 | aldehyde dehydrogenase 3 family, member B2 | 2.0 |
| 102349 | AU077055 | Hs.289107 | baculoviral IAP repeat-containing 2 | 3.2 |
| 102369 | U39840 | Hs.299867 | hepatocyte nuclear factor 3, alpha | 2.0 |
| 102374 | U33635 | Hs.90572 | PTK7 protein tyrosine kinase 7 | 6.2 |
| 102391 | AA296874 | Hs.77494 | deoxyguanosine kinase | 1.5 |
| 102455 | U48705 | Hs.75562 | discoidin domain receptor family, member 1 | 6.9 |
| 102465 | NM_001359 | Hs.81548 | 2,4-dienoyl CoA reductase 1, mitochondrial | 1.8 |
| 102488 | U50939 | Hs.61828 | amyloid beta precursor protein-binding protein 1, 59 kD | 1.5 |
| 102489 | AL080116 | Hs.74420 | origin recognition complex, subunit 3 (yeast homolog)-like | 3.3 |
| 102494 | AI188137 | Hs.75193 | COP9 homolog | 2.1 |
| 102501 | AF217197 | Hs.74562 | siah binding protein 1; FBP interacting repressor; pyrimidine tract binding splicing | 3.2 |
| 102522 | BE250944 | Hs.183556 | solute carrier family 1 (neutral amino acid transporter), member 5 | 2.8 |
| 102532 | AF040253 | Hs.70186 | suppressor of Ty (S. cerevisiae) 5 homolog | 5.7 |
| 102564 | U59423 | Hs.79067 | MAD (mothers against decapentaplegic, Drosophila) homolog 1 | 2.3 |
| 102568 | W81489 | Hs.223025 | RAB31, member RAS oncogene family | 5.3 |
| 102580 | U60808 | Hs.152981 | CDP-diacylglycerol synthase (phosphatidate cytidylyltransferase) 1 | 2.1 |
| 102581 | AU077228 | Hs.77256 | enhancer of zeste (Drosophila) homolog 2 | 1.6 |
| 102582 | U61232 | Hs.32675 | tubulin-specific chaperone e | 2.1 |
| 102617 | AW161453 | Hs.198767 | COP9 (constitutive photomorphogenic, Arabidopsis, homolog) subunit 5 | 1.8 |
| 102618 | AL037672 | Hs.81071 | extracellular matrix protein 1 | 5.8 |
| 102627 | AL021918 | Hs.158174 | zinc finger protein 184 (Kruppel-like) | 1.3 |
| 102663 | NM_002270 | Hs.168075 | karyopherin (importin) beta 2 | 1.8 |
| 102676 | BE262989 | Hs.12045 | putative protein | 2.3 |
| 102687 | NM_007019 | Hs.93002 | ubiquitin carrier protein E2-C | 4.3 |
| 102689 | U96132 | Hs.171280 | hydroxyacyl-Coenzyme A dehydrogenase, type II | 6.0 |
| 102696 | BE540274 | Hs.239 | forkhead box M1 | 4.2 |
| 102704 | AU077058 | Hs.54089 | BRCA1 associated RING domain 1 | 1.9 |
| 102705 | T97490 | Hs.50002 | small inducible cytokine subfamily A (Cys-Cys), member 19 | 2.3 |
| 102750 | AB014460 | Hs.66196 | nth (E. coli endonuclease III)-like 1 | 1.2 |
| 102801 | BE252241 | Hs.38041 | pyridoxal (pyridoxine, vitamin B6) kinase | 6.4 |
| 102812 | U90549 | Hs.236774 | high-mobility group (nonhistone chromosomal) protein 17-like 3 | 1.6 |
| 102827 | BE244588 | Hs.6456 | chaperonin containing TCP1, subunit 2 (beta) | 5.6 |
| 102831 | AA262170 | Hs.80917 | adaptor-related protein complex 3, sigma 1 subunit | 2.0 |
| 102844 | AV653790 | Hs.324275 | WW domain-containing protein 1 | 1.3 |
| 102868 | X02419 | Hs.77274 | plasminogen activator, urokinase | 4.4 |
| 102925 | BE440142 | Hs.2943 | signal recognition particle 19 kD | 1.9 |
| 102935 | BE561850 | Hs.80506 | small nuclear ribonucleoprotein polypeptide A′ | 2.4 |
| 102968 | AU076611 | Hs.154672 | methylene tetrahydrofolate dehydrogenase (NAD + dependent), methenyltetrahydrofolate cyclohydrolase | 2.7 |
| 102983 | BE387202 | Hs.118638 | non-metastatic cells 1, protein (NM23A) expressed in | 3.1 |
| 102985 | U95742 | Hs.2707 | G1 to S phase transition 1 | 5.2 |
| 103023 | AW500470 | Hs.117950 | multifunctional polypeptide similar to SAICAR synthetase and AIR carboxylase | 1.6 |
| 103038 | AA926960 | Hs.334883 | CDC28 protein kinase 1 | 2.5 |
| 103060 | NM_005940 | Hs.155324 | matrix metalloproteinase 11 (MMP11; stromelysin 3) | 4.5 |
| 103080 | AU077231 | Hs.82932 | cyclin D1 (PRAD1: parathyroid adenomatosis 1) | 3.1 |
| 103089 | D31152 | Hs.179729 | collagen, type X, alpha 1 (Schmid metaphyseal chondrodysplasia) | 2.4 |
| 103177 | BE244377 | Hs.48876 | famesyl-diphosphate famesyltransferase 1 | 3.5 |
| 103178 | AA205475 | Hs.275865 | ribosomal protein S18 | 9.9 |
| 103179 | NM_001777 | Hs.82685 | CD47 antigen (Rh-related antigen, integrin-associated signal transducer) | 1.3 |
| 103181 | X69636 | Hs.334731 | Homo sapiens, clone IMAGE: 3448306, mRNA, partial cds | 2.0 |
| 103185 | NM_006825 | Hs.74368 | transmembrane protein (63 kD), endoplasmic reticulum/Golgi intermediate compartment | 1.6 |
| 103191 | AA401039 | Hs.2903 | protein phosphatase 4 (formerly X), catalytic subunit | 2.5 |
| 103193 | NM_004766 | Hs.75724 | coatomer protein complex, subunit beta 2 (beta prime) | 2.2 |
| 103194 | NM_004939 | Hs.78580 | DEAD/H (Asp-Glu-Ala-Asp/His) box polypeptide 1 | 6.3 |
| 103206 | X72755 | Hs.77367 | monokine induced by gamma interferon | 8.8 |
| 103223 | BE275607 | Hs.1708 | chaperonin containing TCP1, subunit 3 (gamma) | 3.0 |
| 103232 | X75962 | Hs.129780 | tumor necrosis factor receptor superfamily, member 4 | 1.8 |
| 103238 | AI369285 | Hs.75189 | death-associated protein | 5.6 |
| 103297 | NM_001545 | Hs.9078 | immature colon carcinoma transcript 1 | 1.9 |
| 103330 | AI803447 | Hs.77496 | small nuclear ribonucleoprotein polypeptide G | 2.5 |
| 103349 | X89059 | | gb: H. sapiens mRNA for unknown protein expressed in macrophage | 1.6 |
| 103376 | AL036166 | Hs.323378 | coated vesicle membrane protein | 1.8 |
| 103391 | X94453 | Hs.114366 | pyrroline-5-carboxylate synthetase (glutamate gamma-semialdehyde synthetase) | 2.3 |
| 103392 | X94563 | | gb: H. sapiens dbi/acbp gene exon 1 & 2. | 4.0 |
| 103430 | BE564090 | Hs.20716 | translocase of inner mitochondrial membrane 17 (yeast) homolog A | 1.3 |
| 103491 | AF264750 | Hs.288971 | myeloid/lymphoid or mixed-lineage leukemia 3 | 5.6 |
| 103505 | AL031224 | Hs.33102 | transcription factor AP-2 beta (activating enhancer-binding protein 2 beta) | 5.1 |
| 103547 | AI376722 | Hs.180062 | proteasome (prosome, macropain) subunit, beta type, 8 (large multifunctional protease 7) | 9.7 |
| 103588 | NM_006218 | Hs.85701 | phosphoinositide-3-kinase, catalytic, alpha polypeptide | 2.0 |
| 103613 | NM_000346 | Hs.2316 | SRY (sex determining region Y)-box 9 (campomelic dysplasia, autosomal sex-reversal) | 1.3 |
| 103621 | BE379766 | Hs.150675 | polymerase (RNA) II (DNA directed) polypeptide K (7.0 kD) | 2.0 |
| 103622 | AA609685 | Hs.278672 | membrane component, chromosome 11, surface marker 1 | 2.3 |
| 103727 | AI878883 | Hs.296381 | growth factor receptor-bound protein 2 | 1.3 |
| 103749 | AL135301 | Hs.8768 | hypothetical protein FLJ10849 | 1.8 |
| 103754 | AI015709 | Hs.172089 | Homo sapiens mRNA; cDNA DKFZp586I2022 (from clone DKFZp586I2022) | 1.3 |
| 103780 | AA094752 | Hs.169992 | hypothetical 43.2 kD protein | 7.5 |
| 103795 | H26531 | Hs.7367 | Homo sapiens BTB domain protein (BDPL) mRNA, partial cds | 1.2 |
| 103797 | AA080912 | | gb: zn04d03.r1 Stratagene hNT neuron (937233) Homo sapiens cDNA clone 5′ similar | 1.5 |
| 103813 | AI042582 | Hs.181271 | CGI-120 protein | 1.5 |
| 103855 | W02363 | Hs.302267 | hypothetical protein FLJ10330 | 1.5 |
| 103886 | AK001278 | Hs.105737 | hypothetical protein FLJ10416 similar to constitutive photomorphogenic protein 1 | 6.5 |
| 104052 | NM_002407 | Hs.97644 | mammaglobin 2 | 2.9 |
| 104079 | AA251242 | Hs.103238 | ESTs | 1.4 |
| 104174 | AA478984 | Hs.6451 | PRO0659 protein | 5.6 |
| 104227 | AB002343 | Hs.98938 | protocadherin alpha 9 | 1.6 |
| 104275 | AI751970 | Hs.101067 | GCN5 (general control of amino-acid synthesis, yeast homolog)-like 2 | 5.4 |
| 104325 | BE379766 | Hs.150675 | polymerase (RNA) II (DNA directed) polypeptide K (7.0 kD) | 6.3 |
| 104370 | AA324597 | Hs.21851 | Homo sapiens cDNA FLJ12900 fis, clone NT2RP2004321 | 1.6 |
| 104423 | R83113 | Hs.1432 | protein kinase C substrate 80K-H | 5.2 |
| 104482 | AB037762 | Hs.44268 | myelin gene expression factor 2 | 1.2 |
| 104532 | AI498763 | Hs.203013 | hypothetical protein FLJ12748 | 2.1 |
| 104563 | AL117403 | Hs.306189 | DKFZP434F1735 protein | 1.2 |
| 104667 | AI239923 | Hs.30098 | ESTs | 1.3 |
| 104757 | AI694413 | Hs.332649 | olfactory receptor, family 2, subfamily I, member 6 | 2.3 |
| 104804 | AI858702 | Hs.31803 | ESTs, Weakly similar to N-WASP [H. sapiens] | 1.3 |
| 104806 | AB023175 | Hs.22982 | KIAA0958 protein | 2.3 |
| 104827 | AW052006 | Hs.8551 | PRP4/STK/WD splicing factor | 10.9 |
| 104846 | AI250789 | Hs.32478 | ESTs | 5.6 |
| 104854 | AA041276 | Hs.154729 | 3-phosphoinositide dependent protein kinase-1 | 12.3 |
| 104867 | AA278898 | Hs.225979 | hypothetical protein similar to small G proteins, especially RAP-2A | 2.0 |
| 104871 | T78044 | Hs.28893 | Homo sapiens mRNA; cDNA DKFZp564O2364 (from clone DKFZp564O2364) | 1.3 |
| 104896 | AW015318 | Hs.23165 | ESTs | 17.7 |
| 104909 | AW408164 | Hs.249184 | transcription factor 19 (SC1) | 5.0 |
| 104916 | AW958157 | Hs.155489 | NS1-associated protein 1 | 1.7 |
| 104919 | AA026880 | Hs.25252 | prolactin receptor | 1.4 |
| 104930 | AF043467 | Hs.32893 | neurexophilin 2 | 2.2 |
| 104973 | NM_015310 | Hs.6763 | KIAA0942 protein | 5.0 |
| 104974 | Y12059 | Hs.278675 | bromodomain-containing 4 | 1.4 |
| 104975 | AL136877 | Hs.50758 | SMC4 (structural maintenance of chromosomes 4, yeast)-like 1 | 2.4 |
| 104975 | AL136877 | Hs.50758 | SMC4 (structural maintenance of chromoso | 2.3 |
| 104978 | AI199268 | Hs.19322 | Homo sapiens, Similar to RIKEN cDNA 2010317E24 gene, clone IMAGE: 3502019, mRNA, partial cds | 7.2 |
| 104979 | AA937934 | Hs.321062 | ESTs | 1.3 |
| 104994 | AI499930 | Hs.334885 | mitochondrial GTP binding protein | 3.5 |
| 105009 | BE379584 | Hs.34789 | dolichyl-diphosphooligosaccharide-protein glycosyltransferase | 5.5 |
| 105012 | AF098158 | Hs.9329 | chromosome 20 open reading frame 1 | 3.3 |
| 105028 | AI050715 | Hs.2331 | E2F transcription factor 5, p130-binding | 2.2 |
| 105032 | AA127818 | | gb: zl12a02.s1 Soares_pregnant_uterus_NbHPU Homo sapiens cDNA clone IMAGE: 501674 3′ | 6.8 |
| 105039 | AA907305 | Hs.36475 | ESTs | 2.5 |
| 105041 | AB037716 | Hs.26204 | KIAA1295 protein | 2.2 |
| 105045 | BE242899 | Hs.129951 | speckle-type POZ protein | 3.8 |
| 105079 | AA151342 | Hs.12677 | CGI-147 protein | 9.5 |
| 105087 | AA147884 | Hs.9812 | Homo sapiens cDNA FLJ14388 fis, clone HEMBA1002716 | 5.6 |
| 105088 | H58589 | Hs.35156 | Homo sapiens cDNA FLJ11027 fis, clone PLACE1004114 | 2.2 |
| 105095 | Z78407 | Hs.27023 | vesicle transport-related protein | 2.2 |
| 105110 | BE387350 | Hs.33122 | KIAA1160 protein | 1.6 |
| 105126 | AW975433 | Hs.36288 | ESTs | 6.3 |
| 105127 | AA045648 | Hs.301957 | nudix (nucleoside diphosphate linked moiety X)-type motif 5 | 2.1 |
| 105141 | AA164687 | Hs.177576 | mannosyl (alpha-1,3-)-glycoprotein beta-1,4-N-acetylglucosaminyltransferase, isoenzyme A | 2.7 |
| 105158 | AW976357 | Hs.234545 | hypothetical protein NUF2R | 1.9 |
| 105169 | BE245294 | Hs.180789 | S164 protein | 1.7 |
| 105186 | AA191512 | Hs.28005 | Home sapiens cDNA FLJ11309 fis, clone PLACE1010076 | 4.8 |
| 105254 | AA071276 | Hs.19469 | KIAA0859 protein | 1.9 |
| 105281 | AA263143 | Hs.24596 | RAD51-interacting protein | 2.8 |
| 105288 | N99673 | Hs.3585 | ESTs, Weakly similar to AF126743 1 DNAJ domain-containing protein MCJ [H. sapiens] | 1.9 |
| 105302 | AA700122 | Hs.3355 | sentrin-specific protease | 8.0 |
| 105331 | AW270037 | Hs.179507 | KIAA0779 protein | 1.8 |
| 105359 | NM_016015 | Hs.8054 | CGI-68 protein | 8.2 |
| 105366 | BE264645 | Hs.282093 | hypothetical protein FLJ21918 | 5.0 |
| 105373 | AW887701 | Hs.32356 | hypothetical protein FLJ20628 | 2.5 |
| 105374 | BE242803 | Hs.262823 | hypothetical protein FLJ10326 | 2.2 |
| 105387 | AW592146 | Hs.108636 | membrane protein CH1 | 2.3 |
| 105393 | AF167570 | Hs.256583 | interleukin enhancer binding factor 3, 90 kD | 5.4 |
| 105399 | BE386877 | Hs.334811 | Npw38-binding protein NpwBP | 1.6 |
| 105400 | AF198620 | Hs.65648 | RNA binding motif protein 8A | 1.6 |
| 105445 | AA252395 | | gb: zs12g10.s1 NCI_CGAP_GCB1 Homo sapiens cDNA clone IMAGE: 685026 3′, mRNA sequence. | 5.0 |
| 105507 | BE268348 | Hs.226318 | CCR4-NOT transcription complex, subunit 7 | 1.6 |
| 105529 | AA113449 | Hs.32471 | hypothetical protein FLJ20364 | 1.3 |
| 105530 | AB023179 | Hs.9059 | KIAA0962 protein | 3.4 |
| 105547 | AA262640 | Hs.27445 | unknown | 9.3 |
| 105564 | BE616694 | Hs.288042 | hypothetical protein FLJ14299 | 1.4 |
| 105596 | AA579535 | Hs.18490 | hypothetical protein FLJ20452 | 10.9 |
| 105597 | AF054284 | Hs.334826 | splicing factor 3b, subunit 1, 155 kD | 2.9 |
| 105608 | AI808201 | Hs.287863 | hypothetical protein FLJ12475 | 1.7 |
| 105610 | AA280072 | Hs.99872 | fetal Alzheimer antigen | 1.4 |
| 105617 | AK000892 | Hs.4069 | glucocorticoid modulatory element binding protein 1 | 1.7 |
| 105620 | AW302245 | Hs.181390 | casein kinase 1, gamma 2 | 5.5 |
| 105658 | AA985190 | Hs.246875 | hypothetical protein FLJ20059 | 9.4 |
| 105697 | AW499988 | Hs.27801 | zinc finger protein 278 | 2.0 |
| 105708 | R26944 | Hs.180777 | Homo sapiens mRNA; cDNA DKFZp564M0264 (from clone DKFZp564M0264) | 1.7 |
| 105743 | BE246502 | Hs.9598 | sema domain, immunoglobulin domain (Ig), transmembrane domain (TM) and short | 2.6 |
| 105746 | AW151952 | Hs.46679 | hypothetical protein FLJ20739 | 1.5 |
| 105759 | AI123118 | Hs.15159 | chemokine-like factor, alternatively spliced | 1.3 |
| 105771 | AI267720 | Hs.153221 | synovial sarcoma, translocated to X chromosome | 1.6 |
| 105820 | AA741336 | Hs.152108 | transcriptional unit N143 | 2.2 |
| 105826 | AA478756 | Hs.194477 | E3 ubiquitin ligase SMURF2 | 1.3 |
| 105856 | AI262106 | Hs.12653 | ESTs | 2.4 |
| 105858 | AF151066 | Hs.281428 | hypothetical protein | 2.9 |
| 105875 | AK001708 | Hs.32271 | hypothetical protein FLJ10846 | 1.4 |
| 105930 | AF016371 | Hs.9880 | peptidyl prolyl isomerase H (cyclophilin H) | 5.2 |
| 106000 | AW194426 | Hs.20726 | ESTs | 1.7 |
| 106011 | AW081202 | Hs.12284 | Homo sapiens, clone IMAGE: 2989556, mRNA, partial cds | 2.8 |
| 106017 | AA477956 | Hs.26268 | ESTs | 1.4 |
| 106073 | AL157441 | Hs.17834 | downstream neighbor of SON | 1.4 |
| 106078 | AA130158 | Hs.19977 | ESTs, Moderately similar to ALU8_HUMAN ALU SUBFAMILY SX SEQUENCE CONTAMINATION | 1.6 |
| 106094 | AA533491 | Hs.23317 | hypothetical protein FLJ14681 | 6.8 |
| 106140 | AB006624 | Hs.14912 | KIAA0286 protein | 1.6 |
| 106271 | AA251393 | Hs.289052 | Homo sapiens, Similar to RIKEN cDNA 5430429M05 gene, clone MGC: 13155, mRNA, complete cds | 10.8 |
| 106288 | AB037742 | Hs.24336 | KIAA1321 protein | 1.3 |
| 106300 | Y10043 | Hs.19114 | high-mobility group (nonhistone chromosomal) protein 4 | 3.6 |
| 106333 | AL043114 | Hs.22410 | ESTs, Weakly similar to A54849 collagen alpha 1(VII) chain precursor [H. sapiens] | 5.4 |
| 106350 | AK001404 | Hs.194698 | cyclin B2 | 5.7 |
| 106359 | AW390282 | Hs.31130 | transmembrane 7 superfamily member 2 | 6.3 |
| 106381 | AB040916 | Hs.24106 | KIAA1483 protein | 6.5 |
| 106389 | AW748420 | Hs.6236 | Homo sapiens cDNA: FLJ21487 fis, clone COL05419 | 2.2 |
| 106457 | AF119256 | Hs.27801 | zinc finger protein 278 | 2.7 |
| 106470 | D63078 | Hs.186180 | Homo sapiens cDNA: FLJ23038 fis, clone LNG02039 | 2.3 |
| 106531 | AA454036 | Hs.8832 | ESTs | 1.6 |
| 106586 | AA243837 | Hs.57787 | ESTs | 1.6 |
| 106589 | AK000933 | Hs.28661 | Homo sapiens cDNA FLJ10071 fis, clone HEMBA1001702 | 2.4 |
| 106610 | AA458882 | Hs.79732 | fibulin 1 | 7.9 |
| 106624 | NM_003595 | Hs.26350 | tyrosylprotein sulfotransferase 2 | 7.7 |
| 106650 | AL049951 | Hs.22370 | Homo sapiens mRNA; cDNA DKFZp564O0122 (from clone DKFZp564O0122) | 1.8 |
| 106669 | AV657117 | Hs.184164 | ESTs, Moderately similar to S65657 alpha-1C-adrenergic receptor splice form 2 [H. sapiens] | 1.3 |
| 106713 | BE614802 | Hs.184352 | hypothetical protein FLJ12549 | 4.5 |
| 106717 | AA600357 | Hs.239489 | TIA1 cytotoxic granule-associated RNA-binding protein | 1.3 |
| 106723 | BE388094 | Hs.21857 | ESTs | 1.6 |
| 106795 | AF174487 | Hs.293753 | Bcl-2-related ovarian killer protein-like | 5.7 |
| 106829 | AW959893 | Hs.27099 | hypothetical protein FLJ23293 similar to ARL-6 interacting protein-2 | 16.2 |
| 106831 | BE564871 | Hs.29463 | centrin, EF-hand protein, 3 (CDC31 yeast homolog) | 1.5 |
| 106846 | AB037744 | Hs.34892 | KIAA1323 protein | 2.2 |
| 106852 | AF151031 | Hs.300631 | hypothetical protein | 1.3 |
| 106873 | N49809 | Hs.11197 | Homo sapiens, clone IMAGE: 3343149, mRNA, partial cds | 16.8 |
| 106886 | W79171 | Hs.9567 | GL002 protein | 1.5 |
| 106906 | AA861271 | Hs.222024 | transcription factor BMAL2 | 2.2 |
| 106920 | AK001838 | Hs.296323 | serum/glucocorticoid regulated kinase | 3.3 |
| 106945 | AK000511 | Hs.6294 | hypothetical protein DKFZp434L1435 similar to valyl tRNA synthetase | 6.8 |
| 106973 | BE156256 | Hs.11923 | hypothetical protein | 6.6 |
| 106977 | AL043152 | Hs.50421 | KIAA0203 gene product | 4.8 |
| 106978 | AW631480 | Hs.8688 | ESTs | 6.0 |
| 107004 | AA146872 | Hs.300700 | hypothetical protein FLJ20727 | 1.3 |
| 107029 | AF264750 | Hs.288971 | myeloid/lymphoid or mixed-lineage leukemia 3 | 1.8 |
| 107071 | AW385224 | Hs.35198 | ectonucleotide pyrophosphatase/phosphodiesterase 5 (putative function) | 1.7 |
| 107113 | AK000733 | Hs.23900 | GTPase activating protein | 2.5 |
| 107125 | AK000512 | Hs.69388 | hypothetical protein FLJ20505 | 1.7 |
| 107136 | AV661958 | Hs.8207 | GK001 protein | 4.6 |
| 107136 | AV661958 | Hs.8207 | GK001 protein | 3.3 |
| 107146 | AK001455 | Hs.5198 | Down syndrome critical region gene 2 | 2.0 |
| 107151 | AW378065 | Hs.8687 | ESTs | 6.3 |
| 107155 | AW391927 | Hs.7946 | KIAA1288 protein | 33.5 |
| 107174 | BE122762 | Hs.25338 | ESTs | 5.2 |
| 107197 | W15477 | Hs.64639 | glioma pathogenesis-related protein | 6.1 |
| 107221 | AW888411 | Hs.81915 | leukemia-associated phosphoprotein p18 (stathmin) | 17.4 |
| 107243 | BE219716 | Hs.34727 | ESTs, Moderately similar to I38759 zinc finger/leucine zipper protein [H. sapiens] | 7.4 |
| 107248 | AW263124 | Hs.315111 | nuclear receptor co-repressor/HDAC3 complex subunit | 1.8 |
| 107263 | D60341 | Hs.21198 | translocase of outer mitochondrial membrane 70 (yeast) homolog A | 6.6 |
| 107265 | BE379594 | Hs.49136 | ESTs, Moderately similar to ALU7_HUMAN ALU SUBFAMILY SQ SEQUENCE CONTAMINATION | 2.5 |
| 107298 | N95657 | Hs.6820 | ESTs, Moderately similar to YOJ1_CAEEL HYPOTHETICAL 63.5 KD PROTEIN ZK353.1 IN | 2.5 |
| 107298 | N95657 | Hs.6820 | ESTs, Moderately similar to YOJ1_CAEEL H | 1.7 |
| 107299 | BE277457 | Hs.30661 | hypothetical protein MGC4606 | 3.2 |
| 107316 | T63174 | Hs.193700 | Homo sapiens mRNA; cDNA DKFZp586I0324 (from clone DKFZp586I0324) | 2.0 |
| 107354 | NM_006299 | Hs.96448 | zinc finger protein 193 | 5.0 |
| 107392 | AW299900 | Hs.267632 | TATA element modulatory factor 1 | 1.2 |
| 107481 | AA307703 | Hs.279766 | kinesin family member 4A | 1.6 |
| 107529 | BE515065 | Hs.296585 | nucleolar protein (KKE/D repeat) | 3.0 |
| 107554 | AA001386 | Hs.59844 | ESTs | 1.3 |
| 107681 | BE379594 | Hs.49136 | ESTs, Moderately similar to ALU7_HUMAN ALU SUBFAMILY SQ SEQUENCE CONTAMINATION | 2.2 |
| 107772 | AA018587 | Hs.303055 | ESTs, Weakly similar to ALU1_HUMAN ALU SUBFAMILY J SEQUENCE CONTAMINATION | 2.1 |
| 107859 | AW732573 | Hs.47584 | potassium volstage-gated channel, delayed-rectifier, subfamily S, member 3 | 8.4 |
| 107901 | L42612 | Hs.335952 | keratin 6B | 2.5 |
| 107901 | L42612 | Hs.335952 | keratin 6B | 1.6 |
| 107922 | BE153855 | Hs.61460 | Ig superfamily receptor LNIR | 2.2 |
| 107974 | AW956103 | Hs.61712 | pyruvate dehydrogenase kinase, isoenzyme 1 | 6.7 |
| 108040 | AL121031 | Hs.159971 | SWI/SNF related, matrix associated, actin dependent regulator of chromatin, subfamily b, member 1 | 1.5 |
| 108230 | AA054224 | Hs.59847 | ESTs | 1.3 |
| 108274 | AF129535 | Hs.272027 | F-box only protein 5 | 7.1 |
| 108296 | N31256 | Hs.161623 | ESTs | 2.5 |
| 108496 | AA083069 | Hs.339659 | ESTs | 3.5 |
| 108607 | BE300380 | Hs.69476 | Homo sapiens cDNA FLJ12758 fis, clone NT2RP2001328 | 3.4 |
| 108621 | AA101809 | Hs.182685 | ESTs | 1.6 |
| 108634 | AW022410 | Hs.69507 | ESTs | 1.7 |
| 108647 | BE546947 | Hs.44276 | homeo box C10 | 9.8 |
| 108695 | AB029000 | Hs.70823 | KIAA1077 protein | 7.2 |
| 108717 | AA122393 | Hs.70811 | hypothetical protein FLJ20516 | 1.3 |
| 108740 | AI089575 | Hs.9071 | progesterone membrane binding protein | 2.7 |
| 108828 | AK001693 | Hs.273344 | DKFZP564O0463 protein | 1.8 |
| 108859 | AL121500 | Hs.178904 | ESTs | 1.5 |
| 108872 | H06720 | Hs.111680 | endosulfine alpha | 2.1 |
| 108891 | AI801235 | Hs.48480 | ESTs | 5.3 |
| 108894 | AK001431 | Hs.5105 | hypothetical protein FLJ10569 | 4.0 |
| 108955 | AA149754 | Hs.195155 | Homo sapiens amino acid transport system N2 (SN2) mRNA, complete cds | 5.6 |
| 108982 | AA151708 | Hs.171980 | homeo box (expressed in ES cells) 1 | 1.6 |
| 108987 | AA152178 | Hs.23467 | hypothetical protein FLJ10633 | 6.2 |
| 109002 | AB028987 | Hs.72134 | KIAA1064 protein | 1.7 |
| 109011 | AA156542 | Hs.72127 | ESTs | 1.4 |
| 109026 | AA157811 | | gb: zo35d07.s1 Stratagene colon (937204) Homo sapiens cDNA clone 3′ similar to contains Alu repetitive | 5.3 |
| 109068 | AA164293 | Hs.72545 | ESTs | 2.9 |
| 109101 | AW608930 | Hs.52184 | hypothetical protein FLJ20618 | 1.6 |
| 109112 | AW419196 | Hs.257924 | hypothetical protein FLJ13782 | 3.2 |
| 109124 | AK000684 | Hs.183887 | hypothetical protein FLJ22104 | 1.7 |
| 109139 | AJ132592 | Hs.59757 | zinc finger protein 281 | 2.6 |
| 109166 | AA219691 | Hs.73625 | RAB6 interacting, kinesin-like (rabkinesin 6) | 2.9 |
| 109198 | BE566742 | Hs.58169 | highly expressed in cancer, rich in leucine heptad repeats | 2.0 |
| 109213 | NM_016603 | Hs.82035 | potential nuclear protein C5ORF5; GAP-like protein | 5.3 |
| 109220 | AW958181 | Hs.189998 | ESTs | 5.7 |
| 109233 | AU077281 | Hs.170285 | nucleoporin 214 kD (CAIN) | 5.3 |
| 109270 | N99673 | Hs.3585 | ESTs, Weakly similar to AF126743 1 DNAJ domain-containing protein MCJ [H. sapiens] | 1.4 |
| 109273 | AA375752 | Hs.82719 | Homo sapiens mRNA; cDNA DKFZp586F1822 (from clone DKFZp586F1822) | 2.9 |
| 109313 | AF153201 | Hs.86276 | C2H2 (Kruppel-type) zinc finger protein | 1.3 |
| 109341 | AA213506 | Hs.115099 | EST | 2.9 |
| 109391 | AL096858 | Hs.184245 | KIAA0929 protein Msx2 interacting nuclear target (MINT) homolog | 1.5 |
| 109420 | H83603 | Hs.40408 | homeo box C9 | 2.2 |
| 109426 | N30531 | Hs.42215 | protein phosphatase 1, regulatory subunit 6 | 3.0 |
| 109429 | AI160029 | Hs.61438 | ESTs | 1.9 |
| 109445 | AA232103 | Hs.189915 | ESTs | 1.8 |
| 109450 | AB032969 | Hs.173042 | KIAA1143 protein | 3.7 |
| 109468 | NM_015310 | Hs.6763 | KIAA0942 protein | 3.2 |
| 109478 | AW074143 | Hs.87134 | ESTs | 2.0 |
| 109570 | L40027 | Hs.118890 | glycogen synthase kinase 3 alpha | 2.1 |
| 109662 | F02614 | Hs.27319 | ESTs | 1.4 |
| 109825 | R71264 | Hs.16798 | ESTs | 1.3 |
| 110039 | H11938 | Hs.21907 | histone acetyltransferase | 2.0 |
| 110056 | AA503041 | Hs.279009 | matrix Gla protein | 2.5 |
| 110085 | AA603840 | Hs.29956 | KIAA0460 protein | 1.7 |
| 110110 | T07353 | Hs.7948 | ESTs | 2.9 |
| 110129 | R51853 | Hs.226429 | ESTs, Weakly similar to ALU1_HUMAN ALU SUBFAMILY J SEQUENCE CONTAMINATION | 1.7 |
| 110154 | NM_014521 | Hs.17667 | SH3-domain binding protein 4 | 4.2 |
| 110240 | AI668594 | Hs.176588 | ESTs, Weakly similar to CP4Y_HUMAN CYTOCHROME P450 4A11 PRECURSOR [H. sapiens] | 4.2 |
| 110242 | N41744 | Hs.19978 | CGI-30 protein | 1.3 |
| 110259 | H28428 | Hs.32406 | ESTs, Weakly similar to I38022 hypothetical protein [H. sapiens] | 2.2 |
| 110312 | BE256986 | Hs.11896 | hypothetical protein FLJ12089 | 2.1 |
| 110330 | AI288666 | Hs.16621 | DKFZP434I116 protein | 6.2 |
| 110501 | H55748 | | gb: yq94a01.s1 Soares fetal liver spleen 1NFLS Homo sapiens cDNA clone IMAGE: 203400 3′ | 6.1 |
| 110504 | H55915 | Hs.210859 | hypothetical protein FLJ11016 | 6.1 |
| 110525 | H57330 | Hs.37430 | EST | 6.3 |
| 110568 | AK001160 | Hs.5999 | hypothetical protein FLJ10298 | 1.3 |
| 110699 | T97586 | Hs.18090 | ESTs | 1.8 |
| 110705 | AB007902 | Hs.32168 | KIAA0442 protein | 1.6 |
| 110742 | AW190338 | Hs.28029 | hypothetical protein MGC11256 | 7.6 |
| 110761 | AL138077 | Hs.16157 | hypothetical protein FLJ12707 | 2.5 |
| 110762 | BE044245 | Hs.30011 | hypothetical protein MGC2963 | 9.3 |
| 110765 | AK000322 | Hs.18457 | hypothetical protein FLJ20315 | 5.5 |
| 110769 | BE000831 | Hs.23837 | Homo sapiens cDNA FLJ11812 fis, clone HEMBA1006364 | 2.1 |
| 110799 | AI089660 | Hs.323401 | dpy-30-like protein | 1.5 |
| 110805 | T25829 | Hs.24048 | FK506 binding protein precursor | 6.6 |
| 110813 | AA767373 | Hs.35669 | ESTs, Moderately similar to ALU1_HUMAN ALU SUBFAMILY J SEQUENCE CONTAMINATION | 5.7 |
| 110820 | R33261 | Hs.6614 | ESTs, Weakly similar to A43932 mucin 2 precursor, intestinal [H. sapiens] | 3.4 |
| 110840 | N31598 | Hs.12727 | hypothetical protein FLJ21610 | 1.7 |
| 110844 | AI740792 | Hs.167531 | methylcrotonoyl-Coenzyme A carboxylase 2 (beta) | 1.7 |
| 110854 | BE612992 | Hs.27931 | hypothetical protein FLJ10607 similar to glucosamine-phosphate N-acetyltransferase | 4.7 |
| 110856 | AA992380 | | gb: ot37g06.s1 Soares_testis_NHT Homo sapiens cDNA clone 3′ similar to contains element | 2.3 |
| 110885 | BE384447 | Hs.16034 | hypothetical protein MGC13186 | 3.5 |
| 110897 | AL117430 | Hs.6880 | DKFZP434D156 protein | 2.2 |
| 110915 | BE092285 | Hs.29724 | hypothetical protein FLJ13187 | 2.6 |
| 110918 | H04360 | Hs.24283 | ESTs, Moderately similar to reduced expression in cancer [H. sapiens] | 1.9 |
| 110958 | NM_005864 | Hs.24587 | signal transduction protein (SH3 containing) | 6.7 |
| 110963 | AK002180 | Hs.11449 | DKFZP564O123 protein | 2.0 |
| 110981 | AK001980 | Hs.24284 | ADP-ribosyltransferase (NAD+; poly(ADP-ribose) polymerase)-like 2 | 1.3 |
| 110984 | AW613287 | Hs.80120 | UDP-N-acetyl-alpha-D-galactosamine: polypeptide N-acetylgalactosaminyltransferase 1 (GaINAc-T1) | 1.8 |
| 111125 | N63823 | Hs.269115 | ESTs, Moderately similar to Z195_HUMAN ZINC FINGER PROTEIN 195 [H. sapiens] | 3.6 |
| 111132 | AB037807 | Hs.83293 | hypothetical protein | 2.1 |
| 111164 | N46180 | Hs.122489 | Homo sapiens cDNA FLJ13289 fis, clone OVARC1001170 | 2.3 |
| 111172 | R67419 | Hs.21851 | Homo sapiens cDNA FLJ12900 fis, clone NT2RP2004321 | 3.7 |
| 111174 | AL050166 | Hs.26295 | Homo sapiens mRNA; cDNA DKFZp586D1122 (from clone DKFZp586D1122) | 7.5 |
| 111179 | AK000136 | Hs.10760 | asporin (LRR class 1) | 7.1 |
| 111184 | AI815486 | Hs.243901 | Homo sapiens cDNA FLJ20738 fis, clone HEP08257 | 6.7 |
| 111184 | AI815486 | Hs.243901 | Homo sapiens cDNA FLJ20738 fis, clone HE | 3.3 |
| 111189 | N67603 | Hs.272130 | ESTs, Weakly similar to S65824 reverse transcriptase homolog [H. sapiens] | 3.6 |
| 111216 | AW139408 | Hs.152940 | ESTs | 1.5 |
| 111221 | AB037782 | Hs.15119 | KIAA1361 protein | 2.6 |
| 111223 | AA852773 | Hs.334838 | KIAA1866 protein | 4.6 |
| 111239 | N90956 | Hs.17230 | hypothetical protein FLJ22087 | 7.9 |
| 111285 | AA778711 | Hs.4310 | eukatyotic translation initiation factor 1A | 6.9 |
| 111299 | AB033091 | Hs.74313 | KIAA1265 protein | 5.0 |
| 111312 | AI523913 | Hs.34504 | ESTs | 3.8 |
| 111318 | T99755 | Hs.334728 | ESTs | 1.2 |
| 111337 | AA837396 | Hs.263925 | LIS1-interacting protein NUDE1, rat homolog | 5.1 |
| 111352 | H58589 | Hs.35156 | Homo sapiens cDNA FLJ11027 fis, clone PLACE1004114 | 2.2 |
| 111370 | AI478658 | Hs.94631 | brefeldin A-inhibited guanine nucleotide-exchange protein 1 | 2.8 |
| 111384 | N94606 | Hs.288969 | HSCARG protein | 2.2 |
| 111389 | AK000987 | Hs.169111 | oxidation resistance 1 | 2.1 |
| 111391 | NM_003896 | Hs.225939 | sialyltransferase 9 (CMP-NeuAc: lactosylceramide alpha-2,3-sialyltransferase; GM3 synthase) | 5.1 |
| 111392 | W46342 | Hs.325081 | Homo sapiens, clone IMAGE: 3659680, mRNA, partial cds | 8.4 |
| 111452 | R02354 | Hs.15999 | ESTs | 2.7 |
| 111486 | AI051194 | Hs.227978 | EST | 6.5 |
| 111549 | W90638 | Hs.20321 | ESTs, Moderately similar to ZRF1_HUMAN ZUOTIN RELATED FACTOR-1 (M-PHASE | 1.4 |
| 111585 | R10720 | Hs.20670 | EST | 1.6 |
| 111627 | R52656 | Hs.21691 | ESTs | 1.6 |
| 111870 | AB037834 | Hs.18685 | Homo sapiens mRNA for KIAA1413 protein, partial cds | 2.4 |
| 111937 | BE298665 | Hs.14846 | Homo sapiens mRNA; cDNA DKFZp564D016 (from clone DKFZp564D016) | 10.6 |
| 111944 | AW083791 | Hs.21263 | suppressor of potassium transport defect 3 | 6.6 |
| 111987 | NM_015310 | Hs.6763 | KIAA0942 protein | 5.1 |
| 112134 | R41823 | Hs.7413 | ESTs; calsyntenin-2 | 2.8 |
| 112244 | AB029000 | Hs.70823 | KIAA1077 protein | 14.6 |
| 112388 | R46071 | Hs.301693 | Homo sapiens, clone IMAGE: 3638994, mRNA, partial cds | 9.0 |
| 112456 | NM_016248 | Hs.232076 | A kinase (PRKA) anchor protein 11 | 1.4 |
| 112464 | AW007287 | Hs.28538 | Homo sapiens cDNA: FLJ21086 fis, clone CAS03272 | 1.4 |
| 112506 | AI742756 | Hs.26079 | ESTs | 3.2 |
| 112513 | R68425 | Hs.13809 | hypothetical protein FLJ10648 | 2.0 |
| 112752 | AK001635 | Hs.14838 | hypothetical protein FLJ10773 | 1.8 |
| 112884 | AK000004 | Hs.5013 | Homo sapiens mRNA for FLJ00004 protein, partial cds | 6.6 |
| 112923 | T10258 | Hs.5037 | EST | 1.5 |
| 112936 | AW970826 | Hs.6185 | KIAA1557 protein | 3.2 |
| 112958 | R61388 | Hs.6724 | ESTs | 6.0 |
| 112966 | Z44718 | Hs.102548 | glucocorticoid receptor DNA binding factor 1 | 6.4 |
| 112978 | AK000272 | Hs.7099 | hypothetical protein FLJ20265 | 1.2 |
| 112995 | AA737033 | Hs.7155 | ESTs, Moderately similar to 2115357A TYKi protein [M. musculus] | 5.6 |
| 112996 | BE276112 | Hs.7165 | zinc finger protein 259 | 2.0 |
| 113047 | AI571940 | Hs.7549 | ESTs | 1.9 |
| 113049 | AW965190 | Hs.7560 | Homo sapiens mRNA for KIAA1729 protein, partial cds | 2.4 |
| 113089 | T40707 | Hs.270862 | ESTs | 1.3 |
| 113196 | T57317 | | gb: yb51a03.s1 Stratagene fetal spleen (937205) Homo sapiens cDNA clone IMAGE: 74668 3′, | 1.7 |
| 113248 | T63857 | | gb: yc16e01.s1 Stratagene lung (937210) Homo sapiens cDNA clone 3′, mRNA sequence | 2.8 |
| 113254 | AK002180 | Hs.11449 | DKFZP564O123 protein | 1.3 |
| 113277 | AW971049 | Hs.11774 | protein (peptidyl-prolyl cis/trans isomerase) NIMA-interacting, 4 (parvulin) | 3.2 |
| 113429 | AA688021 | Hs.179808 | ESTs | 1.2 |
| 113499 | AI467908 | Hs.8882 | ESTs | 5.9 |
| 113547 | H59588 | Hs.15233 | ESTs | 2.0 |
| 113554 | AW503990 | Hs.142442 | HP1-BP74 | 3.6 |
| 113647 | AA813887 | Hs.188173 | Homo sapiens cDNA FLJ12187 fis, clone MAMMA1000831 | 1.3 |
| 113702 | T97307 | | gb: ye53h05.s1 Soares fetal liver spleen 1NFLS Homo sapiens cDNA clone IMAGE: 121497 3′, | 4.4 |
| 113722 | AV653556 | Hs.184411 | albumin | 1.3 |
| 113759 | AW499665 | Hs.9456 | SWI/SNF related, matrix associated, actin dependent regulator of chromatin, subfamily a, member 5 | 1.2 |
| 113777 | BE266947 | Hs.10590 | zinc finger protein313 | 13.4 |
| 113783 | AL359588 | Hs.7041 | hypothetical protein DKFZp762B226 | 1.7 |
| 113791 | AI269096 | Hs.135578 | chitobiase, di-N-acetyl- | 1.3 |
| 113808 | W44735 | Hs.9286 | Homo sapiens cDNA: FLJ21278 fis, clone COL01832 | 3.3 |
| 113811 | BE207480 | Hs.6994 | Homo sapiens cDNA: FLJ22044 fis, clone HEP09141 | 3.1 |
| 113817 | H13325 | Hs.332795 | hypothetical protein DKFZp761O17121 | 3.2 |
| 113826 | AW378212 | Hs.24809 | hypothetical protein FLJ10826 | 2.3 |
| 113834 | T26483 | Hs.6059 | EGF-containing fibulin-like extracellular matrix protein 2 | 11.3 |
| 113868 | W57902 | Hs.90744 | proteasome (prosome, macropain) 26S subunit, non-ATPase, 11 | 2.7 |
| 113870 | AL079314 | Hs.16537 | hypothetical protein, similar to (U06944) PRAJA1 | 6.1 |
| 113885 | AW959486 | Hs.21732 | ESTs | 6.6 |
| 113923 | AW953484 | Hs.3849 | hypothetical protein FLJ22041 similar to FK506 binding proteins | 1.9 |
| 113989 | W87544 | Hs.268828 | ESTs | 1.2 |
| 114022 | AI539519 | Hs.120969 | Homo sapiens cDNA FLJ11562 fis, clone HEMBA1003197 | 5.4 |
| 114030 | AI825386 | Hs.164478 | hypothetical protein FLJ21939 similar to 5-azacytidine induced gene 2 | 9.4 |
| 114060 | AB029551 | Hs.7910 | RING1 and YY1 binding protein | 1.8 |
| 114196 | AF017445 | Hs.150926 | fucose-1-phosphate guanylyltransferase | 1.5 |
| 114226 | AB028968 | Hs.7989 | KIAA1045 protein | 1.8 |
| 114253 | BE149866 | Hs.14831 | Homo sapiens, Similar to zinc finger protein 136 (clone pHZ-20), | 2.3 |
| | | | clone MGC: 10647, mRNA, complete cds | |
| 114262 | AL117518 | Hs.3686 | KIAA0978 protein | 1.4 |
| 114275 | AW515443 | Hs.306117 | KIAA0306 protein | 15.8 |
| 114292 | AI815395 | Hs.184641 | fatty acid desaturase 2 | 1.9 |
| 114309 | AA332453 | Hs.20824 | CGI-85 protein | 2.4 |
| 114392 | AA249590 | Hs.100748 | ESTs, Weekly similar to A28996 proline-rich protein M14 precursor - mouse [M. musculus] | 1.8 |
| 114407 | BE539976 | Hs.103305 | Homo sapiens mRNA; cDNA DKFZp434B0425 (from clone DKFZp434B0425) | 1.2 |
| 114455 | H37908 | Hs.271616 | ESTs, Weakly similar to ALU8_HUMAN ALU SUBFAMILY SX SEQUENOE | 5.5 |
| 114463 | AL120247 | Hs.40109 | KIAA0872 protein | 5.2 |
| 114464 | AI091713 | Hs.106597 | Homo sapiens, Similar to RIKEN cDNA 1110012M11 gene, clone IMAGE: 3688605, mRNA, partial cds | 1.2 |
| 114471 | AA028074 | Hs.104613 | RP42 homolog | 1.8 |
| 114400 | BE066778 | Hs.151678 | UDP-N-acetyl-alpha-D-galactosamine: polypeptide N-acetylgalactosaminyltransferase 6 (GalNAc-T6) | 13.4 |
| 114671 | AA766268 | Hs.266273 | hypothetical protein FLJ13346 | 1.9 |
| 114698 | AA476966 | Hs.110857 | polymerase (RNA) III (DNA directed) polypeptide K (12.3 kDa) | 3.5 |
| 114730 | AI373544 | Hs.331328 | intermediate filament protein syncoilin | 3.8 |
| 114767 | AI859865 | Hs.154443 | minichromosome maintenance deficient (S. cerevisiae) 4 | 1.6 |
| 114774 | AV656017 | Hs.184325 | CGI-76 protein | 3.1 |
| 114798 | AA159181 | Hs.54900 | serologically defined colon cancer antigen 1 | 3.5 |
| 114860 | AL157545 | Hs.42179 | bromodomain and PHD finger containing, 3 | 4.3 |
| 114895 | AA236177 | Hs.76591 | KIAA0887 protein | 7.1 |
| 114896 | BE539101 | Hs.5324 | hypothetical protein | 1.3 |
| 114911 | AA236672 | | gb: zt29f02.s1 Soares ovary tumor NbHOT Homo sapiens cDNA clone IMAGE: | 1.5 |
| | | | 723771 3′, mRNA sequence. | |
| 114930 | AA237022 | Hs.188717 | ESTs | 2.0 |
| 114938 | AA242834 | Hs.58364 | ESTs | 2.9 |
| 114965 | AI733881 | Hs.72472 | BMP-R1B | 2.3 |
| 115023 | AF102546 | Hs.63931 | dachshund (Drosophila) homolog | 1.3 |
| 115038 | AA252360 | Hs.87968 | toll-like receptor 9 | 1.6 |
| 115061 | AI751438 | Hs.41271 | Homo sapiens mRNA full length insert cDNA clone EUROIMAGE 1913076 | 11.8 |
| 115062 | AA253314 | Hs.154103 | LIM protein (similar to rat protein kina | 1.5 |
| 115117 | AI670847 | Hs.5324 | hypothetical protein | 1.5 |
| 115121 | AI634549 | Hs.88155 | ESTs | 2.8 |
| 115206 | AW183695 | Hs.186572 | ESTs | 2.5 |
| 115221 | AW365434 | Hs.79741 | hypothetical protein FLJ10116 | 1.5 |
| 115239 | BE251328 | Hs.73291 | hypothetical protein FLJ10881 | 1.3 |
| 115242 | AI368236 | Hs.283732 | ESTs, Moderately similar to ALU1_HUMAN ALU SUBFAMILY J SEQUENCE | 1.4 |
| 115278 | AK002163 | Hs.301724 | hypothetical protein FLJ11301 | 1.5 |
| 115285 | AW972872 | Hs.293736 | ESTs | 2.4 |
| 115291 | BE545072 | Hs.122579 | hypothetical protein FLJ10461 | 6.2 |
| 115400 | AI215069 | Hs.89113 | ESTs | 6.6 |
| 115468 | AA314349 | Hs.48499 | tumor antigen SLP-8p | 7.4 |
| 115471 | AK001376 | Hs.59346 | hypothetical protein FLJ10514 | 1.4 |
| 115479 | AW301608 | Hs.278188 | ESTs, Moderately similar to I54374 gene NF2 protein [H. sapiens] | 4.0 |
| 115496 | AW247593 | Hs.71819 | eukaryotic translation initiation factor 4E binding protein 1 | 16.3 |
| 115500 | Y14443 | Hs.88219 | zinc finger protein 200 | 5.0 |
| 115553 | AJ275986 | Hs.71414 | transcription factor (SMIF gene) | 2.5 |
| 115581 | AI540842 | Hs.61082 | ESTs | 6.1 |
| 115587 | BE081342 | Hs.283037 | HSPC039 protein | 2.9 |
| 115590 | AA399477 | Hs.67896 | 7-60 protein | 5.3 |
| 115646 | N36110 | Hs.305971 | solute carrier family 2 (facilitated glucose transporter), member 10 | 4.7 |
| 115652 | BE093589 | Hs.38178 | hypothetical protein FLJ23468 | 10.6 |
| 115655 | AL048269 | Hs.288544 | Homo sapiens, clone MGC: 16063, mRNA, complete cds | 12.7 |
| 115663 | AI138785 | Hs.40507 | ESTs | 2.0 |
| 115676 | AA953006 | Hs.88143 | ESTs | 3.0 |
| 115690 | AA625132 | Hs.44159 | hypothetical protein FLJ21615 | 1.7 |
| 115693 | AF231023 | Hs.55173 | cadherin, EGF LAG seven-pass G-type receptor 3, flamingo (Drosophila) homolog | 6.8 |
| 115715 | BE395161 | Hs.1390 | proteasome (prosome, macropain) subunit beta type, 2 | 1.7 |
| 115734 | AI950339 | Hs.40782 | ESTs | 2.6 |
| 115811 | NM_015434 | Hs.48604 | DKFZP434B168 protein | 2.1 |
| 115823 | AI732742 | Hs.87440 | ESTs | 2.1 |
| 115837 | AI675217 | Hs.42761 | ESTs | 1.3 |
| 115844 | AI373062 | Hs.332938 | hypothetical protein MGC5370 | 4.4 |
| 115866 | AW062629 | Hs.52081 | KIAA0867 protein | 7.2 |
| 115875 | N55669 | Hs.333823 | mitochondrial ribosomal protein L13 | 1.2 |
| 115941 | AI867451 | Hs.46679 | hypothetical protein FLJ20739 | 5.5 |
| 115968 | AB037753 | Hs.62767 | KIAA1332 protein | 9.8 |
| 116003 | BE275469 | Hs.66493 | Down syndrome critical region gene 5 | 1.4 |
| 116011 | AL359053 | Hs.57664 | Homo sapiens mRNA full length insert cDNA clone EUROIMAGE 2005735 | 2.4 |
| 116108 | AA770688 | Hs.28777 | H2A histone family, member L | 1.8 |
| 116134 | BE243834 | Hs.50441 | CGI-04 protein | 1.4 |
| 116189 | N35719 | Hs.44749 | ESTs, Moderately similar to T00358 hypothetical protein KIAA0684 [H. sapiens] | 1.2 |
| 116195 | AW821113 | Hs.72402 | ESTs | 2.1 |
| 116238 | AV660717 | Hs.47144 | DKFZP586N0819 protein | 1.7 |
| 116246 | AF265555 | Hs.250646 | baculoviral IAP repeat-containing 6 | 1.7 |
| 116262 | AI936442 | Hs.59838 | hypothetical protein FLJ10808 | 1.7 |
| 116298 | AI955411 | Hs.94109 | Homo sapiens cDNA FLJ13634 fis, clone PLACE1011133 | 1.9 |
| 116318 | AF097645 | Hs.58570 | deleted in cancer 1; RNA helicase HDB/DICE1 | 4.9 |
| 116325 | AI472106 | Hs.49303 | Homo sapiens cDNA FLJ11663 fis, clone HEMBA1004631 | 1.4 |
| 116336 | AL133033 | Hs.4084 | KIAA1025 protein | 1.9 |
| 116339 | AK000290 | Hs.44033 | dipeptidyl peptidase 8 | 1.5 |
| 116350 | AA497129 | Hs.184771 | nuclear factor I/C (CCAAT-binding transcription factor) | 1.9 |
| 116358 | AI149586 | Hs.38125 | interferon-induced protein 75, 52 kD | 1.9 |
| 116365 | N50174 | Hs.46765 | ESTs | 6.1 |
| 116368 | N90466 | Hs.71109 | KIAA1229 protein | 1.6 |
| 116417 | AW499664 | Hs.12484 | Human clone 23826 mRNA sequence | 7.4 |
| 116436 | AA161411 | Hs.58668 | chromosome 21 open reading frame 57 | 2.1 |
| 116462 | AF218313 | Hs.236828 | putative helicase RUVBL | 1.5 |
| 116470 | AI272141 | Hs.83484 | SRY (sex determining region Y)-box 4 | 2.1 |
| 116470 | AI272141 | Hs.83484 | SRY (sex determining region Y)-box 4 | 1.2 |
| 116575 | AA312572 | Hs.6241 | phosphoinositide-3-kinase, regulatory subunit, polypeptide 1 (p85 alpha) | 1.5 |
| 116637 | AK001043 | Hs.92033 | integrin-linked kinase-associated serine/threonine phosphatase 2C | 2.7 |
| 116640 | X89984 | Hs.211563 | B-cell CLL/lymphoma 7A | 2.3 |
| 116700 | AI800202 | Hs.317589 | hypothetical protein MGC10765 | 1.4 |
| 116705 | AW074819 | Hs.12313 | hypothetical protein FLJ14566 | 3.4 |
| 116732 | AW152225 | Hs.165909 | ESTs, Weakly similar to I38022 hypothetical protein [H. sapiens] | 2.9 |
| 116921 | AW068115 | Hs.821 | biglycan | 8.3 |
| 116926 | H73608 | Hs.290830 | ESTs | 1.7 |
| 117034 | U72209 | Hs.180324 | YY1-associated factor 2 | 3.4 |
| 117132 | AI393666 | Hs.42315 | p10-binding protein | 5.2 |
| 117247 | N21032 | | gb: yx46f06.s1 Soares melanocyte 2NbHM Homo sapiens cDNA clone IMAGE: | 5.5 |
| | | | 264803 3′, mRNA sequence. | |
| 117276 | N71183 | Hs.121806 | Homo sapiens cDNA FLJ11971 fis, clone HEMBB1001208 | 1.5 |
| 117284 | AK001701 | Hs.183779 | Homo sapiens cDNA FLJ10590 fis, clone NT2RP2004392, weakly similar to MNN4 PROTEIN | 2.0 |
| 117367 | AI041793 | Hs.42502 | ESTs | 2.0 |
| 117368 | AI878942 | Hs.90336 | ATPase, H + transporting, lysosomal (vacuolar proton pump), member J | 2.1 |
| 117382 | AF150275 | Hs.40173 | ESTs | 2.7 |
| 117412 | N32536 | Hs.42645 | solute carrierfamily 16 (monocarboxylic acid transporters), member 6 | 1.4 |
| 117557 | AF123050 | Hs.44532 | diubiquitin | 3.4 |
| 117588 | N34895 | Hs.44648 | ESTs | 3.4 |
| 117745 | BE294925 | Hs.46680 | CGI-12 protein | 3.0 |
| 117754 | AA121673 | Hs.59757 | zinc finger protein 281 | 1.9 |
| 117879 | N54706 | Hs.303025 | chromosome 11 open reading frame 24 | 1.8 |
| 117881 | AF161470 | Hs.260622 | butyrate-induced transcript 1 | 5.7 |
| 117904 | BE540675 | Hs.332938 | hypothetical protein MGC5370 | 5.9 |
| 117911 | AL137379 | Hs.47125 | hypothetical protein FLJ13912 | 1.7 |
| 117933 | Y10518 | Hs.116470 | hypothetical protein FLJ20048 | 1.7 |
| 117983 | AL110246 | Hs.47367 | KIAA1785 protein | 5.4 |
| 118078 | N54321 | Hs.47790 | EST | 5.2 |
| 118301 | AA453902 | Hs.293264 | ESTs | 2.6 |
| 118429 | AA243332 | Hs.74649 | cytochrome c oxidase subunit VIc | 2.5 |
| 118472 | AL157545 | Hs.42179 | bromodomain and PHD finger containing, 3 | 4.1 |
| 118488 | AJ277275 | Hs.50102 | rapa-2 (rapa gene) | 1.2 |
| 118509 | N22617 | Hs.43228 | Homo sapiens cDNA FLJ11835 fis, clone HEMBA1006595 | 1.5 |
| 118528 | AI949952 | Hs.49397 | ESTs | 7.4 |
| 118656 | AI458020 | Hs.293287 | ESTs | 2.5 |
| 118670 | AA332845 | Hs.152618 | ESTs, Moderately similar to ZN91_HUMAN ZINC FINGER PROTEIN 91 [H. sapiens] | 1.2 |
| 118698 | AB033113 | Hs.50187 | KIAA1287 protein | 2.1 |
| 118737 | AA199686 | | gb: zq75g09.r1 Stratagene hNT neuron (937233) Homo sapiens cDNA clone IMAGE: 647488 5′ | 5.2 |
| 118925 | N92293 | Hs.206832 | ESTs, Moderately similar to ALU8_HUMAN ALU SUBFAMILY SX SEQUENCE CONTAMINATION | 1.4 |
| 118984 | AI668709 | Hs.240722 | ESTs, Moderately similar to ALU8_HUMAN ALU SUBFAMILY SX SEQUENCE CONTAMINATION | 3.6 |
| 118986 | AF148713 | Hs.125830 | bladder cancer overexpressed protein | 4.8 |
| 119206 | W24781 | Hs.293798 | KIAA1710 protein | 1.7 |
| 119235 | AW453069 | Hs.3657 | activity-dependent neuroprotective protein | 2.2 |
| 119235 | AW453069 | Hs.3657 | activity-dependent neuroprotective prote | 1.6 |
| 119265 | BE539706 | Hs.285363 | ESTs | 1.4 |
| 119279 | N57568 | Hs.48028 | EST | 25.1 |
| 119298 | NM_001241 | Hs.155478 | cyclinT2 | 1.6 |
| 119338 | AI417240 | Hs.320836 | ESTs, Weakly similar to A47582 B-cell growth factor precursor [H. sapiens] | 1.3 |
| 119349 | T65004 | Hs.163561 | ESTs | 8.4 |
| 119403 | AL117554 | Hs.119908 | nucleolar protein NOP5/NOP58 | 6.7 |
| 119478 | AI624342 | Hs.170042 | ESTs | 2.4 |
| 119486 | AI796730 | Hs.55513 | ESTs | 2.1 |
| 119513 | W37933 | | Empirically selected from AFFX single probeset | 1.9 |
| 119601 | AK000155 | Hs.91684 | Homo sapiens mRNA; cDNA DKFZp667I103 (from clone DKFZp667I103) | 3.7 |
| 119602 | AW675298 | Hs.233694 | hypothetical protein FLJ11350 | 3.0 |
| 119676 | AA243837 | Hs.57787 | ESTs | 1.4 |
| 119682 | W61019 | Hs.57811 | ESTs | 1.2 |
| 119774 | AB032977 | Hs.6298 | KIAA1151 protein | 1.8 |
| 119780 | NM_016625 | Hs.191381 | hypothetical protein | 3.1 |
| 119789 | BE393948 | Hs.50915 | kallikrein 5 (KLK5; KLK-L2; stratum corneum tryptic enzyme) | 9.2 |
| 119805 | AJ223810 | Hs.43213 | ESTs, Weakly similar to IEFS_HUMAN TRANSFORMATION-SENSITIVE PROTEIN IEF SSP | 3.6 |
| 119818 | AA130970 | Hs.58382 | hypothetical protein FLJ11101 | 2.5 |
| 119863 | AA081218 | Hs.58608 | Homo sapiens cDNA FLJ14206 fis, clone NT2RP3003157 | 2.7 |
| 119905 | AW449064 | Hs.119571 | collagen, type III, alpha 1 (Ehlers-Danlos syndrome type IV, autosomal dominant) | 2.6 |
| 119966 | AA703129 | Hs.58963 | ESTs | 2.7 |
| 120132 | W57554 | Hs.125019 | lymphoid nuclear protein (LAF-4) mRNA | 1.2 |
| 120206 | H26735 | Hs.91668 | Home sapiens clone PP1498 unknown mRNA | 45.7 |
| 120248 | AI924294 | Hs.173259 | uncharacterized bone marrow protein BM033 | 1.2 |
| 120253 | AA131376 | Hs.326401 | fibroblast growth factor 12B | 38.9 |
| 120269 | AW131940 | Hs.104030 | ESTs | 9.6 |
| 120274 | AA177051 | | gb: nc02a02.s1 NCI_CGAP_Pr3 Homo sapiens cDNA clone IMAGE: 194 similar to contains Alu | 4.6 |
| 120280 | AA190577 | | gb: zp52g02.s1 Stratagene HeLa cell s3 937216 Homo sapiens cDNA clone 3′, mRNA sequence | 2.0 |
| 120296 | AW995911 | Hs.299883 | hypothetical protein FLJ23399 | 1.8 |
| 120297 | AA191384 | Hs.104072 | ESTs, Weakly similar to Z195_HUMAN ZINC FINGER PROTEIN 195 [H. sapiens] | 15.2 |
| 120324 | AA195517 | Hs.191643 | ESTs | 5.5 |
| 120325 | AA195651 | Hs.104106 | ESTs | 6.4 |
| 120327 | AK000292 | Hs.278732 | hypothetical protein FLJ20285 | 16.1 |
| 120336 | N85785 | Hs.181165 | eukaryotic translation elongation factor 1 alpha 1 | 2.9 |
| 120342 | AW450669 | Hs.45068 | hypothetical protein DKFZp434I143 | 5.7 |
| 120345 | AA210722 | Hs.104158 | ESTs | 4.5 |
| 120349 | AW969481 | Hs.55189 | hypothetical protein | 16.8 |
| 120352 | R06859 | Hs.193172 | ESTs, Weakly similar to I38022 hypothetical protein [H. sapiens] | 5.0 |
| 120356 | AF000545 | Hs.296433 | putative purinergic receptor | 28.1 |
| 120371 | AA219305 | Hs.104196 | EST | 12.4 |
| 120382 | AA228026 | Hs.38774 | ESTs | 4.0 |
| 120383 | AL109963 | Hs.123122 | FSH primary response (LRPR1, rat) homolog 1 | 9.7 |
| 120386 | AW969665 | Hs.154848 | hypothetical protein DKFZp434D0127 | 32.6 |
| 120388 | AA232874 | Hs.104245 | ESTs | 3.1 |
| 120389 | AW967985 | Hs.325572 | ESTs, Moderately similar to ALU7_HUMAN ALU SUBFAMILY SQ SEQUENCE CONTAMINATION | 21.7 |
| 120396 | AA134006 | Hs.79306 | eukaryotic translation initiation factor 4E | 12.5 |
| 120404 | AB023230 | Hs.96427 | KIAA1013 protein | 7.2 |
| 120418 | AW966893 | Hs.26613 | Homo sapiens mRNA; cDNA DKFZp586F1323 (from clone DKFZp586F1323) | 11.4 |
| 120423 | AA236453 | Hs.18978 | Homo sapiens cDNA: FLJ22822 fis, clone KAIA3968 | 1.9 |
| 120472 | AI950087 | | gb: wq05c02.x1 NCI_CGAP_Kid12 Homo sapiens cDNA clone 3′, mRNA sequence | 19.4 |
| 120473 | AA251973 | Hs.269988 | ESTs | 5.4 |
| 120484 | AA253170 | Hs.96473 | EST | 10.4 |
| 120504 | AA256837 | | gb: zr84d10.s1 Soares_NhHMPu_S1 Homo sapiens cDNA clone IMAGE: 682387 3′, mRNA sequence. | 3.9 |
| 120509 | BE047718 | Hs.96545 | ESTs | 9.4 |
| 120520 | AA258601 | Hs.161731 | EST | 2.4 |
| 120535 | BE350244 | Hs.96547 | ESTs | 2.5 |
| 120551 | AA279160 | Hs.111407 | Homo sapiens, clone IMAGE: 3613029, mRNA, partial cds | 5.2 |
| 120570 | AA280679 | Hs.271445 | ESTs, Weakly similar to ALU1_HUMAN ALU SUBFAMILY J SEQUENCE CONTAMINATION | 14.4 |
| 120582 | BE244830 | Hs.284228 | ZNF135-like protein | 10.2 |
| 120590 | AW372799 | Hs.125790 | leucine-rich repeat-containing 2 | 2.1 |
| 120596 | AA282074 | Hs.237323 | N-acetylglucosamine-phosphate mutase | 7.5 |
| 120619 | AW965339 | Hs.111471 | ESTs | 2.5 |
| 120624 | AW407987 | Hs.173518 | M-phase phosphoprotein homolog | 52.0 |
| 120639 | AA286942 | | gb: zs56f05.s1 NCI_CGAP_GCB1 Homo sapiens cDNA clone IMAGE: | 2.4 |
| | | | 701505 3′ similar to contains Alu | |
| 120648 | AA287095 | Hs.140309 | Homo sapiens, clone IMAGE: 3677194, mRNA, partial cds | 5.0 |
| 120653 | AW063659 | Hs.191649 | ESTs | 2.2 |
| 120668 | AW969638 | Hs.112318 | 6.2 kd protein | 2.2 |
| 120669 | BE536739 | Hs.109909 | ESTs | 1.9 |
| 120695 | AA976503 | | gb: oq30a04.s1 NCI_CGAP_GC4 Homo sapiens cDNA clone 3′ similar to contains PTR7.t1 PTR7 | 46.8 |
| 120696 | AI821539 | Hs.97249 | ESTs | 2.5 |
| 120713 | AW449855 | Hs.96557 | Homo sapiens cDNA FLJ12727 fis, clone NT2RP2000027 | 5.9 |
| 120718 | AA292747 | Hs.97296 | ESTs | 2.9 |
| 120750 | AI191410 | Hs.96693 | ESTs, Moderately similar to 2109260A B cell growth factor [H. sapiens] | 7.0 |
| 120774 | AI608909 | Hs.193985 | ESTs | 7.8 |
| 120807 | AA346385 | Hs.30002 | SH3-containing protein SH3GLB2; KIAA1848 protein | 6.8 |
| 120809 | AA346495 | | gb: EST52657 Fetal heart II Homo sapiens cDNA 3′ end similar to EST containing O family repeat, | 4.4 |
| 120938 | AA386260 | Hs.104632 | EST | 4.4 |
| 120977 | AA398155 | Hs.97600 | ESTs | 4.4 |
| 120984 | BE262951 | Hs.99052 | ESTs | 5.6 |
| 120985 | AI219896 | Hs.97592 | ESTs | 1.2 |
| 121011 | AA398360 | Hs.97608 | EST | 3.1 |
| 121026 | AI439713 | Hs.165295 | ESTs | 3.5 |
| 121081 | AA398721 | Hs.186749 | ESTs, Highly similar to I37550 mismatch repair protein MSH2 [H. sapiens] | 5.4 |
| 121133 | AA363307 | Hs.97032 | ESTs | 3.7 |
| 121176 | AL121523 | Hs.97774 | ESTs | 1.7 |
| 121223 | AI002110 | Hs.97169 | ESTs, Weakly similar to dJ667H12.2.1 [H. sapiens] | 2.9 |
| 121320 | AA403008 | Hs.301927 | c6.1A | 1.9 |
| 121340 | AW956981 | Hs.97910 | Homo sapiens cDNA FLJ13383 fis, clone PLACE1001024 | 3.5 |
| 121408 | AA406137 | Hs.98019 | EST | 6.0 |
| 121439 | AA410190 | Hs.98076 | ESTs, Weakly similar to A47582 B-cell growth factor precursor [H. sapiens] | 7.4 |
| 121450 | AA406430 | Hs.105362 | Homo sapiens, clone MGC: 18257, mRNA, complete cds | 6.9 |
| 121452 | AW971063 | Hs.292882 | ESTs | 1.8 |
| 121455 | H58306 | Hs.15165 | retinoic acid induced 14 | 10.5 |
| 121457 | W07404 | Hs.144502 | hypothetical protein FLJ22055 | 3.4 |
| 121496 | AA442224 | Hs.97900 | ESTs | 14.4 |
| 121505 | AA494172 | Hs.194417 | ESTs | 13.1 |
| 121508 | AA402515 | Hs.97887 | ESTs | 28.0 |
| 121513 | AA416653 | Hs.181510 | ESTs | 6.2 |
| 121514 | AA412112 | | gb: zt69b02.s1 Soares_testis_NHT Homo sapiens cDNA clone IMAGE: 727563 3′, mRNA sequence. | 2.6 |
| 121549 | AA412477 | Hs.98142 | EST | 7.4 |
| 121558 | AA412497 | | gb: zt95g12.s1 Soares_testis_NHT Homo sapiens cDNA clone IMAGE: 730150 3′ similar to contains | 2.8 |
| 121577 | AA411970 | Hs.98096 | EST | 3.5 |
| 121581 | AA416568 | | gb: zu05c10.s1 Soares_testis_NHT Homo sapiens cDNA clone 3′, mRNA sequence | 6.1 |
| 121589 | AD001528 | Hs.89718 | spermine synthase | 3.9 |
| 121594 | AA626010 | Hs.98247 | ESTs | 2.2 |
| 121622 | AA416931 | Hs.126065 | ESTs | 4.2 |
| 121655 | AA421537 | Hs.178072 | Homo sapiens mRNA; cDNA DKFZp434B1023 (from clone DKFZp434B1023) | 7.8 |
| 121682 | AA418160 | Hs.86043 | Homo sapiens cDNA FLJ13558 fis, clone PLACE1007743 | 2.0 |
| 121690 | AV660305 | Hs.110286 | ESTs | 4.7 |
| 121706 | U55184 | Hs.154145 | hypothetical protein FLJ11585 | 12.7 |
| 121714 | AA419225 | Hs.98269 | Homo sapiens cDNA FLJ11953 fis, clone HEMBB1000883 | 8.1 |
| 121729 | AI949597 | Hs.98325 | ESTs | 1.8 |
| 121731 | AA421041 | Hs.180744 | ESTs | 4.0 |
| 121744 | AA398784 | Hs.97514 | ESTs | 7.1 |
| 121748 | BE536911 | Hs.234545 | hypothetical protein NUF2R | 19.5 |
| 121773 | AB033022 | Hs.158654 | KIAA1196 protein | 7.9 |
| 121775 | AA421773 | Hs.161008 | ESTs | 1.7 |
| 121776 | AA292579 | Hs.125133 | hypothetical protein FLJ22501 | 6.6 |
| 121786 | AI810774 | Hs.98376 | ESTs | 10.5 |
| 121832 | AW340797 | Hs.98434 | ESTs | 5.8 |
| 121836 | AA328348 | Hs.218289 | ESTs | 3.8 |
| 121839 | AA425691 | Hs.191606 | ESTs, Highly similar to KIAA1048 protein [H. sapiens] | 5.0 |
| 121842 | AF027406 | Hs.104865 | serine/threonine kinase 23 | 2.7 |
| 121847 | AA446628 | Hs.2799 | cartilage linking protein 1 | 2.3 |
| 121871 | AW972668 | Hs.293044 | ESTs | 2.9 |
| 121882 | AA426376 | Hs.98459 | ESTs | 5.0 |
| 121911 | AA427950 | | gb: zw50f02.s1 Soares_total_fetus_Nb2HF8_9w Homo sapiens cDNA clone IMAGE: 773499 3′ | 7.2 |
| 121915 | AA428179 | Hs.223405 | ESTs, Moderately similar to A46010 X-linked retinopathy protein [H. sapiens] | 2.5 |
| 121935 | AA428647 | Hs.98611 | EST | 2.3 |
| 121983 | AA298760 | Hs.180191 | hypothetical protein FLJ14904 | 3.4 |
| 121985 | AI862570 | Hs.299214 | Homo sapiens, clone IMAGE: 2822295, mRNA, partial cds | 11.4 |
| 121995 | AA210863 | Hs.3532 | nemo-like kinase | 3.8 |
| 121999 | AA430211 | Hs.98668 | EST | 6.4 |
| 122009 | AW292763 | Hs.160822 | Homo sapiens cDNA: FLJ20863 fis, clone ADKA01804 | 2.2 |
| 122013 | AA431085 | Hs.98706 | ESTs | 6.5 |
| 122036 | W92142 | Hs.271963 | ESTs, Weakly similar to ALU5_HUMAN ALU SUBFAMILY SC SEQUENCE CONTAMINATION | 13.1 |
| 122050 | AI453076 | Hs.166109 | ELAV (embryonic lethal, abnormal vision, Drosophila)-like 2 | 9.1 |
| 122060 | AA431738 | Hs.98750 | EST | 13.1 |
| 122114 | AW161023 | Hs.104921 | ESTs | 1.5 |
| 122188 | AA398838 | | gb: zt80d01.r1 Soares_testis_NHT Homo sapiens cDNA clone 5′, mRNA sequence | 3.3 |
| 122204 | AA435936 | Hs.98842 | EST | 5.6 |
| 122246 | AA329550 | Hs.29417 | HCF-binding transcription factor Zhangfei | 5.1 |
| 122257 | AA436819 | Hs.98899 | ESTs | 5.6 |
| 122302 | AA441801 | Hs.104947 | ESTs | 5.8 |
| 122341 | AW601969 | Hs.99010 | hypothetical protein FLJ22263 similar to nuclear receptor-binding SET-domain protein 1 | 2.0 |
| 122356 | AA443794 | Hs.98390 | ESTs | 7.3 |
| 122369 | AA443985 | Hs.303222 | ESTs | 12.2 |
| 122371 | AA868555 | Hs.178222 | ESTs | 5.0 |
| 122372 | AA446008 | Hs.336677 | EST | 7.6 |
| 122378 | AB032948 | Hs.21356 | hypothetical protein DKFZp762K2015 | 2.5 |
| 122405 | AA446572 | Hs.303223 | EST | 2.8 |
| 122412 | AA446869 | Hs.119316 | ESTs | 7.3 |
| 122415 | AA446918 | Hs.99088 | EST | 1.9 |
| 122418 | AA446966 | Hs.99090 | ESTs, Moderately similar to similar to KIAA0766 [H. sapiens] | 6.8 |
| 122440 | AW505139 | Hs.9460 | Homo sapiens mRNA; cDNA DKFZp547C244 (from clone DKFZp547C244) | 2.6 |
| 122446 | AA447603 | Hs.99123 | EST | 1.8 |
| 122448 | AA447626 | Hs.99127 | EST | 3.5 |
| 122458 | AI266159 | Hs.104980 | ESTs | 1.5 |
| 122460 | AW418788 | Hs.99148 | ESTs, Weakly similar to S43569 R01H10.6 protein - Caenorhabditis elegans [C. elegans] | 9.7 |
| 122464 | AA448158 | Hs.99152 | EST | 4.8 |
| 122490 | AA448349 | Hs.238151 | EST | 6.1 |
| 122492 | AA448417 | Hs.104990 | ESTs | 5.4 |
| 122502 | AA204969 | Hs.234863 | Homo sapiens cDNA FLJ12082 fis, clone HEMBB1002492 | 1.3 |
| 122510 | AA449232 | Hs.99195 | ESTs | 11.2 |
| 122530 | AW959741 | Hs.40368 | adaptor-related protein complex 1, sigma 2 subunit | 10.1 |
| 122547 | AA779725 | Hs.164589 | ESTs | 2.5 |
| 122555 | AA194055 | Hs.293858 | ESTs | 1.9 |
| 122570 | AA452578 | Hs.262907 | ESTs | 9.5 |
| 122572 | AA452601 | Hs.99287 | EST | 11.0 |
| 122586 | AK001910 | Hs.99303 | Homo sapiens cDNA FLJ11048 fis, clone PLACE1004516 | 3.4 |
| 122587 | AB040893 | Hs.6968 | KIAA1460 protein | 2.0 |
| 122598 | AI028173 | Hs.99329 | ESTs | 1.7 |
| 122599 | AL355841 | Hs.99330 | hypothetical protein FLJ23588 | 4.4 |
| 122602 | AA411925 | Hs.301960 | ESTs | 4.6 |
| 122607 | AA453518 | Hs.98023 | ESTs | 61.5 |
| 122614 | AA453630 | Hs.99339 | EST | 10.7 |
| 122616 | AA453638 | Hs.161873 | ESTs | 107.3 |
| 122617 | AI681535 | Hs.148135 | serine/threonine kinase 33 | 121.4 |
| 122618 | AA453641 | | gb: zx48e06.s1 Soares_testis_NHT Homo sapiens cDNA clone 3′, mRNA sequence | 31.1 |
| 122622 | AA453987 | Hs.144802 | ESTs | 5.6 |
| 122717 | AA456859 | Hs.178358 | ESTs | 8.5 |
| 122762 | AI376875 | Hs.105119 | ESTs | 10.4 |
| 122829 | AW204530 | Hs.99500 | ESTs | 81.8 |
| 122834 | AA461492 | Hs.99545 | Homo sapiens cDNA FLJ10658 fis, clone NT2RP2006052 | 3.6 |
| 122836 | AA460581 | Hs.290996 | ESTs | 4.5 |
| 122837 | AA461509 | Hs.293565 | ESTs, Weakly similar to putative p150 [H. sapiens] | 2.7 |
| 122838 | AA460584 | Hs.334386 | ESTs | 75.3 |
| 122854 | AA600235 | Hs.9625 | NIMA (never in mitosis gene a)-related kinase 6 | 7.7 |
| 122856 | AI929374 | Hs.75367 | Src-like-adapter | 5.8 |
| 122861 | AA335721 | Hs.119394 | ESTs | 1.3 |
| 122866 | BE539656 | Hs.283705 | ESTs | 4.1 |
| 122868 | AF005216 | Hs.115541 | Janus kinase 2 (a protein tyrosine kinase) | 5.3 |
| 122870 | AW576312 | Hs.318722 | Homo sapiens cDNA: FLJ21766 fis, clone COLF7179 | 9.9 |
| 122872 | AW081394 | Hs.97103 | ESTs | 5.3 |
| 122879 | AA769410 | Hs.128654 | ESTs | 13.9 |
| 122907 | AA470074 | Hs.169896 | ESTs | 11.5 |
| 122916 | AA470140 | Hs.229170 | EST | 1.7 |
| 122981 | AA478951 | Hs.105629 | ESTs | 5.0 |
| 123013 | AW968324 | Hs.17384 | ESTs | 15.4 |
| 123016 | AW338067 | Hs.323231 | Homo sapiens cDNA FLJ11946 fis, clone HEMBB1000709 | 2.8 |
| 123034 | AL359571 | Hs.44054 | ninein (GSK3B interacting protein) | 8.7 |
| 123072 | AI382600 | Hs.104308 | ESTs, Weakly similar to KIAA1395 protein [H. sapiens] | 8.8 |
| 123082 | AA485360 | Hs.105661 | ESTs | 3.9 |
| 123088 | AI343652 | Hs.105667 | ESTs | 3.8 |
| 123110 | AA486256 | Hs.193510 | EST | 7.4 |
| 123114 | BE304942 | Hs.265848 | myomegalin | 2.8 |
| 123131 | T52027 | Hs.271795 | ESTs, Weakly similar to I38022 hypothetical protein [H. sapiens] | 2.4 |
| 123132 | AI061582 | Hs.324179 | Homo sapiens cDNA FLJ12371 fis, clone MAMMA1002434 | 15.6 |
| 123136 | AW451999 | Hs.194024 | ESTs | 5.1 |
| 123149 | AI734179 | Hs.105676 | ESTs | 23.8 |
| 123152 | AW601773 | Hs.270259 | ESTs | 5.2 |
| 123258 | AA490929 | Hs.105274 | ESTs, Weakly similar to RMS1_HUMAN REGULATOR OF | 9.3 |
| | | | MITOTIC SPINDLE ASSEMBLY 1 [H. sapiens] | |
| 123315 | AA496369 | | gb: zv37d10.s1 Soares ovary tumor NbHOT Homo sapiens cDNA clone IMAGE: 755827 3′ similar to | 4.1 |
| 123369 | AA504757 | Hs.105738 | ESTs | 6.9 |
| 123394 | AA731404 | Hs.105510 | ESTs | 3.6 |
| 123433 | AW450922 | Hs.112478 | ESTs | 3.7 |
| 123466 | AA599042 | Hs.112503 | EST | 7.4 |
| 123470 | AW303285 | Hs.303632 | Human DNA sequence from clone RP11-110H4 on chromosome 5 Contains a pseudogene similar to | 3.5 |
| 123471 | AB021644 | Hs.197219 | zinc finger protein 14 (KOX 6) | 5.2 |
| 123475 | BE439553 | Hs.250528 | Homo sapiens, clone IMAGE: 4098694, mRNA, partial cds | 1.7 |
| 123482 | N95059 | Hs.55098 | ESTs | 1.6 |
| 123486 | BE019072 | Hs.334802 | Homo sapiens cDNA FLJ14680 fis, clone NT2RP2004242, weakly similar to | 2.4 |
| 123508 | AW380388 | Hs.155546 | KIAA1080 protein; Golgi-associated, gamma-adaptin ear containing, ARF-binding protein 2 | 2.2 |
| 123615 | AA609170 | | gb: af12a12.s1 Soares_testis_NHT Homo sapiens cDNA clone 3′, mRNA sequence | 7.8 |
| 123619 | AA602964 | | gb: no97c02.s1 NCI_CGAP_Pr2 Homo sapiens cDNA clone, mRNA sequence | 2.8 |
| 123658 | AA609364 | | gb: zu71d09.s1 Soares_testis_NHT Homo sapiens cDNA clone IMAGE: | 1.7 |
| | | | 743441 3′ similar to contains Alu. | |
| 123674 | AI269609 | Hs.105187 | kinesin protein 9 gene | 5.7 |
| 123735 | NM_013241 | Hs.95231 | FH1/FH2 domain-containing protein | 10.0 |
| 123738 | AA609891 | Hs.112777 | EST | 5.2 |
| 123753 | AA609955 | Hs.234961 | Huntingtin interacting protein E | 30.6 |
| 123804 | AA620464 | Hs.261915 | EST, Weakly similar to S65657 alpha-1C-adrenergic receptor splice form 2 [H. sapiens] | 2.1 |
| 123811 | AA620586 | | gb: ae60g05.s1 Stratagene lung carcinoma 937218 Homo sapiens cDNA clone IMAGE: 951320 3′ | 2.7 |
| 123951 | AB012922 | Hs.173043 | metastasis-associated 1-like 1 | 6.2 |
| 123983 | AJ272267 | Hs.146178 | choline dehydrogenase | 4.4 |
| 124001 | L42542 | Hs.75447 | raIA binding protein 1 | 7.0 |
| 124006 | AI147155 | Hs.270016 | ESTs | 8.1 |
| 124070 | AI950314 | Hs.154762 | HIV-1 rev binding protein 2 | 3.7 |
| 124074 | H05635 | Hs.294030 | topoisomerase-related function protein 4-2 | 1.2 |
| 124178 | BE463721 | Hs.97101 | putative G protein-coupled receptor | 3.1 |
| 124203 | AA372796 | Hs.269339 | ESTs, Weakly similar to AF161356 1 HSPC093 [H. sapiens] | 5.7 |
| 124352 | AA640891 | Hs.102406 | ESTs | 3.1 |
| 124375 | D87454 | Hs.192966 | KIAA0265 protein | 3.5 |
| 124385 | AI267847 | | gb: aq49a10.x1 Stanley Frontal NB pool 2 Homo sapiens cDNA clone similar to contains | 57.1 |
| 124390 | AA317338 | Hs.7535 | COBW-like protein | 2.8 |
| 124391 | AF155099 | Hs.279780 | NY-REN-18 antigen | 7.1 |
| 124417 | N34059 | | gb: yv28h09.s1 Soares fetal liver spleen 1NFLS Homo sapiens cDNA clone 3′ similar to contains Alu | 3.3 |
| 124428 | H13540 | Hs.82202 | ribosomal protein L17 | 2.9 |
| 124440 | AA532519 | Hs.129043 | Human DNA sequence from clone 989H11 on chromosome 22q13.1-13.2. Contains part of a | 7.8 |
| 124466 | R10084 | Hs.113319 | kinesin heavy chain member 2 | 2.6 |
| 124482 | N53935 | | gb: yv59d09.s1 Soares fetal liver spleen 1NFLS Homo sapiens cDNA clone 3′, mRNA sequence | 7.9 |
| 124498 | H79433 | Hs.268997 | ESTs | 7.8 |
| 124515 | AA669097 | Hs.109370 | ESTs | 3.3 |
| 124608 | N71076 | Hs.102800 | ESTs, Weakly similar to neuronal thread protein AD7c-NTP [H. sapiens] | 4.5 |
| 124631 | NM_014053 | Hs.270594 | FLVCR protein | 3.2 |
| 124634 | AI765123 | Hs.143671 | Homo sapiens cDNA FLJ13533 fis, clone PLACE1006371 | 5.8 |
| 124637 | AA160474 | Hs.75798 | hypothetical protein | 9.3 |
| 124642 | AW968856 | Hs.278569 | sorting nexin 17 | 3.5 |
| 124649 | N92593 | Hs.313054 | ESTs | 6.1 |
| 124656 | AW297702 | Hs.102915 | ESTs | 8.3 |
| 124661 | R48170 | Hs.78436 | EphB1 | 5.6 |
| 124683 | AA381661 | Hs.119878 | ESTs, Weakly similar to M3K9_HUMAN MITOGEN-ACTIVATED PROTEIN KINASE KINASE | 7.9 |
| 124712 | R09166 | Hs.191148 | ESTs | 5.7 |
| 124735 | R22952 | Hs.268685 | ESTs | 11.3 |
| 124761 | AA374756 | Hs.93560 | Homo sapiens mRNA for KIAA1771 protein, partial cds | 9.0 |
| 124768 | AW368528 | Hs.100855 | ESTs | 8.1 |
| 124775 | R41772 | Hs.100878 | ESTs | 4.9 |
| 124777 | R41933 | Hs.140237 | ESTs, Weakly similar to ALU1_HUMAN ALU SUBFAMILY J SEQUENCE | 2.8 |
| 124788 | R43543 | Hs.100912 | Homo sapiens cDNA: FLJ22726 fis, clone HSI15005 | 5.1 |
| 124809 | AL355722 | Hs.106875 | Homo sapiens EST from clone 35214, full insert | 4.2 |
| 124811 | R46068 | Hs.288912 | hypothetical protein FLJ22604 | 14.2 |
| 124812 | R47948 | Hs.188732 | ESTs | 7.9 |
| 124822 | AA418160 | Hs.86043 | Homo sapiens cDNA FLJ13558 fis, clone PLACE1007743 | 6.6 |
| 124825 | AA501669 | Hs.336693 | ESTs | 2.3 |
| 124833 | AW975868 | Hs.294100 | ESTs | 2.7 |
| 124857 | R63652 | Hs.137190 | ESTs | 2.3 |
| 124860 | R65763 | Hs.101477 | EST | 23.9 |
| 124863 | AI382555 | Hs.127950 | bromodomain-containing 1 | 2.0 |
| 124876 | AF135422 | Hs.27059 | GDP-mannose pyrophosphorylase A | 4.4 |
| 124878 | BE397530 | Hs.288057 | hypothetical protein FLJ22242 | 2.7 |
| 124902 | H37941 | Hs.101883 | ESTs | 5.7 |
| 124903 | AW296713 | Hs.221441 | ESTs | 32.4 |
| 124930 | AI076343 | Hs.173939 | ESTs, Weakly similar to ALUB_HUMAN !!!! ALU CLASS B WARNING ENTRY !!! [H. sapiens] | 22.8 |
| 124942 | R99978 | Hs.268892 | ESTs, Moderately similar to B34087 hypothetical protein [H. sapiens] | 6.1 |
| 124958 | AI078645 | Hs.431 | murine leukemia viral (bmi-1) oncogene homolog | 1.9 |
| 124980 | T40841 | Hs.98681 | ESTs | 4.5 |
| 125002 | T59338 | Hs.269463 | ESTs, Weakly similar to ALU1_HUMAN ALU SUBFAMILY J SEQUENCE CONTAMINATION | 4.9 |
| 125047 | T79815 | Hs.279793 | ESTs | 5.0 |
| 125051 | T79956 | Hs.100588 | EST | 135.3 |
| 125056 | T81310 | Hs.100592 | ESTs | 5.4 |
| 125101 | AI472068 | Hs.286236 | KIAA1856 protein | 5.6 |
| 125113 | T96595 | Hs.302270 | ESTs, Weakly similar to ALUF_HUMAN !!!! ALU CLASS F WARNING ENTRY !!! [H. sapiens] | 1.8 |
| 125115 | T97341 | | gb: ye57e05.s1 Soares fetal liver spleen 1NFLS Homo sapiens cDNA clone IMAGE: 121856 3′ similar to | 9.6 |
| 125125 | AI222382 | Hs.240767 | Human DNA sequence from clone RP1-12G14 on chromosome 6q24.1-25.2. | 1.5 |
| | | | Contains the 5′ end of the gene | |
| 125147 | W38150 | | Empirically selected from AFFX single probeset | 1.7 |
| 125161 | W44657 | Hs.144232 | EST | 10.7 |
| 125249 | AA630863 | Hs.131375 | ESTs, Moderately similar to ALUB_HUMAN !!!! ALU CLASS B WARNING ENTRY !!! [H. sapiens] | 1.3 |
| 125255 | AF098162 | Hs.118631 | timeless (Drosophila) homolog | 9.4 |
| 125279 | AW401809 | Hs.4779 | KIAA1150 protein | 1.5 |
| 125280 | AI123705 | Hs.106932 | ESTs | 8.0 |
| 125298 | AW972542 | Hs.289008 | Homo sapiens cDNA: FLJ21814 fis, clone HEP01068 | 1.5 |
| 125660 | AW292171 | Hs.23978 | scaffold attachment factor B | 5.9 |
| 125827 | NM_003403 | Hs.97496 | YY1 transcription factor | 1.2 |
| 125891 | U29589 | Hs.7138 | cholinergic receptor, muscarinic 3 | 6.4 |
| 126005 | AW409701 | Hs.1578 | baculoviral IAP repeat-containing 5 (survivin) | 14.3 |
| 126202 | AA157632 | Hs.272630 | vacuolar proton pump delta polypeptide | 2.4 |
| 126695 | AA643322 | Hs.172028 | a disintegrin and metalloproteinase domain 10 | 9.1 |
| 127050 | AW411066 | Hs.274351 | CGI-89 protein | 17.0 |
| 127274 | AW966158 | Hs.58582 | Homo sapiens cDNA FLJ12789 fis, clone NT2RP2001947 | 12.8 |
| 128355 | AW293012 | Hs.161623 | ESTs | 7.3 |
| 128493 | D87466 | Hs.240112 | KIAA0276 protein | 3.1 |
| 128493 | D87466 | Hs.240112 | KIAA0276 protein | 1.3 |
| 128522 | BE173977 | Hs.10098 | putative nucleolar RNA helicase | 9.4 |
| 128527 | AA504583 | Hs.101047 | transcription factor 3 (E2A immunoglobulin enhancer binding factors E12/E47) | 1.5 |
| 128528 | R39234 | Hs.251699 | ESTs, Weakly similar to IDN4-GGTR14 [H. sapiens] | 2.8 |
| 128595 | U31875 | Hs.272499 | short-chain alcohol dehydrogenase family member | 12.1 |
| 128599 | NM_015366 | Hs.102336 | Rho GTPase activating protein 8 | 2.3 |
| 128604 | AI879099 | Hs.102397 | GIOT-3 for gonadotropin inducible transcription repressor-3 | 1.3 |
| 128608 | BE267994 | Hs.102419 | zinc finger protein | 7.1 |
| 128625 | AB037841 | Hs.102652 | hypothetical protein ASH1 | 1.3 |
| 128629 | AL096748 | Hs.102708 | DKFZP434A043 protein | 3.2 |
| 128639 | AW582962 | Hs.102897 | CGI-47 protein | 2.0 |
| 128656 | AA458542 | Hs.10326 | coatomer protein complex, subunit epsilon | 1.4 |
| 128656 | AA458542 | Hs.10326 | coatomer protein complex, subunit epsilo | 1.3 |
| 128658 | BE397354 | Hs.324830 | diptheria toxin resistance protein required for diphthamide biosynthesis (Saccharomyces)-like 2 | 2.4 |
| 128670 | AA975486 | Hs.103441 | Homo sapiens, Similar to RIKEN cDNA 1700010L19 gene, clone MGC: 16214, mRNA, complete cds | 7.1 |
| 128691 | W27939 | Hs.103834 | hypothetical protein MGC5576 | 7.7 |
| 128696 | BE081143 | Hs.225977 | nuclear receptor coactivator 3 | 3.8 |
| 128700 | Y15221 | Hs.103982 | small inducible cytokine subfamily B (Cys-X-Cys), member 11 | 1.6 |
| 128714 | T85231 | Hs.179661 | tubulin, beta 5 | 7.6 |
| 128717 | AK001564 | Hs.104222 | hypothetical protein FLJ10702 | 5.5 |
| 128733 | BE147740 | Hs.104558 | ESTs, Moderately similar to I38022 hypothetical protein [H. sapiens] | 2.7 |
| 128737 | AF292100 | Hs.104613 | RP42 homolog | 2.8 |
| 128742 | AA307211 | Hs.251531 | proteasome (prosome, macropain) subunit, alpha type, 4 | 4.4 |
| 128746 | AI470163 | Hs.323342 | actin related protein 2/3 complex, subunit 4 (20 kD) | 2.2 |
| 128747 | AB027249 | Hs.104741 | PDZ-binding kinase; T-cell originated protein kinase | 2.8 |
| 128772 | BE302796 | Hs.105097 | thymidine kinase 1, soluble | 5.3 |
| 128781 | N71826 | Hs.105465 | small nuclear ribonucleoprotein polypeptide F | 53.9 |
| 128797 | NM_002975 | Hs.105927 | stem cell growth factor; lymphocyte secreted C-type lectin | 13.3 |
| 128806 | AW630942 | Hs.106061 | RD RNA-binding protein | 2.6 |
| 128814 | AW248431 | Hs.256526 | nuclear prelamin A recognition factor | 2.2 |
| 128830 | BE281170 | Hs.106357 | valosin-containing protein | 5.9 |
| 128835 | AK001731 | Hs.106390 | Homo sapiens mRNA; cDNA DKFZp586H0924 (from clone DKFZp586H0924) | 1.6 |
| 128854 | BE159181 | Hs.168232 | hypothetical protein FLJ13855 | 2.2 |
| 128854 | BE159181 | Hs.168232 | hypothetical protein FLJ13855 | 1.9 |
| 128868 | AA419008 | Hs.106730 | chromosome 22 open reading frame 3 | 3.0 |
| 128868 | AA419008 | Hs.106730 | chromosome 22 open reading frame 3 | 2.2 |
| 128871 | AF189723 | Hs.106778 | ATPase, Ca++ transporting, type 2C, member 1 | 1.5 |
| 128891 | F34856 | Hs.292457 | Homo sapiens, clone MGC: 16362, mRNA, complete cds | 13.3 |
| 128906 | R57988 | Hs.10706 | epithelial protein lost in neoplasm beta | 4.7 |
| 128920 | AA622037 | Hs.166468 | programmed cell death 5 | 1.4 |
| 128925 | R67419 | Hs.21851 | Homo sapiens cDNA FLJ12900 fis, clone NT2RP2004321 | 1.9 |
| 128946 | Y13153 | Hs.107318 | kynurenine 3-monooxygenase (kynurenine 3-hydroxylase) | 7.2 |
| 128949 | AA009647 | Hs.8850 | a disintegrin and metalloproteinase domain 12 (meltrin alpha) (ADAM-12) | 2.4 |
| 128958 | AW139032 | Hs.107376 | hypothetical protein DKFZp434N035 | 1.3 |
| 128959 | AI580127 | Hs.107381 | hypothetical protein FLJ11200 | 10.9 |
| 128965 | AW150697 | Hs.107418 | ESTs | 1.4 |
| 128970 | AI375672 | Hs.165028 | ESTs | 1.3 |
| 128975 | BE560779 | Hs.284233 | NICE-5 protein | 14.0 |
| 128979 | AW271217 | Hs.281434 | Homo sapiens cDNA FLJ14028 fis, clone HEMBA1003838 | 1.6 |
| 128995 | AI816224 | Hs.107747 | DKFZP566C243 protein | 1.9 |
| 129019 | AI950087 | | gb: wq05c02.x1 NCI_CGAP_Kid12 Homo sapiens cDNA clone 3′, mRNA sequence | 2.9 |
| 129021 | AL044675 | Hs.173081 | KIAA0530 protein | 3.8 |
| 129021 | AL044675 | Hs.173081 | KIAA0530 protein | 2.5 |
| 129032 | R80088 | Hs.108104 | ubiquitin-conjugating enzyme E2L 3 | 3.4 |
| 129076 | AW296806 | Hs.326234 | ESTs, Highly similar to T46422 hypothetical protein DKFZp434M2023.1 [H. sapiens] | 5.0 |
| 129078 | AI351010 | Hs.102267 | lysosomal | 2.1 |
| 129088 | AA744610 | Hs.194431 | palladin | 17.1 |
| 129095 | L12350 | Hs.108623 | thrombospondin 2 | 2.7 |
| 129096 | AA463189 | Hs.288906 | WW Domain-Containing Gene | 20.9 |
| 129097 | BE243933 | Hs.108642 | zinc finger protein 22 (KOX 15) | 3.0 |
| 129099 | AF146074 | Hs.108660 | ATP-binding cassette, sub-family C (CFTR/MRP), member 5 | 5.8 |
| 129136 | W93048 | Hs.250723 | hypothetical protein MGC2747 | 5.9 |
| 129149 | AA356620 | Hs.108947 | KIAA0050 gene product | 6.3 |
| 129172 | AW162916 | Hs.241576 | hypothetical protein PRO2577 | 1.8 |
| 129192 | AA286914 | Hs.183299 | ESTs | 2.1 |
| 129194 | AA150797 | Hs.109276 | latexin protein | 3.2 |
| 129198 | N57532 | Hs.109315 | KIAA1415 protein | 5.8 |
| 129207 | AI934365 | Hs.109439 | osteoglycin (osteoinductive factor, mimecan) | 8.0 |
| 129228 | U40714 | Hs.239307 | tyrosyl-tRNA synthetase | 2.9 |
| 129229 | AF013758 | Hs.109643 | polyadenylate binding protein-interacting protein 1 | 3.2 |
| 129254 | AA252468 | Hs.1098 | DKFZp434J1813 protein | 2.6 |
| 129255 | AI961727 | Hs.109804 | H1 histone family, member X | 7.3 |
| 129288 | W26392 | Hs.110080 | ESTs, Weakly similar to S13495 pregnancy zone protein [H. sapiens] | 9.6 |
| 129296 | AI051967 | Hs.110122 | ESTs | 1.2 |
| 129323 | AA287239 | Hs.5518 | Home sapiens cDNA FLJ11311 fis, clone PLACE1010102 | 5.1 |
| 129340 | H75334 | Hs.11050 | F-box only protein 9 | 4.6 |
| 129347 | BE614192 | Hs.279869 | melanoma-associated antigen recognised by cytotoxic T lymphocytes | 7.6 |
| 129362 | U30246 | Hs.110736 | solute carrier family 12 (sodium/potassium/chloride transporters), member 2 | 6.7 |
| 129366 | BE220806 | Hs.184697 | Homo sapiens clone 23785 mRNA sequence | 8.6 |
| 129370 | AI686379 | Hs.110796 | SAR1 protein | 1.4 |
| 129372 | NM_016039 | Hs.110803 | CGI-99 protein | 2.0 |
| 129403 | AF149785 | Hs.111126 | pituitary tumor-transforming 1 interacting protein | 7.4 |
| 129404 | AI267700 | Hs.317584 | ESTs | 5.0 |
| 129404 | AI267700 | Hs.317584 | ESTs | 2.5 |
| 129423 | AA204686 | Hs.234149 | hypothetical protein FLJ20647 | 10.2 |
| 129449 | AI096988 | Hs.111554 | ADP-ribosylation factor-like 7 | 8.0 |
| 129453 | AW974265 | Hs.111632 | Lsm3 protein | 3.2 |
| 129482 | AA188185 | Hs.289043 | spindlin | 6.7 |
| 129482 | AA188185 | Hs.289043 | spindlin | 3.6 |
| 129513 | AW843633 | Hs.306163 | hypothetical protein AL110115 | 7.1 |
| 129515 | AF255303 | Hs.112227 | membrane-associated nucleic acid binding protein | 2.5 |
| 129527 | AA769221 | Hs.270847 | delta-tubulin | 3.2 |
| 129559 | W01296 | Hs.11360 | hypothetical protein FLJ14784 | 7.5 |
| 129560 | AA317841 | Hs.7845 | hypothetical protein MGC2752 | 6.8 |
| 129570 | AI923097 | Hs.11441 | chromosome 1 open reading frame 8 | 2.0 |
| 129575 | F08282 | Hs.278428 | progestin induced protein | 1.6 |
| 129587 | H14718 | Hs.11506 | Human clone 23589 mRNA sequence | 6.8 |
| 129588 | BE408300 | Hs.301862 | postmeiotic segregation increased 2-like 9 | 1.4 |
| 129591 | N57423 | Hs.179898 | HSPC055 protein | 7.3 |
| 129594 | AW403724 | Hs.36989 | coagulation factor VII (serum prothrombin conversion accelerator) | 9.0 |
| 129596 | AF035537 | Hs.115521 | REV3 (yeast homolog)-like, catalytic subunit of DNA polymerase zeta | 1.6 |
| 129628 | U38945 | Hs.1174 | cyclin-dependent kinase inhibitor 2A (melanoma, p16, inhibits CDK4) | 2.2 |
| 129628 | U38945 | Hs.1174 | cyclin-dependent kinase inhibitor 2A (me | 1.4 |
| 129629 | AK000398 | Hs.11747 | hypothetical protein FLJ20391 | 3.8 |
| 129649 | AD000092 | Hs.16488 | calreticulin | 3.3 |
| 129675 | NM_015556 | Hs.172180 | KIAA0440 protein | 13.4 |
| 129680 | U03749 | | gb: Human chromogranin A (CHGA) gene, promoter an | 14.1 |
| 129689 | AW748482 | Hs.77873 | B7 homolog 3 | 2.6 |
| 129702 | AI304966 | Hs.12035 | ESTs, Weakly similar to I38022 hypothetical protein [H. sapiens] | 7.4 |
| 129720 | AA156214 | Hs.12152 | APMCF1 protein | 2.0 |
| 129721 | NM_001415 | Hs.211539 | eukaryotic translation initiation factor 2, subunit 3 (gamma, 52 kD) | 1.7 |
| 129726 | H15474 | Hs.132898 | fatty acid desaturase 1 | 8.3 |
| 129778 | AK001676 | Hs.12457 | hypothetical protein FLJ10814 | 1.8 |
| 129779 | AA394090 | Hs.12460 | Homo sapiens clone 23870 mRNA sequence | 5.4 |
| 129800 | AF052112 | Hs.12540 | lysosomal | 1.7 |
| 129806 | AB023148 | Hs.173373 | KIAA0931 protein | 1.2 |
| 129815 | BE565817 | Hs.26498 | hypothetical protein FLJ21657 | 3.1 |
| 129840 | NM_006590 | Hs.12820 | SnRNP assembly defective 1 homolog | 1.8 |
| 129861 | AL049999 | Hs.85963 | DKFZP564M182 protein | 2.2 |
| 129864 | AI393237 | Hs.129914 | runt-related transcription factor 1 (acute myeloid leukemia 1; aml1 oncogene) | 1.7 |
| 129869 | AI222069 | Hs.13015 | hypothetical protein similar to mouse Dnajl1 | 2.7 |
| 129922 | AF042379 | Hs.13386 | gamma-tubulin complex protein 2 | 4.5 |
| 129945 | BE514376 | Hs.165998 | PAl-1 mRNA-binding protein | 1.8 |
| 129953 | AA412195 | Hs.13740 | ESTs | 2.5 |
| 129972 | AW753185 | Hs.180628 | dynamin 1-like | 1.8 |
| 129983 | U09848 | Hs.132390 | zinc finger protein 36 (KOX 18) | 1.3 |
| 129989 | AB015856 | Hs.247433 | activating transcription factor 6 | 4.0 |
| 130010 | AA301116 | Hs.142838 | nucleolar phosphoprotein Nopp34 | 1.6 |
| 130081 | AA287325 | Hs.14713 | ESTs | 4.0 |
| 130082 | S73265 | Hs.1473 | gastrin-releasing peptide | 1.8 |
| 130097 | AL046962 | Hs.14845 | forkhead box O3A | 2.8 |
| 130100 | AL135561 | Hs.14891 | hypothetical protein FLJ21047 | 2.3 |
| 130111 | X53002 | Hs.149846 | integrin, beta 5 | 2.3 |
| 130112 | AA916785 | Hs.180610 | splicing factor proline/glutamine rich (polypyrimidine tract-binding protein-associated) | 3.0 |
| 130112 | AA916785 | Hs.180610 | splicing factor proline/glutamine rich ( | 2.1 |
| 130128 | L76937 | Hs.150477 | Werner syndrome | 1.8 |
| 130135 | AA311426 | Hs.21635 | tubulin, gamma 1 | 6.1 |
| 130211 | NM_003358 | Hs.23703 | ESTs, Moderately to CEGT_HUMAN CERAMIDE GLUCOSYLTRANSFERASE [H. sapiens] | 1.6 |
| 130212 | D80001 | Hs.152629 | KIAA0179 protein | 1.3 |
| 130236 | R85367 | Hs.51957 | splicing factor, arginine/serine-rich 2, interacting protein | 2.0 |
| 130241 | AL035588 | Hs.153203 | MyoD family inhibitor | 3.2 |
| 130242 | X79201 | Hs.153221 | synovial sarcoma, translocated to X chromosome | 5.4 |
| 130249 | D81983 | Hs.322852 | GAS2-related on chromosome 22 | 4.8 |
| 130263 | NM_002497 | Hs.153704 | NIMA (never in mitosis gene a)-related kinase 2 | 1.4 |
| 130287 | AA479005 | Hs.154036 | tumor suppressing subtransferable candidate 3 | 2.6 |
| 130310 | AB011121 | Hs.154248 | amyotrophic lateral sclerosis 2 (juvenile) chromosome region, candidate 3 | 6.3 |
| 130353 | Z19084 | Hs.172210 | MUF1 protein | 6.2 |
| 130356 | AF127577 | Hs.155017 | nuclear receptor interacting protein 1 | 2.4 |
| 130357 | AJ224442 | Hs.155020 | putative methyltransferase | 3.4 |
| 130359 | NM_013449 | Hs.277401 | bromodomain adjacent to zinc finger domain, 2A | 8.5 |
| 130367 | AL135301 | Hs.8768 | hypothetical protein FLJ10849 | 1.4 |
| 130372 | AI077464 | Hs.5011 | RNA binding motif protein 9 | 3.3 |
| 130393 | N89487 | Hs.155291 | KIAA0005 gene product | 1.8 |
| 130399 | AW374106 | Hs.155356 | hypothetical protein MGC2840 similar to a putative glucosyltransferase | 3.4 |
| 130407 | BE385099 | Hs.334727 | hypothetical protein MGC3017 | 2.3 |
| 130409 | NM_001197 | Hs.155419 | BCL2-interacting killer (apoptosis-inducing) | 2.7 |
| 130419 | AF037448 | Hs.155489 | NS1-associated protein 1 | 1.8 |
| 130441 | U63630 | Hs.155637 | protein kinase, DNA-activated, catalytic polypeptide | 2.3 |
| 130448 | BE513202 | Hs.15589 | PPAR binding protein | 3.9 |
| 130455 | D90041 | Hs.155956 | N-acetyltransferase 1 (arylamine N-acetyltransferase) | 33.6 |
| 130455 | D90041 | Hs.155956 | N-acetyltransferase 1 (arylamine N-acety | 4.6 |
| 130471 | AL121438 | Hs.183706 | adducin 1 (alpha) | 2.7 |
| 130485 | BE245851 | Hs.180779 | H2B histone family, member B | 5.0 |
| 130487 | U49844 | Hs.77613 | ataxia telangiectasia and Rad3 related | 4.3 |
| 130498 | L38951 | Hs.180446 | karyopherin (importin) beta 1 | 1.6 |
| 130503 | BE208491 | Hs.295112 | KIAA0618 gene product | 16.1 |
| 130511 | L32137 | Hs.1584 | cartilage oligomeric matrix protein (pseudoachondroplasia, epiphyseal dysplasia 1, multiple) | 6.1 |
| 130511 | L32137 | Hs.1584 | cartilage oligomeric matrix protein (pse | 5.3 |
| 130526 | AW876523 | Hs.15929 | hypothetical protein FLJ12910 | 2.1 |
| 130542 | U64675 | Hs.179825 | RAN binding protein 2-like 1 | 7.8 |
| 130544 | AA321238 | Hs.4310 | eukaryotic translation initiation factor 1A | 1.5 |
| 130553 | AF062649 | Hs.252587 | pituitary tumor-transforming 1 | 14.4 |
| 130556 | AI907018 | Hs.15977 | Empirically selected from AFFX single probeset | 4.7 |
| 130567 | AA383092 | Hs.1608 | replication protein A3 (14 kD) | 7.9 |
| 130568 | AA232119 | Hs.16085 | putative G-protein coupled receptor | 3.3 |
| 130574 | AF083208 | Hs.16178 | apoptosis antagonizing transcription factor | 1.2 |
| 130586 | AB007891 | Hs.16349 | KIAA0431 protein | 5.6 |
| 130598 | AL042210 | Hs.16493 | hypothetical protein DKFZp762N2316; KIAA1803 protein | 1.4 |
| 130601 | AA609738 | Hs.16525 | ESTs | 1.5 |
| 130614 | AI354355 | Hs.16697 | down-regulator of transcription 1, TBP-binding (negative cofactor 2) | 1.3 |
| 130617 | M90516 | Hs.1674 | glutamine-fructose-6-phosphate transaminase 1 | 12.1 |
| 130617 | M90516 | Hs.1674 | glutamine-fructose-6-phosphate transamin | 2.4 |
| 130618 | AA383439 | Hs.16758 | Spir-1 protein | 15.9 |
| 130667 | BE246961 | Hs.17639 | Homo sapiens ubiquitin protein ligase (UBE3B) mRNA, partial cds | 13.9 |
| 130674 | AL048842 | Hs.194019 | attractin | 1.5 |
| 130675 | AA442233 | Hs.17731 | hypothetical protein FLJ12892 | 5.4 |
| 130692 | AA652501 | Hs.13561 | hypothetical protein MGC4692 | 5.0 |
| 130693 | R68537 | Hs.17962 | ESTs | 2.0 |
| 130712 | AJ271881 | Hs.279762 | bromodomain-containing 7 | 1.8 |
| 130714 | AI348274 | Hs.18212 | DNA segment on chromosome X (unique) 9879 expressed sequence | 2.0 |
| 130730 | AB007920 | Hs.18586 | KIAA0451 gene product | 3.7 |
| 130744 | H59696 | Hs.18747 | POP7 (processing of precursor, S. cerevisiae) homolog | 3.1 |
| 130751 | AF052105 | Hs.18879 | chromosome 12 open reading frame | 1.4 |
| 130757 | AL036067 | Hs.18925 | protein × 0001 | 5.7 |
| 130768 | AF258627 | Hs.211562 | ATP-binding cassette, sub-family A (ABC1), member 1 | 5.1 |
| 130789 | AK000355 | Hs.8899 | sirtuin (silent mating type information regulation 2, S. cerevisiae, homolog) 5 | 5.2 |
| 130815 | AB018298 | Hs.19822 | SEC24 (S. cerevisiae) related gene family, member D | 1.5 |
| 130836 | J05068 | Hs.2012 | transcobalamin I (vitamin B12 binding protein, R binder family) | 15.7 |
| 130841 | AL157468 | Hs.325825 | Homo sapiens cDNA FLJ20848 fis, clone ADKA01732 | 2.8 |
| 130843 | AA447492 | Hs.20183 | ESTs, Weakly similar to AF164793 1 protein × 013 [H. sapiens] | 1.5 |
| 130844 | U76248 | Hs.20191 | seven in absentia (Drosophila) homolog 2 | 3.4 |
| 130855 | AJ243706 | Hs.143323 | putative DNA/chromatin binding motif | 1.7 |
| 130861 | NM_016578 | Hs.20509 | HBV pX associated protein-8 | 1.9 |
| 130879 | NM_003416 | Hs.2076 | zinc finger protein 7 (KOX 4, clone HF.16) | 1.4 |
| 130880 | BE514434 | Hs.20830 | kinesin-like 2 | 2.1 |
| 130892 | AL120837 | Hs.20993 | high-glucose-regulated protein 8 | 2.4 |
| 130898 | AB033078 | Hs.186613 | sphingosine-1-phosphate lyase 1 | 1.7 |
| 130911 | BE409769 | Hs.21189 | DnaJ (Hsp40) homolog, subfamily A, member 2 | 1.8 |
| 130919 | N79110 | Hs.21276 | collagen, type IV, alpha 3 (Goodpasture antigen) binding protein | 2.3 |
| 130944 | BE382657 | Hs.21486 | signal transducer and activator of transcription 1, 91 kD | 5.4 |
| 130971 | N39842 | Hs.301444 | KIAA1673 | 2.2 |
| 130992 | BE398091 | Hs.74316 | desmoplakin (DPI, DPII) | 1.8 |
| 130993 | T97401 | Hs.21929 | ESTs | 1.6 |
| 131005 | AV658308 | Hs.2210 | thyroid hormone receptor interactor 3 | 1.6 |
| 131028 | AI879165 | Hs.2227 | CCAAT/enhancer binding protein (C/EBP), gamma | 1.2 |
| 131042 | AI826288 | Hs.171637 | hypothetical protein MGC2628 | 1.6 |
| 131046 | AA321649 | Hs.2248 | small inducible cytokine subfamily B (Cys-X-Cys), member 10 | 7.4 |
| 131046 | AA321649 | Hs.2248 | small inducible cytokine subfamily B (Cy | 3.0 |
| 131047 | H23230 | Hs.22481 | ESTs, Moderately similar to A46010 X-linked retinopathy protein [H. sapiens] | 1.7 |
| 131060 | AA194422 | Hs.22564 | myosin VI | 5.1 |
| 131060 | AA194422 | Hs.22564 | myosin VI | 2.5 |
| 131070 | N53344 | Hs.22607 | ESTs | 7.1 |
| 131076 | AA749230 | Hs.26433 | dolichyl-phosphate (UDP-N-acetylglucosamine) N-acetylglucosaminephosphotransferase 1 ( | 2.0 |
| 131076 | AA749230 | Hs.26433 | dolichyl-phosphate (UDP-N-acetylglucosam | 1.9 |
| 131099 | AL133353 | Hs.226581 | COX15 (yeast) homolog, cytochrome coxidase assembly protein | 7.0 |
| 131174 | NM_006540 | Hs.29131 | nuclear receptor coactivator 2 | 1.9 |
| 131185 | BE280074 | Hs.23960 | cyclin B1 | 5.8 |
| 131206 | AW138839 | Hs.24210 | ESTs | 2.0 |
| 131213 | AA885699 | Hs.24332 | CGI-26 protein | 7.0 |
| 131225 | H62087 | Hs.31659 | thyroid hormone receptor-associated protein, 95-kD subunit | 7.5 |
| 131231 | N47468 | Hs.59757 | zinc finger protein 281 | 2.9 |
| 131233 | D89053 | Hs.268012 | fatty-acid-Coenzyme A ligase, long-chain 3 | 3.5 |
| 131243 | AW383256 | Hs.24752 | spectrin SH3 domain binding protein 1 | 2.8 |
| 131245 | AL080080 | Hs.24766 | thioredoxin domain-containing | 2.8 |
| 131247 | AL043100 | Hs.326190 | fatty acid amide hydrolase | 5.6 |
| 131281 | AA251716 | Hs.25227 | ESTs | 5.7 |
| 131283 | X80038 | Hs.339713 | Homo sapiens clone F19374 APO E-C2 gene cluster | 1.3 |
| 131305 | AV656017 | Hs.184325 | CGI-76 protein | 5.0 |
| 131320 | AA505691 | Hs.145696 | splicing factor (CC1.3) | 1.8 |
| 131339 | AF058696 | Hs.25812 | Nijmegen breakage syndrome 1 (nibrin) | 2.6 |
| 131339 | AF058696 | Hs.25812 | Nijmegen breakage syndrome 1 (nibrin) | 2.6 |
| 131375 | AW293165 | Hs.143134 | ESTs | 5.4 |
| 131390 | BE269388 | Hs.182698 | mitochondrial ribosomal protein L20 | 5.3 |
| 131410 | BE259110 | Hs.279836 | HSPC166 protein | 2.2 |
| 131412 | NM_012247 | Hs.124027 | SELENOPHOSPHATE SYNTHETASE; Human selenium donor protein | 2.0 |
| 131429 | AL046302 | Hs.26750 | hypothetical protein FLJ21908 | 1.4 |
| 131458 | BE297567 | Hs.27047 | hypothetical protein FLJ20392 | 1.7 |
| 131475 | AA992841 | Hs.27263 | KIAA1458 protein | 2.0 |
| 131501 | AV661958 | Hs.8207 | GK001 protein | 2.6 |
| 131501 | AV661958 | Hs.8207 | GK001 protein | 1.6 |
| 131511 | AA732153 | Hs.27865 | Home sapiens cDNA: FLJ21333 fis, clone COL02535 | 2.0 |
| 131528 | AU076408 | Hs.28309 | UDP-glucose dehydrogenase | 1.6 |
| 131532 | BE268278 | Hs.28393 | hypothetical protein MGC2592 | 7.4 |
| 131543 | AW966881 | Hs.41639 | programmed cell death 2 | 2.2 |
| 131544 | AL355715 | Hs.28555 | programmed cell death 9 (PDCD9) | 2.1 |
| 131562 | NM_003512 | Hs.28777 | H2A histone family, member L | 1.7 |
| 131564 | T93500 | Hs.28792 | Homo sapiens cDNA FLJ11041 fis, clone PLACE1004405 | 5.1 |
| 131564 | T93500 | Hs.28792 | Homo sapiens cDNA FLJ11041 fis, clone PL | 1.8 |
| 131569 | AL389951 | Hs.271623 | nucleoporin 50 kD | 5.0 |
| 131618 | BE393822 | Hs.29645 | Homo sapiens mRNA; cDNA DKFZp761C029 (from clone DKFZp761C029); partial cds | 1.8 |
| 131622 | R78195 | Hs.29692 | Homo sapiens cDNA FLJ11436 fis, clone HEMBA1001213 | 1.3 |
| 131623 | AB037791 | Hs.29716 | hypothetical protein FLJ10980 | 2.2 |
| 131623 | AB037791 | Hs.29716 | hypothetical protein FLJ10980 | 1.9 |
| 131643 | AW410601 | Hs.30026 | HSPC182 protein | 2.9 |
| 131653 | AW960597 | Hs.30164 | ESTs | 1.3 |
| 131656 | AI218918 | Hs.30209 | KIAA0854 protein | 2.8 |
| 131669 | X52486 | Hs.3041 | uracil-DNA glycosylase 2 | 2.8 |
| 131692 | BE559681 | Hs.30736 | KIAA0124 protein | 5.6 |
| 131714 | AA642831 | Hs.31016 | putative DNA binding protein | 2.9 |
| 131722 | D13757 | Hs.311 | phosphoribosyl pyrophosphate amidotransferase | 3.4 |
| 131737 | AK001641 | Hs.31323 | inhibitor of kappa light polypeptide gene enhancer in B-cells, kinase complex-associated protein | 3.8 |
| 131760 | X76732 | Hs.3164 | nucleobindin 2 | 2.9 |
| 131760 | X76732 | Hs.3164 | nucleobindin 2 | 2.8 |
| 131763 | AI878932 | Hs.317 | topoisomerase (DNA) \| | 3.4 |
| 131772 | AA382590 | Hs.170980 | KIAA0948 protein | 25.5 |
| 131774 | BE267158 | Hs.169474 | DKFZP586J0119 protein | 5.5 |
| 131787 | D87077 | Hs.196275 | KIAA0240 protein | 2.4 |
| 131793 | AW966127 | Hs.32246 | Homo sapiens cDNA FLJ14656 fis, clone NT2RP2002439 | 7.9 |
| 131795 | BE501849 | Hs.32317 | high-mobility group 20B | 1.4 |
| 131798 | X86098 | Hs.301449 | adenovirus 5 E1A binding protein | 4.1 |
| 131817 | U20536 | Hs.3280 | caspase 6, apoptosis-related cysteine protease | 4.2 |
| 131824 | U28838 | Hs.32935 | TATA box binding protein (TBP)-associated factor, RNA polymerase III, GTF3B subunit 2 | 3.5 |
| 131850 | AI251317 | Hs.33184 | ESTs | 5.1 |
| 131878 | AA083764 | Hs.6101 | hypothetical protein MGC3178 | 5.8 |
| 131885 | BE502341 | Hs.3402 | ESTs | 13.7 |
| 131885 | BE502341 | Hs.3402 | ESTs | 2.4 |
| 131887 | W17064 | Hs.332848 | SWI/SNF related, matrix associated, actin dependent regulator of chromatin, subfamily e, member 1 | 3.2 |
| 131900 | AA099014 | Hs.231029 | Homo sapiens, clone MGC: 15961, mRNA, complete cds | 8.7 |
| 131900 | AA099014 | Hs.231029 | Homo sapiens, clone MGC: 15961, mRNA, com | 2.0 |
| 131904 | AF078866 | Hs.284296 | Homo sapiens cDNA: FLJ22993 fis, clone KAT11914 | 5.5 |
| 131905 | AA179298 | Hs.3439 | stomatin-like 2 | 11.3 |
| 131913 | AW207440 | Hs.185973 | degenerative spermatocyte (homolog Drosophila; lipid desaturase) | 1.7 |
| 131916 | AA025976 | Hs.34569 | ESTs | 5.2 |
| 131925 | AF151048 | Hs.183180 | anaphase promoting complex subunit 11 (yeast APC11 homolog) | 2.7 |
| 131929 | BE541211 | Hs.34804 | Homo sapiens cDNA FLJ11472 fis, clone HEMBA1001711 | 5.3 |
| 131941 | BE252983 | Hs.35086 | ubiquitin specific protease 1 | 2.3 |
| 131950 | AA355113 | Hs.35380 | × 001 protein | 1.5 |
| 131962 | AK000046 | Hs.267448 | hypothetical protein FLJ20039 | 2.3 |
| 131965 | W79283 | Hs.35962 | ESTs | 1.4 |
| 131971 | BE567100 | Hs.154938 | hypothetical protein MDS025 | 3.5 |
| 131977 | U90441 | Hs.3622 | procollagen-proline, 2-oxoglutarate 4-dioxygenase (proline 4-hydroxylase), alpha polypeptide II | 6.5 |
| 131985 | AA503020 | Hs.36563 | hypothetical protein FLJ22418 | 2.4 |
| 131991 | AF053306 | Hs.36708 | budding uninhibited by benzimidazoles 1 (yeast homolog), beta | 2.1 |
| 132019 | H56995 | Hs.37372 | Homo sapiens DNA binding peptide mRNA, partial cds | 3.2 |
| 132031 | AF193844 | Hs.3758 | COP9 complex subunit 7a | 5.8 |
| 132062 | BE266155 | Hs.3832 | clathrin-associated protein AP47 | 1.5 |
| 132084 | NM_002267 | Hs.3886 | karyopherin alpha 3 (importin alpha 4) | 3.7 |
| 132103 | BE171921 | Hs.3991 | ESTs | 1.4 |
| 132105 | AV646076 | Hs.39959 | ESTs | 5.8 |
| 132116 | AW960474 | Hs.40289 | ESTs | 1.7 |
| 132176 | AA857025 | Hs.8878 | kinesin-like 1 | 3.3 |
| 132180 | NM_004460 | Hs.418 | fibroblast activation protein, alpha | 14.7 |
| 132192 | AA206153 | Hs.4209 | mitochondrial ribosomal protein L37 | 5.5 |
| 132194 | R42432 | Hs.4212 | ESTs | 4.4 |
| 132203 | NM_004782 | Hs.194714 | synaptosomal-associated protein, 29 kD | 2.2 |
| 132207 | BE206939 | Hs.42287 | E2F transcription factor 6 | 2.2 |
| 132235 | AV658411 | Hs.42656 | KIAA1681 protein | 7.8 |
| 132240 | AB018324 | Hs.42676 | KIAA0781 protein | 1.5 |
| 132252 | AI566004 | Hs.141269 | Homo sapiens cDNA: FLJ21550 fis, clone COL06258 | 1.3 |
| 132266 | AA301228 | Hs.43299 | hypothetical protein FLJ12890 | 5.7 |
| 132273 | AA227710 | Hs.43658 | DKFZP586L151 protein | 4.2 |
| 132276 | AA653507 | Hs.285711 | hypothetical protein FLJ13089 | 2.1 |
| 132288 | N36110 | Hs.305971 | solute carrier family 2 (facilitated glucose transporter), member 10 | 1.5 |
| 132294 | AB023191 | Hs.44131 | KIAA0974 protein | 10.0 |
| 132298 | NM_015986 | Hs.7120 | cytokine receptor-like molecule 9 | 1.9 |
| 132299 | AW405882 | Hs.44205 | cortistatin | 9.2 |
| 132325 | N37065 | Hs.44856 | hypothetical protein FLJ12116 | 2.0 |
| 132348 | AW067708 | Hs.170311 | heterogeneous nuclear ribonucleoprotein D-like | 6.5 |
| 132370 | AW572805 | Hs.46645 | ESTs | 3.8 |
| 132374 | AF155582 | Hs.46744 | core1 UDP-galactose: N-acetylgalactosamine-alpha-R beta 1,3-galactosyltransferase | 1.5 |
| 132376 | AI279892 | Hs.46801 | sorting nexin 14 | 12.5 |
| 132384 | AA312135 | Hs.46967 | HSPCO34 protein | 28.3 |
| 132393 | AL135094 | Hs.47334 | hypothetical protein FLJ14495 | 1.9 |
| 132450 | AA100012 | Hs.48827 | hypothetical protein FLJ12085 | 1.9 |
| 132452 | AW973521 | Hs.247324 | mitochondrial ribosomal protein S14 | 6.1 |
| 132456 | AB011084 | Hs.48924 | KIAA0512 gene product; ALEX2 | 1.7 |
| 132465 | AW169847 | Hs.49169 | KIAA1634 protein | 8.6 |
| 132470 | AI224456 | Hs.4934 | H. sapiens polyA site DNA | 5.2 |
| 132484 | X16660 | Hs.119007 | RAB4, member RAS oncogene family | 1.4 |
| 132518 | AW885606 | Hs.5064 | ESTs | 6.1 |
| 132528 | T78736 | Hs.50758 | SMC4 (structural maintenance of chromosomes 4, yeast)-like 1 | 3.3 |
| 132530 | AA306105 | Hs.50785 | SEC22, vesicle trafficking protein (S. cerevisiae)-like 1 | 2.0 |
| 132532 | AA454132 | Hs.5080 | mitochondrial ribosomal protein L16 | 2.9 |
| 132534 | BE388673 | Hs.5086 | hypothetical protein MGC10433 | 2.2 |
| 132543 | BE568452 | Hs.5101 | protein regulator of cytokinesis 1 | 7.3 |
| 132571 | AW674699 | Hs.5169 | suppressor of G2 allele of SKP1, S. cerevisiae, homolog of | 1.7 |
| 132574 | AW631437 | Hs.5184 | TH1 drosophila homolog | 7.1 |
| 132596 | AK001484 | Hs.5298 | CGI-45 protein | 2.2 |
| 132611 | AA345547 | Hs.53263 | hypothetical protein FLJ13287 | 2.2 |
| 132612 | H12751 | Hs.5327 | PRO1914 protein | 6.8 |
| 132616 | BE262677 | Hs.283558 | hypothetical protein PRO1855 | 14.0 |
| 132638 | AI796870 | Hs.54277 | DNA segment on chromosome X (unique) 9928 expressed sequence | 11.4 |
| 132648 | U51127 | Hs.54434 | hypothetical protein MGC1715 | 1.9 |
| 132668 | AB018319 | Hs.5460 | KIAA0776 protein | 2.6 |
| 132692 | AW191962 | Hs.249239 | collagen, type VIII, alpha 2 | 2.0 |
| 132715 | F11875 | Hs.5534 | Homo sapiens cDNA FLJ12961 fis, clone NT2RP2005645 | 1.5 |
| 132718 | NM_004600 | Hs.554 | Sjogren syndrome antigen A2 (60 kD, ribonucleoprotein autoantigen SS-A/Ro) | 3.0 |
| 132724 | AI142265 | Hs.55498 | geranylgeranyl diphosphate synthase 1 | 2.4 |
| 132731 | AI189075 | Hs.301872 | hypothetical protein MGC4840 | 12.4 |
| 132744 | AA010233 | Hs.55921 | glutamyl-prolyl-tRNA synthetase | 14.6 |
| 132760 | AA125985 | Hs.56145 | thymosin, beta, identified in neuroblastoma cells | 2.7 |
| 132771 | Y10275 | Hs.56407 | phosphoserine phosphatase | 3.0 |
| 132773 | AA459713 | Hs.295901 | KIAA0493 protein | 2.3 |
| 132784 | AI142133 | Hs.56845 | GDP dissociation inhibitor 2 | 1.8 |
| 132798 | AI026701 | Hs.5716 | KIAA0310 gene product | 3.7 |
| 132807 | U07418 | Hs.57301 | mutL (E. coli) homolog 1 (colon cancer, nonpolyposis type 2) | 1.8 |
| 132810 | AB007944 | Hs.5737 | KIAA0475 gene product | 5.9 |
| 132813 | BE313625 | Hs.57435 | solute carrier family 11 (proton-coupled divalent metal ion transporters), member 2 | 8.7 |
| 132815 | AI815189 | Hs.57475 | sex comb on midleg homolog 1 | 6.4 |
| 132817 | N27852 | Hs.57553 | tousled-like kinase 2 | 3.6 |
| 132821 | AJ251595 | Hs.169610 | CD44 antigen (homing function and Indian blood group system) | 2.8 |
| 132833 | U78525 | Hs.57783 | eukaryotic translation initiation factor 3, subunit 9 (eta, 116 kD) | 14.6 |
| 132842 | NM_016154 | Hs.279771 | Homo sapiens clone PP1596 unknown mRNA | 1.6 |
| 132844 | F12200 | Hs.5811 | chromosome 21 open reading frame 59 | 2.5 |
| 132851 | U09716 | Hs.287912 | lectin, mannose-binding, 1 | 1.4 |
| 132863 | BE268048 | Hs.236494 | RAB10, member RAS oncogene family | 4.2 |
| 132869 | AW963217 | Hs.203961 | ESTs, Moderately similar to AF116721 89 PRO2168 [H. sapiens] | 2.8 |
| 132873 | AW007683 | Hs.58598 | KIAA1266 protein | 2.0 |
| 132875 | NM_004850 | Hs.58617 | Rho-associated, coiled-coil containing protein kinase 2 | 1.6 |
| 132891 | BE267143 | Hs.59271 | U2(RNU2) small nuclear RNA auxilliary factor 1 (non-standard symbol) | 1.4 |
| 132897 | AW503667 | Hs.59545 | ring finger protein 15 | 5.4 |
| 132902 | AI936442 | Hs.59838 | hypothetical protein FLJ10808 | 6.1 |
| 132912 | AW732760 | Hs.167578 | Homo sapiens cDNA FLJ11095 fis, clone PLACE1005374 | 7.1 |
| 132913 | W78714 | Hs.60257 | Homo sapiens cDNA FLJ13598 fis, clone PLACE 1009921 | 2.8 |
| 132940 | T79136 | Hs.127243 | Homo sapiens mRNA for KIAA1724 protein, partial cds | 6.1 |
| 132941 | AI817165 | Hs.6120 | hypothetical protein FLJ13222 | 10.3 |
| 132942 | AA554458 | Hs.197751 | KIAA0666 protein | 1.8 |
| 132952 | AI658580 | Hs.61426 | Homo sapiens mesenchymal stem cell protein DSC96 mRNA, partial cds | 2.2 |
| 132962 | AA576635 | Hs.6153 | CGI-48 protein | 4.9 |
| 132972 | AA034365 | Hs.288924 | Homo sapiens cDNA FLJ11392 fis, clone HEMBA1000575 | 2.7 |
| 132973 | AA035446 | Hs.323277 | ESTs | 5.3 |
| 132977 | AA093322 | Hs.301404 | RNA binding motif protein 3 | 3.2 |
| 132980 | AA040696 | Hs.62016 | ESTs | 1.3 |
| 132994 | AA112748 | Hs.279905 | clone HQ0310 PRO0310p1 | 3.0 |
| 133012 | AA847843 | Hs.62711 | Homo sapiens, clone IMAGE: 3351295, mRNA | 10.3 |
| 133015 | AJ002744 | Hs.246315 | UDP-N-acetyl-alpha-D-galactosamine: polypeptide N-acetylgalactosaminyltransferase 7 (GalNAc-T7) | 2.1 |
| 133016 | AI439688 | Hs.6289 | hypothetical protein FLJ20886 | 1.3 |
| 133053 | AI065016 | Hs.6390 | Homo sapiens clone FLB3344 PRO0845 mRNA, complete cds | 6.0 |
| 133062 | AW500374 | Hs.64056 | PRO0149 protein | 5.3 |
| 133069 | BE247441 | Hs.6430 | protein with polyglutamine repeat; calcium (ca2+) homeostasis endoplasmic reticulum protein | 4.9 |
| 133091 | AK001628 | Hs.64691 | KIAA0483 protein | 3.5 |
| 133110 | AA808177 | Hs.65228 | ESTs | 13.1 |
| 133134 | AF198620 | Hs.65648 | RNA binding motif protein 8A | 1.3 |
| 133145 | H94227 | Hs.6592 | Homo sapiens, clone IMAGE: 2961368, mRNA, partial cds | 2.2 |
| 133152 | Z11695 | Hs.324473 | mitogen-activated protein kinase 1 | 1.3 |
| 133174 | AA431620 | Hs.324178 | hypothetical protein MGC2745 | 17.1 |
| 133175 | AW955632 | Hs.66666 | ESTs, Weakly similar to S19560 proline-rich protein MP4 - mouse [M. musculus] | 1.8 |
| 133177 | X97795 | Hs.66718 | RAD54 (S. cerevisiae)-like | 4.9 |
| 133197 | AI275243 | Hs.180201 | hypothetical protein FLJ20671 | 3.1 |
| 133208 | AI801777 | Hs.6774 | ESTs | 4.4 |
| 133226 | AW954569 | Hs.296287 | Homo sapiens, Similar to bromodomain-containing 4, clone IMAGE: 3542455, mRNA, partial cds | 1.7 |
| 133228 | AI492924 | Hs.6831 | golgi phosphoprotein 1 | 6.0 |
| 133240 | AK001489 | Hs.242894 | ADP-ribosylation factor-like 1 | 1.5 |
| 133254 | AI567421 | Hs.273330 | Homo sapiens, clone IMAGE: 3544662, mRNA, partial cds | 1.4 |
| 133266 | AI160873 | Hs.69233 | zinc finger protein | 5.6 |
| 133268 | AW956781 | Hs.293937 | ESTs, Weakly similar to FXD2_HUMAN FORKHEAD BOX PROTEIN D2 [H. sapiens] | 1.9 |
| 133285 | M76477 | Hs.289082 | GM2 ganglioside activator protein | 4.7 |
| 133291 | BE297855 | Hs.69855 | NRAS-related gene | 5.0 |
| 133314 | AA102670 | Hs.70725 | gamma-aminobutyric acid (GABA) A receptor, pi | 2.7 |
| 133321 | T79526 | Hs.179516 | integral type I protein | 9.3 |
| 133327 | AL390127 | Hs.7104 | Kruppel-like factor 13 | 4.4 |
| 133347 | BE257758 | Hs.71475 | add cluster protein 33 | 1.8 |
| 133360 | AI016521 | Hs.71816 | v-akt murine thymoma viral oncogene homolog 1 | 5.5 |
| 133366 | AA292811 | Hs.72050 | non-metastatic cells 5, protein expressed in (nucleoside-diphosphate kinase) | 2.7 |
| 133367 | AF231919 | Hs.18759 | KIAA0539 gene product | 1.7 |
| 133370 | AF245505 | Hs.72157 | DKFZP564I1922 protein | 1.8 |
| 133383 | BE313555 | Hs.7252 | KIAA1224 protein | 1.7 |
| 133390 | AI950382 | Hs.72660 | phosphatidylserine receptor | 1.3 |
| 133391 | AW103364 | Hs.727 | inhibin, beta A (activin A, activin AB alpha polypeptide) | 16.1 |
| 133394 | AA305127 | Hs.237225 | hypothetical protein HT023 | 12.2 |
| 133437 | AL031591 | Hs.7370 | phosphotidylinositol transfer protein, beta | 10.4 |
| 133452 | NM_002759 | Hs.274382 | protein kinase, interferon-inducible double stranded RNA dependent | 1.2 |
| 133453 | AI659306 | Hs.73826 | protein tyrosine phosphatase, non-receptor type 4 (megakaryocyte) | 1.7 |
| 133500 | AW964804 | Hs.74280 | hypothetical protein FLJ22237 | 11.1 |
| 133529 | W45623 | Hs.74571 | ADP-ribosylation factor 1 | 2.8 |
| 133540 | AL037159 | Hs.74619 | proteasome (prosome, macropain) 26S subunit, non-ATPase, 2 | 2.9 |
| 133543 | AU077073 | Hs.108327 | damage-specific DNA binding protein 1 (127 kD) | 2.5 |
| 133578 | AU077050 | Hs.75066 | translin | 1.5 |
| 133579 | X75346 | Hs.75074 | mitogen-activated protein kinase-activated protein kinase 2 | 2.1 |
| 133582 | BE391579 | Hs.75087 | Fas-activated serine/threonine kinase | 1.3 |
| 133594 | AW160781 | Hs.172589 | nuclear phosphoprotein similar to S. cerevisiae PWP1 | 2.2 |
| 133595 | AA393273 | Hs.75133 | transcription factor 6-like 1 (mitochondrial transcription factor 1-like) | 1.5 |
| 133599 | NM_002885 | Hs.75151 | RAP1, GTPase activating protein 1 | 5.7 |
| 133621 | NM_004893 | Hs.75258 | H2A histone family, member Y | 25.5 |
| 133627 | NM_002047 | Hs.75280 | glycyl-tRNA synthetase | 15.8 |
| 133631 | NM_000401 | Hs.75334 | exostoses (multiple) 2 | 3.3 |
| 133649 | U25849 | Hs.75393 | acid phosphatase 1, soluble | 1.6 |
| 133690 | AV661185 | Hs.75574 | mitochondrial ribosomal protein L19 | 4.1 |
| 133720 | L27841 | Hs.75737 | pericentriolar material 1 | 1.5 |
| 133722 | AW969976 | Hs.279009 | matrix Gla protein | 6.3 |
| 133751 | AW402048 | Hs.334787 | Homo sapiens, Similar to likely ortholog of yeast ARV1, clone IMAGE: 3506392, mRNA | 3.9 |
| 133757 | T52946 | Hs.196209 | RAE1 (RNA export 1, S pombe) homolog | 1.7 |
| 133760 | BE271766 | Hs.181357 | laminin receptor 1 (67 kD, ribosomal protein SA) | 1.8 |
| 133765 | M62194 | Hs.75929 | cadherin 11, type 2, OB-cadherin (osteoblast) | 1.5 |
| 133780 | AA557660 | Hs.76152 | decorin | 3.5 |
| 133784 | BE622743 | Hs.301064 | arfaptin 1 | 6.8 |
| 133791 | M34338 | Hs.76244 | spermidine synthase | 2.6 |
| 133797 | AL133921 | Hs.76272 | retinoblastoma-binding protein 2 | 1.4 |
| 133822 | D50525 | Hs.699 | peptidylprolyl isomerase B (cyclophilin B) | 8.0 |
| 133842 | AW797468 | Hs.285013 | putative human HLA class II associated protein I | 13.5 |
| 133845 | AA147026 | Hs.76704 | ESTs | 2.2 |
| 133850 | W29092 | Hs.7678 | cellular retinoic acid-binding protein 1 | 1.8 |
| 133859 | U86782 | Hs.178761 | 26S proteasome-associated pad1 homolog | 2.0 |
| 133865 | AB011155 | Hs.170290 | discs, large (Drosophila) homolog 5 | 2.8 |
| 133867 | AW340125 | Hs.76989 | KIAA0097 gene product | 6.7 |
| 133868 | AB012193 | Hs.183874 | cullin 4A | 2.5 |
| 133881 | U30872 | Hs.77204 | centromere protein F (350/400 kD, mitosin) | 3.0 |
| 133922 | U30825 | Hs.77608 | splicing factor, arginine/serine-rich 9 | 1.4 |
| 133924 | D86326 | Hs.325948 | vesicle docking protein p115 | 5.4 |
| 133929 | NM_006306 | Hs.211602 | SMC1 (structural maintenance of chromosomes 1, yeast)-like 1 | 4.9 |
| 133936 | L17128 | Hs.77719 | gamma-glutamyl carboxylase | 3.7 |
| 133941 | BE244332 | Hs.77770 | adaptor-related protein complex 3, mu 2 subunit | 12.1 |
| 133959 | X81789 | Hs.77897 | splicing factor 3a, subunit 3, 60 kD | 9.7 |
| 133976 | AI908165 | Hs.169946 | GATA-binding protein 3 (T-cell receptor gene activator) | 3.1 |
| 133989 | AL040328 | Hs.78202 | SWI/SNF related, matrix associated, actin dependent regulator of chromatin | 1.3 |
| 133997 | AI824113 | Hs.78281 | regulator of G-protein signalling 12 | 9.7 |
| 134010 | AB016092 | Hs.197114 | RNA binding protein; AT-rich element binding factor | 2.4 |
| 134015 | D31764 | Hs.278569 | sorting nexin 17 | 2.5 |
| 134070 | NM_003590 | Hs.78946 | cullin 3 | 1.3 |
| 134110 | U41060 | Hs.79136 | LIV-1 protein, estrogen regulated | 4.2 |
| 134129 | NM_014742 | Hs.79305 | KIAA0255 gene product | 2.2 |
| 134134 | H86504 | Hs.173328 | protein phosphatase 2, regulatory subunit B (B56), epsilon isoform | 5.0 |
| 134200 | BE559598 | Hs.197803 | KIAA0160 protein | 3.2 |
| 134206 | AF107463 | Hs.79968 | splicing factor 30, survival of motor neuron-related | 2.5 |
| 134208 | NM_000288 | Hs.79993 | peroxisomal biogenesis factor 7 | 2.1 |
| 134219 | NM_000402 | Hs.80206 | glucose-6-phosphate dehydrogenase | 9.1 |
| 134234 | BE300078 | Hs.80449 | Homo sapiens, clone IMAGE: 3535294, mRNA, partial cds | 2.8 |
| 134275 | AI878910 | Hs.3688 | cisplatin resistance-associated overexpressed protein | 1.8 |
| 134292 | AI906291 | Hs.81234 | immunoglobulin superfamily, member 3 | 2.0 |
| 134301 | AW502505 | Hs.81360 | Homo sapiens cDNA: FLJ21927 fis, clone HEP04178, highly similar to HSU90909 | 2.5 |
| 134305 | U61397 | Hs.81424 | ubiquitin-like 1 (sentrin) | 2.8 |
| 134324 | AB029023 | Hs.179946 | KIAA1100 protein | 10.4 |
| 134326 | AW903838 | Hs.81800 | chondroitin sulfate proteoglycan 2 (versican) | 1.9 |
| 134329 | N92036 | Hs.81848 | RAD21 (S. pombe) homolog | 2.6 |
| 134337 | NM_004922 | Hs.81964 | SEC24 (S. cerevisiae) related gene family, member C | 2.3 |
| 134348 | AW291946 | Hs.82065 | interleukin 6 signal transducer (gp130, oncostatin M receptor) | 13.0 |
| 134367 | AA339449 | Hs.82285 | phosphoribosylglycinamide formyltransferase, phosphoribosylglycinamide synthetase, | 8.8 |
| 134376 | X06560 | Hs.82396 | 2′,5′-oligoadenylate synthetase 1 (40-46 kD) | 1.5 |
| 134379 | AW362124 | Hs.323193 | hypothetical protein MGC3222 | 8.1 |
| 134384 | AI589941 | Hs.8254 | Homo sapiens, Similar to tumor differentially expressed 1, clone IMAGE: 3639252, mRNA, partial cds | 2.6 |
| 134391 | AA417383 | Hs.82582 | integrin, beta-like 1 (with EGF-like repeat domains) | 4.1 |
| 134395 | AA456539 | Hs.8262 | lysosomal | 1.7 |
| 134403 | AA334551 | Hs.82767 | sperm specific antigen 2 | 2.6 |
| 134405 | AW067903 | Hs.82772 | collagen, type XI, alpha 1 | 1.3 |
| 134411 | BE272095 | Hs.167791 | reticulocalbin 1, EF-hand calcium binding domain | 3.2 |
| 134415 | AI750762 | Hs.82911 | protein tyrosine phosphatase type IVA, member 2 | 1.9 |
| 134421 | AU077196 | Hs.82985 | collagen, type V, alpha 2 | 10.3 |
| 134424 | Z44190 | Hs.83023 | peroxisomal biogenesis factor 11B | 2.4 |
| 134446 | AA112036 | Hs.83419 | KIAA0252 protein | 1.2 |
| 134447 | M58603 | Hs.83428 | nuclear factor of kappa light polypeptide gene enhancer in B-cells 1 (p105) | 1.6 |
| 134470 | X54942 | Hs.83758 | CDC28 protein kinase 2 | 2.1 |
| 134480 | NM_005000 | Hs.83916 | Empirically selected from AFFX single probeset | 5.3 |
| 134485 | X82153 | Hs.83942 | cathepsin K (pycnodysostosis) | 2.5 |
| 134498 | AW246273 | Hs.84131 | threonyl-tRNA synthetase | 2.1 |
| 134513 | AA425473 | Hs.84429 | KIAA0971 protein | 3.8 |
| 134516 | AK001571 | Hs.273357 | hypothetical protein FLJ10709 | 2.4 |
| 134520 | BE091005 | Hs.74861 | activated RNA polymerase II transcription cofactor 4 | 6.7 |
| 134529 | AW411479 | Hs.848 | FK506-binding protein 4 (59 kD) | 2.3 |
| 134577 | BE244323 | Hs.85951 | exportin, tRNA (nuclear export receptor for tRNAs) | 5.5 |
| 134582 | AA927177 | Hs.86041 | CGG triplet repeat binding protein 1 | 5.8 |
| 134612 | AW068223 | Hs.171581 | ubiquitin C-terminal hydrolase UCH37 | 2.2 |
| 134624 | AF035119 | Hs.8700 | deleted in liver cancer 1 | 2.0 |
| 134632 | X78520 | Hs.174139 | chloride channel 3 | 2.3 |
| 134654 | AK001741 | Hs.8739 | hypothetical protein FLJ10879 | 1.4 |
| 134664 | AA256106 | Hs.87507 | ESTs | 72.9 |
| 134666 | BE391929 | Hs.8752 | transmembrane protein 4 | 8.5 |
| 134687 | U62317 | Hs.88251 | arylsulfatase A | 6.0 |
| 134692 | NM_003474 | Hs.8850 | a disintegrin and metalloproteinase domain 12 (meltrin alpha) | 4.3 |
| 134705 | BE161887 | Hs.88799 | anaphase-promoting complex subunit 10 | 2.3 |
| 134714 | Y14768 | Hs.890 | lysosomal | 6.7 |
| 134719 | AA852985 | Hs.89232 | chromobox homolog 5 (Drosophila HP1 alpha) | 2.3 |
| 134722 | AF129536 | Hs.284226 | F-box only protein 6 | 2.9 |
| 134724 | AF045239 | Hs.321576 | ring finger protein 22 | 6.6 |
| 134746 | X07871 | Hs.89476 | CD2 antigen (p50), sheep red blood cell receptor | 2.3 |
| 134751 | AW630803 | Hs.89497 | lamin B1 | 6.2 |
| 134790 | BE002798 | Hs.287850 | integral membrane protein 1 | 1.9 |
| 134806 | AD001528 | Hs.89718 | spermine synthase | 1.8 |
| 134834 | AW451370 | Hs.8991 | adaptor-related protein complex 1, gamma 2 subunit | 1.4 |
| 134850 | AI701162 | Hs.90207 | hypothetical protein MGC11138 | 1.4 |
| 134853 | BE268326 | Hs.90280 | 5-aminoimidazole-4-carboxamide ribonucleotide formyltransferase/IMP cyclohydrolase | 5.6 |
| 134859 | D26488 | Hs.90315 | KIAA0007 protein | 2.8 |
| 134880 | AI879195 | Hs.90606 | 15 kDa selenoprotein | 1.7 |
| 134910 | AA532963 | Hs.9100 | Homo sapiens cDNA FLJ13100 fis, clone NT2RP3002255 | 1.7 |
| 134925 | AW885909 | Hs.6975 | PRO1073 protein | 2.1 |
| 134955 | AW401361 | Hs.91773 | protein phosphatase 2 (formerly 2A), catalytic subunit, alpha isoform | 1.3 |
| 134971 | AI097346 | Hs.286049 | phosphoserine aminotransferase | 2.1 |
| 134975 | R50333 | Hs.92186 | Leman coiled-coil protein | 2.3 |
| 135011 | AB037835 | Hs.92991 | KIAA1414 protein | 1.6 |
| 135022 | NM_000408 | Hs.93201 | glycerol-3-phosphate dehydrogenase 2 (mitochondrial) | 3.9 |
| 135032 | AW301984 | Hs.173685 | hypothetical protein FLJ12619 | 6.2 |
| 135077 | AW503733 | Hs.9414 | KIAA1488 protein | 2.0 |
| 135083 | AB036063 | Hs.94262 | p53-inducible ribonucleotide reductase small subunit 2 homolog | 1.3 |
| 135095 | AF027219 | Hs.9443 | zinc finger protein 202 | 7.1 |
| 135096 | AA081258 | Hs.132390 | zinc finger protein 36 (KOX 18) | 3.2 |
| 135153 | AI093155 | Hs.95420 | JM27 protein | 2.5 |
| 135181 | BE250865 | Hs.279529 | px19-like protein | 1.4 |
| 135199 | AA477514 | Hs.96247 | translin-associated factor X | 5.0 |
| 135207 | N26427 | Hs.9634 | ESTs, Highly similar to C10_HUMAN PUTATIVE C10 PROTEIN [H. sapiens] | 6.1 |
| 135214 | T78802 | Hs.96560 | hypothetical protein FLJ11656 | 4.6 |
| 135243 | BE463721 | Hs.97101 | putative G protein-coupled receptor | 5.6 |
| 135245 | AI028767 | Hs.262603 | ESTs | 3.5 |
| 135257 | AW291023 | Hs.97255 | ESTs, Weakly similar to A46010 X-linked retinopathy protein [H. sapiens] | 1.2 |
| 135263 | AI088775 | Hs.55498 | geranylgeranyl diphosphate synthase 1 | 2.6 |
| 135274 | AA448460 | Hs.112017 | GE36 gene | 5.3 |
| 135294 | AA150320 | Hs.9800 | protein kinase Njmu-R1 | 9.1 |
| 135295 | AI090838 | Hs.98006 | ESTs | 2.4 |
| 135307 | AI743770 | Hs.98368 | ESTs, Weakly similar to KIAA0822 protein [H. sapiens] | 13.3 |
| 135321 | AI652069 | Hs.98614 | ribosome binding protein 1 (dog 180 kD homolog) | 2.6 |
| 135354 | AA456454 | Hs.183418 | cell division cycle 2-like 1 (PITSLRE proteins) | 8.3 |
| 135361 | AA373452 | Hs.167700 | Homo sapiens cDNA FLJ10174 fis, clone HEMBA1003959 | 1.5 |
| 135389 | U05237 | Hs.99872 | fetal Alzheimer antigen | 4.9 |
| 135400 | X78592 | Hs.99915 | androgen receptor (dihydrotestosterone receptor; testicular feminization; spinal and bulbar | 2.0 |
| 134975 | R50333 | Hs.92186 | Leman coiled-coil protein | 2.6 |
| 135011 | AB037835 | Hs.92991 | KIAA1414 protein | 1.4 |
| 135022 | NM_000408 | Hs.93201 | glycerol-3-phosphate dehydrogenase 2 (mi | 1.6 |
| 135032 | AW301984 | Hs.173685 | hypothetical protein FLJ12619 | 1.4 |
| 135077 | AW503733 | Hs.9414 | KIAA1488 protein | 1.8 |
| 135083 | AB036063 | Hs.94262 | p53-inducible ribonucleotide reductase s | 2.5 |
| 135095 | AF027219 | Hs.9443 | zinc finger protein 202 | 1.5 |
| 135096 | AA081258 | Hs.132390 | zinc finger protein 36 (KOX 18) | 2.1 |
| 135153 | AI093155 | Hs.95420 | JM27 protein | 4.4 |
| 135181 | BE250865 | Hs.279529 | px19-like protein | 14.9 |
| 135199 | AA477514 | Hs.96247 | translin-associated factor X | 1.3 |
| 135207 | N26427 | Hs.9634 | ESTs, Highly similar to C10_HUMAN PUTATI | 1.7 |
| 135214 | T78802 | Hs.96560 | hypothetical protein FLJ11656 | 6.1 |
| 135243 | BE463721 | Hs.97101 | putative G protein-coupled receptor | 2.7 |
| 135245 | AI028767 | Hs.262603 | ESTs | 12.2 |
| 135257 | AW291023 | Hs.97255 | ESTs, Weakly similar to A46010 X-linked | 7.6 |
| 135263 | AI088775 | Hs.55498 | geranylgeranyl diphosphate synthase 1 | 1.8 |
| 135274 | AA448460 | Hs.112017 | GE36 gene | 4.1 |
| 135294 | AA150320 | Hs.9800 | protein kinase Njmu-R1 | 1.2 |
| 135295 | AI090838 | Hs.98006 | ESTs | 4.8 |
| 135307 | AI743770 | Hs.98368 | ESTs, Weakly similar to KIAA0822 protein | 5.8 |
| 135321 | AI652069 | Hs.98614 | ribosome binding protein 1 (dog 180 kD ho | 12.3 |
| 135354 | AA456454 | Hs.183418 | cell division cycle 2-like 1 (PITSLRE pr | 5.7 |
| 135361 | AA373452 | Hs.167700 | Homo sapiens cDNA FLJ10174 fis, clone HE | 7.9 |
| 135389 | U05237 | Hs.99872 | fetal Alzheimer antigen | 1.9 |
| 135400 | X78592 | Hs.99915 | androgen receptor (dihydrotestosterone r | 13.9 |
| 302256 | AA857131 | Hs.171595 | HIV TAT specific factor 1 | 5.3 |
| 302276 | AW057736 | Hs.323910 | HER2 receptor tyrosine kinase (c-erb-b2, | 2.2 |
| 303135 | AW592789 | Hs.279474 | HSPC070 protein | 1.4 |
| 303686 | AK000714 | Hs.109441 | MSTP033 protein | 5.2 |
| 310085 | R43191 | Hs.101248 | Homo sapiens clone IMAGE: 32553, mRNA seq | 2.3 |
| 315518 | AA808229 | Hs.167771 | ESTs | 2.8 |
| 317781 | NM_007057 | Hs.42650 | ZW10 interactor | 2.0 |
| 320836 | AI268997 | Hs.197289 | rab3 GTPase-activating protein, non-cata | 5.5 |
| 321114 | AA902256 | Hs.78979 | Golgi apparatus protein 1 | 1.4 |
| 322221 | N24236 | Hs.179662 | nucleosome assembly protein 1-like 1 | 1.3 |
| 322474 | AF118083 | Hs.29494 | PRO1912 protein | 2.9 |
| 322556 | BE041451 | Hs.177507 | hypothetical protein | 1.6 |
| 323541 | AF292100 | Hs.104613 | RP42 homolog | 1.8 |
| 407827 | BE278431 | Hs.40323 | BUB3 (budding uninhibited by benzimidazo | 1.6 |
| 408196 | AL034548 | Hs.43627 | SRY (sex determining region Y)-box 22 | 6.1 |
| 408813 | AI580090 | Hs.48295 | RNA helicase family | 5.6 |
| 409176 | R73727 | Hs.101617 | ESTs, Weakly similar to T32527 hypotheti | 2.6 |
| 413670 | AB000115 | Hs.75470 | hypothetical protein, expressed in osteo | 2.4 |
| 414108 | AI267592 | Hs.75761 | SFRS protein kinase 1 | 1.5 |
| 414846 | AW304454 | Hs.77495 | UBX domain-containing 1 | 4.2 |
| 416980 | AA381133 | Hs.80684 | high-mobility group (nonhistone chromoso | 23.6 |
| 417378 | R57256 | Hs.82037 | TATA box binding protein (TBP)-associate | 5.8 |
| 418283 | S79895 | Hs.83942 | cathepsin K (pycnodysostosis) | 1.3 |
| 418467 | NM_006910 | Hs.85273 | retinoblastoma-binding protein 6 | 1.6 |
| 420269 | U72937 | Hs.96264 | alpha thalassemia/mental retardation syn | 2.3 |
| 420802 | U22376 | Hs.1334 | v-myb avian myeloblastosis viral oncogen | 1.6 |
| 421225 | AA463798 | Hs.102696 | MCT-1 protein | 3.5 |
| 421642 | AF172066 | Hs.106346 | retinoic acid repressible protein | 4.9 |
| 421828 | AW891965 | Hs.279789 | histone deacetylase 3 | 3.1 |
| 421983 | AI252640 | Hs.110364 | peptidylprolyl isomerase C (cyclophilin | 1.9 |
| 422052 | AA302744 | Hs.104518 | ESTs | 2.4 |
| 422055 | NM_014320 | Hs.111029 | putative heme-binding protein | 4.1 |
| 423750 | AF165883 | Hs.298229 | prefoldin 2 | 7.0 |
| 424001 | W67883 | Hs.137476 | patemally expressed 10 (PEG10; KIAA105 | 4.9 |
| 425182 | AF041259 | Hs.155040 | zinc finger protein 217 | 3.4 |
| 425284 | AF155568 | Hs.155489 | NS1-associated protein 1 | 2.1 |
| 426372 | BE304680 | Hs.169531 | DEAD/H (Asp-Glu-Ala-Asp/His) box polypep | 7.5 |
| 428049 | AW183765 | Hs.182238 | GW128 protein | 1.7 |
| 428477 | AW500533 | Hs.11482 | splicing factor, arginine/serine-rich 11 | 2.4 |
| 437562 | AB001636 | Hs.5683 | DEAD/H (Asp-Glu-Ala-Asp/His) box polypep | 3.8 |
| 438449 | AK001333 | Hs.6216 | Homo sapiens hepatocellular carcinoma-as | 5.6 |
| 441560 | F13386 | Hs.7888 | Homo sapiens clone 23736 mRNA sequence | 2.0 |
| 445580 | AF167572 | Hs.12912 | skb1 (S. pombe) homolog | 7.5 |
| 446999 | AA151520 | Hs.334822 | hypothetical protein MGC4485 | 2.2 |
| 447111 | AI017574 | Hs.17409 | cysteine-rich protein 1 (intestinal) | 2.8 |
| 447778 | BE620592 | Hs.71190 | ESTs, Weakly similar to S16506 hypotheti | 1.7 |
| 448873 | NM_003677 | Hs.22393 | density-regulated protein | 5.9 |
| 449687 | W68520 | Hs.331328 | intermediate filament protein syncoilin | 5.6 |
| 450701 | H39960 | Hs.288467 | Homo sapiens cDNA FLJ12280 fis, clone MA | 1.4 |
| 450703 | AA011202 | Hs.184771 | nuclear factor I/C (CCAAT-binding transc | 4.7 |
| 452461 | N78223 | Hs.108106 | transcription factor | 2.9 |
| 452511 | BE408178 | Hs.285165 | Homo sapiens cDNA FLJ20845 fis, clone AD | 12.1 |
| 453157 | AF077036 | Hs.31989 | DKFZP586G1722 protein | 4.7 |
| 453658 | BE541906 | Hs.87819 | Homo sapiens, clone MGC: 2492, mRNA, comp | 1.3 |
| 100833 | AF135168 | Hs.108802 | N-ethylmaleimide-sensitive factor | 3.2 |
| 102481 | U50360 | | gb: Human calcium, calmodulin-dependent p | 6.2 |
| 102827 | BE244588 | Hs.6456 | chaperonin containing TCP1, subunit 2 (b | 7.9 |
| 103549 | BE270465 | Hs.78793 | protein kinase C, zeta | 2.0 |
| 104331 | AB040450 | Hs.279862 | cdk inhibitor p21 binding protein | 5.3 |
| 110018 | AW579842 | Hs.104557 | hypothetical protein FLJ10697 | 2.0 |
| 115008 | AK001827 | Hs.87889 | helicase-moi | 5.7 |
| 119075 | M10905 | Hs.287820 | fibronectin 1 | 1.3 |
| 119615 | AL034423 | Hs.75875 | ubiquitin-conjugating enzyme E2 variant | 2.9 |
| 125006 | BE065136 | Hs.145696 | splicing factor (CC1.3) | 1.7 |
| 127609 | X80031 | Hs.530 | collagen, type IV, alpha 3 (Goodpasture | 2.4 |
| 129209 | R62676 | Hs.17820 | Rho-associated, coiled-coil containing p | 5.2 |
| 129917 | M30773 | Hs.278540 | protein phosphatase 3 (formerly 2B), reg | 4.5 |
| 130182 | BE267033 | Hs.192853 | ubiquitin-conjugating enzyme E2G 2 (homo | 11.0 |
| 130365 | W56119 | Hs.155103 | eukaryotic translation initiation factor | 3.3 |
| 131135 | NM_016569 | Hs.267182 | TBX3-iso protein | 1.3 |
| 131853 | AI681917 | Hs.3321 | ESTs, Highly similar to IRX1_HUMAN IROQU | 3.2 |
| 131881 | AW361018 | Hs.3383 | upstream regulatory element binding prot | 14.3 |
| 132726 | N52298 | Hs.55608 | hypothetical protein MGC955 | 3.0 |
| 135193 | X95525 | Hs.96103 | TATA box binding protein (TBP)-associate | 2.7 |
| 409487 | H19886 | | gb: yn57a05.r1 Soares adult brain N2b5H | 2.3 |
| 416040 | AW819158 | Hs.289044 | Homo sapiens cDNA FLJ12048 fis, clone HE | 7.4 |

Pkey: Unique Eos probeset identifier number
ExAccn: Exemplar Accession number, Genbank accession number
UnigeneID: Unigene number
Unigene Title: Unigene gene title
R1: Ratio of tumor to normal breast tissue

TABLE 4A
Table 4A shows the accession numbers for those pkeys lacking unigeneID's for Table 4. For
each probeset, we have listed the gene cluster number from which the oligonucleotides were
designed. Gene clusters were compiled using sequences derived from Genbank ESTs and
mRNAs. These sequences were clustered based on sequence similarity using Clustering and
Alignment Tools (DoubleTwist, Oakland California). The Genbank accession numbers for
sequences comprising each cluster are listed in the “Accession” column.

| | |
|---|---|
| Pkey: | Unique Eos probeset identifier number |
| CAT number: | Gene cluster number |
| Accession: | Genbank accession numbers |

| Pkey | CAT number | Accessions |
|---|---|---|
| 123615 | 30686_-15 | AA609170 |
| 123619 | 371681_1 | AA602964 AA609200 |
| 101445 | 1650_-5 | M21259 |
| 124385 | 656394_1 | AI267847 N27351 |
| 124417 | 1642364_1 | N34059 N46979 |
| 124482 | 1657509_1 | N53935 N53950 |
| 102481 | 31281_-28 | U50360 |
| 103349 | 11052_-2 | X89059 |
| 110856 | 19346_14 | AA992380 N33063 N21418 H79958 R21911 H79957 |
| | | 103797 109699_1 AA080912 AA075318 AA083403 AA076594 AA078992 AA084926 AA081881 AA113913 AA113892 |
| | | AA083821 AA134801 AA082953 AA070343 AA062835 AA075419 AA063293 AA071252 AA078900 AA062836 AW974305 |
| 120280 | 160212_1 | AA190577 AA181657 |
| 113248 | 328626_1 | T63857 AW971220 AA493469 T63699 |
| 120472 | 44573_2 | AI950087 N70208 R97040 N36809 AI308119 AW967677 N35320 AI251473 H59397 AW971573 R97278 W01059 AW967671 |
| | | AA908598 AA251875 AI820501 AI820532 W87891 T85904 U71456 T82391 BE328571 T75102 R34725 AA884922 BE328517 |
| | | AI219788 AA884444 N92578 F13493 AA927794 AI560251 AW874068 AL134043 AW235363 AA663345 AW008282 AA488964 |
| | | AA283144 AI890387 AI950344 AI741346 AI689062 AA282915 AW102898 AI872193 AI763273 AW173586 AW150329 AI653832 |
| | | AI762688 AA988777 AA488892 AI356394 AW103813 AI539642 AA642789 AA856975 AW505512 AI961530 AW629970 |
| | | BE612881 AW276997 AW513601 AW512843 AA044209 AW856538 AA180009 AA337499 AW961101 AA251669 AA251874 |
| | | AI819225 AW205862 AI683338 AI858509 AW276905 AI633006 AA972584 AA908741 AW072629 AW513996 AA293273 |
| | | AA969759 N75628 N22388 H84729 H60052 T92487 AI022058 AA780419 AA551005 W80701 AW613456 AI373032 AI564269 |
| | | F00531 H83488 W37181 W78802 R66056 AI002839 R67840 AA300207 AW959581 T63226 F04005 |
| 129019 | 44573_2 | AI950087 N70208 R97040 N36809 AI308119 AW967677 N35320 AI251473 H59397 AW971573 R97278 W01059 AW967671 |
| | | AA908598 AA251875 AI820501 AI820532 W87891 T85904 U71456 T82391 BE328571 T75102 R34725 AA884922 BE328517 |
| | | AI219788 AA884444 N92578 F13493 AA927794 AI560251 AW874068 AL134043 AW235363 AA663345 AW008282 AA488964 |
| | | AA283144 AI890387 AI950344 AI741346 AI689062 AA282915 AW102898 AI872193 AI763273 AW173586 AW150329 AI653832 |
| | | AI762688 AA988777 AA488892 AI356394 AW103813 AI539642 AA642789 AA856975 AW505512 A1961530 AW629970 |
| | | BE612881 AW276997 AW513601 AW512843 AA044209 AW856538 AA180009 AA337499 AW961101 AA251669 AA251874 |
| | | AI819225 AW205862 AI683338 AI858509 AW276905 AI633006 AA972584 AA908741 AW072629 AW513996 M293273 |
| | | AA969759 N75628 N22388 H84729 H60052 T92487 AI022058 AA780419 AA551005 W80701 AW613456 AI373032 AI564269 |
| | | F00531 H83488 W37181 W78802 R66056 AI002839 R67840 AA300207 AW959581 T63226 F04005 |
| 120695 | 9683_3 | AA976503 AI917802 AA953664 AA404613 AA428771 BE280542 AW194691 AI927301 AI740458 AI796100 AI935603 AW052210 |
| | | AA970201 AI633384 AA425910 AI017004 AI241295 AA402816 AA291468 |
| 122188 | 275673_1 | AA398838 AA435847 |
| 121581 | 283769_1 | AA416568 AA442889 AA417233 AA442223 |
| 122618 | 305217_1 | AA453641 AA454061 |
| 109026 | 150431_1 | AA157811 AA836869 |
| 123658 | genbank_AA609364 | AA609364 |
| 123811 | genbank_AA620586 | AA620586 |
| 125115 | genbank_T97341 | T97341 |
| 125147 | NOT_FOUND_entrez_W38150 | W38150 |
| 118737 | 382979_1 | AA199686 N73861 |
| 120274 | genbank_AA177051 | AA177051 |
| 113196 | genbank_T57317 | T57317 |
| 120504 | genbank_AA256837 | AA256837 |
| 120639 | genbank_AA286942 | AA286942 |
| 120809 | genbank_AA346495 | AA346495 |
| 113702 | genbank_T97307 | T97307 |
| 129680 | 23162_1 | U03749 NM_001275 J03483 J03915 AI214509 AW245744 AL046455 AA318960 AI741505 AA843875 AI829382 AI560122 |
| | | AI858999 D55958 AI684005 D53170 AA854091 AI025609 D53119 D54729 D55504 D55377 D55313 AW512244 AA846441 |
| | | AW043898 AI969102 AA405741 AI091983 AA788784 AA706586 AA854361 AW470949 AA843095 AA772028 AI148432 |
| | | AI038109 AA782478 AA910064 AI220384 AA781296 AA843881 AA854064 AA843125 AA843419 AA319036 AA319054 |
| | | AI273831 W32275 AI584185 C05724 AA789023 AI686818 D54392 AI022485 AA431410 AA854232 W39212 W15214 AA894441 |
| | | AI803081 AI167381 AW245389 AA319430 AA335156 AI042646 AA327030 AA725170 T27943 AA889304 AA976699 AI687001 |
| | | AI621107 AI865540 AA772107 C06286 AA319661 AA405992 |
| 101045 | entrez_J05614 | J05614 |
| 117247 | genbank | N21032 |
| 110501 | genbank | H55748 |
| 103392 | entrez_X94563 | X94563 |
| 105032 | genbank | AA127818 |
| 119513 | NOT_FOUND_entrez | W37933 |
| 105445 | genbank | AA252395 |
| 121514 | genbank | AA412112 |
| 121558 | genbank | AA412497 |
| 121911 | genbank | AA427950 |
| 123315 | 714071_1 | AA496369 AA496646 |
| 114911 | genbank | AA236672 |
| 409487 | 1134778_1 | H19886 AW402806 T10231 |

TABLE 5
FIG. 5 from BRCA 001 US

Table 5 shows genes upregulated in tumor tissue compared to normal breast tissue.

| | |
|---|---|
| Pkey: | Unique Eos probeset identifier number |
| ExAccn: | Exemplar Accession number, Genbank accession number |
| UnigeneID: | Unigene number |
| Unigene Title: | Unigene gene title |
| R1: | Ratio of tumor to normal breast tissue |

| Pkey | ExAccn | UnigeneID | UnigeneTitle | R1 |
|---|---|---|---|---|
| 100114 | X02308 | Hs.82962 | thymidylate synthetase | 2.9 |
| 100147 | D13666 | Hs.136348 | osteoblast specific factor 2 (fasciclin | 7.5 |
| 100154 | H60720 | Hs.81892 | KIAA0101 gene praduct | 9.2 |
| 100335 | AW247529 | Hs.6793 | platelet-activating factor acetylhydrola | 2.7 |
| 100666 | L05424 | Hs.169610 | CD44 antigen (homing function and Indian | 5.7 |
| 100667 | L05424 | Hs.169610 | CD44 antigen (homing function and Indian | 9 |
| 100668 | L05424 | Hs.169610 | CD44 antigen (homing function and Indian | 7.6 |
| 100678 | AW502935 | Hs.740 | PTK2 protein tyrosine kinase 2 | 53.2 |
| 100988 | AK000405 | Hs.76480 | ubiquitin-like 4 | 11.4 |
| 101031 | J05070 | Hs.151738 | matrix metalloproteinase 9 (gelatinase B | 8.2 |
| 101045 | J05614 | | gb:Human proliferating cell nuclear anti | 5 |
| 101332 | J04088 | Hs.156346 | topoisomerase (DNA) II alpha (170kD) | 3.4 |
| 101352 | AI494299 | Hs.16297 | COX17 (yeast) homolog, cytochrome c oxid | 6.3 |
| 101580 | NM_012151 | Hs.83363 | coagulation factor VIII-associated (intr | 5.7 |
| 101592 | AF064853 | Hs.91299 | guanine nucleotide binding protein ( | 5.6 |
| 101767 | M81057 | Hs.180884 | carboxypeptidase B1 (tissue) | 14.4 |
| 101806 | AA586894 | Hs.112408 | S100 calcium-binding protein A7 (psorias | 8.9 |
| 101810 | NM_000318 | Hs.180612 | peroxisomal membrane protein 3 (35kD, Ze | 3.2 |
| 101983 | AI904232 | Hs.75323 | prohibitin | 8.4 |
| 102107 | BE258602 | Hs.182366 | heatshock protein 75 | 1.4 |
| 102165 | BE313280 | Hs.159627 | death associated protein 3 | 4.6 |
| 102198 | AW950852 | Hs.74598 | polymerase (DNA directed), delta 2, regu | 4.3 |
| 102217 | AA829978 | Hs.301613 | JTV1 gene | 6.7 |
| 102220 | U24389 | Hs.65436 | lysosomal | 4.3 |
| 102302 | AA306342 | Hs.69171 | protein kinase C-like 2 | 2.7 |
| 102348 | U37519 | Hs.87539 | aldehyde dehydrogenase 3 family, member | 2 |
| 102374 | U33635 | Hs.90572 | PTK7 protein tyrosine kinase 7 | 6.2 |
| 102455 | U48705 | Hs.75562 | discoidin domain receptor family, member | 6.9 |
| 102568 | W81489 | Hs.223025 | RAB31, member RAS oncogene family | 5.3 |
| 102618 | AL037672 | Hs.81071 | extracellular matrix protein 1 | 5.8 |
| 102687 | NM_007019 | Hs.93002 | ubiquitin carrier protein E2-C | 4.3 |
| 102689 | U96132 | Hs.171280 | hydroxyacyl-Coenzyme A dehydrogenase, ty | 6 |
| 102704 | AU077058 | Hs.54089 | BRCA1 associated RING domain 1 | 1.9 |
| 102705 | T97490 | Hs.50002 | small inducible cytokine subfamily A (Cy | 2.3 |
| 102801 | BE252241 | Hs.38041 | pyridoxal (pyridoxine, vitamin 86) kinas | 6.4 |
| 102827 | BE244588 | Hs.6456 | chaperonin containing TCP1, subunit 2 (b | 5.6 |
| 103060 | NM_005940 | Hs.155324 | matrix metalloproteinase 11 (MMP11; stro | 4.5 |
| 103080 | AU077231 | Hs.82932 | cyclin D1 (PRAD1: parathyroid adenomatos | 3.1 |
| 103178 | AA205475 | Hs.275865 | ribosomal protein S18 | 9.9 |
| 103206 | X72755 | Hs.77367 | monokine induced by gamma interferon | 8.8 |
| 103238 | AI369285 | Hs.75189 | death-associated protein | 5.6 |
| 103547 | AI376722 | Hs.180062 | proteasome (prosome, macropain) subunit, | 9.7 |
| 103549 | BE270465 | Hs.78793 | protein kinase C, zeta | 7.9 |
| 103886 | AK001278 | Hs.105737 | hypothetical protein FLJ10416 similar to | 6.5 |
| 104325 | BE379766 | Hs.150675 | polymerase (RNA) II (DNA directed) polyp | 6.3 |
| 104827 | AW052006 | Hs.8551 | PRP4/STK/WD splicing factor | 10.9 |
| 104846 | AI250789 | Hs.32478 | ESTs | 5.6 |
| 104854 | AA041276 | Hs.154729 | 3-phosphoinositide dependent protein kin | 12.3 |
| 104867 | AA278898 | Hs.225979 | hypothetical protein similar to small G | 2 |
| 104896 | AW015318 | Hs.23165 | ESTs | 17.7 |
| 104909 | AW408164 | Hs.249184 | transcnption factor 19 (SC1) | 5 |
| 104916 | AW958157 | Hs.155489 | NS1-associated protein 1 | 1.7 |
| 104919 | AA026880 | Hs.25252 | prolactin receptor | 1.4 |
| 104974 | Y12059 | Hs.278675 | bromodomain-containing 4 | 1.4 |
| 104978 | AI199268 | Hs.19322 | Homo sapiens, Similar to RIKEN cDNA 2010 | 7.2 |
| 105012 | AF098158 | Hs.9329 | chromosome 20 open reading frame 1 | 3.3 |
| 105039 | AA907305 | Hs.36475 | ESTs | 2.5 |
| 105079 | AA151342 | Hs.12677 | CGI-147 protein | 9.5 |
| 105088 | H58589 | Hs.35156 | Homo sapiens cDNA FLJ11027 fis, clone PL | 2.2 |
| 105393 | AF167570 | Hs.256583 | interleukin enhancer binding factor 3,9 | 5.4 |
| 105547 | AA262640 | Hs.27445 | unknown | 9.3 |
| 105554 | BE616694 | Hs.288042 | hypothetical protein FLJ14299 | 1.4 |
| 105658 | AA985190 | Hs.246875 | hypothetical protein FLJ20059 | 9.4 |
| 105746 | AW151952 | Hs.46679 | hypothetical protein FLJ20739 | 1.5 |
| 105858 | AF151066 | Hs.281428 | hypothetical protein | 2.9 |
| 105930 | AF016371 | Hs.9880 | peptidyl prolyl isomerase H (cyclophilin | 5.2 |
| 108094 | AA533491 | Hs.23317 | hypothetical protein FLJ14681 | 6.8 |
| 106350 | AK001404 | Hs.194698 | cyclin B2 | 5.7 |
| 106359 | AW390282 | Hs.31130 | transmembrane 7 superfamily member 2 | 6.3 |
| 106610 | AA458882 | Hs.79732 | fibulin 1 | 7.9 |
| 106624 | NM_003595 | Hs.26350 | tyrosylprotein sulfotransferase 2 | 7.7 |
| 106713 | BE614802 | Hs.184352 | hypothetical protein FLJ12549 | 4.5 |
| 106829 | AW959893 | Hs.27099 | hypothetical protein FLJ23293 similar to | 16.2 |
| 106846 | AB037744 | Hs.34892 | KIAA1323 protein | 2.2 |
| 106873 | N49809 | Hs.11197 | Homo sapiens, clone IMAGE:3343149, mRNA, | 16.8 |
| 106973 | BE156256 | Hs.11923 | hypothetical protein | 6.6 |
| 107029 | AF264750 | Hs.288971 | myeloid/lymphoid or mixed-lineage leukem | 1.8 |
| 107197 | W15477 | Hs.64639 | glioma pathogenesis-related protein | 6.1 |
| 107859 | AW732573 | Hs.47584 | potassium voltage-gated channel, delayed | 8.4 |
| 107901 | L42612 | Hs.335952 | keratin 68 | 2.5 |
| 107922 | BE153855 | Hs.61460 | Ig superfamily receptor LNIR | 2.2 |
| 107974 | AW956103 | Hs.61712 | pyruvate dehydrogenase kinase, isoenzyme | 6.7 |
| 108274 | AF129535 | Hs.272027 | F-box only protein 5 | 7.1 |
| 108647 | BE546947 | Hs.44276 | homeo box C10 | 9.8 |
| 108695 | AB029000 | Hs.70823 | KIAA1077 protein | 7.2 |
| 108894 | AK001431 | Hs.5105 | hypothetical protein FLJ10569 | 4 |
| 109011 | AA156542 | Hs.72127 | ESTs | 1.4 |
| 109068 | AA164293 | Hs.72545 | ESTs | 2.9 |
| 109273 | AA375752 | Hs.82719 | Homo sapiens mRNA; cDNA DKFZp586F1822 (f | 2.9 |
| 109468 | NM_015310 | Hs.6763 | KIAA0942 protein | 3.2 |
| 110240 | AI668594 | Hs.176588 | ESTs, Weakly similar to CP4Y_HUMAN CYTOC | 4.2 |
| 110330 | AI288666 | Hs.16621 | DKFZP434I116 protein | 6.2 |
| 110501 | H55748 | | gb:yq94a01.sl Soares fetal liver spleen | 6.1 |
| 110742 | AW190338 | Hs.28029 | hypothetical protein MGC11256 | 7.6 |
| 110762 | BE044245 | Hs.30011 | hypothetical protein MGC2963 | 9.3 |
| 110856 | AA992380 | | gb:ot37g06.s1 Soares_testis_NHT Homo sap | 2.3 |
| 110958 | NM_005864 | Hs.24587 | signal transduction protein (SH3 contain | 6.7 |
| 111125 | N63823 | Hs.269115 | ESTs, Moderately similar to Z195_HUMAN Z | 3.6 |
| 111179 | AK000136 | Hs.10760 | asporin (LRR class 1) | 7.1 |
| 111239 | N90956 | Hs.17230 | hypothetical protein FLJ22087 | 7.9 |
| 111285 | AA778711 | Hs.4310 | eukaryotic translation initiation factor | 6.9 |
| 111392 | W46342 | Hs.325081 | Homo sapiens, clone IMAGE:3659680, mRNA, | 8.4 |
| 111937 | BE298665 | Hs.14846 | Homo sapiens mRNA; cDNA DKFZp564D016 (fr | 10.6 |
| 112244 | AB029000 | Hs.70823 | KIAA1077 protein | 14.6 |
| 112995 | AA737033 | Hs.7155 | ESTs, Moderately similar to 2115357A TYK | 5.6 |
| 113777 | BE266947 | Hs.10590 | zinc finger protein 313 | 13.4 |
| 113791 | AI269096 | Hs.135578 | chitobiase, di-N-acetyl- | 1.3 |
| 113811 | BE207480 | Hs.6994 | Homo sapiens cDNA: FLJ22044 fis, clone H | 3.1 |
| 113834 | T26483 | Hs.6059 | EGF-containing fibulin-like extracellula | 11.3 |
| 113868 | W57902 | Hs.90744 | proteasome (prosome, macropain) 26S subu | 2.7 |
| 113870 | AL079314 | Hs.16537 | hypothetical protein, similar to (U06944 | 6.1 |
| 113923 | AW953484 | Hs.3849 | hypothetical protein FLJ22041 similar to | 1.9 |
| 114275 | AW515443 | Hs.306117 | KIAA0306 protein | 15.8 |
| 114895 | AA236177 | Hs.76591 | KIAA0887 protein | 7.1 |
| 114965 | AI733881 | Hs.72472 | BMP-R1B | 2.3 |
| 115061 | AI751438 | Hs.41271 | Homo sapiens mRNA full length insert cDN | 11.8 |
| 115278 | AK002163 | Hs.301724 | hypothetical protein FLJ11301 | 1.5 |
| 115291 | BE545072 | Hs.122579 | hypothetical protein FLJ10461 | 6.2 |
| 115652 | BE093589 | Hs.38178 | hypothetical protein FLJ23468 | 10.6 |
| 115693 | AF231023 | Hs.55173 | cadherin, EGF LAG seven-pass G-type rece | 6.8 |
| 115941 | AI867451 | Hs.46679 | hypothetical protein FLJ20739 | 5.5 |
| 115968 | AB037753 | Hs.62767 | KIAA1332 protein | 9.8 |
| 116011 | AL359053 | Hs.57664 | Homo sapiens mRNA full length insert cDN | 2.4 |
| 116417 | AW499664 | Hs.12484 | Human clone 23826 mRNA sequence | 7.4 |
| 116470 | AI272141 | Hs.83484 | SRY (sex determining region Y)-box 4 | 2.1 |
| 116637 | AK001043 | Hs.92033 | integrin-linked kinase-associated serine | 2.7 |
| 117132 | AI393666 | Hs.42315 | p10-binding protein | 5.2 |
| 117881 | AF161470 | Hs.260622 | butyrate-induced transcript 1 | 5.7 |
| 118528 | AI949952 | Hs.49397 | ESTs | 7.4 |
| 119075 | M10905 | Hs.287820 | fibronectin 1 | 5.7 |
| 119265 | BE539706 | Hs.285363 | ESTs | 1.4 |
| 119349 | T65004 | Hs.163561 | ESTs | 8.4 |
| 119403 | AL117554 | Hs.119908 | nucleolar protein NOP5/NOP58 | 6.7 |
| 119789 | BE393948 | Hs.50915 | kallikrein 5 (KLK5; KLK-L2; stratum com | 9.2 |
| 120206 | H26735 | Hs.91668 | Homo sapiens clone PP1498 unknown mRNA | 45.7 |
| 120253 | AA131376 | Hs.326401 | fibroblast growth factor 12B | 38.9 |
| 120297 | AA191384 | Hs.104072 | ESTs, Weakly similar to Z195_HUMAN ZINC | 15.2 |
| 120325 | AA195651 | Hs.104106 | ESTs | 6.4 |
| 120327 | AK000292 | Hs.278732 | hypothetical protein FLJ20285 | 16.1 |
| 120349 | AW969481 | Hs.55189 | hypothetical protein | 16.8 |
| 120356 | AF000545 | Hs.296433 | putative purinergic receptor | 28.1 |
| 120371 | AA219305 | Hs.104196 | EST | 12.4 |
| 120383 | AL109963 | Hs.123122 | FSH primary response (LRPR1, rat) homol | 9.7 |
| 120386 | AW969665 | Hs.154848 | hypothetical protein DKFZp434D0127 | 32.6 |
| 120389 | AW967985 | Hs.325572 | ESTs, Moderately similar to ALU7_HUMAN A | 21.7 |
| 120396 | AA134006 | Hs.79306 | eukaryotic translation initiation factor | 12.5 |
| 120418 | AW966893 | Hs.26613 | Homo sapiens mRNA; cDNA DKFZp586F1323 (f | 11.4 |
| 120472 | AI950087 | | gb:wq05c02.x1 NCI_CGAP_Kid12 Homo sapien | 19.4 |
| 120484 | AA253170 | Hs.96473 | EST | 10.4 |
| 120570 | AA280679 | Hs.271445 | ESTs, Weakly similar to ALU1_HUMAN ALU S | 14.4 |
| 120582 | BE244830 | Hs.284228 | ZNF135-like protein | 10.2 |
| 120596 | AA282074 | Hs.237323 | N-acetylglucosamine-phosphate mutase | 7.5 |
| 120624 | AW407987 | Hs.173518 | M-phase phosphoprotein homolog | 52 |
| 120695 | AA976503 | | gb:oq30a04.s1 NCI_CGAP_GC4 Homo sapiens | 46.8 |
| 120713 | AW449855 | Hs.96557 | Homo sapiens cDNA FLJ12727 fis, clone NT | 5.9 |
| 120750 | AI191410 | Hs.96693 | ESTs, Moderately similar to 2109260A B c | 7 |
| 120774 | AI608909 | Hs.193985 | ESTs | 7.8 |
| 120807 | AA346385 | Hs.30002 | SH3-containing protein SH3GLB2; KIAA1848 | 6.8 |
| 120809 | AA346495 | | gb:EST52657 Fetal heart II Homo sapiens | 4.4 |
| 120984 | BE262951 | Hs.99052 | ESTs | 5.6 |
| 121081 | AA398721 | Hs.186749 | ESTs, Highly similar to I37550 mismatch | 5.4 |
| 121408 | AA406137 | Hs.98019 | EST | 6 |
| 121505 | AA494172 | Hs.194417 | ESTs | 13.1 |
| 121508 | AA402515 | Hs.97887 | ESTs | 28 |
| 121513 | AA416653 | Hs.181510 | ESTs | 6.2 |
| 121549 | AA412477 | Hs.98142 | EST | 7.4 |
| 121558 | AA412497 | | gb:zt95g12.s1 Soares_testis_NHT Homo sap | 2.8 |
| 121655 | AA421537 | Hs.178072 | Homo sapiens mRNA; cDNA DKFZp434B1023 (f | 7.8 |
| 121744 | AA398784 | Hs.97514 | ESTs | 7.1 |
| 121748 | BE536911 | Hs.234545 | hypothetical protein NUF2R | 19.5 |
| 121773 | AB033022 | Hs.158654 | KIAA1196 protein | 7.9 |
| 121832 | AW340797 | Hs.98434 | ESTs | 5.8 |
| 121839 | AA425691 | Hs.191606 | ESTs, Highly similar to KIAA1048 protein | 5 |
| 121882 | AA426376 | Hs.98459 | ESTs | 5 |
| 121911 | AA427950 | | gb:zw50f02.s1 Soares_total_fetus_Nb2HF8_{—} | 7.2 |
| 121999 | AA430211 | Hs.98668 | EST | 6.4 |
| 122013 | AA431085 | Hs.98706 | ESTs | 6.5 |
| 122036 | W92142 | Hs.271963 | ESTs, Weakly similar to ALU5_HUMAN ALU S | 13.1 |
| 122356 | AA443794 | Hs.98390 | ESTs | 7.3 |
| 122371 | AA868555 | Hs.178222 | ESTs | 5 |
| 122372 | AA446008 | Hs.336677 | EST | 7.6 |
| 122460 | AW418788 | Hs.99148 | ESTs, Weakly similar to S43569 R01H10.6 | 9.7 |
| 122490 | AA448349 | Hs.238151 | EST | 6.1 |
| 122492 | AA448417 | Hs.104990 | ESTs | 5.4 |
| 122510 | AA449232 | Hs.99195 | ESTs | 11.2 |
| 122530 | AW959741 | Hs.40368 | adaptor-related protein complex 1, sigma | 10.1 |
| 122572 | AA452601 | Hs.99287 | EST | 11 |
| 122607 | AA453518 | Hs.98023 | ESTs | 61.5 |
| 122614 | AA453630 | Hs.99339 | EST | 10.7 |
| 122616 | AA453638 | Hs.161873 | ESTs | 107.3 |
| 122618 | AA453641 | | gb:zx48e06.s1 Soares_testis_NHT Homo sap | 31.1 |
| 122622 | AA453987 | Hs.144802 | ESTs | 5.6 |
| 122717 | AA456859 | Hs.178358 | ESTs | 8.5 |
| 122829 | AW204530 | Hs.99500 | ESTs | 81.8 |
| 122838 | AA460584 | Hs.334386 | ESTs | 75.3 |
| 122856 | AI929374 | Hs.75367 | Src-like-adapter | 5.8 |
| 122868 | AF005216 | Hs.115541 | Janus kinase 2 (a protein tyrosine kinas | 5.3 |
| 122907 | AA470074 | Hs.169896 | ESTs | 11.5 |
| 123016 | AW338067 | Hs.323231 | Homo sapiens cDNA FLJ11946 fis, clone HE | 2.8 |
| 123034 | AL359571 | Hs.44054 | ninein (GSK3B interacting protein) | 8.7 |
| 123136 | AW451999 | Hs.194024 | ESTs | 5.1 |
| 123152 | AW601773 | Hs.270259 | ESTs | 5.2 |
| 123394 | AA731404 | Hs.105510 | ESTs | 3.6 |
| 123466 | AA599042 | Hs.112503 | EST | 7.4 |
| 123486 | BE019072 | Hs.334802 | Homo sapiens cDNA FLJ14680 fis, clone NT | 2.4 |
| 123615 | AA609170 | | gb:af12a12.s1 Soares_testis_NHT Homo sap | 7.8 |
| 123735 | NM_013241 | Hs.95231 | FH1/FH2 domain-containing protein | 10 |
| 123753 | AA609955 | Hs.234961 | Huntingtin interacting protein E | 30.6 |
| 124006 | AI147155 | Hs.270016 | ESTs | 8.1 |
| 124385 | AI267847 | | gb:aq49a10.x1 Stanley Frontal NB pool 2 | 57.1 |
| 124440 | AA532519 | Hs.129043 | Human DNA sequence from clone 989H11 on | 7.8 |
| 124656 | AW297702 | Hs.102915 | ESTs | 8.3 |
| 124683 | AA381661 | Hs.119878 | ESTs, Weakly similar to M3K9_HUMAN MITOG | 7.9 |
| 124735 | R22952 | Hs.268685 | ESTs | 11.3 |
| 124761 | AA374756 | Hs.93560 | Homo sapiens mRNA for KIAA1771 protein, | 9 |
| 124768 | AW368528 | Hs.100855 | ESTs | 8.1 |
| 124788 | R43543 | Hs.100912 | Homo sapiens cDNA: FLJ22726 fis, clone H | 5.1 |
| 124811 | R46068 | Hs.288912 | hypothetical protein FLJ22604 | 14.2 |
| 124812 | R47948 | Hs.188732 | ESTs | 7.9 |
| 124822 | AA418160 | Hs.86043 | Homo sapiens cDNA FLJ13558 fis, clone PL | 6.6 |
| 124860 | R65763 | Hs.101477 | EST | 23.9 |
| 124903 | AW296713 | Hs.221441 | ESTs | 32.4 |
| 124930 | AI076343 | Hs.173939 | ESTs, Weakly similar to ALUB_HUMAN !!!! | 22.8 |
| 124942 | R99978 | Hs.268892 | ESTs, Moderately similar to B34087 hypot | 6.1 |
| 125051 | T79956 | Hs.100588 | EST | 135.3 |
| 125056 | T81310 | Hs.100592 | ESTs | 5.4 |
| 125101 | AI472068 | Hs.286236 | KIAA1856 protein | 5.6 |
| 125115 | T97341 | | gb:ye57e05.s1 Soares fetal liver spleen | 9.6 |
| 125280 | AI123705 | Hs.106932 | ESTs | 8 |
| 127274 | AW966158 | Hs.58582 | Homo sapiens cDNA FLJ12789 fis, clone NT | 12.8 |
| 128528 | R39234 | Hs.251699 | ESTs, Weakly similar to IDN4-GGTR14 [H.s | 2.8 |
| 128670 | AA975486 | Hs.103441 | Homo sapiens, Similar to RIKEN cDNA 1700 | 7.1 |
| 128691 | W27939 | Hs.103834 | hypothetical protein MGC5576 | 7.7 |
| 128772 | BE302796 | Hs.105097 | thymidine kinase 1, soluble | 5.3 |
| 128781 | N71826 | Hs.105465 | small nuclear ribonucleoprotein polypept | 53.9 |
| 128797 | NM_002975 | Hs.105927 | stem cell growth factor, lymphocyte secr | 13.3 |
| 128868 | AA419008 | Hs.106730 | chromosome 22 open reading frame 3 | 3 |
| 128891 | F34856 | Hs.292457 | Homo sapiens, clone MGC:16362, mRNA, com | 13.3 |
| 128946 | Y13153 | Hs.107318 | kynurenine 3-monooxygenase (kynurenine 3 | 7.2 |
| 128975 | BE560779 | Hs.284233 | NICE-5 protein | 14 |
| 12899S | AI816224 | Hs.107747 | DKFZP566C243 protein | 1.9 |
| 129019 | AI950087 | | gb:wq05c02.x1 NCI_CGAP_Kid12 Homo sapien | 2.9 |
| 129076 | AW296806 | Hs.326234 | ESTs, Highly similar to T46422 hypotheti | 5 |
| 129088 | AA744610 | Hs.194431 | palladin | 17.1 |
| 129096 | AA463189 | Hs.288906 | WW Domain-Containing Gene | 20.9 |
| 129198 | N57532 | Hs.109315 | KIAA1415 protein | 5.8 |
| 129347 | BE614192 | Hs.279869 | melanoma-associated antigen recognised b | 7.6 |
| 129362 | U30246 | Hs.110736 | solute carrier family 12 (sodium/potassi | 6.7 |
| 129372 | NM_016039 | Hs.110803 | CGI-99 protein | 2 |
| 129404 | AI267700 | Hs.317584 | ESTs | 5 |
| 129482 | AA188185 | Hs.289043 | spindlin | 6.7 |
| 129559 | W01296 | Hs.11360 | hypothetical protein FLJ14784 | 7.5 |
| 129587 | H14718 | Hs.11506 | Human clone 23589 mRNA sequence | 6.8 |
| 129629 | AK000398 | Hs.11747 | hypothetical protein FLJ20391 | 3.8 |
| 129649 | AD000092 | Hs.16488 | calreticulin | 3.3 |
| 129680 | U03749 | | gb:Human chromogranin A (CHGA) gene, pro | 14.1 |
| 129689 | AW748482 | Hs.77873 | B7 homolog 3 | 2.6 |
| 129702 | AI304966 | Hs.12035 | ESTs, Weakly similar to I38022 hypotheti | 7.4 |
| 129720 | AA156214 | Hs.12152 | APMCF1 protein | 2 |
| 130010 | AA301116 | Hs.142838 | nucleolar phosphoprotein Nopp34 | 1.6 |
| 130097 | AL046962 | Hs.14845 | forkhead box O3A | 2.8 |
| 130135 | AA311426 | Hs.21635 | tubulin, gamma 1 | 6.1 |
| 130211 | NM_003358 | Hs.23703 | ESTs, Moderately similar to CEGT_HUMAN C | 1.6 |
| 130242 | X79201 | Hs.153221 | synovial sarcoma, translocated to X chro | 5.4 |
| 130359 | NM_013449 | Hs.277401 | bromodomain adjacent to zinc finger doma | 8.5 |
| 130365 | W56119 | Hs.155103 | eukalyotic translation initiation factor | 11 |
| 130448 | BE513202 | Hs.15589 | PPAR binding protein | 3.9 |
| 130455 | D90041 | Hs.155956 | N-acetyltransferase 1 (arylamine N-acety | 33.6 |
| 130471 | AL121438 | Hs.183706 | adducin I (alpha) | 2.7 |
| 130503 | BE208491 | Hs.295112 | KIAA0618 gene product | 16.1 |
| 130511 | L32137 | Hs.1584 | cartilage oligomeric matrix protein (pse | 6.1 |
| 130642 | U64675 | Hs.179825 | RAN binding protein 2-like 1 | 7.8 |
| 130553 | AF062649 | Hs.252587 | pituitary tumor-transforming 1 | 14.4 |
| 130556 | AI907018 | Hs.15977 | Empirically selected from AFFX single pr | 4.7 |
| 130567 | AA383092 | Hs.1608 | replication protein A3 (14kD) | 7.9 |
| 130574 | AF083208 | Hs.16178 | apoptosis antagonizing transcription fac | 1.2 |
| 130617 | M90516 | Hs.1674 | glutamine-fructose-6-phosphate transamin | 12.1 |
| 130667 | BE246961 | Hs.17639 | Homo sapiens ubiquitin protein ligase (U | 13.9 |
| 130693 | R68537 | Hs.17962 | ESTs | 2 |
| 130744 | H59696 | Hs.18747 | POP7 (processing of precursor, S. carevi | 3.1 |
| 130757 | AL036067 | Hs.18925 | protein x 0001 | 5.7 |
| 130880 | BE514434 | Hs.20830 | kinesin-like 2 | 2.1 |
| 130944 | BE382657 | Hs.21486 | signal transducer and activator of trans | 5.4 |
| 131046 | AA321649 | Hs.2248 | small inducible cytokine subfamily B (Cy | 7.4 |
| 131060 | AA194422 | Hs.22564 | myosin VI | 5.1 |
| 131099 | AL133353 | Hs.226581 | COX15 (yeast) homolog, cytochrome c oxid | 7 |
| 131135 | NM_016569 | Hs.267182 | TBX3-iso protein | 3.3 |
| 131185 | BE280074 | Hs.23960 | cyclin B1 | 5.8 |
| 131225 | H62087 | Hs.31659 | thyroid hormone receptor-associated prot | 7.5 |
| 131245 | AL080080 | Hs.24766 | thioredoxin domain-containing | 2.8 |
| 131283 | X80038 | Hs.339713 | Homo sapiens clone F19374 APO E-C2 gene | 1.3 |
| 131569 | AL389951 | Hs.271623 | nucleoporin 50kD | 5 |
| 131643 | AW410601 | Hs.30026 | HSPC182 protein | 2.9 |
| 131714 | AA642831 | Hs.31016 | putative DNA binding protein | 2.9 |
| 131722 | D13757 | Hs.311 | phosphoribosyl pyrophosphate amidotransf | 3.4 |
| 131760 | X76732 | Hs.3164 | nucleobindin 2 | 2.9 |
| 131793 | AW966127 | Hs.32246 | Homo sapiens cDNA FU14656 fis, clone NT | 7.9 |
| 131885 | BE502341 | Hs.3402 | ESTs | 13.7 |
| 131900 | AA099014 | Hs.231029 | Homo sapiens, clone MGC:15961, mRNA, com | 8.7 |
| 131905 | AA179298 | Hs.3439 | stomatin-like 2 | 11.3 |
| 131941 | BE252983 | Hs.35086 | ubiquitin specific protease 1 | 2.3 |
| 131971 | BE567100 | Hs.154938 | hypothetical protein MDS025 | 3.5 |
| 132180 | NM_004460 | Hs.418 | fibroblast activation protein, alpha | 14.7 |
| 132203 | NM_004782 | Hs.194714 | synaptosomal-associated protein, 29kD | 7.8 |
| 132273 | AA227710 | Hs.43658 | DKFZP586L151 protein | 10 |
| 132288 | N36110 | Hs.305971 | solute carrier family 2 (facilitated glu | 9.2 |
| 132294 | AB023191 | Hs.44131 | KIAA0974 protein | 2 |
| 132348 | AW067708 | Hs.170311 | heterogeneous nuclear ribonucleoprotein | 12.5 |
| 132370 | AW572805 | Hs.46645 | ESTs | 28.3 |
| 132384 | AA312135 | Hs.46967 | HSPCO34 protein | 6.1 |
| 132450 | AA100012 | Hs.48827 | hypothetical protein FLJ12085 | 8.6 |
| 132465 | AW169847 | Hs.49169 | KIAA1634 protein | 6.1 |
| 132532 | AA454132 | Hs.5080 | mitochondrial ribosomal protein L16 | 7.1 |
| 132574 | AW631437 | Hs.5184 | TH1 drosophila homolog | 14 |
| 132638 | AI796870 | Hs.64277 | DNA segment on chromosome X (unique) 992 | 12.4 |
| 132718 | NM_004600 | Hs.564 | Sjogren syndrome antigen A2 (60kD, ribon | 3.7 |
| 132726 | N52298 | Hs.55608 | hypothetical protein MGC955 | 14.3 |
| 132731 | AI189075 | Hs.301872 | hypothetical protein MGC4840 | 5.9 |
| 132744 | AA010233 | Hs.55921 | glutamyl-prolyl-tRNA synthetase | 6.4 |
| 132773 | AA459713 | Hs.295901 | KIAA0493 protein | 14.6 |
| 132798 | AI026701 | Hs.5716 | KIAA0310 gene product | 2.5 |
| 132810 | A6007944 | Hs.5737 | KIAA0475 gene product | 4.2 |
| 132833 | U78525 | Hs.57783 | eukaryotic translation initiation factor | 6.1 |
| 132842 | NM_016154 | Hs.279771 | Homo sapiens clone PP1596 unknown mRNA | 7.1 |
| 132851 | U09716 | Hs.287912 | lectin, mannose-binding, 1 | 6.1 |
| 132891 | BE267143 | Hs.59271 | U2(RNU2) small nuclear RNA auxiliary fac | 2.7 |
| 132941 | AI817165 | Hs.6120 | hypothetical protein FLJ13222 | 2.1 |
| 132972 | AA034365 | Hs.288924 | Homo sapiens cDNA FLJ11392 fis, clone HE | 3.5 |
| 132980 | AA040696 | Hs.62016 | ESTs | 1.3 |
| 132994 | AA112748 | Hs.279905 | clone HQ0310 PRO0310p1 | 17.1 |
| 133016 | AI439688 | Hs.6289 | hypothetical protein FLJ20886 | 4.4 |
| 133177 | X97795 | Hs.66718 | RAD54 (S.cerevisiae)-like | 4.4 |
| 133208 | AI801777 | Hs.6774 | ESTs | 5.5 |
| 133254 | AI567421 | Hs.273330 | Homo sapiens, clone IMAGE:3544662, mRNA, | 1.3 |
| 133266 | AI160873 | Hs.69233 | zinc finger protein | 16.1 |
| 133268 | AW956781 | Hs.293937 | ESTs, Weakly similar to FXD2_HUMAN FORKH | 12.2 |
| 133285 | M76477 | Hs.289082 | GM2 ganglioside activator protein | 10.4 |
| 133390 | AI950382 | Hs.72660 | phosphatidylserine receptor | 5.7 |
| 133391 | AW103364 | Hs.727 | inhibin, beta A (activin A, activin AB a | 25.5 |
| 133540 | AL037159 | Hs.74619 | proteasome (prosome, macropain) 26S subu | 1.7 |
| 133594 | AW160781 | Hs.172589 | nuclear phosphoprotein similar to S. cer | 2.6 |
| 133621 | NM_004893 | Hs.75258 | H2A histone family, member Y | 13.5 |
| 133720 | L27841 | Hs.75737 | pericentriolar material 1 | 6.7 |
| 133760 | BE271766 | Hs.181357 | laminin receptor 1 (67kD, ribosomal prot | 5.4 |
| 133784 | BE622743 | Hs.301064 | arfaptin 1 | 12.1 |
| 133791 | M34338 | Hs.76244 | spermidine synthase | 9.7 |
| 133797 | AL133921 | Hs.76272 | retinoblastoma-binding protein 2 | 1.3 |
| 133822 | D50525 | Hs.699 | peptidyiprolyl isomerase B (cyclophilin | 9.7 |
| 133850 | W29092 | Hs.7678 | cellular retinoic add-binding protein 1 | 4.2 |
| 133865 | AB011155 | Hs.170290 | discs, large (Drosophila) homolog 5 | 5 |
| 133881 | U30872 | Hs.77204 | centromere protein F (350/400kD, mitosin | 9.1 |
| 133924 | D86326 | Hs.325948 | vesicle docking protein p115 | 1.8 |
| 133959 | X81789 | Hs.77897 | splicing factor 3a, subunit 3, 60kD | 10.4 |
| 133989 | AL040328 | Hs.78202 | SWI/SNF related, matrix associated, acti | 2.6 |
| 133997 | AI824113 | Hs.78281 | regulator of G-protein signalling 12 | 13 |
| 134234 | BE300078 | Hs.80449 | Homo sapiens, clone IMAGE:3535294, mRNA, | 10.3 |
| 134348 | AW291946 | Hs.82065 | interleukin 6 signal transducer (gp130, | 6.7 |
| 134376 | X06560 | Hs.82396 | 2′,5′-oligoadenylate synthetase 1 (40-46 | 5.5 |
| 134379 | AW362124 | Hs.323193 | hypothetical protein MGC3222 | 5.8 |
| 134405 | AW067903 | Hs.82772 | collagen, type XI, alpha 1 | 72.9 |
| 134421 | AU077196 | Hs.82985 | collagen, type V alpha 2 | 6.7 |
| 134480 | NM_005000 | Hs.83916 | Empirically selected from AFFX single pr | 6.2 |
| 134516 | AK001571 | Hs.273357 | hypothetical protein FLJ10709 | 1.4 |
| 134529 | AW411479 | Hs.848 | FK506-binding protein 4 (59kD) | 2.8 |
| 134751 | AW630803 | Hs.89497 | lamin B1 | 6.1 |
| 134790 | BE002798 | Hs.287850 | integral membrane protein 1 | 1.2 |
| 134806 | AD001528 | Hs.89718 | spermine synthase | 2.6 |
| 134850 | AI701162 | Hs.90207 | hypothetical protein MGC11138 | 9.1 |
| 134859 | D26488 | Hs.90315 | KIAA0007 protein | 13.3 |
| 134971 | AI097346 | Hs.286049 | phosphoserine aminotransferase | 2 |
| 135181 | BE250865 | Hs.279529 | px19-like protein | 14.9 |
| 135207 | N26427 | Hs.9634 | ESTs, Highly similar to C10_HUMAN PUTATI | 1.7 |
| 135245 | AI028767 | Hs.262603 | ESTs | 12.2 |
| 135257 | AW291023 | Hs.97255 | ESTs, Weakly similar to A46010 X-linked | 7.6 |
| 135307 | AI743770 | Hs.98368 | ESTs, Weakly similar to KIAA0822 protein | 5.8 |
| 135321 | AI652069 | Hs.98614 | ribosome binding protein 1 (dog 180kD ho | 12.3 |
| 135354 | AA456454 | Hs.183418 | cell division cycle 2-like 1 (PITSLRE pr | 5.7 |
| 135400 | X78592 | Hs.99915 | androgen receptor (dihydrotestosterone r | 13.9 |
| 302276 | AW057736 | Hs.323910 | HER2 receptor tyrosine kinase (c | 5.3 |
| 317781 | NM_007057 | Hs.42650 | ZW10 interactor | 2.8 |
| 321114 | AA902256 | Hs.78979 | Golgi apparatus protein 1 | 5.5 |
| 322556 | BE041451 | Hs.177507 | hypothetical protein | 2.9 |
| 420802 | U22376 | Hs.1334 | v-myb avian rayeloblastosis viral oncogen | 2.3 |
| 424001 | W67883 | Hs.137476 | paternally expressed 10 (PEG10; KIAA105 | 7 |
| 425182 | AF041259 | Hs.155040 | zinc finger protein 217 | 2.3 |
| 446999 | AA151520 | Hs.334822 | hypothetical protein MGC4485 | 7.5 |
| 450701 | H39960 | Hs.288467 | Homo sapiens cDNA FLJ12280 fis, clone MA | 5.6 |
| 452461 | N78223 | Hs.108106 | transcription factor | 4.7 |
| 453157 | AF077036 | Hs.31989 | DKFZP586G1722 protein | 12.1 |

TABLE 5A
Table 5A shows the accession numbers for those pkeys lacking unigeneID's for Table 5. For
each probeset, we have listed the gene cluster number from which the oligonucleotides were
designed. Gene clusters were compiled using sequences derived from Genbank ESTs and
mRNAs. These sequences were clustered based on sequence similarity using Clustering and
Alignment Tools (DoubleTwist, Oakland California). The Genbank accession numbers for
sequences comprising each cluster are listed in the “Accession” column.

| | |
|---|---|
| Pkey: | Unique Eos probeset identifier number |
| CAT number: | Gene cluster number |
| Accession: | Genbank accession numbers |

| Pkey | CAT number | Accessions |
|---|---|---|
| 123615 | 30686_-15 | AA609170 |
| 124385 | 656394_1 | AI267847 N27351 |
| 110856 | 19346_14 | AA992380 N33063 N21418 H79958 R21911 H79957 |
| 120472 | 44573_2 | AI950087 N70208 R97040 N36609 AI308119 AW967677 N35320 A1251473 H59397 AW971573 R97278 W01059 AW967671 |
| | | AA908598 AA251875 AI820501 AI820532 W87891 T85904 U71456 T82391 BE328571 T75102 R34725 AA884922 BE328517 |
| | | AI219788 AA884444 N92578 F13493 AA927794 AI560251 AW874068 AL134043 AW235363 AA663345 AW008282 |
| | | AA488964 AA283144 AI890387 AI950344 AI741346 AI689062 AA282915 AW102898 AI872193 AI763273 AW173586 |
| | | AW150329 AI653832 AI762688 AA988777 AA488892 AI356394 AW103813 AI539642 AA642789 AA856975 AW505512 |
| | | AI961530 AW629970 BE612881 AW276997 AW513601 AW512843 AA044209 AW856538 AA180009 AA337499 AW961101 |
| | | AA251669 AA251874 AI819225 AW205862 AI683338 AI858509 AW276905 A1633006 AA972584 AA908741 AW072629 |
| | | AW513996 AA293273 AA969759 N75628 N22388 H84729 H60052 T92487 AI022058 AA780419 AA551005 W80701 |
| | | AW613456 AI373032 AI564269 F00531 H83488 W37181 W78802 R66056 AI002839 R67840 AA300207 AW959581 T63226 |
| | | F04005 |
| 129019 | 445732 | AI950087 N70208 R97040 N36809 AI308119 AW967677 N35320 AI251473 H59397 AW971573 R97278 W01059 AW967671 |
| | | AA908598 AA251875 AI820501 AI820532 W87891 T85904 U71456 T82391 6E328571 T75102 R34725 AA884922 BE328517 |
| | | AI219788 AA884444 N92578 F13493 AA927794 AI560251 AW874068 AL134043 AW235363 AA663345 AW008282 |
| | | AA488964 AA283144 AI890387 AI950344 AI741346 AI689062 AA282915 AW102898 AI872193 AI763273 AW173586 |
| | | AW150329 AI653832 AI762688 AA988777 AA488892 AI356394 AW103813 AI539642 AA642789 AA856975 AW505512 |
| | | AI961530 AW629970 BE612881 AW276997 AW513601 AW512843 AA044209 AW856538 AA180009 AA337499 AW961101 |
| | | AA251669 AA251874 AI819225 AW205862 AI683338 AI858509 AW276905 AI633006 AA972584 AA908741 AW072629 |
| | | AW513996 AA293273 AA969759 N75628 N22388 H84729 H60052 T92487 AI022058 AA780419 AA551005 W80701 |
| | | AW613456 AI373032 AI564269 F00531 H83488 W37181 W78802 R66056 AI002839 R67840 AA300207 AW959581 T63226 |
| | | F04005 |
| | | 120695 9683_3 AA976503 AI917802 AA953664 AA404613 AA428771 BE280542 AW194691 AI927301 AI740458 |
| | | AI796100 AI935603 AW052210 AA970201 AI633384 AA425910 AI017004 AI241295 AA402816 AA291468 |
| 122618 | 305217_1 | AA453641 AA454061 |
| 125115 | genbank_T97341 | T97341 |
| 120809 | genbank_AA346495 | AA346495 |
| 129680 | 23162_1 | U03749 NM_001275 J03483 J03915 AI214509 AW245744 AL046455 AA318960 AI741505 AA843875 AI829382 AI560122 |
| | | AI858999 D55958 AI684005 D53170 AA854091 AI025609 D53119 D54729 D55504 D55377 D55313 AW512244 AA846441 |
| | | AW043898 AI969102 AA405741 AI091983 AA788784 AA706586 AA854361 AW470949 AA843095 AA772028 AI148432 |
| | | AI038109 AA782478 AA910064 AI220384 AA781296 AA843881 AA854064 AA843125 AA843419 AA319036 AA319054 |
| | | AI273831 W32275 AI584185 C05724 AA789023 AI686818 D54392 AI022485 AA431410 AA854232 W39212 W15214 |
| | | AA894441 AI803081 AI167381 AW245389 AA319430 AA335156 AI042646 AA327030 AA725170 T27943 AA889304 |
| | | AA976699 AI687001 AI621107 AI865540 AA772107 C06286 AA319661 M405992 |
| 101045 | entrez_j05614 | J05614 |
| 110501 | genbank_H55748 | H55748 |
| 121558 | genbank_AA412497 | AA412497 |
| 121911 | genbank_AA427950 | AA427950 |

TABLE 6
FIG. 6 from BRCA 001 US

Table 6 shows genes upregulated in tumor tissue compared to normal breast tissue.

| | |
|---|---|
| Pkey: | Unique Eos probeset identifier number |
| ExAccn: | Exemplar Accession number, Genbank accession number |
| UnigeneID: | Unigene number |
| Unigene Title: | Unigene gene title |
| R1: | Ratio of tumor to normal breast tissue |

| Pkey | ExAccn | UnigeneID | UnigeneTitle | R1 |
|---|---|---|---|---|
| 100147 | D13666 | Hs.136348 | osteoblast specific factor 2 (fasciclin | 7.5 |
| 100678 | AW502935 | Hs.740 | PTK2 protein tyrosine kinase 2 | 53.2 |
| 101806 | AA586894 | Hs.112408 | S100 calcium-binding protein A7 (psorias | 8.9 |
| 102455 | U48705 | Hs.75562 | discoidin domain receptor family, member | 6.9 |
| 103206 | X72755 | Hs.77367 | monokine induced by gamma interferon | 8.8 |
| 105743 | BE246502 | Hs.9598 | sema domain, immunoglobulin domain (Ig), | 2.6 |
| 105746 | AW151952 | Hs.46679 | hypothetical protein FLJ20739 | 1.5 |
| 106373 | AW503807 | Hs.21907 | histone acetyltransferase | 1.8 |
| 110240 | AI668594 | Hs.176588 | ESTs, Weakly similar to CP4Y_HUMAN CYTOC | 4.2 |
| 119260 | AK001724 | Hs.102950 | coat protein gamma-cop | 3.2 |
| 120206 | H26735 | Hs.91668 | Homo sapiens clone PP1498 unknown mRNA | 45.7 |
| 120253 | AA131376 | Hs.326401 | fibroblast growth factor 12B | 38.9 |
| 120297 | AA191384 | Hs.104072 | ESTs, Weakly similar to Z195_HUMAN ZINC | 15.2 |
| 120624 | AW407987 | Hs.173518 | M-phase phosphoprotein homolog | 52 |
| 120695 | AA976503 | | gb:oq30a04.s1 NCI_CGAP_GC4 Homo sapiens | 46.8 |
| 120807 | AA346385 | Hs.30002 | SH3-containing protein SH3GLB2; KIAA1848 | 6.8 |
| 121508 | AA402515 | Hs.97887 | ESTs | 28 |
| 122607 | AA453518 | Hs.98023 | ESTs | 61.5 |
| 122616 | AA453638 | Hs.161873 | ESTs | 107.3 |
| 122618 | AA453641 | | gb:zx48e06.s1 Soares_testis_NHT Homo sap | 31.1 |
| 122829 | AW204530 | Hs.99500 | ESTs | 81.8 |
| 122838 | AA460584 | Hs.334386 | ESTs | 75.3 |
| 123753 | AA609955 | Hs.234961 | Huntingtin interacting protein E | 30.6 |
| 124385 | AI267847 | | gb:aq49a10.x1 Stanley Frontal NB pool 2 | 57.1 |
| 124860 | R65763 | Hs.101477 | EST | 23.9 |
| 124930 | AI076343 | Hs.173939 | ESTs, Weakly similar to ALUB_HUMAN !!!! | 22.8 |
| 125051 | T79956 | Hs.100588 | EST | 135.3 |
| 128781 | N71826 | Hs.105465 | small nuclear ribonucleoprotein polypept | 53.9 |
| 129096 | AA463189 | Hs.288906 | WW Domain-Containing Gene | 20.9 |
| 129347 | BE614192 | Hs.279869 | melanoma-associated antigen recognised b | 7.6 |
| 129689 | AW748482 | Hs.77873 | B7 homolog 3 | 2.6 |
| 130503 | BE208491 | Hs.295112 | KIAA0618 gene product | 16.1 |
| 130511 | L32137 | Hs.1584 | cartilage oligomeric matrix protein (pse | 6.1 |
| 131046 | AA321649 | Hs.2248 | small inducible cytokine subfamily B (Cy | 7.4 |
| 131643 | AW410601 | Hs.30026 | HSPC182 protein | 2.9 |
| 131925 | AF151048 | Hs.183180 | anaphase promoting complex subunit 11 (y | 2.7 |
| 132180 | NM_004460 | Hs.418 | fibroblast activation protein, alpha | 14.7 |
| 132370 | AW572805 | Hs.46645 | ESTs | 28.3 |
| 132994 | AA112748 | Hs.279905 | clone HQ0310 PRO0310p1 | 17.1 |
| 133016 | AI439688 | Hs.6289 | hypothetical protein FLJ20886 | 4.4 |
| 133266 | AI160873 | Hs.69233 | zinc finger protein | 16.1 |
| 133391 | AW103364 | Hs.727 | inhibin, beta A (activin A, activin AB a | 25.5 |
| 134169 | AI690916 | Hs.178137 | transducer of ERBB2, 1 | 1.2 |
| 134219 | NM_000402 | Hs.80206 | glucose-6-phosphate dehydrogenase | 1.9 |
| 134405 | AW067903 | Hs.82772 | collagen, type XI, alpha 1 | 72.9 |
| 134529 | AW411479 | Hs.848 | FK506-binding protein 4 (59kD) | 2.8 |
| 134975 | R50333 | Hs.92186 | Leman coiled-coil protein | 2.6 |
| 135181 | BE250865 | Hs.279529 | px19-like protein | 14.9 |
| 322556 | BE041451 | Hs.177507 | hypothetical protein | 2.9 |

TABLE 6A
Table 6A shows the accession numbers for those pkeys lacking unigeneID's for Table 6. For
each probeset, we have listed the gene cluster number from which the oligonucleotides were
designed. Gene clusters were compiled using sequences derived from Genbank ESTs and
mRNAs. These sequences were clustered based on sequence similarity using Clustering and
Alignment Tools (DoubleTwist, Oakland California). The Genbank accession numbers for
sequences comprising each cluster are listed in the “Accession” column.

| | |
|---|---|
| Pkey: | Unique Eos probeset identifier number |
| CAT number: | Gene cluster number |
| Accession: | Genbank accession numbers |

| Pkey | CAT number | Accessions |
|---|---|---|
| 124385 | 656394_1 | AI267847 N27351 |
| 120695 | 9683_3 | AA976503 AI917802 AA953664 AA404613 AA428771 BE280542 AW194691 AI927301 AI740458 AI796100 AI935603 |
| | | AW052210 AA970201 AI633384 AA425910 AI017004 AI241295 AA402816 AA291468 |
| 122618 | 305217_1 | AA453641 AA454061 |

TABLE 7
FIG. 7 from BRCA 001-1 US

Table 7 shows genes upregulated in tumor tissue compared to normal breast tissue. Open
reading frames in the sequences have been characterized as having a signal sequence (SS), a
transmembrane domain (TM) or other.

| | |
|---|---|
| Pkey: | Unique Eos probeset identifier number |
| ExAccn: | Exemplar Accession number, Genbank accession number |
| UnigeneID: | Unigene number |
| Unigene Title: | Unigene gene title |
| R1: | Ratio of tumor to normal breast tissue |
| ORF struct info: | Structural characterization of open reading frame for the sequence of the gene |

| Pkey | ExAccn | UnigeneID | UnigeneTitle | R1 | ORF struct info |
|---|---|---|---|---|---|
| 100113 | NM_001269 | Hs.84746 | chromosome condensation 1 | 2.3 | TM |
| 100114 | X02308 | Hs.82962 | thymidylate synthetase | 2.9 | other |
| 100131 | D12485 | Hs.11951 | ectonucleotide pyrophosphatase/phosphodi | 1.9 | other |
| 100146 | BE185499 | Hs.2471 | K1AA0020 gene product | 1.9 | TM |
| 100147 | D13666 | Hs.136348 | osteoblast specific factor 2 (fasciclin | 7.6 | other |
| 100154 | H60720 | Hs.81892 | KIAA0101 gene product | 9.2 | other |
| 100163 | W44671 | Hs.124 | gene predicted from cDNA with a complete | 1.6 | other |
| 100220 | AW015534 | Hs.217493 | annexin A2 | 2 | other |
| 100265 | D38521 | Hs.112396 | KIAA0077 protein | 1.5 | other |
| 100271 | BE160081 | Hs.256290 | S100 calcium-binding protein All (calgiz | 13.5 | other |
| 100275 | BE242802 | Hs.154797 | KIAA0090 protein | 5.1 | other |
| 100323 | D50920 | Hs.23106 | KIAA0130 gene product | 1.9 | TM |
| 100335 | AW247529 | Hs.6793 | platelet-activating factor acetylhydrola | 2.7 | other |
| 100364 | NM_004341 | Hs.154868 | carbamoyl-phosphate synthetase 2, aspart | 2 | other |
| 100372 | NM_014791 | Hs.184339 | KIAA0175 gene product | 2.6 | other |
| 100393 | D84145 | Hs.39913 | novel RGD-containing protein | 3.2 | other |
| 100400 | AW954324 | Hs.75790 | phosphatidylinositol glycan, class c | 1.5 | other |
| 100418 | D86978 | Hs.84790 | KIAA0225 protein | 2 | other |
| 100482 | M65028 | Hs.81361 | heterogeneous nuclear ribonucleoprotein | 2.9 | other |
| 100518 | NM_004415 | Hs.74316 | desmoplakin (DPI, DPII) | 1.9 | other |
| 100666 | L05424 | Hs.169610 | CD44 antigen (homing function and Indian | 5.7 | other |
| 100667 | L05424 | Hs.169610 | CD44 antigen (homing function and Indian | 9 | ? |
| 100668 | L05424 | Hs.169610 | CD44 antigen (homing function and Indian | 7.7 | other |
| 100678 | AW502935 | Hs.740 | PTK2 protein tyrosine kinase 2 | 53.2 | other |
| 100783 | AF078847 | Hs.191356 | general transcription factor IIH, polype | 6 | other |
| 100892 | BE245294 | Hs.180789 | S164 protein | 1.7 | ? |
| 100945 | AF002225 | Hs.180686 | ubiquitin protein ligase E3A (human papi | 1.5 | other |
| 100969 | AA157634 | Hs.79172 | solute carrier family 25 (mitochondrial | 6.3 | other |
| 100988 | AK000405 | Hs.76480 | ubiquitin-like 4 | 11.4 | ? |
| 100999 | H38765 | Hs.80706 | diaphorase (NADH/NADPH) (cytochrome b-5 | 1.6 | other |
| 101031 | J05070 | Hs.151738 | matrix metalloproteinase 9 (gelatinase B | 8.4 | other |
| 101045 | J05614 | | gb:Human proliferating cell nuclear anti | 5 | ? |
| 101077 | N99692 | Hs.75227 | Empirically selected from AFEX single pr | 2.6 | other |
| 101093 | L06419 | Hs.75093 | procollagen-lysine, 2-oxoglutarate 5-die | 1.4 | ? |
| 101186 | AA020956 | Hs.179881 | core-binding factor, beta subunit | 2 | TM |
| 101216 | AA284166 | Hs.84113 | cyclin-dependent kinase inhibitor 3 (COK | 1.8 | other |
| 101228 | AA333387 | Hs.82916 | chaperonin containing TCP1, subunit6A ( | 1.7 | TM |
| 101247 | AA132666 | Hs.78802 | glycogen synthase kinase 3 beta | 1.9 | other |
| 101249 | L18964 | Hs.1904 | protein kinase C, iota | 1.5 | other |
| 101332 | J04088 | Hs.156346 | topoisomerase (DNA) II alpha (170kD) | 5.3 | other |
| 101352 | AI494299 | Hs.16297 | COX17 (yeast) homolog, cytochrome c oxid | 4.2 | other |
| 101396 | BE267931 | Hs.78996 | proliferating cell nuclear antigen | 1.9 | TM |
| 101445 | M21259 | | gb:Human Alu repeats in the region 5′ to | 1.6 | TM |
| 101470 | NM_000546 | Hs.1846 | tumor protein p53 (Li-Fraumeni syndrome) | 2.5 | other |
| 101478 | NM_002890 | Hs.758 | RAS p21 protein activator (GTPase activa | 5.5 | other |
| 101483 | M24486 | Hs.76768 | procollagen-proline, 2-oxoglutarate 4-di | 2.1 | other |
| 101540 | J04977 | Hs.84981 | X-ray repair complementing defective rep | 1.6 | other |
| 101573 | AW248421 | Hs.250758 | proteasome (prosome, macropain) 26S subu | 5.7 | other |
| 101580 | NM_012151 | Hs.83363 | coagulation factor VIII-associated (intr | 1.8 | other |
| 101592 | AF064853 | Hs.91299 | guanine nucleotide binding protein (G pr | 5.6 | ? |
| 101621 | BE391804 | Hs.62661 | guanylate binding protein 1, interferon- | 2.4 | other |
| 101702 | AW504089 | Hs.179574 | protein phosphatase 2 (formerly 2A), reg | 1.3 | other |
| 101734 | M74099 | Hs.147049 | cut (Drosophila)-like 1 (CCAAT displacem | 2.1 | ? |
| 101759 | M80244 | Hs.184601 | solute cariier family 7 (cationic amino | 5 | TM |
| 101767 | M81057 | Hs.180884 | carboxypeptidase B1 (tissue) | 14.4 | SS, |
| 101782 | AA306495 | Hs.1869 | phosphoglucomutase 1 | 5.2 | other |
| 101805 | AW409747 | Hs.75612 | stress-induced-phosphoprotein 1 (Hsp70/H | 8.6 | other |
| 101806 | AA586894 | Hs.112408 | S100 calcium-binding protein A7 (psorias | 8.9 | SS, TM |
| 101810 | NM_000318 | Hs.180612 | peroxisomal membrane protein 3 (35kD, Ze | 3.2 | TM |
| 101879 | AA176374 | Hs.243886 | nuclear autoantigenic sperm protein (his | 1.6 | other |
| 101911 | AA441787 | Hs.1 19689 | glycoprotein hormones, alpha polypeptide | 31.3 | ? |
| 101920 | AF182645 | Hs.8024 | IK cytokine, down-regulator of HLA II | 1.8 | other |
| 101973 | U41514 | Hs.80120 | UDP-N-acetyl-alpha-D-galactosamine:polyp | 2.4 | other |
| 102009 | BE245149 | Hs.82643 | protein tyrosine kinase 9 | 1.3 | other |
| 102036 | BE250127 | Hs.82906 | CDC20 (coil division cycle 20, S. cerevi | 2 | ? |
| 102083 | T35901 | Hs.75117 | interleukin enhancer binding factor 2,4 | 1.6 | other |
| 102107 | BE258602 | Hs.182366 | heat shock protein 75 | 1.4 | other |
| 102123 | NM_001809 | Hs.1594 | centromere protein A (17kD) | 1.8 | other |
| 102165 | BE313280 | Hs.159627 | death associated protein 3 | 4.6 | ? |
| 102198 | AW950852 | Hs.74598 | polymerase (DNA directed), delta 2, regu | 4.4 | ? |
| 102217 | AA829978 | Hs.301613 | JTV1 gene | 6.7 | other |
| 102220 | U24389 | Hs.65436 | lysosomal | 4.4 | TM |
| 102234 | AW163390 | Hs.278554 | heterochromatin-like protein 1 | 1.9 | TM |
| 102260 | AL039104 | Hs.159557 | karyopherin alpha 2 (RAG cohort 1, impor | 4.4 | other |
| 102302 | AA306342 | Hs.69171 | protein kinase C-like 2 | 2.7 | ? |
| 102330 | BE298063 | Hs.77254 | chromobox homolog 1 (Drosophila HP1 beta | 1.5 | other |
| 102339 | BE378432 | Hs.95577 | cyclin-dependent kinase 4 | 2.3 | TM |
| 102348 | U37519 | Hs.87539 | aldehyde dehydrogenase 3 family, member | 2 | TM |
| 102349 | AU077055 | Hs.289107 | baculoviral IAP repeat-containing 2 | 3.2 | other |
| 102369 | U39840 | Hs.299867 | hepatocyte nuclear factor 3, alpha | 2 | other |
| 102374 | U33635 | Hs.90572 | PTK7 protein tyrosine kinase 7 | 6.2 | other |
| 102391 | AA296874 | Hs.77494 | deoxyguanosine kinase | 1.5 | TM |
| 102455 | U48705 | Hs.75562 | discoidin domain receptor family, member | 7 | other |
| 102465 | NM_001359 | Hs.81548 | 2,4-dienoyl CoA reductase 1, mitochondri | 1.8 | SS, |
| 102488 | U50939 | Hs.61828 | amyloid beta precursor protein-binding p | 1.5 | ? |
| 102489 | AL080116 | Hs.74420 | origin recognition complex, subunit 3 (y | 3.3 | other |
| 102494 | AI188137 | Hs.75193 | COP9 homolog | 2.1 | other |
| 102501 | AF217197 | Hs.74562 | siah binding protein 1; FBP interacting | 3.2 | other |
| 102522 | BE250944 | Hs.183556 | solute carrier family 1 (neutral amino a | 2.8 | ? |
| 102532 | AF040253 | Hs.70186 | suppressor of Ty (S.cerevisiae) 5 homolo | 5.7 | ? |
| 102554 | U59423 | Hs.79067 | MAD (mothers against decapentaplegic, Dr | 2.3 | other |
| 102568 | W81489 | Hs.223025 | RAB31, member RAS oncogene family | 5.3 | other |
| 102580 | U60808 | Hs.152981 | CDP-diacylglycerol synthase (phosphatida | 2.1 | other |
| 102581 | AU077228 | Hs.77256 | enhancer of zeste (Drosophila) homolog 2 | 1.6 | ? |
| 102582 | U61232 | Hs.32675 | tubulin-specific chaperone e | 2.1 | other |
| 102617 | AW161453 | Hs.196767 | COP9 (constitutive photomorphogenic, Ara | 1.8 | other |
| 102618 | AL037672 | Hs.81071 | extracellular matrix protein 1 | 5.8 | other |
| 102627 | AL021918 | Hs.158174 | zinc finger protein 184 (Kruppel-like) | 1.3 | other |
| 102663 | NM_002270 | Hs.168075 | karyopherin (importin) beta 2 | 1.8 | TM |
| 102676 | BE262989 | Hs.12045 | putative protein | 2.3 | other |
| 102687 | NM_007019 | Hs.93002 | ubiquitin canier protein E2-C | 4.4 | ? |
| 102689 | U96132 | Hs.171280 | hydroxyacyl-Coenzyme A dehydrogenase, ty | 6 | ? |
| 102696 | BE540274 | Hs.239 | forkhead box M1 | 4.2 | other |
| 102704 | AU077058 | Hs.54089 | BRCA1 associated RING domain 1 | 1.9 | other |
| 102705 | T97490 | Hs.50002 | small inducible cytokine subfamily A (Cy | 2.3 | SS, TM |
| 102750 | AB014460 | Hs.66196 | nth (E.coli endonuclease III)-like 1 | 1.2 | TM |
| 102801 | BE252241 | Hs.38041 | pyridoxal (pyridoxine, vitamin B6) kinas | 6.5 | other |
| 102812 | U90549 | Hs.236774 | high-mobility group (nonhistone chromoso | 1.6 | other |
| 102827 | BE244588 | Hs.6456 | chaperonin containing TCP1, subunit 2 (b | 5.6 | TM |
| 102844 | AV653790 | Hs.324275 | WW domain-containing protein 1 | 1.3 | TM |
| 102868 | X02419 | Hs.77274 | plasminogen activator, urokinase | 4.4 | other |
| 102925 | BE440142 | Hs.2943 | signal recognition particle 19kD | 1.9 | other |
| 102935 | BE561850 | Hs.80506 | small nuclear ribonucleoprotein polypept | 2.4 | ? |
| 102968 | AU076611 | Hs.154672 | methylene tetrahydrofolate dehydrogenase | 2.7 | other |
| 102983 | BE387202 | Hs.118638 | non-metastatic cells 1, protein (NM23A) | 3.1 | other |
| 102985 | U95742 | Hs.2707 | G1 to S phase transition 1 | 5.2 | ? |
| 103023 | AW500470 | Hs.117950 | multifunctional polypeptide similar to S | 1.6 | other |
| 103038 | AA926960 | Hs.334883 | CDC28 protein kinase 1 | 2.5 | TM |
| 103060 | NM_005940 | Hs.155324 | matrix metalloproteinase 11 (MMP11; stro | 4.5 | other |
| 103080 | AU077231 | Hs.82932 | cyclin D1 (PRAD1: parathyroid adenomatos | 3.1 | other |
| 103089 | D31152 | Hs.179729 | collagen, type X, alpha 1 (Schmid metaph | 2.4 | other |
| 103177 | BE244377 | Hs.48876 | famesyl-diphosphate famesyltransferase | 3.5 | other |
| 103178 | AA205475 | Hs.275865 | ribosomal protein S18 | 9.9 | ? |
| 103179 | NM_001777 | Hs.82685 | CD47 antigen (Rh-related antigen, integr | 1.3 | other |
| 103181 | X69636 | Hs.334731 | Homo sapiens, clone IMAGE:3448306, mRNA, | 2 | other |
| 103185 | NM_006825 | Hs.74368 | transmembrane protein (63kD), endoplasmi | 1.6 | other |
| 103191 | AA401039 | Hs.2903 | protein phosphatase 4 (formerly X), cata | 2.5 | other |
| 103193 | NM_004766 | Hs.75724 | coatomer protein complex, subunit beta 2 | 2.2 | TM |
| 103194 | NM_004939 | Hs.78580 | DEAD/H (Asp-Glu-Ala-Asp/His) box polypep | 6.3 | TM |
| 103206 | X72755 | Hs.77367 | monokine induced by gamma interferon | 8.8 | TM |
| 103223 | BE275607 | Hs.1708 | chaperonin containing TCP1,subunit 3 (g | 3 | other |
| 103232 | X75962 | Hs.129780 | tumor necrosis factor receptor superfami | 1.8 | other |
| 103238 | AI369285 | Hs.75189 | death-associated protein | 5.6 | TM |
| 103297 | NM_001545 | Hs.9078 | immature colon carcinoma transcript 1 | 1.9 | ? |
| 103330 | AI803447 | Hs.77496 | small nuclear ribonucleoprotein polypept | 2.5 | other |
| 103349 | X89059 | | gb:H.sapiens mRNA for unknown protein ex | 1.6 | other |
| 103376 | AL036166 | Hs.323378 | coated vesicle membrane protein | 1.8 | other |
| 103391 | X94453 | Hs.114366 | pyrroline-5-carboxylate synthetase (glut | 2.3 | other |
| 103392 | X94563 | | gb:H.sapiens dbi/acbp gene exon 1 & 2. | 4 | TM |
| 103430 | BE564090 | Hs.20716 | translocase of inner mitochondrial membr | 1.3 | other |
| 103491 | AF264750 | Hs.288971 | myeloid/lymphoid or mixed-lineage leukem | 5.7 | ? |
| 103505 | AL031224 | Hs.33102 | transcription factor AP-2 beta (activati | 5.1 | other |
| 103547 | A1376722 | Hs.180062 | proteasome (prosome, macropain) subunit, | 9.7 | ? |
| 103588 | NM_006218 | Hs.85701 | phosphoinositide-3-kinase, catalytic, al | 2 | other |
| 103613 | NM_000346 | Hs.2316 | SRY (sex determining region Y)-box 9 (ca | 1.3 | ? |
| 103621 | BE379766 | Hs.150675 | polymerase (RNA) II (DNA directed) polyp | 2 | other |
| 103622 | AA609685 | Hs.278672 | membrane component, chromosome 11, surfa | 2.3 | TM |
| 103727 | AI878883 | Hs.296381 | growth factor receptor-bound protein 2 | 1.3 | other |
| 103754 | AI015709 | Hs.172089 | Homo sapiens mRNA; cDNA DKFZp586I2022 (f | 1.3 | other |
| 103780 | AA094752 | Hs.169992 | hypothetical 43.2 Kd protein | 7.6 | ? |
| 103795 | H26531 | Hs.7367 | Homo sapiens BTB domain protein (BDPL) m | 1.3 | SS, TM |
| 103797 | AA080912 | | gb:zn04d03.r1 Stratagene hNT neuron (937 | 1.6 | other |
| 103813 | AI042582 | Hs.181271 | CGI-120 protein | 1.6 | other |
| 103855 | W02363 | Hs.302267 | hypothetical protein FLJ10330 | 1.6 | other |
| 103886 | AK001278 | Hs.105737 | hypothetical protein FLJ10416 similar to | 6.6 | TM |
| 104052 | NM_002407 | Hs.97644 | mammaglobin 2 | 2.9 | other |
| 104079 | AA251242 | Hs.103238 | ESTs | 1.4 | other |
| 104174 | AA478984 | Hs.6451 | PRO0659 protein | 5.6 | TM |
| 104227 | AB002343 | Hs.98938 | protocadherin alpha 9 | 1.6 | other |
| 104275 | AI751970 | Hs.101067 | GCN5 (general control of amino-acid synt | 5.4 | other |
| 104325 | BE379766 | Hs.150675 | polymerase (RNA) II (DNA directed) polyp | 6.4 | other |
| 104370 | AA324597 | Hs.21851 | Homo sapiens CDNA FLJ12900 fis, clone NT | 1.6 | other |
| 104423 | R83113 | Hs.1432 | protein kinase C substrate 80K-H | 5.2 | other |
| 104482 | AB037762 | Hs.44268 | myalin gene expression factor 2 | 1.2 | other |
| 104667 | AI239923 | Hs.30098 | ESTs | 1.4 | other |
| 104757 | AI694413 | Hs.332549 | olfactory receptor, family 2, subfamily | 2.4 | other |
| 104804 | AI858702 | Hs.31803 | ESTs, Weakly similar to N-WASP [H.sapien | 1.4 | other |
| 104806 | AB023175 | Hs.22982 | KIAA0958 protein | 2.4 | other |
| 104827 | AW052006 | Hs.8551 | PRP4/STK/WD splicing factor | 10.9 | other |
| 104846 | AI250789 | Hs.32478 | ESTs | 5.7 | other |
| 104854 | AA041276 | Hs.154729 | 3-phosphoinositide dependent protein kin | 12.3 | ? |
| 104867 | AA278898 | Hs.225979 | hypothetical protein similar to small G | 2.1 | other |
| 104871 | T78044 | Hs.28893 | Homo sapiens mRNA; cDNA DKFZp564O2364 (f | 1.4 | other |
| 104896 | AW015318 | Hs.23165 | ESTs | 17.7 | other |
| 104909 | AW408164 | Hs.249184 | transcription factor 19 (SC1) | 5.1 | TM |
| 104916 | AW958157 | Hs.155489 | NS1-associated protein 1 | 1.8 | other |
| 104919 | AA026880 | Hs.25252 | prolactin receptor | 1.5 | other |
| 104930 | AF043467 | Hs.32893 | neurexophilin 2 | 2.3 | other |
| 104973 | NM_015310 | Hs.6763 | KIAA0942 protein | 5.1 | other |
| 104974 | Y12059 | Hs.278675 | bromodomain-containing 4 | 1.5 | other |
| 104975 | AL136877 | Hs.50758 | SMC4 (structural maintenance of chromoso | 2.4 | other |
| 104978 | AI199268 | Hs.19322 | Homo sapiens, Similar to RIKEN cDNA 2010 | 7.3 | other |
| 104979 | AA937934 | Hs.321062 | ESTs | 1.3 | other |
| 104994 | AI499930 | Hs.334885 | mitochondrial GTP binding protein | 3.6 | ? |
| 105009 | BE379584 | Hs.34789 | dolichyl-diphosphooligosaccharide-protei | 5.6 | other |
| 105012 | AF098158 | Hs.9329 | chromosome 20 open reading frame 1 | 3.4 | other |
| 105028 | AI050715 | Hs.2331 | E2F transcription factor 5, p130-binding | 2.2 | other |
| 105041 | AB037716 | Hs.26204 | KIAA1295 protein | 2.2 | other |
| 105045 | BE242899 | Hs.129951 | speckle-type POZ protein | 3.9 | ? |
| 105079 | AA151342 | Hs.12677 | CGI-147 protein | 9.5 | TM |
| 105087 | AA147884 | Hs.9812 | Homo sapiens cDNA FLJ14388 fis, clone HE | 5.7 | other |
| 105088 | H58589 | Hs.35156 | Homo sapiens cDNA FLJ11027 fis, clone PL | 2.2 | other |
| 105095 | Z78407 | Hs.27023 | vesicle transport-related protein | 2.2 | other |
| 105110 | BE387350 | Hs.33122 | KIAA1160 protein | 1.6 | other |
| 105126 | AW975433 | Hs.36288 | ESTs | 6.4 | ? |
| 105127 | AA045648 | Hs.301957 | nudix (nucleoside diphosphate linked mol | 2.2 | other |
| 105141 | AA164687 | Hs.177576 | mannosyl (alpha-1,3-)-glycoprotein beta- | 2.8 | other |
| 105158 | AW976357 | Hs.234545 | hypothetical protein NUF2R | 2 | other |
| 105169 | BE245294 | Hs.180789 | S164 protein | 1.7 | other |
| 105186 | AA191512 | Hs.28005 | Homo sapiens cDNA FLJ11309 fis, clone PL | 4.9 | SS, TM |
| 105254 | AA071276 | Hs.19469 | KIAA0859 protein | 2 | TM |
| 105281 | AA263143 | Hs.24596 | RAD51-interacting protein | 2.9 | ? |
| 105288 | N99673 | Hs.3585 | ESTs, Weakly similar to AF126743 1 DNAJ | 1.9 | TM |
| 105302 | AA700122 | Hs.3355 | sentrin-specific protease | 8.2 | ? |
| 105331 | AW270037 | Hs.179507 | KIAA0779 protein | 1.8 | SS, |
| 105359 | NM_016015 | Hs.8054 | CGI-68 protein | 8.4 | other |
| 105366 | BE264645 | Hs.282093 | hypothetical protein FLJ21918 | 5.1 | other |
| 105373 | AW887701 | Hs.32356 | hypothetical protein FLJ20628 | 2.6 | other |
| 105374 | BE242803 | Hs.262823 | hypothetical protein FLJ10326 | 2.2 | TM |
| 105387 | AW592146 | Hs.108636 | membrane protein CH1 | 2.3 | SS, TM |
| 105393 | AF167570 | Hs.256583 | interleukin enhancer binding factor 3,9 | 5.5 | SS, |
| 105399 | BE386877 | Hs.334811 | Npw38-binding protein NpwBP | 1.6 | other |
| 105400 | AF198620 | Hs.65648 | RNA binding motif protein BA | 1.6 | other |
| 105445 | AA252395 | | gb:zs12g10.s1 NCLCGAP_GCB1 Homo sapiens | 5.1 | ? |
| 105507 | BE268348 | Hs.226318 | CCR4-NOT transcription complex, subunit | 1.6 | other |
| 105529 | AA13449 | Hs.32471 | hypothetical protein FLJ20364 | 1.3 | other |
| 105530 | AB023179 | Hs.9059 | KIAA0962 protein | 3.5 | other |
| 105547 | AA262640 | Hs.27445 | unknown | 9.3 | other |
| 105554 | BE616694 | Hs.288042 | hypothetical protein FLJ14299 | 1.4 | other |
| 105596 | AA579535 | Hs.18490 | hypothetical protein FLJ20452 | 10.9 | TM |
| 105597 | AF054284 | Hs.334826 | splicing factor 3b, subunit 1, 155kD | 2.9 | TM |
| 105608 | AI808201 | Hs.287863 | hypothetical protein FLJ12475 | 1.7 | ? |
| 105610 | AA280072 | Hs.99872 | fetal Alzheimer antigen | 1.4 | other |
| 105617 | AK000892 | Hs.4069 | glucocorticoid modulatory element bindin | 1.7 | TM |
| 105620 | AW302245 | Hs.181390 | casein kinase 1, gamma 2 | 5.6 | other |
| 105658 | AA985190 | Hs.246875 | hypothetical protein FLJ20059 | 9.4 | other |
| 105697 | AW499988 | Hs.27801 | zinc finger protein 278 | 2 | TM |
| 105708 | R26944 | Hs.180777 | Homo sapiens mRNA; cDNA DKFZp564M0264 (f | 1.7 | other |
| 105743 | BE246502 | Hs.9598 | sema domain, immunoglobulin domain (Ig), | 2.7 | other |
| 105746 | AW151952 | Hs.46679 | hypothetical protein FLJ20739 | 1.5 | ? |
| 105759 | AI123118 | Hs.15159 | chemokine-like factor, altematively spl | 1.3 | other |
| 105771 | AI267720 | Hs.153221 | synovial sarcoma, translocated to X chro | 1.6 | other |
| 105820 | AA741336 | Hs.152108 | transctiptional unit N143 | 2.2 | other |
| 105826 | AA478756 | Hs.194477 | E3 ubiquitin ligase SMURF2 | 1.3 | other |
| 105856 | AI262106 | Hs.12653 | ESTs | 2.4 | other |
| 105858 | AF151066 | Hs.281428 | hypothetical protein | 2.9 | other |
| 105875 | AK001708 | Hs.32271 | hypothetical protein FU10846 | 1.4 | other |
| 105930 | AF016371 | Hs.9880 | peptidyl prolyl isomerase H (cyclophilin | 5.3 | other |
| 106000 | AW194426 | Hs.20726 | ESTs | 1.7 | other |
| 106011 | AW081202 | Hs.12284 | Homo sapiens, done IMAGE:2989556, mRNA, | 2.8 | other |
| 106017 | AA477956 | Hs.26268 | ESTs | 1.4 | other |
| 106073 | AL157441 | Hs.17834 | downstream neighbor of SON | 1.4 | other |
| 106078 | AA130158 | Hs.19977 | ESTs, Moderately similar to ALU8_HUMAN A | 1.6 | ? |
| 106094 | AA533491 | Hs.23317 | hypothetical protein FLJ14681 | 6.9 | other |
| 108140 | AB006624 | Hs.14912 | KIAA0286 protein | 1.6 | other |
| 106271 | AA251393 | Hs.289052 | Homo sapiens, Similar to RIKEN cDNA 5430 | 10.8 | ? |
| 106288 | A6037742 | Hs.24336 | KIAA1321 protein | 1.3 | other |
| 106300 | Y10043 | Hs.19114 | high-mobility group (nonhistone chromoso | 3.7 | other |
| 106333 | AL043114 | Hs.22410 | ESTs, Weakly similarto A54849 collagen | 5.5 | SS, |
| 106350 | AK001404 | Hs.194698 | cyclin B2 | 5.8 | other |
| 106359 | AW390282 | Hs.31130 | transmembrane 7 superfamily member 2 | 6.4 | other |
| 106381 | AB040916 | Hs.24106 | KIAA1483 protein | 6.6 | other |
| 106389 | AW748420 | Hs.6236 | Homo sapiens cDNA: FLJ21487 fis, clone C | 2.2 | TM |
| 106457 | AF119256 | Hs.27801 | zinc finger protein 278 | 2.7 | other |
| 106470 | D63078 | Hs.186180 | Homo sapiens cDNA: FLJ23038 fis, clone L | 2.3 | other |
| 106586 | AA243837 | Hs.57787 | ESTs | 1.6 | other |
| 106589 | AK000933 | Hs.28661 | Homo sapiens cDNA FU10071 fis, done HE | 2.4 | ? |
| 106610 | AA458882 | Hs.79732 | fibulin 1 | 8 | SS, |
| 106624 | NM_003595 | Hs.26350 | tyrosylprotein sulfotransferase 2 | 7.8 | other |
| 106650 | AL049951 | Hs.22370 | Homo sapiens mRNA; cDNA DKFZp564O0122 (f | 1.8 | other |
| 106669 | AV657117 | Hs.184164 | ESTs, Moderately similar to S65657 alpha | 1.3 | TM |
| 106713 | BE614802 | Hs.184352 | hypothetical protein FLJ12549 | 4.6 | other |
| 106717 | AA600357 | Hs.239489 | TIA1 cytotoxic granule-associated RNA-bi | 1.3 | other |
| 106723 | BE388094 | Hs.21857 | ESTs | 1.6 | SS, |
| 106795 | AF174487 | Hs.293753 | Bcl-2-related ovarian killer protein-lik | 5.7 | other |
| 106829 | AW959893 | Hs.27099 | hypothetical protein FLJ23293 similar to | 16.2 | TM |
| 106831 | BE564871 | Hs.29463 | centrin, EF-hand protein, 3 (CDC31 yeast | 1.5 | other |
| 106846 | AB037744 | Hs.34892 | KIAA1323 protein | 2.2 | other |
| 106852 | AF151031 | Hs.300631 | hypothetical protein | 1.3 | other |
| 106873 | N49809 | Hs.11197 | Homo sapiens, clone IMAGE:3343149, mRNA, | 16.8 | other |
| 106886 | W79171 | Hs.9567 | GL002 protein | 1.5 | TM |
| 106906 | AA861271 | Hs.222024 | transcription factor BMAL2 | 2.2 | other |
| 106920 | AK001838 | Hs.296323 | serum/glucocorticoid regulated kinase | 3.4 | other |
| 106945 | AK000511 | Hs.6294 | hypothetical protein DKFZp434L1435 simil | 6.8 | ? |
| 106973 | BE156256 | Hs.11923 | hypothetical protein | 6.7 | other |
| 106978 | AW631480 | Hs.8688 | ESTs | 6.1 | SS, |
| 107004 | AA146872 | Hs.300700 | hypothetical protein FLJ20727 | 1.3 | other |
| 107029 | AF264750 | Hs.288971 | myaloid/lymphoid or mixed-lineage leukem | 1.8 | other |
| 107071 | AW385224 | Hs.35198 | ectonucleotide pyrophosphatase/phosphodi | 1.7 | other |
| 107113 | AK000733 | Hs.23900 | GTPase activating protein | 2.5 | other |
| 107125 | AK000512 | Hs.69388 | hypothetical protein FLJ20505 | 1.7 | other |
| 107136 | AV661958 | Hs.8207 | GK001 protein | 4.7 | other |
| 107146 | AK001455 | Hs.5198 | Down syndrome critical region gene 2 | 2 | other |
| 107151 | AW378065 | Hs.8687 | ESTs | 6.4 | TM |
| 107155 | AW391927 | Hs.7946 | KIAA1288 protein | 33.5 | other |
| 107174 | BE122762 | Hs.25338 | ESTs | 5.2 | ? |
| 107197 | W15477 | Hs.64639 | glioma pathogenesis-related protein | 6.1 | other |
| 107221 | AW888411 | Hs.81915 | leukemia-associated phosphoprotein p18 ( | 17.4 | other |
| 107243 | BE219716 | Hs.34727 | ESTs, Moderately similar to 138759 zinc | 7.4 | ? |
| 107246 | AW263124 | Hs.315111 | nuclear receptor co-repressor/HDAC3 comp | 1.8 | other |
| 107263 | D60341 | Hs.21198 | translocase of outer mitochondrial membr | 6.7 | other |
| 107265 | BE379594 | Hs.49136 | ESTs, Moderately similar to ALU7_HUMAN A | 2.5 | other |
| 107299 | BE277457 | Hs.30661 | hypothetical protein MGC4606 | 3.2 | TM |
| 107316 | T63174 | Hs.193700 | Homo sapiens mRNA; cDNA DKFZp586IO324 (f | 2 | TM |
| 107354 | NM_006299 | Hs.96448 | zinc finger protein 193 | 5 | ? |
| 107392 | AW299900 | Hs.267632 | TATA element modulatoly factor 1 | 1.2 | other |
| 107481 | AA307703 | Hs.279766 | kinesin family member 4A | 1.6 | other |
| 107529 | BE515065 | Hs.296585 | nucleolar protein (KKE/D repeat) | 3 | TM |
| 107554 | AA001386 | Hs.59844 | ESTs | 1.4 | other |
| 107681 | BE379594 | Hs.49136 | ESTs, Moderately similar to ALU7_HUMAN A | 2.3 | SS, TM |
| 107772 | AA018587 | Hs.303055 | ESTs, Weakly similar to ALU1_HUMAN ALU S | 2.2 | ? |
| 107859 | AW732573 | Hs.47584 | potassium voltage-gated channel, delayed | 8.4 | TM |
| 107901 | L42612 | Hs.335952 | keratin 66 | 2.5 | other |
| 107922 | BE153855 | Hs.61460 | Ig superfamily receptor LNIR | 2.3 | other |
| 107974 | AW956103 | Hs.61712 | pyruvate dehydrogenase kinase, isoenzyme | 6.8 | other |
| 108040 | AL121031 | Hs.159971 | SWI/SNF related, matrix associated, acti | 1.6 | other |
| 108230 | AA054224 | Hs.59847 | ESTs | 1.3 | other |
| 108274 | AF129535 | Hs.272027 | F-box only protein 5 | 7.2 | ? |
| 108296 | N31256 | Hs.161623 | ESTs | 2.6 | other |
| 108496 | AA083069 | Hs.339659 | ESTs | 3.6 | other |
| 108607 | BE300380 | Hs.69476 | Homo sapiens cDNA FLJ12758 fis, clone NT | 3.5 | other |
| 108621 | AA101809 | Hs.182685 | ESTs | 1.7 | other |
| 108634 | AW022410 | Hs.69507 | ESTs | 1.8 | SS, TM |
| 108647 | BE546947 | Hs.44276 | homeo box 010 | 9.8 | other |
| 108695 | AB029000 | Hs.70823 | KIAA1077 protein | 7.3 | other |
| 108740 | AI089575 | Hs.9071 | progesterone membrane binding protein | 2.8 | ? |
| 108828 | AK001693 | Hs.273344 | DKFZP564O0463 protein | 1.9 | other |
| 108859 | AL121500 | Hs.178904 | ESTs | 1.6 | TM |
| 108872 | H06720 | Hs.111680 | endosulfine alpha | 2.2 | other |
| 108891 | AI801235 | Hs.48480 | ESTs | 5.4 | other |
| 108894 | AK001431 | Hs.5105 | hypothetical protein FLJ10569 | 4.1 | TM |
| 108955 | AA149754 | Hs.195155 | Homo sapiens amino acid transport system | 5.7 | ? |
| 108982 | AA151708 | Hs.171980 | homeo box (expressed in ES cells) 1 | 1.7 | other |
| 108987 | AA152178 | Hs.23467 | hypothetical protein FLJ10633 | 6.3 | other |
| 109002 | AB028987 | Hs.72134 | KIAA1064 protein | 1.7 | other |
| 109011 | AA156542 | Hs.72127 | ESTs | 1.5 | other |
| 109026 | AA157811 | | gb:zo35d07.s1 Stratagene colon (937204) | 5.4 | other |
| 109068 | AA164293 | Hs.72545 | ESTs | 3 | other |
| 109101 | AW608930 | Hs.52164 | hypothetical protein FLJ20618 | 1.6 | SS, |
| 109112 | AW419196 | Hs.257924 | hypothetical protein FLJ13782 | 3.3 | TM |
| 109124 | AK000684 | Hs.183887 | hypothetical protein FLJ22104 | 1.7 | other |
| 109139 | AJ132592 | Hs.59757 | zinc finger protein 281 | 2.7 | other |
| 109166 | AA219691 | Hs.73625 | RAB6 interacting, kinesin-like (rabkines | 3 | TM |
| 109198 | BE566742 | Hs.58169 | highly expressed in cancer, rich in leuc | 2.1 | other |
| 109213 | NM_016603 | Hs.82035 | potential nuclear protein C5ORF5; GAP-li | 5.4 | other |
| 109220 | AW958181 | Hs.189998 | ESTs | 5.8 | other |
| 109233 | AU077281 | Hs.170285 | nucleoporin 214kD (CAIN) | 5.3 | other |
| 109270 | N99673 | Hs.3585 | ESTs, Weakly similar to AF126743 1 DNAJ | 1.4 | other |
| 109273 | AA375752 | Hs.82719 | Homo sapiens mRNA; cDNA DKFZp586F1822 (f | 3 | other |
| 109313 | AF153201 | Hs.86276 | C2H2 (Kruppel-type) zinc finger protein | 1.3 | other |
| 109341 | AA213506 | Hs.115099 | EST | 3 | ? |
| 109391 | AL096858 | Hs.184245 | KIAA0929 protein Msx2 interacting nuclea | 1.5 | other |
| 109420 | H83603 | Hs.40408 | homeo box C9 | 2.2 | SS, |
| 109426 | N30531 | Hs.42215 | protein phosphatase 1, regulatory subuni | 3.1 | TM |
| 109429 | AI160029 | Hs.61438 | ESTs | 2 | |
| 109445 | AA232103 | Hs.189915 | ESTs | 1.8 | other |
| 109450 | AB032969 | Hs.173042 | KIAA1143 protein | 3.8 | other |
| 109468 | NM_015310 | Hs.6763 | KIAA0942 protein | 3.3 | other |
| 109478 | AW074143 | Hs.87134 | ESTs | 2 | TM |
| 109570 | L40027 | Hs.118890 | glycogen synthase kinase 3 alpha | 2.1 | other |
| 109662 | F02614 | Hs.27319 | ESTs | 1.4 | other |
| 109825 | R71264 | Hs.16798 | ESTs | 1.3 | other |
| 110039 | H11938 | Hs.21907 | histone acetyltransferase 2 | other | |
| 110056 | AA503041 | Hs.279009 | matrix Gla protein | 2.5 | other |
| 110085 | AA603840 | Hs.29956 | KIAA0460 protein | 1.7 | other |
| 110110 | T07353 | Hs.7948 | ESTs | 2.9 | other |
| 110129 | R51853 | Hs.226429 | ESTs, Weakly similarto ALU1_HUMAN ALU S | 1.7 | SS, |
| 110154 | NM_014521 | Hs.176675 | H3-domain binding protein 4 | 4.3 | other |
| 110240 | AI668594 | Hs.176588 | ESTs, Weakly similar to CP4Y_HUMAN CYTOC | 4.3 | ? |
| 110242 | N41744 | Hs.19978 | CGI-30 protein | 1.3 | other |
| 110259 | H28428 | Hs.32406 | ESTs, Weakly similar to 138022 hypotheti | 2.2 | other |
| 110312 | BE256986 | Hs.11896 | hypothetical protein FLJ12089 | 2.1 | other |
| 110501 | H55748 | | gb:yq94a01.s1 Scares fetal liver spleen | 6.1 | ? |
| 110504 | H55915 | Hs.210859 | hypothetical protein FLJ11016 | 6.1 | TM |
| 110525 | H57330 | Hs.37430 | EST | 6.4 | other |
| 110568 | AK001160 | Hs.5999 | hypothetical protein FLJ10298 | 1.3 | ? |
| 110699 | T97586 | Hs.18090 | ESTs | 1.8 | other |
| 110705 | AB007902 | Hs.32168 | KIAA0442 protein | 1.6 | TM |
| 110742 | AW190338 | Hs.28029 | hypothetical protein MGC11256 | 7.8 | other |
| 110761 | AL138077 | Hs.16157 | hypothetical protein FLJ12707 | 2.5 | other |
| 110762 | BE044245 | Hs.30011 | hypothetical protein MGC2963 | 9.3 | ? |
| 110765 | AK000322 | Hs.18457 | hypothetical protein FLJ20315 | 5.5 | SS, |
| 110769 | BE000831 | Hs.23837 | Homo sapiens cDNA FLJ11812 fis, clone HE | 2.1 | TM |
| 110799 | AI089660 | Hs.323401 | dpy-30-like protein 1.5 | TM | |
| 110805 | T25829 | Hs.24048 | FK506 binding protein precursor | 6.7 | TM |
| 110813 | AA767373 | Hs.35669 | ESTs, Moderately similar to ALU1_HUMAN A | 5.7 | other |
| 110820 | R33261 | Hs.6614 | ESTs, Weakly similar to A43932 mucin 2 p | 3.4 | other |
| 110840 | N31598 | Hs.12727 | hypothetical protein FU21610 | 1.7 | TM |
| 110844 | AI740792 | Hs.167531 | methylcrotonoyl-Coenzyme A carboxylase 2 | 1.7 | other |
| 110854 | BE612992 | Hs.27931 | hypothetical protein FLJ10607 similar to | 4.7 | other |
| 110856 | AA992380 | | gb:ot37g06.s1 Soares_testis_NHT Homo sap | 2.3 | other |
| 110885 | BE384447 | Hs.16034 | hypothetical protein MGC13186 | 3.5 | ? |
| 110897 | AL117430 | Hs.6880 | DKFZP4340156 protein | 2.2 | ? |
| 110915 | BE092285 | Hs.29724 | hypothetical protein FLJ13187 | 2.6 | SS, |
| 110918 | H04360 | Hs.24283 | ESTs, Moderately similar to reduced expr | 1.9 | TM |
| 110958 | NM_005864 | Hs.24587 | signal transduclion protein (SH3 contain | 6.7 | other |
| 110963 | AK002180 | Hs.11449 | DKFZP5640123 protein | 2 | other |
| 110981 | AK001980 | Hs.24284 | ADP-ribosyltransferase (NAD+; poly(ADP-r | 1.3 | other |
| 110984 | AW613287 | Hs.80120 | UDP-N-acetyl-alpha-D-galactosamine:polyp | 1.8 | ? |
| 111125 | N63823 | Hs.269115 | ESTs, Moderately similar to Z195_HUMAN Z | 3.7 | other |
| 111132 | AB037807 | Hs.83293 | hypothetical protein | 2.1 | TM |
| 111164 | N46180 | Hs.122489 | Homo sapiens cDNA FLJ13289 fis, clone OV | 2.3 | other |
| 111172 | R67419 | Hs.21851 | Homo sapiens cDNA FLJ12900 fis, clone NT | 3.7 | other |
| 111174 | AL050166 | Hs.26295 | Homo sapiens mRNA; cDNA DKFZp586D1122 (f | 7.5 | other |
| 111179 | AK000136 | Hs.10760 | asporin (LRR class 1) | 7.1 | other |
| 111184 | AI815486 | Hs.243901 | Homo sapiens cDNA FLJ20738 fis, clone HE | 6.8 | other |
| 111189 | N67603 | Hs.272130 | ESTs, Weakly similar to S65824 reverse t | 3.6 | SS, |
| 111216 | AW139408 | Hs.152940 | ESTs | 1.5 | other |
| 111221 | AB037782 | Hs.15119 | KIAA1361 protein | 2.6 | other |
| 111223 | AA852773 | Hs.334838 | KIAA1866 protein | 4.7 | other |
| 111239 | N90956 | Hs.17230 | hypothetical protein FLJ22087 | 7.9 | ? |
| 111285 | AA778711 | Hs.4310 | eukaryotic translation initiation factor | 7 | other |
| 111299 | AB033091 | Hs.74313 | KIAA1265 protein | 5 | other |
| 111312 | AI523913 | Hs.34504 | ESTs | 3.8 | other |
| 111318 | T99755 | Hs.334728 | ESTs | 1.2 | TM |
| 111337 | AA837396 | Hs.263925 | LIS1-interacting protein NUDE1, rat homo | 5.1 | other |
| 111352 | H58589 | Hs.35156 | Homo sapiens cDNA FLJ11027 fis, clone PL | 2.2 | other |
| 111370 | AI478658 | Hs.94631 | brefeldin A-inhibited guanine nucleotide | 2.8 | ? |
| 111384 | N94606 | Hs.288969 | HSCARG protein | 2.2 | other |
| 111389 | AK000987 | Hs.169111 | oxidation resistance 1 | 2.1 | other |
| 111452 | R02354 | Hs.15999 | ESTs | 2.7 | TM |
| 111486 | AI051194 | Hs.227978 | EST | 6.6 | other |
| 111549 | W90638 | Hs.20321 | ESTs, Moderately similar to ZRF1_HUMAN Z | 1.4 | other |
| 111585 | R10720 | Hs.20670 | EST | 1.6 | ? |
| 111627 | R52656 | Hs.21691 | ESTs | 1.6 | other |
| 111870 | AB037834 | Hs.18685 | Homo sapiens mRNA for KIAA1413 protein, | 2.4 | other |
| 111937 | BE298665 | Hs.14846 | Homo sapiens mRNA; cDNA DKFZp5640016 (fr | 10.6 | other |
| 111944 | AW083791 | Hs.21263 | suppressor of potassium transport defect | 6.6 | TM |
| 111987 | NM_015310 | Hs.6763 | KIAA0942 protein | 5.1 | other |
| 112134 | R41823 | Hs.7413 | ESTs; calsyntenin-2 | 2.8 | other |
| 112244 | AB029000 | Hs.70823 | KIAA1077 protein | 14.6 | other |
| 112388 | R46071 | Hs.301693 | Homo sapiens, clone IMAGE:3638994, mRNA, | 9 | other |
| 112456 | NM_016248 | Hs.232076 | A kinase (PRKA) anchor protein 11 | 1.4 | other |
| 112464 | AW007287 | Hs.28538 | Homo sapiens cDNA: FLJ21086 fis, clone C | 1.4 | TM |
| 112506 | AI742756 | Hs.26079 | ESTs | 3.2 | other |
| 112513 | R68425 | Hs.13809 | hypothetical protein FLJ10648 | 2 | TM |
| 112752 | AK001635 | Hs.14838 | hypothetical protein FLJ10773 | 1.8 | other |
| 112884 | AK000004 | Hs.5013 | Homo sapiens mRNA for FLJ00004 protein, | 6.6 | other |
| 112923 | T10258 | Hs.5037 | EST | 1.5 | ? |
| 112936 | AW970826 | Hs.6185 | KIAA15S7 protein | 3.2 | other |
| 112958 | R61388 | Hs.6724 | ESTs | 6.1 | other |
| 112966 | Z44718 | Hs.102548 | glucocorticoid receptor DNA binding fact | 6.5 | other |
| 112978 | AK000272 | Hs.7099 | hypothetical protein FLJ20265 | 1.2 | other |
| 112995 | AA737033 | Hs.7155 | ESTs, Moderately similar to 2115357A TYK | 5.6 | other |
| 112996 | BE276112 | Hs.7165 | zinc finger protein 259 | 2 | other |
| 113047 | AI571940 | Hs.7549 | ESTs | 1.9 | other |
| 113049 | AW965190 | Hs.7560 | Homo sapiens mRNA for KIAA1729 protein, | 2.4 | TM |
| 113089 | T40707 | Hs.270862 | ESTs | 1.3 | SS, |
| 113196 | T57317 | | gb:yb51a03.s1 Stratagene fetal spleen (9 | 1.7 | other |
| 113248 | T63857 | | gb:yc16e01.s1 Stratagene lung (937210) H | 2.8 | other |
| 113254 | AK002180 | Hs.11449 | DKFZP564O123 protein | 1.3 | other |
| 113277 | AW971049 | Hs.11774 | protein (peptidyl-prolyl cis/trans isome | 3.2 | other |
| 113429 | AA688021 | Hs.179808 | ESTs | 1.2 | other |
| 113499 | AI487908 | Hs.8882 | ESTs | 6 | other |
| 113647 | H59588 | Hs.15233 | ESTs | 2 | SS, |
| 113647 | AA813887 | Hs.188173 | Homo sapiens cDNA FLJ12187 fis, clone MA | 1.3 | SS, |
| 113702 | T97307 | | gb:ye53h05.s1 Soares fetal liver spleen | 4.4 | other |
| 113759 | AW499665 | Hs.9456 | SWI/SNF related, matrix associated, acti | 1.2 | other |
| 113777 | 6E266947 | Hs.10590 | zinc finger protein 313 | 13.4 | other |
| 113783 | AL359588 | Hs.7041 | hypothetical protein DKFZp762B226 | 1.7 | other |
| 113791 | AI269096 | Hs.135578 | chitobiase, di-N-acetyl- | 1.3 | other |
| 113808 | W44735 | Hs.9286 | Homo sapiens cDNA: FLJ21278 fis, clone C | 3.3 | other |
| 113811 | 8E207480 | Hs.6994 | Homo sapiens cDNA: FLJ22044 fis, clone H | 3.1 | other |
| 113817 | H13325 | Hs.332795 | hypothetical protein DKFZp761O17121 | 3.2 | other |
| 113826 | AW378212 | Hs.24809 | hypothetical protein FLJ10826 | 2.3 | ? |
| 113834 | T26483 | Hs.6059 | EGF-containing fibulin-like extracellula | 11.3 | TM |
| 113868 | W57902 | Hs.90744 | proteasome (prosome, macropain) 26S subu | 2.7 | other |
| 113870 | AL079314 | Hs.16537 | hypothetical protein, similar to (U06944 | 6.1 | other |
| 113885 | AW959486 | Hs.21732 | ESTs | 6.6 | other |
| 113923 | AW953484 | Hs.3849 | hypothetical protein FLJ22041 similar to | 1.9 | ? |
| 113989 | W87544 | Hs.268828 | ESTs | 1.2 | other |
| 114022 | AI539519 | Hs.120969 | Homo sapiens cDNA FLJ11562 fis, clone HE | 5.4 | other |
| 114030 | AI825386 | Hs.164478 | hypothetical protein FLJ21939 similar to | 9.4 | other |
| 114060 | AB029551 | Hs.7910 | RING1 and YY1 binding protein | 1.8 | other |
| 114196 | AF017445 | Hs.150926 | fucose-1-phosphate guanylyltransferase | 1.5 | other |
| 114226 | AB028968 | Hs.7989 | KIAA1045 protein | 1.8 | other |
| 114253 | BE149866 | Hs.14831 | Homo sapiens, Similar to zinc finger pro | 2.3 | other |
| 114262 | AL117518 | Hs.3686 | KIAA0978 protein | 1.4 | TM |
| 114275 | AW515443.comp | Hs.306117 | KIAA0306 protein | 15.8 | othe |
| 114292 | AI815395 | Hs.184641 | fatty acid desaturase 2 | 1.9 | TM |
| 114309 | AA332453 | Hs.20824 | CGI-85 protein | 2.4 | other |
| 114392 | AA249590 | Hs.100748 | ESTs, Weakly similar to A28996 proline-r | 1.9 | other |
| 114407 | BE539976 | Hs.103305 | Homo sapiens mRNA; cDNA DKFZp434B0425 (f | 1.3 | TM |
| 114455 | H37908 | Hs.271616 | ESTs, Weakly similar to ALU8_HUMAN ALU S | 5.6 | other |
| 114463 | AL120247 | Hs.40109 | KIAA0872 protein | 5.3 | TM |
| 114464 | AI091713 | Hs.106597 | Homo sapiens, Similar to RIKEN cDNA 1110 | 1.3 | other |
| 114471 | AA028074 | Hs.104613 | RP42 homolog | 1.9 | ? |
| 114480 | BE066778 | Hs.151678 | UDP-N-acetyl-alpha-D-galactosamine:polyp | 13.4 | other |
| 114671 | AA766268 | Hs.266273 | hypothetical protein FLJ13346 | 2 | other |
| 114698 | AA476966 | Hs.110857 | polymerase (RNA) III (DNA directed) poly | 3.6 | other |
| 114730 | AI373544 | Hs.331328 | intermediate filament protein syncoilin | 3.9 | other |
| 114767 | AI859865 | Hs.154443 | minichromosome maintenance deficient (S. | 1.7 | other |
| 114774 | AV656017 | Hs.184325 | CGI-76 protein | 3.2 | other |
| 114798 | AA159181 | Hs.54900 | serologicelly defined colon cancer anlig | 3.6 | other |
| 114860 | AL157545 | Hs.42179 | bromodomain and PHD finger containing, 3 | 4.4 | other |
| 114895 | AA236177 | Hs.76591 | KIAA0887 protein | 7.2 | other |
| 114896 | BE539101 | Hs.5324 | hypothetical protein | 1.3 | other |
| 114911 | AA236672 | | gb:zt29f02.s1 Soares ovary tumor NbHOT H | 1.5 | other |
| 114930 | AA237022 | Hs.188717 | ESTs | 2 | SS, |
| 114938 | AA242834 | Hs.58384 | ESTs | 2.9 | other |
| 114965 | AI733881 | Hs.72472 | BMP-R1B | 2.3 | ? |
| 115023 | AF102546 | Hs.63931 | dachshund (Drosophila) homolog | 1.3 | other |
| 115038 | AA252360 | Hs.87968 | toll-like receptor 9 | 1.6 | other |
| 115061 | AI751438 | Hs.41271 | Homo sapiens mRNA full length insert cDN | 11.8 | other |
| 115117 | AI670847 | Hs.5324 | hypothetical protein | 1.5 | other |
| 115206 | AW183695 | Hs.186572 | ESTs | 2.5 | other |
| 115221 | AW365434 | Hs.79741 | hypothetical protein FLJ10116 | 1.5 | other |
| 115239 | BE251328 | Hs.73291 | hypothetical protein FLJ10881 | 1.3 | TM |
| 115242 | AI368236 | Hs.283732 | ESTs, Moderately similar to ALU1_HUMAN A | 1.4 | other |
| 115278 | AK002163 | Hs.301724 | hypothetical protein FLJ11301 | 1.5 | other |
| 115285 | AW972872 | Hs.293736 | ESTs | 2.4 | other |
| 115291 | BE545072 | Hs.122579 | hypothetical protein FLJ10461 | 6.3 | SS, |
| 115400 | AI215069 | Hs.89113 | ESTs | 6.7 | ? |
| 115468 | AA314349 | Hs.48499 | tumor antigen SLP-8p | 7.5 | ? |
| 115471 | AK001376 | Hs.59346 | hypothetical protein FLJ105I4 | 1.4 | TM |
| 115479 | AW301608 | Hs.278188 | ESTs, Moderately similar to 154374 gene | 4.1 | TM |
| 115496 | AW247593 | Hs.71819 | eukaryotic translation initiation factor | 16.3 | other |
| 115500 | Y14443 | Hs.88219 | zinc finger protein 200 | 5 | other |
| 115553 | AJ275986 | Hs.71414 | transcription factor (SMIF gene) | 2.5 | other |
| 115581 | AI540842 | Hs.61082 | ESTs | 6.2 | other |
| 115587 | BE081342 | Hs.283037 | HSPC039 protein | 2.9 | other |
| 115590 | AA399477 | Hs.67896 | 7-60 protein | 5.3 | TM |
| 115646 | N36110 | Hs.305971 | solute carrier family 2 (facilitated glu | 4.8 | ? |
| 115652 | BE093589 | Hs.38178 | hypothetical protein FLJ23468 | 10.6 | other |
| 115655 | AL048269 | Hs.288544 | Homo sapiens, clone MGC:16063, mRNA, com | 12.7 | TM |
| 115663 | AI138785 | Hs.40507 | ESTs | 2 | other |
| 115676 | AA953006 | Hs.88143 | ESTs | 3.1 | other |
| 115690 | AA625132 | Hs.44159 | hypothetical protein FLJ21615 | 1.7 | TM |
| 115693 | AF231023 | Hs.55173 | cadherin, EGF LAG seven-pass G-type rece | 6.9 | other |
| 115715 | BE395161 | Hs.1390 | proteasome (prosome, macropain) subunit, | 1.7 | other |
| 115734 | AI950339 | Hs.40782 | ESTs | 2.7 | TM |
| 115811 | NM_015434 | Hs.48604 | DKFZP434B168 protein | 2.1 | other |
| 115823 | AI732742 | Hs.87440 | ESTs | 2.1 | other |
| 115837 | AI675217 | Hs.42761 | ESTs | 1.3 | other |
| 115844 | AI373062 | Hs.332938 | hypothetical protein MGC5370 | 4.4 | other |
| 115866 | AW062629 | Hs.52081 | KIAA0867 protein | 7.3 | other |
| 115875 | N55669 | Hs.333823 | mitochondrial ribosomal protein L13 | 1.2 | other |
| 115941 | AI867451 | Hs.46679 | hypothetical protein FLJ20739 | 5.5 | other |
| 115968 | AB037753 | Hs.62767 | KIAA1332 protein | 9.8 | other |
| 116003 | BE275469 | Hs.66493 | Down syndrome cntical region gene 5 | 1.4 | other |
| 116011 | AL359053 | Hs.57664 | Homo sapiens mRNA full length insert cDN | 2.4 | other |
| 116108 | AA770688 | Hs.28777 | H2A histone family, member L | 1.8 | other |
| 116134 | BE243834 | Hs.50441 | CGI-04 protein | 1.4 | other |
| 116189 | N35719 | Hs.44749 | ESTs, Moderately similar to T00358 hypot | 1.2 | other |
| 116195 | AW821113 | Hs.72402 | ESTs | 2.1 | other |
| 116238 | AV660717 | Hs.47144 | DKFZP586N0819 protein | 1.7 | other |
| 116246 | AF265555 | Hs.250646 | baculoviral IAP repeat-containing 6 | 1.7 | other |
| 116262 | AI936442 | Hs.59838 | hypothetical protein FLJ10808 | 1.8 | ? |
| 116298 | AI955411 | Hs.94109 | Homo sapiens cDNA FLJ13634 fis, clone PL | 1.9 | other |
| 116318 | AF097645 | Hs.58570 | deleted in cancer 1; RNA helicase HOB/DI | 5 | SS, |
| 116325 | AI472106 | Hs.49303 | Homo sapiens cDNA FLJ11663 fis, clone HE | 1.4 | SS, |
| 116336 | AL133033 | Hs.4084 | KIAA1025 protein | 1.9 | ? |
| 116339 | AK000290 | Hs.44033 | dipeptidyl peptidase 8 | 1.5 | other |
| 116350 | AA497129 | Hs.184771 | nuclear factor I/C (CCAAT-binding transc | 1.9 | ? |
| 116358 | AI149586 | Hs.38125 | interferon-induced protein 75, 52kD | 1.9 | ? |
| 116365 | N50174 | Hs.46765 | ESTs | 6.1 | other |
| 116368 | N90466 | Hs.71109 | KIAA1229 protein | 1.6 | ? |
| 118417 | AW499664 | Hs.12484 | Human clone 23826 mRNA sequence | 7.4 | other |
| 116436 | AA161411 | Hs.58668 | chromosome 21 open reading frame 57 | 2.1 | other |
| 116462 | AF218313 | Hs.236828 | putative helicase RUVBL | 1.5 | TM |
| 116470 | AI272141 | Hs.83484 | SRY (sex determining region Y)-box 4 | 2.1 | TM |
| 116575 | AA312572 | Hs.6241 | phosphoinositide-3-kinase, regulatory su | 1.5 | other |
| 116637 | AK001043 | Hs.92033 | integrin-linked kinase-associated serine | 2.7 | other |
| 116640 | X89984 | Hs.211563 | B-cell CLL/lymphoma 7A | 2.3 | other |
| 116700 | AI800202 | Hs.317589 | hypothetical protein MGC10765 | 1.4 | other |
| 116705 | AW074819 | Hs.12313 | hypothetical protein FLJ14566 | 3.4 | other |
| 116732 | AW152225 | Hs.165909 | ESTs, Weakly similar to I38022 hypotheti | 2.9 | other |
| 116926 | H73608 | Hs.290830 | ESTs | 1.7 | TM |
| 117034 | U72209 | Hs.180324 | YY1-asscoiated factor 2 | 3.4 | TM |
| 117132 | AI393666 | Hs.42315 | p10-binding protein | 5.2 | ? |
| 117247 | N21032 | | gb:yx46f06.s1 Soares melanocyte 2NbHM Ho | 5.5 | TM |
| 117276 | N71183 | Hs.121806 | Homo sapiens cDNA FLJ11971 fis, clone HE | 1.5 | TM |
| 117284 | AK001701 | Hs.183779 | Homo sapiens cDNA FLJ10590 fis, clone NT | 2 | other |
| 117367 | AI041793 | Hs.42502 | ESTs | 2 | other |
| 117368 | AI878942 | Hs.90336 | ATPase, H+ transporting, lysosomal (vacu | 2.1 | ? |
| 117382 | AF150275 | Hs.40173 | ESTs | 2.7 | TM |
| 117412 | N32536 | Hs.42645 | solute carrier family 16 (monocarboxylic | 1.4 | other |
| 117557 | AF123050 | Hs.44532 | diubiquitin | 3.4 | TM |
| 117568 | N34895 | Hs.44648 | ESTs | 3.4 | ? |
| 117745 | BE294925 | Hs.46680 | CGI-12 protein | 3 | SS, |
| 117754 | AA121673 | Hs.59757 | zinc linger protein 281 | 1.9 | other |
| 117879 | N54706 | Hs.303025 | chromosome 11 open reading frame 24 | 1.8 | other |
| 117904 | BE540675 | Hs.332938 | hypothetical protein MGC5370 | 6 | ? |
| 117911 | AL137379 | Hs.47125 | hypothetical protein FLJ13912 | 1.7 | other |
| 117933 | Y10518 | Hs,116470 | hypothetical protein FLJ20048 | 1.7 | other |
| 117983 | AL110246 | Hs.47367 | KIAA1785 protein | 5.4 | other |
| 118078 | N54321 | Hs.47790 | EST | 5.2 | other |
| 118301 | AA453902 | Hs.293264 | ESTs | 2.6 | other |
| 118429 | AA243332 | Hs.74649 | cytochrome c oxidase subunit VIc | 2.5 | TM |
| 118472 | AL157545 | Hs.42179 | bromodomain and PHD finger containing, 3 | 4.1 | other |
| 118488 | AJ277275 | Hs.50102 | rapa-2 (rapa gene) | 1.2 | other |
| 118509 | N22617 | Hs.43228 | Homo sapiens cDNA FLJ11835 fis, clone HE | 1.5 | other |
| 118528 | AI949952 | Hs.49397 | ESTs | 7.4 | ? |
| 118656 | AI458020 | Hs.293287 | ESTs | 2.5 | other |
| 118670 | AA332845 | Hs.152618 | ESTs, Moderately similar to ZN91_HUMAN Z | 1.2 | TM |
| 118698 | A6033113 | Hs.50187 | KIAA1287 protein | 2.1 | TM |
| 118737 | AA199686 | | gb:zq75g09.r1 Stratagene hNT neuron (937 | 5.2 | other |
| 118925 | N92293 | Hs.206832 | ESTs, Moderately similar to ALU8_HUMAN A | 1.4 | other |
| 118984 | AI668709 | Hs.240722 | ESTs, Moderately similar to ALU8_HUMAN A | 3.6 | other |
| 118986 | AF148713 | Hs.125830 | bladder cancer overexpressed protein | 4.9 | ? |
| 119206 | W24781 | Hs.293798 | KIAA1710 protein | 1.7 | TM |
| 119235 | AW453069 | Hs.3657 | activity-dependent neuroprotective prote | 2.2 | other |
| 119265 | 8E539706 | Hs.285363 | ESTs | 1.4 | ? |
| 119279 | N57568 | Hs.48028 | EST | 25.1 | other |
| 119298 | NM_001241 | Hs.155478 | cyclinT2 | 1.6 | ? |
| 119338 | AI417240 | Hs.320836 | ESTs, Weakly similar to A47582 B-cell gr | 1.3 | other |
| 119403 | AL117554 | Hs.119908 | nucleolar protein NOP5/NOP58 | 6.7 | TM |
| 119478 | AI624342 | Hs.170042 | ESTs | 2.4 | other |
| 119486 | AI796730 | Hs.55513 | ESTs | 2.1 | other |
| 119513 | W37933 | | Empirically selected from AFFX single pr | 1.9 | other |
| 119601 | AK000155 | Hs.91684 | Homo sapiens mRNA; cDNA DKFZp6671103 (fr | 3.7 | TM |
| 119602 | AW675298 | Hs.233694 | hypothetical protein FLJ11350 | 3 | other |
| 119676 | AA243837 | Hs.57787 | ESTs | 1.4 | other |
| 119682 | W61019 | Hs.57811 | ESTs | 1.2 | ? |
| 119774 | AB032977 | Hs.6298 | KIAA1151 protein | 1.8 | TM |
| 119780 | NM_016625 | Hs.191381 | hypothetical protein | 3.1 | other |
| 119789 | BE393948 | Hs.50915 | kallikrein 5 (KLK5; KLK-L2; stratum com | 9.2 | other |
| 119805 | AJ223810 | Hs.43213 | ESTs, Weakly similar to IEFS_HUMAN TRANS | 3.6 | TM |
| 119818 | AA130970 | Hs.58382 | hypothetical protein FLJ1101 | 2.5 | ? |
| 119863 | AA081218 | Hs.58608 | Homo sapiens cDNA FLJ14206 fis, clone NT | 2.7 | TM |
| 119905 | AW449064 | Hs.119571 | collagen, type III, alpha 1 (Ehlers-Dani | 2.6 | other |
| 119966 | AA703129 | Hs.58963 | ESTs | 2.7 | other |
| 120132 | W57554 | Hs.125019 | lymphoid nuclear protein (LAF-4) mRNA | 1.2 | other |
| 120206 | H26735 | Hs.91668 | Homo sapiens clone PP1498 unknown mRNA | 45.7 | other |
| 120248 | AI924294 | Hs.173259 | uncharacterized bone marrow protein BM03 | 1.2 | other |
| 120269 | AW131940 | Hs.104030 | ESTs | 9.6 | other |
| 120274 | AA177051 | | gb:nc02a02.s1 NCI_CGAP_Pr3 Homo sapiens | 4.7 | other |
| 120280 | AA190577 | | gb:zp52g02.s1 Stratagene HeLa call s393 | 2.1 | other |
| 120296 | AW995911 | Hs.299883 | hypothetical protein FLJ23399 | 1.9 | TM |
| 120297 | AA191384 | Hs.104072 | ESTs, Weakly similar to Z195_HUMAN ZINC | 15.2 | other |
| 120324 | AA195517 | Hs.191643 | ESTs | 5.6 | ? |
| 120325 | AA195651 | Hs.104106 | ESTs | 6.5 | other |
| 120327 | AK000292 | Hs.278732 | hypothetical protein FLJ20285 | 16.1 | other |
| 120336 | N85785 | Hs.181165 | eukaryotic translation elongation factor | 3 | other |
| 120342 | AW450669 | Hs.45068 | hypothetical protein DKFZp4341143 | 5.8 | other |
| 120345 | AA210722 | Hs.104158 | ESTs | 4.6 | SS, TM |
| 120349 | AW969481 | Hs.55189 | hypothetical protein | 16.8 | other |
| 120352 | R06859 | Hs.193172 | ESTs, Weakly similar to 138022 hypotheti | 5.1 | other |
| 120356 | AF000545 | Hs.296433 | putative purinergic receptor | 28.1 | TM |
| 120371 | AA219305 | Hs.104196 | EST | 12.4 | ? |
| 120382 | AA228026 | Hs.38774 | ESTs | 4.1 | TM |
| 120383 | AL109963 | Hs.123122 | FSH primary response (LRPR1, rat) homolo | 9.7 | TM |
| 120386 | AW969665 | Hs.154848 | hypothetical protein DKFZp43400127 | 32.6 | other |
| 120388 | AA232874 | Hs.104245 | ESTs | 3.2 | other |
| 120389 | AW967985 | Hs.325572 | ESTs, Moderately similar to ALU7_HUMAN A | 21.7 | other |
| 120396 | AA134006 | Hs.79306 | eukaryotic translation initiation factor | 12.5 | other |
| 120404 | AB023230 | Hs.96427 | KIM1013 protein | 7.3 | other |
| 120418 | AW966893 | Hs.26613 | Homo sapiens mRNA; cDNA DKFZp586F1323 (f | 11.4 | other |
| 120423 | AA236453 | Hs.18978 | Homo sapiens cDNA: FLJ22822 fis, clone K | 1.9 | other |
| 120472 | AI950087 | | gbwq05c02.x1 NCI_CGAP_Kid12 Homo sapien | 19.4 | other |
| 120473 | AA251973 | Hs.269988 | ESTs | 5.5 | ? |
| 120484 | AA253170 | Hs.96473 | EST | 10.4 | ? |
| 120504 | AA256837 | | gb:zr84d10.s1 Soares_NhHMPu_S1 Homo sapi | 4 | ? |
| 120509 | BE047718 | Hs.96545 | ESTs | 9.4 | other |
| 120520 | AA258601 | Hs.161731 | EST | 2.4 | other |
| 120535 | BE350244 | Hs.96547 | ESTs | 2.5 | ? |
| 120551 | AA279160 | Hs.111407 | Homo sapiens, clone IMAGE:3613029, mRNA, | 5.3 | other |
| 120570 | AA280679 | Hs.271445 | ESTs, Weakly similar to ALU1_HUMAN ALU S | 14.4 | ? |
| 120582 | BE244830 | Hs.284228 | ZNF135-like protein | 10.2 | ? |
| 120590 | AW372799 | Hs.125790 | leucine-rich repeat-containing 2 | 2.2 | ? |
| 120596 | AA282074 | Hs.237323 | N-acetylglucosamine-phosphate mutase | 7.6 | other |
| 120619 | AW965339 | Hs.111471 | ESTs | 2.5 | other |
| 120624 | AW407987 | Hs.173518 | M-phase phosphoprotein homolog | 52 | other |
| 120639 | AA286942 | | gb:zs56f05.s1 NCI_CGAP_GCB1 Homo sapiens | 2.4 | other |
| 120648 | AA287095 | Hs.140309 | Homo sapiens, done IMAGE:3677194, mRNA, | 5 | other |
| 120653 | AW063659 | Hs.191649 | ESTs | 2.2 | other |
| 120668 | AW969638 | Hs.112318 | 6.2 kd protein | 2.2 | TM |
| 120669 | BE536739 | Hs.109909 | ESTs | 1.9 | TM |
| 120695 | AA976503 | | gb:oq30a04.s1 NCI_CGAP_GC4 Homo sapiens | 46.8 | TM |
| 120696 | AI821539 | Hs.97249 | ESTs | 2.5 | other |
| 120713 | AW449855 | Hs.96557 | Homo sapiens cDNA FLJ12727 fis, clone NT | 6 | other |
| 120718 | AA292747 | Hs.97296 | ESTs | 2.9 | other |
| 120750 | AI191410 | Hs.96693 | ESTs, Moderately similar to 2109260A Bc | 7.1 | SS, |
| 120774 | AI608909 | Hs.193985 | ESTs | 7.9 | other |
| 120807 | AA346385 | Hs.30002 | SH3-containing protein SH3GLB2; KIAA1848 | 7 | TM |
| 120809 | AA346495 | | gb:EST52657 Fetal heart II Homo sapiens | 4.5 | other |
| 120938 | AA386260 | Hs.104632 | EST | 4.5 | ? |
| 120977 | AA398155 | Hs.97600 | ESTs | 4.5 | other |
| 120984 | BE262951 | Hs.99052 | ESTs | 5.6 | other |
| 120985 | AI219896 | Hs.97592 | ESTs | 1.3 | other |
| 121011 | AA398360 | Hs.97608 | EST | 3.2 | other |
| 121026 | AI439713 | Hs.165295 | ESTs | 3.6 | other |
| 121081 | AA398721 | Hs.186749 | ESTs, Highly similar to 137550 mismatch | 5.5 | other |
| 121133 | AA363307 | Hs.97032 | ESTs | 3.8 | other |
| 121176 | AL121523 | Hs.97774 | ESTs | 1.7 | TM |
| 121223 | AI002110 | Hs.97169 | ESTs, Weakly similar to dJ667H12.2.1 [H. | 2.9 | other |
| 121320 | AA403008 | Hs.301927 | c6.1A | 1.9 | other |
| 121340 | AW956981 | Hs.97910 | Homo sapiens cDNA FLJ13383 fis, clone PL | 3.5 | other |
| 121408 | AA406137 | Hs.98019 | EST | 6.1 | ? |
| 121439 | AA410190 | Hs.98076 | ESTs, Weakly similar to A47582 B-cell gr | 7.5 | other |
| 121450 | M406430 | Hs.105362 | Homo sapiens, done MGC:18257, mRNA, com | 7.1 | other |
| 121452 | AW971063 | Hs.292882 | ESTs | 1.8 | other |
| 121455 | H58306 | Hs.15165 | retinoic add induced 14 | 10.5 | other |
| 121457 | W07404 | Hs.144502 | hypothetical protein FLJ22055 | 3.5 | TM |
| 121496 | AA442224 | Hs.97900 | ESTs | 14.4 | other |
| 121505 | AA494172 | Hs.194417 | ESTs | 13.1 | other |
| 121508 | AA402515 | Hs.97887 | ESTs | 28 | other |
| 121513 | AA416653 | Hs.181510 | ESTs | 6.3 | other |
| 121514 | AA412112 | | gb:zt69b02.s1 Soares_testis_NHT Homo sap | 2.7 | SS, |
| 121549 | AA412477 | Hs.98142 | EST | 7.5 | ? |
| 121558 | AA412497 | | gb:zt95g12.s1 Soares_testis_NHT Homo sap | 2.8 | other |
| 121577 | AA411970 | Hs.98096 | EST | 3.5 | ? |
| 121581 | AA416568 | | gb:zu05c10.s1 Soares_testis_NHT Homo sap | 6.2 | TM |
| 121589 | AD001528 | Hs.89718 | spermine synthase | 4 | other |
| 121594 | AA626010 | Hs.98247 | ESTs | 2.2 | other |
| 121622 | AA416931 | Hs.126065 | ESTs | 4.3 | TM |
| 121655 | M421537 | Hs.178072 | Homo sapiens mRNA; cDNA DKFZp434B1023 (f | 7.9 | other |
| 121682 | AA418160 | Hs.86043 | Homo sapiens cDNA FLJ13558 fis, clone PL | 2 | other |
| 121690 | AV660305 | Hs.110286 | ESTs | 4.7 | ? |
| 121706 | U55184 | Hs.154145 | hypothetical protein FLJ11585 | 12.7 | other |
| 121714 | AA419225 | Hs.98269 | Homo sapiens cDNA FLJ11953 fis, clone HE | 8.3 | ? |
| 121729 | AI949597 | Hs.98325 | ESTs | 1.8 | TM |
| 121731 | AA421041 | Hs.180744 | ESTs | 4.1 | TM |
| 121744 | AA398784 | Hs.97514 | ESTs | 7.1 | SS, |
| 121748 | BE536911 | Hs.234545 | hypothetical protein NUF2R | 19.5 | other |
| 121773 | AB033022 | Hs.158654 | KIAA1196 protein | 8 | other |
| 121775 | AA421773 | Hs.161008 | ESTs | 1.7 | other |
| 121776 | AA292579 | Hs.125133 | hypothetical protein FLJ22501 | 6.7 | other |
| 121786 | AI810774 | Hs.98376 | ESTs | 10.5 | other |
| 121832 | AW340797 | Hs.98434 | ESTs | 5.9 | other |
| 121836 | AA328348 | Hs.218289 | ESTs | 3.9 | other |
| 121839 | AA425691 | Hs,191606 | ESTs, Highly similar to KIAA1048 protein | 5 | other |
| 121842 | AF027406 | Hs.104865 | serine/threonine kinase 23 | 2.7 | ? |
| 121847 | AA446628 | Hs.2799 | cartilage linking protein 1 | 2.3 | other |
| 121871 | AW972668 | Hs.293044 | ESTs | 2.9 | TM |
| 121882 | AA426376 | Hs.98459 | ESTs | 5 | other |
| 121911 | AA427950 | | gb:zw50f02.s1 Soares_total_fetus_Nb2HF8_ | 7.3 | TM |
| 121915 | AA428179 | Hs.223405 | ESTs, Moderately similar to A46010 X-lin | 2.5 | other |
| 121935 | AA428647 | Hs.98611 | EST | 2.3 | other |
| 121983 | AA298760 | Hs.180191 | hypothetical protein FLJ14904 | 3.4 | other |
| 121985 | AI862570 | Hs.299214 | Homo sapiens, clone IMAGE:2822295, mRNA, | 11.4 | other |
| 121995 | AA210863 | Hs.3532 | nemo-like kinase | 3.8 | ? |
| 121999 | AA430211 | Hs.98668 | EST | 6.5 | other |
| 122009 | AW292763 | Hs.160822 | Homo sapiens CDNA: FLJ20863 fis, clone A | 2.2 | other |
| 122013 | AA431085 | Hs.98706 | ESTs | 6.6 | other |
| 122036 | W92142 | Hs.271963 | ESTs, Weakly similar to ALU5_HUMAN ALU S | 13.1 | other |
| 122050 | AI453076 | Hs.166109 | ELAV (emblyonic lethal, abnormal vision, | 9.1 | other |
| 122060 | AA431738 | Hs.98750 | EST | 13.1 | ? |
| 122114 | AW161023 | Hs.104921 | ESTs | 1.5 | other |
| 122188 | AA398838 | | gb:zt80d01.r1 Soares_testis_NHT Homo sap | 3.4 | other |
| 122204 | AA435936 | Hs.98842 | EST | 5.6 | other |
| 122246 | AA329550 | Hs.29417 | HCF-binding transcription factor Zhangfe | 5.2 | other |
| 122257 | AA436819 | Hs.98899 | ESTs | 5.6 | other |
| 122302 | AA441801 | Hs.104947 | ESTs | 5.8 | other |
| 122341 | AW601969 | Hs.99010 | hypothetical protein FLJ22263 similar to | 2 | other |
| 122356 | AA443794 | Hs.98390 | ESTs | 7.4 | SS, TM |
| 122369 | AA443985 | Hs.303222 | ESTs | 12.2 | ? |
| 122371 | AA868555 | Hs.178222 | ESTs | 5 | ? |
| 122372 | AA446008 | Hs.336677 | EST | 7.8 | ? |
| 122378 | AB032948 | Hs.21356 | hypothetical protein DKFZp762K201S | 2.5 | ? |
| 122405 | AA446572 | Hs.303223 | EST | 2.8 | TM |
| 122412 | AA446869 | Hs.119316 | ESTs | 7.4 | other |
| 122415 | AA446918 | Hs.99088 | EST | 1.9 | other |
| 122418 | AA446966 | Hs.99090 | ESTs, Moderately similar to similar to K | 6.9 | ? |
| 122440 | AW505139 | Hs.9460 | Homo sapiens mRNA; cDNA DKFZp547C244 (fr | 2.6 | other |
| 122446 | AA447603 | Hs.99123 | EST | 1.8 | TM |
| 122448 | AA447626 | Hs.99127 | EST | 3.5 | other |
| 122458 | AI266159 | Hs.104980 | ESTs | 1.5 | other |
| 122460 | AW418788 | Hs.99148 | ESTs, Weakly similarto S43569 R01H10.6 | 9.7 | other |
| 122464 | AA448158 | Hs.99152 | EST | 4.9 | other |
| 122490 | AA448349 | Hs.238151 | EST | 6.2 | ? |
| 122492 | AA448417 | Hs.104990 | ESTs | 5.5 | other |
| 122502 | AA204969 | Hs.234863 | Homo sapiens cDNA FLJ12082 fis, clone HE | 1.3 | other |
| 122510 | AA449232 | Hs.99195 | ESTs | 11.2 | ? |
| 122530 | AW959741 | Hs.40368 | adaptor-related protein complex 1, sigma | 10.1 | other |
| 122547 | AA779725 | Hs.164589 | ESTs | 2.5 | SS, |
| 122555 | AA194055 | Hs.293858 | ESTs | 1.9 | other |
| 122570 | AA452578 | Hs.262907 | ESTs | 9.5 | other |
| 122572 | AA452601 | Hs.99287 | EST | 11 | ? |
| 122586 | AK001910 | Hs.99303 | Homo sapiens cDNA FLJ11048 fis, clone PL | 3.4 | other |
| 122587 | AB040893 | Hs.6968 | KIAA1460 protein | 2 | other |
| 122598 | AI028173 | Hs.99329 | ESTs | 1.7 | ? |
| 122599 | AL355841 | Hs.99330 | hypothetical protein FLJ23588 | 4.4 | ? |
| 122602 | AA411925 | Hs.301960 | ESTs | 4.7 | other |
| 122607 | AA453518 | Hs.98023 | ESTs | 61.5 | other |
| 122614 | AA453630 | Hs.99339 | EST | 10.7 | ? |
| 122616 | AA453638 | Hs.161873 | ESTs | 107.3 | ? |
| 122617 | AI681535 | Hs.148135 | serine/threonine kinase 33 | 121.4 | other |
| 122618 | AA453641 | | gb:zx48e06.s1 Soares_testis_NHT Homo sap | 31.1 | SS, |
| 122622 | AA453987 | Hs.144802 | ESTs | 5.6 | other |
| 122717 | AA456859 | Hs.178358 | ESTs | 8.5 | SS, |
| 122762 | AI376875 | Hs.105119 | ESTs | 10.4 | other |
| 122829 | AW204530 | Hs.99500 | ESTs | 81.8 | ? |
| 122834 | AA461492 | Hs.99545 | Homo sapiens cDNA FLJ10658 fis, clone NT | 3.7 | ? |
| 122836 | AA460581 | Hs.290996 | ESTs | 4.6 | other |
| 122837 | AA461509 | Hs.293565 | ESts, Weakly similar to putative p150 [H | 2.7 | TM |
| 122838 | AA460584 | Hs.334386 | ESTs | 75.3 | other |
| 122854 | AA600235 | Hs.9625 | NIMA (never in mitosis gene a)-related k | 7.8 | other |
| 122856 | AI929374 | Hs.75367 | Src-like-adapter | 5.8 | other |
| 122861 | AA335721 | Hs.119394 | ESTs | 1.3 | other |
| 122866 | BE539656 | Hs.283705 | ESTs | 4.2 | other |
| 122868 | AF005216 | Hs.115541 | Janus kinase 2 (a protein tyrosine kinas | 5.3 | other |
| 122870 | AW576312 | Hs.318722 | Homo sapiens cDNA: FLJ21766 fis, clone C | 9.9 | ? |
| 122872 | AW081394 | Hs.97103 | ESTs | 5.3 | other |
| 122879 | AA769410 | Hs.128654 | ESTs | 13.9 | other |
| 122907 | AA470074 | Hs.169896 | ESTs | 11.5 | other |
| 122916 | AA470140 | Hs.229170 | EST | 1.7 | TM |
| 122981 | AA478951 | Hs.105629 | ESTs | S | other |
| 123013 | AW968324 | Hs.17384 | ESTs | 15.4 | other |
| 123016 | AW338067 | Hs.323231 | Homo sapiens cDNA FLJ11946 fis, clone HE | 2.8 | other |
| 123034 | AL359571 | Hs.44054 | ninein (GSK3B interacting protein) | 8.7 | other |
| 123072 | AI382600 | Hs.104308 | ESTs, Weakly similar to KIAA1395 protein | 8.8 | other |
| 123082 | AA485360 | Hs.105661 | ESTs | 4 | ? |
| 123088 | AI343652 | Hs.105667 | ESTs | 3.8 | other |
| 123110 | AA486256 | Hs.193510 | EST | 7.4 | other |
| 123114 | BE304942 | Hs.265848 | myomegalin | 2.8 | ? |
| 123131 | T52027 | Hs.271795 | ESTs, Weakly similar to I38022 hypotheti | 2.4 | other |
| 123132 | AI061582 | Hs.324179 | Homo sapiens cDNA FLJ12371 fis, done MA | 15.6 | TM |
| 123136 | AW451999 | Hs.194024 | ESTs | 5.2 | other |
| 123149 | AI734179 | Hs.105676 | ESTs | 23.8 | TM |
| 123152 | AW601773 | Hs.270259 | ESTs | 5.2 | other |
| 123258 | AA490929 | Hs.105274 | ESTs, Weakly similar to RMS1_HUMAN REGUL | 9.3 | ? |
| 123315 | AA496369 | | gb:zv37d10.s1 Soares ovary tumor NbHOT H | 4.2 | TM |
| 123369 | AA504757 | Hs.105738 | ESTs | 7 | other |
| 123394 | AA731404 | Hs.105510 | ESTs | 3.7 | other |
| 123433 | AW450922 | Hs.112478 | ESTs | 3.8 | other |
| 123466 | AA599042 | Hs.112503 | EST | 7.4 | other |
| 123470 | AW303285 | Hs.303632 | Human DNA sequence from clone RP11-110H4 | 3.5 | other |
| 123471 | AB021644 | Hs.197219 | zinc finger protein 14 (KOX 6) | 5.2 | ? |
| 123475 | BE439553 | Hs.250528 | Homo sapiens, done IMAGE:4098694, mRNA, | 1.7 | other |
| 123482 | N95059 | Hs.55098 | ESTs | 1.6 | other |
| 123486 | BE019072 | Hs.334802 | Homo sapiens cDNA FLJ14680 fis, clone NT | 2.4 | other |
| 123508 | AW380388 | Hs.155546 | KIAA1080 protein; Golgi-associated, gamin | 2.2 | TM |
| 123615 | AA609170 | | gb:af12a12.s1 Soares_testis_NHT Homo sap | 7.9 | other |
| 123619 | AA602964 | | gb:no97c02.s1 NCI_CGAP_Pr2 Homo sapiens | 2.8 | other |
| 123658 | AA609364 | | gb:zu71d09.s1 Soares_testis_NHT Homo sap | 1.7 | ? |
| 123674 | AI269609 | Hs.105187 | kinesin protein 9 gene | 5.7 | ? |
| 123735 | NM_013241 | Hs.95231 | FH1/FH2 domain-containing protein | 10 | other |
| 123738 | AA609891 | Hs.112777 | EST | 5.2 | other |
| 123753 | AA609955 | Hs.234961 | Huntingtin interacting protein E | 30.6 | TM |
| 123804 | AA620464 | Hs.261915 | EST, Weakly similar to S65657 alpha-1C-a | 2.1 | other |
| 123811 | AA620586 | | gb:ae60g05.s1 Stratagene lung carcinoma | 2.7 | other |
| 123951 | AB012922 | Hs.173043 | metastasis-associated 1-like 1 | 6.3 | ? |
| 123983 | AJ272267 | Hs.146178 | choline dehydrogenase | 4.4 | other |
| 124001 | L42542 | Hs.75447 | raIA binding protein 1 | 7.1 | ? |
| 124006 | AI147155 | Hs.270016 | ESTs | 8.3 | SS, |
| 124070 | AI950314 | Hs.154762 | HIV-1 rev binding protein 2 | 3.8 | other |
| 124074 | H05635 | Hs.294030 | topoisomerase-related function protein 4 | 1.2 | SS, |
| 124178 | BE463721 | Hs.97101 | putative G protein-coupled receptor | 32 | ? |
| 124203 | AA372796 | Hs.269339 | ESTs, Weakly similar to AF1613561 HSPC0 | 5.7 | other |
| 124352 | AA640891 | Hs.102406 | ESTs | 3.1 | TM |
| 124375 | D87454 | Hs.192966 | KIAA0265 protein | 3.5 | other |
| 124385 | AI267847 | | gb:aq49a10.x1 Stanley Frontal NB pool 2 | 57.1 | ? |
| 124390 | AA317338 | Hs.7535 | COBW-like protein | 2.8 | other |
| 124391 | AF155099 | Hs.279780 | NY-REN-18 antigen | 7.1 | other |
| 124417 | N34059 | | gb:yv28h09.s1 Soares fetal liver spleen | 3.3 | other |
| 124428 | H13540 | Hs.82202 | ribosomal protein L17 | 2.9 | other |
| 124440 | AA532519 | Hs.129043 | Human DNA sequence from clone 989H11 on | 7.9 | other |
| 124466 | R10084 | Hs.113319 | kinesin heavy chain member 2 | 2.6 | TM |
| 124462 | N53935 | | gb:yv59d0g.s1 Soares fetal liver spleen | 7.9 | TM |
| 124498 | H79433 | Hs.268997 | ESTs | 7.8 | other |
| 124515 | AA669097 | Hs.109370 | ESTs | 3.3 | other |
| 124608 | N71076 | Hs.102800 | ESTs, Weakly similar to neuronal thread | 4.6 | ? |
| 124631 | NM_014053 | Hs.270594 | FLVCR protein | 3.2 | other |
| 124634 | AI765123 | Hs.143671 | Homo sapiens cDNA FLJ13533 fis, clone PL | 5.8 | other |
| 124637 | AA160474 | Hs.75798 | hypothetical protein | 9.3 | other |
| 124642 | AW968856 | Hs.278569 | sorting nexin 17 | 3.5 | other |
| 124649 | N92593 | Hs.313054 | ESTs | 6.1 | TM |
| 124661 | R48170 | Hs.78436 | EphB1 | 5.6 | other |
| 124683 | AA381661 | Hs.119878 | ESTs, Weakly similar to M3K9_HUMAN MITOG | 7.9 | TM |
| 124712 | R09166 | Hs.191148 | ESTs | 5.7 | other |
| 124735 | R22952 | Hs.268685 | ESTs | 11.3 | ? |
| 124761 | AA374756 | Hs.93560 | Homo sapiens mRNA for KIAA1771 protein, | 9 | other |
| 124768 | AW368528 | Hs.100855 | ESTs | 8.3 | other |
| 124775 | R41772 | Hs.100878 | ESTs | 4.9 | other |
| 124777 | R41933 | Hs.140237 | ESTs, Weakly similar to ALU1_HUMAN ALU S | 2.8 | other |
| 124788 | R43543 | Hs.100912 | Homo sapiens cDNA: FLJ22726 fis, clone H | 5.1 | other |
| 124809 | AL355722 | Hs.106875 | Homo sapiens EST from clone 35214, full | 4.2 | other |
| 124811 | R46068 | Hs.288912 | hypothetical protein FLJ22604 | 14.2 | other |
| 124812 | R47948 | Hs.188732 | ESTs | 7.9 | other |
| 124822 | AA418160 | Hs.86043 | Homo sapiens cDNA FLJI3558 fis, clone PL | 6.6 | other |
| 124825 | AA501669 | Hs.336693 | ESTs | 2.3 | SS, TM |
| 124833 | AW975868 | Hs.294100 | ESTs | 2.7 | SS, TM |
| 124857 | R63652 | Hs.137190 | ESTs | 2.3 | other |
| 124860 | R65763 | Hs.101477 | EST | 23.9 | ? |
| 124863 | AI382555 | Hs.127950 | bromodomain-containing 1 | 2 | other |
| 124876 | AF135422 | Hs.27059 | GDP-mannose pyrophosphorylase A | 4.4 | SS, |
| 124878 | BE397530 | Hs.288057 | hypothetical protein FLJ22242 | 2.7 | other |
| 124902 | H37941 | Hs.101883 | ESTs | 5.7 | other |
| 124903 | AW296713 | Hs.221441 | ESTs | 32.4 | other |
| 124930 | AI076343 | Hs.173939 | ESTs, Weakly similar to ALUB_HUMAN !!!! | 22.8 | other |
| 124942 | R99978 | Hs.268892 | ESTs, Moderately similar to B34087 hypot | 6.1 | other |
| 124958 | AI078645 | Hs.431 | murine leukemia viral (bmi-1) oncogene h | 1.9 | other |
| 124980 | T40841 | Hs.98681 | ESTs | 4.5 | ? |
| 125002 | T59338 | Hs.269463 | ESTs, Weakly similar to ALU1_HUMAN ALU S | 4.9 | other |
| 125047 | T79815 | Hs.279793 | ESTs | 5 | ? |
| 125051 | T79956 | Hs.100588 | EST | 135.3 | ? |
| 125056 | T81310 | Hs.100592 | ESTs | 5.4 | other |
| 125101 | AI472068 | Hs.286236 | KIAA1856 protein | 5.6 | other |
| 125113 | T96595 | Hs.302270 | ESTs, Weakly similar to ALUF_HUMAN !!!! | 1.8 | other |
| 125115 | T97341 | | gb:ye57e05.s1 Soares fetal liver spleen | 9.6 | ? |
| 125125 | AI222382 | Hs.240767 | Human DNA sequence from clone RP1-12G14 | 1.5 | TM |
| 125147 | W38150 | | Empirically selected from AFFX single pr | 1.7 | ? |
| 125161 | W44657 | Hs.144232 | EST | 10.7 | ? |
| 125249 | AA630863 | Hs.131375 | ESTs, Moderately similar to ALUB_HUMAN ! | 1.3 | other |
| 125255 | AF098162 | Hs.118631 | timeless (Drosophila) homolog | 9.4 | other |
| 125279 | AW401809 | Hs.4779 | KIAA1150 protein | 1.5 | ? |
| 125280 | AI123705 | Hs.106932 | ESTs | 8.1 | ? |
| 125298 | AW972542 | Hs.289008 | Homo sapiens cDNA: FLJ21814 fis, clone H | 1.5 | other |
| 125660 | AW292171 | Hs.23978 | scaffold attachment factor B | 5.9 | other |
| 125827 | NM_003403 | Hs.97496 | YY1 transcription factor | 1.2 | ? |
| 125891 | U29589 | Hs.7138 | cholinergic receptor, muscarinic 3 | 6.5 | ? |
| 126005 | AW409701 | Hs.1578 | baculoviral IAP repeat-containing 5 (sur | 14.3 | ? |
| 126202 | AA157632 | Hs.272630 | vacuolar proton pump delta polypeptide | 2.5 | SS, |
| 126695 | AA643322 | Hs.172028 | a disintegrin and metalloproteinase doma | 9.1 | SS, TM |
| 127050 | AW411066 | Hs.274351 | CGI-89 protein | 17 | other |
| 127274 | AW966158 | Hs.58582 | Homo sapiens cDNA FLJ12789 fis, clone NT | 12.8 | other |
| 128355 | AW293012 | Hs.161623 | ESTs | 7.4 | SS, |
| 128493 | D87466 | Hs.240112 | KIAA0276 protein | 3.1 | TM |
| 128522 | BE173977 | Hs.10098 | putative nucleolar RNA helicase | 9.4 | other |
| 128527 | AA504583 | Hs.101047 | transcription factor3 (E2A immunoglobul | 1.5 | other |
| 128528 | R39234 | Hs.251699 | ESTs, Weakly similar to IDN4-GGTR14 [H.s | 2.8 | other |
| 128595 | U31875 | Hs.272499 | short-chain alcohol dehydrogenase family | 12.1 | TM |
| 128599 | NM_015366 | Hs.102336 | Rho GTPase activating protein 8 | 2.4 | ? |
| 128604 | AI879099 | Hs.102397 | GIOT-3 for gonadotropin inducible transc | 1.3 | other |
| 128608 | BE267994 | Hs.102419 | zinc finger protein | 7.2 | other |
| 128625 | AB037841 | Hs.102652 | hypothetical protein ASH1 | 1.3 | other |
| 128629 | AL096748 | Hs.102708 | DKFZP434A043 protein | 3.2 | other |
| 128639 | AW582962 | Hs.102897 | CGI-47 protein | 2 | TM |
| 128656 | AA458542 | Hs.10326 | coatomer protein complex, subunit epsilo | 1.4 | other |
| 128658 | BE397354 | Hs.324830 | diptheria toxin resistance protein requl | 2.5 | other |
| 128670 | AA975486 | Hs.103441 | Homo sapiens, Similar to RIKEN cDNA 1700 | 7.1 | ? |
| 128691 | W27939 | Hs.103834 | hypothetical protein MGC5576 | 7.8 | ? |
| 128696 | BE081143 | Hs.225977 | nuclear receptor coactivator 3 | 3.8 | other |
| 128700 | Y15221 | Hs.103982 | small inducible cytokine subfamily B (Cy | 1.6 | other |
| 128714 | T85231 | Hs.179661 | tubulin, beta 5 | 7.8 | other |
| 128717 | AK001564 | Hs.104222 | hypothetical protein FLJ10702 | 5.5 | other |
| 128733 | BE147740 | Hs.104558 | ESTs, Moderately similar to 138022 hypot | 2.7 | TM |
| 128737 | AF292100 | Hs.104613 | RP42 homolog | 2.8 | TM |
| 128742 | AA307211 | Hs.251531 | proteasome (prosome, macropain) subunit, | 4.5 | ? |
| 128746 | AI470163 | Hs.323342 | aclin related protein 213 complex, subun | 2.2 | other |
| 128747 | AB027249 | Hs.104741 | PDZ-binding kinase; T-cell originated pr | 2.8 | other |
| 128772 | 6E302796 | Hs.105097 | thymidine kinase 1, soluble | 5.4 | other |
| 128781 | N71826 | Hs.105465 | small nuclear ribonucleoprotein polypept | 53.9 | TM |
| 128797 | NM_002975 | Hs.105927 | stem cell growth factor; lymphocyte secr | 13.3 | other |
| 128806 | AW630942 | Hs.106061 | RD RNA-binding protein | 2.6 | other |
| 128814 | AW248431 | Hs.256526 | nuclear prelamin A recognition factor | 2.2 | other |
| 128830 | BE281170 | Hs.106357 | valosin-containing protein | 6 | other |
| 128835 | AK001731 | Hs.106390 | Homo sapiens mRNA; cDNA DKFZp586H0924 (f | 1.6 | SS, |
| 128854 | BE159181 | Hs.168232 | hypothetical protein FLJ13855 | 2.3 | other |
| 128871 | AF189723 | Hs.106778 | ATPase, Ca++ transporting, type 20, memb | 1.5 | ? |
| 128906 | R57988 | Hs.10706 | epithelial protein lost in neoplasm beta | 4.8 | other |
| 128920 | AA622037 | Hs.166468 | programmed cell death 5 | 1.4 | other |
| 128925 | R67419 | Hs.21851 | Homo sapiens cDNA FLJ12900 fis, clone NT | 1.9 | other |
| 128946 | Y13153 | Hs.107318 | kynurenine 3-monooxygenase (kynurenine 3 | 7.3 | ? |
| 128949 | AA009647 | Hs.8850 | a disintegnn and metalloproteinase doma | 2.5 | other |
| 128959 | AI580127 | Hs.107381 | hypothetical protein FLJII200 | 1.3 | other |
| 128965 | AW150697 | Hs.107418 | ESTs | 1.4 | ? |
| 128970 | AI375672 | Hs.165028 | ESTs | 1.3 | other |
| 128975 | 6E560779 | Hs.284233 | NICE-5 protein | 14 | other |
| 128979 | AW271217 | Hs.281434 | Homo sapiens cDNA FLJ14028 fis, clone HE | 1.6 | TM |
| 128995 | AI816224 | Hs.107747 | DKFZP566C243 protein | 1.9 | other |
| 129019 | AI950087 | | gbwq05c02.x1 NCI_CGAP_Kid12 Homo sapien | 2.9 | other |
| 129021 | AL044675 | Hs.173081 | KIAA0530 protein | 3.8 | other |
| 129032 | R80088 | Hs.108104 | ubiquitin-conjugating enzyme E2L 3 | 3.4 | other |
| 129076 | AW296806 | Hs.326234 | ESTs, Highly similar to T46422 hypotheti | 5 | other |
| 129078 | AI351010 | Hs.102267 | lysosomal | 2.1 | other |
| 129088 | AA744610 | Hs.194431 | palladin | 17.1 | other |
| 129095 | L12350 | Hs.108623 | thrombospondin 2 | 2.7 | other |
| 129096 | AA463189 | Hs.288906 | WW Domain-Containing Gene | 20.9 | TM |
| 129097 | BE243933 | Hs.108642 | zinc finger protein 22 (KOX 15) | 3 | other |
| 129099 | AF146074 | Hs.108660 | ATP-binding cassette, sub-family C (CFTR | 5.8 | TM |
| 129136 | W93048 | Hs.250723 | hypothetical protein MGC2747 | 6 | other |
| 129149 | AA356620 | Hs.108947 | KIAA0050 gene product | 6.4 | TM |
| 129172 | AW162916 | Hs.241576 | hypothetical protein PR02577 | 1.8 | TM |
| 129192 | AA286914 | Hs.183299 | ESTs | 2.1 | ? |
| 129194 | AA150797 | Hs.109276 | latexin protein | 3.3 | SS, TM |
| 129198 | N57532 | Hs.109315 | KIAA1415 protein | 5.9 | other |
| 129207 | AI934365 | Hs.109439 | osteoglycin (osteoinductive factor, mime | 8.1 | other |
| 129228 | U40714 | Hs.239307 | tyrosyl-tRNA synthetase | 2.9 | other |
| 129229 | AF013758 | Hs.109643 | polyadenylate binding protein-interactin | 3.3 | ? |
| 129254 | AA252468 | Hs.1098 | DKFZp434J1813 protein | 2.6 | SS, TM |
| 129255 | AI961727 | Hs.109804 | H1 histone family, member X | 7.4 | other |
| 129288 | W26392 | Hs.110080 | ESTs, Weakly similar to S13495 pregnancy | 9.6 | other |
| 129296 | AI051967 | Hs.110122 | ESTs | 1.2 | other |
| 129323 | AA287239 | Hs.5518 | Homo sapiens cDNA FLJ11311 fis, clone PL | 5.2 | other |
| 129340 | H75334 | Hs.11050 | F-box only protein 9 | 4.7 | SS, |
| 129347 | BE614192 | Hs.279869 | melanoma-associated antigen recognised b | 7.7 | TM |
| 129362 | U30246 | Hs.110736 | solute carrier family 12 (sodium/potassi | 6.7 | TM |
| 129366 | BE220806 | Hs.184697 | Homo sapiens clone 23785 mRNA sequence | 8.6 | SS, |
| 129370 | AI686379 | Hs.110796 | SAR1 protein | 1.4 | TM |
| 129372 | NM_016039 | Hs.110803 | CGI-99 protein | 2 | other |
| 129403 | AF149785 | Hs.111126 | pituitary tumor-transforming 1 interacti | 7.5 | other |
| 129404 | AI267700 | Hs.317584 | ESTs | 5.1 | other |
| 129423 | AA204686 | Hs.234149 | hypothetical protein FLJ20547 | 10.2 | other |
| 129482 | AA188185 | Hs.289043 | spindlin | 6.8 | other |
| 129513 | AW843633 | Hs.306163 | hypothetical protein AL110115 | 7.1 | SS, |
| 129515 | AF255303 | Hs.112227 | membrane-associated nucleic acid binding | 2.5 | other |
| 129527 | AA769221 | Hs.270847 | delta-tubulin | 3.2 | other |
| 129559 | W01296 | Hs.11360 | hypothetical protein FLJ14784 | 7.5 | other |
| 129560 | AA317841 | Hs.7845 | hypothetical protein MGC2752 | 6.8 | other |
| 129570 | AI923097 | Hs.11441 | chromosome 1 open reading frame 8 | 2.1 | other |
| 129575 | F08282 | Hs.278428 | progestin induced protein | 1.6 | other |
| 129587 | H14718 | Hs.11506 | Human clone 23589 mRNA sequence | 6.8 | other |
| 129588 | BE408300 | Hs.301862 | postmeiotic segregation increased 2-like | 1.4 | TM |
| 129591 | N57423 | Hs.179898 | HSPC055 protein | 7.4 | other |
| 129594 | AW403724 | Hs.36989 | coagulation factor VII (serum prothrombi | 9 | ? |
| 129596 | AF035537 | Hs.115521 | REV3 (yeast homolog)-like, catalytic sub | 1.6 | other |
| 129628 | U38945 | Hs.1174 | cyclin-dependent kinase inhibitor 2A (me | 2.2 | other |
| 129649 | AD000092 | Hs.16488 | calreticulin | 3.3 | other |
| 129675 | NM_015556 | Hs.172180 | KIAA0440 protein | 13.4 | other |
| 129680 | U03749 | | gb:Human chromogranin A (CHGA) gene, pro | 14.1 | ? |
| 129689 | AW748482 | Hs.77873 | B7 homolog 3 | 2.6 | other |
| 129702 | AI304966 | Hs.12035 | ESTs, Weakly similar to 138022 hypotheti | 7.5 | TM |
| 129720 | AA156214 | Hs.12152 | APMCF1 protein | 2 | other |
| 129721 | NM_001415 | Hs.211539 | eukaryotic translation initiation factor | 1.7 | TM |
| 129778 | AK001676 | Hs.12457 | hypothetical protein FLJ10814 | 1.8 | other |
| 129779 | AA394090 | Hs.12460 | Homo sapiens done 23870 mRNA sequence | 5.5 | TM |
| 129800 | AF052112 | Hs.12540 | lysosomal | 1.7 | ? |
| 129806 | AB023148 | Hs.173373 | KIAA093I protein | 1.2 | other |
| 129815 | BE565817 | Hs.26498 | hypothetical protein FLJ21657 | 3.1 | other |
| 129840 | NM_006590 | Hs.12820 | SnRNP assembly defective 1 homolog | 1.8 | other |
| 129861 | AL049999 | Hs.85963 | DKFZP564M182 protein | 2.3 | other |
| 129864 | AI393237 | Hs.129914 | runt-related transcription factor 1 (acu | 1.7 | SS, |
| 129869 | AI222069 | Hs.13015 | hypothetical protein similar to mouse Dn | 2.8 | TM |
| 129945 | BE514376 | Hs.165998 | PAI-1 mRNA-binding protein | 1.8 | other |
| 129953 | AA412195 | Hs.13740 | ESTs | 2.5 | other |
| 129972 | AW753185 | Hs.180628 | dynamin 1-like | 1.8 | ? |
| 129983 | U09848 | Hs.132390 | zinc finger protein 36 (KOX 18) | 1.3 | other |
| 130010 | AA301116 | Hs.142838 | nucleolar phosphoprotein Nopp34 | 1.6 | other |
| 130081 | AA287325 | Hs.14713 | ESTs | 4.1 | other |
| 130082 | S73265 | Hs.1473 | gastrin-releasing peptide | 1.9 | other |
| 130097 | AL048962 | Hs.14845 | forkhead box O3A | 2.8 | other |
| 130100 | AL135561 | Hs.14891 | hypothetical protein FLJ21047 | 2.3 | other |
| 130111 | X53002 | Hs.149846 | integrin, beta5 | 2.3 | other |
| 130112 | AA916785 | Hs.180610 | splicing factor proline/glutamine rich ( | 3 | other |
| 130128 | L76937 | Hs.150477 | Wemer syndrome | 1.8 | other |
| 130135 | AA311426 | Hs.21635 | tubulin, gamma | 16.1 | other |
| 130211 | NM003358 | Hs.23703 | ESTs, Moderately similar to CEGT_HUMAN C | 1.6 | other |
| 130212 | D80001 | Hs.152629 | KIAA0179 protein | 1.3 | other |
| 130236 | R85367 | Hs.51957 | splicing factor, arginine/serine-rich 2, | 2 | other |
| 130241 | AL035588 | Hs.153203 | MyoD family inhibitor | 3.2 | other |
| 130242 | X79201 | Hs.153221 | synovial sarcoma, translocated to X chro | 5.4 | ? |
| 130249 | D81983 | Hs.322852 | GAS2-related on chromosome 22 | 4.9 | other |
| 130263 | NM_002497 | Hs.153704 | NIMA (never in mitosis gene a)-related k | 1.4 | other |
| 130287 | AA479005 | Hs.154036 | tumor suppressing subtransferable candid | 2.6 | other |
| 130310 | ABOII121 | Hs.154248 | amyotrophic lateral sclerosis 2 (juvenil | 6.3 | other |
| 130353 | Z19084 | Hs.172210 | MUF1 protein | 6.2 | other |
| 130356 | AF127577 | Hs.155017 | nuclear receptor interacting protein 1 | 2.4 | other |
| 130357 | AJ224442 | Hs.155020 | putative methyltransferase | 3.5 | TM |
| 130359 | NM_013449 | Hs.277401 | bromodomain adjacent to zinc finger doma | 8.5 | other |
| 130367 | AL135301 | Hs.8768 | hypothetical protein FU10849 | 1.4 | other |
| 130372 | AI077464 | Hs.5011 | RNA binding motif protein 9 | 3.3 | ? |
| 130393 | N89487 | Hs.155291 | KIAA0005 gene product | 1.8 | other |
| 130399 | AW374106 | Hs.155356 | hypothetical protein MGC2840 similar to | 3.4 | other |
| 130407 | BE385099 | Hs.334727 | hypothetical protein MG03017 | 2.3 | other |
| 130409 | NM_001197 | Hs.155419 | BCL2-interacting killer (apoptosis-induc | 2.7 | TM |
| 130419 | AF037448 | Hs.155489 | NS1-associated protein 1 | 1.8 | other |
| 130441 | U63630 | Hs.155637 | protein kinase, DNA-activated, catalytic | 2.3 | other |
| 130448 | BE513202 | Hs.15589 | PPAR binding protein | 4 | TM |
| 130455 | D90041 | Hs.155956 | N-acetyltransferase 1 (arylamine N-acety | 33.6 | ? |
| 130485 | BE245851 | Hs.180779 | H2B histone family, member B | 5 | other |
| 130487 | U49844 | Hs.77613 | ataxia telangiectasia and Rad3 related | 4.4 | other |
| 130498 | L38951 | Hs.180446 | karyopherin (importin) beta 1 | 1.6 | SS, TM |
| 130503 | BE208491 | Hs.295112 | KIAA0618 gene product | 16.1 | other |
| 130511 | L32137 | Hs.1584 | cartilage oligomeric matrix protein (pse | 6.1 | other |
| 130526 | AW876523 | Hs.15929 | hypothetical protein FLJ12910 | 2.1 | other |
| 130544 | AA321238 | Hs.4310 | eukaryotic translation initiation factor | 1.5 | other |
| 130553 | AF062649 | Hs.252587 | pituitary tumor-transforming 1 | 14.4 | ? |
| 130556 | AI907018 | Hs.15977 | Empirically selected from AFFX single pr | 4.8 | other |
| 130567 | AA383092 | Hs.1608 | replication protein A3 (14kD) | 8 | other |
| 130568 | AA232119 | Hs.16085 | putative G-protein coupled receptor | 3.4 | other |
| 130574 | AF083208 | Hs.16178 | apoptosis antagonizing transcription fac | 1.2 | other |
| 130598 | AL042210 | Hs.16493 | hypothetical protein DKFZp762N2316; KIAA | 1.4 | other |
| 130601 | AA609738 | Hs.16525 | ESTs | 1.5 | TM |
| 130614 | AI354355 | Hs.16697 | down-regulator of transcription 1, TBP-b | 1.3 | other |
| 130617 | M90516 | Hs.1674 | glutamine-fructose-6-phosphate transamin | 12.1 | TM |
| 130618 | AA383439 | Hs.16758 | Spir-1 protein | 15.9 | other |
| 130667 | BE246961 | Hs.17639 | Homo sapiens ubiquitin protein ligase (U | 13.9 | other |
| 130674 | AL048842 | Hs.194019 | atlractin | 1.5 | other |
| 130675 | AA442233 | Hs.17731 | hypothetical protein FLJ12892 | 5.4 | other |
| 130692 | AA652501 | Hs.13561 | hypothetical protein MGC4692 | 5 | other |
| 130693 | R68537 | Hs.17962 | ESTs | 2 | other |
| 130712 | AJ271881 | Hs.279762 | bromodomain-containing 7 | 1.8 | TM |
| 130714 | AI348274 | Hs.18212 | DNA segment on chromosome X (unique) 987 | 2 | TM |
| 130730 | AB007920 | Hs.18586 | KIAA0451 gene product | 3.8 | ? |
| 130744 | H59696 | Hs.18747 | POP7 (processing of precursor, S. cerevi | 3.2 | ? |
| 130751 | AF052105 | Hs.18879 | chromosome 12 open reading frame | 1.4 | other |
| 130757 | AL036067 | Hs.18925 | protein x 0001 | 5.7 | other |
| 130768 | AF258627 | Hs.211562 | ATP-binding cassette, sub-family A (ABC1 | 5.2 | ? |
| 130789 | AK000355 | Hs.8899 | sirtuin (silent mating type information | 1.6 | other |
| 130836 | J05068 | Hs.2012 | transcobalamin I (vitamin B12 binding pr | 15.7 | SS, |
| 130841 | AL157468 | Hs.325825 | Homo sapiens cDNA FLJ20848 fis, clone AD | 2.8 | other |
| 130843 | AA447492 | Hs.20183 | ESTs, Weakly similar to AF164793 1 prote | 1.5 | other |
| 130844 | U76248 | Hs.20191 | seven in absentia (Drosophila) homolog 2 | 3.5 | other |
| 130855 | AJ243706 | Hs.143323 | putative DNA/chromatin binding motif | 1.7 | other |
| 130861 | NM_016578 | Hs.20509 | HBV pX associated protein-8 | 1.9 | other |
| 130879 | NM_003416 | Hs.2076 | zinc finger protein 7 (KOX 4, clone HF.1 | 1.4 | other |
| 130880 | BE514434 | Hs.20830 | kinesin-like 2 | 2.1 | TM |
| 130892 | AL120837 | Hs.20993 | high-glucose-regulated protein 8 | 2.5 | other |
| 130898 | AB033078 | Hs.186613 | sphingosine-1-phosphate lyase 1 | 1.7 | other |
| 130911 | BE409769 | Hs.21189 | DnaJ (Hsp40) homolog, subfamily A, membe | 1.8 | other |
| 130919 | N79110 | Hs.21276 | collagen, type IV, alpha 3 (Goodpasture | 2.3 | TM |
| 130944 | BE382657 | Hs.21486 | signal transducer and activator of trans | 5.4 | other |
| 130971 | N39842 | Hs.301444 | KIAA1673 | 2.2 | SS, |
| 130993 | T97401 | Hs.21929 | ESTs | 1.6 | other |
| 131005 | AV658308 | Hs.2210 | thyroid hormone receptor interactor 3 | 1.6 | ? |
| 131028 | AI879165 | Hs.2227 | CCAAT/enhancer binding protein (C/EBP), | 1.2 | other |
| 131042 | AI826288 | Hs.171637 | hypothetical protein MGC2628 | 1.6 | other |
| 131046 | AA321649 | Hs.2248 | small inducible cytokine subfamily B (Cy | 7.4 | ? |
| 131060 | AA194422 | Hs.22564 | myosin VI | 5.1 | other |
| 131070 | N53344 | Hs.22607 | ESTs | 7.1 | other |
| 131076 | AA749230 | Hs.26433 | dolichyl-phosphate (UDP-N-acetylglucosam | 2.1 | TM |
| 131099 | AL133353 | Hs.226581 | COX15 (yeast) homolog, cytochrome c oxid | 7.1 | other |
| 131174 | NM_006540 | Hs.29131 | nuclear receptor coactivator 2 | 1.9 | ? |
| 131185 | BE280074 | Hs.23960 | cyclin B1 | 5.8 | ? |
| 131206 | AW138839 | Hs.24210 | ESTs | 2 | other |
| 131213 | M885699 | Hs.24332 | CGI-26 protein | 7.1 | TM |
| 131225 | H62087 | Hs.31659 | thyroid hormone receptor-associated prot | 7.6 | ? |
| 131231 | N47468 | Hs.59757 | zinc finger protein 281 | 2.9 | other |
| 131233 | D89053 | Hs.268012 | fatty-acid-Coenzyme A ligase, long-chain | 3.5 | other |
| 131243 | AW383256 | Hs.24752 | spectrmn SH3 domain binding protein 1 | 2.8 | ? |
| 131246 | AL080080 | Hs.24766 | thioredoxin domain-containing | 2.8 | SS, TM |
| 131247 | AL043100 | Hs.326190 | fatty acid amide hydrolase | 5.6 | other |
| 131281 | AA251716 | Hs.25227 | ESTs | 5.8 | other |
| 131283 | X80038 | Hs.339713 | Homo sapiens clone F19374 APO E-C2 gene | 1.3 | other |
| 131305 | AV656017 | Hs.184325 | CGI-76 protein | 5 | ? |
| 131320 | AA505691 | Hs.145696 | splicing factor (CC1.3) | 1.8 | TM |
| 131339 | AF058696 | Hs.25812 | Nijmegen breakage syndrome 1 (nibrin) | 2.6 | other |
| 131375 | AW293165 | Hs.143134 | ESTs | 5.4 | other |
| 131390 | BE269388 | Hs.182698 | mitochondrial ribosomal protein L20 | 5.3 | other |
| 131410 | BE259110 | Hs.279836 | HSPC166 protein | 2.2 | other |
| 131412 | NM_012247 | Hs.124027 | SELENOPHOSPHATE SYNTHETASE; Human selen | 2 | ? |
| 131429 | AL046302 | Hs.26750 | hypothetical protein FLJ21908 | 1.4 | other |
| 131458 | BE297567 | Hs.27047 | hypothetical protein FLJ20392 | 1.7 | other |
| 131475 | AA992841 | Hs.27263 | KIAA1458 protein | 2 | other |
| 131501 | AV661958 | Hs.8207 | GK001 protein | 2.6 | other |
| 131511 | AA732153 | Hs.27865 | Homo sapiens cDNA: FLJ21333 fis, clone C | 2 | other |
| 131528 | AU076408 | Hs.28309 | UDP-glucose dehydrogenase | 1.6 | TM |
| 131532 | BE268278 | Hs.28393 | hypothetical protein MGC2592 | 7.4 | other |
| 131543 | AW966881 | Hs.41639 | programmed cell death 2 | 2.2 | other |
| 131544 | AL355715 | Hs.28555 | programmed cell death 9 (PDCD9) | 2.1 | other |
| 131562 | NM_003512 | Hs.28777 | H2A histone family, member L | 1.7 | other |
| 131564 | T93500 | Hs.28792 | Homo sapiens cDNA FLJ11041 fis, clone PL | 5.2 | other |
| 131569 | AL389951 | Hs.271623 | nudeoporin 50kD | 5 | other |
| 131618 | BE393822 | Hs.29645 | Homo sapiens mRNA; cDNA DKFZp7610029 (fr | 1.8 | other |
| 131622 | R78195 | Hs.29692 | Homo sapiens cDNA FLJ11436 fis, clone HE | 1.3 | other |
| 131623 | AB037791 | Hs.29716 | hypothetical protein FLJ10980 | 2.2 | TM |
| 131643 | AW410601 | Hs.30026 | HSPC182 protein | 3 | other |
| 131653 | AW960597 | Hs.30164 | ESTs | 1.3 | other |
| 131656 | AI218918 | Hs.30209 | KIAA0854 protein | 2.8 | other |
| 131669 | X52486 | Hs.3041 | uracil-DNA glycosylase 2 | 2.8 | other |
| 131692 | BE559681 | Hs.30736 | KIAA0124 protein | 5.6 | ? |
| 131714 | AA642831 | Hs.31016 | putative DNA binding protein | 2.9 | ? |
| 131722 | D13757 | Hs.311 | phosphoribosyl pyrophosphate amidotransf | 3.4 | other |
| 131737 | AK001641 | Hs.31323 | inhibitor of kappa light polypeptidegen | 3.9 | ? |
| 131763 | AI878932 | Hs.317 | topoisomerase (DNA) I | 3.4 | other |
| 131772 | AA382590 | Hs.170980 | KIAA0948 protein | 25.5 | other |
| 131787 | 087077 | Hs.196275 | KIAA0240 protein | 2.4 | SS, |
| 131793 | AW966127 | Hs.32246 | Homo sapiens cDNA FLJ14656 fis, clone NT | 8 | TM |
| 131795 | BE501849 | Hs.32317 | high-mobility group 208 | 1.5 | other |
| 131798 | X86098 | Hs.301449 | adenovirus 5 EIA binding protein | 4.2 | other |
| 131817 | U20536 | Hs.3280 | caspase 6, apoptosis-related cysteine pr | 4.3 | other |
| 131824 | U28838 | Hs.32935 | TATA box binding protein (TBP)-associate | 3.5 | other |
| 131850 | AI251317 | Hs.33184 | ESTs | 5.2 | TM |
| 131878 | AA083764 | Hs.6101 | hypothetical protein MGC3178 | 5.9 | other |
| 131885 | BE502341 | Hs.3402 | ESTs | 13.7 | other |
| 131900 | AA099014 | Hs.231029 | Homo sapiens, clone MGC:15961, mRNA, com | 8.7 | other |
| 131904 | AF078866 | Hs.284296 | Homo sapiens cDNA: FLJ22993 fis, clone K | 5.5 | other |
| 131905 | AA179298 | Hs.3439 | stomatin-like 2 | 11.3 | other |
| 131913 | AW207440 | Hs.185973 | degenerative spermatocyte (homolog Droso | 1.7 | SS, |
| 131916 | AA025976 | Hs.34569 | ESTs | 5.2 | TM |
| 131925 | AF151048 | Hs.183180 | anaphase promoting complex subunit 11 (y | 2.8 | other |
| 131929 | BE541211 | Hs.34804 | Homo sapiens cDNA FLJ11472 fis, clone HE | 5.4 | TM |
| 131941 | BE252983 | Hs.35086 | ubiquitin specific protease 1 | 2.4 | other |
| 131950 | AA355113 | Hs.35380 | x 001 protein | 1.5 | ? |
| 131962 | AK000046 | Hs.267448 | hypothetical protein FLJ20039 | 2.3 | other |
| 131965 | W79283 | Hs.35962 | ESTs | 1.4 | other |
| 131971 | BE567100 | Hs.154938 | hypothetical protein MDS025 | 3.5 | other |
| 131977 | U90441 | Hs.3622 | procollagen-proline, 2-oxoglutarate 4-di | 6.6 | TM |
| 131985 | AA503020 | Hs.36563 | hypothetical protein FLJ22418 | 2.4 | ? |
| 131991 | AF053306 | Hs.36708 | budding uninhibited by benzimidazoles 1 | 2.2 | SS, TM |
| 132019 | H56995 | Hs.37372 | Homo sapiens DNA binding peptide mRNA, p | 3.3 | TM |
| 132062 | BE266155 | Hs.3832 | clathrin-associated protein AP47 | 1.5 | other |
| 132084 | NM_902267 | Hs.3886 | karyaphenn alpha 3 (importin alpha 4) | 3.7 | other |
| 132103 | BE171921 | Hs.3991 | ESTs | 1.5 | other |
| 132105 | AV646076 | Hs.39959 | ESTs | 5.8 | TM |
| 132116 | AW960474 | Hs.40289 | ESTs | 1.7 | other |
| 132176 | AA857025 | Hs.8878 | kinesin-like 1 | 3.4 | other |
| 132180 | NM_004460 | Hs.418 | fibroblast activation protein, alpha | 14.7 | SS, |
| 132194 | R42432 | Hs.4212 | ESTs | 2.2 | other |
| 132207 | BE206939 | Hs.42287 | E2F transcription factor 6 | 1.5 | other |
| 132235 | AV658411 | Hs.42656 | KIAA1681 protein | 5.7 | other |
| 132252 | AI566004 | Hs.141269 | Homo sapiens cDNA: FLJ21550 fis, clone C | 2.1 | other |
| 132266 | AA301228 | Hs.43299 | hypothetical protein FLJ12890 | 1.5 | other |
| 132273 | AA227710 | Hs.43658 | DKFZP586L151 protein | 10 | other |
| 132276 | AA653507 | Hs.285711 | hypothetical protein FLJ13089 | 2 | other |
| 132288 | N36110 | Hs.305971 | solute canier family 2 (facilitated glu | 9.2 | other |
| 132294 | A8023191 | Hs.44131 | KIAA0974 protein | 2 | other |
| 132298 | NM_015986 | Hs.7120 | cytokine receptor-like molecule 9 | 6.6 | SS, |
| 132299 | AW405882 | Hs.44205 | cortistatin | 3.8 | other |
| 132325 | N37065 | Hs.44856 | hypothetical protein FLJI2116 | 1.5 | other |
| 132370 | AW572805 | Hs.46645 | ESTs | 28.3 | ? |
| 132374 | AF155582 | Hs.46744 | core1 UDP-galactose:N-acetylgalactosamin | 1.9 | other |
| 132376 | AI279892 | Hs.46801 | sorting nexin 14 | 2 | ? |
| 132384 | AA312135 | Hs.46967 | HSPCO34 protein | 6.1 | ? |
| 132393 | AL135094 | Hs.47334 | hypothetical protein FLJ14495 | 1.7 | other |
| 132450 | AA100012 | Hs.48827 | hypothetical protein FLJ12085 | 8.6 | other |
| 132452 | AW973521 | Hs.247324 | mitochondrial ribosomal protein S14 | 5.3 | other |
| 132456 | A2011084 | Hs.48924 | KIAA0512 gene product; ALEX2 | 1.5 | other |
| 132470 | AI224456 | Hs.4934 | H. sapiens polyA site DNA | 2 | other |
| 132484 | X16660 | Hs.119007 | RAB4, member RAS oncogene family | 2.9 | SS, |
| 132518 | AW885606 | Hs.5064 | ESTs | 2.2 | other |
| 132530 | AA306105 | Hs.50785 | SEC22, vesicle trafficking protein (S.c | 1.7 | other |
| 132532 | AA454132 | Hs.5080 | mitochondrial ribosomal protein L16 | 7.2 | TM |
| 132534 | BE388673 | Hs.5086 | hypothetical protein MG010433 | 2.2 | SS, |
| 132543 | BE568452 | Hs.5101 | protein regulator of cytokinesis 1 | 2.2 | other |
| 132574 | AW631437 | Hs.5184 | TH1 drosophila homolog | 14 | ? |
| 132596 | AK001484 | Hs.5298 | CGI-45 protein | 1.9 | other |
| 132611 | AA345547 | Hs.53263 | hypothetical protein FLJ13287 | 2.6 | TM |
| 132612 | H12751 | Hs.5327 | PRO1914 protein | 2 | other |
| 132616 | 8E262677 | Hs.283558 | hypothetical protein PR01855 | 3.1 | other |
| 132638 | AI796870 | Hs.54277 | DNA segment on chromosome X (unique) 992 | 12.4 | TM |
| 132668 | A8018319 | Hs.5460 | KIAA0776 protein | 2.8 | SS, |
| 132692 | AW191962 | Hs.249239 | collagen, type VIII, alpha 2 | 3 | other |
| 132715 | F11875 | Hs.5534 | Homo sapiens cDNA FLJ12961 fis, clone NT | 1.8 | other |
| 132718 | NMSO4600 | Hs.554 | Sjogren syndrome antigen A2 (60kD, ribon | 3.7 | other |
| 132724 | AI142265 | Hs.55498 | geranylgeranyl diphosphate synthase 1 | 1.8 | TM |
| 132731 | AI189075 | Hs.301872 | hypothetical protein MGC4840 | 5.9 | other |
| 132744 | AA010233 | Hs.55921 | glutamyl-prolyl-tRNA synthetase | 8.7 | other |
| 132760 | AA125985 | Hs.56145 | thymosin, beta, identified in neuroblast | 3.6 | other |
| 132771 | Y10275 | Hs.56407 | phosphoserine phosphatase | 2.8 | TM |
| 132773 | AA459713 | Hs.295901 | KIAA0493 protein | 14.6 | other |
| 132784 | AI142133 | Hs.56845 | GDP dissociation inhibitor 2 | 1.7 | other |
| 132798 | AI026701 | Hs.5716 | KIAA0310 gene product | 2.5 | other |
| 132807 | U07418 | Hs.57301 | mutL (E. coli) homolog 1 (colon cancer, | 1.4 | other |
| 132810 | AB007944 | Hs.5737 | KIAA0475 gene product | 4.3 | SS |
| 132813 | BE313625 | Hs.57435 | solute canier family 11 (proton-coupled | 2.8 | other |
| 132815 | AI815189 | Hs.57475 | sex comb on midleg homolog 1 | 1.6 | other |
| 132817 | N27852 | Hs.57553 | tousled-like kinase 2 | 1.4 | other |
| 132821 | AJ251595 | Hs.169610 | CD44 antigen (homing function and Indian | 5.4 | other |
| 132833 | U78525 | Hs.57783 | eukaryotic translation initiation factor | 6.1 | ? |
| 132842 | NM_16154 | Hs.279771 | Homo sapiens clone PP1596 unknown mRNA | 7.2 | other |
| 132844 | F12200 | Hs.5811 | chromosome 21 open reading frame 59 | 2.9 | other |
| 132851 | U09716 | Hs.287912 | lectin, mannose-binding, 1 | 6.1 | other |
| 132869 | AW963217 | Hs.203961 | ESTs, Moderately similar to AF116721 89 | 1.8 | other |
| 132873 | AW007683 | Hs.58598 | KIAA1266 protein | 2.2 | other |
| 132875 | NM_004850 | Hs.58617 | Rho-associated, coiled-coil containing p | 5 | TM |
| 132891 | BE267143 | Hs.59271 | U2(RNU2) small nuclear RNA auxillary fac | 2.7 | ? |
| 132897 | AW503667 | Hs.59545 | ring finger protein 15 | 5.4 | ? |
| 132902 | AI936442 | Hs.59838 | hypothetical protein FLJ10808 | 3.2 | other |
| 132912 | AW732760 | Hs.167578 | Homo sapiens cDNA FLJ11095 fis, clone PL | 1.4 | other |
| 132913 | W78714 | Hs.60257 | Homo sapiens cDNA FLJ13598 fis, clone PL | 3 | other |
| 132940 | T79136 | Hs.127243 | Homo sapiens mRNA for KIAA1724 protein, | 10.3 | other |
| 132942 | AA554458 | Hs.197751 | KIAA0666 protein | 2.1 | SS, |
| 132952 | AI658580 | Hs.61426 | Homo sapiens mesenchymal stem coil prote | 1.3 | other |
| 132962 | AA576635 | Hs.6153 | CGI-48 protein | 4.9 | other |
| 132972 | AA034365 | Hs.288924 | Homo sapiens cDNA FLJ11392 fis, clone HE | 3.6 | TM |
| 132973 | AA035446 | Hs.323277 | ESTs | 13.1 | other |
| 132977 | AA093322 | Hs.301404 | RNA binding motif protein 3 | 1.3 | other |
| 132980 | AA040696 | Hs.62016 | ESTs | 2.3 | ? |
| 132994 | M112748 | Hs.279905 | clone HQ0310 PRO0310p1 | 17.1 | other |
| 133012 | AA847843 | Hs.62711 | Homo sapiens, clone IMAGE:3351295, mRNA | 1.9 | other |
| 133015 | AJ002744 | Hs.246315 | UDP-N-acetyl-alpha-D-galactosamine:polyp | 5 | TM |
| 133062 | AW500374 | Hs.64056 | PRO0149 protein | 6.1 | other |
| 133069 | BE247441 | Hs.6430 | protein with polyglutamine repeat; calci | 1.5 | TM |
| 133091 | AK001628 | Hs.64691 | KIAA0483 protein | 1.4 | other |
| 133110 | AA808177 | Hs.65228 | ESTs | 5.6 | other |
| 133134 | AF198620 | Hs.65648 | RNA binding motif protein 8A | 1.9 | other |
| 133145 | H94227 | Hs.6592 | Homo sapiens, clone IMAGE:2961368, mRNA, | 4.8 | ? |
| 133152 | Z11695 | Hs.324473 | mitogen-activated protein kinase 1 | 5 | other |
| 133174 | AA431620 | Hs.324178 | hypothetical protein MGC2745 | 2.7 | other |
| 133175 | AW955632 | Hs.66666 | ESTs, Weakly similar to S19560 proline-r | 9.3 | other |
| 133177 | X97795 | Hs.66718 | RA054 (S.cerevisiae)-like | 4.5 | TM |
| 133208 | AI801777 | Hs.6774 | ESTs | 5.5 | TM |
| 133226 | AW954569 | Hs.296287 | Homo sapiens, Similar to bromodomain-con | 2.7 | other |
| 133228 | AI492924 | Hs.6831 | golgi phosphoprotein 1 | 1.7 | ? |
| 133254 | AI567421 | Hs.273330 | Homo sapiens, clone IMAGE:3544662, mRNA, | 1.3 | other |
| 133268 | AW956781 | Hs.293937 | ESTs, Weakly similar to FXD2_HUMAN FORKH | 12.2 | other |
| 133291 | BE297855 | Hs.69855 | NRAS-related gene | 1.2 | other |
| 133314 | AA102670 | Hs.70725 | gamma-aminobutyric acid (GABA) A recepto | 1.7 | TM |
| 133321 | 179526 | Hs.179516 | integral type I protein | 11.1 | ? |
| 133327 | AL390127 | Hs.7104 | Kruppel-like factor 13 | 2.9 | other |
| 133347 | BE257758 | Hs.71475 | acid cluster protein 33 | 2.5 | ? |
| 133360 | AI016521 | Hs.71816 | v-akt murine thymoma viral oncogene homo | 1.5 | other |
| 133366 | AA292811 | Hs.72050 | non-metastatic cells 5, protein expresse | 2.1 | other |
| 133367 | AF231919 | Hs.18759 | KIAA0539 gene product | 1.3 | other |
| 133370 | AF245505 | Hs.72157 | DKFZP56411922 protein | 2.2 | other |
| 133390 | AI950382 | Hs.72660 | phosphatidylserine receptor | 5.7 | TM |
| 133391 | AW103364 | Hs.727 | inhibin, beta A (activin A, activin AB a | 25.5 | other |
| 133394 | AA305127 | Hs.237225 | hypothetical protein HT023 | 3.3 | other |
| 133437 | AL031591 | Hs.7370 | phosphotidylinositol transfer protein, b | 1.6 | other |
| 133452 | NM 002759 | Hs.274382 | protein kinase, interferon-inducible dou | 4.1 | other |
| 133453 | AI659306 | Hs.73826 | protein tyrosine phosphatase, non-recept | 1.5 | other |
| 133500 | AW964804 | Hs.74280 | hypothetical protein FLJ22237 | 6.3 | TM |
| 133529 | W45623 | Hs.74571 | ADP-ribosylation factor 1 | 4 | ? |
| 133543 | AU077073 | Hs.108327 | damage-specific DNA binding protein 1(1 | 1.8 | ? |
| 133578 | AU077050 | Hs.75066 | translin | 1.5 | other |
| 133579 | X75346 | Hs.75074 | mitogen-activated protein kinase-activat | 3.5 | TM |
| 133582 | BE391579 | Hs.75087 | Fas-activated serine/threonine kinase | 6.8 | TM |
| 133594 | AW160781 | Hs.172589 | nuclear phosphoprotein similar to S. cer | 2.6 | TM |
| 133595 | AA393273 | Hs.75133 | transcription factor 6-like 1 (mitochond | 1.4 | other |
| 133599 | NM_002885 | Hs.75151 | RAP1, GTPase activating protein 1 | 8.1 | other |
| 133621 | NM_004893 | Hs.75258 | H2A histone family, member Y | 13.5 | other |
| 133627 | NM_002047 | Hs.75280 | glycyl-tRNA synthetase | 2.2 | other |
| 133631 | NM_000401 | Hs.75334 | exostoses (multiple) 2 | 1.8 | other |
| 133649 | U25849 | Hs.75393 | acid phosphatase 1, soluble | 2 | other |
| 133690 | AV661 185 | Hs.75574 | mitochondrial ribosomal protein L19 | 2.8 | other |
| 133720 | L27841 | Hs.75737 | pericentriolar material 1 | 6.8 | other |
| 133722 | AW969976 | Hs.279009 | matrix GIa protein | 2.5 | other |
| 133751 | AW40204&comp | Hs.334787 | Homo sapiens, Similar to likely ortholog | 3.1 | TM |
| 133757 | T52946 | Hs.196209 | RAE1 (RNA export 1, S.pombe) homolog | 1.4 | ? |
| 133760 | BE271766 | Hs.181357 | laminin receptor 1 (67kD, ribosomal prot | 5.4 | other |
| 133765 | M62194 | Hs.75929 | cadherin 11, type 2, OB-cadherin (osteob | 5 | other |
| 133780 | AA557660 | Hs.76152 | decorin | 3.8 | other |
| 133797 | AL133921 | Hs.76272 | retinoblastoma-binding protein 2 | 3.1 | ? |
| 133822 | D50525 | Hs.699 | peptidylprolyl isomerase B (cyclophilin | 9.7 | ? |
| 133842 | AW797468 | Hs.285013 | putative human HLA class II associated p | 2.4 | other |
| 133845 | AA147026 | Hs.76704 | ESTs | 2.5 | other |
| 133865 | AB011155 | Hs.170290 | discs, large (Drosophila) homolog 5 | 5 | other |
| 133867 | AW340125 | Hs.76989 | KIAA0097 gene product | 2.5 | ? |
| 133868 | AB012193 | Hs.183874 | cullin 4A | 2.1 | other |
| 133922 | U30825 | Hs.77608 | splicing factor, arginine/serine-rich 9 | 2.8 | TM |
| 133924 | D86326 | Hs.325948 | vesicle docking protein p115 | 1.8 | SS |
| 133929 | NM_006306 | Hs.211602 | SMC1 (structural maintenance of chromoso | 2 | ? |
| 133936 | L17128 | Hs.77719 | gamma-glutamyl carboxylase | 2.6 | other |
| 133941 | BE244332 | Hs.77770 | adaptor-related protein complex 3, mu 2 | 2.9 | other |
| 133959 | X81789 | Hs.77897 | splicing factor 3a, subunit 3, 60kD | 10.4 | other |
| 133976 | AI908165 | Hs.169946 | GATA-binding protein 3 (T-cell receptor | 1.9 | other |
| 133989 | AL040328 | Hs.78202 | SWI/SNF related, matrix associated, acti | 2.6 | SS, |
| 133997 | AI824113 | Hs.78281 | regulator of G-protein signalling 12 | 13 | other |
| 134010 | AB016092 | Hs.197114 | RNA binding protein: AT-rich element bin | 8.8 | other |
| 134015 | D31764 | Hs.278569 | sorting nexin 17 | 1.5 | SS, |
| 134070 | NM_003590 | Hs.78946 | cullin 3 | 8.3 | other |
| 134110 | U41060 | Hs.79136 | LIV-1 protein, estrogen regulated | 2.7 | other |
| 134129 | NM_014742 | Hs.79305 | KIAA0255 gene product | 4.2 | other |
| 134134 | H86504 | Hs.173328 | protein phosphatase 2, regulatory subuni | 1.7 | other |
| 134200 | BE559598 | Hs.197803 | KIAA0160 protein | 2.6 | other |
| 134206 | AF107463 | Hs.79968 | splicing factor 30, survival of motor ne | 1.3 | other |
| 134219 | NM000402 | Hs.80206 | glucose-6-phosphate dehydrogenase | 1.9 | other |
| 134234 | BE300078 | Hs.80449 | Homo sapiens, clone IMAGE:3535294, mRNA, | 10.3 | SS, |
| 134275 | AI878910 | Hs.3688 | cisplatin resistance-associated overexpr | 2.5 | other |
| 134292 | AI906291 | Hs.81234 | immunoglobulin superfamily, member 3 | 1.3 | TM |
| 134301 | AW502505 | Hs.81360 | Homo sapiens cDNA: FLJ21927 fis, done H | 1.6 | TM |
| 134305 | U61397 | Hs.81424 | ubiquitin-like 1 (sentrin) | 2.1 | TM |
| 134324 | AB029023 | Hs.179946 | KIAA1100 protein | 5.3 | ? |
| 134326 | AW903838 | Hs.81800 | chondroifin sulfate proteoglycan 2 (vers | 2.5 | TM |
| 134329 | N92036 | Hs.81848 | RAD21 (S. pombe) homolog | 3.9 | ? |
| 134337 | NM_004922 | Hs.81964 | SEC24 (S. cerevisiae) related gene famil | 2.4 | TM |
| 134348 | AW291946 | Hs.82065 | interieukin 6 signal transducer (gp130, | 6.8 | TM |
| 134367 | AA339449 | Hs,82285 | phosphoribosylglycinamide formyltransfer | 2.3 | TM |
| 134376 | X06560 | Hs.82396 | 2,5-oligoadenylate synthetase 1 (40-46 | 5.5 | other |
| 134379 | AW362124 | Hs.323193 | hypothetical protein MG03222 | 5.9 | TM |
| 134384 | AI589941 | Hs.8254 | Homo sapiens, Similar to tumor different | 2.2 | other |
| 134391 | AA417383 | Hs.82582 | integrin, beta-like 1 (with EGF-like rep | 2.1 | other |
| 134395 | AA456539 | Hs.8262 | lysosomal | 2.3 | other |
| 134405 | AW067903 | Hs.82772 | collagen, type XI, alpha 1 | 72.9 | other |
| 134411 | BE272095 | Hs.167791 | reticulocalbin 1, EF-hand calcium bindin | 4.4 | other |
| 134415 | AI750762 | Hs.82911 | protein tyrosine phosphatase type IVA, m | 2.3 | other |
| 134421 | AU077196 | Hs.82985 | collagen, type V, alpha 2 | 6.8 | ? |
| 134424 | Z44190 | Hs.83023 | peroxisomal biogenesis factor 118 | 2.4 | other |
| 134446 | M112036 | Hs.83419 | KIAA0252 protein | 2.9 | other |
| 134447 | M58603 | Hs.83428 | nuclear factor of kappa light polypeptid | 6.7 | other |
| 134470 | X54942 | Hs.83758 | CDC28 protein kinase 2 | 2.4 | other |
| 134480 | NM_005000 | Hs.83916 | Empirically selected from AFFX single pr | 6.3 | ? |
| 134485 | X82153 | Hs.83942 | cathepsin K (pycnodysostosis) | 1.9 | other |
| 134498 | AW246273 | Hs.84131 | threonyl-tRNA synthetase | 1.8 | other |
| 134513 | AA425473 | Hs.84429 | KIAAO971 protein | 1.4 | other |
| 134516 | AK001571 | Hs.273357 | hypothetical protein FLJ10709 | 1.4 | other |
| 134520 | BE091005 | Hs.74861 | activated RNA polymerase II transcriptio | 5.6 | other |
| 134529 | AW411479 | Hs.848 | FK506-binding protein 4 (59kD) | 2.8 | ? |
| 134577 | BE244323 | Hs.85951 | exportin, tRNA (nuclear expert receptor | 1.7 | other |
| 134582 | AA927177 | Hs.86041 | CGG triplet repeat binding protein 1 | 1.7 | TM |
| 134612 | AW068223 | Hs.171581 | ubiquitin C-terminal hydrolase UCH37 | 2.1 | other |
| 134624 | AF03S119 | Hs.8700 | deleted in liver cancer 1 | 1.3 | other |
| 134632 | X78520 | Hs.174139 | chloride channel 3 | 2.1 | ? |
| 134654 | AK001741 | Hs.8739 | hypothetical protein FLJ10879 | 2.3 | other |
| 134666 | BE391929 | Hs.8752 | transmembrane protein 4 | 4 | other |
| 134687 | U62317 | Hs.88251 | arylsulfatase A | 6.2 | other |
| 134692 | NNL003474 | Hs.8850a | disintegrin and metalloproteinase doma | 2 | other |
| 134705 | BE161887 | Hs.88799 | anaphase-promoting complex subunit 10 | 1.3 | SS, |
| 134714 | Y14768 | Hs.890 | lysosomal | 7.2 | ? |
| 134719 | AA852985 | Hs.89232 | chromobex homolog 5 (Drosophila HP1 alph | 3.2 | other |
| 134722 | AF129536 | Hs.284226 | F-box only protein 6 | 2.5 | other |
| 134746 | X07871 | Hs.89476002 | antigen (p50), sheep red blood cell | 5 | other |
| 134751 | AW630803 | Hs.89497 | lamin B1 | 6.1 | other |
| 134790 | BE002798 | Hs.287850 | integral membrane protein 1 | 5.6 | TM |
| 134834 | AW451370 | Hs.8991 | adaptor-related protein complex 1, gamma | 5.3 | other |
| 134850 | AI701162 | Hs.90207 | hypothetical protein MGC11138 | 9.1 | other |
| 134853 | BE268326 | Hs.90280 | 5-aminoimidazole-4-carboxamide ribonucle | 2.4 | other |
| 134880 | AI879195 | Hs.90606 | 15 kDa selenoprotein | 2.7 | other |
| 134925 | AW885909 | Hs.6975 | PRO1073 protein | 1.5 | other |
| 134955 | AW401361 | Hs.91773 | protein phosphatase 2 (formerly 2A), cat | 4.9 | other |
| 134971 | AI097346 | Hs.286049 | phosphoserine aminotransferase | 2 | other |
| 134975 | R50333 | Hs.92186 | Leman coiled-coil protein | 2.6 | TM |
| 135011 | AB037835 | Hs.92991 | KIAA1414 protein | 1.4 | ? |
| 135022 | NM000408 | Hs.93201 | glycerol-3-phosphate dehydrogenase 2 (mi | 1.6 | ? |
| 135032 | AW301984 | Hs.173685 | hypothetical protein FLJ12619 | 1.4 | other |
| 135077 | AW503733 | Hs.9414 | KIAA1488 protein | 1.8 | other |
| 135083 | AB036063 | Hs.94262 | p53-inducible ribonucleotide reductase s | 2.5 | other |
| 135095 | AF027219 | Hs.9443 | zinc finger protein 202 | 1.5 | TM |
| 135096 | AA081258 | Hs.132390 | zinc finger protein 36 (KOX 18) | 2.1 | other |
| 135153 | AI093155 | Hs.95420 | JM27 protein | 4.4 | ? |
| 135181 | BE250865 | Hs.279529 | px19-like protein | 14.9 | ? |
| 135199 | AA477514 | Hs.96247 | translin-associated factor X | 1.3 | other |
| 135207 | N26427 | Hs.9634 | ESTs, Highly similar to C10_HUMAN PUTATI | 1.7 | other |
| 135214 | 178802 | Hs.96560 | hypothetical protein FLJ11656 | 8.2 | other |
| 135243 | BE463721 | Hs.97101 | putative G protein-coupled receptor | 2.8 | TM |
| 135245 | AI028767 | Hs.262603 | ESTs | 12.2 | TM |
| 135257 | AW291023 | Hs.97255 | ESTs, Weakly similar to A46010 X-linked | 7.7 | TM |
| 135263 | AI088775 | Hs.55498 | geranylgeranyl diphosphate synthase 1 | 1.8 | other |
| 135274 | AA448460 | Hs.112017 | GE36 gene | 4.2 | SS |
| 135294 | AA150320 | Hs.9800 | protein kinase Njmu-R1 | 1.2 | other |
| 135295 | AI090838 | Hs.98006 | ESTs | 4.9 | other |
| 135307 | AI743770 | Hs.98368 | ESTs, Weakly similar to KIAA0822 protein | 5.9 | ? |
| 135321 | AI652069 | Hs.98614 | ribosome binding protein 1 (dog 180kD ho | 12.3 | TM |
| 135354 | AA456454 | Hs.183418 | cell division cycle 2-like 1 (PITSLRE pr | 5.8 | ? |
| 135361 | AA373452 | Hs.167700 | Homo sapiens cDNA FLJ10174 fis, clone HE | 8.1 | other |
| 135389 | U05237 | Hs.99872 | fetal Alzheimer antigen | 1.9 | other |
| 135400 | X78592 | Hs.99915 | androgen receptor (dihydrotestosterone r | 13.9 | TM |
| 302256 | AA857131 | Hs.171595 | HIV TAT specific factor 1 | 1.6 | other |
| 302276 | AW057736 | Hs.323910 | HER2 receptor tyrosine kinase (c-erb-b2, | 5.3 | other |
| 303135 | AW592789 | Hs.279474 | HSPC070 protein | 2.2 | TM |
| 303686 | AK000714 | Hs.109441 | MSTP033 protein | 1.4 | SS, |
| 310085 | R43191 | Hs.101248 | Homo sapiens clone IMAGE:32553, mRNA seq | 5.2 | other |
| 315518 | AA808229 | Hs.167771 | ESTs | 2.3 | ? |
| 317781 | NM_007057 | Hs.42650 | ZW10 interactor | 2.9 | ? |
| 320836 | AI268997 | Hs.197289 | rab3 GTPase-activating protein, non-cata | 2 | other |
| 321114 | AA902256 | Hs.78979 | Golgi apparatus protein 1 | 5.6 | SS |
| 322221 | N24236 | Hs.179662 | nucleosome assembly protein 1-like 1 | 1.4 | ? |
| 322474 | AF118083 | Hs.29494 | PRO1912 protein | 1.3 | other |
| 322556 | BE041451 | Hs.177507 | hypothetical protein | 2.9 | SS, |
| 323541 | AF292100 | Hs.104613 | RP42 homolog | 1.6 | other |
| 407827 | BE278431 | Hs.40323 | BUB3 (budding uninhibited by benzimidazo | 1.8 | other |
| 408196 | AL034548 | Hs.43627 | SRY (sex determining region Y)-box 22 | 1.6 | other |
| 408813 | AI580090 | Hs.48295 | RNA helicase family | 6.2 | other |
| 409176 | R73727 | Hs.101617 | ESTs, Weakly similar to T32527 hypotheti | 5.7 | other |
| 409487 | H19886 | | gb:yn57a05.r1 Soares adult brain N2b5HB5 | 2.7 | other |
| 413670 | AB000115 | Hs.75470 | hypothetical protein, expressed in osteo | 2.6 | ? |
| 414108 | AI267592 | Hs.75761 | SFRS protein kinase 1 | 2.4 | TM |
| 414846 | AW304454 | Hs.77495 | UBX domain-containing 1 | 2.4 | other |
| 416040 | AW819158 | Hs.289044 | Homo sapiens cDNA FLJ12048 fis, clone | 2.3 | other |
| 416980 | AA381133 | Hs.80684 | high-mobility group (nonhistone chromoso | 4.2 | TM |
| 417378 | R57256 | Hs.82037 | TATA box binding protein (TBP)-associate | 23.6 | other |
| 418283 | S79895 | Hs.83942 | cathepsin K (pycnodysostosis) | 5.8 | other |
| 418467 | NM006910 | Hs.85273 | retinoblastoma-binding protein 6 | 1.3 | other |
| 420269 | U72937 | Hs.96264 | alpha thalassemia/mental retardation syn | 1.6 | ? |
| 420802 | U22376 | Hs.1334 | v-myb avian myeloblastosis viral oncogen | 2.3 | ? |
| 421225 | AA463798 | Hs.102696 | MCT-1 protein | 1.6 | ? |
| 421642 | AF172066 | Hs.106346 | retinoic acid repressible protein | 3.5 | other |
| 421828 | AW891965 | Hs.279789 | histone deacetylase 3 | 5 | other |
| 421983 | AI252640 | Hs.110354 | peptidylprolyl isomerase C (cyclophilin | 3.1 | TM |
| 422052 | AA302744 | Hs.104518 | ESTs | 1.9 | TM |
| 422055 | NM_014320 | Hs.111029 | putative heme-binding protein | 2.4 | other |
| 423750 | AF165883 | Hs.298229 | prefoldin 2 | 4.2 | ? |
| 424001 | W67883 | Hs.137476 | paternally expressed 10 (PEG10; KIAA105 | 7.1 | ? |
| 425182 | AF041259 | Hs.155040 | zinc finger protein 217 | 2.3 | other |
| 425284 | AF155568 | Hs.155489 | NS1-associated protein 1 | 3.5 | other |
| 426372 | BE304680 | Hs.169531 | DEAD/H (Asp-Glu-Ala-Asp/His) box polypep | 1.9 | ? |
| 428049 | AW183765 | Hs.182238 | GW128 protein | 7.6 | ? |
| 428477 | AW500533 | Hs.11482 | splicing factor, arginine/serine-rich 11 | 1.7 | other |
| 437562 | AB001636 | Hs.5683 | DEAD/H (Asp-Glu-Ala-Asp/His) box polypep | 2.4 | other |
| 438449 | AK001333 | Hs.6216 | Homo sapiens hepatocellular carcinoma-as | 3.8 | other |
| 441560 | F13386 | Hs.7888 | Homo sapiens clone 23736 mRNA sequence | 5.6 | other |
| 445580 | AF167572 | Hs.12912 | skb1 (S. pombe) homolog | 2 | TM |
| 446999 | AA151520 | Hs.334822 | hypothetical protein MGC4485 | 7.6 | other |
| 447111 | AI017574 | Hs.17409 | cysteine-rich protein 1 (intestinal) | 2.2 | other |
| 447778 | BE620592 | Hs.71190 | ESTs, Weakly similar to S16506 hypotheti | 2.9 | other |
| 448873 | NM_003677 | Hs.22393 | density-regulated protein | 1.8 | other |
| 449687 | W68520 | Hs.331328 | intermediate filament protein syncoilin | 5.9 | other |
| 450701 | H39960 | Hs.288467 | Homo sapiens cDNA FLJ12280 fis, clone MA | 5.7 | other |
| 450703 | AA011202 | Hs.184771 | nuclear factor I/C (CCAAT-binding transc | 1.4 | other |
| 452461 | N78223 | Hs.108106 | transcription factor | 4.8 | ? |
| 452511 | BE408178 | Hs.285165 | Homo sapiens cDNA FLJ20845 fis, clone AD | 2.9 | other |
| 453157 | AF077036 | Hs.31989 | DKFZP586G1722 protein | 12.1 | SS, TM |
| 453658 | BE541906 | Hs.87819 | Homo sapiens, clone MGC:2492, mRNA, comp | 4.8 | other |
| 100685 | AA328229 | Hs.184582 | ribosomal protein 124 | 1.8 | TM |
| 100690 | AA383256 | Hs.1657 | estrogen receptor 1 | 1.6 | other |
| 100833 | AF135168 | Hs.108802 | N-ethylmaleimide-sensitive factor | 1.3 | other |
| 100850 | AA836472 | Hs.297939 | cathepsin B | 1.7 | ? |
| 101161 | NM_006262 | Hs.37044 | peripherin | 16.9 | other |
| 102481 | U50360 | | gb:Human calcium, calmodulin-dependent p | 3.2 | other |
| 102831 | M262170 | Hs.80917 | adaptor-related protein complex 3, sigma | 2 | ? |
| 103549 | BE270465 | Hs.78793 | protein kinase C, zeta | 8 | other |
| 103749 | AL135301 | Hs.8768 | hypothetical protein FLJ10849 | 1.8 | other |
| 104331 | AB040450 | Hs.279862 | cdk inhibitor p21 binding protein | 2 | ? |
| 104532 | AI498763 | Hs.203013 | hypothetical protein FLJ12748 | 2.1 | other |
| 104563 | AI117403 | Hs.306189 | DKFZP434F1735 protein | 1.2 | other |
| 105032 | AA127818 | | gb:z112a02.s1 Soares_pregnant_uterus_NbH | 7 | ? |
| 105039 | AA907305 | Hs.36475 | ESTs | 2.6 | ? |
| 106531 | AA454036 | Hs.8832 | ESTs | 1.6 | other |
| 106977 | AL043152 | Hs.50421 | KIAA0203 gene product | 4.9 | other |
| 107298 | N95657 | Hs.6820 | ESTs, Moderately similar to YOJ1_CAEEL H | 2.5 | TM |
| 108717 | AA122393 | Hs.70811 | hypothetical protein FLJ20516 | 1.3 | other |
| 110018 | AW579842 | Hs.104557 | hypothetical protein FLJ10697 | 5.3 | TM |
| 110330 | AI288666 | Hs.16621 | DKFZP434I116 protein | 6.3 | other |
| 111391 | NM_003896 | Hs.225939 | sialyltransferase 9 (CMP-NeuAc:lactosylc | 5.1 | SS, |
| 111392 | W46342 | Hs.325081 | Homo sapiens, clone IMAGE:3659680, mRNA, | 8.4 | other |
| 113554 | AW503990 | Hs.142442 | HP1-BP74 | 3.7 | TM |
| 113722 | AV653556 | Hs.184411 | albumin | 1.3 | other |
| 115008 | AK001827 | Hs.87889 | helicase-moi | 2 | other |
| 115062 | AA253314 | Hs.154103 | LIM protein (similar to rat protein kina | 1.5 | other |
| 115121 | AI634549 | Hs.88155 | ESTs | 2.8 | other |
| 117881 | AF161470 | Hs.260622 | butyrate-induced transcript 1 | 5.8 | TM |
| 119075 | M10905 | Hs.287820 | fibronectin 1 | 5.7 | other |
| 119615 | AL034423 | Hs.75875 | ubiquitin-conjugating enzyme E2 variant | 1.3 | other |
| 120253 | AA131376 | Hs.326401 | tibroblast growth factor 126 | 38.9 | other |
| 125006 | BE065136 | Hs.145696 | splicing factor (CC1.3) | 2.9 | ? |
| 127609 | X80031 | Hs.530 | collagen, type IV, alpha 3 (Goodpasture | 1.8 | other |
| 128868 | AA419008 | Hs.106730 | chromosome 22 open reading frame 3 | 3 | other |
| 128891 | F34856 | Hs.292457 | Homo sapiens, clone MGC:16362, mRNA, com | 13.3 | other |
| 128959 | AI580127 | Hs.107381 | hypothetical protein FLJ11200 | 10.9 | other |
| 129209 | R62676 | Hs.17820 | Rho-associated, coiled-coil containing p | 2.4 | other |
| 129449 | AI096988 | Hs.111554 | ADP-ribosylation factor-like 7 | 8.2 | TM |
| 129453 | AW974265 | Hs.111632 | Lsm3 protein | 3.3 | ? |
| 129629 | AK000398 | Hs.11747 | hypothetical protein FLJ20391 | 3.9 | other |
| 129917 | M30773 | Hs.278540 | protein phosphatase 3 (formerly 26), reg | 5.3 | TM |
| 129922 | AF042379 | Hs.13386 | gamma-tubulin complex protein 2 | 4.6 | other |
| 129989 | AB015856 | Hs.247433 | activating transcription factor 6 | 4 | SS, |
| 130182 | BE267033 | Hs.192853 | ubiquitin-conjugating enzyme E2G 2 (homo | 4.6 | other |
| 130365 | W56119 | Hs.155103 | eukaryotic translation initiation factor | 11 | other |
| 130471 | AL121438 | Hs.183706 | adducin I (alpha) | 2.7 | other |
| 130542 | U64675 | Hs.179825 | RAN binding protein 2-like 1 | 7.9 | other |
| 130586 | AB007891 | Hs.16349 | KIAA0431 protein | 5.6 | TM |
| 130768 | AF258627 | Hs.211562 | ATP-binding cassette, sub-family A (ABC1 | 5.2 | other |
| 130992 | BE398091 | Hs.74316 | desmoplakin (DPI, DPII) | 1.8 | TM |
| 131047 | H23230 | Hs.22481 | ESTs, Moderately similar to A46010 X-lin | 1.7 | ? |
| 131135 | NM_016569 | Hs.267182 | TBX3-iso protein | 3.3 | TM |
| 131339 | AF058696 | Hs.25812 | Nijmegen breakage syndrome 1 (nibrin) | 2.6 | other |
| 131760 | X76732 | Hs.3184 | nucleobindin 2 | 2.9 | TM |
| 131774 | 8E267158 | Hs.169474 | DKFZP586J0119 protein | 5.6 | other |
| 131853 | AI681917 | Hs.3321 | ESTs, Highly similar to IRX1_HUMAN IROQU | 1.3 | other |
| 131881 | AW361018 | Hs.3383 | upstream regulatory element binding prot | 3.2 | TM |
| 131887 | W17064 | Hs.332848 | SWI/SNF related, matrix associated, acti | 3.2 | other |
| 132031 | AF193844 | Hs.3758 | COP9 complex subunit 7a | 5.9 | ? |
| 132192 | AA206153 | Hs.4209 | mitochondrial ribosomal protein L37 | 2.2 | TM |
| 132203 | NM_004782 | Hs.194714 | synaptosomal-associated protein, 29kD | 7.9 | ? |
| 132240 | AB018324 | Hs.42676 | KIAA0781 protein | 4.3 | other |
| 132348 | AW067708 | Hs.170311 | heterogeneous nuclear ribonucleoprotein | 12.5 | other |
| 132528 | T78736 | Hs.50758 | SMC4 (structural maintenance of chromoso | 7.4 | ? |
| 132571 | AW674699 | Hs.5169 | suppressor of G2 allele of SKP1, S. cere | 6.9 | other |
| 132726 | N52298 | Hs.55608 | hypothetical protein MGC955 | 14.3 | ? |
| 132863 | BE268048 | Hs.236494 | RAB10, member RAS oncogene family | 10.3 | other |
| 133016 | AI439688 | Hs.6289 | hypothetical protein FLJ20886 | 4.4 | other |
| 133053 | AI065016 | Hs.6390 | Homo sapiens clone FLB3344 PRO0845 mRNA, | 1.8 | SS, TM |
| 133197 | AI275243 | Hs.180201 | hypothetical protein FLJ20671 | 1.8 | other |
| 133240 | AK001489 | Hs.242894 | ADP-ribosylation factor-like 1 | 1.8 | other |
| 133266 | AI160873 | Hs.69233 | zinc finger protein | 16.1 | other |
| 133285 | M76477 | Hs.289082 | GM2 ganglioside activator protein | 10.4 | SS, |
| 133383 | BE313555 | Hs.7252 | KIAA1224 protein | 1.5 | ? |
| 133540 | AL037159 | Hs.74619 | proteasome (prosome, macropain) 26S subu | 1.7 | other |
| 133784 | BE622743 | Hs.301064 | arfaptin 1 | 12.1 | other |
| 133791 | M34338 | Hs.76244 | spermidine synthase | 9.7 | other |
| 133850 | W29092 | Hs.7678 | cellular retinoic acid-binding protein 1 | 4.2 | SS, |
| 133859 | U86782 | Hs.178761 | 26S proteasome-associated padi homolog | 2.2 | other |
| 133881 | U30872 | Hs.77204 | centromere protein F (350/400kD, mitosin | 9.1 | other |
| 134208 | NM_000288 | Hs.79993 | peroxisomal biogenesis factor 7 | 3.2 | other |
| 134403 | AA334551 | Hs.82767 | sperm specific antigen 2 | 1.4 | other |
| 134724 | AF045239 | Hs.321576 | ring finger protein 22 | 1.4 | other |
| 134806 | AD001528 | Hs.89718 | spermine synthase | 2.6 | other |
| 134859 | D26488 | Hs.90315 | KIAA0007 protein | 13.3 | other |
| 135193 | X95525 | Hs.96103 | TATA box binding protein (TBP)-associate | 3.1 | other |
| | AA243007 | | ESTs | 1.6 | ? |
| | T70541 | | ESTs | 2.5 | SS, |
| | X57766 | | Human stromelysin-3 mRNA | 4.5 | other |
| | S66431 | | Homo sapiens clone 23592 mRNA sequence | 3.1 | other |
| | M453483 | | ESTs | 4.6 | TM |
| | R63925 | | ESTs | 1.4 | other |
| | M173417 | | ESTs | 1.9 | other |
| | AA280588 | | ESTs | 2.2 | other |
| | AA504223 | | ESTs Highly similar to CHROMOSOME | 2.4 | other |
| | AA609996 | | ESTs Highly similar to Surf-4 protein [M.musculus] | 5.5 | ? |
| | F02907 | | ESTs | 2.3 | TM |
| | AA480103 | | ESTs Weakly similar to !!!! ALU SUBFAMILY J | 2.8 | TM |
| | AA024664 | | Human NADH:ubiquinone oxidoreductase subunit | 6.2 | other |
| | AA251776 | | ESTs | 2.3 | other |
| | AA399047 | | ESTs | 2.4 | other |
| | N34059 | | EST - RC_N34059 | 3.3 | other |
| | U95367 | | Human GABA-A receptor pi subunit mRNA complete cds | 1.7 | TM |
| | AA490899 | | ESTs | 3.3 | other |
| | T54762 | | ESTs | 2.9 | ? |
| | Z41963 | | Homo sapiens HP protein (HP) mRNA complete cds | 1.3 | ? |
| | M521186 | | ESTs | 1.6 | TM |
| | AA400195 | | ESTs | 1.3 | other |
| | AA045083 | | VITAMIN K-DEPENDENT GAMMA-CARBOXYLASE | 2.5 | other |
| | AA099589 | | Homo sapiens mRNA for GDP dissociation inhibitor beta | 1.6 | TM |
| | W85712 | | ESTs Weakly similar to PROCOLLAGEN ALPHA 2(IV | 2.6 | TM |
| | W45728 | | ESTs Highly similar to HETEROGENEOUS | 3.7 | other |
| | U61232 | | Human tubulin-folding cofactor E mRNA complete cds | 2.1 | other |
| | AA425154 | | ESTs | 5.3 | other |
| | T39176 | | ESTs Weakly similar to ZK1058.4 [C.elegans] | 2.6 | SS, TM |
| | AA496000 | | ESTs | 1.9 | SS, |
| | W38150 | | EST-RC_W38150 | 1.7 | ? |
| | T96595 | | EST - RC_T96595 | 1.8 | TM |
| | AA227463 | | ESTs Weakly similar to No definition line found [C.elegans] | 1.9 | ? |
| | R46025 | | ESTs | 2.8 | SS, |
| | AA233177 | | ESTs | 2 | other |
| | AA338760 | | ESTs | 1.3 | ? |
| | AA412106 | | ESTs | 6.2 | other |
| | L47276 | | EST - L47276 | 3.4 | other |
| | D82307 | | ESTs Weakly similar to TH1 protein [D.melanogaster] | 11.4 | other |
| | AA293568 | | ESTs | 1.5 | other |
| | R37778 | | ESTs | 2.4 | other |
| | AA250843 | | Interferon regulatory factor 5 | 14.6 | ? |
| | W49521 | | Human prolyl 4-hydroxylase alpha (II) subunit | 6.5 | ? |
| | D80000 | | Human mRNA for KIAA0178 gene partial cds | 2 | other |
| | R99978 | | ESTs Weakly similar to line-1 protein ORF2 [H.sapiens] | 6.1 | ? |
| | AA195036 | | Human Ro/SSA ribonudeoprotein homolog (RoRet | 5.3 | ? |
| | Z38501 | | ESTs Weakly similar to PROBABLE E5 | 1.4 | other |
| | U37547 | | Human IAP homolog B (MIHB) mRNA complete cds | 3.2 | other |
| | AA479961 | | ESTs | 1.7 | other |
| | X57579 | | Inhibin beta A (activin A activin AB alpha polypeptide) | 15.8 | ? |
| | AA449071 | | ESTs | 1.3 | TM |
| | N51855 | | ESTs Moderately similar to NAD(+)ADP- | 1.3 | other |
| | AA421213 | | ESTs Weakly similar to F28F8.3 [C.elegans] | 3.2 | other |
| | AA355201 | | ESTs | 1.2 | SS, TM |
| | N78717 | | H. sapiensmRNA for translin | 1.5 | ? |
| | N73808 | | ESTs | 5 | ? |
| | U66782 | | Human 26S proteasome-associated pad1 | 2.2 | other |
| | AA234817 | | ESTs | 1.3 | other |
| | D13666 | | Homo sapiens mRNA for osteoblast specific | 7.5 | SS, |
| | AA236177 | | ESTs | 7.1 | ? |
| | U50648 | | Protein kinase interferon-inducible double | 4.1 | ? |
| | M28211 | | Homo sapiens GTP-binding protein (RAB4) | 2.9 | other |
| | AA446949 | | ESTs | 2.2 | other |
| | W03007 | | ESTs | 1.2 | other |
| | W61011 | | ESTs | 1.2 | other |
| | W87544 | | ESTs | 1.2 | other |
| | X02751 | | Neumblastoma RAS viral (v-ras) oncogene homolog | 1.2 | ? |
| | Z14077 | | YY1 transcription factor | 1.2 | other |
| | Z38839 | | ESTs | 1.2 | ? |
| | AA410894 | | ESTs | 1.7 | other |
| | AA504499 | | ESTs Highly similar to probable chloride channel 3 [H.sap | 1.3 | other |

TABLE 7A
Table 7 A shows the accession numbers for those pkeys lacking
unigeneID's for Table 7. For each probeset, we have listed the gene
cluster number from which the oligonucleotides were designed. Gene
clusters were compiled using sequences derived from Genbank ESTs
and mRNAs. These sequences were clustered based on sequence similarity
using Clustering and Alignment Tools (DoubleTwist, Oakland California).
The Genbank accession numbers for sequences comprising each cluster are
listed in the “Accession” column.

| | |
|---|---|
| Pkey: | Unique Eos probeset identifier number |
| CAT number: | Gene cluster number |
| Accession: | Genbank accession numbers |

| Pkey | CAT number | Accession |
|---|---|---|
| 102481 | 31281_-28 | U50360 |
| 105032 | genbank_AA127818 | AA127818 |
| 409487 | 1134778_1 | H19886 AW402806 T10231 |

TABLE 8
FIG. 8 from BRCA 001-1 US

| Pkey | ExAccn | UnigeneID | Unigene Title | R1 | R2 |
|---|---|---|---|---|---|
| 100075 | AF152333 | Hs.284160 | protocadherin gamma subfamily B, 4 | 1 | 3.8 |
| 100229 | AV652249 | Hs.180107 | polymerase (DNA directed), beta | 1.7 | 5.3 |
| 100262 | D38500 | Hs.278468 | postmeiotic segregation increased 2-like | 0.8 | 4.8 |
| 100271 | BE160081 | Hs.256290 | S100 calcium-binding protein A11 (calgiz | 3.2 | 2.3 |
| 100355 | AI907114 | Hs.71465 | squalene epoxidase | 3.3 | 1.4 |
| 100522 | X51501 | Hs.99949 | prolactin-induced protein | 11.9 | 0.4 |
| 100552 | AA019521 | Hs.301946 | lysosomal | 3.8 | 1.2 |
| 100599 | X77343 | Hs.334334 | transcription factor AP-2 alpha (activat | 9.4 | 9.4 |
| 100676 | X02761 | Hs.287820 | fibronectin 1 | 3 | 7.8 |
| 100690 | AA383256 | Hs.1657 | estrogen receptor 1 | 4.4 | 4.4 |
| 100895 | U01351 | Hs.75772 | nuclear receptor subfamily 3, group C, m | 1 | 3.9 |
| 101046 | K01160 | | NM_002122: Homo sapiens major histocompat | 1.7 | 4 |
| 101086 | AA382524 | Hs.250959 | histatin 1 | 0.8 | 4.1 |
| 101148 | NM_002923 | Hs.78944 | regulator of G-protein signalling 2, 24 k | 1.2 | 12 |
| 101161 | NM_006262 | Hs.37044 | peripherin | 3.1 | 1.1 |
| 101201 | L22524 | Hs.2256 | matrix metalloproteinase 7 (MMP7; uterin | 4.4 | 0.6 |
| 101212 | AI186220 | Hs.83164 | collagen, type XV, alpha 1 | 3.1 | 3.4 |
| 101441 | AW468397 | Hs.100000 | S100 calcium-binding protein A8 (calgran | 0.9 | 4.2 |
| 101447 | M21305 | | gb: Human alpha satellite and satellite 3 | 29.9 | 0.3 |
| 101469 | AA310162 | Hs.169248 | cytochrome c | 0.8 | 4.9 |
| 101567 | M33552 | Hs.56729 | lysosomal | 1 | 5.9 |
| 101600 | BE561617 | Hs.119192 | H2A histone family, member Z | 2.8 | 4 |
| 101624 | M55998 | | gb: Human alpha-1 collagen type I gene, 3 | 3.1 | 1.7 |
| 101674 | NM_002291 | Hs.82124 | laminin, beta 1 | 1.5 | 4.1 |
| 101861 | AA350659 | Hs.83347 | angio-associated, migratory cell protein | 3.1 | 1.4 |
| 101977 | AF112213 | Hs.184062 | putative Rab5-interacting protein | 1.3 | 6.9 |
| 102193 | AL036335 | Hs.313 | secreted phosphoprotein 1 (osteopontin, | 1.9 | 4.9 |
| 102199 | AA334592 | Hs.79914 | lumican | 2.2 | 3.8 |
| 102304 | AF015224 | Hs.46452 | mammaglobin 1 | 4.2 | 0.7 |
| 102345 | NM_003480 | Hs.300946 | Microfibril-associated glycoprotein-2 | 1.1 | 4.2 |
| 102457 | NM_001394 | Hs.2359 | dual specificity phosphatase 4 | 4.5 | 0.5 |
| 102534 | U96759 | Hs.198307 | von Hippel-Lindau binding protein 1 | 1.4 | 4.2 |
| 102541 | AI379954 | Hs.79025 | KIAA0096 protein | 0.9 | 3.9 |
| 102827 | BE244588 | Hs.6456 | chaperonin containing TCP1, subunit 2 (b | 1.5 | 10.9 |
| 102962 | R50032 | Hs.159263 | collagen, type VI, alpha 2 | 2.2 | 6.2 |
| 102991 | AW293542 | Hs.75309 | eukaryotic translation elongation factor | 5.6 | 5.7 |
| 103119 | X63629 | Hs.2877 | cadherin 3, type 1, P-cadherin (placenta | 3.7 | 0.5 |
| 103175 | X69089 | Hs.79227 | myomesin (M-protein) 2 (165 kD) | 1.3 | 4 |
| 103286 | D38616 | Hs.54941 | phosphorylase kinase, alpha 2 (liver) | 1.3 | 3.8 |
| 103319 | X83492 | Hs.82359 | tumor necrosis factor receptor superfami | 0.8 | 4.6 |
| 103372 | BE536700 | Hs.4888 | seryl-tRNA synthetase | 0.9 | 8 |
| 103419 | T34708 | Hs.272927 | Sec23 (S. cerevisiae) homolog A | 1.1 | 5.1 |
| 103471 | Y00815 | Hs.75216 | protein tyrosine phosphatase, receptor t | 3.7 | 1.2 |
| 103546 | Z14244 | Hs.75752 | cytochrome c oxidase subunit VIIb | 0.9 | 4.4 |
| 103658 | NM_000088 | Hs.172928 | collagen, type I, alpha 1 | 3.2 | 3 |
| 103758 | AA084874 | | gb: zn13e04.r1 Stratagene hNT neuron (937 | 0.9 | 10 |
| 103774 | H24185 | Hs.92918 | hypothetical protein | 1.9 | 15.9 |
| 103821 | AA095971 | Hs.198793 | Homo sapiens cDNA: FLJ22463 fis, clone H | 1.2 | 3.9 |
| 103869 | BE439604 | Hs.24322 | ATPase, H+ transporting, lysosomal (vacu | 1.4 | 3.9 |
| 103980 | AW130242 | Hs.293476 | hypothetical protein FKSG44 | 1.6 | 4.1 |
| 104054 | AK001913 | Hs.7100 | hypothetical protein | 1.5 | 4.3 |
| 104115 | AF183810 | Hs.26102 | opposite strand to trichorhinophalangeal | 7 | 7 |
| 104189 | AB040927 | Hs.301804 | KIAA1494 protein | 2 | 4.6 |
| 104230 | AB002347 | Hs.15303 | KIAA0349 protein | 0.7 | 4.5 |
| 104278 | AW583693 | Hs.109253 | N-terminal acetyltransferase complex ard | 3.3 | 3.3 |
| 104295 | AW365522 | Hs.103657 | hypothetical protein PRO2219 | 2.3 | 4.2 |
| 104319 | AW804296 | Hs.9950 | Sec61 gamma | 3.1 | 7 |
| 104425 | AF283775 | Hs.35380 | x 001 protein | 4 | 1.3 |
| 104432 | X51501 | Hs.99949 | prolactin-induced protein | 3.8 | 0.6 |
| 104464 | AW966728 | Hs.54642 | methionine adenosyltransferase II, beta | 0.8 | 6.7 |
| 104479 | AK001731 | Hs.106390 | Homo sapiens mRNA; cDNA DKFZp586H0924 (f | 1.7 | 4.8 |
| 104505 | W94824 | Hs.11565 | RIKEN cDNA 2010100O12 gene | 2 | 7.5 |
| 104592 | AW630488 | Hs.325820 | protease, serine, 23 | 1.9 | 7.4 |
| 104613 | AF123303 | Hs.24713 | hypothetical protein | 1.1 | 6.3 |
| 104636 | R82252 | Hs.106106 | protein kinase (cAMP-dependent, catalyti | 1.2 | 4 |
| 104782 | AW270555 | Hs.171774 | hypothetical protein | 1.4 | 3.9 |
| 104792 | AA960961 | Hs.305953 | zinc finger protein 83 (HPF1) | 1.5 | 4.2 |
| 104848 | AA305351 | Hs.274369 | uncharacterized hypothalamus protein HAR | 1.1 | 4.1 |
| 104849 | AI279065 | Hs.241507 | ribosomal protein S6 | 1.3 | 4.6 |
| 104850 | AL133035 | Hs.8728 | hypothetical protein DKFZp434G171 | 1.2 | 3.6 |
| 104852 | W70164 | Hs.20107 | ESTs | 0.8 | 4.2 |
| 104861 | AA058630 | Hs.29759 | RNA POLYMERASE I AND TRANSCRIPT | 1.7 | 5.1 |
| | | | RELEASE | | |
| 104873 | W03831 | Hs.20597 | host cell factor homolog | 0.8 | 5.4 |
| 104891 | W44626 | Hs.30627 | ESTs | 0.7 | 6.8 |
| 104920 | AW955089 | Hs.306083 | Novel human gene mapping to chomosome 22 | 1 | 3.9 |
| 104926 | BE298808 | Hs.33363 | DKFZP434N093 protein | 3.3 | 3.3 |
| 104952 | AW076098 | Hs.74316 | desmoplakin (DPI, DPII) | 1.2 | 3.7 |
| 104963 | AB029020 | Hs.173694 | KIAA1097 protein | 1.1 | 5.5 |
| 104977 | AI392640 | Hs.18272 | amino acid transporter system A1 | 3.2 | 1.4 |
| 105030 | BE613061 | Hs.337772 | Homo sapiens, Similar to RIKEN cDNA 0610 | 1.6 | 11.4 |
| 105035 | N39760 | Hs.8859 | Homo sapiens, Similar to RIKEN cDNA 5830 | 1.5 | 7.2 |
| 105068 | BE410438 | Hs.9006 | VAMP (vesicle-associated membrane protei | 1.1 | 3.5 |
| 105159 | AF146277 | Hs.265561 | CD2-associated protein | 1.2 | 10 |
| 105178 | AA313825 | Hs.21941 | AD036 protein | 3.6 | 8.3 |
| 105182 | BE407961 | Hs.18271 | golgi phosphoprotein 3 | 1.7 | 6.8 |
| 105274 | AI554929 | Hs.281866 | ATPase, H+ transporting, lysosomal (vacu | 1.1 | 3.7 |
| 105303 | BE243327 | Hs.182626 | chromosome 22 open reading frame 5 | 1.5 | 4 |
| 105413 | AI015709 | Hs.172089 | Homo sapiens mRNA; cDNA DKFZp586I2022 (f | 1.5 | 14 |
| 105426 | W20027 | Hs.23439 | ESTs | 4.3 | 2.9 |
| 105432 | W03516 | Hs.76698 | stress-associated endoplasmic reticulum | 1.5 | 5 |
| 105443 | AA252372 | Hs.12144 | KIAA1033 protein | 1.2 | 3.6 |
| 105483 | AL137257 | Hs.23458 | Homo sapiens cDNA: FLJ23015 fis, clone L | 1.7 | 15.8 |
| 105492 | AI805717 | Hs.289112 | CGI-43 protein | 2 | 4.8 |
| 105495 | AL037715 | Hs.28785 | microfibrillar-associated protein 3 | 1.3 | 3.9 |
| 105539 | AB040884 | Hs.109694 | KIAA1451 protein | 2.7 | 11.4 |
| 105594 | AB024334 | Hs.25001 | tyrosine 3-monooxygenase/tryptophan 5-mo | 1.3 | 6.1 |
| 105623 | BE504200 | Hs.30127 | hypothetical protein | 1.7 | 4.5 |
| 105807 | AA788946 | Hs.16869 | ESTs, Moderately similar to CA1C RAT COL | 3.9 | 24.6 |
| 105812 | BE614149 | Hs.20814 | CGI-27 protein | 1.8 | 3.6 |
| 105823 | AI559444 | Hs.293960 | ESTs | 1.9 | 6.6 |
| 105831 | AA329449 | Hs.247302 | twisted gastrulation | 1.5 | 4.3 |
| 105851 | AI827976 | Hs.24391 | hypothetical protein FLJ13612 | 3.8 | 1.9 |
| 105879 | BE392914 | Hs.30503 | Homo sapiens cDNA FLJ11344 fis, clone PL | 1.7 | 4 |
| 105918 | AW028485 | Hs.26136 | hypothetical protein MGC14156 | 1.7 | 7.4 |
| 105939 | AL137728 | Hs.12258 | Homo sapiens mRNA; cDNA DKFZp434B0920 (f | 1.2 | 3.8 |
| 105941 | AB033075 | Hs.10669 | development and differentiation enhancin | 1.3 | 4.6 |
| 105969 | AB030656 | Hs.17377 | coronin, actin-binding protein, 1C | 1.1 | 5.9 |
| 105990 | AI690586 | Hs.29403 | hypothetical protein FLJ22060 | 2 | 4.6 |
| 106012 | AI240665 | Hs.8895 | ESTs | 4.1 | 1.2 |
| 106060 | NM_001329 | Hs.171391 | C-terminal binding protein 2 | 2.6 | 7 |
| 106070 | T74445 | Hs.5957 | Home sapiens clone 24416 mRNA sequence | 1.4 | 10.7 |
| 106083 | H62087 | Hs.31659 | thyroid hormone receptor-associated prot | 1.5 | 3.6 |
| 106155 | AA425414 | Hs.33287 | nuclear factor I/B | 5.4 | 1.2 |
| 106255 | BE613206 | Hs.279607 | calpastatin | 1.8 | 4 |
| 106414 | BE568205 | Hs.28827 | mitogen-activated protein kinase kinase | 5.1 | 6.1 |
| 106538 | AK000274 | Hs.278635 | HDCMA 18P protein | 1.2 | 5.9 |
| 106568 | AW051564 | Hs.28285 | patched related protein translocated in | 1.8 | 5.4 |
| 106574 | BE044325 | Hs.227280 | U6 snRNA-associated Sm-like protein | 2.3 | 11.2 |
| 106613 | N88604 | Hs.30212 | thyroid receptor interacting protein 15 | 1.2 | 3.6 |
| 106617 | H09548 | Hs.5367 | ESTs, Weakly similar to I38022 hypotheti | 0.9 | 4.4 |
| 106619 | AA459480 | Hs.23956 | hypothetical protein FLJ20502 | 1.3 | 3.6 |
| 106701 | BE387614 | Hs.25797 | splicing factor 3b, subunit 4, 49 kD | 1.6 | 7.3 |
| 106721 | AA741038 | Hs.6670 | ESTs | 1.7 | 6.1 |
| 106776 | AA206079 | Hs.6693 | hypothetical protein FLJ20420 | 1 | 5.4 |
| 106866 | AA487416 | Hs.268231 | Homo sapiens cDNA: FLJ3111 fis, clone L | 1.6 | 5.4 |
| 106868 | BE185536 | Hs.301183 | molecule possessing ankyrin repeats indu | 3.3 | 1.2 |
| 106887 | BE503373 | Hs.334335 | hypothetical protein FLJ13576 | 1.4 | 6.3 |
| 106940 | T85594 | Hs.339808 | hypothetical protein FLJ10120 | 3.3 | 1.8 |
| 106968 | AF216751 | Hs.26813 | CDA14 | 3 | 3 |
| 107052 | BE391904 | Hs.12482 | glyceronephosphate O-acyltransferase | 1.7 | 7.6 |
| 107061 | BE147611 | Hs.6354 | stromal cell derived factor receptor 1 | 1.2 | 4.3 |
| 107149 | AI289507 | Hs.299883 | hypothetical protein FLJ23399 | 1.8 | 6.5 |
| 107222 | BE172058 | Hs.82689 | tumor rejection antigen (gp96) 1 | 1.2 | 6.9 |
| 107233 | BE267795 | Hs.22595 | hypothetical protein FLJ10637 | 1.4 | 3.5 |
| 107295 | AA186629 | Hs.80120 | UDP-N-acetyl-alpha-D-galactosamine: polyp | 2.6 | 4.3 |
| 107679 | AA011510 | Hs.60512 | ESTs | 1.8 | 4 |
| 107914 | AA027229 | Hs.61329 | ESTs, Weakly similar to T16370 hypotheti | 1.3 | 3.5 |
| 107965 | AF109219 | Hs.108787 | phosphatidylinositol glycan, class N | 1.6 | 3.5 |
| 108033 | AW368993 | Hs.323748 | Homo sapiens clone CDABP0086 mRNA sequen | 1.8 | 8.1 |
| 108060 | AA291440 | Hs.73149 | paired box gene 8 | 1.1 | 3.5 |
| 108081 | AA093668 | Hs.28578 | muscleblind (Drosophila)-like | 0.7 | 5.6 |
| 108137 | AI283611 | Hs.263479 | ESTs, Weakly similar to HMG1_HUMAN HIGH | 1.2 | 5.6 |
| 108186 | AW068579 | Hs.7780 | Homo sapiens mRNA; cDNA DKFZp564A072 (fr | 3.1 | 6.9 |
| 108215 | AI879238 | Hs.299315 | collapsin response mediator protein-5; C | 1.5 | 4.6 |
| 108297 | AA333660 | Hs.71331 | hypothetical protein MGC5350 | 1.5 | 4 |
| 108339 | AW151340 | Hs.51615 | ESTs, Weakly similar to ALU7_HUMAN ALU S | 6.3 | 4.7 |
| 108371 | AA074374 | Hs.67639 | ESTs | 1.3 | 3.8 |
| 108399 | AF086070 | Hs.237519 | EST | 1 | 3.6 |
| 108469 | AA079487 | | gb: zm97f08.s1 Stratagene colon HT29 (937 | 1.5 | 3.6 |
| 108470 | AA079500 | | gb: zm96h10.s1 Stratagene colon HT29 (937 | 1.1 | 4.3 |
| 108564 | M23114 | Hs.1526 | ATPase, Ca++ transporting, cardiac muscl | 2 | 4.9 |
| 108641 | AA112059 | Hs.429 | ATP synthase, H+ transporting, mitochond | 1.1 | 3.5 |
| 108668 | AA058522 | Hs.185751 | ESTs | 1.2 | 3.6 |
| 108694 | AA036725 | Hs.61847 | ESTs | 1.4 | 3.6 |
| 108824 | AK001332 | Hs.44672 | hypothetical protein FLJ10470 | 1.4 | 3.5 |
| 108863 | AA133456 | Hs.102548 | glucocorticoid receptor DNA binding fact | 1.2 | 4 |
| 108893 | BE276891 | Hs.194691 | retinoic acid induced 3 | 1.3 | 3.6 |
| 108992 | AA152312 | Hs.72047 | ESTs | 1.1 | 4.1 |
| 109072 | AI732585 | Hs.22394 | hypothetical protein FLJ10893 | 1.2 | 3.5 |
| 109097 | AA167512 | | gb: zp10f12.s1 Stratagene fetal retina 93 | 1.3 | 5 |
| 109160 | BE220601 | Hs.301997 | hypothetical protein FLJ13033 | 4 | 6.1 |
| 109244 | BE179030 | Hs.64239 | Human DNA sequence from clone RP5-1174N9 | 1.7 | 7.4 |
| 109481 | AA878923 | Hs.289069 | hypothetical protein FLJ21016 | 3.8 | 7.7 |
| 109484 | AA366263 | Hs.72531 | hypothetical protein FLJ11838 | 1.9 | 4 |
| 109795 | AA173942 | Hs.326416 | Homo sapiens mRNA; cDNA DKFZp564H1916 (f | 3.7 | 1.3 |
| 110009 | BE075297 | Hs.6614 | ESTs, Weakly similar to A43932 mucin 2 p | 4.6 | 7.4 |
| 110107 | AW151660 | Hs.31444 | ESTs | 1.2 | 3.5 |
| 110411 | AW001579 | Hs.9645 | Homo sapiens mRNA for KIAA1741 protein, | 3.7 | 3.3 |
| 110731 | NM_014899 | Hs.188006 | KIAA0878 protein | 2.8 | 3.7 |
| 110756 | N21207 | Hs.182999 | ESTs | 1.6 | 3.5 |
| 110930 | BE242691 | Hs.14947 | ESTs | 3.1 | 1.2 |
| 110935 | AI753230 | Hs.323562 | hypothetical protein DKFZp564K142 | 1.9 | 7.5 |
| 111051 | AI681293 | Hs.12186 | hypothetical protein FLJ22558 | 2 | 4 |
| 111110 | AK001566 | Hs.23618 | hypothetical protein FLJ10704 | 1.1 | 3.8 |
| 111356 | BE301871 | Hs.4867 | mannosyl (alpha-1,3-)-glycoprotein beta- | 1 | 8.2 |
| 111357 | BE314949 | Hs.87128 | hypothetical protein FLJ23309 | 3.3 | 6.1 |
| 111770 | R27975 | Hs.269401 | ESTs, Moderately similar to S65657 alpha | 1.2 | 5.4 |
| 111900 | AF131784 | Hs.25318 | Homo sapiens clone 25194 mRNA sequence | 3.2 | 0.8 |
| 111903 | NM_014906 | Hs.166351 | KIAA1072 protein | 1 | 5.4 |
| 111951 | NM_014927 | Hs.100527 | KIAA0902 protein | 1 | 3.8 |
| 112141 | AW137198 | Hs.278682 | Phosphatidylglycerophosphate Synthase | 1.4 | 3.5 |
| 112193 | R49499 | Hs.138238 | ESTs | 1.5 | 3.6 |
| 112197 | NM_003655 | Hs.5637 | ESTs | 4.6 | 2 |
| 112610 | AW500106 | Hs.23643 | serine/threonine protein kinase MASK | 3.3 | 10.5 |
| 112971 | Z42387 | Hs.83883 | transmembrane, prostate androgen induced | 3.2 | 3 |
| 112984 | T16971 | Hs.289014 | ESTs, Weakly similar to A43932 mucin 2 p | 3.7 | 10.8 |
| 113056 | AF019226 | Hs.8036 | glioblastoma overexpressed | 4.5 | 3.7 |
| 113449 | AW160683 | Hs.158006 | hypothetical protein | 1.2 | 4.4 |
| 113497 | AF143321 | Hs.15572 | hypothetical protein IMAGE 109914 | 0.9 | 3.6 |
| 113508 | AL042936 | Hs.211571 | holocytochrome c synthase (cytochrome c | 1.1 | 3.5 |
| 113531 | AK001898 | Hs.16740 | hypothetical protein FLJ11036 | 1.2 | 3.9 |
| 113604 | AI075407 | Hs.296083 | ESTs, Moderately similar to I54374 gene | 1.7 | 5.3 |
| 113674 | NM_014214 | Hs.5753 | inositol(myo)-1(or4)-monophosphatase 2 | 0.8 | 6.1 |
| 113841 | W30681 | Hs.146233 | Homo sapiens cDNA: FLJ22130 fis, clone H | 1.7 | 6.2 |
| 113857 | AW243158 | Hs.5297 | DKFZP564A2416 protein | 1.2 | 4.6 |
| 113931 | BE255499 | Hs.3496 | hypothetical protein MGC15749 | 1.5 | 4 |
| 113936 | W17056 | Hs.83623 | nuclear receptor subfamily 1, group I, m | 3.8 | 1 |
| 113987 | AA345519 | Hs.9641 | complement component 1, q subcomponent, | 1.2 | 4.7 |
| 114132 | AI342493 | Hs.24192 | Homo sapiens cDNA FLJ20767 fis, clone CO | 0.3 | 4.3 |
| 114156 | BE179882 | Hs.336920 | glutathione peroxidase 3 (plasma) | 1.1 | 4.3 |
| 114213 | N58309 | Hs.19575 | CGI-11 protein | 1.6 | 9.2 |
| 114636 | AA075488 | | gb: zm88d01.s1 Stratagene ovarian cancer | 1.6 | 3.7 |
| 114760 | AI929382 | Hs.252692 | hypothetical protein FLJ20343 | 1.4 | 4 |
| 114781 | T10446 | Hs.95388 | ESTs | 1 | 4.3 |
| 114795 | AB037858 | Hs.173484 | hypothetical protein FLJ10337 | 1.6 | 9.2 |
| 114901 | AV660012 | Hs.196437 | hypothetical protein FLJ10788 | 1.4 | 5.2 |
| 115096 | AI683069 | Hs.175319 | ESTs | 3.7 | 1 |
| 115518 | BE541042 | Hs.23240 | Homo sapiens cDNA: FLJ21848 fis, clone H | 3.2 | 4.2 |
| 115646 | N36110 | Hs.305971 | solute carrier family 2 (facilitated glu | 1.5 | 3.9 |
| 115764 | AW582256 | Hs.91011 | anterior gradient 2 (Xenepus laevis) hom | 1.3 | 5.9 |
| 115802 | AW410233 | Hs.206521 | YME1 (S. cerevisiae)-like 1 | 1.7 | 6.6 |
| 115994 | AB037836 | Hs.109315 | KIAA1415 protein | 1.5 | 9.1 |
| 116032 | BE383668 | Hs.42484 | hypothetical protein FLJ10618 | 0.9 | 4.3 |
| 116046 | BE395293 | Hs.94491 | hypothetical protein FLJ20297 | 1.6 | 5.5 |
| 116274 | AI129767 | Hs.182874 | guanine nucleotide binding protein (G pr | 3.2 | 2.4 |
| 116310 | Z24854 | Hs.42299 | ESTs | 0.8 | 4.7 |
| 116356 | AI371223 | Hs.288671 | Homo sapiens cDNA FLJ11997 fis, clone HE | 2.4 | 3.9 |
| 116429 | AF191018 | Hs.279923 | putative nucleotide binding protein, est | 5.5 | 5.5 |
| 116461 | AA313607 | Hs.58633 | Homo sapiens cDNA: FLJ22145 fis, clone H | 5 | 1.3 |
| 116470 | AI272141 | Hs.83484 | SRY (sex determining region Y)-box 4 | 8.7 | 4.5 |
| 116578 | D21262 | Hs.75337 | nucleolar and coiled-body phosphoprotein | 3.2 | 6.9 |
| 116579 | AW888411 | Hs.81915 | leukemia-associated phosphoprotein p18 ( | 3.2 | 3 |
| 116589 | AI557212 | Hs.17132 | ESTs, Moderately similar to I54374 gene | 3.1 | 8.3 |
| 116786 | H25836 | Hs.301527 | ESTs, Moderately similar to unknown [H. s | 3.2 | 4.5 |
| 117170 | N25929 | Hs.42500 | ADP-ribosylation factor-like 5 | 7 | 5.5 |
| 117215 | N20066 | Hs.133207 | PTPRF interacting protein, binding prote | 1.2 | 6.2 |
| 117280 | M18217 | Hs.172129 | Homo sapiens cDNA: FLJ21409 fis, clone C | 4.5 | 2.4 |
| 117576 | AI383467 | Hs.44597 | ESTs | 1.4 | 4.2 |
| 117667 | U59305 | Hs.44708 | Ser-Thr protein kinase related to the my | 4.3 | 0.5 |
| 117881 | AF161470 | Hs.260622 | butyrate-induced transcript 1 | 2.1 | 5.7 |
| 118336 | BE327311 | Hs.47166 | HT021 | 3.6 | 7.7 |
| 118475 | N66845 | | gb: za46c11.s1 Soares fetal liver spleen | 4.2 | 0.5 |
| 118493 | AL353944 | Hs.50115 | Homo sapiens mRNA; cDNA DKFZp761J1112 (f | 3.5 | 3.3 |
| 118505 | N67343 | | gb: yz50b07.s1 Morton Fetal Cochlea Homo | 2.1 | 3.8 |
| 119159 | AF142419 | Hs.15020 | homolog of mouse quaking QKI (KH domain | 3.7 | 1.5 |
| 119307 | BE048061 | Hs.37054 | ephrin-A3 | 3 | 1.1 |
| 119355 | BE218319 | Hs.5807 | GTPase Rab14 | 1.1 | 5.6 |
| 119462 | BE041667 | Hs.314544 | Homo sapiens cervical cancer suppressor- | 1.4 | 4.3 |
| 119771 | AI905687 | Hs.2533 | EST | 3.2 | 1 |
| 119940 | AL050097 | Hs.272531 | DKFZP586B0319 protein | 4.3 | 0.7 |
| 119943 | BE565849 | Hs.14158 | copine III | 3.5 | 1.9 |
| 120407 | AA235207 | Hs.250456 | hypothetical protein DKFZp762F2011 | 1.5 | 3.7 |
| 120493 | AW968080 | Hs.152939 | Homo sapiens clone 24630 mRNA sequence | 4 | 1.4 |
| 120677 | AF150208 | Hs.108327 | damage-specific DNA binding protein 1 (1 | 1.6 | 6.8 |
| 120867 | AA350781 | Hs.96967 | ESTs | 1.1 | 3.6 |
| 121368 | BE262956 | Hs.178292 | KIAA0180 protein | 1.5 | 4.1 |
| 121603 | AA416785 | Hs.249495 | heterogeneous nuclear ribonucleoprotein | 2.2 | 5.5 |
| 121723 | AA243499 | Hs.104800 | hypothetical protein FLJ10134 | 3.4 | 3.2 |
| 122223 | AF169797 | Hs.27413 | adaptor protein containing pH domain, PT | 3.9 | 3.9 |
| 122378 | AB032948 | Hs.21356 | hypothetical protein DKFZp762K2015 | 1.4 | 7.1 |
| 122946 | AI718702 | Hs.308026 | major histocompatibility complex, class | 1.4 | 3.7 |
| 123155 | AF121856 | Hs.284291 | sorting nexin 6 | 1.2 | 4.9 |
| 123158 | AF161426 | Hs.218329 | hypothetical protein | 2.4 | 3.6 |
| 123327 | AA421581 | Hs.178443 | ESTs | 0.9 | 5.2 |
| 123495 | W28673 | Hs.106747 | serine carboxypeptidase 1 precursor prot | 1.3 | 5.1 |
| 123526 | AA608657 | | gb: ae55d04.s1 Stratagene lung carcinoma | 2.1 | 5.2 |
| 123533 | AA608751 | | gb: ae56h07.s1 Stratagene lung carcinoma | 2.1 | 9.3 |
| 123768 | AI932318 | Hs.188762 | ESTs, Moderately similar to H2BL_HUMAN H | 1.1 | 3.6 |
| 123961 | AL050184 | Hs.21610 | DKFZP434B203 protein | 1.1 | 3.5 |
| 123999 | AF084555 | Hs.7351 | cyclic AMP phosphoprotein, 19 kD | 1.4 | 3.8 |
| 124000 | BE563957 | Hs.74861 | activated RNA polymerase II transcriptio | 1.9 | 11.2 |
| 124038 | AB037860 | Hs.173933 | nuclear factor I/A | 1.5 | 4.4 |
| 124059 | BE387335 | Hs.283713 | ESTs, Weakly similar to S64054 hypotheti | 14.8 | 11.5 |
| 124083 | AW195237 | Hs.7734 | hypothetical protein FLJ22174 | 1.2 | 6.2 |
| 124148 | BE300094 | Hs.227751 | lectin, galactoside-binding, soluble, 1 | 2.5 | 12.7 |
| 124153 | AU077333 | Hs.160483 | erythrocyte membrane protein band 7.2 (s | 1 | 4.1 |
| 124252 | BE613340 | Hs.334725 | Homo sapiens, Similar to RIKEN cDNA 9430 | 1.5 | 8.4 |
| 124314 | AK001552 | Hs.215766 | GTP-binding protein | 1.8 | 10.2 |
| 124375 | D87454 | Hs.192966 | KIAA0265 protein | 1.1 | 4.8 |
| 124432 | N39016 | Hs.268869 | ESTs, Weakly similar to ALUC_HUMAN !!!! | 1.3 | 4.1 |
| 124447 | N48000 | | gb: yy98e12.s1 Soares_multiple_sclerosis_{—} | 2.7 | 4.3 |
| 124539 | D54120 | Hs.146409 | cell division cycle 42 (GTP-binding prot | 2.1 | 5.7 |
| 124543 | AI393320 | Hs.104573 | ESTs | 1 | 4.1 |
| 124564 | H66409 | Hs.108275 | ESTs | 1.4 | 4 |
| 124574 | AL036596 | Hs.42322 | A kinase (PRKA) anchor protein 2 | 0.7 | 4 |
| 124605 | AA749315 | Hs.77171 | minichromosome maintenance deficient (S. | 1.1 | 3.5 |
| 124639 | H60193 | Hs.21143 | DKFZP586C1324 protein | 1.4 | 3.6 |
| 124659 | AI680737 | Hs.289068 | Homo sapiens cDNA FLJ11918 fis, clone HE | 1.5 | 9.9 |
| 124737 | BE270465 | Hs.78793 | protein kinase C, zeta | 0.7 | 4 |
| 124760 | AW408586 | Hs.91052 | ESTs, Moderately similar to ALU5_HUMAN A | 0.9 | 3.6 |
| 124763 | BE410405 | Hs.76288 | calpain 2, (m/II) large subunit | 1.3 | 3.9 |
| 124792 | R44357 | Hs.48712 | hypothetical protein FLJ20736 | 1.8 | 4.2 |
| 124842 | R56485 | | gb: yg93h09.s1 Soares infant brain 1NIB H | 1 | 3.6 |
| 124940 | AF068846 | Hs.103804 | heterogeneous nuclear ribonucleoprotein | 3.2 | 3.4 |
| 124949 | AI903210 | Hs.336780 | tubulin, beta polypeptide | 1 | 4.4 |
| 124960 | AL023513 | Hs.194766 | seizure related gene 6 (mouse)-like | 0.9 | 5.2 |
| 124995 | T52700 | Hs.110044 | ESTs | 0.9 | 3.5 |
| 125030 | AA610577 | Hs.187775 | ESTs | 1.2 | 5 |
| 125034 | BE548446 | Hs.5167 | Homo sapiens mRNA; cDNA DKFZp434F152 (fr | 1.5 | 3.7 |
| 125058 | T83731 | Hs.3343 | phosphoglycerate dehydrogenase | 0.9 | 6 |
| 125076 | AA973971 | | gb: oq02h08.s1 NCI_CGAP_Lu5 Homo sapiens | 1 | 3.7 |
| 125090 | T91518 | | gb: ye20f05.s1 Stratagene lung (937210) H | 3.2 | 2.5 |
| 125103 | AA570056 | Hs.122730 | ESTs, Moderately similar to KIAA1215 pro | 5.3 | 6.6 |
| 125154 | W38419 | | gb: zc78a07.s1 Pancreatic Islet Homo sapi | 0.9 | 6.1 |
| 125155 | AA837043 | Hs.143669 | ESTs | 1.1 | 4.3 |
| 125159 | AK000669 | Hs.274428 | TRF2-interacting telomeric RAP1 protein | 1.1 | 4.1 |
| 125170 | AL020996 | Hs.8518 | selenoprotein N | 1.1 | 3.8 |
| 125181 | R40815 | Hs.12396 | ESTs, Weakly similar to 2004399A chromos | 1 | 3.6 |
| 125193 | W67577 | Hs.84298 | CD74 antigen (invariant polypeptide of m | 1.2 | 7.8 |
| 125260 | H05635 | Hs.294030 | topoisomerase-related function protein 4 | 1 | 4.9 |
| 125262 | AW884980 | Hs.171957 | triple functional domain (PTPRF interact | 1.3 | 4.8 |
| 125272 | BE612888 | Hs.180224 | myosin regulatory light chain | 1.1 | 16.1 |
| 125388 | W27235 | Hs.64311 | a disintegrin and metalloproteinase doma | 1.4 | 5.3 |
| 125824 | Z45258 | Hs.286013 | short coiled-coil protein | 2.4 | 8.7 |
| 125852 | AW630088 | Hs.76550 | Homo sapiens mRNA; cDNA DKFZp564B1264 (f | 1.8 | 4.6 |
| 125970 | AW504721 | Hs.177516 | high density lipoprotein binding protein | 1.9 | 3.8 |
| 126192 | AW160399 | Hs.30376 | hypothetical protein | 1.4 | 4.1 |
| 126469 | BE384361 | Hs.182885 | ESTs, Weakly similar to JC5024 UDP-galac | 2 | 3.7 |
| 126510 | AA057593 | Hs.334762 | hypothetical protein FLJ14735 | 1.3 | 4.1 |
| 127095 | AA340277 | Hs.10248 | Homo sapiens cDNA FLJ20167 fis, clone CO | 1.3 | 5 |
| 127524 | AI243596 | Hs.94830 | ESTs, Moderately similar to T03094 A-kin | 4.3 | 0.9 |
| 128312 | J04182 | Hs.150101 | lysosomal | 1.5 | 4.7 |
| 128453 | X02761 | Hs.287820 | fibronectin 1 | 1.2 | 4.3 |
| 128460 | T16206 | Hs.237164 | ESTs, Highly similar to LDHH_HUMAN L-LAC | 3.1 | 44.4 |
| 128491 | H08379 | Hs.165563 | hypothetical protein DKFZp434N1429 | 0.6 | 13.1 |
| 128495 | NM_005904 | Hs.100602 | MAD (mothers against decapentaplegic, Dr | 1.3 | 4 |
| 128546 | NM_003478 | Hs.101299 | cullin 5 | 1 | 5.1 |
| 128574 | AI185977 | Hs.38260 | ubiquitin specific protease 18 | 0.8 | 4 |
| 128611 | NM_014721 | Hs.102471 | KIAA0680 gene product | 1.3 | 3.7 |
| 128652 | AA432202 | Hs.103147 | hypothetical protein FLJ21347 | 1.4 | 3.9 |
| 128653 | D87432 | Hs.10315 | solute carrier family 7 (cationic amino | 1.2 | 3.6 |
| 128655 | AI246669 | Hs.324275 | WW domain-containing protein 1 | 0.8 | 4.1 |
| 128684 | BE246444 | Hs.283685 | hypothetical protein FLJ20396 | 3 | 1.6 |
| 128717 | AK001564 | Hs.104222 | hypothetical protein FLJ10702 | 2.8 | 4.8 |
| 128774 | AA476220 | Hs.54457 | CD81 antigen (target of antiproliferativ | 1.1 | 10.6 |
| 128790 | AF026692 | Hs.105700 | secreted frizzled-related protein 4 | 1 | 3.8 |
| 128805 | AA194554 | Hs.183434 | ATPase, H+ transporting, lysosomal (vacu | 5.3 | 5.3 |
| 128827 | AI638184 | Hs.106334 | Homo sapiens clone 23836 mRNA sequence | 2.2 | 5.3 |
| 128840 | AI917602 | Hs.106440 | ESTs | 1 | 4.5 |
| 128869 | AA768242 | Hs.80618 | hypothetical protein | 0.8 | 3.6 |
| 128889 | D60985 | Hs.106909 | DKFZP566D193 protein | 4.6 | 3.7 |
| 128890 | AI222020 | Hs.182364 | CocoaCrisp | 3 | 1.5 |
| 128915 | AK000140 | Hs.107139 | hypothetical protein | 0.2 | 3.9 |
| 128920 | AA622037 | Hs.166468 | programmed cell death 5 | 2.5 | 15.2 |
| 128926 | AF155096 | Hs.107213 | hypothetical protein FLJ20585 | 4 | 4 |
| 128930 | AA298958 | Hs.10724 | MDS023 protein | 1.2 | 4.5 |
| 128942 | AW247536 | Hs.10729 | hypothetical protein | 1.4 | 5 |
| 128948 | AW953622 | Hs.223025 | RAB31, member RAS oncogene family | 2.3 | 5.6 |
| 128953 | AB020716 | Hs.107362 | KIAA0909 protein | 0.9 | 3.9 |
| 128979 | AW271217 | Hs.281434 | Homo sapiens cDNA FLJ14028 fis, clone HE | 1.5 | 3.6 |
| 128980 | AA258924 | Hs.10758 | NM_002495*: Homo sapiens NADH dehydrogena | 0.8 | 3.8 |
| 129005 | AI770025 | Hs.13323 | hypothetical protein FLJ22059 | 1.2 | 5.7 |
| 129009 | C15105 | Hs.330716 | Homo sapiens cDNA FLJ14368 fis, clone HE | 2.1 | 9.9 |
| 129013 | AA371156 | Hs.107942 | DKFZP564M112 protein | 2.4 | 3.8 |
| 129068 | AI634522 | Hs.152925 | KIAA1268 protein | 1.2 | 3.8 |
| 129106 | AW504486 | Hs.108689 | sterol regulatory element binding transc | 1.2 | 5.5 |
| 129113 | BE543205 | Hs.288771 | DKFZP586A0522 protein | 0.5 | 3.7 |
| 129125 | AB002450 | Hs.278391 | CGI-109 protein | 1 | 5.2 |
| 129126 | AW881089 | Hs.108806 | Homo sapiens mRNA; cDNA DKFZp566M0947 (f | 1.5 | 7 |
| 129151 | N23018 | Hs.171391 | C-terminal binding protein 2 | 2.1 | 9.7 |
| 129230 | AA335362 | Hs.109646 | Empirically selected from AFFX single pr | 0.9 | 8.6 |
| 129234 | M18916 | Hs.282997 | glucosidase, beta; acid (includes glucos | 1.1 | 3.5 |
| 129238 | BE542214 | Hs.109697 | ESTs | 1.1 | 12.8 |
| 129239 | W57656 | Hs.109701 | ubiquitin-like 5 | 3.2 | 5.1 |
| 129241 | AI878857 | Hs.109706 | hematological and neurological expressed | 1.9 | 5.7 |
| 129243 | BE169531 | Hs.109727 | TAK1-binding protein 2; KIAA0733 protein | 1.2 | 6.6 |
| 129247 | R49920 | Hs.109733 | CGI-131 protein | 1.5 | 3.5 |
| 129250 | AA344367 | Hs.109760 | Empirically selected from multiple AFFX | 1 | 5.4 |
| 129258 | AA250970 | Hs.251946 | poly(A)-binding protein, cytoplasmic 1-l | 1.3 | 4.1 |
| 129260 | AF077200 | Hs.279813 | hypothetical protein | 1.6 | 3.9 |
| 129270 | AA357185 | Hs.109918 | ras homoleg gene family, member H | 1.8 | 4.2 |
| 129277 | AB007896 | Hs.110 | putative L-type neutral amino acid trans | 1.1 | 6.1 |
| 129284 | AA318224 | Hs.296141 | ESTs | 2.5 | 4.8 |
| 129300 | W94197 | Hs.110165 | ribosomal protein L26 homolog | 1.6 | 5.1 |
| 129318 | AF189062 | Hs.285976 | tumor metastasis-suppressor | 1.8 | 6.5 |
| 129352 | AW511656 | Hs.170177 | Meis1 (mouse) homolog | 0.9 | 4 |
| 129362 | U30246 | Hs.110736 | solute carrier family 12 (sodium/potassi | 1.4 | 9.2 |
| 129379 | BE278964 | Hs.11085 | CGI-111 protein | 1 | 4.8 |
| 129390 | AA318271 | Hs.250905 | hypothetical protein | 1 | 4.1 |
| 129416 | AA016188 | Hs.111244 | hypothetical protein | 1.8 | 10.7 |
| 129427 | AI498631 | Hs.111334 | ferritin, light polypeptide | 1.1 | 4.8 |
| 129470 | W92931 | Hs.250899 | heat shock factor binding protein 1 | 1.8 | 9.3 |
| 129472 | AL050260 | Hs.323817 | DKFZP547E1010 protein | 1 | 5 |
| 129475 | NM_004477 | Hs.203772 | FSHD region gene 1 | 1.1 | 4.2 |
| 129498 | AA449789 | Hs.75511 | connective tissue growth factor | 1.9 | 6.8 |
| 129501 | AI631811 | Hs.180403 | STRIN protein | 1.1 | 9.7 |
| 129527 | AA769221 | Hs.270847 | delta-tubulin | 1.1 | 4.3 |
| 129545 | R18087 | Hs.323769 | cisplatin resistance related protein CRR | 1 | 4.2 |
| 129579 | AW517695 | Hs.286218 | junctional adhesion molecule 1 | 2.3 | 3.5 |
| 129606 | AW968941 | Hs.166254 | hypothetical protein DKFZp566I133 | 2.4 | 4.4 |
| 129619 | AA209534 | Hs.284243 | tetraspan NET-6 protein | 3.2 | 13 |
| 129620 | D79338 | Hs.239720 | CCR4-NOT transcription complex, subunit | 1.6 | 4.6 |
| 129621 | AL110212 | Hs.301005 | purine-rich element binding protein B | 1.1 | 5.7 |
| 129634 | AB020335 | Hs.181300 | sel-1 (suppressor of lin-12, C. elegans )- | 0.9 | 4.3 |
| 129663 | AI207406 | Hs.11866 | translocase of inner mitochondrial membr | 1.9 | 4.8 |
| 129679 | AW889132 | Hs.11916 | ribokinase | 0.9 | 4.1 |
| 129688 | U53209 | Hs.24937 | transformer-2 alpha (htra-2 alpha) | 1.3 | 4.7 |
| 129691 | M26939 | Hs.119571 | collagen, type III, alpha 1 (Ehlers-Danl | 4.7 | 3.7 |
| 129712 | U46386 | Hs.12102 | sorting nexin 3 | 1.2 | 3.6 |
| 129747 | AL050272 | Hs.12305 | DKFZP566B183 protein | 1 | 8.9 |
| 129788 | BE397454 | Hs.124969 | Homo sapiens clone 24707 mRNA sequence | 1.4 | 3.6 |
| 129796 | BE218319 | Hs.5807 | GTPase Rab14 | 2.9 | 5.1 |
| 129797 | M62839 | Hs.1252 | apolipoprotein H (beta-2-glycoprotein I) | 0.3 | 5.1 |
| 129800 | AF052112 | Hs.12540 | lysosomal | 1.6 | 8.8 |
| 129834 | AL080084 | Hs.296155 | CGI-100 protein | 0.9 | 5.3 |
| 129836 | AW410233 | Hs.206521 | YME1 (S. cerevisiae)-like 1 | 1.8 | 9.9 |
| 129843 | NM_014840 | Hs.200598 | KIAA0537 gene product | 0.9 | 3.6 |
| 129874 | AA626937 | Hs.181551 | hypothetical protein MGC2594 | 1.4 | 9.5 |
| 129878 | Z43161 | Hs.283714 | 30 kDa protein | 1.1 | 6.3 |
| 129904 | AL119499 | Hs.13285 | neuronal potassium channel alpha subunit | 1 | 3.5 |
| 129917 | M30773 | Hs.278540 | protein phosphatase 3 (formerly 2B), reg | 2 | 5.1 |
| 129976 | X14008 | Hs.234734 | lysosomal | 0.9 | 4.9 |
| 129982 | Z14221 | | gb: H. sapiens germline transcript of Ig h | 1.2 | 3.6 |
| 130007 | R15917 | Hs.142570 | Homo sapiens clone 24629 mRNA sequence | 4.3 | 1.3 |
| 130060 | BE277024 | Hs.146381 | RNA binding motif protein, X chromosome | 1.6 | 3.8 |
| 130064 | X57815.comp | | Empirically selected from AFFX single pr | 1.2 | 8.2 |
| 130068 | M93143 | Hs.262869 | plasminogen-like | 1.4 | 7.9 |
| 130090 | H97878 | Hs.132390 | zinc finger protein 36 (KOX 18) | 1.4 | 12.3 |
| 130095 | AK001635 | Hs.14838 | hypothetical protein FLJ10773 | 0.2 | 4.6 |
| 130102 | W61005 | Hs.14896 | DHHC1 protein | 1 | 4.1 |
| 130112 | AA916785 | Hs.180610 | splicing factor proline/glutamine rich ( | 1.2 | 5.3 |
| 130115 | T47294 | Hs.149923 | X-box binding protein 1 | 3.8 | 0.8 |
| 130123 | NM_005095 | Hs.150390 | zinc finger protein 262 | 1 | 4.2 |
| 130150 | BE094848 | Hs.15113 | homogentisate 1,2-dioxygenase (homogenti | 0.5 | 4 |
| 130161 | R42678 | Hs.151385 | KIAA0564 protein | 1 | 3.7 |
| 130210 | M23115 | Hs.1526 | ATPase, Ca++ transporting, cardiac muscl | 0.4 | 4.4 |
| 130213 | BE278370 | Hs.15265 | heterogeneous nuclear ribonucleoprotein | 1.7 | 7.5 |
| 130215 | BE301883 | Hs.152707 | glioblastoma amplified sequence | 1 | 5.6 |
| 130232 | U29463 | | gb: Human cytochrome b561 gen | 1.2 | 4.2 |
| 130252 | U92014 | Hs.153527 | Homo sapiens pTM5 mariner-like transposo | 1.3 | 3.6 |
| 130281 | W78907 | Hs.15395 | similar to arginyl-tRNA synthetase (argi | 1.5 | 4.4 |
| 130343 | AB040914 | Hs.278628 | KIAA1481 protein | 2.9 | 7.5 |
| 130385 | AW067800 | Hs.155223 | stanniocalcin 2 | 3.2 | 0.2 |
| 130414 | AW842182 | Hs.241392 | small inducible cytokine A5 (RANTES) | 1.4 | 10.6 |
| 130417 | AW163518 | Hs.155485 | huntingtin interacting protein 2 | 1.7 | 11.7 |
| 130440 | AA852868 | Hs.132853 | KIAA0171 gene product | 1.1 | 5 |
| 130442 | NM_006245 | Hs.118244 | protein phosphatase 2, regulatory subuni | 1.4 | 4.3 |
| 130465 | AW362955 | Hs.15641 | Homo sapiens cDNA FLJ14415 fis, clone HE | 1.6 | 7.6 |
| 130479 | R44163 | Hs.12457 | hypothetical protein FLJ10814 | 0.9 | 4.1 |
| 130499 | AB007915 | Hs.158286 | KIAA0446 gene product | 1 | 3.8 |
| 130546 | AI598022 | Hs.193989 | TAR DNA binding protein | 1.3 | 4.7 |
| 130568 | AA232119 | Hs.16085 | putative G-protein coupled receptor | 1.2 | 9.4 |
| 130606 | AI652143 | Hs.288382 | hypothetical protein FLJ13111 | 1 | 4.1 |
| 130612 | BE242873 | Hs.16677 | WD repeat domain 15 | 1.1 | 3.6 |
| 130616 | AL049963 | Hs.284205 | up-regulated by BCG-CWS | 0.6 | 3.8 |
| 130623 | AL045128 | Hs.1691 | glucan (1,4-alpha-), branching enzyme 1 | 0.9 | 6.6 |
| 130629 | AL042896 | Hs.1697 | ATPase, H+ transporting, lysosomal (vacu | 0.9 | 3.9 |
| 130632 | AW073971 | Hs.238954 | ESTs, Weakly similar to KIAA1204 protein | 0.9 | 6.9 |
| 130639 | AI557212 | Hs.17132 | ESTs, Moderately similar to I54374 gene | 2.6 | 3.9 |
| 130641 | AF158555 | Hs.239189 | glutaminase | 1.2 | 13.8 |
| 130653 | AI861791 | Hs.278479 | TSPY-like | 1.3 | 4 |
| 130655 | AI831962 | Hs.17409 | cysteine-rich protein 1 (intestinal) | 2.5 | 4 |
| 130666 | AL117508 | Hs.194035 | KIAA0737 gene product | 1.3 | 6.2 |
| 130669 | AI928985 | Hs.17680 | hypothetical protein MGC1314 similar to | 1.4 | 3.9 |
| 130693 | R68537 | Hs.17962 | ESTs | 3.2 | 0.8 |
| 130694 | NM_014827 | Hs.17969 | KIAA0663 gene product | 1.1 | 4.8 |
| 130696 | AA325308 | Hs.18016 | Homo sapiens mRNA; cDNA DKFZp586H0324 (f | 1.8 | 5 |
| 130701 | Z98883 | Hs.18079 | phosphatidylinositol glycan, class Q | 1.1 | 6.7 |
| 130707 | AW190925 | Hs.203559 | hypothetical protein FLJ12701 | 1.2 | 4.1 |
| 130731 | AI932971 | Hs.18593 | Homo sapiens cDNA: FLJ21449 fis, clone C | 1.4 | 6.9 |
| 130787 | AF072813 | Hs.252831 | reticulon 3 | 1.2 | 11.2 |
| 130796 | AA088809 | Hs.19525 | hypothetical protein FLJ22794 | 1.8 | 6.8 |
| 130808 | NM_001761 | Hs.1973 | cyclin F | 1.3 | 4.1 |
| 130863 | Y10805 | Hs.20521 | HMT1 (hnRNP methyltransferase, S. cerevi | 3.2 | 5.9 |
| 130902 | AB037750 | Hs.21061 | KIAA1329 protein | 1 | 3.8 |
| 130908 | AW195747 | Hs.21122 | hypothetical protein FLJ11830 similar to | 1.3 | 7.9 |
| 130911 | BE409769 | Hs.21189 | DnaJ (Hsp40) homolog, subfamily A, membe | 2.7 | 3.7 |
| 130913 | BE390905 | Hs.21198 | translocase of outer mitochondrial membr | 1.9 | 4 |
| 130923 | H96115 | Hs.21293 | UDP-N-acteylglucosamine pyrophosphorylas | 1.9 | 10.3 |
| 130959 | AB023182 | Hs.184523 | KIAA0965 protein | 1.5 | 6.8 |
| 130967 | AA393071 | Hs.182579 | leucine aminopeptidase | 1.4 | 5.5 |
| 130975 | AA099923 | Hs.283728 | PEST-containing nuclear protein | 1.3 | 3.8 |
| 131037 | BE243101 | Hs.22391 | chromosome 20open reading frame 3 | 1.9 | 4.1 |
| 131039 | D87436 | Hs.166318 | lipin 2 | 1.6 | 3.5 |
| 131060 | AA194422 | Hs.22564 | myosin VI | 4.5 | 5 |
| 131097 | AL137682 | Hs.22937 | I-kappa-B-interacting Ras-like protein 2 | 2 | 3.7 |
| 131101 | BE387561 | Hs.22981 | DKFZP586M1523 protein | 1.6 | 4.5 |
| 131104 | W27770 | Hs.301756 | ESTs, Weakly similar to T31475 hypotheti | 0.9 | 3.5 |
| 131107 | BE620886 | Hs.75354 | GCN1 (general control of amino-acid synt | 2.1 | 4.5 |
| 131109 | BE564123 | Hs.23060 | DKFZP564F0522 protein | 1.1 | 4.6 |
| 131136 | AB033099 | Hs.23413 | KIAA1273 protein | 1.2 | 4.2 |
| 131148 | AW953575 | Hs.303125 | p53-induced protein PIGPC1 | 4.5 | 13.5 |
| 131150 | X77753 | Hs.23582 | tumor-associated calcium signal transduc | 3.4 | 0.4 |
| 131156 | AI472209 | Hs.323117 | ESTs | 0.8 | 4.9 |
| 131164 | AW013807 | Hs.182265 | keratin 19 | 3.3 | 2.4 |
| 131181 | H25094 | Hs.293663 | ESTs, Moderately similar to I38022 hypot | 0.6 | 4 |
| 131194 | AW864222 | Hs.24083 | KIAA0997 protein | 1.4 | 3.8 |
| 131199 | AW979155 | Hs.298275 | amino acid transporter 2 | 1.2 | 8.5 |
| 131215 | AL050107 | Hs.24341 | transcriptional co-activator with PDZ-bi | 0.7 | 4.7 |
| 131216 | AI815486 | Hs.243901 | Homo sapiens cDNA FLJ20738 fis, clone HE | 2.1 | 8.2 |
| 131233 | D89053 | Hs.268012 | fatty-acid-Coenzyme A ligase, long-chain | 1.7 | 3.5 |
| 131237 | AW956868 | Hs.24608 | DKFZP564D177 protein | 1.3 | 5.4 |
| 131262 | AU077158 | Hs.24930 | tubulin-specific chaperone a | 1.6 | 4.8 |
| 131263 | AU077002 | Hs.24950 | regulator of G-protein signalling 5 | 1.4 | 4.4 |
| 131367 | AI750575 | Hs.173933 | nuclear factor I/A | 3.3 | 2.2 |
| 131372 | AW293399 | Hs.144904 | nuclear receptor co-repressor 1 | 1.6 | 3.9 |
| 131373 | NM_006052 | Hs.26146 | Down syndrome critical region gene 3 | 1 | 11.1 |
| 131388 | NM_014810 | Hs.92200 | KIAA0480 gene product | 5 | 2 |
| 131492 | AI452601 | Hs.288869 | nuclear receptor subfamily 2, group F, m | 0.9 | 3.5 |
| 131493 | AW960146 | Hs.284137 | hypothetical protein FLJ12888 | 1 | 3.5 |
| 131514 | BE270734 | Hs.2795 | lactate dehydrogenase A | 2 | 6.5 |
| 131524 | AB040927 | Hs.301804 | KIAA1494 protein | 1.5 | 10.7 |
| 131528 | AU076408 | Hs.28309 | UDP-glucose dehydrogenase | 1.3 | 4.7 |
| 131534 | AF157326 | Hs.184786 | TBP-interacting protein | 1.3 | 4.9 |
| 131555 | T47364 | Hs.278613 | interferon, alpha-inducible protein 27 | 1.5 | 8 |
| 131578 | AA936296 | Hs.234265 | DKFZP586G011 protein | 1.8 | 3.5 |
| 131589 | C18825 | Hs.29191 | epithelial membrane protein 2 | 1.3 | 8.2 |
| 131609 | D83032 | Hs.169984 | nuclear protein | 2.8 | 3.9 |
| 131626 | BE514605 | Hs.289092 | Homo sapiens cDNA: FLJ22380 fis, clone H | 1.3 | 11.2 |
| 131670 | H03514 | Hs.10130 | ESTs | 1.3 | 4.8 |
| 131697 | C19034 | Hs.288613 | Homo sapiens cDNA FLJ14175 fis, clone NT | 3.2 | 9.7 |
| 131701 | AF103798 | Hs.30819 | hypothetical protein | 1.3 | 5.2 |
| 131703 | AW160865 | Hs.30888 | cytochrome c oxidase subunit VIIa polype | 1.3 | 7.8 |
| 131739 | AF017986 | Hs.31386 | secreted frizzled-related protein 2 | 10.6 | 14.7 |
| 131764 | AI805664 | Hs.31731 | peroxiredoxin 5 | 1.1 | 3.6 |
| 131781 | AF077036 | Hs.31989 | DKFZP586G1722 protein | 1.6 | 3.7 |
| 131791 | X62111 | | gb: H. sapiens VII-5 gene for immunoglobul | 1.1 | 3.5 |
| 131853 | AI681917 | Hs.3321 | ESTs, Highly similar to IRX1_HUMAN IROQU | 5.3 | 1.2 |
| 131870 | NM_014874 | Hs.3363 | KIAA0214 gene product | 0.6 | 4.2 |
| 131903 | NM_004642 | Hs.3436 | deleted in oral cancer (mouse, homolog) | 2.4 | 4.9 |
| 131913 | AW207440 | Hs.185973 | degenerative spermatocyte (homolog Droso | 2.4 | 6 |
| 131930 | AA772603 | Hs.69476 | Homo sapiens cDNA FLJ12758 fis, clone NT | 1.7 | 9.2 |
| 131941 | BE252983 | Hs.35086 | ubiquitin specific protease 1 | 0.5 | 5.2 |
| 131947 | AI123939 | Hs.182997 | ESTs | 0.7 | 4.1 |
| 131961 | AA129782 | Hs.3576 | Homo sapiens mRNA full length insert cDN | 0.9 | 4.8 |
| 131964 | AW381148 | Hs.198365 | 2,3-bisphosphoglycerate mutase | 1.1 | 6.1 |
| 131974 | AF208856 | Hs.268122 | hypothetical protein | 1.3 | 3.9 |
| 131983 | AF119665 | Hs.184011 | pyrophosphatase (inorganic) | 3.3 | 6.9 |
| 131997 | AF229181 | Hs.136644 | CS box-containing WD protein | 0.9 | 5.2 |
| 132006 | AW162336 | Hs.3709 | low molecular mass ubiquinone-binding pr | 1.2 | 3.6 |
| 132063 | BE277910 | Hs.3833 | 3′-phosphoadenosine 5′-phosphosulfate sy | 3.2 | 1.8 |
| 132065 | BE379335 | Hs.211594 | proteasome (prosome, macropain) 26S subu | 1.2 | 3.6 |
| 132071 | AF217798 | Hs.3850 | LIS1-interacting protein NUDEL; endoolig | 0.7 | 5.2 |
| 132079 | AI701457 | Hs.38694 | ESTs | 2 | 5.3 |
| 132094 | NM_016045 | Hs.3945 | CGI-107 protein | 1.2 | 4.3 |
| 132116 | AW960474 | Hs.40289 | ESTs | 3.1 | 3.1 |
| 132164 | AI752235 | Hs.41270 | procollagen-lysine, 2-oxoglutarate 5-dio | 1.8 | 3.7 |
| 132181 | AW961231 | Hs.16773 | Homo sapiens clone TCCCIA00427 mRNA sequ | 1.2 | 5 |
| 132208 | AL031709 | Hs.241575 | hypothetical protein CAB56184 | 1.4 | 4.2 |
| 132258 | AA306325 | Hs.4311 | SUMO-1 activating enzyme subunit 2 | 2 | 10.3 |
| 132303 | BE177330 | Hs.325093 | Homo sapiens cDNA: FLJ21210 fis, clone C | 1.2 | 4.1 |
| 132316 | U28831 | Hs.44566 | KIAA1641 protein | 5.9 | 1.6 |
| 132358 | NM_003542 | Hs.46423 | H4 histone family, member G | 5.8 | 1.5 |
| 132384 | AA312135 | Hs.46967 | HSPCO34 protein | 2.1 | 9.3 |
| 132397 | AA021160 | Hs.4750 | hypothetical protein DKFZp564K0822 | 1.3 | 4.6 |
| 132413 | AW361383 | Hs.260116 | metalloprotease 1 (pitrilysin family) | 2 | 4.9 |
| 132442 | AW970859 | Hs.313503 | ESTs | 1.2 | 5 |
| 132534 | BE388673 | Hs.5086 | hypothetical protein MGC10433 | 2 | 3.9 |
| 132540 | BE396290 | Hs.5097 | synaptogyrin 2 | 1.4 | 5.1 |
| 132554 | AF065391 | Hs.194718 | zinc finger protein 265 | 1.2 | 4 |
| 132575 | AV660538 | Hs.284162 | 60S ribosomal protein L30 isolog | 3 | 1.7 |
| 132585 | AF029750 | Hs.179600 | TAP binding protein (tapasin) | 1.8 | 4.7 |
| 132602 | AW606927 | Hs.5306 | hypothetical protein DKFZp586F1122 simil | 1.6 | 4.9 |
| 132608 | AA353044 | Hs.5321 | ARP3 (actin-related protein 3, yeast) ho | 1.8 | 8.1 |
| 132718 | NM_004600 | Hs.554 | Sjogren syndrome antigen A2 (60 kD, ribon | 4.2 | 2 |
| 132719 | AI264357 | Hs.55405 | hypothetical protein MGC16212 | 1.1 | 5.3 |
| 132730 | AK000868 | Hs.5570 | hypothetical protein FLJ10006 | 1.4 | 5.2 |
| 132765 | BE222975 | Hs.56205 | insulin induced gene 1 | 1.1 | 5.8 |
| 132782 | F07424 | Hs.279840 | zinc finger protein 222 | 1.3 | 3.7 |
| 132793 | AB020713 | Hs.56966 | KIAA0906 protein | 2.3 | 6.3 |
| 132805 | AW975748 | Hs.5724 | sclerostin | 0.7 | 7.7 |
| 132863 | BE268048 | Hs.236494 | RAB10, member RAS oncogene family | 1.8 | 6.2 |
| 132894 | D63209 | Hs.5944 | solute carrier family 11 (proton-coupled | 1.5 | 20.8 |
| 132930 | AA579258 | Hs.6083 | Homo sapiens cDNA: FLJ21028 fis, clone C | 1 | 3.8 |
| 132932 | AW118826 | Hs.6093 | Homo sapiens cDNA: FLJ22783 fis, clone K | 0.7 | 5.4 |
| 132933 | BE263252 | Hs.6101 | hypothetical protein MGC3178 | 1.6 | 4.1 |
| 132965 | AI248173 | Hs.191460 | hypothetical protein MGC12936 | 1 | 4.2 |
| 132984 | BE539199 | Hs.62112 | zinc finger protein 207 | 1.5 | 4.4 |
| 132990 | X77343 | Hs.334334 | transcription factor AP-2 alpha (activat | 13.9 | 0.8 |
| 132998 | Y00062 | Hs.170121 | protein tyrosine phosphatase, receptor t | 0.6 | 4.6 |
| 133002 | AW499985 | Hs.42915 | ARP2 (actin-related protein 2, yeast) ho | 1.5 | 11.1 |
| 133011 | NM_006379 | Hs.171921 | sema domain, immunoglobulin domain (Ig), | 3.5 | 1 |
| 133012 | AA847843 | Hs.62711 | Homo sapiens, clone IMAGE: 3351295, mRNA | 1 | 4.5 |
| 133040 | AW502761 | Hs.30909 | KIAA0430 gene product | 0.9 | 5.5 |
| 133056 | H12028 | Hs.6396 | jumping translocation breakpoint | 1.7 | 5.3 |
| 133063 | AI654133 | Hs.30212 | thyroid receptor interacting protein 15 | 0.6 | 4.9 |
| 133067 | AK000708 | Hs.169764 | hypothetical protein FLJ20701 | 1.2 | 3.5 |
| 133080 | AF089816 | Hs.6454 | chromosome 19 open reading frame 3 | 1.2 | 17.5 |
| 133110 | AA808177 | Hs.65228 | ESTs | 0.9 | 5.1 |
| 133150 | AV655783 | Hs.661 | Empirically selected from AFFX single pr | 1.1 | 4.5 |
| 133175 | AW955632 | Hs.66666 | ESTs, Weakly similar to S19560 proline-r | 1.5 | 4.8 |
| 133199 | AF231981 | Hs.250175 | homolog of yeast long chain polyunsatura | 5.5 | 5.9 |
| 133203 | AA464362 | Hs.6748 | hypothetical protein PP1665 | 1.2 | 3.7 |
| 133206 | AB037773 | Hs.6762 | hypothetical protein | 1.6 | 8.6 |
| 133221 | W32474 | Hs.301746 | RAP2A, member of RAS oncogene family | 2.4 | 4.8 |
| 133229 | AL137480 | Hs.6834 | KIAA1014 protein | 1 | 4.2 |
| 133241 | AW796524 | Hs.68644 | Homo sapiens microsomal signal peptidase | 1.3 | 3.9 |
| 133257 | BE617892 | Hs.6895 | actin related protein 2/3 complex, subun | 1.4 | 5.4 |
| 133271 | Z48633 | Hs.283742 | H. sapiens mRNA for retrotransposon | 3.1 | 0.7 |
| 133273 | N27672 | Hs.69469 | dendritic cell protein | 2.5 | 6.5 |
| 133287 | AW797437 | Hs.69771 | B-factor, properdin | 1.3 | 4 |
| 133291 | BE297855 | Hs.69855 | NRAS-related gene | 1.4 | 5 |
| 133292 | AA304961 | Hs.699 | peptidylprolyl isomerase B (cyclophilin | 2.2 | 6.8 |
| 133294 | AJ001388 | Hs.69997 | zinc finger protein 238 | 1.5 | 4.3 |
| 133300 | AF116666 | Hs.70333 | hypothetical protein MGC10753 | 1.4 | 6.3 |
| 133302 | X04898 | Hs.237658 | apolipoprotein A-II | 0.2 | 3.6 |
| 133308 | U56979 | Hs.250651 | H factor 1 (complement) | 0.6 | 5 |
| 133347 | BE257758 | Hs.71475 | acid cluster protein 33 | 1.2 | 4.2 |
| 133370 | AF245505 | Hs.72157 | DKFZP564I1922 protein | 3.7 | 5.8 |
| 133404 | AB007916 | Hs.214646 | KIAA0447 gene product | 1.4 | 5.1 |
| 133408 | AI738719 | Hs.198427 | hexokinase 2 | 0.9 | 6.3 |
| 133422 | AB033061 | Hs.73287 | KIAA1235 protein | 1.2 | 3.7 |
| 133442 | AL137663 | Hs.7378 | Homo sapiens mRNA; cDNA DKFZp434G227 (fr | 0.7 | 4.8 |
| 133448 | M27749 | Hs.288168 | immunoglobulin lambda-like polypeptide 1 | 1.1 | 4.3 |
| 133449 | AF038962 | Hs.7381 | voltage-dependent anion channel 3 | 0.7 | 4.2 |
| 133501 | AI962602 | Hs.74284 | hypothetical protein MGC2714 | 3.1 | 5.9 |
| 133504 | NM_004415 | Hs.74316 | desmoplakin (DPI, DPII) | 4.3 | 11.5 |
| 133506 | BE562958 | Hs.74346 | hypothetical protein MGC14353 | 1.8 | 19.7 |
| 133532 | D87452 | Hs.74579 | KIAA0263 gene product | 1.2 | 5.4 |
| 133574 | H97991 | Hs.193313 | Empirically selected from AFFX single pr | 1.4 | 3.9 |
| 133586 | AI929645 | Hs.225936 | synapsin I | 0.8 | 4.9 |
| 133589 | L37368 | Hs.75104 | RNA-binding protein S1, serine-rich doma | 2 | 10.8 |
| 133591 | AI423369 | Hs.75111 | protease, serine, 11 (IGF binding) | 2.1 | 4.5 |
| 133606 | U10564 | Hs.75188 | wee1+ (S. pombe) homolog | 3.3 | 1.1 |
| 133617 | BE244334 | Hs.75249 | ADP-ribosylation factor-like 6 interacti | 2.3 | 5.6 |
| 133651 | AI301740 | Hs.173381 | dihydropyrimidinase-like 2 | 0.8 | 13.5 |
| 133660 | H14843 | Hs.303154 | popeye protein 3 | 1 | 9.1 |
| 133663 | AJ006239 | Hs.75438 | quinoid dihydropteridine reductase | 0.5 | 5.8 |
| 133668 | L77964 | Hs.271980 | mitogen-activated protein kinase 6 | 1.1 | 6.9 |
| 133671 | AW503116 | Hs.301819 | zinc finger protein 146 | 1.8 | 3.8 |
| 133681 | AI352558 | Hs.75544 | tyrosine 3-monooxygenase/tryptophan 5-mo | 1.5 | 11.1 |
| 133694 | W17187.comp | Hs.232400 | heterogeneous nuclear ribonucleoprotein | 2 | 3.9 |
| 133708 | AI018666 | Hs.75667 | synaptophysin | 0.6 | 3.5 |
| 133737 | AW001130 | Hs.75824 | KIAA0174 gene product | 1.2 | 7.2 |
| 133743 | AI929587 | Hs.75847 | CREBBP/EP300 inhibitory protein 1 | 1.5 | 5 |
| 133750 | BE410769 | Hs.75873 | zyxin | 1.2 | 4.8 |
| 133765 | M62194 | Hs.75929 | cadherin 11, type 2, OB-cadherin (osteob | 3.2 | 4.1 |
| 133776 | BE268649 | Hs.177766 | ADP-ribosyltransferase (NAD+; poly (ADP- | 2.1 | 3.8 |
| 133799 | W24087 | Hs.76285 | DKFZP564B167 protein | 1.9 | 12.6 |
| 133800 | AF075337 | Hs.76293 | thymosin, beta 10 | 2.6 | 6.6 |
| 133802 | AW239400 | Hs.76297 | G protein-coupled receptor kinase 6 | 1 | 4.9 |
| 133806 | D25969 | Hs.76325 | step II splicing factor SLU7 | 0.5 | 3.8 |
| 133817 | AW578716 | Hs.7644 | H1 histone family, member 2 | 1.5 | 4.5 |
| 133829 | AW630088 | Hs.76550 | Homo sapiens mRNA; cDNA DKFZp564B1264 (f | 3.7 | 6.6 |
| 133841 | AA345824 | Hs.76688 | carboxylesterase 1 (monocyte/macrophage | 0.3 | 4.4 |
| 133845 | AA147026 | Hs.76704 | ESTs | 5.5 | 2.9 |
| 133863 | AI815523 | Hs.76930 | synuclein, alpha (non A4 component of am | 0.6 | 4.8 |
| 133887 | X07767 | Hs.77271 | protein kinase, cAMP-dependent, catalyti | 1 | 10.2 |
| 133892 | AW859528 | Hs.301497 | arginyltransferase 1 | 0.9 | 4.8 |
| 133913 | AU076964 | Hs.7753 | calumenin | 2.8 | 10.5 |
| 133914 | AI458213 | Hs.77542 | ESTs | 1.8 | 5.6 |
| 133917 | AL031177 | Hs.7756 | proteasome (prosome, macropain) 26S subu | 1.5 | 6.6 |
| 133947 | L41066 | Hs.77810 | nuclear factor of activated T-cells, cyt | 1.5 | 3.8 |
| 133986 | M54968 | Hs.184050 | v-Ki-ras2 Kirsten rat sarcoma 2 viral on | 0.9 | 4.3 |
| 133987 | L15409 | Hs.174007 | von Hippel-Lindau syndrome | 2.3 | 4.3 |
| 133989 | AL040328 | Hs.78202 | SWI/SNF related, matrix associated, acti | 3.3 | 3.4 |
| 133990 | R48316 | Hs.7822 | Homo sapiens mRNA; cDNA DKFZp564C1216 (f | 1.3 | 5.7 |
| 134029 | BE150882 | Hs.143601 | hypothetical protein hCLA-iso | 1 | 6.5 |
| 134040 | NM_003470 | Hs.78683 | ubiquitin specific protease 7 (herpes vi | 1.7 | 3.6 |
| 134042 | AI027881 | Hs.7869 | lysosomal | 1 | 7.5 |
| 134049 | AF117236 | Hs.78825 | matrin 3 | 1.2 | 4 |
| 134095 | NM_004354 | Hs.79069 | cyclin G2 | 2.7 | 4.8 |
| 134098 | BE513171 | Hs.79086 | mitochondrial ribosomal protein L3 | 3.3 | 2.1 |
| 134207 | Z43039 | Hs.170198 | KIAA0009 gene product | 1.3 | 3.5 |
| 134210 | AF035606 | Hs.80019 | programmed cell death 6 | 1.7 | 6.9 |
| 134218 | U77735 | Hs.80205 | pim-2 oncogene | 0.8 | 5.3 |
| 134270 | X68194 | Hs.80919 | synaptophysin-like protein | 1.4 | 11.4 |
| 134277 | NM_004369 | Hs.80988 | collagen, type VI, alpha 3 | 2.6 | 3.5 |
| 134280 | NM_000712 | Hs.81029 | biliverdin reductase A | 1.8 | 5.8 |
| 134288 | AI022650 | Hs.8117 | erbb2-interacting protein ERBIN | 1.1 | 3.6 |
| 134296 | R00603 | Hs.8128 | phosphatidylserine decarboxylase | 1.1 | 5.9 |
| 134300 | NM_001430 | Hs.8136 | endothelial PAS domain protein 1 | 0.5 | 4.8 |
| 134310 | AL037800 | Hs.8148 | selenoprotein T | 1.7 | 7.9 |
| 134343 | D50683 | Hs.82028 | transforming growth factor, beta recepto | 0.8 | 7.6 |
| 134364 | X76534 | Hs.82226 | glycoprotein (transmembrane) nmb | 2.2 | 3.6 |
| 134374 | N22687 | Hs.8236 | ESTs | 1.9 | 3.6 |
| 134378 | AL035786 | Hs.82425 | actin related protein 2/3 complex, subun | 1.5 | 8.3 |
| 134382 | BE512856 | Hs.109051 | SH3 domain binding glutamic acid-rich pr | 1.1 | 3.6 |
| 134415 | AI750762 | Hs.82911 | protein tyrosine phosphatase type IVA, m | 1.9 | 4.6 |
| 134417 | NM_006416 | Hs.82921 | solute carrier family 35 (CMP-sialic aci | 1.2 | 7.5 |
| 134421 | AU077196 | Hs.82985 | collagen, type V, alpha 2 | 6.6 | 8.7 |
| 134439 | Z23024 | Hs.138860 | Rho GTPase activating protein 1 | 2 | 3.9 |
| 134454 | NM_013230 | Hs.286124 | CD24 antigen (small cell lung carcinoma | 3.5 | 1.1 |
| 134494 | D86981 | Hs.84084 | amyloid beta precursor protein (cytoplas | 1.5 | 4.4 |
| 134501 | W84869 | Hs.211568 | eukaryotic translation initiation factor | 1.2 | 5.7 |
| 134505 | AW960673 | Hs.177530 | ATP synthase, H+ transporting, mitochond | 1.3 | 3.9 |
| 134520 | BE091005 | Hs.74861 | activated RNA polymerase II transcriptio | 1.8 | 4.3 |
| 134528 | M23161 | Hs.84775 | Human transposon-like element mRNA | 0.8 | 5.6 |
| 134545 | AI902899 | Hs.85155 | butyrate response factor 1 (EGF-response | 1.4 | 5 |
| 134553 | AI203545 | Hs.296169 | S-phase response (cyclin-related) | 0.8 | 3.9 |
| 134573 | NM_016142 | Hs.279617 | steroid dehydrogenase homolog | 1.3 | 5.7 |
| 134576 | AB033017 | Hs.8594 | KIAA1191 protein | 0.9 | 3.7 |
| 134577 | BE244323 | Hs.85951 | exportin, tRNA (nuclear export receptor | 4 | 6.8 |
| 134579 | AW936928 | Hs.85963 | DKFZP564M182 protein | 2.2 | 4.3 |
| 134582 | AA927177 | Hs.86041 | CGG triplet repeat binding protein 1 | 1.6 | 3.6 |
| 134600 | AF078859 | Hs.86347 | hypothetical protein | 2.1 | 3.5 |
| 134655 | AF265208 | Hs.123090 | SWI/SNF related, matrix associated, acti | 1.7 | 4.2 |
| 134700 | AK000606 | Hs.8868 | golgi SNAP receptor complex member 1 | 4.4 | 0.9 |
| 134737 | D17530 | Hs.89434 | drebrin 1 | 3.1 | 1.6 |
| 134762 | T51986 | Hs.283108 | hemoglobin, gamma G | 0.5 | 4.6 |
| 134843 | AA428520 | Hs.90061 | progesterone binding protein | 1.3 | 3.7 |
| 134854 | J03464 | Hs.179573 | collagen, type I, alpha 2 | 8.7 | 17.3 |
| 134865 | AA587775 | Hs.66295 | multi-PDZ-domain-containing protein | 1.7 | 4 |
| 134868 | AB020689 | Hs.90419 | KIAA0882 protein | 3.4 | 0.9 |
| 134874 | AI803761 | Hs.90458 | serine palmitoyltransferase, long chain | 1.3 | 6.9 |
| 134885 | AJ002030 | Hs.9071 | progesterone membrane binding protein | 1.4 | 9.6 |
| 134891 | R51083 | Hs.90787 | ESTs | 1 | 10.1 |
| 134908 | BE089782 | Hs.9877 | hypothetical protein | 1.9 | 3.9 |
| 134934 | AF005043 | Hs.91390 | poly (ADP-ribose) glycohydrolase | 1 | 4.3 |
| 134970 | BE560779 | Hs.284233 | NICE-5 protein | 1.4 | 10.4 |
| 134982 | AK002085 | Hs.92308 | Homo sapiens cDNA FLJ11223 fis, clone PL | 1.6 | 4.1 |
| 135011 | AB037835 | Hs.92991 | KIAA1414 protein | 1.2 | 5.6 |
| 135032 | AW301984 | Hs.173685 | hypothetical protein FLJ12619 | 1.7 | 7.6 |
| 135035 | AL034344 | Hs.284186 | forkhead box C1 | 3.2 | 0.6 |
| 135051 | AI272141 | Hs.83484 | SRY (sex determining region Y)-box 4 | 4.2 | 4.1 |
| 135060 | AK001887 | Hs.259842 | protein kinase, AMP-activated, gamma 2 n | 1.3 | 4.8 |
| 135062 | AK000967 | Hs.93872 | KIAA1682 protein | 2 | 3.7 |
| 135077 | AW503733 | Hs.9414 | KIAA1488 protein | 2.8 | 3.7 |
| 135082 | AB017363 | Hs.94234 | frizzled (Drosophila) homolog 1 | 2.4 | 4.8 |
| 135107 | T97257 | Hs.337531 | ESTs, Moderately similar to I38022 hypot | 1.4 | 5.8 |
| 135143 | AA132813 | Hs.69559 | KIAA1096 protein | 1.8 | 8.5 |
| 135156 | BE563088 | Hs.9552 | binder of ArI Two | 1.2 | 6.8 |
| 135172 | AB028956 | Hs.12144 | KIAA1033 protein | 3.1 | 1.4 |
| 135181 | BE250865 | Hs.279529 | px19-like protein | 1.3 | 7.5 |
| 135222 | AA534009 | Hs.183487 | interferon stimulated gene (20 kD) | 1.3 | 3.8 |
| 135232 | AL038812 | Hs.96800 | ESTs, Moderately similar to ALU7_HUMAN A | 2.1 | 3.9 |
| 135289 | AW372569 | Hs.9788 | hypothetical protein MGC10924 similar to | 0.9 | 8.4 |
| 135290 | AA331901 | Hs.184736 | hypothetical protein FLJ10097 | 1 | 3.8 |
| 135291 | T83882 | Hs.97927 | ESTs | 1.2 | 3.5 |
| 135349 | AA114212 | Hs.9930 | serine (or cysteine) proteinase inhibito | 2.6 | 8.9 |
| 135357 | AI565004 | Hs.79572 | cathepsin D (lysosomal aspartyl protease | 2.5 | 5.4 |
| 135398 | M16029 | Hs.287270 | ret proto-oncogene (multiple endocrine n | 0.4 | 7.9 |
| 135399 | W79431 | Hs.326249 | ribosomal protein L22 | 1.5 | 4.5 |
| 135400 | X78592 | Hs.99915 | androgen receptor (dihydrotestosterone r | 3.2 | 1.8 |
| 302665 | R99693 | Hs.224410 | Homo sapiens cDNA FLJ12843 fis, clone NT | 3.6 | 3.6 |
| 302892 | AW176909 | Hs.42346 | calcineurin-binding protein calsarcin-1 | 3.3 | 1.6 |
| 302963 | AW673106 | Hs.151945 | mitochondrial ribosomal protein L43 | 0.9 | 4.2 |
| 303131 | AW081061 | Hs.103180 | DC2 protein | 3 | 17.3 |
| 303150 | AA887146 | Hs.8217 | stromal antigen 2 | 6.2 | 4 |
| 310125 | AA147979 | Hs.285005 | mitochondrial import receptor Tom22 | 1.2 | 6.6 |
| 312662 | AA233808 | Hs.286241 | protein kinase, cAMP-dependent, regulato | 1 | 3.5 |
| 319429 | BE616412 | Hs.286218 | junctional adhesion molecule 1 | 1.5 | 4.7 |
| 320591 | AA054761 | Hs.169149 | karyopherin alpha 1 (importin alpha 5) | 1.2 | 5.6 |
| 406779 | AA412048 | Hs.279574 | CGI-39 protein; cell death-regulatory pr | 1.3 | 3.5 |
| 410691 | AW239226 | Hs.65450 | reticulon 4 | 1.2 | 13.9 |
| 410763 | AF279145 | Hs.8966 | hypothetical protein FLJ21776 | 2 | 5.1 |
| 415738 | BE539367 | Hs.295953 | ESTs, Weakly similar to AF220049 1 uncha | 1.3 | 3.9 |
| 420186 | NM_015925 | Hs.95697 | liver-specific bHLH-Zip transcription fa | 1.5 | 6.2 |
| 422055 | NM_014320 | Hs.111029 | putative heme-binding protein | 2 | 11.3 |
| 425815 | R94023 | Hs.337531 | ESTs, Moderately similar to I38022 hypot | 1.7 | 3.6 |
| 426218 | AF119043 | Hs.168005 | Homo sapiens cDNA FLJ13372 fis, clone PL | 3.3 | 2.8 |
| 427397 | AI929685 | Hs.177656 | calmodulin 1 (phosphorylase kinase, delt | 1.3 | 4.7 |
| 427466 | AA523543 | Hs.7678 | cellular retinoic acid-binding protein 1 | 1.1 | 3.7 |
| 427505 | AA361562 | Hs.178761 | 26S proteasome-associated pad1 homolog | 3.2 | 2.5 |
| 427723 | AI355260 | Hs.279789 | histone deacetylase 3 | 2.8 | 22 |
| 428673 | AW601325 | Hs.324278 | Homo sapiens mRNA; cDNA DKFZp566M063 (fr | 1.1 | 5.2 |
| 430219 | X99209 | Hs.235887 | HMT1 (hnRNP methyltransferase, S. cerevi | 1.8 | 8.8 |
| 430450 | R23553 | Hs.241489 | hypothetical protein | 1.1 | 5.6 |
| 432866 | BE395875 | Hs.279609 | mitochondrial carrier homolog 2 | 1.5 | 6.1 |
| 433423 | BE407127 | Hs.8997 | heat shock 70 kD protein 1A | 1.3 | 7.6 |
| 437562 | AB001636 | Hs.5683 | DEAD/H (Asp-Glu-Ala-Asp/His) box polypep | 1.6 | 6.5 |
| 437667 | BE616412 | Hs.286218 | junctional adhesion molecule 1 | 1.3 | 3.5 |
| 437754 | R60366 | Hs.5822 | Homo sapiens cDNA: FLJ22120 fis, clone H | 2 | 5.7 |
| 440252 | BE513940 | Hs.6101 | hypothetical protein MGC3178 | 1.1 | 6.2 |
| 441471 | AL042986 | Hs.7857 | erythrocyte membrane protein band | 0.5 | 3.7 |
| 448292 | BE281316 | Hs.47334 | hypothetical protein FLJ14495 | 2.5 | 4.9 |
| 449404 | H51066 | Hs.23581 | leptin receptor gene-related protein | 1.1 | 3.6 |
| 449964 | AW001741 | Hs.273193 | hypothetical protein FLJ10706 | 1.4 | 3.5 |
| 451389 | N73222 | Hs.279009 | matrix Gla protein | 4 | 11.2 |
| 452685 | AI634651 | Hs.30250 | v-maf musculoaponeurotic fibrosarcoma (a | 0.8 | 5.6 |
| | RC_H15847_s | | peptidylprolyl isomerase B (cyclophilin B) | 1.8 | 4.8 |
| | RC_W84712 | | calumenin | 3.5 | 4.6 |
| | X14008_ma1_f | | lysozyme (renal amyloidosis) | 0.9 | 4.5 |
| | RC_H86543_f | | ESTs | 1.8 | 6.6 |
| | H07011 | | ESTs; Weakly similar to SAS [H. sapiens] | 1.8 | 3.9 |
| | RC_AA164586_s | | ESTs | 6.2 | 0.8 |
| | RC_AA070485 | | Homo sapiens clone 23967 | 3.4 | 2.6 |
| | RC_H98714_s | | ESTs | 1.6 | 3.5 |
| | RC_AA406145_f | | ESTs | 4.6 | 3 |
| | AA458584 | | SRY (sex determining region Y)-box 4 | 3.4 | 0.4 |
| | AA031548 | | cell division cycle 42 (GTP-binding protein; 25 kD) | 3.1 | 3.9 |
| | X02761 | | fibronectin 1 | 3.6 | 15.2 |
| | RC_AA487193 | | secreted frizzled-related protein 4 | 4.7 | 4 |
| | R25326 | | Homo sapiens mRNA for putative vacuolar | 0.9 | 5 |
| | RC_AA393805 | | ESTs; Weakly similar to (define not | 1.1 | 8.4 |
| | RC_AA449333 | | ESTs | 2.9 | 4.6 |
| | RC_AA287681_s | | ESTs | 1.3 | 4 |
| | RC_AA490864 | | ESTs; Highly similar to heat shock factor | 1.4 | 5 |
| | RC_C14243_f | | ESTs; Highly similar to heat shock factor | 1.7 | 5 |
| | R21443 | | ESTs | 1.6 | 3.7 |
| | RC_AA251902 | | Homo sapiens lysophospholipase (LPL1) | 2.2 | 3.8 |
| | M21121_s | | small inducible cytokine A5 (RANTES) | 0.9 | 9.9 |
| | C00038_s | | ESTs | 2.8 | 4.8 |
| | Y00503 | | keratin 19 | 3.1 | 1.1 |
| | RC_R27006_f | | ESTs | 1.6 | 3.7 |
| | RC_AA416886 | | ESTs; Weakly similar to predicted using | 3.1 | 3.1 |
| | RC_AA460450 | | fibroblast growth factor receptor 2 (bacteria- | 1.5 | 3.7 |
| | RC_AA488433 | | ESTs; Weakly similar to deduced amino acid | 1.1 | 4 |
| | RC_AA278400_f | | Home sapiens HRIHFB2115 mRNA; partial cds | 1.5 | 3.6 |
| | U28831 | | Human protein immuno-reactive with anti-PTH | 4.4 | 0.6 |
| | RC_AA199588 | | Homo sapiens actin-related protein Arp3 (ARP3) | 1.8 | 4.7 |
| | AF006082 | | Homo sapiens actin-related protein Arp2 (ARP2) | 1.6 | 10.9 |
| | RC_H90899 | | desmoplakin (DPI; DPII) | 5.4 | 5.5 |
| | RC_W95070 | | desmoplakin (DPI; DPII) | 5 | 2.6 |
| | RC_T90946_f | | Human mRNA for KIAA263 gene; complete cds | 1.1 | 3.9 |
| | D87258 | | protease; serine; 11 (IGF binding) | 2.4 | 3.5 |
| | AA313414_s | | ESTs; Weakly similar to cDNA EST EMBL: T1157 | 1.5 | 5.3 |
| | RC_H73484_s | | ESTs; Weakly similar to similar to Yeast | 1.3 | 6.3 |
| | AFFX-HUMISGF3A/ | | | 2.3 | 13.5 |
| | M97935_3 | | | | |
| | AFFX-HUMRGE/ | | | 1.1 | 7.9 |
| | M10098_5 | | | | |
| | AFFX-M27830_5 | | | 0.5 | 7.4 |
| | AFFX-M27830_5 | | | 0.6 | 5.4 |
| | RC_AA063431_f | | ESTs | 0.8 | 4.1 |
| | RC_T63769_f | | ferritin; light polypeptide | 1.1 | 3.7 |

Table 8 shows genes upregulated in tumor tissue compared to normal breast tissue. Specifically, one column shows the ratio of expression of the indicated gene in breast tumor tissue compared to other body tissues, and another column shows the ratio of expression of the indicated gene in breast tumor tissue compared to normal breast tissue.
Pkey: Unique Eos probeset identifier number
ExAccn: Exemplar Accession number, Genbank accession number
UnigeneID: Unigene number
Unigene Title: Unigene gene title
R1: Ratio of tumor to normal body tissue
R2: Ratio of tumor to normal breast tissue

TABLE 8A
Table 8A shows the accession numbers for those pkeys lacking unigeneID's for Table 8. For
each probeset, we have listed the gene cluster number from which the oligonucleotides were
designed. Gene clusters were compiled using sequences derived from Genbank ESTs and
mRNAs. These sequences were clustered based on sequence similarity using Clustering and
Alignment Tools (DoubleTwist, Oakland California). The Genbank accession numbers for
sequences comprising each cluster are listed in the “Accession” column.

| | |
|---|---|
| Pkey: | Unique Eos probeset identifier number |
| CAT number: | Gene cluster number |
| Accession: | Genbank accession numbers |

| Pkey | CAT number | Accessions |
|---|---|---|
| 108469 | 116761_1 | AA079487 AA128547 AA128291 AA079587 AA079600 |
| 125076 | 190299_1 | AA973971 T88817 AA253263 |
| 114636 | 109698_1 | AA075488 AA129081 AA074851 AA082852 AA074732 AA084908 AA084751 AA076042 AA131172 AA085374 AA079519 |
| | | AA074510 AA113824 AA102437 AA070833 AA070143 AA084693 AA084389 AA076373 AA075492 AA062834 AA084335 |
| | | AA078829 AA079344 AA069916 AA079275 AA070914 AA654069 AA081976 AA080957 AA083115 AA070942 AA085296 |
| 123526 | genbank_AA608657 | AA608657 |
| 123533 | genbank_AA608751 | AA608751 |
| 125090 | genbank_T91518 | T91518 |
| 125154 | genbank_W38419 | W38419 |
| 118475 | genbank_N66845 | N66845 |
| 118505 | genbank_N67343 | N67343 |
| 101046 | entrez_K01160 | K01160 |
| 129982 | 221_267 | Z14221 AW381862 M97920 AW401444 Z66542 M29470 AW406502 X61011 M34024 AA327072 Z14166 Z14167 Z14165 |
| | | AW403806 Z14200 AA383972 Z14205 Z14201 M18513 Z14202 AW403684 X14584 AF062221 U43760 X65892 X65883 X62107 |
| | | Z80847 X65885 X65893 AF062142 X65891 X17675 Z47274 Z47277 Z47276 X65888 Z47275 X62109 AF062140 L01278 |
| | | AF062134 AF062139 X81723 Z80840 X81733 X81743 X81744 X81732 Z80843 AW402942 AW403516 X65919 AF062190 |
| | | AF062177 AF062222 AF062115 Z47240 AF062263 AF062261 AF062223 AF062211 Z47238 AW401714 AW404008 AW404991 |
| | | AF062280 M99595 Z47214 Z47232 Z47218 M26995 AF062184 X65895 L38433 X81731 Z11946 Z47226 AF062205 AF174012 |
| | | L01276 AF062168 AF062136 X81755 X81748 AF174019 AF062285 L06924 L11699 AW402665 L09070 L28049 L08090 |
| | | AW407843 Z14171 AW402944 L08083 AW405627 L33035 L26907 M17750 L28052 M17751 AJ239360 U19885 L14821 X56526 |
| | | AB014341 L12087 L12098 U68231 L12184 AF062242 AF022000 U64499 U00570 AI268604 Y15773 X64239 X62969 U00506 |
| | | X73605 X99360 U00577 D83677 AB021539 AF035796 Z33899 U00588 AJ239353 AF174062 Z33901 X98899 AF174058 X63080 |
| | | D83676 AB021529 Z18318 U00488 L01412 X81746 U21262 U21272 U00560 AF174060 U00547 U00561 Z18321 S73957 |
| | | M26435 AF115130 AF115117 Z92896 U21254 Z92895 AF115112 AF062290 T28938 Y09386 AF174067 U27189 Y09384 |
| | | U77373 AF174057 M17749 X69692 AF174038 U64478 U64486 AF174098 X99365 AF174077 AF174083 AF174089 AF015133 |
| | | AF127792 AJ006171 U00510 U58144 AF004323 AF115109 AF004324 AF015130 AF090414 AF090418 AB021536 AB021519 |
| | | Z96957 AF021986 Z92898 Z96956 L34164 AF062251 AF052524 U00549 U00541 083687 AF052522 AF087420 AF052520 |
| | | AF022011 L43085 U00536 L15515 L43083 AF090420 AB021530 AB021534 Z96955 Z96954 Z33903 X62964 AF103282 U00509 |
| | | AF062298 AF062289 AF062307 AW408326 L33037 L04337 L04328 X81742 L04336 L03385 AF032360 S56184 AF062191 |
| | | AF191092 AI906954 AF103184 L04343 AW364860 AF001424 AF103163 Z98717 AF103143 AJ008250 L04323 AF103321 |
| | | AF103335 L04342 L03818 L03817 AW404978 AW403690 U86801 AF103150 AJ010334 AF035027 AJ007327 AF103115 |
| | | AF017458 AJ008207 AJ008183 AJ008196 AJ008241 AJ008208 AF103210 AF068668 AF068670 AF068671 AF068664 |
| | | AF068669 AF068666 AF068665 AF068672 AW371244 AW403670 AW408074 AW404575 AW362153 AW403803 AW406702 |
| | | AW351514 D78345 T29140 J00231 NM_002179 AW405146 AA301091 X04646 H64660 AW402990 AW406534 T93007 |
| | | AI857980 AW368899 AI905833 AW406586 AA482084 AI872299 AA715266 AW404328 AI831674 AI709348 AA603112 |
| | | AW514864 AA485775 H64492 AW404789 AA487630 AA715498 AA295885 T27613 T98113 |
| 108470 | genbank_AA079500 | AA079500 |
| 101447 | entrez_M21305 | M21305 |
| 124447 | genbank_N48000 | N48000 |
| 101624 | entrez_M55998 | M55998 |
| 131791 | 221_260 | X62111 S67984 AJ131056 Z47243 Z47235 AF062268 Z47237 AJ131058 AF062130 AF062282 X62108 AA385989 AA464794 |
| | | X69861 AW402964 M90808 Z98735 Z98734 Z98736 AF035018 X79161 U00545 AF174046 AF174071 U00552 U96288 |
| | | AF021989 AF062255 AF174061 S73953 AF062135 AF062155 X64147 U00555 AF174070 AF129754 U86789 Z98714 Z98738 |
| | | S75886 AJ008175 AJ008186 AJ008188 Y08403 H15753 H22208 AA327241 AW405737 H42300 H39677 H26239 H26903 |
| | | H45128 R86072 AA327565 AI660584 AW361537 AW383759 S71043 H15014 H45570 H42819 H45523 H45134 R72043 |
| | | H24543 H27636 H27610 T28147 H25496 AW364071 AW364072 H45561 H45556 H42605 AI735017 T47421 R46719 H27570 |
| | | H44599 AI459598 H42347 H41938 H24993 AA345888 H22339 AI538691 AJ012264 AA664201 AI880450 AA327310 AI991250 |
| | | AI633028 AW001210 AI956075 H30467 PA326915 H41943 AI749266 AI744441 AA327377 AW512326 AI735170 H01634 |
| | | AI587047 AI571623 AA327486 AA327103 AA327195 AA326973 T28143 |
| 124842 | 217726_1 | R56485 R37248 R59992 |
| 103758 | AA084874_f_at | AA084874_f |
| 130064 | 221_264 | X57815 L29157 AA367448 AW328098 AW404536 L29156 L29163 H27683 R83195 AA295096 AW327822 H25458 AW404692 |
| | | X57819 X57823 AW405604 AW404447 Z34914 AW406542 AA427726 AA604389 AW405606 AW405918 AW405117 |
| | | AW407182 L03632 AW405058 L03627 AW407470 R72736 L21959 AW375738 X87888 AF124166 AF054638 AJ241406 |
| | | U09902 AW405284 M12025 PA360219 L03631 L03629 AW327727 AF194810 AF058076 AF194686 AF063774 AF063737 |
| | | AF063755 AF063740 AF063739 AF063738 S73129 L43088 AF063775 L33026 AF060136 AF194813 AF194812 AF194811 |
| | | AF194809 AF194814 AF194808 AF064505 AF064503 AF063765 AF063757 AF063752 AF063716 AF063736 AF194806 |
| | | AF058077 AF063747 AF063772 AF063781 AF060137 AF194805 AF060134 AF060132 AF058074 AF063754 AF063704 |
| | | U38589 Z18332 AF060122 AF194807 AF060135 AF064506 AF064504 AF063773 L26540 AF058072 AF060131 AF064500 |
| | | AF060136 AF064513 AF194683 AF194711 AF058075 AF063717 Z19546 AF194581 X72746 U96393 U09890 X98897 |
| | | AF194592 M80916 AJ241405 AF194632 AJ388659 AF194625 Z74694 AF194588 AF194601 U09901 U09911 U09892 |
| | | AJ010336 AJ006162 AJ249377 AJ241414 AA327392 U97248 X72747 Z46850 X95739 AW406701 AJ243109 AF194609 |
| | | S80758 AF194595 AF194596 L22483 Z70262 D84141 AW405758 AF001788 AF194580 D84143 U76684 AF194593 L03630 |
| | | X87892 X91134 U21249 Z46346 AJ132426 AF103659 AJ233718 AF021038 AJ233727 H24657 U09882 S75627 AA573599 |
| | | AF047231 AF047232 AF103616 AW404484 Z46848 S76132 AF103663 AF103713 Y17940 AF047216 AF103595 Y17956 |
| | | AF052799 AF052797 AF052802 AF052798 AF052801 AF052794 AF052796 AF047218 AF052800 AF047217 AF052795 |
| | | AF032351 AF103701 AF103708 AF103710 AF103706 S62232 AF103645 AF103632 AF103647 AF103644 AF103640 |
| | | AF103692 AF093581 AF103620 AW405934 AI445389 AW383753 AA360256 AF099676 H21654 H39501 AI820828 H53689 |
| | | W26785 AW384496 AW407708 AA541663 AA911602 AI821461 AA588300 AA327050 H42717 AI951280 AA421322 AI923193 |
| | | AA864302 H25133 D87023 J03011 M61771 D87017 AA526865 AA253450 |
| 130232 | 18831_2 | U29463 U06715 W24970 AA584303 T39581 AA155603 AA305043 AA429426 W05664 AA102382 AA482044 W24487 AA319060 |
| | | T88946 F10106 AA232161 AA243117 AA158937 AA100864 |
| 109097 | genbank_AA167512 | AA167512 |

TABLE 9
FIG. 9 from BRCA 001-2 US

Table 9 depicts a preferred group of genes upregulated in tumor tissue
compared to normal breast tissue.

| | |
|---|---|
| Pkey: | Unique Eos probeset identifier number |
| ExAccn: | Exemplar Accession number, Genbank accession number |
| UnigeneID: | Unigene number |
| Unigene Title: | Unigene gene title |

| Pkey | ExAccn | UnigeneID | UnigeneTitle |
|---|---|---|---|
| 100690 | AA383256 | Hs.1657 | estrogen receptor 1 |
| 102211 | BE314524 | Hs.78776 | putative transmembrane protein |
| 103587 | BE270266 | Hs.82128 | 5T4 oncofetal trophoblast |
| | | | glycoprotein |
| 104115 | AF183810 | Hs.26102 | opposite strand to trichorhino- |
| | | | phalangeal syndrome I |
| 105038 | AW503733 | Hs.9414 | KIAA1488 protein |
| 105500 | AW602166 | Hs.222399 | CEGP1 protein |
| 105990 | AI690586 | Hs.29403 | hypothetical protein FLJ22060 |
| 106155 | AA425414 | Hs.33287 | nuclear factor I/B |
| 106373 | AW503807 | Hs.21907 | histone acetyltransferase |
| 106414 | BE568205 | Hs.28827 | mitogen-activated protein kinase |
| | | | kinase kinase 2 |
| 110009 | BE075297 | Hs.6614 | ESTs, Weakly similar to A43932 |
| | | | mucin 2 precursor, intestinal |
| 111900 | AF131784 | Hs.25318 | Homo sapiens clone 25194 mRNA |
| | | | sequence |
| 114540 | AI904232 | Hs.75323 | prohibitin |
| 116470 | AI272141 | Hs.83484 | SRY (sex determining region |
| | | | Y)-box 4 |
| 117280 | M18217 | Hs.172129 | Homo sapiens cDNA: FLJ21409 |
| | | | fis, done COL03924 |
| 119771 | AI905687 | Hs.2533 | EST |
| 121723 | AA243499 | Hs.104800 | hypothetical protein FLJ10134 |
| 124059 | BE387335 | Hs.283713 | ESTs, Weakly similar to S64054 |
| | | | hypothetical protein YGL050w |
| 131148 | AW953575 | Hs.303125 | p53-induced protein PIGPC1 |
| 132371 | AA235448 | Hs.46677 | PRO2000 protein |
| 134169 | AI690916 | Hs.178137 | transducer of ERBB2, 1 |
| 302235 | AL049987 | Hs.166361 | Homo sapiens mRNA; cDNA |
| | | | DKFZp564F112 |
| 452410 | AL133619 | Hs.29383 | Homo sapiens mRNA; cDNA |
| | | | DKFZp434 |

TABLE 10
FIG. 10 from BRCA 001-3 PCT

| Pkey | ExAccn | UnigeneID | Unigene Title | R1 | R2 | R3 | R4 |
|---|---|---|---|---|---|---|---|
| 100082 | AA130080 | Hs.4295 | proteasome (prosome, macropain) 26S subu | 4.2 | 152 | 36 | 12.2 |
| 100103 | AA380887 | Hs.5085 | dolichyl-phosphate mannosyltransferase p | 9.8 | 123 | 13 | 5 |
| 100131 | D12485 | Hs.11951 | ectonucleotide pyrophosphatase/phosphodi | 13.2 | 244 | 19 | 9.9 |
| 100147 | D13666 | Hs.136348 | osteoblast specific factor 2 (fasciclin | 15.7 | 1030 | 66 | 5 |
| 100154 | H60720 | Hs.81892 | KIAA0101 gene product | 4.1 | 320 | 78 | 10.6 |
| 100157 | D14661 | Hs.119 | Wilms' tumor 1-associating protein | 4.7 | 119 | 26 | 3 |
| 100169 | AL037228 | Hs.82043 | D123 gene product | 5.1 | 106 | 21 | 9.2 |
| 100203 | BE242284 | Hs.172199 | adenylate cyclase 7 | 4.7 | 47 | 1 | 4.3 |
| 100210 | D26361 | Hs.3104 | KIAA0042 gene product | 4.7 | 47 | 4 | 0.7 |
| 100219 | AW972300 | Hs.118110 | bone marrow stromal cell antigen 2 | 3.8 | 350 | 93 | 1.9 |
| 100234 | D29677 | Hs.3085 | KIAA0054 gene product; Helicase | 4.1 | 64 | 16 | 3 |
| 100248 | NM_015156 | Hs.78398 | KIAA0071 protein | 3.4 | 77 | 23 | 5.9 |
| 100252 | NM_006207 | Hs.170040 | platelet-derived growth factor receptor- | 4.5 | 45 | 4 | 4 |
| 100260 | D38491 | Hs.322478 | KIAA0117 protein | 5.9 | 59 | 1 | 2.6 |
| 100279 | D42084 | Hs.82007 | KIAA0094 protein | 3.5 | 96 | 28 | 1.3 |
| 100286 | BE247550 | Hs.86859 | growth factor receptor-bound protein 7 | 3.1 | 306 | 98 | 1.5 |
| 100294 | AA331881 | Hs.75454 | peroxiredoxin 3 | 12.8 | 128 | 1 | 11.7 |
| 100335 | AW247529 | Hs.6793 | platelet-activating factor acetylhydrola | 4.2 | 187 | 44 | 5.4 |
| 100365 | AI878927 | Hs.79284 | mesoderm specific transcript (mouse) hom | 4.5 | 129 | 29 | 3.1 |
| 100375 | D80004 | Hs.75909 | KIAA0182 protein | 3.5 | 78 | 23 | 4.8 |
| 100409 | D86957 | Hs.80712 | KIAA0202 protein | 10.2 | 102 | 1 | 4.8 |
| 100410 | D86961 | Hs.79299 | lipoma HMGIC fusion partner-like 2 | 4 | 40 | 1 | 3.8 |
| 100414 | NM_014735 | Hs.82292 | KIAA0215 gene product | 3.2 | 32 | 2 | 2.9 |
| 100418 | D86978 | Hs.84790 | KIAA0225 protein | 3.6 | 36 | 7 | 3.2 |
| 100438 | AA013051 | Hs.91417 | topoisomerase (DNA) II binding protein | 5.6 | 76 | 14 | 2 |
| 100439 | AA347720 | Hs.122669 | KIAA0264 protein | 3.5 | 35 | 9 | 3.1 |
| 100448 | AF234887 | Hs.57652 | cadherin, EGF LAG seven-pass G-type rece | 5.5 | 145 | 27 | 2.2 |
| 100449 | D87470 | Hs.75400 | KIAA0280 protein | 3.4 | 34 | 1 | 1.2 |
| 100522 | X51501 | Hs.99949 | prolactin-induced protein | 22.7 | 760 | 34 | 1.4 |
| 100552 | AA019521 | Hs.301946 | lysosomal | 14.4 | 144 | 9 | 4.7 |
| 100643 | NM_005032 | Hs.4114 | plastin 3 (T isoform) | 4.1 | 259 | 63 | 1.9 |
| 100661 | BE623001 | Hs.132748 | Homo sapiens ribosomal protein L39 mRNA, | 3.3 | 116 | 36 | 2.2 |
| 100666 | L05424 | Hs.169610 | CD44 antigen (homing function and Indian | 8.5 | 85 | 1 | 3.2 |
| 100667 | L05424 | Hs.169610 | CD44 antigen (homing function and Indian | 3 | 594 | 201 | 2.3 |
| 100745 | BE207168 | Hs.144630 | nuclear receptor subfamily 2, group F, m | 5 | 82 | 17 | 0.9 |
| 100774 | J05581 | Hs.89603 | mucin 1, transmembrane | 3.5 | 37 | 11 | 2.8 |
| 100783 | AF078847 | Hs.191356 | general transcription factor IIH, polype | 9.7 | 97 | 10 | 7.2 |
| 100821 | M26460 | | gb: Homo sapiens (clone 104) retinoblasto | 3.3 | 33 | 1 | 0.8 |
| 100864 | BE563957 | Hs.74861 | activated RNA polymerase II transcriptio | 3.7 | 477 | 130 | 3.1 |
| 100877 | X80821 | Hs.27973 | KIAA0874 protein | 6.3 | 63 | 4 | 5.7 |
| 100892 | BE245294 | Hs.180789 | S164 protein | 4.7 | 47 | 1 | 4.2 |
| 101038 | BE297139 | Hs.79411 | replication protein A2 (32 kD) | 3.8 | 115 | 30 | 7.1 |
| 101046 | K01160 | | NM_002122: Homo sapiens major histocompat | 3.9 | 390 | 100 | 11.1 |
| 101079 | BE264901 | Hs.250502 | carbonic anhydrase VIII | 3.9 | 39 | 8 | 3.6 |
| 101084 | AW409934 | Hs.75528 | nucleolar GTPase | 4.1 | 53 | 13 | 4 |
| 101104 | AW862258 | Hs.169266 | neuropeptide Y receptor Y1 | 15.3 | 153 | 1 | 14.1 |
| 101185 | NM_001621 | Hs.170087 | aryl hydrocarbon receptor | 11.3 | 113 | 8 | 3.9 |
| 101188 | L20320 | Hs.184298 | cyclin-dependent kinase 7 (homolog of Xe | 3.1 | 118 | 38 | 2 |
| 101201 | L22524 | Hs.2256 | matrix metalloproteinase 7 (MMP7; uterin | 8.2 | 396 | 48 | 0.9 |
| 101232 | AU077288 | Hs.242894 | ADP-ribosylation factor-like 1 | 4 | 110 | 28 | 10.7 |
| 101275 | BE545277 | Hs.3273 | Ts translation elongation factor, mitoch | 4.2 | 50 | 12 | 4.4 |
| 101300 | BE535511 | Hs.74137 | transmembrane trafficking protein | 6.6 | 135 | 21 | 13.1 |
| 101396 | BE267931 | Hs.78996 | proliferating cell nuclear antigen | 6.4 | 249 | 39 | 22.4 |
| 101447 | M21305 | | gb: Human alpha satellite and satellite 3 | 6.5 | 878 | 135 | 0.8 |
| 101448 | NM_000424 | Hs.195850 | keratin 5 (epidermolysis bullosa simplex | 4.8 | 622 | 130 | 0.7 |
| 101470 | NM_000546 | Hs.1846 | tumor protein p53 (Li-Fraumeni syndrome) | 5.1 | 97 | 19 | 9.3 |
| 101478 | NM_002890 | Hs.758 | RAS p21 protein activator (GTPase activa | 9.6 | 96 | 1 | 8.5 |
| 101484 | AA053486 | Hs.20315 | interferon-induced protein with tetratri | 11.2 | 112 | 8 | 5.9 |
| 101507 | X16896 | Hs.82112 | interleukin 1 receptor, type I | 3.9 | 39 | 2 | 3.5 |
| 101621 | BE391804 | Hs.62661 | guanylate binding protein 1, interferon- | 3.6 | 36 | 1 | 2.6 |
| 101624 | M55998 | | gb: Human alpha-1 collagen type I gene, 3 | 3.1 | 2898 | 923 | 2.2 |
| 101664 | AA436989 | Hs.121017 | H2A histone family, member A | 6.9 | 103 | 15 | 8.4 |
| 101684 | M63256 | Hs.75124 | cerebellar degeneration-related protein | 6.4 | 64 | 2 | 4.9 |
| 101724 | L11690 | Hs.620 | bullous pemphigoid antigen 1 (230/240 kD) | 9.4 | 94 | 1 | 0.3 |
| 101754 | S70114 | Hs.239489 | TIA1 cytotoxic granule-associated RNA-bi | 8.9 | 89 | 5 | 8 |
| 101767 | M81057 | Hs.180884 | carboxypeptidase B1 (tissue) | 3.6 | 824 | 227 | 1.4 |
| 101791 | M83822 | Hs.62354 | cell division cycle 4-like | 9 | 144 | 16 | 13 |
| 101794 | M84605 | Hs.957 | putative opioid receptor, neuromedin K ( | 3.3 | 36 | 11 | 2.4 |
| 101803 | AW024390 | Hs.155691 | pre-B-cell leukemia transcription factor | 5.4 | 180 | 34 | 15.9 |
| 101809 | M86849 | Hs.323733 | gap junction protein, beta 2, 26 kD (conn | 12 | 120 | 8 | 9 |
| 101839 | AA446644 | Hs.692 | GA733-2 antigen; epithelial glycoprotein | 3.1 | 353 | 116 | 2.8 |
| 101888 | AL049610 | Hs.95243 | transcription elongation factor A (SII)- | 7.3 | 73 | 1 | 5.3 |
| 101960 | AL036287 | Hs.194662 | calponin 3, acidic | 3.8 | 399 | 105 | 3.3 |
| 102009 | BE245149 | Hs.82643 | protein tyrosine kinase 9 | 4.6 | 148 | 32 | 11.3 |
| 102095 | U11313 | Hs.75760 | sterol carrier protein 2 | 9.5 | 95 | 4 | 8.8 |
| 102123 | NM_001809 | Hs.1594 | centromere protein A (17 kD) | 4.2 | 42 | 7 | 3.4 |
| 102125 | NM_006456 | Hs.288215 | sialyltransferase | 9.3 | 93 | 4 | 3 |
| 102139 | NM_004419 | Hs.2128 | dual specificity phosphatase 5 | 5.4 | 137 | 26 | 2.5 |
| 102162 | AA450274 | Hs.1592 | CDC16 (cell division cycle 16, S. cerevi | 4.6 | 151 | 33 | 2 |
| 102165 | BE313280 | Hs.159627 | death associated protein 3 | 9.3 | 93 | 5 | 8 |
| 102193 | AL036335 | Hs.313 | secreted phosphoprotein 1 (osteopontin, | 45.7 | 457 | 1 | 39.7 |
| 102211 | BE314524 | Hs.78776 | putative transmembrane protein | 3.9 | 442 | 114 | 1.3 |
| 102221 | NM_006769 | Hs.3844 | LIM domain only 4 | 4.9 | 49 | 1 | 3.6 |
| 102242 | U27185 | Hs.82547 | retinoic acid receptor responder (tazaro | 3.1 | 31 | 1 | 1.3 |
| 102258 | NM_001546 | Hs.34853 | inhibitor of DNA binding 4, dominant neg | 3.8 | 163 | 43 | 0.5 |
| 102302 | AA306342 | Hs.69171 | protein kinase C-like 2 | 4.5 | 45 | 1 | 3.6 |
| 102304 | AF015224 | Hs.46452 | mammaglobin 1 | 8.5 | 2058 | 243 | 1.4 |
| 102348 | U37519 | Hs.87539 | aldehyde dehydrogenase 3 family, member | 6.4 | 428 | 67 | 2.3 |
| 102369 | U39840 | Hs.299867 | hepatocyte nuclear factor 3, alpha | 6.7 | 67 | 9 | 6.3 |
| 102407 | AW602154 | Hs.82143 | E74-like factor 2 (ets domain transcript | 5.3 | 53 | 1 | 4.8 |
| 102409 | BE300330 | Hs.118725 | selenophosphate synthetase 2 | 3.3 | 111 | 34 | 7.5 |
| 102457 | NM_001394 | Hs.2359 | dual specificity phosphatase 4 | 20.2 | 202 | 5 | 1.3 |
| 102544 | NM_003937 | Hs.169139 | kynureninase (L-kynurenine hydrolase) | 3.8 | 38 | 1 | 1.5 |
| 102567 | U63830 | Hs.146847 | TRAF family member-associated NFKB activ | 8.2 | 82 | 1 | 6.8 |
| 102580 | U60808 | Hs.152981 | CDP-diacylglycerol synthase (phosphatida | 4.1 | 41 | 1 | 3.3 |
| 102618 | AL037672 | Hs.81071 | extracellular matrix protein 1 | 10.2 | 628 | 62 | 17.2 |
| 102638 | U67319 | Hs.9216 | caspase 7, apoptosis-related cysteine pr | 5 | 66 | 13 | 5.3 |
| 102663 | NM_002270 | Hs.168075 | karyopherin (importin) beta 2 | 6.1 | 126 | 21 | 2.4 |
| 102669 | U71207 | Hs.29279 | eyes absent (Drosophila) homolog 2 | 4.5 | 45 | 1 | 2.8 |
| 102742 | U79293 | Hs.159264 | Human clone 23948 mRNA sequence | 4.1 | 41 | 1 | 2.4 |
| 102784 | U85658 | Hs.61796 | transcription factor AP-2 gamma (activat | 4.4 | 255 | 58 | 1.6 |
| 102805 | U90304 | Hs.25351 | iroquois homeobox protein 5 | 3.6 | 142 | 39 | 1.6 |
| 102813 | BE242035 | Hs.151461 | embryonic ectoderm development | 3.5 | 35 | 1 | 2.7 |
| 102823 | D85390 | Hs.5057 | carboxypeptidase D | 5.6 | 56 | 1 | 5.3 |
| 102825 | BE262386 | Hs.7137 | clones 23667 and 23775 zinc finger prote | 4.2 | 42 | 7 | 3.7 |
| 102899 | AI815559 | Hs.75730 | signal recognition particle receptor (‘d | 3.2 | 58 | 18 | 5 |
| 102913 | NM_002275 | Hs.80342 | keratin 15 | 5.8 | 753 | 131 | 0.4 |
| 102927 | BE512730 | Hs.65114 | keratin 18 | 3.1 | 815 | 266 | 1.7 |
| 102961 | AL119505 | Hs.198166 | activating transcription factor 2 | 3.2 | 32 | 4 | 2.6 |
| 102968 | AU076611 | Hs.154672 | methylene tetrahydrofolate dehydrogenase | 5.7 | 251 | 44 | 6.6 |
| 103003 | AI910275 | Hs.1406 | trefoil factor 1 (pS2) | 5.6 | 1346 | 239 | 5.4 |
| 103023 | AW500470 | Hs.117950 | multifunctional polypeptide similar to S | 5.8 | 218 | 38 | 13 |
| 103024 | NM_002343 | Hs.105938 | lactotransferrin | 3.7 | 1421 | 388 | 1.9 |
| 103036 | M13509 | Hs.83169 | matrix metalloproteinase 1 (MMP1; inters | 3.1 | 94 | 30 | 5.8 |
| 103038 | AA926960 | Hs.334883 | CDC28 protein kinase 1 | 3.5 | 332 | 94 | 3.1 |
| 103119 | X63629 | Hs.2877 | cadherin 3, type 1, P-cadherin (placenta | 4.8 | 312 | 65 | 30.9 |
| 103134 | X65724 | Hs.2839 | Norrie disease (pseudoglioma) | 5.2 | 331 | 64 | 1.5 |
| 103134 | X65724 | Hs.2839 | Norrie disease (pseudoglioma) | 4.9 | 49 | 5 | 3.8 |
| 103171 | AW583058 | Hs.234726 | serine (or cysteine) proteinase inhibito | 3.3 | 1497 | 458 | 2.1 |
| 103206 | X72755 | Hs.77367 | monokine induced by gamma interferon | 3.5 | 796 | 228 | 3.2 |
| 103208 | AW411340 | Hs.31314 | retinoblastoma-binding protein 7 | 5.6 | 191 | 34 | 3.5 |
| 103226 | X75042 | Hs.44313 | v-rel avian reticuloendotheliosis viral | 4.1 | 53 | 13 | 4.9 |
| 103333 | AA206186 | Hs.79889 | monocyte to macrophage differentiation-a | 3.4 | 34 | 8 | 2.3 |
| 103346 | X87613 | Hs.5464 | thyroid hormone receptor coactivating pr | 3.9 | 43 | 11 | 1 |
| 103352 | H09366 | Hs.78853 | uracil-DNA glycosylase | 9.3 | 93 | 8 | 8.2 |
| 103375 | NM_005982 | Hs.54416 | sine oculis homeobox (Drosophila) homolo | 9.7 | 97 | 1 | 9.3 |
| 103376 | AL036166 | Hs.323378 | coated vesicle membrane protein | 6.3 | 98 | 16 | 9.1 |
| 103391 | X94453 | Hs.114366 | pyrroline-5-carboxylate synthetase (glut | 4.3 | 77 | 18 | 7.2 |
| 103438 | AW175781 | Hs.152720 | M-phase phosphoprotein 6 | 4.9 | 153 | 31 | 2.4 |
| 103453 | AI878922 | Hs.180139 | SMT3 (suppressor of mif two 3, yeast) ho | 4.9 | 261 | 53 | 3.7 |
| 103471 | Y00815 | Hs.75216 | protein tyrosine phosphatase, receptor t | 3.5 | 564 | 162 | 1.7 |
| 103500 | AW408009 | Hs.22580 | alkylglycerone phosphate synthase | 3.9 | 49 | 13 | 2.5 |
| 103557 | AL133415 | Hs.297753 | vimentin | 7.5 | 136 | 18 | 3.4 |
| 103587 | BE270266 | Hs.82128 | 5T4 oncofetal trophoblast glycoprotein | 7.9 | 79 | 2 | 6.9 |
| 103605 | BE409838 | Hs.194657 | cadherin 1, type 1, E-cadherin (epitheli | 3.3 | 745 | 229 | 1.8 |
| 103606 | AW403814 | Hs.41714 | BCL2-associated athanogene | 3.2 | 41 | 13 | 2.8 |
| 103613 | NM_000346 | Hs.2316 | SRY (sex determining region Y)-box 9 (ca | 7.3 | 73 | 1 | 5.2 |
| 103658 | NM_000088 | Hs.172928 | collagen, type I, alpha 1 | 3.8 | 1612 | 429 | 3.1 |
| 103666 | NM_003528 | Hs.2178 | H2B histone family, member Q | 3.2 | 32 | 5 | 2.8 |
| 103988 | AA314389 | Hs.42500 | ADP-ribosylation factor-like 5 | 3.2 | 32 | 9 | 2.7 |
| 103990 | AB033112 | Hs.42179 | bromodomain and PHD finger containing, 3 | 4.9 | 49 | 1 | 4.2 |
| 104052 | NM_002407 | Hs.97644 | mammaglobin 2 | 7.2 | 498 | 69 | 9.3 |
| 104115 | AF183810 | Hs.26102 | opposite strand to trichorhinophalangeal | 29 | 290 | 1 | 26.8 |
| 104129 | H63349 | Hs.98806 | hypothetical protein | 3.7 | 37 | 7 | 2.1 |
| 104146 | AW880614 | Hs.146381 | RNA binding motif protein, X chromosome | 5.2 | 52 | 1 | 4.3 |
| 104147 | BE081342 | Hs.283037 | HSPC039 protein | 8 | 84 | 11 | 6.3 |
| 104209 | AB012113 | Hs.16530 | small inducible cytokine subfamily A (Cy | 5.8 | 58 | 1 | 3.2 |
| 104239 | AB002367 | Hs.21355 | doublecortin and CaM kinase-like 1 | 6.4 | 64 | 8 | 3 |
| 104278 | AW583693 | Hs.109253 | N-terminal acetyltransferase complex ard | 4.7 | 229 | 49 | 7.9 |
| 104309 | AI337300 | Hs.284123 | hypothetical protein MGC4604 | 3.2 | 32 | 7 | 2.4 |
| 104394 | AA129551 | Hs.172129 | Homo sapiens cDNA: FLJ21409 fis, clone C | 5.3 | 144 | 27 | 13.1 |
| 104432 | X51501 | Hs.99949 | prolactin-induced protein | 6.9 | 1494 | 218 | 1.3 |
| 104558 | R56678 | Hs.88959 | hypothetical protein MGC4816 | 7.7 | 77 | 8 | 6.9 |
| 104567 | AA040620 | Hs.5672 | hypothetical protein AF140225 | 3.7 | 37 | 5 | 2.5 |
| 104590 | AW373062 | Hs.83623 | nuclear receptor subfamily 1, group I, m | 6.1 | 493 | 81 | 0.7 |
| 104602 | H47610 | | gb: yp75f03.s1 Soares fetal liver spleen | 3.8 | 38 | 4 | 1.2 |
| 104613 | AF123303 | Hs.24713 | hypothetical protein | 4.8 | 231 | 49 | 7.3 |
| 104633 | H00820 | Hs.30977 | ESTs, Weakly similar to B34087 hypotheti | 3.4 | 154 | 46 | 3 |
| 104636 | R82252 | Hs.106106 | protein kinase (cAMP-dependent, catalyti | 5 | 468 | 94 | 4.7 |
| 104660 | BE298665 | Hs.14846 | Homo sapiens mRNA; cDNA DKFZp564D016 (fr | 3.8 | 82 | 22 | 3.1 |
| 104667 | AI239923 | Hs.30098 | ESTs | 14.9 | 149 | 1 | 6.4 |
| 104766 | BE244072 | Hs.20815 | macrophage erythroblast attacher | 6.3 | 165 | 26 | 3.2 |
| 104787 | AA027317 | | gb: ze97d11.s1 Soares_fetal_heart_NbHH19W | 3.8 | 40 | 11 | 3.8 |
| 104804 | AI858702 | Hs.31803 | ESTs, Weakly similar to N-WASP [H. sapien | 7.7 | 77 | 1 | 5.1 |
| 104807 | AI139058 | Hs.125790 | leucine-rich repeat-containing 2 | 7 | 70 | 1 | 6.5 |
| 104846 | AI250789 | Hs.32478 | ESTs | 4.7 | 201 | 43 | 4.5 |
| 104896 | AW015318 | Hs.23165 | ESTs | 7.4 | 74 | 1 | 6 |
| 104919 | AA026880 | Hs.25252 | prolactin receptor | 3.9 | 280 | 72 | 3.3 |
| 104926 | BE298808 | Hs.33363 | DKFZP434N093 protein | 4.2 | 135 | 32 | 4 |
| 104943 | AF072873 | Hs.114218 | frizzled (Drosophila) homolog 6 | 16.2 | 162 | 1 | 4.2 |
| 104968 | AI249502 | Hs.29669 | ESTs | 3.8 | 38 | 1 | 2.4 |
| 104977 | AI392640 | Hs.18272 | amino acid transporter system A1 | 3.2 | 522 | 165 | 1.9 |
| 104997 | AA121686 | Hs.10592 | ESTs | 3.2 | 32 | 4 | 2.9 |
| 105029 | AI122691 | Hs.13268 | ESTs | 3.7 | 157 | 43 | 3.6 |
| 105038 | AW503733 | Hs.9414 | KIAA1488 protein | 5.5 | 55 | 1 | 5.2 |
| 105041 | AB037716 | Hs.26204 | KIAA1295 protein | 10.3 | 103 | 1 | 3.9 |
| 105086 | AA148710 | Hs.79914 | lumican | 6.6 | 66 | 1 | 5.4 |
| 105088 | H58589 | Hs.35156 | Homo sapiens cDNA FLJ11027 fis, clone PL | 3.1 | 31 | 1 | 2.5 |
| 105091 | AA148859 | Hs.179909 | hypothetical protein FLJ22995 | 3.2 | 32 | 1 | 3 |
| 105143 | AI368836 | Hs.24808 | ESTs, Weakly similar to I38022 hypotheti | 7.3 | 73 | 1 | 3.8 |
| 105154 | AA307279 | Hs.35947 | methyl-CpG binding domain protein 4 | 4.2 | 90 | 22 | 2.8 |
| 105162 | AL133033 | Hs.4084 | KIAA1025 protein | 6 | 60 | 6 | 4.6 |
| 105167 | AW612147 | Hs.32058 | Homo sapiens C1orf19 mRNA, partial cds | 3.8 | 38 | 2 | 3.2 |
| 105178 | AA313825 | Hs.21941 | AD036 protein | 9.3 | 436 | 47 | 5.8 |
| 105195 | AA975096 | Hs.19522 | hypothetical protein PRO2849 | 5.7 | 57 | 8 | 5.3 |
| 105200 | AA328102 | Hs.24641 | cytoskeleton associated protein 2 | 4.5 | 45 | 1 | 3.6 |
| 105248 | AW952479 | Hs.22826 | tropomodulin 3 (ubiquitous) | 4.3 | 43 | 1 | 3.9 |
| 105252 | AB039670 | Hs.9728 | ALEX1 protein | 8 | 80 | 6 | 7.3 |
| 105253 | AW997484 | Hs.5003 | KIAA0456 protein | 3.9 | 39 | 6 | 3.2 |
| 105280 | AA894638 | Hs.14600 | ESTs | 3.5 | 35 | 7 | 2.7 |
| 105288 | N99673 | Hs.3585 | ESTs, Weakly similar to AF126743 1 DNAJ | 4.5 | 45 | 10 | 0.5 |
| 105309 | AK000796 | Hs.4104 | hypothetical protein | 3.8 | 93 | 25 | 7.5 |
| 105329 | AA234561 | Hs.22862 | ESTs | 2.8 | 131 | 47 | 3.9 |
| 105344 | AF151073 | Hs.8645 | hypothetical protein | 3.9 | 79 | 20 | 6.5 |
| 105376 | AW994032 | Hs.8768 | hypothetical protein FLJ10849 | 5.1 | 181 | 36 | 15.8 |
| 105386 | AW500718 | Hs.8115 | Homo sapiens, clone MGC: 16169, mRNA, com | 4.1 | 41 | 2 | 3.3 |
| 105400 | AF198620 | Hs.65648 | RNA binding motif protein 8A | 6.2 | 62 | 6 | 5.6 |
| 105426 | W20027 | Hs.23439 | ESTs | 3.3 | 206 | 63 | 2.2 |
| 105483 | AL137257 | Hs.23458 | Homo sapiens cDNA: FLJ23015 fis, clone L | 3.2 | 466 | 146 | 8.4 |
| 105496 | AL117441 | Hs.301997 | hypothetical protein FLJ13033 | 16.6 | 166 | 8 | 12.7 |
| 105500 | AW602166 | Hs.222399 | CEGP1 protein | 25.4 | 508 | 20 | 3 |
| 105508 | AA173942 | Hs.326416 | Homo sapiens mRNA; cDNA DKFZp564H1916 (f | 9 | 117 | 13 | 10.6 |
| 105511 | AB037829 | Hs.3862 | regulator of nonsense transcripts 2; DKF | 3.2 | 32 | 6 | 1.5 |
| 105516 | AK001269 | Hs.30738 | hypothetical protein FLJ10407 | 8.3 | 83 | 3 | 1.8 |
| 105539 | AB040884 | Hs.109694 | KIAA1451 protein | 3.5 | 73 | 21 | 1.6 |
| 105564 | BE616694 | Hs.288042 | hypothetical protein FLJ14299 | 5.8 | 336 | 58 | 2 |
| 105610 | AA280072 | Hs.99872 | fetal Alzheimer antigen | 3.2 | 32 | 1 | 1 |
| 105616 | R35343 | Hs.24968 | Human DNA sequence from clone RP1-233G16 | 4.8 | 79 | 17 | 5.2 |
| 105627 | AA281279 | Hs.23317 | hypothetical protein FLJ14681 | 4 | 75 | 19 | 1.7 |
| 105640 | AA001021 | Hs.6685 | thyroid hormone receptor interactor 8 | 4.5 | 45 | 1 | 3.7 |
| 105645 | AW294631 | Hs.11325 | ESTs | 3.6 | 36 | 1 | 0.1 |
| 105674 | AI609530 | Hs.279789 | histone deacetylase 3 | 6.4 | 64 | 8 | 6 |
| 105687 | NM_014517 | Hs.28423 | upstream binding protein 1 (LBP-1a) | 4.7 | 152 | 33 | 5.3 |
| 105691 | AI680737 | Hs.289068 | Homo sapiens cDNA FLJ11918 fis, clone HE | 5.7 | 57 | 8 | 4.1 |
| 105730 | AW377314 | Hs.5364 | DKFZP564I052 protein | 6.9 | 69 | 1 | 4.4 |
| 105731 | AA834664 | Hs.29131 | nuclear receptor coactivator 2 | 3.4 | 34 | 1 | 3.1 |
| 105743 | BE246502 | Hs.9598 | sema domain, immunoglobulin domain (Ig), | 3 | 30 | 10 | 0.9 |
| 105759 | AI123118 | Hs.15159 | chemokine-like factor, alternatively spl | 5.4 | 54 | 1 | 4.4 |
| 105772 | H57111 | Hs.221132 | ESTs | 5.3 | 67 | 13 | 5.3 |
| 105774 | AW369278 | Hs.23412 | hypothetical protein FLJ20160 | 4.9 | 49 | 1 | 4.5 |
| 105784 | W84446 | Hs.226434 | hypothetical protein MGC4643 | 3.3 | 98 | 30 | 4.7 |
| 105795 | AA878183 | Hs.17448 | Homo sapiens cDNA FLJ13618 fis, clone PL | 3.2 | 143 | 46 | 3.6 |
| 105806 | AF206019 | Hs.110347 | REV1 (yeast homolog)-like | 4 | 40 | 3 | 3.2 |
| 105807 | AA788946 | Hs.16869 | ESTs, Moderately similar to CA1C RAT COL | 4.7 | 747 | 158 | 5.7 |
| 105823 | AI559444 | Hs.293960 | ESTs | 3.9 | 371 | 94 | 4.6 |
| 105832 | AW802282 | Hs.22265 | pyruvate dehydrogenase phosphatase | 3.6 | 68 | 19 | 6 |
| 105840 | AA601518 | Hs.22209 | secreted modular calcium-binding protein | 4.8 | 134 | 28 | 3.2 |
| 105851 | AI827976 | Hs.24391 | hypothetical protein FLJ13612 | 4.3 | 772 | 179 | 1.7 |
| 105864 | AI640775 | Hs.28332 | Homo sapiens cDNA: FLJ21560 fis, clone C | 4.3 | 43 | 1 | 3.7 |
| 105870 | AW021691 | Hs.101067 | GCN5 (general control of amino-acid synt | 3.6 | 36 | 7 | 3.1 |
| 105875 | AK001708 | Hs.32271 | hypothetical protein FLJ10846 | 3.4 | 34 | 8 | 2.9 |
| 105886 | AK001735 | Hs.22983 | UDP-glucose: glycoprotein glucosyltransfe | 3.6 | 45 | 13 | 1.3 |
| 105906 | N25986 | Hs.22380 | ESTs | 3.4 | 34 | 1 | 1.5 |
| 106012 | AI240665 | Hs.8895 | ESTs | 21.2 | 212 | 6 | 17.4 |
| 106020 | AA043039 | Hs.7870 | hypothetical protein | 3.9 | 47 | 12 | 4.4 |
| 106024 | AL122072 | Hs.103804 | heterogeneous nuclear ribonucleoprotein | 4.4 | 174 | 40 | 1.6 |
| 106034 | AW952005 | Hs.14928 | hypothetical protein FLJ12903 | 4.7 | 47 | 1 | 4 |
| 106036 | AA382267 | Hs.10653 | ESTs | 3.4 | 49 | 15 | 4.4 |
| 106055 | AA417034 | | gb: zu04f10.s1 Soares_testis_NHT Homo sap | 3.5 | 53 | 15 | 1.2 |
| 106057 | BE614474 | Hs.289074 | F-box only protein 22 | 3.4 | 116 | 35 | 2.2 |
| 106060 | NM_001329 | Hs.171391 | C-terminal binding protein 2 | 3.6 | 444 | 125 | 4.6 |
| 106070 | T74445 | Hs.5957 | Homo sapiens clone 24416 mRNA sequence | 3.6 | 365 | 103 | 6.9 |
| 106095 | AF115402 | Hs.11713 | E74-like factor 5 (ets domain transcript | 26.3 | 356 | 14 | 1 |
| 106096 | AW379378 | Hs.170121 | protein tyrosine phosphatase, receptor t | 3.2 | 267 | 83 | 2.3 |
| 106126 | AA576953 | Hs.22972 | hypothetical protein FLJ13352 | 3.8 | 38 | 1 | 3.3 |
| 106155 | AA425414 | Hs.33287 | nuclear factor I/B | 9.9 | 483 | 49 | 1.8 |
| 106157 | W37943 | Hs.34892 | KIAA1323 protein | 6.7 | 94 | 14 | 8 |
| 106198 | AI244563 | Hs.325531 | Homo sapiens clone 015h12 My015 protein | 3.3 | 95 | 29 | 4.4 |
| 106236 | AB040896 | Hs.21104 | KIAA1463 protein | 3.8 | 83 | 22 | 7.5 |
| 106286 | AI765107 | Hs.274422 | hypothetical protein FLJ20550 | 3.3 | 97 | 30 | 6.4 |
| 106290 | AW961393 | Hs.16364 | hypothetical protein FLJ10955 | 4.5 | 116 | 26 | 4.5 |
| 106310 | R98185 | Hs.17240 | ESTs | 7 | 70 | 3 | 1.3 |
| 106323 | AB007866 | Hs.158249 | KIAA0406 gene product | 3.2 | 37 | 12 | 2.6 |
| 106330 | AW977397 | Hs.35580 | ESTs | 3.8 | 38 | 1 | 1.9 |
| 106383 | AA447453 | Hs.27860 | Homo sapiens mRNA; cDNA DKFZp586M0723 (f | 16 | 255 | 16 | 6.6 |
| 106389 | AW748420 | Hs.6236 | Homo sapiens cDNA: FLJ21487 fis, clone C | 4.9 | 337 | 70 | 2.7 |
| 106394 | Z42993 | Hs.25320 | Homo sapiens clone 25142 mRNA sequence | 3.1 | 72 | 23 | 5 |
| 106432 | AK000310 | Hs.17138 | hypothetical protein FLJ20303 | 3.1 | 165 | 54 | 1.6 |
| 106459 | AA789081 | Hs.4029 | glioma-amplified sequence-41 | 3.1 | 31 | 1 | 2.6 |
| 106503 | AB033042 | Hs.29679 | cofactor required for Sp1 transcriptiona | 5.5 | 147 | 27 | 4.4 |
| 106508 | AI205785 | Hs.30348 | ESTs | 4.4 | 222 | 51 | 1.8 |
| 106565 | NM_014892 | Hs.227602 | KIAA1116 protein | 7.4 | 74 | 3 | 1.7 |
| 106586 | AA243837 | Hs.57787 | ESTs | 15.2 | 152 | 1 | 12.6 |
| 106589 | AK000933 | Hs.28661 | Homo sapiens cDNA FLJ10071 fis, clone HE | 3.8 | 263 | 69 | 3.9 |
| 106596 | AA452379 | Hs.293552 | ESTs, Moderately similar to ALU7_HUMAN A | 4.9 | 49 | 1 | 4.1 |
| 106611 | R49131 | Hs.26267 | ATP-dependant interferon response protei | 5.8 | 58 | 5 | 3.1 |
| 106628 | AW188205 | Hs.12311 | Homo sapiens clone 23570 mRNA sequence | 5.3 | 166 | 32 | 14.9 |
| 106650 | AL049951 | Hs.22370 | Homo sapiens mRNA; cDNA DKFZp564O0122 (f | 5.4 | 75 | 14 | 0.8 |
| 106683 | BE296396 | Hs.14512 | DIPB protein | 3.6 | 210 | 58 | 4.7 |
| 106698 | N28524 | Hs.29403 | hypothetical protein FLJ22060 | 5.7 | 57 | 10 | 4.8 |
| 106710 | N38902 | Hs.334437 | hypothetical protein MGC4248 | 4.4 | 371 | 84 | 3.2 |
| 106717 | AA600357 | Hs.239489 | TIA1 cytotoxic granule-associated RNA-bi | 4.3 | 101 | 24 | 1.6 |
| 106747 | NM_007118 | Hs.171957 | triple functional domain (PTPRF interact | 4.6 | 46 | 1 | 4 |
| 106834 | AL044182 | Hs.28070 | KIAA0753 gene product | 3.5 | 58 | 17 | 1.6 |
| 106846 | AB037744 | Hs.34892 | KIAA1323 protein | 5.4 | 192 | 36 | 4.4 |
| 106868 | BE185536 | Hs.301183 | molecule possessing ankyrin repeats indu | 3.3 | 696 | 214 | 1.8 |
| 106882 | AA149537 | Hs.26994 | hypothetical protein FLJ20477 | 3.8 | 38 | 1 | 1.6 |
| 106893 | AA835868 | Hs.25253 | mannosidase, alpha, class 1A, member 1 | 4.3 | 43 | 10 | 2.2 |
| 106895 | AK001826 | Hs.25245 | hypothetical protein FLJ11269 | 3.6 | 36 | 1 | 1.2 |
| 106897 | AF039023 | Hs.167496 | RAN binding protein 6 | 4.5 | 45 | 1 | 3.8 |
| 106916 | AA134329 | Hs.24170 | Homo sapiens, clone IMAGE: 3685398, mRNA, | 5.7 | 94 | 17 | 7.3 |
| 106962 | AI868648 | Hs.22315 | ESTs | 3.5 | 180 | 52 | 2.3 |
| 106968 | AF216751 | Hs.26813 | CDA14 | 5.5 | 130 | 24 | 12.5 |
| 106990 | AA280722 | Hs.24758 | ESTs, Weakly similar to I38022 hypotheti | 3.2 | 266 | 83 | 1.8 |
| 107008 | AL157479 | Hs.23740 | KIAA1598 protein | 5.1 | 298 | 59 | 4.4 |
| 107014 | AA598820 | | gb: ae36h12.s1 Gessler Wilms tumor Homo s | 3.3 | 228 | 69 | 2.8 |
| 107032 | AV650537 | Hs.247309 | succinate-CoA ligase, GDP-forming, beta | 3.1 | 55 | 18 | 3.8 |
| 107056 | AW401864 | Hs.18720 | programmed cell death 8 (apoptosis-induc | 3.1 | 75 | 24 | 2.2 |
| 107071 | AW385224 | Hs.35198 | ectonucleotide pyrophosphatase/phosphodi | 3.1 | 367 | 119 | 2.3 |
| 107080 | AL122043 | Hs.19221 | hypothetical protein DKFZp566G1424 | 3.9 | 98 | 25 | 8.6 |
| 107102 | AB037765 | Hs.30652 | KIAA1344 protein | 6.3 | 63 | 1 | 5.4 |
| 107109 | AA249096 | Hs.32793 | ESTs | 4.6 | 71 | 16 | 3.6 |
| 107136 | AV661958 | Hs.8207 | GK001 protein | 2.5 | 392 | 155 | 4.3 |
| 107151 | AW378065 | Hs.8687 | ESTs | 15.6 | 156 | 7 | 10.8 |
| 107217 | AL080235 | Hs.35861 | DKFZP586E1621 protein | 4.8 | 48 | 8 | 3.1 |
| 107222 | BE172058 | Hs.82689 | tumor rejection antigen (gp96) 1 | 3.4 | 251 | 74 | 23.7 |
| 107240 | AI290284 | Hs.159872 | ESTs | 3.6 | 36 | 6 | 0.5 |
| 107248 | AW263124 | Hs.315111 | nuclear receptor co-repressor/HDAC3 comp | 5.4 | 483 | 90 | 4 |
| 107295 | AA186629 | Hs.80120 | UDP-N-acetyl-alpha-D-galactosamine: polyp | 4.6 | 199 | 44 | 19.2 |
| 107299 | BE277457 | Hs.30661 | hypothetical protein MGC4606 | 12.5 | 156 | 13 | 2.9 |
| 107316 | T63174 | Hs.193700 | Homo sapiens mRNA; cDNA DKFZp586I0324 (f | 3.2 | 110 | 35 | 9.6 |
| 107318 | T74445 | Hs.5957 | Homo sapiens clone 24416 mRNA sequence | 3.5 | 35 | 1 | 2.6 |
| 107485 | AL042613 | Hs.262476 | S-adenosylmethionine decarboxylase 1 | 5.8 | 151 | 26 | 11.4 |
| 107612 | AI498986 | Hs.60090 | Homo sapiens cDNA FLJ13595 fis, clone PL | 3.2 | 32 | 5 | 2.1 |
| 107638 | AI580492 | Hs.42743 | hypothetical protein | 4.4 | 73 | 17 | 6.2 |
| 107727 | AA149707 | Hs.173091 | ubiquitin-like 3 | 3.5 | 282 | 80 | 3.7 |
| 107859 | AW732573 | Hs.47584 | potassium voltage-gated channel, delayed | 5.7 | 85 | 15 | 7.8 |
| 107876 | AW372451 | Hs.61184 | CGI-79 protein | 3.5 | 35 | 1 | 1 |
| 107884 | AA054949 | Hs.61307 | ESTs | 4.3 | 43 | 10 | 2.7 |
| 107886 | AA025782 | Hs.61284 | ESTs | 3.1 | 31 | 9 | 2.2 |
| 107908 | AF087999 | Hs.42826 | ESTs | 4.7 | 47 | 4 | 4.3 |
| 107922 | BE153855 | Hs.61460 | Ig superfamily receptor LNIR | 9 | 90 | 1 | 5.5 |
| 107994 | AA036811 | Hs.48469 | LIM domains containing 1 | 4.5 | 45 | 1 | 3.8 |
| 108040 | AL121031 | Hs.159971 | SWI/SNF related, matrix associated, acti | 6.5 | 65 | 2 | 6 |
| 108055 | AJ404672 | Hs.334483 | hypothetical protein FLJ23571 | 7.4 | 74 | 8 | 6 |
| 108063 | BE548479 | Hs.14838 | hypothetical protein FLJ10773 | 3.4 | 34 | 1 | 2.3 |
| 108339 | AW151340 | Hs.51615 | ESTs, Weakly similar to ALU7_HUMAN ALU S | 18.7 | 187 | 1 | 17 |
| 108467 | AI478658 | Hs.94631 | brefeldin A-inhibited guanine nucleotide | 3.8 | 38 | 1 | 3.2 |
| 108539 | AA084677 | Hs.54558 | hypothetical protein FLJ22222 | 5.7 | 57 | 1 | 4.9 |
| 108634 | AW022410 | Hs.69507 | ESTs | 3.2 | 32 | 5 | 1.7 |
| 108647 | BE546947 | Hs.44276 | homeo box C10 | 8.7 | 247 | 29 | 5.7 |
| 108695 | AB029000 | Hs.70823 | KIAA1077 protein | 3.7 | 625 | 168 | 3.8 |
| 108778 | AF133123 | Hs.90847 | general transcription factor IIIC, polyp | 3.7 | 37 | 1 | 3.2 |
| 108806 | AF070578 | Hs.71168 | Homo sapiens clone 24674 mRNA sequence | 3.4 | 34 | 1 | 2.8 |
| 108807 | AI652236 | Hs.49376 | hypothetical protein FLJ20644 | 3.5 | 35 | 1 | 3.2 |
| 108810 | AW295647 | Hs.71331 | hypothetical protein MGC5350 | 5.3 | 53 | 1 | 2.8 |
| 108846 | AL117452 | Hs.44155 | DKFZP586G1517 protein | 4.8 | 96 | 20 | 6.5 |
| 108857 | AK001468 | Hs.62180 | anillin (Drosophila Scraps homolog), act | 5.4 | 54 | 1 | 4 |
| 108893 | BE276891 | Hs.194691 | retinoic acid induced 3 | 3.1 | 529 | 170 | 4.1 |
| 108917 | AI380268 | Hs.173648 | ESTs, Weakly similar to Zinc-finger prot | 3.3 | 33 | 5 | 1.7 |
| 109010 | NM_007240 | Hs.44229 | dual specificity phosphatase 12 | 3.4 | 34 | 1 | 2.6 |
| 109060 | BE062109 | Hs.241551 | chloride channel, calcium activated, fam | 3.1 | 31 | 8 | 2 |
| 109101 | AW608930 | Hs.52184 | hypothetical protein FLJ20618 | 3.4 | 71 | 21 | 2.4 |
| 109112 | AW419196 | Hs.257924 | hypothetical protein FLJ13782 | 4.1 | 334 | 82 | 3.4 |
| 109124 | AK000684 | Hs.183887 | hypothetical protein FLJ22104 | 3.3 | 33 | 1 | 2.9 |
| 109128 | H89083 | Hs.181915 | ESTs | 4 | 40 | 7 | 1.1 |
| 109160 | BE220601 | Hs.301997 | hypothetical protein FLJ13033 | 3.8 | 233 | 62 | 3.8 |
| 109166 | AA219691 | Hs.73625 | RAB6 interacting, kinesin-like (rabkines | 8.8 | 199 | 23 | 16.1 |
| 109173 | AA179962 | Hs.73643 | EST | 3.2 | 32 | 1 | 2.2 |
| 109178 | AW976516 | Hs.283707 | Homo sapiens cDNA: FLJ21354 fis, clone C | 3.2 | 32 | 10 | 2.9 |
| 109235 | AI381800 | Hs.300684 | calcitonin gene-related peptide-receptor | 4.9 | 121 | 25 | 10.4 |
| 109273 | AA375752 | Hs.82719 | Homo sapiens mRNA; cDNA DKFZp586F1822 (f | 2.9 | 114 | 39 | 9.9 |
| 109292 | AW975746 | Hs.188662 | KIAA1702 protein | 7.1 | 71 | 1 | 6.5 |
| 109391 | AL096858 | Hs.184245 | KIAA0929 protein Msx2 interacting nuclea | 6.9 | 69 | 5 | 6.2 |
| 109411 | R98881 | Hs.109655 | sex comb on midleg (Drosophila)-like 1 | 3.3 | 39 | 12 | 1.5 |
| 109412 | BE543313 | Hs.209473 | hypothetical protein FLJ10520 | 4.2 | 56 | 14 | 2.2 |
| 109415 | U80736 | Hs.110826 | trinucleotide repeat containing 9 | 12.3 | 123 | 1 | 11.3 |
| 109481 | AA878923 | Hs.289069 | hypothetical protein FLJ21016 | 3.2 | 286 | 91 | 5.7 |
| 109517 | AI631874 | Hs.155140 | casein kinase 2, alpha 1 polypeptide | 8.3 | 83 | 8 | 1.9 |
| 109597 | AA989362 | Hs.293780 | ESTs | 5.9 | 59 | 10 | 4.2 |
| 109729 | F10024 | Hs.268740 | ESTs | 3.2 | 41 | 13 | 3.3 |
| 109795 | AA173942 | Hs.326416 | Homo sapiens mRNA; cDNA DKFZp564H1916 (f | 5.9 | 208 | 36 | 1.8 |
| 109799 | AW965076 | Hs.180378 | hypothetical protein 669 | 5 | 50 | 5 | 4.1 |
| 109883 | R68827 | Hs.95011 | syntrophin, beta 1 (dystrophin-associate | 3.7 | 37 | 4 | 2 |
| 109912 | AW390822 | Hs.301528 | L-kynurenine/alpha-aminoadipate aminotra | 14.2 | 142 | 1 | 9.5 |
| 109937 | AI084066 | Hs.20072 | myosin regulatory light chain interactin | 4.1 | 41 | 7 | 1.7 |
| 109958 | AA001266 | Hs.133521 | ESTs | 4.2 | 58 | 14 | 0.8 |
| 109984 | AI796320 | Hs.10299 | Homo sapiens cDNA FLJ13545 fis, clone PL | 3.2 | 136 | 43 | 3.6 |
| 110009 | BE075297 | Hs.6614 | ESTs, Weakly similar to A43932 mucin 2 p | 6.3 | 693 | 110 | 7.2 |
| 110240 | AI668594 | Hs.176588 | ESTs, Weakly similar to CP4Y_HUMAN CYTOC | 4.6 | 913 | 199 | 2.9 |
| 110369 | AK000768 | Hs.107872 | hypothetical protein FLJ20761 | 3.8 | 38 | 7 | 2.8 |
| 110426 | AI610702 | Hs.28212 | ESTs, Weakly similar to TRHY_HUMAN TRICH | 6.7 | 78 | 12 | 3 |
| 110478 | H11236 | Hs.31034 | peroxisomal biogenesis factor 11A | 3.7 | 37 | 1 | 2.1 |
| 110481 | AF075089 | Hs.36823 | ESTs | 3.6 | 36 | 10 | 2.5 |
| 110581 | H61560 | | gb: yr22g03.s1 Soares fetal liver spleen | 3.3 | 33 | 1 | 1.8 |
| 110674 | AA071276 | Hs.19469 | KIAA0859 protein | 3.5 | 35 | 8 | 1.9 |
| 110705 | AB007902 | Hs.32168 | KIAA0442 protein | 3.6 | 282 | 79 | 1.7 |
| 110721 | H97678 | Hs.31319 | ESTs | 4.4 | 103 | 24 | 3.8 |
| 110731 | NM_014899 | Hs.188006 | KIAA0878 protein | 3.3 | 138 | 42 | 3.6 |
| 110769 | BE000831 | Hs.23837 | Homo sapiens cDNA FLJ11812 fis, clone HE | 13.5 | 135 | 1 | 5.1 |
| 110775 | N22414 | | gb: yw39a07.s1 Weizmann Olfactory Epithel | 5.4 | 54 | 1 | 3.7 |
| 110787 | AA831267 | Hs.12244 | hypothetical protein FLJ20097 | 4.7 | 47 | 4 | 4.2 |
| 110799 | AI089660 | Hs.323401 | dpy-30-like protein | 5 | 50 | 1 | 4.3 |
| 110818 | AL157503 | Hs.27552 | Homo sapiens mRNA; cDNA DKFZp586N2424 (f | 3.1 | 31 | 1 | 2.7 |
| 110839 | AF153330 | Hs.30246 | solute carrier family 19 (thiamine trans | 8.4 | 84 | 1 | 5.3 |
| 110844 | AI740792 | Hs.167531 | methylcrotonoyl-Coenzyme A carboxylase 2 | 10.5 | 105 | 4 | 7.1 |
| 110854 | BE612992 | Hs.27931 | hypothetical protein FLJ10607 similar to | 7.9 | 79 | 1 | 6.2 |
| 110882 | AW963705 | Hs.301183 | molecule possessing ankyrin repeats indu | 3.9 | 353 | 90 | 1.2 |
| 110908 | AI433165 | Hs.9856 | ESTs | 3.1 | 31 | 1 | 1.3 |
| 110915 | BE092285 | Hs.29724 | hypothetical protein FLJ13187 | 20.9 | 209 | 1 | 19.5 |
| 110930 | BE242691 | Hs.14947 | ESTs | 3.4 | 115 | 34 | 2.4 |
| 110970 | Y19062 | Hs.96870 | staufen (Drosophila, RNA-binding protein | 3.5 | 35 | 9 | 3.2 |
| 111084 | H44186 | Hs.15456 | PDZ domain containing 1 | 4.3 | 43 | 1 | 2 |
| 111125 | N63823 | Hs.269115 | ESTs, Moderately similar to Z195_HUMAN Z | 5.4 | 54 | 1 | 4.3 |
| 111132 | AB037807 | Hs.83293 | hypothetical protein | 7.2 | 72 | 10 | 6.1 |
| 111164 | N46180 | Hs.122489 | Homo sapiens cDNA FLJ13289 fis, clone OV | 7.7 | 77 | 1 | 5 |
| 111179 | AK000136 | Hs.10760 | asporin (LRR class 1) | 25.1 | 288 | 12 | 6.7 |
| 111184 | AI815486 | Hs.243901 | Homo sapiens cDNA FLJ20738 fis, clone HE | 3.9 | 146 | 37 | 9.8 |
| 111190 | AK002055 | Hs.151046 | hypothetical protein FLJ11193 | 6.3 | 63 | 1 | 5.8 |
| 111221 | AB037782 | Hs.15119 | KIAA1361 protein | 3.7 | 119 | 33 | 6.7 |
| 111223 | AA852773 | Hs.334838 | KIAA1866 protein | 3.6 | 402 | 112 | 4.9 |
| 111229 | AW389845 | Hs.110855 | ESTs | 4.3 | 43 | 1 | 1 |
| 111234 | AA902656 | Hs.21943 | NIF3 (Ngg1 interacting factor 3, S.pombe | 3.3 | 33 | 1 | 1.1 |
| 111241 | AA345644 | Hs.288880 | PAN2 protein | 4.8 | 61 | 13 | 5.6 |
| 111345 | AW263155 | Hs.14559 | hypothetical protein FLJ10540 | 4.3 | 43 | 5 | 2.2 |
| 111353 | W20090 | Hs.6616 | ESTs | 4.1 | 41 | 1 | 2.6 |
| 111357 | BE314949 | Hs.87128 | hypothetical protein FLJ23309 | 3.8 | 425 | 111 | 4 |
| 111378 | AW160993 | Hs.326292 | hypothetical gene DKFZp434A1114 | 4.3 | 65 | 15 | 5.7 |
| 111389 | AK000987 | Hs.169111 | oxidation resistance 1 | 3.4 | 314 | 91 | 2.4 |
| 111540 | U82670 | Hs.9786 | zinc finger protein 275 | 3.5 | 35 | 1 | 2.1 |
| 111806 | BE071382 | Hs.279008 | hypothetical protein FLJ20170 | 3.5 | 105 | 30 | 9.6 |
| 111884 | AW502285 | Hs.127236 | hypothetical protein FLJ12879 | 3.2 | 37 | 12 | 3.5 |
| 111923 | BE383234 | Hs.25925 | Homo sapiens, clone MGC: 15393, mRNA, com | 6.2 | 62 | 2 | 5.9 |
| 111929 | AF027208 | Hs.112360 | prominin (mouse)-like 1 | 8.1 | 328 | 41 | 1.7 |
| 111942 | R40576 | Hs.21590 | hypothetical protein DKFZp564O0523 | 4.2 | 125 | 30 | 7.4 |
| 111987 | NM_015310 | Hs.6763 | KIAA0942 protein | 6.5 | 65 | 10 | 1.5 |
| 112092 | R44538 | | gb: yg29c02.s1 Soares infant brain 1NIB H | 3.3 | 33 | 10 | 2.3 |
| 112134 | R41823 | Hs.7413 | ESTs; calsyntenin-2 | 6.1 | 185 | 31 | 6.6 |
| 112197 | NM_003655 | Hs.5637 | ESTs | 3.5 | 507 | 145 | 3.3 |
| 112198 | AI432672 | Hs.288539 | hypothetical protein FLJ22191 | 3.5 | 40 | 12 | 2.5 |
| 112244 | AB029000 | Hs.70823 | KIAA1077 protein | 5.7 | 567 | 100 | 6.7 |
| 112253 | R51818 | | gb: yg77h12.s1 Soares infant brain 1NIB H | 4 | 70 | 18 | 6.8 |
| 112269 | R53734 | Hs.25978 | ESTs, Weakly similar to 2109260A B cell | 3.7 | 37 | 1 | 3 |
| 112275 | AW972635 | Hs.301904 | hypothetical protein FLJ12671 | 4.3 | 45 | 11 | 4.4 |
| 112280 | AA863360 | Hs.26040 | ESTs, Weakly similar to fatty acid omega | 2.8 | 751 | 270 | 1.3 |
| 112305 | AK000914 | Hs.26244 | hypothetical protein FLJ10052 | 3.5 | 41 | 12 | 3.7 |
| 112483 | AW969785 | Hs.285885 | Homo sapiens cDNA FLJ11321 fis, clone PL | 4.2 | 42 | 6 | 3.6 |
| 112513 | R68425 | Hs.13809 | hypothetical protein FLJ10648 | 4.7 | 54 | 12 | 4.5 |
| 112571 | AA412205 | Hs.140996 | ESTs | 4.8 | 48 | 2 | 3.4 |
| 112971 | Z42387 | Hs.83883 | transmembrane, prostate androgen induced | 4.5 | 390 | 87 | 5.3 |
| 113023 | AL134324 | Hs.7312 | ESTs | 3.2 | 99 | 31 | 3.1 |
| 113047 | AI571940 | Hs.7549 | ESTs | 9.6 | 124 | 13 | 9 |
| 113073 | N39342 | Hs.103042 | microtubule-associated protein 1B | 9.1 | 91 | 6 | 8.3 |
| 113083 | AA283057 | Hs.266957 | hypothetical protein FLJ14281 | 6.5 | 65 | 6 | 4.8 |
| 113287 | T66847 | Hs.194040 | ESTs, Weakly similar to I38022 hypotheti | 3.5 | 35 | 1 | 1.4 |
| 113296 | AW449560 | Hs.89576 | inner mitochondrial membrane peptidase 2 | 3.5 | 35 | 4 | 3.3 |
| 113523 | AI791905 | Hs.95549 | hypothetical protein | 7.6 | 76 | 1 | 4.2 |
| 113604 | AI075407 | Hs.296083 | ESTs, Moderately similar to I54374 gene | 3.1 | 453 | 148 | 7 |
| 113617 | AI869372 | Hs.17207 | Homo sapiens cDNA FLJ11922 fis, clone HE | 3.6 | 36 | 4 | 2.6 |
| 113702 | T97307 | | gb: ye53h05.s1 Soares fetal liver spleen | 12.3 | 129 | 11 | 11.7 |
| 113783 | AL359588 | Hs.7041 | hypothetical protein DKFZp762B226 | 4.6 | 46 | 4 | 4.3 |
| 113791 | AI269096 | Hs.135578 | chitobiase, di-N-acetyl- | 3.6 | 36 | 1 | 1.2 |
| 113794 | T62849 | Hs.11090 | membrane-spanning 4-domains, subfamily A | 3.3 | 744 | 227 | 2.5 |
| 113804 | BE247683 | Hs.14611 | dual specificity phosphatase 11 (RNA/RNP | 3.3 | 180 | 54 | 2.1 |
| 113808 | W44735 | Hs.9286 | Homo sapiens cDNA: FLJ21278 fis, clone C | 5.1 | 51 | 5 | 4.5 |
| 113847 | NM_005032 | Hs.4114 | plastin 3 (T isoform) | 3.2 | 238 | 75 | 2.1 |
| 113849 | AA457211 | Hs.8858 | bromodomain adjacent to zinc finger doma | 4.3 | 43 | 8 | 3.6 |
| 113867 | AW002834 | Hs.24095 | ESTs | 6.1 | 110 | 18 | 10.2 |
| 113886 | W76027 | Hs.23920 | hypothetical protein FLJ11105 | 4 | 48 | 12 | 4 |
| 113923 | AW953484 | Hs.3849 | hypothetical protein FLJ22041 similar to | 3.7 | 239 | 65 | 3.6 |
| 113936 | W17056 | Hs.83623 | nuclear receptor subfamily 1, group I, m | 4.3 | 819 | 191 | 1.2 |
| 113950 | AI267652 | Hs.30504 | Homo sapiens mRNA; cDNA DKFZp434E082 (fr | 10.7 | 123 | 12 | 7 |
| 114030 | AI825386 | Hs.164478 | hypothetical protein FLJ21939 similar to | 4.4 | 44 | 6 | 2.3 |
| 114051 | AB026436 | Hs.177534 | dual specificity phosphatase 10 | 4.5 | 45 | 4 | 2.6 |
| 114057 | AF116653 | Hs.34192 | Homo sapiens PRO0823 mRNA, complete cds | 3.5 | 35 | 6 | 3.2 |
| 114082 | AK001612 | Hs.26962 | Homo sapiens cDNA FLJ10750 fis, clone NT | 3.1 | 31 | 5 | 1.5 |
| 114124 | W57554 | Hs.125019 | lymphoid nuclear protein (LAF-4) mRNA | 24.2 | 242 | 10 | 5.6 |
| 114138 | AW384793 | Hs.15740 | Homo sapiens mRNA; cDNA DKFZp434E033 (fr | 6.7 | 67 | 1 | 6.3 |
| 114162 | AF155661 | Hs.22265 | pyruvate dehydrogenase phosphatase | 3.8 | 73 | 19 | 1.8 |
| 114196 | AF017445 | Hs.150926 | fucose-1-phosphate guanylyltransferase | 4.4 | 104 | 24 | 5.1 |
| 114208 | AL049466 | Hs.7859 | ESTs | 5.7 | 57 | 1 | 4.9 |
| 114239 | AL137667 | Hs.267445 | Homo sapiens mRNA; cDNA DKFZp434B231 (fr | 3.3 | 33 | 1 | 2.4 |
| 114251 | H15261 | Hs.21948 | ESTs | 4.2 | 46 | 11 | 1.4 |
| 114306 | AF100143 | Hs.6540 | fibroblast growth factor 13 | 4.5 | 45 | 2 | 3 |
| 114460 | AF183810 | Hs.26102 | trichorhinophalangeal syndrome I | 4.4 | 44 | 1 | 3 |
| 114542 | AW970128 | Hs.91011 | anterior gradient 2 (Xenepus laevis) hom | 4.7 | 770 | 166 | 5.8 |
| 114652 | AI521936 | Hs.107149 | novel protein similar to archaeal, yeast | 5.2 | 52 | 3 | 2.3 |
| 114767 | AI859865 | Hs.154443 | minichromosome maintenance deficient (S. | 4.6 | 196 | 43 | 10 |
| 114768 | AF212848 | Hs.182339 | ets homologous factor | 13.7 | 137 | 1 | 8.9 |
| 114774 | AV656017 | Hs.184325 | CGI-76 protein | 3.3 | 168 | 51 | 7.3 |
| 114798 | AA159181 | Hs.54900 | serologically defined colon cancer antig | 7.4 | 137 | 19 | 1.8 |
| 114821 | AI648602 | Hs.55468 | ESTs | 4.7 | 57 | 12 | 4.7 |
| 114860 | AL157545 | Hs.42179 | bromodomain and PHD finger containing, 3 | 9.1 | 91 | 1 | 7.6 |
| 114918 | BE165762 | Hs.23518 | hypothetical protein from BCRA2 region | 10.1 | 111 | 11 | 10.2 |
| 114940 | BE092696 | Hs.75928 | ESTs | 6.4 | 67 | 11 | 5 |
| 114965 | AI733881 | Hs.72472 | BMP-R1B | 35.9 | 359 | 10 | 29.7 |
| 114969 | AW162998 | Hs.24684 | KIAA1376 protein | 9.4 | 94 | 8 | 7.3 |
| 114988 | AA251089 | | gb: zs04f05.s1 NCL_CGAP_GCB1 Homo sapiens | 11.5 | 115 | 1 | 6.9 |
| 115004 | AA329340 | Hs.4867 | mannosyl (alpha-1,3-)-glycoprotein beta- | 4.2 | 42 | 9 | 1.1 |
| 115054 | AW265668 | Hs.87729 | hypothetical protein FLJ12428 | 5.1 | 51 | 1 | 4.2 |
| 115061 | AI751438 | Hs.41271 | Homo sapiens mRNA full length insert cDN | 4.5 | 290 | 65 | 3.7 |
| 115140 | NM_014158 | Hs.279938 | HSPC067 protein | 4.8 | 48 | 1 | 4.4 |
| 115142 | AI623693 | Hs.191533 | ESTs | 3.2 | 49 | 16 | 4.2 |
| 115188 | AK000219 | Hs.88367 | hypothetical protein FLJ20212 | 3.3 | 33 | 1 | 3 |
| 115206 | AW183695 | Hs.186572 | ESTs | 5.8 | 58 | 1 | 5 |
| 115221 | AW365434 | Hs.79741 | hypothetical protein FLJ10116 | 5.5 | 343 | 62 | 2.5 |
| 115262 | AI422867 | Hs.88594 | ESTs | 11.2 | 112 | 1 | 10.3 |
| 115291 | BE545072 | Hs.122579 | hypothetical protein FLJ10461 | 4.5 | 96 | 21 | 7.8 |
| 115536 | AK001468 | Hs.62180 | anillin (Drosophila Scraps homolog), act | 5.9 | 59 | 1 | 4.2 |
| 115583 | NM_012317 | Hs.45231 | leucine zipper, down-regulated in cancer | 9.8 | 98 | 1 | 8.8 |
| 115600 | AA081395 | Hs.42173 | Homo sapiens cDNA FLJ10366 fis, clone NT | 4.6 | 46 | 2 | 1.8 |
| 115622 | AI088691 | Hs.208414 | Homo sapiens mRNA; cDNA DKFZp564D0472 (f | 4.4 | 44 | 7 | 1.1 |
| 115646 | N36110 | Hs.305971 | solute carrier family 2 (facilitated glu | 3.2 | 372 | 115 | 2.1 |
| 115674 | AW992356 | Hs.8364 | Homo sapiens pyruvate dehydrogenase kina | 10.2 | 506 | 50 | 2.8 |
| 115675 | W87707 | Hs.82065 | interleukin 6 signal transducer (gp130, | 5.2 | 405 | 78 | 10.1 |
| 115719 | AW992405 | Hs.59622 | Homo sapiens, clone IMAGE: 3507281, mRNA, | 7.6 | 144 | 19 | 13.9 |
| 115725 | AW899053 | Hs.76917 | F-box only protein 8 | 3.1 | 58 | 19 | 2.5 |
| 115764 | AW582256 | Hs.91011 | anterior gradient 2 (Xenepus laevis) hom | 5.7 | 368 | 65 | 28.5 |
| 115821 | AW338063 | Hs.130965 | zinc-finger protein ZBRK1 | 3.9 | 39 | 8 | 2.2 |
| 115825 | R50956 | Hs.159993 | gycosyltransferase | 4.2 | 79 | 19 | 1.9 |
| 115839 | BE300266 | Hs.28935 | transducin-like enhancer of split 1, hom | 5.8 | 58 | 1 | 4.4 |
| 115844 | AI373062 | Hs.332938 | hypothetical protein MGC5370 | 6.2 | 62 | 1 | 5.4 |
| 115892 | AA291377 | Hs.50831 | ESTs | 3.2 | 40 | 13 | 0.7 |
| 115967 | AI745379 | Hs.42911 | ESTs | 8.4 | 101 | 12 | 8.7 |
| 116093 | AW673312 | Hs.50848 | hypothetical protein FLJ20331 | 3.6 | 36 | 1 | 2 |
| 116097 | AI198719 | Hs.176376 | ESTs | 5.1 | 51 | 1 | 2 |
| 116107 | AL133916 | Hs.172572 | hypothetical protein FLJ20093 | 3.4 | 34 | 8 | 1 |
| 116127 | AF126743 | Hs.279884 | DNAJ domain-containing | 3.5 | 35 | 8 | 3.3 |
| 116129 | AF189011 | Hs.49163 | putative ribonuclease III | 4.5 | 45 | 9 | 3.4 |
| 116204 | AW861622 | Hs.108646 | Homo sapiens cDNA FLJ14934 fis, clone PL | 5.2 | 52 | 4 | 3.9 |
| 116226 | AW976438 | Hs.17428 | RBP1-like protein | 3.8 | 38 | 7 | 2.1 |
| 116238 | AV660717 | Hs.47144 | DKFZP586N0819 protein | 5.1 | 198 | 39 | 17.9 |
| 116250 | N76712 | Hs.44829 | ESTs, Weakly similar to I38022 hypotheti | 13.3 | 133 | 8 | 3.2 |
| 116256 | AA328153 | Hs.88201 | ESTs, Weakly similar to A Chain A, Cryst | 3.3 | 106 | 33 | 9.8 |
| 116298 | AI955411 | Hs.94109 | Homo sapiens cDNA FLJ13634 fis, clone PL | 4.8 | 179 | 38 | 2.8 |
| 116336 | AL133033 | Hs.4084 | KIAA1025 protein | 3.2 | 173 | 55 | 3 |
| 116351 | AL133623 | Hs.82501 | similar to mouse Xm1/Dhm2 protein | 3.7 | 37 | 1 | 1.8 |
| 116365 | N50174 | Hs.46765 | ESTs | 3.9 | 39 | 10 | 0.6 |
| 116379 | AA448588 | Hs.71252 | hypothetical protein DKFZp761C169 | 5.6 | 106 | 19 | 9 |
| 116429 | AF191018 | Hs.279923 | putative nucleotide binding protein, est | 3.6 | 256 | 72 | 3.7 |
| 116450 | AI654450 | Hs.47274 | Homo sapiens mRNA; cDNA DKFZp564B176 (fr | 3.1 | 119 | 39 | 2 |
| 116461 | AA313607 | Hs.58633 | Homo sapiens cDNA: FLJ22145 fis, clone H | 5.5 | 315 | 58 | 3.1 |
| 116470 | AI272141 | Hs.83484 | SRY (sex determining region Y)-box 4 | 3.4 | 496 | 144 | 1.6 |
| 116507 | AI418366 | Hs.68501 | ESTs | 3.1 | 31 | 4 | 1.9 |
| 116579 | AW888411 | Hs.81915 | leukemia-associated phosphoprotein p18 ( | 3.3 | 931 | 279 | 5.6 |
| 116625 | F01601 | Hs.241567 | RNA binding motif, single stranded inter | 3.6 | 36 | 1 | 1.9 |
| 116674 | AI768015 | Hs.92127 | ESTs | 4.5 | 96 | 22 | 6.9 |
| 116680 | AW902848 | Hs.273829 | ESTs | 4.2 | 42 | 1 | 2.7 |
| 116710 | F10577 | Hs.306088 | v-crk avian sarcoma virus CT10 oncogene | 7.1 | 71 | 9 | 6.9 |
| 116724 | AA741307 | Hs.65641 | hypothetical protein FLJ20073 | 4.3 | 190 | 44 | 5.4 |
| 116786 | H25836 | Hs.301527 | ESTs, Moderately similar to unknown [H. s | 22.8 | 228 | 9 | 12.4 |
| 116787 | AW362955 | Hs.15641 | Homo sapiens cDNA FLJ14415 fis, clone HE | 4.9 | 108 | 22 | 9 |
| 116790 | AW161357 | Hs.101174 | microtubule-associated protein tau | 4.6 | 163 | 35 | 7.3 |
| 116844 | H64938 | Hs.337434 | ESTs, Weakly similar to A46010 X-linked | 6.9 | 69 | 10 | 2.4 |
| 117027 | AW085208 | Hs.130093 | ESTs | 4.8 | 48 | 1 | 2.5 |
| 117067 | H91164 | Hs.335797 | ESTs | 3.3 | 33 | 1 | 2.3 |
| 117129 | H95785 | Hs.167652 | ESTs, Highly similar to 1819485A CENP-E | 3.1 | 38 | 13 | 1.7 |
| 117147 | AW901347 | Hs.38592 | hypothetical protein FLJ23342 | 4.8 | 48 | 1 | 0.9 |
| 117170 | N25929 | Hs.42500 | ADP-ribosylation factor-like 5 | 3.1 | 295 | 96 | 27.9 |
| 117209 | W03011 | Hs.306881 | MSTP043 protein | 3.6 | 41 | 12 | 2.8 |
| 117280 | M18217 | Hs.172129 | Homo sapiens cDNA: FLJ21409 fis, clone C | 3.9 | 322 | 83 | 4.4 |
| 117367 | AI041793 | Hs.42502 | ESTs | 3.5 | 72 | 21 | 1.3 |
| 117412 | N32536 | Hs.42645 | solute carrier family 16 (monocarboxylic | 17.4 | 174 | 9 | 6.9 |
| 117475 | N30205 | Hs.93740 | ESTs, Weakly similar to I38022 hypotheti | 3.2 | 35 | 11 | 0.7 |
| 117634 | AW341639 | Hs.13323 | hypothetical protein FLJ22059 | 5 | 50 | 1 | 4.7 |
| 117667 | U59305 | Hs.44708 | Ser-Thr protein kinase related to the my | 4.5 | 211 | 47 | 5 |
| 117852 | AW877787 | Hs.136102 | KIAA0853 protein | 4.6 | 46 | 1 | 3.8 |
| 117873 | N49967 | Hs.46624 | HSPC043 protein | 3.1 | 31 | 1 | 2.7 |
| 117924 | AI521436 | Hs.38891 | ESTs | 4.9 | 49 | 1 | 4.4 |
| 118138 | AA374756 | Hs.93560 | Homo sapiens mRNA for KIAA1771 protein, | 5 | 50 | 2 | 3.1 |
| 118449 | AI813865 | Hs.164478 | hypothetical protein FLJ21939 similar to | 3.6 | 89 | 25 | 0.9 |
| 118467 | AF091434 | Hs.43080 | platelet derived growth factor C | 3.2 | 378 | 117 | 2.8 |
| 118472 | AL157545 | Hs.42179 | bromodomain and PHD finger containing, 3 | 14.5 | 145 | 1 | 2.4 |
| 118475 | N66845 | | gb: za46c11.s1 Soares fetal liver spleen | 3.1 | 199 | 64 | 1 |
| 118509 | N22617 | Hs.43228 | Homo sapiens cDNA FLJ11835 fis, clone HE | 6 | 60 | 5 | 3.7 |
| 118528 | AI949952 | Hs.49397 | ESTs | 3.3 | 81 | 25 | 1.5 |
| 118828 | N79496 | Hs.50824 | EST, Moderately similar to I54374 gene N | 3.4 | 740 | 217 | 2.8 |
| 118836 | AW134482 | Hs.173001 | hypothetical protein FLJ13964 | 4.3 | 162 | 38 | 12.1 |
| 118854 | T58283 | Hs.10450 | Homo sapiens cDNA: FLJ22063 fis, clone H | 3.4 | 118 | 35 | 2.3 |
| 118873 | AI824009 | Hs.44577 | ESTs | 3.5 | 35 | 1 | 2.9 |
| 118888 | AI191811 | Hs.54629 | ESTs | 8.4 | 84 | 10 | 0.8 |
| 118901 | AW292577 | Hs.94445 | ESTs | 7.3 | 73 | 3 | 5.4 |
| 118981 | N29309 | Hs.39288 | ESTs | 5 | 50 | 5 | 4.7 |
| 118991 | NM_016657 | Hs.250696 | KDEL (Lys-Asp-Glu-Leu) endoplasmic retic | 3.7 | 37 | 6 | 0.5 |
| 119023 | N98488 | | gb: zb82h01.s1 Soares_senescent_flbroblas | 3.3 | 36 | 11 | 0.6 |
| 119088 | R39261 | Hs.90790 | Homo sapiens cDNA: FLJ22930 fis, clone K | 3.3 | 167 | 51 | 2.6 |
| 119126 | R45175 | Hs.117183 | ESTs | 5.3 | 53 | 6 | 2.3 |
| 119128 | H09334 | Hs.92482 | ESTs | 3.7 | 37 | 4 | 3 |
| 119271 | AI061118 | Hs.65328 | Fanconi anemia, complementation group F | 8.2 | 82 | 1 | 6.4 |
| 119298 | NM_001241 | Hs.155478 | cyclin T2 | 4 | 40 | 4 | 1.2 |
| 119307 | BE048061 | Hs.37054 | ephrin-A3 | 3.3 | 571 | 171 | 2 |
| 119367 | T78324 | Hs.250895 | ribosomal protein L34 | 3.4 | 34 | 3 | 2.4 |
| 119427 | AW474547 | Hs.53565 | Homo sapiens PIG-M mRNA for mannosyltran | 4.6 | 60 | 13 | 4.8 |
| 119580 | AL079310 | Hs.92260 | high-mobility group protein 2-like 1 | 8.1 | 94 | 12 | 6.5 |
| 119586 | AF088033 | Hs.159225 | ESTs | 3.3 | 33 | 8 | 0.9 |
| 119638 | NM_016122 | Hs.56148 | NY-REN-58 antigen | 3.3 | 33 | 10 | 0.5 |
| 119676 | AA243837 | Hs.57787 | ESTs | 5.4 | 54 | 1 | 4.1 |
| 119717 | AA918317 | Hs.57987 | B-cell CLL/lymphoma 11B (zinc finger pro | 4.6 | 46 | 7 | 0.8 |
| 119771 | AI905687 | Hs.2533 | EST | 3.5 | 2073 | 595 | 2.1 |
| 119780 | NM_016625 | Hs.191381 | hypothetical protein | 4.4 | 44 | 1 | 3.1 |
| 119786 | AL133396 | Hs.121281 | prion protein 2 (dublet) | 3.4 | 34 | 1 | 2.5 |
| 119805 | AJ223810 | Hs.43213 | ESTs, Weakly similar to IEFS_HUMAN TRANS | 3.6 | 36 | 1 | 2.9 |
| 119859 | AW245741 | Hs.58461 | ESTs, Weakly similar to A35659 krueppel- | 5.2 | 52 | 6 | 1.8 |
| 119899 | AI057404 | Hs.58698 | ESTs | 3.7 | 37 | 4 | 1.9 |
| 119940 | AL050097 | Hs.272531 | DKFZP586B0319 protein | 6.9 | 162 | 24 | 2.6 |
| 119943 | BE565849 | Hs.14158 | copine III | 3.7 | 590 | 159 | 3.8 |
| 120132 | W57554 | Hs.125019 | lymphoid nuclear protein (LAF-4) mRNA | 6.9 | 319 | 47 | 2.1 |
| 120150 | BE005771 | Hs.153746 | hypothetical protein FLJ22490 | 5.3 | 53 | 5 | 0.9 |
| 120215 | AF109219 | Hs.108787 | phosphatidylinositol glycan, class N | 3.2 | 106 | 34 | 3.3 |
| 120260 | AK000061 | Hs.101590 | hypothetical protein | 3.4 | 34 | 1 | 1.7 |
| 120296 | AW995911 | Hs.299883 | hypothetical protein FLJ23399 | 4.2 | 124 | 30 | 1.8 |
| 120352 | R06859 | Hs.193172 | ESTs, Weakly similar to I38022 hypotheti | 7.5 | 112 | 15 | 2.5 |
| 120378 | AA223249 | Hs.285728 | abl-interactor 12 (SH3-containing protei | 3.3 | 33 | 10 | 2.8 |
| 120418 | AW966893 | Hs.26613 | Homo sapiens mRNA; cDNA DKFZp586F1323 (f | 4.8 | 48 | 1 | 0.5 |
| 120473 | AA251973 | Hs.269988 | ESTs | 3.4 | 34 | 4 | 0.1 |
| 120493 | AW968080 | Hs.152939 | Homo sapiens clone 24630 mRNA sequence | 3.9 | 161 | 42 | 2 |
| 120524 | AA261852 | Hs.192905 | ESTs | 6.8 | 68 | 1 | 0.2 |
| 120554 | AA284447 | Hs.271887 | ESTs | 3.2 | 32 | 5 | 0.6 |
| 120562 | BE244580 | Hs.302267 | hypothetical protein FLJ10330 | 8.5 | 127 | 15 | 1.6 |
| 120571 | AB037744 | Hs.34892 | KIAA1323 protein | 3.7 | 37 | 1 | 0.5 |
| 120572 | H39599 | Hs.294008 | ESTs | 3.6 | 36 | 8 | 0.2 |
| 120588 | AA703226 | Hs.16193 | Homo sapiens mRNA; cDNA DKFZp586B211 (fr | 5.6 | 101 | 18 | 1.6 |
| 120649 | AA687322 | Hs.192843 | leucine zipper protein FKSG14 | 5.4 | 54 | 10 | 2.5 |
| 120658 | AI952639 | Hs.98267 | ESTs | 3.2 | 32 | 8 | 3 |
| 120713 | AW449855 | Hs.96557 | Homo sapiens cDNA FLJ12727 fis, clone NT | 5.3 | 58 | 11 | 3.3 |
| 120821 | Y19062 | Hs.96870 | staufen (Drosophila, RNA-binding protein | 3.3 | 33 | 3 | 0.2 |
| 120822 | AA347422 | Hs.238040 | EST, Weakly similar to B34087 hypothetic | 3.8 | 38 | 7 | 0.2 |
| 120915 | AL135556 | Hs.97104 | ESTs | 3.5 | 37 | 11 | 0.1 |
| 120922 | AA481003 | Hs.97128 | ESTs | 3.1 | 31 | 1 | 0.4 |
| 120977 | AA398155 | Hs.97600 | ESTs | 7.9 | 79 | 1 | 2.7 |
| 120999 | AI972375 | Hs.29626 | hypothetical brain protein my038 | 5.1 | 51 | 1 | 2.4 |
| 121125 | AL042981 | Hs.251278 | KIAA1201 protein | 3.7 | 37 | 10 | 1 |
| 121176 | AL121523 | Hs.97774 | ESTs | 7 | 70 | 1 | 0.9 |
| 121202 | AA970946 | Hs.97794 | ESTs | 3.9 | 39 | 1 | 0.2 |
| 121429 | AA406293 | Hs.41167 | ESTs | 3.4 | 34 | 1 | 0.8 |
| 121448 | AF044197 | Hs.100431 | B-cell attracting chemokine 1 (CXCL13; | 3.5 | 35 | 1 | 2.6 |
| 121463 | AK000282 | Hs.239681 | hypothetical protein FLJ20275 | 10.3 | 103 | 1 | 9.3 |
| 121517 | AI002968 | Hs.235402 | ESTs, Weakly similar to T26525 hypotheti | 3.5 | 143 | 41 | 2.6 |
| 121553 | AA412488 | Hs.48820 | TATA box binding protein (TBP)-associate | 4.6 | 46 | 3 | 0.8 |
| 121556 | AA412494 | Hs.98152 | EST | 4.2 | 77 | 19 | 1.4 |
| 121581 | AA416568 | | gb: zu05c10.s1 Soares_testis_NHT Homo sap | 3.2 | 32 | 1 | 0.8 |
| 121709 | AI338247 | Hs.98314 | Homo sapiens mRNA; cDNA DKFZp586L0120 (f | 3.4 | 34 | 10 | 0.7 |
| 121723 | AA243499 | Hs.104800 | hypothetical protein FLJ10134 | 2.9 | 214 | 74 | 3.7 |
| 121831 | AA449644 | Hs.193063 | Homo sapiens cDNA FLJ14201 fis, clone NT | 3.9 | 39 | 1 | 0.2 |
| 121853 | AA425887 | Hs.98502 | hypothetical protein FLJ14303 | 4.4 | 48 | 11 | 0.9 |
| 121873 | AV650929 | Hs.145696 | splicing factor (CC1.3) | 3.6 | 150 | 42 | 3.2 |
| 121913 | AI249368 | Hs.98558 | ESTs; protease inhibitor 15 (PI15) | 2.7 | 864 | 321 | 0.6 |
| 121916 | AW117207 | Hs.98523 | ESTs | 3.5 | 35 | 3 | 2.3 |
| 122004 | AI810721 | Hs.95424 | ESTs | 4.9 | 49 | 7 | 3.7 |
| 122063 | AW794215 | Hs.301226 | KIAA1085 protein | 3.2 | 88 | 28 | 1.2 |
| 122223 | AF169797 | Hs.27413 | adaptor protein containing pH domain, PT | 12.6 | 126 | 7 | 7.5 |
| 122235 | AA436475 | Hs.112227 | membrane-associated nucleic acid binding | 4.1 | 43 | 11 | 1.6 |
| 122273 | AI298368 | Hs.150926 | fucose-1-phosphate guanylyltransferase | 3.1 | 31 | 1 | 1 |
| 122383 | AA446189 | Hs.99051 | ESTs | 3.3 | 53 | 16 | 4 |
| 122507 | BE567620 | Hs.99210 | ESTs | 3.2 | 291 | 91 | 4 |
| 122524 | AA449453 | Hs.192915 | ESTs, Weakly similar to ALU1_HUMAN ALU S | 3.1 | 31 | 6 | 0.8 |
| 122636 | AW651706 | Hs.99519 | hypothetical protein FLJ14007 | 3.5 | 35 | 1 | 3 |
| 122637 | AA454149 | Hs.99357 | EST | 3.2 | 32 | 10 | 3.1 |
| 122798 | AW366286 | Hs.145696 | splicing factor (CC1.3) | 3.2 | 36 | 11 | 2.5 |
| 122861 | AA335721 | Hs.119394 | ESTs | 5.6 | 108 | 20 | 1.8 |
| 122873 | AA749382 | Hs.118797 | ubiquitin-conjugating enzyme E2D 3 (homo | 3.6 | 36 | 1 | 3.4 |
| 122946 | AI718702 | Hs.308026 | major histocompatibility complex, class | 3.7 | 162 | 44 | 12.4 |
| 122963 | AA478446 | Hs.69559 | KIAA1096 protein | 7.2 | 72 | 1 | 5.7 |
| 122974 | AA447871 | Hs.194215 | ESTs, Weakly similar to I38022 hypotheti | 4.7 | 59 | 13 | 4.7 |
| 123016 | AW338067 | Hs.323231 | Homo sapiens cDNA FLJ11946 fis, clone HE | 3.3 | 207 | 63 | 3.5 |
| 123090 | AL135185 | Hs.48778 | niban protein | 3.8 | 207 | 55 | 5.5 |
| 123137 | AI073913 | Hs.100686 | ESTs, Weakly similar to JE0350 Anterior | 9.9 | 351 | 36 | 13.9 |
| 123255 | AA830335 | Hs.105273 | ESTs | 4.1 | 72 | 18 | 1.5 |
| 123284 | AA488988 | Hs.293796 | ESTs | 3.7 | 41 | 11 | 1.6 |
| 123442 | AA299652 | Hs.111496 | Homo sapiens cDNA FLJ11643 fis, clone HE | 6.7 | 67 | 2 | 2.1 |
| 123449 | AL049325 | Hs.112493 | Homo sapiens mRNA; cDNA DKFZp564D036 (fr | 3.4 | 34 | 1 | 2.6 |
| 123475 | BE439553 | Hs.250528 | Homo sapiens, clone IMAGE: 4098694, mRNA, | 9.7 | 102 | 11 | 6 |
| 123494 | AW179019 | Hs.112110 | mitochondrial ribosomal protein L42 | 4.2 | 42 | 7 | 2.9 |
| 123503 | AW975051 | Hs.293156 | ESTs, Weakly similar to I78885 serine/th | 3.9 | 39 | 1 | 3.2 |
| 123516 | AB037860 | Hs.173933 | nuclear factor I/A | 4.3 | 43 | 1 | 3.5 |
| 123518 | AL035414 | Hs.21068 | hypothetical protein | 5.8 | 58 | 1 | 4.9 |
| 123523 | AA608588 | | gb: ae54e06.s1 Stratagene lung carcinoma | 3.1 | 927 | 295 | 2.1 |
| 123527 | AF150208 | Hs.108327 | damage-specific DNA binding protein 1 (1 | 5 | 121 | 25 | 5.9 |
| 123570 | AA608955 | Hs.109653 | ESTs | 6.8 | 68 | 10 | 6.1 |
| 123619 | AA602964 | | gb: no97c02.s1 NCI_CGAP_Pr2 Homo sapiens | 8.5 | 85 | 1 | 4.3 |
| 123673 | BE550112 | Hs.158549 | ESTs, Weakly similar to T2D3_HUMAN TRANS | 3.9 | 39 | 5 | 3.7 |
| 123709 | AA706910 | Hs.112742 | ESTs | 3.9 | 60 | 16 | 4.8 |
| 123926 | AA425769 | Hs.227933 | Alg5, S. cerevisiae, homolog of | 3.4 | 80 | 24 | 3.8 |
| 123960 | AW082862 | Hs.287733 | hypothetical protein FLJ23189 | 4.5 | 45 | 2 | 3.6 |
| 124006 | AI147155 | Hs.270016 | ESTs | 5.8 | 321 | 55 | 17 |
| 124059 | BE387335 | Hs.283713 | ESTs, Weakly similar to S64054 hypotheti | 10.4 | 880 | 85 | 5.3 |
| 124287 | H88296 | Hs.5123 | inorganic pyrophosphatase | 3.1 | 41 | 14 | 2.7 |
| 124292 | H11341 | Hs.13366 | Homo sapiens cDNA: FLJ23567 fis, clone L | 3.2 | 32 | 1 | 1.5 |
| 124308 | AA249027 | Hs.241507 | ribosomal protein S6 | 10.5 | 105 | 1 | 9.9 |
| 124315 | NM_005402 | Hs.288757 | v-ral simian leukemia viral oncogene hom | 12.8 | 141 | 11 | 12.2 |
| 124461 | AF283776 | Hs.80285 | Homo sapiens mRNA; cDNA DKFZp586C1723 (f | 3.1 | 31 | 1 | 1.8 |
| 124483 | AI821780 | Hs.179864 | ESTs | 3.3 | 33 | 1 | 1.7 |
| 124677 | R01073 | | gb: ye84c03.s1 Soares fetal liver spleen | 4.2 | 42 | 7 | 3 |
| 124777 | R41933 | Hs.140237 | ESTs, Weakly similar to ALU1_HUMAN ALU S | 3.4 | 210 | 63 | 3.3 |
| 124940 | AF068846 | Hs.103804 | heterogeneous nuclear ribonucleoprotein | 6.5 | 162 | 25 | 14.7 |
| 125079 | T90298 | Hs.271396 | ESTs | 3.1 | 31 | 6 | 2.4 |
| 125091 | T91518 | | gb: ye20f05.s1 Stratagene lung (937210) H | 3.4 | 985 | 286 | 2.8 |
| 125103 | AA570056 | Hs.122730 | ESTs, Moderately similar to KIAA1215 pro | 3.6 | 224 | 63 | 4 |
| 125144 | AB037742 | Hs.24336 | KIAA1321 protein | 6.3 | 63 | 6 | 5 |
| 125150 | W38240 | | Empirically selected from AFFX single pr | 3.6 | 38 | 11 | 2.6 |
| 125156 | W93048 | Hs.250723 | hypothetical protein MGC2747 | 3.1 | 31 | 1 | 2.8 |
| 125226 | AA782536 | Hs.122647 | N-myristoyltransferase 2 | 3.2 | 37 | 12 | 3.6 |
| 125279 | AW401809 | Hs.4779 | KIAA1150 protein | 13.1 | 131 | 1 | 5.1 |
| 125299 | T32982 | Hs.102720 | ESTs | 7.7 | 81 | 11 | 7.6 |
| 125303 | AA173319 | Hs.288193 | hypothetical protein MGC12217 | 14.3 | 143 | 9 | 13.1 |
| 125377 | W72949 | Hs.77495 | UBX domain-containing 1 | 3.3 | 34 | 11 | 3.2 |
| 125390 | AL038165 | Hs.75187 | translocase of outer mitochondrial membr | 8.2 | 124 | 15 | 11.5 |
| 125471 | AA421691 | Hs.152601 | UDP-glucose ceramide glucosyltransferase | 3.7 | 224 | 61 | 21 |
| 125617 | AA287921 | Hs.164950 | ESTs | 6.7 | 67 | 1 | 6 |
| 125621 | T62641 | Hs.278544 | acetyl-Coenzyme A acetyltransferase 2 (a | 5.5 | 55 | 10 | 4.2 |
| 125628 | AA418069 | Hs.241493 | natural killer-tumor recognition sequenc | 5.5 | 63 | 12 | 1 |
| 125660 | AW292171 | Hs.23978 | scaffold attachment factor B | 4.3 | 68 | 16 | 2.8 |
| 125698 | AF078847 | Hs.191356 | general transcription factor IIH, polype | 4.8 | 48 | 5 | 4.1 |
| 125745 | AI858032 | Hs.75722 | ribophorin II | 6.8 | 223 | 33 | 2.8 |
| 125770 | AA143045 | Hs.81665 | v-kit Hardy-Zuckerman 4 feline sarcoma v | 8.3 | 87 | 11 | 0.4 |
| 125827 | NM_003403 | Hs.97496 | YY1 transcription factor | 11.3 | 124 | 11 | 9.7 |
| 125852 | AW630088 | Hs.76550 | Homo sapiens mRNA; cDNA DKFZp564B1264 (f | 30.6 | 306 | 4 | 26.5 |
| 126349 | T30968 | Hs.13531 | hypothetical protein FLJ10971 | 4.9 | 68 | 14 | 1.4 |
| 126384 | AW090198 | Hs.4779 | KIAA1150 protein | 6.4 | 74 | 12 | 6.6 |
| 126590 | W78968 | Hs.181307 | H3 histone, family 3A | 5 | 264 | 53 | 3.4 |
| 126645 | AA316181 | Hs.61635 | six transmembrane epithelial antigen of | 3.8 | 38 | 1 | 2.7 |
| 126663 | AW518478 | Hs.181297 | ESTs | 3.6 | 36 | 6 | 2.9 |
| 126695 | AA643322 | Hs.172028 | a disintegrin and metalloproteinase doma | 3.1 | 31 | 1 | 2.5 |
| 126764 | AA036755 | Hs.102178 | syntaxin 16 | 4.4 | 76 | 18 | 1 |
| 126801 | AW663887 | Hs.7337 | hypothetical protein FUJ10936 | 3.8 | 38 | 1 | 3 |
| 126813 | AW163483 | Hs.48320 | double ring-finger protein, Dorfin | 6.7 | 155 | 23 | 1.4 |
| 126838 | AL043489 | Hs.279609 | mitochondrial carrier homolog 2 | 8.8 | 110 | 13 | 10.5 |
| 126855 | AA129640 | Hs.128065 | ESTs | 3.6 | 36 | 10 | 1.9 |
| 126971 | T26989 | Hs.283664 | aspartate beta-hydroxylase | 5.5 | 79 | 15 | 4.4 |
| 127167 | AA625690 | Hs.190272 | ESTs | 3.1 | 33 | 11 | 2.3 |
| 127251 | AA936428 | Hs.128638 | ESTs | 3.5 | 35 | 1 | 3.1 |
| 127349 | AA412108 | Hs.269350 | ESTs | 4.8 | 106 | 22 | 1 |
| 127439 | D60237 | Hs.14368 | SH3 domain binding glutamic acid-rich pr | 7.5 | 75 | 1 | 6.5 |
| 127537 | AI926047 | Hs.162859 | ESTs | 3.8 | 38 | 7 | 3.4 |
| 127542 | AA703684 | Hs.245474 | ESTs, Moderately similar to ALU5_HUMAN A | 3.3 | 33 | 9 | 0.9 |
| 127677 | AF175265 | Hs.264190 | vacuolar protein sorting 35 (yeast homol | 4.3 | 152 | 35 | 12.5 |
| 127774 | AA313639 | Hs.119488 | cystein-rich hydrophobic domain 2 | 5.4 | 73 | 14 | 6.8 |
| 127999 | AW978827 | Hs.69851 | nucleolar protein family A, member 1 (H/ | 5.2 | 81 | 16 | 1.1 |
| 128218 | AA186733 | Hs.292154 | stromal cell protein | 3.9 | 220 | 57 | 2.5 |
| 128305 | AI954968 | Hs.279009 | matrix Gla protein | 9.4 | 94 | 3 | 5.3 |
| 128470 | AL049974 | Hs.100261 | Homo sapiens mRNA; cDNA DKFZp564B222 (fr | 4.6 | 46 | 8 | 3.9 |
| 128482 | AI694143 | Hs.296251 | programmed cell death 4 | 7.2 | 72 | 1 | 5.8 |
| 128501 | AL133572 | Hs.199009 | protein containing CXXC domain 2 | 3.8 | 38 | 1 | 0.9 |
| 128517 | AW994403 | Hs.100861 | hypothetical protein FLJ14600 | 5.6 | 73 | 13 | 6.1 |
| 128530 | AI932995 | Hs.183475 | Homo sapiens clone 25061 mRNA sequence | 4.2 | 104 | 25 | 7.8 |
| 128579 | N25956 | Hs.101810 | Homo sapiens cDNA FLJ14232 fis, clone NT | 3.1 | 172 | 55 | 3.1 |
| 128595 | U31875 | Hs.272499 | short-chain alcohol dehydrogenase family | 3.3 | 105 | 32 | 3 |
| 128610 | N48373 | Hs.10247 | activated leucocyte cell adhesion molecu | 7.3 | 106 | 15 | 5 |
| 128653 | D87432 | Hs.10315 | solute carrier family 7 (cationic amino | 3.1 | 31 | 1 | 2.2 |
| 128742 | AA307211 | Hs.251531 | proteasome (prosome, macropain) subunit, | 3.6 | 130 | 36 | 3.5 |
| 128773 | NM_004131 | Hs.1051 | granzyme B (granzyme 2, cytotoxic T-lymp | 3.9 | 43 | 11 | 1.8 |
| 128790 | AF026692 | Hs.105700 | secreted frizzled-related protein 4 | 17.4 | 409 | 24 | 7.8 |
| 128793 | AB011125 | Hs.105749 | KIAA0553 protein | 3.1 | 34 | 11 | 2.7 |
| 128794 | NM_014720 | Hs.105751 | Ste20-related serine/threonine kinase | 3.6 | 36 | 5 | 1.5 |
| 128835 | AK001731 | Hs.106390 | Homo sapiens mRNA; cDNA DKFZp586H0924 (f | 3.3 | 288 | 87 | 7.9 |
| 128906 | R57988 | Hs.10706 | epithelial protein lost in neoplasm beta | 11.3 | 113 | 8 | 2.5 |
| 128925 | R67419 | Hs.21851 | Homo sapiens cDNA FLJ12900 fis, clone NT | 7.1 | 392 | 56 | 3.6 |
| 128949 | AA009647 | Hs.8850 | a disintegrin and metalloproteinase doma | 4.6 | 132 | 29 | 9.7 |
| 129017 | AA115333 | Hs.107968 | ESTs | 8.2 | 82 | 1 | 7.4 |
| 129075 | BE250162 | Hs.83765 | dihydrofolate reductase | 5 | 50 | 1 | 3.3 |
| 129095 | L12350 | Hs.108623 | thrombospondin 2 | 3.2 | 814 | 257 | 2.4 |
| 129151 | N23018 | Hs.171391 | C-terminal binding protein 2 | 4.4 | 44 | 1 | 3.8 |
| 129168 | AI132988 | Hs.109052 | chromosome 14 open reading frame 2 | 14.2 | 142 | 6 | 9.4 |
| 129229 | AF013758 | Hs.109643 | polyadenylate binding protein-interactin | 7.1 | 71 | 1 | 6.2 |
| 129243 | BE169531 | Hs.109727 | TAK1-binding protein 2; KIAA0733 protein | 5 | 64 | 13 | 6.3 |
| 129259 | AF220050 | Hs.181385 | uncharacterized hematopoietic stem/proge | 5.2 | 75 | 15 | 6.4 |
| 129278 | NM_015344 | Hs.11000 | leptin receptor overlapping transcript-I | 3.7 | 39 | 11 | 3.2 |
| 129337 | NM_014918 | Hs.110488 | KIAA0990 protein | 9.5 | 95 | 1 | 8.5 |
| 129351 | AL049538 | Hs.62349 | ras association (RaIGDS/AF-6) domain con | 7.6 | 92 | 12 | 1.4 |
| 129366 | BE220806 | Hs.184697 | Homo sapiens clone 23785 mRNA sequence | 7.1 | 150 | 21 | 14.5 |
| 129393 | BE219987 | Hs.166982 | phosphatidylinositol glycan, class F | 3.9 | 54 | 14 | 5.1 |
| 129457 | X61959 | Hs.207776 | aspartylglucosaminidase | 3.6 | 36 | 1 | 2.7 |
| 129486 | NM_005754 | Hs.220689 | Ras-GTPase-activating protein SH3-domain | 4 | 40 | 4 | 3.2 |
| 129586 | AW964541 | Hs.11500 | hypothetical protein FLJ21127 | 4.6 | 199 | 44 | 2.3 |
| 129598 | N30436 | Hs.11556 | Homo sapiens cDNA FLJ12566 fis, clone NT | 4.2 | 42 | 1 | 3.8 |
| 129691 | M26939 | Hs.119571 | collagen, type III, alpha 1 (Ehlers-Danl | 6.4 | 1111 | 175 | 5 |
| 129698 | BE242144 | Hs.12013 | ATP-binding cassette, sub-family E (OABP | 4.8 | 48 | 8 | 3.8 |
| 129721 | NM_001415 | Hs.211539 | eukaryotic translation initiation factor | 5.8 | 171 | 30 | 2.9 |
| 129740 | BE165866 | Hs.83623 | nuclear receptor subfamily 1, group I, m | 4.5 | 45 | 1 | 2.4 |
| 129755 | R42216 | Hs.12342 | Homo sapiens clone 24538 mRNA sequence | 5.3 | 53 | 9 | 3.6 |
| 129801 | R39246 | Hs.239666 | Homo sapiens cDNA FLJ13495 fis, clone PL | 3.1 | 31 | 2 | 2.5 |
| 129821 | AB028945 | Hs.12696 | cortactin SH3 domain-binding protein | 11.4 | 114 | 1 | 10 |
| 129869 | AI222069 | Hs.13015 | hypothetical protein similar to mouse Dn | 4.7 | 556 | 119 | 4.5 |
| 129965 | T71333 | Hs.13854 | ESTs | 3.1 | 31 | 3 | 3 |
| 129977 | NM_000399 | Hs.1395 | early growth response 2 (Krox-20 (Drosop | 3.2 | 32 | 1 | 0.2 |
| 130036 | BE061916 | Hs.125849 | chromosome 8 open reading frame 2 | 6.7 | 67 | 1 | 5.7 |
| 130057 | AF027153 | Hs.324787 | solute carrier family 5 (inositol transp | 1 | 1 | 1 | 1 |
| 130095 | AK001635 | Hs.14838 | hypothetical protein FLJ10773 | 14.6 | 219 | 15 | 7.6 |
| 130115 | T47294 | Hs.149923 | X-box binding protein 1 | 3.1 | 1336 | 434 | 1.4 |
| 130170 | AW977534 | Hs.151469 | calcium/calmodulin-dependent serine prot | 5.3 | 53 | 9 | 3.2 |
| 130173 | U38847 | Hs.151518 | TAR (HIV) RNA-binding protein 1 | 4.2 | 46 | 11 | 1.1 |
| 130343 | AB040914 | Hs.278628 | KIAA1481 protein | 13.2 | 331 | 25 | 12.4 |
| 130356 | AF127577 | Hs.155017 | nuclear receptor interacting protein 1 | 3.3 | 354 | 108 | 4 |
| 130367 | AL135301 | Hs.8768 | hypothetical protein FLJ10849 | 8.1 | 81 | 9 | 5.5 |
| 130385 | AW067800 | Hs.155223 | stanniocalcin 2 | 72.2 | 722 | 1 | 1.9 |
| 130407 | BE385099 | Hs.334727 | hypothetical protein MGC3017 | 6.5 | 65 | 4 | 5.3 |
| 130417 | AW163518 | Hs.155485 | huntingtin interacting protein 2 | 3.5 | 79 | 23 | 2.5 |
| 130441 | U63630 | Hs.155637 | protein kinase, DNA-activated, catalytic | 6.1 | 61 | 1 | 5.7 |
| 130455 | D90041 | Hs.155956 | N-acetyltransferase 1 (arylamine N-acety | 10.8 | 706 | 66 | 9.2 |
| 130466 | W19744 | Hs.180059 | Homo sapiens cDNA FLJ20653 fis, clone KA | 3.9 | 39 | 1 | 1.9 |
| 130526 | AW876523 | Hs.15929 | hypothetical protein FLJ12910 | 3.9 | 39 | 1 | 2.6 |
| 130567 | AA383092 | Hs.1608 | replication protein A3 (14 kD) | 4.4 | 44 | 1 | 4.1 |
| 130604 | AA383256 | Hs.1657 | estrogen receptor 1 | 32.2 | 322 | 1 | 4.7 |
| 130614 | AI354355 | Hs.16697 | down-regulator of transcription 1, TBP-b | 5.2 | 251 | 48 | 21 |
| 130617 | M90516 | Hs.1674 | glutamine-fructose-6-phosphate transamin | 10 | 100 | 1 | 7.6 |
| 130619 | AI963376 | Hs.12532 | chromosome 1 open reading frame 21 | 3.9 | 39 | 1 | 3.4 |
| 130625 | AF176012 | Hs.260720 | J domain containing protein 1 | 10.5 | 105 | 1 | 9 |
| 130677 | AL161961 | Hs.17767 | KIAA1554 protein | 6.8 | 129 | 19 | 12.1 |
| 130681 | R62676 | Hs.17820 | Rho-associated, coiled-coil containing p | 4.1 | 41 | 1 | 3.6 |
| 130693 | R68537 | Hs.17962 | ESTs | 9.2 | 234 | 26 | 16.8 |
| 130712 | AJ271881 | Hs.279762 | bromodomain-containing 7 | 17.5 | 175 | 2 | 12.8 |
| 130723 | BE247676 | Hs.18442 | E-1 enzyme | 8.1 | 81 | 3 | 2.8 |
| 130751 | AF052105 | Hs.18879 | chromosome 12 open reading frame | 4.9 | 49 | 1 | 4.3 |
| 130780 | AA197226 | Hs.19347 | hypothetical protein MGC11321 | 3.6 | 100 | 28 | 6.6 |
| 130863 | Y10805 | Hs.20521 | HMT1 (hnRNP methyltransferase, S. cerevi | 3.4 | 525 | 154 | 5.3 |
| 130871 | AF080158 | Hs.226573 | inhibitor of kappa light polypeptide gen | 10.5 | 121 | 12 | 1.6 |
| 130888 | AL044315 | Hs.173094 | Homo sapiens mRNA for KIAA1750 protein, | 6 | 202 | 34 | 3.7 |
| 130974 | NM_003528 | Hs.2178 | H2B histone family, member Q | 7.1 | 100 | 14 | 7.5 |
| 130979 | NM_012446 | Hs.169833 | single-stranded-DNA-binding protein | 3.2 | 87 | 27 | 1.7 |
| 130987 | BE613269 | Hs.21893 | hypothetical protein DKFZp761N0624 | 3.5 | 124 | 35 | 6.5 |
| 130993 | T97401 | Hs.21929 | ESTs | 4.5 | 45 | 1 | 2.5 |
| 131076 | AA749230 | Hs.26433 | dolichyl-phosphate (UDP-N-acetylglucosam | 3.2 | 210 | 66 | 3.8 |
| 131085 | BE207357 | Hs.3454 | KIAA1821 protein | 3.8 | 42 | 11 | 0.6 |
| 131126 | NM_016156 | Hs.181326 | KIAA1073 protein | 6.7 | 67 | 6 | 1.9 |
| 131129 | BE541042 | Hs.23240 | Homo sapiens cDNA: FLJ21848 fis, clone H | 5.8 | 115 | 20 | 2.5 |
| 131148 | AW953575 | Hs.303125 | p53-induced protein PIGPC1 | 3.8 | 585 | 153 | 3.7 |
| 131164 | AW013807 | Hs.182265 | keratin 19 | 5.2 | 1320 | 256 | 3.2 |
| 131176 | AA465113 | Hs.23853 | ESTs, Weakly similar to A34615 profilagg | 3.8 | 38 | 1 | 3.3 |
| 131200 | BE540516 | Hs.293732 | hypothetical protein MGC3195 | 4.8 | 48 | 1 | 4.1 |
| 131216 | AI815486 | Hs.243901 | Homo sapiens cDNA FLJ20738 fis, clone HE | 6.1 | 343 | 56 | 16.4 |
| 131245 | AL080080 | Hs.24766 | thioredoxin domain-containing | 8 | 100 | 13 | 2.9 |
| 131248 | AI038989 | Hs.332633 | Bardet-Biedl syndrome 2 | 4 | 95 | 24 | 1.1 |
| 131273 | AW206008 | Hs.283378 | Homo sapiens cDNA: FLJ21778 fis, clone H | 4.6 | 239 | 53 | 3.5 |
| 131319 | NM_003155 | Hs.25590 | stanniocalcin 1 | 3.5 | 402 | 114 | 2.1 |
| 131367 | AI750575 | Hs.173933 | nuclear factor I/A | 3.3 | 775 | 233 | 2.4 |
| 131375 | AW293165 | Hs.143134 | ESTs | 3.8 | 38 | 1 | 3 |
| 131379 | AK001123 | Hs.26176 | hypothetical protein FLJ10261 | 3.9 | 116 | 30 | 0.5 |
| 131388 | NM_014810 | Hs.92200 | KIAA0480 gene product | 7.6 | 76 | 1 | 5 |
| 131475 | AA992841 | Hs.27263 | KIAA1458 protein | 5.1 | 113 | 22 | 6.1 |
| 131492 | AI452601 | Hs.288869 | nuclear receptor subfamily 2, group F, m | 8.4 | 169 | 20 | 4.6 |
| 131501 | AV661958 | Hs.8207 | GK001 protein | 3.1 | 197 | 63 | 18.7 |
| 131535 | N22120 | Hs.75277 | hypothetical protein FLJ13910 | 5.9 | 59 | 1 | 4.4 |
| 131544 | AL355715 | Hs.28555 | programmed cell death 9 (PDCD9) | 5.1 | 51 | 1 | 3.9 |
| 131546 | AA093668 | Hs.28578 | muscleblind (Drosophila)-like | 3.8 | 79 | 21 | 6.9 |
| 131562 | NM_003512 | Hs.28777 | H2A histone family, member L | 4 | 350 | 88 | 3 |
| 131564 | T93500 | Hs.28792 | Homo sapiens cDNA FLJ11041 fis, clone PL | 4.7 | 381 | 81 | 6.4 |
| 131604 | AA306477 | Hs.29379 | hypothetical protein FLJ10687 | 4.6 | 46 | 7 | 3.8 |
| 131684 | NM_002104 | Hs.3066 | granzyme K (serine protease, granzyme 3; | 3.2 | 82 | 26 | 6.6 |
| 131687 | BE297635 | Hs.3069 | heat shock 70 kD protein 9B (mortalin-2) | 6.7 | 93 | 14 | 8.4 |
| 131689 | AB012124 | Hs.30696 | transcription factor-like 5 (basic helix | 3.8 | 51 | 14 | 1.7 |
| 131693 | AW963776 | Hs.110796 | SAR1 protein | 7.2 | 72 | 4 | 5.7 |
| 131739 | AF017986 | Hs.31386 | secreted frizzled-related protein 2 | 2.1 | 1561 | 757 | 1.7 |
| 131742 | AA961420 | Hs.31433 | ESTs | 11.7 | 117 | 1 | 10.1 |
| 131775 | AB014548 | Hs.31921 | KIAA0648 protein | 4.8 | 48 | 1 | 4.6 |
| 131787 | D87077 | Hs.196275 | KIAA0240 protein | 3.2 | 207 | 64 | 5.5 |
| 131798 | X86098 | Hs.301449 | adenovirus 5 E1A binding protein | 3.4 | 115 | 34 | 9.1 |
| 131836 | W00712 | Hs.32990 | DKFZP566F084 protein | 5.8 | 91 | 16 | 1.4 |
| 131853 | AI681917 | Hs.3321 | ESTs, Highly similar to IRX1_HUMAN IROQU | 4.9 | 632 | 129 | 1.7 |
| 131877 | J04088 | Hs.156346 | topoisomerase (DNA) II alpha (170 kD) | 6.8 | 68 | 1 | 5.6 |
| 131881 | AW361018 | Hs.3383 | upstream regulatory element binding prot | 4 | 140 | 35 | 1.8 |
| 131885 | BE502341 | Hs.3402 | ESTs | 5.7 | 57 | 1 | 4.5 |
| 131904 | AF078866 | Hs.284296 | Homo sapiens cDNA: FLJ22993 fis, clone K | 5.5 | 90 | 17 | 2.9 |
| 131919 | T15803 | Hs.272458 | protein phosphatase 3 (formerly 2B), cat | 5.6 | 95 | 17 | 9.1 |
| 131941 | BE252983 | Hs.35086 | ubiquitin specific protease 1 | 7.4 | 103 | 14 | 6.5 |
| 131945 | NM_002916 | Hs.35120 | replication factor C (activator 1) 4 (37 | 3.7 | 37 | 1 | 3.4 |
| 131949 | AK000010 | Hs.258798 | hypothetical protein FLJ20003 | 3.5 | 35 | 1 | 2.5 |
| 131965 | W79283 | Hs.35962 | ESTs | 5.5 | 168 | 31 | 4.4 |
| 131977 | U90441 | Hs.3622 | procollagen-proline, 2-oxoglutarate 4-di | 3.7 | 37 | 9 | 2.8 |
| 131985 | AA503020 | Hs.36563 | hypothetical protein FLJ22418 | 40.2 | 402 | 1 | 4 |
| 131993 | AI878910 | Hs.3688 | cisplatin resistance-associated overexpr | 7.3 | 73 | 1 | 1.2 |
| 132064 | AA121098 | Hs.3838 | serum-inducible kinase | 22.6 | 226 | 10 | 0.9 |
| 132094 | NM_016045 | Hs.3945 | CGI-107 protein | 3.1 | 227 | 73 | 16.8 |
| 132109 | AW190902 | Hs.40098 | cysteine knot superfamily 1, BMP antagon | 3.5 | 73 | 21 | 6.3 |
| 132116 | AW960474 | Hs.40289 | ESTs | 3.6 | 141 | 39 | 12.6 |
| 132143 | D52059 | Hs.7972 | KIAA0871 protein | 4.9 | 49 | 1 | 4.1 |
| 132160 | W26406 | Hs.295923 | seven in absentia (Drosophila) homolog 1 | 4.4 | 53 | 12 | 2.1 |
| 132164 | AI752235 | Hs.41270 | procellagen-lysine, 2-oxoglutarate 5-dio | 5 | 225 | 45 | 9.1 |
| 132180 | NM_004460 | Hs.418 | fibroblast activation protein, alpha | 10.7 | 433 | 41 | 7.2 |
| 132197 | AI699482 | Hs.42151 | ESTs | 3.4 | 58 | 17 | 4 |
| 132256 | AI078645 | Hs.431 | murine leukemia viral (bmi-1) oncogene h | 4.2 | 42 | 1 | 2.2 |
| 132298 | NM_015986 | Hs.7120 | cytokine receptor-like molecule 9 | 3.4 | 34 | 2 | 3 |
| 132316 | U28831 | Hs.44566 | KIAA1641 protein | 18.6 | 186 | 10 | 1.5 |
| 132325 | N37065 | Hs.44856 | hypothetical protein FLJ12116 | 5.5 | 323 | 59 | 10.5 |
| 132358 | NM_003542 | Hs.46423 | H4 histone family, member G | 3.3 | 979 | 298 | 2.2 |
| 132384 | AA312135 | Hs.46967 | HSPCO34 protein | 3.6 | 36 | 1 | 3.1 |
| 132388 | W32624 | Hs.278626 | Arg/Abl-interacting protein ArgBP2 | 5.9 | 186 | 32 | 3.7 |
| 132393 | AL135094 | Hs.47334 | hypothetical protein FLJ14495 | 4.2 | 159 | 38 | 7.1 |
| 132407 | BE613126 | Hs.47783 | B aggressive lymphoma gene | 4.6 | 46 | 1 | 4.3 |
| 132425 | N87549 | Hs.125287 | zinc finger protein ZNF140-like protein | 3.6 | 146 | 41 | 1.1 |
| 132440 | AB020699 | Hs.112751 | KIAA0892 protein | 3.3 | 33 | 4 | 2.9 |
| 132465 | AW169847 | Hs.49169 | KIAA1634 protein | 8.3 | 145 | 18 | 3.7 |
| 132522 | AB023164 | Hs.5070 | KIAA0947 protein | 4.6 | 46 | 1 | 4.4 |
| 132528 | T78736 | Hs.50758 | SMC4 (structural maintenance of chromoso | 9.3 | 93 | 1 | 8.4 |
| 132530 | AA306105 | Hs.50785 | SEC22, vesicle trafficking protein (S. c | 4.9 | 49 | 1 | 4.4 |
| 132543 | BE568452 | Hs.5101 | protein regulator of cytokinesis 1 | 11.8 | 201 | 17 | 19.1 |
| 132572 | AI929659 | Hs.237825 | signal recognition particle 72 kD | 3.8 | 38 | 1 | 3 |
| 132592 | AW803564 | Hs.288850 | Homo sapiens cDNA: FLJ22528 fis, clone H | 4.8 | 93 | 20 | 3.1 |
| 132602 | AW606927 | Hs.5306 | hypothetical protein DKFZp586F1122 simil | 6.1 | 61 | 2 | 5.9 |
| 132616 | BE262677 | Hs.283558 | hypothetical protein PRO1855 | 3.4 | 193 | 58 | 12.3 |
| 132617 | AF037335 | Hs.5338 | carbonic anhydrase XII | 14.2 | 390 | 28 | 22.5 |
| 132618 | AL050025 | Hs.279916 | hypothetical protein FLJ20151 | 3.3 | 909 | 274 | 3.2 |
| 132632 | AU076916 | Hs.5398 | guanine monphosphate synthetase | 5 | 50 | 1 | 4.1 |
| 132668 | AB018319 | Hs.5460 | KIAA0776 protein | 4.2 | 171 | 41 | 12.6 |
| 132742 | AA025480 | Hs.292812 | ESTs, Weakly similar to T33468 hypotheti | 6.5 | 65 | 1 | 5.6 |
| 132790 | AW242243 | Hs.168670 | peroxisomal famesylated protein | 3.7 | 37 | 1 | 2.2 |
| 132811 | U25435 | Hs.57419 | CCCTC-binding factor (zinc finger protei | 7 | 115 | 17 | 5.4 |
| 132852 | AL120050 | Hs.58220 | Homo sapiens cDNA: FLJ23005 fis, clone L | 3.3 | 61 | 19 | 5.1 |
| 132856 | NM_001448 | Hs.58367 | glypican 4 | 4.8 | 48 | 1 | 3.6 |
| 132880 | BE077155 | Hs.177537 | hypothetical protein DKFZp761B1514 | 12.6 | 126 | 8 | 9.9 |
| 132902 | AI936442 | Hs.59838 | hypothetical protein FLJ10808 | 11 | 187 | 17 | 10.4 |
| 132906 | BE613337 | Hs.234896 | geminin | 3.3 | 106 | 33 | 2.6 |
| 132914 | AL047045 | Hs.60293 | Homo sapiens clone 122482 unknown mRNA | 3.5 | 110 | 32 | 2.1 |
| 132968 | AF234532 | Hs.61638 | myosin X | 4.1 | 62 | 15 | 4.9 |
| 132977 | AA093322 | Hs.301404 | RNA binding motif protein 3 | 22.1 | 221 | 9 | 17.8 |
| 132990 | X77343 | Hs.334334 | transcription factor AP-2 alpha (activat | 12.7 | 311 | 25 | 2.4 |
| 132994 | AA112748 | Hs.279905 | clone HQ0310 PRO0310p1 | 3 | 380 | 127 | 5.5 |
| 133011 | NM_006379 | Hs.171921 | sema domain, immunoglobulin domain (Ig), | 7.3 | 271 | 37 | 2.3 |
| 133015 | AJ002744 | Hs.246315 | UDP-N-acetyl-alpha-D-galactosamine: polyp | 4.6 | 427 | 93 | 10.4 |
| 133070 | U92649 | Hs.64311 | a disintegrin and metalloproteinase doma | 3.6 | 36 | 1 | 3.1 |
| 133091 | AK001628 | Hs.64691 | KIAA0483 protein | 5.2 | 117 | 23 | 5 |
| 133192 | AA218564 | Hs.67052 | vacuolar protein sorting 26 (yeast homol | 3.1 | 359 | 118 | 2.5 |
| 133197 | AI275243 | Hs.180201 | hypothetical protein FLJ20671 | 5.1 | 58 | 12 | 5.7 |
| 133199 | AF231981 | Hs.250175 | homolog of yeast long chain polyunsatura | 3 | 816 | 275 | 3.9 |
| 133221 | W32474 | Hs.301746 | RAP2A, member of RAS oncogene family | 3.1 | 234 | 76 | 8.6 |
| 133240 | AK001489 | Hs.242894 | ADP-ribosylation factor-like 1 | 8.1 | 81 | 1 | 4.6 |
| 133271 | Z48633 | Hs.283742 | H. sapiens mRNA for retrotransposon | 12.4 | 124 | 6 | 10.8 |
| 133291 | BE297855 | Hs.69855 | NRAS-related gene | 3.3 | 33 | 1 | 2.9 |
| 133294 | AJ001388 | Hs.69997 | zinc finger protein 238 | 7.9 | 234 | 30 | 18.9 |
| 133350 | AI499220 | Hs.71573 | hypothetical protein FLJ10074 | 4.6 | 46 | 5 | 3.5 |
| 133362 | AK001519 | Hs.7194 | CGI-74 protein | 5 | 110 | 22 | 9.7 |
| 133370 | AF245505 | Hs.72157 | DKFZP564I1922 protein | 3.2 | 725 | 227 | 3.2 |
| 133407 | AF017987 | Hs.7306 | secreted frizzled-related protein 1 | 4.1 | 374 | 91 | 1.1 |
| 133422 | AB033061 | Hs.73287 | KIAA1235 protein | 4.3 | 43 | 1 | 3.9 |
| 133435 | AI929357 | Hs.323966 | Homo sapiens clone H63 unknown mRNA | 5.5 | 186 | 34 | 16.5 |
| 133479 | W01556 | Hs.238797 | ESTs, Moderately similar to I38022 hypot | 3.5 | 35 | 7 | 2.1 |
| 133493 | AW998046 | Hs.194369 | arginine-glutamic acid dipeptide (RE) re | 3.6 | 39 | 11 | 0.4 |
| 133504 | NM_004415 | Hs.74316 | desmoplakin (DPI, DPII) | 4.1 | 640 | 158 | 3 |
| 133517 | NM_000165 | Hs.74471 | gap junction protein, alpha 1, 43 kD (con | 3.2 | 351 | 111 | 5.2 |
| 133536 | W25797 | Hs.177486 | amyloid beta (A4) precursor protein (pro | 3.2 | 226 | 71 | 2.8 |
| 133578 | AU077050 | Hs.75066 | translin | 3.4 | 178 | 53 | 8.8 |
| 133633 | D21262 | Hs.75337 | nucleolar and coiled-body phosphprotein | 4.7 | 47 | 1 | 4 |
| 133640 | AW246428 | Hs.75355 | ubiquitin-conjugating enzyme E2N (homolo | 8.5 | 85 | 1 | 7.2 |
| 133669 | NM_006925 | Hs.166975 | splicing factor, arginine/serine-rich 5 | 3.6 | 36 | 1 | 0.4 |
| 133681 | AI352558 | Hs.75544 | tyrosine 3-monooxygenase/tryptophan 5-mo | 3.4 | 234 | 68 | 10.7 |
| 133746 | AW410035 | Hs.75862 | MAD (mothers against decapentaplegic, Dr | 9.3 | 93 | 1 | 7.8 |
| 133765 | M62194 | Hs.75929 | cadherin 11, type 2, OB-cadherin (osteob | 3.2 | 560 | 174 | 2.6 |
| 133780 | AA557660 | Hs.76152 | decorin | 5.4 | 144 | 27 | 13.3 |
| 133784 | BE622743 | Hs.301064 | arfaptin 1 | 4.7 | 47 | 1 | 4.1 |
| 133814 | NM_002462 | Hs.76391 | myxovirus (influenza) resistance 1, homo | 3.3 | 380 | 114 | 4.9 |
| 133829 | AW630088 | Hs.76550 | Homo sapiens mRNA; cDNA DKFZp564B1264 (f | 6.7 | 304 | 46 | 7.8 |
| 133845 | AA147026 | Hs.76704 | ESTs | 6.2 | 600 | 97 | 4.1 |
| 133913 | AU076964 | Hs.7753 | calumenin | 3.3 | 889 | 267 | 5 |
| 133968 | AA355986 | Hs.232068 | transcription factor 8 (represses interl | 3.7 | 91 | 25 | 2.6 |
| 133990 | R48316 | Hs.7822 | Homo sapiens mRNA; cDNA DKFZp564C1216 (f | 3.4 | 91 | 27 | 8.5 |
| 133999 | AA535244 | Hs.78305 | RAB2, member RAS oncogene family | 7.8 | 78 | 1 | 5.6 |
| 134032 | NM_005025 | Hs.78589 | serine (or cysteine) proteinase inhibito | 5.9 | 59 | 1 | 3.3 |
| 134064 | AF091622 | Hs.78893 | KIAA0244 protein | 5.8 | 58 | 1 | 4.9 |
| 134087 | U51166 | Hs.173824 | thymine-DNA glycosylase | 6.4 | 100 | 16 | 4.4 |
| 134089 | R51273 | Hs.79029 | ESTs | 5.1 | 51 | 9 | 3.8 |
| 134095 | NM_004354 | Hs.79069 | cyclin G2 | 5 | 50 | 1 | 3.2 |
| 134098 | BE513171 | Hs.79086 | mitochondrial ribosomal protein L3 | 4.8 | 246 | 51 | 3.9 |
| 134110 | U41060 | Hs.79136 | LIV-1 protein, estrogen regulated | 4.5 | 1472 | 330 | 2.1 |
| 134125 | NM_014781 | Hs.50421 | KIAA0203 gene product | 4.6 | 69 | 15 | 5.8 |
| 134246 | D28459 | Hs.80612 | ubiquitin-conjugating enzyme E2A (RAD6 h | 7 | 97 | 14 | 7.5 |
| 134257 | C05768 | Hs.8078 | Homo sapiens clone FBD3 Cri-du-chat crit | 3.4 | 34 | 5 | 2.6 |
| 134272 | X76040 | Hs.278614 | protease, serine, 15 | 3.6 | 36 | 1 | 2.8 |
| 134282 | R45621 | Hs.81057 | hypothetical protein MGC2718 | 6.7 | 67 | 9 | 5.7 |
| 134288 | AI022650 | Hs.8117 | erbb2-interacting protein ERBIN | 4.5 | 137 | 31 | 12 |
| 134321 | BE538082 | Hs.8172 | ESTs, Moderately similar to A46010 X-lin | 5.2 | 52 | 1 | 4.9 |
| 134326 | AW903838 | Hs.81800 | chondroitin sulfate proteoglycan 2 (vers | 8.6 | 568 | 66 | 22.4 |
| 134328 | AW959281 | Hs.8184 | ESTs | 4.8 | 53 | 11 | 3.7 |
| 134348 | AW291946 | Hs.82065 | interleukin 6 signal transducer (gp130, | 7.1 | 71 | 4 | 6.4 |
| 134359 | NM_001982 | Hs.199067 | v-erb-b2 avian erythroblastic leukemia v | 3 | 68 | 23 | 2.8 |
| 134367 | AA339449 | Hs.82285 | phosphoribosylglycinamide formyltransfer | 4.4 | 44 | 1 | 4.1 |
| 134374 | N22687 | Hs.8236 | ESTs | 13.3 | 445 | 34 | 6 |
| 134380 | AU077143 | Hs.179565 | minichromosome maintenance deficient (S. | 4.5 | 45 | 2 | 3.4 |
| 134395 | AA456539 | Hs.8262 | lysosomal | 6 | 60 | 5 | 5.9 |
| 134401 | AI916662 | Hs.211577 | kinectin 1 (kinesin receptor) | 4.1 | 301 | 73 | 6.1 |
| 134405 | AW067903 | Hs.82772 | collagen, type XI, alpha 1 | 4.6 | 1216 | 267 | 4.4 |
| 134415 | AI750762 | Hs.82911 | protein tyrosine phosphatase type IVA, m | 4.9 | 163 | 34 | 15.1 |
| 134417 | NM_006416 | Hs.82921 | solute carrier family 35 (CMP-sialic aci | 4.9 | 49 | 3 | 3.8 |
| 134419 | W95642 | Hs.82961 | trefoil factor 3 (intestinal) | 3.2 | 1872 | 592 | 3.3 |
| 134421 | AU077196 | Hs.82985 | collagen, type V, alpha 2 | 6.3 | 1075 | 171 | 3.8 |
| 134436 | U29344 | Hs.83190 | fatty acid synthase | 3.3 | 710 | 217 | 2 |
| 134485 | X82153 | Hs.83942 | cathepsin K (pycnodysostosis) | 34.3 | 411 | 12 | 5.1 |
| 134487 | AF061739 | Hs.83954 | protein associated with PRK1 | 4.8 | 153 | 32 | 4.3 |
| 134495 | D63477 | Hs.84087 | KIAA0143 protein | 3.1 | 147 | 48 | 12.7 |
| 134520 | BE091005 | Hs.74861 | activated RNA polymerase II transcriptio | 3.3 | 33 | 1 | 2 |
| 134542 | M14156 | Hs.85112 | insulin-like growth factor 1 (somatomedi | 4.2 | 42 | 5 | 2.6 |
| 134570 | U66615 | Hs.172280 | SWI/SNF related, matrix associated, acti | 3.9 | 39 | 1 | 2.5 |
| 134590 | AW903849 | Hs.173840 | HUEL (C4orf1)-interacting protein | 3.7 | 41 | 11 | 0.6 |
| 134604 | NM_002884 | Hs.865 | RAP1A, member of RAS oncogene family | 5.2 | 52 | 1 | 3 |
| 134612 | AW068223 | Hs.171581 | ubiquitin C-terminal hydrolase UCH37 | 4.9 | 49 | 1 | 3.7 |
| 134643 | AW299723 | Hs.87223 | bone morphogenetic protein receptor, typ | 5.2 | 52 | 5 | 3.5 |
| 134654 | AK001741 | Hs.8739 | hypothetical protein FLJ10879 | 6.4 | 64 | 1 | 5.1 |
| 134656 | AI750878 | Hs.87409 | thrombospondin 1 | 12.6 | 126 | 1 | 10.8 |
| 134672 | AF271212 | Hs.322901 | disrupter of silencing 10 | 5.4 | 81 | 15 | 2.6 |
| 134700 | AK000606 | Hs.8868 | golgi SNAP receptor complex member 1 | 3.4 | 179 | 52 | 1.5 |
| 134711 | X04011 | Hs.88974 | cytochrome b-245, beta polypeptide (chro | 3.2 | 143 | 45 | 13.9 |
| 134722 | AF129536 | Hs.284226 | F-box only protein 6 | 7 | 70 | 6 | 6 |
| 134856 | BE281128 | Hs.9030 | TONDU | 3.1 | 31 | 1 | 2.3 |
| 134880 | AI879195 | Hs.90606 | 15 kDa selenoprotein | 5.7 | 57 | 1 | 5 |
| 134917 | X87241 | Hs.166994 | FAT tumor suppressor (Drosophila) homolo | 3.2 | 153 | 48 | 4.7 |
| 134921 | AL137491 | Hs.125511 | Homo sapiens mRNA; cDNA DKFZp434P1530 (f | 4 | 452 | 114 | 2 |
| 134982 | AK002085 | Hs.92308 | Homo sapiens cDNA FLJ11223 fis, clone PL | 5.1 | 150 | 30 | 7.2 |
| 134989 | AW968058 | Hs.92381 | nudix (nucleoside diphosphate linked moi | 8.2 | 114 | 14 | 9.9 |
| 135029 | H58818 | Hs.187579 | hydroxysteroid (17-beta) dehydrogenase 7 | 11.5 | 115 | 1 | 10 |
| 135035 | AL034344 | Hs.284186 | forkhead box C1 | 5.4 | 259 | 48 | 1.4 |
| 135051 | AI272141 | Hs.83484 | SRY (sex determining region Y)-box 4 | 3.3 | 1296 | 394 | 2.2 |
| 135062 | AK000967 | Hs.93872 | KIAA1682 protein | 3.8 | 240 | 64 | 3.2 |
| 135073 | W55956 | Hs.94030 | Homo sapiens mRNA; cDNA DKFZp586E1624 (f | 8.1 | 101 | 13 | 7.9 |
| 135098 | AW274526 | Hs.277721 | ovarian carcinoma antigen CA125 | 3.3 | 33 | 1 | 2.6 |
| 135117 | W52493 | Hs.94694 | Homo sapiens cDNA FLJ10561 fis, clone NT | 5.3 | 53 | 1 | 4.1 |
| 135144 | NM_016255 | Hs.95260 | Autosomal Highly Conserved Protein | 7.4 | 74 | 5 | 2.4 |
| 135154 | AK001835 | Hs.267812 | sorting nexin 4 | 6.6 | 69 | 11 | 6.3 |
| 135155 | AI207958 | Hs.166556 | Homo sapiens, Similar to TEA domain fami | 6.1 | 61 | 1 | 5.1 |
| 135172 | AB028956 | Hs.12144 | KIAA1033 protein | 3.4 | 88 | 26 | 1.4 |
| 135242 | AI583187 | Hs.9700 | cyclin E1 | 3.1 | 31 | 1 | 2.3 |
| 135243 | BE463721 | Hs.97101 | putative G protein-coupled receptor | 3.4 | 169 | 50 | 9.1 |
| 135269 | NM_003403 | Hs.97496 | YY1 transcription factor | 3.4 | 475 | 142 | 2.5 |
| 135356 | BE312948 | Hs.18104 | hypothetical protein FLJ11274 | 3.1 | 31 | 10 | 1.7 |
| 135357 | AI565004 | Hs.79572 | cathepsin D (lysosomal aspartyl protease | 4.7 | 710 | 151 | 2.5 |
| 135389 | U05237 | Hs.99872 | fetal Alzheimer antigen | 20.6 | 206 | 4 | 19.1 |
| 135397 | L14922 | Hs.166563 | replication factor C (activator 1) 1 (14 | 3.2 | 32 | 1 | 2.4 |
| 135400 | X78592 | Hs.99915 | androgen receptor (dihydrotestosterone r | 3.2 | 117 | 37 | 9.4 |
| | AI471525 | Hs.247486 | ESTs | 3.8 | 58 | 16 | 5.5 |
| | X70683 | Hs.93668 | ESTs | 1.8 | 1047 | 596 | 1.6 |
| | L14922 | Hs.82128 | 5T4 oncofetal trophoblast glycoprotein | 5 | 285 | 58 | 1.2 |
| | M23263 | Hs.904 | amylo-1;6-glucosidase;4-alpha-glucanotransferas | 3.1 | 31 | 1 | 2.6 |
| | AI267886 | Hs.148027 | polymerase (RNA) II (DNA directed) polypeptide B | 7.8 | 137 | 18 | 11.9 |
| | AA044840 | Hs.241676 | stromal cell-derived factor 1 | 4.7 | 114 | 25 | 0.9 |
| | N90960 | Hs.227459 | ESTs; Moderately similar to !!!! ALU SUBFAMILY | 4.7 | 151 | 32 | 9.3 |
| | AA873285 | Hs.137947 | ESTs | 4.7 | 47 | 3 | 4.4 |
| | T56679 | Hs.865 | RAP1A; member of RAS oncogene family | 4 | 40 | 1 | 3.4 |
| | AA305536 | | “EST176522 Colon carcinoma (Caco-2) cell line II | 3.6 | 121 | 34 | 11.8 |
| | AI369384 | | arylsulfatase D | 3.5 | 113 | 33 | 1.7 |
| | AA219081 | Hs.242396 | ESTs; Moderately similar to !!!! ALU SUBFAMILY] | 3.4 | 107 | 32 | 9.9 |

Table 10 depicts a preferred group of genes upregulated in tumor tissue compared to normal breast tissue.
Pkey: Unique Eos probeset identifier number
ExAccn: Exemplar Accession number, Genbank accession number
UnigeneID: Unigene number
Unigene Title: Unigene gene title
R1: Ratio of tumor to normal body tissue
R2: Ratio of 90^{th} percentile tumor to body
R3: Ratio of 75^{th} percentile body to tumor
R4: Ratio of tumor to normal breast tissue

TABLE 10A
Table 10 A shows the accession numbers for those pkeys lacking unigeneID's for Table 10.
For each probeset, we have listed the gene cluster number from which the oligonucleotides
were designed. Gene clusters were compiled using sequences derived from Genbank ESTs
and mRNAs. These sequences were clustered based on sequence similarity using Clustering
and Alignment Tools (DoubleTwist, Oakland California). The Genbank accession numbers
for sequences comprising each cluster are listed in the “Accession” column.

| | |
|---|---|
| Pkey: | Unique Eos probeset identifier number |
| CAT number: | Gene cluster number |
| Accession: | Genbank accession numbers |

| Pkey | CAT number | Accession |
|---|---|---|
| 123619 | 371681_1 | AA602964 AA609200 |
| 104602 | 524482_2 | H47610 R86920 |
| 121581 | 283769_1 | AA416568 AA442889 AA417233 AA442223 |
| 123523 | genbank_AA608588 | AA608588 |
| 100821 | tigr_HT4306 | M26460 U09116 |
| 125091 | genbank_T91518 | T91518 |
| 125150 | NOT_FOUND_entrez_W38240 | W38240 |
| 118475 | genbank_N66845 | N66845 |
| 104787 | genbank_AA027317 | AA027317 |
| 106055 | genbank_AA417034 | AA417034 |
| 113702 | genbank_T97307 | T97307 |
| 101046 | entrez_K01160 | K01160 |
| 101447 | entrez_M21305 | M21305 |
| 101624 | entrez_M55998 | M55998 |
| 124677 | genbank_R01073 | R01073 |
| 110581 | genbank_H61560 | H61560 |
| 119023 | genbank_N98488 | N98488 |
| 110775 | genbank_N22414 | N22414 |
| 112092 | genbank_R44538 | R44538 |
| 112253 | genbank_R51818 | R51818 |
| 107014 | genbank_AA598820 | AA598820 |
| 114988 | genbank_AA251089 | AA251089 |

TABLE 11
FIG. 11 from BRCA 001-3 PCT

| Pkey | ExAccn | UnigeneID | Unigene Title | R1 | R2 | R3 | R4 |
|---|---|---|---|---|---|---|---|
| 100131 | D12485 | Hs.11951 | ectonucleotide pyrophosphatase/phosphodi | 13.2 | 244 | 19 | 9.9 |
| 100147 | D13666 | Hs.136348 | osteoblast specific factor 2 (fasciclin | 15.7 | 1030 | 66 | 5 |
| 100522 | X51501 | Hs.99949 | prolactin-induced protein | 22.7 | 760 | 34 | 1.4 |
| 100666 | L05424 | Hs.169610 | CD44 antigen (homing function and Indian | 8.5 | 85 | 1 | 3.2 |
| 101104 | AW862258 | HS.169266 | neuropeptide Y receptor Y1 | 15.3 | 153 | 1 | 14.1 |
| 101478 | NM_002890 | Hs.758 | RAS p21 protein activator (GTPase activa | 9.6 | 96 | 1 | 8.5 |
| 101724 | L11690 | Hs.620 | bullous pemphigoid antigen 1 (230/240 kD) | 9.4 | 94 | 1 | 0.3 |
| 101754 | S70114 | Hs.239489 | TIA1 cytotoxic granule-associated RNA-bi | 8.9 | 89 | 5 | 8 |
| 101888 | AL049610 | Hs.95243 | transcription elongation factor A (SII)- | 7.3 | 73 | 1 | 5.3 |
| 102165 | BE313280 | Hs.159627 | death associated protein 3 | 9.3 | 93 | 5 | 8 |
| 102304 | AF015224 | Hs.46452 | mammaglobin 1 | 8.5 | 2058 | 243 | 1.4 |
| 102348 | U37519 | Hs.87539 | aldehyde dehydrogenase 3 family, member | 6.4 | 428 | 67 | 2.3 |
| 102457 | NM_001394 | Hs.2359 | dual specificity phosphatase 4 | 20.2 | 202 | 5 | 1.3 |
| 102567 | U63830 | Hs.146847 | TRAF family member-associated NFKB activ | 8.2 | 82 | 1 | 6.8 |
| 102823 | D85390 | Hs.5057 | carboxypeptidase D | 5.6 | 56 | 1 | 5.3 |
| 103557 | AL133415 | Hs.297753 | vimentin | 7.5 | 136 | 18 | 3.4 |
| 103613 | NM_000346 | Hs.2316 | SRY (sex determining region Y)-box 9 (ca | 7.3 | 73 | 1 | 5.2 |
| 104115 | AF183810 | Hs.26102 | opposite strand to trichorhinophalangeal | 29 | 290 | 1 | 26.8 |
| 104667 | AI239923 | Hs.30098 | ESTs | 14.9 | 149 | 1 | 6.4 |
| 104804 | AI858702 | Hs.31803 | ESTs, Weakly similar to N-WASP [H. sapien | 7.7 | 77 | 1 | 5.1 |
| 104807 | AI139058 | Hs.125790 | leucine-rich repeat-containing 2 | 7 | 70 | 1 | 6.5 |
| 104896 | AW015318 | Hs.23165 | ESTs | 7.4 | 74 | 1 | 6 |
| 104943 | AF072873 | Hs.114218 | frizzled (Drosophila) homolog 6 | 16.2 | 162 | 1 | 4.2 |
| 105038 | AW503733 | Hs.9414 | KIAA1488 protein | 5.5 | 55 | 1 | 5.2 |
| 105329 | AA234561 | Hs.22862 | ESTs | 2.8 | 131 | 47 | 3.9 |
| 105500 | AW602166 | Hs.222399 | CEGP1 protein | 25.4 | 508 | 20 | 3 |
| 105516 | AK001269 | Hs.30738 | hypothetical protein FLJ10407 | 8.3 | 83 | 3 | 1.8 |
| 105730 | AW377314 | Hs.5364 | DKFZP564I052 protein | 6.9 | 69 | 1 | 4.4 |
| 106012 | AI240665 | Hs.8895 | ESTs | 21.2 | 212 | 6 | 17.4 |
| 106095 | AF115402 | Hs.11713 | E74-like factor 5 (ets domain transcript | 26.3 | 356 | 14 | 1 |
| 106155 | AA425414 | Hs.33287 | nuclear factor I/B | 9.9 | 483 | 49 | 1.8 |
| 107102 | AB037765 | Hs.30652 | KIAA1344 protein | 6.3 | 63 | 1 | 5.4 |
| 107136 | AV661958 | Hs.8207 | GK001 protein | 2.5 | 392 | 155 | 4.3 |
| 107151 | AW378065 | Hs.8687 | ESTs | 15.6 | 156 | 7 | 10.8 |
| 107922 | BE153855 | Hs.61460 | Ig superfamily receptor LNIR | 9 | 90 | 1 | 5.5 |
| 108339 | AW151340 | Hs.51615 | ESTs, Weakly similar to ALU7_HUMAN ALU S | 18.7 | 187 | 1 | 17 |
| 109112 | AW419196 | Hs.257924 | hypothetical protein FLJ13782 | 4.1 | 334 | 82 | 3.4 |
| 109292 | AW975746 | Hs.188662 | KIAA1702 protein | 7.1 | 71 | 1 | 6.5 |
| 109415 | U80736 | Hs.110826 | trinucleotide repeat containing 9 | 12.3 | 123 | 1 | 11.3 |
| 109912 | AW390822 | Hs.301528 | L-kynurenine/alpha-aminoadipate aminotra | 14.2 | 142 | 1 | 9.5 |
| 110009 | BE075297 | Hs.6614 | ESTs, Weakly similar to A43932 mucin 2 p | 6.3 | 693 | 110 | 7.2 |
| 110915 | BE092285 | Hs.29724 | hypothetical protein FLJ13187 | 20.9 | 209 | 1 | 19.5 |
| 111164 | N46180 | Hs.122489 | Homo sapiens cDNA FLJ13289 fis, clone OV | 7.7 | 77 | 1 | 5 |
| 111179 | AK000136 | Hs.10760 | asporin (LRR class 1) | 25.1 | 288 | 12 | 6.7 |
| 111190 | AK002055 | Hs.151046 | hypothetical protein FLJ11193 | 6.3 | 63 | 1 | 5.8 |
| 111223 | AA852773 | Hs.334838 | KIAA1866 protein | 3.6 | 402 | 112 | 4.9 |
| 111357 | BE314949 | Hs.87128 | hypothetical protein FLJ23309 | 3.8 | 425 | 111 | 4 |
| 112244 | AB029000 | Hs.70823 | KIAA1077 protein | 5.7 | 567 | 100 | 6.7 |
| 113047 | AI571940 | Hs.7549 | ESTs | 9.6 | 124 | 13 | 9 |
| 113702 | T97307 | | gb: ye53h05.s1 Soares fetal liver spleen | 12.3 | 129 | 11 | 11.7 |
| 114124 | W57554 | Hs.125019 | lymphoid nuclear protein (LAF-4) mRNA | 24.2 | 242 | 10 | 5.6 |
| 114138 | AW384793 | Hs.15740 | Homo sapiens mRNA; cDNA DKFZp434E033 (fr | 6.7 | 67 | 1 | 6.3 |
| 114768 | AF212848 | Hs.182339 | ets homologous factor | 13.7 | 137 | 1 | 8.9 |
| 114860 | AL157545 | Hs.42179 | bromodomain and PHD finger containing, 3 | 9.1 | 91 | 1 | 7.6 |
| 114965 | AI733881 | Hs.72472 | BMP-R1B | 35.9 | 359 | 10 | 29.7 |
| 114988 | AA251089 | | gb: zs04f05.s1 NCI_CGAP.GCB1 Homo sapiens | 11.5 | 115 | 1 | 6.9 |
| 115206 | AW183695 | Hs.186572 | ESTs | 5.8 | 58 | 1 | 5 |
| 115719 | AW992405 | Hs.59622 | Homo sapiens, clone IMAGE: 3507281, mRNA, | 7.6 | 144 | 19 | 13.9 |
| 115844 | AI373062 | Hs.332938 | hypothetical protein MGC5370 | 6.2 | 62 | 1 | 5.4 |
| 116470 | AI272141 | Hs.83484 | SRY (sex determining region Y)-box 4 | 1.8 | 1047 | 596 | 1.6 |
| 116786 | H25836 | Hs.301527 | ESTs, Moderately similar to unknown [H. s | 22.8 | 228 | 9 | 12.4 |
| 117280 | M18217 | Hs.172129 | Homo sapiens cDNA: FLJ21409 fis, clone C | 3.9 | 322 | 83 | 4.4 |
| 117412 | N32536 | Hs.42645 | solute carrier family 16 (monocarboxylic | 17.4 | 174 | 9 | 6.9 |
| 118472 | AL157545 | Hs.42179 | bromodomain and PHD finger containing, 3 | 14.5 | 145 | 1 | 2.4 |
| 119271 | AI061118 | Hs.65328 | Fanconi anemia, complementation group F | 8.2 | 82 | 1 | 6.4 |
| 119771 | AI905687 | Hs.2533 | EST | 3.5 | 2073 | 595 | 2.1 |
| 120562 | BE244580 | Hs.302267 | hypothetical protein FLJ10330 | 8.5 | 127 | 15 | 1.6 |
| 121463 | AK000282 | Hs.239681 | hypothetical protein FLJ20275 | 10.3 | 103 | 1 | 9.3 |
| 121723 | AA243499 | Hs.104800 | hypothetical protein FLJ10134 | 2.9 | 214 | 74 | 3.7 |
| 122963 | AA478446 | Hs.69559 | KIAA1096 protein | 7.2 | 72 | 1 | 5.7 |
| 123137 | AI073913 | Hs.100686 | ESTs, Weakly similar to JE0350 Anterior | 9.9 | 351 | 36 | 13.9 |
| 123619 | AA602964 | | gb: no97c02.s1 NCI_CGAP_Pr2 Homo sapiens | 8.5 | 85 | 1 | 4.3 |
| 123709 | AA706910 | Hs.112742 | ESTs | 3.9 | 60 | 16 | 4.8 |
| 124006 | AI147155 | Hs.270016 | ESTs | 5.8 | 321 | 55 | 17 |
| 124059 | BE387335 | Hs.283713 | ESTs, Weakly similar to S64054 hypotheti | 10.4 | 880 | 85 | 5.3 |
| 124308 | AA249027 | Hs.241507 | ribosomal protein S6 | 10.5 | 105 | 1 | 9.9 |
| 125279 | AW401809 | Hs.4779 | KIAA1150 protein | 13.1 | 131 | 1 | 5.1 |
| 125617 | AA287921 | Hs.164950 | ESTs | 6.7 | 67 | 1 | 6 |
| 127439 | D60237 | Hs.14368 | SH3 domain binding glutamic acid-rich pr | 30.6 | 306 | 4 | 26.5 |
| 128305 | AI954968 | Hs.279009 | matrix Gla protein | 7.5 | 75 | 1 | 6.5 |
| 128482 | AI694143 | Hs.296251 | programmed cell death 4 | 7.2 | 72 | 1 | 5.8 |
| 128790 | AF026692 | Hs.105700 | secreted frizzled-related protein 4 | 17.4 | 409 | 24 | 7.8 |
| 128925 | R67419 | Hs.21851 | Homo sapiens cDNA FLJ12900 fis, clone NT | 7.1 | 392 | 56 | 3.6 |
| 129017 | AA115333 | Hs.107968 | ESTs | 8.2 | 82 | 1 | 7.4 |
| 129229 | AF013758 | Hs.109643 | polyadenylate binding protein-interactin | 7.1 | 71 | 1 | 6.2 |
| 129337 | NM_014918 | Hs.110488 | KIAA0990 protein | 9.5 | 95 | 1 | 8.5 |
| 129366 | BE220806 | Hs.184697 | Homo sapiens clone 23785 mRNA sequence | 7.1 | 150 | 21 | 14.5 |
| 129821 | AB028945 | Hs.12696 | cortactin SH3 domain-binding protein | 11.4 | 114 | 1 | 10 |
| 130036 | BE061916 | Hs.125849 | chromosome 8 open reading frame 2 | 6.7 | 67 | 1 | 5.7 |
| 130057 | AF027153 | Hs.324787 | solute carrier family 5 (inositol transp | 1 | 1 | 1 | 1 |
| 130095 | AK001635 | Hs.14838 | hypothetical protein FLJ10773 | 14.6 | 219 | 15 | 7.6 |
| 130343 | AB040914 | Hs.278628 | KIAA1481 protein | 13.2 | 331 | 25 | 12.4 |
| 130385 | AW067800 | Hs.155223 | stanniocalcin 2 | 72.2 | 722 | 1 | 1.9 |
| 130407 | BE385099 | Hs.334727 | hypothetical protein MGC3017 | 6.5 | 65 | 4 | 5.3 |
| 130441 | U63630 | Hs.155637 | protein kinase, DNA-activated, catalytic | 6.1 | 61 | 1 | 5.7 |
| 130455 | D90041 | Hs.155956 | N-acetyltransferase 1 (arylamine N-acety | 10.8 | 706 | 66 | 9.2 |
| 130604 | AA383256 | Hs.1657 | estrogen receptor 1 | 32.2 | 322 | 1 | 4.7 |
| 130617 | M90516 | Hs.1674 | glutamine-fructose-6-phosphate transamin | 10 | 100 | 1 | 7.6 |
| 130712 | AJ271881 | Hs.279762 | bromodomain-containing 7 | 17.5 | 175 | 2 | 12.8 |
| 131148 | AW953575 | Hs.303125 | p53-induced protein PIGPC1 | 3.8 | 585 | 153 | 3.7 |
| 131388 | NM_014810 | Hs.92200 | KIAA0480 gene product | 7.6 | 76 | 1 | 5 |
| 131564 | T93500 | Hs.28792 | Homo sapiens cDNA FLJ11041 fis, clone PL | 4.7 | 381 | 81 | 6.4 |
| 131742 | AA961420 | Hs.31433 | ESTs | 11.7 | 117 | 1 | 10.1 |
| 131877 | J04088 | Hs.156346 | topoisomerase (DNA) II alpha (170 kD) | 6.8 | 68 | 1 | 5.6 |
| 131985 | AA503020 | Hs.36563 | hypothetical protein FLJ22418 | 40.2 | 402 | 1 | 4 |
| 132316 | U28831 | Hs.44566 | KIAA1641 protein | 18.6 | 186 | 10 | 1.5 |
| 132528 | T78736 | Hs.50758 | SMC4 (structural maintenance of chromoso | 9.3 | 93 | 1 | 8.4 |
| 132742 | AA025480 | Hs.292812 | ESTs, Weakly similar to T33468 hypotheti | 6.5 | 65 | 1 | 5.6 |
| 132990 | X77343 | Hs.334334 | transcription factor AP-2 alpha (activat | 12.7 | 311 | 25 | 2.4 |
| 133015 | AJ002744 | Hs.246315 | UDP-N-acetyl-alpha-D-galactosamine: polyp | 4.6 | 427 | 93 | 10.4 |
| 133199 | AF231981 | Hs.250175 | homolog of yeast long chain polyunsatura | 3 | 816 | 275 | 3.9 |
| 133240 | AK001489 | Hs.242894 | ADP-ribosylation factor-like 1 | 8.1 | 81 | 1 | 4.6 |
| 133271 | Z48633 | Hs.283742 | H. sapiens mRNA for retrotransposon | 12.4 | 124 | 6 | 10.8 |
| 133640 | AW246428 | Hs.75355 | ubiquitin-conjugating enzyme E2N (homolo | 8.5 | 85 | 1 | 7.2 |
| 133746 | AW410035 | Hs.75862 | MAD (mothers against decapentaplegic, Dr | 9.3 | 93 | 1 | 7.8 |
| 133999 | AA535244 | Hs.78305 | RAB2, member RAS oncogene family | 7.8 | 78 | 1 | 5.6 |
| 134110 | U41060 | Hs.79136 | LIV-1 protein, estrogen regulated | 4.5 | 1472 | 330 | 2.1 |
| 134485 | X82153 | Hs.83942 | cathepsin K (pycnodysostosis) | 34.3 | 411 | 12 | 5.1 |
| 134654 | AK001741 | Hs.8739 | hypothetical protein FLJ10879 | 6.4 | 64 | 1 | 5.1 |
| 134880 | AI879195 | Hs.90606 | 15 kDa selenoprotein | 5.7 | 57 | 1 | 5 |
| 135029 | H58818 | Hs.187579 | hydroxysteroid (17-beta) dehydrogenase 7 | 11.5 | 115 | 1 | 10 |
| 135389 | U05237 | Hs.99872 | fetal Alzheimer antigen | 20.6 | 206 | 4 | 19.1 |
| 128305 | AI954968 | Hs.279009 | matrix Gla protein | 9.4 | 94 | 3 | 5.3 |

Table 11 depicts a preferred group of genes upregulated in tumor tissue compared to normal breast tissue.
Pkey: Unique Eos probeset identifier number
ExAccn: Exemplar Accession number, Genbank accession number
UnigeneID: Unigene number
Unigene Title: Unigene gene title
R1: Ratio of tumor to normal body tissue
R2: Ratio of 90^{th} percentile tumor to normal body
R3: Ratio of 75^{th} percentile normal body to tumor
R4: Ratio of tumor to normal breast tissue

TABLE 11A
Table 11A shows the accession numbers for those pkeys lacking
unigeneID's for Table 11. For each probeset, we have listed the gene
cluster number from which the oligonucleotides were designed. Gene
clusters were compiled using sequences derived from Genbank ESTs
and mRNAs. These sequences were clustered based on sequence similarity
using Clustering and Alignment Tools (DoubleTwist, Oakland California).
The Genbank accession numbers for sequences comprising each cluster are
listed in the “Accession” column.

| | |
|---|---|
| Pkey: | Unique Eos probeset identifier number |
| CAT number: | Gene cluster number |
| Accession: | Genbank accession numbers |

| Pkey | CAT number | Accession |
|---|---|---|
| 123619 | 371681_1 | AA602964 AA609200 |
| 113702 | genbank_T97307 | T97307 |
| 114988 | genbank_AA251089 | AA251089 |

TABLE 12
FIG. 12 from BRCA 001-3 PCT

| Pkey | ExAccn | UnigeneID | Unigene Title | R1 | R2 | R3 | R4 |
|---|---|---|---|---|---|---|---|
| 100131 | D12485 | Hs.11951 | phosphodiesterase I (PC-1) | 13.2 | 244 | 19 | 9.9 |
| 105500 | AW602166 | Hs.222399 | ESTs | 25.4 | 508 | 20 | 3 |
| 112244 | AB029000 | Hs.70823 | KIAA1077 protein | 5.7 | 567 | 100 | 6.7 |
| 114124 | W57554 | Hs.125019 | ESTs | 24.2 | 242 | 10 | 5.6 |
| 119771 | AI905687 | Hs.2533 | ESTs | 3.5 | 2073 | 595 | 2.1 |
| 121723 | AA243499 | Hs.104800 | ESTs | 2.9 | 214 | 74 | 3.7 |
| 128790 | AF026692 | Hs.105700 | secreted frizzled-related protein 4 | 17.4 | 409 | 24 | 7.8 |
| 131148 | AW953575 | Hs.303125 | ESTs | 3.8 | 585 | 153 | 3.7 |
| 131985 | AA503020 | Hs.36563 | ESTs | 40.2 | 402 | 1 | 4 |
| 133199 | AF231981 | Hs.250175 | Homo sapiens clone 23904 mRNA sequence | 3 | 816 | 275 | 3.9 |

Table 12 depicts a preferred group of genes upregulated in tumor tissue compared to normal breast tissue.
Pkey: Unique Eos probeset identifier number
ExAccn: Exemplar Accession number, Genbank accession number
UnigeneID: Unigene number
Unigene Title: Unigene gene title
R1: Ratio of tumor to normal body tissue
R2: Ratio of 90^{th} percentile tumor to body
R3: Ratio of 75^{th} percentile body to tumor
R4: Ratio of tumor to normal breast tissue

TABLE 13
Table 1 from BRCA 001-5 US
Table 13 depicts a preferred group of genes upregulated in breast cancer cells.

| Pkey | ExAccn | UniGene ID | Unigene Title | R1 |
|---|---|---|---|---|
| 100038 | M97935 | | control | 16.7 |
| 100039 | M97935 | | control | 6.3 |
| 100040 | M97935 | | control | 8.3 |
| 100041 | M97935 | | control | 14.8 |
| 100082 | AB003103 | Hs.4295 | proteasome (prosome; macropain) 26S sub | 7.5 |
| 100091 | AF000177 | Hs.111783 | Lsm1 protein | 4.9 |
| 100100 | AF006084 | Hs.11538 | actin related protein 2/3 complex; subunit | 4.7 |
| 100103 | AF007875 | Hs.5085 | dolichyl-phosphate mannosyltransferase p | 13.4 |
| 100114 | D00596 | Hs.82962 | thymidylate synthetase | 15.9 |
| 100121 | D10495 | Hs.155342 | protein kinase C; delta | 4.6 |
| 100123 | D10523 | Hs.168669 | oxoglutarate dehydrogenase (lipoamide) | 7.5 |
| 100128 | D11094 | Hs.61153 | proteasome (prosome; macropain) 26S sub | 4.4 |
| 100131 | D12485 | Hs.11951 | phosphodiesterase I/nucleotide pyrophosp | 8.7 |
| 100137 | D13627 | Hs.15071 | chaperonin containing TCP1; subunit 8 (t | 9.5 |
| 100144 | D13643 | Hs.75616 | Human mRNA for KIAA0018 gene; comp | 6 |
| 100147 | D13666 | Hs.136348 | osteoblast specific factor 2 (fasciclin I-like | 8.5 |
| 100154 | D14657 | Hs.81892 | KIAA0101 gene product | 10.5 |
| 100164 | D14812 | Hs.173714 | MORF-related gene X | 4.6 |
| 100169 | D14878 | Hs.82043 | D123 gene product | 7.9 |
| 100190 | D21090 | Hs.178658 | RAD23 (S. cerevisiae) homolog B | 5.6 |
| 100203 | D25538 | Hs.172199 | adenylate cyclase 7 | 9.9 |
| 100209 | D26308 | Hs.76289 | biliverdin reductase B (flavin reductase (N | 4.9 |
| 100215 | D26598 | Hs.82793 | proteasome (prosome; macropain) subunit | 14.2 |
| 100216 | D26599 | Hs.1390 | proteasome (prosome; macropain) subunit | 11.3 |
| 100219 | D28137 | Hs.118110 | bone marrow stromal cell antigen 2 | 5.7 |
| 100227 | D28915 | Hs.82316 | interferon-induced; hepatitis C-associated | 5.7 |
| 100248 | D31888 | Hs.78398 | KIAA0071 protein | 7.4 |
| 100287 | D43950 | Hs.1600 | chaperonin containing TCP1; subunit 5 (e | 5.6 |
| 100294 | D49396 | Hs.75454 | antioxidant protein 1 | 12.9 |
| 100307 | D50525 | Hs.699 | hypothetical protein | 8.4 |
| 100335 | D63391 | Hs.6793 | platelet-activating factor acetylhydrolase; | 6.8 |
| 100340 | D63487 | Hs.82563 | KIAA0153 protein | 4.4 |
| 100355 | D78129 | Hs.71465 | Homo sapiens mRNA for squalene epoxid | 12.6 |
| 100363 | D78514 | Hs.78563 | ubiquitin-conjugating enzyme E2G 1 (hom | 4.6 |
| 100368 | D79987 | Hs.153479 | extra spindle poles; S. cerevisiae; homolo | 6.5 |
| 100372 | D79997 | Hs.184339 | KIAA0175 gene product | 8.4 |
| 100375 | D80004 | Hs.75909 | KIAA0182 protein | 4.5 |
| 100379 | D82060 | Hs.278721 | Ke4 gene; mouse; human homolog of | 8.1 |
| 100387 | D83777 | Hs.75137 | KIAA0193 gene product | 10.7 |
| 100393 | D84145 | Hs.39913 | novel RGD-containing protein | 7.2 |
| 100398 | D84557 | Hs.155462 | minichromosome maintenance deficient (m | 7.2 |
| 100405 | D86425 | Hs.82733 | nidogen 2 | 5.4 |
| 100406 | D86479 | Hs.118397 | AE-binding protein 1 | 4.3 |
| 100409 | D86957 | Hs.80712 | KIAA0202 protein | 11.9 |
| 100421 | D86985 | Hs.79276 | Human mRNA for KIAA0232 gene; comp | 9.7 |
| 100446 | D87464 | Hs.10037 | KIAA0274 gene product | 6.4 |
| 100447 | D87465 | Hs.74583 | KIAA0275 gene product | 10 |
| 100448 | D87469 | Hs.57652 | EGF-like-domain; multiple 2 | 6.2 |
| 100467 | D89052 | Hs.7476 | ATPase; H+ transporting; lysosomal (vacu | 7.5 |
| 100468 | D89289 | Hs.118722 | fucosyltransferase 8 (alpha (1;6) fucosyltr | 5 |
| 100486 | HT1112 | Hs.10842 | Ras-Like Protein Tc4 | 16.9 |
| 100497 | HT1400 | Hs.79137 | Carboxyl Methyltransferase, Aspartate, A | 5.6 |
| 100618 | HT2710 | Hs.114599 | Collagen, Type Viii, Alpha 1 | 7.5 |
| 100661 | HT3018 | Hs.132748 | Ribosomal Protein L39 Homolog | 4.4 |
| 100667 | HT3127 | Hs.169610 | Epican, Alt. Splice 11 | 4.6 |
| 100668 | HT3938 | Hs.169610 | Epican, Alt. Splice 12 | 4.4 |
| 100676 | HT3742 | Hs.287820 | Fibronectin, Alt. Splice 1 | 9 |
| 100775 | HT26388 | Hs.89603 | Mucin 1, Epithelial, Alt. Splice 9 | 4.7 |
| 100783 | HT4018 | Hs.191356 | Basic Transcription Factor, 44 Kda Subun | 13.7 |
| 100829 | HT4343 | Hs.278544 | Cytosolic Acetoacetyl-Coenzyme A Thio | 10.6 |
| 100830 | HT4344 | Hs.4756 | Rad2 | 5.5 |
| 100840 | HT4392 | Hs.183418 | Protein Kinase Pitslre, Alpha, Alt. Splice | 4.1 |
| 100850 | HT417 | Hs.297939 | Cathepsin B | 4 |
| 100866 | HT4582 | Hs.75113 | Transcription Factor Iiia | 4.9 |
| 100906 | HT5158 | Hs.5398 | Guanosine 5′-Monophosphate Synthase | 8.7 |
| 100914 | HT511 | Hs.324178 | Ras Inhibitor Inf | 7.2 |
| 100916 | HT544 | Hs.73946 | Endothelial Cell Growth Factor 1 | 5.9 |
| 100945 | HT884 | Hs.180686 | Oncogene E6-Ap, Papillomavirus | 4.6 |
| 100975 | J02923 | Hs.76506 | lymphocyte cytosolic protein 1 (L-plastin | 30.1 |
| 100988 | J03589 | Hs.76480 | ubiquitin-like 4 | 8.3 |
| 100996 | J03909 | Hs.14623 | interferon; gamma-inducible protein 30 | 6.9 |
| 100999 | J03934 | Hs.80706 | diaphorase (NADH/NADPH) (cytochrom | 4.3 |
| 101011 | J04430 | Hs.1211 | acid phosphatase 5; tartrate resistant | 5.9 |
| 101017 | J04599 | Hs.821 | biglycan | 5.1 |
| 101031 | J05070 | Hs.151738 | matrix metalloproteinase 9 (gelatinase B; | 37.2 |
| 101038 | J05249 | Hs.79411 | replication protein A2 (32 kD) | 6.1 |
| 101054 | K02405 | Hs.73931 | Human MHC class II HLA-DQ-beta mRN | 4.3 |
| 101061 | K03515 | Hs.180532 | glucose phosphate isomerase | 4.3 |
| 101091 | L06132 | Hs.149155 | voltage-dependent anion channel 1 | 7.4 |
| 101097 | L06797 | Hs.89414 | chemokine (C-X-C motif); receptor 4 (fus | 4.6 |
| 101104 | L07615 | Hs.169266 | Human neuropeptide Y receptor Y1 (NPY | 18.3 |
| 101143 | L12723 | Hs.90093 | heat shock 70 kD protein 4 | 17.4 |
| 101152 | L13800 | Hs.9884 | Homo sapiens liver expressed protein gen | 7.6 |
| 101183 | L19779 | Hs.795 | H2A histone family; member O | 10.9 |
| 101216 | L25876 | Hs.84113 | cyclin-dependent kinase inhibitor 3 (CDK | 7.4 |
| 101233 | L29008 | Hs.878 | sorbitol dehydrogenase | 14.6 |
| 101247 | L33801 | Hs.78802 | glycogen synthase kinase 3 beta | 7.5 |
| 101282 | L38810 | Hs.79387 | proteasome (prosome; macropain) 26S sub | 4.4 |
| 101326 | L42572 | Hs.78504 | inner membrane protein; mitochondrial (m | 5.8 |
| 101332 | L47276 | Hs.156346 | Homo sapiens (cell line HL-6) alpha topo | 18.9 |
| 101348 | L77213 | Hs.30954 | phosphomevalonate kinase | 7.5 |
| 101352 | L77701 | Hs.16297 | COX17 (yeast) homolog; cytochrome c ox | 9.3 |
| 101378 | M13755 | Hs.833 | interferon-stimulated protein; 15 kDa | 18.1 |
| 101396 | M15796 | Hs.78996 | proliferating cell nuclear antigen | 8.6 |
| 101404 | M16342 | Hs.182447 | heterogeneous nuclear ribonucleoprotein C | 4.5 |
| 101439 | M20902 | Hs.268571 | apolipoprotein C-I | 6.1 |
| 101464 | M22538 | Hs.51299 | NADH dehydrogenase (ubiquinone) flavo | 8.7 |
| 101469 | M22877 | Hs.169248 | Human somatic cytochrome c (HCS) gene | 4.2 |
| 101472 | M22960 | Hs.118126 | protective protein for beta-galactosidase ( | 6.5 |
| 101478 | M23379 | Hs.758 | RAS p21 protein activator (GTPase activa | 14 |
| 101484 | M24594 | Hs.20315 | interferon-induced protein 56 | 9.2 |
| 101539 | M30818 | Hs.926 | myxovirus (influenza) resistance 2; homol | 5.1 |
| 101540 | M30938 | Hs.84981 | X-ray repair complementing defective rep | 4.7 |
| 101544 | M31169 | | Human propionyl-CoA carboxylase beta-s | 5.5 |
| 101552 | M31642 | Hs.82314 | hypoxanthine phosphoribosyltransferase 1 | 8.5 |
| 101580 | M34677 | Hs.83363 | DNA segment on chromosome X (unique) | 4.5 |
| 101600 | M37583 | Hs.119192 | H2A histone family; member Z | 5.7 |
| 101663 | M60750 | Hs.2178 | H2B histone family; member A | 5.8 |
| 101664 | M60752 | Hs.121017 | H2A histone family; member A | 13.5 |
| 101667 | M60858 | Hs.79110 | nucleolin | 4 |
| 101684 | M63256 | Hs.75124 | cerebellar degeneration-related protein (62 | 7.6 |
| 101702 | M64929 | Hs.179574 | protein phosphatase 2 (formerly 2A); regu | 4.2 |
| 101754 | M77142 | Hs.239489 | TIA1 cytotoxic granule-associated RNA-b | 4.5 |
| 101758 | M77836 | Hs.79217 | pyrroline-5-carboxylate reductase 1 | 5.7 |
| 101767 | M81057 | Hs.180884 | carboxypeptidase B1 (tissue) | 21.7 |
| 101770 | M81601 | Hs.78869 | transcription elongation factor A (SII); 1 | 4.6 |
| 101791 | M83822 | Hs.62354 | cell division cycle 4-like | 9.7 |
| 101803 | M86546 | Hs.155691 | pre-B-cell leukemia transcription factor 1 | 5.5 |
| 101809 | M86849 | Hs.323733 | Homo sapiens connexin 26 (GJB2) mRNA | 22.5 |
| 101839 | M93036 | Hs.692 | membrane component; chromosomal 4; su | 4 |
| 101851 | M94250 | Hs.82045 | midkine (neurite growth-promoting factor | 7.6 |
| 101888 | M99701 | Hs.95243 | transcription elongation factor A (SII)-like | 11.4 |
| 101973 | S82597 | Hs.80120 | UDP-N-acetyl-alpha-D-galactosamine: po | 4.6 |
| 101991 | U00968 | Hs.166 | Human SREBP-1 mRNA; complete cds | 4.1 |
| 102009 | U02680 | Hs.82643 | protein tyrosine kinase 9 | 4.4 |
| 102025 | U03911 | Hs.78934 | mutS (E. coli) homolog 2 (colon cancer; n | 4 |
| 102047 | U07158 | Hs.83734 | syntaxin 4A (placental) | 6.1 |
| 102051 | U07550 | Hs.1197 | heat shock 10 kD protein 1 (chaperonin 10 | 4.4 |
| 102083 | U10323 | Hs.75117 | interleukin enhancer binding factor 2; 45 k | 10.4 |
| 102095 | U11313 | Hs.75760 | sterol carrier protein 2 | 9.5 |
| 102130 | U15009 | Hs.1575 | small nuclear ribonucleoprotein D3 polyp | 6.6 |
| 102133 | U15173 | Hs.155596 | BCL2/adenovirus E1B 19 kD-interacting p | 4.3 |
| 102148 | U16954 | Hs.75823 | ALL1-fused gene from chromosome 1q | 6.9 |
| 102179 | U19713 | Hs.76364 | allograft inflammatory factor 1 | 4.8 |
| 102180 | U19718 | Hs.83551 | microfibrillar-associated protein 2 | 7.2 |
| 102193 | U20758 | Hs.313 | secreted phosphoprotein 1 (osteopontin; b | 7.2 |
| 102198 | U21090 | Hs.74598 | polymerase (DNA directed); delta 2; regu | 4.3 |
| 102202 | U21931 | Hs.574 | fructose-bisphosphatase 1 | 4.5 |
| 102209 | U22970 | Hs.265827 | interferon; alpha-inducible protein (clone | 9.9 |
| 102211 | U23070 | Hs.78776 | putative transmembrane protein | 4.9 |
| 102220 | U24389 | Hs.65436 | lysyl oxidase-like 1 | 8.5 |
| 102224 | U24704 | Hs.148495 | proteasome (prosome; macropain) 26S sub | 5.4 |
| 102234 | U26312 | Hs.278554 | chromobox homolog 3 (Drosophila HP1 g | 7.7 |
| 102250 | U28014 | Hs.74122 | caspase 4; apoptosis-related cysteine prot | 5.4 |
| 102260 | U28386 | Hs.159557 | karyopherin alpha 2 (RAG cohort 1; impo | 6.3 |
| 102261 | U28488 | Hs.155935 | complement component 3a receptor 1 | 5.7 |
| 102273 | U30888 | Hs.75981 | ubiquitin specific protease 14 (tRNA-guan | 6.1 |
| 102298 | U32849 | Hs.54483 | N-myc (and STAT) interactor | 4.1 |
| 102302 | U33052 | Hs.69171 | protein kinase C-like 2 | 4.3 |
| 102305 | U33286 | Hs.90073 | chromosome segregation 1 (yeast homolo | 5.4 |
| 102320 | U34683 | Hs.82327 | glutathione synthetase | 4.1 |
| 102330 | U35451 | Hs.77254 | chromobox homolog 1 (Drosophila HP1 b | 4 |
| 102348 | U37519 | Hs.87539 | aldehyde dehydrogenase 8 | 9.4 |
| 102361 | U39400 | Hs.75859 | chromosome 11 open reading frame 4 | 5.2 |
| 102362 | U39412 | Hs.75932 | N-ethylmaleimide-sensitive factor attachm | 9.3 |
| 102369 | U39840 | Hs.299867 | hepatocyte nuclear factor 3; alpha | 7.7 |
| 102395 | U41767 | Hs.92208 | a disintegrin and metalloproteinase domai | 10.4 |
| 102409 | U43286 | Hs.118725 | selenophosphate synthetase 2 | 6.2 |
| 102418 | U43923 | Hs.79058 | suppressor of Ty (S. cerevisiae) 4 homolog | 4.1 |
| 102425 | U44772 | Hs.3873 | palmitoyl-protein thioesterase (ceroid-lipo | 4.8 |
| 102457 | U48807 | Hs.2359 | dual specificity phosphatase 4 | 6.3 |
| 102465 | U49352 | Hs.81548 | 2; 4-dienoyl CoA reductase 1; mitochondri | 9.4 |
| 102495 | U51240 | Hs.79356 | Lysosomal-associated multispanning mem | 6.5 |
| 102534 | U56833 | Hs.198307 | von Hippel-Lindau binding protein 1 | 8.6 |
| 102546 | U57877 | Hs.3577 | succinate dehydrogenase complex; subuni | 4.3 |
| 102549 | U58046 | Hs.198899 | eukaryotic translation initiation factor 3; s | 6.3 |
| 102557 | U58766 | Hs.264428 | tissue specific transplantation antigen P35 | 5 |
| 102562 | U59309 | Hs.75653 | fumarate hydratase | 6 |
| 102568 | U59877 | Hs.223025 | RAB31; member RAS oncogene family | 9.1 |
| 102580 | U60808 | Hs.152981 | CDP-diacylglycerol synthase (phosphatid | 7.9 |
| 102581 | U61145 | Hs.77256 | enhancer of zeste (Drosophila) homolog 2 | 7.6 |
| 102590 | U62136 | Hs.79300 | Homo sapiens enterocyte differentiation a | 7 |
| 102591 | U62325 | Hs.324125 | amyloid beta (A4) precursor protein-bindi | 4 |
| 102592 | U62389 | Hs.11223 | Human putative cytosolic NADP-depende | 5 |
| 102617 | U65928 | Hs.198767 | Jun activation domain binding protein | 6.1 |
| 102618 | U65932 | Hs.81071 | extracellular matrix protein 1 | 23.2 |
| 102638 | U67319 | Hs.9216 | caspase 7; apoptosis-related cysteine prot | 8.9 |
| 102663 | U70322 | Hs.168075 | karyopherin (importin) beta 2 | 7.1 |
| 102666 | U70660 | Hs.279910 | ATX1 (antioxidant protein 1; yeast) homo | 4.7 |
| 102679 | U72661 | Hs.11342 | ninjurin 1; nerve injury-induced protein-1 | 4.7 |
| 102687 | U73379 | Hs.93002 | ubiquitin carrier protein E2-C | 7.7 |
| 102704 | U76638 | Hs.54089 | BRCA1 associated RING domain 1 | 5.6 |
| 102705 | U77180 | Hs.50002 | small inducible cytokine subfamily A (Cy | 11.8 |
| 102721 | U79241 | Hs.118666 | Human clone 23759 mRNA; partial cds | 15 |
| 102729 | U79254 | Hs.181311 | asparaginyl-tRNA synthetase | 5 |
| 102739 | U79282 | Hs.155572 | Human clone 23801 mRNA sequence | 6 |
| 102742 | U79293 | Hs.159264 | Human clone 23948 mRNA sequence | 13.1 |
| 102761 | U82130 | Hs.118910 | tumor susceptibility gene 101 | 7 |
| 102788 | U86602 | Hs.74407 | nucleolar protein p40 | 4.1 |
| 102790 | U87269 | Hs.154196 | E4F transcription factor 1 | 7.1 |
| 102801 | U89606 | Hs.38041 | pyridoxal (pyridoxine; vitamin B6) kinase | 4.7 |
| 102808 | U90426 | Hs.179606 | nuclear RNA helicase; DECD variant of D | 7.5 |
| 102817 | U90904 | Hs.83724 | Human clone 23773 mRNA sequence | 15.2 |
| 102823 | U90914 | Hs.5057 | carboxypeptidase D | 6.6 |
| 102827 | U91327 | Hs.6456 | chaperonin containing TCP1; subunit 2 (b | 6 |
| 102838 | U94592 | Hs.80658 | Human uncoupling protein homolog (UCP | 6.1 |
| 102841 | U95006 | Hs.37616 | Human D9 splice variant B mRNA; comp | 4.2 |
| 102844 | U96113 | Hs.324275 | Homo sapiens Nedd-4-like ubiquitin-prot | 6.8 |
| 102868 | X02419 | Hs.77274 | plasminogen activator; urokinase | 4 |
| 102907 | X06985 | Hs.202833 | heme oxygenase (decycling) 1 | 22.7 |
| 102919 | X12447 | | aldolase A; fructose-bisphosphate | 9.9 |
| 102929 | X13238 | Hs.74649 | cytochrome c oxidase subunit Vlc | 5.4 |
| 102973 | X16663 | Hs.14601 | hematopoietic cell-specific Lyn substrate | 4.8 |
| 102983 | X17620 | Hs.118638 | non-metastatic cells 1; protein (NM23A) | 4.6 |
| 102985 | X17644 | Hs.2707 | G1 to S phase transition 1 | 20.6 |
| 103003 | X52003 | Hs.1406 | trefoil factor 1 (breast cancer; estrogen-ind | 10.7 |
| 103018 | X53296 | Hs.81134 | interleukin 1 receptor antagonist | 5.8 |
| 103023 | X53793 | Hs.117950 | multifunctional polypeptide similar to SA | 4 |
| 103036 | X54925 | Hs.83169 | matrix metalloproteinase 1 (interstitial col | 7.3 |
| 103060 | X57766 | Hs.155324 | matrix metalloproteinase 11 (stromelysin | 17.8 |
| 103073 | X59417 | Hs.74077 | proteasome (prosome; macropain) subunit | 5.6 |
| 103075 | X59543 | Hs.2934 | ribonucleotide reductase M1 polypeptide | 4.2 |
| 103080 | X59798 | Hs.82932 | cyclin D1 (PRAD1: parathyroid adenomat | 6.7 |
| 103094 | X60787 | Hs.296281 | interleukin enhancer binding factor 1 | 5.7 |
| 103105 | X61970 | Hs.76913 | proteasome (prosome; macropain) subunit | 5.8 |
| 103121 | X63679 | Hs.4147 | translocating chain-associating membrane | 4.2 |
| 103149 | X66363 | Hs.171834 | PCTAIRE protein kinase 1 | 12 |
| 103180 | X69433 | Hs.5337 | isocitrate dehydrogenase 2 (NADP+); mit | 18.9 |
| 103182 | X69819 | Hs.99995 | intercellular adhesion molecule 3 | 10.7 |
| 103188 | X70040 | Hs.2942 | macrophage stimulating 1 receptor (c-met | 4.1 |
| 103191 | X70218 | Hs.2903 | protein phosphatase 4 (formerly X); cataly | 10.7 |
| 103193 | X70476 | Hs.75724 | coatomer protein complex; subunit beta 2 | 8.2 |
| 103194 | X70649 | Hs.78580 | DEAD/H (Asp-Glu-Ala-Asp/His) box pol | 13.7 |
| 103195 | X70940 | Hs.2642 | eukaryotic translation elongation factor 1 | 13.4 |
| 103206 | X72755 | Hs.77367 | monokine induced by gamma interferon | 15.1 |
| 103207 | X72790 | | Human endogenous retrovirus mRNA for | 5.3 |
| 103208 | X72841 | Hs.31314 | retinoblastoma-binding protein 7 | 12.3 |
| 103216 | X74262 | Hs.16003 | retinoblastoma-binding protein 4 | 4.1 |
| 103226 | X75042 | Hs.44313 | v-rel avian reticuloendotheliosis viral onco | 6.9 |
| 103230 | X75861 | Hs.74637 | testis enhanced gene transcript | 7.9 |
| 103262 | X78565 | Hs.289114 | hexabrachion (tenascin C; cytotactin) | 5 |
| 103278 | X79882 | Hs.80680 | lung resistance-related protein | 5.7 |
| 103297 | X81788 | Hs.9078 | immature colon carcinoma transcript 1 | 4.6 |
| 103302 | X82103 | Hs.3059 | coatomer protein complex; subunit beta | 4.5 |
| 103316 | X83301 | Hs.324728 | SMA5 | 7.1 |
| 103330 | X85373 | Hs.77496 | small nuclear ribonucleoprotein polypepti | 4 |
| 103349 | X89059 | | serine/threonine kinase 9 | 4.7 |
| 103352 | X89398 | Hs.78853 | uracil-DNA glycosylase | 5.3 |
| 103364 | X90872 | Hs.279929 | SULT1C sulfotransferase | 4 |
| 103374 | X91788 | Hs.84974 | chloride channel; nucleotide-sensitive; 1A | 4.2 |
| 103380 | X92396 | Hs.24167 | synaptobrevin-like 1 | 13.6 |
| 103395 | X94754 | Hs.279946 | methionine-tRNA synthetase | 14.2 |
| 103402 | X95404 | Hs.180370 | cofilin 1 (non-muscle) | 4.6 |
| 103410 | X96506 | Hs.295362 | DR1-associated protein 1 (negative cofact | 8.3 |
| 103420 | X97065 | Hs.173497 | Sec23 (S. cerevisiae) homolog B | 4.9 |
| 103421 | X97074 | Hs.119591 | adaptor-related protein complex 2; sigma | 5 |
| 103427 | X97303 | Hs.250655 | H. sapiens mRNA for Ptg-12 protein | 7 |
| 103430 | X97544 | Hs.20716 | translocase of inner mitochondrial membr | 4.5 |
| 103438 | X98263 | Hs.152720 | M-phase phosphoprotein 6 | 4.5 |
| 103464 | Y00285 | Hs.76473 | insulin-like growth factor 2 receptor | 4.2 |
| 103470 | Y00796 | Hs.174103 | integrin; alpha L (antigen CD11A (p180); | 4.5 |
| 103494 | Y08991 | Hs.83050 | phosphatidylinositol 3-kinase-associated p | 4.1 |
| 103505 | Y09912 | Hs.33102 | transcription factor AP-2 beta (activating | 4.5 |
| 103547 | Z14982 | Hs.180062 | proteasome (prosome; macropain) subunit | 4.3 |
| 103551 | Z15115 | Hs.75248 | topoisomerase (DNA) II beta (180 kD) | 4 |
| 103565 | Z22548 | Hs.146354 | thioredoxin-dependent peroxide reductase | 7.6 |
| 103587 | Z29083 | Hs.82128 | 5T4 oncofetal trophoblast glycoprotein | 14.6 |
| 103621 | Z47727 | Hs.150675 | polymerase (RNA) II (DNA directed) pol | 6.3 |
| 103622 | Z48042 | Hs.278672 | membrane component; chromosome 11; s | 4.4 |
| 103658 | Z74615 | Hs.172928 | collagen; type I; alpha 1 | 5.9 |
| 103680 | Z93784 | | Homo sapiens DNA sequence from PAC | 4.4 |
| 103772 | AA092473 | Hs.278554 | chromobox homolog 3 (Drosophila HP1 g | 4.9 |
| 103774 | AA092898 | Hs.92918 | ESTs; Weakly similar to R07G3.8 [C. eleg | 6.1 |
| 103821 | AA157623 | Hs.198793 | KIAA0750 gene product | 23.3 |
| 103835 | AA172215 | Hs.93748 | ESTs; Moderately similar to TRANSCRIP | 4 |
| 103886 | AA236384 | Hs.105737 | ESTs; Weakly similar to gene 9306 protei | 4.9 |
| 103890 | AA236843 | Hs.72085 | ESTs; Weakly similar to unknown [S. cere | 7.8 |
| 103892 | AA243523 | Hs.239189 | ESTs | 4.8 |
| 104054 | AA393432 | Hs.7100 | hypothetical protein | 5.3 |
| 104115 | AA428090 | Hs.26102 | ESTs | 28.7 |
| 104136 | AA442669 | Hs.268371 | zv68f6.r1 Soares_total_fetus_Nb2HF8_9w | 5.7 |
| 104147 | AA451992 | Hs.283037 | ESTs; Highly similar to HSPC039 protein | 6.9 |
| 104173 | AA476564 | Hs.76561 | ESTs; Weakly similar to finger protein HZ | 5.2 |
| 104181 | AA479521 | Hs.283740 | ESTs | 7.8 |
| 104183 | AA480838 | Hs.114309 | ESTs | 5.1 |
| 104192 | AA486946 | Hs.21321 | Homo sapiens mRNA; cDNA DKFZp564 | 4.3 |
| 104209 | AB000221 | Hs.16530 | small inducible cytokine subfamily A (Cy | 12.3 |
| 104234 | AB002357 | Hs.168212 | kinesin family member 3B | 6.2 |
| 104271 | C01687 | Hs.7381 | ATP synthase; H+ transporting; mitochon | 4.2 |
| 104278 | C02582 | Hs.109253 | ESTs; Highly similar to N-terminal acetyl | 4.5 |
| 104307 | D52818 | Hs.111680 | endosulfine alpha | 4.7 |
| 104309 | D55869 | Hs.284123 | Homo sapiens mRNA full length insert cD | 4.2 |
| 104370 | H19378 | Hs.21851 | Homo sapiens mRNA; cDNA DKFZp586 | 6.4 |
| 104446 | L44497 | Hs.7351 | ESTs | 4.9 |
| 104453 | M19169 | Hs.123114 | cystatin SN | 11.6 |
| 104476 | N33807 | Hs.324275 | protease; serine; 15 | 5.6 |
| 104558 | R56678 | Hs.88959 | Human DNA sequence from clone 967N2 | 6.3 |
| 104592 | R81003 | Hs.325820 | serine protease; umbilical endothelium | 13.6 |
| 104634 | AA004274 | Hs.19151 | ESTs | 6.3 |
| 104636 | AA004415 | Hs.106106 | ESTs | 10.1 |
| 104658 | AA007145 | Hs.27268 | Homo sapiens mRNA; cDNA DKFZp564 | 4.3 |
| 104667 | AA007234 | Hs.30098 | ESTs | 16.6 |
| 104675 | AA009596 | Hs.301553 | ESTs; Moderately similar to !!!! ALU SU | 4.6 |
| 104767 | AA025534 | Hs.8852 | ESTs | 4.8 |
| 104785 | AA027163 | Hs.7942 | ESTs | 8.1 |
| 104791 | AA029046 | Hs.301871 | ESTs; Moderately similar to cAMP induc | 10.9 |
| 104804 | AA031357 | Hs.31803 | ESTs; Weakly similar to N-WASP [H. sap | 5.5 |
| 104807 | AA032147 | Hs.23296 | ESTs | 10.4 |
| 104837 | AA039469 | Hs.21126 | ESTs; Weakly similar to KIAA0299 [H. s | 4.6 |
| 104849 | AA040270 | Hs.241507 | Homo sapiens mRNA; cDNA DKFZp564 | 4.3 |
| 104867 | AA045481 | Hs.225979 | Human gene from PACs 37M17 and 305B | 4.5 |
| 104884 | AA053021 | Hs.14511 | SCO (cytochrome oxidase deficient; yeast | 4.7 |
| 104906 | AA055809 | Hs.26802 | ESTs; Weakly similar to phosphoprotein [ | 8.8 |
| 104919 | AA057193 | Hs.25252 | ESTs | 5.5 |
| 104921 | AA057839 | Hs.1508 | ESTs | 4.2 |
| 104926 | AA058846 | Hs.33363 | DKFZP434N093 protein | 7 |
| 104938 | AA064627 | Hs.318725 | ESTs; Highly similar to CGI-72 protein [H | 7.1 |
| 104943 | AA065217 | Hs.114218 | ESTs | 5.7 |
| 104957 | AA074919 | Hs.10026 | ESTs; Weakly similar to ORF YJL063c [S | 4.7 |
| 104961 | AA076672 | Hs.33905 | ESTs | 5.5 |
| 104968 | AA084602 | Hs.29669 | ESTs | 4.3 |
| 104975 | AA086071 | Hs.50758 | chromosome-associated polypeptide C | 8.3 |
| 104977 | AA088228 | Hs.18272 | ESTs | 6.2 |
| 104978 | AA088458 | Hs.19322 | ESTs | 6.7 |
| 104987 | AA101723 | Hs.11861 | ESTs | 9.2 |
| 105002 | AA113266 | Hs.182704 | ESTs; Moderately similar to alternatively | 6.9 |
| 105012 | AA116036 | Hs.9329 | chromosome 20 open reading frame 1 | 10.7 |
| 105019 | AA121879 | Hs.9280 | proteasome (prosome; macropain) subunit | 5.7 |
| 105029 | AA126855 | Hs.13268 | ESTs | 4.4 |
| 105033 | AA127964 | Hs.274329 | TP53 target gene 1 | 6.3 |
| 105035 | AA128486 | Hs.8859 | ESTs | 6.5 |
| 105039 | AA130349 | Hs.36475 | ESTs | 4 |
| 105062 | AA134968 | Hs.36529 | ESTs | 4.3 |
| 105076 | AA142858 | Hs.37810 | ESTs | 6.4 |
| 105087 | AA147884 | Hs.9812 | ESTs | 9.2 |
| 105091 | AA148859 | Hs.179909 | ESTs; Weakly similar to !!!! ALU SUBFA | 5.7 |
| 105093 | AA149051 | Hs.32405 | ESTs | 6.3 |
| 105107 | AA152302 | Hs.25035 | DKFZP566G223 protein | 6.2 |
| 105127 | AA158132 | Hs.301957 | ESTs; Weakly similar to contains similari | 5.7 |
| 105132 | AA159501 | Hs.247280 | HBV associated factor | 4.2 |
| 105143 | AA165333 | Hs.24808 | ESTs | 4.7 |
| 105154 | AA171736 | Hs.35947 | methyl-CpG binding domain protein 4 | 9 |
| 105162 | AA176690 | Hs.4084 | KIAA1025 protein | 9.1 |
| 105186 | AA191512 | Hs.28005 | Homo sapiens mRNA; cDNA DKFZp564 | 19.3 |
| 105209 | AA205072 | Hs.227743 | KIAA0980 protein | 7.4 |
| 105223 | AA211388 | Hs.7750 | ESTs | 5.1 |
| 105252 | AA227428 | Hs.9728 | ESTs; Weakly similar to KIAA0512 prote | 11.1 |
| 105253 | AA227448 | Hs.5003 | KIAA0456 protein | 6.4 |
| 105261 | AA227871 | Hs.6361 | MEK partner 1 | 9.1 |
| 105263 | AA227926 | Hs.6682 | ESTs | 6.7 |
| 105274 | AA228122 | Hs.281866 | ATPase; H+ transporting; lysosomal (vacu | 5.3 |
| 105297 | AA233451 | Hs.183858 | transcriptional intermediary factor 1 | 8.7 |
| 105309 | AA233790 | Hs.4104 | ESTs; Weakly similar to cDNA EST yk38 | 7.4 |
| 105312 | AA233854 | Hs.23348 | S-phase kinase-associated protein 2 (p45) | 5.8 |
| 105342 | AA235286 | Hs.157078 | ESTs | 4.5 |
| 105376 | AA236559 | Hs.8768 | ESTs; Weakly similar to !!!! ALU SUBFA | 5.8 |
| 105386 | AA236950 | Hs.8115 | ESTs | 5.5 |
| 105397 | AA242868 | Hs.7395 | ESTs; Weakly similar to house-keeping p | 7.7 |
| 105399 | AA243007 | Hs.16420 | ESTs; Highly similar to SH3 domain-bind | 5.6 |
| 105400 | AA243052 | Hs.65648 | RNA binding motif protein 8 | 5.8 |
| 105404 | AA243303 | Hs.21187 | ESTs | 9.1 |
| 105409 | AA243562 | Hs.301855 | ESTs | 4.4 |
| 105436 | AA252172 | Hs.237856 | ESTs; Moderately similar to cAMP induc | 5.1 |
| 105483 | AA255874 | Hs.23458 | ESTs | 4.9 |
| 105493 | AA256268 | Hs.10283 | ESTs | 6 |
| 105495 | AA256317 | Hs.28785 | Homo sapiens mRNA; cDNA DKFZp586 | 5.2 |
| 105496 | AA256323 | Hs.301997 | DKFZP434N126 protein | 8.7 |
| 105500 | AA256485 | Hs.222399 | CGI-96 protein | 9.5 |
| 105507 | AA256678 | Hs.226318 | ESTs; Moderately similar to CCR4-associ | 4.1 |
| 105538 | AA258860 | Hs.32597 | ring finger protein (C3H2C3 type) 6 | 4.1 |
| 105544 | AA261954 | Hs.24678 | ESTs | 8 |
| 105546 | AA262032 | Hs.268281 | ESTs; Weakly similar to 62D9.a [D. melan | 8.1 |
| 105549 | AA262417 | Hs.5415 | ESTs | 4.6 |
| 105551 | AA262477 | Hs.25292 | ribonuclease HI; large subunit | 9.1 |
| 105560 | AA262783 | Hs.306915 | ESTs | 4.5 |
| 105565 | AA278302 | Hs.18349 | ESTs; Weakly similar to partial CDS [C. e | 4.2 |
| 105566 | AA278323 | Hs.17481 | Homo sapiens clone 24606 mRNA sequen | 11.9 |
| 105575 | AA278717 | Hs.12772 | ESTs | 5.9 |
| 105584 | AA279012 | Hs.3454 | ESTs; Weakly similar to KIAA0665 prote | 4.4 |
| 105596 | AA279418 | Hs.18490 | ESTs | 4 |
| 105604 | AA279787 | Hs.15467 | ESTs; Moderately similar to putative pho | 5.6 |
| 105610 | AA279991 | Hs.99872 | ESTs; Weakly similar to trithorax homolo | 5.3 |
| 105621 | AA280865 | Hs.6375 | Homo sapiens mRNA; cDNA DKFZp564 | 4.8 |
| 105627 | AA281245 | Hs.23317 | ESTs | 7.5 |
| 105638 | AA281599 | Hs.247817 | Homo sapiens mRNA for for histone H2B | 5.9 |
| 105645 | AA282138 | Hs.11325 | ESTs | 6.4 |
| 105650 | AA282347 | Hs.25635 | ESTs; Highly similar to HSPC003 [H. sap | 11.3 |
| 105666 | AA283930 | Hs.34906 | ESTs | 4.7 |
| 105674 | AA284755 | Hs.279789 | CDW52 antigen (CAMPATH-1 antigen) | 8 |
| 105687 | AA286809 | Hs.28423 | ESTs | 7.1 |
| 105700 | AA287643 | Hs.35254 | ESTs; Weakly similar to hypothetical pro | 4.9 |
| 105705 | AA290767 | Hs.101282 | Homo sapiens mRNA; cDNA DKFZp434 | 8 |
| 105709 | AA291268 | Hs.26761 | DKFZP586L0724 protein | 6.8 |
| 105731 | AA292711 | Hs.29131 | ESTs | 6.4 |
| 105753 | AA299789 | Hs.110857 | ESTs | 7 |
| 105774 | AA348014 | Hs.23412 | ESTs | 7.1 |
| 105784 | AA350771 | Hs.17850 | ESTs | 13.4 |
| 105791 | AA358038 | Hs.14368 | SH3-binding domain glutamic acid-rich p | 4.3 |
| 105807 | AA393803 | Hs.16869 | ESTs; Moderately similar to COLLAGEN | 5.3 |
| 105808 | AA393808 | Hs.286131 | KIAA0438 gene product | 4.1 |
| 105812 | AA394126 | Hs.20814 | ESTs; Highly similar to CGI-27 protein [H | 14.6 |
| 105813 | AA394140 | Hs.18585 | ESTs | 4.9 |
| 105819 | AA397920 | Hs.28783 | Homo sapiens mRNA; cDNA DKFZp564 | 4.9 |
| 105870 | AA399623 | Hs.101067 | ESTs | 4.8 |
| 105874 | AA400074 | Hs.171118 | ESTs | 4 |
| 105896 | AA400999 | Hs.7838 | Human ring zinc-finger protein (ZNF127- | 4.8 |
| 105934 | AA404248 | Hs.16577 | ESTs | 5.2 |
| 105935 | AA404277 | Hs.263727 | ESTs; Weakly similar to bisphosphate 3′- | 4 |
| 105966 | AA406105 | Hs.5344 | adaptor-related protein complex 1; gamma | 8.3 |
| 105974 | AA406321 | Hs.6224 | KIAA0895 protein | 4.6 |
| 105990 | AA410336 | Hs.29403 | ESTs; Weakly similar to PROBABLE AT | 4.5 |
| 105995 | AA410510 | Hs.5345 | ESTs | 4.9 |
| 106000 | AA410972 | Hs.20726 | ESTs | 5.8 |
| 106007 | AA411462 | Hs.11042 | ESTs; Weakly similar to veli 1 [H. sapiens | 6.9 |
| 106016 | AA411819 | Hs.8164 | KIAA0898 protein | 5 |
| 106034 | AA412473 | Hs.14928 | ESTs | 6.6 |
| 106042 | AA412700 | Hs.169895 | ubiquitin-conjugating enzyme E2L 6 | 4.6 |
| 106057 | AA417067 | Hs.289074 | ESTs | 4.5 |
| 106065 | AA417558 | Hs.25206 | ESTs | 12.3 |
| 106070 | AA417761 | Hs.5957 | Homo sapiens clone 24416 mRNA sequen | 5 |
| 106103 | AA421104 | Hs.12094 | ESTs | 15.4 |
| 106126 | AA424006 | Hs.22972 | ESTs; Moderately similar to H5AR [M. m | 6.4 |
| 106154 | AA425304 | Hs.6994 | ESTs | 5.1 |
| 106157 | AA425367 | Hs.34892 | ESTs | 11.1 |
| 106166 | AA425872 | Hs.19561 | NADH dehydrogenase (ubiquinone) 1 alp | 19.3 |
| 106204 | AA428024 | Hs.21479 | ESTs | 4.7 |
| 106210 | AA428239 | Hs.10338 | ESTs | 5.7 |
| 106220 | AA428582 | Hs.32196 | ESTs; Moderately similar to metargidin p | 7.7 |
| 106236 | AA429951 | Hs.21104 | ESTs | 8 |
| 106240 | AA430074 | Hs.18552 | ESTs; Weakly similar to Ylr218cp [S. cere | 4.4 |
| 106263 | AA431462 | Hs.28329 | ESTs | 4.9 |
| 106288 | AA435536 | Hs.24336 | ESTs | 8.8 |
| 106293 | AA435591 | Hs.301444 | signal sequence receptor; gamma (transloc | 8.7 |
| 106310 | AA436244 | Hs.17240 | ESTs | 4.5 |
| 106317 | AA436568 | Hs.108124 | ESTs | 4 |
| 106328 | AA436705 | Hs.28020 | KIAA0766 gene product | 4.4 |
| 106341 | AA441798 | Hs.5243 | ESTs; Moderately similar to plL2 hypoth | 23.7 |
| 106348 | AA442253 | Hs.10702 | ESTs | 4.7 |
| 106350 | AA442763 | Hs.194698 | cyclin B2 | 6.1 |
| 106371 | AA443923 | Hs.170310 | ESTs | 6.8 |
| 106389 | AA446949 | Hs.6236 | ESTs | 4.7 |
| 106394 | AA447223 | Hs.25320 | Homo sapiens clone 25142 mRNA sequen | 4.4 |
| 106426 | AA448282 | Hs.16206 | ESTs; Weakly similar to F55C12.5 [C. ele | 4.5 |
| 106459 | AA449741 | Hs.4029 | glioma-amplified sequence-41 | 4.8 |
| 106462 | AA449912 | Hs.30532 | ESTs; Highly similar to CGI-77 protein [H | 5.2 |
| 106468 | AA450047 | Hs.14770 | ESTs | 6.8 |
| 106479 | AA450351 | Hs.75251 | ESTs | 12.4 |
| 106494 | AA452108 | Hs.18387 | transcription factor AP-2 alpha (activating | 4.5 |
| 106503 | AA452411 | Hs.29679 | ESTs; Highly similar to mediator [H. sapie | 5.1 |
| 106507 | AA452584 | Hs.267819 | protein phosphatase 1; regulatory (inhibito | 4.9 |
| 106533 | AA453786 | Hs.145998 | ESTs | 8.3 |
| 106568 | AA455970 | Hs.28285 | patched related protein translocated in ren | 7.6 |
| 106586 | AA456598 | Hs.57787 | ESTs | 8.2 |
| 106589 | AA456646 | Hs.28661 | ESTs | 4.8 |
| 106606 | AA457730 | Hs.283437 | Homo sapiens clone 23851 mRNA sequen | 4.4 |
| 106611 | AA458904 | Hs.26267 | ESTs; Weakly similar to torsinA [H. sapie | 7 |
| 106614 | AA458934 | Hs.256150 | ESTs | 4.5 |
| 106628 | AA459657 | Hs.12311 | Homo sapiens clone 23570 mRNA sequen | 6.5 |
| 106637 | AA459961 | Hs.250824 | ESTs | 5.5 |
| 106644 | AA460239 | Hs.12680 | ESTs | 4.4 |
| 106664 | AA460969 | Hs.7510 | mitogen-activated protein kinase kinase ki | 8.4 |
| 106698 | AA463745 | Hs.29403 | ESTs; Weakly similar to PROBABLE AT | 5.3 |
| 106719 | AA465171 | Hs.236844 | ESTs | 5.6 |
| 106726 | AA465339 | Hs.3886 | ESTs | 10.1 |
| 106747 | AA476473 | Hs.171957 | triple functional domain (PTPRF interacti | 10.4 |
| 106759 | AA477263 | Hs.25584 | ESTs | 4.2 |
| 106765 | AA477717 | Hs.306117 | interleukin 13 receptor; alpha 1 | 6.9 |
| 106784 | AA478558 | Hs.227913 | API5-like 1 | 5.1 |
| 106831 | AA482014 | Hs.29463 | centrin; EF-hand protein; 3 (CDC31 yeast | 5.1 |
| 106836 | AA482112 | Hs.238707 | ESTs | 4.8 |
| 106840 | AA482548 | Hs.5534 | ESTs | 10.3 |
| 106856 | AA486183 | Hs.285123 | ESTs; Weakly similar to similar to oxyste | 6.2 |
| 106865 | AA487228 | Hs.19479 | ESTs | 4.5 |
| 106878 | AA488872 | Hs.12314 | Homo sapiens mRNA; cDNA DKFZp586 | 7.9 |
| 106888 | AA489101 | Hs.24734 | oxysterol binding protein | 6.4 |
| 106895 | AA489665 | Hs.25245 | ESTs | 4.6 |
| 106909 | AA490323 | Hs.250747 | SUMO-1 activating enzyme subunit 1 | 4.2 |
| 106919 | AA490885 | Hs.21766 | ESTs | 12.3 |
| 106920 | AA490899 | Hs.296323 | ESTs | 6.2 |
| 106941 | AA496204 | Hs.237971 | ESTs | 4 |
| 106942 | AA496347 | Hs.31314 | retinoblastoma-binding protein 7 | 4.8 |
| 106948 | AA496788 | Hs.21077 | KIAA0532 protein | 4 |
| 106968 | AA504631 | Hs.26813 | ESTs; Weakly similar to hypothetical 43.2 | 4.4 |
| 106973 | AA505141 | Hs.11923 | Human DNA sequence from clone 167A1 | 5.4 |
| 106980 | AA521121 | Hs.8858 | bromodomain adjacent to zinc finger dom | 4.1 |
| 106981 | AA521157 | Hs.74101 | ESTs | 5.7 |
| 106998 | AA598461 | Hs.195464 | insulin-like growth factor binding protein | 18.7 |
| 107008 | AA598710 | Hs.23740 | ESTs | 6.2 |
| 107028 | AA599214 | Hs.24143 | ESTs | 4.1 |
| 107032 | AA599472 | Hs.247309 | succinate-CoA ligase; GDP-forming; beta | 5.3 |
| 107052 | AA600134 | Hs.12482 | glyceronephosphate O-acyltransferase | 4.8 |
| 107053 | AA600147 | Hs.5741 | ESTs; Weakly similar to NADH-cytochro | 5.8 |
| 107056 | AA600310 | Hs.18720 | programmed cell death 8 (apoptosis-induc | 4.9 |
| 107080 | AA609210 | Hs.19221 | ESTs | 8.4 |
| 107102 | AA609723 | Hs.30652 | ESTs | 8 |
| 107109 | AA609943 | Hs.32793 | ESTs | 9.5 |
| 107129 | AA620553 | Hs.4756 | flap structure-specific endonuclease 1 | 4.9 |
| 107132 | AA620598 | Hs.9052 | ESTs | 5.3 |
| 107136 | AA620795 | Hs.8207 | ESTs | 4 |
| 107140 | AA620889 | Hs.170088 | ESTs | 6.7 |
| 107151 | AA621169 | Hs.8687 | ESTs | 19 |
| 107159 | AA621340 | Hs.10600 | ESTs; Weakly similar to ORF YKR081c [ | 8.1 |
| 107174 | AA621714 | Hs.25338 | ESTs | 8.5 |
| 107217 | D51095 | Hs.35861 | DKFZP586E1621 protein | 7.2 |
| 107252 | D59971 | Hs.25925 | ESTs | 7.9 |
| 107295 | T34527 | Hs.80120 | UDP-N-acetyl-alpha-D-galactosamine: po | 5.6 |
| 107299 | T40327 | Hs.30661 | lung resistance-related protein | 8.4 |
| 107324 | T81665 | Hs.278422 | DKFZP586G1122 protein | 7.5 |
| 107372 | U85625 | Hs.8297 | ribonuclease 6 precursor | 4.7 |
| 107373 | U85773 | Hs.154695 | phosphomannomutase 2 | 4.8 |
| 107481 | W58247 | Hs.279766 | Homo sapiens kinesin superfamily motor | 6.3 |
| 107531 | Y13936 | Hs.17883 | protein phosphatase 1G (formerly 2C); ma | 8.3 |
| 107859 | AA024835 | Hs.47584 | potassium voltage-gated channel; delayed | 7.3 |
| 107890 | AA026030 | Hs.61311 | ESTs; Weakly similar to CALPAIN 2; LA | 7.3 |
| 107908 | AA026894 | Hs.42826 | ESTs | 4.9 |
| 108039 | AA041341 | Hs.46670 | ESTs | 5.4 |
| 108040 | AA041551 | Hs.159971 | ESTs | 8.4 |
| 108102 | AA046424 | Hs.49433 | ESTs; Weakly similar to HYPOTHETICA | 6.6 |
| 108217 | AA058686 | Hs.62588 | ESTs | 7.7 |
| 108255 | AA063157 | Hs.172608 | ESTs | 4 |
| 108358 | AA071514 | Hs.1634 | ESTs | 4 |
| 108609 | AA100694 | Hs.69499 | Human DNA sequence from BAC 15E1 o | 5.5 |
| 108647 | AA112396 | Hs.44276 | ESTs; Moderately similar to HOMEOBO | 14.3 |
| 108676 | AA115562 | Hs.274417 | Homo sapiens mRNA; cDNA DKFZp564 | 5.2 |
| 108687 | AA120785 | Hs.54347 | ESTs | 5.6 |
| 108695 | AA121315 | Hs.70823 | KIAA1077 protein | 10.5 |
| 108733 | AA126422 | | zn84f1.s1 Stratagene lung carcinoma 9372 | 4.4 |
| 108774 | AA128125 | Hs.71040 | ESTs; Moderately similar to CELL GROW | 4.6 |
| 108828 | AA131584 | Hs.273344 | DKFZP564O0463 protein | 5.5 |
| 108872 | AA134063 | Hs.111680 | ESTs | 7.2 |
| 108884 | AA134958 | Hs.293591 | ESTs | 11.3 |
| 108893 | AA135894 | Hs.194691 | retinoic acid induced 3 | 8.9 |
| 109008 | AA156360 | Hs.87128 | ESTs | 14.7 |
| 109010 | AA156460 | Hs.44229 | dual specificity phosphatase 12 | 4.9 |
| 109011 | AA156542 | Hs.72127 | ESTs | 4.6 |
| 109042 | AA159525 | Hs.71779 | Homo sapiens DNA from chromosome 19 | 7.2 |
| 109086 | AA166695 | Hs.270737 | tumor necrosis factor (ligand) superfamily | 4 |
| 109090 | AA167006 | Hs.70499 | ESTs | 5.9 |
| 109101 | AA167708 | Hs.52184 | ESTs | 4.2 |
| 109112 | AA169379 | Hs.257924 | ESTs | 4 |
| 109160 | AA179387 | Hs.301997 | DKFZP434N126 protein | 4 |
| 109166 | AA179845 | Hs.73625 | RAB6 interacting; kinesin-like (rabkinesin | 13.6 |
| 109178 | AA181600 | Hs.283707 | ESTs | 11.8 |
| 109179 | AA181902 | Hs.192789 | ESTs; Weakly similar to !!!! ALU SUBFA | 5.4 |
| 109261 | AA195255 | Hs.61779 | ESTs | 6.7 |
| 109270 | AA195515 | Hs.3585 | ESTs; Weakly similar to alternatively spli | 4.9 |
| 109277 | AA196332 | Hs.86043 | ESTs | 5.4 |
| 109313 | AA206800 | Hs.86276 | ESTs; Moderately similar to zinc finger p | 5.5 |
| 109415 | AA227219 | Hs.110826 | trinucleotide repeat containing 9 | 20.1 |
| 109454 | AA232255 | Hs.295232 | ESTs | 4.7 |
| 109467 | AA232904 | Hs.63187 | ESTs | 6.8 |
| 109481 | AA233342 | Hs.289069 | ESTs; Weakly similar to WD40 protein C | 10.6 |
| 109508 | AA233892 | Hs.55902 | ESTs; Weakly similar to !!!! ALU SUBFA | 8 |
| 109514 | AA234087 | Hs.262346 | ESTs; Weakly similar to ORF2: function | 8.2 |
| 109572 | F02027 | Hs.171937 | ESTs | 4.8 |
| 109632 | F04165 | Hs.235873 | ESTs; Weakly similar to K11C4.2 [C. eleg | 5.2 |
| 109644 | F04477 | Hs.291531 | ESTs; Moderately similar to GLYCERAL | 6.6 |
| 109703 | F09684 | Hs.24792 | ESTs; Weakly similar to ORF YOR283w | 7.1 |
| 109726 | F10009 | Hs.9196 | ESTs | 5 |
| 109747 | F10161 | Hs.22969 | ESTs | 4.7 |
| 109799 | F10770 | Hs.180378 | Homo sapiens clone 669 unknown mRNA | 4.5 |
| 109814 | F10979 | Hs.153106 | Homo sapiens clone 23728 mRNA sequen | 8.7 |
| 110189 | H20543 | Hs.6278 | DKFZP586B1621 protein | 16.6 |
| 110240 | H25577 | Hs.176588 | ESTs; Weakly similar to CYTOCHROME | 6.2 |
| 110280 | H29285 | Hs.32468 | ESTs | 4.5 |
| 110520 | H56965 | Hs.4082 | yr09f06.s1 Soares fetal liver spleen 1NFL | 5.7 |
| 110561 | H59617 | Hs.5199 | ESTs; Weakly similar to UBIQUITIN-CO | 19.5 |
| 110707 | H95079 | Hs.15617 | ESTs; Weakly similar to !!!! ALU SUBFA | 6.2 |
| 110734 | H98714 | Hs.24131 | ESTs | 30.2 |
| 110770 | N22262 | Hs.131705 | ESTs | 5.8 |
| 110780 | N23174 | Hs.22891 | solute carrier family 7 (cationic amino aci | 8.2 |
| 110787 | N24716 | Hs.12244 | ESTs; Weakly similar to C44B9.1 [C. eleg | 6.7 |
| 110794 | N25262 | Hs.27931 | ESTs | 5.9 |
| 110799 | N26101 | Hs.323401 | Human ring zinc-finger protein (ZNF127- | 4 |
| 110818 | N29454 | Hs.27552 | ESTs; Weakly similar to putative p150 [H | 4.3 |
| 110839 | N30856 | Hs.30246 | solute carrier family 19 (thiamine transpo | 12.8 |
| 110844 | N31952 | Hs.167531 | Homo sapiens mRNA full length insert cD | 10.1 |
| 110854 | N32919 | Hs.27931 | ESTs | 4.7 |
| 110856 | N33063 | | ESTs; Weakly similar to S164 [H. sapiens | 4.2 |
| 110860 | N33438 | Hs.170065 | ESTs | 12.5 |
| 110897 | N39148 | Hs.6880 | DKFZP434D156 protein | 4 |
| 110915 | N46252 | Hs.29724 | ESTs | 23.2 |
| 110935 | N48787 | Hs.305979 | protease inhibitor 1 (anti-elastase); alpha- | 4.8 |
| 110970 | N51374 | Hs.96870 | Homo sapiens mRNA full length insert cD | 5.4 |
| 111006 | N53375 | Hs.166146 | Homer; neuronal immediate early gene; 3 | 4.7 |
| 111008 | N53388 | Hs.7222 | ESTs | 13.3 |
| 111018 | N54067 | Hs.3628 | mitogen-activated protein kinase kinase ki | 5.7 |
| 111084 | N59543 | Hs.15456 | PDZ domain containing 1 | 8.3 |
| 111100 | N62522 | Hs.20450 | ESTs | 14.3 |
| 111125 | N63823 | Hs.269115 | ESTs | 7.9 |
| 111132 | N64378 | Hs.83293 | ESTs; Highly similar to unknown function | 4.4 |
| 111139 | N64683 | Hs.290943 | ESTs | 6 |
| 111164 | N66857 | Hs.14808 | ESTs; Weakly similar to !!!! ALU CLASS | 4.1 |
| 111172 | N67102 | Hs.21851 | Homo sapiens mRNA; cDNA DKFZp586 | 5.5 |
| 111178 | N67227 | Hs.24633 | ESTs | 5.7 |
| 111179 | N67239 | Hs.10760 | ESTs | 37 |
| 111181 | N67278 | Hs.171802 | ESTs; Weakly similar to hypothetical pro | 5.6 |
| 111184 | N67437 | Hs.243901 | Homo sapiens mRNA; cDNA DKFZp564 | 8.7 |
| 111221 | N68869 | Hs.15119 | ESTs | 7.3 |
| 111223 | N68921 | Hs.297939 | ESTs; Weakly similar to neogenin [H. sap | 9 |
| 111229 | N69113 | Hs.110855 | ESTs | 8.9 |
| 111241 | N69514 | Hs.288880 | ESTs; Weakly similar to CGI-82 protein [ | 6.9 |
| 111268 | N70481 | Hs.26118 | Homo sapiens clone 24766 mRNA sequen | 4.5 |
| 111295 | N73275 | Hs.21275 | ESTs; Weakly similar to ubiquitin-conjug | 5.6 |
| 111299 | N73808 | Hs.24936 | ESTs | 8.5 |
| 111336 | N79565 | Hs.29894 | ESTs | 6.7 |
| 111357 | N91023 | Hs.87128 | ESTs | 15 |
| 111370 | N92915 | Hs.94631 | brefeldin A-inhibited guanine nucleotide-e | 5.2 |
| 111806 | R33468 | Hs.279008 | ESTs | 10 |
| 111825 | R35885 | Hs.286148 | stromal antigen 1 | 4.5 |
| 111836 | R36228 | Hs.25119 | ESTs | 7.2 |
| 111890 | R38678 | Hs.12365 | ESTs | 17.3 |
| 111923 | R39995 | Hs.25925 | Homo sapiens clone 23860 mRNA sequen | 7.3 |
| 111942 | R40576 | Hs.21590 | ESTs | 9.2 |
| 111987 | R42036 | Hs.6763 | KIAA0942 protein | 10.6 |
| 112101 | R44793 | Hs.296341 | adenylyl cyclase-associated protein 2 | 5.3 |
| 112134 | R46025 | Hs.7413 | ESTs | 17.4 |
| 112197 | R49482 | Hs.5637 | ESTs | 4.4 |
| 112244 | R51309 | Hs.70823 | KIAA1077 protein | 11 |
| 112253 | R51818 | | Homo sapiens mRNA; cDNA DKFZp566 | 9.3 |
| 112305 | R54822 | Hs.26244 | ESTs | 4.4 |
| 112449 | R63802 | Hs.124186 | ring finger protein 2 | 6.3 |
| 112483 | R66534 | Hs.285885 | ESTs | 4.9 |
| 112519 | R68631 | Hs.11861 | ESTs | 14.3 |
| 112610 | R79392 | Hs.23643 | ESTs | 5.2 |
| 112693 | R88741 | Hs.91065 | ESTs; Moderately similar to proliferation | 4.6 |
| 112751 | R93507 | Hs.8207 | ESTs | 5.6 |
| 112801 | R97486 | Hs.157160 | protein kinase; DNA-activated; catalytic p | 8.7 |
| 112869 | T03313 | Hs.4747 | dyskeratosis congenita 1; dyskerin | 5.9 |
| 112871 | T03352 | Hs.12285 | ESTs | 5.8 |
| 112908 | T10065 | Hs.3530 | TLS-associated serine-arginine protein | 4.1 |
| 112966 | T17119 | Hs.102548 | glucocorticoid receptor DNA binding fact | 5.7 |
| 112971 | T17185 | Hs.83883 | ESTs | 6.4 |
| 112995 | T23528 | Hs.7155 | ESTs; Weakly similar to TYKi protein [M | 9.1 |
| 113047 | T25867 | Hs.7549 | ESTs | 5.4 |
| 113075 | T34660 | Hs.6986 | ESTs; Weakly similar to !!!! ALU SUBFA | 5.7 |
| 113117 | T47819 | Hs.159153 | ESTs | 5.8 |
| 113206 | T58044 | Hs.241471 | ESTs; Moderately similar to !!!! ALU SU | 6.4 |
| 113248 | T63857 | | yc16e1.s1 Stratagene lung (#93721) Homo | 4.6 |
| 113260 | T64896 | Hs.287420 | ESTs | 6.9 |
| 113277 | T65797 | Hs.11774 | protein (peptidyl-prolyl cis/trans isomeras | 5.6 |
| 113278 | T65802 | Hs.11135 | yc11h10.s1 Stratagene lung (#937210) Ho | 4.5 |
| 113440 | T86121 | Hs.191445 | ESTs | 6.4 |
| 113523 | T90037 | Hs.95549 | ESTs | 6.4 |
| 113604 | T92735 | Hs.296083 | ESTs | 8.7 |
| 113702 | T97307 | | ESTs; Moderately similar to !!!! ALU SU | 9.5 |
| 113783 | W19222 | Hs.7041 | ESTs; Weakly similar to !!!! ALU SUBFA | 5.2 |
| 113794 | W37382 | Hs.11090 | ESTs | 11.9 |
| 113808 | W44735 | Hs.9286 | ESTs | 16.7 |
| 113811 | W44928 | Hs.6994 | ESTs | 4 |
| 113822 | W47350 | Hs.17466 | retinoic acid receptor responder (tazaroten | 4.8 |
| 113823 | W47388 | Hs.55099 | rab6 GTPase activating protein (GAP and | 4 |
| 113836 | W56792 | Hs.12040 | ESTs; Weakly similar to KIAA0881 prote | 4.1 |
| 113857 | W65477 | Hs.5297 | Homo sapiens mRNA; cDNA DKFZp564 | 4.3 |
| 113886 | W72471 | Hs.23920 | ESTs | 4.6 |
| 113895 | W73738 | Hs.12921 | ESTs | 7.1 |
| 113923 | W80763 | Hs.3849 | ESTs; Weakly similar to FK506-binding p | 6.8 |
| 113931 | W81205 | Hs.3496 | ESTs | 6.1 |
| 113950 | W85765 | Hs.30504 | Homo sapiens mRNA; cDNA DKFZp434 | 14 |
| 113970 | W86748 | Hs.8109 | ESTs | 15 |
| 114051 | W94942 | Hs.177534 | dual specificity phosphatase 10 | 5.4 |
| 114057 | W96222 | Hs.34192 | ESTs | 4.8 |
| 114086 | Z38266 | Hs.288649 | Homo sapiens PAC clone DJ0777O23 fro | 5.1 |
| 114098 | Z38347 | Hs.118338 | ESTs; Weakly similar to similar to S. cere | 6.2 |
| 114109 | Z38435 | Hs.184108 | ribosomal protein L21 | 4.6 |
| 114124 | Z38595 | Hs.125019 | ESTs; Highly similar to KIAA0886 prote | 22 |
| 114138 | Z38763 | Hs.15740 | amyloid beta (A4) precursor protein-bindi | 8.8 |
| 114149 | Z38814 | Hs.27196 | ESTs | 4 |
| 114162 | Z38909 | Hs.22265 | ESTs | 7.2 |
| 114177 | Z39062 | Hs.23740 | ESTs | 5.3 |
| 114196 | Z39211 | Hs.150926 | fucose-1-phosphate guanylyltransferase | 4.4 |
| 114208 | Z39301 | Hs.7859 | ESTs | 5.1 |
| 114250 | Z39897 | Hs.13297 | ESTs | 7.2 |
| 114251 | Z39898 | Hs.21948 | ESTs | 14.7 |
| 114292 | Z40715 | Hs.184641 | delta-6 fatty acid desaturase | 19.4 |
| 114297 | Z40758 | Hs.173091 | DKFZP434K151 protein | 8.9 |
| 114334 | Z41342 | Hs.22941 | ESTs | 13.7 |
| 114460 | AA024604 | Hs.26102 | ESTs | 10.1 |
| 114471 | AA028074 | Hs.104613 | ESTs | 5.7 |
| 114480 | AA032243 | Hs.151678 | UDP-N-acetyl-alpha-D-galactosamine: po | 7.3 |
| 114518 | AA046407 | Hs.106469 | suppressor of var1 (S. cerevisiae) 3-like 1 | 4.3 |
| 114542 | AA055768 | Hs.293380 | ESTs | 11.7 |
| 114549 | AA056484 | Hs.292833 | ESTs | 7.3 |
| 114652 | AA101416 | Hs.107149 | ESTs; Weakly similar to PTB-ASSOCIAT | 6.1 |
| 114673 | AA113303 | Hs.95583 | transmembrane 4 superfamily member (te | 4.3 |
| 114698 | AA126951 | Hs.110857 | ESTs; Highly similar to putative DNA-dir | 7.1 |
| 114767 | AA148885 | Hs.154443 | minichromosome maintenance deficient (S | 5.3 |
| 114799 | AA159323 | Hs.109929 | ESTs | 4.2 |
| 114804 | AA160363 | Hs.269956 | ESTs | 4.8 |
| 114811 | AA161161 | Hs.95907 | multiple inositol polyphosphate phosphata | 7.1 |
| 114821 | AA165313 | Hs.55468 | ESTs | 4.4 |
| 114852 | AA235035 | Hs.38260 | ESTs; Moderately similar to ubiquitin spe | 5 |
| 114901 | AA236276 | Hs.196437 | ESTs: Weakly similar to R26660_1; parti | 16.9 |
| 114902 | AA236359 | Hs.39504 | ESTs | 5.1 |
| 114940 | AA243012 | Hs.75928 | ESTs | 8.5 |
| 114965 | AA250737 | Hs.72472 | ESTs | 35.1 |
| 115047 | AA252627 | Hs.82916 | homeo box B5 | 5.7 |
| 115054 | AA252863 | Hs.87729 | ESTs | 6.2 |
| 115061 | AA253217 | Hs.41271 | ESTs | 13 |
| 115082 | AA255557 | Hs.198269 | NADH dehydrogenase (ubiquinone) 1 alp | 28.2 |
| 115116 | AA256486 | Hs.62275 | ESTs | 8.8 |
| 115140 | AA258030 | Hs.279938 | ESTs; Weakly similar to supported by GE | 4.1 |
| 115205 | AA262470 | Hs.284216 | ESTs | 8.3 |
| 115206 | AA262491 | Hs.186572 | ESTs | 5.1 |
| 115239 | AA278650 | Hs.73291 | ESTs; Weakly similar to similar to the bet | 4.6 |
| 115242 | AA278755 | Hs.283732 | ESTs | 8.3 |
| 115249 | AA278961 | Hs.71124 | ESTs | 10.1 |
| 115259 | AA279071 | Hs.13453 | splicing factor 3b; subunit 1; 155 kD | 9.5 |
| 115285 | AA279799 | Hs.293736 | ESTs | 5.8 |
| 115291 | AA279943 | Hs.122579 | ESTs | 5.1 |
| 115357 | AA281793 | Hs.72988 | ESTs | 5 |
| 115377 | AA282247 | Hs.193063 | ESTs | 6.1 |
| 115400 | AA283198 | Hs.89113 | ESTs | 4.9 |
| 115439 | AA284561 | Hs.193090 | ESTs | 5.8 |
| 115471 | AA287138 | Hs.59346 | ESTs; Weakly similar to ASPARTYL-TR | 11.7 |
| 115506 | AA292537 | Hs.45207 | Human DNA sequence from clone 620E1 | 6.8 |
| 115522 | AA331393 | Hs.47378 | ESTs | 5.8 |
| 115572 | AA398392 | Hs.59594 | ESTs; Weakly similar to F33G12.3 gene p | 9.7 |
| 115587 | AA399264 | Hs.283037 | ESTs; Highly similar to HSPC039 protein | 8.7 |
| 115600 | AA400247 | Hs.42173 | ESTs | 4 |
| 115612 | AA400948 | Hs.71243 | ESTs; Weakly similar to zinc finger prote | 8.4 |
| 115646 | AA404352 | Hs.305971 | ESTs | 5.3 |
| 115652 | AA405098 | Hs.38178 | ESTs | 16.1 |
| 115657 | AA405620 | Hs.55158 | ESTs; Weakly similar to weak similarity t | 4.7 |
| 115658 | AA405625 | Hs.183056 | Human DNA sequence from clone 34B21 | 5.1 |
| 115675 | AA406546 | Hs.82065 | Homo sapiens mRNA; cDNA DKFZp564 | 20.5 |
| 115721 | AA417102 | Hs.90960 | ESTs | 4.8 |
| 115763 | AA421560 | | ESTs | 7 |
| 115764 | AA421562 | Hs.91011 | anterior gradient 2 (Xenepus laevis) homo | 41.6 |
| 115835 | AA428576 | Hs.41371 | ESTs | 4.2 |
| 115844 | AA430124 | Hs.7773 | ESTs | 11.9 |
| 115875 | AA433943 | Hs.43946 | ESTs; Weakly similar to Weak similarity | 33.5 |
| 115888 | AA435839 | Hs.76591 | KIAA0887 protein | 7.2 |
| 115922 | AA441911 | Hs.71869 | ESTs; Weakly similar to KIAA0926 prote | 5.1 |
| 115941 | AA443602 | Hs.46679 | ESTs | 4.8 |
| 115947 | AA443793 | Hs.94761 | ESTs | 8.3 |
| 115948 | AA443798 | Hs.43445 | poly(A)-specific ribonuclease (deadenylat | 13.5 |
| 115951 | AA443918 | Hs.301048 | cofilin 1 (non-muscle) | 7.5 |
| 115967 | AA446887 | Hs.42911 | ESTs | 8.8 |
| 115984 | AA447687 | Hs.91109 | ESTs | 13.1 |
| 116009 | AA449448 | Hs.44238 | ESTs | 5.5 |
| 116024 | AA451748 | Hs.83883 | Human DNA sequence from clone 718J7 | 7.5 |
| 116028 | AA452112 | Hs.42644 | thioredoxin-like | 12.7 |
| 116050 | AA453656 | Hs.88417 | ESTs | 7.2 |
| 116097 | AA456099 | Hs.176376 | ESTs | 11.8 |
| 116108 | AA457566 | Hs.28777 | ESTs | 4.5 |
| 116121 | AA459254 | Hs.48855 | ESTs | 4.5 |
| 116127 | AA459703 | Hs.279884 | v-myc avian myelocytomatosis viral onco | 4.3 |
| 116129 | AA459956 | Hs.49163 | ESTs; Highly similar to putative ribonucle | 7.6 |
| 116142 | AA460649 | Hs.39457 | ESTs | 4.8 |
| 116204 | AA465701 | Hs.108646 | ESTs | 6.8 |
| 116221 | AA478397 | Hs.50180 | ESTs | 4.9 |
| 116222 | AA478415 | Hs.89986 | ESTs | 4 |
| 116238 | AA479362 | Hs.47144 | DKFZP586N0819 protein | 4.6 |
| 116246 | AA479961 | Hs.250646 | ESTs; Highly similar to ubiquitin-conjuga | 4 |
| 116249 | AA480886 | Hs.86693 | ESTs | 18.5 |
| 116250 | AA480975 | Hs.44829 | ESTs | 10.8 |
| 116254 | AA481146 | Hs.41086 | ESTs; Weakly similar to OXYSTEROL-B | 9.1 |
| 116256 | AA481256 | Hs.88201 | ESTs; Weakly similar to lysophospholipa | 8.4 |
| 116264 | AA482594 | Hs.272239 | Homo sapiens mRNA; cDNA DKFZp586 | 7.2 |
| 116265 | AA482595 | Hs.55189 | ESTs; Weakly similar to F25B5.3 [C. eleg | 11.1 |
| 116282 | AA486550 | Hs.204501 | ESTs; Weakly similar to Wiskott-Aldrich | 6.2 |
| 116298 | AA489046 | Hs.94109 | ESTs | 4.9 |
| 116300 | AA489194 | Hs.159471 | ESTs; Weakly similar to snRNP protein B | 4.6 |
| 116327 | AA490959 | Hs.28005 | Homo sapiens mRNA; cDNA DKFZp564 | 5.8 |
| 116334 | AA491457 | Hs.48948 | ESTs | 4.3 |
| 116337 | AA496127 | Hs.44070 | ESTs | 8.4 |
| 116351 | AA504116 | Hs.82501 | Homo sapiens mRNA; cDNA DKFZp434 | 5.3 |
| 116357 | AA504806 | Hs.90797 | Homo sapiens clone 23620 mRNA sequen | 5.2 |
| 116415 | AA609204 | Hs.27973 | KIAA0874 protein | 6.6 |
| 116443 | AA620313 | Hs.190488 | ESTs; Weakly similar to KERATIN; TYP | 4.5 |
| 116470 | C13992 | Hs.83484 | ESTs | 4.5 |
| 116480 | C14088 | | glyceraldehyde-3-phosphate dehydrogena | 5.6 |
| 116578 | D51272 | Hs.75337 | nucleolar phosphoprotein p130 | 4.1 |
| 116579 | D51276 | Hs.81915 | leukemia-associated phosphoprotein p18 | 5.8 |
| 116626 | F02028 | Hs.81907 | ESTs | 4.9 |
| 116647 | F03069 | Hs.15395 | ESTs; Weakly similar to ARGINYL-TRN | 6.1 |
| 116674 | F04816 | Hs.92127 | ESTs | 10.6 |
| 116680 | F08813 | Hs.273829 | LINE retrotransposable element 1 | 4.2 |
| 116700 | F09983 | Hs.317589 | ESTs | 13 |
| 116724 | F13665 | Hs.65641 | ESTs | 8.5 |
| 116726 | F13681 | Hs.53913 | ESTs | 5.6 |
| 116732 | F13779 | Hs.165909 | ESTs | 11.6 |
| 116734 | F13789 | Hs.93796 | DKFZP586D2223 protein | 5.4 |
| 116760 | H11054 | Hs.155342 | protein kinase C; delta | 4.3 |
| 116780 | H22566 | Hs.30098 | ESTs | 5.7 |
| 116786 | H25836 | Hs.301527 | tumor necrosis factor (ligand) superfamily | 8.8 |
| 116787 | H28581 | Hs.15641 | ESTs | 8.6 |
| 116790 | H29532 | Hs.101174 | microtubule-associated protein tau | 22.2 |
| 116803 | H47357 | Hs.109701 | ESTs; Moderately similar to weak similar | 6.7 |
| 116877 | H68116 | Hs.168732 | ESTs | 6.5 |
| 116921 | H72948 | Hs.821 | biglycan | 20.7 |
| 117216 | N20083 | Hs.42792 | ESTs | 4.4 |
| 117232 | N20579 | Hs.61153 | ESTs | 7.4 |
| 117284 | N22162 | Hs.183779 | ESTs; Weakly similar to cDNA EST yk33 | 4.1 |
| 117344 | N24046 | Hs.210706 | ESTs | 7.4 |
| 117367 | N24954 | Hs.42502 | ESTs | 10.5 |
| 117392 | N26175 | Hs.93405 | ESTs | 5.8 |
| 117394 | N26257 | Hs.39871 | KIAA0727 protein | 8.4 |
| 117412 | N26722 | Hs.42645 | ESTs | 18.1 |
| 117498 | N31726 | Hs.44268 | ESTs; Highly similar to myelin gene expr | 5.8 |
| 117557 | N33920 | Hs.44532 | diubiquitin | 12.3 |
| 117634 | N36421 | Hs.13323 | ESTs; Weakly similar to SODIUM-AND | 4.4 |
| 117639 | N36923 | Hs.44833 | ESTs | 6 |
| 117754 | N47469 | Hs.59757 | ESTs | 7.6 |
| 117852 | N49408 | Hs.136102 | KIAA0853 protein | 5.9 |
| 117879 | N50050 | Hs.303025 | ESTs; Weakly similar to keratin; 67 K typ | 7.9 |
| 117924 | N51056 | Hs.38891 | ESTs | 7.9 |
| 117950 | N51394 | Hs.75478 | KIAA0956 protein | 5 |
| 117992 | N52000 | Hs.172089 | Homo sapiens mRNA; cDNA DKFZp586 | 7 |
| 118138 | N57773 | Hs.93560 | ESTs; Weakly similar to trg [R. norvegicu | 4.8 |
| 118215 | N62195 | Hs.77910 | 3-hydroxy-3-methylglutaryl-Coenzyme A | 13.4 |
| 118229 | N62339 | Hs.166254 | heat shock 90 kD protein 1; alpha | 5.4 |
| 118265 | N62827 | Hs.48645 | EST | 4.2 |
| 118336 | N63604 | Hs.47166 | ESTs | 7.2 |
| 118363 | N64168 | Hs.48938 | ESTs | 6 |
| 118429 | N66158 | Hs.74649 | ESTs | 4.1 |
| 118470 | N66769 | Hs.291033 | ESTs | 5.4 |
| 118472 | N66818 | Hs.42179 | ESTs | 10.8 |
| 118475 | N66845 | | ESTs; Weakly similar to !!!! ALU CLASS | 4.5 |
| 118493 | N67149 | Hs.50115 | ESTs | 5.3 |
| 118528 | N67889 | Hs.49397 | ESTs | 10.4 |
| 118542 | N68010 | Hs.49427 | ESTs | 7.9 |
| 118600 | N69222 | | ESTs | 9.2 |
| 118695 | N71781 | Hs.50081 | Homo sapiens mRNA full length insert cD | 9.8 |
| 118698 | N72113 | Hs.50187 | ESTs | 4.3 |
| 118901 | N90719 | Hs.94445 | ESTs | 8.1 |
| 118952 | N92966 | — | ESTs; Highly similar to CGI-90 protein [H | 12.5 |
| 118976 | N93629 | Hs.93391 | ESTs | 5 |
| 118986 | N94362 | Hs.125830 | ESTs | 7.3 |
| 118989 | N94439 | Hs.45105 | ESTs | 8.2 |
| 119027 | N99256 | Hs.114611 | ESTs | 5 |
| 119042 | R05316 | Hs.5472 | ESTs | 4 |
| 119075 | R36451 | Hs.287820 | fibronectin 1 | 6 |
| 119260 | T15916 | Hs.102950 | ESTs; Highly similar to coat protein gamm | 4.1 |
| 119271 | T16387 | Hs.65328 | ESTs | 12.1 |
| 119298 | T23820 | Hs.155478 | cyclin T2 | 5.6 |
| 119302 | T25725 | | ESTs | 14.3 |
| 119341 | T62571 | Hs.146388 | microtubule-associated protein 7 | 4 |
| 119495 | W35390 | Hs.55533 | ESTs | 5.3 |
| 119580 | W42451 | Hs.92260 | high-mobility group protein 2-like 1 | 5.6 |
| 119602 | W46286 | Hs.233694 | ESTs; Weakly similar to ZK1058.5 [C. ele | 6.5 |
| 119620 | W47620 | Hs.56009 | 2′-5′oligoadenylate synthetase 3 | 8.1 |
| 119676 | W60473 | Hs.57787 | ESTs | 5.5 |
| 119717 | W69134 | Hs.57987 | ESTs | 4.6 |
| 119729 | W69747 | Hs.94806 | KIAA1062 protein | 4 |
| 119805 | W73788 | Hs.43213 | ESTs | 4 |
| 119859 | W80702 | Hs.58461 | ESTs | 4.8 |
| 119867 | W80852 | Hs.250696 | KDEL (Lys-Asp-Glu-Leu) endoplasmic re | 4.2 |
| 119873 | W81129 | Hs.44865 | Homo sapiens mRNA; cDNA DKFZp586 | 4.8 |
| 119899 | W84767 | Hs.58698 | ESTs | 5.9 |
| 119940 | W86779 | Hs.272531 | DKFZP586B0319 protein | 9 |
| 119943 | W86835 | Hs.14158 | copine III | 4.8 |
| 119970 | W87812 | Hs.93581 | Homo sapiens mRNA; cDNA DKFZp586 | 4 |
| 120131 | Z38656 | Hs.75887 | coatomer protein complex; subunit alpha | 4.2 |
| 120150 | Z39549 | Hs.153746 | ESTs | 11 |
| 120206 | Z40805 | Hs.91668 | ESTs | 8.2 |
| 120241 | Z41815 | Hs.65946 | ESTs | 15.6 |
| 120255 | AA169752 | Hs.5672 | ESTs; Weakly similar to Similarity to Yea | 4.2 |
| 120314 | AA194166 | Hs.221040 | KIAA1038 protein | 6.8 |
| 120325 | AA195651 | Hs.104106 | ESTs | 15.2 |
| 120352 | AA211400 | Hs.193172 | ESTs | 6.8 |
| 120428 | AA236822 | Hs.173694 | KIAA1097 protein | 5.6 |
| 120524 | AA261852 | Hs.192905 | ESTs | 5.6 |
| 120528 | AA262107 | Hs.104413 | ESTs | 4.5 |
| 120571 | AA280738 | Hs.34892 | ESTs | 4.9 |
| 120649 | AA287115 | Hs.192843 | ESTs | 4.5 |
| 120655 | AA287347 | Hs.238205 | ESTs | 6.7 |
| 120668 | AA287833 | Hs.292913 | ESTs | 8.3 |
| 120712 | AA292654 | Hs.102506 | eukaryotic translation initiation factor 2 al | 4.6 |
| 120713 | AA292655 | Hs.96557 | ESTs | 10.6 |
| 120724 | AA293470 | Hs.100747 | ESTs | 5.4 |
| 120873 | AA358015 | | EST | 7.1 |
| 120885 | AA365515 | Hs.301872 | ESTs; Moderately similar to !!!! ALU SU | 4.6 |
| 120919 | AA381125 | Hs.301444 | ESTs | 8.2 |
| 120948 | AA397822 | Hs.104650 | ESTs; Highly similar to similar to mago n | 8.6 |
| 120969 | AA398116 | Hs.129206 | casein kinase 1; gamma 3 | 10.5 |
| 120977 | AA398155 | Hs.97600 | ESTs | 10.9 |
| 121103 | AA398936 | Hs.97697 | EST | 7.4 |
| 121291 | AA401753 | Hs.8186 | lung cancer candidate | 5.3 |
| 121320 | AA403008 | Hs.301927 | T-cell receptor; alpha (V; D; J; C) | 13.5 |
| 121463 | AA411745 | Hs.239681 | ESTs; Weakly similar to KIAA0554 prote | 8.9 |
| 121596 | AA416740 | Hs.174104 | ESTs | 22.6 |
| 121723 | AA419622 | Hs.104800 | ESTs; Weakly similar to Mouse 19.5 mRN | 8 |
| 121748 | AA421171 | Hs.234545 | ESTs | 5.6 |
| 122125 | AA434411 | Hs.98806 | ESTs | 5.3 |
| 122522 | AA449444 | Hs.98969 | ESTs | 4 |
| 122655 | AA454756 | Hs.97837 | ESTs | 4 |
| 122704 | AA456326 | Hs.99445 | ESTs | 6.2 |
| 122782 | AA459894 | Hs.99472 | ESTs | 5.3 |
| 122856 | AA463740 | Hs.75367 | Src-like-adapter | 13.1 |
| 122882 | AA465381 | Hs.108812 | ESTs; Weakly similar to B0041.5 [C. eleg | 5.5 |
| 122928 | AA476578 | Hs.101840 | ESTs | 6.3 |
| 122974 | AA478625 | Hs.194215 | ESTs | 6 |
| 122997 | AA479295 | Hs.106290 | Kelch motif containing protein | 12.5 |
| 123016 | AA480103 | Hs.323231 | ESTs; Weakly similar to alternatively spli | 4.4 |
| 123107 | AA486071 | Hs.104207 | ESTs | 8.3 |
| 123111 | AA486273 | Hs.191721 | ESTs | 4.2 |
| 123114 | AA486407 | Hs.129928 | ESTs; Moderately similar to KIAA0454 p | 5.2 |
| 123136 | AA487449 | Hs.194024 | ESTs | 4.2 |
| 123137 | AA487468 | Hs.100686 | ESTs; Weakly similar to secreted cement | 14.6 |
| 123169 | AA488892 | | ESTs; Weakly similar to Gag-Pol polypro | 4.5 |
| 123176 | AA489020 | Hs.69233 | ESTs | 5.2 |
| 123338 | AA504249 | Hs.187585 | ESTs | 4 |
| 123436 | AA598714 | Hs.223014 | protease; serine; 15 | 7.3 |
| 123442 | AA598803 | Hs.111496 | ESTs | 5.9 |
| 123449 | AA598899 | Hs.112493 | Homo sapiens mRNA; cDNA DKFZp564 | 4.1 |
| 123494 | AA599786 | Hs.112110 | ESTs | 4 |
| 123503 | AA600121 | Hs.293156 | ESTs | 12.8 |
| 123533 | AA608751 | | ESTs; Weakly similar to !!!! ALU SUBFA | 7.9 |
| 123619 | AA609200 | | ESTs | 23.1 |
| 123673 | AA609471 | Hs.158549 | ESTs | 6.6 |
| 123729 | AA609778 | Hs.278672 | membrane component; chromosome 11; s | 4.7 |
| 123819 | AA620636 | Hs.112264 | ESTs | 4 |
| 123960 | AA621785 | Hs.287733 | methylmalonate-semialdehyde dehydroge | 7.6 |
| 124000 | D57317 | Hs.74861 | activated RNA polymerase II transcription | 4.4 |
| 124006 | D60302 | Hs.270016 | ESTs | 20.6 |
| 124012 | D80240 | Hs.241471 | HUM5G11A Human fetal brain (TFujiwa | 6.7 |
| 124021 | F02859 | Hs.13974 | ESTs | 4.7 |
| 124049 | F10523 | Hs.74519 | primase; polypeptide 2A (58 kD) | 4.7 |
| 124059 | F13673 | Hs.283713 | ESTs | 7.7 |
| 124243 | H66710 | Hs.133525 | ESTs | 5.5 |
| 124308 | H93575 | Hs.241507 | Homo sapiens mRNA; cDNA DKFZp564 | 11.4 |
| 124314 | H94877 | Hs.215766 | GTP-binding protein | 13.7 |
| 124315 | H94892 | Hs.288757 | v-ral simian leukemia viral oncogene hom | 14 |
| 124350 | N21359 | Hs.101282 | Homo sapiens mRNA; cDNA DKFZp434 | 8.6 |
| 124352 | N21626 | Hs.102406 | ESTs | 7.2 |
| 124357 | N22401 | | yw37g07.s1 Morton Fetal Cochlea Homo | 5.2 |
| 124390 | N29325 | Hs.7535 | ESTs; Highly similar to COBW-like place | 7.9 |
| 124438 | N40188 | Hs.11090 | ESTs | 9.5 |
| 124447 | N48000 | | Homo sapiens mRNA; cDNA DKFZp586 | 4.8 |
| 124457 | N50114 | Hs.266175 | ESTs | 6.1 |
| 124539 | N63172 | Hs.146409 | cell division cycle 42 (GTP-binding prote | 5.6 |
| 124626 | N74604 | Hs.11090 | ESTs | 12.8 |
| 124632 | N79515 | Hs.306117 | interleukin 13 receptor; alpha 1 | 6.4 |
| 124644 | N91279 | Hs.109654 | ESTs; Moderately similar to outer membr | 8.3 |
| 124676 | R01037 | Hs.181013 | phosphoglycerate mutase 1 (brain) | 12.3 |
| 124677 | R01073 | | ESTs; Weakly similar to !!!! ALU CLASS | 5.4 |
| 124724 | R12405 | Hs.112423 | Homo sapiens mRNA; cDNA DKFZp586 | 6.6 |
| 124773 | R40923 | Hs.106604 | ESTs | 4.9 |
| 124777 | R41933 | | ESTs | 7.2 |
| 124792 | R44357 | Hs.48712 | ESTs; Weakly similar to cDNA EST EMB | 8.6 |
| 124857 | R63652 | Hs.137190 | ESTs | 4.9 |
| 124911 | R88992 | Hs.180612 | ESTs | 4.7 |
| 124955 | T10598 | Hs.324841 | ESTs; Weakly similar to !!!! ALU SUBFA | 4.4 |
| 124958 | T11134 | Hs.431 | murine leukemia viral (bmi-1) oncogene h | 12.6 |
| 125038 | T78089 | Hs.270134 | ESTs | 4.1 |
| 125092 | T92544 | Hs.137548 | CD84 antigen (leukocyte antigen) | 14.8 |
| 125132 | W15495 | Hs.129781 | chromosome 21 open reading frame 5 | 6.7 |
| 125144 | W37999 | Hs.24336 | ESTs | 4.8 |
| 125154 | W38419 | | ESTs | 5.3 |
| 125243 | W86423 | Hs.105413 | ESTs | 6.6 |
| 125279 | W93640 | Hs.4779 | ESTs; Moderately similar to similar to AD | 5.8 |
| 125299 | Z39436 | Hs.102720 | ESTs | 12.2 |
| 125303 | Z39821 | Hs.288193 | ESTs | 10.2 |
| 125304 | Z39833 | Hs.124940 | GTP-binding protein | 6.8 |
| 125474 | AA151216 | Hs.75103 | tyrosine 3-monooxygenase/tryptophan 5-m | 8 |
| 125509 | AA044232 | Hs.288967 | ESTs | 5.4 |
| 125580 | AA126504 | Hs.267812 | sorting nexin 4 | 4.1 |
| 125582 | AA507383 | Hs.74649 | cytochrome c oxidase subunit VIc | 11.5 |
| 125670 | AI432621 | Hs.82685 | CD47 antigen (Rh-related antigen; integri | 4 |
| 125698 | AA748483 | Hs.191356 | general transcription factor IIH; polypepti | 9.4 |
| 125745 | A1283493 | Hs.75722 | ribophorin II | 6.2 |
| 125852 | H09290 | Hs.76550 | Homo sapiens mRNA; cDNA DKFZp564 | 25.9 |
| 125972 | AA434562 | Hs.35406 | ESTs | 4.1 |
| 126160 | N90960 | Hs.265398 | ESTs; Weakly similar to transformation-r | 16.4 |
| 126257 | N99638 | Hs.124084 | tumor necrosis factor receptor superfamily | 9.5 |
| 126337 | AI066486 | Hs.40500 | similar to S. cerevisiae RER1 | 5.6 |
| 126405 | U46278 | Hs.122489 | ESTs | 7.5 |
| 126537 | W40262 | Hs.146310 | ESTs; Weakly similar to putative p150 [H | 4.1 |
| 126590 | W78968 | Hs.181307 | H3 histone; family 3A | 4.5 |
| 126712 | AA205862 | Hs.7942 | ESTs | 5.2 |
| 126721 | T72569 | Hs.125359 | Thy-1 cell surface antigen | 4.4 |
| 126764 | AI334393 | Hs.102178 | ESTs | 4.6 |
| 126804 | AI203334 | Hs.160628 | ESTs | 11.7 |
| 126819 | AA305536 | Hs.279607 | ESTs | 4 |
| 126877 | AI052047 | Hs.26102 | ESTs | 7 |
| 126991 | R31652 | Hs.821 | biglycan | 5.6 |
| 127479 | AA513722 | Hs.179729 | collagen; type X; alpha 1 (Schmid metaph | 14.3 |
| 127514 | AA826926 | Hs.204214 | ESTs | 4.5 |
| 127663 | W07286 | Hs.10340 | ESTs; Weakly similar to weak similarity t | 5.1 |
| 127677 | AA916752 | Hs.264190 | ESTs; Highly similar to MEM3 [M. muscu | 17.3 |
| 127814 | AA761755 | Hs.136713 | ESTs; Weakly similar to V4-1 [H. sapiens | 4.1 |
| 127997 | AI281549 | Hs.311054 | ESTs | 5.5 |
| 128092 | AA904617 | Hs.166229 | ESTs | 5.8 |
| 128218 | H02682 | Hs.292154 | ESTs; Moderately similar to recombinatio | 5.8 |
| 128466 | D59653 | Hs.241471 | EST | 7.4 |
| 128482 | U83908 | Hs.296251 | programmed cell death 4 | 5.8 |
| 128517 | AA280617 | Hs.100861 | ESTs; Weakly similar to p60 katanin [H. s | 8.3 |
| 128530 | AA504343 | Hs.183475 | Homo sapiens clone 25061 mRNA sequen | 6.6 |
| 128559 | AA226801 | Hs.101448 | metastasis associated 1 | 5.2 |
| 128574 | AA412048 | Hs.38260 | keratin 8 | 5.1 |
| 128595 | U31875 | Hs.152677 | short-chain alcohol dehydrogenase family | 27.1 |
| 128610 | L38608 | Hs.10247 | activated leucocyte cell adhesion molecule | 13.2 |
| 128629 | AA399187 | Hs.102708 | DKFZP434A043 protein | 6.7 |
| 128649 | AA142853 | Hs.103106 | Homo sapiens mRNA for G7b protein (G | 4.5 |
| 128651 | AA440990 | Hs.103135 | ESTs | 6.1 |
| 128653 | R48943 | Hs.10315 | solute carrier family 7 (cationic amino aci | 4.4 |
| 128656 | AA458542 | Hs.10326 | coatomer protein complex; subunit epsilon | 14.3 |
| 128717 | T30617 | Hs.104222 | Homo sapiens mRNA; cDNA DKFZp566 | 24.5 |
| 128727 | M64174 | Hs.50651 | Janus kinase 1 (a protein tyrosine kinase) | 7.3 |
| 128764 | N49308 | Hs.104938 | ESTs; Weakly similar to alpha 1(XVIII) c | 9.2 |
| 128781 | X85372 | Hs.105465 | small nuclear ribonucleoprotein polypepti | 5.4 |
| 128793 | W93562 | Hs.105749 | KIAA0553 protein | 4.6 |
| 128835 | W15528 | Hs.106390 | Homo sapiens mRNA; cDNA DKFZp586 | 4 |
| 128845 | AA455658 | Hs.10649 | basement membrane-induced gene | 6.9 |
| 128871 | AA400271 | Hs.106778 | Homo sapiens mRNA for putative Ca2+−t | 4.5 |
| 128922 | AA252023 | Hs.9589 | ESTs; Weakly similar to HRIHFB2157 [H | 6.4 |
| 128925 | D61676 | Hs.21851 | Homo sapiens mRNA; cDNA DKFZp586 | 6.4 |
| 128938 | AA410325 | Hs.107260 | ESTs | 7 |
| 128946 | N29353 | Hs.107318 | kynurenine 3-monooxygenase (kynurenin | 5.2 |
| 128948 | AA485655 | Hs.223025 | proteasome (prosome; macropain) subunit | 13.1 |
| 128955 | F10290 | Hs.185807 | Homo sapiens clone 24758 mRNA sequen | 5.8 |
| 129005 | AA460049 | Hs.13323 | ESTs; Weakly similar to SODIUM-AND | 12.6 |
| 129009 | AA131421 | Hs.75607 | ESTs | 9.8 |
| 129017 | H13108 | Hs.107968 | ESTs | 13.9 |
| 129057 | X62466 | Hs.276770 | CDW52 antigen (CAMPATH-1 antigen) | 10.7 |
| 129075 | AA129465 | Hs.83765 | ESTs | 4.7 |
| 129095 | L12350 | Hs.108623 | thrombospondin 2 | 4.4 |
| 129124 | AA234530 | Hs.108802 | N-ethylmaleimide-sensitive factor | 20.7 |
| 129160 | AA131252 | Hs.109007 | ESTs | 5.9 |
| 129164 | AA282183 | Hs.109045 | ESTs | 5.8 |
| 129180 | R40556 | Hs.318401 | ESTs; Highly similar to HSPC039 protein | 7.6 |
| 129224 | X89109 | Hs.109606 | coronin; actin-binding protein; 1A | 12 |
| 129229 | AA211941 | Hs.109643 | polyadenylate binding protein-interacting | 7.9 |
| 129240 | W24360 | Hs.237868 | interleukin 7 receptor | 5.3 |
| 129241 | AA435665 | Hs.109706 | ESTs; Moderately similar to HN1 [M. mus | 8.4 |
| 129243 | H88033 | Hs.109727 | KIAA0733 protein | 7.8 |
| 129247 | AA151574 | Hs.109733 | pilin-like transcription factor | 6.4 |
| 129259 | AA090695 | Hs.181385 | ESTs | 6.2 |
| 129270 | Z35227 | Hs.109918 | ras homolog gene family; member H | 5.4 |
| 129281 | AA026318 | Hs.289101 | glucose regulated protein; 58 kD | 4.4 |
| 129300 | C20976 | Hs.110165 | ESTs; Highly similar to ribosomal protein | 5.7 |
| 129318 | N93155 | Hs.285976 | calmodulin 1 (phospholylase kinase; delta | 7.7 |
| 129319 | AA037467 | Hs.30340 | ESTs | 6 |
| 129351 | AA167268 | Hs.62349 | Human ras inhibitor mRNA; 3′ end | 9.3 |
| 129366 | H18027 | Hs.184697 | plexin C1 | 18.2 |
| 129383 | W92984 | Hs.288224 | ESTs | 5.9 |
| 129388 | AA151621 | Hs.110964 | ESTs | 4.1 |
| 129391 | T80814 | Hs.11101 | discs; large (Drosophila) homolog 3 (neur | 10.9 |
| 129404 | AA172056 | Hs.317584 | ESTs | 5.3 |
| 129406 | N23707 | Hs.111138 | KIAA0712 gene product | 4 |
| 129426 | AA412087 | Hs.111323 | EST; Highly similar to protein inhibitor o | 8 |
| 129453 | AA421213 | Hs.111632 | Lsm3 protein | 5.5 |
| 129513 | C00225 | Hs.306163 | ESTs; Weakly similar to fos39554_1 [H. s | 5.5 |
| 129519 | AA298786 | Hs.112242 | ESTs | 6.8 |
| 129606 | R21443 | Hs.166254 | heat shock 90 kD protein 1; alpha | 5 |
| 129622 | AA278243 | Hs.323949 | ESTs | 6.8 |
| 129626 | AA447410 | Hs.111334 | ESTs; Weakly similar to !!!! ALU SUBFA | 5.1 |
| 129627 | AA258308 | Hs.71968 | Homo sapiens mRNA; cDNA DKFZp564 | 5.3 |
| 129628 | U26727 | Hs.1174 | cyclin-dependent kinase inhibitor 2A (mel | 8.2 |
| 129642 | R50008 | Hs.11806 | 7-dehydrocholesterol reductase | 4.3 |
| 129663 | AA442768 | Hs.11866 | translocase of inner mitochondrial membr | 4.4 |
| 129665 | M88458 | Hs.118778 | KDEL (Lys-Asp-Glu-Leu) endoplasmic re | 4 |
| 129691 | X06700 | Hs.119571 | collagen; type III; alpha 1 (Ehlers-Danlos | 6 |
| 129783 | AA454618 | Hs.12479 | associated molecule with the SH3 domain | 6.4 |
| 129800 | AA252436 | Hs.12540 | lysophospholipase I | 7.7 |
| 129836 | AA452161 | Hs.206521 | YME1 (S. cerevisiae)-like 1 | 5 |
| 129850 | N20593 | Hs.288932 | GDP dissociation inhibitor 2 | 6.9 |
| 129869 | AA102520 | Hs.13015 | ESTs; Weakly similar to heat shock prote | 5 |
| 129896 | AA043021 | Hs.13225 | UDP-Gal: betaGlcNAc beta 1;4-galactosy | 6.6 |
| 129982 | M87789 | | immunoglobulin gamma 3 (Gm marker) | 4 |
| 129985 | AA450045 | Hs.140452 | cargo selection protein (mannose 6 phosp | 5.8 |
| 130029 | AA236412 | Hs.236510 | ESTs; Moderately similar to PFT27 [M. m | 5.6 |
| 130033 | M90696 | Hs.181301 | cathepsin S | 5.4 |
| 130036 | AA195260 | Hs.125849 | ESTs; Moderately similar to !!!! ALU SU | 7.4 |
| 130069 | AA055896 | Hs.146428 | collagen; type V; alpha 1 | 7.6 |
| 130077 | T24055 | Hs.91379 | ribosomal protein L26 | 4 |
| 130080 | X14850 | Hs.147097 | H2A histone family; member X | 12.1 |
| 130096 | AA223874 | Hs.197955 | KIAA0704 protein | 5 |
| 130114 | AA234717 | Hs.14992 | ESTs | 7.8 |
| 130125 | M36803 | Hs.1504 | hemopexin | 7.2 |
| 130135 | M61764 | Hs.21635 | tubulin; gamma 1 | 5.6 |
| 130170 | AA610070 | Hs.151469 | calcium/calmodulin-dependent serine pro | 7.5 |
| 130189 | D43947 | Hs.151761 | KIAA0100 gene product | 6.4 |
| 130208 | AA620556 | Hs.15250 | peroxisomal D3; D2-enoyl-CoA isomerase | 6.4 |
| 130211 | D50840 | Hs.23703 | UDP-glucose ceramide glucosyltransferas | 4.5 |
| 130235 | X14046 | Hs.153053 | CD37 antigen | 9.1 |
| 130276 | S75295 | Hs.169149 | karyopherin alpha 1 (importin alpha 5) | 8.6 |
| 130280 | L13738 | Hs.153937 | activated p21cdc42Hs kinase | 5 |
| 130313 | AA620323 | Hs.154320 | ubiquitin-activating enzyme E1C (homolo | 6.1 |
| 130314 | D86967 | Hs.154332 | KIAA0212 gene product | 10 |
| 130328 | AA135673 | Hs.154668 | KIAA0391 gene product | 6.1 |
| 130356 | X84373 | Hs.155017 | nuclear receptor interacting protein 1 | 10.6 |
| 130367 | Z38501 | Hs.8768 | ESTs; Weakly similar to !!!! ALU SUBFA | 8.3 |
| 130378 | T47333 | Hs.155188 | TATA box binding protein (TBP)-associa | 7.1 |
| 130384 | X66364 | Hs.166071 | cyclin-dependent kinase 5 | 5.6 |
| 130393 | D13630 | Hs.155291 | KIAA0005 gene product | 4.1 |
| 130399 | AA449417 | Hs.155356 | Homo sapiens mRNA for putative glucosy | 4.6 |
| 130407 | N29888 | Hs.155410 | ESTs | 7 |
| 130414 | M21121 | Hs.241392 | small inducible cytokine A5 (RANTES) | 4.1 |
| 130417 | U58522 | Hs.155485 | huntingtin-interacting protein 2 | 7.9 |
| 130421 | D21260 | Hs.178710 | clathrin; heavy polypeptide-like 2 | 4 |
| 130441 | U35835 | Hs.155637 | protein kinase; DNA-activated; catalytic p | 6.8 |
| 130455 | X17059 | Hs.155956 | N-acetyltransferase 1 (arylamine N-acetyl | 26.4 |
| 130498 | L38951 | Hs.180446 | karyopherin (importin) beta 1 | 4.8 |
| 130499 | AA416723 | Hs.158286 | Homo sapiens mRNA for KIAA0446 prot | 6.1 |
| 130511 | L32137 | Hs.1584 | cartilage oligomeric matrix protein (pseud | 8.3 |
| 130553 | AA430032 | Hs.252587 | pituitary tumor-transforming 1 | 7.5 |
| 130558 | H96654 | Hs.15984 | ESTs; Weakly similar to gene pp21 protei | 5.6 |
| 130568 | AA232535 | Hs.16085 | ESTs; Highly similar to CGI-13 protein [H | 4 |
| 130583 | W24957 | Hs.293907 | ESTs; Moderately similar to similar to C. e | 13.3 |
| 130585 | H66211 | Hs.16331 | ESTs | 10.1 |
| 130604 | X03635 | Hs.1657 | estrogen receptor 1 | 39.9 |
| 130614 | AA132007 | Hs.16697 | ESTs | 5.1 |
| 130619 | AA477739 | Hs.12532 | ESTs | 5.9 |
| 130622 | AA235247 | Hs.16846 | ESTs; Weakly similar to cytochrome P45 | 4.1 |
| 130625 | F03969 | Hs.260720 | matrix metalloproteinase 2 (gelatinase A; | 8.3 |
| 130627 | L23808 | Hs.1695 | matrix metalloproteinase 12 (macrophage | 10.3 |
| 130629 | M60346 | Hs.1697 | ATPase; H+ transporting; lysosomal (vacu | 7 |
| 130635 | M87503 | Hs.1706 | interferon-stimulated transcription factor | 5.5 |
| 130639 | D59711 | Hs.17132 | ESTs | 7.2 |
| 130677 | H17861 | Hs.17767 | ESTs | 13.5 |
| 130681 | D82808 | Hs.17820 | Rho-associated; coiled-coil containing pro | 6 |
| 130693 | AA487202 | Hs.17962 | ESTs | 6.1 |
| 130703 | N63295 | Hs.18103 | ESTs | 4.3 |
| 130706 | AA488843 | Hs.201673 | comichon-like | 4 |
| 130712 | AA292066 | Hs.279762 | adenylate cyclase 7 | 5.1 |
| 130714 | X92896 | Hs.18212 | DNA segment on chromosome X (unique) | 8.4 |
| 130715 | T98227 | Hs.171952 | occludin | 5.7 |
| 130744 | AA203527 | Hs.18747 | POP7 (processing of precursor; S. cerevis | 6.2 |
| 130747 | AA471293 | Hs.6879 | ESTs | 8.2 |
| 130751 | AA435633 | Hs.18879 | Homo sapiens clone 23965 mRNA sequen | 8.3 |
| 130796 | R39390 | Hs.19525 | ESTs | 4.5 |
| 130800 | AA223386 | Hs.19574 | ESTs; Weakly similar to katanin p80 subu | 7.7 |
| 130855 | AA425439 | Hs.143323 | putative DNA/chromatin binding motif | 4.3 |
| 130859 | AA287327 | Hs.20478 | ceroid-lipefuscinosis; neuronal 2; late infa | 9.8 |
| 130866 | M58028 | Hs.2055 | ubiquitin-activating enzyme E1 (A1S9T a | 4.3 |
| 130880 | D14678 | Hs.20830 | kinesin-like 2 | 4.5 |
| 130891 | D31891 | Hs.20991 | SET domain; bifurcated; 1 | 4 |
| 130905 | AA056489 | Hs.129998 | ESTs | 8.7 |
| 130913 | W03592 | Hs.21198 | translocase of outer mitochondrial membr | 20.9 |
| 130919 | AA291710 | Hs.21276 | collagen; type IV; alpha 3 (Goodpasture a | 9 |
| 130921 | AA074596 | Hs.194688 | bromodomain adjacent to zinc finger dom | 5.3 |
| 130944 | M97935 | Hs.21486 | signal transducer and activator of transcrip | 18.8 |
| 130974 | X57985 | Hs.2178 | H2B histone family; member Q | 13.4 |
| 130987 | R45698 | Hs.21893 | ESTs; Weakly similar to cAMP inducible | 8.5 |
| 130999 | N48963 | Hs.21992 | KIAA0689 protein | 7.2 |
| 131010 | AA435748 | Hs.169341 | ESTs; Weakly similar to phosphatidic acid | 5.2 |
| 131046 | X02530 | Hs.2248 | small inducible cytokine subfamily B (Cy | 10.1 |
| 131091 | T35341 | Hs.22880 | ESTs; Highly similar to dipeptidyl peptid | 6.3 |
| 131153 | H11760 | Hs.23606 | ESTs | 7.3 |
| 131185 | M25753 | Hs.23960 | cyclin B1 | 6.2 |
| 131200 | AA609427 | Hs.293732 | ESTs; Moderately similar to !!!! ALU SU | 4.3 |
| 131206 | AA044078 | Hs.24210 | ESTs | 5.5 |
| 131210 | AA430047 | Hs.95549 | ESTs | 7.1 |
| 131227 | AA429472 | Hs.236522 | DKFZP434P106 protein | 5.6 |
| 131244 | D38076 | Hs.24763 | RAN binding protein 1 | 5.5 |
| 131245 | AA620599 | Hs.24766 | DKFZP564E1962 protein | 6.7 |
| 131257 | AA256042 | Hs.24908 | ESTs | 5.8 |
| 131319 | U25997 | Hs.25590 | stanniocalcin | 8.9 |
| 131339 | AA463450 | Hs.25812 | Nijmegen breakage syndrome 1 (nibrin) | 6.5 |
| 131388 | R34531 | Hs.92200 | KIAA0480 gene product | 9.2 |
| 131410 | H84658 | Hs.279836 | ESTs | 12.1 |
| 131472 | AA608962 | Hs.27258 | calcyclin binding protein | 18.1 |
| 131475 | Z39053 | Hs.27263 | ESTs | 7.5 |
| 131501 | AA121127 | Hs.8207 | H3 histone; family 3A | 5.5 |
| 131514 | X02152 | Hs.2795 | lactate dehydrogenase A | 5.1 |
| 131524 | N39152 | Hs.301804 | ESTs | 4.3 |
| 131528 | D60856 | Hs.28309 | UDP-glucose dehydrogenase | 8.4 |
| 131544 | N33236 | Hs.28555 | ESTs; Weakly similar to B0511.8 [C. eleg | 5.6 |
| 131557 | D30946 | Hs.28707 | signal sequence receptor; gamma (transloc | 8.7 |
| 131562 | U90551 | Hs.28777 | H2A histone family; member L | 18.8 |
| 131564 | AA491465 | Hs.28792 | ESTs | 11.8 |
| 131586 | AA235385 | Hs.26966 | ESTs; Moderately similar to alternatively | 4.7 |
| 131587 | M15182 | Hs.183868 | glucuronidase; beta | 5.2 |
| 131589 | U52100 | Hs.29191 | epithelial membrane protein 2 | 4.4 |
| 131615 | D14533 | Hs.192803 | xeroderma pigmentosum; complementatio | 4.6 |
| 131664 | AA136126 | Hs.30327 | mitogen-activated protein kinase-activated | 4.3 |
| 131679 | AA136660 | Hs.30579 | ESTs | 9.4 |
| 131684 | U26174 | Hs.3066 | granzyme K (serine protease; granzyme 3 | 9.7 |
| 131687 | L11066 | Hs.3069 | heat shock 70 kD protein 9B (mortalin-2) | 6.2 |
| 131689 | AA599653 | Hs.30696 | transcription factor-like 5 (basic helix-loo | 8.3 |
| 131693 | W60913 | Hs.110796 | ESTs; Weakly similar to cDNA EST yk45 | 9 |
| 131710 | AA233225 | Hs.30985 | MRS1 protein | 5.2 |
| 131716 | D49738 | Hs.31053 | cytoskeleton-associated protein 1 | 6.6 |
| 131742 | D31352 | Hs.31433 | ESTs | 11 |
| 131762 | H46831 | Hs.107767 | ESTs; Moderately similar to CaM-KII inh | 4.9 |
| 131781 | AA460450 | Hs.31989 | DKFZP586G1722 protein | 9.2 |
| 131795 | N32724 | Hs.32317 | Sox-like transcriptional factor | 4.5 |
| 131809 | L76517 | Hs.3260 | presenilin 1 (Alzheimer disease 3) | 5.4 |
| 131814 | AA437226 | Hs.157 | interleukin 10 receptor; alpha | 4 |
| 131838 | AA091932 | Hs.180628 | dynamin-like protein | 6.7 |
| 131877 | J04088 | Hs.156346 | topoisomerase (DNA) II alpha (170 kD) | 5 |
| 131885 | AA044095 | Hs.3402 | ESTs | 11.1 |
| 131891 | AA158258 | Hs.30376 | heterogeneous nuclear protein similar to r | 5.6 |
| 131925 | AA248470 | Hs.183180 | ESTs; Weakly similar to RING finger pro | 4.5 |
| 131930 | AA205460 | Hs.69476 | ESTs | 14.3 |
| 131941 | D62657 | Hs.35086 | ubiquitin-specific protease 1 | 6.2 |
| 131965 | W90146 | Hs.35962 | ESTs | 6.3 |
| 131970 | D86960 | Hs.3610 | KIAA0205 gene product | 4.2 |
| 131971 | R70167 | Hs.154938 | ESTs | 4.3 |
| 131974 | AA410424 | Hs.268122 | Homo sapiens mRNA; cDNA DKFZp586 | 4.6 |
| 131977 | F09788 | Hs.3622 | procollagen-proline; 2-oxoglutarate 4-diox | 6.4 |
| 131994 | AA479515 | Hs.279882 | Human DNA sequence from clone 703H1 | 12 |
| 131997 | D82399 | Hs.136644 | Homo sapiens clone 23714 mRNA sequen | 10 |
| 132017 | W67251 | Hs.267659 | Homo sapiens vav 3 oncogene (VAV3) m | 4.7 |
| 132021 | T68246 | Hs.306079 | chaperonin containing TCP1; subunit 5 (e | 5.2 |
| 132065 | D82226 | Hs.211594 | proteasome (prosome; macropain) 26S sub | 8.5 |
| 132085 | D44466 | Hs.3887 | proteasome (prosome; macropain) 26S sub | 13.5 |
| 132089 | AA131971 | Hs.39122 | ESTs | 4.8 |
| 132109 | AA599801 | Hs.40098 | ESTs | 6.2 |
| 132143 | AA257056 | Hs.7972 | KIAA0871 protein | 14.6 |
| 132149 | T10822 | Hs.324743 | ESTs | 5.3 |
| 132153 | N90141 | Hs.41066 | ESTs; Moderately similar to ELONGATI | 9.2 |
| 132160 | AA281770 | Hs.295923 | seven in absentia (Drosophila) homolog 1 | 5.5 |
| 132164 | U84573 | Hs.41270 | procollagen-lysine; 2-oxoglutarate 5-dioxy | 8.1 |
| 132180 | AA405569 | Hs.418 | fibroblast activation protein; alpha; sepras | 15.4 |
| 132183 | L19183 | Hs.199695 | hypothetical protein | 12.2 |
| 132225 | AA128980 | | ESTs | 5.6 |
| 132227 | AA412620 | Hs.4248 | ESTs | 6.7 |
| 132235 | F09058 | Hs.42656 | ESTs | 6.2 |
| 132256 | AA608856 | Hs.431 | murine leukemia viral (bmi-1) oncogene h | 6 |
| 132298 | N41849 | Hs.7120 | Homo sapiens cytokine receptor related p | 5.6 |
| 132314 | AA285290 | Hs.44499 | small EDRK-rich factor 2 | 6.8 |
| 132325 | N37065 | Hs.44856 | ESTs | 4.7 |
| 132384 | AA479933 | Hs.46967 | Human DNA sequence from clone 167A1 | 4.2 |
| 132387 | R70914 | Hs.281434 | heat shock 70 kD protein 1 | 9.1 |
| 132393 | W85888 | Hs.47334 | ESTs; Moderately similar to !!!! ALU SU | 4 |
| 132406 | F09979 | Hs.4774 | ESTs | 15 |
| 132407 | AA431459 | Hs.47783 | ESTs | 8 |
| 132413 | AA132969 | Hs.260116 | KIAA1104 protein | 4 |
| 132446 | AA426218 | Hs.48764 | ESTs | 5.3 |
| 132465 | AA047896 | Hs.49169 | ESTs | 15.4 |
| 132482 | AA429478 | Hs.238126 | ESTs; Highly similar to CGI-49 protein [H | 9 |
| 132492 | T03749 | Hs.4990 | KIAA1089 protein | 8.5 |
| 132528 | AA283006 | Hs.50758 | chromosome-associated polypeptide C | 4.3 |
| 132540 | AA488987 | Hs.5097 | synaptogyrin 2 | 9.8 |
| 132543 | AA417152 | Hs.5101 | protein regulator of cytokinesis 1 | 10.1 |
| 132580 | L37042 | Hs.283738 | casein kinase 1; alpha 1 | 5.9 |
| 132586 | AA412452 | Hs.52515 | DKFZP434N024 protein | 4.2 |
| 132608 | AA199588 | Hs.5321 | ARP3 (actin-related protein 3; yeast) hom | 4.2 |
| 132616 | AA386264 | Hs.283558 | isocitrate dehydrogenase 2 (NADP+); mit | 5.2 |
| 132617 | AA171913 | Hs.5338 | carbonic anhydrase XII | 10.1 |
| 132618 | AA253330 | Hs.279916 | adaptor-related protein complex 1; gamma | 4.8 |
| 132640 | U33821 | Hs.5437 | Tax1 (human T-cell leukemia virus type I | 5.7 |
| 132668 | AA453614 | Hs.5460 | KIAA0776 protein | 4.4 |
| 132694 | M60830 | Hs.5509 | ecotropic viral integration site 2B | 15.6 |
| 132700 | N47109 | Hs.5521 | ESTs | 7 |
| 132724 | AA417962 | Hs.55498 | geranylgeranyl diphosphate synthase 1 | 5.6 |
| 132738 | W42674 | Hs.264636 | ESTs; Moderately similar to neuronal thre | 4.9 |
| 132742 | AA490862 | Hs.292812 | ESTs; Weakly similar to C43H8.1 [C. eleg | 7.9 |
| 132744 | X54326 | Hs.55921 | glutamyl-prolyl-tRNA synthetase | 4.1 |
| 132795 | H99152 | Hs.57079 | ESTs | 8 |
| 132807 | AA331777 | Hs.57301 | mutL (E. coli) homolog 1 (colon cancer; n | 8 |
| 132811 | U25435 | Hs.57419 | transcriptional repressor | 4 |
| 132817 | AB004884 | Hs.57553 | tousled-like kinase 2 | 6.5 |
| 132840 | N23817 | Hs.5807 | Homo sapiens clone 23675 mRNA sequen | 5.6 |
| 132845 | D62588 | Hs.5813 | ESTs | 12.4 |
| 132847 | T48195 | Hs.58189 | eukalyotic translation initiation factor 3; s | 7 |
| 132856 | W79865 | Hs.58367 | glypican 4 | 6.2 |
| 132869 | N26855 | Hs.203961 | ESTs | 6.5 |
| 132874 | AA425776 | Hs.58609 | ESTs | 5.6 |
| 132880 | AA444369 | Hs.177537 | ESTs | 7.2 |
| 132894 | D82422 | Hs.5944 | ESTs | 7.5 |
| 132900 | N56451 | Hs.5978 | LIM domain only 7 | 4.4 |
| 132903 | AA235404 | Hs.5985 | Homo sapiens clone 25186 mRNA sequen | 9.1 |
| 132904 | X83618 | Hs.59889 | 3-hydroxy-3-methylglutaryl-Coenzyme A | 10.7 |
| 132906 | AA142857 | Hs.234896 | ESTs; Highly similar to geminin [H. sapie | 10.2 |
| 132914 | AA496037 | Hs.60293 | ESTs | 4.7 |
| 132918 | AA252605 | Hs.6051 | KIAA0616 protein | 7.1 |
| 132936 | AB002305 | Hs.6111 | KIAA0307 gene product | 8.3 |
| 132951 | U04209 | Hs.61418 | microfibrillar-associated protein 1 | 4.3 |
| 132957 | AA234791 | Hs.61469 | Human gene from PAC 753P9; chromoso | 13.2 |
| 132959 | AA028103 | Hs.61472 | ESTs; Weakly similar to unknown [S. cere | 18.9 |
| 132968 | N77151 | Hs.61638 | myosin X | 5.8 |
| 132984 | H80409 | Hs.62112 | zinc finger protein 207 | 4.3 |
| 132990 | AA458761 | Hs.18387 | transcription factor AP-2 alpha (activating | 4.2 |
| 132994 | AA505133 | Hs.279905 | solute carrier family 2 (facilitated glucose | 26.4 |
| 132998 | Y00062 | Hs.170121 | protein tyrosine phosphatase; receptor typ | 4.4 |
| 133002 | AF006082 | Hs.42915 | ARP2 (actin-related protein 2; yeast) hom | 4.7 |
| 133005 | C21400 | Hs.278605 | KIAA0970 protein | 6.6 |
| 133015 | AA047036 | Hs.246315 | ESTs | 7.9 |
| 133016 | W81298 | Hs.6289 | growth factor receptor-bound protein 2 | 5.2 |
| 133039 | X62055 | Hs.63489 | protein tyrosine phosphatase; non-recepto | 4 |
| 133050 | S67325 | Hs.63788 | propionyl Coenzyme A carboxylase; beta | 5.2 |
| 133056 | AA071387 | Hs.6396 | jumping translocation breakpoint | 5 |
| 133062 | R33663 | Hs.64056 | ESTs | 5.4 |
| 133083 | N70633 | Hs.6456 | chaperonin containing TCP1; subunit 2 (b | 6 |
| 133091 | AA122147 | Hs.64691 | K1AA0483 protein | 5 |
| 133093 | AA598749 | Hs.285996 | ESTs | 5.6 |
| 133124 | AA156049 | Hs.267923 | ESTs | 4.1 |
| 133126 | D16469 | Hs.6551 | ATPase; H+ transporting; lysosomal (vacu | 6.2 |
| 133196 | R37367 | Hs.6727 | Ras-GTPase activating protein SH3 doma | 5.1 |
| 133214 | Y10659 | Hs.285115 | interleukin 13 receptor; alpha 1 | 6.2 |
| 133225 | Z41415 | Hs.6823 | ESTs; Weakly similar to intrinsic factor-B | 8.3 |
| 133228 | N90029 | Hs.6831 | Homo sapiens clone 1400 unknown prote | 4.7 |
| 133239 | AA059405 | Hs.179882 | Homo sapiens clone 24655 mRNA sequen | 5.5 |
| 133240 | D31161 | Hs.242894 | ESTs | 9 |
| 133257 | AF006086 | Hs.6895 | actin related protein 2/3 complex; subunit | 7.7 |
| 133264 | W72187 | Hs.69192 | ESTs; Weakly similar to cDNA EST yk37 | 6.7 |
| 133274 | AA488886 | Hs.6949 | ESTs | 4.2 |
| 133281 | AA421079 | Hs.69594 | ESTs; Weakly similar to Sox-like transcri | 4.9 |
| 133283 | AA410507 | Hs.6968 | ESTs | 4.3 |
| 133287 | L15702 | Hs.69771 | B-factor; properdin | 9.3 |
| 133294 | R79723 | Hs.69997 | zinc finger protein 238 | 30.4 |
| 133297 | AA600057 | Hs.70266 | KIAA0905 protein | 10.4 |
| 133318 | AA256168 | Hs.152316 | ESTs | 8.5 |
| 133362 | H06195 | Hs.7194 | ESTs; Highly similar to CGI-59 protein [H | 14 |
| 133370 | AA156897 | Hs.72157 | DKFZP564I1922 protein | 5 |
| 133391 | X57579 | Hs.727 | inhibin; beta A (activin A; activin AB alp | 13.9 |
| 133395 | AA491296 | Hs.72805 | ESTs | 4.3 |
| 133422 | N79516 | Hs.73287 | ESTs; Weakly similar to eyelid [D. melano | 4.5 |
| 133431 | AA255438 | Hs.7358 | Homo sapiens mRNA; cDNA DKFZp566 | 8 |
| 133435 | T23983 | Hs.323966 | ESTs | 5 |
| 133449 | AA094989 | Hs.7381 | voltage-dependent anion channel 3 | 8.7 |
| 133468 | X03068 | Hs.73931 | major histocompatibility complex; class II | 5 |
| 133484 | X78710 | Hs.211581 | metal-regulatory transcription factor 1 | 5.3 |
| 133506 | AA316868 | Hs.74346 | ESTs; Weakly similar to 140G11.h [D. me | 6.8 |
| 133517 | X52947 | Hs.74471 | gap junction protein; alpha 1; 43 kD (conn | 5.7 |
| 133551 | D63480 | Hs.278634 | KIAA0146 protein | 4.8 |
| 133569 | AA313977 | Hs.172772 | transcription elongation factor B (SIII); po | 9.5 |
| 133572 | W94333 | Hs.279915 | translocase of inner mitochondrial membr | 5 |
| 133577 | F03717 | Hs.75063 | human immunodeficiency virus type I enh | 7.4 |
| 133589 | L37368 | Hs.75104 | RNA-binding protein S1; serine-rich dom | 5 |
| 133608 | D13315 | Hs.75207 | glyoxalase I | 4.2 |
| 133617 | AA148318 | Hs.75249 | KIAA0069 protein | 4.5 |
| 133627 | U09587 | Hs.75280 | glycyl-tRNA synthetase | 10 |
| 133633 | D21262 | Hs.75337 | nucleolar phosphoprotein p130 | 4.5 |
| 133634 | U24166 | Hs.234279 | microtubule-associated protein; RP/EB fa | 15.2 |
| 133640 | D83004 | Hs.75355 | ubiquitin-conjugating enzyme E2N (homo | 9.1 |
| 133644 | D89077 | Hs.75367 | Src-like-adapter | 6.4 |
| 133649 | AA479139 | Hs.75393 | acid phosphatase 1; soluble | 4.8 |
| 133652 | AA287383 | Hs.7540 | ESTs | 4.2 |
| 133674 | AA458946 | Hs.75497 | ESTs | 4.3 |
| 133700 | K01396 | Hs.297681 | protease inhibitor 1 (anti-elastase); alpha- | 8.3 |
| 133705 | N21648 | Hs.75659 | MpV17 transgene; murine homolog; glom | 4.6 |
| 133716 | Y00282 | Hs.75722 | ribophorin II | 7.5 |
| 133720 | L27841 | Hs.75737 | pericentriolar material 1 | 9.4 |
| 133752 | U49278 | Hs.75875 | ubiquitin-conjugating enzyme E2 variant | 4.5 |
| 133765 | D21255 | Hs.75929 | cadherin 11 (OB-cadherin; osteoblast) | 6.4 |
| 133772 | W73693 | Hs.76038 | isopentenyl-diphosphate delta isomerase | 7.9 |
| 133774 | Z23090 | Hs.76067 | heat shock 27 kD protein 1 | 4.1 |
| 133776 | J03473 | Hs.177766 | ADP-ribosyltransferase (NAD+; poly (AD | 13 |
| 133784 | AA214305 | Hs.301064 | ESTs | 5.2 |
| 133814 | M33882 | Hs.76391 | myxovirus (influenza) resistance 1; homol | 11.7 |
| 133829 | AA453783 | Hs.76550 | Homo sapiens mRNA; cDNA DKFZp564 | 9.4 |
| 133834 | AA147510 | Hs.288660 | serine protease; umbilical endothelium | 4.8 |
| 133839 | M59815 | Hs.170250 | complement component 4A | 6.7 |
| 133842 | U73477 | Hs.285013 | putative human HLA class II associated p | 7.1 |
| 133845 | T68510 | Hs.76704 | ESTs | 6.3 |
| 133859 | U86782 | Hs.178761 | 26S proteasome-associated pad1 homolog | 13.7 |
| 133867 | D43948 | Hs.76989 | KIAA0097 gene product | 4.1 |
| 133868 | U58090 | Hs.183874 | cullin 4A | 4 |
| 133871 | AA454597 | Hs.182793 | ESTs | 4.7 |
| 133893 | X01060 | Hs.77356 | transferrin receptor (p90; CD71) | 8.3 |
| 133914 | N32811 | Hs.77542 | ESTs | 5 |
| 133918 | W72783 | Hs.58382 | ESTs; Weakly similar to C13F10.5 [C. ele | 4.5 |
| 133944 | AA045870 | Hs.7780 | Homo sapiens mRNA; cDNA DKFZp564 | 6.3 |
| 133946 | AA156565 | Hs.173878 | 4-nitrophenylphosphatase domain and non | 6.4 |
| 133963 | L34587 | Hs.184693 | transcription elongation factor B (SIII); po | 6.3 |
| 133980 | D00760 | Hs.250811 | proteasome (prosome; macropain) subunit | 11.9 |
| 133990 | C02374 | Hs.7822 | Homo sapiens mRNA; cDNA DKFZp564 | 8.2 |
| 133999 | M28213 | Hs.78305 | RAB2; member RAS oncogene family | 5.2 |
| 134030 | J03077 | Hs.78575 | prosaposin (variant Gaucher disease and v | 4.6 |
| 134032 | Z81326 | Hs.78589 | protease inhibitor 12 (neuroserpin) | 6.5 |
| 134045 | S82470 | Hs.78768 | BB1 | 11.9 |
| 134046 | D28473 | Hs.172801 | isoleucine-tRNA synthetase | 5.2 |
| 134064 | D87685 | Hs.78893 | KIAA0244 protein | 7.3 |
| 134070 | H98621 | Hs.78946 | cullin 3 | 4.7 |
| 134087 | U51166 | Hs.173824 | thymine-DNA glycosylase | 7 |
| 134090 | M22382 | Hs.79037 | heat shock 60 kD protein 1 (chaperonin) | 4.5 |
| 134098 | X06323 | Hs.79086 | ribosomal protein; mitochondrial; L3 | 9.4 |
| 134110 | U41060 | Hs.79136 | LIV-1 protein; estrogen regulated | 4.4 |
| 134132 | U32519 | Hs.220689 | Ras-GTPase-activating protein SH3-doma | 6.6 |
| 134168 | AA398908 | Hs.181634 | Human Chromosome 16 BAC clone CIT9 | 8.6 |
| 134170 | M63138 | Hs.79572 | cathepsin D (lysosomal aspartyl protease) | 9.3 |
| 134208 | U88871 | Hs.79993 | peroxisomal biogenesis factor 7 | 6.3 |
| 134258 | L28010 | Hs.808 | heterogeneous nuclear ribonucleoprotein F | 4.3 |
| 134288 | AA430008 | Hs.8117 | ESTs | 6.9 |
| 134310 | AA313414 | Hs.8148 | Homo sapiens clone 24856 mRNA sequen | 7.4 |
| 134326 | U16306 | Hs.81800 | chondroitin sulfate proteoglycan 2 (versic | 6.1 |
| 134329 | D38551 | Hs.81848 | RAD21 (S. pombe) homolog | 8.6 |
| 134331 | AA452020 | Hs.111222 | ESTs; Weakly similar to CGI-128 protein | 6.1 |
| 134351 | R82074 | Hs.82109 | syndecan 1 | 4.4 |
| 134357 | L43575 | Hs.82171 | Human clone 191B7 placenta expressed m | 6.6 |
| 134363 | M37033 | Hs.82212 | CD53 antigen | 5.3 |
| 134367 | X54199 | Hs.82285 | phosphoribosylglycinamide formyltransfe | 4.8 |
| 134374 | D62633 | Hs.8236 | ESTs | 15.2 |
| 134375 | AA412720 | Hs.82389 | ESTs; Highly similar to CGI-118 protein | 7.2 |
| 134376 | X02874 | Hs.82396 | 2′; 5′-oligoadenylate synthetase 1 | 6.4 |
| 134381 | U56637 | Hs.184270 | capping protein (actin filament) muscle Z- | 4 |
| 134388 | M15841 | Hs.82575 | small nuclear ribonucleoprotein polypepti | 5.7 |
| 134395 | L09717 | Hs.8262 | lysosomal-associated membrane protein 2 | 6.9 |
| 134399 | H99801 | Hs.82689 | tumor rejection antigen (gp96) 1 | 4.5 |
| 134401 | AA243746 | Hs.211577 | kinectin 1 (kinesin receptor) | 11.2 |
| 134405 | J04177 | Hs.82772 | collagen; type XI; alpha 1 | 15.3 |
| 134415 | AA329274 | Hs.82911 | protein tyrosine phosphatase type IVA; m | 4.1 |
| 134417 | D87969 | Hs.82921 | solute carrier family 35 (CMP-sialic acid t | 4.2 |
| 134419 | L08044 | Hs.82961 | trefoil factor 3 (intestinal) | 5.9 |
| 134421 | AA122386 | Hs.82985 | collagen; type V; alpha 2 | 5.8 |
| 134423 | W96151 | Hs.83006 | ESTs; Highly similar to CGI-139 protein | 4.4 |
| 134438 | AA449984 | Hs.246857 | ESTs; Highly similar to proteine kinase JN | 7 |
| 134446 | T25732 | Hs.83419 | KIAA0252 protein | 4.6 |
| 134453 | X70683 | Hs.83484 | SRY (sex determining region Y)-box 4 | 5.1 |
| 134470 | X54942 | Hs.83758 | CDC28 protein kinase 2 | 20.3 |
| 134487 | R38185 | Hs.83954 | Homo sapiens unknown mRNA | 5 |
| 134495 | D63477 | Hs.84087 | KIAA0143 protein | 16.1 |
| 134498 | M63180 | Hs.84131 | threonyl-tRNA synthetase | 6.1 |
| 134506 | U45328 | Hs.84285 | ubiquitin-conjugating enzyme E2I (homol | 4.6 |
| 134529 | H24460 | Hs.848 | FK506-binding protein 4 (59 kD) | 6.2 |
| 134570 | U66615 | Hs.172280 | SWI/SNF related; matrix associated; actin | 4.8 |
| 134582 | AA234966 | Hs.86041 | CGG triplet repeat binding protein 1 | 4.7 |
| 134600 | R68884 | Hs.86347 | ESTs; Weakly similar to predicted using G | 5.8 |
| 134623 | X74496 | Hs.86978 | prolyl endopeptidase | 4.5 |
| 134654 | W23625 | Hs.8739 | ESTs; Weakly similar to ORF YGR200c [ | 13.7 |
| 134655 | AA454070 | Hs.123090 | ESTs | 5.8 |
| 134675 | AA250745 | Hs.87773 | protein kinase; cAMP-dependent; catalyti | 8.9 |
| 134711 | X04011 | Hs.88974 | cytochrome b-245; beta polypeptide (chro | 6.8 |
| 134714 | U89922 | Hs.890 | lymphotoxin beta (TNF superfamily; mem | 35.7 |
| 134722 | W47183 | Hs.284226 | ESTs; Weakly similar to neural F box pro | 8.1 |
| 134776 | J05582 | Hs.89603 | mucin 1; transmembrane | 6.2 |
| 134806 | Z49099 | Hs.89718 | spermine synthase | 4.2 |
| 134810 | M27394 | Hs.89751 | membrane-spanning 4-domains; subfamily | 7 |
| 134840 | U51477 | Hs.89981 | diacylglycerol kinase; zeta (104 kD) | 4.1 |
| 134843 | H60595 | Hs.90061 | progesterone binding protein | 4.7 |
| 134853 | D82348 | Hs.90280 | 5-aminoimidazole-4-carboxamide ribonuc | 10.2 |
| 134866 | U84011 | Hs.904 | amylo-1; 6-glucosidase; 4-alpha-glucanotr | 12.1 |
| 134868 | Z39762 | Hs.90419 | KIAA0882 protein | 6 |
| 134885 | N27670 | Hs.9071 | progesterone membrane binding protein | 5 |
| 134982 | N46086 | Hs.92308 | ESTs | 4.1 |
| 134989 | AA236324 | Hs.92381 | Homo sapiens mRNA; chromosome 1 spe | 16.8 |
| 134992 | H05625 | Hs.5831 | ESTs | 4 |
| 134993 | AA282343 | Hs.301005 | purine-rich element binding protein B | 4.4 |
| 135010 | D59675 | Hs.92927 | ESTs | 7 |
| 135015 | U54999 | Hs.278338 | LGN protein | 4.8 |
| 135029 | AA224180 | | ESTs; Moderately similar to 17-beta-hydr | 13.6 |
| 135032 | AA243497 | Hs.173685 | Human DNA sequence from clone 30M3 | 4 |
| 135037 | U77948 | Hs.278589 | general transcription factor II; i | 8 |
| 135059 | AA598449 | Hs.93832 | Homo sapiens clone 24483 unknown mRN | 5.4 |
| 135071 | L08069 | Hs.94 | heat shock protein; DNAJ-like 2 | 9.3 |
| 135083 | AA495950 | Hs.94262 | ESTs | 6.7 |
| 135117 | W52493 | Hs.94694 | Homo sapiens clone 24837 mRNA sequen | 10.2 |
| 135144 | AA044842 | Hs.95260 | Homo sapiens mRNA; cDNA DKFZp586 | 6.6 |
| 135154 | AA126433 | Hs.267812 | sorting nexin 4 | 7.4 |
| 135218 | D31157 | Hs.324277 | ESTs; Weakly similar to growth factor-res | 6.2 |
| 135237 | AA454930 | Hs.9691 | ESTs | 19.5 |
| 135243 | AA215333 | Hs.97101 | putative G protein-coupled receptor | 8.8 |
| 135335 | H20989 | Hs.198281 | pyruvate kinase; muscle | 12.4 |
| 135349 | D83174 | Hs.9930 | collagen-binding protein 2 (colligen 2) | 5.5 |
| 135367 | AA480109 | Hs.9963 | TYRO protein tyrosine kinase binding pro | 5.4 |
| 135389 | U05237 | Hs.99872 | fetal Alzheimer antigen | 7.8 |
| 135400 | M23263 | Hs.99915 | androgen receptor (dihydrotestosterone re | 9.1 |
| 135411 | L10333 | Hs.99947 | reticulon 1 | 5.3 |
| 300019 | M97935 | | AFFX control: STAT1 | 8.3 |
| 300021 | M97935 | | AFFX control: STAT1 | 7 |
| 300022 | M97935 | | AFFX control: STAT1 | 14 |
| 300089 | AI199738 | Hs.208275 | ESTs; Weakly similar to !!!! ALU CLASS | 9.1 |
| 300107 | AI694585 | Hs.270464 | ESTs; Weakly similar to !!!! ALU CLASS | 7.4 |
| 300254 | AW079607 | Hs.188417 | ESTs; Weakly similar to ZnT-3 [H. sapien | 30.1 |
| 300328 | AW015860 | Hs.224623 | ESTs | 11.9 |
| 300549 | AA699328 | Hs.298119 | ESTs | 5.5 |
| 300711 | AI492179 | Hs.166244 | ESTs; Weakly similar to cDNA EST yk40 | 11 |
| 300921 | AW293224 | Hs.232165 | ESTs | 11 |
| 301124 | T79326 | Hs.298262 | ESTs; Weakly similar to dJ88J8.1 [H. sapi | 8.8 |
| 301165 | N85789 | Hs.150186 | ESTs; Weakly similar to PTERIN-4-ALP | 6 |
| 301576 | AI682905 | Hs.270431 | ESTs; Weakly similar to !!!! ALU SUBFA | 4.7 |
| 301604 | AA373124 | Hs.24809 | ESTs; Weakly similar to C17G10.1 [C. ele | 8 |
| 301704 | AA526313 | Hs.293691 | ESTs | 4.2 |
| 301782 | N99399 | Hs.143046 | EST cluster (not in UniGene) with exon h | 18 |
| 301884 | AA312082 | Hs.105445 | GDNF family receptor alpha 1 | 20.7 |
| 301936 | NM_004694 | Hs.114924 | EST cluster (not in UniGene) with exon h | 11.6 |
| 302002 | AF013956 | Hs.5637 | chromobox homolog 4 (Drosophila Pc cla | 9.2 |
| 302032 | NM_001992 | Hs.128087 | EST cluster (not in UniGene) with exon h | 4.3 |
| 302067 | H05698 | Hs.222399 | ESTs; Weakly similar to protein-tyrosine | 7.8 |
| 302145 | NM_003613 | Hs.151407 | EST cluster (not in UniGene) with exon h | 15.1 |
| 302236 | AI128606 | Hs.6557 | zinc finger protein 161 | 25.8 |
| 302276 | NM_004448 | Hs.323910 | EST cluster (not in UniGene) with exon h | 21.6 |
| 302290 | AL117607 | Hs.175563 | Homo sapiens mRNA; cDNA DKFZp564 | 41.4 |
| 302326 | NM_004271 | Hs.184018 | EST cluster (not in UniGene) with exon h | 8.9 |
| 302342 | AB023141 | Hs.190386 | KIAA0924 protein | 5.4 |
| 302372 | AL117406 | Hs.200102 | Homo sapiens mRNA; cDNA DKFZp434 | 8.9 |
| 302422 | AB021227 | Hs.3743 | matrix metalloproteinase 24 (membrane-in | 5.2 |
| 302431 | AF129530 | Hs.226434 | EST cluster (not in UniGene) with exon h | 5.3 |
| 302501 | AF022726 | Hs.251446 | EST cluster (not in UniGene) with exon h | 9.9 |
| 302505 | AL049650 | Hs.247874 | multiple UniGene matches | 4.3 |
| 302533 | L36149 | Hs.248116 | chemokine (C motif) XC receptor 1 | 4.9 |
| 302638 | AA463798 | Hs.102696 | ESTs; Weakly similar to C11D2.4 [C. eleg | 5.3 |
| 302656 | AW293005 | Hs.70704 | ESTs | 8.4 |
| 302792 | AA343696 | Hs.46821 | ESTs; Weakly similar to putative [H. sapie | 4.5 |
| 302820 | X04588 | Hs.85844 | EST cluster (not in UniGene) with exon h | 6.8 |
| 302838 | U66049 | Hs.82171 | EST cluster (not in UniGene) with exon h | 8.4 |
| 302892 | N58545 | Hs.42346 | histone deacetylase 3 | 22.8 |
| 302977 | AW263124 | Hs.315111 | EST cluster (not in UniGene) with exon h | 6.8 |
| 302989 | N46406 | Hs.84700 | EST cluster (not in UniGene) with exon h | 8.9 |
| 303007 | AA478876 | Hs.317714 | pallid (mouse) homolog; pallidin | 10.1 |
| 303052 | AF140242 | Hs.279926 | EST cluster (not in UniGene) with exon h | 24.4 |
| 303131 | AW081061 | Hs.103180 | actin-like 6 | 6.3 |
| 303132 | AI929819 | Hs.4055 | ESTs | 17.7 |
| 303153 | U09759 | Hs.246857 | mitogen-activated protein kinase 9 | 11.4 |
| 303387 | AA908797 | Hs.180799 | ESTs | 15.8 |
| 303499 | AI815990 | Hs.293515 | ESTs | 7.2 |
| 303502 | AA488528 | | EST cluster (not in UniGene) with exon h | 5.3 |
| 303576 | T07216 | Hs.301226 | EST cluster (not in UniGene) with exon h | 16.2 |
| 303620 | AA397546 | Hs.119151 | ESTs | 8.9 |
| 303634 | AI953377 | Hs.28444 | ESTs; Weakly similar to predicted using G | 12 |
| 303642 | AW299459 | Hs.111977 | EST cluster (not in UniGene) with exon h | 4.2 |
| 303654 | AA436942 | Hs.288529 | ESTs | 8.4 |
| 303733 | AW502498 | Hs.15220 | ESTs; Weakly similar to zinc finger prote | 5.2 |
| 303780 | AI424014 | Hs.18995 | ESTs; Moderately similar to KIAA0456 p | 28.4 |
| 303792 | C75094 | Hs.199839 | ESTs; Highly similar to NG22 [H. sapiens | 4.4 |
| 303842 | AI337304 | Hs.126268 | ESTs; Weakly similar to similar to PDZ d | 8.1 |
| 303951 | AW475081 | Hs.172928 | collagen; type I; alpha 1 | 7.5 |
| 304465 | AA421948 | | EST singleton (not in UniGene) with exon | 6.5 |
| 304507 | AA456426 | | EST | 5.4 |
| 304591 | AA505702 | | EST singleton (not in UniGene) with exon | 9.8 |
| 304601 | AA507875 | | EST singleton (not in UniGene) with exon | 7.5 |
| 304659 | AA533185 | | EST singleton (not in UniGene) with exon | 7 |
| 305040 | AA630582 | Hs.169476 | glyceraldehyde-3-phosphate dehydrogena | 12.4 |
| 305134 | AA653159 | Hs.179661 | EST singleton (not in UniGene) with exon | 8.7 |
| 305415 | AA725116 | Hs.78465 | EST singleton (not in UniGene) with exon | 5.3 |
| 305453 | AA738110 | | EST singleton (not in UniGene) with exon | 4.1 |
| 305898 | AA872838 | | keratin 8 | 7.7 |
| 305913 | AA876109 | | EST singleton (not in UniGene) with exon | 6.3 |
| 305950 | AA884479 | | EST singleton (not in UniGene) with exon | 5.6 |
| 306004 | AA889992 | Hs.2186 | EST singleton (not in UniGene) with exon | 13.2 |
| 306009 | AA894560 | Hs.283370 | EST singleton (not in UniGene) with exon | 4.4 |
| 306060 | AA906161 | Hs.76277 | EST singleton (not in UniGene) with exon | 4.6 |
| 306398 | AA970548 | Hs.297681 | EST singleton (not in UniGene) with exon | 7.6 |
| 306505 | AA987722 | Hs.172928 | EST singleton (not in UniGene) with exon | 19.7 |
| 306576 | AA995761 | Hs.276092 | EST singleton (not in UniGene) with exon | 5.5 |
| 307117 | AI184111 | Hs.76067 | heat shock 27 kD protein 1 | 7.7 |
| 307138 | AI185516 | Hs.172928 | collagen; type I; alpha 1 | 8.8 |
| 307187 | AI190870 | Hs.276417 | EST singleton (not in UniGene) with exon | 4.1 |
| 307542 | AI280859 | Hs.62954 | EST singleton (not in UniGene) with exon | 6 |
| 307554 | AI281603 | Hs.172928 | EST singleton (not in UniGene) with exon | 10.8 |
| 307806 | AI351739 | Hs.276726 | EST singleton (not in UniGene) with exon | 4.7 |
| 308079 | AI472733 | Hs.270208 | ESTs | 4.2 |
| 308307 | AI581398 | Hs.172928 | collagen; type I; alpha 1 | 5.4 |
| 308511 | AI687580 | Hs.169476 | EST singleton (not in UniGene) with exon | 10.1 |
| 308615 | AI738593 | Hs.101774 | EST singleton (not in UniGene) with exon | 15.1 |
| 308677 | AI761173 | | EST singleton (not in UniGene) with exon | 4.6 |
| 308852 | AI829848 | Hs.182937 | peptidylprolyl isomerase A (cyclophilin A | 5.9 |
| 308974 | AI872290 | Hs.300697 | immunoglobulin gamma 3 (Gm marker) | 4.5 |
| 308981 | AI873242 | | EST singleton (not in UniGene) with exon | 7.6 |
| 308995 | AI880172 | | EST singleton (not in UniGene) with exon | 6.6 |
| 309177 | AI951118 | | EST singleton (not in UniGene) with exon | 24.3 |
| 309186 | AI952723 | Hs.90207 | EST singleton (not in UniGene) with exon | 6.1 |
| 309198 | AI955915 | | major histocompatibility complex; class I; | 5.6 |
| 309226 | AI969897 | | EST singleton (not in UniGene) with exon | 6.2 |
| 309279 | AI990102 | | EST singleton (not in UniGene) with exon | 7.9 |
| 309583 | AW170035 | | EST | 64.5 |
| 309624 | AW191929 | Hs.252989 | EST | 5.3 |
| 309629 | AW192764 | Hs.172928 | collagen; type I; alpha 1 | 6.9 |
| 309641 | AW194230 | Hs.253100 | EST | 11.4 |
| 309698 | AW238461 | Hs.73742 | ribosomal protein; large; P0 | 4.3 |
| 309700 | AW241170 | Hs.179661 | Homo sapiens clone 24703 beta-tubulin m | 11.9 |
| 310073 | AI335004 | Hs.148558 | ESTs | 4.2 |
| 310094 | AW450967 | Hs.235240 | ESTs | 5.7 |
| 310373 | AW080778 | Hs.145582 | ESTs | 4.8 |
| 310438 | AW022192 | Hs.200197 | ESTs | 39.1 |
| 310470 | AI281848 | Hs.194691 | ESTs | 4.9 |
| 310583 | AW205632 | Hs.211198 | ESTs | 7 |
| 310877 | T47784 | Hs.188955 | ESTs | 4.1 |
| 311067 | AI587332 | Hs.209115 | ESTs | 11.2 |
| 311166 | AI821294 | Hs.118599 | ESTs | 24.1 |
| 311199 | T57896 | Hs.191095 | EST cluster (not in UniGene) | 5.7 |
| 311465 | AI758660 | Hs.206132 | ESTs | 15.7 |
| 311587 | AI828254 | Hs.271019 | ESTs | 6.4 |
| 311774 | AA700870 | Hs.14304 | ESTs | 6.2 |
| 311785 | AI056769 | Hs.133512 | ESTs | 5 |
| 311923 | T60843 | Hs.189679 | ESTs | 5.9 |
| 311935 | AA216387 | | EST cluster (not in UniGene) | 5.5 |
| 311972 | N51511 | Hs.188449 | ESTs | 5.2 |
| 312014 | AI435650 | Hs.128778 | ESTs | 4.3 |
| 312047 | AA588275 | Hs.180669 | ESTs | 14.7 |
| 312147 | T89855 | Hs.195648 | EST cluster (not in UniGene) | 9.8 |
| 312153 | AA759250 | Hs.153028 | cytochrome b-561 | 27.1 |
| 312168 | T92251 | Hs.198882 | ESTs | 4.2 |
| 312172 | AI222168 | Hs.191168 | ESTs | 6.1 |
| 312226 | AI796815 | Hs.199993 | ESTs; Weakly similar to ubiquitous TPR | 5.5 |
| 312292 | AW451893 | Hs.151124 | ESTs | 18.4 |
| 312312 | AI080505 | Hs.134529 | ESTs | 11.9 |
| 312369 | AA582039 | Hs.173884 | Homo sapiens mRNA; chromosome 1 spe | 4 |
| 312407 | R46180 | Hs.153485 | ESTs | 13.6 |
| 312430 | AW139117 | Hs.117494 | ESTs | 4.1 |
| 312470 | AW451347 | Hs.175862 | ESTs | 4.6 |
| 312483 | AI417526 | Hs.7753 | ESTs | 15.3 |
| 312521 | AA033609 | Hs.319093 | ESTs | 12.5 |
| 312544 | AI498371 | Hs.183526 | ESTs | 14.6 |
| 312638 | AW439195 | Hs.256880 | ESTs | 5.3 |
| 312754 | R99834 | Hs.250383 | ESTs | 8.4 |
| 312772 | H63791 | | EST cluster (not in UniGene) | 4.3 |
| 312821 | AA699325 | Hs.269880 | ESTs | 8.3 |
| 312837 | AW292286 | Hs.255058 | ESTs | 7.1 |
| 312849 | AA846353 | Hs.194054 | ESTs | 5.9 |
| 312854 | AA828713 | Hs.321058 | EST cluster (not in UniGene) | 4.1 |
| 312992 | AA088446 | Hs.170298 | ESTs | 7.3 |
| 313096 | AI422367 | Hs.163533 | ESTs | 6.1 |
| 313112 | AA732534 | Hs.269099 | ESTs | 4.2 |
| 313126 | AA720887 | Hs.283313 | EST cluster (not in UniGene) | 18.1 |
| 313136 | N59284 | Hs.288010 | ESTs | 17 |
| 313197 | AI738851 | Hs.222487 | ESTs | 12.9 |
| 313219 | N74924 | Hs.182099 | ESTs | 7.1 |
| 313258 | AW068358 | Hs.183918 | ESTs | 13.7 |
| 313328 | AW449211 | Hs.105445 | ESTs | 27.9 |
| 313352 | AW292127 | Hs.144758 | ESTs | 9.8 |
| 313417 | AA741151 | Hs.137323 | ESTs | 8.2 |
| 313455 | AW081702 | Hs.98571 | ESTs | 6.9 |
| 313590 | AA804410 | Hs.291677 | EST cluster (not in UniGene) | 5.3 |
| 313663 | AI953261 | Hs.169813 | ESTs | 7.6 |
| 313667 | U69201 | Hs.13684 | ESTs; Weakly similar to choline kinase is | 12.5 |
| 313749 | AW450376 | Hs.119004 | ESTs | 5.5 |
| 313832 | AW271022 | Hs.133294 | ESTs | 4.3 |
| 313881 | AA535580 | Hs.16331 | ESTs | 7.7 |
| 313915 | AI969390 | Hs.163443 | ESTs | 27.1 |
| 313955 | AI858884 | Hs.270647 | ESTs | 5.7 |
| 313974 | AI310151 | Hs.173524 | ESTs | 4.3 |
| 314097 | AA648744 | Hs.269493 | ESTs | 14.5 |
| 314129 | AA228366 | Hs.115122 | ESTs | 9.5 |
| 314359 | AA205569 | Hs.194193 | ESTs | 5.4 |
| 314384 | AA535840 | Hs.162203 | ESTs; Weakly similar to alternatively spli | 5.3 |
| 314394 | AI380563 | Hs.130816 | ESTs | 13.2 |
| 314462 | AA347951 | Hs.326413 | ESTs | 6.2 |
| 314465 | AA602917 | Hs.156974 | ESTs | 18.1 |
| 314470 | AI934422 | Hs.30661 | ESTs | 4.2 |
| 314488 | AA358265 | Hs.182890 | ESTs | 6.1 |
| 314506 | AA833655 | Hs.206868 | ESTs | 27.8 |
| 314510 | AI204418 | Hs.190080 | ESTs | 9.5 |
| 314558 | AI873274 | Hs.190721 | ESTs | 22.5 |
| 314661 | AA436432 | Hs.324239 | EST cluster (not in UniGene) | 13.3 |
| 314691 | AW207206 | Hs.136319 | ESTs | 21.4 |
| 314754 | AW026761 | Hs.134374 | ESTs | 4.4 |
| 314775 | AI149880 | Hs.188809 | ESTs | 4.4 |
| 314943 | AI476797 | Hs.184572 | cell division cycle 2; G1 to S and G2 to M | 18.4 |
| 314961 | AW008061 | Hs.231994 | ESTs | 10.2 |
| 314963 | AI689617 | Hs.200934 | ESTs | 5.3 |
| 315006 | AI538613 | Hs.298241 | ESTs | 20.7 |
| 315010 | AA531082 | Hs.240049 | ESTs | 5 |
| 315019 | AA532807 | Hs.105822 | ESTs | 6.1 |
| 315033 | AI493046 | Hs.146133 | ESTs | 12 |
| 315036 | AA534953 | Hs.163297 | ESTs | 8.3 |
| 315037 | AW205863 | Hs.133988 | ESTs; Weakly similar to gene MAC25 pr | 6.1 |
| 315051 | AW292425 | Hs.163484 | EST | 12.7 |
| 315054 | AI968598 | Hs.78768 | ESTs | 7.6 |
| 315073 | AW452948 | Hs.257631 | ESTs | 13.9 |
| 315080 | AA744550 | Hs.136345 | ESTs | 4.4 |
| 315083 | AI221325 | Hs.205442 | ESTs | 5.1 |
| 315088 | AA557351 | Hs.152448 | ESTs; Moderately similar to MULTIFUN | 4.7 |
| 315175 | AI025842 | Hs.152530 | ESTs | 11.9 |
| 315196 | AA972756 | Hs.44898 | ESTs | 28.8 |
| 315296 | AA876905 | Hs.125286 | ESTs | 16.1 |
| 315303 | AW194364 | Hs.128022 | ESTs; Weakly similar to FIG-1 PROTEIN | 25.7 |
| 315352 | AA604799 | Hs.136528 | ESTs; Moderately similar to !!!! ALU SU | 12.3 |
| 315364 | AA643602 | Hs.155485 | ESTs; Highly similar to serine protease [H | 4.6 |
| 315368 | AW291563 | Hs.104696 | ESTs | 4.8 |
| 315390 | AI801565 | Hs.200113 | ESTs; Weakly similar to alternatively spli | 4.4 |
| 315408 | AW273261 | Hs.216292 | ESTs | 5 |
| 315458 | AA872000 | Hs.116104 | ESTs | 7.6 |
| 315472 | AA828850 | Hs.165469 | ESTs | 4.9 |
| 315478 | AA665612 | Hs.120874 | ESTs | 5.2 |
| 315498 | AA628539 | Hs.116252 | ESTs; Moderately similar to !!!! ALU SU | 4.8 |
| 315527 | AI791138 | Hs.116768 | ESTs | 4.4 |
| 315530 | AI200852 | Hs.127780 | ESTs | 22.4 |
| 315562 | AA737415 | Hs.152826 | ESTs | 5.9 |
| 315634 | AA837085 | Hs.220585 | ESTs | 8.8 |
| 315647 | AA648983 | Hs.212911 | ESTs | 15 |
| 315652 | AI521489 | Hs.3053 | ESTs | 6.3 |
| 315676 | AW002565 | Hs.124660 | ESTs | 9.2 |
| 315680 | AA814309 | Hs.123583 | ESTs | 8.1 |
| 315735 | AI831760 | Hs.155111 | ESTs | 13.4 |
| 315741 | AA812168 | Hs.122559 | ESTs | 5.4 |
| 315769 | AA744875 | Hs.189413 | ESTs | 4.4 |
| 315978 | AA830893 | Hs.119769 | ESTs | 10.4 |
| 315984 | AI015862 | Hs.131793 | ESTs | 5 |
| 316042 | AW297979 | Hs.170698 | ESTs | 14.7 |
| 316136 | AA830808 | Hs.124366 | ESTs | 4 |
| 316177 | AI908272 | Hs.293102 | EST cluster (not in UniGene) | 32.6 |
| 316313 | AA741300 | Hs.202599 | ESTs | 4.8 |
| 316405 | AA757900 | Hs.270823 | ESTs | 4.8 |
| 316480 | AI749921 | Hs.205377 | ESTs | 12.9 |
| 316564 | AI743571 | Hs.168799 | ESTs; Weakly similar to !!!! ALU SUBFA | 8.1 |
| 316714 | AA809792 | Hs.123307 | ESTs | 5 |
| 316715 | AI440266 | Hs.170673 | ESTs | 4.2 |
| 316828 | AA828116 | Hs.173076 | ESTs | 5.2 |
| 316869 | AI954880 | Hs.134604 | ESTs | 13.3 |
| 316905 | AW138241 | Hs.210846 | ESTs | 6.2 |
| 316943 | AW014875 | Hs.137007 | ESTs | 5.3 |
| 316949 | AA856749 | Hs.124620 | ESTs | 7.2 |
| 317008 | AW051597 | Hs.143707 | ESTs | 4.1 |
| 317028 | AA962623 | Hs.189144 | ESTs; Weakly similar to RENAL SODIU | 4.2 |
| 317067 | AI805392 | Hs.325335 | ESTs | 4.5 |
| 317069 | AI732892 | Hs.190489 | ESTs | 6.4 |
| 317210 | AA490718 | | EST cluster (not in UniGene) | 4.4 |
| 317298 | AI922374 | Hs.158549 | ESTs | 5.9 |
| 317658 | AW139077 | Hs.202217 | ESTs | 4.6 |
| 317674 | AW294909 | Hs.132208 | ESTs | 5.2 |
| 317685 | AI798630 | Hs.149997 | ESTs | 4.3 |
| 317836 | AA983913 | Hs.128929 | ESTs | 12.4 |
| 317881 | AI827248 | Hs.224398 | ESTs | 12.1 |
| 317902 | AI828602 | Hs.211265 | ESTs | 8.8 |
| 317916 | AI565071 | Hs.159983 | ESTs | 12.6 |
| 318042 | AW294522 | Hs.149991 | ESTs | 5.6 |
| 318053 | AI074465 | Hs.133469 | ESTs | 4 |
| 318064 | AW296888 | Hs.170939 | ESTs | 5.2 |
| 318070 | AI024594 | Hs.248942 | ESTs | 4.7 |
| 318073 | AW167087 | Hs.131562 | ESTs | 15.7 |
| 318146 | AI040125 | Hs.150521 | ESTs | 5.9 |
| 318186 | AW016773 | Hs.3709 | ESTs | 5.3 |
| 318481 | AI291584 | Hs.145921 | ESTs; Weakly similar to HYPOTHETICA | 7.6 |
| 318566 | AI335361 | Hs.226376 | ESTs | 5.8 |
| 318617 | AW247252 | Hs.75514 | nucleoside phosphorylase | 11.1 |
| 318662 | AI285898 | Hs.294014 | ESTs | 16.3 |
| 318691 | AW192139 | Hs.181307 | H3 histone; family 3A | 4 |
| 318740 | NM_002543 | Hs.77729 | EST cluster (not in UniGene) | 21.3 |
| 318744 | AI793124 | Hs.144479 | ESTs | 35 |
| 318948 | AA317274 | Hs.13996 | ESTs | 11.7 |
| 319163 | F15257 | Hs.27 | glycine dehydrogenase (decarboxylating; | 7 |
| 319478 | R06841 | Hs.270307 | EST cluster (not in UniGene) | 8.9 |
| 319545 | R83716 | Hs.14355 | ESTs | 8.2 |
| 319668 | NM_002731 | Hs.87773 | EST cluster (not in UniGene) | 25.4 |
| 319763 | AA460775 | Hs.6295 | ESTs | 7 |
| 319913 | AA179304 | Hs.271586 | ESTs; Moderately similar to !!!! ALU SU | 8.7 |
| 319936 | W22152 | Hs.282929 | EST cluster (not in UniGene) | 5.6 |
| 319951 | AA307665 | Hs.14559 | ESTs | 4.9 |
| 319962 | H06350 | Hs.135056 | ESTs | 9.2 |
| 319977 | AA632632 | | EST cluster (not in UniGene) | 4.6 |
| 320074 | AA321166 | Hs.278233 | EST cluster (not in UniGene) | 16.7 |
| 320092 | AF022799 | Hs.113292 | calpain 9 (nCL-4) | 5.4 |
| 320107 | AA836461 | Hs.291712 | EST cluster (not in UniGene) | 5.3 |
| 320133 | D63271 | | EST cluster (not in UniGene) | 5.5 |
| 320167 | AA984373 | Hs.90790 | EST cluster (not in UniGene) | 15 |
| 320187 | T99949 | Hs.303428 | EST cluster (not in UniGene) | 6.7 |
| 320211 | AL039402 | Hs.125783 | DEME-6 protein | 24.3 |
| 320401 | U90449 | Hs.152717 | nucleoside diphosphate kinase type 6 (inh | 10 |
| 320458 | AI884396 | Hs.24131 | ESTs | 5.4 |
| 320488 | R31386 | Hs.191791 | EST cluster (not in UniGene) | 4.9 |
| 320521 | N31464 | Hs.24743 | ESTs | 9.5 |
| 320661 | AA864846 | Hs.115175 | EST cluster (not in UniGene) | 6.6 |
| 320691 | R61576 | Hs.313951 | hypothetical protein | 5.9 |
| 320699 | R63161 | Hs.118249 | EST cluster (not in UniGene) | 4 |
| 320727 | U96044 | Hs.181125 | EST cluster (not in UniGene) | 15.3 |
| 320993 | AL050145 | Hs.225986 | Homo sapiens mRNA; cDNA DKFZp586 | 7.2 |
| 321012 | AA737314 | Hs.194324 | EST cluster (not in UniGene) | 6.1 |
| 321050 | AW393497 | | EST cluster (not in UniGene) | 5 |
| 321051 | AF134149 | Hs.240395 | EST cluster (not in UniGene) | 11.4 |
| 321171 | AI769410 | Hs.221461 | ESTs | 7.7 |
| 321192 | AA295304 | Hs.297939 | ESTs; Weakly similar to neogenin [H. sap | 5.5 |
| 321354 | AA078493 | | EST cluster (not in UniGene) | 16.9 |
| 321387 | H68014 | Hs.141278 | ESTs; Weakly similar to !!!! ALU SUBFA | 4.2 |
| 321412 | AW366305 | Hs.22891 | EST cluster (not in UniGene) | 6.3 |
| 321489 | AW392474 | Hs.172759 | ESTs; Moderately similar to !!!! ALU SU | 9 |
| 321539 | N98619 | Hs.42915 | ARP2 (actin-related protein 2; yeast) hom | 11.3 |
| 321593 | H84762 | Hs.253197 | ESTs | 10.4 |
| 321666 | D28390 | Hs.272897 | EST cluster (not in UniGene) | 19.9 |
| 321891 | AW157424 | Hs.165954 | ESTs | 5.6 |
| 321910 | H67065 | Hs.271530 | ESTs; Weakly similar to !!!! ALU SUBFA | 5.4 |
| 321953 | AW068268 | Hs.292833 | ESTs; Weakly similar to !!!! ALU CLASS | 6.5 |
| 321978 | N77342 | Hs.21851 | EST cluster (not in UniGene) | 10.2 |
| 322017 | AA310039 | Hs.9192 | ESTs | 9.8 |
| 322026 | AA233527 | Hs.283675 | low density lipoprotein receptor (familial | 27.8 |
| 322035 | AL137517 | Hs.306201 | EST cluster (not in UniGene) | 40.2 |
| 322171 | AF085968 | Hs.48474 | EST cluster (not in UniGene) | 5.7 |
| 322175 | AF085975 | | EST cluster (not in UniGene) | 7.7 |
| 322236 | AL134970 | Hs.104222 | follistatin-like 1 | 14.4 |
| 322303 | W07459 | Hs.157601 | EST cluster (not in UniGene) | 13.4 |
| 322735 | AA086123 | Hs.297856 | EST cluster (not in UniGene) | 7.6 |
| 322777 | AA679082 | Hs.269947 | ESTs | 4.4 |
| 322818 | AW043782 | Hs.293616 | ESTs | 21 |
| 322882 | AW248508 | Hs.279727 | DiGeorge syndrome critical region gene 2 | 15.3 |
| 322975 | C16391 | | EST cluster (not in UniGene) | 21.3 |
| 322991 | C18965 | Hs.159473 | ESTs | 11.7 |
| 323011 | AA580288 | | EST cluster (not in UniGene) | 8.9 |
| 323091 | AW014094 | Hs.210761 | ESTs | 10.8 |
| 323107 | AI301107 | Hs.150790 | ESTs | 6.5 |
| 323136 | AL120351 | Hs.30177 | EST cluster (not in UniGene) | 5.5 |
| 323168 | AL120862 | Hs.124165 | ESTs | 17.9 |
| 323195 | AI064982 | Hs.117950 | multifunctional polypeptide similar to SA | 5.8 |
| 323201 | AL049370 | Hs.13350 | Homo sapiens mRNA; cDNA DKFZp586 | 11.6 |
| 323203 | AA203135 | Hs.130186 | ESTs | 6.4 |
| 323243 | W44372 | Hs.110771 | EST cluster (not in UniGene) | 7.3 |
| 323244 | T70731 | Hs.193620 | EST cluster (not in UniGene) | 15.8 |
| 323328 | AA228078 | Hs.255096 | EST cluster (not in UniGene) | 4.8 |
| 323332 | AI829520 | Hs.227513 | ESTs | 20.2 |
| 323333 | AA228883 | Hs.208558 | EST cluster (not in UniGene) | 8.8 |
| 323570 | AL038623 | Hs.208752 | ESTs; Weakly similar to !!!! ALU SUBFA | 5 |
| 323604 | AI751438 | Hs.41271 | ESTs; Weakly similar to !!!! ALU SUBFA | 6.5 |
| 323685 | AA344205 | Hs.289088 | EST cluster (not in UniGene) | 7.1 |
| 323753 | AA327102 | Hs.70266 | EST cluster (not in UniGene) | 6.1 |
| 323817 | AA410943 | | EST cluster (not in UniGene) | 16.8 |
| 323845 | AI684674 | Hs.41127 | ESTs; Weakly similar to waclaw [D. melan | 10.1 |
| 323930 | AA570698 | Hs.8173 | ESTs | 6.4 |
| 323997 | AA844907 | Hs.274454 | EST cluster (not in UniGene) | 8 |
| 324047 | AA378201 | Hs.271340 | EST cluster (not in UniGene) | 6.3 |
| 324261 | AL044891 | Hs.269350 | EST cluster (not in UniGene) | 50.1 |
| 324302 | AA543008 | Hs.292471 | ESTs; Weakly similar to !!!! ALU SUBFA | 5.7 |
| 324338 | AL138357 | Hs.145078 | ESTs | 9.5 |
| 324344 | AW502000 | Hs.46677 | EST cluster (not in UniGene) | 4.4 |
| 324432 | AA464510 | Hs.152812 | EST cluster (not in UniGene) | 16.7 |
| 324495 | AW501411 | Hs.122489 | ESTs; Weakly similar to !!!! ALU CLASS | 5.5 |
| 324497 | AW152624 | Hs.136340 | ESTs | 5.4 |
| 324598 | AA502659 | Hs.163986 | ESTs | 8.8 |
| 324603 | AW016378 | Hs.292934 | ESTs | 23.1 |
| 324620 | AA448021 | Hs.94109 | EST cluster (not in UniGene) | 21.2 |
| 324727 | AI610425 | Hs.19597 | ESTs | 5 |
| 324774 | AI031771 | Hs.132586 | ESTs | 5 |
| 324783 | AA640770 | Hs.200994 | EST cluster (not in UniGene) | 4.1 |
| 324824 | AI826999 | Hs.224624 | ESTs | 6.3 |
| 324826 | AA704806 | Hs.143842 | ESTs | 11.7 |
| 324902 | D31323 | Hs.271492 | ESTs | 4.8 |
| 324961 | AA613792 | | EST cluster (not in UniGene) | 13.3 |
| 324987 | T06882 | Hs.172634 | ESTs | 19.6 |
| 324988 | T06997 | Hs.121028 | EST cluster (not in UniGene) | 24.5 |
| 325146 | AI064690 | Hs.171176 | ESTs | 4.6 |
| 325622 | | | CH.14_hs gi\|5867000 | 5.2 |
| 326213 | | | CH.17_hs gi\|5867224 | 8.1 |
| 326474 | | | CH.19_hs gi\|5867405 | 12.7 |
| 326816 | | | CH.20_hs gi\|6552458 | 9.4 |
| 326817 | | | CH.20_hs gi\|6552458 | 11.7 |
| 327110 | | | CH.21_hs gi\|6117842 | 14.7 |
| 327196 | | | CH.01_hs gi\|5867446 | 5.1 |
| 327283 | | | CH.01_hs gi\|5867478 | 4.3 |
| 327313 | | | CH.01_hs gi\|5867501 | 4.8 |
| 327450 | | | CH.02_hs gi\|5867766 | 4.1 |
| 328059 | | | CH.06_hs gi\|6117819 | 6.2 |
| 328304 | | | CH.07_hs gi\|6004478 | 5.4 |
| 328492 | | | CH.07_hs gi\|5868455 | 7 |
| 328857 | | | CH.07_hs gi\|6381927 | 5.2 |
| 329367 | | | CH.X_hs gi\|5868842 | 7.6 |
| 329373 | | | CH.X_hs gi\|6682537 | 12 |
| 329655 | | | CH.14_p2 gi\|6448516 | 4 |
| 329899 | | | CH.15_p2 gi\|6563505 | 4 |
| 329960 | | | CH.16_p2 gi\|5091594 | 7.6 |
| 330084 | | | CH.19_p2 gi\|6015302 | 4 |
| 330384 | M23263 | | androgen receptor (dihydrotestosterone re | 5.8 |
| 330385 | AA449749 | | ESTs; Highly similar to secreted apoptosi | 10.2 |
| 330387 | H14624 | | ESTs; Highly similar to secreted apoptosi | 4.4 |
| 330388 | X03363 | | HER2 receptor tyrosine kinase (c-erbB-2; | 17.7 |
| 330409 | D50692 | Hs.78221 | c-myc binding protein | 10.1 |
| 330460 | TIGR: HT544 | Hs.73946 | Endothelial Cell Growth Factor 1 | 5.5 |
| 330486 | M13755 | Hs.833 | interferon-stimulated protein; 15 kDa | 67 |
| 330494 | M29696 | Hs.237868 | interleukin 7 receptor | 6 |
| 330500 | M34423 | Hs.79222 | galactosidase; beta 1 | 13.1 |
| 330510 | M75099 | Hs.227729 | FK506-binding protein 2 (13 kD) | 29 |
| 330513 | M81057 | Hs.180884 | carboxypeptidase B1 (tissue) | 38.5 |
| 330541 | U22970 | Hs.265827 | multiple UniGene matches | 7.4 |
| 330542 | U23942 | Hs.226213 | cytochrome P450; 51 (lanosterol 14-alpha | 15 |
| 330547 | U32989 | Hs.183671 | tryptophan 2; 3-dioxygenase | 11 |
| 330551 | U39840 | Hs.299867 | hepatocyte nuclear factor 3; alpha | 6.5 |
| 330562 | U49082 | Hs.76460 | transporter protein | 7.7 |
| 330573 | U62800 | Hs.83393 | cystatin E/M | 4 |
| 330673 | D57823 | Hs.321403 | Sec23 (S. cerevisiae) homolog A | 10.5 |
| 330711 | AA164687 | Hs.177576 | mannosyl (alpha-1; 3-)-glycoprotein beta-1 | 24.3 |
| 330814 | AA015730 | Hs.265398 | ESTs; Weakly similar to transformation-r | 44.1 |
| 330850 | AA075298 | Hs.322710 | ESTs | 4.4 |
| 330874 | AA127474 | Hs.191157 | ESTs; Weakly similar to !!!! ALU SUBFA | 8.1 |
| 330884 | AA133457 | Hs.102548 | ESTs | 5.2 |
| 330912 | AA195936 | Hs.82719 | general transcription factor IIA; 1 (37 kDa | 5 |
| 330924 | AA232136 | Hs.159737 | Homo sapiens mRNA; cDNA DKFZp434 | 9.1 |
| 330997 | H55762 | Hs.9302 | ESTs | 7.6 |
| 331014 | H98597 | Hs.30340 | ESTs | 13.5 |
| 331024 | N32919 | Hs.27931 | ESTs | 9.1 |
| 331046 | N66563 | Hs.191358 | ESTs | 10.5 |
| 331135 | R61398 | Hs.4197 | ESTs | 7.4 |
| 331145 | R72427 | Hs.129873 | ESTs; Weakly similar to CYTOCHROME | 41.9 |
| 331148 | R73816 | Hs.17385 | ESTs | 4.7 |
| 331222 | T98531 | Hs.173904 | ESTs | 4.1 |
| 331230 | W69807 | Hs.16537 | hypothetical protein; similar to (U06944) | 4.9 |
| 331306 | AA252079 | Hs.63931 | dachshund (Drosophila) homolog | 15.1 |
| 331327 | AA281076 | Hs.109221 | ESTs | 4.8 |
| 331337 | AA287662 | Hs.50495 | ESTs | 7.6 |
| 331341 | AA303125 | Hs.23240 | ESTs; Weakly similar to !!!! ALU SUBFA | 13 |
| 331344 | AA357927 | Hs.126550 | ESTs | 12.4 |
| 331362 | AA417956 | Hs.40782 | ESTs | 6.5 |
| 331363 | AA421562 | Hs.91011 | anterior gradient 2 (Xenepus laevis) homo | 28.2 |
| 331376 | AA443802 | Hs.41007 | ESTs; Weakly similar to cDNA EST yk47 | 15.1 |
| 331384 | AA456001 | Hs.93847 | ESTs | 7.9 |
| 331478 | N26608 | Hs.40639 | ESTs | 7 |
| 331526 | N49967 | Hs.46624 | ESTs | 19.8 |
| 331533 | N51517 | Hs.47282 | ESTs | 6.5 |
| 331681 | W85712 | Hs.119571 | collagen; type III; alpha 1 (Ehlers-Danlos | 13.8 |
| 331686 | W88502 | Hs.182258 | ESTs | 9.9 |
| 331750 | AA284372 | Hs.111471 | ESTs | 5.6 |
| 331751 | AA284840 | Hs.143818 | ESTs | 5.8 |
| 331760 | AA292721 | Hs.154434 | ESTs; Weakly similar to unknown [H. sap | 7.4 |
| 331763 | AA312861 | Hs.96704 | ESTs | 7.8 |
| 331825 | AA411144 | Hs.292882 | ESTs | 15.2 |
| 331890 | AA432166 | Hs.3577 | succinate dehydrogenase complex; subuni | 24.3 |
| 331952 | AA454756 | Hs.97837 | ESTs | 5 |
| 332015 | AA487910 | Hs.208800 | ESTs; Weakly similar to !!!! ALU CLASS | 10.5 |
| 332043 | AA490831 | Hs.125056 | ESTs | 11.4 |
| 332060 | AA504779 | Hs.191402 | ESTs | 13.6 |
| 332071 | AA598594 | Hs.205293 | ESTs | 9.1 |
| 332093 | AA608794 | Hs.112592 | ESTs | 8.8 |
| 332139 | AA620669 | Hs.112879 | EST | 9 |
| 332219 | N22508 | Hs.139315 | ESTs | 7.1 |
| 332225 | N33213 | Hs.100425 | ESTs | 12.2 |
| 332246 | N57927 | Hs.120777 | ESTs; Weakly similar to RNA POLYME | 15.6 |
| 332247 | N58172 | Hs.109370 | ESTs | 16.9 |
| 332260 | N70088 | Hs.138467 | ESTs | 4 |
| 332269 | N91279 | Hs.109654 | ESTs; Moderately similar to outer membr | 8.2 |
| 332336 | T96130 | Hs.137551 | ESTs | 7.7 |
| 332340 | W15495 | Hs.129781 | chromosome 21 open reading frame 5 | 14.1 |
| 332347 | W60326 | Hs.288684 | ESTs | 4.4 |
| 332362 | W93640 | Hs.4779 | ESTs; Moderately similar to similar to AD | 16.9 |
| 332467 | AA489630 | Hs.119004 | KIAA0665 gene product | 4.8 |
| 332499 | M12036 | Hs.323910 | Human tyrosine kinase-type receptor (HE | 10.4 |
| 332513 | AA018182 | Hs.154424 | deiodinase; iodothyronine; type II | 5.8 |
| 332526 | AA281753 | Hs.77515 | inositol 1; 4; 5-triphosphate receptor; type | 19 |
| 332532 | N63192 | Hs.1892 | EST; Highly similar to PHENYLETHAN | 15.3 |
| 332565 | AA234896 | Hs.25272 | E1A binding protein p300 | 12.3 |
| 332607 | R41791 | Hs.36566 | LIM domain kinase 1 | 11.1 |
| 332640 | AA417152 | Hs.5101 | protein regulator of cytokinesis 1 | 18.2 |
| 332694 | AA262768 | Hs.243901 | KIAA1067 protein | 15.2 |
| 332702 | H93968 | Hs.75725 | transgelin 2 | 4.7 |
| 332705 | T59161 | Hs.76293 | thymosin; beta 10 | 5.5 |
| 332749 | AA479968 | Hs.88251 | arylsulfatase A | 9.8 |
| 332927 | | | CH22_FGENES.38_1 | 17.7 |
| 332929 | | | CH22_FGENES.38_3 | 4.7 |
| 332930 | | | CH22_FGENES.38_4 | 7.4 |
| 332955 | | | CH22_FGENES.48_12 | 5.4 |
| 332958 | | | CH22_FGENES.48_15 | 17.8 |
| 332961 | | | CH22_FGENES.48_18 | 10.6 |
| 332983 | | | CH22_FGENES.54_5 | 4.3 |
| 333009 | | | CH22_FGENES.61_1 | 5.2 |
| 333010 | | | CH22_FGENES.61_2 | 8.1 |
| 333013 | | | CH22_FGENES.61_5 | 8.5 |
| 333108 | | | CH22_FGENES.79_14 | 5.6 |
| 333139 | | | CH22_FGENES.83_16 | 6.3 |
| 333254 | | | CH22_FGENES.118_2 | 6.8 |
| 333305 | | | CH22_FGENES.137_2 | 11.4 |
| 333343 | | | CH22_FGENES.139_12 | 5.1 |
| 333388 | | | CH22_FGENES.144_3 | 12.7 |
| 333456 | | | CH22_FGENES.157_5 | 4.2 |
| 333459 | | | CH22_FGENES.157_8 | 7.6 |
| 333517 | | | CH22_FGENES.173_2 | 8.2 |
| 333585 | | | CH22_FGENES.203_4 | 5 |
| 333679 | | | CH22_FGENES.247_6 | 4.3 |
| 333743 | | | CH22_FGENES.264_1 | 13.4 |
| 333758 | | | CH22_FGENES.268_1 | 4 |
| 333767 | | | CH22_FGENES.271_6 | 5.6 |
| 333768 | | | CH22_FGENES.271_7 | 12.2 |
| 333769 | | | CH22_FGENES.271_8 | 48.3 |
| 333795 | | | CH22_FGENES.275_1 | 6.1 |
| 333796 | | | CH22_FGENES.275_3 | 6.8 |
| 333892 | | | CH22_FGENES.292_14 | 4.4 |
| 333904 | | | CH22_FGENES.294_2 | 6.5 |
| 333905 | | | CH22_FGENES.294_3 | 9.3 |
| 333921 | | | CH22_FGENES.296_12 | 9.6 |
| 333968 | | | CH22_FGENES.307_4 | 15.9 |
| 334102 | | | CH22_FGENES.327_60 | 7.1 |
| 334222 | | | CH22_FGENES.360_3 | 6.7 |
| 334223 | | | CH22_FGENES.360_4 | 33.5 |
| 334264 | | | CH22_FGENES.367_15 | 18.5 |
| 334343 | | | CH22_FGENES.375_25 | 6.1 |
| 334360 | | | CH22_FGENES.378_5 | 6.1 |
| 334784 | | | CH22_FGENES.432_9 | 4.8 |
| 334789 | | | CH22_FGENES.432_14 | 5.1 |
| 334794 | | | CH22_FGENES.434_2 | 7 |
| 334889 | | | CH22_FGENES.452_3 | 12.4 |
| 335004 | | | CH22_FGENES.472_8 | 7.9 |
| 335115 | | | CH22_FGENES.496_2 | 18.8 |
| 335287 | | | CH22_FGENES.526_11 | 4.5 |
| 335342 | | | CH22_FGENES.536_1 | 5.3 |
| 335491 | | | CH22_FGENES.570_23 | 24 |
| 335495 | | | CH22_FGENES.570_28 | 7 |
| 335498 | | | CH22_FGENES.571_7 | 12.2 |
| 335544 | | | CH22_FGENES.576_5 | 8.4 |
| 335610 | | | CH22_FGENES.583_4 | 12.9 |
| 335653 | | | CH22_FGENES.590_4 | 6.7 |
| 335682 | | | CH22_FGENES.595_2 | 12.1 |
| 335687 | | | CH22_FGENES.596_2 | 13.9 |
| 335755 | | | CH22_FGENES.604_4 | 11.5 |
| 335782 | | | CH22_FGENES.609_4 | 17.9 |
| 335791 | | | CH22_FGENES.611_7 | 27.3 |
| 335809 | | | CH22_FGENES.617_6 | 19.2 |
| 335822 | | | CH22_FGENES.619_7 | 19.1 |
| 335823 | | | CH22_FGENES.619_8 | 4.5 |
| 335824 | | | CH22_FGENES.619_11 | 40.2 |
| 335825 | | | CH22_FGENES.619_12 | 34.3 |
| 335895 | | | CH22_FGENES.635_3 | 10.2 |
| 335917 | | | CH22_FGENES.636_13 | 6 |
| 335920 | | | CH22_FGENES.636_16 | 8.8 |
| 336035 | | | CH22_FGENES.678_6 | 5.9 |
| 336042 | | | CH22_FGENES.679_4 | 5.8 |
| 336093 | | | CH22_FGENES.691_2 | 11.6 |
| 336096 | | | CH22_FGENES.691_5 | 7.6 |
| 336150 | | | CH22_FGENES.706_6 | 6.3 |
| 336152 | | | CH22_FGENES.706_9 | 10.5 |
| 336416 | | | CH22_FGENES.823_38 | 5 |
| 336444 | | | CH22_FGENES.827_10 | 4.8 |
| 336449 | | | CH22_FGENES.829_6 | 13.6 |
| 336471 | | | CH22_FGENES.829_30 | 6.9 |
| 336512 | | | CH22_FGENES.834_7 | 21.4 |
| 336558 | | | CH22_FGENES.842_3 | 8.2 |
| 336560 | | | CH22_FGENES.842_5 | 9 |
| 336676 | | | CH22_FGENES.43-4 | 9.4 |
| 336959 | | | CH22_FGENES.367-13 | 19 |
| 337968 | | | CH22_EM: AC005500.GENSCAN.103-2 | 13.4 |
| 338008 | | | CH22_EM: AC005500.GENSCAN.127-9 | 15.2 |
| 338057 | | | CH22_EM: AC005500.GENSCAN.160-1 | 13.9 |
| 338410 | | | CH22_EM: AC005500.GENSCAN.341-6 | 8 |
| 338451 | | | CH22_EM: AC005500.GENSCAN.359-3 | 11.6 |
| 338588 | | | CH22_EM: AC005500.GENSCAN.432-1 | 10.3 |
| 338665 | | | CH22_EM: AC005500.GENSCAN.464-2 | 4.8 |
| 338689 | | | CH22_EM: AC005500.GENSCAN.475-3 | 6.7 |
| 338832 | | | CH22_DJ246D7.GENSCAN.6-9 | 4.8 |
| 338980 | | | CH22_DA59H18.GENSCAN.2-4 | 5.1 |
| 339352 | | | CH22_BA354I12.GENSCAN.29-7 | 6.9 |
| 339373 | | | CH22_BA232E17.GENSCAN.1-29 | 4.3 |

Pkey: Unique Eos probeset identifier number
ExAccn: Exemplar Accession number, Genbank accession number
UnigeneID: Unigene number
Unigene Title: Unigene gene title
R1: Ratio of tumor to normal body tissue

TABLE 13A
Table 13A shows the accession numbers for those pkeys lacking unigeneID's for Table 13.
For each probeset, we have listed the gene cluster number from which the oligonucleotides
were designed. Gene clusters were compiled using sequences derived from Genbank ESTs
and mRNAs. These sequences were clustered based on sequence similarity using Clustering
and Alignment Tools (DoubleTwist, Oakland California). The Genbank accession numbers
for sequences comprising each cluster are listed in the “Accession” column.

| Pkey | CAT number | Accession |
|---|---|---|
| 123619 | 371681_1 | AA602964 AA609200 |
| 103207 | 30635_−4 | X72790 |
| 103349 | 11052_−2 | X89059 |
| 110856 | 19346_14 | AA992380 N33063 N21418 H79958 R21911 H79957 |
| 113248 | 328626_1 | T63857 AW971220 AA493469 T63699 |
| 123169 | 44573_2 | AI950087 N70208 R97040 N36809 AI308119 AW967677 N35320 AI251473 H59397 AW971573 |
| | | R97278 W01059 AW967671 AA908598 AA251875 AI820501 AI820532 W87891 T85904 U71456 |
| | | T82391 BE328571 T75102 R34725 AA884922 BE328517 AI219788 AA884444 N92578 F13493 |
| | | AA927794 AI560251 AW874068 AL134043 AW235363 AA663345 AW008282 AA488964 |
| | | AA283144 AI890387 AI950344 AI741346 AI689062 AA282915 AW102898 AI872193 AI763273 |
| | | AW173586 AW150329 AI653832 AI762688 AA988777 AA488892 |
| | | AI356394 AW103813 AI539642 AA642789 AA856975 AW505512 AI961530 AW629970 |
| | | BE612881 AW276997 AW513601 AW512843 AA044209 AW856538 AA180009 AA337499 |
| | | AW961101 AA251669 AA251874 AI819225 AW205862 AI683338 AI858509 AW276905 |
| | | AI633006 AA972584 AA908741 AW072629 AW513996 AA293273 AA969759 N75628 N22388 |
| | | H84729 H60052 T92487 AI022058 AA780419 AA551005 W80701 AW613456 AI373032 |
| | | AI564269 F00531 H83488 W37181 W78802 R66056 AI002839 R67840 AA300207 |
| | | AW959581 T63226 F04005 |
| 123533 | genbank_AA608751 | AA608751 |
| 116480 | genbank_C14088 | C14088 |
| 132225 | genbank_AA128980 | AA128980 |
| 125154 | genbank_W38419 | W38419 |
| 118475 | genbank_N66845 | N66845 |
| 102919 | 25180_2 | M21191 AL035748 AA021266 AA323126 AA180515 AI613029 D28356 NM_000034 M11560 |
| | | AW401425 AW248248 AA012956 AA323294 W04965 H38759 AA206622 AA580747 |
| | | AI541172 AA381075 AA354229 AW402353 AW405575 AW404021 AW406207 |
| | | AA075752 AW176066 AA287222 AA195818 T20243 R87945 AA295539 AA402533 |
| | | AA232419 AA224515 AW401583 AA331367 AW402140 AW249079 |
| | | R31488 AA075757 X05236 AW239490 AA338036 AW239495 AA357262 AA431005 |
| | | AA306726 R33804 AA216544 AW275288 AA227044 AL038124 AA243300 C03242 |
| | | AA315615 AL035840 R64336 AA313917 AA018963 AA001385 AA054395 H30840 AW498825 |
| | | AA086141 AI557324 AA121576 H39128 R77161 AA019688 AA380987 AA348140 AA348257 |
| | | AW176086 AA362432 AA171389 AA362416 AA299938 AA319093 AA337972 C04921 AA345696 |
| | | R89640 AA085425 AA481708 AA313637 AL039229 H84490 |
| | | H86153 F00656 AA326668 AA347304 R65890 H41949 AA339309 AW402002 AW404854 |
| | | AA192582 AA112802 H09248 N83165 H38367 AA356339 AA455763 R66853 AA294935 H85911 |
| | | AA310414 H93436 N87014 AA001186 H83640 AA411328 AA317929 |
| | | C04192 AW406288 U46335 AA323179 AA427649 AI366131 H14328 AA197161 |
| | | AA379497 AA311816 AA017206 AA001137 AA017420 AA012990 AW163775 AA021397 |
| | | AA295513 AA355248 AA374921 AW380419 AA345864 AA318058 AA371711 AA363255 |
| | | AA057094 R88057 AA394045 AW362741 AA479579 AW362789 AW362775 AA223624 |
| | | AW362809 AW362746 AW362753 AW380412 AA057104 AA295271 X06352 AW362771 |
| | | X12447 R59553 R87925 AA292864 R83920 F01120 AA496740 AA852551 AL039209 |
| | | AA093339 AA333961 N86416 C03325 C03831 AA194750 |
| | | AA359270 AA359460 AW245492 AA339400 AA330784 AI908970 N89223 AW361938 |
| | | AI940063 AA134323 AA371741 F01267 AW372970 AA341973 AA346098 AW372969 |
| | | AA337549 AA327342 H93855 AI074079 F29118 AA852940 F35696 AA345963 |
| | | AA079578 AA113785 C02989 AA095945 C03145 C05199 AA346024 AA190506 AW361659 |
| | | AI909845 AW374374 AW374382 AW374401 AW374373 AW374370 |
| | | AI909831 AW374367 AA353658 F01041 C02843 AA375948 AW374414 AA213946 AI525039 |
| | | H13744 R31007 AA112044 AA134404 H47935 AW177018 AA429768 AA336873 AA112875 |
| | | H46393 AA191267 D59131 AW406037 AA055244 AA341880 AA179024 AA308537 |
| | | AW406985 AW327311 H30301 AA300705 H43788 AW364149 AA806213 AA481936 C04941 |
| | | AW375299 M21190 AA410818 AA250940 AI354547 AA317422 AA250903 AI865497 AA890603 |
| | | AA366197 AW498538 M78072 AW406461 C03092 F00308 H56488 AA336320 AW406501 |
| | | AA354102 AA382942 AA096393 AW376830 AA383446 F01259 |
| | | AW081388 T94036 AA379643 H43842 AI524063 AA621727 AA379099 AA371417 R66811 |
| | | H44129 N84794 F01135 AA477852 AA293062 AW361595 H27194 AI831650 H43253 H24797 |
| | | AI564680 AA380090 W20057 AI921586 AI192549 AW090808 H25967 |
| | | AA918121 AI626060 H20221 AA812572 H42178 AA887222 H96000 C03180 F00946 |
| | | C03986 AI318091 AI860172 AA582179 AI633388 AA557193 R68075 F24105 AW518239 |
| | | W56622 AI625219 AI925243 AW468046 AI921828 AA339164 AI144391 AA643334 |
| | | AA459631 AA873247 AW373432 AA604384 H27600 AI680458 AA159956 AA610836 AA364298 |
| | | AW373435 AA604225 W73754 AW087924 AA599776 N89227 AI630871 AI633128 |
| | | AW514329 AA010455 AA563928 AI571596 AI128394 W73707 AI423575 AA563809 |
| | | AA657988 AI950837 AA169782 AA600009 AI885540 |
| | | AA771884 AI978829 AA505408 AA533937 AA481469 AA610869 AA775241 AW273870 |
| | | AW070909 AI905695 AA480115 AA574051 AI889185 AA773167 AA331375 AA001437 |
| | | AA194324 AA194300 AA558632 AI038538 AA411329 AA781570 AI833176 |
| | | AA935520 AW074197 AA583063 AW073099 AW001198 AA948025 AA587857 AA191540 |
| | | AI460085 AA193244 AI538037 AA515572 AA758587 AI149311 AA508610 |
| | | AA206409 AA534004 AA994600 AA827543 AI916349 AW245129 AW517804 D25663 |
| | | AA781985 AA284536 AI819422 H16040 H27531 AA456564 AA845555 AI423596 AA012908 |
| | | AA889439 AA716311 AA968868 AA320508 AA725731 AA834202 AA935997 AA724815 |
| | | AA769353 AA594803 F00827 AI342442 AI003519 AI002503 AI347597 AI040946 AA197162 |
| | | AA987883 AA292865 AW001944 AI640711 AW244044 AA456784 F30588 AA290829 |
| | | H24754 AI978683 AA483686 AA583939 AA121382 AA833831 AA477102 AA977322 |
| | | AA666379 F35456 AA993537 AI749610 AA226934 AA716204 AW513025 AA628543 |
| | | AA583705 F25702 AI368748 AI124097 AI880086 AA477513 AI758834 AI690753 AA477746 |
| | | F37761 AA642243 AA159957 AA250844 AA459406 AA427566 F25054 AI569314 AA961665 |
| | | AI922050 AI759000 AA555236 AA514432 AA293474 AA001129 AA826789 AA641390 |
| | | AA134405 F35585 AA477416 AW193359 AI361315 AA284988 F36340 AI361322 |
| | | F26969 AA991922 AA021267 F26973 AI361314 F35891 AI918509 |
| | | AA250964 AA190992 AA577139 AA865535 AA134324 AW192842 AI224046 F18975 |
| | | AA779626 AA856894 AW269997 AW014614 H95554 F31378 AA374868 F26343 AA654007 |
| | | AI830942 AA113195 F26432 W56652 AA464690 AA055263 AA340654 AA031448 AA976399 |
| | | AA972526 AA063476 R83921 T16240 AA533290 N91545 H44053 AA883451 AA513761 |
| | | AA086477 H09249 F20482 F26737 AA054148 AA857063 AA017259 AA179789 AA088908 |
| | | H43704 AA194320 F35950 AI880127 F20441 F32878 AA962483 H39094 H56489 H44621 F19390 |
| | | AI302232 F25162 AA826965 AA086052 AA917410 AA454513 R59554 AA196755 AA086369 |
| | | AA079530 H28106 AA243301 AI025737 AA101239 AA088887 AI214910 AA974886 F16089 |
| | | F26054 AA515092 F33436 F32829 M78061 AA235645 F19715 F37529 AI811549 AA665180 |
| | | AA708200 F01124 F32382 AA346220 AA627361 F30741 F30010 F28543 AA211715 F20245 |
| | | AA331222 F25634 F21996 W28215 F21911 R65793 AI192566 H20130 H84491 AA719223 |
| | | AA557435 F16967 F26989 F30353 AA857159 AA291918 F28234 F20840 F25176 F22437 |
| | | T27904 AA480355 F19528 R87926 H14286 F27532 AW337864 F28411 H13692 |
| | | F25651 AA975454 F24229 F29657 F18024 AA464779 F17588 F34954 |
| | | AA947328 F18063 AA657777 AA459644 N91455 F27850 F29608 F27206 |
| | | F18418 F31459 F18564 F33496 F16376 F29740 F29843 F29904 F29866 |
| | | F19135 AW276602 T40337 F24835 F34672 F26474 AI926215 |
| | | AA464185 F18217 N26193 AL043256 T41197 F33055 F00386 F29500 F34191 F33297 |
| | | AI937207 F22724 F15909 F26232 F18889 AA318627 F29085 AA872104 F17509 F23373 |
| | | F15660 F17552 F17412 F16863 F34033 F21515 F17364 F18383 F16546 F17561 |
| | | F17260 AA292000 F15723 T47438 F16798 F18046 F18319 F17978 F17566 |
| | | F34230 F33258 F20860 F17998 AI695701 |
| 118600 | genbank_N69222 | N69222 |
| 118952 | genbank_N92966 | N92966 |
| 120873 | genbank_AA358015 | AA358015 |
| 113702 | genbank_T97307 | T97307 |
| 129982 | 221_267 | Z14221 AW381862 M97920 AW401444 Z66542 M29470 AW406502 X61011 M34024 |
| | | AA327072 Z14166 Z14167 Z14165 AW403806 Z14200 AA383972 Z14205 |
| | | Z14201 M18513 Z14202 AW403684 X14584 AF062221 U43760 X65892 X65883 X62107 |
| | | Z80847 X65885 X65893 AF062142 X65891 X17675 Z47274 Z47277 Z47276 X65888 Z47275 |
| | | X62109 AF062140 L01278 AF062134 AF062139 X81723 Z80840 X81733 X81743 X81744 |
| | | X81732 Z80843 AW402942 AW403516 X65919 AF062 |
| | | 190 AF062177 AF062222 AF062115 Z47240 AF062263 AF062261 AF062223 |
| | | AF062211 Z47238 AW401714 AW404008 AW404991 AF062280 M99595 Z47214 |
| | | Z47232 Z47218 M26995 AF062184 X65895 L38433 X81731 Z11946 Z47226 AF062205 AF174012 |
| | | L01276 AF062168 AF062136 X81755 X81748 AF174019 AF062285 L06924 L11699 AW402665 |
| | | L09070 L28049 L08090 AW407843 Z14171 AW402944 L08083 AW405627 L33035 |
| | | L26907 M17750 L28052 M17751 AJ239360 U19885 L14821 X56526 AB014341 L12087 L12098 |
| | | U68231 L12184 AF062242 AF022000 U64499 U00570 AI268604 Y15773 X64239 X62969 U00506 |
| | | X73605 X99360 U00577 D83677 AB021539 AF035796 Z33899 U00588 AJ239353 AF174062 |
| | | Z33901 X98899 AF174058 X63080 D83676 AB021529 Z18318 U00488 L01412 |
| | | X81746 U21262 U21272 U00560 AF174060 U00547 U00561 Z18321 S73957 |
| | | M26435 AF115130 AF115117 Z92896 U21254 Z92895 AF115112 AF062290 T28938 Y09386 |
| | | AF174067 U27189 Y09384 U77373 AF174057 M17749 X69692 AF174038 U64478 U64486 |
| | | AF174098 X99365 AF174077 AF174083 AF174089 AF015133 AF127792 AJ006171 U00510 |
| | | U58144 AF004323 AF115109 AF004324 AF015130 AF090414 AF090418 AB021536 AB021519 |
| | | Z96957 AF021986 Z92898 Z96956 L34164 AF062251 AF052524 U00549 U00541 D83687 |
| | | AF052522 AF087420 AF052520 AF022011 L43085 U00536 L19915 L43083 AF090420 |
| | | AB021530 AB021534 Z96955 Z96954 Z33903 X62964 AF103282 U00509 AF062298 AF062289 |
| | | AF062307 AW408326 L33037 L04337 L04328 X81742 L04336 L03385 AF032360 S56184 |
| | | AF062191 AF191092 AI906954 AF103184 L04343 AW364860 AF001424 AF103163 Z98717 |
| | | AF103143 AJ008250 L04323 AF103321 AF103335 L04342 L03818 L03817 AW404978 |
| | | AW403690 U86801 AF103150 AJ010334 AF035027 AJ007327 AF103115 AF017458 AJ008207 |
| | | AJ008183 AJ008196 AJ008241 AJ008208 AF103210 AF068668 AF068670 AF068671 AF068664 |
| | | AF068669 AF068666 AF068665 AF068672 AW371244 AW403670 AW408074 AW404575 |
| | | AW362153 AW403803 AW406702 AW351514 D78345 T29140 J00231 NM_002179 |
| | | AW405146 AA301091 X04646 H64660 AW402990 AW406534 T93007 |
| | | AI857980 AW368899 AI905833 AW406586 AA482084 AI872299 AA715266 |
| | | AW404328 AI831674 AI709348 AA603112 AW514864 AA485775 H64492 |
| | | AW404789 AA487630 AA715498 AA295885 T27613 T98113 |
| 115763 | genbank_AA421560 | AA421560 |
| 124357 | genbank_N22401 | N22401 |
| 108733 | 504187_1 | AA121022 AA126422 |
| 101544 | entrez_M31169 | M31169 |
| 124447 | genbank_N48000 | N48000 |
| 124677 | genbank_R01073 | R01073 |
| 124777 | genbank_R41933 | R41933 |
| 119302 | genbank_T25725 | T25725 |
| 103680 | entrez_Z93784 | Z93784 |
| 135029 | H58818_at | H58818 |
| 112253 | genbank_R51818 | R51818 |
| 322175 | 46877_1 | AF085975 H53458 H53459 |
| 323011 | 139750_1 | AA580288 AA315655 AA133031 AA377748 |
| 322975 | 1510563_1 | C16391 C16413 |
| 317210 | 211994_1 | AW881145 AA490718 M85637 AA304575 T06067 AA331991 |
| 323817 | 233566_1 | AA410943 AW948953 AA334202 AA332882 |
| 309583 | 1046029_−2 | AW170035 |
| 324961 | 376239_1 | AA613792 AW182329 T05304 AW858385 |
| 303502 | 325188_1 | BE174240 AA488528 AL042253 |
| 320133 | 447553_1 | BE151746 BE336853 D63271 T94955 AA774994 |
| 311935 | 174129_1 | AA216387 T63548 AA228676 |
| 321050 | 502195_1 | C05928 AW393497 |
| 319977 | 345248_1 | AA534222 AA632632 T81234 |
| 312772 | 4380_7 | AW962299 AA310349 AW962294 H63791 H63751 |
| 321354 | 116028_−2 | AA078493 |
| 336512 | CH22_3941FG_834_7_LINK_DJ | |
| 336558 | CH22_3992FG_842_3_LINK_DJ | |
| 336560 | CH22_3994FG_842_5_LINK_DJ | |
| 329367 | c_x_hs | |
| 329373 | c_x_hs | |
| 336676 | CH22_4154FG_43_4_{—} | |
| 338008 | CH22_6490FG_LINK_EM: AC00 | |
| 338057 | CH22_6558FG_LINK_EM: AC00 | |
| 329655 | c14_p2 | |
| 336959 | CH22_4764FG_367_13_{—} | |
| 329899 | c15_p2 | |
| 329960 | c16_p2 | |
| 338410 | CH22_7067FG_LINK_EM: AC00 | |
| 338451 | CH22_7124FG_LINK_EM: AC00 | |
| 338588 | CH22_7331FG_LINK_EM: AC00 | |
| 338665 | CH22_7438FG_LINK_EM: AC00 | |
| 338689 | CH22_7464FG_LINK_EM: AC00 | |
| 308677 | AI761173 | |
| 338832 | CH22_7678FG_LINK_DJ246D7 | |
| 338980 | CH22_7859FG_LINK_DA59H18 | |
| 333009 | CH22_233FG_61_1_LINK_EM: A | |
| 333010 | CH22_234FG_61_2_LINK_EM: A | |
| 333013 | CH22_237FG_61_5_LINK_EM: A | |
| 308981 | AI873242 | |
| 308995 | AI880172 | |
| 333108 | CH22_336FG_79_14_LINK_EM: | |
| 333139 | CH22_368FG_83_16_LINK_EM: | |
| 333254 | CH22_495FG_118_2_LINK_EM: | |
| 333305 | CH22_550FG_137_2_LINK_EM: | |
| 333343 | CH22_589FG_139_12_LINK_EM | |
| 333388 | CH22_634FG_144_3_LINK_EM: | |
| 326213 | c17_hs | |
| 333456 | CH22_706FG_157_5_LINK_EM: | |
| 333459 | CH22_709FG_157_8_LINK_EM: | |
| 333517 | CH22_773FG_173_2_LINK_EM: | |
| 333585 | CH22_846FG_203_4_LINK_EM: | |
| 333679 | CH22_941FG_247_6_LINK_EM: | |
| 326474 | c19_hs | |
| 333743 | CH22_1009FG_264_1_LINK_EM | |
| 333758 | CH22_1024FG_268_1_LINK_EM | |
| 333767 | CH22_1034FG_271_6_LINK_EM | |
| 333768 | CH22_1035FG_271_7_LINK_EM | |
| 333769 | CH22_1036FG_271_8_LINK_EM | |
| 333795 | CH22_1063FG_275_1_LINK_EM | |
| 333796 | CH22_1065FG_275_3_LINK_EM | |
| 335004 | CH22_2326FG_472_8_LINK_EM | |
| 333892 | CH22_1163FG_292_14_LINK_E | |
| 335115 | CH22_2447FG_496_2_LINK_EM | |
| 333904 | CH22_1176FG_294_2_LINK_EM | |
| 333905 | CH22_1177FG_294_3_LINK_EM | |
| 333921 | CH22_1194FG_296_12_LINK_E | |
| 333968 | CH22_1245FG_307_4_LINK_EM | |
| 328059 | c_6_hs | |
| 335287 | CH22_2629FG_526_11_LINK_E | |
| 326816 | c20_hs | |
| 326817 | c20_hs | |
| 335342 | CH22_2689FG_536_1_LINK_EM | |
| 335491 | CH22_2843FG_570_23_LINK_E | |
| 335495 | CH22_2847FG_570_28_LINK_E | |
| 335498 | CH22_2850FG_571_7_LINK_EM | |
| 328304 | c_7_hs | |
| 305453 | AA738110 | |
| 335544 | CH22_2899FG_576_5_LINK_EM | |
| 335610 | CH22_2969FG_583_4_LINK_EM | |
| 335653 | CH22_3013FG_590_4_LINK_EM | |
| 335682 | CH22_3043FG_595_2_LINK_EM | |
| 335687 | CH22_3048FG_596_2_LINK_EM | |
| 328492 | c_7_hs | |
| 335755 | CH22_3122FG_604_4_LINK_EM | |
| 335782 | CH22_3151FG_609_4_LINK_EM | |
| 335791 | CH22_3160FG_611_7_LINK_EM | |
| 335809 | CH22_3181FG_617_6_LINK_EM | |
| 335822 | CH22_3195FG_619_7_LINK_EM | |
| 335823 | CH22_3196FG_619_8_LINK_EM | |
| 335824 | CH22_3197FG_619_11_LINK_E | |
| 335825 | CH22_3198FG_619_12_LINK_E | |
| 335895 | CH22_3272FG_635_3_LINK_EM | |
| 335917 | CH22_3294FG_636_13_LINK_E | |
| 335920 | CH22_3297FG_636_16_LINK_E | |
| 305898 | AA872838 | |
| 305913 | AA876109 | |
| 305950 | AA884479 | |
| 328857 | c_7_hs | |
| 330084 | c19_p2 | |
| 337968 | CH22_6419FG_LINK_EM: AC00 | |
| 309177 | AI951118 | |
| 309198 | AI955915 | |
| 309226 | AI969897 | |
| 339352 | CH22_8317FG_LINK_BA354I1 | |
| 309279 | AI990102 | |
| 339373 | CH22_8348FG_LINK_BA232E1 | |
| 325622 | c14_hs | |
| 334102 | CH22_1380FG_327_60_LINK_E | |
| 332927 | CH22_148FG_38_1_LINK_C20H | |
| 332929 | CH22_150FG_38_3_LINK_C20H | |
| 332930 | CH22_151FG_38_4_LINK_C20H | |
| 332955 | CH22_179FG_48_12_LINK_EM: | |
| 332958 | CH22_182FG_48_15_LINK_EM: | |
| 332961 | CH22_185FG_48_18_LINK_EM: | |
| 332983 | CH22_207FG_54_5_LINK_EM: A | |
| 334222 | CH22_1506FG_360_3_LINK_EM | |
| 334223 | CH22_1507FG_360_4_LINK_EM | |
| 334264 | CH22_1551FG_367_15_LINK_E | |
| 327110 | c21_hs | |
| 334343 | CH22_1636FG_375_25_LINK_E | |
| 334360 | CH22_1654FG_378_5_LINK_EM | |
| 327196 | c_1_hs | |
| 327283 | c_1_hs | |
| 327313 | c_1_hs | |
| 304465 | AA421948 | |
| 304507 | AA456426 | |
| 327450 | c_2_hs | |
| 304591 | AA505702 | |
| 304601 | AA507875 | |
| 304659 | AA533185 | |
| 334784 | CH22_2096FG_432_9_LINK_EM | |
| 334789 | CH22_2101FG_432_14_LINK_E | |
| 334794 | CH22_2106FG_434_2_LINK_EM | |
| 336035 | CH22_3420FG_678_6_LINK_DJ | |
| 336042 | CH22_3427FG_679_4_LINK_DJ | |
| 336093 | CH22_3481FG_691_2_LINK_DJ | |
| 336096 | CH22_3484FG_691_5_LINK_DJ | |
| 334889 | CH22_2206FG_452_3_LINK_EM | |
| 336150 | CH22_3540FG_706_6_LINK_DA | |
| 336152 | CH22_3543FG_706_9_LINK_DA | |
| 336416 | CH22_3833FG_823_38_LINK_B | |
| 336444 | CH22_3864FG_827_10_LINK_D | |
| 336449 | CH22_3870FG_829_6_LINK_DJ | |
| 336471 | CH22_3894FG_829_30_LINK_D | |

Pkey: Unique Eos probeset identifier number
CAT number: Gene cluster number
Accession: Genbank accession numbers

TABLE 13B
Table 13B shows the genomic positioning for
those pkeys lacking unigene ID's and
accession numbers in Table 13B. For each
predicted exon, we have listed the genomic
sequence source used for prediction. Nucleotide
locations of each predicted exon are also listed.

| Pkey | Ref | Strand | Nt_position |
|---|---|---|---|
| 332955 | Dunham, I. et.al. | Plus | 2508896-2508992 |
| 332958 | Dunham, I. et.al. | Plus | 2516164-2516310 |
| 332961 | Dunham, I. et.al. | Plus | 2521424-2521555 |
| 333139 | Dunham, I. et.al. | Plus | 3369495-3369571 |
| 333254 | Dunham, I. et.al. | Plus | 2521424-2521555 |
| 333305 | Dunham, I. et.al. | Plus | 4630388-4630645 |
| 333388 | Dunham, I. et.al. | Plus | 4913749-4913805 |
| 333517 | Dunham, I. et.al. | Plus | 5570729-5570925 |
| 333585 | Dunham, I. et.al. | Plus | 6234778-6234894 |
| 333679 | Dunham, I. et.al. | Plus | 7068795-7068896 |
| 333767 | Dunham, I. et.al. | Plus | 7694407-7694623 |
| 333768 | Dunham, I. et.al. | Plus | 7695440-7695697 |
| 333769 | Dunham, I. et.al. | Plus | 7696625-7696707 |
| 333795 | Dunham, I. et.al. | Plus | 7807688-7807795 |
| 333796 | Dunham, I. et.al. | Plus | 7808253-7808319 |
| 333892 | Dunham, I. et.al. | Plus | 8156825-8157001 |
| 333921 | Dunham, I. et.al. | Plus | 8380325-8380441 |
| 333968 | Dunham, I. et.al. | Plus | 8681004-8681241 |
| 334102 | Dunham, I. et.al. | Plus | 9995140-9996373 |
| 334264 | Dunham, I. et.al. | Plus | 13234447-13234544 |
| 334343 | Dunham, I. et.al. | Plus | 13655828-13656307 |
| 334794 | Dunham, I. et.al. | Plus | 16374312-16374458 |
| 334889 | Dunham, I. et.al. | Plus | 19286024-19286515 |
| 335287 | Dunham, I. et.al. | Plus | 22299047-22299299 |
| 335491 | Dunham, I. et.al. | Plus | 24128651-24128827 |
| 335495 | Dunham, I. et.al. | Plus | 24140688-24140872 |
| 335498 | Dunham, I. et.al. | Plus | 24172082-24172161 |
| 335653 | Dunham, I. et.al. | Plus | 25329710-25329802 |
| 335687 | Dunham, I. et.al. | Plus | 25445952-25446064 |
| 335809 | Dunham, I. et.al. | Plus | 26310772-26310909 |
| 335822 | Dunham, I. et.al. | Plus | 26364087-26364196 |
| 335823 | Dunham, I. et.al. | Plus | 26365925-26366004 |
| 335824 | Dunham, I. et.al. | Plus | 26376860-26376942 |
| 335825 | Dunham, I. et.al. | Plus | 26378175-26378268 |
| 336035 | Dunham, I. et.al. | Plus | 29016748-29017410 |
| 336093 | Dunham, I. et.al. | Plus | 29556922-29557002 |
| 336096 | Dunham, I. et.al. | Plus | 29578878-29579047 |
| 336444 | Dunham, I. et.al. | Plus | 34190585-34190718 |
| 336959 | Dunham, I. et.al. | Plus | 13233040-13233126 |
| 338008 | Dunham, I. et.al. | Plus | 7697068-7697236 |
| 338057 | Dunham, I. et.al. | Plus | 8526397-8526522 |
| 338410 | Dunham, I. et.al. | Plus | 19292807-19292916 |
| 338588 | Dunham, I. et.al. | Plus | 22896767-22896920 |
| 338665 | Dunham, I. et.al. | Plus | 24472654-24472853 |
| 338832 | Dunham, I. et.al. | Plus | 27775128-27775290 |
| 338980 | Dunham, I. et.al. | Plus | 29896789-29896874 |
| 339352 | Dunham, I. et.al. | Plus | 33544784-33545121 |
| 332929 | Dunham, I. et.al. | Minus | 2020758-2020664 |
| 332930 | Dunham, I. et.al. | Minus | 2022565-2022497 |
| 332983 | Dunham, I. et.al. | Minus | 2631933-2631797 |
| 333009 | Dunham, I. et.al. | Minus | 2766043-2765856 |
| 333010 | Dunham, I. et.al. | Minus | 2766207-2766119 |
| 333013 | Dunham, I. et.al. | Minus | 2772278-2772039 |
| 333108 | Dunham, I. et.al. | Minus | 3240494-3240389 |
| 333343 | Dunham, I. et.al. | Minus | 4692886-4692753 |
| 333456 | Dunham, I. et.al. | Minus | 2631933-2631797 |
| 333459 | Dunham, I. et.al. | Minus | 5144548-5144344 |
| 333743 | Dunham, I. et.al. | Minus | 7573218-7573060 |
| 333758 | Dunham, I. et.al. | Minus | 7666413-7666091 |
| 333904 | Dunham, I. et.al. | Minus | 8217374-8217261 |
| 333905 | Dunham, I. et.al. | Minus | 8217796-8217670 |
| 334222 | Dunham, I. et.al. | Minus | 12732417-12732289 |
| 334223 | Dunham, I. et.al. | Minus | 12734365-12734269 |
| 334360 | Dunham, I. et.al. | Minus | 13728850-13728751 |
| 334784 | Dunham, I. et.al. | Minus | 16294548-16294360 |
| 334789 | Dunham, I. et.al. | Minus | 16306095-16305996 |
| 335004 | Dunham, I. et.al. | Minus | 20581911-20581794 |
| 335115 | Dunham, I. et.al. | Minus | 21388250-21388146 |
| 335342 | Dunham, I. et.al. | Minus | 22597448-22597284 |
| 335544 | Dunham, I. et.al. | Minus | 24650505-24650403 |
| 335610 | Dunham, I. et.al. | Minus | 25068943-25068841 |
| 335682 | Dunham, I. et.al. | Minus | 25421215-25421093 |
| 335755 | Dunham, I. et.al. | Minus | 25763806-25763747 |
| 335782 | Dunham, I. et.al. | Minus | 25908578-25908440 |
| 335791 | Dunham, I. et.al. | Minus | 25948563-25948411 |
| 335895 | Dunham, I. et.al. | Minus | 26975307-26975239 |
| 335917 | Dunham, I. et.al. | Minus | 27028481-27028377 |
| 335920 | Dunham, I. et.al. | Minus | 27034927-27034811 |
| 336042 | Dunham, I. et.al. | Minus | 29041694-29041500 |
| 336150 | Dunham, I. et.al. | Minus | 30150423-30150256 |
| 336152 | Dunham, I. et.al. | Minus | 30156053-30155870 |
| 336416 | Dunham, I. et.al. | Minus | 34047408-34047311 |
| 336449 | Dunham, I. et.al. | Minus | 34204707-34204577 |
| 336471 | Dunham, I. et.al. | Minus | 34215091-34214978 |
| 336512 | Dunham, I. et.al. | Minus | 34278373-34278275 |
| 336558 | Dunham, I. et.al. | Minus | 34375825-34375698 |
| 336560 | Dunham, I. et.al. | Minus | 34376814-34376596 |
| 336676 | Dunham, I. et.al. | Minus | 2022565-2022497 |
| 337968 | Dunham, I. et.al. | Minus | 7095797-7095680 |
| 338451 | Dunham, I. et.al. | Minus | 20174286-20174193 |
| 338689 | Dunham, I. et.al. | Minus | 24893073-24892972 |
| 339373 | Dunham, I. et.al. | Minus | 33860127-33860047 |
| 325622 | 5867000 | Plus | 69994-70075 |
| 329655 | 6448516 | Minus | 35565-35843 |
| 329899 | 6563505 | Minus | 111058-111783 |
| 329960 | 5091594 | Minus | 1031-1162 |
| 326213 | 5867224 | Minus | 60751-60927 |
| 326474 | 5867405 | Plus | 16995-18101 |
| 330084 | 6015302 | Minus | 57019-59337 |
| 326816 | 6552458 | Plus | 198354-198436 |
| 326817 | 6552458 | Plus | 199909-200001 |
| 327110 | 6117842 | Plus | 94608-94785 |
| 327196 | 5867446 | Plus | 180921-181333 |
| 327283 | 5867478 | Minus | 567-962 |
| 327313 | 5867501 | Minus | 89734-89838 |
| 327450 | 5867766 | Minus | 47928-48076 |
| 328059 | 6117819 | Plus | 37052-37204 |
| 328492 | 5868455 | Minus | 46094-46241 |
| 328304 | 6004478 | Minus | 3884-3952 |
| 328857 | 6381927 | Minus | 80557-81051 |
| 329367 | 5868842 | Minus | 87201-87587 |
| 329373 | 6682537 | Minus | 38950-39301 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 14
Table 2 from BRCA 001-5 US
Table 14, a subset of table 13, depicts a preferred group of
genes highly upregulated in breast cancer cells.

| Pkey | ExAccn | UniGene ID | Unigene Title | R1 |
|---|---|---|---|---|
| 100038 | M97935 | | AFFX control: STAT1 | 16.7 |
| 100114 | D00596 | Hs.82962 | thymidylate synthetase | 15.9 |
| 100975 | J02923 | Hs.76506 | lymphocyte cytosolic protein 1 (L-plastin) | 30.1 |
| 101031 | J05070 | Hs.151738 | matrix metalloproteinase 9 (gelatinase B; 9 | 37.2 |
| 101104 | L07615 | Hs.169266 | Human neuropeptideY receptor Y1 (NPYY | 18.3 |
| 101143 | L12723 | Hs.90093 | heat shock 70 kD protein 4 | 17.4 |
| 101332 | L47276 | Hs.156346 | Homo sapiens (cell line HL-6) alpha topois | 18.9 |
| 101378 | M13755 | Hs.833 | interferon-stimulated protein; 15 kDa | 18.1 |
| 101809 | M86849 | Hs.323733 | Homo sapiens connexin 26 (GJB2) mRNA | 22.5 |
| 102618 | U65932 | Hs.81071 | extracellular matrix protein 1 | 23.2 |
| 102721 | U79241 | Hs.118666 | Human clone 23759 mRNA; partial cds | 15 |
| 102817 | U90904 | Hs.83724 | Human clone 23773 mRNA sequence | 15.2 |
| 102907 | X06985 | Hs.202833 | heme oxygenase (decycling) 1 | 22.7 |
| 102985 | X17644 | Hs.2707 | G1 to S phase transition 1 | 20.6 |
| 103060 | X57766 | Hs.155324 | matrix metalloproteinase 11 (stromelysin 3 | 17.8 |
| 103180 | X69433 | Hs.5337 | isocitrate dehydrogenase 2 (NADP +); mito | 18.9 |
| 103206 | X72755 | Hs.77367 | monokine induced by gamma interferon | 15.1 |
| 103821 | AA157623 | Hs.198793 | KIAA0750 gene product | 23.3 |
| 104115 | AA428090 | Hs.26102 | ESTs | 28.7 |
| 104667 | AA007234 | Hs.30098 | ESTs | 16.6 |
| 105186 | AA191512 | Hs.28005 | Homo sapiens mRNA; cDNA DKFZp564G | 19.3 |
| 106103 | AA421104 | Hs.12094 | ESTs | 15.4 |
| 107151 | AA621169 | Hs.8687 | ESTs | 19 |
| 109415 | AA227219 | Hs.110826 | trinucleotide repeat containing 9 | 20.1 |
| 110189 | H20543 | Hs.6278 | DKFZP586B1621 protein | 16.6 |
| 110561 | H59617 | Hs.5199 | ESTs; Weakly similar to UBIQUITIN-CON | 19.5 |
| 110734 | H98714 | Hs.24131 | ESTs | 30.2 |
| 110915 | N46252 | Hs.29724 | ESTs | 23.2 |
| 111179 | N67239 | Hs.10760 | ESTs | 37 |
| 111357 | N91023 | Hs.87128 | ESTs | 15 |
| 112134 | R46025 | Hs.7413 | ESTs | 17.4 |
| 113970 | W86748 | Hs.8109 | ESTs | 15 |
| 114124 | Z38595 | Hs.125019 | ESTs; Highly similar to KIAA0886 protein | 22 |
| 114292 | Z40715 | Hs.184641 | delta-6 fatty acid desaturase | 19.4 |
| 114901 | AA236276 | Hs.196437 | ESTs; Weakly similar to R26660_1; partial | 16.9 |
| 114965 | AA250737 | Hs.72472 | ESTs | 35.1 |
| 115652 | AA405098 | Hs.38178 | ESTs | 16.1 |
| 115875 | AA433943 | Hs.43946 | ESTs; Weakly similar to Weak similarity t | 33.5 |
| 116790 | H29532 | Hs.101174 | microtubule-associated protein tau | 22.2 |
| 116921 | H72948 | Hs.821 | biglycan | 20.7 |
| 117412 | N26722 | Hs.42645 | ESTs | 18.1 |
| 120241 | Z41815 | Hs.65946 | ESTs | 15.6 |
| 120325 | AA195651 | Hs.104106 | ESTs | 15.2 |
| 121596 | AA416740 | Hs.174104 | ESTs | 22.6 |
| 123619 | AA609200 | | ESTs | 23.1 |
| 124006 | D60302 | Hs.270016 | ESTs | 20.6 |
| 125852 | H09290 | Hs.76550 | Homo sapiens mRNA; cDNA DKFZp564B | 25.9 |
| 126160 | N90960 | Hs.265398 | ESTs; Weakly similar to transformation-rel | 16.4 |
| 127677 | AA916752 | Hs.264190 | ESTs; Highly similar to MEM3 [M. muscul | 17.3 |
| 128595 | U31875 | Hs.152677 | short-chain alcohol dehydrogenase family m | 27.1 |
| 128717 | T30617 | Hs.104222 | Homo sapiens mRNA; cDNA DKFZp566L | 24.5 |
| 129124 | AA234530 | Hs.108802 | N-ethylmaleimide-sensitive factor | 20.7 |
| 129366 | H18027 | Hs.184697 | plexin C1 | 18.2 |
| 130455 | X17059 | Hs.155956 | N-acetyltransferase 1 (arylamine N-acetylt | 26.4 |
| 130604 | X03635 | Hs.1657 | estrogen receptor 1 | 39.9 |
| 130913 | W03592 | Hs.21198 | translocase of outer mitochondrial membra | 20.9 |
| 130944 | M97935 | Hs.21486 | signal transducer and activator of transcript | 18.8 |
| 131472 | AA608962 | Hs.27258 | calcyclin binding protein | 18.1 |
| 131562 | U90551 | Hs.28777 | H2A histone family; member L | 18.8 |
| 132180 | AA405569 | Hs.418 | fibroblast activation protein; alpha; seprase | 15.4 |
| 132406 | F09979 | Hs.4774 | ESTs | 15 |
| 132465 | AA047896 | Hs.49169 | ESTs | 15.4 |
| 132994 | AA505133 | Hs.279905 | solute carrier family 2 (facilitated glucose t | 26.4 |
| 133294 | R79723 | Hs.69997 | zinc finger protein 238 | 30.4 |
| 133634 | U24166 | Hs.234279 | microtubule-associated protein; RP/EB fam | 15.2 |
| 134374 | D62633 | Hs.8236 | ESTs | 15.2 |
| 134405 | J04177 | Hs.82772 | collagen; type XI; alpha 1 | 15.3 |
| 134470 | X54942 | Hs.83758 | CDC28 protein kinase 2 | 20.3 |
| 134495 | D63477 | Hs.84087 | KIAA0143 protein | 16.1 |
| 134714 | U89922 | Hs.890 | lymphotoxin beta (TNF superfamily; memb | 35.7 |
| 135237 | AA454930 | Hs.9691 | ESTs | 19.5 |
| 301884 | AA312082 | Hs.105445 | GDNF family receptor alpha 1 | 20.7 |
| 302276 | NM_004448 | Hs.323910 | EST cluster (not in UniGene) with exon hit | 21.6 |
| 302290 | AL117607 | Hs.175563 | Homo sapiens mRNA; cDNA DKFZp564N | 41.4 |
| 309177 | AI951118 | | EST singleton (not in UniGene) with exon | 24.3 |
| 309583 | AW170035 | | EST | 64.5 |
| 310438 | AW022192 | Hs.200197 | ESTs | 39.1 |
| 311166 | AI821294 | Hs.118599 | ESTs | 24.1 |
| 312153 | AA759250 | Hs.153028 | cytochrome b-561 | 27.1 |
| 313915 | AI969390 | Hs.163443 | ESTs | 27.1 |
| 314506 | AA833655 | Hs.206868 | ESTs | 27.8 |
| 314558 | AI873274 | Hs.190721 | ESTs | 22.5 |
| 314691 | AW207206 | Hs.136319 | ESTs | 21.4 |
| 314943 | AI476797 | Hs.184572 | cell division cycle 2; G1 to S and G2 to M | 18.4 |
| 315196 | AA972756 | Hs.44898 | ESTs | 28.8 |
| 316177 | AI908272 | Hs.293102 | EST cluster (not in UniGene) | 32.6 |
| 318073 | AW167087 | Hs.131562 | ESTs | 15.7 |
| 318662 | AI285898 | Hs.294014 | ESTs | 16.3 |
| 318740 | NM_002543 | Hs.77729 | EST cluster (not in UniGene) | 21.3 |
| 318744 | AI793124 | Hs.144479 | ESTs | 35 |
| 319668 | NM_002731 | Hs.87773 | EST cluster (not in UniGene) | 25.4 |
| 320074 | AA321166 | Hs.278233 | EST cluster (not in UniGene) | 16.7 |
| 320211 | AL039402 | Hs.125783 | DEME-6 protein | 24.3 |
| 320727 | U96044 | Hs.181125 | EST cluster (not in UniGene) | 15.3 |
| 322818 | AW043782 | Hs.293616 | ESTs | 21 |
| 322882 | AW248508 | Hs.279727 | DiGeorge syndrome critical region gene 2 | 15.3 |
| 324261 | AL044891 | Hs.269350 | EST cluster (not in UniGene) | 50.1 |
| 324432 | AA464510 | Hs.152812 | EST cluster (not in UniGene) | 16.7 |
| 324603 | AW016378 | Hs.292934 | ESTs | 23.1 |
| 324620 | AA448021 | Hs.94109 | EST cluster (not in UniGene) | 21.2 |
| 324988 | T06997 | Hs.121028 | EST cluster (not in UniGene) | 24.5 |
| 330388 | X03363 | | HER2 receptor tyrosine kinase (c-erbB-2; E | 17.7 |
| 330486 | M13755 | Hs.833 | interferon-stimulated protein; 15 kDa | 67 |
| 330814 | AA015730 | Hs.265398 | ESTs; Weakly similar to transformation-rel | 44.1 |
| 331145 | R72427 | Hs.129873 | ESTs; Weakly similar to CYTOCHROME | 41.9 |
| 331306 | AA252079 | Hs.63931 | dachshund (Drosophila) homolog | 15.1 |
| 331890 | AA432166 | Hs.3577 | succinate dehydrogenase complex; subunit | 24.3 |
| 332526 | AA281753 | Hs.77515 | inositol 1; 4; 5-triphosphate receptor; type 3 | 19 |
| 332532 | N63192 | Hs.1892 | EST; Highly similar to PHENYLETHANO | 15.3 |
| 332694 | AA262768 | Hs.243901 | KIAA1067 protein | 15.2 |
| 332958 | | | CH22_FGENES.48_15 | 17.8 |
| 333769 | | | CH22_FGENES.271_8 | 48.3 |
| 333968 | | | CH22_FGENES.307_4 | 15.9 |
| 334223 | | | CH22_FGENES.360_4 | 33.5 |
| 334264 | | | CH22_FGENES.367_15 | 18.5 |
| 335791 | | | CH22_FGENES.611_7 | 27.3 |
| 336512 | | | CH22_FGENES.834_7 | 21.4 |
| 338008 | | | CH22_EM: AC005500.GENSCAN.127-9 | 15.2 |

Pkey: Unique Eos probeset identifier number
ExAccn: Exemplar Accession number, Genbank accession number
UnigeneID: Unigene number
Unigene Title: Unigene gene title
R1: Ratio of tumor to normal breast tissue

TABLE 14A
Table 14A shows the accession numbers for those
pkeys lacking unigeneID's for Table 14.
For each probeset, we have listed the gene cluster
number from which the oligonucleotides were designed.
Gene clusters were compiled using sequences derived
from Genbank ESTs and mRNAs. These sequences
were clustered based on sequence similarity using
Clustering and Alignment Tools (DoubleTwist, Oakland
California). The Genbank accession numbers
for sequences comprising each cluster are listed
in the “Accession” column.

| Pkey | CAT number | Accession |
|---|---|---|
| 309583 | 1046029_−2 | AW170035 |
| 336512 | CH22_3941FG_834_7_LINK_DJ | |
| 338008 | CH22_6490FG_LINK_EM: AC00 | |
| 333769 | CH22_1036FG_271_8_LINK_EM | |
| 333968 | CH22_1245FG_307_4_LINK_EM | |
| 335791 | CH22_3160FG_611_7_LINK_EM | |
| 309177 | AI951118 | |
| 332958 | CH22_182FG_48_15_LINK_EM: | |
| 334223 | CH22_1507FG_360_4_LINK_EM | |
| 334264 | CH22_1551FG_367_15_LINK_E | |
| 123619 | 371681_1 | AA602964 AA609200 |

Pkey: Unique Eos probeset identifier number
CAT number: Gene cluster number
Accession: Genbank accession numbers

TABLE 14B
Table 14B shows the genomic positioning for those
pkeys lacking unigene ID's and accession numbers
in Table 14. For each predicted exon, we have listed
the genomic sequence source used for prediction.
Nucleotide locations of each predicted exon are also
listed.

| Pkey | Ref | Strand | Nt_position |
|---|---|---|---|
| 332958 | Dunham, I. et.al. | Plus | 2516164-2516310 |
| 333769 | Dunham, I. et.al. | Plus | 7696625-7696707 |
| 333968 | Dunham, I. et.al. | Plus | 8681004-8681241 |
| 334264 | Dunham, I. et.al. | Plus | 13234447-13234544 |
| 338008 | Dunham, I. et.al. | Plus | 7697068-7697236 |
| 334223 | Dunham, I. et.al. | Minus | 12734365-12734269 |
| 335791 | Dunham, I. et.al. | Minus | 25948563-25948411 |
| 336512 | Dunham, I. et.al. | Minus | 34278373-34278275 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 15
Table 3 from BRCA 001-5 US
Table 15 shows genes downregulated in breast cancer cells.

| Pkey | ExAccn | UniGene ID | Unigene Title | R1 |
|---|---|---|---|---|
| 100115 | D00632 | Hs.172153 | glutathione peroxidase 3 (plasma) | 1.7 |
| 100499 | TIGR: HT1428 | Hs.283108 | Globin, Beta | 1.5 |
| 100502 | TIGR: HT1496 | Hs.169228 | Adrenal-Specific Protein Pg2 | 2.3 |
| 100815 | TIGR: HT4268 | Hs.9739 | L-Glycerol-3-Phosphate: Nad + Oxidoreduct | 1.7 |
| 101125 | L10373 | Hs.82749 | transmembrane 4 superfamily member 2 | 1.5 |
| 101367 | M12963 | Hs.4 | alcohol dehydrogenase 1 (class I); alpha po | 2.9 |
| 101397 | M15856 | Hs.180878 | lipoprotein lipase | 1.6 |
| 101883 | M98399 | Hs.75613 | CD36 antigen (collagen type I receptor; thr | 1.6 |
| 102227 | U25138 | Hs.93841 | potassium large conductance calcium-activ | 1.6 |
| 102857 | X00129 | Hs.76461 | retinol-binding protein 4; interstitial | 3 |
| 103211 | X73079 | Hs.288579 | polymeric immunoglobulin receptor | 1.8 |
| 103496 | Y09267 | Hs.132821 | flavin containing monooxygenase 2 | 1.5 |
| 103562 | Z21966 | Hs.2815 | POU domain; class 6; transcription factor 1 | 1.8 |
| 104672 | AA007629 | | glycerol-3-phosphate dehydrogenase 1 (sol | 2.4 |
| 105083 | AA146619 | Hs.18791 | ESTs; Weakly similar to CALCIUM-BIND | 1.7 |
| 105138 | AA164519 | Hs.15248 | ESTs | 1.5 |
| 106075 | AA417915 | Hs.25930 | ESTs | 1.5 |
| 106870 | AA487576 | Hs.26530 | serum deprivation response (phosphatidyls | 1.6 |
| 107099 | AA609645 | Hs.211568 | eukaryotic translation initiation factor 4 gam | 2.7 |
| 107616 | AA004901 | Hs.261164 | ESTs | 1.6 |
| 107997 | AA037388 | Hs.82223 | Human DNA sequence from clone 141H5 o | 1.7 |
| 108604 | AA099820 | Hs.49696 | ESTs | 2.4 |
| 111130 | N64265 | Hs.19515 | yz44h12.s1 Morton Fetal Cochlea Homo sa | 1.7 |
| 111837 | R36447 | Hs.24453 | ESTs | 1.6 |
| 112538 | R70255 | | ESTs | 1.9 |
| 112808 | R97970 | Hs.281022 | EST | 1.5 |
| 113086 | T40652 | Hs.209100 | DKFZP434C171 protein | 1.9 |
| 115740 | AA418033 | Hs.283559 | ESTs | 1.6 |
| 115949 | AA443800 | Hs.43125 | ESTs | 2 |
| 115965 | AA446661 | Hs.173233 | ESTs | 2.2 |
| 117224 | N20300 | Hs.218707 | ESTs | 1.7 |
| 117513 | N32174 | Hs.44317 | SRY (sex-determining region Y)-box 10 | 1.7 |
| 119059 | R15436 | Hs.77889 | Friedreich ataxia region gene X123 | 1.7 |
| 119175 | R71792 | Hs.301002 | ESTs; Weakly similar to cell death activato | 2.8 |
| 119359 | T71021 | Hs.285681 | ESTs; Highly similar to WS basic-helix-loo | 1.9 |
| 119798 | W73386 | Hs.249129 | ESTs | 3 |
| 120889 | AA365784 | Hs.97044 | ESTs | 1.6 |
| 121381 | AA405747 | Hs.97984 | ESTs; Weakly similar to WASP-family pro | 1.8 |
| 121750 | AA421184 | Hs.97549 | ESTs | 1.5 |
| 122127 | AA434447 | Hs.106771 | ESTs | 2.5 |
| 122348 | AA443695 | Hs.293410 | ESTs | 2.1 |
| 122485 | AA448300 | Hs.160318 | phospholemman | 1.5 |
| 123443 | AA598841 | Hs.167382 | natriuretic peptide receptor A/guanylate cy | 1.8 |
| 123505 | AA600135 | | ESTs; Moderately similar to !!!! ALU SUB | 1.5 |
| 125284 | W94688 | Hs.103253 | perilipin | 1.7 |
| 126300 | D81972 | | HUM427D08B Human fetal brain (TFujiw | 1.8 |
| 126747 | R72515 | Hs.160318 | phospholemman | 1.6 |
| 127218 | AA309765 | Hs.116017 | ESTs; Weakly similar to KIAA0795 protei | 1.5 |
| 127357 | AA452788 | Hs.75432 | zx39g11.r1 Soares_total_fetus_Nb2HF8_9 | 1.7 |
| 127638 | AA634405 | Hs.122608 | ESTs | 1.5 |
| 128213 | AA972780 | Hs.129194 | ESTs; Weakly similar to !!!! ALU SUBFA | 1.5 |
| 128351 | AI092391 | Hs.134886 | ESTs | 1.5 |
| 128842 | N44757 | Hs.20340 | ESTs | 1.6 |
| 128870 | R71403 | Hs.75309 | eukaryotic translation elongation factor 2 | 1.7 |
| 129146 | AA459944 | Hs.108924 | DKFZP586P1422 protein | 1.5 |
| 129285 | T62068 | Hs.11006 | ESTs | 2.1 |
| 129331 | N93465 | Hs.279772 | ESTs; Highly similar to CGI-38 protein [H | 1.5 |
| 130085 | M62402 | Hs.274313 | insulin-like growth factor binding protein 6 | 1.7 |
| 130400 | M25079 | Hs.283108 | hemoglobin; beta | 1.7 |
| 131267 | AA211776 | Hs.2504 | myomesin 1 (skelemin) (185 kD) | 3.8 |
| 131277 | AA131466 | Hs.23767 | ESTs | 1.9 |
| 131282 | M12272 | Hs.4 | alcohol dehydrogenase 3 (class I); gamma p | 2.2 |
| 131304 | AA295848 | Hs.25475 | aquaporin 7 | 1.7 |
| 131810 | D49487 | Hs.194236 | leptin (murine obesity homolog) | 2.5 |
| 132788 | AA045503 | Hs.56874 | ESTs; Weakly similar to Homo sapiens p2 | 1.6 |
| 132931 | Z41452 | Hs.6090 | deleted in bladder cancer chromosome regi | 1.5 |
| 133120 | X64559 | Hs.65424 | tetranectin (plasminogen-binding protein) | 2 |
| 133314 | U95367 | Hs.70725 | gamma-aminobutyric acid (GABA) A recep | 1.5 |
| 133507 | X74295 | Hs.74369 | integrin; alpha 7 | 1.7 |
| 133601 | S95936 | Hs.284176 | transferrin | 2.3 |
| 133702 | N56898 | Hs.75652 | glutathione S-transferase M5 | 1.9 |
| 134111 | N79674 | Hs.8022 | TU3A protein | 4.6 |
| 134699 | U56814 | Hs.88646 | deoxyribonuclease I-like 3 | 1.5 |
| 134749 | L10955 | Hs.89485 | carbonic anhydrase IV | 1.6 |
| 135173 | M72885 | Hs.95910 | Human G0S2 protein gene; complete cds | 1.9 |
| 300132 | AW027556 | Hs.156286 | ESTs | 1.7 |
| 300732 | AI369956 | Hs.257891 | ESTs | 1.5 |
| 300750 | AA514805 | Hs.293055 | ESTs | 1.8 |
| 301140 | AI807692 | Hs.129129 | ESTs | 1.6 |
| 301396 | AA923549 | Hs.224121 | ESTs | 2.1 |
| 302910 | N77976 | Hs.251577 | hemoglobin; alpha 1 | 1.8 |
| 303798 | V00505 | Hs.36977 | hemoglobin; delta | 1.6 |
| 303831 | T04868 | Hs.46780 | EST cluster (not in UniGene) with exon hit | 1.7 |
| 303844 | U94362 | Hs.58589 | glycogenin 2 | 1.5 |
| 304182 | H91086 | | EST singleton (not in UniGene) with exon | 1.5 |
| 304622 | AA516384 | | EST singleton (not in UniGene) with exon | 1.5 |
| 304682 | AA550994 | | EST singleton (not in UniGene) with exon | 1.7 |
| 305612 | AA782347 | Hs.272572 | EST singleton (not in UniGene) with exon | 1.5 |
| 306193 | AA923457 | | EST singleton (not in UniGene) with exon | 1.5 |
| 307206 | AI192534 | | EST singleton (not in UniGene) with exon | 1.6 |
| 307377 | AI222691 | | EST singleton (not in UniGene) with exon | 1.5 |
| 308023 | AI452732 | Hs.251577 | EST singleton (not in UniGene) with exon | 1.9 |
| 308359 | AI612774 | Hs.79372 | retinoid X receptor, beta | 1.5 |
| 309838 | AW296073 | Hs.255504 | EST | 1.5 |
| 310403 | AI720978 | Hs.148006 | ESTs; Moderately similar to alternatively s | 1.8 |
| 311671 | AW241947 | Hs.232478 | ESTs | 1.6 |
| 311794 | AW238092 | Hs.254759 | ESTs | 2.1 |
| 312082 | T79860 | Hs.118180 | ESTs | 1.9 |
| 312575 | H25237 | Hs.306814 | ESTs | 2.3 |
| 313076 | N49684 | Hs.143040 | ESTs | 1.8 |
| 313283 | W32480 | Hs.157099 | ESTs | 2.2 |
| 313374 | AW328672 | Hs.132760 | ESTs | 1.9 |
| 314701 | AI754634 | Hs.131987 | ESTs | 1.7 |
| 315391 | AA759098 | Hs.192007 | ESTs | 1.8 |
| 315688 | AA680055 | Hs.264885 | ESTs | 1.5 |
| 316249 | AA948612 | Hs.130414 | ESTs | 1.6 |
| 316586 | AI205077 | Hs.294085 | ESTs | 1.7 |
| 316890 | AA837079 | Hs.24647 | ESTs | 1.5 |
| 316983 | AI480204 | Hs.177131 | ESTs | 1.5 |
| 317604 | AI650625 | Hs.300756 | ESTs | 1.6 |
| 317951 | AW206520 | Hs.129621 | ESTs | 1.5 |
| 319400 | W26902 | Hs.154085 | ESTs | 1.7 |
| 320757 | H22654 | Hs.6382 | EST cluster (not in UniGene) | 1.5 |
| 321594 | AA021402 | Hs.11067 | ESTs | 1.7 |
| 322102 | H45589 | | EST cluster (not in UniGene) | 1.5 |
| 322814 | AI824495 | Hs.211038 | ESTs | 2.2 |
| 3022929 | AI365585 | Hs.146246 | ESTs | 2.3 |
| 323831 | AA335715 | Hs.200299 | ESTs | 1.7 |
| 324044 | AL045752 | Hs.22350 | ESTs | 1.8 |
| 324675 | AW014734 | Hs.157969 | ESTs | 2.2 |
| 325272 | | | CH.11_hs gi\|5866902 | 1.5 |
| 325558 | | | CH.12_hs gi\|6056302 | 1.6 |
| 325656 | | | CH.14_hs gi\|6056305 | 1.6 |
| 326120 | | | CH.17_hs gi\|5867194 | 1.5 |
| 326139 | | | CH.17_hs gi\|5867203 | 1.5 |
| 326855 | | | CH.20_hs gi\|6552460 | 1.5 |
| 327438 | | | CH.02_hs gi\|6004454 | 1.6 |
| 329733 | | | CH.14_p2 gi\|6065783 | 1.6 |
| 330931 | F01443 | Hs.284256 | ESTs | 4.6 |
| 331591 | N71677 | Hs.42146 | ESTs | 1.9 |
| 332159 | AA621393 | Hs.112984 | EST | 1.5 |
| 332364 | W94688 | Hs.103253 | perilipin | 2.1 |
| 332502 | H21819 | Hs.14896 | Homo sapiens clone 24590 mRNA sequenc | 1.5 |
| 334175 | | | CH22_FGENES.349_10 | 1.5 |
| 334347 | | | CH22_FGENES.375_31 | 1.8 |
| 334737 | | | CH22_FGENES.424_12 | 1.8 |
| 335352 | | | CH22_FGENES.539_5 | 1.5 |
| 335639 | | | CH22_FGENES.584_19 | 1.6 |
| 336244 | | | CH22_FGENES.746_2 | 1.5 |
| 336336 | | | CH22_FGENES.814_8 | 1.7 |
| 336865 | | | CH22_FGENES.305-1 | 1.6 |
| 337494 | | | CH22_FGENES.799-12 | 1.6 |
| 337764 | | | CH22_EM: AC000097.GENSCAN.119-1 | 1.8 |
| 337983 | | | CH22_EM: AC005500.GENSCAN.110-1 | 2 |
| 338192 | | | CH22_EM: AC005500.GENSCAN.228-1 | 1.5 |
| 339366 | | | CH22_BA354I12.GENSCAN.34-2 | 1.5 |

Pkey: Unique Eos probeset identifier number
ExAccn: Exemplar Accession number, Genbank accession number
UnigeneID: Unigene number
Unigene Title: Unigene gene title
R1: Ratio of normal breast tissue to tumor

TABLE 15A
Table 15A shows the accession numbers
for those pkeys lacking unigeneID's for Table 15.
For each probeset, we have listed the gene
cluster number from which the oligonucleotides
were designed. Gene clusters were compiled
using sequences derived from Genbank ESTs
and mRNAs. These sequences were clustered
based on sequence similarity using Clustering
and Alignment Tools (DoubleTwist, Oakland
California). The Genbank accession numbers
for sequences comprising each cluster are
listed in the “Accession” column.

| Pkey | CAT number | Accession |
|---|---|---|
| 126300 | 250375_2 | D81972 BE003132 |
| 112538 | 504579_1 | AA908813 R70255 |
| 123505 | genbank_AA600135 | AA600135 |
| 104672 | 6735_7 | AA349096 AI368018 F21390 |
| | | F17759 R48772 AI421485 |
| | | AI300352 H43971 AI378525 |
| | | F33652 R47898 AI264177 |
| | | F22289 N28263 AI276281 |
| | | R48205 AI245302 AI190036 |
| | | AI281050 AW242903 H42892 |
| | | AA910870 AW473816 H25721 |
| | | AW451438 F19647 F22375 |
| | | H45809 F33447 AA774528 |
| | | AA007629 H42537 C01077 |
| | | F32386 |
| 322102 | 46708_1 | H45589 H19807 AF075038 H19808 H42437 |
| 336865 | CH22_4590FG_305_1_{—} | |
| 338192 | CH22_6755FG_LINK_EM: AC00 | |
| 329733 | c14_p2 | |
| 326120 | c17_hs | |
| 326139 | c17_hs | |
| 326855 | c20_hs | |
| 335352 | CH22_2699FG_539_5 LINK_EM | |
| 335639 | CH22_2999FG_584_19_LINK_E | |
| 307206 | AI192534 | |
| 307377 | AI222691 | |
| 337494 | CH22_5727FG_799_12_{—} | |
| 337764 | CH22_6115FG_LINK_EM: AC00 | |
| 337983 | CH22_6438FG_LINK_EM: AC00 | |
| 339366 | CH22_8336FG_LINK_BA354I1 | |
| 325272 | c11_hs | |
| 325558 | c12_hs | |
| 325656 | c14_hs | |
| 334175 | CH22_1455FG_349_10_LINK_E | |
| 304182 | H91086 | |
| 334347 | CH22_1640FG_375_31_LINK_E | |
| 327438 | c_2_hs | |
| 304622 | AA516384 | |
| 334737 | CH22_2049FG_424_12_LINK_E | |
| 304682 | AA550994 | |
| 336244 | CH22_3642FG_746_2_LINK_DA | |
| 306193 | AA923457 | |
| 336336 | CH22_3746FG_814_8_LINK_BA | |

Pkey: Unique Eos probeset identifier number
CAT number: Gene cluster number
Accession: Genbank accession numbers

TABLE 15B
Table 15B shows the genomic positioning
for those pkeys lacking unigene ID's and
accession numbers in Table 15. For each
predicted exon, we have listed the genomic
sequence source used for prediction.
Nucleotide locations of each predicted exon
are also listed.

| Pkey | Ref | Strand | Nt_position |
|---|---|---|---|
| 334347 | Dunham, I. et.al. | Plus | 13663814-13663926 |
| 334737 | Dunham, I. et.al. | Plus | 15998517-15998685 |
| 335639 | Dunham, I. et.al. | Plus | 25173591-25173696 |
| 337494 | Dunham, I. et.al. | Plus | 33339024-33339148 |
| 334175 | Dunham, I. et.al. | Minus | 11668659-11668597 |
| 335352 | Dunham, I. et.al. | Minus | 22681512-22681384 |
| 336244 | Dunham, I. et.al. | Minus | 31402729-31402583 |
| 336336 | Dunham, I. et.al. | Minus | 33797209-33797076 |
| 336865 | Dunham, I. et.al. | Minus | 8622405-8622289 |
| 337764 | Dunham, I. et.al. | Minus | 4035640-4035446 |
| 337983 | Dunham, I. et.al. | Minus | 7275495-7275271 |
| 338192 | Dunham, I. et.al. | Minus | 13248453-13248277 |
| 339366 | Dunham, I. et.al. | Minus | 33647431-33647293 |
| 325272 | 5866902 | Minus | 13247-13312 |
| 325558 | 6056302 | Plus | 70930-71030 |
| 325656 | 6056305 | Minus | 78190-78707 |
| 329733 | 6065783 | Plus | 163237-163450 |
| 326120 | 5867194 | Plus | 36116-36276 |
| 326139 | 5867203 | Minus | 218901-218960 |
| 326855 | 6552460 | Minus | 111390-111463 |
| 327438 | 6004454 | Minus | 199569-199692 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 16
Table 4 from BRCA 001-5 US
Table 16, a subset of table 15, depicts a preferred group of
genes highly downregulated in breast cancer cells.

| Pkey | ExAccn | UniGene ID | Unigene Title | R1 |
|---|---|---|---|---|
| 100502 | TIGR: HT1496 | Hs.169228 | Adrenal-Specific Protein Pg2 | 2.3 |
| 101367 | M12963 | Hs.4 | alcohol dehydrogenase 1 (class I); alpha | 2.9 |
| 102857 | X00129 | Hs.76461 | retinol-binding protein 4; interstitial | 3 |
| 104672 | AA007629 | | glycerol-3-phosphate dehydrogenase 1 | 2.4 |
| 107099 | AA609645 | Hs.211568 | eukaryotic translation initiation factor 4 gam | 2.7 |
| 108604 | AA099820 | Hs.49696 | ESTs | 2.4 |
| 115949 | AA443800 | Hs.43125 | ESTs | 2 |
| 115965 | AA446661 | Hs.173233 | ESTs | 2.2 |
| 119175 | R71792 | Hs.301002 | ESTs; Weakly similar to cell death activator | 2.8 |
| 119798 | W73386 | Hs.249129 | ESTs | 3 |
| 122127 | AA434447 | Hs.106771 | ESTs | 2.5 |
| 122348 | AA443695 | Hs.293410 | ESTs | 2.1 |
| 129285 | T62068 | Hs.11006 | ESTs | 2.1 |
| 131267 | AA211776 | Hs.2504 | myomesin 1 (skelemin) (185 kD) | 3.8 |
| 131282 | M12272 | Hs.4 | alcohol dehydrogenase 3 (class I); gamma | 2.2 |
| 131810 | D49487 | Hs.194236 | leptin (murine obesity homolog) | 2.5 |
| 133120 | X64559 | Hs.65424 | tetranectin (plasminogen-binding protein) | 2 |
| 133601 | S95936 | Hs.284176 | transferrin | 2.3 |
| 134111 | N79674 | Hs.8022 | TU3A protein | 4.6 |
| 301396 | AA923549 | Hs.224121 | ESTs | 2.1 |
| 311794 | AW238092 | Hs.254759 | ESTs | 2.1 |
| 312575 | H25237 | Hs.306814 | ESTs | 2.3 |
| 313283 | W32480 | Hs.157099 | ESTs | 2.2 |
| 322814 | AI824495 | Hs.211038 | ESTs | 2.2 |
| 322929 | AI365585 | Hs.146246 | ESTs | 2.3 |
| 324675 | AW014734 | Hs.157969 | ESTs | 2.2 |
| 330931 | F01443 | Hs.284256 | ESTs | 4.6 |
| 332364 | W94688 | Hs.103253 | perilipin | 2.1 |
| 337983 | | | CH22_EM: AC005500.GENSCAN.110-1 | 2 |

Pkey: Unique Eos probeset identifier number
ExAccn: Exemplar Accession number, Genbank accession number
UnigeneID: Unigene number
Unigene Title: Unigene gene title
R1: Ratio of normal breast tissue to tumor

TABLE 16A
Table 16A shows the accession numbers for
those pkeys lacking unigeneID's for Table 16.
For each probeset, we have listed the gene
cluster number from which the oligonucleotides
were designed. Gene clusters were compiled
using sequences derived from Genbank ESTs
and mRNAs. These sequences were clustered
based on sequence similarity using Clustering
and Alignment Tools (DoubleTwist, Oakland
California). The Genbank accession numbers
for sequences comprising each cluster are
listed in the “Accession” column.

| Pkey | CAT number | Accession |
|---|---|---|
| 104672 | 6735_7 | AA349096 AI368018 F21390 F17759 |
| | | R48772 AI421485 AI300352 H43971 |
| | | AI378525 F33652 R47898 AI264177 |
| | | F22289 N28263 AI276281 R48205 |
| | | AI245302 AI190036 AI281050 AW242903 |
| | | H42892 AA910870 AW473816 H25721 |
| | | AW451438 F19647 F22375 H45809 |
| | | F33447 AA774528 AA007629 |
| | | H42537 C01077 F32386 |

Pkey: Unique Eos probeset identifier number
CAT number: Gene cluster number
Accession: Genbank accession numbers

TABLE 17
Table 1 from BRCA 014 P

| Pkey | ExAccn | UnigeneID | Unigene Title | R1 |
|---|---|---|---|---|
| 100227 | AV654694 | Hs.82316 | interferon-induced, hepatitis C-associat | 3 |
| 100405 | AW291587 | Hs.82733 | nidogen 2 | 3.2 |
| 100406 | AI962060 | Hs.118397 | AE-binding protein 1 | 3.6 |
| 100420 | D86983 | Hs.118893 | Melanoma associated gene | 3.2 |
| 100911 | X83300 | Hs.289103 | SMA4 | 5.2 |
| 100960 | J00124 | Hs.117729 | keratin 14 (epidermolysis bullosa simple | 4.3 |
| 101011 | BE387036 | Hs.1211 | acid phosphatase 5, tartrate resistant | 3 |
| 101183 | AA442324 | Hs.795 | H2A histone family, member O | 3.2 |
| 101194 | L20971 | Hs.188 | phosphodiesterase 4B, cAMP-specific (dun | 3 |
| 101329 | U66042 | Hs.82171 | Homo sapiens clone 191B7 placenta expres | 4.1 |
| 101378 | BE563085 | Hs.833 | interferon-stimulated protein, 15 kDa | 5.3 |
| 101474 | R07566 | Hs.73817 | small inducible cytokine A3 (homologous | 3.9 |
| 101491 | M25809 | Hs.64173 | ATPase, H+ transporting, lysosomal (vacu | 4.5 |
| 101530 | M29874 | Hs.1360 | cytochrome P450, subfamily IIB (phenobar | 9 |
| 101602 | AA353776 | Hs.901 | CD48 antigen (B-cell membrane protein) | 3.4 |
| 101663 | NM_003528 | Hs.2178 | H2B histone family, member Q | 5.6 |
| 101758 | BE019494 | Hs.79217 | pyrroline-5-carboxylate reductase 1 | 3.6 |
| 101767 | M81057 | Hs.180884 | carboxypeptidase B1 (tissue) | 12 |
| 101817 | M89907 | Hs.152292 | SWI/SNF related, matrix associated, acti | 3.2 |
| 101851 | BE260964 | Hs.82045 | midkine (neurite growth-promoting factor | 4.1 |
| 101878 | M97815 | Hs.183650 | cellular retinoic acid-binding protein 2 | 6.5 |
| 102209 | NM_002038 | Hs.265827 | interferon, alpha-inducible protein (clo | 3 |
| 102214 | U23752 | Hs.32964 | SRY (sex determining region Y)-box 11 | 3 |
| 102297 | NM_001504 | Hs.198252 | G protein-coupled receptor 9 | 3.7 |
| 102299 | NM_005824 | Hs.155545 | 37 kDa leucine-rich repeat (LRR) protein | 3.7 |
| 102301 | NM_005651 | Hs.183671 | tryptophan 2,3-dioxygenase | 5.2 |
| 102305 | AL043202 | Hs.90073 | chromosome segregation 1 (yeast homolog) | 3.5 |
| 102369 | U39840 | Hs.299867 | hepatocyte nuclear factor 3, alpha | 3.9 |
| 102591 | U62325 | Hs.324125 | amyloid beta (A4) precursor protein-bind | 4 |
| 102721 | H16646 | Hs.118666 | hypothetical protein PP591 | 3.5 |
| 102739 | AA363025 | Hs.155572 | Human clone 23801 mRNA sequence | 3.2 |
| 102791 | AF080229 | | gb: Human endogenous retrovirus K clone 1 | 3 |
| 102804 | NM_002318 | Hs.83354 | lysyl oxidase-like 2 | 3.2 |
| 102903 | M73779 | Hs.250505 | retinoic acid receptor, alpha | 3.3 |
| 103010 | X52509 | Hs.161640 | tyrosine aminotransferase | 12.4 |
| 103042 | T81656 | Hs.252259 | ribosomal protein S3 | 4.5 |
| 103117 | X63578 | Hs.295449 | parvalbumin | 3 |
| 103207 | X72790 | | gb: Human endogenous retrovirus mRNA for | 5.9 |
| 103282 | BE390551 | Hs.77628 | steroidogenic acute regulatory protein r | 3.9 |
| 103284 | AI751601 | Hs.8375 | TNF receptor-associated factor 4 | 3.3 |
| 103329 | X85134 | Hs.72984 | retinoblastoma-binding protein 5 | 3.1 |
| 103364 | X90872 | Hs.279929 | gp25L2 protein | 3 |
| 103385 | NM_007069 | Hs.37189 | similar to rat HREV107 | 3.4 |
| 103456 | AA496425 | Hs.9629 | papillary renal cell carcinoma (transloc | 3.2 |
| 103498 | Y09306 | Hs.30148 | homeodomain-interacting protein kinase 3 | 3.4 |
| 103558 | BE616547 | Hs.2785 | keratin 17 | 3.7 |
| 103563 | L02911 | Hs.150402 | Activin A receptor, type I (ACVR1) (ALK | 3.2 |
| 103612 | BE336654 | Hs.70937 | H3 histone family, member A | 4.5 |
| 103825 | AI571835 | Hs.55468 | ESTs | 4 |
| 104073 | AW779318 | Hs.88417 | ESTs | 3.8 |
| 104103 | AW021102 | Hs.21509 | ESTs | 4.3 |
| 104115 | AF183810 | Hs.26102 | opposite strand to trichorhinophalangeal | 7.6 |
| 104168 | AA461618 | Hs.31704 | ESTs, Weakly similar to KIAA0227 [H. sapi | 3.6 |
| 104173 | AA084273 | Hs.76561 | ESTs, Weakly similar to S47072 finger pr | 4 |
| 104181 | AF173296 | Hs.283740 | DC6 protein | 3 |
| 104189 | AB040927 | Hs.301804 | KIAA1494 protein | 3.2 |
| 104269 | AI559444 | Hs.293960 | ESTs | 4.3 |
| 104307 | AI929700 | Hs.111680 | endosulfine alpha | 3.1 |
| 104518 | H20816 | Hs.112423 | Homo sapiens mRNA; cDNA DKFZp586I1420 (f | 3.2 |
| 104556 | AV650851 | Hs.96900 | hypothetical protein; KIAA1830 protein | 4.4 |
| 104658 | AA360954 | Hs.27268 | Homo sapiens cDNA: FLJ21933 fis, clone H | 3.2 |
| 104748 | AA015879 | Hs.33536 | ESTs | 3.2 |
| 104755 | T49951 | Hs.9029 | DKFZP434G032 protein | 4.5 |
| 104825 | AA035613 | Hs.141883 | ESTs | 6.9 |
| 104830 | AW294092 | Hs.21594 | hypothetical protein MGC15754 | 11.1 |
| 104865 | T79340 | Hs.22575 | B-cell CLL/lymphoma 6, member B (zinc fi | 3.5 |
| 104906 | BE298684 | Hs.26802 | protein kinase domains containing protei | 6.5 |
| 104961 | H78517 | Hs.33905 | ESTs | 3.6 |
| 105038 | AW503733 | Hs.9414 | KIAA1488 protein | 4.5 |
| 105088 | H58589 | Hs.35156 | Homo sapiens cDNA FLJ11027 fis, clone PL | 3.8 |
| 105092 | AA148982 | Hs.29068 | ESTs | 3 |
| 105093 | AL137566 | Hs.32405 | Homo sapiens mRNA; cDNA DKFZp586G0321 (f | 4.8 |
| 105304 | AW134924 | Hs.190325 | ESTs | 8.2 |
| 105397 | AA814807 | Hs.7395 | hypothetical protein FLJ23182 | 3.1 |
| 105409 | AW505076 | Hs.301855 | DiGeorge syndrome critical region gene 8 | 4.2 |
| 105431 | AA252033 | Hs.242413 | hypothetical protein DKFZp434K1421 | 4.4 |
| 105552 | AA256750 | Hs.28802 | centaurin-alpha 2 protein | 3.2 |
| 105598 | AA279439 | Hs.279763 | hypothetical protein FLJ10504 | 3.5 |
| 105650 | W16741 | Hs.25635 | HSPC003 protein | 3.7 |
| 105688 | AI299139 | Hs.17517 | ESTs | 5.5 |
| 105808 | AI133161 | Hs.286131 | CGI-101 protein | 3.5 |
| 105809 | AW973653 | Hs.20104 | hypothetical protein FLJ00052 | 3.3 |
| 105909 | AA195191 | Hs.5111 | hypothetical protein FLJ20729 | 3.2 |
| 105965 | AA131657 | Hs.23830 | ESTs | 3.3 |
| 106135 | AL117474 | Hs.41181 | Homo sapiens mRNA; cDNA DKFZp727C191 (fr | 3.2 |
| 106184 | W28948 | Hs.10762 | ESTs | 3.3 |
| 106293 | N39842 | Hs.301444 | KIAA1673 | 4.1 |
| 106400 | BE397649 | Hs.94109 | Homo sapiens cDNA FLJ13634 fis, clone PL | 3.1 |
| 106474 | BE383668 | Hs.42484 | hypothetical protein FLJ10618 | 3.2 |
| 106484 | AA351978 | Hs.4943 | hepatocellular carcinoma associated prot | 7.8 |
| 106533 | AL134708 | Hs.145998 | ESTs | 3 |
| 106614 | AA648459 | Hs.335951 | hypothetical protein AF301222 | 3.8 |
| 106636 | AW958037 | Hs.286 | ribosomal protein L4 | 3.3 |
| 106661 | AW499914 | Hs.7579 | hypothetical protein FLJ10402 | 3 |
| 106743 | BE613328 | Hs.21938 | hypothetical protein FLJ12492 | 4.2 |
| 106844 | AA485055 | Hs.158213 | sperm associated antigen 6 | 3.4 |
| 106864 | AI311928 | | gb: qo89h04.x1 NCI_CGAP_Kid5 Homo sapiens | 4.4 |
| 106865 | AW192535 | Hs.19479 | ESTs | 3.6 |
| 106871 | AW472981 | Hs.321130 | hypothetical protein MGC2771 | 4.1 |
| 106942 | AA995351 | Hs.31314 | retinoblastoma-binding protein 7 | 3.6 |
| 106968 | AF216751 | Hs.26813 | CDA14 | 5.3 |
| 107105 | AW963419 | Hs.155223 | stanniocalcin 2 | 3.4 |
| 107158 | N32849 | Hs.31844 | hypothetical protein FLJ12586 | 3.1 |
| 107248 | AW263124 | Hs.315111 | nuclear receptor co-repressor/HDAC3 comp | 5.9 |
| 107265 | BE379594 | Hs.49136 | ESTs, Moderately similar to ALU7_HUMAN A | 3.9 |
| 107630 | AW961576 | Hs.60178 | ESTs | 4.6 |
| 107710 | AI955040 | Hs.265398 | ESTs, Weakly similar to transformation-r | 3 |
| 107890 | AA025386 | Hs.61311 | ESTs, Weakly similar to S10590 cysteine | 3.1 |
| 107985 | T40064 | Hs.71968 | Homo sapiens mRNA; cDNA DKFZp564F053 (fr | 4.8 |
| 108000 | AI263307 | Hs.239884 | H2B histone family, member L | 3.3 |
| 108217 | AA058686 | Hs.62588 | ESTs | 3.8 |
| 108435 | T82427 | Hs.194101 | Homo sapiens cDNA: FLJ20869 fis, clone A | 3 |
| 108591 | AB033073 | Hs.43857 | similar to glucosamine-6-sulfatases | 3.3 |
| 108733 | AA121022 | | gb: zn84f10.r1 Stratagene lung carcinoma | 3.9 |
| 108771 | AF068290 | Hs.79741 | hypothetical protein FLJ10116 | 6.1 |
| 108819 | AA011449 | Hs.271627 | ESTs | 3.6 |
| 108912 | AA136674 | Hs.118681 | EST | 3.9 |
| 109086 | AF186114 | Hs.270737 | tumor necrosis factor (ligand) superfami | 3.7 |
| 109124 | AK000684 | Hs.183887 | hypothetical protein FLJ22104 | 3.1 |
| 109132 | AI970536 | Hs.16603 | hypothetical protein FLJ13163 | 3.7 |
| 109163 | N23235 | Hs.30567 | ESTs, Weakly similar to B34087 hypotheti | 4.5 |
| 109277 | AA196443 | Hs.86043 | Homo sapiens cDNA FLJ13558 fis, clone PL | 3.7 |
| 109410 | AW504732 | Hs.21275 | hypothetical protein FLJ11011 | 4.6 |
| 109454 | AA232255 | Hs.295232 | ESTs, Moderately similar to A46010 X-lin | 6.4 |
| 109514 | AA234087 | Hs.262346 | ESTs, Weakly similar to S72482 hypotheti | 4.8 |
| 109581 | R45584 | Hs.23025 | ESTs, Weakly similar to ALU5_HUMAN ALU S | 3.3 |
| 109632 | AA325138 | Hs.235873 | hypothetical protein FLJ22672 | 3 |
| 109644 | AW973964 | Hs.291531 | ESTs, Highly similar to 1203217A dehydro | 3 |
| 109700 | F09609 | | gb: HSC33H092 normalized infant brain cDN | 3.2 |
| 109768 | F06838 | Hs.14763 | ESTs | 3.2 |
| 109807 | R43646 | Hs.12422 | ESTs | 3.8 |
| 109842 | AW818436 | Hs.23590 | solute carrier family 16 (monocarboxylic | 3.3 |
| 109895 | AK001680 | Hs.30488 | DKFZP434F091 protein | 3.6 |
| 110024 | AW973152 | Hs.31050 | ESTs | 4.2 |
| 110561 | AA379597 | Hs.5199 | HSPC150 protein similar to ubiquitin-con | 5.1 |
| 110675 | H89355 | Hs.249159 | adrenergic, alpha-2A-, receptor | 5.3 |
| 110707 | AI239832 | Hs.15617 | ESTs, Weakly similar to ALU4_HUMAN ALU S | 3.7 |
| 110915 | BE092285 | Hs.29724 | hypothetical protein FLJ13187 | 3.7 |
| 111139 | N64683 | Hs.290943 | ESTs | 4 |
| 111155 | N66563 | Hs.191358 | ESTs | 3.1 |
| 111199 | AI767435 | Hs.29822 | ESTs | 4.5 |
| 111336 | AI457338 | Hs.29894 | ESTs | 5.4 |
| 111510 | R07856 | Hs.16355 | ESTs | 3.2 |
| 111532 | R08440 | | gb: yf19f09.s1 Soares fetal liver spleen | 3.1 |
| 111689 | AA602004 | Hs.23260 | ESTs | 3.2 |
| 111823 | R35252 | Hs.24944 | ESTs, Weakly similar to 2109260A B cell | 3.3 |
| 111876 | R38239 | Hs.293246 | ESTs, Weakly similar to putative p150 [H | 3.1 |
| 111892 | AA421081 | Hs.12388 | ESTs | 3.4 |
| 111893 | AF070526 | Hs.13429 | Homo sapiens clone 24787 mRNA sequence | 3.3 |
| 112125 | AW379029 | Hs.118338 | ESTs, Weakly similar to unnamed protein | 4.4 |
| 112170 | BE246743 | Hs.288529 | hypothetical protein FLJ22635 | 7.3 |
| 112287 | AB033064 | Hs.334806 | KIAA1238 protein | 3.2 |
| 112300 | H24334 | Hs.26125 | ESTs | 4.4 |
| 112303 | R54797 | | gb: yg87b07.s1 Soares infant brain 1NIB H | 3.4 |
| 112478 | R66067 | Hs.28664 | ESTs | 8.2 |
| 112561 | AI791493 | Hs.129873 | ESTs, Weakly similar to A36036 cytochrom | 5.5 |
| 112631 | R82040 | | gb: yj06b06.s1 Soares placenta Nb2HP Homo | 3.9 |
| 112637 | R82331 | Hs.164599 | ESTs | 5.4 |
| 112657 | AW844878 | Hs.19769 | hypothetical protein MGC4174 | 3.2 |
| 112678 | AI418466 | Hs.33665 | ESTs | 4.7 |
| 112917 | AA082465 | Hs.125031 | choline/ethanolaminephosphotransferase | 3.7 |
| 113070 | AB032977 | Hs.6298 | KIAA1151 protein | 3.1 |
| 113095 | AA828380 | Hs.126733 | ESTs | 3.4 |
| 113117 | AW813731 | Hs.159153 | ESTs, Moderately similar to S65657 alpha | 3.4 |
| 113187 | BE613410 | Hs.31575 | SEC63, endoplasmic reticulum translocon | 3.2 |
| 113200 | T57773 | Hs.10263 | ESTs | 3.5 |
| 113206 | BE262470 | Hs.241471 | RNB6 | 6.2 |
| 113374 | T79925 | Hs.269165 | ESTs, Weakly similar to ALU1_HUMAN ALU S | 3.7 |
| 113440 | U54727 | Hs.191445 | ESTs | 3 |
| 113494 | T91451 | Hs.86538 | ESTs | 3.4 |
| 113518 | AW367788 | Hs.323954 | postmeiotic segregation increased 2-like | 3.1 |
| 113571 | AI702609 | Hs.15713 | hypothetical protein MGC2776 | 3.1 |
| 113822 | NM_004585 | Hs.17466 | retinoic acid receptor responder (tazaro | 3.9 |
| 113835 | AI912410 | Hs.27475 | Homo sapiens cDNA FLJ12749 fis, clone NT | 3 |
| 113938 | W81598 | | gb: zd88g02.s1 Soares_fetal_heart_NbHH19W | 4.6 |
| 113947 | W84768 | | gb: zh53d03.s1 Soares_fetal_liver_spleen_{—} | 3.1 |
| 113970 | W27249 | Hs.8109 | hypothetical protein FLJ21080 | 6.9 |
| 114086 | AA378776 | Hs.288649 | hypothetical protein MGC3077 | 4.3 |
| 114148 | AW470411 | Hs.288433 | neurotrimin | 4.1 |
| 114424 | AW780192 | Hs.267596 | ESTs | 3.4 |
| 114518 | AW163267 | Hs.106469 | suppressor of var1 (S. cerevisiae) 3-like | 3.1 |
| 114563 | AI979168 | Hs.82226 | glycoprotein (transmembrane) nmb | 4.8 |
| 114965 | AI733881 | Hs.72472 | BMP-R1B | 10.1 |
| 114995 | AA769266 | Hs.193657 | ESTs | 3.6 |
| 115121 | AI634549 | Hs.88155 | ESTs | 3.2 |
| 115134 | AW968073 | Hs.194331 | ESTs, Highly similar to A55713 inositol | 4.2 |
| 115167 | AA749209 | Hs.43728 | hypothetical protein | 3 |
| 115253 | BE149845 | Hs.289038 | hypothetical protein MGC4126 | 3.6 |
| 115277 | AA814100 | Hs.86693 | ESTs | 3.9 |
| 115327 | N46436 | Hs.109221 | ESTs | 3.4 |
| 115354 | AA281636 | Hs.334827 | ESTs | 4.8 |
| 115657 | AA405620 | Hs.55158 | ESTs, Weakly similar to T29520 hypotheti | 3.5 |
| 115676 | AA953006 | Hs.88143 | ESTs | 9.3 |
| 115709 | AW293849 | Hs.58279 | ESTs, Weakly similar to ALU7_HUMAN ALU S | 3.4 |
| 115729 | AA417812 | Hs.38775 | ESTs | 4 |
| 115787 | AI126772 | Hs.40479 | ESTs | 3.1 |
| 115830 | AW970529 | Hs.86434 | hypothetical protein FLJ21816 | 3.6 |
| 115835 | AA521410 | Hs.41371 | ESTs | 3.1 |
| 115850 | NM_014937 | Hs.52463 | KIAA0966 protein | 3 |
| 115900 | AK001500 | Hs.165186 | hypothetical protein FLJ13852 | 3.2 |
| 115935 | AA354549 | Hs.41181 | Homo sapiens mRNA; cDNA DKFZp727C191 (fr | 3 |
| 115948 | AL042465 | Hs.43445 | poly(A)-specific ribonuclease (deadenyla | 3.1 |
| 116092 | AB041035 | Hs.93847 | NM_016931: Homo sapiens NADPH oxidase 4 ( | 6.7 |
| 116115 | AL042355 | Hs.70202 | WD repeat domain 10 | 3.6 |
| 116184 | AW450737 | Hs.128791 | CGI-09 protein | 3.1 |
| 116192 | AA464976 | Hs.62528 | ESTs, Moderately similar to A46010 X-lin | 3.3 |
| 116208 | AI219083 | Hs.42532 | ESTs, Moderately similar to ALU8_HUMAN A | 3.2 |
| 116246 | AF265555 | Hs.250646 | baculoviral IAP repeat-containing 6 | 3.6 |
| 116443 | AW962196 | Hs.321264 | LBP protein 32 | 4.1 |
| 116470 | AI272141 | Hs.83484 | SRY (sex determining region Y)-box 4 | 4.1 |
| 116726 | AK001114 | Hs.53913 | hypothetical protein FLJ10252 | 8.6 |
| 116845 | AA649530 | | gb: ns44f05.s1 NCI_CGAP_Alv1 Homo sapiens | 3.2 |
| 117026 | H88256 | Hs.50456 | ESTs, Moderately similar to ZN75_HUMAN Z | 3.5 |
| 117216 | AI569804 | Hs.42792 | ESTs, Weakly similar to I78885 serine/th | 3.1 |
| 117296 | AL133427 | Hs.42506 | Homo sapiens mRNA full length insert cDN | 3.2 |
| 117403 | H84455 | Hs.40639 | ESTs | 4.7 |
| 117691 | AB040959 | Hs.93836 | DKFZP434N014 protein | 3 |
| 118229 | AW968941 | Hs.166254 | hypothetical protein DKFZp566I133 | 3.3 |
| 118363 | AI183838 | Hs.48938 | hypothetical protein FLJ21802 | 4.3 |
| 118416 | N66028 | Hs.49105 | FKBP-associated protein | 3.1 |
| 118470 | AW970584 | Hs.291033 | ESTs | 3.4 |
| 118502 | AL157488 | Hs.50150 | Homo sapiens mRNA; cDNA DKFZp564B182 (fr | 5.2 |
| 118695 | AK000465 | Hs.50081 | KIAA1199 protein | 3.4 |
| 118925 | N92293 | Hs.206832 | ESTs, Moderately similar to ALU8_HUMAN A | 3.3 |
| 119025 | BE003760 | Hs.55209 | Homo sapiens mRNA; cDNA DKFZp434K0514 (f | 19.7 |
| 119036 | R95872 | Hs.117572 | chemokine binding protein 2 | 3.7 |
| 119063 | R16833 | Hs.53106 | ESTs, Moderately similar to ALU1_HUMAN A | 4.1 |
| 119075 | M10905 | Hs.287820 | fibronectin 1 | 3.2 |
| 119620 | W47620 | Hs.56009 | 2′-5′-oligoadenylate synthetase 3 (100 k | 3.3 |
| 119741 | AF041853 | Hs.43670 | kinesin family member 3A | 3.1 |
| 119747 | AI970797 | Hs.64859 | ESTs | 5 |
| 119754 | AL037824 | Hs.194695 | ras homolog gene family, member I | 3.8 |
| 119905 | AW449064 | Hs.119571 | collagen, type III, alpha 1 (Ehlers-Danl | 3.1 |
| 120084 | W94472 | Hs.59529 | ESTs, Moderately similar to ALU1_HUMAN A | 8.4 |
| 120241 | AA825686 | Hs.321176 | ESTs, Weakly similar to S65824 reverse t | 3.6 |
| 120326 | AA196300 | Hs.21145 | hypothetical protein RG083M05.2 | 3.2 |
| 120742 | AA225084 | | gb: nc21d06.r1 NCI_CGAP_Pr1 Homo sapiens | 3.6 |
| 120870 | AA357172 | Hs.292581 | ESTs, Moderately similar to ALU1_HUMAN A | 5.8 |
| 120885 | AA365515 | Hs.301872 | hypothetical protein MGC4840 | 3 |
| 120970 | AA398118 | Hs.97579 | ESTs, Weakly similar to A46010 X-linked | 3.7 |
| 121054 | AW976570 | Hs.97387 | ESTs | 5.3 |
| 121095 | AA320134 | Hs.196029 | Homo sapiens mRNA for KIAA1657 protein, | 4 |
| 121103 | AA398936 | Hs.97697 | EST | 3.5 |
| 121121 | AA399371 | Hs.189095 | similar to SALL1 (sal (Drosophila)-like | 6.3 |
| 121337 | AW885727 | Hs.301570 | ESTs | 4.7 |
| 121351 | AW206227 | Hs.287727 | hypothetical protein FLJ23132 | 5 |
| 121611 | M31669 | Hs.1735 | inhibin, beta B (activin AB beta polypep | 3.6 |
| 121643 | AA640987 | Hs.193767 | ESTs | 5.6 |
| 121770 | NM_015902 | Hs.278428 | progestin induced protein | 3.4 |
| 122125 | AK000492 | Hs.98806 | hypothetical protein | 4.1 |
| 122338 | AA443311 | Hs.98998 | ESTs | 3 |
| 122417 | AA446965 | Hs.112092 | ESTs | 4.7 |
| 122513 | AI767879 | Hs.99214 | ESTs | 3.8 |
| 122544 | AW973253 | Hs.292689 | ESTs | 3 |
| 122655 | AA323296 | Hs.97837 | Homo sapiens mRNA; cDNA DKFZp547J047 (fr | 5.6 |
| 122805 | AA526911 | Hs.82772 | collagen, type XI, alpha 1 | 3.2 |
| 122851 | AW205931 | Hs.99598 | hypothetical protein MGC5338 | 8.6 |
| 123105 | AA487809 | Hs.166011 | catenin (cadherin-associated protein), d | 3 |
| 123111 | AA228776 | Hs.191721 | ESTs | 6.9 |
| 123249 | AA371307 | Hs.125056 | ESTs | 3.6 |
| 123273 | AA491253 | Hs.173611 | Empirically selected from AFFX single pr | 7 |
| 123385 | BE149685 | Hs.17767 | KIAA1554 protein | 3.1 |
| 123419 | T66087 | Hs.112482 | Homo sapiens unknown mRNA sequence | 3.4 |
| 123485 | AI308876 | Hs.103849 | hypothetical protein DKFZp761D112 | 3.1 |
| 123645 | AI675944 | Hs.188691 | Homo sapiens cDNA FLJ12033 fis, clone HE | 3.8 |
| 123819 | AA580082 | Hs.112264 | ESTs | 4.7 |
| 124012 | AA352723 | Hs.241471 | RNB6 | 3.8 |
| 124243 | H69125 | Hs.133525 | ESTs | 4.1 |
| 124357 | N22401 | | gb: yw37g07.s1 Morton Fetal Cochlea Homo | 4.1 |
| 124359 | N22508 | Hs.139315 | Homo sapiens cDNA: FLJ21479 fis, clone C | 3.6 |
| 124567 | AW451645 | Hs.151504 | Homo sapiens cDNA FLJ11973 fis, clone HE | 3.1 |
| 124911 | N34151 | Hs.174195 | interferon induced transmembrane protein | 3.5 |
| 124972 | R41396 | Hs.101774 | hypothetical protein FLJ23045 | 4.3 |
| 125006 | BE065136 | Hs.145696 | splicing factor (CC1.3) | 6 |
| 125042 | T78906 | Hs.269432 | ESTs, Moderately similar to ALU1_HUMAN A | 8.1 |
| 125184 | W60326 | Hs.288684 | Homo sapiens cDNA FLJ11750 fis, clone HE | 4.7 |
| 125243 | AW970536 | Hs.105413 | ESTs | 3.1 |
| 125286 | AF086534 | Hs.187561 | ESTs, Moderately similar to ALU1_HUMAN A | 3.3 |
| 125304 | AL359573 | Hs.124940 | GTP-binding protein | 3 |
| 125330 | AW880562 | Hs.114574 | ESTs | 3 |
| 125331 | AI422996 | Hs.161378 | ESTs | 3.2 |
| 125685 | AI924630 | Hs.4943 | hepatocellular carcinoma associated prot | 3.2 |
| 126257 | N99638 | | gb: za39g11.r1 Soares fetal liver spleen | 4 |
| 126474 | AW975814 | Hs.326714 | Homo sapiens clone IMAGE: 713177, mRNA se | 4 |
| 126666 | AA648886 | Hs.151999 | ESTs | 3.8 |
| 126872 | AW450979 | | gb: UI-H-B13-ala-a-12-0-UI.s1 NCI_CGAP_Su | 3 |
| 127431 | AW771958 | Hs.175437 | ESTs, Moderately similar to PC4259 ferri | 3.6 |
| 127980 | AA961459 | Hs.125644 | ESTs | 4.1 |
| 127997 | AW068311 | Hs.311054 | Homo sapiens mRNA full length insert cDN | 3.3 |
| 128420 | AA650274 | Hs.41296 | fibronectin leucine rich transmembrane p | 4.6 |
| 128609 | NM_003616 | Hs.102456 | survival of motor neuron protein interac | 3.9 |
| 128946 | Y13153 | Hs.107318 | kynurenine 3-monooxygenase (kynurenine 3 | 3.1 |
| 128955 | AA775076 | Hs.185807 | Homo sapiens , Similar to PRO0478 protein | 3.9 |
| 129092 | D56365 | Hs.63525 | poly(rC)-binding protein 2 | 3.3 |
| 129270 | AA357185 | Hs.109918 | ras homolog gene family, member H | 3.1 |
| 129301 | AF182277 | Hs.330780 | cytochrome P450, subfamily IIB (phenobar | 3.9 |
| 129385 | AA172106 | Hs.110950 | Rag C protein | 6.2 |
| 129619 | AA209534 | Hs.284243 | tetraspan NET-6 protein | 3.4 |
| 129629 | AK000398 | Hs.11747 | hypothetical protein FLJ20391 | 3 |
| 129725 | X56411 | Hs.1219 | alcohol dehydrogenase 4 (class II), pi p | 3.2 |
| 130069 | AI754813 | Hs.146428 | collagen, type V, alpha 1 | 5.4 |
| 130092 | X03363 | Hs.323910 | v-erb-b2 avian erythroblastic leukemia v | 4.4 |
| 130298 | AI347487 | Hs.132781 | class I cytokine receptor | 4.6 |
| 130382 | NM_003450 | Hs.155204 | zinc finger protein 174 | 5.6 |
| 130622 | AI582291 | Hs.16846 | ESTs, Weakly similar to O4HUD1 debrisoqu | 3 |
| 130703 | R77776 | Hs.18103 | ESTs | 3.8 |
| 130881 | AA809875 | Hs.25933 | ESTs | 4.2 |
| 130954 | AB014544 | Hs.21572 | KIAA0644 gene product | 4.7 |
| 131095 | AI399653 | Hs.22917 | ESTs | 4.3 |
| 131153 | H09048 | Hs.23606 | ESTs | 3.8 |
| 131253 | R71802 | Hs.24853 | ESTs | 3.5 |
| 131372 | AW293399 | Hs.144904 | nuclear receptor co-repressor 1 | 3.6 |
| 131507 | AI826268 | Hs.27769 | ESTs, Weakly similar to MCAT_HUMAN MITOC | 3.2 |
| 131587 | AI695549 | Hs.183868 | glucuronidase, beta | 3.1 |
| 131739 | AF017986 | Hs.31386 | secreted frizzled-related protein 2 | 3.2 |
| 131795 | BE501849 | Hs.32317 | high-mobility group 20B | 3.2 |
| 131970 | D86960 | Hs.3610 | KIAA0205 gene product | 3.6 |
| 131986 | NM_002314 | Hs.36566 | LIM domain kinase 1 | 3.2 |
| 132093 | AA400091 | Hs.39421 | ESTs | 3.2 |
| 132122 | AA426202 | Hs.40403 | Cbp/p300-interacting transactivator, wit | 3 |
| 132159 | D76435 | Hs.41154 | Zic family member 1 (odd-paired Drosophi | 3.2 |
| 132333 | AA192669 | Hs.45032 | ESTs | 3.5 |
| 132406 | AL133731 | Hs.4774 | Homo sapiens mRNA; cDNA DKFZp761C1712 (f | 9.2 |
| 132482 | AV660345 | Hs.238126 | CGI-49 protein | 8.2 |
| 132543 | BE568452 | Hs.5101 | protein regulator of cytokinesis 1 | 3.4 |
| 132624 | AA326108 | Hs.33829 | bHLH protein DEC2 | 3.2 |
| 132700 | AA319233 | Hs.5521 | ESTs | 4.8 |
| 132725 | NM_006276 | Hs.184167 | splicing factor, arginine/serine-rich 7 | 3.6 |
| 132799 | W73311 | Hs.169407 | SAC2 (suppressor of actin mutations 2, y | 3.2 |
| 132847 | T48195 | Hs.58189 | eukaryotic translation initiation factor | 3.5 |
| 132857 | Y00272 | Hs.184572 | cell division cycle 2, G1 to S and G2 to | 4.4 |
| 132936 | AL120659 | Hs.6111 | aryl-hydrocarbon receptor nuclear transl | 4.8 |
| 133130 | AI128606 | Hs.6557 | zinc finger protein 161 | 3.3 |
| 133142 | AW952412 | Hs.65874 | ESTs, Weakly similar to A40348 Elav/Sex- | 3.5 |
| 133167 | AW162840 | Hs.6641 | kinesin family member 5C | 4.5 |
| 133225 | AW600291 | Hs.6823 | hypothetical protein FLJ10430 | 3.3 |
| 133274 | AA085191 | Hs.6949 | hypothetical protein MGC11275 | 3 |
| 133275 | Z93241 | Hs.239934 | CGI-96 protein | 4.5 |
| 133287 | AW797437 | Hs.69771 | B-factor, properdin | 4.1 |
| 133376 | BE618768 | Hs.7232 | acetyl-Coenzyme A carboxylase alpha | 5.1 |
| 133462 | AW675064 | Hs.73875 | fumarylacetoacetate hydrolase (fumarylac | 3 |
| 133740 | AW162919 | Hs.170160 | RAB2, member RAS oncogene family-like | 3.4 |
| 133831 | BE274552 | Hs.76578 | protein inhibitor of activated STAT3 | 3.9 |
| 133976 | AI908165 | Hs.169946 | GATA-binding protein 3 (T-cell receptor | 6.2 |
| 134666 | BE391929 | Hs.8752 | transmembrane protein 4 | 3.1 |
| 134710 | AI433797 | Hs.8889 | serine hydroxymethyltransferase 1 (solub | 3 |
| 134731 | D89377 | Hs.89404 | msh (Drosophila) homeo box homolog 2 | 5.8 |
| 134776 | J05582 | Hs.89603 | mucin 1, transmembrane | 4 |
| 135230 | AF064804 | Hs.96757 | suppressor of Ty (S. cerevisiae) 3 homolo | 3.2 |
| 135303 | R61253 | Hs.98265 | KIAA1877 protein | 3.3 |
| 135400 | X78592 | Hs.99915 | androgen receptor (dihydrotestosterone r | 4.8 |
| 135411 | L10333 | Hs.99947 | reticulon 1 | 3.8 |
| 300089 | AI199738 | Hs.208275 | ESTs, Weakly similar to ALUA_HUMAN !!!! | 3.8 |
| 300233 | AW614220 | Hs.189402 | ESTs | 4.2 |
| 300254 | AW183618 | Hs.55610 | solute carrier family 30 (zinc transport | 9.9 |
| 300256 | AW591433 | Hs.298241 | Transmembrane protease, serine 3 | 4.9 |
| 300378 | Z45270 | Hs.235873 | hypothetical protein FLJ22672 | 3.4 |
| 300973 | AA572949 | Hs.207566 | ESTs | 3.5 |
| 301111 | R10799 | Hs.191990 | ESTs | 3.8 |
| 301341 | AA887801 | Hs.208229 | G protein-coupled receptor | 13.9 |
| 301548 | AI091631 | Hs.203845 | two pore potassium channel KT3.3 | 4.4 |
| 301884 | AA312082 | Hs.105445 | GDNF family receptor alpha 1 | 5.7 |
| 301936 | U79745 | Hs.114924 | solute carrier family 16 (monocarboxylic | 8.6 |
| 301976 | T97905 | | gb: ye54c10.r1 Soares fetal liver spleen | 3.9 |
| 302001 | AB020711 | Hs.278346 | KIAA0904 protein | 7.7 |
| 302067 | BE542706 | Hs.222399 | CEGP1 protein | 7.3 |
| 302094 | AW749321 | Hs.6786 | ESTs | 3.3 |
| 302099 | AL049670 | Hs.137576 | ribosomal protein L34 pseudogene 1 | 4.2 |
| 302145 | NM_003613 | Hs.151407 | cartilage intermediate layer protein, nu | 7.9 |
| 302235 | AL049987 | Hs.166361 | Homo sapiens mRNA; cDNA DKFZp564F112 (fr | 5.6 |
| 302276 | AW057736 | Hs.323910 | HER2 receptor tyrosine kinase (c-erb-b2, | 5.4 |
| 302290 | AA179949 | Hs.175563 | Homo sapiens mRNA; cDNA DKFZp564N0763 (f | 34.1 |
| 302372 | AL117406 | Hs.200102 | ATP-binding cassette transporter MRP8 | 6.7 |
| 302378 | AL109712 | Hs.296506 | Homo sapiens mRNA full length insert cDN | 4 |
| 302384 | AI678059 | Hs.202676 | synaptonemal complex protein 2 | 4.3 |
| 302385 | AJ224172 | Hs.204096 | lipophilin B (uteroglobin family member) | 13.8 |
| 302680 | AW192334 | Hs.38218 | ESTs | 9.6 |
| 302830 | AI038997 | Hs.132921 | ESTs | 5 |
| 302857 | AF282265 | Hs.44836 | inner centromere protein antigens (135 kD | 3.4 |
| 302892 | AW176909 | Hs.42346 | calcineurin-binding protein calsarcin-1 | 3.4 |
| 302970 | W05608 | Hs.312679 | ESTs, Weakly similar to A49019 dynein he | 5.1 |
| 303271 | AA652687 | Hs.96151 | Human DNA sequence from clone RP5-1103G7 | 3.7 |
| 303289 | AL121460 | Hs.272673 | hypothetical protein FLJ20508 | 4.1 |
| 303357 | AW006352 | Hs.159643 | ESTs, Weakly similar to T32554 hypotheti | 4.2 |
| 303540 | AA355607 | Hs.309490 | ESTs, Weakly similar to putative WHSC1 p | 4.3 |
| 303563 | AA367699 | Hs.10082 | potassium intermediate/small conductance | 3.3 |
| 303642 | AW299459 | | gb: xs50d08.x1 NCI_CGAP_Kid11 Homo sapien | 4.2 |
| 303780 | AI424014 | Hs.18995 | KIAA1304 protein | 3.6 |
| 303797 | AW629759 | | gb: hh70e05.yl NCI_CGAP_GU1 Homo sapiens | 4.9 |
| 303852 | R53434 | Hs.90207 | hypothetical protein MGC11138 | 3.7 |
| 304328 | AA149951 | Hs.62112 | zinc finger protein 207 | 3 |
| 304782 | AA582081 | | gb: nn32h08.s1 NCI_CGAP_Gas1 Homo sapiens | 4.1 |
| 305913 | AA876109 | | gb: nx24h01.s1 NCI_CGAP_GC3 Homo sapiens | 3 |
| 305917 | AA876469 | | gb: oe48b04.s1 NCI_CGAP_Pr25 Homo sapiens | 3.1 |
| 307010 | AI140014 | | gb: qa68f09.x1 Soares_fetal_heart_NbHH19W | 3.5 |
| 307041 | AI144243 | | gb: qb85b12.x1 Soares_fetal_heart_NbHH19W | 3.9 |
| 308106 | AI476803 | | gb: tj77e12.x1 Soares_NSF_F8_9W_OT_PA_P_S | 4.3 |
| 308307 | AI581398 | Hs.172928 | collagen, type I, alpha 1 | 4.6 |
| 308615 | AK000142 | Hs.101774 | hypothetical protein FLJ23045 | 4.4 |
| 309177 | AI951118 | Hs.326736 | Homo sapiens breast cancer antigen NY-BR | 17.3 |
| 309328 | AW024348 | Hs.233191 | EST, Weakly similar to A27217 glucose tr | 3.2 |
| 309574 | AW168083 | | gb: xg59g04.x1 NCI_CGAP_Ut4 Homo sapiens | 3.1 |
| 309583 | AW170035 | Hs.326736 | Homo sapiens breast cancer antigen NY-BR | 57.6 |
| 310064 | AI199712 | Hs.148486 | ESTs, Weakly similar to 1917210A Pro/Arg | 4.6 |
| 310098 | AI685841 | Hs.161354 | ESTs | 3.6 |
| 310438 | AW022192 | Hs.200197 | ESTs | 4.6 |
| 310683 | AI939456 | Hs.160870 | ESTs | 3.2 |
| 310727 | AK000703 | Hs.323822 | Homo sapiens mRNA for KIAA1551 protein, | 3.6 |
| 310781 | AI380797 | Hs.158992 | ESTs | 10.2 |
| 310895 | AI955121 | Hs.165724 | N-acetylgalactosamine-4-O-sulfotransfera | 3.4 |
| 310955 | AI476732 | Hs.263912 | ESTs | 10.9 |
| 311117 | AI671439 | Hs.196029 | Homo sapiens mRNA for KIAA1657 protein, | 3.1 |
| 311166 | AI821005 | Hs.118599 | ESTs | 10.8 |
| 311237 | AA641098 | Hs.208809 | ESTs, Moderately similar to ALU1_HUMAN A | 4.3 |
| 311465 | AI758660 | Hs.206132 | ESTs | 4.4 |
| 311587 | AI828254 | Hs.271019 | ESTs, Weakly similar to A47582 B-cell gr | 5.1 |
| 311598 | AW023595 | Hs.232048 | ESTs | 5.8 |
| 311774 | AA700870 | Hs.14304 | ESTs | 3.3 |
| 311785 | AI056769 | Hs.133512 | ESTs | 3.9 |
| 311872 | R12375 | Hs.194600 | ESTs | 3.3 |
| 311889 | AA767342 | Hs.122483 | ESTs, Weakly similar to PSF_HUMAN PTB-AS | 3 |
| 311913 | AI358522 | Hs.270188 | ESTs | 3 |
| 311923 | T60843 | Hs.189679 | ESTs | 5.6 |
| 311935 | AA216387 | | gb: nc16b02.s1 NCI_CGAP_Pr1 Homo sapiens | 5.2 |
| 312019 | AA373630 | Hs.188750 | ESTs | 3 |
| 312021 | AA759263 | Hs.14041 | ESTs | 3.4 |
| 312067 | T78968 | Hs.14411 | ESTs | 3.5 |
| 312090 | T80177 | Hs.118064 | similar to rat nuclear ubiquitous casein | 3.8 |
| 312147 | AI633744 | Hs.195648 | ESTs, Weakly similar to I38022 hypotheti | 4.4 |
| 312153 | BE261944 | Hs.118625 | hexokinase 1 | 5.2 |
| 312168 | T92251 | Hs.198882 | ESTs | 3.3 |
| 312182 | T94344 | Hs.326263 | ESTs | 3.3 |
| 312187 | AA700439 | Hs.188490 | ESTs | 3.4 |
| 312199 | AW438602 | Hs.191179 | ESTs | 3.9 |
| 312219 | H73505 | Hs.117874 | ESTs | 4 |
| 312226 | AA315703 | Hs.199993 | ESTs, Weakly similar to ALUB_HUMAN !!!! | 4.9 |
| 312299 | AA972712 | Hs.269737 | ESTs | 5.7 |
| 312544 | AA516420 | Hs.183526 | ESTs, Weakly similar to I38022 hypotheti | 6.3 |
| 312638 | AW439195 | Hs.256880 | ESTs, Weakly similar to S65657 alpha-1C- | 4.9 |
| 312826 | AW291545 | Hs.185018 | ESTs | 4.9 |
| 312837 | AW292286 | Hs.255058 | ESTs | 4.4 |
| 312980 | AA497043 | Hs.115685 | ESTs | 3.1 |
| 313070 | AI422023 | Hs.161338 | ESTs | 4.3 |
| 313079 | N76497 | Hs.1787 | proteolipid protein 1 (Pelizaeus-Merzbac | 3.3 |
| 313089 | AF026944 | Hs.293797 | ESTs | 5.8 |
| 313096 | AW073310 | Hs.163533 | Homo sapiens cDNA FLJ14142 fis, clone MA | 4.5 |
| 313126 | AA746503 | Hs.283313 | ESTs | 10 |
| 313166 | AI801098 | Hs.151500 | ESTs | 3.5 |
| 313197 | AW979008 | Hs.222487 | ESTs | 3.3 |
| 313280 | AW960454 | Hs.222830 | ESTs | 4.7 |
| 313325 | AI420611 | Hs.127832 | ESTs | 3.4 |
| 313328 | AW449211 | Hs.105445 | GDNF family receptor alpha 1 | 12.4 |
| 313352 | AW150945 | Hs.144758 | ESTs | 4.1 |
| 313385 | AI032087 | Hs.269819 | ESTs | 3 |
| 313393 | AI674685 | Hs.200141 | ESTs | 5.2 |
| 313417 | AA741151 | Hs.137323 | ESTs | 3.5 |
| 313434 | W92070 | | gb: zh48g05.r1 Soares_fetal_liver_spleen_{—} | 3.7 |
| 313569 | AI273419 | Hs.135146 | hypothetical protein FLJ13984 | 3 |
| 313591 | AA046309 | | gb: zf12f01.s1 Soares_fetal_heart_NbHH19W | 5.6 |
| 313615 | AI540978 | Hs.301997 | hypothetical protein FLJ13033 | 3.2 |
| 313915 | C18863 | Hs.163443 | Homo sapiens cDNA FLJ11576 fis, clone HE | 26.3 |
| 313975 | AW175896 | Hs.65114 | keratin 18 | 3 |
| 313979 | AI535895 | Hs.221024 | ESTs | 4.9 |
| 313997 | AV657317 | Hs.288649 | hypothetical protein MGC3077 | 3.9 |
| 314043 | AA827082 | Hs.291872 | ESTs | 3.1 |
| 314078 | AW129357 | Hs.329700 | ESTs | 8.3 |
| 314097 | AA648744 | Hs.269493 | ESTs | 6.6 |
| 314121 | AI732083 | Hs.187619 | ESTs | 6.2 |
| 314129 | AA228366 | Hs.115122 | ESTs | 4 |
| 314138 | AA740616 | | gb: ny97f11.s1 NCI_CGAP_GCB1 Homo sapiens | 5.9 |
| 314236 | AA743396 | Hs.189023 | ESTs | 3.1 |
| 314244 | AL036450 | Hs.103238 | ESTs | 4 |
| 314305 | AI280112 | Hs.125232 | Homo sapiens cDNA FLJ13266 fis, clone OV | 8 |
| 314306 | AI697901 | Hs.192425 | ESTs | 3.7 |
| 314322 | AA907153 | Hs.190060 | ESTs | 3.3 |
| 314394 | AW961597 | Hs.130816 | ESTs, Moderately similar to I38022 hypot | 4.2 |
| 314401 | AI660412 | Hs.234557 | ESTs | 3.3 |
| 314465 | AA602917 | Hs.156974 | ESTs | 4.7 |
| 314506 | AA833655 | Hs.206868 | Homo sapiens cDNA FLJ14056 fis, clone HE | 8.5 |
| 314510 | AI204418 | Hs.190080 | ESTs | 4 |
| 314546 | AW007211 | Hs.16131 | hypothetical protein FLJ12876 | 3.4 |
| 314547 | AA399272 | Hs.144341 | ESTs | 6.7 |
| 314558 | AI873274 | Hs.190721 | ESTs | 27.4 |
| 314627 | AA425310 | Hs.155766 | ESTs, Weakly similar to A47582 B-cell gr | 4.4 |
| 314648 | AW979268 | | gb: EST391378 MAGE resequences, MAGP Homo | 4.6 |
| 314691 | AW207206 | Hs.136319 | ESTs | 20.7 |
| 314729 | AA457367 | Hs.191638 | ESTs | 3.6 |
| 314754 | AW026761 | Hs.134374 | ESTs | 3.6 |
| 314814 | BE350122 | Hs.157367 | ESTs, Weakly similar to I78885 serine/th | 4.9 |
| 314864 | AW971198 | Hs.294068 | ESTs | 4.3 |
| 314881 | AI095087 | Hs.152299 | ESTs, Moderately similar to S65657 alpha | 3.7 |
| 314882 | AA828032 | Hs.189076 | ESTs | 3.1 |
| 314981 | AW972359 | Hs.293334 | ESTs | 3 |
| 315006 | AI538613 | Hs.298241 | Transmembrane protease, serine 3 | 10.9 |
| 315021 | AA533447 | Hs.312989 | ESTs | 5.3 |
| 315051 | AW292425 | Hs.163484 | ESTs | 12.9 |
| 315060 | AA551104 | Hs.189048 | ESTs, Moderately similar to ALUC_HUMAN ! | 5.8 |
| 315073 | AW452948 | Hs.257631 | ESTs | 4.2 |
| 315080 | AA744550 | Hs.136345 | ESTs | 3.7 |
| 315175 | AI025842 | Hs.152530 | ESTs | 6 |
| 315183 | AW136134 | Hs.220277 | ESTs | 3.9 |
| 315193 | AI241331 | Hs.131765 | ESTs, Moderately similar to I38937 DNA/R | 4.4 |
| 315196 | AI367347 | Hs.44898 | Homo sapiens clone TCCCTA00151 mRNA sequ | 8.2 |
| 315198 | AI741506 | Hs.165900 | ESTs, Weakly similar to ALU1_HUMAN ALU S | 3.6 |
| 315240 | R38772 | Hs.172619 | myelin transcription factor 1-like | 3.4 |
| 315263 | AW510994 | Hs.220740 | ESTs | 3.4 |
| 315282 | AI222165 | Hs.144923 | ESTs | 4.9 |
| 315296 | AA876905 | Hs.125286 | ESTs | 4 |
| 315368 | AB037745 | Hs.104696 | KIAA1324 protein | 4.7 |
| 315397 | AA218940 | Hs.137516 | fidgetin-like 1 | 3.1 |
| 315489 | AI378817 | Hs.191847 | ESTs | 3.1 |
| 315498 | AA628539 | Hs.116252 | ESTs, Moderately similar to ALU1_HUMAN A | 3.2 |
| 315526 | AI193043 | Hs.128685 | ESTs, Weakly similar to T17226 hypotheti | 4.1 |
| 315530 | AW015415 | Hs.127780 | ESTs | 8.9 |
| 315562 | AA737415 | Hs.152826 | ESTs | 5.5 |
| 315634 | AA837085 | Hs.220585 | ESTs | 6.3 |
| 315647 | AA648983 | Hs.212911 | ESTs | 3.6 |
| 315707 | AI418055 | Hs.161160 | ESTs | 5.1 |
| 315772 | AW515373 | Hs.271249 | Homo sapiens cDNA FLJ13580 fis, clone PL | 3.1 |
| 315850 | AW270550 | Hs.116957 | ESTs | 3.8 |
| 315858 | AA737345 | Hs.294041 | ESTs | 5 |
| 315878 | AA683336 | Hs.189046 | ESTs | 3.1 |
| 315977 | AW865916 | Hs.151206 | ESTs | 4.7 |
| 315978 | AA830893 | Hs.119769 | ESTs | 4.1 |
| 315995 | AI217477 | Hs.194591 | ESTs | 4.1 |
| 316012 | AA764950 | Hs.119898 | ESTs | 7 |
| 316042 | AI469960 | Hs.170698 | ESTs | 4.9 |
| 316052 | AI962796 | Hs.136754 | ESTs | 4.1 |
| 316072 | AW517524 | Hs.135201 | NOD2 protein | 3.2 |
| 316074 | AW975114 | Hs.293273 | ESTs | 3.8 |
| 316100 | AW203986 | Hs.213003 | ESTs | 3.2 |
| 316133 | AI187742 | Hs.125562 | ESTs | 3.7 |
| 316177 | AI904982 | Hs.293102 | ESTs, Moderately similar to ALU1_HUMAN A | 30.7 |
| 316186 | AI433540 | | gb: ti69g05.x1 NCI_CGAP_Kid11 Homo sapien | 3.1 |
| 316244 | AI640761 | Hs.224988 | ESTs | 3.5 |
| 316303 | AA740994 | Hs.209609 | ESTs | 3.8 |
| 316313 | AA741300 | Hs.202599 | ESTs, Weakly similar to I38022 hypotheti | 4.4 |
| 316364 | AA747807 | Hs.149500 | ESTs | 3.2 |
| 316580 | AA938198 | Hs.146123 | poly(A) polymerase gamma | 9.4 |
| 316697 | AW293174 | Hs.252627 | ESTs | 4.4 |
| 316715 | AI440266 | Hs.170673 | ESTs, Weakly similar to T24832 hypotheti | 3 |
| 316868 | AI660898 | Hs.195602 | ESTs | 3.2 |
| 316869 | AI954880 | Hs.134604 | ESTs | 3.2 |
| 316886 | AA836331 | Hs.134981 | ESTs | 4.4 |
| 316897 | AA838114 | Hs.221612 | ESTs | 3.7 |
| 316943 | AW014875 | Hs.137007 | ESTs | 4.6 |
| 317069 | AI732892 | Hs.190489 | ESTs | 5.9 |
| 317194 | AW445167 | Hs.126036 | ESTs | 4.1 |
| 317360 | AI125252 | Hs.126419 | ESTs | 3.5 |
| 317404 | AI806867 | Hs.126594 | ESTs | 5.1 |
| 317452 | AA972965 | Hs.135568 | ESTs | 6.9 |
| 317501 | AI822034 | Hs.137097 | ESTs | 4.6 |
| 317674 | AW294909 | Hs.132208 | ESTs | 4.3 |
| 317803 | AW664964 | Hs.128899 | ESTs | 6.1 |
| 317834 | X56348 | Hs.287270 | ret proto-oncogene (multiple endocrine n | 3.1 |
| 317850 | AI681545 | Hs.152982 | hypothetical protein FLJ13117 | 3.4 |
| 317881 | AI827248 | Hs.224398 | Homo sapiens cDNA FLJ11469 fis, clone HE | 9.6 |
| 317902 | AW102941 | Hs.211265 | ESTs | 4.1 |
| 317916 | AI565071 | Hs.159983 | ESTs | 10.3 |
| 318042 | AW294522 | Hs.149991 | ESTs | 3.1 |
| 318223 | AI077540 | Hs.134090 | ESTs | 3.9 |
| 318327 | AW294013 | Hs.200942 | ESTs | 3 |
| 318332 | AI093930 | Hs.163440 | Homo sapiens cDNA: FLJ21000 fis, clone C | 4.4 |
| 318418 | AF107493 | Hs.118498 | Homo sapiens LUCA-15 protein mRNA, splic | 5.4 |
| 318558 | AW402677 | Hs.146381 | RNA binding motif protein, X chromosome | 4.4 |
| 318625 | AA526235 | Hs.193162 | Homo sapiens cDNA FLJ11983 fis, clone HE | 5.9 |
| 318634 | T49598 | Hs.156832 | ESTs | 4 |
| 318740 | NM_002543 | Hs.77729 | oxidised low density lipoprotein (lectin | 7.3 |
| 318744 | AI793124 | Hs.144479 | ESTs | 17.8 |
| 318781 | F11802 | Hs.6818 | ESTs | 3 |
| 319191 | NM_012391 | Hs.79414 | prostate epithelium-specific Ets transcr | 3.6 |
| 319478 | AI524124 | Hs.270307 | ESTs | 4.6 |
| 319510 | W88532 | Hs.254562 | ESTs | 3.3 |
| 319551 | AA761668 | | gb: nz24c08.s1 NCI_CGAP_GCB1 Homo sapiens | 3.2 |
| 319745 | T79366 | Hs.108258 | actin binding protein; macrophin (microf | 3.3 |
| 319834 | AA071267 | | gb: zm61g01.r1 Stratagene fibroblast (937 | 6.2 |
| 319840 | C19035 | Hs.164259 | ESTs | 3.3 |
| 319977 | AA534222 | | gb: nj21d02.s1 NCI_CGAP_AA1 Homo sapiens | 4.3 |
| 320074 | AA321166 | Hs.278233 | ESTs | 3.4 |
| 320167 | AA984373 | Hs.90790 | Homo sapiens cDNA: FLJ22930 fis, clone K | 4.1 |
| 320187 | T99949 | Hs.303428 | Homo sapiens cDNA FLJ14832 fis, clone OV | 5.3 |
| 320211 | AL039402 | Hs.125783 | DEME-6 protein | 9.2 |
| 320416 | AI026984 | Hs.293662 | ESTs | 3.1 |
| 320588 | U78082 | Hs.167738 | RNA polymerase II transcriptional regula | 3.1 |
| 320635 | N50617 | Hs.80506 | small nuclear ribonucleoprotein polypept | 6.1 |
| 320654 | AI160015 | Hs.118112 | ESTs | 3.5 |
| 320742 | AI601188 | Hs.120910 | ESTs | 3 |
| 320832 | AA214584 | Hs.290167 | ESTs | 3.7 |
| 320915 | AI359144 | Hs.143688 | Homo sapiens cDNA: FLJ23031 fis, clone L | 3.1 |
| 321016 | BE144167 | Hs.49994 | hypothetical protein similar to RNA-bind | 3.3 |
| 321107 | AI732643 | Hs.144151 | ESTs | 12.3 |
| 321171 | AI769410 | Hs.221461 | ESTs | 3.3 |
| 321253 | AA610649 | Hs.333239 | ESTs | 3 |
| 321318 | AB033041 | Hs.137507 | vang (van gogh, Drosophila)-like 2 | 3.9 |
| 321642 | AI432199 | Hs.247084 | ESTs | 3 |
| 321644 | AW975944 | Hs.237396 | ESTs | 11.7 |
| 321683 | AI471598 | Hs.197531 | ESTs | 3.8 |
| 321758 | U29112 | Hs.196151 | ESTs | 4.4 |
| 321811 | D80630 | | gb: HUM091D02B Human fetal brain (TFujiwa | 3.2 |
| 321828 | R59890 | Hs.83623 | nuclear receptor subfamily 1, group I, m | 3.1 |
| 321910 | H67065 | Hs.271530 | ESTs, Weakly similar to ALU7_HUMAN ALU S | 4.7 |
| 321937 | AL049351 | Hs.302058 | Homo sapiens mRNA; cDNA DKFZp566C093 (fr | 3.5 |
| 321978 | N77342 | Hs.21851 | Homo sapiens cDNA FUJ12900 fis, clone NT | 5 |
| 322035 | AL137517 | Hs.334473 | hypothetical protein DKFZp564O1278 | 19 |
| 322136 | AF075083 | | gb: Homo sapiens full length insert cDNA | 3.6 |
| 322258 | BE265745 | Hs.194359 | ESTs, Weakly similar to ALUC_HUMAN !!!! | 3 |
| 322296 | W76326 | | gb: zd60d04.r1 Soares_fetal_heart_NbHH19W | 4.4 |
| 322303 | AI357412 | Hs.157601 | ESTs | 11.5 |
| 322476 | AW963372 | Hs.46677 | PRO2000 protein | 3 |
| 322520 | T55958 | | gb: yb35f05.r1 Stratagene fetal spleen (9 | 3 |
| 322521 | AF147347 | | gb: Homo sapiens full length insert cDNA | 4.2 |
| 322567 | AF155108 | Hs.256150 | Homo sapiens, Similar to RIKEN cDNA 2810 | 4 |
| 322595 | W92147 | Hs.118394 | ESTs | 5.4 |
| 322675 | AA017656 | | gb: ze39h01.r1 Soares retina N2b4HR Homo | 3.1 |
| 322766 | AW068805 | Hs.288467 | Homo sapiens cDNA FLJ12280 fis, clone MA | 5.2 |
| 322818 | AW043782 | Hs.293616 | ESTs | 7.6 |
| 322882 | AW248508 | Hs.279727 | Homo sapiens cDNA FLJ14035 fis, clone HE | 5.9 |
| 322975 | C16391 | | gb: C16391 Clontech human aorta polyA mRN | 16.5 |
| 323091 | AI902456 | Hs.210761 | ESTs, Weakly similar to I38022 hypotheti | 4 |
| 323131 | AK002088 | Hs.270124 | Homo sapiens cDNA FLJ11226 fis, clone PL | 3.3 |
| 323168 | AL120862 | Hs.124165 | programmed cell death 9 (PDCD9) | 6.3 |
| 323244 | AW675572 | Hs.193620 | ESTs | 4.6 |
| 323262 | AL133990 | Hs.190642 | ESTs | 10.5 |
| 323332 | AI829520 | | gb: wI19c06.x1 NCI_CGAP_Ut1 Homo sapiens | 6.2 |
| 323333 | AV651680 | Hs.208558 | ESTs | 4.3 |
| 323335 | AI655499 | Hs.161712 | ESTs | 9.2 |
| 323645 | AW445014 | Hs.197746 | ESTs | 3.1 |
| 323663 | BE081058 | Hs.243023 | ESTs | 4 |
| 323693 | AA317962 | Hs.249721 | ESTs, Moderately similar to PC4259 ferri | 3 |
| 323782 | AW961560 | Hs.97600 | ESTs | 3.2 |
| 323817 | AA410943 | | BMP-R1B | 8.4 |
| 323930 | AL043683 | Hs.8173 | hypothetical protein FLJ10803 | 3.3 |
| 323974 | AI825204 | Hs.211408 | ESTs | 4.5 |
| 324001 | AL044949 | Hs.116298 | ESTs | 4.5 |
| 324036 | AI472078 | Hs.303662 | ESTs | 8.4 |
| 324261 | BE069341 | | gb: QV3-BT0381-270100-073-c08 BT0381 Homo | 49.4 |
| 324285 | AA431159 | Hs.122954 | ESTs | 3 |
| 324296 | AI524039 | Hs.192524 | ESTs | 3 |
| 324305 | AA642007 | Hs.116369 | ESTs | 3.3 |
| 324432 | AA464510 | Hs.152812 | ESTs | 16.5 |
| 324585 | AI823969 | Hs.132678 | ESTs | 3.3 |
| 324598 | AW972227 | Hs.163986 | Homo sapiens cDNA: FLJ22765 fis, clone K | 5 |
| 324603 | AW993522 | Hs.292934 | ESTs | 10.4 |
| 324631 | AA937116 | Hs.293683 | ESTs, Weakly similar to I54374 gene NF2 | 3.3 |
| 324716 | BE169746 | Hs.12504 | likely ortholog of mouse Arkadia | 3.2 |
| 324748 | AW974941 | Hs.292385 | ESTs, Weakly similar to I78885 serine/th | 3 |
| 324771 | AA631739 | Hs.335440 | EST | 3 |
| 324774 | AI031771 | Hs.132586 | ESTs | 4.2 |
| 324823 | AW516704 | Hs.208726 | ESTs | 3.4 |
| 324824 | AI826999 | Hs.224624 | ESTs | 3.1 |
| 324826 | AA704806 | Hs.143842 | ESTs, Weakly similar to 2004399A chromos | 4.4 |
| 324961 | AA613792 | | gb: no97h03.s1 NCI_CGAP_Pr2 Homo sapiens | 3.9 |
| 324987 | AI375572 | Hs.172634 | ESTs | 18.8 |
| 324994 | AI805416 | Hs.213897 | ESTs | 3.3 |
| 325146 | AI064690 | Hs.171176 | ESTs | 4.2 |
| 325372 | | | Phase 2 & 3 Exons | 4.4 |
| 325544 | | | Phase 2 & 3 Exons | 5.7 |
| 327075 | | | Phase 2 & 3 Exons | 3.8 |
| 332798 | | | C22000007: gi\|12314195\|emb\|CAB99338.1\| (A | 4.3 |
| 334223 | | | NM_005080*: Homo sapiens X-box binding pr | 26.2 |
| 334447 | | | NM_012429*: Homo sapiens SEC14 (S. cerevi | 3.9 |
| 335809 | | | NM_014509*: Homo sapiens kraken-like (BK1 | 10.1 |
| 335824 | | | ENSP00000249072*: DJ222E13.1 (N-TERMINAL | 20 |
| 338255 | | | NM_014323*: Homo sapiens zinc finger prot | 9 |
| 409430 | R21945 | Hs.166975 | splicing factor, arginine/serine-rich 5 | 4 |
| 428046 | AW812795 | Hs.155381 | ESTs, Moderately similar to I38022 hypot | 4.6 |
| 432558 | R97268 | Hs.177269 | ESTs | 3.2 |
| 436808 | AA731602 | Hs.120266 | ESTs | 3.9 |
| 448569 | BE382657 | Hs.21486 | signal transducer and activator of trans | 4.1 |
| 453542 | AW836724 | Hs.339660 | Homo sapiens mRNA expressed only in plac | 3.7 |
| | M97935 | | AFFX control: STAT1 | 3.2 |
| | M97935 | | AFFX control: STAT1 | 3 |
| | M55150 | | fumarylacetoacetate | 3 |
| | M13755 | | interferon stimulated protein; 15 kDa | 4.5 |
| | AI052047 | | ESTs | 6.7 |
| | AA252033 | | ESTs; Weakly similar to !!!! ALU SUBFAMILY J | 3.2 |
| | AA401739 | | ESTs | 3.3 |
| | H18459 | | hepatocellular carcinoma associated protein; | 3 |
| | R48744 | | ESTs | 4.2 |
| | M31682 | | inhibin; beta B (activin AB beta polypeptide) | 3 |
| | AA416873 | | ESTs | 3 |
| | D80240 | | HUM5G11A Human fetal brain (TFujiwara) Homo | 4 |
| | R49590 | | ESTs | 3.2 |
| | | | CH22_FGENES.678_5 | 16.8 |
| | | | CH22_FGENES.619_7 | 12.9 |
| | | | CH22_FGENES.619_12 | 11.3 |
| | | | CH22_EM: AC005500.GENSCAN.127 9 | 9.2 |
| | | | CH22_EM: AC005500.GENSCAN.304 2 | 8.5 |
| | | | CH22_FGENES.271_8 | 8.4 |
| | | | CH22_FGENES.619_13 | 8 |
| | | | CH22_FGENES.271_7 | 7.3 |
| | | | CH22_FGENES.617_7 | 7.2 |
| | | | CH.07_hs gi\|6004473 | 7.1 |
| | | | CH22_FGENES.264_1 | 6.8 |
| | X03363 | | HER2 receptor tyrosine kinase (c erbB 2; ERBB2; neu) | 6.6 |
| | | | CH22_FGENES.617_9 | 6.5 |
| | | | CH.07_hs gi\|5868264 | 5.8 |
| | | | CH.19_hs gi\|5867439 | 5.7 |
| | | | CH22_FGENES.6 3 | 5.3 |
| | | | CH.17_hs gi\|5867230 | 5.1 |
| | | | CH.20_hs gi\|6552458 | 5.1 |
| | | | CH22_EM: AC005500.GENSCAN.148 22 | 4.7 |
| | | | CH22_FGENES.669_10 | 4.6 |
| | AA034918 | | KIAA1028 protein | 4.6 |
| | | | CH22_FGENES.48_12 | 4.5 |
| | | | CH22_FGENES.118_2 | 4.5 |
| | AF049569 | | ESTs | 4.4 |
| | M13955 | | multiple UniGene matches | 4.3 |
| | | | CH22_FGENES.619_8 | 4.3 |
| | | | CH22_FGENES.13 7 | 4.3 |
| | HG4126 HT4396 | | Zinc Finger Protein Hzf4 | 4.3 |
| | | | CH22_FGENES.360_3 | 4.3 |
| | | | CH22_FGENES.706_9 | 4.3 |
| | | | CH.21_hs gi\|6531965 | 4.2 |
| | | | CH.17_hs gi\|5867215 | 4.1 |
| | | | CH22_FGENES.669_8 | 4.1 |
| | HG2614 HT2710 | | Collagen, Type Viii, Alpha 1 | 4.1 |
| | | | CH22_FGENES.48_18 | 4.1 |
| | X83535 | | matrix metalloproteinase 14 (membrane inserted) | 4 |
| | | | CH22_FGENES.271_6 | 3.9 |
| | | | CH22_FGENES.617_3 | 3.9 |
| | | | CH22_FGENES.290_8 | 3.8 |
| | HG4716 HT5158 | | Guanosine 5′ Monophosphate Synthase | 3.8 |
| | | | CH22_FGENES.13 5 | 3.8 |
| | | | CH22_FGENES.13 2 | 3.8 |
| | | | CH.14_hs gi\|6682474 | 3.8 |
| | | | CH.02_hs gi\|5867750 | 3.8 |
| | | | CH22_FGENES.617_8 | 3.7 |
| | HG4677 HT5102 | | Oncogene Ret/Ptc2, Fusion Activated | 3.7 |
| | | | CH22_DJ32I10.GENSCAN.23 39 | 3.7 |
| | | | CH22_FGENES.543_20 | 3.7 |
| | | | CH22_EM: AC005500.GENSCAN.96 1 | 3.7 |
| | | | CH22_FGENES.204_2 | 3.5 |
| | | | CH22_FGENES.619_4 | 3.5 |
| | | | CH.16_hs gi\|5867087 | 3.5 |
| | AA714311 | | EST cluster (not in UniGene) | 3.4 |
| | | | CH22_EM: AC005500.GENSCAN.149 9 | 3.4 |
| | | | CH22_EM: AC005500.GENSCAN.421 5 | 3.4 |
| | | | CH22_FGENES.13 4 | 3.3 |
| | | | CH.07_hs gi\|6004478 | 3.3 |
| | | | CH22_FGENES.360_1 | 3.3 |
| | HG2465 HT4871 | | Dna Binding Protein Ap 2, Alt. Splice 3 | 3.3 |
| | | | CH22_FGENES.6_2 | 3.3 |
| | | | CH22_C20H12.GENSCAN.16 2 | 3.2 |
| | | | CH22_C65E1.GENSCAN.8 1 | 3.2 |
| | AA707750 | | ESTs; Weakly similar to cis Golgi matrix | 3.1 |
| | | | CH22_FGENES.307_4 | 3.1 |
| | | | CH22_EM: AC005500.GENSCAN.248 14 | 3.1 |
| | | | CH.06_hs gi\|5902482 | 3.1 |
| | | | CH22_FGENES.669_5 | 3.1 |
| | | | CH22_DJ32I10.GENSCAN.19 8 | 3.1 |
| | | | CH22_FGENES.527_6 | 3.1 |
| | | | CH22_FGENES.330_10 | 3.1 |
| | | | CH22_FGENES.14 2 | 3.1 |
| | AA976074 | | ESTs | 3 |
| | | | CH22_FGENES.226 7 | 3 |
| | | | CH22_FGENES.13 3 | 3 |
| | | | CH22_EM: AC005500.GENSCAN.209 12 | 3 |
| | | | CH22_FGENES.271_3 | 3 |

Table 17 shows accession numbers representing 759 sequences of breast cancer genes or fragments thereof encoding breast cancer modulating proteins. For each overexpressed gene identified, a ratio of the relative amount of expression in breast tumors versus normal breast tissue.
Pkey: Unique Eos probeset identifier number
ExAccn: Exemplar Accession number, Genbank accession number
UnigeneID: Unigene number
Unigene Title: Unigene gene title
R1: Ratio of tumor to normal breast tissue

TABLE 17A
Table 17A shows the accession numbers for those pkeys lacking unigeneID's for Table 17.
For each probeset, we have listed the gene cluster number from which the oligonucleotides
were designed. Gene clusters were compiled using sequences derived from Genbank ESTs
and mRNAs. These sequences were clustered based on sequence similarity using Clustering
and Alignment Tools (DoubleTwist, Oakland California). The Genbank accession numbers
for sequences comprising each cluster are listed in the “Accession” column.

| Pkey | CAT number | Accession |
|---|---|---|
| 116845 | 393481_1 | AA649530 AA659316 H64973 |
| 103207 | 30635_4 | X72790 |
| 126257 | 182217_1 | N99638 AW973750 AA328271 |
| | | H90994 AA558020 AA234435 N59599 R94815 |
| 102791 | 37186_1 | AF080229 AF080231 AF080230 |
| | | AF080232 AF080233 AF080234 BE550633 AI636743 |
| | | AW614951 BE467547 AI680833 |
| | | AI633818 N29986 U87592 U87593 U87590 U87591 S46404 |
| | | U87587 AA463992 AW206802 |
| | | AI970376 AI583718 AI672574 |
| | | N25695 AW665466 AI818326 AA126128 AI480345 AW013827 |
| | | AA248638 AI214968 AA204735 |
| | | AA207155 AA206262 AA204833 |
| | | AW003247 AW496808 AI080480 AI631703 AI651023 AI867418 |
| | | AW818140 AA502500 |
| | | AI206199 AI671282 AI352545 BE501030 |
| | | AI652535 BE465762 AA206331 AW451866 AA471088 |
| | | AA206342 AA204834 AA206100 AW021661 |
| | | AA332922 N66048 |
| | | AA703396 H92278 AW139734 H92683 U87589 U87595 H69001 |
| | | U87594 BE466420 AI624817 |
| | | BE466611 AI206344 AA574397 |
| | | AA348354 AI493192 |
| 126872 | 142696_1 | AW450979 AA136653 AA136656 |
| | | AW419381 AA984358 AA492073 |
| | | BE168945 AA809054 AW238038 BE011212 BE011359 |
| | | BE011367 BE011368 BE011362 BE011215 6E011365 BE011363 |
| 112631 | 1746257_1 | R82040 R70934 |
| 120742 | 176835_1 | AA225084 AA302713 |
| 106864 | 324239_1 | AI311928 AA936030 T51931 AA609816 |
| | | AA487195 AA664207 |
| 109700 | genbank_F09609 | F09609 |
| 111532 | genbank_R08440 | R08440 |
| 113938 | genbank_W81598 | W81598 |
| 113947 | genbank_W84768 | W84768 |
| 124357 | genbank_N22401 | N22401 |
| 108733 | 504187_1 | AA121022 AA126422 |
| 112303 | genbank_R54797 | R54797 |
| 322136 | 46802_1 | AF075083 H52291 H52528 |
| 322296 | 47334_1 | W76326 AF086341 W72300 |
| 321811 | 1527481_1 | D80630 D80896 D80895 |
| 314648 | 293660_1 | AW979268 AA878419 AA431342 AA431628 |
| 322520 | 38916_1 | T55958 T57205 AF147346 |
| 322521 | 38917_1 | AF147347 T55426 T55503 |
| 322675 | 86787_1 | AA017656 AA017374 AA019761 |
| 323332 | 179142_1 | AI829520 AI791832 AA228414 AI791823 |
| | | AA229211 AA229315 |
| 316186 | 425440_1 | AI433540 AA728984 AA804981 |
| 322975 | 1510563_1 | C16391 C16413 |
| 324261 | 273265_1 | BE069341 AW748403 AL044891 AI908240 AA393080 |
| 323817 | 233566_1 | AA410943 AW948953 AA334202 AA332882 |
| 301976 | 128835_1 | T97905 AA101672 |
| 324961 | 376239_1 | AA613792 AW182329 T05304 AW858385 |
| 303642 | 284260_1 | AW299459 AA417112 |
| 303797 | 386364_1 | AW629759 AW749955 AA633408 AI651005 |
| 319551 | 357371_1 | AA761668 AA573621 R92814 R09670 |
| 311935 | 174129_1 | AA216387 T63548 M228676 |
| 319834 | 112523_1 | AA071267 T65940 T64515 AA071334 |
| 319977 | 345248_1 | AA534222 AA632632 T81234 |
| 314138 | 179960_1 | AA740616 AA654854 AA229923 |
| 313591 | 103087_1 | AA046309 AI263500 AA046397 |
| 308106 | AI476803 | |
| 338255 | CH22_6856FG_LINK_EM: AC00 | |
| 335809 | CH22_3181FG_617_6_LINK_EM | |
| 335824 | CH22_3197FG_619_11_LINK_E | |
| 307010 | AI140014 | |
| 307041 | AI144243 | |
| 305913 | AA876109 | |
| 305917 | AA876469 | |
| 309574 | AW168083 | |
| 325372 | c12_hs | |
| 325544 | c12_hs | |
| 332798 | CH22_14FG_6_5_LINK_C4G1.G | |
| 334223 | CH22_1507FG_360_4_LINK_EM | |
| 327075 | c21_hs | |
| 334447 | CH22_1746FG_387_7_LINK_EM | |
| 304782 | AA582081 | |
| 313434 | 441798_1 W92070 AW019952 W92053 | |

Pkey: Unique Eos probeset identifier number
CAT number: Gene cluster number
Accession: Genbank accession numbers

TABLE 17B
Table 17B shows the genomic positioning for those pkeys
lacking unigene ID's and accession numbers in Table 17. For each
predicted exon, we have listed the genomic sequence source used
for prediction. Nucleotide locations of each predicted exon
are also listed.

| Pkey | Ref | Strand | Nt_position |
|---|---|---|---|
| 334447 | Dunham, I. et.al. | Plus | 14308764-14308824 |
| 335809 | Dunham, I. et.al. | Plus | 26310772-26310909 |
| 335824 | Dunham, I. et.al. | Plus | 26376860-26376942 |
| 332798 | Dunham, I. et.al. | Minus | 232147-231974 |
| 334223 | Dunham, I. et.al. | Minus | 12734365-12734269 |
| 338255 | Dunham, I. et.al. | Minus | 15242294-15242231 |
| 325372 | 5866920 | Minus | 1117061-1117304 |
| 325544 | 6682452 | Plus | 171228-171286 |
| 327075 | 6531965 | Plus | 4041318-4041431 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 18
Table 2 from BRCA 014 P

| Pkey | ExAccn | UnigeneID | Unigene Title | R1 |
|---|---|---|---|---|
| 101378 | BE563085 | Hs.833 | interferon-stimulated protein, 15 kDa | 5.3 |
| 101530 | M29874 | Hs.1360 | cytochrome P450, subfamily IIB (phenobar | 9 |
| 101767 | M81057 | Hs.180884 | carboxypeptidase B1 (tissue) | 12 |
| 101878 | M97815 | Hs.183650 | cellular retinoic acid-binding protein 2 | 6.5 |
| 103010 | X52509 | Hs.161640 | tyrosine aminotransferase | 12.4 |
| 104115 | AF183810 | Hs.26102 | opposite strand to trichorhinophalangeal | 7.6 |
| 104825 | AA035613 | Hs.141883 | ESTs | 6.9 |
| 107105 | AW963419 | Hs.155223 | stanniocalcin 2 | 5.3 |
| 108819 | AA011449 | Hs.271627 | ESTs | 6.1 |
| 112287 | AB033064 | Hs.334806 | KIAA1238 protein | 7.3 |
| 112561 | AI791493 | Hs.129873 | ESTs, Weakly similar to A36036 cytochrom | 8.2 |
| 112637 | R82331 | Hs.164599 | ESTs | 5.4 |
| 113206 | BE262470 | Hs.241471 | RNB6 | 6.2 |
| 113970 | W27249 | Hs.8109 | hypothetical protein FLJ21080 | 6.9 |
| 114965 | AI733881 | Hs.72472 | BMP-R1B | 10.1 |
| 118925 | N92293 | Hs.206832 | ESTs, Moderately similar to ALU8_HUMAN A | 19.7 |
| 119905 | AW449064 | Hs.119571 | collagen, type III, alpha 1 (Ehlers-Danl | 8.4 |
| 121611 | M31669 | Hs.1735 | inhibin, beta B (activin AB beta polypep | 5.6 |
| 129301 | AF182277 | Hs.330780 | cytochrome P450, subfamily IIB (phenobar | 6.2 |
| 133976 | AI908165 | Hs.169946 | GATA-binding protein 3 (T-cell receptor | 6.2 |
| 134731 | D89377 | Hs.89404 | msh (Drosophila) homeo box homolog 2 | 5.8 |
| 300254 | AW183618 | Hs.55610 | solute carrier family 30 (zinc transport | 9.9 |
| 301884 | AA312082 | Hs.105445 | GDNF family receptor alpha 1 | 5.7 |
| 302001 | AB020711 | Hs.278346 | KIAA0904 protein | 7.7 |
| 302067 | BE542706 | Hs.222399 | CEGP1 protein | 7.3 |
| 302276 | AW057736 | Hs.323910 | HER2 receptor tyrosine kinase (c-erb-b2, | 5.4 |
| 302290 | AA179949 | Hs.175563 | Homo sapiens mRNA; cDNA DKFZp564NO763 (f | 34.1 |
| 302372 | AL117406 | Hs.200102 | ATP-binding cassette transporter MRP8 | 6.7 |
| 302385 | AJ224172 | Hs.204096 | lipophilin B (uteroglobin family member) | 13.8 |
| 309177 | AI951118 | Hs.326736 | Homo sapiens breast cancer antigen NY-BR | 17.3 |
| 309583 | AW170035 | Hs.326736 | Homo sapiens breast cancer antigen NY-BR | 57.6 |
| 310781 | AI380797 | Hs.158992 | ESTs | 10.2 |
| 311166 | AI821005 | Hs.118599 | ESTs | 10.8 |
| 311935 | AA216387 | | gb: nc16b02.s1 NCI_CGAP_Pr1 Homo sapiens | 5.2 |
| 312153 | BE261944 | Hs.118625 | hexokinase 1 | 5.2 |
| 313328 | AW449211 | Hs.105445 | GDNF family receptor alpha 1 | 12.4 |
| 313915 | C18863 | Hs.163443 | Homo sapiens cDNA FLJ11576 fis, clone HE | 26.3 |
| 314097 | AA648744 | Hs.269493 | ESTs | 6.6 |
| 314138 | AA740616 | | gb: ny97f11.s1 NCI_CGAP_GCB1 Homo sapiens | 5.9 |
| 314506 | AA833655 | Hs.206868 | Homo sapiens cDNA FLJ14056 fis, clone HE | 8.5 |
| 314558 | AI873274 | Hs.190721 | ESTs | 27.4 |
| 314691 | AW207206 | Hs.136319 | ESTs | 20.7 |
| 315006 | AI538613 | Hs.298241 | Transmembrane protease, serine 3 | 10.9 |
| 315021 | AA533447 | Hs.312989 | ESTs | 5.3 |
| 315051 | AW292425 | Hs.163484 | ESTs | 12.9 |
| 315060 | AA551104 | Hs.189048 | ESTs, Moderately similar to ALUC_HUMAN! | 5.8 |
| 315196 | AI367347 | Hs.44898 | Homo sapiens clone TCCCTA00151 mRNA sequ | 8.2 |
| 315530 | AW015415 | Hs.127780 | ESTs | 8.9 |
| 315634 | AA837085 | Hs.220585 | ESTs | 6.3 |
| 316012 | AA764950 | Hs.119898 | ESTs | 7 |
| 316177 | AI904982 | Hs.293102 | ESTs, Moderately similar to ALU1_HUMAN A | 30.7 |
| 316580 | AA938198 | Hs.146123 | poly(A) polymerase gamma | 9.4 |
| 317803 | AW664964 | Hs.128899 | ESTs | 6.1 |
| 317881 | AI827248 | Hs.224398 | Homo sapiens cDNA FLJ11469 fis, clone HE | 9.6 |
| 318740 | NM_002543 | Hs.77729 | oxidised low density lipoprotein (lectin | 7.3 |
| 318744 | AI793124 | Hs.144479 | ESTs | 17.8 |
| 320211 | AL039402 | Hs.125783 | DEME-6 protein | 9.2 |
| 321107 | AI732643 | Hs.144151 | ESTs | 12.3 |
| 321644 | AW975944 | Hs.237396 | ESTs | 11.7 |
| 321978 | N77342 | Hs.21851 | Homo sapiens cDNA FLJ12900 fis, clone NT | 5 |
| 322035 | AL137517 | Hs.334473 | hypothetical protein DKFZp564O1278 | 19 |
| 322766 | AW068805 | Hs.288467 | Homo sapiens cDNA FLJ12280 fis, clone MA | 5.2 |
| 322818 | AW043782 | Hs.293616 | ESTs | 7.6 |
| 322975 | C16391 | | gb: C16391 Clontech human aorta polyA mRN | 16.5 |
| 323262 | AL133990 | Hs.190642 | ESTs | 10.5 |
| 323332 | AI829520 | | gb: Wl19c06.x1 NCI_CGAP_Ut1 Homo sapiens | 6.2 |
| 323817 | AA410943 | | BMP-R1B | 8.4 |
| 324261 | BE069341 | | gb: QV3-BT0381-270100-073-c08 BT0381 Homo | 49.4 |
| 324432 | AA464510 | Hs.152812 | ESTs | 16.5 |
| 324598 | AW972227 | Hs.163986 | Homo sapiens cDNA: FLJ22765 fis, clone K | 5 |
| 324603 | AW993522 | Hs.292934 | ESTs | 10.4 |
| 324987 | AI375572 | Hs.172634 | ESTs | 18.8 |
| 325544 | | | Phase 2 & 3 Exons | 5.7 |
| 330388 | | Hs.46 | HER2 receptor tyrosine kinase (c-erb-b2, | 6.6 |
| 334223 | | | NM_005080*: Homo sapiens X-box binding pr | 26.2 |
| 335809 | | | NM_014509*: Homo sapiens kraken-like (BK1 | 10.1 |
| 335824 | | | ENSP00000249072*: DJ222E13.1 (N-TERMINAL | 20 |
| | AI052047 | | ESTs; Weakly similar to CYTOCHROME P450 | 6.7 |
| | R72427 | | CH22_EM: AC005500.GENSCAN.127 9 | 5.5 |
| | | | CH22_FGENES.619_13 | 9.2 |
| | | | CH22_FGENES.617_9 | 8 |
| | | | CH22_FGENES.271_7 | 6.5 |
| | | | CH22_FGENES.619_7 | 7.3 |
| | | | CH22_FGENES.271_8 | 12.9 |
| | | | CH22_FGENES.619_12 | 8.4 |
| | | | CH22_EM: AC005500.GENSCAN.304 2 | 11.3 |
| | | | CH.07_hs gi\|6004473 | 8.5 |
| | | | CH22_FGENES.617_7 | 7.1 |
| | | | CH22_FGENES.678_5 | 7.2 |
| | | | CH22_FGENES.678_5 | 16.8 |

Table 18 shows genes with atleast five times the expression in breast tumor tissue than is expressed in normal body tissues.
Pkey: Unique Eos probeset identifier number
ExAccn: Exemplar Accession number, Genbank accession number
UnigeneID: Unigene number
Unigene Title: Unigene gene title
R1: Ratio of tumor to normal body tissue

TABLE 18A
Table 18A shows the accession numbers for those pkeys
lacking unigeneID's for Table 18. For each probeset,
we have listed the gene cluster number from which the
oligonucleotides were designed. Gene clusters were compiled
using sequences derived from Genbank ESTs and mRNAs. These
sequences were clustered based on sequence similarity
using Clustering and Alignment Tools (DoubleTwist, Oakland
California). The Genbank accession numbers for sequences
comprising each cluster are listed in the “Accession” column.

| Pkey | CAT number | Accession |
|---|---|---|
| 323332 | 179142_1 | AI829520 AI791832 AA228414 AI791823 |
| | | AA229211 AA229315 |
| 322975 | 1510563_1 | C16391 C16413 |
| 324261 | 273265_1 | BE069341 AW748403 AL044891 AI908240 |
| | | AA393080 |
| 323817 | 233566_1 | AA410943 AW948953 AA334202 |
| | | AA332882 |
| 311935 | 174129_1 | AA216387 T63548 AA228676 |
| 314138 | 179960_1 | AA740616 AA654854 AA229923 |
| 335809 | CH22_3181FG_{—} | |
| | 617_6_{—} | |
| | LINK_EM | |
| 335824 | CH22_3197FG_{—} | |
| | 619_11_{—} | |
| | LINK_E | |
| 325544 | c12_hs | |
| 334223 | CH22_1507FG_{—} | |
| | 360_4_{—} | |
| | LINK_EM | |

Pkey: Unique Eos probeset identifier number
CAT number: Gene cluster number
Accession: Genbank accession numbers

TABLE 18B
Table 18B shows the genomic positioning for those pkeys
lacking unigene ID's and accession numbers in Table 18.
For each predicted exon, we have listed the genomic
sequence source used for prediction. Nucleotide locations
of each predicted exon are also listed.

| Pkey | Ref | Strand | Nt_position |
|---|---|---|---|
| 335809 | Dunham, I. et.al. | Plus | 26310772-26310909 |
| 335824 | Dunham, I. et.al. | Plus | 26376860-26376942 |
| 334223 | Dunham, I. et.al. | Minus | 12734365-12734269 |
| 325544 | 6682452 | Plus | 171228-171286 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 19
1045 GENES UP-REGULATED IN BREAST CANCER COMPARED TO
NORMAL ADULT TISSUES

| Pkey | ExAccn | UnigeneID | UnigeneTitle | R1 |
|---|---|---|---|---|
| 408591 | AF015224 | Hs.46452 | mammaglobin 1 | 137.6 |
| 406964 | M21305 | | gb: Human alpha satellite and satellite 3 | 71.0 |
| 400291 | AA401369 | Hs.190721 | ESTs | 68.4 |
| 407277 | AW170035 | Hs.326736 | Homo sapiens breast cancer antigen NY-BR | 54.2 |
| 449746 | AI668594 | Hs.176588 | ESTs, Weakly similar to CP4Y_HUMAN CYTOC | 46.4 |
| 426878 | BE069341 | | gb: QV3-BT0381-270100-073-c08 BT0381 Homo | 44.8 |
| 400292 | AA250737 | Hs.72472 | BMP-R1B | 37.4 |
| 427585 | D31152 | Hs.179729 | collagen, type X, alpha 1 (Schmid metaph | 32.9 |
| 408045 | AW138959 | Hs.245123 | ESTs | 31.9 |
| 407178 | AA195651 | Hs.104106 | ESTs | 30.4 |
| 407377 | C16391 | | gb: C16391 Clontech human aorta polyA mRN | 27.7 |
| 450705 | U90304 | Hs.25351 | iroquois homeobox protein 5 | 24.8 |
| 407212 | AA412108 | Hs.269350 | ESTs | 22.0 |
| 428848 | NM_000230 | Hs.194236 | leptin (murine obesity homolog) | 21.9 |
| 404561 | | | trichorhinophalangeal syndrome I (TRPS1) | 21.8 |
| 407980 | AA046309 | | gb: zf12f01.s1 Soares_fetal_heart_NbHH19W | 19.8 |
| 447350 | AI375572 | Hs.172634 | ESTs | 17.3 |
| 450375 | AA009647 | Hs.8850 | a disintegrin and metalloproteinase doma | 16.6 |
| 422109 | S73265 | Hs.1473 | gastrin-releasing peptide | 16.5 |
| 435496 | AW840171 | Hs.265398 | ESTs, Weakly similar to transformation-r | 16.0 |
| 453160 | AI263307 | Hs.239884 | H2B histone family, member L | 15.8 |
| 420813 | X51501 | Hs.99949 | prolactin-induced protein | 15.8 |
| 415989 | AI267700 | Hs.317584 | ESTs | 15.5 |
| 422505 | AL120862 | Hs.124165 | programmed cell death 9 (PDCD9) | 14.8 |
| 424399 | AI905687 | Hs.2533 | aldehyde dehydrogenase 9 family, member | 14.5 |
| 423575 | C18863 | Hs.163443 | Homo sapiens cDNA FLJ11576 fis, clone HE | 13.7 |
| 429441 | AJ224172 | Hs.204096 | lipophilin B (uteroglobin family member) | 13.6 |
| 431474 | AL133990 | Hs.190642 | ESTs | 13.5 |
| 448595 | AB014544 | Hs.21572 | KIAA0644 gene product | 13.0 |
| 427217 | AA399272 | Hs.144341 | ESTs | 12.8 |
| 402578 | | | C1001134: gi\|2117372\|pir\|\|I65981 fatty ac | 12.6 |
| 422805 | AA436989 | Hs.121017 | H2A histone family, member A | 12.2 |
| 424634 | NM_003613 | Hs.151407 | cartilage intermediate layer protein, nu | 12.0 |
| 456207 | AA193450 | | gb: zr40e07.r1 Soares_NhHMPu_S1 Homo sapi | 11.9 |
| 424086 | AI351010 | Hs.102267 | lysyl oxidase | 11.9 |
| 459587 | AA031956 | | gb: zk15e04.s1 Soares_pregnant_uterus_NbH | 11.5 |
| 416208 | AW291168 | Hs.41295 | ESTs, Weakly similar to MUC2_HUMAN MUCIN | 11.5 |
| 429170 | NM_001394 | Hs.2359 | dual specificity phosphatase 4 | 11.5 |
| 407276 | AI951118 | Hs.326736 | Homo sapiens breast cancer antigen NY-BR | 11.4 |
| 434377 | AW137148 | Hs.306593 | Homo sapiens cDNA FLJ11382 fis, clone HE | 11.3 |
| 448390 | AL035414 | Hs.21068 | hypothetical protein | 11.1 |
| 452401 | NM_007115 | Hs.29352 | tumor necrosis factor, alpha-induced pro | 11.0 |
| 421037 | AI684808 | Hs.197653 | programmed cell death 9 (PDCD9) | 10.9 |
| 452461 | N78223 | Hs.108106 | transcription factor | 10.7 |
| 443348 | AW873596 | Hs.182278 | calmodulin 2 (phosphorylase kinase, delt | 10.6 |
| 421155 | H87879 | Hs.102267 | lysyl oxidase | 10.5 |
| 402606 | | | NM_024626: Homo sapiens hypothetical prot | 10.4 |
| 409269 | AA576953 | Hs.22972 | hypothetical protein FLJ13352 | 10.4 |
| 447268 | AI370413 | Hs.36563 | hypothetical protein FLJ22418 | 10.3 |
| 447033 | AI357412 | Hs.157601 | ESTs | 10.2 |
| 400295 | W72838 | Hs.2533 | aldehyde dehydrogenase 9 family, member | 10.1 |
| 424905 | NM_002497 | Hs.153704 | NIMA (never in mitosis gene a)-related k | 10.1 |
| 432441 | AW292425 | Hs.163484 | ESTs | 9.9 |
| 427365 | AI873274 | Hs.190721 | ESTs | 9.9 |
| 438950 | H23789 | Hs.144530 | EST | 9.8 |
| 422835 | BE218705 | Hs.121378 | metallothionein-like 5, testis-specific | 9.7 |
| 425692 | D90041 | Hs.155956 | N-acetyltransferase 1 (arylamine N-acety | 9.7 |
| 411869 | W20027 | Hs.23439 | ESTs | 9.6 |
| 439820 | AL360204 | Hs.283853 | Homo sapiens mRNA full length insert cDN | 9.6 |
| 445730 | AI624342 | Hs.170042 | ESTs | 9.5 |
| 459583 | AI907673 | | gb: IL-BT152-080399-004 BT152 Homo sapien | 9.3 |
| 452744 | AI267652 | Hs.30504 | Homo sapiens mRNA; cDNA DKFZp434E082 (fr | 9.2 |
| 432596 | AJ224741 | Hs.278461 | matrilin 3 | 9.1 |
| 400297 | AI127076 | Hs.334473 | hypothetical protein DKFZp564O1278 | 9.1 |
| 449448 | D60730 | Hs.57471 | ESTs | 9.1 |
| 423945 | AA410943 | | gb: zt32h03.r1 Soares ovary tumor NbHOT H | 9.1 |
| 406348 | | | Target Exon | 9.0 |
| 424735 | U31875 | Hs.272499 | short-chain alcohol dehydrogenase family | 9.0 |
| 453392 | U23752 | Hs.32964 | SRY (sex determining region Y)-box 11 | 9.0 |
| 433365 | AF026944 | Hs.293797 | ESTs | 8.8 |
| 405654 | NA | | C12001521: gi\|7513934\|pir\|\|T31081 cca3 pr | 8.8 |
| 418601 | AA279490 | Hs.86368 | calmegin | 8.8 |
| 451110 | AI955040 | Hs.265398 | ESTs, Weakly similar to transformation-r | 8.7 |
| 408771 | AW732573 | Hs.47584 | potassium voltage-gated channel, delayed | 8.5 |
| 409041 | AB033025 | Hs.50081 | KIAA1199 protein | 8.4 |
| 442353 | BE379594 | Hs.49136 | ESTs, Moderately similar to ALU7_HUMAN A | 8.3 |
| 451561 | N52812 | Hs.177403 | ESTs | 8.2 |
| 424001 | W67883 | Hs.137476 | paternally expressed 10 | 8.2 |
| 429859 | NM_007050 | Hs.225952 | protein tyrosine phosphatase, receptor t | 8.1 |
| 423887 | AL080207 | Hs.134585 | DKFZP434G232 protein | 8.1 |
| 405095 | NA | | Target Exon | 8.1 |
| 419296 | AA236115 | Hs.120785 | ESTs | 8.0 |
| 447164 | AF026941 | Hs.17518 | Homo sapiens cig5 mRNA, partial sequence | 8.0 |
| 413472 | BE242870 | Hs.75379 | solute carrier family 1 (glial high affi | 8.0 |
| 416747 | AW876523 | Hs.15929 | hypothetical protein FLJ12910 | 8.0 |
| 415385 | R17798 | Hs.7535 | COBW-like protein | 7.9 |
| 434424 | AI811202 | Hs.325335 | Homo sapiens cDNA: FLJ23523 fis, clone L | 7.9 |
| 420931 | AF044197 | Hs.100431 | small inducible cytokine B subfamily (Cy | 7.9 |
| 406687 | M31126 | Hs.272620 | matrix metalloproteinase 11 (MMP11; stro | 7.8 |
| 400285 | NA | | Eos Control | 7.7 |
| 437207 | T27503 | Hs.15929 | hypothetical protein FLJ12910 | 7.6 |
| 427119 | AW880562 | Hs.114574 | ESTs | 7.5 |
| 429534 | AW976987 | Hs.163327 | ESTs, Weakly similar to 2109260A B cell | 7.5 |
| 433426 | H69125 | Hs.133525 | ESTs | 7.5 |
| 411078 | AI222020 | Hs.182364 | CocoaCrisp | 7.4 |
| 426214 | H59846 | Hs.128355 | ESTs, Moderately similar to ALU7_HUMAN A | 7.4 |
| 447475 | AI380797 | Hs.158992 | ESTs | 7.3 |
| 415263 | AA948033 | Hs.130853 | ESTs | 7.2 |
| 439569 | AW602166 | Hs.222399 | CEGP1 protein | 7.2 |
| 414142 | AW368397 | Hs.150042 | Homo sapiens cDNA FLJ14438 fis, clone HE | 7.1 |
| 426261 | AW242243 | Hs.168670 | peroxisomal famesylated protein | 7.0 |
| 444783 | AK001468 | Hs.62180 | anillin (Drosophila Scraps homolog), act | 6.9 |
| 445885 | AI734009 | Hs.127699 | KIAA1603 protein | 6.9 |
| 429432 | AI678059 | Hs.202676 | synaptonemal complex protein 2 | 6.9 |
| 410781 | AI375672 | Hs.165028 | ESTs | 6.9 |
| 443788 | AI732643 | Hs.144151 | ESTs | 6.9 |
| 421373 | AA808229 | Hs.167771 | ESTs | 6.8 |
| 451398 | AI793124 | Hs.144479 | ESTs | 6.8 |
| 404253 | | | NM_021058*: Homo sapiens H2B histone fami | 6.8 |
| 441098 | AI015591 | Hs.131004 | ESTs, Weakly similar to T17227 hypotheti | 6.7 |
| 426215 | AW963419 | Hs.155223 | stanniocalcin 2 | 6.6 |
| 428227 | AA321649 | Hs.2248 | small inducible cytokine subfamily B (CX | 6.6 |
| 422956 | BE545072 | Hs.122579 | hypothetical protein FLJ10461 | 6.6 |
| 411111 | AW818127 | | gb: CM1-ST0277-061299-059-b07 ST0277 Homo | 6.6 |
| 434988 | AI418055 | Hs.161160 | ESTs | 6.6 |
| 442580 | AI733682 | Hs.130239 | ESTs | 6.6 |
| 449611 | AI970394 | Hs.197075 | ESTs | 6.6 |
| 408000 | L11690 | Hs.620 | bullous pemphigoid antigen 1 (230/240 kD) | 6.5 |
| 420757 | X78592 | Hs.99915 | androgen receptor (dihydrotestosterone r | 6.5 |
| 431089 | BE041395 | Hs.283676 | ESTs, Weakly similar to unknown protein | 6.5 |
| 400301 | X03635 | Hs.1657 | estrogen receptor 1 | 6.5 |
| 427356 | AW023482 | Hs.97849 | ESTs | 6.5 |
| 425704 | U79293 | Hs.159264 | Human clone 23948 mRNA sequence | 6.4 |
| 441134 | W29092 | Hs.7678 | cellular retinoic acid-binding protein 1 | 6.4 |
| 424902 | NM_003866 | Hs.153687 | inositol polyphosphate-4-phosphatase, ty | 6.4 |
| 448693 | AW004854 | Hs.228320 | hypothetical protein FLJ23537 | 6.4 |
| 431448 | AL137517 | Hs.334473 | hypothetical protein DKFZp564O1278 | 6.2 |
| 444342 | NM_014398 | Hs.10887 | similar to lysosome-associated membrane | 6.1 |
| 422168 | AA586894 | Hs.112408 | S100 calcium-binding protein A7 (psorias | 6.1 |
| 453331 | AI240665 | Hs.8895 | ESTs | 6.1 |
| 418007 | M13509 | Hs.83169 | matrix metalloproteinase 1 (MMP1; inters | 6.0 |
| 441233 | AA972965 | Hs.135568 | ESTs | 6.0 |
| 418092 | R45154 | Hs.106604 | ESTs | 6.0 |
| 430044 | AA464510 | Hs.152812 | ESTs | 5.9 |
| 432837 | AA310693 | Hs.87329 | HSPC072 protein | 5.9 |
| 433285 | AW975944 | Hs.237396 | ESTs | 5.9 |
| 450701 | H39960 | Hs.288467 | Homo sapiens cDNA FLJ12280 fis, clone MA | 5.9 |
| 425707 | AF115402 | Hs.11713 | E74-like factor 5 (ets domain transcript | 5.9 |
| 410785 | AW803341 | | gb: IL2-UM0079-090300-050-D03 UM0079 Homo | 5.9 |
| 425398 | AL049689 | Hs.156369 | hypothetical protein similar to tenascin | 5.9 |
| 414812 | X72755 | Hs.77367 | monokine induced by gamma interferon | 5.8 |
| 459371 | R20991 | | gb: yg06h01.r1 Soares infant brain 1NIB H | 5.8 |
| 411284 | N28519 | Hs.135191 | ESTs, Weakly similar to unnamed protein | 5.8 |
| 453511 | AL031224 | Hs.33102 | transcription factor AP-2 beta (activati | 5.8 |
| 451807 | W52854 | Hs.27099 | hypothetical protein FLJ23293 similar to | 5.7 |
| 430510 | AW162916 | Hs.241576 | hypothetical protein PRO2577 | 5.7 |
| 415539 | AI733881 | Hs.72472 | BMP-R1B | 5.6 |
| 438199 | AW016531 | Hs.122147 | ESTs | 5.6 |
| 417866 | AW067903 | Hs.82772 | collagen, type XI, alpha 1 | 5.5 |
| 430019 | AA463893 | Hs.220933 | ESTs | 5.5 |
| 439809 | R41396 | Hs.101774 | hypothetical protein FLJ23045 | 5.5 |
| 423811 | AW299598 | Hs.50895 | homeo box C4 | 5.4 |
| 434539 | AW748078 | Hs.214410 | ESTs, Weakly similar to MUC2_HUMAN MUCIN | 5.4 |
| 439138 | AI742605 | Hs.193696 | ESTs | 5.4 |
| 453931 | AL121278 | Hs.25144 | ESTs | 5.4 |
| 444078 | BE246919 | Hs.10290 | U5 snRNP-specific 40 kDa protein (hPrp8- | 5.4 |
| 447102 | BE167434 | Hs.98471 | ESTs, Weakly similar to T18712 hypotheti | 5.4 |
| 451621 | AI879148 | Hs.26770 | fatty acid binding protein 7, brain | 5.4 |
| 425236 | AW067800 | Hs.155223 | stanniocalcin 2 | 5.3 |
| 421464 | AA291553 | Hs.190086 | ESTs | 5.3 |
| 450736 | AW970060 | | gb: EST382140 MAGE resequences, MAGK Homo | 5.3 |
| 428085 | AA421081 | Hs.12388 | ESTs | 5.3 |
| 452838 | U65011 | Hs.30743 | preferentially expressed antigen in mela | 5.3 |
| 445424 | AB028945 | Hs.12696 | cortactin SH3 domain-binding protein | 5.3 |
| 456938 | X52509 | Hs.161640 | tyrosine aminotransferase | 5.3 |
| 422867 | L32137 | Hs.1584 | cartilage oligomeric matrix protein (COM | 5.2 |
| 438167 | R28363 | Hs.24286 | ESTs | 5.2 |
| 433330 | AW207084 | Hs.132816 | hypothetical protein MGC14801 | 5.2 |
| 449765 | N92293 | Hs.206832 | ESTs, Moderately similar to ALU8_HUMAN A | 5.2 |
| 416276 | U41060 | Hs.79136 | LIV-1 protein, estrogen regulated | 5.2 |
| 400300 | X03363 | | HER2 receptor tyrosine kinase (c-erb-b2, | 5.2 |
| 418004 | U37519 | Hs.87539 | aldehyde dehydrogenase 3 family, member | 5.2 |
| 439840 | AW449211 | Hs.105445 | GDNF family receptor alpha 1 | 5.2 |
| 428771 | AB028992 | Hs.193143 | KIAA1069 protein | 5.2 |
| 455047 | AW852530 | | gb: PM1-CT0243-071099-001-g06 CT0243 Homo | 5.2 |
| 419169 | AW851980 | Hs.262346 | ESTs, Weakly similar to S72482 hypotheti | 5.2 |
| 453197 | AI916269 | Hs.109057 | ESTs, Weakly similar to ALU5_HUMAN ALU S | 5.1 |
| 400298 | AA032279 | Hs.61635 | six transmembrane epithelial antigen of | 5.1 |
| 431023 | AI283133 | Hs.297420 | ESTs | 5.1 |
| 427666 | AI791495 | Hs.180142 | calmodulin-like skin protein | 5.1 |
| 427718 | AI798680 | Hs.25933 | ESTs | 5.1 |
| 434531 | AA642007 | Hs.116369 | ESTs | 5.1 |
| 429220 | AW207206 | Hs.136319 | ESTs | 5.1 |
| 405494 | NA | | C2001837*: gi\|12697903\|dbj\|BAB21770.1\|(A | 5.1 |
| 452930 | AW195285 | Hs.194097 | ESTs, Weakly similar to I38022 hypotheti | 5.1 |
| 444910 | AI201849 | | gb: qs76g04.x1 NCI_CGAP_Pr28 Homo sapiens | 5.1 |
| 453310 | X70697 | Hs.553 | solute carrier family 6 (neurotransmitte | 5.0 |
| 444381 | BE387335 | Hs.283713 | ESTs, Weakly similar to S64054 hypotheti | 5.0 |
| 450603 | R43646 | Hs.12422 | ESTs | 5.0 |
| 416575 | W02414 | Hs.38383 | ESTs | 5.0 |
| 438504 | AW665281 | Hs.224625 | ESTs | 5.0 |
| 416209 | AA236776 | Hs.79078 | MAD2 (mitotic arrest deficient, yeast, h | 5.0 |
| 428804 | AK000713 | Hs.193736 | hypothetical protein FLJ20706 | 5.0 |
| 420077 | AW512260 | Hs.87767 | ESTs | 4.9 |
| 450480 | X82125 | Hs.25040 | zinc finger protein 239 | 4.9 |
| 437637 | AJ003029 | Hs.65792 | syntrophin, gamma 2 | 4.9 |
| 431808 | M30703 | Hs.270833 | amphiregulin (schwannoma-derived growth | 4.9 |
| 418836 | AI655499 | Hs.161712 | ESTs | 4.8 |
| 442441 | AI820662 | Hs.129598 | ESTs | 4.8 |
| 435635 | AF220050 | Hs.181385 | uncharacterized hematopoietic stem/proge | 4.8 |
| 400286 | NA | | C16000922: gi\|7499103\|pir\|\|T20903 hypothe | 4.8 |
| 407506 | U71600 | | gb: Human zinc finger protein zfp31 (zf31 | 4.8 |
| 420026 | AI831190 | Hs.166676 | ESTs | 4.8 |
| 441377 | BE218239 | Hs.202656 | ESTs | 4.8 |
| 457726 | AI217477 | Hs.194591 | ESTs | 4.8 |
| 412785 | AW997556 | Hs.78521 | KIAA1717 protein | 4.8 |
| 428368 | BE440042 | Hs.83326 | matrix metalloproteinase 3 (stromelysin | 4.7 |
| 436026 | AI349764 | Hs.217081 | ESTs | 4.7 |
| 409110 | AA191493 | Hs.48778 | niban protein | 4.7 |
| 400284 | NA | | estrogen receptor 1 | 4.7 |
| 410102 | AW248508 | Hs.279727 | Homo sapiens cDNA FLJ14035 fis, clone HE | 4.7 |
| 407819 | R42185 | Hs.274803 | ESTs | 4.7 |
| 430486 | BE062109 | Hs.241551 | chloride channel, calcium activated, fam | 4.7 |
| 422896 | AW961489 | Hs.154116 | ESTs | 4.7 |
| 453616 | NM_003462 | Hs.33846 | dynein, axonemal, light intermediate pol | 4.7 |
| 427427 | AF077345 | Hs.177936 | ESTs | 4.6 |
| 421751 | AW813731 | Hs.159153 | ESTs, Moderately similar to S65657 alpha | 4.6 |
| 454074 | R63503 | Hs.28419 | ESTs | 4.6 |
| 405718 | | | C4000799*: gi\|6330365\|dbj\|BAA86508.1\|(AB | 4.6 |
| 444649 | AW207523 | Hs.197628 | ESTs | 4.6 |
| 429431 | Z40313 | Hs.106330 | Homo sapiens clone IMAGE: 23371, mRNA seq | 4.6 |
| 427811 | M81057 | Hs.180884 | carboxypeptidase B1 (tissue) | 4.6 |
| 447342 | AI199268 | Hs.19322 | Homo sapiens , Similar to RIKEN cDNA 2010 | 4.6 |
| 430345 | AK000282 | Hs.239681 | hypothetical protein FLJ20275 | 4.6 |
| 454307 | AW855717 | | gb: RC1-CT0279-081299-013-b01 CT0279 Homo | 4.6 |
| 400303 | AA242758 | Hs.79136 | LIV-1 protein, estrogen regulated | 4.6 |
| 438180 | AA808189 | Hs.272151 | ESTs | 4.6 |
| 451340 | AW936273 | | gb: QV0-DT0020-090200-107-g07 DT0020 Homo | 4.6 |
| 458711 | AL036877 | Hs.282878 | ESTs | 4.6 |
| 457430 | AA514660 | Hs.128443 | ESTs | 4.6 |
| 416030 | H15261 | Hs.21948 | ESTs | 4.6 |
| 447233 | AW246333 | Hs.17901 | Homo sapiens , clone IMAGE: 3937015, mRNA, | 4.6 |
| 445537 | AJ245671 | Hs.12844 | EGF-like-domain, multiple 6 (EGFL6) | 4.5 |
| 424590 | AW966399 | Hs.46821 | hypothetical protein FLJ20086 | 4.5 |
| 432374 | W68815 | Hs.301885 | Homo sapiens cDNA FLJ11346 fis, clone PL | 4.5 |
| 423833 | AW503329 | | gb: UI-HF-BN0-akx-e-02-0-UI.r1 NIH_MGC_50 | 4.5 |
| 406747 | AI925153 | Hs.217493 | annexin A2 | 4.5 |
| 412102 | H56435 | | gb: yq98e09.r1 Soares fetal liver spleen | 4.5 |
| 431716 | D89053 | Hs.268012 | fatty-acid-Coenzyme A ligase, long-chain | 4.5 |
| 411050 | AW814902 | | gb: MR1-ST0206-120400-022-f08 ST0206 Homo | 4.5 |
| 401418 | NA | | C14000338*: gi\|7459502\|pir\|\|S74665 outer | 4.5 |
| 436194 | AK001074 | Hs.333435 | Homo sapiens cDNA FLJ10212 fis, clone HE | 4.5 |
| 436211 | AK001581 | Hs.334828 | hypothetical protein FLJ10719; KIAA1794 | 4.4 |
| 414080 | AA135257 | Hs.47783 | B aggressive lymphoma gene | 4.4 |
| 424115 | AA335497 | Hs.293965 | ESTs, Weakly similar to I38022 hypotheti | 4.4 |
| 415786 | AW419196 | Hs.257924 | hypothetical protein FLJ13782 | 4.4 |
| 442117 | AW664964 | Hs.128899 | ESTs | 4.4 |
| 452784 | BE463857 | Hs.151258 | hypothetical protein FLJ21062 | 4.4 |
| 432731 | R31178 | Hs.287820 | fibronectin 1 | 4.4 |
| 410534 | AW905138 | | gb: QV0-NN1071-280400-207-g07 NN1071 Homo | 4.4 |
| 405196 | NA | | C2000662*: gi\|7512792\|pir\|\|T12482 hypothe | 4.4 |
| 430217 | N47863 | Hs.336901 | ribosomal protein S24 | 4.4 |
| 401793 | | | C17001545: gi\|5360127\|gb\|AAD42882.1\|AF155 | 4.4 |
| 415747 | AA381209 | | gb: EST94257 Activated T-cells I Homo sap | 4.4 |
| 423679 | AB007975 | Hs.131454 | KIAA0506 protein | 4.4 |
| 400238 | NA | | C19000274*: gi\|12741327\|ref\|XP_008833.2\| | 4.4 |
| 425627 | AF019612 | Hs.297007 | membrane-bound transcription factor prot | 4.4 |
| 400608 | | | C10001899: gi\|7508633\|pir\|\|T25392 hypothe | 4.4 |
| 458634 | AV657310 | Hs.282898 | ESTs | 4.3 |
| 407771 | AL138272 | Hs.62713 | ESTs | 4.3 |
| 405906 | NA | | Target Exon | 4.3 |
| 405925 | NA | | Target Exon | 4.3 |
| 439382 | BE247684 | Hs.103070 | ESTs | 4.3 |
| 445263 | H57646 | Hs.42586 | KIAA1560 protein | 4.3 |
| 407162 | N63855 | Hs.142634 | zinc finger protein | 4.3 |
| 419536 | AA603305 | | gb: np12d11.s1 NCI_CGAP_Pr3 Homo sapiens | 4.3 |
| 454359 | N71277 | | gb: za36e03.s1 Soares fetal liver spleen | 4.3 |
| 411558 | AA102670 | Hs.70725 | gamma-aminobutyric acid (GABA) A recepto | 4.2 |
| 450715 | AI266484 | Hs.31570 | ESTs, Weakly similar to KIAA1324 protein | 4.2 |
| 421451 | AA291377 | Hs.50831 | ESTs | 4.2 |
| 452864 | AA033714 | Hs.287629 | hypothetical protein FLJ14260 | 4.2 |
| 409757 | NM_001898 | Hs.123114 | cystatin SN | 4.2 |
| 413043 | BE158766 | | gb: IL2-HT0397-071299-024-F02 HT0397 Homo | 4.2 |
| 413499 | BE144884 | | gb: CM0-HT0182-041099-065-e11 HT0182 Homo | 4.2 |
| 444619 | BE538082 | Hs.8172 | ESTs, Moderately similar to A46010 X-lin | 4.2 |
| 408380 | AF123050 | Hs.44532 | diubiquitin | 4.2 |
| 406992 | S82472 | | gb: beta-pol = DNA polymerase beta {exon a | 4.2 |
| 404285 | NA | | C6001909: gi\|704441\|dbj\|BAA18909.1\| (D298 | 4.2 |
| 425247 | NM_005940 | Hs.155324 | matrix metalloproteinase 11 (MMP11; stro | 4.2 |
| 428046 | AW812795 | Hs.155381 | ESTs, Moderately similar to I38022 hypot | 4.2 |
| 446163 | AA026880 | Hs.25252 | prolactin receptor | 4.2 |
| 421147 | AW592167 | Hs.293299 | ESTs | 4.2 |
| 426451 | AI908165 | Hs.169946 | GATA-binding protein 3 (T-cell receptor | 4.2 |
| 415227 | AW821113 | Hs.72402 | ESTs | 4.2 |
| 452176 | AA024538 | Hs.282990 | Human DNA sequence from clone RP1-28H20 | 4.2 |
| 452862 | AW378065 | Hs.8687 | ESTs | 4.2 |
| 443646 | AI085198 | Hs.164226 | ESTs | 4.2 |
| 425523 | AB007948 | Hs.158244 | KIAA0479 protein | 4.1 |
| 424687 | J05070 | Hs.151738 | matrix metalloproteinase 9 (gelatinase B | 4.1 |
| 430009 | AA894564 | Hs.22242 | ESTs | 4.1 |
| 434469 | AA634806 | | gb: ab28c02.r1 Stratagene lung (937210) H | 4.1 |
| 451381 | BE241831 | Hs.172330 | hypothetical protein MGC2705 | 4.1 |
| 450229 | R18717 | Hs.8929 | hypothetical protein FLJ11362 | 4.1 |
| 455700 | BE068115 | | gb: CM1-BT0368-061299-060-g07 BT0368 Homo | 4.1 |
| 431924 | AK000850 | Hs.272203 | Homo sapiens cDNA FLJ20843 fis, clone AD | 4.1 |
| 438885 | AI886558 | Hs.184987 | ESTs | 4.1 |
| 401451 | | | NM_004496*: Homo sapiens hepatocyte nucle | 4.1 |
| 431676 | AI685464 | | gb: tt88f04.x1 NCI_CGAP_Pr28 Homo sapiens | 4.1 |
| 409092 | AI735283 | Hs.172608 | ESTs | 4.1 |
| 429270 | W60379 | Hs.57773 | ESTs | 4.1 |
| 443903 | AI220547 | Hs.135223 | ESTs | 4.1 |
| 427122 | AW057736 | Hs.323910 | HER2 receptor tyrosine kinase (c-erb-b2, | 4.1 |
| 410275 | U85658 | Hs.61796 | transcription factor AP-2 gamma (activat | 4.1 |
| 432912 | BE007371 | Hs.200313 | ESTs | 4.1 |
| 403585 | | | Target Exon | 4.1 |
| 438295 | AI394151 | Hs.37932 | ESTs | 4.1 |
| 420380 | AA640891 | Hs.102406 | ESTs | 4.1 |
| 431118 | BE264901 | Hs.250502 | carbonic anhydrase VIII | 4.1 |
| 416182 | NM_004354 | Hs.79069 | cyclin G2 | 4.1 |
| 418994 | AA296520 | Hs.89546 | selectin E (endothelial adhesion molecul | 4.1 |
| 400555 | | | Target Exon | 4.1 |
| 410079 | U94362 | Hs.58589 | glycogenin 2 | 4.0 |
| 427674 | NM_003528 | Hs.2178 | H2B histone family, member Q | 4.0 |
| 427131 | AA448460 | Hs.112017 | GE36 gene | 4.0 |
| 439759 | AL359055 | Hs.67709 | Homo sapiens mRNA full length insert cDN | 4.0 |
| 429353 | AL117406 | Hs.200102 | ATP-binding cassette transporter MRP8 | 4.0 |
| 421296 | NM_002666 | Hs.103253 | perilipin | 4.0 |
| 418819 | AA228776 | Hs.191721 | ESTs | 4.0 |
| 424188 | AW954552 | Hs.142634 | zinc finger protein | 4.0 |
| 455431 | AW938484 | | gb: CM0-DT0057-290200-253-d06 DT0057 Homo | 4.0 |
| 404142 | NA | | Target Exon | 4.0 |
| 441143 | AI027604 | Hs.159650 | ESTs | 4.0 |
| 444540 | AI693927 | Hs.265165 | ESTs | 4.0 |
| 415579 | AA165232 | Hs.222069 | ESTs | 4.0 |
| 452891 | N75582 | Hs.212875 | ESTs, Weakly similar to DYH9_HUMAN CILIA | 4.0 |
| 414605 | BE390440 | | gb: 601283601F1 NIH_MGC_44 Homo sapiens c | 4.0 |
| 452281 | T93500 | Hs.28792 | Homo sapiens cDNA FLJ11041 fis, clone PL | 4.0 |
| 417801 | AA417383 | Hs.82582 | integrin, beta-like 1 (with EGF-like rep | 4.0 |
| 446232 | AI281848 | Hs.194691 | retinoic acid induced 3 | 4.0 |
| 447377 | X77343 | Hs.334334 | transcription factor AP-2 alpha (activat | 4.0 |
| 437854 | AL119723 | | gb: DKFZp761A2124_r1 761 (synonym: hamy2) | 4.0 |
| 446140 | AA356170 | Hs.26750 | hypothetical protein FLJ21908 | 4.0 |
| 452240 | AI591147 | Hs.61232 | ESTs | 4.0 |
| 459574 | AI741122 | Hs.101810 | Homo sapiens cDNA FLJ14232 fis, clone NT | 4.0 |
| 458673 | N99626 | | gb: za39d11.r1 Soares fetal liver spleen | 4.0 |
| 444858 | AI199738 | Hs.208275 | ESTs, Weakly similar to ALUA_HUMAN !!!! | 4.0 |
| 452166 | AI948607 | Hs.264680 | ESTs | 4.0 |
| 452681 | AF153330 | Hs.30246 | solute carrier family 19 (thiamine trans | 3.9 |
| 450192 | AA263143 | Hs.24596 | RAD51-interacting protein | 3.9 |
| 406554 | NA | | Target Exon | 3.9 |
| 416259 | AA573006 | Hs.19173 | ESTs | 3.9 |
| 445813 | Z42023 | Hs.106576 | alanine-glyoxylate aminotransferase 2-li | 3.9 |
| 451024 | AA442176 | | gb: zw63b08.r1 Soares_total_fetus_Nb2HF8_{—} | 3.9 |
| 413930 | M86153 | Hs.75618 | RAB11A, member RAS oncogene family | 3.9 |
| 401781 | | | Target Exon | 3.9 |
| 415296 | F05086 | Hs.328142 | ESTs | 3.9 |
| 452564 | AA026777 | | gb: ze93c11.r1 Soares_fetal_heart_NbHH19W | 3.9 |
| 442500 | AI819068 | Hs.209122 | ESTs | 3.9 |
| 419759 | Z21336 | Hs.135411 | actin related protein | 3.9 |
| 424638 | AI472106 | Hs.49303 | Homo sapiens cDNA FLJ11663 fis, clone HE | 3.9 |
| 439699 | AF086534 | Hs.187561 | ESTs, Moderately similar to ALU1_HUMAN A | 3.9 |
| 428042 | AA419529 | Hs.76391 | myxovirus (influenza) resistance 1, homo | 3.9 |
| 452501 | AB037791 | Hs.29716 | hypothetical protein FLJ10980 | 3.9 |
| 453049 | BE537217 | Hs.30343 | ESTs | 3.9 |
| 443213 | BE568414 | Hs.145497 | Homo sapiens cDNA: FLJ22097 fis, clone H | 3.9 |
| 443489 | AI073512 | Hs.133916 | ESTs | 3.9 |
| 455092 | BE152428 | | gb: CM0-HT0323-151299-126-b04 HT0323 Homo | 3.9 |
| 401785 | | | NM_002275*: Homo sapiens keratin 15 (KRT1 | 3.9 |
| 426427 | M86699 | Hs.169840 | TTK protein kinase | 3.9 |
| 446009 | AI989885 | Hs.231926 | ESTs | 3.9 |
| 436033 | H75391 | Hs.255748 | ESTs | 3.9 |
| 451067 | BE172186 | | gb: MR0-HT0559-110300-005-h11 HT0559 Homo | 3.8 |
| 419348 | AA236645 | Hs.98274 | ESTs | 3.8 |
| 444635 | AI184268 | Hs.339665 | ESTs | 3.8 |
| 412140 | AA219691 | Hs.73625 | RAB6 interacting, kinesin-like (rabkines | 3.8 |
| 403593 | NA | | Target Exon | 3.8 |
| 442323 | AW016669 | Hs.29190 | ESTs | 3.8 |
| 419854 | AW664873 | Hs.87836 | Homo sapiens PAC clone RP5-1087M19 from | 3.8 |
| 433871 | W02410 | Hs.205555 | ESTs | 3.8 |
| 445253 | AI217928 | Hs.144762 | ESTs | 3.8 |
| 409542 | AA503020 | Hs.36563 | hypothetical protein FLJ22418 | 3.8 |
| 443162 | T49951 | Hs.9029 | DKFZP434G032 protein | 3.8 |
| 458194 | AW383618 | Hs.265459 | ESTs, Moderately similar to ALU2_HUMAN A | 3.8 |
| 422475 | AL359938 | Hs.117313 | Meis (mouse) homolog 3 | 3.8 |
| 440705 | AA904244 | Hs.153205 | ESTs | 3.8 |
| 447290 | AI476732 | Hs.263912 | ESTs | 3.8 |
| 403426 | | | Target Exon | 3.8 |
| 427821 | AA470158 | Hs.98202 | ESTs | 3.8 |
| 454288 | BE222648 | Hs.279458 | ESTs, Highly similar to c380A1.1b [H. sap | 3.8 |
| 443801 | AW206942 | Hs.253594 | ESTs | 3.8 |
| 410658 | AW105231 | Hs.192035 | ESTs | 3.8 |
| 410672 | AW794600 | | gb: RC6-UM0014-170300-022-C05 UM0014 Homo | 3.8 |
| 428579 | NM_005756 | Hs.184942 | G protein-coupled receptor 64 | 3.8 |
| 445495 | BE622641 | Hs.38489 | ESTs, Weakly similar to I38022 hypotheti | 3.8 |
| 447995 | AI742618 | Hs.181733 | ESTs, Weakly similar to nitrilase homolo | 3.7 |
| 401747 | | | Homo sapiens keratin 17 (KRT17) | 3.7 |
| 420633 | NM_014581 | Hs.274480 | odorant-binding protein 2A | 3.7 |
| 423545 | AP000692 | Hs.129781 | chromosome 21 open reading frame 5 | 3.7 |
| 433138 | AB029496 | Hs.59729 | semaphorin sem2 | 3.7 |
| 434715 | BE005346 | Hs.116410 | ESTs | 3.7 |
| 428664 | AK001666 | Hs.189095 | similar to SALL1 (sal (Drosophila)-like | 3.7 |
| 450951 | AA018534 | Hs.103334 | ESTs | 3.7 |
| 402696 | NA | | C3002523: gi\|6686211\|sp\|Q27533\|YH2M_CAEEL | 3.7 |
| 446868 | AV660737 | Hs.135100 | ESTs | 3.7 |
| 458154 | AW816379 | Hs.335018 | ESTs | 3.7 |
| 422026 | U80736 | Hs.110826 | trinucleotide repeat containing 9 | 3.7 |
| 419440 | AB020689 | Hs.90419 | KIAA0882 protein | 3.7 |
| 421524 | AA312082 | Hs.105445 | GDNF family receptor alpha 1 | 3.7 |
| 417283 | N62840 | Hs.48648 | ESTs | 3.7 |
| 401508 | NA | | NM_024817: Homo sapiens hypothetical prot | 3.7 |
| 410303 | AA324597 | Hs.21851 | Homo sapiens cDNA FLJ12900 fis, clone NT | 3.7 |
| 420362 | U79734 | Hs.97206 | huntingtin interacting protein 1 | 3.7 |
| 433384 | AI021992 | Hs.124244 | ESTs | 3.7 |
| 434302 | AA629065 | Hs.116301 | ESTs | 3.7 |
| 443938 | R55373 | Hs.20864 | ESTs | 3.7 |
| 448420 | BE623004 | | gb: 601441282F1 NIH_MGC_72 Homo sapiens c | 3.7 |
| 458712 | AI347502 | Hs.107872 | hypothetical protein FLJ20761 | 3.7 |
| 433404 | T32982 | Hs.102720 | ESTs | 3.7 |
| 405232 | | | NM_015832: Homo sapiens methyl-CpG bindin | 3.7 |
| 430491 | AL109791 | Hs.241559 | Homo sapiens mRNA full length insert cDN | 3.7 |
| 455609 | BE011668 | | gb: CM3-BN0223-100500-177-a04 BN0223 Homo | 3.7 |
| 450164 | AI239923 | Hs.30098 | ESTs | 3.7 |
| 453948 | AI970797 | Hs.64859 | ESTs | 3.7 |
| 436061 | AI248584 | Hs.190745 | Homo sapiens cDNA: FLJ21326 fis, clone C | 3.7 |
| 401049 | NA | | Target Exon | 3.6 |
| 418867 | D31771 | Hs.89404 | msh (Drosophila) homeo box homolog 2 | 3.6 |
| 420179 | N74530 | Hs.21168 | ESTs | 3.6 |
| 458663 | AV658444 | Hs.280776 | tankyrase, TRF1-interacting ankyrin-rela | 3.6 |
| 437259 | AI377755 | Hs.120695 | ESTs | 3.6 |
| 428309 | M97815 | Hs.183650 | cellular retinoic acid-binding protein 2 | 3.6 |
| 450522 | AI698839 | | gb: wd31f02.x1 Soares_NFL_T_GBC_S1 Homo s | 3.6 |
| 451952 | AL120173 | Hs.301663 | ESTs | 3.6 |
| 412209 | AW901456 | | gb: RC0-NN1012-270300-031-c07 NN1012 Homo | 3.6 |
| 425201 | AA352111 | | gb: EST60061 Activated T-cells XX Homo sa | 3.6 |
| 443830 | AI142095 | Hs.143273 | ESTs | 3.6 |
| 439255 | BE164500 | | gb: RC4-HT0469-230300-014-e10 HT0469 Homo | 3.6 |
| 414869 | AA157291 | Hs.21479 | ubinuclein 1 | 3.6 |
| 409064 | AA062954 | Hs.141883 | ESTs | 3.6 |
| 407721 | Y12735 | Hs.38018 | dual-specificity tyrosine-(Y)-phosphoryl | 3.6 |
| 445135 | AK000054 | Hs.12347 | hypothetical protein FLJ20047 | 3.6 |
| 404091 | NA | | Target Exon | 3.6 |
| 409731 | AA125985 | Hs.56145 | thymosin, beta, identified in neuroblast | 3.6 |
| 405153 | | | Target Exon | 3.6 |
| 423248 | AA380177 | Hs.125845 | ribulose-5-phosphate-3-epimerase | 3.6 |
| 403639 | NA | | ENSP00000233023*: CDNA FLJ12662 fis, clon | 3.6 |
| 404360 | | | C7001385: gi\|12082809\|gb\|AAG48618.1\|AF315 | 3.6 |
| 422352 | AA766296 | Hs.99200 | ESTs | 3.6 |
| 423338 | AB007961 | Hs.127338 | KIAA0492 protein | 3.6 |
| 424202 | BE350295 | Hs.15032 | RAN binding protein 17 | 3.6 |
| 431750 | AA514986 | Hs.283705 | ESTs | 3.6 |
| 439907 | AA853978 | Hs.124577 | ESTs | 3.6 |
| 453596 | AA441838 | Hs.62905 | hypothetical protein FLJ14834 | 3.6 |
| 406446 | NA | | Target Exon | 3.6 |
| 418454 | AA315308 | Hs.195870 | hypothetical protein FLJ14991 | 3.6 |
| 434360 | AW015415 | Hs.127780 | ESTs | 3.6 |
| 409079 | W87707 | Hs.82065 | interleukin 6 signal transducer (gp130, | 3.6 |
| 440132 | AI697121 | Hs.202466 | ESTs, Weakly similar to S65824 reverse t | 3.6 |
| 448706 | AW291095 | Hs.21814 | interleukin 20 receptor, alpha | 3.6 |
| 440671 | AW297920 | Hs.130054 | ESTs | 3.5 |
| 407647 | AW860158 | | gb: RC0-CT0379-290100-032-b04 CT0379 Homo | 3.5 |
| 459023 | AW968226 | Hs.60798 | ESTs | 3.5 |
| 402820 | | | NM_017646*: Homo sapiens tRNA isopentenyl | 3.5 |
| 417009 | AA191719 | Hs.314714 | ESTs | 3.5 |
| 447048 | AW393080 | Hs.228320 | hypothetical protein FLJ23537 | 3.5 |
| 449978 | AI806335 | Hs.200829 | ESTs, Weakly similar to T30171 ninein - | 3.5 |
| 428062 | AA420683 | Hs.98321 | hypothetical protein FLJ14103 | 3.5 |
| 452909 | NM_015368 | Hs.30985 | pannexin 1 | 3.5 |
| 400610 | NA | | Target Exon | 3.5 |
| 417843 | W07361 | Hs.22545 | Homo sapiens cDNA FLJ12935 fis, clone NT | 3.5 |
| 419335 | AW960146 | Hs.284137 | hypothetical protein FLJ12888 | 3.5 |
| 451592 | AI805416 | Hs.213897 | ESTs | 3.5 |
| 443270 | NM_004272 | Hs.337737 | Homer, neuronal immediate early gene, 1B | 3.5 |
| 423948 | AW392342 | Hs.283077 | centrosomal P4.1-associated protein; unc | 3.5 |
| 449424 | AW448937 | Hs.197030 | ESTs | 3.5 |
| 423641 | AW753967 | | gb: RC2-CT0304-080100-011-h12 CT0304 Homo | 3.5 |
| 416806 | NM_000288 | Hs.79993 | peroxisomal biogenesis factor 7 | 3.5 |
| 422060 | R20893 | Hs.325823 | ESTs, Moderately similar to ALU5_HUMAN A | 3.5 |
| 433104 | AL043002 | Hs.128246 | ESTs, Moderately similar to unnamed prot | 3.5 |
| 415778 | H84847 | Hs.49391 | hypothetical protein LOC54149 | 3.5 |
| 413054 | AW316843 | Hs.66309 | hypothetical protein MGC11061 | 3.5 |
| 416636 | N32536 | Hs.42645 | solute carrier family 16 (monocarboxylic | 3.5 |
| 424639 | AI917494 | Hs.9812 | Homo sapiens cDNA FLJ14388 fis, clone HE | 3.5 |
| 424827 | AI057094 | Hs.96867 | Homo sapiens cDNA: FLJ23155 fis, clone L | 3.5 |
| 437782 | AI370876 | Hs.79090 | exportin 1 (CRM1, yeast, homolog) | 3.5 |
| 411514 | AW850178 | | gb: IL3-CT0219-271099-022-H12 CT0219 Homo | 3.5 |
| 413783 | AA314337 | Hs.301547 | ribosomal protein S7 | 3.5 |
| 421106 | AA877124 | Hs.172844 | ESTs | 3.5 |
| 431291 | N25521 | Hs.25275 | Kruppel-type zinc finger protein | 3.5 |
| 440623 | AI935016 | Hs.216639 | ESTs | 3.5 |
| 455838 | BE145808 | | gb: MR0-HT0208-101299-103-f11 HT0208 Homo | 3.5 |
| 458771 | AW295151 | Hs.163612 | ESTs | 3.5 |
| 442942 | AW167087 | Hs.131562 | ESTs | 3.5 |
| 436550 | Z50158 | Hs.270235 | ESTs, Weakly similar to MMHUB1 laminin b | 3.5 |
| 418849 | AW474547 | Hs.53565 | Homo sapiens PIG-M mRNA for mannosyltran | 3.5 |
| 424420 | BE614743 | Hs.146688 | prostaglandin E synthase | 3.5 |
| 430916 | AW505021 | Hs.88414 | BTB and CNC homology 1, basic leucine zi | 3.5 |
| 432030 | AI908400 | Hs.143789 | ESTs | 3.5 |
| 439405 | AF086224 | Hs.55238 | ESTs | 3.5 |
| 405917 | NA | | C17000675: gi\|7290703\|gb\|AAF46150.1\| (AE0 | 3.5 |
| 452727 | AW993582 | Hs.176220 | ESTs | 3.5 |
| 426320 | W47595 | Hs.169300 | transforming growth factor, beta 2 | 3.4 |
| 421070 | AA283185 | Hs.19327 | ESTs | 3.4 |
| 424625 | AW904466 | Hs.321197 | PDZ domain protein (Drosophila inaD-like | 3.4 |
| 428508 | BE252383 | Hs.184668 | SBBI31 protein | 3.4 |
| 455651 | BE064962 | | gb: RC1-BT0313-130400-016-c02 BT0313 Homo | 3.4 |
| 410555 | U92649 | Hs.64311 | a disintegrin and metalloproteinase doma | 3.4 |
| 447754 | AW073310 | Hs.163533 | Homo sapiens cDNA FLJ14142 fis, clone MA | 3.4 |
| 418636 | AW749855 | | gb: QV4-BT0534-281299-053-c05 BT0534 Homo | 3.4 |
| 404097 | NA | | C5000242*: gi\|9369379\|gb\|AAF87128.1\|AC006 | 3.4 |
| 434205 | AF119861 | Hs.283032 | hypothetical protein PRO2015 | 3.4 |
| 421072 | AI215069 | Hs.89113 | ESTs | 3.4 |
| 402421 | NA | | C1001578*: gi\|6759903\|gb\|AAF28099.1\| (AF1 | 3.4 |
| 405248 | NA | | Target Exon | 3.4 |
| 407638 | AJ404672 | Hs.334483 | hypothetical protein FLJ23571 | 3.4 |
| 403000 | BE247275 | Hs.151787 | U5 snRNP-specific protein, 116 kD | 3.4 |
| 433393 | AF038564 | Hs.98074 | itchy (mouse homolog) E3 ubiquitin prote | 3.4 |
| 432239 | X81334 | Hs.2936 | matrix metalloproteinase 13 (collagenase | 3.4 |
| 458747 | BE618395 | Hs.257391 | hypothetical protein DKFZp761J1523 | 3.4 |
| 442082 | R41823 | Hs.7413 | ESTs; calsyntenin-2 | 3.4 |
| 417974 | AA210765 | | gb: zr90c06.r1 NCI_CGAP_GCB1 Homo sapiens | 3.4 |
| 446002 | AI346468 | Hs.145789 | ESTs | 3.4 |
| 448995 | AI613276 | Hs.5662 | guanine nucleotide binding protein (G pr | 3.4 |
| 436007 | AI247716 | Hs.232168 | ESTs | 3.4 |
| 424698 | AA164366 | Hs.151973 | hypothetical protein FLJ23511 | 3.4 |
| 435202 | AI971313 | Hs.170204 | KIAA0551 protein | 3.4 |
| 410467 | AF102546 | Hs.63931 | dachshund (Drosophila) homolog | 3.3 |
| 405460 | NA | | Target Exon | 3.3 |
| 441826 | AW503603 | Hs.129915 | phosphotriesterase related | 3.3 |
| 453472 | AL037925 | | gb: DKFZp564M037_r1 564 (synonym: hfbr2) | 3.3 |
| 447078 | AW885727 | Hs.301570 | ESTs | 3.3 |
| 441690 | R81733 | Hs.33106 | ESTs | 3.3 |
| 420092 | AA814043 | Hs.88045 | ESTs | 3.3 |
| 418478 | U38945 | Hs.1174 | cyclin-dependent kinase inhibitor2A (me | 3.3 |
| 408908 | BE296227 | Hs.250822 | serine/threonine kinase 15 | 3.3 |
| 414737 | AI160386 | Hs.125087 | ESTs | 3.3 |
| 449650 | AF055575 | Hs.23838 | calcium channel, voltage-dependent, L ty | 3.3 |
| 418912 | NM_000685 | Hs.89472 | angiotensin receptor 1 | 3.3 |
| 436405 | AA160079 | Hs.172932 | Homo sapiens mRNA for partial 3'UTR, seq | 3.3 |
| 453911 | AW503857 | Hs.4007 | Sarcolemmal-associated protein | 3.3 |
| 409361 | NM_005982 | Hs.54416 | sine oculis homeobox (Drosophila) homolo | 3.3 |
| 429548 | AW138872 | Hs.135288 | ESTs | 3.3 |
| 420807 | AA280627 | Hs.57846 | ESTs | 3.3 |
| 409695 | AA296961 | | gb: EST112514 Adrenal gland tumor Homo sa | 3.3 |
| 445189 | AI936450 | Hs.147482 | ESTs | 3.3 |
| 402892 | NA | | Target Exon | 3.3 |
| 426681 | AA994896 | Hs.22514 | ESTs | 3.3 |
| 458722 | AA741545 | Hs.282832 | ESTs, Weakly similar to T24961 hypotheti | 3.3 |
| 409430 | R21945 | Hs.166975 | splicing factor, arginine/serine-rich 5 | 3.3 |
| 443194 | AI954968 | Hs.279009 | matrix Gla protein | 3.3 |
| 445432 | AV653771 | | gb: AV653771 GLC Homo sapiens cDNA clone | 3.3 |
| 410908 | AA121686 | Hs.10592 | ESTs | 3.3 |
| 406151 | NA | | Target Exon | 3.3 |
| 436461 | AW511956 | Hs.293261 | ESTs | 3.3 |
| 411171 | AW820260 | | gb: QV2-ST0296-150200-040-c10 ST0296 Homo | 3.3 |
| 432415 | T16971 | Hs.289014 | ESTs, Weakly similar to A43932 mucin 2 p | 3.3 |
| 439310 | AF086120 | Hs.102793 | ESTs | 3.3 |
| 401575 | NA | | Target Exon | 3.3 |
| 420900 | AL045633 | Hs.44269 | ESTs | 3.3 |
| 445628 | AI344166 | Hs.155743 | ESTs | 3.3 |
| 448243 | AW369771 | Hs.52620 | integrin, beta 8 | 3.3 |
| 445102 | AW204610 | Hs.22270 | ESTs | 3.3 |
| 442118 | AA976718 | Hs.202242 | ESTs | 3.3 |
| 416783 | AA206186 | Hs.79889 | monocyte to macrophage differentiation-a | 3.3 |
| 435039 | AW043921 | Hs.130526 | ESTs | 3.3 |
| 451474 | T70874 | Hs.207636 | ESTs | 3.2 |
| 442559 | T10213 | Hs.159993 | gycosyltransferase | 3.2 |
| 453921 | AI824009 | Hs.44577 | ESTs | 3.2 |
| 420036 | R60336 | Hs.52792 | Homo sapiens mRNA; cDNA DKFZp586I1823 (f | 3.2 |
| 435627 | W88774 | Hs.118370 | ESTs | 3.2 |
| 411598 | BE336654 | Hs.70937 | H3 histone family, member A | 3.2 |
| 446733 | AA863360 | Hs.26040 | ESTs, Weakly similar to fatty acid omega | 3.2 |
| 410153 | BE311926 | Hs.15830 | hypothetical protein FLJ12691 | 3.2 |
| 403637 | NA | | C3001106*: gi\|10047201\|dbj\|BAB13394.1\| (A | 3.2 |
| 405547 | | | NM_018833*: Homo sapiens transporter 2, A | 3.2 |
| 427878 | C05766 | Hs.181022 | CGI-07 protein | 3.2 |
| 451871 | AI821005 | Hs.118599 | ESTs | 3.2 |
| 410313 | R10305 | Hs.185683 | ESTs | 3.2 |
| 416856 | N27833 | Hs.269028 | ESTs, Weakly similar to I38022 hypotheti | 3.2 |
| 449490 | AI652777 | Hs.197069 | ESTs | 3.2 |
| 450506 | NM_004460 | Hs.418 | fibroblast activation protein, alpha | 3.2 |
| 440684 | AI253123 | Hs.127356 | ESTs, Highly similar to S21424 nestin [H | 3.2 |
| 459055 | N23235 | Hs.30567 | ESTs, Weakly similar to B34087 hypotheti | 3.2 |
| 452190 | H26735 | Hs.91668 | Homo sapiens clone PP1498 unknown mRNA | 3.2 |
| 430965 | AA489732 | Hs.154918 | ESTs | 3.2 |
| 405394 | | | Target Exon | 3.2 |
| 424693 | BE169810 | Hs.47557 | ESTs | 3.2 |
| 454265 | H03556 | Hs.300949 | ESTs, Weakly similar to thyroid hormone | 3.2 |
| 437687 | AA765917 | Hs.122840 | ESTs | 3.2 |
| 428372 | AK000684 | Hs.183887 | hypothetical protein FLJ22104 | 3.2 |
| 414083 | AL121282 | Hs.257786 | ESTs | 3.2 |
| 411670 | AW856552 | | gb: RC1-CT0294-080100-012-a04 CT0294 Homo | 3.2 |
| 416283 | NM_005429 | Hs.79141 | vascular endothelial growth factor C | 3.2 |
| 437488 | AA758239 | Hs.180330 | ESTs | 3.2 |
| 428398 | AI249368 | Hs.98558 | ESTs | 3.2 |
| 452042 | H38857 | Hs.243901 | Homo sapiens cDNA FLJ20738 fis, clone HE | 3.2 |
| 421477 | AI904743 | Hs.104650 | hypothetical protein FLJ10292 | 3.2 |
| 438078 | AI016377 | Hs.131693 | ESTs | 3.2 |
| 448816 | AB033052 | Hs.22151 | KIAA1226 protein | 3.2 |
| 419519 | AI198719 | Hs.176376 | ESTs | 3.2 |
| 404580 | | | NM_014112*: Homo sapiens trichorhinophala | 3.2 |
| 447046 | AA326187 | Hs.17170 | G protein-coupled receptor 4 | 3.2 |
| 457473 | AW974903 | Hs.291231 | ESTs | 3.1 |
| 429838 | AW904907 | Hs.30732 | hypothetical protein FLJ13409; KIAA1711 | 3.1 |
| 459702 | AI204995 | | gb: an03c03.x1 Stratagene schizo brain S1 | 3.1 |
| 400195 | NA | | NM_007057*: Homo sapiens ZW10 interactor | 3.1 |
| 417860 | AW408557 | Hs.235498 | hypothetical protein FLJ14075 | 3.1 |
| 417995 | AW974175 | Hs.188751 | ESTs, Weakly similar to MAPB_HUMAN MICRO | 3.1 |
| 422589 | AA312735 | Hs.30512 | Homo sapiens mRNA for KIAA0556 protein, | 3.1 |
| 435870 | AA701327 | Hs.17949 | ESTs | 3.1 |
| 440801 | AA906366 | Hs.190535 | ESTs | 3.1 |
| 426274 | D38122 | Hs.2007 | tumor necrosis factor (ligand) superfami | 3.1 |
| 423728 | AW891294 | Hs.132136 | solute carrier family 4, sodium bicarbon | 3.1 |
| 439677 | R82331 | Hs.164599 | ESTs | 3.1 |
| 452834 | AI638627 | Hs.105685 | KIAA1688 protein | 3.1 |
| 431349 | AA503653 | Hs.156942 | ESTs, Moderately similar to ALU2_HUMAN A | 3.1 |
| 417576 | AA339449 | Hs.82285 | phosphoribosylglycinamide formyltransfer | 3.1 |
| 430264 | AA470519 | | gb: nc71f10.s1 NCI_CGAP_Pr1 Homo sapiens | 3.1 |
| 418827 | BE327311 | Hs.47166 | HT021 | 3.1 |
| 410835 | AW806906 | | gb: QV4-ST0023-160400-172-d12 ST0023 Homo | 3.1 |
| 426269 | H15302 | Hs.168950 | Homo sapiens mRNA; cDNA DKFZp566A1046 (f | 3.1 |
| 405336 | NA | | Target Exon | 3.1 |
| 437783 | AI683150 | Hs.201550 | ESTs, Weakly similar to ALU1_HUMAN ALU S | 3.1 |
| 440931 | AI583052 | Hs.270058 | ESTs | 3.1 |
| 455945 | BE160636 | | gb: PM1-HT0422-291299-002-c08 HT0422 Homo | 3.1 |
| 430437 | AI768801 | Hs.169943 | Homo sapiens cDNA FLJ13569 fis, clone PL | 3.1 |
| 405848 | NA | | Target Exon | 3.1 |
| 455685 | BE066976 | | gb: PM0-BT0340-211299-003-c12 BT0340 Homo | 3.1 |
| 406970 | M29994 | | gb: Human alpha-I spectrin gene, exon 12. | 3.1 |
| 409602 | W26713 | Hs.256972 | ESTs | 3.1 |
| 423518 | D45027 | Hs.129732 | R3H domain (binds single-stranded nuclei | 3.1 |
| 425653 | AI065104 | Hs.249718 | ESTs, Weakly similar to A46010 X-linked | 3.1 |
| 426326 | BE165753 | Hs.250528 | Homo sapiens , clone IMAGE: 4098694, mRNA, | 3.1 |
| 433805 | AA706910 | Hs.112742 | ESTs | 3.1 |
| 437152 | AL050027 | | gb: Homo sapiens mRNA; cDNA DKFZp566C0324 | 3.1 |
| 448602 | AI541305 | Hs.48778 | niban protein | 3.1 |
| 452844 | AW407181 | Hs.218377 | Homo sapiens cDNA FLJ11927 fis, clone HE | 3.1 |
| 407366 | AF026942 | | gb: Homo sapiens cig33 mRNA, partial sequ | 3.1 |
| 408254 | AW807227 | | gb: MR4-ST0062-180200-001-e10 ST0062 Homo | 3.1 |
| 424085 | NM_002914 | Hs.139226 | replication factor C (activator 1) 2 (40 | 3.1 |
| 416790 | R83066 | Hs.7043 | succinate-CoA ligase, GDP-forming, alpha | 3.1 |
| 420020 | BE295866 | Hs.94382 | adenosine kinase | 3.1 |
| 426119 | W94997 | Hs.189917 | ESTs | 3.1 |
| 426968 | U07616 | Hs.173034 | amphiphysin (Stiff-Mann syndrome with br | 3.1 |
| 457421 | AL117431 | Hs.112165 | Homo sapiens cDNA FLJ12198 fis, clone MA | 3.1 |
| 453403 | BE466639 | Hs.61779 | Homo sapiens cDNA FLJ13591 fis, clone PL | 3.1 |
| 454141 | AW138413 | Hs.139336 | ATP-binding cassette, sub-family C (CFTR | 3.1 |
| 426650 | AA382814 | | gb: EST96097 Testis I Homo sapiens cDNA 5 | 3.1 |
| 450865 | AI248013 | Hs.106532 | ESTs, Weakly similar to I38588 reverse t | 3.1 |
| 407993 | AW135274 | Hs.12433 | ESTs | 3.1 |
| 446466 | H38026 | Hs.308 | arrestin 3, retinal (X-arrestin) | 3.1 |
| 457888 | BE219794 | Hs.293471 | ESTs | 3.1 |
| 420058 | AK001423 | Hs.94694 | Homo sapiens cDNA FLJ10561 fis, clone NT | 3.0 |
| 409248 | AB033035 | Hs.51965 | KIAA1209 protein | 3.0 |
| 452747 | BE153855 | Hs.61460 | Ig superfamily receptor LNIR | 3.0 |
| 418926 | AA232658 | Hs.105794 | UDP-glucose: glycoprotein glucosyltransfe | 3.0 |
| 419346 | AI830417 | Hs.44143 | polybromo 1 | 3.0 |
| 429826 | N93266 | Hs.40747 | ESTs | 3.0 |
| 435147 | AL133731 | Hs.4774 | Homo sapiens mRNA; cDNA DKFZp761C1712 (f | 3.0 |
| 420139 | NM_005357 | Hs.95351 | lipase, hormone-sensitive | 3.0 |
| 405609 | NA | | ENSP00000241065*: CDNA | 3.0 |
| 404274 | | | NM_002944*: Homo sapiens v-ros avian UR2 | 3.0 |
| 449777 | AI971362 | Hs.231945 | ESTs | 3.0 |
| 415459 | H07118 | Hs.6099 | ESTs | 3.0 |
| 415245 | N59650 | Hs.27252 | ESTs | 3.0 |
| 406291 | NA | | Target Exon | 3.0 |
| 414210 | BE383592 | | gb: 601297871F1 NIH_MGC_19 Homo sapiens c | 3.0 |
| 432055 | AW972359 | Hs.293334 | ESTs | 3.0 |
| 442246 | AI791988 | Hs.129115 | ESTs | 3.0 |
| 451353 | N21043 | Hs.42932 | ESTs | 3.0 |
| 451177 | AI969716 | Hs.13034 | ESTs | 3.0 |
| 418026 | BE379727 | Hs.83213 | fatty acid binding protein 4, adipocyte | 3.0 |
| 401326 | NA | | C10000447*: gi\|1168375\|sp\|P43467\|AGA1_PED | 3.0 |
| 409920 | BE169746 | Hs.12504 | likely ortholog of mouse Arkadia | 3.0 |
| 432887 | AI926047 | Hs.162859 | ESTs | 3.0 |
| 411789 | AF245505 | Hs.72157 | DKFZP564I1922 protein | 3.0 |
| 401045 | | | C11001883*: gi\|6753278\|ref\|NP_033938.1\| c | 3.0 |
| 432162 | AA584062 | Hs.272798 | hypothetical protein FLJ20413 | 3.0 |
| 434627 | AI221894 | Hs.39311 | ESTs | 3.0 |
| 442611 | BE077155 | Hs.177537 | hypothetical protein DKFZp761B1514 | 3.0 |
| 425477 | AW958879 | Hs.270535 | ESTs | 3.0 |
| 457183 | H91882 | Hs.118569 | DvI-binding protein IDAX (inhibition of | 3.0 |
| 433014 | NM_014711 | Hs.279912 | KIAA0419 gene product | 3.0 |
| 415542 | R13474 | Hs.290263 | ESTs, Weakly similar to I38022 hypotheti | 3.0 |
| 416173 | R52782 | | gb: yg99d09.r1 Soares infant brain 1NIB H | 3.0 |
| 408155 | AB014528 | Hs.43133 | KIAA0628 gene product | 3.0 |
| 453913 | AW004683 | Hs.78934 | mutS (E. coli) homolog 2 (colon cancer, | 3.0 |
| 435495 | AI754212 | Hs.21951 | Homo sapiens Xq pseudoautosomal region; | 3.0 |
| 423629 | AW021173 | Hs.18612 | Homo sapiens cDNA: FLJ21909 fis, clone H | 3.0 |
| 411836 | AW901879 | Hs.314453 | ESTs | 3.0 |
| 415030 | D31118 | Hs.191735 | hypothetical protein MGC10520 | 3.0 |
| 419606 | AW294795 | Hs.198529 | ESTs | 3.0 |
| 440310 | AA878939 | Hs.125406 | ESTs | 3.0 |
| 443608 | AI375957 | Hs.289074 | F-box only protein 22 | 3.0 |
| 414140 | AA281279 | Hs.23317 | hypothetical protein FLJ14681 | 3.0 |
| 444781 | NM_014400 | Hs.11950 | GPI-anchored metastasis-associated prote | 3.0 |
| 435393 | AA701259 | Hs.189299 | ESTs | 3.0 |
| 454071 | AI041793 | Hs.42502 | ESTs | 3.0 |
| 446922 | BE175605 | | gb: RC5-HT0580-100500-022-H07 HT0580 Homo | 3.0 |
| 448062 | AW295923 | Hs.255472 | KIAA1843 protein | 3.0 |
| 427711 | M31659 | Hs.180408 | solute carrier family 25 (mitochondrial | 3.0 |
| 450382 | AA397658 | Hs.60257 | Homo sapiens cDNA FLJ13598 fis, clone PL | 3.0 |
| 424866 | W01938 | Hs.337243 | ESTs, Weakly similar to ALU7_HUMAN ALU S | 2.9 |
| 433043 | W57554 | Hs.125019 | lymphoid nuclear protein (LAF-4) mRNA | 2.9 |
| 420802 | U22376 | Hs.1334 | v-myb avian myeloblastosis viral oncogen | 2.9 |
| 445625 | BE246743 | Hs.288529 | hypothetical protein FLJ22635 | 2.9 |
| 403677 | NA | | C4001462: gi\|4887715\|gb\|AAA79329.2\| (L088 | 2.9 |
| 411093 | BE067650 | | gb: MR4-BT0358-090300-003-e01 BT0358 Homo | 2.9 |
| 435255 | W87434 | Hs.106015 | ESTs, Moderately similar to ALU1_HUMAN A | 2.9 |
| 443127 | BE568102 | Hs.180312 | mitochondrial ribosomal protein S16 | 2.9 |
| 448104 | AI674818 | Hs.316433 | Homo sapiens cDNA FLJ11375 fis, clone HE | 2.9 |
| 427315 | AA179949 | Hs.175563 | Homo sapiens mRNA; cDNA DKFZp564N0763 (f | 2.9 |
| 430414 | AW365665 | Hs.120388 | ESTs | 2.9 |
| 423600 | AI633559 | Hs.310359 | ESTs | 2.9 |
| 458562 | N34128 | Hs.145268 | ESTs | 2.9 |
| 402109 | NA | | Target Exon | 2.9 |
| 429629 | BE501732 | Hs.30622 | Homo sapiens cDNA FLJ13010 fis, clone NT | 2.9 |
| 442295 | AI827248 | Hs.224398 | Homo sapiens cDNA FLJ11469 fis, clone HE | 2.9 |
| 419752 | AA249573 | Hs.152618 | ESTs, Moderately similar to ZN91_HUMAN Z | 2.9 |
| 404721 | | | NM_005596*: Homo sapiens nuclear factor I | 2.9 |
| 445107 | AI208121 | Hs.147313 | ESTs, Weakly similar to I38022 hypotheti | 2.9 |
| 401987 | | | NM_002737*: Homo sapiens protein kinase C | 2.9 |
| 430566 | AA481282 | Hs.190149 | ESTs | 2.9 |
| 444517 | AI939339 | Hs.146883 | ESTs | 2.9 |
| 445563 | AW873606 | Hs.149006 | ESTs | 2.9 |
| 427691 | AW194426 | Hs.20726 | ESTs | 2.9 |
| 456561 | AI868634 | Hs.246358 | ESTs, Weakly similar to T32250 hypotheti | 2.9 |
| 401458 | | | Target Exon | 2.9 |
| 421039 | NM_003478 | Hs.101299 | cullin 5 | 2.9 |
| 459504 | BE514127 | | gb: 601315974 F1 NIH_MGC_8 Homo sapiens cD | 2.9 |
| 424962 | NM_012288 | Hs.153954 | TRAM-like protein | 2.9 |
| 409617 | BE003760 | Hs.55209 | Homo sapiens mRNA; cDNA DKFZp434K0514 (f | 2.9 |
| 416931 | D45371 | Hs.80485 | adipose most abundant gene transcript 1 | 2.9 |
| 413221 | BE161151 | | gb: PM0-HT0425-141299-001-F08 HT0425 Homo | 2.9 |
| 409732 | NM_016122 | Hs.56148 | NY-REN-58 antigen | 2.9 |
| 433687 | AA743991 | | gb: ny57g01.s1 NCI_CGAP_Pr18 Homo sapiens | 2.9 |
| 434340 | AI193043 | Hs.128685 | ESTs, Weakly similar to T17226 hypotheti | 2.9 |
| 454529 | Z45439 | Hs.270425 | ESTs | 2.9 |
| 421379 | Y15221 | Hs.103982 | small inducible cytokine subfamily B (Cy | 2.9 |
| 452291 | AF015592 | Hs.28853 | CDC7 (cell division cycle 7, S. cerevisi | 2.9 |
| 457402 | AW452648 | Hs.149342 | activation-induced cytidine deaminase | 2.9 |
| 449051 | AW961400 | Hs.333526 | HER2 receptor tyrosine kinase (c-erb-b2, | 2.9 |
| 408761 | AA057264 | Hs.238936 | ESTs, Weakly similar to (defline not ava | 2.9 |
| 401093 | | | C12000586*: gi\|6330167\|dbj\|BAA86477.1\| (A | 2.9 |
| 435061 | AI651474 | Hs.163944 | ESTs | 2.9 |
| 447985 | AI681475 | Hs.200949 | ESTs | 2.9 |
| 449340 | AW235786 | Hs.195359 | hypothetical protein MGC10954 | 2.9 |
| 426384 | AI472078 | Hs.303662 | ESTs | 2.9 |
| 411905 | BE265067 | | gb: 601193893F1 NIH_MGC_7 Homo sapiens cD | 2.9 |
| 405953 | NA | | Target Exon | 2.8 |
| 420854 | AW296927 | | gb: UI-H-BW0-ajc-c-07-0-UI.s1 NCI_CGAP_Su | 2.8 |
| 434265 | AA846811 | Hs.130554 | Homo sapiens cDNA: FLJ23089 fis, clone L | 2.8 |
| 428365 | AA295331 | Hs.183861 | Homo sapiens cDNA FLJ20042 fis, clone CO | 2.8 |
| 442861 | AA243837 | Hs.57787 | ESTs | 2.8 |
| 448337 | AW206453 | Hs.3782 | ESTs | 2.8 |
| 452554 | AW452434 | Hs.58006 | ESTs, Weakly similar to ALU5_HUMAN ALU S | 2.8 |
| 412248 | BE176480 | | gb: RC3-HT0585-160300-022-c02 HT0585 Homo | 2.8 |
| 449450 | AL039852 | Hs.49136 | ESTs, Moderately similar to ALU7_HUMAN A | 2.8 |
| 434757 | AI038997 | Hs.132921 | ESTs | 2.8 |
| 409038 | T97490 | Hs.50002 | small inducible cytokine subfamily A (Cy | 2.8 |
| 454545 | AW806899 | | gb: QV4-ST0023-160400-172-c12 ST0023 Homo | 2.8 |
| 439842 | AI910896 | Hs.132413 | ESTs | 2.8 |
| 428479 | Y00272 | Hs.184572 | cell division cycle 2, G1 to S and G2 to | 2.8 |
| 424800 | AL035588 | Hs.153203 | MyoD family inhibitor | 2.8 |
| 411086 | BE070800 | | gb: RC3-BT0502-251199-011-c07 BT0502 Homo | 2.8 |
| 400250 | NA | | Eos Control | 2.8 |
| 449168 | NM_016206 | Hs.23142 | colon carcinoma related protein | 2.8 |
| 456482 | AA485224 | | gb: aa41b12.s1 NCI_CGAP_GCB1 Homo sapiens | 2.8 |
| 426044 | AA502490 | Hs.336695 | ESTs | 2.8 |
| 431854 | AA383550 | Hs.271699 | polymerase (DNA directed) iota | 2.8 |
| 405873 | NA | | Target Exon | 2.8 |
| 440400 | AA994364 | Hs.125594 | ESTs, Weakly similar to T25472 hypotheti | 2.8 |
| 458265 | AI075375 | Hs.128193 | ESTs, Weakly similar to IRX2_HUMAN IROQU | 2.8 |
| 413708 | BE158791 | | gb: IL2-HT0397-091299-025-D02 HT0397 Homo | 2.8 |
| 423739 | AA398155 | Hs.97600 | ESTs | 2.8 |
| 424408 | AI754813 | Hs.146428 | collagen, type V, alpha 1 | 2.8 |
| 453096 | AW294631 | Hs.11325 | ESTs | 2.8 |
| 421825 | AA298758 | Hs.183747 | ESTs, Moderately similar to CALB_HUMAN C | 2.8 |
| 417742 | R64719 | | gb: EST22d11 WATM1 Homo sapiens cDNA clon | 2.8 |
| 402765 | | | C1003621*: gi\|12407405\|gb\|AAG53491.1\|AF22 | 2.8 |
| 444378 | R41339 | Hs.12569 | ESTs | 2.8 |
| 419172 | AW338625 | Hs.22120 | ESTs | 2.8 |
| 401497 | | | Target Exon | 2.8 |
| 402376 | | | C19000763*: gi\|1363912\|pir\|\|JC4296 ring f | 2.8 |
| 405041 | NA | | C3001706*: gi\|1345652\|sp\|P15989\|CA36_CHIC | 2.8 |
| 408758 | NM_003686 | Hs.47504 | exonuclease 1 | 2.8 |
| 431917 | D16181 | Hs.2868 | peripheral myelin protein 2 | 2.8 |
| 437583 | AA761190 | Hs.244627 | ESTs | 2.8 |
| 453737 | AA744862 | Hs.194293 | ESTs, Weakly similar to I54374 gene NF2 | 2.8 |
| 458094 | AF086325 | | gb: Homo sapiens full length insert cDNA | 2.8 |
| 401283 | NA | | Target Exon | 2.8 |
| 410784 | AW803201 | | gb: IL2-UM0077-070500-080-E06 UM0077 Homo | 2.8 |
| 417601 | NM_014735 | Hs.82292 | KIAA0215 gene product | 2.8 |
| 418236 | AW994005 | Hs.337534 | ESTs | 2.8 |
| 435532 | AW291488 | Hs.117305 | Homo sapiens , clone IMAGE: 3682908, mRNA | 2.8 |
| 454714 | AW815098 | | gb: QV4-ST0212-091199-023-f10 ST0212 Homo | 2.8 |
| 418629 | BE247550 | Hs.86859 | growth factor receptor-bound protein 7 | 2.8 |
| 442101 | AI651930 | Hs.135684 | ESTs | 2.8 |
| 405080 | AK000375 | Hs.88820 | HDCMC28P protein | 2.8 |
| 414661 | T97401 | Hs.21929 | ESTs | 2.8 |
| 425589 | AI650633 | Hs.143688 | Homo sapiens cDNA: FLJ23031 fis, clone L | 2.8 |
| 429638 | AI916662 | Hs.211577 | kinectin 1 (kinesin receptor) | 2.7 |
| 428824 | W23624 | Hs.173059 | ESTs | 2.7 |
| 421566 | NM_000399 | Hs.1395 | early growth response 2 (Krox-20 (Drosop | 2.7 |
| 414596 | BE386870 | | gb: 601275271F1 NIH_MGC_20 Homo sapiens c | 2.7 |
| 440868 | R79707 | Hs.263339 | ESTs, Moderately similar to I38022 hypot | 2.7 |
| 452943 | BE247449 | Hs.31082 | hypothetical protein FLJ10525 | 2.7 |
| 443772 | AV646449 | Hs.282872 | ESTs | 2.7 |
| 432361 | AI378562 | Hs.159585 | ESTs | 2.7 |
| 430375 | AW371048 | Hs.93758 | H4 histone family, member H | 2.7 |
| 406504 | NA | | C5000558: gi\|4504675\|ref\|NP_002175.1\|int | 2.7 |
| 423279 | AW959861 | Hs.290943 | ESTs | 2.7 |
| 424871 | NM_004525 | Hs.153595 | low density lipoprotein-related protein | 2.7 |
| 453619 | H87648 | Hs.33922 | Homo sapiens , clone MGC: 9084, mRNA, comp | 2.7 |
| 423961 | D13666 | Hs.136348 | osteoblast specific factor 2 (fasciclin | 2.7 |
| 422156 | N34524 | | gb: yy56d10.s1 Soares_multiple_sclerosis_{—} | 2.7 |
| 415752 | BE314524 | Hs.78776 | putative transmembrane protein | 2.7 |
| 419987 | NM_005014 | Hs.94070 | osteomodulin | 2.7 |
| 406182 | NA | | Target Exon | 2.7 |
| 416495 | X69970 | Hs.79350 | RYK receptor-like tyrosine kinase | 2.7 |
| 444701 | AI916512 | Hs.198394 | ESTs | 2.7 |
| 408171 | AA301228 | Hs.43299 | hypothetical protein FLJ12890 | 2.7 |
| 430153 | AW968128 | Hs.336679 | ESTs | 2.7 |
| 413383 | AA128978 | Hs.154706 | hypothetical protein FLJ14917 | 2.7 |
| 414831 | M31158 | Hs.77439 | protein kinase, cAMP-dependent, regulato | 2.7 |
| 413278 | BE563085 | Hs.833 | interferon-stimulated protein, 15 kDa | 2.7 |
| 433132 | AB026264 | Hs.284245 | hypothetical protein IMPACT | 2.7 |
| 437030 | AA742577 | Hs.303781 | EST | 2.7 |
| 439031 | AF075079 | | gb: Homo sapiens full length insert cDNA | 2.7 |
| 449532 | W74653 | Hs.271593 | ESTs, Moderately similar to A47582 B-cel | 2.7 |
| 406153 | | | Target Exon | 2.7 |
| 406625 | Y13647 | Hs.119597 | stearoyl-CoA desaturase (delta-9-desatur | 2.7 |
| 444698 | AI188139 | Hs.147050 | ESTs | 2.7 |
| 432328 | AI572739 | Hs.195471 | 6-phosphofructo-2-kinase/fructose-2,6-bi | 2.7 |
| 429628 | H09604 | Hs.13268 | ESTs | 2.7 |
| 420149 | AA255920 | Hs.88095 | ESTs | 2.7 |
| 431207 | AA495925 | Hs.9394 | ESTs | 2.7 |
| 438394 | BE379623 | Hs.27693 | peptidylprolyl isomerase (cyclophilin)-I | 2.7 |
| 443304 | AI050073 | Hs.135338 | ESTs | 2.7 |
| 427660 | AI741320 | Hs.114121 | Homo sapiens cDNA: FLJ23228 fis, clone C | 2.7 |
| 408460 | AA054726 | Hs.285574 | ESTs | 2.7 |
| 416515 | N91716 | Hs.194140 | ESTs, Weakly similar to I38022 hypotheti | 2.7 |
| 429922 | Z97630 | Hs.226117 | H1 histone family, member 0 | 2.7 |
| 418203 | X54942 | Hs.83758 | CDC28 protein kinase 2 | 2.7 |
| 439509 | AF086332 | Hs.58314 | ESTs | 2.7 |
| 402184 | NA | | ENSP00000245238*: CDNA FLJ10922 fis, clon | 2.7 |
| 450496 | AW449251 | Hs.257131 | ESTs | 2.7 |
| 451963 | AI825440 | Hs.224952 | ESTs | 2.7 |
| 457938 | AI373638 | Hs.133900 | ESTs | 2.7 |
| 441541 | AA938663 | Hs.199828 | ESTs | 2.7 |
| 441111 | AI806867 | Hs.126594 | ESTs | 2.7 |
| 423020 | AA383092 | Hs.1608 | replication protein A3 (14 kD) | 2.7 |
| 445354 | AV653485 | Hs.6390 | Homo sapiens clone FLB3344 PRO0845 mRNA, | 2.7 |
| 427961 | AW293165 | Hs.143134 | ESTs | 2.7 |
| 410889 | X91662 | Hs.66744 | twist (Drosophila) homolog (acrocephalos | 2.7 |
| 445234 | AW137636 | Hs.146059 | ESTs | 2.7 |
| 413903 | AA496493 | Hs.23136 | ESTs | 2.7 |
| 406069 | NA | | Target Exon | 2.7 |
| 447410 | AI470235 | Hs.172698 | EST | 2.7 |
| 401256 | NA | | NM_024089*: Homo sapiens hypothetical pro | 2.7 |
| 415139 | AW975942 | Hs.48524 | ESTs | 2.7 |
| 420218 | AW958037 | Hs.286 | ribosomal protein L4 | 2.7 |
| 455511 | BE144762 | | gb: CM0-HT0180-041099-065-b04 HT0180 Homo | 2.7 |
| 438825 | BE327427 | Hs.79953 | ESTs | 2.6 |
| 409564 | AA045857 | Hs.54943 | fracture callus 1 (rat) homolog | 2.6 |
| 452837 | AL121053 | Hs.5534 | Homo sapiens cDNA FLJ12961 fis, clone NT | 2.6 |
| 434876 | AF160477 | Hs.61460 | Ig superfamily receptor LNIR | 2.6 |
| 421565 | AK001122 | Hs.105859 | hypothetical protein FLJ10260 | 2.6 |
| 453279 | AW893940 | Hs.59698 | ESTs | 2.6 |
| 430785 | Z30201 | | gb: HHEA22G Atrium cDNA library Human hea | 2.6 |
| 456986 | D38299 | Hs.170917 | prostaglandin E receptor 3 (subtype EP3) | 2.6 |
| 433068 | NM_006456 | Hs.288215 | sialyltransferase | 2.6 |
| 421952 | AA300900 | Hs.98849 | ESTs, Moderately similar to AF161511 1 H | 2.6 |
| 429208 | AA447990 | Hs.190478 | ESTs | 2.6 |
| 430733 | AW975920 | Hs.283361 | ESTs | 2.6 |
| 441720 | AI346487 | Hs.28739 | ESTs | 2.6 |
| 418986 | AI123555 | Hs.81796 | ESTs | 2.6 |
| 432481 | AW451645 | Hs.151504 | Homo sapiens cDNA FLJ11973 fis, clone HE | 2.6 |
| 434338 | AW754311 | | gb: CM1-CT0337-141299-068-f07 CT0337 Homo | 2.6 |
| 417061 | AI675944 | Hs.188691 | Homo sapiens cDNA FLJ12033 fis, clone HE | 2.6 |
| 410530 | M25809 | Hs.64173 | ATPase, H transporting, lysosomal (vacuo | 2.6 |
| 456672 | AK002016 | Hs.114727 | Homo sapiens , clone MGC: 16327, mRNA, com | 2.6 |
| 425071 | NM_013989 | Hs.154424 | deiodinase, iodothyronine, type II | 2.6 |
| 408868 | AW292286 | Hs.255058 | ESTs | 2.6 |
| 451531 | AA018311 | Hs.114762 | ESTs | 2.6 |
| 405822 | | | Target Exon | 2.6 |
| 418301 | AW976201 | Hs.53913 | hypothetical protein FLJ10252 | 2.6 |
| 417315 | AI080042 | Hs.336901 | ribosomal protein S24 | 2.6 |
| 434699 | AA643687 | Hs.149425 | Homo sapiens cDNA FLJ11980 fis, clone HE | 2.6 |
| 443204 | AW205878 | Hs.29643 | Homo sapiens cDNA FLJ13103 fis, clone NT | 2.6 |
| 405638 | | | Target Exon | 2.6 |
| 452542 | AW812256 | | gb: RC0-ST0174-191099-031-a07 ST0174 Homo | 2.6 |
| 403943 | | | C5000355: gi\|4503225\|ref\|NP_000765.1\| cyt | 2.6 |
| 404535 | Z25884 | Hs.121483 | chloride channel 1, skeletal muscle (Th | 2.6 |
| 402800 | NA | | Target Exon | 2.6 |
| 449144 | AI989503 | Hs.233405 | ESTs | 2.6 |
| 454934 | AW846080 | Hs.314324 | ESTs | 2.6 |
| 424717 | H03754 | Hs.152213 | wingless-type MMTV integration site fami | 2.6 |
| 428303 | AW974476 | Hs.183601 | regulator of G-protein signalling 16 | 2.6 |
| 427970 | AA418187 | Hs.330515 | ESTs | 2.6 |
| 450638 | AK001826 | Hs.25245 | hypothetical protein FLJ11269 | 2.6 |
| 453034 | BE246010 | Hs.271468 | Homo sapiens mRNA for FLJ00038 protein, | 2.6 |
| 455097 | AW855802 | | gb: RC1-CT0279-170200-023-d08 CT0279 Homo | 2.6 |
| 427317 | AB028955 | Hs.175780 | KIAA1032 protein | 2.6 |
| 408875 | NM_015434 | Hs.48604 | DKFZP434B168 protein | 2.6 |
| 427510 | Z47542 | Hs.179312 | small nuclear RNA activating complex, po | 2.6 |
| 423201 | NM_000163 | Hs.125180 | growth hormone receptor | 2.6 |
| 406271 | | | Target Exon | 2.6 |
| 442696 | BE566962 | Hs.7063 | Homo sapiens cDNA: FLJ20913 fis, clone A | 2.6 |
| 454018 | AW016892 | Hs.100855 | ESTs | 2.6 |
| 435420 | AI928513 | Hs.59203 | ESTs | 2.6 |
| 434398 | AA121098 | Hs.3838 | serum-inducible kinase | 2.6 |
| 455708 | BE069326 | | gb: QV3-BT0381-170100-060-g03 BT0381 Homo | 2.6 |
| 439347 | W24320 | Hs.102941 | Homo sapiens cDNA: FLJ21531 fis, clone C | 2.6 |
| 407523 | X64984 | | gb: H. sapiens mRNA HTPCRX10 for olfactory | 2.6 |
| 425101 | AA830431 | Hs.180811 | ESTs | 2.6 |
| 435153 | AA668763 | Hs.291939 | ESTs | 2.6 |
| 409139 | AI681917 | Hs.3321 | ESTs, Highly similar to IRX1_HUMAN IROQU | 2.6 |
| 455100 | BE160198 | | gb: QV1-HT0413-010200-059-h03 HT0413 Homo | 2.6 |
| 414612 | BE274552 | Hs.76578 | protein inhibitor of activated STAT3 | 2.6 |
| 440283 | AI732892 | Hs.190489 | ESTs | 2.6 |
| 423025 | AA831267 | Hs.12244 | hypothetical protein FLJ20097 | 2.6 |
| 431473 | AA825686 | Hs.321176 | ESTs, Weakly similar to S65824 reverse t | 2.6 |
| 404440 | | | NM_021048: Homo sapiens melanoma antigen, | 2.6 |
| 403388 | NA | | C3001398*: gi\|12248917\|dbj\|BAB20375.1\| (A | 2.6 |
| 403775 | NA | | Target Exon | 2.6 |
| 405037 | NA | | NM_021628*: Homo sapiens arachidonate lip | 2.6 |
| 407447 | AF290544 | | gb: Homo sapiens aminopeptidase mRNA, par | 2.6 |
| 420952 | AA282067 | Hs.88972 | ESTs, Moderately similar to A46010 X-lin | 2.6 |
| 435447 | AI872932 | | gb: wm72e03.x1 NCI_CGAP_Ut2 Homo sapiens | 2.6 |
| 440202 | AW516211 | Hs.125300 | ring finger protein 21, interferon-respo | 2.6 |
| 445854 | AI702885 | Hs.145568 | ESTs | 2.6 |
| 421247 | BE391727 | Hs.102910 | general transcription factor IIH, polype | 2.6 |
| 414870 | N72264 | Hs.300670 | KIAA1204 protein | 2.6 |
| 457411 | AW085961 | Hs.130093 | ESTs | 2.6 |
| 424676 | Y08565 | Hs.151678 | UDP-N-acetyl-alpha-D-galactosamine: polyp | 2.6 |
| 404443 | | | C8001428*: gi\|6572242\|emb\|CAB62951.1\| (Z9 | 2.6 |
| 452268 | NM_003512 | Hs.28777 | H2A histone family, member L | 2.6 |
| 430832 | AI073913 | Hs.100686 | ESTs, Weakly similar to JE0350 Anterior | 2.6 |
| 444779 | AI192105 | Hs.147170 | ESTs | 2.6 |
| 408633 | AW963372 | Hs.46677 | PRO2000 protein | 2.6 |
| 459089 | F13036 | Hs.27373 | Homo sapiens mRNA; cDNA DKFZp564O1763 (f | 2.6 |
| 447197 | R36075 | | gb: yh88b01.s1 Soares placenta Nb2HP Homo | 2.6 |
| 454111 | AW081681 | Hs.269064 | ESTs, Weakly similar to T42689 hypotheti | 2.6 |
| 411165 | NM_000169 | Hs.69089 | galactosidase, alpha | 2.6 |
| 406922 | S70284 | | gb: stearoyl-CoA desaturase [human, adipo | 2.6 |
| 456045 | H62943 | Hs.154188 | ESTs | 2.6 |
| 413111 | BE065837 | | gb: RC2-BT0318-110100-012-g12 BT0318 Homo | 2.6 |
| 423123 | NM_012247 | Hs.124027 | SELENOPHOSPHATE SYNTHETASE; Human selen | 2.6 |
| 432201 | AI538613 | Hs.298241 | Transmembrane protease, serine 3 | 2.5 |
| 427032 | AF012023 | Hs.173274 | integrin cytoplasmic domain-associated p | 2.5 |
| 445417 | AK001058 | Hs.12680 | Homo sapiens cDNA FLJ10196 fis, clone HE | 2.5 |
| 422225 | BE245652 | Hs.118281 | zinc finger protein 266 | 2.5 |
| 428330 | L22524 | Hs.2256 | matrix metalloproteinase 7 (MMP7; uterin | 2.5 |
| 410011 | AB020641 | Hs.57856 | PFTAIRE protein kinase 1 | 2.5 |
| 426310 | NM_000909 | Hs.169266 | neuropeptide Y receptor Y1 | 2.5 |
| 419589 | AW973708 | Hs.201925 | Homo sapiens cDNA FLJ13446 fis, clone PL | 2.5 |
| 437770 | AA767881 | Hs.122897 | ESTs | 2.5 |
| 442818 | AK001741 | Hs.8739 | hypothetical protein FLJ10879 | 2.5 |
| 414251 | AL042306 | Hs.97689 | VASA protein | 2.5 |
| 428301 | AW628666 | Hs.98440 | ESTs, Weakly similar to I38022 hypotheti | 2.5 |
| 431956 | AK002032 | Hs.272245 | Homo sapiens cDNA FLJ11170 fis, clone PL | 2.5 |
| 455732 | BE080908 | | gb: QV1-BT0631-280200-084-h07 BT0631 Homo | 2.5 |
| 458624 | AI362790 | Hs.278639 | KIAA1684 protein; likely homolog of mous | 2.5 |
| 428257 | BE394723 | Hs.275243 | S100 calcium-binding protein A6 (calcycl | 2.5 |
| 418054 | NM_002318 | Hs.83354 | lysyl oxidase-like 2 | 2.5 |
| 458652 | AW375610 | Hs.117102 | hypothetical protein FLJ13046 similar to | 2.5 |
| 458012 | AI424899 | Hs.188211 | ESTs | 2.5 |
| 422996 | BE091089 | | gb: PM4-BT0724-130400-006-c07 BT0724 Homo | 2.5 |
| 410804 | U64820 | Hs.66521 | Machado-Joseph disease (spinocerebellar | 2.5 |
| 457211 | AW972565 | Hs.32399 | ESTs, Weakly similar to S51797 vasodilat | 2.5 |
| 440029 | AW089705 | Hs.293711 | ESTs, Weakly similar to S64329 probable | 2.5 |
| 448141 | AI471598 | Hs.197531 | ESTs | 2.5 |
| 409163 | AA065081 | | gb: zm13a03.s1 Stratagene pancreas (93720 | 2.5 |
| 431385 | BE178536 | Hs.11090 | membrane-spanning 4-domains, subfamily A | 2.5 |
| 414569 | AF109298 | Hs.118258 | prostate cancer associated protein 1 | 2.5 |
| 439963 | AW247529 | Hs.6793 | platelet-activating factor acetylhydrola | 2.5 |
| 455935 | BE158687 | | gb: CM0-HT0395-280100-169-b09 HT0395 Homo | 2.5 |
| 425025 | AW953168 | Hs.12407 | ESTs | 2.5 |
| 416589 | AA652687 | Hs.96151 | Human DNA sequence from clone RP5-1103G7 | 2.5 |
| 404826 | | | Target Exon | 2.5 |
| 422938 | NM_001809 | Hs.1594 | centromere protein A (17 kD) | 2.5 |
| 421991 | NM_014918 | Hs.110488 | KIAA0990 protein | 2.5 |
| 417404 | NM_007350 | Hs.82101 | pleckstrin homology-like domain, family | 2.5 |
| 448516 | AW898595 | | gb: RC1-NN0073-260400-011-g09 NN0073 Homo | 2.5 |
| 403356 | NA | | ENSP00000251525*: Hypothetical protein KI | 2.5 |
| 404983 | | | ENSP00000252242*: Keratin, type II cytosk | 2.5 |
| 418282 | AA215535 | Hs.98133 | ESTs | 2.5 |
| 427409 | AW467143 | Hs.135411 | actin related protein | 2.5 |
| 431806 | AF186114 | Hs.270737 | tumor necrosis factor (ligand) superfami | 2.5 |
| 443367 | AW071349 | Hs.215937 | ESTs | 2.5 |
| 421246 | AW582962 | Hs.102897 | CGI-47 protein | 2.5 |
| 439217 | AF086041 | Hs.42975 | ESTs | 2.5 |
| 400925 | | | Target Exon | 2.5 |
| 404552 | NA | | ENSP00000220888*: ZINC FINGER TRANSCRIPTI | 2.5 |
| 417168 | AL133117 | Hs.81376 | Homo sapiens mRNA; cDNA DKFZp586L1121 (f | 2.5 |
| 418841 | NM_002332 | Hs.89137 | low density lipoprotein-related protein | 2.5 |
| 426853 | U32974 | Hs.172777 | baculoviral IAP repeat-containing 4 | 2.5 |
| 427738 | NM_000318 | Hs.180612 | peroxisomal membrane protein 3 (35 kD, Ze | 2.5 |
| 457384 | AA501760 | Hs.15806 | Homo sapiens mRNA; cDNA DKFZp434H2019 (f | 2.5 |
| 447128 | AI271898 | Hs.164866 | cyclin K | 2.5 |
| 454693 | AW813428 | | gb: MR3-ST0192-010200-210-c05 ST0192 Homo | 2.5 |
| 434657 | AA641876 | Hs.191840 | ESTs | 2.5 |
| 402077 | NA | | Target Exon | 2.5 |
| 400289 | X07820 | Hs.2258 | matrix metalloproteinase 10 (MMP10; str | 2.5 |
| 409723 | AW885757 | Hs.257862 | ESTs | 2.5 |
| 447020 | T27308 | Hs.16986 | hypothetical protein FLJ11046 | 2.5 |
| 455068 | AI807894 | Hs.47274 | Homo sapiens mRNA; cDNA DKFZp564B176 (fr | 2.5 |
| 431232 | AI024353 | Hs.131755 | hypothetical protein FLJ14298 | 2.5 |
| 408938 | AA059013 | Hs.22607 | ESTs | 2.5 |
| 411571 | AA122393 | Hs.70811 | hypothetical protein FLJ20516 | 2.5 |
| 426504 | AW162919 | Hs.170160 | RAB2, member RAS oncogene family-like | 2.5 |
| 428248 | AI126772 | Hs.40479 | ESTs | 2.5 |
| 408813 | AI580090 | Hs.48295 | RNA helicase family | 2.5 |
| 423504 | N80077 | Hs.24792 | chromosome 12 open reading frame 5 | 2.5 |
| 425441 | AA449644 | Hs.193063 | Homo sapiens cDNA FLJ14201 fis, clone NT | 2.5 |
| 443066 | AW297921 | Hs.255703 | ESTs | 2.5 |
| 443556 | AA256769 | Hs.94949 | methylmalonyl-CoA epimerase | 2.5 |
| 428943 | AW086180 | Hs.37636 | ESTs, Weakly similar to KIAA1392 protein | 2.5 |
| 425320 | U29344 | Hs.83190 | fatty acid synthase | 2.5 |
| 430388 | AA356923 | Hs.240770 | nuclear cap binding protein subunit 2, 2 | 2.5 |
| 423242 | AL039402 | Hs.125783 | DEME-6 protein | 2.5 |
| 416241 | N52639 | Hs.32683 | ESTs | 2.5 |
| 440244 | AI743977 | Hs.205144 | ESTs | 2.5 |
| 409239 | AA740875 | Hs.44307 | ESTs, Moderately similar to I38022 hypot | 2.5 |
| 452464 | AW500507 | Hs.192619 | KIAA1600 protein | 2.5 |
| 410718 | AI920783 | Hs.191435 | ESTs | 2.5 |
| 408877 | AA479033 | Hs.130315 | ESTs, Weakly similar to A47582 B-cell gr | 2.5 |
| 445150 | AI446747 | Hs.338704 | olfactory receptor, family 7, subfamily | 2.5 |
| 407756 | AA116021 | Hs.38260 | ubiquitin specific protease 18 | 2.5 |
| 407633 | NM_007069 | Hs.37189 | similar to rat HREV107 | 2.5 |
| 449754 | H00820 | Hs.30977 | ESTs, Weakly similar to B34087 hypotheti | 2.5 |
| 419316 | AA236255 | Hs.298419 | ESTs | 2.5 |
| 429118 | H20669 | Hs.35406 | ESTs, Highly similar to unnamed protein | 2.5 |
| 440331 | AL046412 | Hs.202151 | ESTs | 2.5 |
| 449344 | AI640355 | Hs.312691 | ESTs | 2.5 |
| 459006 | AW298631 | Hs.27721 | Wolf-Hirschhorn syndrome candidate 1-lik | 2.5 |
| 423165 | AI937547 | Hs.124915 | hypothetical protein MGC2601 | 2.5 |
| 411337 | AW837349 | | gb: QV2-LT0038-270300-108-d12 LT0038 Homo | 2.5 |
| 438290 | AA843719 | Hs.122341 | ESTs | 2.5 |
| 406414 | | | C5000506*: gi\|124941\|sp\|P18614\|ITA1_RAT I | 2.5 |
| 424498 | AB033043 | Hs.149377 | hypothetical protein DKFZp761L0424 | 2.5 |
| 443464 | BE548446 | Hs.5167 | Homo sapiens mRNA; cDNA DKFZp434F152 (fr | 2.5 |
| 424856 | AA347746 | Hs.9521 | ESTs, Weakly similar to ZN43_HUMAN ZINC | 2.5 |
| 440304 | BE159984 | Hs.125395 | ESTs | 2.5 |
| 409045 | AA635062 | Hs.50094 | Homo sapiens mRNA; cDNA DKFZp434O0515 (f | 2.5 |
| 422648 | D86983 | Hs.118893 | Melanoma associated gene | 2.5 |
| 428819 | AL135623 | Hs.193914 | KIAA0575 gene product | 2.5 |
| 412520 | AA442324 | Hs.795 | H2A histone family, member O | 2.5 |
| 430602 | D13752 | Hs.184927 | cytochrome P450, subfamily XIB (steroid | 2.5 |
| 408031 | AA081395 | Hs.42173 | Homo sapiens cDNA FLJ10366 fis, clone NT | 2.5 |
| 403133 | | | Target Exon | 2.5 |
| 413189 | BE070231 | | gb: QV4-BT0407-260100-087-f12 BT0407 Homo | 2.5 |
| 400346 | AB041269 | Hs.272263 | Homo sapiens mRNA for keratin 19, partia | 2.5 |
| 435509 | AI458679 | Hs.181915 | ESTs | 2.5 |
| 458145 | AI239457 | Hs.130794 | ESTs | 2.5 |
| Table 19 shows 1045 genes up-regulated in breast cancer compared to normal adult tissues. These were selected from 59680 probesets on the Affymetrix/Eos-Hu03 GeneChip array such that the ratio of “average” breast cancer to “average” normal adult tissues was greater than or equal to 2.5. The “average” breast cancer level was set to | | | | |
| #the 90^{th} percentile value. The “average” normal adult tissue level was set to the 90^{th} percentile value amongst 144 non-malignant tissues. In order to remove gene-specific background levels of non-specific hybridization, the 15^{th} percentile value amongst the 144 non-malignant tissues was subtracted from both the numerator and the denominator before the ratio was evaluated. | | | | |

Pkey: Unique Eos probeset identifier number
ExAccn: Exemplar Accession number, Genbank accession number
UnigeneID: Unigene number
Unigene Title: Unigene gene title
R1: Ratio of tumor to normal body tissue

TABLE 19A
Table 19A shows the accession numbers for those pkeys lacking unigeneID's for Table 19.
For each probeset, we have listed the gene cluster number from which the oligonucleotides
were designed. Gene clusters were compiled using sequences derived from Genbank ESTs
and mRNAs. These sequences were clustered based on sequence similarity using Clustering
and Alignment Tools (DoubleTwist, Oakland California). The Genbank accession numbers
for sequences comprising each cluster are listed in the “Accession” column.

| Pkey | CAT number | Accessions |
|---|---|---|
| 407647 | 1007366_1 | AW860158 AW862385 AW860159 AW862386 AW862341 AW821869 AW821893 AW062660 AW062656 |
| 407980 | 103087_1 | AA046309 AI263500 AA046397 |
| 408254 | 1049346_1 | AW807227 AW807576 AW807137 AW807157 AW807495 AW807494 AW807417 AW807083 AW845786 AW845801 |
| | | AW807130 AW807335 AW807081 AW807349 AW807339 AW807164 AW807341 AW807224 AW845903 AW177424 |
| | | AW807159 AW807123 BE141576 AW807340 AW807334 AW807520 AW807205 AW807505 BE141574 AW807390 |
| | | AW807395 AW845789 AW807101 AW807089 AW807519 AW807239 AW807509 AW807356 AW807526 AW807098 |
| | | AW807307 AW807153 AW807295 AW807313 AW807322 AW807265 AW807513 AW807516 AW807166 AW807501 |
| | | AW807120 AW807168 AW807121 AW807527 AW807151 AW807203 AW807489 AW807511 AW807158 AW845800 |
| | | AW807507 AW845793 AW845796 AW807296 AW845781 AW807444 AW845871 AW807512 AW807242 AW807141 |
| | | AW807522 AW807487 AW807514 AW807142 AW807232 AW807379 AW807114 AW807518 AW807199 AW807211 |
| | | AW807498 AW807086 AW807492 AW807218 AW807082 AW807525 AW807493 AW807523 AW807087 AW845784 |
| | | AW807037 AW807128 AW807080 AW807118 AW845807 AW807524 AW845803 AW807249 AW845795 AW807160 |
| | | AW807343 AW807515 AW807233 AW807289 AW177102 AW807352 AW807394 AW177105 AW807176 AW177103 |
| | | AW845870 AW177099 AW177101 AW807528 AW807336 AW807038 AW177100 AW807411 AW807088 AW845865 |
| | | AW807226 AW807517 AW807397 AW807303 AW807177 AW807154 AW807136 AW807146 AW807085 AW807521 |
| | | AW807488 AW807385 AW807355 AW807223 AW807155 |
| 409163 | 110418_1 | AA065081 AA075017 AA084791 AA071015 AA081560 AA071459 AA545727 AA083100 AA085366 AA115845 AA075457 |
| | | AA064704 AA082878 AA075742 AA069162 |
| 409695 | 114876_1 | AA296961 AA296889 AA076945 AA077528 AA077497 |
| 410534 | 1207247_1 | AW905138 AW753008 R13818 Z43519 |
| 410672 | 1214882_1 | AW794600 AW794730 |
| 410784 | 1221005_1 | AW803201 BE079700 BE062940 |
| 410785 | 1221055_1 | AW803341 AW803265 AW803403 AW803466 AW803402 AW803413 AW803268 AW803396 AW803334 AW803355 |
| 410835 | 1223785_1 | AW806906 AW806915 AW866460 AW866475 AW866462 AW866448 AW866372 AW866604 |
| 411050 | 1230330_1 | AW814902 BE156656 BE156667 BE156590 BE156441 BE156447 |
| 411086 | 1231500_1 | BE070800 AW875226 BE149115 |
| 411093 | 1231970_1 | BE067650 AW817053 |
| 411111 | 1232669_1 | AW818127 AW818161 R09719 |
| 411171 | 1234393_1 | AW820260 AW820332 R94406 |
| 411337 | 1239217_1 | AW837349 AW837355 AW882717 |
| 411514 | 1248638_1 | AW850178 AW850233 AW850445 AW850446 |
| 411670 | 1253680_1 | AW856552 AW861101 AW856574 AW861099 AW861100 AW856573 AW856576 AW856562 |
| 411905 | 1265181_1 | BE265067 BE264978 AW875420 |
| 412102 | 1277395_1 | H56435 H56572 AW892929 |
| 412209 | 1283610_1 | AW901456 AW901450 AW901441 |
| 412248 | 1285000_1 | BE176480 AW903298 AW903313 |
| 413043 | 1346556_1 | BE158766 BE061699 BE147360 BE147362 BE061666 BE061697 BE061647 BE061678 |
| 413111 | 1349546_1 | BE065837 BE065805 BE065799 BE065818 BE065839 BE065831 BE065894 BE065789 BE065792 |
| 413189 | 1352723_1 | BE070231 BE070229 BE070255 |
| 413221 | 1353887_1 | BE161151 BE162495 BE161002 BE072205 BE160989 BE162482 |
| 413499 | 1373910_1 | BE144884 H97942 |
| 413708 | 1384140_1 | BE158791 BE158806 BE158748 BE158744 BE158740 BE158739 BE158811 BE158700 BE158741 BE158683 BE158685 |
| 414210 | 1426051_1 | BE383592 BE261671 |
| 414596 | 1465004_1 | BE386870 Z41986 H08501 |
| 414605 | 1465790_−1 | BE390440 |
| 415747 | 155189_1 | AA381209 AA381245 AA167683 |
| 416173 | 1574973_1 | R52782 R17313 H24192 R19876 |
| 417742 | 1696282_1 | R64719 Z44680 R12451 |
| 417974 | 171237_1 | AA210765 T95700 H94407 |
| 418636 | 177402_1 | AW749855 AA225995 AW750208 AW750206 |
| 419536 | 185688_1 | AA603305 AA244095 AA244183 |
| 420854 | 197072_1 | AW296927 AI684514 AI263168 AA281079 |
| 422156 | 212379_1 | N34524 AA305071 AW954803 AA502335 AI433430 AI203597 AW026670 AW265323 AW850787 AA317554 AW993643 |
| | | AW835572 AW385512 AI334966 W32951 H62656 H53902 R88904 AW835732 |
| 422996 | 223666_1 | BE091089 BE091123 AA319959 |
| 423833 | 232451_1 | AW503329 N46610 AA331571 |
| 423841 | 232507_1 | AW753967 AA370795 AA331630 AW962550 |
| 423945 | 233566_1 | AA410943 AW948953 AA334202 AA332882 |
| 425201 | 247933_1 | AA352111 AW962247 AA429695 |
| 426650 | 270283_1 | AA382814 AA402411 AA412355 |
| 426878 | 273265_1 | BE069341 AW748403 AL044891 AI908240 AA393080 |
| 430264 | 315008_1 | AA470519 BE303010 BE302954 BE384120 |
| 430785 | 323486_1 | Z30201 AA486132 T72025 |
| 431676 | 336411_1 | AI685464 AW971336 AA513587 AA525142 |
| 433687 | 373061_1 | AA743991 AA604852 AW272737 |
| 434338 | 383982_1 | AW754311 AA630185 AW803285 |
| 434469 | 387447_1 | AA634806 C18732 AA729161 AA729860 |
| 435447 | 406400_1 | AI872932 AA682306 BE220163 W88695 T81307 H91447 |
| 437152 | 43386_1 | AL050027 BE089051 |
| 437854 | 44418_1 | AL119723 AL119874 AI909018 U50537 |
| 439031 | 46798_1 | AF075079 H48601 H48795 |
| 439255 | 470321_1 | BE164500 AA832198 BE164502 |
| 444910 | 624951_1 | AI201849 BE069007 AW946544 |
| 445432 | 63943_1 | AV653771 BE089370 |
| 446922 | 69865_1 | BE175605 Z43529 F06610 BE175602 AV661027 |
| 447197 | 711623_1 | R36075 AI366546 R36167 |
| 448420 | 76273_1 | BE623004 AA380669 BE263627 BE246433 |
| 448516 | 766241_1 | AW898595 AW898588 AW898590 AW898663 AW898592 AI525093 |
| 450522 | 837264_1 | AI698839 AI909260 AI909259 |
| 450736 | 844652_1 | AW970060 AI732366 AI792313 AW839644 |
| 451024 | 85565_1 | AA442176 AA259181 |
| 451067 | 85759_1 | BE172186 AA059279 AA020815 AA013437 |
| 451340 | 86640_1 | AW936273 AW340350 AA017208 |
| 452542 | 921410_1 | AW812256 AW812257 AI906423 AI906422 |
| 452564 | 92227_1 | AA026777 N50065 R09961 N54721 |
| 453472 | 968371_1 | AL037925 AL037931 AL037957 |
| 454307 | 1106070_1 | AW855717 AW362452 AW362443 |
| 454359 | 1130674_1 | N71277 AW390764 |
| 454545 | 1223779_1 | AW806899 AW866451 AW866393 AW866297 AW817869 |
| 454693 | 1229132_1 | AW813428 AW813444 AW813367 AW813368 AW813429 AW813424 |
| 454714 | 1230493_1 | AW815098 BE154843 BE154831 |
| 455047 | 1250536_1 | AW852530 AW852527 AW852526 |
| 455092 | 1252971_1 | BE152428 AW855572 AW855607 |
| 455097 | 1253130_1 | AW855802 AW855794 AW855797 AW855806 AW855796 AW855808 AW855793 AW855807 |
| 455100 | 1253334_1 | BE160198 AW935898 T11520 AW935930 AW856073 AW861034 |
| 455431 | 1289854_1 | AW938484 BE001245 BE001190 |
| 455511 | 1321229_1 | BE144762 AW979091 |
| 455609 | 1337548_1 | BE011668 BE011689 BE011627 BE011679 BE011699 BE011678 BE011696 BE011675 BE011622 BE011635 |
| 455651 | 1348732_1 | BE064962 BE064979 BE064853 BE064857 BE064856 BE064977 BE064960 BE064860 BE064815 BE064957 BE064804 |
| | | BE064816 BE064850 BE064806 BE064796 BE064818 BE064975 BE064819 BE064810 BE064668 BE065059 |
| 455685 | 1350393_1 | BE066976 BE066928 BE066927 |
| 455700 | 1351264_1 | BE068115 BE068104 BE068102 BE068096 BE068103 BE068154 BE068198 |
| 455708 | 1352232_1 | BE069326 BE069290 BE069352 |
| 455732 | 1353874_1 | BE080908 BE072258 BE072190 BE072236 |
| 455838 | 1374605_1 | BE145808 BE145807 BE181883 |
| 455935 | 1384144_1 | BE158687 BE158688 |
| 455945 | 1385588_1 | BE160636 BE160606 BE160703 |
| 456207 | 165078_−1 | AA193450 |
| 456482 | 192289_1 | AA485224 AA287308 AA258121 |
| 458094 | 47311_1 | AF086325 W72956 W73221 AA219112 |
| 458673 | 679507_1 | N99626 AI302701 |

Pkey: Unique Eos probeset identifier number
CAT number: Gene cluster number
Accession: Genbank accession numbers

TABLE 19B
Table 19B shows the genomic positioning for those pkeys lacking unigene ID's
and accession numbers in Table 19. For each predicted exon, we have listed
the genomic sequence source used for prediction. Nucleotide locations of
each predicted exon are also listed.

| Pkey | Ref | Strand | Nt_position |
|---|---|---|---|
| 400555 | 9801191 | Minus | 134694-134817 |
| 400608 | 9887666 | Minus | 96756-97558 |
| 400610 | 9887671 | Minus | 117606-117928, 124040-124147 |
| 400925 | 7651921 | Plus | 38183-38391, 43900-44086 |
| 401045 | 8117619 | Plus | 90044-90184, 91111-91345 |
| 401049 | 7232177 | Plus | 149157-150692 |
| 401093 | 8516137 | Minus | 22335-23166 |
| 401256 | 9796573 | Minus | 45482-45620 |
| 401283 | 9800093 | Minus | 47256-47456 |
| 401326 | 9212516 | Minus | 226246-227505 |
| 401418 | 7452889 | Minus | 124865-125075 |
| 401451 | 6634068 | Minus | 119926-121272 |
| 401458 | 9187886 | Plus | 76485-77597 |
| 401497 | 7381770 | Plus | 92607-92813 |
| 401508 | 7534110 | Minus | 110779-110983 |
| 401575 | 7229804 | Minus | 76253-76364 |
| 401747 | 9789672 | Minus | 118596-118816, 119119-119244, 119609-119761, |
| | | | 120422-120990, 130161-130381, 130468-130593, 131097-131258, |
| | | | 131866-131932, 132451-132575, 133580-134011 |
| 401781 | 7249190 | Minus | 83215-83435, 83531-83656, 83740-83901, |
| | | | 84237-84393, 84955-85037, 86290-86814 |
| 401785 | 7249190 | Minus | 165776-165996, 166189-166314, 166408-166569, |
| | | | 167112-167268, 167387-167469, 168634-168942 |
| 401793 | 7263888 | Minus | 102945-103083 |
| 401987 | 4406829 | Minus | 72893-73021, 76938-77049 |
| 402077 | 8117414 | Plus | 65014-65195 |
| 402109 | 8131678 | Minus | 171722-171859, 173197-173303 |
| 402184 | 8576001 | Minus | 112844-112986, 113505-113636 |
| 402376 | 9625329 | Minus | 21753-22385 |
| 402421 | 9796341 | Minus | 46609-46662, 46758-46811, 86293-86346, 89776-89829, |
| | | | 90048-90101, 102817-102924 |
| 402578 | 9884928 | Plus | 66350-66496 |
| 402606 | 9909429 | Minus | 81747-82094 |
| 402696 | 7328818 | Minus | 23600-23731 |
| 402765 | 9367757 | Plus | 109588-109726 |
| 402800 | 6010175 | Plus | 43921-44049, 46181-46273 |
| 402820 | 6456853 | Minus | 82274-82443 |
| 402892 | 8086844 | Minus | 194384-194645 |
| 403133 | 7331427 | Plus | 38314-38634 |
| 403356 | 8669930 | Plus | 92839-93036 |
| 403388 | 9438331 | Plus | 112733-113001, 114599-114735 |
| 403426 | 9719529 | Minus | 157156-158183 |
| 403585 | 8101208 | Minus | 131266-131769 |
| 403593 | 6862650 | Minus | 62554-62712, 69449-69602 |
| 403637 | 8671936 | Minus | 142647-142771, 145531-145762 |
| 403639 | 8671948 | Plus | 113234-113326, 115186-115287, 119649-119786 |
| 403677 | 7331517 | Minus | 55008-55083, 62860-63051 |
| 403775 | 7770580 | Minus | 102247-102326, 103095-103148 |
| 403943 | 7711864 | Plus | 100742-100904, 101322-101503 |
| 404091 | 7684554 | Minus | 82121-83229 |
| 404097 | 7770701 | Plus | 55512-55781 |
| 404142 | 9856692 | Minus | 80316-80459 |
| 404253 | 9367202 | Minus | 55675-56055 |
| 404274 | 9885189 | Plus | 104127-104318 |
| 404285 | 2326514 | Plus | 32282-32416 |
| 404360 | 9858450 | Minus | 122873-122966, 151324-151469, 153093-153253 |
| 404440 | 7528051 | Plus | 80430-81581 |
| 404443 | 7579073 | Minus | 87198-87441 |
| 404552 | 7243881 | Plus | 19854-20010 |
| 404561 | 9795980 | Minus | 69039-70100 |
| 404580 | 6539738 | Minus | 240588-241589 |
| 404721 | 9856648 | Minus | 173763-174294 |
| 404826 | 6572184 | Plus | 47726-48046 |
| 404983 | 4432779 | Minus | 51178-51374, 52000-52173 |
| 405037 | 7543748 | Minus | 127374-127578 |
| 405041 | 7547195 | Plus | 121230-121714 |
| 405095 | 8072599 | Plus | 138877-139066 |
| 405153 | 9965565 | Minus | 175317-175500 |
| 405196 | 7230083 | Minus | 135716-135851 |
| 405232 | 7249042 | Plus | 125904-126063 |
| 405248 | 7259728 | Plus | 637-777 |
| 405336 | 6094635 | Plus | 33267-33563 |
| 405394 | 6624123 | Minus | 31900-32373 |
| 405460 | 7684569 | Minus | 52223-52389 |
| 405494 | 8050952 | Minus | 70284-70518 |
| 405547 | 1054740 | Plus | 124361-124520, 124914-125050 |
| 405609 | 5757553 | Minus | 42814-43010, 43583-43783, 44863-45033, |
| | | | 46429-46554, 47815-48018, 49961-50153, |
| | | | 51624-51727, 51823-51959, 52702-52918, |
| | | | 55469-55601, 57111-57307, 58169-58296, |
| | | | 60215-60332, 61482-61727 |
| 405638 | 6289229 | Plus | 199260-199372, 199826-199929 |
| 405654 | 4895155 | Minus | 53624-53759 |
| 405718 | 9795467 | Plus | 113080-113266 |
| 405822 | 6273498 | Minus | 154660-154974, 155203-155379 |
| 405848 | 7651809 | Minus | 28135-28244 |
| 405873 | 6758747 | Minus | 32129-32764 |
| 405906 | 7705124 | Minus | 10835-11059 |
| 405917 | 7712162 | Minus | 106829-107213 |
| 405925 | 6758795 | Plus | 129935-130282 |
| 405953 | 7960374 | Minus | 65101-65574 |
| 406069 | 9117732 | Plus | 68880-69374 |
| 406151 | 7144806 | Minus | 94087-94285 |
| 406153 | 9929734 | Minus | 12902-13069 |
| 406182 | 5923650 | Minus | 28256-28935 |
| 406271 | 7534217 | Plus | 36179-36692 |
| 406291 | 5686274 | Plus | 9562-9867 |
| 406340 | 9255985 | Minus | 71754-71944 |
| 406414 | 9256407 | Plus | 49593-49850 |
| 406446 | 9454509 | Minus | 116424-116527, 118721-118859, 121187-121364 |
| 406504 | 7711360 | Minus | 107068-107277 |
| 406554 | 7711566 | Plus | 106956-107121 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 20
544 GENES UP-REGULATED IN BREAST CANCER
COMPARED TO NORMAL ADULT TISSUES THAT ARE
LIKELY TO ENCODE EXTRACELLULAR OR CELL-
SURFACE PROTEINS

| Pkey: | ExAccn | UnigeneID | Pred. Prot. Domains | UnigeneTitle | R1 |
|---|---|---|---|---|---|
| 408591 | AF015224 | Hs.46452 | SS, Uteroglobin, SS, Uteroglobin | mammaglobin 1 | 168.6 |
| 400291 | AA401369 | Hs.190721 | TM | ESTs | 73.2 |
| 449746 | AI668594 | Hs.176588 | , SS, p450 | ESTs, Weakly similar to CP4Y_HUMAN CYTOC | 65.7 |
| 407277 | AW170035 | Hs.326736 | TM | Homo sapiens breast cancer antigen NY-BR | 57.6 |
| 400292 | AA250737 | Hs.72472 | death, ZU5, TM, Activin_recp, pkinase, | BMP-R1B | 55.9 |
| 424735 | U31875 | Hs.272499 | , SS, TM | short-chain alcohol dehydrogenase family | 53.8 |
| 426878 | BE069341 | | TM | gb: QV3-BT0381-270100-073-c08 BT0381 Homo | 50.3 |
| 428848 | NM_000230 | Hs.194236 | SS, Leptin, SS, Leptin, | leptin (murine obesity homolog) | 40.8 |
| 407178 | AA195651 | Hs.104106 | , SS, Dihydroorotase, | ESTs | 39.3 |
| 408000 | L11690 | Hs.620 | Plectin_repeat, SH3, spectrin, SS, Plectin_r | bullous pemphigoid antigen 1 (230/240 kD) | 37.3 |
| 427585 | D31152 | Hs.179729 | SS, C1q, Collagen, SS, C1q, Collagen, | collagen, type X, alpha 1 (Schmid metaph | 35.2 |
| 429441 | AJ224172 | Hs.204096 | , SS, Uteroglobin, | lipophilin B (uteroglobin family member) | 30.0 |
| 450375 | AA009647 | Hs.8850 | , SS, TM, disintegrin, Pep_M12B_propep, Repro | a disintegrin and metalloproteinase doma | 25.7 |
| 420931 | AF044197 | Hs.100431 | SS, IL8, SS | small inducible cytokine B subfamily (Cy | 25.2 |
| 422109 | S73265 | Hs.1473 | SS, Bombesin, SS | gastrin-releasing peptide | 24.8 |
| 445730 | AI624342 | Hs.170042 | , SS, TM, Cation_efflux | ESTs | 24.1 |
| 451110 | AI955040 | Hs.265398 | SS | ESTs, Weakly similar to transformation-r | 24.0 |
| 400297 | AI127076 | Hs.334473 | TM | hypothetical protein DKFZp564O1278 | 23.8 |
| 420813 | X51501 | Hs.99949 | SS, SS | prolactin-induced protein | 22.8 |
| 452744 | AI267652 | Hs.30504 | , SS, TM, GNS1_SUR4, cNMP_binding, RIIa | Homo sapiens mRNA; cDNA DKFZp434E082 (fr | 22.6 |
| 424634 | NM_003613 | Hs.151407 | ig, tsp_1, SS, AAA | cartilage intermediate layer protein, nu | 21.7 |
| 420757 | X78592 | Hs.99915 | hormone_rec, Androgen_recep, zf-C4, | androgen receptor (dihydrotestosterone r | 21.7 |
| 424399 | AI905687 | Hs.2533 | SS | aldehyde dehydrogenase 9 family, member | 20.3 |
| 447350 | AI375572 | Hs.172634 | , pkinase, | ESTs | 19.2 |
| 456207 | AA193450 | | , SS, TM, p450, p450 | gb: zr40e07.r1 Soares_NhHMPu_S1 Homo sapi | 18.3 |
| 431448 | AL137517 | Hs.334473 | TM | hypothetical protein DKFZp564O1278 | 18.2 |
| 427217 | AA399272 | Hs.144341 | SS | ESTs | 18.2 |
| 456938 | X52509 | Hs.161640 | , SS, TM, aminotran_1_2, Cadherin_C_term, cad | tyrosine aminotransferase | 18.1 |
| 435496 | AW840171 | Hs.265398 | SS | ESTs, Weakly similar to transformation-r | 17.9 |
| 402578 | | | SS, p450, SS, TM, p450 | C1001134: gi\|2117372\|pir\|\|I65981 fatty ac | 17.8 |
| 453160 | AI263307 | Hs.239884 | SS | H2B histone family, member L | 17.8 |
| 422505 | AL120862 | Hs.124165 | SS | programmed cell death 9 (PDCD9) | 17.7 |
| 444342 | NM_014398 | Hs.10887 | Lamp, SS, TM, Lamp, | similar to lysosome-associated membrane | 17.5 |
| 449765 | N92293 | Hs.206832 | SS | ESTs, Moderately similar to ALU8_HUMAN A | 17.3 |
| 428227 | AA321649 | Hs.2248 | SS, IL8, | small inducible cytokine subfamily B (CX | 17.0 |
| 425692 | D90041 | Hs.155956 | , SS, Acetyltransf2, | N-acetyltransferase 1 (arylamine N-acety | 16.7 |
| 424001 | W67883 | Hs.137476 | , pkinase, | paternally expressed 10 | 16.5 |
| 448595 | AB014544 | Hs.21572 | LRRCT, LRR, SS, LRRCT, serine_carbpept | KIAA0644 gene product | 16.3 |
| 449448 | D60730 | Hs.57471 | SS | ESTs | 16.2 |
| 418007 | M13509 | Hs.83169 | SS, hemopexin, Peptidase_M10, SS, Peptidase_{—} | matrix metalloproteinase 1 (MMP1; inters | 15.7 |
| 418994 | AA296520 | Hs.89546 | SS, lectin_c, sushi, EGF, SS, EGF, lectin_c, su | selectin E (endothelial adhesion molecul | 15.5 |
| 453596 | AA441838 | Hs.62905 | SS | hypothetical protein FLJ14834 | 15.5 |
| 452401 | NM_007115 | Hs.29352 | , SS, CUB, Xlink, | tumor necrosis factor, alpha-induced pro | 15.0 |
| 446591 | H44186 | Hs.15456 | PDZ, SS | PDZ domain containing 1 | 14.9 |
| 419296 | AA236115 | Hs.120785 | SS | ESTs | 14.8 |
| 452838 | U65011 | Hs.30743 | SS, SS | preferentially expressed antigen in mela | 14.7 |
| 422805 | AA436989 | Hs.121017 | histone, SS, histone, histone | H2A histone family, member A | 14.3 |
| 448390 | AL035414 | Hs.21068 | SS | hypothetical protein | 14.2 |
| 447342 | AI199268 | Hs.19322 | , SS, lipocalin | Homo sapiens, Similar to RIKEN cDNA 2010 | 14.2 |
| 411869 | W20027 | Hs.23439 | , SS, Peptidase_M1, | ESTs | 13.9 |
| 443709 | AI082692 | Hs.134662 | , SS, TM, SNF | ESTs | 13.7 |
| 459587 | AA031956 | | , SS, LIM, | gb: zk15e04.s1 Soares_pregnant_uterus_NbH | 13.7 |
| 442580 | AI733682 | Hs.130239 | SS | ESTs | 13.5 |
| 400289 | X07820 | Hs.2258 | hemopexin, Peptidase_M10, SS, Peptidase_M10 | matrix metalloproteinase 10 (MMP10; str | 13.5 |
| 411598 | BE336654 | Hs.70937 | histone, SS, histone, histone | H3 histone family, member A | 13.3 |
| 415263 | AA948033 | Hs.130853 | , SS, histone, histone, linker_histone | ESTs | 13.2 |
| 433805 | AA706910 | Hs.112742 | , SS, Ribosomal_L7Ae, | ESTs | 13.1 |
| 407276 | AI951118 | Hs.326736 | TM | Homo sapiens breast cancer antigen NY-BR | 13.1 |
| 443348 | AW873596 | Hs.182278 | , SS, DENN | calmodulin 2 (phosphorylase kinase, delt | 13.0 |
| 421037 | AI684808 | Hs.197653 | SS | programmed cell death 9 (PDCD9) | 12.9 |
| 424086 | AI351010 | Hs.102267 | , SS, Lysyl_oxidase | lysyl oxidase | 12.8 |
| 400295 | W72838 | Hs.2533 | SS | aldehyde dehydrogenase 9 family, member | 12.7 |
| 452461 | N78223 | Hs.108106 | , SS, G9a, PHD, | transcription factor | 12.5 |
| 427365 | AI873274 | Hs.190721 | TM | ESTs | 12.4 |
| 433365 | AF026944 | Hs.293797 | , SS, TPR | ESTs | 12.3 |
| 409269 | AA576953 | Hs.22972 | SS, TM, UPF0016, SS, TM, UPF0016 | hypothetical protein FLJ13352 | 12.0 |
| 432596 | AJ224741 | Hs.278461 | SS, EGF, vwa, SS, TM, vwa, | matrilin 3 | 11.9 |
| 408771 | AW732573 | Hs.47584 | TM, K_tetra, ion_trans, SS, TM, K_tetra, ion_t | potassium voltage-gated channel, delayed | 11.9 |
| 432912 | BE007371 | Hs.200313 | , SS, TM, Folate_carrier | ESTs | 11.9 |
| 447033 | AI357412 | Hs.157601 | SS | ESTs | 11.8 |
| 421155 | H87879 | Hs.102267 | SS, Lysyl_oxidase, Aldose_epim, Epimerase, S | lysyl oxidase | 11.8 |
| 424905 | NM_002497 | Hs.153704 | pkinase, SS, TM, pkinase, polyprenyl_synt, | NIMA (never in mitosis gene a)-related k | 11.7 |
| 425398 | AL049689 | Hs.156369 | SS | hypothetical protein similar to tenascin | 11.6 |
| 438167 | R28363 | Hs.24286 | , SS, TM, 7tm_1, p450, rrm | ESTs | 11.5 |
| 459583 | AI907673 | | , pkinase, | gb: IL-BT152-080399-004 BT152 Homo sapien | 11.5 |
| 423945 | AA410943 | | death, ZU5, TM, Activin_recp, pkinase, | gb: zt32h03.r1 Soares ovary tumor NbHOT H | 11.4 |
| 439820 | AL360204 | Hs.283853 | SS | Homo sapiens mRNA full length insert cDN | 11.4 |
| 402606 | | | SS | NM_024626: Homo sapiens hypothetical prot | 11.3 |
| 445263 | H57646 | Hs.42586 | , SS, Acyltransferase, | KIAA1560 protein | 11.2 |
| 430217 | N47863 | Hs.336901 | , SS, RNA_pol_A, RNA_pol_A2, | e, ribosomal protein S24 | 11.1 |
| | | | Ribosomal_S24 | | |
| 447164 | AF026941 | Hs.17518 | , TM, IBR | Homo sapiens cig5 mRNA, partial sequence | 11.1 |
| 431385 | BE178536 | Hs.11090 | , SS, TM | membrane-spanning 4-domains, subfamily A | 11.1 |
| 423887 | AL080207 | Hs.134585 | , SS, TM, BRCT, ank, ABC_tran, ABC_tran | DKFZP434G232 protein | 10.9 |
| 415385 | R17798 | Hs.7535 | , SS, Fork_head, | COBW-like protein | 10.9 |
| 425704 | U79293 | Hs.159264 | SS | Human clone 23948 mRNA sequence | 10.7 |
| 429859 | NM_007050 | Hs.225952 | , SS, TM, Y_phosphatase, MAM, fn3, | protein tyrosine phosphatase, receptor t | 10.4 |
| 425523 | AB007948 | Hs.158244 | , SS, laminin_B, laminin_EGF, laminin_Nterm | KIAA0479 protein | 10.3 |
| 428368 | BE440042 | Hs.83326 | SS, Peptidase_M10, hemopexin, SS, Peptidase_{—} | matrix metalloproteinase 3 (stromelysin | 10.3 |
| 418912 | NM_000685 | Hs.89472 | SS, TM, 7tm_1, SS, TM, 7tm_1, | angiotensin receptor 1 | 10.3 |
| 422026 | U80736 | Hs.110826 | SS | trinucleotide repeat containing 9 | 10.3 |
| 451952 | AL120173 | Hs.301663 | , SS, pkinase, | ESTs | 10.3 |
| 438199 | AW016531 | Hs.122147 | , SS, ArfGap, | ESTs | 10.2 |
| 400608 | | | SS, TM, SS, TM | C10001899: gi\|7508633\|pir\|\|T25392 hypothe | 10.1 |
| 413472 | BE242870 | Hs.75379 | SS | solute carrier family 1 (glial high affi | 10.0 |
| 432374 | W68815 | Hs.301885 | SS | Homo sapiens cDNA FLJ11346 fis, clone PL | 9.9 |
| 402408 | NA | | , SS, carb_anhydrase | NM_030920*: Homo sapiens hypothetical pro | 9.8 |
| 445537 | AJ245671 | Hs.12844 | , SS, TM, ras | EGF-like-domain, multiple 6 (EGFL6) | 9.7 |
| 451621 | AI879148 | Hs.26770 | SS, lipocalin, lipocalin, | fatty acid binding protein 7, brain | 9.6 |
| 405654 | NA | | BTB, SS | C12001521: gi\|7513934\|pir\|\|T31081 cca3 pr | 9.6 |
| 434988 | AI418055 | Hs.161160 | SS | ESTs | 9.6 |
| 416220 | N49776 | Hs.170994 | , SS, TM | hypothetical protein MGC10946 | 9.5 |
| 431808 | M30703 | Hs.270833 | SS, TM, EGF, SS | amphiregulin (schwannoma-derived growth | 9.5 |
| 414142 | AW368397 | Hs.150042 | , SS, UDPGT | Homo sapiens cDNA FLJ14438 fis, clone HE | 9.4 |
| 400298 | AA032279 | Hs.61635 | TM | six transmembrane epithelial antigen of | 9.4 |
| 418601 | AA279490 | Hs.86368 | SS, TM, calreticulin, SS, TM, calreticulin, | calmegin | 9.4 |
| 415539 | AI733881 | Hs.72472 | death, ZU5, TM, Activin_recp, pkinase, | BMP-R1B | 9.4 |
| 421451 | AA291377 | Hs.50831 | TM | ESTs | 9.3 |
| 429432 | AI678059 | Hs.202676 | SS | synaptonemal complex protein 2 | 9.3 |
| 442441 | AI820662 | Hs.129598 | SS | ESTs | 9.1 |
| 426429 | X73114 | Hs.169849 | , SS, TM, fn3, ig, | myosin-binding protein C, slow-type | 9.1 |
| 406687 | M31126 | Hs.272620 | SS, Peptidase_M10, hemopexin, SS, Peptidase_{—} | matrix metalloproteinase 11 (MMP11; stro | 9.1 |
| 448693 | AW004854 | Hs.228320 | SS | hypothetical protein FLJ23537 | 9.1 |
| 419948 | AB041035 | Hs.93847 | Ferric_reduct, TM, Ferric_reduct, | NM_016931: Homo sapiens NADPH oxidase 4 ( | 9.1 |
| 426214 | H59846 | Hs.128355 | SS | ESTs, Moderately similar to ALU7_HUMAN A | 9.0 |
| 427718 | AI798680 | Hs.25933 | , SS, TM, histone, Sec1, histone, sugar_tr | ESTs | 8.9 |
| 414812 | X72755 | Hs.77367 | SS, IL8, SS, IL8 | monokine induced by gamma interferon | 8.8 |
| 400285 | NA | | , TM, ABC_tran, ABC_membrane, | Eos Control | 8.8 |
| 422330 | D30783 | Hs.115263 | SS, TM, EGF, SS, TM | epiregulin | 8.8 |
| 416182 | NM_004354 | Hs.79069 | cyclin, SS | cyclin G2 | 8.8 |
| 420077 | AW512260 | Hs.87767 | SS | ESTs | 8.7 |
| 452281 | T93500 | Hs.28792 | , SS, TGF-beta, TGFb_propeptide, | Homo sapiens cDNA FLJ11041 fis, clone PL | 8.7 |
| 434531 | AA642007 | Hs.116369 | SS | ESTs | 8.6 |
| 408380 | AF123050 | Hs.44532 | , SS, TM, ubiquitin, 7tm_3, ANF_receptor, sush | diubiquitin | 8.6 |
| 443788 | AI732643 | Hs.144151 | TM | ESTs | 8.6 |
| 411078 | AI222020 | Hs.182364 | SS, SS | CocoaCrisp | 8.5 |
| 445495 | BE622641 | Hs.38489 | SS, SS, ENTH, I_LWEQ, ENTH, I_LWEQ, | _reESTs, Weakly similar to I38022 hypotheti | 8.5 |
| | | | DNA_mis | | |
| 433426 | H69125 | Hs.133525 | , SS, TM | ESTs | 8.5 |
| 424871 | NM_004525 | Hs.153595 | SS, EGF, Idl_recept_a, Idl_recept_b, SS, TM, E | low density lipoprotein-related protein | 8.4 |
| 426215 | AW963419 | Hs.155223 | SS | stanniocalcin 2 | 8.4 |
| 409045 | AA635062 | Hs.50094 | TM | Homo sapiens mRNA; cDNA DKFZp434O0515 (f | 8.4 |
| 435525 | AI831297 | Hs.123310 | TM | ESTs | 8.3 |
| 409203 | AA780473 | Hs.687 | SS, p450, SS, p450 | cytochrome P450, subfamily IVB, polypept | 8.3 |
| 424902 | NM_003866 | Hs.153687 | SS, SS | inositol polyphosphate-4-phosphatase, ty | 8.3 |
| 431725 | X65724 | Hs.2839 | SS, Cys_knot, SS | Norrie disease (pseudoglioma) | 8.3 |
| 418092 | R45154 | Hs.106604 | , death, ZU5, pkinase, Activin_recp, | ESTs | 8.3 |
| 439840 | AW449211 | Hs.105445 | SS | GDNF family receptor alpha 1 | 8.2 |
| 427811 | M81057 | Hs.180884 | SS, Zn_carbOpept, Propep_M14, SS, Propep_{—} | M14carboxypeptidase B1 (tissue) | 8.2 |
| 420807 | AA280627 | Hs.57846 | SS, cpn10 | ESTs | 8.2 |
| 426320 | W47595 | Hs.169300 | SS, TGF-beta, TGFb_propeptide, SS | transforming growth factor, beta 2 | 8.2 |
| 447078 | AW885727 | Hs.301570 | , SS, kazal, | ESTs | 8.1 |
| 415786 | AW419196 | Hs.257924 | SS | hypothetical protein FLJ13782 | 8.1 |
| 410102 | AW248508 | Hs.279727 | SS | Homo sapiens cDNA FLJ14035 fis, clone HE | 8.0 |
| 404347 | | | SS | Target Exon | 8.0 |
| 433687 | AA743991 | | TM | gb: ny57g01.s1 NCI_CGAP_Pr18 Homo sapiens | 8.0 |
| 421373 | AA808229 | Hs.167771 | , SS, IMPDH_C, IMPDH_N, CBS | ESTs | 8.0 |
| 422634 | NM_016010 | Hs.118821 | SS | CGI-62 protein | 7.9 |
| 453310 | X70697 | Hs.553 | TM, SNF, SS, TM, SNF, | solute carrier family 6 (neurotransmitte | 7.9 |
| 435957 | N39015 | Hs.190368 | , SS, TM | ESTs | 7.8 |
| 407771 | AL138272 | Hs.62713 | , TM, cpn60_TCP1, Sema, | ESTs | 7.8 |
| 443646 | AI085198 | Hs.164226 | , TSPN, vwc, tsp_1, EGF, thiored, | ESTs | 7.8 |
| 446142 | AI754693 | Hs.145968 | , TM, cadherin, Cadherin_C_term, | ESTs | 7.7 |
| 444649 | AW207523 | Hs.197628 | , SS, rrm, | ESTs | 7.6 |
| 435147 | AL133731 | Hs.4774 | , TM, SDF, UPAR_LY6, | Homo sapiens mRNA; cDNA DKFZp761C1712 (f | 7.6 |
| 439138 | AI742605 | Hs.193696 | TM | ESTs | 7.6 |
| 429220 | AW207206 | Hs.136319 | SS | ESTs | 7.6 |
| 428804 | AK000713 | Hs.193736 | , SS, UDPGT | hypothetical protein FLJ20706 | 7.5 |
| 453511 | AL031224 | Hs.33102 | SS, SS | transcription factor AP-2 beta (activati | 7.5 |
| 439809 | R41396 | Hs.101774 | SS | hypothetical protein FLJ23045 | 7.5 |
| 414869 | AA157291 | Hs.21479 | SS | ubinuclein 1 | 7.5 |
| 416276 | U41060 | Hs.79136 | SS, TM, TM | LIV-1 protein, estrogen regulated | 7.5 |
| 452862 | AW378065 | Hs.8687 | , SS, Pep_M12B_propep, Reprolysin, tsp_1, | ESTs | 7.4 |
| 452926 | AI742170 | Hs.31297 | , SS, TM | duodenal cytochrome b | 7.4 |
| 453331 | AI240665 | Hs.8895 | , SS, TM, disintegrin, Pep_M12B_propep, Repro | ESTs | 7.3 |
| 420802 | U22376 | Hs.1334 | SS, NA, myb_DNA-binding | v-myb avian myeloblastosis viral oncogen | 7.3 |
| 450603 | R43646 | Hs.12422 | SS | ESTs | 7.2 |
| 422867 | L32137 | Hs.1584 | SS, EGF, tsp_3, SS, E2F_TDP, | cartilage oligomeric matrix protein (COM | 7.2 |
| 418004 | U37519 | Hs.87539 | SS, aldedh, SS, aldedh, | aldehyde dehydrogenase 3 family, member | 7.2 |
| 426451 | AI908165 | Hs.169946 | SS, GATA, | GATA-binding protein 3 (T-cell receptor | 7.1 |
| 450701 | H39960 | Hs.288467 | , SS, LRR | Homo sapiens cDNA FLJ12280 fis, clone MA | 7.1 |
| 419519 | AI198719 | Hs.176376 | SS | ESTs | 7.1 |
| 410555 | U92649 | Hs.64311 | , TM, disintegrin, Reprolysin, | a disintegrin and metalloproteinase doma | 7.1 |
| 433138 | AB029496 | Hs.59729 | SS, ig, Sema, SS, Sema, efhand | semaphorin sem2 | 7.0 |
| 411558 | AA102670 | Hs.70725 | SS, TM, SS, TM | gamma-aminobutyric acid (GABA) A recepto | 7.0 |
| 409079 | W87707 | Hs.82065 | , TM, fn3, | interleukin 6 signal transducer (gp130, | 7.0 |
| 417275 | X63578 | Hs.295449 | SS, efhand, SS, efhand, ras | parvalbumin | 7.0 |
| 432731 | R31178 | Hs.287820 | , SS, fn3, fn1, fn2, fn2, fn1 | fibronectin 1 | 6.9 |
| 442818 | AK001741 | Hs.8739 | WD40, SS | hypothetical protein FLJ10879 | 6.9 |
| 407366 | AF026942 | | , TM, IBR | gb: Homo sapiens cig33 mRNA, partial sequ | 6.8 |
| 427427 | AF077345 | Hs.177936 | SS, lectin_c, SS | ESTs | 6.8 |
| 410785 | AW803341 | | SS | gb: IL2-UM0079-090300-050-D03 UM0079 Homo | 6.7 |
| 401045 | | | ICE_p20, SS, ICE_p10, ICE_p20, ICE_p10, | C11001883*: gi\|6753278\|ref\|NP_033938.1\|c | 6.7 |
| | | | ICE_p | | |
| 418986 | AI123555 | Hs.81796 | , SS, Reprolysin, tsp_1, | ESTs | 6.7 |
| 442082 | R41823 | Hs.7413 | , TM, EPH_Ibd, pkinase, SAM, fn3, | ESTs; calsyntenin-2 | 6.7 |
| 442861 | AA243837 | Hs.57787 | SS | ESTs | 6.6 |
| 418836 | AI655499 | Hs.161712 | , TM, Activin_recp, pkinase, death, ZU5, | ESTs | 6.6 |
| 422060 | R20893 | Hs.325823 | , SS, TM, CD36 | ESTs, Moderately similar to ALU5_HUMAN A | 6.6 |
| 444381 | BE387335 | Hs.283713 | , SS, mito_carr | ESTs, Weakly similar to S64054 hypotheti | 6.6 |
| 404091 | NA | | , TM, 7tm_3, ANF_receptor, | Target Exon | 6.6 |
| 417866 | AW067903 | Hs.82772 | SS, Collagen, COLFI, TSPN, SS, TSPN | collagen, type Xl, alpha 1 | 6.6 |
| 428819 | AL135623 | Hs.193914 | SS, SS | KIAA0575 gene product | 6.5 |
| 410275 | U85658 | Hs.61796 | , SS, Ribosomal_S4e | transcription factor AP-2 gamma (activat | 6.4 |
| 425236 | AW067800 | Hs.155223 | SS | stanniocalcin 2 | 6.2 |
| 415669 | NM_005025 | Hs.78589 | , SS, serpin, | serine (or cysteine) proteinase inhibito | 6.2 |
| 416319 | AI815601 | Hs.79197 | SS, TM, ig, SS, TM | CD83 antigen (activated B lymphocytes, i | 6.2 |
| 412140 | AA219691 | Hs.73625 | , SS, kinesin, | RAB6 interacting, kinesin-like (rabkines | 6.2 |
| 442942 | AW167087 | Hs.131562 | , SS, ig, Sema, pkinase, | ESTs | 6.2 |
| 446163 | AA026880 | Hs.25252 | , SS, TM, fn3, | prolactin receptor | 6.1 |
| 443162 | T49951 | Hs.9029 | filament, SS, filament, filament | DKFZP434G032 protein | 6.1 |
| 409602 | W26713 | Hs.256972 | , SS, TM, DAGKa, DAGKc, | ESTs | 6.1 |
| 428479 | Y00272 | Hs.184572 | , SS, pkinase, pkinase | cell division cycle 2, G1 to S and G2 to | 6.1 |
| 400300 | X03363 | | , SS, TM, pkinase, Recep_L_domain, | receptor tyrosine kinase (c-erb-b2, | 6.1 |
| | | | SH2, PH, FurHER2 | | |
| 433404 | T32982 | Hs.102720 | SS | ESTs | 6.1 |
| 410079 | U94362 | Hs.58589 | Glyco_transf_8, SS | glycogenin 2 | 6.1 |
| 401781 | | | , SS, filament, Pribosyltran, filament, Armad | Target Exon | 6.1 |
| 447359 | NM_012093 | Hs.18268 | SS, adenylatekinase, | adenylate kinase 5 | 6.1 |
| 402230 | NA | | , SS, TM, p450, | Target Exon | 6.1 |
| 427674 | NM_003528 | Hs.2178 | histone, SS, histone, | H2B histone family, member Q | 6.1 |
| 428398 | AI249368 | Hs.98558 | , SS, TM | ESTs | 6.0 |
| 458098 | BE550224 | Hs.74170 | SS | metallothionein 1E (functional) | 6.0 |
| 419968 | X04430 | Hs.93913 | SS, IL6, IL6, | interleukin 6 (interferon, beta 2) | 6.0 |
| 416636 | N32536 | Hs.42645 | , SS, TM | solute carrier family 16 (monocarboxylic | 6.0 |
| 419703 | AI793257 | Hs.128151 | , SS, zf-C2H2, | ESTs | 5.8 |
| 424687 | J05070 | Hs.151738 | SS, Peptidase_M10, fn2, hemopexin, SS, TM, Pep | matrix metalloproteinase 9 (gelatinase B | 5.8 |
| 449679 | AI823951 | Hs.129700 | SS | tolloid-like 1 | 5.8 |
| 421296 | NM_002666 | Hs.103253 | SS | perilipin | 5.8 |
| 442117 | AW664964 | Hs.128899 | , SS, TM | ESTs | 5.7 |
| 400303 | AA242758 | Hs.79136 | , SS, TM | LIV-1 protein, estrogen regulated | 5.7 |
| 419440 | AB020689 | Hs.90419 | SS | KIAA0882 protein | 5.7 |
| 444858 | AI199738 | Hs.208275 | SS | ESTs, Weakly similar to ALUA_HUMAN !!!! | 5.7 |
| 432239 | X81334 | Hs.2936 | SS, Peptidase_M10, hemopexin, SS, Peptidase_{—} | matrix metalloproteinase 13 (collagenase | 5.6 |
| 440705 | AA904244 | Hs.153205 | TM | ESTs | 5.6 |
| 400286 | NA | | SS, TM, ABC_tran, ABC_membrane, SS | C16000922: gi\|7499103\|pir\|\|T20903 hypothe | 5.6 |
| 446466 | H38026 | Hs.308 | arrestin, SS | arrestin 3, retinal (X-arrestin) | 5.5 |
| 423201 | NM_000163 | Hs.125180 | SS, TM, fn3, SS | growth hormone receptor | 5.5 |
| 433043 | W57554 | Hs.125019 | SS | lymphoid nuclear protein (LAF-4) mRNA | 5.5 |
| 439509 | AF086332 | Hs.58314 | , SS, TM, Syntaxin | ESTs | 5.4 |
| 425247 | NM_005940 | Hs.155324 | SS, Peptidase_M10, hemopexin, SS | matrix metalloproteinase 11 (MMP11; stro | 5.4 |
| 409757 | NM_001898 | Hs.123114 | , SS, cystatin, | cystatin SN | 5.4 |
| 425292 | NM_005824 | Hs.155545 | SS | 37 kDa leucine-rich repeat (LRR) protein | 5.4 |
| 448045 | AJ297436 | Hs.20166 | , SS, TM | prostate stem cell antigen | 5.4 |
| 452681 | AF153330 | Hs.30246 | , SS, TM | solute carrier family 19 (thiamine trans | 5.3 |
| 452243 | AL355715 | Hs.28555 | SS | programmed cell death 9 | 5.3 |
| 439310 | AF086120 | Hs.102793 | , SS, TM, UDPGT, casein_kappa | ESTs | 5.2 |
| 441111 | AI806867 | Hs.126594 | , SS, TM, Phosphodiest, | ESTs | 5.2 |
| 452355 | N54926 | Hs.29202 | TM, 7tm_1, TM | G protein-coupled receptor 34 | 5.2 |
| 427711 | M31659 | Hs.180408 | SS | solute carrier family 25 (mitochondrial | 5.2 |
| 418636 | AW749855 | | , SS, TM, HECT | gb: QV4-BT0534-281299-053-c05 BT0534 Homo | 5.2 |
| 429353 | AL117406 | Hs.200102 | , SS, TM, ABC_tran, ABC_membrane, | ATP-binding cassette transporter MRP8 | 5.1 |
| 441690 | R81733 | Hs.33106 | , SS, HECT, zf-UBR1, PABP, 14-3-3, | ESTs | 5.1 |
| 430447 | W17064 | Hs.332848 | SS | SWI/SNF related, matrix associated, acti | 5.1 |
| 429698 | AI685086 | Hs.26339 | , SS, ras, | ESTs, Weakly similar to S21348 probable | 5.1 |
| 425325 | X52730 | Hs.1892 | SS, NNMT_PNMT_TEMT, SS, | phenylethanolamine N-methyltransferase | 5.1 |
| | | | NNMT_PNMT_TEMT, STAR | | |
| 423600 | AI633559 | Hs.310359 | SS | ESTs | 5.1 |
| 414737 | AI160386 | Hs.125087 | SS | ESTs | 5.1 |
| 403593 | NA | | , CIDE-N, pkinase | Target Exon | 5.1 |
| 407758 | D50915 | Hs.38365 | SS, SS | KIAA0125 gene product | 5.0 |
| 445234 | AW137636 | Hs.146059 | , SS, TM | ESTs | 5.0 |
| 411165 | NM_000169 | Hs.69089 | SS, Melibiase, BTK, PH, pkinase, SH2, SH3, Ribo | galactosidase, alpha | 4.9 |
| 420633 | NM_014581 | Hs.274480 | SS, lipocalin, SS, lipocalin | odorant-binding protein 2A | 4.9 |
| 414117 | W88559 | Hs.1787 | , TM, ion_trans, K_tetra, | proteolipid protein 1 (Pelizaeus-Merzbac | 4.9 |
| 416783 | AA206186 | Hs.79889 | SS, TM, TM | monocyte to macrophage differentiation-a | 4.9 |
| 401093 | | | TM, LRRCT, TM, LRRCT, | C12000586*: gi\|6330167\|dbj\|BAA86477.1\| (A | 4.9 |
| 411096 | U80034 | Hs.68583 | Peptidase_M3, | mitochondrial intermediate peptidase | 4.9 |
| 457411 | AW085961 | Hs.130093 | SS | ESTs | 4.9 |
| 436007 | AI247716 | Hs.232168 | , SS, adh_zinc, | ESTs | 4.9 |
| 450506 | NM_004460 | Hs.418 | SS, DPPIV_N_term, Peptidase_S9, SS, | fibroblast activation protein, alpha | 4.9 |
| | | | DPPIV_N_{—} | | |
| 417975 | AA641836 | Hs.30085 | , SS, trypsin | hypothetical protein FLJ23186 | 4.9 |
| 421072 | AI215069 | Hs.89113 | SS | ESTs | 4.8 |
| 427032 | AF012023 | Hs.173274 | , SS, 14-3-3 | integrin cytoplasmic domain-associated p | 4.8 |
| 447752 | M73700 | Hs.105938 | SS, transferrin, 7tm_1, transferrin, | lactotransferrin | 4.8 |
| 403199 | NA | | SS, TM, Folate_carrier, SS, TM, Folate_carrie | NM_025243*: Homo sapiens solute carrier f | 4.8 |
| 427122 | AW057736 | Hs.323910 | , SS, TM, pkinase, Recep_L_domain, | receptor tyrosine kinase (c-erb-b2, | 4.8 |
| | | | SH2, PH, FurHER2 | | |
| 445900 | AF070526 | Hs.13429 | , SS, Ca_channel_B, | Homo sapiens clone 24787 mRNA sequence | 4.7 |
| 413048 | M93221 | Hs.75182 | SS, TM, lectin_c, fn2, Ricin_B_lectin, SS, TM | mannose receptor, C type 1 | 4.7 |
| 419563 | AA526235 | Hs.193162 | SS | Homo sapiens cDNA FLJ11983 fis, clone HE | 4.7 |
| 442432 | BE093589 | Hs.38178 | SS | hypothetical protein FLJ23468 | 4.6 |
| 452093 | AA447453 | Hs.27860 | , SS, TM, 7tm_1, | Homo sapiens mRNA; cDNA DKFZp586M0723 (f | 4.6 |
| 442323 | AW016669 | Hs.29190 | , SS, TM, CBS, voltage_CLC | ESTs | 4.6 |
| 450606 | AI668605 | Hs.60380 | , TM, Glyco_hydro_1 | ESTs, Moderately similar to ALU6_HUMAN A | 4.6 |
| 435542 | AA687376 | Hs.269533 | , SS, pkinase, RhoGEF, ig, PH, SH3, | ESTs | 4.6 |
| 417576 | AA339449 | Hs.82285 | AIRS, formyl_transf, GARS, SS, GARS, AIRS, for | phosphoribosylglycinamide formyltransfer | 4.6 |
| 446089 | AI860021 | Hs.270651 | , pkinase | ESTs, Moderately similar to A47582 B-cel | 4.6 |
| 445413 | AA151342 | Hs.12677 | SS, UPF0099, SS, UPF0099, | CGI-147 protein | 4.6 |
| 424420 | BE614743 | Hs.146688 | , SS, TM, MAPEG, | prostaglandin E synthase | 4.5 |
| 432378 | AI493046 | Hs.146133 | , SS, TM, UDPGT | ESTs | 4.5 |
| 452190 | H26735 | Hs.91668 | , SS, TM, PH, SH2, Furin-like, pkinase, Recep_L | Homo sapiens clone PP1498 unknown mRNA | 4.5 |
| 434674 | AA831879 | Hs.136985 | , SS, Hist_deacetyl, | ESTs | 4.5 |
| 419986 | AI345455 | Hs.78915 | pkinase, OPR, | GA-binding protein transcription factor, | 4.5 |
| 421582 | AI910275 | Hs.1406 | SS, trefoil, SS, TM, Idl_recept_a, SRCR, tryps | trefoil factor 1 (pS2) | 4.5 |
| 410361 | BE391804 | Hs.62661 | SS, TM, GBP, TM, GBP | guanylate binding protein 1, interferon- | 4.5 |
| 426327 | W03242 | Hs.44898 | SS | Homo sapiens clone TCCCTA00151 mRNA sequ | 4.5 |
| 406639 | M97711 | | SS, SS, ig, | gb: Human T-cell receptor (V beta 18.1, J | 4.5 |
| 452834 | AI638627 | Hs.105685 | , SS, DEAD, Fork_head | KIAA1688 protein | 4.5 |
| 427315 | AA179949 | Hs.175563 | SS | Homo sapiens mRNA; cDNA DKFZp564N0763 (f | 4.4 |
| 446733 | AA863360 | Hs.26040 | , SS, TM, p450, | ESTs, Weakly similar to fatty acid omega | 4.4 |
| 442118 | AA976718 | Hs.202242 | , ig, Sema, | ESTs | 4.4 |
| 421524 | AA312082 | Hs.105445 | SS | GDNF family receptor alpha 1 | 4.4 |
| 453060 | AW294092 | Hs.21594 | , SS, ras, Y_phosphatase, ras | hypothetical protein MGC15754 | 4.4 |
| 453403 | BE466639 | Hs.61779 | , SS, HMG_box, filament, | Homo sapiens cDNA FLJ13691 fis, clone PL | 4.4 |
| 444301 | AK000136 | Hs.10760 | SS, LRR, SS | asporin (LRR class 1) | 4.4 |
| 453619 | H87648 | Hs.33922 | SS | Homo sapiens, clone MGC: 9084, mRNA, comp | 4.3 |
| 432656 | NM_000246 | Hs.3076 | SS, LRR, | MHC class II transactivator | 4.3 |
| 426384 | AI472078 | Hs.303662 | , SS, ArfGap, | ESTs | 4.3 |
| 431701 | AW935490 | Hs.14658 | , SS, BIR | Human chromosome 5q13.1 clone 5GB mRNA | 4.3 |
| 416931 | D45371 | Hs.80485 | SS, C1q, Collagen, SS, C1q, | adipose most abundant gene transcript 1 | 4.3 |
| 420854 | AW296927 | | , SS, TM, Peptidase_M1, | gb: UI-H-BW0-ajc-c-07-0-UI.s1 NCI_CGAP_Su | 4.3 |
| 418867 | D31771 | Hs.89404 | SS, homeobox, homeobox, | msh (Drosophila) homeo box homolog 2 | 4.3 |
| 443514 | BE464288 | Hs.141937 | , SS, TM, MIP, | ESTs | 4.3 |
| 447499 | AW262580 | Hs.147674 | , SS, TM, cadherin, cadherin | protocadherin beta 16 | 4.3 |
| 441560 | F13386 | Hs.7888 | , pkinase, | Homo sapiens clone 23736 mRNA sequence | 4.3 |
| 409064 | AA062954 | Hs.141883 | , SS, CUB, | ESTs | 4.3 |
| 422667 | H25642 | Hs.133471 | , SS, TM, FMO-like | ESTs | 4.3 |
| 454032 | W31790 | Hs.194293 | , SS, TM | ESTs, Weakly similar to I54374 gene NF2 | 4.3 |
| 432663 | AI984317 | Hs.122589 | TM | ESTs | 4.3 |
| 401747 | | | , SS, filament, filament | Homo sapiens keratin 17 (KRT17) | 4.3 |
| 432882 | NM_013257 | Hs.279696 | pkinase, pkinase_C, | serum/glucocorticoid regulated kinase-li | 4.2 |
| 437036 | AI571514 | Hs.133022 | , SS, TM | ESTs | 4.2 |
| 447754 | AW073310 | Hs.163533 | , pkinase, | Homo sapiens cDNA FLJ14142 fis, clone MA | 4.2 |
| 443194 | AI954968 | Hs.279009 | , SS, TM | matrix Gla protein | 4.2 |
| 451871 | AI821005 | Hs.118599 | , SS, GDNF, | ESTs | 4.2 |
| 457211 | AW972565 | Hs.32399 | WH1, WH1 | ESTs, Weakly similar to S51797 vasodilat | 4.2 |
| 421566 | NM_000399 | Hs.1395 | zf-C2H2, SS | early growth response 2 (Krox-20 (Drosop | 4.2 |
| 431657 | AI345227 | Hs.105448 | , SS, TM, pkinase | ESTs, Weakly similar to B34087 hypotheti | 4.1 |
| 427899 | AA829286 | Hs.332053 | , SS, SAA_proteins, ABC_membrane, ABC_tran, | serum amyloid A1 | 4.1 |
| 444779 | AI192105 | Hs.147170 | SS | ESTs | 4.1 |
| 442295 | AI827248 | Hs.224398 | , COLFI, vwc, Collagen, | Homo sapiens cDNA FLJ11469 fis, clone HE | 4.1 |
| 436396 | AI683487 | Hs.152213 | , wnt, | wingless-type MMTV integration site fami | 4.1 |
| 446039 | AI150491 | Hs.90756 | , TM, Glyco_hydro_1 | ESTs | 4.1 |
| 422938 | NM_001809 | Hs.1594 | , SS, TM, thiolase, | centromere protein A (17 kD) | 4.1 |
| 406922 | S70284 | | SS, TM, Desaturase, SS | gb: stearoyl-CoA desaturase [human, adipo | 4.1 |
| 439285 | AL133916 | Hs.172572 | , SS, ig, pkinase, LRRNT, LRRCT, | hypothetical protein FLJ20093 | 4.1 |
| 424800 | AL035588 | Hs.153203 | HLH, SS | MyoD family inhibitor | 4.1 |
| 429922 | Z97630 | Hs.226117 | , SS, TM, linker_histone, 7tm_1 | H1 histone family, member 0 | 4.1 |
| 447178 | AW594641 | Hs.192417 | , SS, TM | ESTs | 4.0 |
| 409038 | T97490 | Hs.50002 | SS, IL8, SS, IL8 | small inducible cytokine subfamily A (Cy | 4.0 |
| 452747 | BE153855 | Hs.61460 | , SS, HLH | lg superfamily receptor LNIR | 4.0 |
| 420139 | NM_005357 | Hs.95351 | , SS, TM, p450, | lipase, hormone-sensitive | 4.0 |
| 408877 | AA479033 | Hs.130315 | , SS, TM | ESTs, Weakly similar to A47582 B-cell gr | 4.0 |
| 403329 | NA | | SS, SS | Target Exon | 4.0 |
| 439926 | AW014875 | Hs.137007 | SS | ESTs | 4.0 |
| 430832 | AI073913 | Hs.100686 | SS | ESTs, Weakly similar to JE0350 Anterior | 4.0 |
| 432481 | AW451645 | Hs.151504 | , SS, Collagen, COLFI, TSPN, | Homo sapiens cDNA FLJ11973 fis, clone HE | 4.0 |
| 452410 | AL133619 | Hs.29383 | , SS, TM, ras | Homo sapiens mRNA; cDNA DKFZp434E2321 (f | 4.0 |
| 418661 | NM_001949 | Hs.1189 | SS | E2F transcription factor 3 | 4.0 |
| 431958 | X63629 | Hs.2877 | SS, TM, Cadherin_C_term, cadherin, SS, TM, | cadcadherin 3, type 1, P-cadherin (placenta | 4.0 |
| 425071 | NM_013989 | Hs.154424 | SS, T4_deiodinase, T4_deiodinase, | deiodinase, iodothyronine, type II | 4.0 |
| 447197 | R36075 | | , TM, SDF, | gb: yh88b01.s1 Soares placenta Nb2HP Homo | 4.0 |
| 428722 | U76456 | Hs.190787 | , SS, TIMP, | tissue inhibitor of metalloproteinase 4 | 3.9 |
| 428330 | L22524 | Hs.2256 | SS, Peptidase_M10, SS, Peptidase_M10, | metalloproteinase 7 (MMP7; uterin | 3.9 |
| | | | hemopematrix | | |
| 423242 | AL039402 | Hs.125783 | SS | DEME-6 protein | 3.9 |
| 449048 | Z45051 | Hs.22920 | SS, SS, TM | similar to S68401 (cattle) glucose induc | 3.9 |
| 414831 | M31158 | Hs.77439 | , SS, cNMP_binding, Rlla, HMG_box | protein kinase, cAMP-dependent, regulato | 3.9 |
| 413589 | AW452631 | Hs.313803 | , SS, abhydrolase | ESTs, Highly similar to AF157833 1 noncl | 3.8 |
| 408875 | NM_015434 | Hs.48604 | SS | DKFZP434B168 protein | 3.8 |
| 418629 | BE247550 | Hs.86859 | SS, SH2, PH, SS, TM, PH, SH2, Furin-like, pkinas | growth factor receptor-bound protein 7 | 3.8 |
| 450787 | AB006190 | Hs.25475 | SS, TM, MIP, SS, TM, MIP, | aquaporin 7 | 3.8 |
| 414870 | N72264 | Hs.300670 | SS | KIAA1204 protein | 3.8 |
| 450325 | AI935962 | Hs.26289 | SS | ESTs | 3.8 |
| 407633 | NM_007069 | Hs.37189 | TM, TM | similar to rat HREV107 | 3.8 |
| 426172 | AA371307 | Hs.125056 | , SS, DENN | ESTs | 3.8 |
| 442262 | BE170651 | Hs.8700 | , SS, START, | deleted in liver cancer 1 | 3.8 |
| 427961 | AW293165 | Hs.143134 | SS | ESTs | 3.8 |
| 445563 | AW873606 | Hs.149006 | , SS, WH1, WH1 | ESTs | 3.8 |
| 403943 | | | p450, SS, p450 | C5000355: gi\|4503225\|ref\|NP_000765.1\|cyt | 3.8 |
| 408761 | AA057264 | Hs.238936 | , SS, TM, 7tm_1, | ESTs, Weakly similar to (defline not ava | 3.8 |
| 423279 | AW959861 | Hs.290943 | SS | ESTs | 3.8 |
| 420440 | NM_002407 | Hs.97644 | , SS, SRCR, Uteroglobin | mammaglobin 2 (MGB2; mammaglobin B; lip | 3.8 |
| 445107 | AI208121 | Hs.147313 | , SS, TM | ESTs, Weakly similar to I38022 hypotheti | 3.7 |
| 428303 | AW974476 | Hs.183601 | SS, RGS, RGS, RGS | regulator of G-protein signalling 16 | 3.7 |
| 411667 | BE160198 | | TM | gb: QV1-HT0413-010200-059-h03 HT0413 Homo | 3.7 |
| 427809 | M26380 | Hs.180878 | , SS, lipase, PLAT, Sec7, PH, | lipoprotein lipase | 3.7 |
| 418203 | X54942 | Hs.83758 | CKS, SS, CKS, | CDC28 protein kinase 2 | 3.7 |
| 430376 | AW292053 | Hs.12532 | SS | chromosome 1 open reading frame 21 | 3.7 |
| 444190 | AI878918 | Hs.10526 | SS | cysteine and glycine-rich protein 2 | 3.7 |
| 433495 | AW373784 | Hs.71 | SS, ig, MHC_I, connexin, SCAN, SS, TM | alpha-2-glycoprotein 1, zinc | 3.7 |
| 429638 | AI916662 | Hs.211577 | SS, TM, SS | kinectin 1 (kinesin receptor) | 3.7 |
| 454071 | AI041793 | Hs.42502 | , TM, 7tm_1, | ESTs | 3.7 |
| 451859 | H44491 | Hs.252938 | , SS, TM, EGF, Idl_recept_a, Idl_recept_b, EGF | ESTs, Weakly similar to ALU1_HUMAN ALU S | 3.7 |
| 420281 | AI623693 | Hs.191533 | , SS, AAA, | ESTs | 3.7 |
| 427691 | AW194426 | Hs.20726 | , SS, Glycos_transf_2, | ESTs | 3.7 |
| 428824 | W23624 | Hs.173059 | SS | ESTs | 3.7 |
| 424676 | Y08565 | Hs.151678 | Glycos_transf_2, Ricin_B_lectin, SS, Glycos | UDP-N-acetyl-alpha-D-galactosamine: polyp | 3.7 |
| 418026 | BE379727 | Hs.83213 | lipocalin, SS, lipocalin, lipocalin, ferriti | fatty acid binding protein 4, adipocyte | 3.7 |
| 457465 | AW301344 | Hs.122908 | , SS, Pribosyltran, Sulfatase | DNA replication factor | 3.7 |
| 417601 | NM_014735 | Hs.82292 | PHD, pkinase, SS | KIAA0215 gene product | 3.7 |
| 407999 | AI126271 | Hs.49433 | SS | ESTs, Weakly similar to YZ28_HUMAN HYPOT | 3.7 |
| 425548 | AA890023 | Hs.1906 | SS, TM, fn3, SS, TM, fn3, | prolactin receptor | 3.7 |
| 446619 | AU076643 | Hs.313 | , SS, TM, efhand, ion_trans | secreted phosphoprotein 1 (osteopontin, | 3.7 |
| 411213 | AA676939 | Hs.69285 | SS, TM, CUB, F5_F8_type_C, MAM, SS, | neuropilin 1 | 3.6 |
| | | | TM, CUB, F5_{—} | | |
| 406625 | Y13647 | Hs.119597 | SS, TM, Desaturase, SS | stearoyl-CoA desaturase (delta-9-desatur | 3.6 |
| 417511 | AL049176 | Hs.82223 | SS | chordin-like | 3.6 |
| 428769 | AW207175 | Hs.106771 | , SS, 7tm_1, SPRY, | ESTs | 3.6 |
| 407137 | T97307 | | , SS, TM, GDA1_CD39 | gb: ye53h05.s1 Soares fetal liver spleen | 3.6 |
| 401866 | | | , SS, filament, | Target Exon | 3.6 |
| 451195 | U10492 | Hs.438 | SS, homeobox, Ets, SS, homeobox, | mesenchyme homeo box 1 | 3.6 |
| 426044 | AA502490 | Hs.336695 | SS | ESTs | 3.6 |
| 426310 | NM_000909 | Hs.169266 | SS, TM, 7tm_1, | neuropeptide Y receptor Y1 | 3.6 |
| 440029 | AW089705 | Hs.293711 | SS | ESTs, Weakly similar to S64329 probable | 3.6 |
| 408573 | AA284775 | Hs.43148 | , SS, TM, PMP22_Claudin, | ESTs | 3.6 |
| 431830 | Y16645 | Hs.271387 | , SS, TM, IL8 | small inducible cytokine subfamily A (Cy | 3.6 |
| 444781 | NM_014400 | Hs.11950 | , SS, PH, lactamase_B | GPI-anchored metastasis-associated prote | 3.6 |
| 431493 | AI791493 | Hs.129873 | , SS, p450, p450 | ESTs, Weakly similar to A36036 cytochrom | 3.6 |
| 414175 | AI308876 | Hs.103849 | , TM, hemopexin, Peptidase_M10, hemopexin, Pe | hypothetical protein DKFZp761D112 | 3.6 |
| 411789 | AF245505 | Hs.72157 | ig, LRRCT, | DKFZP564I1922 protein | 3.6 |
| 418851 | AI417828 | Hs.192435 | , SS, TM | ESTs | 3.5 |
| 453968 | AA847843 | Hs.62711 | , SS, HMG_box, | Homo sapiens, clone IMAGE: 3351295, mRNA | 3.5 |
| 407104 | S57296 | Hs.323910 | , SS, TM, SH2, PH, pkinase, | avian erythroblastic leukemia v | 3.5 |
| | | | Recep_L_domain,Furv-erb-b2 | | |
| 449051 | AW961400 | Hs.333526 | SS | HER2 receptor tyrosine kinase (c-erb-b2, | 3.5 |
| 434398 | AA121098 | Hs.3838 | pkinase, POLO_box, SS, pkinase, POLO_box, | serum-inducible kinase | 3.5 |
| 454042 | H22570 | Hs.172572 | , SS, ig, pkinase, LRRNT, LRRCT, | hypothetical protein FLJ20093 | 3.5 |
| 459496 | AA808940 | Hs.274450 | , SS, TM, KRAB, SCAN, zf-C2H2, ig | EST | 3.5 |
| 414998 | NM_002543 | Hs.77729 | , SS, TM | oxidised low density lipoprotein (lectin | 3.5 |
| 407756 | AA116021 | Hs.38260 | SS, UCH-1, UCH-2, SS, TM, G_glu_transpept | ubiquitin specific protease 18 | 3.5 |
| 442101 | AI651930 | Hs.135684 | SS | ESTs | 3.5 |
| 449722 | BE280074 | Hs.23960 | cyclin, SS, TM, cyclin, | cyclin B1 | 3.5 |
| 452554 | AW452434 | Hs.58006 | SS | ESTs, Weakly similar to ALU5_HUMAN ALU S | 3.5 |
| 421991 | NM_014918 | Hs.110488 | SS | KIAA0990 protein | 3.4 |
| 420058 | AK001423 | Hs.94694 | SS | Homo sapiens cDNA FLJ10561 fis, clone NT | 3.4 |
| 425776 | U25128 | Hs.159499 | SS, TM, 7tm_2, SS, TM, 7tm_2 | parathyroid hormone receptor 2 | 3.4 |
| 407846 | AA426202 | Hs.40403 | , TM, ABC_membrane, ABC_tran, Ribosomal_S4 | eCbp/p300-interacting transactivator, wit | 3.4 |
| 406925 | L34041 | Hs.9739 | , SS, TM, transport_prot, SWIB, RhoGAP, | glycerol-3-phosphate dehydrogenase 1 (so | 3.4 |
| | | | DAG_PE | | |
| 445873 | AA250970 | Hs.251946 | , SS, rrm, PABP, pkinase, 14-3-3, rrm | poly(A)-binding protein, cytoplasmic 1-l | 3.4 |
| 418054 | NM_002318 | Hs.83354 | , SS, TM, mito_carr, Lysyl_oxidase | lysyl oxidase-like 2 | 3.4 |
| 414921 | BE390551 | Hs.77628 | SS, START, SS, START, NNMT_PNMT_TEMT, | steroidogenic acute regulatory protein r | 3.4 |
| 452268 | NM_003512 | Hs.26777 | SS, histone, Calc_CGRP_IAPP, ig, MHC_I, | H2A histone family, member L | 3.4 |
| | | | SPRY, | | |
| 428862 | NM_000346 | Hs.2316 | SS, HMG_box, | SRY (sex determining region Y)-box 9 (ca | 3.4 |
| 412520 | AA442324 | Hs.795 | histone, SS, histone, BolA | H2A histone family, member O | 3.4 |
| 410530 | M25809 | Hs.64173 | ATP-synt_ab, SS, 7tm_1, ATP-synt_ab | ATPase, H transporting, lysosomal (vacuo | 3.4 |
| 401780 | | | filament, SS, filament, filament | NM_005557*: Homo sapiens keratin 16 (foca | 3.4 |
| 447131 | NM_004585 | Hs.17466 | TM | retinoic acid receptor responder (tazaro | 3.4 |
| 418334 | AA319233 | Hs.5521 | , SS, TM, Ribosomal_L27e, | ESTs | 3.4 |
| 415138 | C18356 | Hs.295944 | , Kunitz_BPTI, | tissue factor pathway inhibitor 2 | 3.4 |
| 421168 | AF182277 | Hs.330780 | SS, p450, SS | cytochrome P450, subfamily IIB (phenobar | 3.4 |
| 431473 | AA825686 | Hs.321176 | SS | ESTs, Weakly similar to S65824 reverse t | 3.4 |
| 421379 | Y15221 | Hs.103982 | SS, IL8, | small inducible cytokine subfamily B (Cy | 3.4 |
| 411984 | NM_005419 | Hs.72988 | SH2, STAT, SS, STAT | signal transducer and activator of trans | 3.4 |
| 408101 | AW968504 | Hs.123073 | , pkinase, | CDC2-related protein kinase 7 | 3.4 |
| 405366 | | | RhoGEF, PH, SS, RhoGEF, PH, | NM_003371*: Homo sapiens vav 2 oncogene ( | 3.4 |
| 414612 | BE274552 | Hs.76578 | SAP, SS, FG-GAP, vwa | protein inhibitor of activated STAT3 | 3.4 |
| 411393 | AW797437 | Hs.69771 | SS, sushi, trypsin, vwa, rrm, fibrinogen_C, fn | B-factor, properdin | 3.3 |
| 435767 | H73505 | Hs.117874 | , SS, Peptidase_S8, P, Peptidase_S8, P | ESTs | 3.3 |
| 416406 | D86961 | Hs.79299 | , SS, TM | lipoma HMGIC fusion partner-like 2 | 3.3 |
| 433068 | NM_006456 | Hs.288215 | , SS, Pribosyltran, | sialyltransferase | 3.3 |
| 445462 | AA378776 | Hs.288649 | SS, SS | hypothetical protein MGC3077 | 3.3 |
| 439452 | AA918317 | Hs.57987 | SS, SS | B-cell CLL/lymphoma 11B (zinc finger pro | 3.3 |
| 452017 | AF109302 | Hs.27495 | SS | prostate cancer associated protein 7 | 3.3 |
| 409099 | AK000725 | Hs.50579 | SS | hypothetical protein FLJ20718 | 3.3 |
| 452106 | AI141031 | Hs.21342 | SS | ESTs | 3.3 |
| 447519 | U46258 | Hs.339665 | SS | ESTs | 3.3 |
| 426928 | AF037062 | Hs.172914 | , SS, adh_short, TGF-beta, TGFb_propeptide | retinol dehydrogenase 5 (11-cis and 9-ci | 3.3 |
| 438825 | BE327427 | Hs.79953 | , SS, TM, histone, ANF_receptor, guanylate_cy | ESTs | 3.3 |
| 414575 | H11257 | Hs.22968 | , SS, pkinase, ig, | Homo sapiens clone IMAGE: 451939, mRNA se | 3.3 |
| 417837 | AL079905 | Hs.1103 | SS, TGFb_propeptide, TGF-beta, SS | transforming growth factor, beta 1 | 3.3 |
| 422128 | AW881145 | | SS | gb: QV0-OT0033-010400-182-a07 OT0033 Homo | 3.3 |
| 445941 | AI267371 | Hs.172636 | SS, SS, lipoxygenase, PLAT | ESTs | 3.3 |
| 429973 | AI423317 | Hs.164680 | , SS, T-box, UDPGT | ESTs | 3.3 |
| 444542 | AI161293 | Hs.280380 | SS, SS, Peptidase_M1, EGF, ig, lectin_c, sushi | aminopeptidase | 3.3 |
| 459561 | AI547306 | Hs.134981 | SS | ESTs | 3.3 |
| 425741 | AF052152 | Hs.159412 | , pkinase, | Homo sapiens clone 24628 mRNA sequence | 3.3 |
| 426501 | AW043782 | Hs.293616 | SS | ESTs | 3.3 |
| 456508 | AA502764 | Hs.123469 | SS | ESTs, Weakly similar to AF208855 1 BM-01 | 3.3 |
| 434228 | Z42047 | Hs.283978 | , SS, TM, 7tm_1 | Homo sapiens PRO2751 mRNA, complete cds | 3.3 |
| 415752 | BE314524 | Hs.78776 | TM | putative transmembrane protein | 3.3 |
| 400419 | AF084545 | | , SS, Peptidase_M1, | Target | 3.3 |
| 439750 | AL359053 | Hs.57664 | , TM, integrin_B, Ricin_B_lectin, rrm | Homo sapiens mRNA full length insert cDN | 3.3 |
| 423858 | AL137326 | Hs.133483 | , SS, TM | Homo sapiens mRNA; cDNA DKFZp434B0650 (f | 3.3 |
| 428514 | AW236861 | Hs.193139 | , SS, START, NNMT_PNMT_TEMT, | ESTs | 3.3 |
| 428698 | AA852773 | Hs.334838 | SS | KIAA1866 protein | 3.3 |
| 448988 | Y09763 | Hs.22785 | SS, TM, TM | gamma-aminobutyric acid (GABA) A recepto | 3.3 |
| 432072 | N62937 | Hs.269109 | , Sema, ig, | ESTs | 3.3 |
| 417433 | BE270266 | Hs.82128 | SS, TM, LRRCT, LRRNT, LRR, TM, LRRCT, | 5T4 oncofetal trophoblast glycoprotein | 3.3 |
| 452194 | AI694413 | Hs.332649 | , SS, TM, 7tm_3, ANF_receptor, sushi | olfactory receptor, family 2, subfamily | 3.2 |
| 444051 | N48373 | Hs.10247 | , SS, ig, | activated leucocyte cell adhesion molecu | 3.2 |
| 420042 | AW015140 | Hs.161723 | , SS, CUB, | ESTs | 3.2 |
| 457292 | AI921270 | Hs.334882 | SS, TM, SS, TM, G-patch | hypothetical protein FLJ14251 | 3.2 |
| 421458 | NM_003654 | Hs.104576 | SS | carbohydrate (keratan sulfate Gal-6) sul | 3.2 |
| 431104 | AW970859 | Hs.313503 | , Sema, ig, | ESTs | 3.2 |
| 443767 | BE562136 | Hs.9736 | , SS, PCI, RasGEF, hormone_rec, zf-C4, | proteasome (prosome, macropain) 26S subu | 3.2 |
| 419589 | AW973708 | Hs.201925 | , FGF, | Homo sapiens cDNA FLJ13446 fis, clone PL | 3.2 |
| 415447 | Z97171 | Hs.78454 | SS, OLF, OLF, OLF, Ribosomal_L4 | myocilin, trabecular meshwork inducible | 3.2 |
| 443464 | BE548446 | Hs.5167 | SS, TM, SSF, SS, TM | Homo sapiens mRNA; cDNA DKFZp434F152 (fr | 3.2 |
| 423431 | AA326062 | | , SS, p450, p450 | gb: EST29171 Cerebellum II Homo sapiens c | 3.2 |
| 413278 | BE563085 | Hs.833 | , SS, TM, ubiquitin, laminin_G, laminin_EGF, k | interferon-stimulated protein, 15 kDa | 3.2 |
| 458451 | AW297181 | Hs.195922 | , SS, Ribosomal_L14 | ESTs | 3.2 |
| 440449 | AA885430 | Hs.201925 | , FGF, | Homo sapiens cDNA FLJ13446 fis, clone PL | 3.2 |
| 413753 | U17760 | Hs.75517 | SS, laminin_EGF, laminin_Nterm, adh_short, S | laminin, beta 3 (nicein (125 kD), kalinin | 3.2 |
| 434876 | AF160477 | Hs.61460 | , SS, HLH | lg superfamily receptor LNIR | 3.2 |
| 435575 | AF213457 | Hs.44234 | SS, ig, SS, TM | triggering receptor expressed on myeloid | 3.2 |
| 415773 | R21651 | Hs.324725 | , SS, TM, Ribosomal_S3Ae, G-gamma | ESTs, Moderately similar to A47582 B-cel | 3.2 |
| 446440 | AV658411 | Hs.42656 | SS | KIAA1681 protein | 3.2 |
| 450847 | NM_003155 | Hs.25590 | , SS, homeobox, | stanniocalcin 1 | 3.2 |
| 426075 | AW513691 | Hs.270149 | , SS, fn3, | ESTs, Weakly similar to 2109260A B cell | 3.2 |
| 452110 | T47667 | Hs.28005 | , SS, TM, Activin_recp, pkinase | Homo sapiens cDNA FLJ11309 fis, clone PL | 3.2 |
| 439963 | AW247529 | Hs.6793 | , TM, p450, Ets | platelet-activating factor acetylhydrola | 3.2 |
| 402837 | NA | | SS | ENSP00000241312*: DJ947L8.1.8 (novel Sush | 3.2 |
| 439451 | AF086270 | Hs.278554 | , SS, Chromo_shadow, chromo, | heterochromatin-like protein 1 | 3.1 |
| 406654 | L34041 | Hs.9739 | , SS, TM, transport_prot, SWIB, RhoGAP, | glycerol-3-phosphate dehydrogenase 1 (so | 3.1 |
| | | | DAG_PE | | |
| 417315 | AI080042 | Hs.336901 | , SS, RNA_pol_A, RNA_pol_A2, | ribosomal protein S24 | 3.1 |
| | | | Ribosomal_S24e, | | |
| 413011 | AW068115 | Hs.821 | SS, LRR, LRRNT, SS, LRRNT, LRR, | biglycan | 3.1 |
| 414987 | AA524394 | Hs.294022 | , SS, connexin, hormone_rec, zf-C4, connexin | hypothetical protein FLJ14950 | 3.1 |
| 429197 | H24471 | Hs.26930 | , SS, Gelsolin, | ESTs, Weakly similar to T20272 hypotheti | 3.1 |
| 448030 | N30714 | Hs.325960 | , SS, TM | membrane-spanning 4-domains, subfamily A | 3.1 |
| 407604 | AW191962 | Hs.249239 | , SS, TM, C1q, | collagen, type VIII, alpha 2 | 3.1 |
| 419092 | J05581 | Hs.89603 | SS, TM, SEA, | mucin 1, transmembrane | 3.1 |
| 456672 | AK002016 | Hs.114727 | , SS, PK, PK | Homo sapiens, clone MGC: 16327, mRNA, com | 3.1 |
| 443171 | BE281128 | Hs.9030 | SS, TM, 7tm_1, rrm, SS | TONDU | 3.1 |
| 452256 | AK000933 | Hs.28661 | , TM, GDI, 7tm_1, | Homo sapiens cDNA FLJ10071 fis, clone HE | 3.1 |
| 432201 | AI538613 | Hs.298241 | SS, TM, trypsin, SS, TM, trefoil, trypsin, tref | Transmembrane protease, serine 3 | 3.1 |
| 406642 | AJ245210 | | SS | gb: Homo sapiens mRNA for immunoglobulin | 3.1 |
| 400903 | NA | | SS | Target Exon | 3.1 |
| 434408 | AI031771 | Hs.132586 | , SS, Glyco_hydro_2 | ESTs | 3.1 |
| 452994 | AW962597 | Hs.31305 | SS, WD40, SS, WD40, | KIAA1547 protein | 3.1 |
| 445903 | AI347487 | Hs.132781 | fn3, SS, TM, EF1BD | class I cytokine receptor | 3.1 |
| 424364 | AW383226 | Hs.201189 | SS | ESTs, Weakly similar to G01763 atrophin- | 3.1 |
| 410196 | AI936442 | Hs.59838 | UBACT_repeat, SS, UBACT_repeat, ThiF_family | hypothetical protein FLJ10808 | 3.1 |
| 419150 | T29618 | Hs.89640 | , TM, pkinase, fn3, | TEK tyrosine kinase, endothelial (venous | 3.1 |
| 433417 | AA587773 | Hs.8859 | , SS, SRCR, | Homo sapiens, Similar to RIKEN cDNA 5830 | 3.1 |
| 418624 | AI734080 | Hs.104211 | , Sema, ig, | ESTs | 3.1 |
| 436291 | BE568452 | Hs.5101 | , SS, abhydrolase, | protein regulator of cytokinesis 1 | 3.1 |
| 411000 | N40449 | Hs.201619 | SS | ESTs, Weakly similar to S38383 SEB4B pro | 3.1 |
| 412519 | AA196241 | Hs.73980 | SS, Troponin, Hemagglutinin, SS, TM, C2, Tropo | troponin T1, skeletal, slow | 3.1 |
| 450223 | AA418204 | Hs.241493 | , SS, pro_isomerase, | natural killer-tumor recognition sequenc | 3.1 |
| 422790 | AA809875 | Hs.25933 | , TM, histone, Sec1, histone, sugar_tr | ESTs | 3.1 |
| 424269 | AW137691 | Hs.199754 | , SS, TM, 7tm_2, GPS | ESTs | 3.1 |
| 435854 | AJ278120 | Hs.4996 | , SS, WD40 | putative ankyrin-repeat containing prote | 3.1 |
| 447388 | AW630534 | Hs.76277 | , SS, TM, rrm, oxidored_q6, oxidored_q6 | Homo sapiens, clone MGC: 9381, mRNA, comp | 3.1 |
| 451631 | R00866 | | SS | gb: ye79c02.s1 Soares fetal liver spleen | 3.0 |
| 448105 | AW591433 | Hs.298241 | , SS, TM, trefoil, trypsin, trefoil | Transmembrane protease, serine 3 | 3.0 |
| 438637 | BE500941 | Hs.126730 | , TM, PH, | ESTs, Weakly similar to KIAA1214 protein | 3.0 |
| 423024 | AA593731 | Hs.325823 | , SS, TM, CD36 | ESTs, Moderately similar to ALU5_HUMAN A | 3.0 |
| 456592 | R91600 | | , SS, Ran_BP1, LIM, Ran_BP1, GRIP, TPR, LIM | gb: yq10c02.r1 Soares fetal liver spleen | 3.0 |
| 425920 | AL049977 | Hs.162209 | SS, TM, SS, TM, PMP22_Claudin, | claudin 8 | 3.0 |
| | | | PMP22_Claudin | | |
| 444670 | H58373 | Hs.332938 | , SS, TM | hypothetical protein MGC5370 | 3.0 |
| 401197 | | | arf, arf, | ENSP00000229263*: HSPC213. | 3.0 |
| 437755 | AW204256 | Hs.291887 | , wnt, | ESTs | 3.0 |
| 452560 | BE077084 | Hs.336432 | , SS, rrm, zf-RanBP, pkinase, C2, pkinase_C, DA | ESTs | 3.0 |
| 410274 | AA381807 | Hs.61762 | SS, SS | hypoxia-inducible protein 2 | 3.0 |
| 450098 | W27249 | Hs.8109 | SS | hypothetical protein FLJ21080 | 3.0 |
| 404826 | | | , SS, TM | Target Exon | 3.0 |
| 458389 | H70284 | Hs.160152 | , SS, RA | ESTs, Weakly similar to FPHU alpha-fetop | 3.0 |
| 408196 | AL034548 | Hs.43627 | HMG_box, pkinase, zf-CCHC, SS, TM, | SRY (sex determining region Y)-box 22 | 3.0 |
| | | | HMG_box, | | |
| 433675 | AW977653 | Hs.75319 | , SS, ribonuc_red_sm, | ribonucleotide reductase M2 polypeptide | 3.0 |
| 418848 | AI820961 | Hs.193465 | , death, ZU5, pkinase, Activin_recp, | ESTs | 3.0 |
| 422095 | AI868872 | Hs.282804 | SS, Cu-oxidase, SS, Cu-oxidase, Cu-oxidase | hypothetical protein FLJ22704 | 3.0 |
| 415992 | C05837 | Hs.145807 | , SS, TM | hypothetical protein FLJ13593 | 3.0 |
| 424631 | AA688021 | Hs.179808 | SS | ESTs | 3.0 |
| 409956 | AW103364 | Hs.727 | SS, TGF-beta, TGFb_propeptide, SS, TGF-beta, | inhibin, beta A (activin A, activin AB a | 3.0 |
| 419667 | AU077005 | Hs.92208 | SS, disintegrin, Reprolysin, Pep_M12B_prope | a disintegrin and metalloproteinase doma | 3.0 |
| 450946 | AA374569 | Hs.127698 | SS | ESTs, Moderately similar to 2109260A B c | 3.0 |
| 447770 | AB032417 | Hs.19545 | Frizzled, Fz, SS, TM, Frizzled, Fz, | frizzled (Drosophila) homolog 4 | 3.0 |
| 423826 | U20325 | Hs.1707 | SS, SS | cocaine-and amphetamine-regulated trans | 3.0 |
| 418838 | AW385224 | Hs.35198 | , SS, TM, Phosphodiest, | ectonucleotide pyrophosphatase/phosphodi | 3.0 |
| 442804 | AW300118 | Hs.131257 | , SS, TM, G-gamma | ESTs | 3.0 |
| 432284 | AA532807 | Hs.105822 | , SS, TM, pkinase, | ESTs | 3.0 |
| Table 20 shows 544 genes up-regulated in breast cancer compared to normal adult tissues that are likely to encode extracellular or cell-surface proteins. These were selected as for Table 19, except that the ratio was greater than or equal to 3.0, the “average” normal adult tissue level was set to the 85^{th} percentile value amongst 144 non-malignant tissues, and the 96^{th} percentile value amongst the 73 breast cancer specimens was greater than or equal 100 | | | | | |
| #units, and the predicted protein contained a structural domain that is indicative of extracellular localization (e.g. ig, fn3, egf, 7tm domains, signal sequences, transmembrane domains). The predicted protein domains are noted. | | | | | |

Pkey: Unique Eos probeset identifier number
ExAccn: Exemplar Accession number, Genbank accession number
UnigeneID: Unigene number
Pred. Prot. Domains: Predicted Protein Domains
Unigene Title: Unigene gene title
R1: Ratio of 93^{rd} percentile of tumor to 85^{th} percentile of normal body tissue

TABLE 20A
Table 20A shows the accession numbers for those
pkeys lacking unigeneID's for Table 20.
For each probeset, we have listed the gene cluster number
from which the oligonucleotides were designed.
Gene clusters were compiled using sequences derived
from Genbank ESTs and mRNAs. These sequences were
clustered based on sequence similarity using Clustering
and Alignment Tools (DoubleTwist, Oakland California).
The Genbank accession numbers for sequences comprising
each cluster are listed in the “Accession” column.

| Pkey | CAT number | Accessions |
|---|---|---|
| 410785 | 1221055_1 | AW803341 AW803265 AW803403 AW803466 |
| | | AW803402 AW803413 AW803268 |
| | | AW803396 AW803334 AW803355 |
| 411667 | 1253334_1 | BE160198 AW935898 T11520 AW935930 |
| | | AW856073 AW861034 |
| 418636 | 177402_1 | AW749855 AA225995 AW750208 AW750206 |
| 420854 | 197072_1 | AW296927 AI684514 AI263168 AA281079 |
| 422128 | 211994_1 | AW881145 AA490718 M85637 AA304575 |
| | | T06067 AA331991 |
| 423431 | 228162_1 | AA326062 AA325758 AW962182 |
| 423945 | 233566_1 | AA410943 AW948953 AA334202 AA332882 |
| 426878 | 273265_1 | BE069341 AW748403 AL044891 |
| | | AI908240 AA393080 |
| 433687 | 373061_1 | AA743991 AA604852 AW272737 |
| 447197 | 711623_1 | R36075 AI366546 R36167 |
| 451631 | 878098_1 | R00866 R01523 AI806815 |
| 456207 | 165078_−1 | AA193450 |
| 456592 | 202684_1 | R91600 T87079 AA291455 |

Pkey: Unique Eos probeset identifier number
CAT number: Gene cluster number
Accession: Genbank accession numbers

TABLE 20B
Table 20B shows the genomic positioning
for those pkeys lacking unigene ID's and
accession numbers in Table 20. For each predicted
exon, we have listed the genomic sequence source used
for prediction. Nucleotide locations of each predicted
exon are also listed.

| Pkey | Ref | Strand | Nt_position |
|---|---|---|---|
| 400608 | 9887666 | Minus | 96756-97558 |
| 400903 | 2911732 | Plus | 59112-59228 |
| 401045 | 8117619 | Plus | 90044-90184, 91111-91345 |
| 401093 | 8516137 | Minus | 22335-23166 |
| 401197 | 9719705 | Plus | 176341-176452 |
| 401747 | 9789672 | Minus | 118596-118816, |
| | | | 119119-119244, 119609-119761, |
| | | | 120422-120990, 130161-130381, |
| | | | 130468-130593, |
| | | | 131097-131258, 131866-131932, |
| | | | 132451-132575, 133580-134011 |
| 401780 | 7249190 | Minus | 28397-28617, |
| | | | 28920-29045, 29135-29296, |
| | | | 29411-29567, 29705-29787, |
| | | | 30224-30573 |
| 401781 | 7249190 | Minus | 83215-83435, |
| | | | 83531-83656, 83740-83901, |
| | | | 84237-84393, 84955-85037, |
| | | | 86290-86814 |
| 401866 | 8018106 | Plus | 73126-73623 |
| 402230 | 9966312 | Minus | 29782-29932 |
| 402408 | 9796239 | Minus | 110326-110491 |
| 402578 | 9884928 | Plus | 66350-66496 |
| 402606 | 9909429 | Minus | 81747-82094 |
| 402837 | 9369121 | Minus | 2013-2186, 9570-9758, 11136-11309, |
| | | | 19429-19677, 21210-21455, |
| | | | 23368-23562, |
| | | | 24342-24527, 29132-29320 |
| 403199 | 9958183 | Minus | 58895-59036, 66618-66789 |
| 403329 | 8516120 | Plus | 96450-96598 |
| 403593 | 6862650 | Minus | 62554-62712, 69449-69602 |
| 403943 | 7711864 | Plus | 100742-100904, 101322-101503 |
| 404091 | 7684554 | Minus | 82121-83229 |
| 404347 | 9838195 | Plus | 74493-74829 |
| 404826 | 6572184 | Plus | 47726-48046 |
| 405366 | 2182280 | Plus | 22478-22632 |
| 405654 | 4895155 | Minus | 53624-53759 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 21
210 GENES UP-REGULATED IN BREAST CANCER
COMPARED TO NORMAL ADULT TISSUES THAT ARE
LIKELY TO ENCODE EITHER ENZYMES OR PROTEINS
AMENABLE TO MODULATION BY SMALL MOLECULES

| | | Unigene | | | |
|---|---|---|---|---|---|
| Pkey | ExAccn | ID | Predicted Protein Domains | UnigeneTitle | R1 |
| 449746 | AI668594 | Hs.176588 | SS, p450 | ESTs, Weakly similar to CP4Y_HUMAN CYTOC | 65.7 |
| 400292 | AA250737 | Hs.72472 | death, ZU5, TM, Activin_recp, pkinase, | BMP-R1B | 55.9 |
| 424735 | U31875 | Hs.272499 | SS, TM | short-chain alcohol dehydrogenase family | 53.8 |
| 407178 | AA195651 | Hs.104106 | SS, Dihydroorotase, | ESTs | 39.3 |
| 408045 | AW138959 | Hs.245123 | Phosphodiest, Somatomedin_B, | ESTs | 34.9 |
| 450375 | AA009647 | Hs.8850 | SS, TM, disintegrin, Pep_M12B_propep, Reprol | a disintegrin and metalloproteinase doma | 25.7 |
| 429170 | NM_001394 | Hs.2359 | DSPc, Rhodanese, | dual specificity phosphatase 4 | 24.9 |
| 445730 | AI624342 | Hs.170042 | SS, TM, Cation_efflux | ESTs | 24.1 |
| 424634 | NM_003613 | Hs.151407 | ig, tsp_1, SS, AAA | cartilage intermediate layer protein, nu | 21.7 |
| 420757 | X78592 | Hs.99915 | hormone_rec, Androgen_recep, zf-C4, | androgen receptor (dihydrotestosterone r | 21.7 |
| 424399 | AI905687 | Hs.2533 | SS | aldehyde dehydrogenase 9 family, member | 20.3 |
| 447350 | AI375572 | Hs.172634 | pkinase, | ESTs | 19.2 |
| 456207 | AA193450 | | SS, TM, p450, p450 | gb: zr40e07.r1 Soares_NhHMPu_S1 Homo sapi | 18.3 |
| 456938 | X52509 | Hs.161640 | SS, TM, aminotran_1_2, Cadherin_C_term, | tyrosine aminotransferase | 18.1 |
| | | | cadh | | |
| 402578 | | | SS, p450, SS, TM, p450 | C1001134: gi\|2117372\|pir\|\|I65981 fatty ac | 17.8 |
| 425692 | D90041 | Hs.155956 | SS, Acetyltransf2, | N-acetyltransferase 1 (arylamine N-acety | 16.7 |
| 424001 | W67883 | Hs.137476 | pkinase, | paternally expressed 10 | 16.5 |
| 418007 | M13509 | Hs.83169 | SS, hemopexin, Peptidase_{—M10, SS, Peptidase}_{—} | matrix metalloproteinase 1 (MMP1; inters | 15.7 |
| 421727 | Y13153 | Hs.107318 | | kynurenine 3-monooxygenase (kynurenine 3 | 15.3 |
| 411869 | W20027 | Hs.23439 | SS, Peptidase_M1, | ESTs | 13.9 |
| 400289 | X07820 | Hs.2258 | hemopexin, Peptidase_M10, SS, Peptidase_M10 | matrix metalloproteinase 10 (MMP10; str | 13.5 |
| 443348 | AW873596 | Hs.182278 | SS, DENN | calmodulin 2 (phosphorylase kinase, delt | 13.0 |
| 424086 | AI351010 | Hs.102267 | SS, Lysyl_oxidase | lysyl oxidase | 12.8 |
| 400295 | W72838 | Hs.2533 | SS | aldehyde dehydrogenase 9 family, member | 12.7 |
| 408771 | AW732573 | Hs.47584 | TM, K_tetra, ion_trans, SS, TM, K_tetra, ion_t | potassium voltage-gated channel, delayed | 11.9 |
| 421155 | H87879 | Hs.102267 | SS, Lysyl_oxidase, Aldose_epim, Epimerase, S | lysyl oxidase | 11.8 |
| 424905 | NM_002497 | Hs.153704 | pkinase, SS, TM, pkinase, polyprenyL_synt, | NIMA (never in mitosis gene a)-related k | 11.7 |
| 438167 | R28363 | Hs.24286 | SS, TM, 7tm_1, p450, rrm | ESTs | 11.5 |
| 459583 | AI907673 | | pkinase, | gb: IL-BT152-080399-004 BT152 Homo sapien | 11.5 |
| 423945 | AA410943 | | death, ZU5, TM, Activin_recp, pkinase, | gb: zt32h03.r1 Soares ovary tumor NbHOT H | 11.4 |
| 445263 | H57646 | Hs.42586 | SS, Acyltransferase, | KIAA1560 protein | 11.2 |
| 423887 | AL080207 | Hs.134585 | SS, TM, BRCT, ank, ABC_tran, ABC_tran | DKFZP434G232 protein | 10.9 |
| 429859 | NM_007050 | Hs.225952 | SS, TM, Y_phosphatase, MAM, fn3, | protein tyrosine phosphatase, receptor t | 10.4 |
| 428368 | BE440042 | Hs.83326 | SS, Peptidase_M10, hemopexin, SS, Peptidase_{—} | matrix metalloproteinase 3 (stromelysin | 10.3 |
| 418912 | NM_000685 | Hs.89472 | SS, TM, 7tm_1, SS, TM, 7tm_1, | angiotensin receptor 1 | 10.3 |
| 451952 | AL120173 | Hs.301663 | SS, pkinase, | ESTs | 10.3 |
| 402408 | NA | | SS, carb_anhydrase | NM_030920*: Homo sapiens hypothetical pro | 9.8 |
| 415539 | AI733881 | Hs.72472 | death, ZU5, TM, Activin_recp, pkinase, | BMP-R1B | 9.4 |
| 406687 | M31126 | Hs.272620 | SS, Peptidase_M10, hemopexin, SS, Peptidase_{—} | matrix metalloproteinase 11 (MMP11; stro | 9.1 |
| 419948 | AB041035 | Hs.93847 | Ferric_reduct, TM, Ferric_reduct, | NM_016931: Homo sapiens NADPH oxidase 4 ( | 9.1 |
| 400285 | NA | | TM, ABC_tran, ABC_membrane, | Eos Control | 8.8 |
| 408380 | AF123050 | Hs.44532 | SS, TM, ubiquitin, 7tm_3, ANF_receptor, sushi | diubiquitin | 8.6 |
| 409203 | AA780473 | Hs.687 | SS, p450, SS, p450 | cytochrome P450, subfamily IVB, polypept | 8.3 |
| 424902 | NM_003866 | Hs.153687 | SS, SS | inositol polyphosphate-4-phosphatase, ty | 8.3 |
| 431725 | X65724 | Hs.2839 | SS, Cys_knot, SS | Norrie disease (pseudoglioma) | 8.3 |
| 418092 | R45154 | Hs.106604 | death, ZU5, pkinase, Activin_recp, | ESTs | 8.3 |
| 427811 | M81057 | Hs.180884 | SS, Zn_carbOpept, Propep_M14, SS, | carboxypeptidase B1 (tissue) | 8.2 |
| | | | Propep_M14 | | |
| 423554 | M90516 | Hs.1674 | GATase_2, SIS, | glutamine-fructose-6-phosphate transamin | 8.1 |
| 426261 | AW242243 | Hs.168670 | SS, TM, WD40, ubiquitin, E1-E2_ATPase, | peroxisomal famesylated protein | 7.8 |
| | | | Cation | | |
| 413374 | NM_001034 | Hs.75319 | SS | ribonucleotide reductase M2 polypeptide | 7.6 |
| 432677 | NM_004482 | Hs.278611 | SS, TM, Glycos_transf_2, Ricin_B_lectin, | UDP-N-acetyl-alpha-D-galactosamine: polyp | 7.6 |
| | | | DPP | | |
| 456986 | D38299 | Hs.170917 | SS, TM, 7tm_1, | prostaglandin E receptor 3 (subtype EP3) | 7.5 |
| 453331 | AI240665 | Hs.8895 | SS, TM, disintegrin, Pep_M12B_propep, Reprol | ESTs | 7.3 |
| 407721 | Y12735 | Hs.38018 | pkinase, | dual-specificity tyrosine-(Y)-phosphoryl | 7.2 |
| 418004 | U37519 | Hs.87539 | SS, aldedh, SS, aldedh, | aldehyde dehydrogenase 3 family, member | 7.2 |
| 410555 | U92649 | Hs.64311 | TM, disintegrin, Reprolysin, | a disintegrin and metalloproteinase doma | 7.1 |
| 443695 | AW204099 | Hs.337720 | | ESTs, Weakly similar to AF126780 1 retin | 6.9 |
| 423545 | AP000692 | Hs.129781 | GAF, PDEase | chromosome 21 open reading frame 5 | 6.8 |
| 401045 | | | ICE_p20, SS, ICE_p10, ICE_p20, | C11001883*: gi\|6753278\|ref\|NP_033938.1\| c | 6.7 |
| | | | ICE_p10, ICE_p | | |
| 442082 | R41823 | Hs.7413 | TM, EPH_Ibd, pkinase, SAM, fn3, | ESTs; calsyntenin-2 | 6.7 |
| 418836 | AI655499 | Hs.161712 | TM, Activin_recp, pkinase, death, ZU5, | ESTs | 6.6 |
| 404091 | NA | | TM, 7tm_3, ANF_receptor, | Target Exon | 6.6 |
| 450865 | AI248013 | Hs.106532 | zf-C2H2 | ESTs, Weakly similar to I38588 reverse t | 6.5 |
| 424085 | NM_002914 | Hs.139226 | SS, AAA, Viral_helicase1, mm, | replication factor C (activator 1) 2 (40 | 6.5 |
| 449650 | AF055575 | Hs.23838 | TM, ion_trans, SS, TM, ion_trans, | calcium channel, voltage-dependent, L ty | 6.4 |
| 432304 | AA932186 | Hs.69297 | TM, 7tm_1, | ESTs | 6.2 |
| 415669 | NM_005025 | Hs.78589 | SS, serpin, | serine (or cysteine) proteinase inhibito | 6.2 |
| 442942 | AW167087 | Hs.131562 | SS, ig, Sema, pkinase, | ESTs | 6.2 |
| 428795 | R45503 | Hs.97469 | SS, TM | ESTs, Highly similar to A39769 N-acetyll | 6.1 |
| 428479 | Y00272 | Hs.184572 | SS, pkinase, pkinase | cell division cycle 2, G1 to S and G2 to | 6.1 |
| 400300 | X03363 | | SS, TM, pkinase, Recep_L_domain, SH2, | HER2 receptor tyrosine kinase (c-erb-b2, | 6.1 |
| | | | PH, Furi | | |
| 447359 | NM_012093 | Hs.18268 | SS, adenylatekinase, | adenylate kinase 5 | 6.1 |
| 402230 | NA | | SS, TM, p450, | Target Exon | 6.1 |
| 424687 | J05070 | Hs.151738 | SS, Peptidase_M10, fn2, hemopexin, SS, | matrix metalloproteinase 9 (gelatinase B | 5.8 |
| | | | TM, Pep | | |
| 432328 | AI572739 | Hs.195471 | 6PF2K, PGAM, | 6-phosphofructo-2-kinase/fructose-2, 6-bi | 5.8 |
| 432239 | X81334 | Hs.2936 | SS, Peptidase_M10, hemopexin, SS, Peptidase_{—} | matrix metalloproteinase 13 (collagenase | 5.6 |
| 400286 | NA | | SS, TM, ABC_tran, ABC_membrane, SS | C16000922: gi\|7499103\|pir\|\|T20903 hypothe | 5.6 |
| 425247 | NM_005940 | Hs.155324 | SS, Peptidase_M10, hemopexin, SS | matrix metalloproteinase 11 (MMP11; stro | 5.4 |
| 434737 | AA828246 | Hs.291884 | UCH-1, pkinase, OPR, Rhodanese, AMP-binding, | ESTs | 5.4 |
| 439310 | AF086120 | Hs.102793 | SS, TM, UDPGT, casein_kappa | ESTs | 5.2 |
| 441111 | AI806867 | Hs.126594 | SS, TM, Phosphodiest, | ESTs | 5.2 |
| 452355 | N54926 | Hs.29202 | TM, 7tm_1, TM | G protein-coupled receptor 34 | 5.2 |
| 427711 | M31659 | Hs.180408 | SS | solute carrier family 25 (mitochondrial | 5.2 |
| 429353 | AL117406 | Hs.200102 | SS, TM, ABC_tran, ABC_membrane, | ATP-binding cassette transporter MRP8 | 5.1 |
| 425325 | X52730 | Hs.1892 | SS, NNMT_PNMT_TEMT, SS, | phenylethanolamine N-methyltransferase | 5.1 |
| | | | NNMT_PNMT_TEMT, STAR | | |
| 448706 | AW291095 | Hs.21814 | SS, TM, pkinase, | interleukin 20 receptor, alpha | 5.1 |
| 403593 | NA | | CIDE-N, pkinase | Target Exon | 5.1 |
| 432777 | AA564991 | Hs.269477 | alpha-amylase, | ESTs | 5.0 |
| 446232 | AI281848 | Hs.194691 | SS, TM, 7tm_3, Ribosomal_L13 | retinoic acid induced 3 | 4.9 |
| 411165 | NM_000169 | Hs.69089 | SS, Melibiase, BTK, PH, pkinase, SH2, | galactosidase, alpha | 4.9 |
| | | | SH3, Ribo | | |
| 414117 | W88559 | Hs.1787 | TM, ion_trans, K_tetra, | proteolipid protein 1 (Pelizaeus-Merzbac | 4.9 |
| 411096 | U80034 | Hs.68583 | Peptidase_M3, | mitochondrial intermediate peptidase | 4.9 |
| 450506 | NM_004460 | Hs.418 | SS, DPPIV_N_term, Peptidase_S9, SS, | fibroblast activation protein, alpha | 4.9 |
| | | | DPPIV_N_{—} | | |
| 417975 | AA641836 | Hs.30085 | SS, trypsin | hypothetical protein FLJ23186 | 4.9 |
| 447752 | M73700 | Hs.105938 | SS, transferrin, 7tm_1, transferrin, | lactotransferrin | 4.8 |
| 427122 | AW057736 | Hs.323910 | SS, TM, pkinase, Recep_L_domain, | HER2 receptor tyrosine kinase (c-erb-b2, | 4.8 |
| | | | SH2, PH, Furi | | |
| 400181 | NA | | SS, TM, 3Beta_HSD, | ENSP00000171555: CDNA FLJ10727 fis, clone | 4.6 |
| 452093 | AA447453 | Hs.27860 | SS, TM, 7tm_1, | Homo sapiens mRNA; cDNA DKFZp586M0723 (f | 4.6 |
| 435542 | AA687376 | Hs.269533 | SS, pkinase, RhoGEF, ig, PH, SH3, | ESTs | 4.6 |
| 417576 | AA339449 | Hs.82285 | AIRS, formyl_transf, GARS, SS, GARS, | phosphoribosylglycinamide formyltransfer | 4.6 |
| | | | AIRS, for | | |
| 446089 | AI860021 | Hs.270651 | pkinase | ESTs, Moderately similar to A47582 B-cel | 4.6 |
| 424420 | BE614743 | Hs.146688 | SS, TM, MAPEG, | prostaglandin E synthase | 4.5 |
| 452190 | H26735 | Hs.91668 | SS, TM, PH, SH2, Furin-like, pkinase, | Homo sapiens clone PP1498 unknown mRNA | 4.5 |
| | | | Recep_L_{—} | | |
| 419986 | AI345455 | Hs.78915 | pkinase, OPR, | GA-binding protein transcription factor, | 4.5 |
| 421582 | AI910275 | Hs.1406 | SS, trefoil, SS, TM, IdI_recept_a, SRCR, tryps | trefoil factor 1 (pS2) | 4.5 |
| 446733 | AA863360 | Hs.26040 | SS, TM, p450, | ESTs, Weakly similar to fatty acid omega | 4.4 |
| 453060 | AW294092 | Hs.21594 | SS, ras, Y_phosphatase, ras | hypothetical protein MGC15754 | 4.4 |
| 400205 | NA | | | NM_006265*: Homo sapiens RAD21 (S. pombe) | 4.4 |
| 420854 | AW296927 | | SS, TM, Peptidase_M1, | gb: UI-H-BW0-ajc-c-07-0-UI.s1 NCI_CGAP_Su | 4.3 |
| 432690 | AF181490 | Hs.278627 | SS, pyr_redox, SS, Ribosomal_L39 | prenylcysteine lyase | 4.3 |
| 441560 | F13386 | Hs.7888 | pkinase, | Homo sapiens clone 23736 mRNA sequence | 4.3 |
| 416445 | AL043004 | Hs.79337 | SS, pkinase, | KIAA0135 protein | 4.3 |
| 439024 | R96696 | Hs.35598 | SS, TM, trypsin, vwd, ig | ESTs | 4.3 |
| 432882 | NM_013257 | Hs.279696 | pkinase, pkinase_C, | serum/glucocorticoid regulated kinase-li | 4.2 |
| 447754 | AW073310 | Hs.163533 | pkinase, | Homo sapiens cDNA FLJ14142 fis, clone MA | 4.2 |
| 453775 | NM_002916 | Hs.35120 | SS, AAA, PI3_PI4_kinase, PI3Ka, | replication factor C (activator 1) 4 (37 | 4.2 |
| | | | PI3K_rbd, PI3 | | |
| 431657 | AI345227 | Hs.105448 | SS, TM, pkinase | ESTs, Weakly similar to B34087 hypotheti | 4.1 |
| 427899 | AA829286 | Hs.332053 | SS, SAA_proteins, ABC_membrane, | serum amyloid A1 | 4.1 |
| | | | ABC_tran, | | |
| 422938 | NM_001809 | Hs.1594 | SS, TM, thiolase, | centromere protein A (17 kD) | 4.1 |
| 418478 | U38945 | Hs.1174 | ank, ank | cyclin-dependent kinase inhibitor 2A (me | 4.1 |
| 406922 | S70284 | | SS, TM, Desaturase, SS | gb: stearoyl-CoA desaturase [human, adipo | 4.1 |
| 439285 | AL133916 | Hs.172572 | SS, ig, pkinase, LRRNT, LRRCT, | hypothetical protein FLJ20093 | 4.1 |
| 429922 | Z97630 | Hs.226117 | SS, TM, linker_histone, 7tm_1 | H1 histone family, member 0 | 4.1 |
| 420139 | NM_005357 | Hs.95351 | SS, TM, p450, | lipase, hormone-sensitive | 4.0 |
| 425071 | NM_013989 | Hs.154424 | SS, T4_deiodinase, T4_deiodinase, | deiodinase, iodothyronine, type II | 4.0 |
| 424511 | BE300512 | Hs.193557 | SS, Y_phosphatase, Band_41 | ESTs, Moderately similar to ALU7_HUMAN A | 4.0 |
| 428722 | U76456 | Hs.190787 | SS, TIMP, | tissue inhibitor of metalloproteinase 4 | 3.9 |
| 428330 | L22524 | Hs.2256 | SS, Peptidase_M10, SS, Peptidase_M10, | matrix metalloproteinase 7 (MMP7; uterin | 3.9 |
| | | | hemope | | |
| 414831 | M31158 | Hs.77439 | SS, cNMP_binding, Rlla, HMG_box | protein kinase, cAMP-dependent, regulato | 3.9 |
| 413589 | AW452631 | Hs.313803 | SS, abhydrolase | ESTs, Highly similar to AF157833 1 noncl | 3.8 |
| 418629 | BE247550 | Hs.86859 | SS, SH2, PH, SS, TM, PH, SH2, | growth factor receptor-bound protein 7 | 3.8 |
| | | | Furin-like, pkinas | | |
| 413453 | AA129640 | Hs.128065 | SS, Peptidase_C1, gpdh | ESTs | 3.8 |
| 403943 | | | p450, SS, p450 | C5000355: gi\|4503225\|ref\|NP_000765.1\|cyt | 3.8 |
| 444618 | AV653785 | Hs.173334 | | ELL-RELATED RNA POLYMERASE II, ELONGATIO | 3.8 |
| 408761 | AA057264 | Hs.238936 | SS, TM, 7tm_1, | ESTs, Weakly similar to (define not ava | 3.8 |
| 427809 | M26380 | Hs.180878 | SS, lipase, PLAT, Sec7, PH, | lipoprotein lipase | 3.7 |
| 418203 | X54942 | Hs.83758 | CKS, SS, CKS, | CDC28 protein kinase 2 | 3.7 |
| 454071 | AI041793 | Hs.42502 | TM, 7tm_1, | ESTs | 3.7 |
| 424676 | Y08565 | Hs.151678 | Glycos_transf_2, Ricin_B_lectin, SS, Glycos | UDP-N-acetyl-alpha-D-galactosamine: polyp | 3.7 |
| 457465 | AW301344 | Hs.122908 | SS, Pribosyltran, Sulfatase | DNA replication factor | 3.7 |
| 417601 | NM_014735 | Hs.82292 | PHD, pkinase, SS | KIAA0215 gene product | 3.7 |
| 446619 | AU076643 | Hs.313 | SS, TM, efhand, ion_trans | secreted phosphoprotein 1 (osteopontin, | 3.7 |
| 406625 | Y13647 | Hs.119597 | SS, TM, Desaturase, SS | stearoyl-CoA desaturase (delta-9-desatur | 3.6 |
| 428769 | AW207175 | Hs.106771 | SS, 7tm_1, SPRY, | ESTs | 3.6 |
| 426310 | NM_000909 | Hs.169266 | SS, TM, 7tm_1, | neuropeptide Y receptor Y1 | 3.6 |
| 417531 | NM_003157 | Hs.1087 | SS, pkinase, vwa, vwa, Glyco_transf_8 | serine/threonine kinase 2 | 3.6 |
| 444781 | NM_014400 | Hs.11950 | SS, PH, lactamase_B | GPI-anchored metastasis-associated prote | 3.6 |
| 431493 | AI791493 | Hs.129873 | SS, p450, p450 | ESTs, Weakly similar to A36036 cytochrom | 3.6 |
| 428966 | AF059214 | Hs.194687 | | cholesterol 25-hydroxylase | 3.6 |
| 414175 | AI308876 | Hs.103849 | TM, hemopexin, Peptidase_M10, | hypothetical protein DKFZp761D112 | 3.6 |
| | | | hemopexin, Pep | | |
| 455325 | AW895719 | | TM, ion_trans, K_tetra, | gb: QV4-NN0039-290300-154-f06 NN0039 Homo | 3.6 |
| 429597 | NM_003816 | Hs.2442 | | a disintegrin and metalloproteinase doma | 3.6 |
| 425320 | U29344 | Hs.83190 | Acyl_transf, adh_zinc, ketoacyl-synt, pp-bi | fatty acid synthase | 3.5 |
| 431854 | AA383550 | Hs.271699 | IMS, SS | polymerase (DNA directed) iota | 3.5 |
| 407104 | S57296 | Hs.323910 | SS, TM, SH2, PH, pkinase, | avian erythroblastic leukemia v | 3.5 |
| | | | Recep_L_domain, Furiv-erb-b2 | | |
| 449051 | AW961400 | Hs.333526 | SS | HER2 receptor tyrosine kinase (c-erb-b2, | 3.5 |
| 434398 | AA121098 | Hs.3838 | pkinase, POLO_box, SS, pkinase, POLO_box, | serum-inducible kinase | 3.5 |
| 454042 | H22570 | Hs.172572 | SS, ig, pkinase, LRRNT, LRRCT, | hypothetical protein FLJ20093 | 3.5 |
| 407756 | AA116021 | Hs.38260 | SS, UCH-1, UCH-2, SS, TM, G_glu_transpept | ubiquitin specific protease 18 | 3.5 |
| 401464 | AF039241 | Hs.9028 | Peptidase_M24, | histone deacetylase 5 | 3.5 |
| 412970 | AB026436 | Hs.177534 | DSPc, Myosin_tail, | dual specificity phosphatase 10 | 3.5 |
| 412049 | N53437 | Hs.18268 | SS, adenylatekinase, | adenylate kinase 5 | 3.5 |
| 425776 | U25128 | Hs.159499 | SS, TM, 7tm_2, SS, TM, 7tm_2 | parathyroid hormone receptor 2 | 3.4 |
| 407846 | AA426202 | Hs.40403 | TM, ABC_membrane, ABC_tran, | Cbp/p300-interacting transactivator, wit | 3.4 |
| | | | Ribosomal_S4e | | |
| 406925 | L34041 | Hs.9739 | SS, TM, transport_prot, SWIB, RhoGAP, | glycerol-3-phosphate dehydrogenase 1 (so | 3.4 |
| | | | DAG_PE- | | |
| 445873 | AA250970 | Hs.251946 | SS, rrm, PABP, pkinase, 14-3-3, rrm | poly(A)-binding protein, cytoplasmic 1-I | 3.4 |
| 418054 | NM_002318 | Hs.83354 | SS, TM, mito_carr, Lysyl_oxidase | lysyl oxidase-like 2 | 3.4 |
| 406815 | AA833930 | Hs.288036 | SS, IPPT, | tRNA isopentenylpyrophosphate transferas | 3.4 |
| 410530 | M25809 | Hs.64173 | ATP-synt_ab, SS, 7tm_1, ATP-synt_ab | ATPase, H transporting, lysosomal (vacuo | 3.4 |
| 407021 | U52077 | | | gb: Human mariner1 transposase gene, comp | 3.4 |
| 421168 | AF182277 | Hs.330780 | SS, p450, SS | cytochrome P450, subfamily IIB (phenobar | 3.4 |
| 431473 | AA825686 | Hs.321176 | SS | ESTs, Weakly similar to S65824 reverse t | 3.4 |
| 408101 | AW968504 | Hs.123073 | pkinase, | CDC2-related protein kinase 7 | 3.4 |
| 422083 | NM_001141 | Hs.111256 | lipoxygenase, PLAT, | arachidonate 15-lipoxygenase, second typ | 3.3 |
| 411393 | AW797437 | Hs.69771 | SS, sushi, trypsin, vwa, rrm, fibrinogen_C, fn | B-factor, properdin | 3.3 |
| 435767 | H73505 | Hs.117874 | SS, Peptidase_S8, P, Peptidase_S8, P | ESTs | 3.3 |
| 433068 | NM_006456 | Hs.288215 | SS, Pribosyltran, | sialyltransferase | 3.3 |
| 426928 | AF037062 | Hs.172914 | SS, adh_short, TGF-beta, TGFb_propeptide | retinol dehydrogenase 5 (11-cis and 9-ci | 3.3 |
| 414575 | H11257 | Hs.22968 | SS, pkinase, ig, | Homo sapiens clone IMAGE: 451939, mRNA se | 3.3 |
| 445941 | AI267371 | Hs.172636 | SS, SS, lipoxygenase, PLAT | ESTs | 3.3 |
| 444542 | AI161293 | Hs.280380 | SS, SS, Peptidase_M1, EGF, ig, lectin_c, sushi | aminopeptidase | 3.3 |
| 425741 | AF052152 | Hs.159412 | pkinase, | Homo sapiens clone 24628 mRNA sequence | 3.3 |
| 434228 | Z42047 | Hs.283978 | SS, TM, 7tm_1 | Homo sapiens PRO2751 mRNA, complete cds | 3.3 |
| 433264 | D85782 | Hs.3229 | | cysteine dioxygenase, type I | 3.3 |
| 400419 | AF084545 | | SS, Peptidase_M1, | Target | 3.3 |
| 439750 | AL359053 | Hs.57664 | TM, integrin_B, Ricin_B_lectin, rrm | Homo sapiens mRNA full length insert cDN | 3.3 |
| 417757 | R19897 | Hs.106604 | death, ZU5, pkinase, Activin_recp, | ESTs | 3.3 |
| 452194 | AI694413 | Hs.332649 | SS, TM, 7tm_3, ANF_receptor, sushi | olfactory receptor, family 2, subfamily | 3.2 |
| 421458 | NM_003654 | Hs.104576 | SS | carbohydrate (keratan sulfate Gal-6) sul | 3.2 |
| 443767 | BE562136 | Hs.9736 | SS, PCI, RasGEF, hormone_rec, zf-C4, | proteasome (prosome, macropain) 26S subu | 3.2 |
| 422648 | D86983 | Hs.118893 | peroxidase, LRRCT, | Melanoma associated gene | 3.2 |
| 423431 | AA326062 | | SS, p450, p450 | gb: EST29171 Cerebellum II Homo sapiens c | 3.2 |
| 451264 | AI768235 | | SS, Trehalase | gb: wg82g08.x1 Soares_NSF_F8_9W_OT_PA_P_S | 3.2 |
| 452110 | T47667 | Hs.28005 | SS, TM, Activin_recp, pkinase | Homo sapiens cDNA FLJ11309 fis, clone PL | 3.2 |
| 439963 | AW247529 | Hs.6793 | TM, p450, Ets | platelet-activating factor acetylhydrola | 3.2 |
| 453941 | U39817 | Hs.36820 | SS, DEAD, HRDC, helicase_C, | Bloom syndrome | 3.1 |
| 406664 | L34041 | Hs.9739 | SS, TM, transport_prot, SWIB, RhoGAP, | glycerol-3-phosphate dehydrogenase 1 (so | 3.1 |
| | | | DAG_PE- | | |
| 453487 | R31770 | Hs.23540 | TM, 7tm_1, | ESTs | 3.1 |
| 420911 | U77413 | Hs.100293 | | O-linked N-acetylglucosamine (GIcNAc) tr | 3.1 |
| 443171 | BE281128 | Hs.9030 | SS, TM, 7tm_1, rrm, SS | TONDU | 3.1 |
| 452256 | AK000933 | Hs.28661 | TM, GDI, 7tm_1, | Homo sapiens cDNA FLJ10071 fis, clone HE | 3.1 |
| 432201 | AI538613 | Hs.298241 | SS, TM, trypsin, SS, TM, trefoil, trypsin, tref | Transmembrane protease, serine 3 | 3.1 |
| 419150 | T29618 | Hs.89640 | TM, pkinase, fn3, | TEK tyrosine kinase, endothelial (venous | 3.1 |
| 444443 | AI149286 | Hs.55099 | SS | rab6 GTPase activating protein (GAP and | 3.1 |
| 426283 | NM_003937 | Hs.169139 | | kynureninase (L-kynurenine hydrolase) | 3.1 |
| 436291 | BE568452 | Hs.5101 | SS, abhydrolase, | protein regulator of cytokinesis 1 | 3.1 |
| 450223 | AA418204 | Hs.241493 | SS, pro_isomerase, | natural killer-tumor recognition sequenc | 3.1 |
| 424269 | AW137691 | Hs.199754 | SS, TM, 7tm_2, GPS | ESTs | 3.1 |
| 448105 | AW591433 | Hs.298241 | SS, TM, trefoil, trypsin, trefoil | Transmembrane protease, serine 3 | 3.0 |
| 452560 | BE077084 | Hs.336432 | SS, rrm, zf-RanBP, pkinase, C2, pkinase_C, | ESTs | 3.0 |
| | | | DAG | | |
| Table 21 shows 210 genes up-regulated in breast cancer compared to normal adult tissues that are likely to encode either enzymes or proteins amenable to modulation by small molecules. These were selected as for Table 19, except that the ratio was greater than or equal to 3.0, the “average” normal adult tissue level was set to the 85^{th} percentile value amongst 144 non-malignant tissues, and the 96^{th} percentile value amongst 73 breast cancers was | | | | | |
| #greater than or equal 80 units, and the predicted protein contained a structural domain that is indicative of enzymatic function or of being modulatable by small molecules (e.g. pkinase, peptidase, phosphatase, ATPase, or ion_transporter domains). The predicted protein domains are noted. | | | | | |

Pkey: Unique Eos probeset identifier number
ExAccn: Exemplar Accession number, Genbank accession number
UnigeneID: Unigene number
Unigene Title: Unigene gene title
R1: Ratio of 93rd percentile tumor to 85^{th} percentile of normal body tissue

TABLE 21A
Table 21 A shows the accession numbers for
those pkeys lacking unigeneID's for Table 21.
For each probeset, we have listed the gene cluster
number from which the oligonucleotides were designed.
Gene clusters were compiled using sequences derived
from Genbank ESTs and mRNAs. These sequences were
clustered based on sequence similarity using Clustering
and Alignment Tools (DoubleTwist, Oakland California).
The Genbank accession numbers for sequences comprising
each cluster are listed in the “Accession” column.

| Pkey | CAT number | Accessions |
|---|---|---|
| 420854 | 197072_1 | AW296927 AI684514 AI263168 AA281079 |
| 423431 | 228162_1 | AA326062 AA325758 AW962182 |
| 423945 | 233566_1 | AA410943 AW948953 AA334202 AA332882 |
| 451264 | 863988_1 | AI768235 R31400 H29082 H23107 |
| 455325 | 1279475_1 | AW895719 N31451 N41451 |
| 456207 | 165078_−1 | AA193450 |

Pkey: Unique Eos probeset identifier number
CAT number: Gene cluster number
Accession: Genbank accession numbers

TABLE 21B
Table 21B shows the genomic positioning for
those pkeys lacking unigene ID's and accession
numbers in Table 21. For each predicted exon,
we have listed the genomic sequence source used
for prediction. Nucleotide locations of each
predicted exon are also listed.

| Pkey | Ref | Strand | Nt_position |
|---|---|---|---|
| 401045 | 8117619 | Plus | 90044-90184, 91111-91345 |
| 402230 | 9966312 | Minus | 29782-29932 |
| 402408 | 9796239 | Minus | 110326-110491 |
| 402578 | 9884928 | Plus | 66350-66496 |
| 403593 | 6862650 | Minus | 62554-62712, 69449-69602 |
| 403943 | 7711864 | Plus | 100742-100904, 101322-101503 |
| 404091 | 7684554 | Minus | 82121-83229 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 22
739 GENES UP-REGULATED IN BREAST CANCER
COMPARED TO NORMAL ADULT BREAST

| Pkey | ExAccn | UnigeneID | UnigeneTitle | R1 |
|---|---|---|---|---|
| 400292 | AA250737 | Hs.72472 | BMP-R1B | 51.5 |
| 424735 | U31875 | Hs.272499 | short-chain alcohol dehydrogenase family | 38.3 |
| 400297 | AI127076 | Hs.334473 | hypothetical protein DKFZp564O1278 | 29.9 |
| 431448 | AL137517 | Hs.334473 | hypothetical protein DKFZp564O1278 | 26.9 |
| 451110 | AI955040 | Hs.265398 | ESTs, Weakly similar to transformation-r | 25.8 |
| 431211 | M86849 | Hs.323733 | gap junction protein, beta 2, 26 kD (conn | 23.2 |
| 418203 | X54942 | Hs.83758 | CDC28 protein kinase 2 | 22.6 |
| 407980 | AA046309 | | gb: zf12f01.s1 Soares_fetal_heart_NbHH19W | 19.8 |
| 414646 | AA353776 | Hs.901 | CD48 antigen (B-cell membrane protein) | 18.9 |
| 446921 | AB012113 | Hs.16530 | small inducible cytokine subfamily A (Cy | 18.0 |
| 409041 | AB033025 | Hs.50081 | KIAA1199 protein | 17.6 |
| 412140 | AA219691 | Hs.73625 | RAB6 interacting, kinesin-like (rabkines | 17.6 |
| 407824 | AA147884 | Hs.9812 | Homo sapiens cDNA FLJ14388 fis, clone HE | 17.1 |
| 453160 | AI263307 | Hs.239884 | H2B histone family, member L | 17.0 |
| 407137 | T97307 | | gb: ye53h05.s1 Soares fetal liver spleen | 16.1 |
| 425692 | D90041 | Hs.155956 | N-acetyltransferase 1 (arylamine N-acety | 16.1 |
| 438533 | AI440266 | Hs.170673 | ESTs, Weakly similar to T24832 hypotheti | 16.0 |
| 428227 | AA321649 | Hs.2248 | small inducible cytokine subfamily B (CX | 15.5 |
| 444342 | NM_014398 | Hs.10887 | similar to lysosome-associated membrane | 15.1 |
| 422505 | AL120862 | Hs.124165 | programmed cell death 9 (PDCD9) | 14.9 |
| 430515 | AA746503 | Hs.283313 | ESTs | 14.7 |
| 417308 | H60720 | Hs.81892 | KIAA0101 gene product | 14.4 |
| 452744 | AI267652 | Hs.30504 | Homo sapiens mRNA; cDNA DKFZp434E082 (fr | 14.4 |
| 412446 | AI768015 | Hs.92127 | ESTs | 14.2 |
| 415539 | AI733881 | Hs.72472 | BMP-R1B | 14.1 |
| 435496 | AW840171 | Hs.265398 | ESTs, Weakly similar to transformation-r | 13.8 |
| 438209 | AL120659 | Hs.6111 | aryl-hydrocarbon receptor nuclear transl | 13.8 |
| 400205 | NA | | NM_006265*: Homo sapiens RAD21 (S. pombe) | 13.5 |
| 430965 | AA489732 | Hs.154918 | ESTs | 13.4 |
| 415263 | AA948033 | Hs.130853 | ESTs | 13.3 |
| 451952 | AL120173 | Hs.301663 | ESTs | 13.2 |
| 449722 | BE280074 | Hs.23960 | cyclin B1 | 13.2 |
| 406685 | M18728 | | gb: Human nonspecific crossreacting antig | 13.0 |
| 406690 | M29540 | Hs.220529 | carcinoembryonic antigen-related cell ad | 12.8 |
| 429925 | NM_000786 | Hs.226213 | cytochrome P450, 51 (lanosterol 14-alpha | 12.8 |
| 416498 | U33632 | Hs.79351 | potassium channel, subfamily K, member 1 | 12.7 |
| 432378 | AI493046 | Hs.146133 | ESTs | 12.5 |
| 441377 | BE218239 | Hs.202656 | ESTs | 12.5 |
| 456207 | AA193450 | | gb: zr40e07.r1 Soares_NhHMPu_S1 Homo sapi | 12.4 |
| 422805 | AA436989 | Hs.121017 | H2A histone family, member A | 12.2 |
| 407811 | AW190902 | Hs.40098 | cysteine knot superfamily 1, BMP antagon | 12.2 |
| 407178 | AA195651 | Hs.104106 | ESTs | 12.2 |
| 420931 | AF044197 | Hs.100431 | small inducible cytokine B subfamily (Cy | 12.1 |
| 421727 | Y13153 | Hs.107318 | kynurenine 3-monooxygenase (kynurenine 3 | 12.0 |
| 434408 | AI031771 | Hs.132586 | ESTs | 12.0 |
| 446591 | H44186 | Hs.15456 | PDZ domain containing 1 | 11.9 |
| 431385 | BE178536 | Hs.11090 | membrane-spanning 4-domains, subfamily A | 11.8 |
| 443348 | AW873596 | Hs.182278 | calmodulin 2 (phosphorylase kinase, delt | 11.7 |
| 416602 | NM_006159 | Hs.79389 | nel (chicken)-like 2 | 11.7 |
| 433365 | AF026944 | Hs.293797 | ESTs | 11.6 |
| 437866 | AA156781 | Hs.74170 | metallothionein 1E (functional) | 11.5 |
| 412472 | AW975398 | Hs.293836 | ESTs | 11.4 |
| 416030 | H15261 | Hs.21948 | ESTs | 11.3 |
| 439979 | AW600291 | Hs.6823 | hypothetical protein FLJ10430 | 11.3 |
| 420757 | X78592 | Hs.99915 | androgen receptor (dihydrotestosterone r | 11.3 |
| 411598 | BE336654 | Hs.70937 | H3 histone family, member A | 11.2 |
| 423600 | AI633559 | Hs.310359 | ESTs | 11.2 |
| 430770 | AA765694 | Hs.123296 | ESTs | 11.0 |
| 421037 | AI684808 | Hs.197653 | programmed cell death 9 (PDCD9) | 10.9 |
| 452461 | N78223 | Hs.108106 | transcription factor | 10.7 |
| 409269 | AA576953 | Hs.22972 | hypothetical protein FLJ13352 | 10.6 |
| 417791 | AW965339 | Hs.111471 | ESTs | 10.6 |
| 447268 | AI370413 | Hs.36563 | hypothetical protein FLJ22418 | 10.4 |
| 424001 | W67883 | Hs.137476 | paternally expressed 10 | 10.4 |
| 447342 | AI199268 | Hs.19322 | Homo sapiens, Similar to RIKEN cDNA 2010 | 10.4 |
| 424905 | NM_002497 | Hs.153704 | NIMA (never in mitosis gene a)-related k | 10.1 |
| 453619 | H87648 | Hs.33922 | Homo sapiens, clone MGC: 9084, mRNA, comp | 10.1 |
| 442942 | AW167087 | Hs.131562 | ESTs | 10.1 |
| 434377 | AW137148 | Hs.306593 | Homo sapiens cDNA FLJ11382 fis, clone HE | 10.1 |
| 427217 | AA399272 | Hs.144341 | ESTs | 10.1 |
| 445730 | AI624342 | Hs.170042 | ESTs | 10.0 |
| 432887 | AI926047 | Hs.162859 | ESTs | 10.0 |
| 452243 | AL355715 | Hs.28555 | programmed cell death 9 | 9.9 |
| 424590 | AW966399 | Hs.46821 | hypothetical protein FLJ20086 | 9.9 |
| 432169 | Y00971 | Hs.2910 | phosphoribosyl pyrophosphate synthetase | 9.9 |
| 438950 | H23789 | Hs.144530 | EST | 9.9 |
| 418836 | AI655499 | Hs.161712 | ESTs | 9.8 |
| 430291 | AV660345 | Hs.238126 | CGI-49 protein | 9.8 |
| 444665 | BE613126 | Hs.47783 | B aggressive lymphoma gene | 9.7 |
| 407377 | C16391 | | gb: C16391 Clontech human aorta polyA mRN | 9.7 |
| 445413 | AA151342 | Hs.12677 | CGI-147 protein | 9.7 |
| 443462 | AI064690 | Hs.171176 | ESTs | 9.7 |
| 442145 | AI022650 | Hs.8117 | erbb2-interacting protein ERBIN | 9.7 |
| 435570 | AF212222 | Hs.177812 | uncharacterized bone marrow protein BM04 | 9.7 |
| 439820 | AL360204 | Hs.283853 | Homo sapiens mRNA full length insert cDN | 9.6 |
| 428966 | AF059214 | Hs.194687 | cholesterol 25-hydroxylase | 9.6 |
| 449448 | D60730 | Hs.57471 | ESTs | 9.6 |
| 433929 | AI375499 | Hs.27379 | ESTs | 9.5 |
| 432731 | R31178 | Hs.287820 | fibronectin 1 | 9.5 |
| 411815 | AA156679 | Hs.125790 | leucine-rich repeat-containing 2 | 9.3 |
| 415385 | R17798 | Hs.7535 | COBW-like protein | 9.3 |
| 422026 | U80736 | Hs.110826 | trinucleotide repeat containing 9 | 9.2 |
| 432596 | AJ224741 | Hs.278461 | matrilin 3 | 9.2 |
| 439451 | AF086270 | Hs.278554 | heterochromatin-like protein 1 | 9.2 |
| 423945 | AA410943 | | gb: zt32h03.r1 Soares ovary tumor NbHOT H | 9.1 |
| 442432 | BE093589 | Hs.38178 | hypothetical protein FLJ23468 | 9.1 |
| 446715 | AI337735 | Hs.173919 | ESTs, Moderately similar to ZN91_HUMAN Z | 9.0 |
| 408771 | AW732573 | Hs.47584 | potassium voltage-gated channel, delayed | 9.0 |
| 437021 | AI076089 | Hs.292239 | ESTs | 9.0 |
| 428479 | Y00272 | Hs.184572 | cell division cycle 2, G1 to S and G2 to | 8.9 |
| 428839 | AI767756 | Hs.82302 | Homo sapiens cDNA FLJ14814 fis, clone NT | 8.9 |
| 402408 | NA | | NM_030920*: Homo sapiens hypothetical pro | 8.9 |
| 418601 | A279490 | Hs.86368 | calmegin | 8.8 |
| 426327 | W03242 | Hs.44898 | Homo sapiens clone TCCCTA00151 mRNA sequ | 8.8 |
| 419519 | AI198719 | Hs.176376 | ESTs | 8.8 |
| 440621 | AW296024 | Hs.150434 | ESTs | 8.8 |
| 446142 | AI754693 | Hs.145968 | ESTs | 8.8 |
| 418196 | AI745649 | Hs.26549 | KIAA1708 protein | 8.7 |
| 447178 | AW594641 | Hs192417 | ESTs | 8.7 |
| 427585 | D31152 | Hs.179729 | collagen, type X, alpha 1 (Schmid metaph | 8.6 |
| 415857 | AA866115 | Hs.127797 | Homo sapiens cDNA FLJ11381 fis, clone HE | 8.6 |
| 435061 | AI651474 | Hs.163944 | ESTs | 8.6 |
| 431374 | BE258532 | Hs.251871 | CTP synthase | 8.4 |
| 417866 | AW067903 | Hs.82772 | collagen, type XI, alpha 1 | 8.4 |
| 437211 | AA382207 | Hs.5509 | ecotropic viral integration site 2B | 8.3 |
| 437751 | AA767373 | Hs.35669 | ESTs, Moderately similar to ALU1_HUMAN A | 8.3 |
| 423887 | AL080207 | Hs.134585 | DKFZP434G232 protein | 8.2 |
| 440941 | BE268362 | Hs.7535 | COBW-like protein | 8.2 |
| 429859 | NM_007050 | Hs.225952 | protein tyrosine phosphatase, receptor t | 8.2 |
| 410193 | AJ132592 | Hs.59757 | zinc finger protein 281 | 8.2 |
| 431725 | X65724 | Hs.2839 | Norrie disease (pseudoglioma) | 8.1 |
| 446258 | AI283476 | Hs.263478 | ESTs | 8.1 |
| 416747 | AW876523 | Hs.15929 | hypothetical protein FLJ12910 | 8.1 |
| 434424 | AI811202 | Hs.325335 | Homo sapiens cDNA: FLJ23523 fis, clone L | 8.1 |
| 421650 | AA781795 | Hs.122587 | ESTs | 8.0 |
| 429534 | AW976987 | Hs.163327 | ESTs, Weakly similar to 2109260A B cell | 8.0 |
| 457465 | AW301344 | Hs.122908 | DNA replication factor | 8.0 |
| 427961 | AW293165 | Hs.143134 | ESTs | 8.0 |
| 436481 | AA379597 | Hs.5199 | HSPC150 protein similar to ubiquitin-con | 8.0 |
| 418216 | AA662240 | Hs.283099 | AF15q14 protein | 8.0 |
| 418250 | U29926 | Hs.83918 | adenosine monophosphate deaminase (isofo | 7.9 |
| 400285 | NA | | Eos Control | 7.9 |
| 401464 | AF039241 | Hs.9028 | histone deacetylase 5 | 7.9 |
| 407242 | M18728 | | gb: Human nonspecific crossreacting antig | 7.8 |
| 422232 | D43945 | Hs.113274 | transcription factor EC | 7.8 |
| 454024 | AA993527 | Hs.293907 | hypothetical protein FLJ23403 | 7.8 |
| 444542 | AI161293 | Hs.280380 | aminopeptidase | 7.8 |
| 436396 | AI683487 | Hs.152213 | wingless-type MMTV integration site fami | 7.7 |
| 437204 | AL110216 | Hs.12285 | ESTs, Weakly similar to I55214 salivary | 7.6 |
| 408805 | H69912 | Hs.48269 | vaccinia related kinase 1 | 7.6 |
| 437207 | T27503 | Hs.15929 | hypothetical protein FLJ12910 | 7.6 |
| 442818 | AK001741 | Hs.8739 | hypothetical protein FLJ10879 | 7.6 |
| 426283 | NM_003937 | Hs.169139 | kynureninase (L-kynurenine hydrolase) | 7.5 |
| 424687 | J05070 | Hs.151738 | matrix metalloproteinase 9 (gelatinase B | 7.5 |
| 446315 | NM_016293 | Hs.14770 | bridging integrator 2 | 7.5 |
| 433426 | H69125 | Hs.133525 | ESTs | 7.5 |
| 406639 | M97711 | | gb: Human T-cell receptor (V beta 18.1, J | 7.5 |
| 420077 | AW512260 | Hs.87767 | ESTs | 7.4 |
| 457332 | AA961694 | Hs.105187 | kinesin protein 9 gene | 7.4 |
| 422938 | NM_001809 | Hs.1594 | centromere protein A (17 kD) | 7.4 |
| 447555 | AI391662 | Hs.160963 | Homo sapiens, clone MGC: 12318, mRNA, com | 7.4 |
| 444618 | AV653785 | Hs.173334 | ELL-RELATED RNA POLYMERASE II, ELONGATIO | 7.3 |
| 410361 | BE391804 | Hs.62661 | guanylate binding protein 1, interferon- | 7.3 |
| 400268 | NA | | NM_003292: Homo sapiens translocated prom | 7.3 |
| 439509 | AF086332 | Hs.58314 | ESTs | 7.3 |
| 407771 | AL138272 | Hs.62713 | ESTs | 7.3 |
| 407202 | N58172 | Hs.109370 | ESTs | 7.3 |
| 433096 | AU076803 | Hs.282975 | carboxylesterase 2 (intestine, liver) | 7.2 |
| 422094 | AF129535 | Hs.272027 | F-box only protein 5 | 7.1 |
| 430832 | AI073913 | Hs.100686 | ESTs, Weakly similar to JE0350 Anterior | 7.1 |
| 430287 | AW182459 | Hs.125759 | ESTs, Weakly similar to LEU5_HUMAN LEUKE | 7.0 |
| 423739 | AA398155 | Hs.97600 | ESTs | 7.0 |
| 448212 | AI475858 | | gb: tc87d07.x1 NCI_CGAP_CLL1 Homo sapiens | 7.0 |
| 407277 | AW170035 | Hs.326736 | Homo sapiens breast cancer antigen NY-BR | 7.0 |
| 454440 | BE062906 | Hs.28338 | KIAA1546 protein | 7.0 |
| 444783 | AK001468 | Hs.62180 | anillin (Drosophila Scraps homolog), act | 7.0 |
| 421373 | AA808229 | Hs.167771 | ESTs | 6.9 |
| 431960 | AW241821 | Hs.301927 | c6.1A | 6.9 |
| 424704 | AI263293 | Hs.152096 | cytochrome P450, subfamily IIJ (arachido | 6.8 |
| 449517 | AW500106 | Hs.23643 | serine/threonine protein kinase MASK | 6.8 |
| 439840 | AW449211 | Hs.105445 | GDNF family receptor alpha 1 | 6.8 |
| 414080 | AA135257 | Hs.47783 | B aggressive lymphoma gene | 6.8 |
| 441243 | AI767056 | Hs.193002 | ESTs | 6.7 |
| 408380 | AF123050 | Hs.44532 | diubiquitin | 6.7 |
| 422956 | BE545072 | Hs.122579 | hypothetical protein FLJ10461 | 6.7 |
| 446651 | AA393907 | Hs.97179 | ESTs | 6.7 |
| 419839 | U24577 | Hs.93304 | phospholipase A2, group VII (platelet-ac | 6.7 |
| 437740 | AA810265 | Hs.122915 | ESTs | 6.7 |
| 421582 | AI910275 | Hs.1406 | trefoil factor 1 (pS2) | 6.7 |
| 427356 | AW023482 | Hs.97849 | ESTs | 6.6 |
| 429597 | NM_003816 | Hs.2442 | a disintegrin and metalloproteinase doma | 6.6 |
| 422634 | NM_016010 | Hs.118821 | CGI-62 protein | 6.6 |
| 421072 | AI215069 | Hs.89113 | ESTs | 6.5 |
| 427718 | AI798680 | Hs.25933 | ESTs | 6.5 |
| 411000 | N40449 | Hs.201619 | ESTs, Weakly similar to S38383 SEB4B pro | 6.5 |
| 449343 | AI151418 | Hs.272458 | protein phosphatase 3 (formerly 2B), cat | 6.4 |
| 409757 | NM_001898 | Hs.123114 | cystatin SN | 6.4 |
| 447164 | AF026941 | Hs.17518 | Homo sapiens cig5 mRNA, partial sequence | 6.4 |
| 456938 | X52509 | Hs.161640 | tyrosine aminotransferase | 6.4 |
| 418848 | AI820961 | Hs.193465 | ESTs | 6.4 |
| 424902 | NM_003866 | Hs.153687 | inositol polyphosphate-4-phosphatase, ty | 6.4 |
| 452838 | U65011 | Hs.30743 | preferentially expressed antigen in mela | 6.4 |
| 439452 | AA918317 | Hs.57987 | B-cell CLL/lymphoma 11B (zinc finger pro | 6.4 |
| 407266 | AJ235664 | | gb: Homo sapiens mRNA for immunoglobulin | 6.3 |
| 411078 | AI222020 | Hs.182364 | CocoaCrisp | 6.3 |
| 433001 | AF217513 | Hs.279905 | clone HQ0310 PRO0310p1 | 6.3 |
| 434340 | AI193043 | Hs.128685 | ESTs, Weakly similar to T17226 hypotheti | 6.2 |
| 429503 | AA394183 | Hs.26873 | ESTs | 6.2 |
| 402578 | | | C1001134: gi\|2117372\|pir\|\|I165981 fatty ac | 6.2 |
| 409646 | AW161391 | Hs.709 | deoxycytidine kinase | 6.1 |
| 430447 | W17064 | Hs.332848 | SWI/SNF related, matrix associated, acti | 6.1 |
| 432415 | T16971 | Hs.289014 | ESTs, Weakly similar to A43932 mucin 2 p | 6.1 |
| 443709 | AI082692 | Hs.134662 | ESTs | 6.1 |
| 420929 | AI694143 | Hs.296251 | programmed cell death 4 | 6.1 |
| 428368 | BE440042 | Hs.83326 | matrix metalloproteinase 3 (stromelysin | 6.1 |
| 428248 | AI126772 | Hs.40479 | ESTs | 6.0 |
| 420344 | BE463721 | Hs.97101 | putative G protein-coupled receptor | 6.0 |
| 453392 | U23752 | Hs.32964 | SRY (sex determining region Y)-box 11 | 6.0 |
| 425397 | J04088 | Hs.156346 | topoisomerase (DNA) II alpha (170 kD) | 6.0 |
| 418007 | M13509 | Hs.83169 | matrix metalloproteinase 1 (MMP1; inters | 6.0 |
| 428585 | AB007863 | Hs.185140 | KIAA0403 protein | 6.0 |
| 437608 | AA761605 | Hs.292308 | ESTs, Weakly similar to ALU1_HUMAN ALU S | 6.0 |
| 427408 | AA583206 | Hs.2156 | RAR-related orphan receptor A | 6.0 |
| 406687 | M31126 | Hs.272620 | matrix metalloproteinase 11 (MMP11; stro | 6.0 |
| 418092 | R45154 | Hs.106604 | ESTs | 6.0 |
| 447051 | AW139130 | Hs.160951 | ESTs, Weakly similar to Con1 [H. sapiens] | 6.0 |
| 441233 | AA972965 | Hs.135568 | ESTs | 6.0 |
| 432239 | X81334 | Hs.2936 | matrix metalloproteinase 13 (collagenase | 6.0 |
| 435106 | AA100847 | Hs.193380 | ESTs, Highly similar to AF174600 1 F-box | 5.9 |
| 435525 | AI831297 | Hs.123310 | ESTs | 5.9 |
| 458809 | AW972512 | Hs.20985 | sin3-associated polypeptide, 30 kD | 5.9 |
| 410785 | AW803341 | | gb: IL2-UM0079-090300-050-D03 UM0079 Homo | 5.9 |
| 422576 | BE548555 | Hs.118554 | CGI-83 protein | 5.9 |
| 451398 | AI793124 | Hs.144479 | ESTs | 5.9 |
| 441881 | AW968904 | Hs.179566 | hypothetical protein FLJ22624 | 5.8 |
| 412022 | AI005043 | Hs.24143 | Wiskott-Aldrich syndrome protein interac | 5.8 |
| 416636 | N32536 | Hs.42645 | solute carrier family 16 (monocarboxylic | 5.8 |
| 447350 | AI375572 | Hs.172634 | ESTs | 5.8 |
| 434094 | AA305599 | Hs.238205 | hypothetical protein PRO2013 | 5.8 |
| 409151 | AA306105 | Hs.50785 | SEC22, vesicle trafficking protein (S. c | 5.8 |
| 448807 | AI571940 | Hs.7549 | ESTs | 5.8 |
| 452281 | T93500 | Hs.28792 | Homo sapiens cDNA FLJ11041 fis, clone PL | 5.8 |
| 421281 | AI299139 | Hs.17517 | ESTs | 5.8 |
| 430361 | AI033965 | Hs.239926 | sterol-C4-methyl oxidase-like | 5.8 |
| 400289 | X07820 | Hs.2258 | matrix metalloproteinase 10 (MMP10; str | 5.7 |
| 440527 | AV657117 | Hs.184164 | ESTs, Moderately similar to S65657 alpha | 5.7 |
| 434674 | AA831879 | Hs.136985 | ESTs | 5.7 |
| 426320 | W47595 | Hs.169300 | transforming growth factor, beta 2 | 5.7 |
| 452401 | NM_007115 | Hs.29352 | tumor necrosis factor, alpha-induced pro | 5.7 |
| 448663 | BE614599 | Hs.106823 | hypothetical protein MGC14797 | 5.7 |
| 438199 | AW016531 | Hs.122147 | ESTs | 5.7 |
| 446203 | Z47553 | Hs.14286 | flavin containing monooxygenase 5 | 5.7 |
| 428336 | AA503115 | Hs.183752 | microseminoprotein, beta- | 5.6 |
| 430379 | AF134149 | Hs.240395 | potassium channel, subfamily K, member 6 | 5.6 |
| 422835 | BE218705 | Hs.121378 | metallothionein-like 5, testis-specific | 5.6 |
| 444758 | AL044878 | Hs.11899 | 3-hydroxy-3-methylglutaryl-Coenzyme A re | 5.6 |
| 443426 | AF098158 | Hs.9329 | chromosome 20 open reading frame 1 | 5.6 |
| 400301 | X03635 | Hs.1657 | estrogen receptor 1 | 5.6 |
| 447078 | AW885727 | Hs.301570 | ESTs | 5.6 |
| 432015 | AL157504 | Hs.159115 | Homo sapiens mRNA; cDNA DKFZp586O0724 (f | 5.5 |
| 438691 | AA906288 | Hs.212184 | ESTs | 5.5 |
| 439809 | R41396 | Hs.101774 | hypothetical protein FLJ23045 | 5.5 |
| 415786 | AW419196 | Hs.257924 | hypothetical protein FLJ13782 | 5.5 |
| 456373 | BE247706 | Hs.89751 | membrane-spanning 4-domains, subfamily A | 5.5 |
| 401645 | NA | | C16001440*: gi\|12330704\|gb\|AAG52890.1\|AF3 | 5.5 |
| 437967 | BE277414 | Hs.5947 | mel transforming oncogene (derived from | 5.5 |
| 445885 | AI734009 | Hs.127699 | KIAA1603 protein | 5.4 |
| 439138 | AI742605 | Hs.193696 | ESTs | 5.4 |
| 440270 | NM_015986 | Hs.7120 | cytokine receptor-like molecule 9 | 5.4 |
| 437536 | X91221 | Hs.144465 | ESTs | 5.4 |
| 438167 | R28363 | Hs.24286 | ESTs | 5.4 |
| 452741 | BE392914 | Hs.30503 | Homo sapiens cDNA FLJ11344 fis, clone PL | 5.4 |
| 426214 | H59846 | Hs.128355 | ESTs, Moderately similar to ALU7_HUMAN A | 5.4 |
| 413554 | AA319146 | Hs.75426 | secretogranin II (chromogranin C) | 5.4 |
| 422867 | L32137 | Hs.1584 | cartilage oligomeric matrix protein (COM | 5.4 |
| 434263 | N34895 | Hs.44648 | ESTs | 5.4 |
| 446382 | AW205168 | Hs.150823 | ESTs | 5.4 |
| 422406 | AF025441 | Hs.116206 | Opa-interacting protein 5 | 5.3 |
| 438321 | AA576635 | Hs.6153 | CGI-48 protein | 5.3 |
| 418310 | AA814100 | Hs.86693 | ESTs | 5.3 |
| 419625 | U91616 | Hs.91640 | nuclear factor of kappa light polypeptid | 5.3 |
| 450701 | H39960 | Hs.288467 | Homo sapiens cDNA FLJ12280 fis, clone MA | 5.3 |
| 445900 | AF070526 | Hs.13429 | Homo sapiens clone 24787 mRNA sequence | 5.2 |
| 449051 | AW961400 | Hs.333526 | HER2 receptor tyrosine kinase (c-erb-b2, | 5.2 |
| 418758 | AW959311 | Hs.172012 | hypothetical protein DKFZp434J037 | 5.2 |
| 431070 | AW408164 | Hs.249184 | transcription factor 19 (SC1) | 5.2 |
| 417079 | U65590 | Hs.81134 | interleukin 1 receptor antagonist | 5.2 |
| 421928 | AF013758 | Hs.109643 | polyadenylate binding protein-interactin | 5.2 |
| 428804 | AK000713 | Hs.193736 | hypothetical protein FLJ20706 | 5.2 |
| 427427 | AF077345 | Hs.177936 | ESTs | 5.2 |
| 403485 | | | C3001813*: gi\|12737279\|ref\|XP_012163.1\|k | 5.2 |
| 422168 | AA586894 | Hs.112408 | S100 calcium-binding protein A7 (psorias | 5.1 |
| 421937 | AI878857 | Hs.109706 | hematological and neurological expressed | 5.1 |
| 426752 | X69490 | Hs.172004 | titin | 5.1 |
| 453310 | X70697 | Hs.553 | solute carrier family 6 (neurotransmitte | 5.1 |
| 423198 | M81933 | Hs.1634 | cell division cycle 25A | 5.1 |
| 412281 | AI810054 | Hs.14119 | ESTs | 5.1 |
| 447513 | AW955776 | Hs.313500 | ESTs, Moderately similar to ALU7_HUMAN A | 5.1 |
| 453931 | AL121278 | Hs.25144 | ESTs | 5.1 |
| 404347 | | | Target Exon | 5.1 |
| 431808 | M30703 | Hs.270833 | amphiregulin (schwannoma-derived growth | 5.1 |
| 429113 | D28235 | Hs.196384 | prostaglandin-endoperoxide synthase 2 (p | 5.1 |
| 436291 | BE568452 | Hs.5101 | protein regulator of cytokinesis 1 | 5.1 |
| 450603 | R43646 | Hs.12422 | ESTs | 5.1 |
| 434725 | AK000796 | Hs.4104 | hypothetical protein | 5.0 |
| 435981 | H74319 | Hs.188620 | ESTs | 5.0 |
| 407376 | AA993138 | Hs.142287 | ESTs, Weakly similar to ALUF_HUMAN !!!! | 5.0 |
| 431689 | AA305688 | Hs.267695 | UDP-Gal: betaGlcNAc beta 1,3-galactosyltr | 5.0 |
| 405348 | NA | | C7001664: gi\|12698061\|dbj\|BAB21849.1\| (AB | 5.0 |
| 436196 | AK001084 | Hs.333498 | Homo sapiens cDNA FLJ10222 fis, clone HE | 5.0 |
| 437065 | AL036450 | Hs.103238 | ESTs | 5.0 |
| 410196 | AI936442 | Hs.59838 | hypothetical protein FLJ10808 | 5.0 |
| 429412 | NM_006235 | Hs.2407 | POU domain, class 2, associating factor | 5.0 |
| 446619 | AU076643 | Hs.313 | secreted phosphoprotein 1 (osteopontin, | 4.9 |
| 403329 | NA | | Target Exon | 4.9 |
| 442875 | BE623003 | Hs.23625 | Homo sapiens clone TCCCTA00142 mRNA sequ | 4.9 |
| 442441 | AI820662 | Hs.129598 | ESTs | 4.9 |
| 430375 | AW371048 | Hs.93758 | H4 histone family, member H | 4.9 |
| 424128 | AW966163 | | gb: EST378236 MAGE resequences, MAGI Homo | 4.9 |
| 408873 | AL046017 | Hs.182278 | calmodulin 2 (phosphorylase kinase, delt | 4.9 |
| 407910 | AA650274 | Hs.41296 | fibronectin leucine rich transmembrane p | 4.9 |
| 432606 | NM_002104 | Hs.3066 | granzyme K (serine protease, granzyme 3; | 4.9 |
| 453204 | R10799 | Hs.191990 | ESTs | 4.8 |
| 452020 | AA722012 | Hs.255757 | ESTs, Weakly similar to AT2A_HUMAN POTEN | 4.8 |
| 449048 | Z45051 | Hs.22920 | similar to S68401 (cattle) glucose induc | 4.8 |
| 408369 | R38438 | Hs.182575 | solute carrier family 15 (H??? transport | 4.8 |
| 431645 | AF078849 | Hs.266483 | dynein light chain-A | 4.8 |
| 423575 | C18863 | Hs.163443 | Homo sapiens cDNA FLJ11576 fis, clone HE | 4.8 |
| 444246 | H93281 | Hs.10710 | hypothetical protein FLJ20417 | 4.8 |
| 421524 | AA312082 | Hs.105445 | GDNF family receptor alpha 1 | 4.8 |
| 452827 | AI571835 | Hs.55468 | ESTs | 4.8 |
| 414222 | AL135173 | Hs.878 | sorbitol dehydrogenase | 4.8 |
| 456086 | AL161999 | Hs.77324 | eukaryotic translation termination facto | 4.8 |
| 419078 | M93119 | Hs.89584 | insulinoma-associated 1 | 4.8 |
| 418973 | AA233056 | Hs.191518 | ESTs | 4.8 |
| 447033 | AI357412 | Hs.157601 | ESTs | 4.8 |
| 451621 | AI879148 | Hs.26770 | fatty acid binding protein 7, brain | 4.7 |
| 419968 | X04430 | Hs.93913 | interleukin 6 (interferon, beta 2) | 4.7 |
| 424326 | NM_014479 | Hs.145296 | disintegrin protease | 4.7 |
| 431585 | BE242803 | Hs.262823 | hypothetical protein FLJ10326 | 4.7 |
| 429294 | AA095971 | Hs.198793 | Homo sapiens cDNA: FLJ22463 fis, clone H | 4.7 |
| 416814 | AW192307 | Hs.80042 | dolichyl-P-Glc: Man9GlcNAc2-PP-dolichylgl | 4.7 |
| 439897 | NM_015310 | Hs.6763 | KIAA0942 protein | 4.7 |
| 429687 | AI675749 | Hs.211608 | nucleoporin 153 kD | 4.7 |
| 422880 | AF228704 | Hs.121524 | glutathione reductase | 4.7 |
| 405801 | | | NM_000390: Homo sapiens choroideremia (Ra | 4.6 |
| 432435 | BE218886 | Hs.282070 | ESTs | 4.6 |
| 439544 | W26354 | Hs.28891 | hypothetical protein FLJ11360; artemis p | 4.6 |
| 425354 | U62027 | Hs.155935 | complement component 3a receptor 1 | 4.6 |
| 436027 | AI864053 | Hs.39972 | ESTs, Weakly similar to I38588 reverse t | 4.6 |
| 424623 | AW963062 | Hs.337404 | ESTs | 4.6 |
| 403366 | NA | | Target Exon | 4.6 |
| 402542 | | | Target Exon | 4.6 |
| 450193 | AI916071 | Hs.15607 | Homo sapiens Fanconi anemia complementat | 4.6 |
| 411678 | AI907114 | Hs.71465 | squalene epoxidase | 4.6 |
| 456844 | AI264155 | Hs.152981 | CDP-diacylglycerol synthase (phosphatida | 4.6 |
| 448072 | AI459306 | Hs.24908 | ESTs | 4.5 |
| 408045 | AW138959 | Hs.245123 | ESTs | 4.5 |
| 423782 | AI472209 | Hs.323117 | ESTs | 4.5 |
| 447388 | AW630534 | Hs.76277 | Homo sapiens, clone MGC: 9381, mRNA, comp | 4.5 |
| 448140 | AF146761 | Hs.20450 | BCM-like membrane protein precursor | 4.5 |
| 452561 | AI692181 | Hs.49169 | KIAA1634 protein | 4.5 |
| 425331 | AW962128 | | gb: EST374201 MAGE resequences, MAGG Homo | 4.5 |
| 428801 | AW277121 | Hs.254881 | ESTs | 4.5 |
| 428500 | AI815395 | Hs.184641 | fatty acid desaturase 2 | 4.5 |
| 426075 | AW513691 | Hs.270149 | ESTs, Weakly similar to 2109260A B cell | 4.4 |
| 437259 | AI377755 | Hs.120695 | ESTs | 4.4 |
| 400409 | AF153341 | Hs.283954 | Homo sapiens winged helix/forkhead trans | 4.4 |
| 412863 | AA121673 | Hs.59757 | zinc finger protein 281 | 4.4 |
| 426989 | AI815206 | Hs.99395 | ESTs | 4.4 |
| 401866 | | | Target Exon | 4.4 |
| 418819 | AA228776 | Hs.191721 | ESTs | 4.4 |
| 406348 | | | Target Exon | 4.4 |
| 412138 | AW895387 | | gb: QV4-NN0038-300300-157-c10 NN0038 Homo | 4.4 |
| 428550 | AW297880 | Hs.98661 | ESTs | 4.4 |
| 411743 | AW862214 | | gb: QV4-CT0361-301299-074-b05 CT0361 Homo | 4.4 |
| 429966 | BE081342 | Hs.283037 | HSPC039 protein | 4.4 |
| 423291 | NM_004129 | Hs.126590 | guanylate cyclase 1, soluble, beta 2 | 4.4 |
| 423456 | AL110151 | Hs.128797 | DKFZP586D0824 protein | 4.4 |
| 452190 | H26735 | Hs.91668 | Homo sapiens clone PP1498 unknown mRNA | 4.4 |
| 424871 | NM_004525 | Hs.153595 | low density lipoprotein-related protein | 4.3 |
| 429575 | AA706003 | Hs.99387 | ESTs | 4.3 |
| 429922 | Z97630 | Hs.226117 | H1 histone family, member 0 | 4.3 |
| 421379 | Y15221 | Hs.103982 | small inducible cytokine subfamily B (Cy | 4.3 |
| 400300 | X03363 | | HER2 receptor tyrosine kinase (c-erb-b2, | 4.3 |
| 437258 | AL041243 | Hs.174104 | ESTs | 4.3 |
| 446595 | T57448 | Hs.15467 | hypothetical protein FLJ20725 | 4.3 |
| 403011 | NA | | ENSP00000215330*: Probable serine/threoni | 4.3 |
| 419055 | AI365384 | Hs.11571 | Homo sapiens cDNA FLJ11570 fis, clone HE | 4.3 |
| 418661 | NM_001949 | Hs.1189 | E2F transcription factor 3 | 4.3 |
| 407786 | AA687538 | Hs.38972 | tetraspan 1 | 4.3 |
| 429183 | AB014604 | Hs.197955 | KIAA0704 protein | 4.3 |
| 442914 | AW188551 | Hs.99519 | hypothetical protein FLJ14007 | 4.3 |
| 441029 | AI091795 | Hs.179246 | ESTs | 4.3 |
| 452194 | AI694413 | Hs.332649 | olfactory receptor, family 2, subfamily | 4.3 |
| 414821 | M63835 | Hs.77424 | Fc fragment of lgG, high affinity la, re | 4.2 |
| 410102 | AW248508 | Hs.279727 | Homo sapiens cDNA FLJ14035 fis, clone HE | 4.2 |
| 452110 | T47667 | Hs.28005 | Homo sapiens cDNA FLJ11309 fis, clone PL | 4.2 |
| 442007 | AA301116 | Hs.142838 | nucleolar phosphoprotein Nopp34 | 4.2 |
| 417318 | AW953937 | Hs.12891 | ESTs | 4.2 |
| 431818 | AW510444 | Hs.191705 | ESTs, Weakly similar to T47184 hypotheti | 4.2 |
| 443646 | AI085198 | Hs.164226 | ESTs | 4.2 |
| 419169 | AW851980 | Hs.262346 | ESTs, Weakly similar to S72482 hypotheti | 4.2 |
| 446839 | BE091926 | Hs.16244 | mitotic spindle coiled-coil related prot | 4.2 |
| 423242 | AL039402 | Hs.125783 | DEME-6 protein | 4.2 |
| 432116 | AA902953 | Hs.308538 | ESTs | 4.2 |
| 409038 | T97490 | Hs.50002 | small inducible cytokine subfamily A (Cy | 4.2 |
| 445625 | BE246743 | Hs.288529 | hypothetical protein FLJ22635 | 4.2 |
| 425139 | AW630488 | Hs.325820 | protease, serine, 23 | 4.2 |
| 447397 | BE247676 | Hs.18442 | E-1 enzyme | 4.2 |
| 410166 | AK001376 | Hs.59346 | hypothetical protein FLJ10514 | 4.1 |
| 437295 | AW779318 | Hs.88417 | ESTs | 4.1 |
| 430486 | BE062109 | Hs.241551 | chloride channel, calcium activated, fam | 4.1 |
| 441790 | AW294909 | Hs.132208 | ESTs | 4.1 |
| 410129 | BE244074 | Hs.58831 | regulator of Fas-induced apoptosis | 4.1 |
| 427521 | AW973352 | Hs.290585 | ESTs | 4.1 |
| 425247 | NM_005940 | Hs.155324 | matrix metalloproteinase 11 (MMP11; stro | 4.1 |
| 412886 | AF041163 | Hs.74647 | Human T-cell receptor active alpha-chain | 4.1 |
| 441153 | BE562826 | | gb: 601336534F1 NIH_MGC_44 Homo sapiens c | 4.1 |
| 444301 | AK000136 | Hs.10760 | asporin (LRR class 1) | 4.1 |
| 426711 | AA383471 | Hs.180669 | conserved gene amplified in osteosarcoma | 4.1 |
| 405850 | NA | | Target Exon | 4.1 |
| 440283 | AI732892 | Hs.190489 | ESTs | 4.0 |
| 432441 | AW292425 | Hs.163484 | ESTs | 4.0 |
| 400284 | NA | | estrogen receptor 1 | 4.0 |
| 417341 | N91453 | Hs.102987 | ESTs | 4.0 |
| 429732 | U20158 | Hs.2488 | lymphocyte cytosolic protein 2 (SH2 doma | 4.0 |
| 411393 | AW797437 | Hs.69771 | B-factor, properdin | 4.0 |
| 425704 | U79293 | Hs.159264 | Human clone 23948 mRNA sequence | 4.0 |
| 419594 | AA013051 | Hs.91417 | topoisomerase (DNA) II binding protein | 4.0 |
| 419092 | J05581 | Hs.89603 | mucin 1, transmembrane | 4.0 |
| 443147 | AI034351 | Hs.19030 | ESTs | 4.0 |
| 408633 | AW963372 | Hs.46677 | PRO2000 protein | 4.0 |
| 433404 | T32982 | Hs.102720 | ESTs | 4.0 |
| 421506 | BE302796 | Hs.105097 | thymidine kinase 1, soluble | 4.0 |
| 417900 | BE250127 | Hs.82906 | CDC20 (cell division cycle 20, S. cerevi | 3.9 |
| 414602 | AW630088 | Hs.76550 | Homo sapiens mRNA; cDNA DKFZp564B1264 (f | 3.9 |
| 413762 | AW411479 | Hs.848 | FK506-binding protein 4 (59 kD) | 3.9 |
| 404580 | | | NM_014112*: Homo sapiens trichorhinophala | 3.9 |
| 452046 | AB018345 | Hs.27657 | KIAA0802 protein | 3.9 |
| 459587 | AA031956 | | gb: zk15e04.s1 Soares_pregnant_uterus_NbH | 3.9 |
| 416658 | U03272 | Hs.79432 | fibrillin 2 (congenital contractural ara | 3.9 |
| 426647 | AA243464 | Hs.294101 | pre-B-cell leukemia transcription factor | 3.9 |
| 429353 | AL117406 | Hs.200102 | ATP-binding cassette transporter MRP8 | 3.9 |
| 419038 | AW134924 | Hs.190325 | ESTs | 3.9 |
| 418918 | X07871 | Hs.89476 | CD2 antigen (p50), sheep red blood cell | 3.9 |
| 421977 | W94197 | Hs.110165 | ribosomal protein L26 homolog | 3.9 |
| 442567 | AI201183 | Hs.130251 | ESTs | 3.9 |
| 421168 | AF182277 | Hs.330780 | cytochrome P450, subfamily IIB (phenobar | 3.9 |
| 431701 | AW935490 | Hs.14658 | Human chromosome 5q13.1 clone 5G8 mRNA | 3.9 |
| 418526 | BE019020 | Hs.85838 | solute carrier family 16 (monocarboxylic | 3.9 |
| 414998 | NM_002543 | Hs.77729 | oxidised low density lipoprotein (lectin | 3.9 |
| 422790 | AA809875 | Hs.25933 | ESTs | 3.9 |
| 419741 | NM_007019 | Hs.93002 | ubiquitin carrier protein E2-C | 3.9 |
| 430017 | AA263172 | Hs.35 | protein tyrosine phosphatase, non-recept | 3.9 |
| 458814 | AI498957 | Hs.170861 | ESTs, Weakly similar to Z195_HUMAN ZINC | 3.8 |
| 428514 | AW236861 | Hs.193139 | ESTs | 3.8 |
| 434521 | NM_002267 | Hs.3886 | karyopherin alpha 3 (importin alpha 4) | 3.8 |
| 409425 | U40462 | Hs.54452 | zinc finger protein, subfamily 1A, 1 (lk | 3.8 |
| 439560 | BE565647 | Hs.74899 | hypothetical protein FLJ12820 | 3.8 |
| 424028 | AF055084 | Hs.153692 | Homo sapiens cDNA FLJ14354 fis, clone Y7 | 3.8 |
| 400021 | | | AFFX control - HUMISGF3A/M97935_MA | 3.8 |
| 453403 | BE466639 | Hs.61779 | Homo sapiens cDNA FLJ13591 fis, clone PL | 3.8 |
| 445941 | AI267371 | Hs.172636 | ESTs | 3.8 |
| 434378 | AA631739 | Hs.335440 | EST | 3.8 |
| 429220 | AW207206 | Hs.136319 | ESTs | 3.8 |
| 439176 | AI446444 | Hs.190394 | ESTs, Weakly similar to B28096 line-1 pr | 3.8 |
| 401045 | | | C11001883*: gi\|6753278\|ref\|NP_033938.1\| c | 3.8 |
| 430178 | AW449612 | Hs.152475 | ESTs | 3.8 |
| 423397 | NM_001838 | Hs.1652 | chemokine (C-C motif) receptor 7 | 3.8 |
| 447630 | AI660149 | Hs.44865 | lymphoid enhancer-binding factor 1 | 3.8 |
| 436391 | AJ227892 | Hs.146274 | ESTs | 3.8 |
| 413011 | AW068115 | Hs.821 | biglycan | 3.8 |
| 422121 | AI767949 | Hs.179833 | ESTs | 3.8 |
| 452268 | NM_003512 | Hs.28777 | H2A histone family, member L | 3.8 |
| 427811 | M81057 | Hs.180884 | carboxypeptidase B1 (tissue) | 3.8 |
| 415579 | AA165232 | Hs.222069 | ESTs | 3.8 |
| 437330 | AL353944 | Hs.50115 | Homo sapiens mRNA; cDNA DKFZp761J1112 (f | 3.8 |
| 427122 | AW057736 | Hs.323910 | HER2 receptor tyrosine kinase (c-erb-b2, | 3.7 |
| 400286 | NA | | C16000922: gi\|7499103\|pir\|\|T20903 hypothe | 3.7 |
| 420281 | AI623693 | Hs.191533 | ESTs | 3.7 |
| 419926 | AW900992 | Hs.93796 | DKFZP586D2223 protein | 3.7 |
| 417541 | AI992191 | Hs.180040 | hypothetical protein FLJ22439 | 3.7 |
| 426172 | AA371307 | Hs.125056 | ESTs | 3.7 |
| 429638 | AI916662 | Hs.211577 | kinectin 1 (kinesin receptor) | 3.7 |
| 457001 | J03258 | Hs.2062 | vitamin D (1,25-dihydroxyvitamin D3) re | 3.7 |
| 424109 | AW406878 | | gb: UI-HF-BL0-adg-g-06-0-UI.r1 NIH_MGC_37 | 3.7 |
| 417022 | NM_014737 | Hs.80905 | Ras association (RalGDS/AF-6) domain fam | 3.7 |
| 436222 | AI208737 | Hs.122810 | Homo sapiens cDNA FLJ11489 fis, clone HE | 3.7 |
| 430448 | AI633553 | Hs.13303 | Homo sapiens cDNA: FLJ21784 fis, clone H | 3.7 |
| 432729 | AK000292 | Hs.278732 | hypothetical protein FLJ20285 | 3.7 |
| 413916 | N49813 | Hs.75615 | apolipoprotein C-II | 3.7 |
| 421662 | NM_014141 | Hs.106552 | cell recognition molecule Caspr2 | 3.7 |
| 441633 | AW958544 | Hs.112242 | normal mucosa of esophagus specific 1 | 3.7 |
| 408761 | AA057264 | Hs.238936 | ESTs, Weakly similar to (defline not ava | 3.7 |
| 406153 | | | Target Exon | 3.7 |
| 445563 | AW873606 | Hs.149006 | ESTs | 3.7 |
| 453464 | AI884911 | Hs.32989 | receptor (calcitonin) activity modifying | 3.7 |
| 448918 | AB011152 | Hs.22572 | KIAA0580 protein | 3.7 |
| 413936 | AF113676 | Hs.297681 | serine (or cysteine) proteinase inhibito | 3.6 |
| 448069 | U76248 | Hs.20191 | seven in absentia (Drosophila) homolog 2 | 3.6 |
| 453313 | BE005771 | Hs.153746 | hypothetical protein FLJ22490 | 3.6 |
| 425234 | AW152225 | Hs.165909 | ESTs, Weakly similar to I38022 hypotheti | 3.6 |
| 419941 | X98654 | Hs.93837 | phosphatidylinositol transfer protein, m | 3.6 |
| 402397 | AF188625 | Hs.189507 | phospholipase A2, group IID | 3.6 |
| 430378 | Z29572 | Hs.2556 | tumor necrosis factor receptor superfami | 3.6 |
| 448106 | AI800470 | Hs.171941 | ESTs | 3.6 |
| 426431 | NM_000458 | Hs.169853 | transcription factor 2, hepatic; LF-B3; | 3.6 |
| 431843 | AA516420 | Hs.183526 | ESTs, Weakly similar to I38022 hypotheti | 3.6 |
| 426878 | BE069341 | | gb: QV3-BT0381-270100-073-c08 BT0381 Homo | 3.6 |
| 434061 | AW024973 | Hs.283675 | NPD009 protein | 3.6 |
| 445292 | AV653264 | Hs.13982 | Homo sapiens cDNA FLJ14666 fis, clone NT | 3.6 |
| 452101 | T60298 | Hs.10844 | Homo sapiens cDNA FLJ14476 fis, clone MA | 3.6 |
| 427581 | NM_014788 | Hs.179703 | KIAA0129 gene product | 3.6 |
| 409047 | AW961434 | Hs.31539 | ESTs | 3.6 |
| 416820 | NM_000402 | Hs.80206 | glucose-6-phosphate dehydrogenase | 3.6 |
| 410386 | W26187 | Hs.3327 | Homo sapiens cDNA: FLJ22219 fis, clone H | 3.6 |
| 440516 | S42303 | Hs.161 | cadherin 2, type 1, N-cadherin (neuronal | 3.6 |
| 434360 | AW015415 | Hs.127780 | ESTs | 3.6 |
| 428970 | BE276891 | Hs.194691 | retinoic acid induced 3 | 3.6 |
| 415079 | R43179 | Hs.22895 | hypothetical protein FLJ23548 | 3.6 |
| 409619 | AK001015 | Hs.55220 | BCL2-associated athanogene 2 | 3.6 |
| 430044 | AA464510 | Hs.152812 | ESTs | 3.6 |
| 430829 | AW451999 | Hs.194024 | ESTs | 3.6 |
| 434224 | AA380731 | Hs.84 | interleukin 2 receptor, gamma (severe co | 3.6 |
| 439247 | AF088020 | Hs.46767 | EST | 3.6 |
| 431542 | H63010 | Hs.5740 | ESTs | 3.5 |
| 430713 | AA351647 | Hs.2642 | eukaryotic translation elongation factor | 3.5 |
| 434988 | AI418055 | Hs.161160 | ESTs | 3.5 |
| 437748 | AF234882 | Hs.5814 | suppression of tumorigenicity 7 | 3.5 |
| 418322 | AA284166 | Hs.84113 | cyclin-dependent kinase inhibitor 3 (CDK | 3.5 |
| 439569 | AW602166 | Hs.222399 | CEGP1 protein | 3.5 |
| 459583 | AI907673 | | gb: IL-BT152-080399-004 BT152 Homo sapien | 3.5 |
| 403212 | | | NM_019595: Homo sapiens intersectin 2 (IT | 3.5 |
| 409099 | AK000725 | Hs.50579 | hypothetical protein FLJ20718 | 3.5 |
| 453968 | AA847843 | Hs.62711 | Homo sapiens, clone IMAGE: 3351295, mRNA | 3.5 |
| 436338 | W92147 | Hs.118394 | ESTs | 3.5 |
| 422890 | Z43784 | Hs.75893 | ankyrin 3, node of Ranvier (ankyrin G) | 3.5 |
| 442295 | AI827248 | Hs.224398 | Homo sapiens cDNA FLJ11469 fis, clone HE | 3.5 |
| 417975 | AA641836 | Hs.30085 | hypothetical protein FLJ23186 | 3.5 |
| 433730 | AK002135 | Hs.3542 | hypothetical protein FLJ11273 | 3.5 |
| 439926 | AW014875 | Hs.137007 | ESTs | 3.5 |
| 445873 | AA250970 | Hs.251946 | poly(A)-binding protein, cytoplasmic 1-I | 3.4 |
| 410153 | BE311926 | Hs.15830 | hypothetical protein FLJ12691 | 3.4 |
| 422128 | AW881145 | | gb: QV0-OT0033-010400-182-a07 OT0033 Homo | 3.4 |
| 414921 | BE390551 | Hs.77628 | steroidogenic acute regulatory protein r | 3.4 |
| 441134 | W29092 | Hs.7678 | cellular retinoic acid-binding protein 1 | 3.4 |
| 444564 | AI167877 | Hs.143716 | ESTs | 3.4 |
| 402470 | NA | | Target Exon | 3.4 |
| 418120 | AA213437 | Hs.192249 | ESTs | 3.4 |
| 422414 | AW875237 | Hs.13701 | ESTs | 3.4 |
| 433345 | AI681545 | Hs.152982 | hypothetical protein FLJ13117 | 3.4 |
| 409213 | U61412 | Hs.51133 | PTK6 protein tyrosine kinase 6 | 3.4 |
| 422611 | AA158177 | Hs.118722 | fucosyltransferase 8 (alpha (1,6) fucosy | 3.4 |
| 423554 | M90516 | Hs.1674 | glutamine-fructose-6-phosphate transamin | 3.4 |
| 442619 | AA447492 | Hs.20183 | ESTs, Weakly similar to AF164793 1 prote | 3.4 |
| 402359 | NA | | C19001991*: gi\|12656111\|gb\|AAK00751.1\|AF2 | 3.4 |
| 439398 | AA284267 | Hs.221504 | ESTs | 3.4 |
| 415208 | F01020 | Hs.172004 | titin | 3.4 |
| 452853 | AA812633 | Hs.10845 | ESTs | 3.4 |
| 429345 | R11141 | Hs.199695 | hypothetical protein | 3.4 |
| 449027 | AJ271216 | Hs.22880 | dipeptidylpeptidase III | 3.4 |
| 412115 | AK001763 | Hs.73239 | hypothetical protein FLJ10901 | 3.4 |
| 432180 | Y18418 | Hs.272822 | RuvB (E coli homolog)-like 1 | 3.4 |
| 428977 | AK001404 | Hs.194698 | cyclin B2 | 3.4 |
| 431611 | U58766 | Hs.264428 | tissue specific transplantation antigen | 3.4 |
| 418286 | AA622528 | Hs.319825 | Homo sapiens, clone IMAGE: 3616574, mRNA, | 3.4 |
| 436895 | AF037335 | Hs.5338 | carbonic anhydrase XII (tumor antigen H | 3.4 |
| 443378 | AW392550 | Hs.9280 | proteasome (prosome, macropain) subunit, | 3.4 |
| 428450 | NM_014791 | Hs.184339 | KIAA0175 gene product | 3.3 |
| 449571 | AW016812 | Hs.200266 | ESTs | 3.3 |
| 412777 | AI335773 | Hs.270123 | ESTs | 3.3 |
| 420542 | NM_000505 | Hs.1321 | coagulation factor XII (Hageman factor) | 3.3 |
| 412754 | AW160375 | Hs.74565 | amyloid beta (A4) precursor-like protein | 3.3 |
| 418327 | U70370 | Hs.84136 | paired-like homeodomain transcription fa | 3.3 |
| 449065 | AI627393 | Hs.258998 | ESTs, Weakly similar to high mobility gr | 3.3 |
| 425999 | AW513051 | Hs.332981 | ESTs, Weakly similar to I38022 hypotheti | 3.3 |
| 430280 | AA361258 | Hs.237868 | interleukin 7 receptor | 3.3 |
| 407777 | AA161071 | Hs.71465 | squalene epoxidase | 3.3 |
| 426516 | BE262660 | Hs.170197 | glutamic-oxaloacetic transaminase 2, mit | 3.3 |
| 414361 | AI086138 | Hs.204044 | ESTs | 3.3 |
| 427080 | AW068287 | Hs.173466 | ras-related C3 botulinum toxin substrate | 3.3 |
| 426429 | X73114 | Hs.169849 | myosin-binding protein C, slow-type | 3.3 |
| 446163 | AA026880 | Hs.25252 | prolactin receptor | 3.3 |
| 428566 | U41763 | Hs.184916 | clathrin, heavy polypeptide-like 1 | 3.3 |
| 418641 | BE243136 | Hs.86947 | a disintegrin and metalloproteinase doma | 3.3 |
| 436293 | AI601188 | Hs.120910 | ESTs | 3.3 |
| 411257 | AA628967 | Hs.115274 | ESTs, Highly similar to IHH_HUMAN INDIAN | 3.3 |
| 430253 | AK001514 | Hs.236844 | hypothetical protein FLJ10652 | 3.3 |
| 430066 | AI929659 | Hs.237825 | signal recognition particle 72 kD | 3.3 |
| 436469 | AK001455 | Hs.5198 | Down syndrome critical region gene 2 | 3.3 |
| 437786 | BE142681 | Hs.155573 | polymerase (DNA directed), eta | 3.3 |
| 444079 | H09048 | Hs.23606 | ESTs | 3.3 |
| 457183 | H91882 | Hs.118569 | Dvl-binding protein IDAX (inhibition of | 3.3 |
| 431215 | AA496078 | Hs.121554 | Human DNA sequence from clone RP11-218C1 | 3.3 |
| 424563 | AA446932 | Hs.151428 | ret finger protein 2 | 3.3 |
| 450828 | AW270655 | Hs.193804 | ESTs | 3.3 |
| 408652 | R43409 | Hs.6829 | Homo sapiens mRNA for KIAA1644 protein, | 3.3 |
| 445142 | AW978484 | Hs.93842 | Homo sapiens cDNA: FLJ22554 fis, clone H | 3.3 |
| 426761 | AI015709 | Hs.172089 | Homo sapiens mRNA; cDNA DKFZp586I2022 (f | 3.3 |
| 439237 | AW408158 | Hs.318893 | ESTs, Weakly similar to A47582 B-cell gr | 3.3 |
| 422616 | BE300330 | Hs.118725 | selenophosphate synthetase 2 | 3.3 |
| 443247 | BE614387 | Hs.333893 | c-Myc target JPO1 | 3.3 |
| 406663 | U24683 | Hs.302063 | immunoglobulin heavy constant mu | 3.3 |
| 434137 | AA907734 | Hs.124895 | ESTs | 3.3 |
| 408877 | AA479033 | Hs.130315 | ESTs, Weakly similar to A47582 B-cell gr | 3.3 |
| 439101 | C01765 | Hs.38750 | hypothetical protein FLJ11526 | 3.3 |
| 408221 | AA912183 | Hs.47447 | ESTs | 3.3 |
| 447519 | U46258 | Hs.339665 | ESTs | 3.3 |
| 404755 | NA | | Target Exon | 3.3 |
| 451871 | AI821005 | Hs.118599 | ESTs | 3.2 |
| 420319 | AW406289 | Hs.96593 | hypothetical protein | 3.2 |
| 430580 | AA806105 | Hs.300697 | immunoglobulin heavy constant gamma 3 (G | 3.2 |
| 400202 | NA | | NM_002795*: Homo sapiens proteasome (pros | 3.2 |
| 400222 | NA | | NM_002082*: Homo sapiens G protein-couple | 3.2 |
| 425988 | BE045897 | Hs.274454 | ESTs, Weakly similar to I38022 hypotheti | 3.2 |
| 458098 | BE550224 | Hs.74170 | metallothionein 1E (functional) | 3.2 |
| 430589 | AJ002744 | Hs.246315 | UDP-N-acetyl-alpha-D-galactosamine: polyp | 3.2 |
| 431563 | AI027643 | Hs.120912 | ESTs | 3.2 |
| 442353 | BE379594 | Hs.49136 | ESTs, Moderately similar to ALU7_HUMAN A | 3.2 |
| 422309 | U79745 | Hs.114924 | solute carrier family 16 (monocarboxylic | 3.2 |
| 419703 | AI793257 | Hs.128151 | ESTs | 3.2 |
| 420380 | AA640891 | Hs.102406 | ESTs | 3.2 |
| 410853 | H04588 | Hs.30469 | ESTs | 3.2 |
| 454417 | AI244459 | Hs.110826 | trinucleotide repeat containing 9 | 3.2 |
| 432745 | AI821926 | | gb: nt78f05.x5 NCI_CGAP_Pr3 Homo sapiens | 3.2 |
| 422032 | AA476966 | Hs.110857 | polymerase (RNA) III (DNA directed) poly | 3.2 |
| 415339 | NM_015156 | Hs.78398 | KIAA0071 protein | 3.2 |
| 426384 | AI472078 | Hs.303662 | ESTs | 3.2 |
| 448030 | N30714 | Hs.325960 | membrane-spanning 4-domains, subfamily A | 3.2 |
| 418739 | AA310964 | Hs.88012 | SHP2 interacting transmembrane adaptor | 3.2 |
| 442053 | R35343 | Hs.24968 | Human DNA sequence from clone RP1-233G16 | 3.2 |
| 434747 | AA837085 | Hs.220585 | ESTs | 3.2 |
| 427297 | AW292593 | Hs.334907 | Homo sapiens, clone MGC: 17333, mRNA, com | 3.2 |
| 412228 | AW503785 | Hs.73792 | complement component (3d/Epstein Barr vi | 3.2 |
| 452304 | AA025386 | Hs.61311 | ESTs, Weakly similar to S10590 cysteine | 3.2 |
| 453953 | AW408337 | Hs.36972 | CD7 antigen (p41) | 3.2 |
| 407758 | D50915 | Hs.38365 | KIAA0125 gene product | 3.2 |
| 451149 | AL047586 | Hs.10283 | RNA binding motif protein 8B | 3.2 |
| 430015 | AW768399 | Hs.112157 | ESTs | 3.2 |
| 433313 | W20128 | Hs.296039 | ESTs | 3.2 |
| 418334 | AA319233 | Hs.5521 | ESTs | 3.2 |
| 450223 | AA418204 | Hs.241493 | natural killer-tumor recognition sequenc | 3.2 |
| 454365 | AW966728 | Hs.54642 | methionine adenosyltransferase II, beta | 3.2 |
| 451128 | AL118668 | | gb: DKFZp761I0310_r1 761 (synonym: hamy2) | 3.2 |
| 417793 | AW405434 | Hs.82575 | small nuclear ribonucleoprotein polypept | 3.2 |
| 428027 | U22029 | Hs.334345 | cytochrome P450, subfamily IIA (phenobar | 3.2 |
| 441197 | BE244638 | Hs.166 | sterol regulatory element binding transc | 3.2 |
| 424634 | NM_003613 | Hs.151407 | cartilage intermediate layer protein, nu | 3.2 |
| 419986 | AI345455 | Hs.78915 | GA-binding protein transcription factor, | 3.2 |
| 416714 | AF283770 | Hs.79630 | CD79A antigen (immunoglobulin-associated | 3.2 |
| 449465 | NM_004380 | Hs.23598 | CREB binding protein (Rubinstein-Taybi s | 3.2 |
| 422166 | W72424 | Hs.112405 | S100 calcium-binding protein A9 (calgran | 3.2 |
| 409079 | W87707 | Hs.82065 | interleukin 6 signal transducer (gp130, | 3.2 |
| 423551 | AA327598 | Hs.233785 | ESTs | 3.2 |
| 453553 | AA036849 | Hs.61829 | Homo sapiens cDNA FLJ12763 fis, clone NT | 3.2 |
| 442580 | AI733682 | Hs.130239 | ESTs | 3.2 |
| 458079 | AI796870 | Hs.54277 | DNA segment on chromosome X (unique) 992 | 3.2 |
| 425700 | AF076292 | Hs.159251 | forkhead box H1 | 3.2 |
| 417124 | BE122762 | Hs.25338 | ESTs | 3.2 |
| 407104 | S57296 | Hs.323910 | v-erb-b2 avian erythroblastic leukemia v | 3.2 |
| 442215 | AI703172 | Hs.129005 | ESTs, Weakly similar to 2109260A B cell | 3.1 |
| 430271 | T06199 | Hs.237506 | DnaJ (Hsp40) homolog, subfamily B, membe | 3.1 |
| 425317 | AW205118 | Hs.210546 | interleukin 21 receptor | 3.1 |
| 426095 | AI278023 | Hs.89986 | ESTs | 3.1 |
| 442313 | BE388898 | Hs.8215 | hypothetical protein FLJ11307 | 3.1 |
| 424709 | AL137589 | Hs.152149 | hypothetical protein DKFZp434K0410 | 3.1 |
| 429671 | BE379335 | Hs.211594 | proteasome (prosome, macropain) 26S subu | 3.1 |
| 432715 | AA247152 | Hs.200483 | ESTs, Weakly similar to KIAA1074 protein | 3.1 |
| 431574 | AW572659 | Hs.261373 | hypothetical protein dJ434O14.3 | 3.1 |
| 436876 | AI124756 | Hs.5337 | isocitrate dehydrogenase 2 (NADP), mitoc | 3.1 |
| 405017 | NA | | Target Exon | 3.1 |
| 433805 | AA706910 | Hs.112742 | ESTs | 3.1 |
| 437352 | AL353957 | Hs.284181 | hypothetical protein DKFZp434P0531 | 3.1 |
| 430105 | X70297 | Hs.2540 | cholinergic receptor, nicotinic, alpha p | 3.1 |
| 422083 | NM_001141 | Hs.111256 | arachidonate 15-lipoxygenase, second typ | 3.1 |
| 413507 | BE145360 | Hs.190064 | ESTs, Weakly similar to I38022 hypotheti | 3.1 |
| 415989 | AI267700 | Hs.317584 | ESTs | 3.1 |
| 422907 | AI879263 | Hs.6986 | Human glucose transporter pseudogene | 3.1 |
| 425548 | AA890023 | Hs.1906 | prolactin receptor | 3.1 |
| 422599 | BE387202 | Hs.118638 | non-metastatic cells 1, protein (NM23A) | 3.1 |
| 439963 | AW247529 | Hs.6793 | platelet-activating factor acetylhydrola | 3.1 |
| 453883 | AI638516 | Hs.22630 | cofactor required for Sp1 transcriptiona | 3.1 |
| 458021 | AI885190 | Hs.156089 | ESTs, Weakly similar to repressor protei | 3.1 |
| 418478 | U38945 | Hs.1174 | cyclin-dependent kinase inhibitor 2A (me | 3.1 |
| 400814 | NA | | Target Exon | 3.1 |
| 402327 | | | Target Exon | 3.1 |
| 416935 | AA190712 | | gb: zp87f09.r1 Stratagene HeLa cell s3 93 | 3.1 |
| 439838 | AL355722 | Hs.106875 | Homo sapiens EST from clone 35214, full | 3.1 |
| 437036 | AI571514 | Hs.133022 | ESTs | 3.1 |
| 449523 | NM_000579 | Hs.54443 | chemokine (C-C motif) receptor 5 | 3.1 |
| 406642 | AJ245210 | | gb: Homo sapiens mRNA for immunoglobulin | 3.1 |
| 406624 | AF052762 | | gb: Homo sapiens clone csneg8-1 immunoglo | 3.1 |
| 421924 | BE514514 | Hs.109606 | coronin, actin-binding protein, 1A | 3.1 |
| 414523 | AU076633 | Hs.76353 | serine (or cysteine) proteinase inhibito | 3.1 |
| 416379 | N38857 | Hs.203933 | ESTs | 3.1 |
| 422823 | D89974 | Hs.121102 | vanin 2 | 3.1 |
| 433904 | AI399956 | Hs.208956 | ESTs | 3.1 |
| 421904 | BE143533 | Hs.109309 | hypothetical protein FLJ20035 | 3.1 |
| 428834 | AW899713 | Hs.339315 | ESTs | 3.1 |
| 436043 | AW963838 | Hs.168830 | Homo sapiens cDNA FLJ12136 fis, clone MA | 3.1 |
| 452823 | AB012124 | Hs.30696 | transcription factor-like 5 (basic helix | 3.1 |
| 405381 | NA | | Target Exon | 3.1 |
| 428746 | AW503820 | Hs.192861 | Spi-B transcription factor (Spi-1/PU.1 r | 3.1 |
| 435147 | AL133731 | Hs.4774 | Homo sapiens mRNA; cDNA DKFZp761C1712 (f | 3.1 |
| 425782 | U66468 | Hs.159525 | cell growth regulatory with EF-hand doma | 3.1 |
| 423306 | W88562 | Hs.108198 | ESTs | 3.1 |
| 419123 | AA234276 | Hs.88253 | ESTs | 3.1 |
| 438581 | AW977766 | Hs.292133 | ESTs, Moderately similar to I78885 serin | 3.1 |
| 417105 | X60992 | Hs.81226 | CD6 antigen | 3.0 |
| 428361 | NM_015905 | Hs.183858 | transcriptional intermediary factor 1 | 3.0 |
| 417880 | BE241595 | Hs.82848 | selectin L (lymphocyte adhesion molecule | 3.0 |
| 402606 | | | NM_024626: Homo sapiens hypothetical prot | 3.0 |
| 401451 | | | NM_004496*: Homo sapiens hepatocyte nucle | 3.0 |
| 421878 | AA299652 | Hs.111496 | Homo sapiens cDNA FLJ11643 fis, clone HE | 3.0 |
| 409518 | BE384836 | Hs.3454 | KIAA1821 protein | 3.0 |
| 416933 | BE561850 | Hs.80506 | small nuclear ribonucleoprotein polypept | 3.0 |
| 414324 | Y14768 | Hs.890 | lymphotoxin beta (TNF superfamily, membe | 3.0 |
| 425081 | X74794 | Hs.154443 | minichromosome maintenance deficient (S. | 3.0 |
| 401519 | | | C15000476*: gi\|12737279\|ref\|XP_012163.1\| | 3.0 |
| 411704 | AI499220 | Hs.71573 | hypothetical protein FLJ10074 | 3.0 |
| 428819 | AL135623 | Hs.193914 | KIAA0575 gene product | 3.0 |
| 428423 | AU076517 | Hs.184276 | solute carrier family 9 (sodium/hydrogen | 3.0 |
| 413835 | AI272727 | Hs.249163 | fatty acid hydroxylase | 3.0 |
| 412600 | L28824 | Hs.74101 | spleen tyrosine kinase | 3.0 |
| 410491 | AA465131 | Hs.64001 | Homo sapiens clone 25218 mRNA sequence | 3.0 |
| 433658 | L03678 | Hs.156110 | immunoglobulin kappa constant | 3.0 |
| 427666 | AI791495 | Hs.180142 | calmodulin-like skin protein | 3.0 |
| 452514 | AI904898 | | gb: RC-BT068-130399-085 BT068 Homo sapien | 3.0 |
| 429500 | X78565 | Hs.289114 | hexabrachion (tenascin C, cytotactin) | 3.0 |
| 432485 | N90866 | Hs.276770 | CDW52 antigen (CAMPATH-1 antigen) | 3.0 |
| 437400 | AB011542 | Hs.5599 | EGE-like-domain, multiple 5 | 3.0 |
| 452234 | AW084176 | Hs.223296 | ESTs, Weakly similar to I38022 hypotheti | 3.0 |
| 413269 | BE167526 | | gb: CM4-HT0509-080300-107-g07 HT0509 Homo | 3.0 |
| 453216 | AL137566 | Hs.32405 | Homo sapiens mRNA; cDNA DKFZp586G0321 (f | 3.0 |
| 400929 | | | ENSP00000252232*: Sterol regulatory eleme | 3.0 |
| 445145 | AI961702 | Hs.147434 | ESTs | 3.0 |
| 432615 | AA557191 | Hs.55028 | ESTs, Weakly similar to I54374 gene NF2 | 3.0 |
| 423279 | AW959861 | Hs.290943 | ESTs | 3.0 |
| 429392 | AL109712 | Hs.296506 | Homo sapiens mRNA full length insert cDN | 3.0 |
| 408548 | AA055449 | Hs.63187 | ESTs, Weakly similar to ALUC_HUMAN !!!! | 3.0 |
| 451346 | NM_006338 | Hs.26312 | glioma amplified on chromosome 1 protein | 3.0 |
| 413109 | AW389845 | Hs.110855 | ESTs | 3.0 |
| 401714 | NA | | ENSP00000241802*: CDNA FLJ11007 FIS, CLON | 3.0 |
| 421462 | AF016495 | Hs.104624 | aquaporin 9 | 3.0 |
| 421750 | AK000768 | Hs.107872 | hypothetical protein FLJ20761 | 3.0 |
| 453293 | AA382267 | Hs.10653 | ESTs | 3.0 |
| 457085 | AA412446 | Hs.98138 | ESTs | 3.0 |
| 438930 | AW843633 | Hs.306163 | hypothetical protein AL110115 | 3.0 |

Table 22 shows 739 genes up-regulated in breast cancer compared to normal adult breast. These were selected as for Table 19, except that the ratio was greater than or equal to 3.0, the denominator was the 85^{th} percentile value for 12 non-malignant breast specimens, and the 96^{th} percentile value amongst the 73 breast cancers was greater than or equal 100 units.
Pkey: Unique Eos probeset identifier number
ExAccn: Exemplar Accession number, Genbank accession number
UnigeneID: Unigene number
Unigene Title: Unigene gene title
R1: Ratio of 90^{th} percentile tumor to 85^{th} percentile normal breast tissue

TABLE 22A
Table 22A shows the accession numbers for those
pkeys lacking unigeneID's for Table 22.
For each probeset, we have listed the gene cluster
number from which the oligonucleotides were designed.
Gene clusters were compiled using sequences derived
from Genbank ESTs and mRNAs. These sequences were
clustered based on sequence similarity using Clustering
and Alignment Tools (DoubleTwist, Oakland California).
The Genbank accession numbers for sequences comprising
each cluster are listed in the “Accession” column.

| Pkey | CAT number | Accessions |
|---|---|---|
| 407980 | 103087_1 | AA046309 AI263500 AA046397 |
| 410785 | 1221055_1 | AW803341 AW803265 AW803403 AW803466 |
| | | AW803402 AW803413 AW803268 |
| | | AW803396 AW803334 AW803355 |
| 411743 | 1256098_1 | AW862214 AW859811 AW862215 |
| 412138 | 1279172_1 | AW895387 AW895547 AW895564 |
| | | AW895323 AW895405 AW895539 AW895538 |
| 413269 | 1356961_1 | BE167526 BE167651 BE076401 R24654 |
| 416935 | 163179_1 | AA190712 AA190665 AA252564 |
| 422128 | 211994_1 | AW881145 AA490718 M85637 AA304575 |
| | | T06067 AA331991 |
| 423945 | 233566_1 | AA410943 AW948953 AA334202 AA332882 |
| 424109 | 235506_1 | AW406878 AW966560 AW966151 AW966496 |
| | | AA336174 AA335376 AA335537 |
| 424128 | 235728_1 | AW966163 AA335983 AA336011 |
| | | AA335668 AA335973 |
| 425331 | 250199_1 | AW962128 AA355353 AA427363 |
| 426878 | 273265_1 | BE069341 AW748403 AL044891 |
| | | AI908240 AA393080 |
| 432745 | 353673_1 | AI821926 AA658826 AA564492 |
| | | AA635129 AI791191 |
| 441153 | 51084_2 | BE562826 BE378727 |
| 448212 | 755099_1 | AI475858 AW969013 |
| 451128 | 859865_1 | AL118668 D78823 AI762176 |
| 452514 | 920172_1 | AI904898 AI904849 AI904899 |
| 456207 | 165078_−1 | AA193450 |

Pkey: Unique Eos probeset identifier number
CAT number: Gene cluster numbe
Accession: Genbank accession numbers

TABLE 22B
Table 22B shows the genomic positioning
for those pkeys lacking unigene ID's and
accession numbers in Table 22. For each
predicted exon, we have listed the genomic
sequence source used for prediction.
Nucleotide locations of each predicted exon
are also listed.

| Pkey | Ref | Strand | Nt_position |
|---|---|---|---|
| 400814 | 8569925 | Minus | 72840-72924, 74761-74849 |
| 400929 | 7651921 | Minus | 122033-122241, 123483-124028 |
| 401045 | 8117619 | Plus | 90044-90184, 91111-91345 |
| 401451 | 6634068 | Minus | 119926-121272 |
| 401519 | 6649315 | Plus | 157315-157950 |
| 401645 | 7657839 | Minus | 34986-35133 |
| 401714 | 6715702 | Plus | 96484-96681 |
| 401866 | 8018106 | Plus | 73126-73623 |
| 402327 | 7656695 | Minus | 108675-108770, 109801-109910 |
| 402359 | 9211204 | Minus | 40403-41961 |
| 402408 | 9796239 | Minus | 110326-110491 |
| 402470 | 9797107 | Plus | 195129-195776 |
| 402542 | 9801558 | Minus | 67076-67594 |
| 402578 | 9884928 | Plus | 66350-66496 |
| 402606 | 9909429 | Minus | 81747-82094 |
| 403011 | 6693597 | Minus | 3468-3623 |
| 403212 | 7630897 | Minus | 156037-156210 |
| 403329 | 8516120 | Plus | 96450-96598 |
| 403366 | 8783692 | Minus | 49323-49652 |
| 403485 | 9966528 | Plus | 2888-3001, 3198-3532, 3655-4117 |
| 404347 | 9838195 | Plus | 74493-74829 |
| 404580 | 6539738 | Minus | 240588-241589 |
| 404755 | 7706327 | Minus | 53729-53846 |
| 405017 | 6532084 | Plus | 35551-35690 |
| 405348 | 2914717 | Minus | 43310-43462 |
| 405381 | 6006920 | Minus | 7636-8054 |
| 405801 | 2924321 | Plus | 63469-63694 |
| 405850 | 6164995 | Plus | 13871-14110 |
| 406153 | 9929734 | Minus | 12902-13069 |
| 406348 | 9255985 | Minus | 71754-71944 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 23
320 GENES DOWN-REGULATED IN BREAST CANCER
COMPARED TO NORMAL ADULT BREAST

| Pkey | ExAccn | UnigeneID | UnigeneTitle | Ratio |
|---|---|---|---|---|
| 428722 | U76456 | Hs.190787 | tissue inhibitor of metalloproteinase 4 | 22.4 |
| 428848 | NM_000230 | Hs.194236 | leptin (murine obesity homolog) | 17.4 |
| 445263 | H57646 | Hs.42586 | KIAA1560 protein | 15.4 |
| 418935 | T28499 | Hs.89485 | carbonic anhydrase IV | 15.0 |
| 407228 | M25079 | Hs.155376 | hemoglobin, beta | 14.6 |
| 417511 | AL049176 | Hs.82223 | chordin-like | 14.6 |
| 439285 | AL133916 | Hs.172572 | hypothetical protein FLJ20093 | 14.3 |
| 412442 | AI983730 | Hs.26530 | serum deprivation response (phosphatidyl | 13.6 |
| 410544 | AI446543 | Hs.95511 | ESTs | 12.6 |
| 412047 | AA934589 | Hs.49696 | ESTs | 12.2 |
| 422667 | H25642 | Hs.133471 | ESTs | 12.0 |
| 406664 | L34041 | Hs.9739 | glycerol-3-phosphate dehydrogenase 1 (so | 12.0 |
| 423201 | NM_000163 | Hs.125180 | growth hormone receptor | 11.7 |
| 422163 | AF027208 | Hs.112360 | prominin (mouse)-like 1 | 10.8 |
| 428769 | AW207175 | Hs.106771 | ESTs | 10.6 |
| 407049 | X72632 | | NM_021724*: Homo sapiens nuclear receptor | 10.1 |
| 412295 | AW088826 | Hs.117176 | poly(A)-binding protein, nuclear 1 | 9.8 |
| 425126 | N32759 | Hs.172944 | chorionic gonadotropin, beta polypeptide | 9.8 |
| 406791 | AI220684 | Hs.272572 | hemoglobin, alpha 2 | 9.5 |
| 447471 | AF039843 | Hs.18676 | sprouty (Drosophila) homolog 2 | 9.5 |
| 451533 | NM_004657 | Hs.26530 | serum deprivation response (phosphatidyl | 9.4 |
| 419407 | AW410377 | Hs.41502 | hypothetical protein FLJ21276 | 9.0 |
| 411939 | AI365585 | Hs.146246 | ESTs | 9.0 |
| 410532 | T53088 | Hs.155376 | hemoglobin, beta | 8.9 |
| 425707 | AF115402 | Hs.11713 | E74-like factor 5 (ets domain transcript | 8.8 |
| 416585 | X54162 | Hs.79386 | leiomodin 1 (smooth muscle) | 8.7 |
| 443060 | D78874 | Hs.8944 | procollagen C-endopeptidase enhancer 2 | 8.6 |
| 435265 | AA779958 | Hs.185932 | ESTs | 8.5 |
| 422511 | AU076442 | Hs.117938 | collagen, type XVII, alpha 1 | 8.4 |
| 433138 | AB029496 | Hs.59729 | semaphorin sem2 | 8.3 |
| 402195 | | | NM_004497*: Homo sapiens hepatocyte nucle | 8.1 |
| 429350 | AI754634 | Hs.131987 | ESTs | 8.1 |
| 445107 | AI208121 | Hs.147313 | ESTs, Weakly similar to I38022 hypotheti | 8.1 |
| 406643 | N77976 | Hs.272572 | hemoglobin, alpha 2 | 8.0 |
| 410199 | AW377424 | Hs.205126 | Homo sapiens cDNA: FLJ22667 fis, clone H | 8.0 |
| 417225 | AA815048 | Hs.24078 | hypothetical protein FLJ12649 | 7.8 |
| 437569 | AA760849 | Hs.294052 | ESTs | 7.5 |
| 436062 | AK000027 | Hs.98633 | ESTs | 7.5 |
| 425078 | NM_002599 | Hs.154437 | phosphodiesterase 2A, cGMP-stimulated | 7.5 |
| 430327 | AW973636 | Hs.55931 | ESTs | 7.4 |
| 447577 | AI393693 | Hs.183297 | DKFZP566F2124 protein | 7.4 |
| 446039 | AI150491 | Hs.90756 | ESTs | 7.2 |
| 422060 | R20893 | Hs.325823 | ESTs, Moderately similar to ALU5_HUMAN A | 7.2 |
| 424455 | AA452006 | Hs.333199 | ESTs | 7.1 |
| 424343 | AW956360 | Hs.4748 | adenylate cyclase activating polypeptide | 7.1 |
| 442792 | AI352340 | Hs.131194 | ESTs | 7.0 |
| 406714 | AI219304 | Hs.283108 | hemoglobin, gamma G | 6.9 |
| 407571 | AI446183 | Hs.9572 | ESTs, Highly similar to CYA5_HUMAN ADENY | 6.8 |
| 429580 | AA346839 | Hs.209100 | DKFZP434C171 protein | 6.7 |
| 453500 | AI478427 | Hs.43125 | esophageal cancer related gene 4 protein | 6.7 |
| 422233 | AB002058 | Hs.113275 | purinergic receptor P2X-like 1, orphan r | 6.7 |
| 420205 | AA256395 | Hs.88156 | ESTs | 6.6 |
| 404368 | NA | | ENSP00000241075*: TRRAP PROTEIN. | 6.6 |
| 447261 | NM_006691 | Hs.17917 | extracellular link domain-containing 1 | 6.5 |
| 417090 | AA193282 | Hs.85863 | ESTs, Weakly similar to B34612 zinc fing | 6.5 |
| 410677 | NM_003278 | Hs.65424 | tetranectin (plasminogen-binding protein | 6.5 |
| 427283 | AL119796 | Hs.174185 | ectonucleotide pyrophosphatase/phosphodi | 6.5 |
| 415011 | AW963085 | | gb: EST375158 MAGE resequences, MAGH Homo | 6.4 |
| 412068 | S72043 | Hs.73133 | metallothionein 3 (growth inhibitory fac | 6.4 |
| 416253 | BE250659 | Hs.15463 | Homo sapiens, clone IMAGE: 2959994, mRNA | 6.4 |
| 435885 | AA701483 | Hs.36341 | ESTs | 6.3 |
| 402779 | NA | | Target Exon | 6.3 |
| 418138 | AA213626 | Hs.136204 | EST | 6.3 |
| 439335 | AA742697 | Hs.62492 | ESTs, Weakly similar to B39066 proline-r | 6.3 |
| 427019 | AA001732 | Hs.173233 | hypothetical protein FLJ10970 | 6.2 |
| 411478 | BE143068 | | gb: MR0-HT0158-030200-003-b09 HT0158 Homo | 6.2 |
| 452654 | BE004783 | | gb: MR2-BN0114-270400-004-e11 BN0114 Homo | 6.1 |
| 447359 | NM_012093 | Hs.18268 | adenylate kinase 5 | 6.1 |
| 414323 | NM_014759 | Hs.334688 | KIAA0273 gene product | 6.1 |
| 441266 | H15968 | Hs.293845 | Homo sapiens, clone IMAGE: 3502329, mRNA, | 6.1 |
| 417011 | F08212 | Hs.234898 | ESTs, Weakly similar to 2109260A B cell | 6.0 |
| 400089 | NA | | Eos Control | 6.0 |
| 433614 | W07475 | Hs.277101 | cytochrome c oxidase subunit IV isoform | 5.9 |
| 440439 | N92818 | Hs.64754 | ESTs, Weakly similar to potential CDS [H | 5.9 |
| 454404 | BE067414 | | gb: MR4-BT0355-200100-201-e05 BT0355 Homo | 5.9 |
| 436704 | AA062610 | Hs.148050 | EST | 5.9 |
| 406563 | NA | | Target Exon | 5.9 |
| 433490 | AW451023 | Hs.65848 | hypothetical protein DKFZp761O132 | 5.9 |
| 419313 | AA843387 | Hs.87279 | ESTs | 5.9 |
| 409196 | NM_001874 | Hs.334873 | carboxypeptidase M | 5.8 |
| 410882 | AW809163 | | gb: MR4-ST0118-261099-012-a03 ST0118 Homo | 5.8 |
| 453469 | AB014533 | Hs.33010 | KIAA0633 protein | 5.8 |
| 441899 | AI372588 | Hs.8022 | TU3A protein | 5.8 |
| 426210 | AA372052 | Hs.334559 | Homo sapiens cDNA FLJ14458 fis, clone HE | 5.8 |
| 413065 | BE063555 | | gb: CM1-BT0283-081199-033-d09 BT0283 Homo | 5.8 |
| 454192 | AW876813 | Hs.3343 | phosphoglycerate dehydrogenase | 5.7 |
| 425187 | AW014486 | Hs.22509 | ESTs | 5.7 |
| 429757 | AW452355 | Hs.256037 | ESTs | 5.7 |
| 420202 | AL036557 | Hs.95910 | putative lymphocyte G0/G1 switch gene | 5.7 |
| 416284 | AI695473 | Hs.298006 | ESTs | 5.7 |
| 428553 | AA181641 | Hs.184907 | G protein-coupled receptor 1 | 5.6 |
| 404689 | NA | | Target Exon | 5.6 |
| 438887 | R68857 | Hs.265499 | ESTs | 5.6 |
| 406082 | S47833 | Hs.82927 | adenosine monophosphate deaminase 2 (iso | 5.6 |
| 449748 | H23963 | Hs.32043 | ESTs | 5.6 |
| 431048 | R50253 | Hs.249129 | cell death-inducing DFFA-like effector a | 5.5 |
| 452205 | C15819 | | gb: C15819 Clontech human aorta polyA mRN | 5.5 |
| 434040 | AW444613 | Hs.288809 | hypothetical protein FLJ20159 | 5.5 |
| 407744 | AB020629 | Hs.38095 | ATP-binding cassette, sub-family A (ABC 1 | 5.5 |
| 450606 | AI668605 | Hs.60380 | ESTs, Moderately similar to ALU6_HUMAN A | 5.5 |
| 414629 | AA345824 | Hs.76688 | carboxylesterase 1 (monocyte/macrophage | 5.5 |
| 401665 | | | C11000703: gi\|10048448\|ref\|NP_065258.1\|g | 5.5 |
| 436107 | T99079 | Hs.191194 | ESTs | 5.5 |
| 444432 | AI161428 | Hs.75916 | splicing factor 3b, subunit 2, 145 kD | 5.5 |
| 434715 | BE005346 | Hs.116410 | ESTs | 5.5 |
| 447205 | BE617015 | Hs.11006 | ESTs, Moderately similar to T17372 plasm | 5.5 |
| 408122 | AI432652 | Hs.42824 | hypothetical protein FLJ10718 | 5.5 |
| 454016 | AW016806 | Hs.233108 | ESTs | 5.5 |
| 414913 | R25621 | | gb: yh45f06.r1 Soares placenta Nb2HP Homo | 5.4 |
| 459033 | AA017590 | Hs.129907 | ESTs | 5.4 |
| 441003 | BE172240 | Hs.126379 | ESTs, Weakly similar to I38022 hypotheti | 5.4 |
| 450637 | N49826 | Hs.18602 | ESTs | 5.4 |
| 442398 | AA994520 | | gb: ou42g09.s1 Soares_NFL_T_GBC_S1 Homo s | 5.4 |
| 403612 | NA | | Target Exon | 5.3 |
| 407102 | AA007629 | Hs.9739 | glycerol-3-phosphate dehydrogenase 1 (so | 5.3 |
| 410057 | R66634 | Hs.268107 | multimerin | 5.3 |
| 428232 | BE272452 | Hs.183109 | monoamine oxidase A | 5.3 |
| 432769 | AA620814 | Hs.144959 | ESTs | 5.3 |
| 431344 | R99530 | Hs.272572 | hemoglobin, alpha 2 | 5.3 |
| 427032 | AF012023 | Hs.173274 | integrin cytoplasmic domain-associated p | 5.3 |
| 406305 | BE261320 | Hs.158196 | transcriptional adaptor 3 (ADA3, yeast h | 5.3 |
| 437411 | AW613948 | Hs.194915 | ESTs | 5.3 |
| 442800 | AI809481 | Hs.131227 | ESTs | 5.3 |
| 402054 | NA | | Target Exon | 5.3 |
| 432085 | AF212829 | Hs.272406 | potassium channel, subfamily K, member 9 | 5.3 |
| 415313 | R59638 | Hs.6181 | ESTs | 5.2 |
| 459159 | AI904646 | | gb: QV-BT065-020399-103 BT065 Homo sapien | 5.2 |
| 427164 | AB037721 | Hs.173871 | KIAA1300 protein | 5.2 |
| 441391 | BE467930 | Hs.170381 | ESTs | 5.2 |
| 458959 | AI285901 | Hs.181297 | ESTs | 5.2 |
| 402698 | NA | | ENSP00000251335*: DJ1003J2.1 (sodium and | 5.2 |
| 401810 | NA | | Target Exon | 5.2 |
| 438879 | AA827674 | Hs.189073 | ESTs | 5.2 |
| 414657 | AA424074 | Hs.76780 | protein phosphatase 1, regulatory (inhib | 5.2 |
| 427809 | M26380 | Hs.180878 | lipoprotein lipase | 5.1 |
| 456063 | NM_006744 | Hs.76461 | retinol-binding protein 4, interstitial | 5.1 |
| 451186 | AW023469 | Hs.65256 | ESTs, Weakly similar to leucine-rich gli | 5.1 |
| 451882 | AI821324 | Hs.100445 | ESTs | 5.1 |
| 402583 | NA | | NM_021620: Homo sapiens PR domain contain | 5.1 |
| 431130 | NM_006103 | Hs.2719 | HE4; WFDC2; putative ovarian carcinoma m | 5.1 |
| 458218 | AI435179 | Hs.126820 | ESTs | 5.1 |
| 416083 | R53467 | Hs.269122 | ESTs, Weakly similar to ALU1_HUMAN ALU S | 5.1 |
| 455282 | BE143867 | | gb: MR0-HT0164-070100-013-h02 HT0164 Homo | 5.1 |
| 426488 | X03350 | Hs.4 | alcohol dehydrogenase 1B (class I), beta | 5.1 |
| 426156 | BE244537 | Hs.167382 | natriuretic peptide receptor A/guanylate | 5.1 |
| 407891 | AA486620 | Hs.41135 | endomucin-2 | 5.0 |
| 408610 | AW026692 | Hs.224829 | ESTs | 5.0 |
| 445967 | D59597 | Hs.118821 | CGI-62 protein | 5.0 |
| 434813 | AI524307 | Hs.162870 | ESTs | 5.0 |
| 437526 | AI076012 | Hs.121388 | ESTs, Weakly similar to MDHC_HUMAN MALAT | 5.0 |
| 454775 | BE160229 | | gb: QV1-HT0413-090200-062-a12 HT0413 Homo | 5.0 |
| 409451 | AF012626 | Hs.54472 | fragile X mental retardation 2 | 5.0 |
| 409853 | AW502327 | | gb: UI-HF-BR0p-aka-a-07-0-UI.r1 NIH_MGC_5 | 5.0 |
| 405062 | | | Target Exon | 5.0 |
| 446490 | AK000706 | Hs.15125 | hypothetical protein FLJ20699 | 5.0 |
| 417622 | AW298163 | Hs.82318 | WAS protein family, member 3 | 5.0 |
| 421978 | AJ243662 | Hs.110196 | NICE-1 protein | 5.0 |
| 440338 | R62431 | Hs.12758 | ESTs | 5.0 |
| 415421 | R35009 | Hs.24903 | ESTs | 5.0 |
| 417574 | R00348 | | gb: ye69e06.r1 Soares fetal liver spleen | 5.0 |
| 409882 | AJ243191 | Hs.56874 | heat shock 27 kD protein family, member 7 | 5.0 |
| 447998 | AI768289 | Hs.304389 | ESTs | 4.9 |
| 445613 | BE550889 | Hs.158491 | ESTs | 4.9 |
| 443074 | AW341470 | Hs.144907 | ESTs | 4.9 |
| 451324 | AI783600 | Hs.208052 | ESTs | 4.9 |
| 432433 | AW014734 | Hs.157969 | ESTs | 4.9 |
| 449654 | AI989812 | Hs.199850 | ESTs | 4.9 |
| 414519 | N94587 | Hs.55063 | ESTs | 4.9 |
| 457531 | AW973716 | Hs.13913 | KIAA1577 protein | 4.9 |
| 433200 | AA682722 | Hs.192725 | ESTs | 4.9 |
| 430782 | AF026263 | Hs.247920 | cholinergic receptor, muscarinic 5 | 4.8 |
| 427555 | AW137094 | Hs.97990 | ESTs | 4.8 |
| 433545 | AA868510 | Hs.112496 | ESTs | 4.8 |
| 420334 | AI349351 | Hs.118944 | hypothetical protein FLJ22477 | 4.8 |
| 421795 | X63094 | Hs.283822 | Rhesus blood group, D antigen | 4.8 |
| 427138 | N77624 | Hs.173717 | phosphatidic acid phosphatase type 2B | 4.8 |
| 413072 | BE063965 | | gb: QV3-BT0296-140200-085-h01 BT0296 Homo | 4.8 |
| 443721 | AW450451 | Hs.266355 | ESTs | 4.8 |
| 408053 | AW139474 | Hs.246862 | ESTs | 4.8 |
| 427067 | AA843716 | Hs.177927 | ESTs | 4.7 |
| 442969 | AI025499 | Hs.132238 | ESTs | 4.7 |
| 426220 | AI383475 | Hs.171697 | ESTs, Weakly similar to T13924 sdk prote | 4.7 |
| 414593 | BE386764 | | gb: 601273249F1 NIH_MGC_20 Homo sapiens c | 4.7 |
| 426893 | AA398716 | Hs.97418 | ESTs | 4.7 |
| 434046 | AW292618 | Hs.113011 | ESTs | 4.7 |
| 401590 | NA | | Target Exon | 4.7 |
| 457971 | AW134679 | Hs.242849 | ESTs | 4.7 |
| 427722 | AK000123 | Hs.180479 | hypothetical protein FLJ20116 | 4.6 |
| 443793 | AA045290 | Hs.25930 | ESTs, Weakly similar to 2109260A B cell | 4.6 |
| 407737 | R49187 | Hs.6659 | ESTs | 4.6 |
| 441955 | AA972327 | Hs.142903 | ESTs | 4.6 |
| 441499 | AW298235 | Hs.101689 | ESTs | 4.6 |
| 447517 | AI382726 | Hs.182434 | ESTs | 4.6 |
| 403017 | | | Target Exon | 4.6 |
| 450580 | N40087 | Hs.15248 | ESTs | 4.6 |
| 404611 | H58589 | Hs.35156 | Homo sapiens cDNA FLJ11027 fis, clone PL | 4.6 |
| 414831 | M31158 | Hs.77439 | protein kinase, cAMP-dependent, regulato | 4.6 |
| 459290 | NM_001546 | Hs.34853 | inhibitor of DNA binding 4, dominant neg | 4.6 |
| 444341 | AI142027 | Hs.146650 | ESTs | 4.6 |
| 408614 | AL137698 | Hs.46531 | Homo sapiens mRNA; cDNA DKFZp434C1915 (f | 4.6 |
| 449638 | AW204277 | Hs.250723 | hypothetical protein MGC2747 | 4.6 |
| 434418 | AF134707 | Hs.278679 | a disintegrin and metalloproteinase doma | 4.6 |
| 447360 | AI375984 | Hs.167216 | ESTs | 4.6 |
| 419583 | F00312 | | gb: HSBB0D101 STRATAGENE Human skeletal m | 4.6 |
| 440698 | AI348455 | Hs.147492 | Homo sapiens cDNA FLJ11777 fis, clone HE | 4.6 |
| 451199 | AI290653 | Hs.124758 | ESTs | 4.6 |
| 438338 | NM_014861 | Hs.6168 | KIAA0703 gene product | 4.6 |
| 433756 | AW015933 | Hs.112654 | Homo sapiens, clone MGC: 9764, mRNA, comp | 4.5 |
| 423301 | S67580 | Hs.1645 | cytochrome P450, subfamily IVA, polypept | 4.5 |
| 417237 | H86385 | Hs.81737 | palmitoyl-protein thioesterase 2 | 4.5 |
| 439745 | AL389981 | Hs.149219 | Homo sapiens mRNA full length insert cDN | 4.5 |
| 424137 | AA335769 | Hs.16262 | ESTs | 4.5 |
| 449338 | H73444 | Hs.394 | adrenomedullin | 4.5 |
| 434744 | N94835 | Hs.283828 | Homo sapiens genomic DNA, chromosome 21q | 4.5 |
| 407402 | AF035303 | | gb: Homo sapiens clone 23943 mRNA sequenc | 4.5 |
| 443510 | NM_012190 | Hs.9520 | formyltetrahydrofolate dehydrogenase | 4.5 |
| 415754 | AA169114 | Hs.12247 | hypothetical protein FLJ11413 | 4.5 |
| 415986 | Z43619 | | gb: HSC1GE121 normalized infant brain cDN | 4.5 |
| 457416 | BE142052 | Hs.62654 | kringle-containing transmembrane protein | 4.5 |
| 418064 | BE387287 | Hs.83384 | S100 calcium-binding protein, beta (neur | 4.4 |
| 437120 | AI356125 | Hs.157767 | ESTs, Weakly similar to HXA2_HUMAN HOMEO | 4.4 |
| 453950 | AA156998 | Hs.211568 | eukaryotic translation initiation factor | 4.4 |
| 401093 | | | C12000586*: gi\|6330167\|dbj\|BAA86477.1\| (A | 4.4 |
| 436935 | AW206494 | Hs.253560 | ESTs | 4.4 |
| 457974 | AW842353 | Hs.321717 | ESTs, Weakly similar to S22765 heterogen | 4.4 |
| 428222 | AL133112 | Hs.183085 | Homo sapiens mRNA; cDNA DKFZp434K098 (fr | 4.4 |
| 442705 | AI264634 | Hs.131127 | ESTs | 4.4 |
| 437409 | AL359599 | Hs.283850 | Homo sapiens mRNA; cDNA DKFZp547C126 (fr | 4.4 |
| 458494 | AI380906 | Hs.158436 | ESTs | 4.4 |
| 410490 | H03589 | | gb: yj42d08.r1 Soares placenta Nb2HP Homo | 4.4 |
| 416069 | R37101 | Hs.20982 | ESTs | 4.4 |
| 438463 | AA807958 | Hs.314232 | ESTs | 4.4 |
| 444043 | AI499723 | Hs.135089 | ESTs | 4.4 |
| 438327 | H87407 | Hs.172944 | chorionic gonadotropin, beta polypeptide | 4.4 |
| 457711 | AF147401 | Hs.23917 | ESTs | 4.3 |
| 400870 | | | C11000905: gi\|11692565\|gb\|AAG39879.1\|AF28 | 4.3 |
| 441425 | AA933590 | Hs.28937 | homeobox protein from AL590526 | 4.3 |
| 416267 | H45384 | | gb: yn99c10.r1 Soares adult brain N2b5HB5 | 4.3 |
| 407262 | M12873 | | gb: Human Ig rearranged H-chain mRNA VDJ4 | 4.3 |
| 444567 | AV654020 | Hs.184261 | ESTs, Weakly similar to T26686 hypotheti | 4.3 |
| 403263 | NA | | Target Exon | 4.3 |
| 410034 | BE067414 | | gb: MR4-BT0355-200100-201-e05 BT0355 Homo | 4.3 |
| 456804 | AI421645 | Hs.139851 | caveolin 2 | 4.3 |
| 448427 | BE395260 | Hs.309438 | EST | 4.3 |
| 416931 | D45371 | Hs.80485 | adipose most abundant gene transcript 1 | 4.3 |
| 421296 | NM_002666 | Hs.103253 | perilipin | 4.3 |
| 400973 | NA | | ENSP00000236667*: Mucin 5B (Fragment). | 4.3 |
| 452602 | AW366194 | Hs.55962 | ESTs | 4.3 |
| 412330 | NM_005100 | Hs.788 | A kinase (PRKA) anchor protein (gravin) | 4.3 |
| 405016 | | | CY000171*: gi\|9280405\|gb\|AAF86402.1\|AF245 | 4.3 |
| 435104 | AI475671 | Hs.88607 | ESTs, Highly similar to F-box protein FB | 4.3 |
| 406118 | | | ENSP00000246632: CDNA FLJ20261 fis, clone | 4.3 |
| 418556 | T02850 | | gb: FB12A9 Fetal brain, Stratagene Homo s | 4.3 |
| 429745 | AA480818 | Hs.221736 | ESTs | 4.3 |
| 433088 | AW451206 | Hs.115899 | ESTs | 4.3 |
| 444445 | AA342329 | Hs.115920 | Homo sapiens cDNA: FLJ22816 fis, clone K | 4.3 |
| 453880 | AI803166 | Hs.28462 | ESTs, Weakly similar to I38022 hypotheti | 4.3 |
| 447384 | AI377221 | Hs.40528 | ESTs | 4.2 |
| 414541 | BE293116 | Hs.76392 | aldehyde dehydrogenase 1 family, member | 4.2 |
| 444975 | AV652165 | Hs.182482 | ESTs, Weakly similar to T00362 hypotheti | 4.2 |
| 403921 | NA | | C5000212*: gi\|10047237\|dbj\|BAB13407.1\| (A | 4.2 |
| 451477 | AI798425 | Hs.42710 | ESTs | 4.2 |
| 406344 | | | C5001660: gi\|11611537\|dbj\|BAB18935.1\| (AB | 4.2 |
| 416970 | AA191201 | Hs.35861 | DKFZP586E1621 protein | 4.2 |
| 413662 | BE155866 | Hs.25522 | KIAA1808 protein | 4.2 |
| 458504 | AW070634 | Hs.144794 | ESTs | 4.2 |
| 404682 | NA | | C9001188*: gi\|12738842\|ref\|NP_073725.1\|p | 4.2 |
| 418089 | N69913 | Hs.6858 | ESTs, Weakly similar to I78885 serine/th | 4.2 |
| 403433 | | | NM_001622: Homo sapiens alpha-2-HS-glycop | 4.2 |
| 446532 | AW975460 | Hs.143563 | ESTs | 4.2 |
| 414217 | AI309298 | Hs.279898 | Homo sapiens cDNA: FLJ23165 fis, clone L | 4.2 |
| 418425 | AI871247 | Hs.6262 | hypothetical protein MGC8407 | 4.2 |
| 419589 | AW973708 | Hs.201925 | Homo sapiens cDNA FLJ13446 fis, clone PL | 4.2 |
| 457029 | AA397789 | Hs.161803 | ESTs | 4.2 |
| 447860 | AF193807 | Hs.131835 | Rhesus blood group, B glycoprotein | 4.2 |
| 448988 | Y09763 | Hs.22785 | gamma-aminobutyric acid (GABA) A recepto | 4.2 |
| 440610 | AI733098 | Hs.130800 | ESTs | 4.2 |
| 439590 | AF086410 | | gb: Homo sapiens full length insert cDNA | 4.2 |
| 427240 | AA399975 | Hs.274151 | ligatin | 4.2 |
| 408932 | AW594172 | Hs.278513 | TP53TG3 protein | 4.2 |
| 436112 | T77545 | Hs.187559 | ESTs | 4.2 |
| 444382 | AI144152 | Hs.58246 | ESTs | 4.2 |
| 456716 | AA318060 | Hs.135121 | hypothetical protein FLJ22415 | 4.2 |
| 419846 | NM_015977 | Hs.285681 | Williams-Beuren syndrome chromosome regi | 4.2 |
| 410036 | R57171 | Hs.57975 | calsequestrin 2 (cardiac muscle) | 4.1 |
| 400545 | NA | | Target Exon | 4.1 |
| 403051 | NA | | Target Exon | 4.1 |
| 420139 | NM_005357 | Hs.95351 | lipase, hormone-sensitive | 4.1 |
| 450244 | AA007534 | Hs.125062 | ESTs | 4.1 |
| 453261 | AA034116 | Hs.118494 | ESTs | 4.1 |
| 440246 | W52010 | Hs.191379 | ESTs | 4.1 |
| 414516 | AI307802 | Hs.135560 | ESTs, Weakly similar to T43458 hypotheti | 4.1 |
| 438232 | AI150595 | Hs.122226 | ESTs | 4.1 |
| 410233 | AA082947 | | gb: zn10g07.s1 Stratagene hNT neuron (937 | 4.1 |
| 412179 | BE270758 | Hs.69428 | hypothetical protein MGC3020 | 4.1 |
| 441871 | AI306150 | Hs.153450 | ESTs, Weakly similar to 1909123A Na gluc | 4.1 |
| 426411 | AK000708 | Hs.169764 | hypothetical protein FLJ20701 | 4.1 |
| 453692 | AL110416 | | gb: DKFZp434K0431_r1 434 (synonym: htes3) | 4.1 |
| 448640 | AW817177 | Hs.102558 | Homo sapiens, clone MGC: 5352, mRNA, comp | 4.1 |
| 417481 | AA203281 | Hs.21798 | ESTs | 4.1 |
| 412912 | AW118878 | Hs.110835 | ESTs | 4.1 |
| 454183 | AW807116 | | gb: MR4-ST0062-040100-024-b12 ST0062 Homo | 4.1 |
| 426328 | AW631296 | | gb: hh83c09.y1 NCI_CGAP_GU1 Homo sapiens | 4.1 |
| 435942 | R06285 | Hs.191215 | ESTs | 4.1 |
| 417629 | T76945 | | gb: yc92c07.r1 Soares infant brain 1NIB H | 4.1 |
| 403593 | NA | | Target Exon | 4.0 |
| 402690 | | | Target Exon | 4.0 |
| 418190 | R49591 | Hs.270425 | ESTs | 4.0 |
| 408641 | AW245207 | Hs.5555 | hypothetical protein MGC5347 | 4.0 |
| 427899 | AA829286 | Hs.332053 | serum amyloid A1 | 4.0 |
| 445975 | AI811536 | Hs.145734 | ESTs | 4.0 |
| 438831 | BE263273 | Hs.6439 | synapsin II | 4.0 |
| 455578 | BE006350 | Hs.14355 | Homo sapiens cDNA FLJ13207 fis, clone NT | 4.0 |
| 401840 | NA | | Target Exon | 4.0 |
| 413753 | U17760 | Hs.75517 | laminin, beta 3 (nicein (125 kD), kalinin | 4.0 |
| 445030 | AI205925 | Hs.147238 | ESTs, Highly similar to AAC3_HUMAN ALPHA | 4.0 |
| 433873 | AW156913 | Hs.150478 | ESTs, Weakly similar to A Chain A, Cryst | 4.0 |
| 456736 | AW248217 | Hs.1619 | achaete-scute complex (Drosophila) homol | 4.0 |
| 450112 | BE047734 | Hs.5473 | ESTs, Moderately similar to ALU5_HUMAN A | 4.0 |
| 448906 | AI589567 | Hs.309719 | ESTs | 4.0 |
| Table 23 shows 320 genes down-regulated in breast cancer compared to normal adult breast. These were selected as for Table 22, except that the numerator was set to the median value for 12 non-malignant breast specimens, the denominator was set to the median value amongst the 73 breast cancers, | | | | |
| #the 90^{th} percentile value amongst the 12 non-malignant breast specimens was greater than or equal 80 units, and the ratio was greater than or equal to 4.0 (i.e. 4-fold down-regulated in tumor vs. normal breast). | | | | |

Pkey: Unique Eos probeset identifier number
ExAccn: Exemplar Accession number, Genbank accession number
UnigeneID: Unigene number
Unigene Title: Unigene gene title
R1: Ratio of 50^{th} percentile normal body tissue to 75^{th} percentile tumor

TABLE 23A
Table 23A shows the accession numbers for those pkeys lacking
unigeneID's for Table 23. For each probeset, we have listed
the gene cluster number from which the oligonucleotides
were designed. Gene clusters were compiled using sequences
derived from Genbank ESTs and mRNAs. These sequences were
clustered based on sequence similarity using Clustering and
Alignment Tools (DoubleTwist, Oakland California).
The Genbank accession numbers for sequences comprising
each cluster are listed in the “Accession” column.

| Pkey | CAT number | Accessions |
|---|---|---|
| 409853 | 1156226_1 | AW502327 AW502488 AW501829 AW502625 |
| | | AW502687 |
| 410034 | 1170594_1 | BE067414 BE067958 BE067419 BE067963 |
| | | AW577127 AW601412 |
| 410233 | 118656_1 | AA082947 AA083036 |
| 410490 | 1205347_1 | H03589 AW750687 AW750688 |
| 410882 | 1225686_1 | AW809163 AW809247 AW809177 AW809190 |
| | | AW809225 |
| 411478 | 1247073_1 | BE143068 AW849143 AW848705 AW848569 |
| | | AW848071 AW848475 AW848092 AW848005 |
| 413065 | 1347960_1 | BE063555 BE151321 BE151319 BE151657 |
| | | BE151655 BE063556 BE151322 |
| 413072 | 1348163_1 | BE063965 BE063968 BE064034 BE064028 |
| | | BE063874 BE063966 BE063869 BE064043 |
| | | BE064033 BE063884 |
| 414593 | 1464909_1 | BE386764 BE387560 |
| 414913 | 1506721_1 | R25621 C03959 C04010 |
| 415011 | 151328_1 | AW963085 AA159005 AW963073 |
| 415986 | 1564410_1 | Z43619 R61274 H12206 R12883 |
| 416267 | 1583547_1 | H45384 H49125 H41699 |
| 417574 | 1687770_1 | R00348 R09593 |
| 417629 | 1690392_1 | T76945 R20210 R05755 |
| 418556 | 1767866_−1 | T02850 |
| 419583 | 186198_1 | F00312 AA247490 F31427 AA383663 |
| | | F22045 |
| 426328 | 264901_1 | AW631296 AA375484 |
| 439590 | 47413_1 | AF086410 W94386 W74609 |
| 442398 | 541271_1 | AA994520 AW393574 |
| 452205 | 90415_1 | C15819 AA024741 AA024742 |
| 452654 | 925931_1 | BE004783 BE004947 AI911790 |
| 453692 | 977825_1 | AL110416 AW876759 |
| 454183 | 1049636_1 | AW807116 AW807569 AW807415 AW807338 |
| | | AW807288 AW807263 AW807316 AW177402 |
| | | AW807413 AW807068 BE141561 BE141569 |
| | | AW807401 AW807310 BE141565 AW807318 |
| | | AW807119 AW807299 AW807241 AW807225 |
| | | AW807204 AW807345 AW807103 BE141615 |
| | | AW807431 AW807393 AW807337 AW807406 |
| | | AW807259 AW807375 AW845890 AW807220 |
| | | AW807399 AW807064 AW807376 AW807024 |
| | | BE141595 AW807236 AW807027 AW807112 |
| | | AW807378 AW807202 BE141593 AW807216 |
| | | AW807138 AW807244 AW807221 AW807297 |
| | | AW807050 AW807248 AW807404 AW807075 |
| | | AW807237 AW807212 AW807308 AW807110 |
| | | AW807104 BE140912 AW807301 AW807382 |
| | | AW807294 AW807026 AW807020 AW807108 |
| | | AW807025 AW807433 AW807124 AW807419 |
| | | AW807031 AW807264 AW807032 AW807029 |
| | | AW807052 AW807391 AW807207 AW807215 |
| | | AW807019 AW807238 AW807201 BE141590 |
| | | AW807302 AW807323 AW807380 AW807109 |
| | | BE141588 AW845877 AW807418 AW807407 |
| | | AW807309 BE141614 AW845861 AW807396 |
| | | AW807300 AW807348 AW807311 AW807214 |
| | | AW807132 AW807402 AW807350 AW807028 |
| | | AW807298 AW807291 AW807305 AW807217 |
| | | AW807312 AW807261 AW807268 AW807260 |
| | | AW807266 AW807198 AW807346 AW807315 |
| | | BE140940 AW807351 AW807122 AW807235 |
| | | AW807076 AW807314 AW807213 AW807143 |
| | | AW807131 AW800079 AW807066 AW807077 |
| | | AW845923 AW807421 BE141619 BE140943 |
| | | AW807420 AW807381 BE141621 AW845921 |
| | | BE141629 BE141625 BE141624 BE141636 |
| | | BE141630 AW807405 AW807290 AW807353 |
| 454404 | 1170594_1 | BE067414 BE067958 BE067419 BE067963 |
| | | AW577127 AW601412 |
| 454775 | 1234106_1 | BE160229 AW819879 AW820179 AW819882 |
| | | AW819876 AW820169 BE153201 AW993736 |
| | | BE152911 |
| 455282 | 1273020_1 | BE143867 AW935060 AW886684 |
| 459159 | 919998_1 | AI904646 BE179494 BE179421 |

Pkey: Unique Eos probeset identifier number
CAT number: Gene cluster number
Accession: Genbank accession numbers

TABLE 23B
Table 23B shows the genomic positioning for those pkeys
lacking unigene ID's and accession numbers in Table 23.
For each predicted exon, we have listed the genomic
sequence source used for prediction. Nucleotide locations of
each predicted exon are also listed.

| Pkey | Ref | Strand | Nt_position |
|---|---|---|---|
| 400545 | 9800107 | Minus | 124618-124881 |
| 400870 | 9838306 | Minus | 34081-35027 |
| 400973 | 7960452 | Minus | 98119-98253 |
| 401093 | 8516137 | Minus | 22335-23166 |
| 401590 | 9966320 | Minus | 33547-33649 |
| 401665 | 7145001 | Plus | 121591-122537 |
| 401810 | 7342191 | Plus | 129063-129476 |
| 401840 | 7684597 | Plus | 56283-56439 |
| 402054 | 8083691 | Minus | 8288-8806 |
| 402195 | 7689778 | Minus | 147901-148884 |
| 402583 | 7684486 | Plus | 94883-95003 |
| 402690 | 8348058 | Plus | 13368-13998 |
| 402698 | 8570304 | Minus | 108641-108903 |
| 402779 | 9588555 | Minus | 38173-39210 |
| 403017 | 6693623 | Plus | 78630-79367 |
| 403051 | 4827080 | Minus | 5269-5411 |
| 403263 | 7770677 | Plus | 52431-52737 |
| 403433 | 9719611 | Minus | 72225-72437 |
| 403593 | 6862650 | Minus | 62554-62712, 69449-69602 |
| 403612 | 8469060 | Minus | 94723-94859 |
| 403921 | 7711590 | Minus | 3297-3536 |
| 404368 | 7630956 | Minus | 102053-102199 |
| 404682 | 9797231 | Minus | 40977-41150 |
| 404689 | 7534100 | Plus | 119461-119717 |
| 405016 | 6524300 | Plus | 51997-53308 |
| 405062 | 7657730 | Plus | 101283-101432 |
| 406118 | 9143818 | Plus | 53997-54629 |
| 406344 | 9255974 | Plus | 20254-20374, 20526-20659, |
| | | | 20835-21097 |
| 406563 | 7711604 | Plus | 34401-34538 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 24
Table 24 depicts Seq ID No., UnigeneID, UnigeneTitle, Pkey, Pred.Cell.Loc., and ExAccn for all
of the sequences in Table 25. The information in Table 24 is linked by Seq ID No. to Table 25.

| Pkey | ExAccn | UnigeneID | Unigene Title | Pred. Cell. Loc. | Seq. ID. No. |
|---|---|---|---|---|---|
| 449746 | AI668594 | Hs.176588 | ESTs, Weakly similar to CP4Y_HUMAN CYTOC | | Seq ID 1 & 2 |
| 407276 | AI951118 | Hs.326736 | Homo sapiens breast cancer antigen NY-BR | | Seq ID 3 & 4 |
| 415539 | AI733881 | Hs.72472 | BMP-R1B | | Seq ID 5 & 6 |
| 400297 | AI127076 | Hs.334473 | hypothetical protein DKFZp564O1278 | | Seq ID 7 & 8 |
| 450375 | AA009647 | Hs.8850 | a disintegrin and metalloproteinase doma | | Seq ID 9 & 10 |
| 102457 | NM_001394 | Hs.2359 | dual specificity phosphatase 4 | nuclear | Seq ID 11 & 12 |
| 429170 | NM_001394 | Hs.2359 | dual specificity phosphatase 4 | nuclear | Seq ID 11 & 12 |
| 424399 | AI905687 | Hs.2533 | aldehyde dehydrogenase 9 family, member | cytoplasm | Seq ID 13 & 14 |
| 422505 | AL120862 | Hs.124165 | ESTs | | Seq ID 15 & 16 |
| 449765 | N92293 | Hs.206832 | ESTs, Moderately similar to ALU8_HUMAN A | | Seq ID 17 & 18 |
| 425692 | D90041 | Hs.155956 | N-acetyltransferase 1 (arylamine N-acety | | Seq ID 19 & 20 |
| 426215 | AW963419 | Hs.155223 | stanniocalcin 2 | | Seq ID 21 & 22 |
| 439840 | AW449211 | Hs.105445 | GDNF family receptor alpha 1 | | Seq ID 23 & 24 |
| 410102 | AW248508 | Hs.279727 | Homo sapiens cDNA FLJ14035 fis, clone HE | | Seq ID 25 & 26 |
| 429220 | AW207206 | Hs.136319 | ESTs | | Seq ID 27 & 28 |
| 416276 | U41060 | Hs.79136 | LIV-1 protein, estrogen regulated | | Seq ID 29 & 30 |
| 409079 | W87707 | Hs.82065 | interleukin 6 signal transducer (gp130, | | Seq ID 31 & 32 |
| 442818 | AK001741 | Hs.8739 | hypothetical protein FLJ10879 | | Seq ID 33 & 34 |
| 442082 | R41823 | Hs.7413 | ESTs | | Seq ID 35 & 36 |
| 444381 | BE387335 | Hs.283713 | ESTs, Weakly similar to S64054 hypotheti | | Seq ID 37 & 38 |
| 446163 | AA026880 | Hs.25252 | Homo sapiens cDNA FLJ13603 fis, clone PL | | Seq ID 39 & 40 |
| 416636 | N32536 | Hs.42645 | solute carrier family 16 (monocarboxylic | | Seq ID 41 & 42 |
| 442117 | AW664964 | Hs.128899 | ESTs | | Seq ID 43 & 44 |
| 433043 | W57554 | Hs.125019 | lymphoid nuclear protein (LAF-4) mRNA | | Seq ID 45 & 46 |
| 429353 | AL117406 | Hs.200102 | ATP-binding cassette transporter MRP8 | | Seq ID 47 & 48 |
| 452190 | H26735 | Hs.91668 | Homo sapiens clone PP1498 unknown mRNA | | Seq ID 49 & 50 |
| 446733 | AA863360 | Hs.26040 | ESTs, Weakly similar to fatty acid omega | | Seq ID 51 & 52 |
| 452747 | BE153855 | Hs.61460 | Ig superfamily receptor LNIR | | Seq ID 53 & 54 |
| 423242 | AL039402 | Hs.125783 | DEME-6 protein | | Seq ID 55 & 56 |
| 417433 | BE270266 | Hs.82128 | 5T4 oncofetal trophoblast glycoprotein | | Seq ID 57 & 58 |
| 432201 | AI538613 | Hs.298241 | Transmembrane protease, serine 3 | | Seq ID 59 & 60 |
| 423961 | D13666 | Hs.136348 | osteoblast specific factor 2 (fasciclin | | Seq ID 61 & 62 |
| 439569 | AW602166 | Hs.222399 | CEGP1 protein | | Seq ID 63 & 64 |
| 114480 | BE066778 | Hs.151678 | UDP-N-acetyl-alpha-D-galactosamine: polyp | | Seq ID 65 & 66 |
| 404561 | | | NM_014112*: Homo sapiens trichorhinophala | mitochodria | Seq ID 67 & 68 |
| 325372 | NA | | Phase 2 & 3 Exons | nuclear | Seq ID 69 & 70 |
| 112287 | AB033064 | Hs.334806 | KIAA1238 protein | | Seq ID 71 & 72 |
| 335824 | NA | | ENSP00000249072*: DJ222E13.1 (N-TERMINAL | | Seq ID 73 & 74 |
| 424735 | U31875 | Hs.272499 | short-chain alcohol dehydrogenase family | | Seq ID 75 & 76 |
| 400289 | X07820 | Hs.2258 | matrix metalloproteinase 10 (stromelysin | | Seq ID 77 & 78 |
| 427585 | D31152 | Hs.179729 | collagen, type X, alpha 1 (Schmid metaph | | Seq ID 79 & 80 |
| 429925 | NM_000786 | Hs.226213 | cytochrome P450, 51 (lanosterol 14-alpha | ER | Seq ID 81 & 82 |
| 429441 | AJ224172 | Hs.204096 | lipophilin B (uteroglobin family member) | | Seq ID 83 & 84 |
| 421155 | H87879 | Hs.102267 | lysyl oxidase | extracellular | Seq ID 85 & 86 |
| 420931 | AF044197 | Hs.100431 | small inducible cytokine B subfamily (Cy | | Seq ID 87 & 88 |
| 420813 | X51501 | Hs.99949 | prolactin-induced protein | nuclear | Seq ID 89 & 90 |
| 452744 | AI267652 | Hs.30504 | Homo sapiens mRNA; cDNA DKFZp434E082 (fr | | Seq ID 91 & 92 |
| 420757 | X78592 | Hs.99915 | androgen receptor (dihydrotestosterone r | cytoplasm | Seq ID 93 & 94 |
| 424905 | NM_002497 | Hs.153704 | NIMA (never in mitosis gene a)-related k | nuclear | Seq ID 95 & 96 |
| 429859 | NM_007050 | Hs.225952 | protein tyrosine phosphatase, receptor t | | Seq ID 97 & 98 |
| 446921 | AB012113 | Hs.16530 | small inducible cytokine subfamily A (Cy | extracellular | Seq ID 99 & 100 |
| 445537 | AJ245671 | Hs.12844 | EGF-like-domain, multiple 6 | | Seq ID 101 & 102 |
| 428227 | AA321649 | Hs.2248 | small inducible cytokine subfamily B (Cy | extracellular | Seq ID 103 & 104 |
| 424001 | W67883 | Hs.137476 | paternally expressed 10 | | Seq ID 105 & 106 |
| 421727 | Y13153 | Hs.107318 | kynurenine 3-monooxygenase (kynurenine 3 | | Seq ID 107 & 108 |
| 452838 | U65011 | Hs.30743 | preferentially expressed antigen in mela | nuclear | Seq ID 109 & 110 |
| 419667 | AU077005 | Hs.92208 | a disintegrin and metalloproteinase doma | | Seq ID 111 & 112 |
| 414812 | X72755 | Hs.77367 | monokine induced by gamma interferon | extracellular | Seq ID 113 & 114 |
| 426320 | W47595 | Hs.169300 | transforming growth factor, beta 2 | extracellular | Seq ID 115 & 116 |
| 422867 | L32137 | Hs.1584 | cartilage oligomeric matrix protein (pse | extracellular | Seq ID 117 & 118 |
| 411558 | AA102670 | Hs.70725 | gamma-aminobutyric acid (GABA) A recepto | | Seq ID 119 & 120 |
| 417866 | AW067903 | Hs.82772 | collagen, type XI, alpha 1 | | Seq ID 121 & 122 |
| 428398 | AI249368 | Hs.98558 | ESTs | | Seq ID 123 & 124 |
| 431958 | X63629 | Hs.2877 | cadherin 3, type 1, P-cadherin (placenta | plasma membrane | Seq ID 125 & 126 |
| 428722 | U76456 | Hs.190787 | tissue inhibitor of metalloproteinase 4 | | Seq ID 127 & 128 |
| 412970 | AB026436 | Hs.177534 | dual specificity phosphatase 10 | | Seq ID 129 & 130 |
| 421379 | Y15221 | Hs.103982 | small inducible cytokine subfamily B (Cy | extracellular | Seq ID 131 & 132 |
| 415752 | BE314524 | Hs.78776 | putative transmembrane protein | | Seq ID 133 & 134 |
| 444051 | N48373 | Hs.10247 | activated leucocyte cell adhesion molecu | | Seq ID 135 & 136 |
| 451110 | AI955040 | Hs.265398 | ESTs, Weakly similar to transformation-r | | Seq ID 137 & 138 |

Pkey: Unique Eos probeset identifier number
ExAccn: Exemplar Accession number, Genbank accession number
UnigeneID: Unigene number
Unigene Title: Unigene gene title
Pred. Cell. Loc.: Predicted Cellular Localization
Seq. ID. No.: Sequence Identification Number found in Table 25

TABLE 24A
Table 24A shows the accession numbers for those pkeys
lacking unigeneID's for Table 24. For each probeset, we
have listed the gene cluster number from which the
oligonucleotides were designed. Gene clusters were compiled
using sequences derived from Genbank ESTs and mRNAs.
These sequences were clustered based on sequence
similarity using Clustering and Alignment Tools
(DoubleTwist, Oakland California). The Genbank accession
numbers for sequences comprising each cluster are
listed in the “Accession” column.

| | | |
|---|---|---|
| Pkey | CAT number | Accession |

| | |
|---|---|
| 335824 | CH22_3197FG_619_11_LINK_E |
| 325372 | c12_hs |

Pkey: Unique Eos probeset identifier number
CAT number: Gene cluster number
Accession: Genbank accession numbers

TABLE 24B
Table 24B shows the genomic positioning for those pkeys
lacking unigene ID's and accession numbers in Table 24.
For each predicted exon, we have listed the genomic
sequence source used for prediction. Nucleotide
locations of each predicted exon are also listed.

| Pkey | Ref | Strand | Nt_position |
|---|---|---|---|
| 404561 | 9795980 | Minus | 69039-70100 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22.” Dunham I. et al., Nature (1999) 402: 489-495.
Strand: Indicates DNA strand from which exons were predicted.
Nt_position: Indicates nucleotide positions of predicted exons.

TABLE 25

The 69 gene sequences identified to be overexpressed in breast cancer
may be used to identify coding regions from the public DNA databases
(nr and htgs in Genbank). The sequences may be used to either identify
genes that encode known proteins, or they may be used to predict the
coding regions from genomic DNA using exon prediction algorithms,
sueh as FGENESH (Sstamov and Solovyev, 2000, Genome Rca. 10:516-522)

Seq ID NO: 1 DNA sequence
Nucleic Acid Accession #: FGENESH predicted ORF
Coding sequence: 1-1518 (underlined sequences correspond to start and
stop codons)

| | |
|---|---|
| 1 11 21 31 41 51 | |
| \| \| \| \| \| \| | |
| ATGGAGCCCT CCTGGCTTCA GGAACTCATG GCTCACCCCT TCTTGCTGCT GATCCTCCTC | 60 |
| TGCATGTCTC TGCTGCTGTT TCAGGTAATC AGGTTGTACC AGAGGAGGAG ATGGATGATC | 120 |
| AGAGCCCTGC ACCTGTTTCC TGCACCCCCT GCCCACTGGT TCTATGGCCA CAAGGAGTTT | 180 |
| TACCCAGTAA AGGAGTTTGA GGTGTATCAT AAGCTGATGG AAAAATACCC ATGTGCTGTT | 240 |
| CCCTTGTGGG TTGGACCCTT TACGATGTTC TTCAGTGTCC ATGACCCAGA CTATGCCAAG | 300 |
| ATTCTCCTGA AAAGACAAGA TCCCAAAAGT GCTGTfAGCC ACAAAATCCT TGAATCCTGG | 360 |
| GTTGGTCGAG GACTTGTGAC CCTGGATGGT TCTAAATGGA AAAAGCACCG CCAGATTGTG | 420 |
| AAACCTGGCT TCAACATCAG CATTCTGAAA ATATTCATCA CCATGATGTC TGAGAGTGTT | 480 |
| CGGATGATGC TGAACAAATG GGAGGAACGC ATTGCCCAAA ACTCACGTCT GGAGCTCTTT | 540 |
| CAACATGTCT CCCTGATGAC CCTGGACAGC ATCATGAAGT GTGCCTCCAG CCACCAGGGC | 600 |
| AGCATCCAGT TGGACAGTAC CCTGGACTCA TACCTGAAAG CAGTGTTCAA CCTTAGCAAA | 660 |
| ATCTCCAACC AGCGCATGAA CAATTTfCTA CATCACAACG ACCTGGTTTT CAAATTCAGC | 720 |
| TCTCAAGGCC AAATCTTTTC TAAATTTAAC CAAGAACTTC ATCAGTTCAC AGAGAAAGTA | 780 |
| ATCCAGGACC GGAAGGAGTC TCTTAAGGAT AAGCTAAAAC AAGATACTAC TCAGAAAAGG | 840 |
| CGCTGGGATT TTCTGGACAT ACTTTTOAGT GCCAAAAGCG AAAACACCAA AGATTTCTCT | 900 |
| GAAGCAGATC TCCAGGCTGA AGTGAAAACG TTCATGTTTG CAGGACATGA CACCACATCC | 960 |
| AGTGCTATCT CCTGGATCCT TTACTGCTTG GCAAAGTACC CTGAGCATCA GCAGAGATGC | 1020 |
| CGAGATGAAA TCAGGGAACT CCTAGGGGAT GGGTCTTCTA TTACCTGGGA ACACCTGAGC | 1080 |
| CAGATGCCTT ACACCACGAT GTGCATCAAG GAATGCCTCC GCCTCTACGC ACCGGTAGTA | 1140 |
| AACATATCCC GGTTACTCGA CAAACCCATC ACCTTTCCAG ATGGACGCTC CTTACCTGCA | 1200 |
| GGAATAACTG TGTTTATCAA TATTTGGGCT CTTCACCACA ACCCCTATTT CTGGGAAGAC | 1260 |
| CCTCAGGTCT TTAACCCCTT GAGATTCTCC AGGGAAAATT CTGAAAAAAT ACATCCCTAT | 1320 |
| GCCTTCATAC CATTCTCAGC TGGATTAAGG AACTGCATTG GGCAGCATTT TGCCATAATT | 1380 |
| GAGTGTAAAG TGGCAGTGGC ATTAACTCTG CTCCGCTTCA AGCTGGCTCC AGACCACTCA | 1440 |
| AGGCCTCCCC AGCCTGYfCG TCAAGTTGTC CTCAAGTCCA AGAATGGAAT CCATGTGTTT | 1500 |
| GCAAAAAAAG TTTGCTAATT TTAAGTCCTT TCGTATAAGA ATTAATGAGA CAATTTTCCT | 1560 |
| ACCAAAGGAA GAACAAAAGG ATAAATATAA TACAAAATAT ATGTATATGG TTGTTTGACA | 1620 |
| AATTATATAA CTTAGGATAC TTCTGACTGG TTTTGACATC CATTAACAGT AATTTTAATT | 1680 |
| TCTTTGCTGT ATCTGGTGAA ACCCACAAAA ACACCTGAAA AAACTCAAGC TGACTTCCAC | 1740 |
| TGCGAAGGGA AATTATTGGT TTGTGTAACT AGTGGTAGAG TGGCTTTCAA GCATAGTTTG | 1800 |
| ATCAAAACTC CACTCAGTAT CTGCATTACT TTTATCTCTG CAAATATCTG CATGATAGCT | 1860 |
| TTATTCTCAG TTATCTTTCC CCAATAATAA AAAA | |

Seq ID NO: 2 Protein sequence:
Protein Accession #: FGENESH predicted

| | |
|---|---|
| 1 11 21 31 41 51 | |
| \| \| \| \| \| \| | |
| MEPSWLQELM AHPFLLLILL CMSLLLFQVI RLYQRRRWMI RALHLFPAPP AHWFYGHKEF | 60 |
| YPVKEFEVYH KLMEKYPCAV PLWVGPFTMF FSVHDPDYAK ILLKRQDPKS AVSHKILESW | 120 |
| VGRGLVTLDG SKWKKHRQIV KPGFNISILK IFITMMSESV RMMLNKWEER IAQNSRLELF | 180 |
| QHVSLMTLDS IMKCAFSHQG SIQLDSTLDS YLKAVFNLSK ISNQRMNNFL HHNDLVFKFS | 240 |
| SQGQIFSKFN QELHQETEKV IQDRKESLKD KLKQDTTQKR RWDFLDILLS AXSENTKDFS | 300 |
| EADLQAEVKT FMFAGHDTTS SAISWILYCL AKYPEHQQRC RDEIRELLGD GSSITWEHLS | 360 |
| QMPYTTMCIK ECLRLYAPVV NISRLLDKPI TFPDGRSLPA GITVFINIWA LHHNPYFWED | 420 |
| PQVFNPLRFS RENSEKIHPY AFIPFSAGLR NCIGQHFAII ECKVAVALTL LRFKLAPDHS | 480 |
| RPPQPVRQVV LKSKNGIHVF AKKVC | |

Seq ID NO: 3 DNA sequence
Nucleic Acid Accession #: NM_052997
Coding sequence: 100-4125 (underlined sequences correspond to start
and stop codons)

| | |
|---|---|
| 1 11 21 31 41 51 | |
| \| \| \| \| \| \| | |
| CTAGTCTATA CCAGCAACGA CTCCTACATC GTCCACTCTG GGGATCTTAG AAAGATCCAT | 60 |
| AAAGCTGCCT CCCGGGGACA AGTCCGGAAG CTGGAGAAGA TGACAAAGAG GAAGAAGACC | 120 |
| ATCAACCTTA ATATACAAGA CGCCCAGAAG AGGACTGCTC TACACTGGGC CTGTGTCAAT | 180 |
| GGCCATGAGG AAGTAGTAAC ATTTCTGGTA GACAGAAAGT GCCAGCTTGA CGTCCTTGAT | 240 |
| GGCGAACACA GGACACCTCT GATGAAGGCT CTACAATGCC ATCAGGAGGC ITGTGCAAAT | 300 |
| ATrCTGATAG ATTCTGGTGC CGATATAAAT CTCGTAGATO TGTATGGCAA CATGGCTCTC | 360 |
| CATTATGCTG TTTATAGTGA GATTTfGTCA GTGGTGGCAA AACTGCTGTC CCATGGTGCA | 420 |
| GTCATCGAAG TGCACAACAA GGCTAGCCTC ACACCACTTT TACTATCCAT AACGAAAAGA | 480 |
| AGTGAGCAAA TTGTGGAATT TTTGCTGATA AAAAATGCAA ATGCGAATGC AGTTAATAAG | 540 |
| TATAAATGCA CAGCCCTCAI GCTTGCTGTA TGTCATGGAT CATCAGAGAT AGTTGGCATG | 600 |
| CTTCTTCAGC AAAATGTTGA CGTCTTTGCT GCAGATATAT GTGGAGTAAC TGCAGAACAT | 660 |
| TATGCTGTTA CTTGTGGATT TCATCACATT CATGAACAAA TTATGGAATA TATACGAAAA | 720 |
| TTATCTAAAA ATCATCAAAA TACCAATCCA GAAGGAACAT CTGCAGGAAC ACCTGATGAG | 780 |
| GCTGCACCCT TGGCGGAAAG AACACCTGAC ACAGCTGAAA GCTTGGTGGA AAAAACACCT | 840 |
| GATGAGGCTG CACCCTTGGT GGAAAGAACA CCTGACACGG CTGAAAGCTT GGTGGAAAAA | 900 |
| ACACCTGATG AGGCTGCATC CTTGGTGGAG GGAACATCTG ACAAAATTCA ATGTTTGGAG | 960 |
| AAAGCGACAT CTGGAAAGTT CGAACAGTCA GCAGAAGAAA CACCTAGGGA AATTACGAGT | 1020 |
| CCTGCAAAAG AAACATCTGA GAAATTTACG TGGCCAGCAA AAGGAAGACC TAGGAAGATC | 1080 |
| GCATGGGAGA AAAAAGAAGA CACACCTAGG GAAATTATGA GTCCCGCAAA AGAAACATCT | 1140 |
| GAGAAATTTA CGTGGGCAGC AAAAGGAAGA CCTAGGAAGA TCGCATGGGA GAAAAAAGAA | 1200 |
| ACACCTGTAA AGACTGGATG CGTGGCAAGA GTAACATCTA ATAAAACTAA AGTTTTGGAA | 1260 |
| AAAGGAAGAT CTAAGATGAT TGCATGTCCT ACAAAAGAAT CATCTACAAA AGCAAGTGCC | 1320 |
| AATGATCAGA GGTTCCCATC AGAATCCAAA CAAGAGGAAG ATGAAGAATA TTCTTGTGAT | 1380 |
| TCTCGGAGTC TCTTTGAGAG TTCTGCAAAG ATTCAAGTGT GTATACCTGA GTCTATATAT | 1440 |
| CAAAAAGTAA TGGAGATAAA TAGAGAAGTA GAAGAGCCTC CTAAGAAGCC ATCTGCCTTC | 1500 |
| AAGCCTGCCA TTGAAATGCA AAACTCTG3T CCAAATAAAG CCTTTGAATT GAAGAATQAA | 1560 |
| CAAACATTGA GAGCAGATCC GATGTTCCCA CCAGAATCCA AACAAAAGGA CTATGAAGAA | 1620 |
| AATTCTTGGG ATTCTGAGAG TCTCTGTGAG ACTGTTTCAC AGAAGGATGT GTGTTTACCC | 1680 |
| AAGGCTACAC ATCAAAAAGA AATAGATAAA ATAAATGGAA AATTAGAAGA GTCTCCTAAT | 1740 |
| AAAGATGGTC TTCTGAAGGC TACCTGCGGA ATGAAAGTTT CTATTCCAAC TAAAGCCTTA | 1800 |
| GAATTGAAGG ACATGCAAAC TTTCAAAGCG GAGCCTCCGG GGAAGCCATC TGCCTTCGAG | 1860 |
| CCTGCCACTG AAATGCAAAA GTCTGTCCCA AATAAAGCCT TGGAATTGAA AAATGAACAA | 1920 |
| ACATGGAGAG CAGATGAGAT ACTCCCATCA GAATCCAAAC AAAAGGACTA TGAAGAAAAT | 1980 |
| TCTTGGGATA CTGAGAGTCT CTGTGAGACT GTTTCACAGA AGGATGTGTG GTTACCCAAG | 2040 |
| GCTGCGCATC AAAAAGAAAT AGATAAAATA AATGGAAAAT TAGAAGGGTC TCCTGTTAAA | 2100 |
| GATGGTCTTC TGAAGGCTAA CTGCGGAATG AAAGTTTCTA TTCCAACTAA AGCCTTAGAA | 2160 |
| TTGATGGACA TGCAAACTTT CAAAGCAGAG CCTCCCGAGA AGCCATCTGC CTTCGAGCCT | 2220 |
| GCCATTGAAA TGCAAAAGTC TGTTCCAAAT AAAGCCTTGG AATTGAAGAA TGAACAAACA | 2280 |
| TTGAGAGCAG ATGAGATACT CCCATCAGAA TCCAAACAAA AGGACTATGA AGAAAGTTCT | 2340 |
| TGGGATTCTG AGAGTCTCTG TGAGACTGTT TCACAGAAGG ATGTGTGTTT ACCCAAGGCT | 2400 |
| ACACATCAAA AAGAAATAGA TAAAATAAAT GGAAAATTAG AAGAGTCTCC TGATAATGAT | 2460 |
| GGTTTTCTGA AGGCTCCCTG CAGAATGAAA GTTGCTATTC CAACTAAAGC CTTAGAATTG | 2520 |
| ATGGACATGC AAACTTTCAA AGCAGAGCCT CCCGAGAAGC CATCTGCCTT CGAGCCTGCC | 2580 |
| ATTGAAATGC AAAAGTCTGT TCCAAATAAA GCCTTGGAAT TGAAGAATGA ACAAACATTG | 2640 |
| AGAGCAGATC AGATGTTCCC TTCAGAATCA AAACAAAAGA AGGTTGAAGA AAATTCTTGG | 2700 |
| GATTCTGAGA GTCTCCGTGA GACTGTTTCA CAGAAGGATG TGTGTGTACC CAAGGCTACA | 2760 |
| CATCAAAAAG AAATGGATAA AATAAOTGGA AAATTAGAAG ATTCAACTAG CCTATCAAAA | 2820 |
| ATCTTGGATA CAGTTCATTC TTGTGAAAGA GCAAGGGAAC TTCAAAAAGA TCACTGTGAA | 2880 |
| CAACGTACAG GAAAAATGGA ACAAATGAAA AAGAAGTTTT GTGTACTGAA AAAGAAACTG | 2940 |
| TCAGAAGCAA AAGAAATAAA ATCACAGTTA GAGAACCAAA AAGTTAAATG GGAACAAGAG | 3000 |
| CTCTGCAGTG TGAGATTGAC TITAAACCAA GAAGAAGAGA AGAGAAGAAA TGCCGATATA | 3060 |
| TTAAATGAAA AAATTAGGGA AGAATTAGGA AGAATCGAAG AGCAGCATAG GAAAGAGTTA | 3120 |
| GAAGTGAAAC AACAACTTGA ACAGGCTCTC AGAATACAAG ATATAGAATT GAAGAGTGTA | 3180 |
| GAAAGTAATT TGAATCAGGT TTCTCACACT CATGAAAATG AAAATTATCT CTTACATGAA | 3240 |
| AATTGCATGT TGAAAAAGGA AATTGCCATG CTAAAACTGG AAATAGCCAC ACTGAAACAC | 3300 |
| CAATACCAGG AAAAGGAAAA TAAATACITT GAGGACATTA AGATTTTAAA AGAAAAGAAT | 3360 |
| GCTGAACTTC AGATGACCCT AAAACTGAAA GAGGAATCAT TAACTAAAAG GGCATCTCAA | 3420 |
| TATAGTGGGC AGCTTAAAGT TCTGATAGCT GAGAACACAA TGCTCACTTC TAAATTGAAG | 3480 |
| GAAAAACAAG ACAAAGAAAT ACTAGAGGCA GAAATTGAAT CACACCATCC TAGACTGGCT | 3540 |
| TCTGCTGTAC AAGACCATGA TCAAATGGTG ACATCAAGAA AAAGTCAAGA ACCTGCTTTC | 3600 |
| CACATTOCAG GAGATGCTTG TTTGCAAAGA AAAATGAATG TTGATGTGAG TAGTACGATA | 3660 |
| TATAACAATG AGGTGCTCCA TCAACCACTT TCTGAAGCTC AAAGGAAATC CAAAAGCCTA | 3720 |
| AAAATTAATC TCAATTATGC AGGAGATGCT CTAAGAGAAA ATACATTGGT TTCAGAACAT | 3780 |
| GCACAAAGAG ACCAACGTGA AACACAGTGT CAAATGAAGG AAGCTGAACA CATGTATCAA | 3840 |
| AACGAACAAG ATAATGTGAA CAAACACACT GAACAGCAGG AGTCTCTAGA TCAGAAATTA | 3900 |
| TTTCAACTAC AAAGCAAAAA TATGTGGCTT CAACAGCAAT TAGTTCATGC ACATAAGAAA | 3960 |
| GCTGACAACA AAAGCAAGAT AACAATTGAT ATTCATTTTC TTGAGAGGAA AATGCAACAT | 4020 |
| CATCTCCTAA AAGAGAAAAA TGAGGAGATA TTTAATTACA ATAACCATTT AAAAAACCGT | 4080 |
| ATATATCAAT ATGAAAAAGA GAAAGCAGAA ACAGAAAACT CATGAGAGAC AAGCAGTAAG | 4140 |
| AAACTTCTTT TGGAGAAACA ACAGACCAGA TCTTTACTCA CAACTCATGC TAGGAGGCCA | 4200 |
| GTCCTAGCAT CACCTTATGT TGAAAATCTT ACCAATAGTC TGTGTCAACA GAATACTTAT | 4260 |
| TTTAGAAGAA AAATTCATGA TTTCTTCCTG AAGCCTACAG ACATAAAATA ACAGTGTGAA | 4320 |
| GAATTACTTG TTCACGAATT GCATAAAGCT GCACAGGA3T CCCATCTACC CTGATGATGC | 4380 |
| AGCAGACATC ATTCAATCCA ACCAGAATCT CGCTCTGCAC TCCAGCCTAG GTGACAGAGT | 4440 |
| GAGACTCCAC CTCGGAAA | |

Seq ID NO: 4 Protein sequence:
Protein Accession #: NP_443723.1

| | |
|---|---|
| 1 11 21 31 41 51 | |
| \| \| \| \| \| \| | |
| MTKRKKTINL NIQDAQKRTA LHWACVNGHE EVVTFLVDRK CQLDVLDGEH RTPLMKALQC | 60 |
| HQEACANILI DSGADINLVD VYGNMALHYA VYSEILSVVA KLLSHGAVIE VHNKASLTPL | 120 |
| LLSITKRSEQ IVEFLLIKNA NANAVNXYKC TALMLAVCHG SSEIVGMLLQ QNVDVFAADI | 80 |
| CGVTAEHYAV TCGFHHIHEQ IMEYIRKLSK NHQNTNPEGT SAGTPDEAAP LAERTPDTAE | 240 |
| SLVEKTPDEA APLVERTPDT AESLVEKTPD EAASLVEGTS DKIQCLEKAT SGKFEQSAEE | 300 |
| TPREITSPAK ETSEKFTWPA KGRFRKIAWE KKEDTPREIM SPAKETSEKF TWAAXGRPRK | 360 |
| IAWEKKETPV KTGCVARVTS NKTKVLEKGR SKMIACPTKE SSTKASANDQ RFPSESKQEE | 420 |
| DEEYSCDSRS LFESSAKIQV CIPESIYQKV MEINREVEEP PKKPSAFKPA IEMQNSVPNK | 480 |
| AFELKNEQTL RADPMFPPES KQKDYEENSW DSESLCETVS QKDVCLPKAT HQKEIDKJNG | 540 |
| KLEESPNKDG LLKATCGMKV SIPTKALELK DMQTFKAEPP GKPSAFEPAT EMQKSVPNKA | 600 |
| LELKNEQTWR ADEILPSESK QKDYEENSWD TESLCETVSQ KDVCLPKAAH QKEIDKINGK | 660 |
| LEGSPVKDGL LKANCGMKVS IPTKALELMD MQTFKAEPPE KPSAFEPAIE MQKSVPNKAL | 720 |
| ELKNEQTLRA DEILPSESKQ KDYEESSWDS ESLCETVSQK DVCLPKATHQ KEIDKNGKL | 780 |
| EESPDNDGFL KAPCRMKVSI PTKALELMDM QTFKAEPPEK PSAFEPAIEM QKSVPNKALE | 840 |
| LKNEQTLRAD QMFPSESKQK KVEENSWDSE SLRETVSQKD VCVPKATHQK EMDKISGKLE | 900 |
| DSTSLSK1LD TVHSCEIARE LQKDHCEQRT GKMEQMKKKF CVLKXKLSEA KEIKSQLENQ | 960 |
| KVKWEQELCS VRLTLNQEEE ERENADILNE KIREELGRIE EQHRKELEVK QQLEQALRIQ | 1020 |
| DIELKSVESN LNQVSHTHEN ENYLLIENCM LKKEIAMLKL EIATLKHQYQ EKENKYFEDI | 1080 |
| KILKEKNAEL QMTLKLKEES LTKRASQYSG QLKVLIAENT MLTSKLKEKQ DKEILEAEIE | 1140 |
| SHHPRLASAV QDHDQIVTSR KSQEPAFHIA GDACLQRKMN VDVSSTIYNN EVLHQPLSEA | 1200 |
| QRKSKSLKIN LNYAGDALRE NTLVSEHAQR DQRETQCQMK EAEHMYQNEQ DNVNKHTEQQ | 1260 |
| ESLDQKLFQL QSKNMWLQQQ LVHAHXKADN KSKITIDIHF LERXMQHHLL KEKNEEIFNY | 1320 |
| NNHLKNRTYQ YEKEKAETEN S | |

Seq ID NO: 5 DNA sequece
Nucleic Acid Accession #: none found
Coding sequence: 273-1785 (underlined sequences correspond to start
and stop codons)

| | |
|---|---|
| 1 11 21 31 41 51 | |
| \| \| \| \| \| \| | |
| CGCGGGGCGC GGAGTCGGCG GGGCCTCGCG GGACGCGGGC AGTGCGGAGA CCGCGGCGCT | 60 |
| GAGGACGCGG GAGCCGGGAG CGCACGCGCG GGGTGGAGTT CAGCCTACTC TTTCTTAGAT | 120 |
| GTGAAAGGAA AGGAAGATCA TTTCATGCCT TGTTGATAAA GGTTCAGACT TCTGCTGATT | 180 |
| CATAACCATT TGGCTCTGAG CTATGACAAG AGAGGAAACA AAAAOTTAAA CTTACAAGCC | 240 |
| TGCCATAAGT GAGAAGCAAA CTTCCTTGAT AACATGCT3T TGCGAAGTGC AGGAAAATTA | 300 |
| AATGTGGGCA CCAAGAAAGA GGATGGTGAG AGTACAGCCC CCACCCCCCG TCCAAAGGTC | 360 |
| TTGCGTTGTA AATGCCACCA CCATTGTCCA OAAGACTCAG TCAACAATAT TTGCAGCACA | 420 |
| GACGGATATT G1TTCACGAT GATAGAAGAG GATGACTCTG GGTTGCCTGT GGTCACTTCT | 480 |
| GGTTGCCTAG GACTAGAAGG CTCAGATTTT CAGTGTCGGG ACACTCCCAT TCCTCATCAA | 540 |
| AGAAGATCAA TTGAATGCTG CACAGAAAGG AACGAATGTA ATAAAGACCT ACACCCTACA | 600 |
| CTGCCTCCAT TGAAAAACAG AGATTTTGTT GATGGACCTA TACACCACAG GGCTTTACTT | 660 |
| ATATCTGTGA CTGTCTGTAG TTTGCTCTTG GTCCTTATCA TATTATTTTG TTACTTCCGG | 720 |
| TATAAAAGAC AAGAAACCAG ACCTCGATAC AGCATTGGGT TAGAACAGGA TGAAACTTAC | 780 |
| ATTCCTCCTG GAGAATCCCT GAGAGACTTA ATTGAGCAGT CTCAGAGCTC AGGAAGTGGA | 840 |
| TCAGGCCTCC CTCTGCTOGT CCAAAGGACT ATAGCTAAGC AGATTCAGAT GGTGAAACAG | 900 |
| ATTGGAAAAG GTCGCTATGG GGAAGTTTGG ATGGGAAAGT GGCGTGGCGA AAAGGTAGCT | 960 |
| GTGAAAGTGT TCTFCACCAC AGAGGAAGCC AGCTGGTTCA GAGAGACAGA AATATATCAG | 1020 |
| ACAGTGTTGA TGAGGCATGA AAACATTTTG GGTTTCATTG CTGCAGATAT CAAAGGGACA | 1080 |
| GGGTCCTGGA CCCAGTTGTA CCTAATCACA GACTATCATG AAAATGGTTC CCTTTATGAT | 1140 |
| TATCTGAAGT CCACCACCCT AGACGCTAAA TCAATGCTGA AGTTAGCCTA CTCTTCTGTC | 1200 |
| AGTGGCTTAT GTCATTTACA CACAGAAATC TTTAGTACTC AAGGCAAACC AGCAATTGCC | 1260 |
| CATCGAGATC TGAAAAGTAA AAACATTCTG GTGAAGAAAA ATGGAACTTG CTGTATTGCT | 1320 |
| GACCTGGGCC TGGCTGTTAA ATTIATTAGT GATACAAATG AAGTTGACAT ACCACCTAAC | 1380 |
| ACTCGAGTTG GCACCAAACG CTATATGCCT CCAGAAGTGT TGGACGAGAG CTTGAACAGA | 1440 |
| AATCACTTCC AGTCTTACAT CATGGCTGAC ATGTATAGTT TTGGCCTCAT CCTTTGGGAG | 1500 |
| GTTGCTAGGA GATGTGTATC AGGAGGTATA GTGGAAGAAT ACCAGCTTCC TTATCATGAC | 1560 |
| CTAGTGCCCA GTGACCCCTC TTATOAGGAC ATGAGGGAGA TTGTGTGCAT CAAGAAGTTA | 1620 |
| CGCCCCTCAT TCCCAAACCG GTGGAGCAGT GATGAGTGTC TAAGGCAGAT GGGAAAACTC | 1680 |
| ATGACAGAAT GCTGGGCTCA CAATCCTGCA TCAAGGCTGA CAGCCCTGCG GGTTAAGAAA | 1740 |
| ACACTTGCCA AAATGTCAGA GTCCCAGGAC ATTAAACTCT GATAGGAGAG GAAAAGTAAG | 1800 |
| CATCTCTGCA GAAAGCCAAC AGGTACTCTT CTGTTTGTGG GCAGAGCAAA AGACATCAAA | 1860 |
| TAAGCATCCA CAGTACAAGC CTTGAACATC GTCCTGCTTC CCAGTGGGTT CAGACCTCAC | 1920 |
| CTTTCAGGGA GCGACCTGGG CAAAGACAGA GAAGCTCCCA GAAGGAGAGA TTGATCCGTG | 1980 |
| TCTGTTTGTA GGCGGAGAAA CCGTTGGGTA ACTTGTTCAA GATATGATGC AT | |

Seq ID NO: 6 Protein squence:
Protein Accession #: none found

| | |
|---|---|
| 1 11 21 31 41 51 | |
| \| \| \| \| \| \| | |
| MLLRSAGKLN VGTKKEDGES TAPTPRPKVL RCKCHHHCPE DSVNNICSTD GYCFTMIEED | 60 |
| DSGLPVVTSG CLGLEGSDFQ CRDTPIPHQR RSIECCTERN ECNKDLHPTL PPLKNRDFVD | 120 |
| GPIHHRALLI SVTVCSLLLV LIILECYFRY KRQETRPRYS IGLEQOETYI PPGESLRDLI | 180 |
| EQSQSSGSGS GLPLLVQRTI AXQIQMVKQI GKGRYGEVWM GKWRGEKVAV KVFFTTEEAS | 240 |
| WFRETEIYQT VLMRHENILG FIAADIKGTG SWTQLYLITD YHENGSLYDY LKSTTLDAKS | 300 |
| MLKLAYSSVS GLCHLHTEIF STQGKPAIAH RDLKSKNILV KKNGTCCIAD LGLAVKFISD | 360 |
| TNEVDIPPNT RVGTKRYMPP EVLDESLNRN HFQSYIMADM YSFGLILWEV ARRCVSGGIV | 420 |
| EEYQLPYHDL VPSDPSYEDM RETVCIKKLR PSFPNRWSSD ECLRQMGKLM TECWAHNPAS | 480 |
| RLTALRVKKT LAKMSESQDI KL | |

Seq ID NO: 7 DNA sequence
Nucleic Acid Accession #: none found
Coding sequence: 482-3007 (underlined sequences correspond to start
and stop codons)

| | |
|---|---|
| 1 11 21 31 41 51 | |
| \| \| \| \| \| \| | |
| AACTGAGCTA ACAAGAAATA CTAGAAAAGG AGGAAGGAGA ACATTGCTGC AGCTTGGATC | 60 |
| TACAACCTAA GAAAGCAAGA GTGATCAATC TCAGCTCTGT TAAACATCTT GTTTACTTAC | 120 |
| TGCATTCAGC AGCTTGCAAA TGOYfAACTA TATGCAAAAA AGTCAGCATA GCTGTGAAGT | 180 |
| ATGCCGTGAA TTTTAATTGA GGGAAAAAGG GACAATTGCT TCAGGATGCT CTAGTATGCA | 240 |
| CTCTGCTTIA AATATTTTCA ATGAAATGCT CAGTATTCTA TCTTTGACCA GAGGTTTTAA | 300 |
| CTTTATGAAG CTATGGGACT TGACAAAAAG TGATATTTGA GAAGAAAGTA CGCAGTGGTT | 360 |
| GGTGTTTTCT TTTTTTTAAT AAAGGAATTG AATTACTTTG AACACCTCTT CCAGCTGTGC | 420 |
| ATTACAGATA ACGTCAGGAA GAGTCTCTGC TTTACAGAAT CGGATTTCAT CACATGACAA | 480 |
| CATGAAGCTG TGGATTCATC TCTTTTATTC ATCTCTCCTT GCCTGTATAT CTTTACACTC | 540 |
| CCAAACTCCA GTGCTCTCAT CCAGAGGCTC TTGTGATTCT CTTTGCAATT GTGAGGAAAA | 600 |
| AGATGGCACA ATGCTAATAA ATTGTGAAGC AAAAGGTATC AAGATGGTAT CTOAAATAAG | 660 |
| TGTGCCACCA TCACGACCTT TCCAACTAAG CTTATTAAAT AACGGCTTGA CGATGCTTCA | 720 |
| CACAAATGAC TTTTCTGGGC TTACCAATGC TATTTCAATA CACCTTGGAT TTAACAATAT | 780 |
| TGCAGATATT GAGATAGGTG CATTTAATGG CCTTGGCCTC CTGAAACAAC TTCATATCAA | 840 |
| TCACAATTCT TTAGAAATTC TTAAAGAGGA TACTTTCCAT GGACTOGAAA ACCTGGAATT | 900 |
| CCTGCAAGCA GATAACAATT TTATCACAGT GATTGAACCA AGTGCCTTTA GCAAGCTCAA | 960 |
| CAGACTCAAA GTGTTAATTT TAAATGACAA TGCTATTGAG AGTCTTCCTC CAAACATCTT | 1020 |
| CCGATTTGTT CCTTTAACCC ATCTAGATCT TCGTGGAAAT CAATTACAAA CATTGCCTTA | 1080 |
| TGTTGGTTTT CTCGAACACA TTGGCCGAAT ATTGGATCTT CAGTTGGAGG ACAACAAATG | 1140 |
| GGCCTGCAAT TGTGACTTAT TGCAGTTAAA AACTTGGTTG GAGAACATGC CTCCACAGTC | 1200 |
| TATAATTGGT GATGTTGTCT GCAACAGCCC TCCATTTTTT AAACGAAGTA TACTCAGTAG | 1260 |
| ACTAAAGAAG GAATCTATTT GCCCTACTCC ACCAGTGTAT GAAGAACATG AGGATCCTTC | 1320 |
| AGGATCATTA CATCTGGCAG CAACATCTTC AATAAATGAT AGTCGCATGT CAACTAAGAC | 1380 |
| CACGTCCATT CTAAAACTAC CCACCAAAGC ACCAGGTTTG ATACCTTATA TTACAAAGCC | 1440 |
| ATCCACTCAA CTTCCAGGAC C3TACTGCCC TATTCCTTGT AACTGCAAAG TCCTATCCCC | 1500 |
| ATCAGGACTT CTAATACATT GTCAGGAGCG CAACATTGAA AGCTTATCAG ATCTGAGACC | 1560 |
| TCCTCCGCAA AATCCTAGAA AGCTCATTCT AGCGGGAAAT ATTATTCACA GTTTAATGAA | 1620 |
| GTCTGATCTA GTGGAATATT TCACTTTGGA AATGCTTCAC TTGGGAAACA ATCGTATTGA | 1680 |
| AGTTCTTGAA GAAGGATCGT TTATGAACCT AACGAGATTA CAAAAACTCT ATCTAAATGG | 1740 |
| TAACCACCTG ACCAAATTAA GTAAAGGCAT GTTCCTTGGT CTCCATAATC TTGAATACTT | 1800 |
| ATATCTTGAA TACAATGCCA TTAAGGAAAT ACTGCCAGGA ACCTTTAATC CAATGCCTAA | 1860 |
| ACTTAAAGTC CTGTATTTAA ATAACAACCT CCTCCAAGTT TTACCACCAC ATATTTTTTC | 1920 |
| AGGGGTTCCT CTAACTAAGG TAAATCTTAA AACAAACCAG TTTACCCATC TACCTGTAAG | 1980 |
| TAATATTTTG GATGATCTTG ATTTACTAAC CCAGATTGAC CTTGAGGATA ACCCCTGGGA | 2040 |
| CTGCTCCTGT GACCTGGTTG GACTGCAGCA ATGGATACAA AAGTTAAGCA AGAACACAGT | 2100 |
| GACAGATOAC ATCCTCTGCA CTTCCCCCGG GCATCTCGAC AAAAAGGAAT TGAAAGCCCT | 2160 |
| AAATAGTGAA ATTCTCTGTC CAGGTTTAGT AAATAACCCA TCCATGCCAA CACAGACTAG | 2220 |
| TTACCTTATG GTCACCACTC CTGCAACAAC AACAAATACG GCTGATACTA TTTTACGATC | 2280 |
| TCTTACGGAC GCTGTGCCAC TGTCTGTTCT AATATTGGGA CTTCTGATTA TGTTCATCAC | 2340 |
| TATTGTTTTC TGTGCTGCAG GGATAGTGGT TCTTGTTCTT CACCGCAGGA GAAGATACAA | 2400 |
| AAAGAAACAA GTAGATGAGC AAATGAGAGA CAACAGTCCT GTGCATCTTC AGTACAGCAT | 2460 |
| GTATGGCCAT AAAACCACTC ATCACACTAC TGAAAGACCC TCTGCCTCAC TCTATGAACA | 2520 |
| GCACATGGTG AGCCCCATGG TTCATGTCTA TAGAAGTCCA TCCTTTGGTC CAAAGCATCT | 2580 |
| GGAAGAGGAA GAAGAGAGGA ATGAGAAAGA AGGAAGTGAT GCAAAACATC TCCAAAGAAG | 2640 |
| TCTITfGGAA CAGGAAAATC ATTCACCACT CACAGGGTCA AATATGAAAT ACAAAACCAC | 2700 |
| GAACCAATCA ACAGAATTTT TATCCTTCCA AGATGCCAGC TCATTGTACA GAAACATTTT | 2760 |
| AGAAAAAGAA AGGGAACTTC AGCAACTGGG AATCACAGAA TACCTAAGGA AAAACATTGC | 2820 |
| TCAGCTCCAG CCTGATATGG AGGCACATTA TCCTGGAGCC CACGAAGAGC TGAAGTTAAT | 2880 |
| GGAAACATTA ATGTACTCAC GTCCAAGGAA GGTATTAGTG GAACAGACAA AAAATGAGTA | 2940 |
| TTTTGAACTT AAAGCTAATf TACATGCTGA ACCTGACTAT TTAGAAGTCC TGGAGCAGCA | 3000 |
| AACATAGATG GAGAGT1TGA GGGCTTTCGC AGAAATGCTG TGATTCTGTT TTAAGTCCAT | 3060 |
| ACCGGGTAAA TAAGTGCCTT ACGTGAGTGT GTCATCAATC AGAACCTAAG CACAGCAGTA | 3120 |
| AACTATGGGG AAAAAAAAAG AAGAAGAAAA GAAACTCAGG GATCACTGGG AGAAGCCATG | 3180 |
| GCATTATCTT CAGGCAATTT AGTCTGTCCC AAATAAAATC AATCCTTGCA TGTAAATC | |

Seq ID NO: 8 Protein sequence:
Protein Accession #: none found

| | |
|---|---|
| 1 11 21 31 41 51 | |
| \| \| \| \| \| \| | |
| MKLWIHLFYS SLLACISLHS QTPVLSSRGS CDSLCNCEEK DGTMLINCEA KGIKMVSEIS | 60 |
| VPPSRPFQLS LLNNGLTMLH TNDFSGLTNA ISIHLGFNNI ADIEIGAENG LGLLKQLHIN | 120 |
| HNSLEILKED TFHGLENLEF LQADNNEITV IEPSAFSKLN RLKVLILNDN AIESLPPNIF | 180 |
| RFVPLTHLDL RGNQLQTLPY VGFLEHIGRI LDLQLEDNKW ACNCDLLQLK TWLENMPPQS | 240 |
| IIGDVVCNSP PFFKGSILSR LKKESICPTP PVYEEHEDPS GSLHLAATSS INDSRMSTKT | 300 |
| TSILKLPTKA PGLIPYITKP STQLPGPYCP IPCNCKVLSP SGLLHICQER NIESLSDLRP | 360 |
| PPQNPRKLIL AGNIIHSLMK SDLVEYFTLE MLHLGNNRIE VLEEGSFMNL TRLQKLYLNG | 420 |
| NHLTKLSKGM FLGLHNLEYL YLEYNAIKEI LPGTFNFMPK LKVLYLNNNL LQVLPPIIFS | 480 |
| GVPLTKVNLK TNQFTHLPVS NILDDLDLLT QIDLEDNPWD CSCDLVOLQQ WIQKLSKNTV | 540 |
| TDDILCTSPG HLDKKELKAL NSEILCPGLV NNPSMPTQTS YLMVTTPATT TNTADTILRS | 600 |
| LTDAVPLSVL ILGLLIMFIT IVFCAAGIVV LVLHRRRRYK KKQVDEQMRD NSPVHLQYSM | 660 |
| YGHKTTHHTT ERPSASLYEQ HMVSPMVHVY RSPSFGPKHL EEEEERNEKE GSDAKHLQRS | 720 |
| LLEQENHSPL TGSNMKYKTT NQSTEFLSFQ DASSLYRNIL EKERELQQLG ITEYLRXNIA | 780 |
| QLQPDMEAHY PGAHEELKLM ETLMYSRPRK VLVEQTKNEY FELKANLHAE PDYLEVLEQQ | 840 |
| T | |

Seq ID NO: 9 DNA sequence
Nucleic Acid Accession #: NM_003474
Coding sequence: 307-3036 (underlined sequences cnrrespnnd to start
and stop codons)

| | |
|---|---|
| 1 11 21 31 41 51 | |
| \| \| \| \| \| \| | |
| CACTAACGCT CTTCCTAGTC CCCGGGCCAA CTCGOACAGT TTGCTCATTT ATTGCAACGG | 60 |
| TCAAGGCTGG CTTGTGCCAG AACGGCGCGC GCGCGACGCA CGCACACACA CGGGGGGAAA | 120 |
| CTTTTTTAAA AATGAAAGGC TAGAAGAGCT CAGCGGCGGC GCGGGCCGTG CGCGAGGGCT | 180 |
| CCGGAGCTGA CTCGCCGAGG CAGGAAATCC CTCCGGTCGC GACGCCCGGC CCCGCTCGGC | 240 |
| GCCCGCGTGG GATGGTGCAG CGCTCGCCGC CGGGCCCGAG AGCTGCTGCA CTGAAGGCCG | 300 |
| GCGACGATGG CAGCGCGCCC GCTGCCCGTG TCCCCCGCCC GCGCCCTCCT GCTCGCCCTG | 360 |
| GCCGGTGCTC TGCTCGCGCC CTGCGAGGCC CGAGGGGTGA GCTTATGGAA CGAAGGAAGA | 420 |
| GCTGATGAAG TTGTCAGTOC CTCTGTTCGG AGTGGGGACC TCTGGATCCC AOTGAAGAGC | 480 |
| TTCGACTCCA AGAATCATCC AGAAGTGCTG AATATTCGAC TACAACGGGA AAGCAAAGAA | 540 |
| CTGATCATAA ATCTGGAAAG AAATGAAGGT CTCATGGCCA GCAGTTTCAC GGAAACCCAC | 600 |
| TATCTGCAAG ACGGTACTOA TGTCTCCCTC GCTCGAAATT ACACGGTAAT TCTGGGTCAC | 660 |
| TGTTACTACC ATGGACATGT ACGGGGATAT TCTGATTCAG CAGTCAGTCT CAGCACGTGT | 720 |
| TCTGGTCTCA GGGGACTTAT TGTGTTTGAA AATGAAAGCT ATGTCTTAGA ACCAATGAAA | 780 |
| AGTGCAACCA ACAGATACAA ACTCTTCCCA GCGAAGAAGC TGAAAAGCGT CCGGGGATCA | 840 |
| TGTGGATCAC ATCACAACAC ACCAAACCTC GCTGCAAAGA ATGTGTTTCC ACCACCCTCT | 900 |
| CAGACATGGG CAAGAAGGCA TAAAAGAGAG ACCCTCAAGG CAACTAAGTA TGTGGAGCTG | 960 |
| GTGATCGTGG CAGACAACCG AGAGTTTCAG AGGCAAGGAA AAGATCTGGA AAAAGTTAAG | 1020 |
| CAGCGATTAA TAGAGATTGC TAATCACGTT GACAAGTTTT ACAGACCACT GAACATTCGG | 1080 |
| ATCGTGTTGG TAGGCGTGGA AGTGTGGAAT GACATGGACA AATGCTCTGT AAGTCAGGAC | 1140 |
| CCATGCACCA GCCTCCATGA AGGTCTGGAC TGGAGGAAGA TGAAGCTTCT ACCTCGCAAA | 1200 |
| TCCCATGACA ATGCGCAGCT TGTCAGTGGG GTTTATTTCC AAGGGACCAC CATCGGCATG | 1260 |
| GCCCCAATCA TGAGCATGTG CACGGCAGAC CAGTCTGGGG GAATTGTCAT GGACCATTCA | 1320 |
| GACAATCCCC TTGGTGCAGC CGTGACCCTG GCACATGAGC TGGGCCACAA TTTCGGGATG | 1380 |
| AATCATGACA CACTOGACAG GGGCTGTAGC TGTCAAATGG CGGTTGAGAA AGGAGGCTGC | 1440 |
| ATCATGAACG CTTCCACCGG GTACCCATTT CCCATGGTGT TCAGCAGTTG CAGCAGGAAG | 1500 |
| GACTTGGAGA CCAGCCTGGA GAAAGGAATG GGGGTGTGCC TGTTTAACCT GCCGGAAGTC | 1560 |
| AGGGAGTCTT TCGGGGGCCA GAAGTGTGGG AACAGATTTG TGGAAGAAGG AGAGGAGTGT | 1620 |
| GACTGTGGGG AGCCAGAGGA ATGTATGAAT CGCTGCTGCA ATGCCACCAC CTGTACCCTG | 1680 |
| AAGCCGGACG CTGTGTGCGC ACATGGGCTG TGCTGTGAAG ACTGCCAGCT GAAGCCTGCA | 1740 |
| GGAACAGCGT GCAGGGACTC CAGCAACTCC TGTGACCTCC CAGAGTTCTG CACAGGGGCC | 1800 |
| AGCCCTCACT GCCCAGCCAA CGTGTACCTG CACGATGGGC ACTCATGTCA GGATGTGGAC | 1860 |
| GOCTACTOCT ACAATGGCAT CTGCCAGACT CACGAGCAGC AGTGTGTCAC ACTCTGGGGA | 1920 |
| CCAGGTGCTA AACCTGCCCC TGGGATCTGC TTTGAGAGAG TCAATTCTGC AGGTGATCCT | 1980 |
| TATGGCAACT TTGGCAAAGT CTCGAAGAGT TCCTTTGCCA AATGCGAGAT GAGAGATGCT | 2040 |
| AAATGTGGAA AAATCCAGTG TCAAGGAGGT GCCAGCCGGC CAGTCATTGG TACCAATGCC | 2100 |
| GTTTCCATAG AAACAAACAT CCCCCTGCAG CAAGGAGOCC GGATTCTGTG CCGGGGGACC | 2160 |
| CACGTGTACT TGGGCGATGA CATGCCGGAC CCAGGGCTTG TGCTTGCAGG CACAAAGTGT | 2220 |
| GCAGATGGAA AAATCTGCCT GAATCGTCAA TGTCAAAATA TTAGTGTCTT TGGGGTTCAC | 2280 |
| GAGTGTGCAA TGCAGTGCCA CGGCAGAGGG GTGTGCAACA ACAGGAAGAA CTGCCACTGC | 2340 |
| GAGGCCCACT GGGCACCTCC CTTCTGTGAC AAGTTTGGCT TTGGAGGAAG CACAGACAGC | 2400 |
| GGCCCCATCC GGCAAGCAGA TAACCAAGGT TTAACCATAG GAATTCTGGT GACCATCCTG | 2460 |
| TGTCTTCTTG CTGCCGGATT TGTGGTTTAT CTCAAAAGGA AGACCTTGAT ACGACTGCTG | 2520 |
| TTTACAAATA AGAAGACCAC CATTGAAAAA CTAAGGTGTG TGCGCCCTTC CCGGCCACCC | 2580 |
| CGTGGCTTCC AACCCTGTCA GGCTCACCTC GGCCACCTTG GAAAAGGCCT GATGAGGAAG | 2640 |
| CCGCCAGATT CCTACCCACC GAAGGACAAT CCCAGGAGAT TGCTGCAGTG TCAGAATGTT | 2700 |
| GACATCAGCA GACCCCTCAA CGGCCTGAAT GTCCCTCAGC CCCAGTCAAC TCAGCGAGTG | 2760 |
| CTTCCTCCCC TCCACCGGGC CCCACGTGCA CCTAGCGTCC CTGCCAGACC CCTGCCAGCC | 2820 |
| AAGCCTGCAC TTAGGCAGGC CCAGGGOACC TGTAAGCCAA ACCCCCCTCA GAAGCCTCTG | 2880 |
| CCTGCAGATC CTCTGGCCAG AACAACTCGG CTCACTCATG CCTfGGCCAG GACCCCAGGA | 2940 |
| CAATGGGAGA CTGGGCTCCG CCTGGCACCC CTCAGACCTG CTCCACAATA TCCACACCAA | 3000 |
| GTGCCCAGAT CCACCCACAC CGCCTATATT AAGTGAGAAG CCGACACCTT TTTTCAACAG | 3060 |
| TGAAGACAGA AGTTTGCACT ATCTTTCAGC TCCAGTTGGA GTTTTTTGTA CCAACTTTTA | 3120 |
| GGATTTTTTT TAATGTTTAA AACATCATTA CTATAAGAAC CTATAAGAAC TTTGAGCTAC | 3180 |
| GCTGTGCTGT GCTATGGTGC TCTGTCTACT TOCACAGOTA CTTGTAAATT ATTAATTTAT | 3240 |
| GCAGAATGTT GATTACAGTG CAGTGCGCTG TAGTAGGCAT TTTTACCATC ACTGAGTTTT | 3300 |
| CCATGGCAGG AAGGCTTGTT GTGCTTTTAG TATTTTAGTG AACTTGAAAT ATCCTGCTTG | 3360 |
| ATGGGATTCT GGACAGGATG TGTTTGCTTT CTGATCAAGG CCTTATTGGA AAGCAGTCCC | 3420 |
| CCAACTACCC CCAGCTGTGC TTATGGTACC AGATGCAGCT CAAGAGATCC CAAGTAGAAT | 3480 |
| CTCAGTTGAT TTTCTGGATT CCCCATCTCA GGCCAGAGCC AAGGGGCTTC AGGTCCAGGC | 3540 |
| TGTGTTTGGC TTTCAGGGAG GCCCTGTGCC CCTTGACAAC TGGCAGGCAG GCTCCCAGGG | 3600 |
| ACACCTGGGA GAAATCTGGC TTCTGGCCAG GAAGCTTTGG TGAGAACCTG GGTTGCAGAC | 3660 |
| AGGAATCTTA AGGTGTAGCC ACACCAGGAT AOAGACTGGA ACACTAGACA AGCCAGAACT | 3720 |
| TGACCCTGAG CTGACCAGCC GTGAGCATGT TTGGAAGGGG TCTGTAGTGT CACTCAAGGC | 3780 |
| GGTGCTTGAT AGAAATGCCA AGCACTTCTT TTTCTCGCTG TCCTTTCTAG AGCACTGCCA | 3840 |
| CCAGTAGGTT ATTTTAGCTG GGAAAGGTGG TGTTTCTGTA AGAAACCTAC TGCCCAGGCA | 3900 |
| CTGCAAACCG CCACCTCCCT ATACTGCTTG GAGCTGAGCA AATCACCACA AACTGTAATA | 3960 |
| CAATGATCCT GTATTCAGAC AGATGAGGAC TTTCCATGGG ACCACAACTA TTTTCAGATG | 4020 |
| TGAACCATTA ACCAGATCTA GTCAATCAAG TCTGTTTACT GCAAGGTTCA ACTTATTAAC | 4080 |
| AATTAGGCAG ACTCTTTATG CTTGCAAAAA CTACAACCAA TGGAATGTGA TGTTCATGGG | 4140 |
| TATAGTTCAT GTCTGCTATC ATTATTCGTA GATATTOGAC AAAGAACCTT CTCTATGGGG | 4200 |
| CATCCTCTTT TTCCAACTTG GCTGCAGGAA TCTTTAAAAG ATGCTTTTAA CAGAGTCTGA | 4260 |
| ACCTATTTCT TAAACACTTG CAACCTACCT GTTGAGCATC ACAGAATGTG ATAAGGAAAT | 4320 |
| CAACTTGCTT ATCAACTTCC TAAATATTAT GAGATGTGGC TTGGGCAGCA TCCCCTTGAA | 4380 |
| CTCTTCACTC TTCAAATGCC TGACTAGGGA GCCATGTTTC ACAAGGTCTT TAAAGTGACT | 4440 |
| AATGGCATGA GAAATACAAA AATACTCAGA TAAGGTAAAA TGCCATGATG CCTCTGTCTT | 4500 |
| CTGGACTGGT TTTCACATTA GAAGACAATT GACAACAGTT ACATAATTCA CTCTGAGTGT | 4560 |
| TTTATGAGAA AGCCTTCTTT TGGGGTCAAC AGTTTTCCTA TGCTTTGAAA CAGAAAAATA | 4620 |
| TGTACCAAGA ATCTTGGTTT GCCYTCCAGA AAACAAAACT GCATTTCACT TTCCCGGTGT | 4680 |
| TCCCCACTGT ATCTAGGCAA CATAGTATTC ATGACTATGG ATAAACTAAA CACGTGACAC | 4740 |
| AAACACACAC AAAAGGGAAC CCAGCTCTAA TACATTCCAA CTCGTATAGC ATGCATCTGT | 4800 |
| TTATTCTATA GTTATTAAGT TCTTTAAAAT GTAAAGCCAT GCTGGAAAAT AATACTGCTG | 4860 |
| AGATACATAC AGAATTACTG TAACTGATTA CACTTGGTAA TTGTACTAAA GCCAAACATA | 4920 |
| TATATACTAT TAAAAAGGTT TACAGAATTT TATGGTGCAT TACGTGGGCA TTGTCTTTTT | 4980 |
| AGATGCCCAA ATCCTTAGAT CTGGCATGTT AGCCCTTCCT CCAATTATAA GAGGATATGA | 5040 |
| ACCAAAAAAA AAAAAAAAAA AA | |

Seq ID NO: 10 Protein sequence:
Protein Accession #: NP_003465.2

| | |
|---|---|
| 1 11 21 31 41 51 | |
| \| \| \| \| \| \| | |
| MAARPLPVSP ARALLLALAG ALLAPCEARG VSLWNEGRAD EVVSASVRSG DLWIPVKSFD | 60 |
| SKNHPEVLNI RLQRESKELI INLERNEGLI ASSFTETHYL QDGTDVSLAR NYTVILGHCY | 120 |
| YHGHVRGYSD SAVSLSTCSG LRGLIVFENE SYVLEPMKSA TNRYKLFPAK KLKSVRGSCG | 180 |
| SHHNTPNLAA KNVFPPPSQT WARRHKRETL KATKYVELVI VADNREFQRQ GKDLEKVKQR | 240 |
| LIEIANHVDK FYRPLNIRIV LVGVEVWNDM DKCSVSQDPF TSLHEFLDWR KMKLLPRKSH | 300 |
| DNAQLVSGVY FQGTTIGMAP IMSMCTADQS GGIVMDHSDN PLGAAVTLAH ELGHNFGMNH | 360 |
| DTLDRGCSCQ MAVEKGGCIM NASTGYPFPM VFSSCSRKDL ETSLEKGMGV CLFNLPEVRE | 420 |
| SFGGQKCGNR FVEEGEECDC GEPEECMNRC CNATTCTLKP DAVCAHGLCC EDCQLKPAGT | 480 |
| ACRDSSNSCD LPEFCTGASP HCPANVYLHD GHSCQDVDGY CYNGICQTHE QQCVTLWGPG | 540 |
| AKPAPGICFE RVNSAGDPYG NCGKVSKSSF AKCEMRDAKC GKIQCQGGAS RPVIGTNAVS | 600 |
| IETNIPLQQG GRILCRGTHV YLGDDMPDPG LVLAGTKCAD GKICLNRQCQ NISVFGVHEC | 660 |
| AMQCHGRGVC NNRKNCHCEA HWAPPFCDKF GFGGSTDSGP IRQADNQGLT IGILVTILCL | 720 |
| LAAGFVVYLK RKTLIRLLFT NKKTTIEKLR CVRPSRPPRG FQPCQAHLGH LGKGLMRKPP | 780 |
| DSYPPKDNPR RLLQCQNVDI SRPLNGLNVP QPQSTQRVLP PLHRAPRAPS VPARPLPAKP | 840 |
| ALRQAQGTCK PNPPQKPLPA DPLARTTRLT HALARTPGQW ETGLRLAPLR PAPQYPHQVP | 900 |
| RSTHTAYIK | |

Seq ID NO: 11 DNA sequeNucleic Acid Accession #: NM_001394
Coding sequence: 400-1584 (underlined seqaences correspond to start
and slop codons)

| | |
|---|---|
| 1 11 21 31 41 51 | |
| \| \| \| \| \| \| | |
| GGCACGAGGG CTGAGCGCCG GAGGAGCGTA GGCAGGGCAG CGCTGGCGCC AGCGGCOACA | 60 |
| GGAGCCGCGC GACCGGCAAA AATACACGGG AGGCCGTCGC CGAAAAGAGT CCGCGGTCCT | 120 |
| CTCTCGTAAA CACACTCTCC TCCACCGGCG CCTCCCCCTC CGCTCTGCGC GCCGCCCGGC | 180 |
| TGGGCGCCCG AGGCCGCTCC GACTGCTATG TGACCGCGAG GCTGCGGGAG GAAGGGGACA | 240 |
| GGGAAGAAGA GGCTCTCCCG CGGGAGCCCT TGAGGACCAA GTTTGCGGCC ACTTCTGCAG | 300 |
| GCGTCCCTTC TTAGCTCTCG CCTGCCCCTT TCTGCAGCCT AGGCGGCCCA GGTTCTCTTC | 360 |
| TCTTCCTCGC GCGCCCAGCC GCCTCGGTTC CCGGCGACCA TGGTGACGAT GGAGGAGCTG | 420 |
| CGGGAGATGG ACTGCAGTGT GCTCAAAAGG CTGATGAACC GGGACGAGAA TGGCGGCGGC | 480 |
| GCGGGCGGCA GCGGCAGCCA CGGCACCCTG GGGCTGCCGA GCGGCGGCAA GTGCCTGCTG | 540 |
| CTGGACTGCA GACCGTTCCT GGCGCACAGC GCGGGCTACA TCCTAGGTTC GGTCAACGTG | 600 |
| CGCTGTAACA CCATCGTGCG GCGGCGGGCT AAGGGCTCCG TGAGCCTGGA GCAGATCCTG | 660 |
| CCCGCCGAGG AGGAGGTACG CGCCCGCTTG CGCTCCGGCC TCTACTCGGC GGTCATCGTC | 720 |
| TACGACGAGC GCAGCCCGCG CGCCGAGAGC CTCCGCGAGG ACAGCACCGT GTCGCTGGTG | 780 |
| GTGCAGGCGC TGCGCCGCAA CGCCGAGCGC ACCGACATCT GCCTGCTCAA AGGCGGCTAT | 840 |
| GAGAGGTTTT CCTCCGAGTA CCCAOAATGC TGTTCTAAAA CCAAGGCCCT GGCAGCCATC | 900 |
| CCACCCCCGG TTCCCCCCAG COCCACAGAG CCCTTGGACC TGGGCTGCAG CTCCTGTGGG | 960 |
| ACCCCACTAC ACGACCAGGG GGGTCCTGTG GAGATCCTTC CCTTCCTCTA CCTCGGCAGT | 1020 |
| GCCTACCATG CTGCCCGGAG AGACATOCTO GACGCCCTGG GCATCACGGC TCTGTTGAAT | 1080 |
| GTCTCCTCGG ACTGCCCAAA CCACTTTGAA GGACACTATC AGTACAAGTG CATCCCAGTG | 1140 |
| GAAGATAACC ACAAGGCCGA CATCAGCTCC TGGTtCATGG AAGCCATAGA GTACATCGAT | 1200 |
| GCCGTGAAGG ACTGCCGTGG GCGCGTGCTG GTGCACTGCC AGGCGGGCAT CTCGCGGTCG | 1260 |
| GCCACCATCT GCCTGGCCTA CCTGATGATG AAGAAACGGG TGAGGCTGGA GGAGGCCTTC | 1320 |
| GAGTTCGTTA AGCAGCGCCG CAGCATCATC TCGCCCAACT TCAGCTTCAT GGGGCAGCTG | 1380 |
| CTGCAGTTCG AGTCCCAGGT GCTGGCCACG TCCTGTGCTG CGGAGGCTGC TAGCCCCTCG | 1440 |
| OGACCCCTGC GGGAGCGGGG CAAGACCCCC GCCACCCCCA CCTCGCAGTT CGTCTTCAGC | 1500 |
| TfTCCGGTCT CCGTGGGCGT OCACTCGGCC CCCAGCAGCC TGCCCTACCT GCACAGCCCC | 1560 |
| ATCACCACCT CTCCCAGCTG TTAGAGCCGC CCTGGGGGCC CCAGAACCAG AGCTGGCTCC | 1620 |
| CAGCAAGGGT AGGACGGGCC GCATGCGGCA GAAAGTTGGG ACTGAGCAGC TGGGAGCAGG | 1680 |
| CGACCGAGCT CCTTCCCCAT CATTTCTCCT TGGCCAACGA CGAGGCCAGC CAGAATGGCA | 1740 |
| ATAAGGACTC CGAATACATA ATAAAAGCAA ACAGAACACT CCAACTTAGA GCAATAACCG | 1800 |
| GTGCCGCAGC AGCCAGGGAA GACCTTGGTT TGGTTTATGT GTCAGTTTCA CTTTTCCGAT | 1860 |
| AGAAATTTCT TACCTCATTT TTTTAAGCAG TAAGGCTTGA AGTGATOAAA CCCACAGATC | 1920 |
| CTAGCAAATG TGCCCAACCA GTTTTACTAA AGGGGGAGGA AGGGAGGGCA AAGGGATGAG | 1980 |
| AAGACAAGTT TCCCAGAAGT GCCTGGTTCT GGGTACTfGT CCCTTTGTTG TCGTTGTTGT | 2040 |
| AGTTAAAGGA ATTTCATTTT TAAAAGAAAT CTTCGAAGGT GTGGTTTTCA TTTCTCAGTC | 2100 |
| ACCAACAGAT GAATAATTAT GCTTAATAAT AAAGTATTTA TTAAGACTTT CTTCAOAGTA | 2160 |
| TGAAAGTACA AAAAGTCTAG TTACAGTGGA TTTAGAATAT ATTTATGTTG ATGTCAAACA | 2220 |
| GCTGAGCACC GTAGCATGCA GATGTCAAGG CAGTTAGGAA GTAAATGGTO TCTTGTAGAT | 2280 |
| ATGTGCAAGG TAGCATGATG AGCAACTTGA GTTTGTTGCC ACTGAGAAGC AGOCGGGTTG | 2340 |
| GGTGGGAGGA GGAAGAAAOG GAAGAATTAG GTTTGAATTG CTTTTTAAAA AAAAAAGAAA | 2400 |
| AGAAAAAGAC AGCATCTCAC TATGTTGCCA AGGCTCATCT TGAGAAGCAG GCGGGTTGGG | 2460 |
| TGGGAGGAGG AAGAAAGGGA AOAATTAGGT TTGAATTGCT TTTTTAAAAA AAAA | |

Seq ID NO: 12 Protein sequence:
Protein Accession #: NP_001385

| | |
|---|---|
| 1 11 21 31 41 51 | |
| \| \| \| \| \| \| | |
| MVTMEELREM DCSVLKRLMN RDENGGGAGG SGSHGTLGLP SGGKCLLLDC RPFLAHSAGY | 60 |
| ILGSVNVRCN TIVRRRAKGS VSLEQILPAE EEVRARLRSG LYSAVIVYDE RSPRAESLRE | 120 |
| DSTVSLVVQA LRRNAERTDI CLLKGGYERF SSEYPEFCSK TKALAAIPPP VFPSATEPLD | 180 |
| LGCSSCGTPL HDQGGPVEIL PFLYLGSAYH AARRDMLDAL GITALLNVSS DCPNIWEGHY | 240 |
| QYKCIPVEDN HKADISSWFM EAIEYIDAVK DCRGRVLVHC QAGISRSATI CLAYLMMKKR | 300 |
| VRLEEAFEFV KQRRSIISPN FSFMGQLLQF ESQVLATSCA AEAASPSGPL RERGKTPATP | 360 |
| TSQFVFSFPV SVGVHSAPSS LPYLHSPITT SPSC | |

Seq ID NO: 13 DNA sequeNucleic Acid Accession #: none found
Coding sequence: 68-340 (underlined sequences correspond to start and
stop codons)

| | |
|---|---|
| 1 11 21 31 41 51 | |
| \| \| \| \| \| \| | |
| AGCGCCTTGC CTTCTCTTAG GCTTTGAAGC ATTTTTGTCT GTGCTCCCTG ATCTTCAGGT | 60 |
| CACCACCATG AAGTTCTTAG CAGTCCTGGT ACTCTTGGGA GTTTCCATCT TTCTGGTCTC | 120 |
| TGCCCAGAAT CCGACAACAG CTGCTCCAGC TGACACGTAT CCAGCTACTG GTCCTGCTGA | 180 |
| TGATGAAGCC CCTGATGCTG AAACCACTGC TGCTGCAACC ACTGCGACCA CTGCTGCTCC | 240 |
| TACCACTGCA ACCACCGCTG CTTCTACCAC TTCTCGTAAA GACATTCCAG TTTTACCCAA | 300 |
| ATGGGTTGGG GATCTCCCGA ATGGTAGAGT GTGTCCCTGA GATGGAATCA GCTTGAGTCT | 360 |
| TCTGCAATTG GGTCACAACT ATTCATGCTT CCTGTGATTT CATCCAACTA CTTACCTTGC | 420 |
| CTACGATATC CCCTTTATCT CTAATCAGTT TAITTTCTTT CAAATAAAAA ATAACTATGA | 480 |
| GCGAGCTAAC AT | |

Seq ID NO: 14 Protein sequence:
Protein Accession #: none found

| | |
|---|---|
| 1 11 21 31 41 51 | |
| \| \| \| \| \| \| | |
| MKFLAVLVLL GVSIFLVSAQ NPTTAAPADT YPATGPADDE APDAETTAAA TTATTAAPTT | 60 |
| ATTAASITAR KDIPVLPKWV GDLPNORVCP | |

Seq ID NO: 15 DNA sequeNucleic Acid Accession #: NM_016640.2
Coding sequence: 39-1358 (underlined sequences correspond to start and
stop codons)

| | |
|---|---|
| 1 11 21 31 41 51 | |
| \| \| \| \| \| \| | |
| GGTTAAGTTG ACCTCTGGGT CCGGAATCGC GGGCAAAGAT GGCGCCGGCC AGGTGTTGGA | 60 |
| GGCCTTTGCT ACGCGGTCCG AGGCTTTCAT TGCACACCGC GGCTAATGCC GCCGCCACGG | 120 |
| CTACAGAAAC GACCTCCCAA GACGTCGCGG CGACCCCCGT CGCGCGGTAC CCGCCGATTG | 180 |
| TGGCCTCCAT GACAGCCGAC AGCAAAGCTG CACGGCTGCG GCGGATCGAG CGCTGGCAGG | 240 |
| CGACGGTGCA CGCTGCGGAG TCGGTAGACG AGAAGCTGCG AATCCTCACC AAGATGCAGT | 300 |
| TTATGAAGTA CATGGTTTAC CCGCAGACCT TCGCACTGAA TGCCGACCGC TGGTACCAGT | 360 |
| ACTTCACCAA GACCGTGTTC CTGTCGGGTC TGCCGCCGCC CCCAGCGGAG CCCGAGCCCG | 420 |
| AGCCCGAACC CGAACCTGAA CCTGCGCTGG ACCTCGCGGC GCTGCGTGCG GTCGCCTGCG | 480 |
| ACTGCCTGCT GCAGGAGCAC TTCTACCTGC GGCGCAGGCG GCGCGTGCAC CGTTACGAGG | 540 |
| AGAGCGAGGT CATATCTTTG CCCTTCCTGG ATCAGCTGGT GTCAACCCTC GTGGGCCTCC | 600 |
| TCAGCCCACA CAACCCGGCC CTGGCCGCTG CCGCCCTCGA TTATAGATGC CCAGTTCATT | 660 |
| TTTACTGGGT GCGTGGTGAA GAAATTATTC CTCGTGGTCA TCGAAGAGGT CGAATTGATG | 720 |
| ACTTGCGATA CCAGATAGAT GATAAACCAA ACAACCAGAT TCGAATATCC AAGCAACTCG | 780 |
| CAGAGTTTGT GCCATTGGAT TATTCTGTTC CTATAGAAAT CCCCACTATA AAATGTAAAC | 840 |
| CAGACAAACT TCCATTATTC AAACGGCAGT ATGAAAACCA CATATTTGTT GGCTCAAAAA | 900 |
| CTGCAGATCC TTGCTGTTAC GGTCACACCC AGTTTCATCT GTTACCTGAC AAATTAAGAA | 960 |
| GGGAAAGGCT TTTGAGACAA AACTGTGCTG ATCAGATAGA AGTTGTTTTT AGAGCTAATG | 1020 |
| CTATTGCAAG CCTTTTTGCT TGGACTGGAG CACAAGCTAT GTATCAAGGA TTCTGGAGTG | 1080 |
| AAGCAGATGT TACTCGACCT TTTGTCTCCC AGGCTGTGAT CACAGATGGA AAATACTTTT | 1140 |
| CCTTTTTCTG CTACCAGCTA AATACTTTGG CACTGACTAC ACAAGCTGAT CAAAATAACC | 1200 |
| CTCGTAAAAA TATATGTTGG GGTACACAAA GTAAGCCTCT TTATGAAACA ATTGAGGATA | 1260 |
| ATGATGTGAA AGGTTTTAAT GATGATGTTC TAGGTCAGAT AGTTCACTTT CTACTGAATA | 1320 |
| GACCAAAAGA AGAAAAATCA CAGCTGTTGG AAAACTGAAA AAGCATATTT GATTGAGAAC | 1380 |
| TGTGGGAATA TTTAAATTTT ACTGAAGGAA CAATAATGAT GAGATTTGTA ACTGTCAACT | 1440 |
| ATTAAATACA TTGATTTTTG AGACAAATAT TTCTTATGTC AACCTGTTAT TAGATCTCTT | 1500 |
| ACTCTGCTCA AATTCATCAC TGAAAGATTT AATTTTAGTT ACCTTTTGTT GATTTAAAAA | 1560 |
| TAATTGCATT TGTATATTGC TAACTGATAA GACAAATTGA GTTATTGAGC TATTAAATGC | 1620 |
| ACATTTTAAT ATAAATGCAG AAATCCCAAA TAAAATGCTA ACATACTGAA TTCAGTAATT | 1680 |
| AAAAGAACCC ACTGC | |

Seq ID NO: 16 Protein sequence:
Protein Accession #: NP_057724.1

| | |
|---|---|
| 1 11 21 31 41 51 | |
| \| \| \| \| \| \| | |
| MAAARCWRPL LRGPRLSLHT AANAAATATE TTSQDVAATP VARYPPIVAS MTADSKAARL | 60 |
| RR1ERWQATV HAAESVDEKL RILTKMQFMK YMVYPQTFAL NADRWYQYFT KTVFLSGLPP | 120 |
| PPAEPEPEPE PEPEPALDLA ALRAVACDCL LQEHFYLRRR RRVHRYEESE VISLPFLDQL | 180 |
| VSTLVGLLSP HNPALAAAAL DYRCPVHFYW VRGEEIIPRG HRRGRIDDLR YQIDDKPNNQ | 240 |
| IRISKQLAEF VPLDYSVPIE IPTIKCKPDK LPLFKRQYEN HIFVGSKTAD PCCYGHTQFH | 300 |
| LLPDKLRRER LLRQNCADQI EVVFRANAIA SLFAWTGAQA MYQGFWSEAD VTRPFVSQAV | 360 |
| ITDGKYFSFF CYQLNTLALT TQADQNNPRK NICWGTQSKP LYETIEDNDV KGFNDDVLLQ | 420 |
| IVHFLLNRPK EEKSQLLEN | |

Seq ID NO: 17 DNA sequence
Nucleic Acid Accession #: NP_025059.1
Coding sequence: 3-2150 (underlined sequences correspond to start and
stop codons)

| | |
|---|---|
| 1 11 21 31 41 51 | |
| \| \| \| \| \| \| | |
| GCATGAGCCT GGACTGCACC AGCCATATCG CGCTGGGTGC CGCTTCGCCA GCGCGCGAGG | 60 |
| AAACTTACGA TCATCTTTCG GAAGTCCCGG TCACGCGGGA GCAGTTAAAC CACTATCGGA | 120 |
| ATGTGGCTCA AAATGCTCGA AGTGAACTTG CAGCAACTTT GGTCAAATTT GAATGTGCTC | 180 |
| AGTCTGAGCT TCAAGACCTC CGATCCAAGA TGCTTfCTAA AGAAGTCTCC TGTCAAGAAC | 240 |
| TGAAAGCTGA AATGGAGAGC TACAAGGAAA ACAATGCCAG AAAATCATCT CTCCTTACCT | 300 |
| CTTTGAGAGA CAGAGTTCAG GAACTAGAAG AAGAATCAGC AGCACTTTCC ACTTCTAAAA | 360 |
| TCAGAACAGA AATCACAGCT CACGCTGCAA TCAAGGAGAA CCAGGAATTA AAGAAGAAAG | 420 |
| TGGTAGAGTT AAATGAAAAA TTACAAAAGT GTTCAAAAGA AAATGAGGAG AATAAGAAAC | 480 |
| AAGTTTCAAA GAATTGCAGG AAACATGAGG AATTTCTGAC TCAACTGCGT GACTGCTTGG | 540 |
| ATCCAGATGA GAGGAATGAC AAGGCATCAG ATGAAGATTT AATTTTAAAG CTTAGAGACC | 600 |
| TGCGCAAAGA AAATGAATTC GTGAAAGGAC AAATTGTTAT TCTTGAAGAG ACTATAAATG | 660 |
| TCCATGAGAT GGAAGCAAAA GCTAGCAGAG AAACGATCAT GAGGCTGGCT TCAGAAGTCA | 720 |
| ACAGAGAGCA GAAAAAAGCT GCCTCCTGTA CTGAAGAGAA AGAGAAGCTG AACCAGGACC | 780 |
| TGCTCAGTGC TGTAGAAGCA AAAGAAGCTC TTGAAAGGGA AGTTAAGATC TTCCAAGAAA | 840 |
| GGCTGCTTGC TGGCCAGCAG GTCTGGGATG CCTCAAAGCA GGAAGTGAGC CTCCTGAAGA | 900 |
| AAAGCTCTTC TGAGTTGGAG AAGAGTTTCA AGGCCAGTCA GGATGCAGTC ACAACCTCAC | 960 |
| AAAGCCAGTA CTCCTCATTT AGGGAGAAAA TCGCAGCCCT CCHTAGGGGC AGATTGAGCA | 1020 |
| TGACTGGGTC CACTGAGGAC ACCATTTHGG AGAAGATTCG AGAAATGGAC AGCCGGGAAG | 1080 |
| AAAGCAGGGA CCGGATGGTC TCCCAGCTTG AAGCCCAAAT ATCTGAGCTT GTTGAACAGT | 1140 |
| TGGGAAAGGA GTCTGGGTTT CACCAGAAAG CTCTCCAGAG GGCCCAGAAA GCAGAGAATA | 1200 |
| TGTTGGAGAC TCTTCAGGGT CAGCTGACAC ACCTGGAGGC AGAGCTGGTT TCTGGAGGTG | 1260 |
| TTTTGCGAGA CAACTTGAAT TTTGAGAAAC AAAAATATCT TAAATTTCTG GATCAGCTTT | 1320 |
| CTCAGAAAAT GAAGTTGGAC CAGATGGCTG CCGAACTTGG CTTTGACACG CGGCTGGACG | 1380 |
| TGGTTTTAGC TCGAACAGAG CAGCTGGTTC GTCTTGAGAG CAATGCAGTC ATTGAGAACA | 1440 |
| AGACCATTGC CCACAATTTG CAGAGAAAGC TAAAGACACA GAAAGAGAGA CTGGAGAGCA | 1500 |
| AAGAATTACA CATGAGCCTC CTCCGGCAGA AAATAGCCCA GCTGGAGGAG GAGAAGCAGG | 1560 |
| CAGGCACGGC CTTGGTGGTT GAGAGGGACA ACGCGCATCT TACCATCAGG AACTTGCAGA | 1620 |
| AGAAGGTGGA GAGGCTGCAG AAAGAGCTGA ACACGTGTCG AGACTTGCAC ACCGAGCTCA | 1680 |
| AAGCCAAACT GGCCGACACC AATGAACTGA AGATTAAAAC TTTGGAACAG ACTAAAGCCA | 1740 |
| TTGAAGATCT AAACAAATCC AGAGACCAAC TGGAGAAGAT GAAGGAGAAA GCTGAGAAAA | 1800 |
| AGCTCATGTC TGTCAAGTCA GAACTGGATA CCACAGAACA TGAGGCTAAG GAGAATAAAG | 1860 |
| AAAGGGCCAG AAACATGATA GAAGTGGTAA CCAGTGAAAT GAAGACACTA AAAAAATCTC | 1920 |
| TGGAAGAAGC AGAAAAGAGA GAAAAGCAGC TGGCAGACTT CAGGGAGGTG GTGTCGCAGA | 1980 |
| TGCTAGGCTT GAACGTGACC AGCCTTGCTC TTCCTGATTA TGAAATCATC AAGTGTCTTG | 2040 |
| AAAGATTGGT CCATTCACAT CAGCATCACT TTGTTACCTG TGCCTGCCTC AAAGATGTGA | 2100 |
| CTACTGGGCA AGAGAGGCAC CCACAAGGCC ATTTACAGCT TCTTCATTGA ACACTGTATC | 2160 |
| TCTTGAGAGA GGTGGCCATA AGACATGGCA CACAATTCCC AATTTCACAA ATTCCTCATG | 2220 |
| TCTTTGAGAT TTGATCAGTT TGTGAATATT TTATGCTTTG ATGATATAGT GAGAATGCAT | 2280 |
| CACTTGCAAA AACGATCTCA AAAGTGTCAG CCTTAGATAA ACGTCAGCAT TAAAAAACGC | 2340 |
| CAAAAAAAAA AAAAAAAAGC ATTTTAGGAT CCAGAAGAAT TCCACCAGAT TGCATGAGTT | 2400 |
| AGATTGGGAA ATGGGAGTGG GAGATAATAT TGGGAGGTAT CTATTTTAAG TCAGGGGCTT | 2460 |
| TACTAGCCGA TTTAGTTCTC ACAATAACCA TGTGGAGAAG CTGTGACATT TTTAATTTAC | 2520 |
| AACCTTTCTG GCGCTCAGAC ATAAAGTTAC CTATCCAAGG TTGCAGTTGG GTAGTGGTGG | 2580 |
| GACCAGGATG GACAACTCAT TGGCCCTGCC TCAAAAGCCA TACCTCTTCT CCTGCTATGC | 2640 |
| AGAATCTGTT TCTCCTGAAT CTCTGTGATG CTGGTGGGAA TTGTTTGCAT AGAGGAAGGA | 2700 |
| CAATAACCCT GCCATCGTTA GTTAATGTCC GGGCTGGTCA CAGTGGTTCA TGCCTGTAAT | 2760 |
| CCCAGCACTT TGGGAGTCCA AGGCAGGCAT ATCATTTGAG GTCAGGAGTT TAAGACCAGC | 2820 |
| CTGGCTAACA TAGTGAGACC CTGTTTCTAC TAAAAATACA AAAATAAGCC AGGTGTGGTG | 2880 |
| GTGCATGACT GTAATCCCAA CTACTCAGCA GGAGAAGCAC TTGAACCCAG GAGACGGAGG | 2940 |
| CGGCAGTGAG CGAAGATTGT GCCACTGCAC TCCAGCCTGG GCGACAGAGT GAAACTACAT | 3000 |
| CTCAGAAAAA AAAAAAAAAA AAAAAAAAAA AAGTTAATGT CCAAAAATGA CAGATTTACA | 3060 |
| AGTGTAAGCT ATATGATTTC TTCAAAAAGC AAAAGCAATA TACCTAATTC ATTTGGATCA | 3120 |
| AACTTACATA GGTCTCAGGT CCTGTAAGAA ACTTGCCTGT TCTAACTGTT GCTACCAGAT | 3180 |
| TATATCTGGT GGTAATTGTT AATGTTTCAG CAGGGCTGGT CTCAGTCCTT TAAAATGGAA | 3240 |
| AGCTTTATTT GGAGCCCAAC CCTATAAGAT GAAGAAATCC TATATAGTCT TATTCACCAA | 3300 |
| TATATCCAAT ACACCCACAG CAATGGTACC TTTTTAAGAT CAGGATTTTA TTATGAATTC | 3360 |
| CTGTCACTTT CTGTTTTCCA TTTAAATTTC TATTTTACAA ATTTTTCAGG GAATCATATT | 3420 |
| CTTAACTTCA CTGAGAAAAA TGTGTTACTC TTTTGGACAA TTTATCTTAT TTCTATCATA | 3480 |
| TAAGATGTAT TTTTTTATTG TCCTTAAAAG AAGCTCTAGC ATGAAATTAA AGGAAAGGGA | 3540 |
| AAGAAATAGA TCTGGTGCAC CCGAACATTA GGAGAAAATG AAAAATATAC AACCAACCGT | 3600 |
| TCGTGAGTCA TCAAAAAGTC AAAGTCAGCC TGGCCAACAT GGCAAAACTC CGTCTCTGCA | 3660 |
| AAAAATACAA AAATGAGCCC GGTATGTTGG CATATGCCTG TAATCCCAGC TACTCGAGAG | 3720 |
| GCTGAGACAC GAAAATTGCT TGAACCTGGG AGGCGGAGGT TGCAATGAGC CGAGATCGCG | 3780 |
| CTACTGCACT CCAGCCTGGG CAACAGAGAG AGACCTTGTC TCAAAAAACA ACAACAACAA | 3840 |
| AAAGTCAAAG TCATAATAAG CAAATTATTG GCTTCTTTCT TCTAGACYAA AAGAAATTAA | 3900 |
| AGAGATGAAA CAATCAATTG CAAGGGTCAA AACTAGATTG GATCTTGGTT TGAATGAAAA | 3960 |
| AAAGCATAAA ATAITCTTGC AATAATTGTA AAAATTTGAA TGTGGACTAA GTCCTAGATT | 4020 |
| ATATTAAAAT ATTTTTAATT TTTTAAGCGT GACAAATGCA CTGATTGTTA TACTTTAAAT | 4080 |
| AACTAAAAAT CTGAGAATCC ACAGTGCTAC AGACAATAAA TGATAAAATG GGAAAAAAAA | 4140 |
| AAAAAAAAAA A | |

Seq ID NO: 18 Protein sequence:
Protein Accession #: NP_079335.1

| | |
|---|---|
| 1 11 21 31 41 51 | |
| \| \| \| \| \| \| | |
| MSLDCTSHIA LGAASPAPEE TYDHLSEVPV TREQLNHYRN VAQNARSELA ATLVKFECAQ | 60 |
| SELQDLRSKM LSKEVSCQEL KAEMESYKEN NARXSSLLTS LRDRVQELEE ESAALSTSKI | 120 |
| RTEITAHAAI KENQELKKKV VELNEKLQKC SKENEENKKQ VSKNCRKLEE FLTQLRDCLD | 180 |
| PDERNDKASD EDLILKLRDL RKENEFVKGQ TVILEETINV LBMEAKASRE TIMRLASEVN | 240 |
| REQKKAASCT EEKEKLNQDL LSAVEAKEAL EREVKIFQER LLAGQQVWDA SKQEVSLLKK | 300 |
| SSSELEKSLK ASQDAVTTSQ SQYSSFREKI AALLRGRLSM TGSTEDTILE KIREMDSREE | 360 |
| SRDRMVSQLE AQISELVEQL GKESGFIQKA LQRAQKAENM LETLQGQLTH LEAELVSGGV | 420 |
| LRDNLNFEKQ KYLKFLDQLS QKMCLDQMAA ELGFDTRLDV VLARTEQLVR LESNAVIENK | 480 |
| TIAHNLQRKL KTQKERLESK ELHMSLLRQK IAQLEEECQA RTALVVERDN AHLTIRNLQK | 540 |
| KVERLQKELN TCRDLHTELK AKLADTNELK IKTLEQTKAI EDLNKSRDQL EKMKEKAEKK | 600 |
| LMSVKSELDT TEHEAKENKE RARNMIEVVT SEMKTLKKSL EEAEKREKQL ADFREVVSQM | 660 |
| LGLNVTSLAL PDYEIIKCLE RIVHSHQHIW VTCACLKDVT TGQERBPQGH LQLLH | |

Seq ID NO: 19 DNA sequeNucleic Acid Accession #: AF071552 NM_000662
Coding sequence: 4411-1313 (underlined sequences correspond to stsrt
and stop codons)

| | |
|---|---|
| 1 11 21 31 41 51 | |
| \| \| \| \| \| \| | |
| CTTTGTATAA GGCTCAGCTA AAAGGGAAAT TGAGTGGGTC AGGTACCACG GATACTATAC | 60 |
| ACTCTATTGC ATGATTCTCC TGCCTACATC AGAAGACGTT TATAAGCCTA TTTTAAAGGA | 120 |
| TACCAGTTGG AATCTCTCTT TTATTAATCA CCAAGAGAAC CATGAACAAG CTGTTTATCA | 180 |
| TTTGACTCAT CATTTAATCT TGATTTCCAG CTTCTCACAC TTGAAAGAAG ACATAATACA | 240 |
| TTTCTCACAG GATTCTGGGA CTATTAACTG AACTTATGTG TGTAAAAGGA ATTCATACAA | 300 |
| TGAAAGCACT AGAAATAATT ATTATACTTA TAACCATTGT ATTTTTACAT GTTTAAAATA | 360 |
| TAGCCATAAT TAGCCTACTC AAATCCAAGT GTAAAAGTAA AATGATTTGC TTTCGTTTTG | 420 |
| TTTTCCTTGC TTAGGGGATC ATGGACATTG AAGCATATCT TGAAAGAATT GGCTATAAGA | 480 |
| AGTCTAGGAA CAAATTGGAC TTGGAAACAT TAACTGACAT TCTTCAACAC CAGATCCGAG | 540 |
| CTGTTCCCTT TGAGAACCTT AACATCCATT GTGGGGATGC CATGGACTTA GGCGTAGAGG | 600 |
| CCATTTTTGA TCAAGTTGTG AGAAGAAATC GGGGTGGATG GTGTCTCCAG GTCAATCATC | 660 |
| TTCTGTACTG GGCTCTGACC ACTATTGGTT TTGAGACCAC GATGTTGGGA GGGTATGTTT | 720 |
| ACAGCACTCC AGCCAAAAAA TACAGCACTG GCATGATTCA CCTTCTCCTG CAGGTGACCA | 780 |
| TTGATGGCAG GAACTACATT GTCGATGCTG GGTTTGGACG CTCATACCAG ATGTGGCAGC | 840 |
| CTCTGGAGTT AATTTCTGGG AAGGATCAGC CTCAGGTGCC TTGTGTCTTC CGTTTGACGG | 900 |
| AAGAGAATGG ATTCTGGTAT CTAGACCAAA TCAGAAGGGA ACAGTACATT CCAAATGAAG | 960 |
| AATTTCTTCA TTCTGATCTC CTAGAAGACA GCAAATACCG AAAAATCTAC TCCTTTACTC | 1020 |
| TTAAGCCTCG AACAATTGAA GATTTTGAGT CTATGAATAC ATACCTGCAG ACATCTCCAT | 1080 |
| CATCTGTGTT TACTAGTAAA TCATTTTGTT CCTTGCAGAC CCCAGATGGG GTTCACTGTT | 1140 |
| TGGTGGGCTT CACCCTCACC CATAGGAGAT TCAATTATAA GGACAATACA GATCTAATAG | 1200 |
| AGTTCAAGAC TCTGAGTGAG GAAGAAATAG AAAAAGTGCT GAAAAATATA TTTAATATTT | 1260 |
| CCTTGCAGAG AAAGCTTGTG CCCAAACATG GTGATAGATT TTTTACTATT TAGAATAAGG | 1320 |
| AGTAAAACAA TCTTGTCTAT TTGTCATCCA GCTCACCAGT TATCAACTGA CGACCTATCA | 1380 |
| TGTATCTTCT GTACCCTTAC CTTATTTTGA AGAAAATCCT AGACATCAAA TCATTTCACC | 1440 |
| TATAAAAATG TCATCATATA TAATTAAACA GCTTTTTAAA GAAACATAAC CACAAACGTT | 1500 |
| TTCAAATAAT AATAATAATA ATAATAATAA ATGTATTTTA AAGATGGCCT GTGGTTATCT | 1560 |
| TGGAAATTGG TGATTTATGC TAGAAAGCTT TTAATGTTGG TTTATTGTTG AATTC | |

Seq ID NO: 20 Protein sequence:
Protein Accession #: NP_000653.1

| | |
|---|---|
| 1 11 21 31 41 51 | |
| \| \| \| \| \| \| | |
| MDIEAYLERI GYKKSRNKLD LETLTDILQH QIRAVPFENL NIHCGDAMDL GLEAIFDQVV | 60 |
| RRNRGGWCLQ VNHLLYWALT TIGFETTMLG GYVYSTPAKK YSTGMIHLLL QVTIDGRNYI | 120 |
| VDAGFGRSYQ MWQPLELISG KDQPQVPCVF RLTEENGFWY LDQIRREQYI PNEEFLHSDL | 180 |
| LEDSKYRKIY SFTLKPRTIE DFESMNTYLQ TSPSSVFTSK SFCSLQTPDG VHCLVGFTLT | 240 |
| HRRFNYKDNT DLIEFKTLSE EEIEKVLKNI FNISLQRKLV PKHGDRFFTI | |

Seq ID NO: 21 DNA sequeNucleic Acid Accession #: NM_003714
Coding sequence: 123-1031 (underlined sequences correspond to start
and stop codons)

| | |
|---|---|
| 1 11 21 31 41 51 | |
| \| \| \| \| \| \| | |
| CGGCACGAGC AAAAAGGAAG AGTGGGAGGA GGAGGGGAAG CGGCGAAGGA GGAAGAGGAG | 60 |
| GAGGAGGAAG AGGGGAGCAC AAAGGATCCA GGTCTCCCGA CGGGAGGTTT ATACCAAGAA | 120 |
| CCATGTGTGC CGAGCGGCTG GGCCAGTTCA TGACCCTGGC TTTGGTGTTG GCCACCTTTG | 180 |
| ACCCGGCGCG GGGGACCGAC GCCACCAACC CACCCGAGGG TCCCCAAGAC AGGAGCTCCC | 240 |
| AGCAGAAAGG CCGCCTGTCC CTGCAGAATA CAGCGGAGAT CCAGCACTGT TTGGTCAACG | 300 |
| CTGGCGATGT GGGGTGTGGC GTGTTTGAAT GTTTCGAGAA CAACTCTTGT GAGATTCGGG | 360 |
| GCTTACATGG GATTTGCATG ACTTTTCTGC ACAACGCTGG AAAATTTGAT GCCCAGGGCA | 420 |
| AGTCATTCAT CAAAGACGCC TTGAAATGTA AGGCCCACGC TCTGCGGCAC AGGTTCGGCT | 480 |
| GCATAAGCCG GAAGTGCCCG GCCATCAGGG AAATGGTGTC CCAGTTGCAG CGGGAATGCT | 540 |
| ACCTCAAGCA CGACCTGTGC GCGGCTGCCC AGGAGAACAC CCGGGTGATA GTGGAGATGA | 600 |
| TCCATTTCAA GGACTTGCTG CTGCACGAAC CCTACGTGGA CCTCGTGAAC TTGCTGCTGA | 660 |
| CCTGTGGGGA GGAGGTGAAG GAGGCCATCA CCCACAGCGT GCAGGTTCAG TGTGAGCAGA | 720 |
| ACTGGGGAAG CCTGTGCTCC ATCTTGAGCT TCTGCACCTC GGCCATCCAG AAGCCTCCCA | 780 |
| CGGCGCCCCC CGAGCGCCAG CCCCAGGTGG ACAGAACCAA GCTCTCCAGG GCCCACCACG | 840 |
| GGGAAGCAGG ACATCACCTC CCAGAGCCCA GCAGTAGGGA GACTGGCCGA GGTGCCAAGG | 900 |
| GTGAGCGAGG TAGCAAGAGC CACCCAAACG CCCATGCCCG AGGCAGAGTC GGGGGCCTTG | 960 |
| GGGCTCAGGG ACCTTCCGGA AGCAGCGAGT GGGAAGACGA ACAGTCTGAG TATTCTGATA | 1020 |
| TCCGGAGGTG AAATGAAAGG CCTGGCCACG AAATCTTTCC TCCACGCCGT CCATTTTCTT | 1080 |
| ATCTATGGAC ATTCCAAAAC ATTTACCATT AGAGAGGGGG GATGTCACAC GCAGGATTCT | 1140 |
| GTGGGGACTG TGGACTTCAT CGAGGTGTGT GTTCGCGGAA CGGACAGGTG AGATGGAGAC | 1200 |
| CCCTGGGGCC GTGGGGTCTC AGGGGTGCCT GGTGAATTCT GCACTTACAC GTACTCAAGG | 1260 |
| GAGCGCGCCC GCGTTATCCT CGTACCTTTG TCTTCTTTCC ATCTGTGGAG TCAGTGGGTG | 1320 |
| TCGGCCGCTC TGTTGTGGGG GAGGTGAACC AGGGAGGGGC AGGGCAAGGC AGGGCCCCCA | 1380 |
| GAGCTGGGCC ACACAGTGGG TGCTGGGCCT CGCCCCGAAG CTTCTGGTGC AGCAGCCTCT | 1440 |
| GGTGCTGTCT CCGCGGAAGT CAGGGCGGCT GGATTCCAGG ACAGGAGTGA ATGTAAAAAT | 1500 |
| AAATATCGCT TAGAATGCAG GAGAAGGGTG GAGAGGAGGC AGGGGCCGAG GGGGTGCTTG | 1560 |
| GTGCCAAACT GAAATTCAGT TTCTTGTGTG GGGCCTTGCG GTTCAGAGCT CTTGGCGAGG | 1620 |
| GTGGAGGGAG GAGTGTCATT TCTATGTGTA ATTTCTGAGC CATTGTACTG TCTGGGCTGG | 1680 |
| GGGGGACACT GTCCAAGGGA GTGGCCCCTA TGAGTTTATA TTTTAACCAC TGCTTCAAAT | 1740 |
| CTCGATTTCA CTTTTTTTAT TTATCCAGTT ATATCTACAT ATCTGTCATC TAAATAAATG | 1800 |
| GCTTTCAAAC AAAAAAAAAA AAAAAAAAAA AAAAAAA | |

Seq ID NO: 22 Protein sequence:
Protein Accession #: NP_003705

| | |
|---|---|
| 1 11 21 31 41 51 | |
| \| \| \| \| \| \| | |
| MCAERIGQFM TLALVLATFD PARGTDATNP PEGPQDRSSQ QKGRLSLQNT AEIQHCLVNA | 60 |
| GDVGCGVEEC FENNSCEIRG LHGICMTFLH NAGKFDAQGK SFIKDALKCK AHALRHRFGC | 120 |
| ISRKCPAIRE MVSQLQRECY LKHDLCAAAQ ENTRVIVEMI HFKDLLLHEP YVDLVNLLLT | 180 |
| CGEEVKBAIT HSVQVQCEQN WGSLCSILSE CTSAIQKPPT APPERQPQVD RTKLSRAHHG | 240. |
| EAGHHLPEPS SRETGRGAKG ERGSKSHPNA HARGRVGGLG AQGPSGSSEW EDEQSEYSDI | 300 |
| RR | |

Seq ID NO: 23 DNA sequence
Nucleic Acid Accession #: NM_005264.1
Coding sequence: 557-1954 (underlined sequences correspond to start
and stop codons)

| | |
|---|---|
| 1 11 21 31 41 51 | |
| \| \| \| \| \| \| | |
| GAATTCCGGC CAGAAGAAAT CTGGCCTCGG AACACGCCAT TCTCCGCGCC GCTTCCAATA | 60 |
| ACCACTAACA TCCCTAACGA GCATCCGAGC CGAGGGCTCT GCTCGGAAAT CGTCCTGGCC | 120 |
| CAACTCGGCC CTTCGAGCTC TCGAAGATTA CCGCATCTAT TTTTYTTTTC TTTTTTTTCT | 180 |
| TTTCCTAGCG CAGATAAAGT GAGCCCGGAA AGGGAAGGAG GGGGCGGGGA CACCATTGCC | 240 |
| CTGAAAGAAT AAATAAGTAA ATAAACAAAC TGGCTCCTCG CCGCAGCTGG ACGCGGTCGG | 300 |
| TTGAGTCCAG GTTGGGTCGG ACCTGAACCC CTAAAAGCGG AACCGCCTCC CGCCCTCGCC | 360 |
| ATCCCGGAGC TGAGTCGCCG GCGGCGGTGG CTGCTGCCAG ACCCGGAGTT TCCTCTTTCA | 420 |
| CTGGATGGAG CTGAACTTTG GGCGGCCAGA GCAGCACAGC TGTCCGGGGA TCGCTGCACG | 480 |
| CTGAGCTCCC TCGGCAAGAC CCAGCGGCGG CTCGGGATTT TTTTGGGGGG GCGGGGACCA | 540 |
| GCCCCGCGCC GGCACCATGT TCCTGGCGAC CCTGTACTTC GCGCTGCCGC TCTTGGACTT | 600 |
| GCTCCTGTCG GCCGAAGTGA GCGGCGGAGA CCGCCTGGAT TGCGTGAAAG CCAGTGATCA | 660 |
| GTGCCTGAAG GAGCAGAGCT GCAGCACCAA GTACCGCACG CTAAGGCAGT GCGTGGCGGG | 720 |
| CAAGGAGACC AACTTCAGCC TGGCATCCGG CCTGGAGGCC AAGGATGAGT GCCGCAGCGC | 780 |
| CATGGAGGCC CTGAAGCAGA AGTCGCTCTA CAACTGCCGC TGCAAGCGGG GTATGAAGAA | 840 |
| GGAGAAGAAC TGCCTGCGCA TTTACTGGAG CATGTACCAG AGCCTGCAGG GAAATGATCT | 900 |
| GCTGGAGGAT TCCCCATATG AACCAGTTAA CAGGAGATTG TCAGATATAT TCCGGGTGGT | 960 |
| CCCATTCATA TCAGATGTTT TTCAGCAAGT GGAGCACATT CCCAAAGGGA ACAACTGCCT | 1020 |
| GGATGCAGCG AAGGCCTGCA ACCTCGACGA CATTTGCAAG AAGTACAGGT CGGCGTACAT | 1080 |
| CACCCCGTGC ACCACCAGCG TGTCCAACGA TGTCTGCAAC CGCCGCAAGT GCCACAAGGC | 1140 |
| CCTCCGGCAG TTC1TTGACA AGGTCCCGGC CAAGCACAGC TACGGAATGC TCTTCTGCTC | 1200 |
| CTGCCGGGAC ATCGCCTGCA CAGAGCGGAG GCGACAGACC ATCGTGCCTG TGTGCTCCTA | 1260 |
| TGAAGAGAGG GAGAAGCCCA ACTGTTTGAA TTTGCAGGAC TCCTGCAAGA CGAATTACAT | 1320 |
| CTGCAGATCT CGCCTTGCGG ATTTTTTTAC CAACTGCCAG CCAGAGTCAA GGTCTGTCAG | 1380 |
| CAGCTGTCTA AAGGAAAACT ACGCTGACTG CCTCCTCGCC TACTCGGGGC TTATTGGCAC | 1440 |
| AGTCATGACC CCCAACTACA TAGACTCCAG TAGCCTCAGT GTGGCCCCAT GGTGTGACTG | 1500 |
| CAGCAACAGT GGGAACGACC TAGAAGAGTG CTTGAAATTT TTGAATTTCT TCAAGGACAA | 1560 |
| TACATGTCTT AAAAATGCAA TTCAAGCCTT TGGCAATGGC TCCGATGTGA CCGTGTGGCA | 1620 |
| GCCAGCCTTC CCAGTACAGA CCACCACTGC CACTACCACC ACTGCCCTCC GGGTTAAGAA | 1680 |
| CAAGCCCCTG GGGCCAGCAG GGTCTGAGAA TGAAATTCCC ACTCATGTTT TGCCACCGTG | 1740 |
| TGCAAATTTA CAGGCACAGA AGCTGAAATC CAATGTGTCG GGCAATACAC ACCTCTGTAT | 1800 |
| TTCCAATGGT AATTATGAAA AAGAAGGTCT CGGTGCTTCC AGCCACATAA CCACAAAATC | 1860 |
| AATGGCTGCT CCTCCAAGCT GTGGTCTGAG CCCACTGCTG GTCCTGGTGG TAACCGCTCT | 1920 |
| GTCCACCCTA TTATCTTTAA CAGAAACATC ATAGCTGCAT TAAAAAAATA CAATATGGAC | 1980 |
| ATGTAAAAAG ACAAAAACCA AGTTATCTGT TTCCTGGGCT CTTGTATAGC TGAAATTCCA | 2040 |
| GTTTAGGAGC TCAGTTGAGA AACAGTTCCA TTCAACTGGA ACATTTTTTT TTTTCCTTTT | 2100 |
| AAGAAAGCTT CTTGTGATCC TTCGGGGCTT CTGTGAAAAA CCTGATGCAG TGCTCCATCC | 2160 |
| AAACTCAGAA GGCTTTGGGA TATGCTGTAT TTTAAAGGGA CAGTTTGTAA CTTGGGCTGT | 2220 |
| AAAGCAAACT GGGGCTGTGT TTTCGATGAT GATGATCATC ATGATCATGA TGATTYTAAC | 2280 |
| AGTTTTACTT CTGGCCTTTC CTAGCTAGAG AAGGAGTTAA TATTTCTAAG GTAACTCCCA | 2340 |
| TATCTCCTTT AATGACATTG ATTTCTAATG ATATAAATTT CAGCCTACAT TGATGCCAAG | 2400 |
| CTTTTTTGCC ACAAAGAAGA TTCTTACCAA GAGTGGGCTT TGTGGAAACA GCTGGTACTG | 2460 |
| ATGTTCACCT TTATATATGT ACTAGCATTT TCCACGCTGA TGTTTATGTA CTGTAAACAG | 2520 |
| TTCTGCACTC TTGTACAAAA GAAAAAACCA CCCGGAATTC | |

Seq ID NO: 24 Protein sequence:
Protein Accession #: NP_005255

| | |
|---|---|
| 1 11 21 31 41 51 | |
| \| \| \| \| \| \| | |
| MFLATLYFAL PLLDLLLSAE VSGGDRLDCV KASDQCLKEQ SCSTKYRTLR QCVAGKETNF | 60 |
| SLASGLEAKD ECRSAMEALK QKSLYNCRCK RGMKKEKNCL R1YWSMYQSL QGNDLLEDSP | 120 |
| YEPVNSRLSD IFRVVPFISD VFQQVEHIPK GNNCLDAAKA CNLDDICKKY RSAYITPCTT | 180 |
| SVSNDVCNRR KCHKALRQFF DKVPAKHSYG MLFCSCRDIA CTERRRQTIV PVCSYEEREK | 240 |
| PNCLNLQDSC KTNYICRSRL ADFFTNCQPE SRSVSSCLKE NYADCLLAYS GLIGTVMTPN | 300 |
| YIDSSSLSVA PWCDCSNSGN DLEECLKFLN FFKDNTCLKN AIQAFGNGSD VTVWQPAFPV | 360 |
| QTTTATTTTA LRVKNKPLGP AGSENEIPTH VLPPCANLQA QKLKSNVSGN THLCISNGNY | 420 |
| EKEGLGASSH I1TKSMAAPP SCGLSPLLVL VVTALSTLLS LTETS | |

Seq ID NO: 25 DNA sequence
Nucleic Acid Accession #: FGHNESH predicted
Coding sequence: 1576 (entire sequence represents open reading flame)

| | |
|---|---|
| 1 11 21 31 41 51 | |
| \| \| \| \| \| \| | |
| CTTTTGTTTC GCCATGCCTA GTCTAGTGGT ATCTGGAATA ATGGAAAGAA ATGGGGGCTT | 60 |
| TGGAGAACTA GGATGTTTCG GGGGAAGCGC TAAGGACCGA GGGCTGCTGG AAGACGAGCG | 120 |
| CGCCCTTCAG CTGGCTCTCG ATCAACTCTG CCTCCTGGGT TTGGGGGAGC CCCCCGCCCC | 180 |
| CAGGGCGGGC GAGGACGGGG GAGGTGGGGG GGGCGGCGCC CCCGCGCAGC CGACAGCCCC | 240 |
| CCCGCAGCCG GCGCCGCCGC CGCCGCCCGC GGCGCCCCCG GCCGCCCCGA CGACGGCCCC | 300 |
| CGCAGCGCAG ACGCCCCAGC CCCCCACCGC CCCCAAAGGG GCGAGCGACG CCAAGCTCTG | 360 |
| CGCTCTCTAC AAAGAGGCCG AGCTGCGCCT GAAGGGCAGC AGCAACACCA CGGAGTGTGT | 420 |
| TCCCGTGCCC ACCTCCGAGC ACGTGGCCGA GATCGTGGGC AGGCAAGGCT GCAAGATTAA | 480 |
| GGCCTTGAGG GCCAAGACCA ACACCTACAT CAAGACACCG GTGAGGGGCG AGGAACCAGT | 540 |
| GTTCATGGTG ACAGGGCGAC GGGAGGACGT GGCCACAGCC CGGCGGGAAA TCATCTCAGC | 600 |
| AGCGGAGCAC TTCTCCATGA TCCGTGCCTC CCGCAACAAG TCAGGCGCCG CCTTTGGTGT | 660 |
| GGCTCCTGCT CTGCCCGGCC AGGTGACCAT CCGTGTGCGG GTGCCCTACC GCGTGGTGGG | 720 |
| GCTGGTGGTG GGCCCCAAAG GGGCAACCAT CAAGCGCATC CAGCAGCAAA CCAACACATA | 780 |
| CATTATCACA CCAAGCCGTG ACCGCGACCC CGTGTTCGAG ATCACGGGTG CCCCAGGCAA | 840 |
| CGTGGAGCGT GCGCGCGAGG AGATCGAGAC GCACATCGCG GTGCGCACTG GCAAGATCCT | 900 |
| CGAGTACAAC AATGAAAACG ACTTCCTGGC GGGGAGCCCC GACGCAGCAA TCGATAGCCG | 960 |
| CTACTCCGAC GCCTGGCGGG TGCACCAGCC CGGCTGCAAG CCCCTCTCCA CCTTCCGGCA | 1020 |
| GAACAGCCTG GGCTGCATCG GCGAGTGCGG AGTGGACTCT GGCTTTGAGG CCCCACGCCT | 1080 |
| GGGTGAGCAG GGCGGGGACT TTGGCTACGG CGGGTACCTC TTTCCGGGCT ATGGCGTGGG | 1140 |
| CAAGCAGGAT GTGTACTACG GCGTGGCCGA GACTAGCCCC CCGCTGTGGG CGGGCCAGGA | 1200 |
| GAACGCCACG CCCACCTCCG TGCTCTTCTC CTCTGCCTCC TCCTCCTCCT CCTCTTCCGC | 1260 |
| CAAGGCCCGC GCTGGGCCCC CGGGCGCACA CCGCTCCCCT GCCACTTCCG CGGGACCCGA | 1320 |
| GCTGGCCGGA CTCCCGAGGC GCCCCCCGGG AGAGCCGCTC CAGGGCTTCT CTAAACTTGG | 1380 |
| TGGGGGCGGC CTGCGGAGCC CCGGCGGCGG GCGGGATTGC ATGGTCTGCT TTGAGAGCGA | 1440 |
| AGTGACTGCC GCCCTTGTGC CCTGCGGACA CAACCTGTTC TGCATGGAGT GTGCAGTACG | 1500 |
| CATCTGCGAG AGGACGGACC CAGAGTGTCC CGTCTGCCAC ATCACAGCCA CGCAAGCCAT | 1560 |
| CCGAATATTC TCCTAA | |

Seq ID NO: 26 Protein sequence:
Protein Accession #: FGENESH predicted

| | |
|---|---|
| 1 11 21 31 41 51 | |
| \| \| \| \| \| \| | |
| FCFAMPSLVV SGIMERNGGF GELGCFGGSA KDRGLLEDER ALQLALDQLC LLGLGEPPAP | 60 |
| RAGEDGGGGG GGAPAQPTAP PQPAPPPPPA APPAAPTTAP AAQTPQPPTA PKGASDAXLC | 120 |
| ALYKEAELRL KGSSNTTECV PVPTSEHVAE IVGRQGCKIK ALRAKTNTYI KTPVRGEEPV | 180 |
| FMVTGRREDV ATARREIISA AEHFSMIRAS RNKSGAAFGV APALPGQVTI RVRVPYRVVG | 240 |
| LVVGPKGATI KRIQQQTNTY IITPSRDRDP VFEITGAPGN VERARBEIET HIAVRTGKIL | 300 |
| EYNNENDFLA GSPDAAIDSR YSDAWRVHQP GCKPLSTFRQ NSLGCIGEGG VDSGFEAPRL | 360 |
| GEQGGDFGYG GYLEPGYGVG KQDVYYGVAE TSPPLWAGQE NATPTSVLFS SASSSSSSSA | 420 |
| KARAGPPGAH RSPATSAGPE LAGLPRRPPG EPLQGFSKLG GGGLRSPGGG RDCMVCFESE | 480 |
| VTAALVPCGH NLFCMECAVR 1CERTDPECP VCHITATQAI RIFS | |

Seq ID NO: 27 DNA sequeNucleic Acid Accession #: FGENESH predicted
Coding sequence: 1-2070 (underlined sequences correspond to start and
stop codons)

| | |
|---|---|
| 1 11 21 31 41 51 | |
| \| \| \| \| \| \| | |
| ATGAGCGGTG CGGGGGTGGC GGCTGGGACG CGGCCCCCCA GCTCGCCGAC CCCGGGCTCT | 60 |
| CGGCGCCGGC GCCAGCGCCC CTCTGTGGGC GTCCAGTCCT TGAGGCCGCA GAGCCCGCAG | 120 |
| CTCAGGCAGA GCGACCCGCA GAAACGGAAC CTGGACCTGG AGAAAAGCCT GCAGTTCCTG | 180 |
| CAGCAGCAGC ACTCGGAGAT GCTGGCCAAG CTCCATGAGG AGATCGAGCA TCTGAAGCGG | 240 |
| GAAAACAAGG GTGAGCCGGC GCGGGGCCCT AGGCCGGCCC TGCCTCCCCA GGCACACTCA | 300 |
| ACACTGCCGC TCCCGCAGCA CAGAAACACA GCCATCAACT CCAGCACACG CCTGGGCTCA | 360 |
| GGGGGAACAC AGGACGGGGA GCCCCTCCAG ACTGTCCTTG CCCACCTGGC TGCACTGGCC | 420 |
| CCTGTATGCC AACCCAGTGG GTACAGGTTC TGGGGGACCT GGACAGATGC CGCTACCTCT | 480 |
| AGCCGTGGCT GGACGATGTT ATGCAGCCAA GCACAGCACG TGCTGCTCTC GGGAAGCCCA | 540 |
| GGGCCTGAGG TCATTGCAGG GCGGCAGGTG GCCACAGGGT GCTCCCCAGA CCTCCCTCCT | 600 |
| CCAAGTAGAG CTGAAATGGG AAGGAACCCC TGGGACAGCC CCTGCCCTGC TAGATCTTTG | 660 |
| CCTCAGATTG CTGCTGTGGC CAGGCCCAGG ATTTCCAGCC CTATGGCTCT GAGTCCTCAC | 720 |
| ATGCTGGGGG CCCAGGGGAT ATGGACACAC TCCATCCAGG GATCCCTTCC TGCCATCTGG | 780 |
| GCAGCAACCA TGGGGACAAA GGGAGGAAGC AGAGTCCTGT TTCCTTGCCA GTTGTCCAAG | 840 |
| GCACTfCCCC ATCCTGACAG CGGCCCCCAC CCAGCCCAGG ATCCTGGGCT GTGGTCTCAA | 900 |
| GCTCACTTCC CATTATCTTT GGGGCTGGGG CTGACATCAG GAGGACATCT GACTGGTGGA | 960 |
| TGGAGCCAGC CTGGGAACAT CGCAGCTGGG GCAGTGCCTA GGGCTCTCCC TTCCCAGGGA | 1020 |
| GACATGGAGA AGGGGGTTGA GGGAGGGCCC TTCCCTAGCC GCTGTGGCAA CTCCAGTGAG | 1080 |
| CTGTTCTGGG CAAAGTGTGG CCCAAGTCGG CAGCCCCAGC CCTGCAGTGC TGGGGACGCT | 1140 |
| GACAGGACAC GGGAAGAGGC CATGCTTTCC CTCGGGACCT GCTGTTCCAT GTGTCCCAAG | 1200 |
| CCCTCCTGCT TTCCAGATGG CCCCTCAGGA AACCACCTTT CCAGGGCCTC TGCTCCCTTG | 1260 |
| GGCGCTCGCT GGGTCTGCAT CAACGGAGTG TGGGTAGAGC CGGGAGGACC CAGCCCTGCC | 1320 |
| AGGCTGAAGG AGGGCTCCTC ACGGACACAC AGGCCAGGAG GCAAGCGTGG GCGTCTTGCG | 1380 |
| GGCGGTAGCG CCGACACTGT GCGCTCTCCT GCAGACAGCC TCTCCATGTC AAGCTTCCAG | 1440 |
| TCTGTCAAGT CCATCTCTAA TTCAGCCAAC TCTCAAGGCA AGGCCAGGCC CCAGCCCGGC | 1500 |
| TCCTTCAACA AGCAAGATTC AAAAGCTGAC GTCTCCCAGA AGGCGGACCT GGAAGAGGAG | 1560 |
| CCCCTACTTC ACAACAGCAA GCTGGACAAA GTTCCTGGGG TACAAGGGCA GGCCAGAAAG | 1620 |
| GAGAAAGCAG AGGCCTCTAA TGCAGGAGCT GCCTGTATGG GGAACAGCCA GCACCAGGGC | 1680 |
| AGGCAGATGG GGGCGGGGGC ACACCCCCCA ATGATCCTGC CCCTTCCCCT GCGAAAGCCC | 1740 |
| ACCACACTTA GGCAGTGCGA AGTGCTCATC CGCGAGCTGT GGAATACCAA CCTCCTGCAG | 1800 |
| ACCCAAGAGC TGCGGCACCT CAAGTCCCTC CTGGAAGGGA GCCAGAGGCC CCAGGCAGCC | 1860 |
| CCGGAGGAAG CTAGCTTTCC CAGGGACCAA GAAGCCACGC ATTTCCCCAA GGTCTCCACC | 1920 |
| AAGAGCCTCT CCAAGAAATG CCTGAGCCCA CCTGTGGCGG AGCGTGCCAT CCTGCCCGCA | 1980 |
| CTGAAGCAGA CCCCGAAGAA CAACTTTGCC GAGAGGCAGA AGAGGCTGCA GGCAATGCAG | 2040 |
| AAACGGCGCC TGCATCGCTC AGTGCTTTGA | |

Seq ID NO: 28 Protein sequence:
Protein Accession #: EGENESH predicted

| | |
|---|---|
| 1 11 21 31 41 51 | |
| \| \| \| \| \| \| | |
| MSGAGVAAGT RPPSSPTPGS RRRRQRPSVG VQSLRPQSPQ LRQSDPQKRN LDLEKSLQFL | 60 |
| QQQHSEMLAK LHEEIEHLKR ENKGEPARGP RPALPPQAHS TLPLPQHRNT AINSSTRLGS | 120 |
| GGTQDGEPLQ TVLAHLAALA PVCQPSGYRF WGTWTDAATS SRGWTMLCSQ AQHVLLSGSP | 180 |
| GPEVIAGRQV ATGCSPDLPP PSRAEMGRNP WDSPCPARSL PQIAAVARTR ISSPMALSPH | 240 |
| MLGAQGIWTH SIQGSLPAIW AATMGTKGGS RVLFPCHLSK ALPHPDSGPH PAQDPGLWSQ | 300 |
| AHFPLSLGLG LTSGGHLTGG WSQPGNIAAG AVPRALPSQG DMEKGVEGGP FPSRCGNSSE | 360 |
| LFWAKCGPSR QPQPCSAGDA DRTREEAMLS LGTCCSMCPK PSCFPDGPSG NHLSRASAPL | 420 |
| GARWVCINGV WVEPGGPSPA RLKEGSSRTH RPGGKRGRLA GGSADTVRSP ADSLSMSSFQ | 480 |
| SVKSISNSAN SQGKARPQPG SFNKQDSKAD VSQKADLEEE PLLHNSKLDK VPGVQGQARK | 540 |
| EKAEASNAGA ACMGNSQHQG RQMGAGAHPP MILPLPLRKP TTLRQCEVLI RELWNTNLLQ | 600 |
| TQELRHLKSL LEGSQRPQAA PEEASFPRDQ EATHFPKVST KSLSKKCLSP PVAERAILPA | 660 |
| LKQTPKNNFA ERQKRLQAMQ KRRLHRSVL | |

Seq ID NO: 29 DNA sequeNucleic Acid Accession #: NM_012319.2
Coding sequence: 138-2405 (underlined sequences correspond to start
and stop codons)

| | |
|---|---|
| 1 11 21 31 41 51 | |
| \| \| \| \| \| \| | |
| CTCGTGCCGA ATTCGGCACG AGACCGCGTG TTCGCGCCTG GTAGAGATTT CTCGAAGACA | 60 |
| CCAGTGGGCC CGTGTGGAAC CAAACCTGCG CGCGTGGCCG GGCCGTGGGA CAACGAGGCC | 120 |
| GCGGAGACGA AGGCGCAATG GCGAGGAAGT TATCTGTAAT CTTGATCCTG ACCTTTGCCC | 180 |
| TCTCTGTCAC AAATCCCCTT CATGAACTAA AAGCAGCTGC TTTCCCCCAG ACCACTGAGA | 240 |
| AAATTAGTCC GAATTGGGAA TCTGGCATTA ATGTTGACTT GGCAATTTCC ACACGGCAAT | 300 |
| ATCATCTACA ACAGCTTTTC TACCGCTATG GAGAAAATAA TTCTTTGTCA GTTGAAGGGT | 360 |
| TCAGAAAATT ACTTCAAAAT ATAGGCATAG ATAAGATTAA AAGAATCCAT ATACACCATG | 420 |
| ACCACGACCA TCACTCAGAC CACGAGCATC ACTCAGACCA TGAGCGTCAC TCAGACCATG | 480 |
| AGCATCACTC AGACCACGAG CATCACTCTG ACCATGATCA TCACTCTCAC CATAATCATG | 540 |
| CTGCTTCTGG TAAAAATAAG CGAAAAGCTC TTTGCCCAGA CCATGACTCA GATAGTTCAG | 600 |
| GTAAAGATCC TAGAAACAGC CAGGGGAAAG GAGCTCACCG ACCAGAACAT GCCAGTGGTA | 660 |
| GAAGGAATGT CAAGGACAGT GTTAGTGCTA GTGAAGTGAC CTCAACTGTG TACAACACTG | 720 |
| TCTCTGAAGG AACTCACTTT CTAGAGACAA TAGAGACTCC AAGACCTGGA AAACTCTTCC | 780 |
| CCAAAGATGT AAGCAGCTCC ACTCCACCCA GTGTCACATC AAAGAGCCGG GTGAGCCGGC | 840 |
| TGGCTGGTAG GAAAACAAAT GAATCTGTGA GTGAGCCCCG AAAAGGCTTT ATGTATTCCA | 900 |
| GAAACACAAA TGAAAATCCT CAGGAGTGTT TCAATGCATC AAAGCTACTG ACATCTCATG | 960 |
| GCATGGGCAT CCAGGTTCCG CTGAATGCAA CAGAGTTCAA CTATCTCTGT CCAGCCATCA | 1020 |
| TCAACCAAAT TGATGCTAGA TCTTGTCTGA TTCATACAAG TGAAAAGAAG GCTGAAATCC | 1080 |
| CTCCAAAGAC CTAGGCATTA CAAATAGCCT GGGTTGGTGG TTTTATAGCC ATTTCCATCA | 1140 |
| TCAGTTTCCT GTCTCTGCTG GGGGTTATCT TAGTGCCTCT CATGAATCGG GTGTTTTTCA | 1200 |
| AATTTCTCCT GAGTTTCCTT GTGGCACTGG CCGTTGGGAC TTTGAGTGGT GATGCTTTTT | 1260 |
| TACACCTTCT TCCACATTCT CATGCAAGTC ACCACCATAG TCATAGCCAT GAAGAACCAG | 1320 |
| CAATGGAAAT GAAAAGAGGA CCACTTTTCA GTCATCTGTC TTCTCAAAAC ATAGAAGAAA | 1380 |
| GTGCCTATTT TGATTCCACG TGGAAGGGTC TAACAGCTCT AGGAGGCCTG TATTTCATGT | 1440 |
| TTCTTGTTGA ACATGTCCTC ACATTGATCA AACAATTTAA AGATAAGAAG AAAAAGAATC | 1500 |
| AGAAGAAACC TGAAAATGAT GATGATGTGG AGATTAAGAA GCAGTTGTCC AAGTATGAAT | 1560 |
| CTCAACTTTC AACAAATGAG GAGAAAGTAG ATACAGATGA TCGAACTGAA GGCTATTTAC | 1620 |
| GAGCAGACTC ACAAGAGCCC TCCCACTTTG ATTCTCAGCA GCCTGCAGTC TTGGAAGAAG | 1680 |
| AAGAGGTCAT GATAGCTCAT GCTCATCCAC AGGAAGTCTA CAATGAATAT GTACCCAGAG | 1740 |
| GGTGCAAGAA TAAATGCCAT TCACATTTCC ACGATACACT CGGCCAGTCA GACGATCTCA | 1800 |
| TTCACCACCA TCATGACTAC CATCATATTC TCCATCATCA CCACCACCAA AACCACCATC | 1860 |
| CTCACAGTCA CAGCCAGCGC TACTCTCGGG AGGAGCTGAA AGATGCCGGC GTCGCCACTT | 1920 |
| TGGCCTGGAT GGTGATAATG GGTGATGGCC TGCACAATTT CAGCGATGGC CTAGCAATTG | 1980 |
| GTGCTGCTTT TACTGAAGGC TTATCAAGTG GTTTAAGTAC TTCTGTTGCT GTGTTCTGTC | 2040 |
| ATGAGTTGCC TCATGAATTA GGTGACTTTG CTGTTCTACT AAAGGCTGGC ATGACCGTTA | 2100 |
| AGCAGGCTGT CCTTTATAAT GCA1TGTCAG CCATGCTGGC GTATCTTGGA ATGGCAACAG | 2160 |
| GAATTTTCAT TGGTCATTAT GCTGAAAATG TTTCTATGTG GATATfTGCA CTTACTGCTG | 2220 |
| GCTTATTCAT GTATG3TGCT CTGGTTGATA TGGTACCTGA AATGCTGCAC AATGATGCTA | 2280 |
| GTGACCATGG ATGTAGCCGC TGGGGGTATT TCTTTTTACA GAATGCTGGG ATGCTTTTGG | 2340 |
| GTTTTGGAAT TATGTTACTT ATTTCCATAT TTGAACATAA AATCGTGTTT CGTATAAATT | 2400 |
| TCTAGTTAAG GTTTAAATGC TAGAGTAGCT TAAAAAGTTG TCATAGTTTC AGTAGGTCAT | 2460 |
| AGGGAGATGA GTTTGTATGC TGTACTATGC AGCGTTfAAA GTTAGTGGGT TTTGTGATTT | 2520 |
| TTGTATTGAA TATTGCTGTC TGTTACAAAG TCAGTTAAAG GTACGTTTTA ATATTTAAGT | 2580 |
| TATTCTATCT TGGAGATAAA ATCTGTATGT GCAATTCACC GGTATTACCA GTTTATTATG | 2640 |
| TAAACAAGAG ATTTGGCATG ACATGTTCTG TATGTTTCAG GGAAAAATGT CTTTAATGCT | 2700 |
| TTTTCAAGAA CTAACACAGT TATTCCTATA CTGGATTTTA GGTCTCTGAA GAACTGCTGG | 2760 |
| TGTTTAGGAA TAAGAATGTG CATGAAGCCT AAAATACCAA GAAAGCTTAT ACTGAATTTA | 2820 |
| AGCAAAGAAA TAAAGGAGAA AAGAGAAGAA TCTGAGAATT GGGGAGGCAT AGATTCTTAT | 2880 |
| AAAAATCACA AAATTTGTTG TAAAITAGAG GGGAGAAATT TAGAATTAAG TATAAAAAGG | 2940 |
| CAGAATTAGT ATAGAGTACA TTCATTAAAC ATTTTTGTCA GGATTATTTC CCGTAAAAAC | 3000 |
| GTAGTGAGCA CTCTCATATA CTAATTAGTG TACATTTAAC TTTGTATAAT ACAGAAATCT | 3060 |
| AAATATATTT AATGAATTCA AGCAATATAC ACTTGACCAA GAAATTGGAA TTTCAAAATG | 3120 |
| TTCGTGCGGG TTATATACCA GATGAGTACA GTGAGTAGTT TATGTATCAC CAGACTGGGT | 3180 |
| TATTGCCAAG TTATATATCA CCAAAAGCTG TATGACTGGA TGTTCTGGTT ACCTGGTTTA | 3240 |
| CAAAATTATC AGAGTAGTAA AACTTTGATA TATATGAGGA TATTAAAACT ACACTAAGTA | 3300 |
| TCATTTGATT CGATTCAGAA AGTACTTTGA TATCTCTCAG TGCTTCAGTG CTATCATTGT | 3360 |
| GAGCAATTGT CTTTATATAC GGTACTGTAG CCATACTAGG CCTGTCTGTG GCATTCTCTA | 3420 |
| GATGTTTCTT TTTTACACAA TAAATTCCTT ATATCAGCTT G | |

Seq ID NO: 30 Protein sequence:
Protein Accession #: NP_036451.2

| | |
|---|---|
| 1 11 21 31 41 51 | |
| \| \| \| \| \| \| | |
| MARKLSVILI LTFALSVTNP LNELKAAAFP QTTEKISPNW ESGINVDLAI STRQYHLQQL | 60 |
| FYRYGENNSL SVEGFRKLLQ NIGIDKIKRI HIHHDHDHHS DHEHHSDHER HSDHEHHSDH | 120 |
| EHHSDHDHHS HHNHAASGKN KRKALCPDHD SDSSGKDPRN SQGKGAHRPE HASGRRNVKD | 180 |
| SVSASEVTST VYNTVSEGTH FLETIETPRF GKLFPKDVSS STPPSVTSKS RVSRLAGRKT | 240 |
| NESVSEPRXG FMYSRNTNEN PQECFNASKL LTSHGMGIQV PLNATEFNYL CPAIINQIDA | 300 |
| RSCLIHTSEK KAEIPPKTYS LQIAWVGGFI AISIISFLSL LGVILVPLMN RVEFKFLLSF | 360 |
| LVALAVGTLS GDAFLHLLPH SHASHHHSHS HEEPAMEMKR GPLFSHLSSQ NIEESAYFDS | 420 |
| TWKGLTALGG LYEMFLVEHV LTLIKQFKDK KKKNQKKPEN DDDVELKKQL SKYESQLSTN | 480 |
| EEKVDTDDRT EGYLRADSQE PSHFDSQQPA VLEEEEVMIA HAHPQEVYNE YVPRGCKNKC | 540 |
| HSHFHDTLGQ SDDLIHHHHD YHHILHHHHH QNHHPHSHSQ RYSREELKDA GVATLAWMVI | 600 |
| MGDGLHNFSD GLAIGAAFTE GLSSGLSTSV AVFCHELPHE LGDFAVLLKA GMTVKQAVLY | 660 |
| NALSAMLAYL GMATGIFIGH YAENVSMWIF ALTAGLFMYV ALVDMVPEML HNDASDHGCS | 720 |
| RWGYFFLQNA GMLLGFGIML LISIFEHKIV FRINF | |

Seq ID NO: 31 DNA sequence
Nucleic Acid Accession #: NM_002184.1
Coding sequence: 256-3012 (underlined sequences conespond to start snd
stop codons)

| | |
|---|---|
| 1 11 21 31 41 51 | |
| \| \| \| \| \| \| | |
| GAGCAGCCAA AAGGCCCGCG GAGTCGCGCT GGGCCGCCCC GGCGCAGCTG AACCGGGGGC | 60 |
| CGCGCCTGCC AGGCCGACGG GTCTGGCCCA GCCTGGCGCC AAGGGGTTCG TGCGCTGTGG | 120 |
| AGACGCGGAG GGTCGAGGCG GCGCGGCCTG AGTGAAACCC AATGGAAAAA GCATGACATT | 180 |
| TAGAAGTAGA AGACTTAGCT TCAAATCCCT ACTCCTTCAC TTACTAATTT TGTGATTTGG | 240 |
| AAATATCCGC GCAAGATGTT GACGTTGCAG ACTTGGGTAG TGCAAGCCTT GTTTATTTTC | 300 |
| CTCACCACTG AATCTACAGG TGAACTTCTA GATCCATGTG GTTATATCAG TCCTGAATCT | 360 |
| CCAGTTGTAC AACTTCATTC TAATTTCACT GCAGTTTGTG TGCTAAAGGA AAAATGTATG | 420 |
| GATTATTTTC ATGTAAATGC TAATTACATT GTCTGGAAAA CAAACCATTT TACTATTCCT | 480 |
| AAGGAGCAAT ATACTATCAT AAACAGAACA GCATCCAGTG TCACCTTTAC AGATATAGCT | 540 |
| TCATTAAATA TTCAGCTCAC TTGCAACATT CTTACATTCG GACAGCTTGA ACAGAATGTT | 600 |
| TATGGAATCA CAATAATTTC AGGCTTGCCT CCAGAAAAAC CTAAAAATTT GAGTTGCATT | 660 |
| GTGAACGAGG GGAAGAAAAT GAGGTGTGAG TGGGATGGTG GAAGGGAAAC ACACTTGGAG | 720 |
| ACAAACTTCA CTTTAAAATC TGAATGGGCA ACACACAAGT TTGCTGATTG CAAAGCAAAA | 780 |
| CGTGACACCC CCACCTCATG CACTGTTGAT TATTCTACTG TGTATTTTGT CAACATTGAA | 840 |
| GTCTGGGTAG AAGCAGAGAA TGCCCTTGGG AAGGTTACAT CAGATCATAT CAATTTTGAT | 900 |
| CCTGTATATA AAGTGAAGCC CAATCCGCCA CATAATTTAT CAGTGATCAA CTCAGAGGAA | 960 |
| CTGTCTAGTA TCTTAAAATT GACATGGACC AACCCAAGTA TTAAGAGTGT TATAATACTA | 1020 |
| AAATATAACA TTCAATATAG GACCAAAGAT GCCTCAACTT GGAGCCAGAT TCCTCCTGAA | 1080 |
| GACACAGCAT CCACCCGATC TTCATTCACT GTCCAAGACC TTAAACCTTT TACAGAATAT | 1140 |
| GTGTTTAGGA TTCGCTGTAT GAAGGAAGAT GGTAAGGGAT ACTGGAGTGA CTGGAGTGAA | 1200 |
| GAAGCAAGTG GGATCACCTA TGAAGATAGA CCATCTAAAG CACCAAGTTT CTGGTATAAA | 1260 |
| ATAGATCCAT CCCATACTCA AGGCTACAGA ACTGTACAAC TCGTGTGGAA GACATTGCCT | 1320 |
| CCTTTTGAAG CCAATGGAAA AATCTTGGAT TATGAAGTGA CTCTCACAAG ATGGAAATCA | 1380 |
| CATTTACAAA ATTACACAGT TAATGCCACA AAACTGACAG TAAATCTCAC AAATGATCGC | 1440 |
| TATCTAGCAA CCCTAACAGT AAGAAATCTT GTTGGCAAAT CAGATGCAGC TGTTTTAACT | 1500 |
| ATCCCTGCCT GTGACTTTCA AGCTACTCAC CCTGTAATGG ATCTTAAAGC ATTCCCCAAA | 1560 |
| GATAACATGC TTTGGGTGGA ATGGACTACT CCAAGGGAAT CTGTAAAGAA ATATATACTT | 1620 |
| GAGTGGTGTG TGTTATdAGA TAAAGCACCC TGTATCACAG ACTGGCAACA AGAAGATGGT | 1680 |
| ACCGTGCATC GCACCTATTT AAGAGGGAAC TTAGCAGAGA GCAAATGCTA TITGATAACA | 1740 |
| GTTACTCCAG TATATGCTGA TGGACCAGGA AGCCCTGAAT CCATAAAGGC ATACCTTAAA | 1800 |
| CAAGCTCCAC CTTCCAAAGG ACCTACTGTT CGGACAAAAA AAGTAGGGAA AAACGAAGCT | 1860 |
| GTCTTAGAGT GGGACCAACT TCCTGTTGAT GTTCAGAATG GATTTATCAG AAATTATACT | 1920 |
| ATAITITATA GAACCATCAT TGGAAATGAA ACTGCTGTGA ATGTGGATTC TTCCCACACA | 1980 |
| GAATATACAT TGTCCTCTTT GACTAGTGAC ACATTGTACA TGGTACGAAT GGCAGCATAC | 2040 |
| ACAGATGAAG GTGGGAAGGA TGGTCCAGAA TTCACTTTTA CTACCCCAAA GTTTGCTCAA | 2100 |
| GGAGAAATTG AAGCCATAGT CGTGCCTGTT TGCTTAGCAT TCCTATTGAC AACTCITCTG | 2160 |
| GGAGTGCTGT TCTGCTTTAA TAAGCGAGAC CTAATTAAAA AACACATCTG GCCTAATGTT | 2220 |
| CCAGATCCTT CAAAGAGTCA TATTGCCCAG TGGTCACCTC ACACTCCTCC AAGGCACAAT | 2280 |
| TTTAATTCAA AAGATCAAAT GTATTCAGAT GGCAATTTCA CTGATGTAAG TGTTGTGGAA | 2340 |
| ATAGAAGCAA ATGACAAAAA GCCTTTTCCA GAAGATCTGA AATCATTGGA CCTGTTCAAA | 2400 |
| AAGGAAAAAA TTAATACTGA AGGACACAGC AGTGGTATTG GGGGGTCTTC ATGCATGTCA | 2460 |
| TCTTCTAGGC CAAGCATTTC TAGCAGTGAT GAAAATGAAT GTTCACAAAA CACTTCGAGC | 2520 |
| ACTGTCCAGT A3TCTACCGT GGTACACAGT GGCTACAGAC ACCAAGTTCC GTCAGTCCAA | 2580 |
| GTCTTCTCAA GATCCGAGTC TACCCAGCCC TTGTTAGATT CAGAGGAGCG GCCAGAAGAT | 2640 |
| CTACAATTAG TAGATCATGT AGATGGCGGT GATGGTATTT TGCCCAGGCA ACAGTACTTC | 2700 |
| AAACAGAACT GCAGTCAGCA TGAATCCAGT CCAGATATTT CACATTTTGA AAGGTCAAAG | 2760 |
| CAAGTTTCAT CAGTCAATGA GGAAGATTTT GTTAGACTTA AACAGCAGAT TTCAGATCAT | 2820 |
| ATTTCACAAT CCTGTGGATC TGGGCAAATG AAAATGTTTC AGGAAGTTTC TGCAGCAGAT | 2880 |
| GCTTTTGGTC CAGGTACTGA GGGACAAGTA GAAAGATTTG AAACAGTTGG CATGGAGGCT | 2940 |
| GCGACTGATG AAGGCATGCC TAAAAGTTAC TTACCACAGA CTGTACGGCA AGGCGGCTAC | 3000 |
| ATGCCTCAGT GAAGGACTAG TAGTTCCTGC TACAACTTCA GCAGTACCTA TAAAGTAAAG | 3060 |
| CTAAAATGAT TTTATCTGTG AATTC | |

Seq ID NO: 32 Protein sequence:
Protein Accession #: NP_002175.1

| | |
|---|---|
| 1 11 21 31 41 51 | |
| \| \| \| \| \| \| | |
| MLTLQTWVVQ ALFIFLTTES TGELLDPCGY ISPESPVVQL HSNFTAVCVL KEKCMDYFHV | 60 |
| NANYIVWKTN HFTIPKEQYT IINRTASSVT FTDIASLNIQ LTCNILTFGQ LEQNVYGITI | 120 |
| ISGLPPEKPK NLSCIVNEGK KMRCEWDGGR ETHLETNETL KSEWATHKFA DCKAKRDTPT | 180 |
| SCTVDYSTVY FVNIEVWVEA ENALGKVTSD HINFDPVYKV KPNPPHNLSV INSEELSSIL | 240 |
| KLTWTNPSIK SVIILKYNIQ YRTKDASTWS QIPPEDTAST RSSFTVQDLK PFTEYVFRIR | 300 |
| CMKEDGKGYW SDWSEEASGI TYEDRPSKAP SFWYKIDPSH TQGYRTVQLV WKTLPPFEAN | 360 |
| GKILDYEVTL TRWKSHLQNY TVNATKLTVN LTNDRYLATL TVRNLVGKSD AAVLTIPACD | 420 |
| FQATHPVMDL KAFPKDNMLW VEWTTPRESV KKYILEWCVL SDKAPCITDW QQEDGTVHRT | 480 |
| YLRGNLAESK CYLITVTPVY ADGPGSPESI KAYLKQAPPS KGPTVRTKKV GKNEAVLEWD | 540 |
| QLPVDVQNGF IRNYTIFYRT IIGNETAVNV DSSHTEYTLS SLTSDTLYMV RMAAYTDEGG | 600 |
| KDGPEFTFTT PKFAQGEIEA IVVPVCLAFL LTTLLGVLFC FNKRDLIKKH IWPNVPDPSK | 660 |
| SHIAQWSPHT PPRHNFNSKD QMYSDGNPTD VSVVEIEAND KKPFPEDLKS LDLFKKEKIN | 720 |
| TEGHSSGIGG SSCMSSSRPS ISSSDENESS QNTSSTVQYS TVVHSGYRHQ VPSVQVFSRS | 780 |
| ESTQPLLDSE ERPEDLQLVD HVDGGDGILP RQQYFKQNCS QHESSPDISH FERSKQVSSV | 840 |
| NEEDFVRLKQ QISDHISQSC GSGQMKMFQE VSAADAFGPG TEGQVERFET VGMEAATDEG | 900 |
| MPKSYLPQTV RQGGYMPQ | |

Seq ID NO: 33 DNA sequence
Nucleic Acid Accession #: NM_018255.1
Coding sequence: 11-2491 (underlined sequences correspond to start and
stop codons)

| | |
|---|---|
| 1 11 21 31 41 51 | |
| \| \| \| \| \| \| | |
| AGTTGGCGAC ATGGTGGCAC CCGTGCTGGA GACTTCTCAC GTGTTTTGCT GCCCAAACCG | 60 |
| GGTGCGGGGA GTCCTGAACT GGAGCTCTGG GCCCAGAGGA CTTCTGGCCT TTGGCACGTC | 120 |
| CTGCTCCGTG GTGCTCTATG ACCCCCTGAA AAGGGTTGTT GTTACCAACT TGAATGGTCA | 180 |
| CACCGCCCGA GTCAATTGCA TACAGTGGAT TTGTAAACAG GATGGCTCCC CTTCTACTGA | 240 |
| ATTAGTTTCT GGAGGATCTG ATAATCAAGT GATTCACTGG GAAATAGAGG ATAATCAGCT | 300 |
| TTTAAAAGCA GTGCATCTTC AAGGCCATGA AGGACCTGTT TATGCGGTGC ATGCTGTTTA | 360 |
| CCAGAGGAGG ACATCAGATC CTGCATTATG TACACTGATC GTTTCTGCAG CTGCAGATTC | 420 |
| TGCTGTTCGA CTCTGGTCTA AAAAGGGTCC AGAAGTAATG TGCCTTCAGA CTTTAAACTT | 480 |
| TGGAAATGGA TTTGCTTTGG CTCTCTGCTT ATCTTTTTTG CCAAATACTG ATGTACCAAT | 540 |
| ATTAGCATGT GGCAATGATG ATTGCAGAAT TCACATATTT GCTCAACAAA ATGATCAGTT | 600 |
| TCAGAAAGTG CTTTCTCTCT GTGGACATGA GGATTGGATT AGAGGAGTGG AATGGGCAGC | 660 |
| CTTTGGTAGA GATCTTTTCC TAGCAAGCTG TTCACAAGAT TGCCTGATAA GAATATGGAA | 720 |
| GCTGTATATA AAGTCAACAT CTTTAGAAAC TCAGGATGAC GATAACATAA GACTGAAAGA | 780 |
| AAATACTTTT ACCATAGAAA ATGAAAGTGT TAAAATAGCA TTTGCTGTTA CTCTGGAGAC | 840 |
| AGTGCTAGCC GGTCATGAAA ACTGGGTAAA TGCAGTTCAC TGGCAACCTG TGTTTTACAA | 900 |
| AGATGGTGTC CTACAGCAGC CAGTGAGATT ATTATCTGCT TCCATGGATA AAACCATGAT | 960 |
| TCTCTGGGCT CCAGATGAAG AGTCAGGAGT TTGGCTAGAA CAGGTTCGAG TAGGTGAAGT | 1020 |
| AGGTGGGAAT ACTTTGGGAT TTTATGATTG CCAGTTCAAT GAAGATGGCT CCATGATCAT | 1080 |
| TGCTCATGCT TTCCACGGAG CGTTGCACCT TTGGAAACAG AATACAGTTA ACCCAAGAGA | 1140 |
| GTGGACTCCA GAGATTGTCA TTTCAGGACA CTTTGATGGT GTCCAAGACC TAGTCTGGGA | 1200 |
| TCCAGAAGGA GAATTTATTA TCACTGTTGG TACTGATCAG ACAACTAGAC TTTTTGCTCC | 1260 |
| ATGGAAGAGA AAAGACCAAT CACAGGTGAC TTGGCATGAA ATTGCAAGGC CTCAGATACA | 1320 |
| TGGGTATGAC CTGAAATGTT TGGCAATGAT TAATCGGTTT CAGTTTGTAT CTGGAGCAGA | 1380 |
| TGAAAAAGTT CTTCGGGTTT TTTCTGCACC TCGGAATTTT GTGGAAAATT TTTGTGCCAT | 1440 |
| TACAGGACAA TCACTGAATC ATGTGCTCTG TAATCAAGAT AGTGATCTTC CAGAAGGAGC | 1500 |
| CACTGTCCCT GCATTGGGAT TATCAAATAA AGCTGTCTTT CAGGGAGATA TAGCTTCTCA | 1560 |
| GCCTTCTGAT GAAGAGGAGC TGTTAACTAG TACTGGTTTT GAGTATCAGC AGGTGGCCTT | 1620 |
| TCAGCCCTCC ATACTTACTG AGCCTCCCAC TGAGGATCAT CTTCTGCAGA ATACTTTGTG | 1680 |
| GCCTGAAGGT CAAAAACTAT ATGGGCACGG TTATGAAATA TTTTGTGTTA CTTGTAACAG | 1740 |
| TTGAAAGACT CTGCTTGCCT CAGCTTGTAA GGCAGCTAAG AAAGAGCATG CAGCTATCAT | 1800 |
| TCTTTGGAAC ACTACATCTT GGAAACAGGT GCAGAATTTA GTTTTCCACA GTTTGACAGT | 1860 |
| CACGCAGATG GCCTTCTCAC CTAATGAGAA GTTCTTACTA GCTGTTTCCA GAGATCGAAC | 1920 |
| CTGGTCATTG TGGAAAAAGC AGGATACAAT CTCACCTGAG TTCGAGCCAG TTTTTAGTCT | 1980 |
| TTTTGCCTTC ACCAACAAAA TTACTTCTGT GCACAGTAGA ATTATTTGGT CTTGTGATTG | 2040 |
| GAGTCCTGAC AGCAAGTATT TCTTCACTGG GAGTCGAGAC AAAAAGGTGG TTGTCTGGGG | 2100 |
| TGAGTGCGAC TCCACTGATG ACTGTATTGA GCACAACATT GGCCCCTGCT CCTCAGTCCT | 2160 |
| GGACGTGGGT GGGGCTGTGA CAGCTGTCAG CGTCTGCCCA GTGCTCCACC CTTCTCAACG | 2220 |
| ATACGTGGTT GCAGTAGGAT TGGAGTGTGG AAAGATTGGC TTATATACCT GGAAAAAGAC | 2280 |
| TGATCAAGTT CCAGAAATAA ATGACTGGAC CCACTGTGTA GAAACAAGTC AAAGCCAAAG | 2340 |
| TCATACACTG GCTATCAGAA AATTATGCTG GAAGAATTGC AGTGGAAAAA CTGAACAGAA | 2400 |
| GGAAGCAGAA GGTGCTGAGT GGTTACACTT TGCAAGCTGT GGTGAAGATC ACACTGTGAA | 2460 |
| GATACACAGA GTCAATAAAT GTGCACTGTA ATGG | |

Seq ID NO: 34 Protein sequence:
Protein Accession #: NP_060725.1

| | |
|---|---|
| 1 11 21 31 41 51 | |
| \| \| \| \| \| \| | |
| MVAPVLETSH VFCCPNRVRG VLNWSSGPRG LLAFGTSCSV VLYDPLKRVV VTNLNGHTAR | 60 |
| VNCIQWICKQ DGSPSTELVS GGSDNQVIHW EIEDNQLLKA VHLQGHEGPV YAVHAVYQRR | 120 |
| TSDPALCTLI VSAAADSAVR LWSKKGPEVM CLQTLNFGNG FALALCLSFL PNTDVPILAC | 180 |
| GNDDCRIHIF AQQNDQFQKV LSLCGHEDWI RGVEWAAFGR DLFLASCSQD CLIRIWKLYI | 240 |
| KSTSLETQDD DNIRLKBNTF TIENBSVKIA FAVTLETVLA GHENWVNAVH WQPVFYKDGV | 300 |
| LQQPVRLLSA SMDKTMILWA PDEESGVWLE QVRVGEVGGN TLGFYDCQFN EDGSMIIAHA | 360 |
| FHGALHLWKQ NTVNPREWTP EIVISGHFDG VQDLVWDPEG EFIITVGTDQ TTRLFAPWKR | 420 |
| KDQSQVTWHE IARPQIHGYD LKCLAMINRF QFVSGADEKV LRVFSAPR4P VENFCAITGQ | 480 |
| SLNHVLCNQD SDLPEGATVP ALGLSNKAVF QGDIASQPSD EEELLTSTGE EYQQVAFQPS | 540 |
| ILTEPPTEDH LLQNTLWPEV QKLYGHGYEI FCVTCNSSKT LLASACKAAK KEHAAIILWN | 600 |
| TTSWKQVQNL VFHSLTVTQM AFSPNEKFLL AVSRDRTWSL WKKQDTISPE FEPVFSLFAF | 660 |
| TNKITSVHSR IIWSCDWSPD SKYFFTGSRD KKVVVWGECD STDDCIEHNI GPCSSVLDVG | 720 |
| GAVTAVSVCP VLHPSQRYVV AVGLECGKIC LYTWKKTDQV PEINDWTHCV ETSQSQSHTL | 780 |
| AIRKLCWKNC SGKTEQKEAE GAEWLIIFASC GEDHTVKIHR VNXCAL | |

Seq ID NO: 35 DNA sequence
Nucleic Acid Accession #: NM_02213.1
Coding sequence: 11-2878 (underlined sequences correspond to start and
stop codons)

| | |
|---|---|
| 1 11 21 31 41 51 | |
| \| \| \| \| \| \| | |
| TGCTGCGAGG ATGCTGCCTG GGCGGCTGTG CTGGGTGCCG CTCCTGCTGG CGCTGGGCGT | 60 |
| GGGGAGCGGC AGCGGCGGTG GCGGGGACAG CCGGCAGCGC CGCCTCCTCG CGGCTAAAGT | 120 |
| CAATAAGCAC AAGCCATGGA TCGAGACTTC ATATCATGGA GTCATAACTG AGAACAATGA | 180 |
| CACAGTCATT TTGGACCCAC CACTGGTAGC CCTGGATAAA GATGCACCGG TTCCTTTTGC | 240 |
| AGGGGAAATC TGTGCGTTCA AGATCCATGG CCAGGAGCTG CCCTTTGAGG CTGTGGTGCT | 300 |
| CAACAAGACA TCAGGAGAGG GCCGGCTCCG TGCCAAGAGC CCCATTGACT GTGAGTTGCA | 360 |
| GAAGGAGTAC ACATTCATCA TCCAGGCCTA TGACTGTGGT GCTGGGCCCC AGGAGACAGG | 420 |
| CTGGAAAAAG TCACACAAGG CCGTGGTCCA TATACAGGTG AAGGATGTCA ACGAGTTTGC | 480 |
| TCCCACCTTC AAAGAGCCAG CCTACAAGGC TGTTGTGACG GAGGGCAAGA TCTATGACAG | 540 |
| CATTCTGCAG GTGGAGGCCA TTGACGAGGA CTGCTCCCCA CAGTACAGCC AGATCTGCAA | 600 |
| CTATGAAATC GTCACCACAG ATGTGCCTTT TGCCATCGAC AGAAATGGCA ACATCAGGAA | 660 |
| CACTGAGAAG CTGAGCTATG ACAAACAACA CCAGTATGAG ATCCTGGTGA CCGCCTACGA | 720 |
| CTGTGGACAG AAGCCCGCTG CTCAGGACAC CCTGGTGCAG GTGGATGTGA AGCCAGTTTG | 780 |
| CAAGCCTGGC TGGCAAGACT GGACCAAGAG GATTGAGTAC CAGCCTGGCT CCGGGAGCAT | 840 |
| GCCCCTGTTC CCCAGCATCC ACCTGGAGAC GTGCGATGGA GCCGTGTCTT CCCTCCAGAT | 900 |
| CGTCACAGAG CTGCAGACTA ATTACATTGG GAAGGGTTGT GACCGGGAGA CCTACTCTGA | 960 |
| GAAATCCCTT CAGAAGTTAT GTGGAGCCTC CTCTGGCATC ATTGACCTCT TGCCATCCCC | 1020 |
| TAGCGCTGCC ACCAACTGGA CTGCAGGACT GCTGGTGGAC AGCAGTGAGA TGATCTTCAA | 1080 |
| GTTTGACGGC AGGCAGGGTG CCAAAATCCC CGATGGGATT GTGCCCAAGA ACCTGACCGA | 1140 |
| TCAGTTCACC ATCACCATGT GGATGAAACA CGGCCCCAGC CCTGGTGTGA GAGCCGAGAA | 1200 |
| GGAAACCATC CTCTGCAACT CAGACAAAAC CGAAATGAAC CGGCATCACT ATGCCCTGTA | 1260 |
| TGTGCACAAC TGCCGCCTCG TCTTTCTCTT GCGGAAGGAC TTCGACCAGG CTGACACCTT | 1320 |
| TCGCCCCGCG GAGTTCCACT GGAAGCTGGA TCAGATTTGT GACAAAGAGT GGCACTACTA | 1380 |
| TGTCATCAAT GTGGAGTTTC CTGTGGTAAC CTTATACATG GATGGAGCAA CATATGAACC | 1440 |
| ATACCTGGTG ACCAACGACT GGCCCATTCA TCCATCTCAC ATAGCCATGC AACTCACAGT | 1500 |
| CGGCGCTTGT TGGCAAGGAG GAGAAGTCAC CAAACCACAG TTTGCTCAGT TCTTTCATGG | 1560 |
| AAGCCTGGCC AGTCTCACCA TCCGCCCTGG CAAAATGGAA AGCCAGAAGG TGATCTCCTG | 1620 |
| CCTGCAGGCC TGCAAGGAAG GGCTGGACAT TAATTCCTfG GAAAGCCTTG GCCAAGGAAT | 1680 |
| AAAGTATCAC TTCAACCCCT CGCAGTCCAT CCTGGTGATG GAAGGTGACG ACATTGGGAA | 1740 |
| CATTAACCGT GCTCTCCAGA AAGTCTCCTA CATCAACTCC AGGCAGTTCC CAACGGCGGG | 1800 |
| TGTGCGGCGC CTCAAAGTAT CCTCCAAAGT CCAGTGCTTT GGGGAAGACG TATGCATCAG | 1860 |
| TATCCCTGAG GTAGATGCCT ATGTGATGGT CCTCCAGGCC ATCGAGCCCC GGATCACCCT | 1920 |
| CCGGGGCACA GACCACTTCT GGAGACCTGC TGCCCAGTTT GAAAGTGCCA GGGGAGTGAC | 1980 |
| CCTCTTCCCT GATATCAAGA TTGTGAGCAC CTTCGCCAAA ACCGAAGCCC CCGGGGACGT | 2040 |
| GAAAACCACA GACCCCAAAT CAGAAGTCTT AGAGGAAATG CTTCATAACT TAGATTTCTG | 2100 |
| TGACA1TITG GTGATCGGAG GGGACTTGGA CCCAAGGCAG GAGTGCTTGG AGCTCAACCA | 2160 |
| CAGTGAGCTC CACCAACGAC ACCTGGATGC CACTAATTCT ACTGCAGGCT ACTCCATCTA | 2220 |
| CGGTGTGGGC TCCATGAGCC GCTATGAGCA GGTGCTACAT CACATCCGCT ACCGCAACTG | 2280 |
| GCGTCCGGCT TCCCTTGAGG CCCGGCGTTT CCGGATTAAG TGCTCAGAAC TCAATGGGCG | 2340 |
| CTACACTAGC AATGAGTTCA ACTTGGAGGT CAGCATCCTT CATGAAGACC AAGTCTCAGA | 2400 |
| TAAGGAGCAT GTCAATCATC TGATTGTGCA GCCTCCCTTC CTCCAGTCTG TCCATCATCC | 2460 |
| TGAGTCCCGG AGTAGCATCC AGCACAGTTC AGTGGTCCCA AGCATTGCCA CAGTGGTCAT | 2520 |
| CATCATCTCC GTGTGCATGC TTGTGTTTGT CGTGGCCATG GGTGTGTACC GGGTCCGGAT | 2580 |
| CGCCCACCAG CACTTCATCC AGGAGACTGA GGCTGCCAAG GAATCTGAGA TGGACTGGGA | 2640 |
| CGATTCTGCG CTGACTATCA CAGTCAACCC CATGGAGAAA CATGAAGGAC CAGGGCATGG | 2700 |
| GGAAGATGAG ACTGAGGGAG AAGAGGAGGA AGAAGCCGAG GAAGAAATGA GCTCCAGCAG | 2760 |
| TGGCTCTGAC GACAGCGAAG AGGAGGAGGA GGAGGAAGGG ATGGGCAGAG GCAGACATGG | 2820 |
| GCAGAATGGA GCCAGGCAAG CCCAGCTGGA GTGGGATGAC TCCACCCTCC CCTACTAGTG | 2880 |
| CCCAGGGGTC TGCTGCCTGG CCCACATGTC CCTTTTGTAA ACCCTGACCC AGTGTATGCC | 2940 |
| CATGTCTATC ATACCTCACC TCTGATGTCT GTGACATGTC TGGGAAGGCC TTCTCCAGCT | 3000 |
| TCCTGGAGCC CACCCTTTAA GCCTTGGGCA CTCCCTGTGT TTCATCCATG GGGAAGTTCC | 3060 |
| AAGAAGCCCA GCATGGCCAT CAGTGAGGAC TTCAGGGTAG ACTTTGTCCT GTAGCCTCCA | 3120 |
| CTTCTGCCCT AAGTTCCCCA GCATCCTGAC TACCTGTCTG CAGAGTTTGC CTTTGTTTTT | 3180 |
| TCCTGCAGGG AAGAAGGCCC ACCTTTGTGT CACTCACCTC CCCAGGCTCA GAGTCCCCAA | 3240 |
| GGCCCTGGGG TTCCAACTCA CTGTGCGTCT CCTCCACACA GACCAGTAGG TTCTCCTATG | 3300 |
| CTGACTCCAG GGTGCTTCAT ACAAGGAGGG TGGTTGAACT TCACACACGT AAGGTCTTAG | 3360 |
| TGCTTAACAG GTTAAAGGAA AGTCCTTGTT GAGGCAGAAC TAAGTTTACA GGGAAAGGTA | 3420 |
| CACACATTCT CTCTCTCTCT CTCTCTCTGT CTATCTAGTT CCCCAGCTTG GAGAGCCTTT | 3480 |
| CCCCTTGCTT CTTTCTGAGG CCATATAAGC TTATAAGAAA AGTCCCAAAC CAAGAATAGG | 3540 |
| TCCTTGGCCA CAAGCAGGGT CTGATCCCCC ATCAGAGCTA TCTGAGCCTG CCTGTCTGGG | 3600 |
| CACCTGCTGC AACCATGCAG CTACCCTGCC AGGGGCACTC AGCAAACAGA ACCACAGGGC | 3660 |
| CCAGGAGGCA GTCCACACAG GCACTGCCCC AGGACAACAC AACAAGGACA GTCACAACAA | 3720 |
| GGACAACAAG GACACAACAC AACACACAAC AAGGACAGTC ACAACAAGCC TAGAGCCAGA | 3780 |
| AAGCAGATGG AAATGCTAAT GAGGTCAAAC GTAGGCTTCA TGGTGGGTGG AGTGGGGGTG | 3840 |
| GCTGGGCTCC CCCAGGACAG AGGGGACCCT GAGGTTGGCA AGGCTCTCAC CACTCAGCCT | 3900 |
| TATGGTCCCT TATCTCCTAT CTTCCCTCTT GAGAAAATAC ACGCTTTCTG CATGTATTAG | 3960 |
| AAACGCACGA GCTCCACCAA GTCTACAATG AAAGTTTGAA ATTTAACTGC AAGGAATTAG | 4020 |
| AAGCATATTT GCAATCATTG CAGCTTCTTC TTTCTTCTGC TCATAAAAGG AGGAACACTT | 4080 |
| TAGATAGAGG GCAAATATAT CTGAAAACCT AATTTCTTTC TTTTTTTGAT AAGGAAATCT | 4140 |
| TTTCCATCTC CATCCTAACA TGCACAACCT GTGAAGAGAA TTGTTTCTAT AGTAACTGGT | 4200 |
| CTGTGATCTT TTGTGGCCAA GAGAATAGCA GGCAAGAATT AGGGCCTTGA CAGAATGTCC | 4260 |
| ACGAAGCTCT GAGAACATGT TTGTTTCGAA TGTCTGATTC CTCTTTGTCA TCAATGTGTA | 4320 |
| TGCTCTGTCC CCATCCTTCA CTCCTCCTCA AGCTCACACC AATTGGTTTG GCACAGGCAC | 4380 |
| AGAGCTGGTC CCTAGTTAAG TGGCATTTAT GTTAAAAAAA AATAGTTCAG AATCTCAGCC | 4440 |
| TTTTCTTTGT GTCATCAAAA CAGCTTAAGA AGGGGACTAC TGCCAATGTC CTCTAGTCTG | 4500 |
| ACCTCCACCC AGGGAGGACC CATGGCAGGT CTTTTCAACT TTCTGATTCA TGAGAACAAC | 4560 |
| CTTGTGAAGC TTTTCCCACC TCCTAAAGTG TTTTCTGCAT CTGTTCCTTC CTTTGGACCT | 4620 |
| CACAACAAAT CCTGTGAAGT AACTGAGACA TCTGTTGTTA GATACATTTT TGTGATGAGT | 4680 |
| AAACTGAGGC TTCG | |
| Seq ID NO: 36 Protein sequence: | |
| Protein Accession #: NP_071414.1 | |
| 1 11 21 31 41 51 | |
| \| \| \| \| \| \| | |
| MLPGRLCWVP LLLALGVGSG SGGGGDSRQR RLLAAKVNKH KPWIETSYHG VITENNDTVI | 60 |
| LDPPLVALDK DAPVPEAGEI CAFKIHGQEL PFEAVVLNKT SGEGRLRAKS PIDCELQKEY | 120 |
| TFIIQAYDCG AGPHBTAWKK SHKAVVHIQV KDVNEFAPTF KEPAYKAVVT EGKIYDSILQ | 180 |
| VEAIDEDCSP QYSQICNYEI VTTDVPFAID RNGNIRNTEK LSYDKQHQYE ILVTAYDCGQ | 240 |
| KPAAQDTLVQ VDVKPVCKPG WQDWTKRIEY QPGSGSMPLF PSIHLETCDG AVSSLQIVTE | 300 |
| LQTNYIGKGC DRETYSEKSL QKLCGASSGI IDLLPSPSAA TNWTAGLLVD SSEMIEKFDG | 360 |
| RQGAKIPDGI VPKNLTDQFT ITMWMKHGPS PGVRAEKETI LCNSDKTEMN RHHYALYVHN | 420 |
| CRLVFLLRXD FDQADTERPA EFHWKLDQIC DKEWHYYVIN VEFPVVTLYM DGATYEPYLV | 480 |
| TNDWPIHPSH IAMQLTVGAC WQGGEVTKPQ EAQFFHGSLA SLTIRPGKME SQKVISCLQA | 540 |
| CKEGLDINSL ESLGQGIKYH ENPSQSILVM EGDDIGNTNR ALQKVSYINS RQFPTAGVRR | 600 |
| LKVSSKVQCF GEDVCISIPE VDAYVMVLQA IEPRITLRGT DHFWRPAAQF ESARGVTLFP | 660 |
| DIKIVSTFAK TEAPGDVKTT DPKSEVLEEM LHNLDFCDIL VIGGDLDPRQ ECLELNHSEL | 720 |
| HQRHLDATNS TAGYSIYGVG SMSRYEQVLH HIRYRNWRPA SLEARRFRIK CSELNGRYTS | 780 |
| NBFNLEVS1L HEDQVSDKEH VNHLIVQPPF LQSVHHPESR SSIQHSSVVP SIATVVIIIS | 840 |
| VCMLVFVVAM GVYRVRIAHQ HFIQETEAAX ESEMDWDDSA LTITVNPMEK HEGPGHGEDE | 900 |
| TEGEEEEEAE EEMSSSSGSD DSEEEEEEEG MGRGRHGQNG ARQAQLEWDD STLPY | |

Seq ID NO: 37 DNA sequence
Nucleic Acid Accession #: none found
Coding sequence: 143-874 (underlined sequences correspond to start and
stop codons)

| | |
|---|---|
| 1 11 21 31 41 51 | |
| \| \| \| \| \| \| | |
| GGGAGGGAGA GAGGCGCGCG GGTGAAAGGC GCATTGATGC AGCCTGCGGC GGCCTCGGAG | 60 |
| CGCGGCGGAG CCAGACGCTG ACCACGTTCC TCTCCTCGGT CTCCTCCGCC TCCAGCTCCG | 120 |
| CGCTGCCCGG CAGCCGGGAG CCATGCGACC CCAGGGCCCC GCCGCCTCCC CGCAGCGGCT | 180 |
| CCGCGGCCTC CTGCTGCTCC TGCTGCTGCA GCTGCCCGCG CCGTCGAGCG CCTCTGAGAT | 240 |
| CCCCAAGGGG AAGCAAAAGG CGCAGCTCCG GCAGAGGGAG GTGGTGGACC TGTATAATGG | 300 |
| AATGTGCTTA CAAGGGCCAG CAGGAGTGCC TGGTCGAGAC GGGAGCCCTG GGGCCAATGG | 360 |
| CATTCCGGGT ACACCTGGGA TCCCAGGTCG GGATGGATTC AAAGGAGAAA AGGGGGAATG | 420 |
| TCTGAGGGAA AGCTTTGAGG AGTCCTGGAC ACCCAACTAC AAGCAGTGTT CATGGAGTTC | 480 |
| ATTGAATTAT GGCATAGATC TTGGGAAAAT TGCGGAGTGT ACATTTACAA AGATGCGTTC | 540 |
| AAATAGTGCT CTAAGAGTTT TGTTCAGTGG CTCACTTCGG CTAAAATGCA GAAATGCATG | 600 |
| CTGTCAGCGT TGGTATTTCA CATTCAATGG AGCTGAATGT TCAGGACCTC TTCCCATTGA | 660 |
| AGCTATAATT TATTTGGACC AAGGAAGCCC TGAAATGAAT TCAACAATTA ATATTCATCG | 720 |
| CACTTCTTCT GTGGAAGGAC TTTGTGAAGG AATTGGTGCT GGATTAGTGG ATGTTGCTAT | 780 |
| CTGGGITGGC ACTTGTTCAG ATTACCCAAA AGGAGATGCT TCTACTGGAT GGAATTCAGT | 840 |
| TTCTCGCATC ATTATTGAAG AACTACCAAA ATAAATGCTT TAATTTTCAT TTGCTACCTC | 900 |
| TTTTTTTATT ATGCCTTGGA ATGGTTCACT TAAATGACAT TTTAAATAAG TTTATGTATA | 960 |
| CATCTGAATG AAAAGCAAAG CTAAATATGT TTACAGACCA AAGTGTGATT TCACACTGTT | 1020 |
| TTTAAATCTA GCATTATTCA TTTTGCTTCA ATCAAAAGTG GTTTCAATAT TTTTTTTAGT | 1080 |
| TGGTTAGAAT ACTTTCTTCA TAGTCACATT CTCTCAACCT ATAATTTGGA ATATTGTTGT | 1140 |
| GGTCTITTGT TTTTTCTCTT AGTATAGCAT TTTTAAAAAA ATATAAAAGC TACCAATCTT | 1200 |
| TGTACAATTT GTAAATG1TA AGAATTTTTT TTATATCTGT TAAATAAAAA TTATTTCCAA | 1260 |
| CAACCTTAAA AAAAAAAAAA AAAA | |

Seq ID NO: 38 Protein sequence:
Protein Accession #: none found

| | |
|---|---|
| 1 11 21 31 41 51 | |
| \| \| \| \| \| \| | |
| MRPQGPAASP QRLRGLLLLL LLQLPAPSSA SEIPKGKQKA QLRQREVVDL YNGMCLQGPA | 60 |
| GVPGRDGSPG ANGIPGTPGI PGRDGFKGEK GECLRESFEE SWTPNYKQCS WSSLNYGIDL | 120 |
| GKIAECTFTK MRSNSALRVL FSGSLRLKCR NACCQRWYFT FNGAECSGPL PIEAI1YLDQ | 180 |
| GSPEMNSTIN IHRTSSVEGL CEGIGAGLVD VA1WVGTCSD YPKGDASTGW NSVSRIIIEE | 240 |
| LPK | |

Seq ID NO: 39 DNA sequence
Nucleic Acid Accession #: NM_000949
Coding sequence: 285-2153 (underlined sequences correspond to start
and stop codons)

| | |
|---|---|
| 1 11 21 31 41 51 | |
| \| \| \| \| \| \| | |
| GGAGGCTGAA ATCCCCAGAC GCCGGTTTTC TGGGCTGGGC TTTCTGCTTA CTCACTCCTT | 60 |
| CTCCCTCTTT CTGGATTTTA CCGACCGTTC GCGAAACAGC TTTCCACACA ATGGAGCTTC | 120 |
| ATGTCCTCGT GCAGGAAGTA CTCATCGACT GATGTGGCAG ACTTTGCTCC CTGACAAAAC | 180 |
| TAAAGAACTC TCCTATTCAT GGAGGCGAAC ACTGAGGATG CTTTCCACAT GAACCCTGAA | 240 |
| GTGAACTTCT GATACATTTC CTGCAGCAAG AGAAGGCAGC CAACATGAAG GAAAATGTGG | 300 |
| CATCTGCAAC CGTTTTCACT CTGCTACTTT TTCTCAACAC CTGCCTTCTG AATGGACAGT | 360 |
| TACCTCCTGG AAAACCTGAG ATCTTTAAAT GTCGTTCTCC CAATAAGGAA ACATTCACCT | 420 |
| GCTGGTGGAG GCCTGGGACA GATGGAGGAC TTCCTACCAA TTATTCACTG ACTTACCACA | 480 |
| GGGAAGGAGA GACACTCATG CATGAATGTC CAGACTACAT AACCGGTGGC CCCAACTCCT | 540 |
| GCCACTTTGG CAAGCAGTAC ACCTCCATGT GGAGGACATA CATCATGATG GTCAATGCCA | 600 |
| CTAACCAGAT GGGAAGCAGT TTCTCGGATG AACTTTATTT GGACGTGACT TACATAGTTC | 660 |
| AGCCAGACCC TCCTTTGGAG CTGGCTGTGG AAGTAAAACA GCCAGAAGAC AGAAAACCCT | 720 |
| ACCTGTGGAT TAAATGGTCT CCACCTACCC TGATTGACTT AAAAACTGGT TGGTTCACGC | 780 |
| TCCTGTATGA AATTCGATTA AAACCCGAGA AAGCAGCTGA GTGGGAGATC CATTTTGCTG | 840 |
| GGCAGCAAAC AGAGTTTAAG ATTCTCAGCC TACATCCAGG ACAGAAATAC CTTGTCCAGG | 900 |
| TTCGCTGCAA ACCAGACCAT GGATACTGGA GTGCATGGAG TCCAGCGACC TTCATfCAGA | 960 |
| TACCTAGTGA CTTCACCATG AATGATACAA CCGTGTGGAT CTCTGTGGCT GTCCTTTCTG | 1020 |
| CTGTCATCTG TTTGATTATT GTCTGGGCAG TGGCTTTGAA GGGCTATAGC ATGGTGACCT | 1080 |
| GCATCTTTCC GCCAGTTCCT GGGCCAAAAA TAAAAGGATT TGATGCTCAT CTGTTGGAGA | 1140 |
| AGGGCAAGTC TGAAGAACTA CTGAGTGCCT TGGGATGCCA AGACTTTCCT CCCACTTCTG | 1200 |
| ACTATGAGGA CTTGCTGGTG GAGTAT1TAG AAGTAGATGA TAGTGAGGAC CAGCATCTAA | 1260 |
| TGTCAGTCCA TTCAAAAGAA CACCCAAGTC AAGGTATGAA ACCCACATAC CTGGATCCTG | 1320 |
| ACACTGACTC AGGCCGGGGG AGCTGTGACA GCCCTTCCCT TTTGTCTGAA AAGTGTGAGG | 1380 |
| AACCCCAGGC CAATCCCTCC ACATTCTATG ATCCTGAGGT CATTGAGAAG CCAGAGAATC | 1440 |
| CTGAAACAAC CCACACCTGG GACCCCCAGT GCATAAGCAT GGAAGGCAAA ATCCCCTATT | 1500 |
| TTCATGCTGG TGGATCCAAA TGTTCAACAT GGCCCTTACC ACAGCCCAGC CAGCACAACC | 1560 |
| CCAGATCCTC TTACCACAAT ATTACTGATG TGTGTGAGCT GGCTGTGGGC CCTGCAGGTG | 1620 |
| CACCGGCCAC TCTGTTGAAT GAAGCAGGTA AAGATGCTTT AAAATCCTCT CAAACCATTA | 1680 |
| AGTCTAGAGA AGAGGGAAAG GCAACCCAGC AGAGGGAGGT AGAAAGCTTC CATTCTGAGA | 1740 |
| CTGACCAGGA TACGCCCTGG CTGCTGCCCC AGGAGAAAAC CCCCTTTGGC TCCGCTAAAC | 1800 |
| CCTTGGATTA TGTGGAGATT CACAAGGTCA ACAAAGATGG TGCATTATCA TTGCTACCAA | 1860 |
| AACAGAGAGA GAACAGCGGC AAGCCCAAGA AGCCCGGGAC TCCTGAGAAC AATAAGGAGT | 1920 |
| ATGCCAAGGT GTCCGGGGTC ATGGATAACA ACATCCTGGT GTTGGTGCCA GATCCACATG | 1980 |
| CTAAAAACGT GGCTTGCTTT GAAGAATCAG CCAAAGAGGC CCCACCATCA CTTGAACAGA | 2040 |
| ATCAAGCTGA GAAAGCCCTG GCCAACTTCA CTGCAACATC AAGCAAGTGC AGGCTCCAGC | 2100 |
| TGGGTGGTTT GGATTACCTG GATCCCGCAT GTTTTACACA CTCCTTTCAC TGATAGCTTG | 2160 |
| ACTAATGGAA TGATTGGTTA AAATGTGATT TTTCTTCAGG TAACACTACA GAGTACGTGA | 2220 |
| AATGCTCAAG AATGTAGTCA GACTGACACT ACTAAAGCTC CCAGCTCCTT TCATGCTCCA | 2280 |
| TTTTTAACCA CTTGCCTCTT TCTCCAGCAG CTGATTCCAG AACAAATCAT TATGTTTCCT | 2340 |
| AACTGTGATT TGTAGATTTA CTTTTTGCTG TTAGTTATAA AACTATGTGT TCAATGAAAT | 2400 |
| AAAAGCACAC TGCTTAGTAT TCTTGAGGGA CAATGCCAAT AGGTATATCC TCTGGAAAAG | 2460 |
| GCTTTCATGA TTTGGCATGG GACAGACGGA AATGAAATTG TCAAAATTGT TTACCATAGA | 2520 |
| AAGATGACAA AAGAAAATTT TCCACATAGG AAAATGCCAT GAAAATTGCT TTTGAAAAAC | 2580 |
| AACTGCATAA CCTFFACACT CCTCGTCCAT TTTATTAGGA TTACCCAAAT ATAACCATTT | 2640 |
| AAAGAAAGAA TGCATTCCAG AACAAATTGT TTACATAAGT TCCTATACCT TACTGACACA | 2700 |
| TTGCTGATAT GCAAGTAAGA AAT | |

Seq ID NO: 40 Protein sequence:
Protein Accession #: NP_000940.1

| | |
|---|---|
| 1 11 21 31 41 51 | |
| \| \| \| \| \| \| | |
| MKENVASATV FTLLLFLNTC LLNGQLPPGK PEIFKCRSPN KETFTCWWRP GTDGGLPTNY | 60 |
| SLTYHREGET LMHECPDYIT GGPNSCHFGK QYTSMWRTYI MMVNATNQMG SSFSDELYVD | 120 |
| VTYIVQPDPP LELAVEVKQP EDRKPYLWIK WSPPTLIDLK TGWFTLLYEI RLKPEKAAEW | 180 |
| EIHFAGQQTE FKILSLHPGQ KYLVQVRCKP DHGYWSAWSP ATPIQIPSDF TMNDTTVWIS | 240 |
| VAVLSAVICL IIVWAVALKG YSMVTCIFPP VPGPKJKGFD AHLLEKGKSE ELLSALGCQD | 300 |
| FPPTSDYEDL LVEYLEVDDS EDQHLMSVHS KEHPSQGMKP TYLDPDTDSG RGSCDSPSLL | 360 |
| SEKCEEPQAN PSTEYDPEVI EKPENPETTH TWDPQCISME GKIPYFHAGG SKCSTWPLPQ | 420 |
| PSQHNPRSSY HNITDVCELA VGPAGAPATL LNEAGKDALK SSQTIKSREE GKATQQREVE | 480 |
| SFHSETDQDT PWLLPQEKTP FGSAKPLDYV EIHXVNXDGA LSLLPKQREN SGKPKKPGTP | 540 |
| ENNXEYAKVS GVMDNNILVL VPDPHAKNVA CFEESAKEAP PSLEQNQAEK ALMIFTATSS | 600 |
| KCRLQLGGLD YLDPACPTHS FH | |

Seq ID NO: 41 DNA sequence
Nucleic Acid Accession #: none found, Eos cloned sequence
Coding sequence: 1-1572 (underlined sequences correspond to start and
stop codons)

| | |
|---|---|
| 1 11 21 31 41 51 | |
| \| \| \| \| \| \| | |
| ATGACCCAAA ATAAATTAAA GCTTTGTTCC AAAGCCAATG TGTATACTGA AGTGCCTGAT | 60 |
| GGAGGATGGG GCTGGGCGGT AGCTGTTTCA TTTTTCTTCG TTGAAGTCTT CACCTACGGC | 120 |
| ATCATCAAGA CATTTGGTGT CTTCTTTAAT GACTTAATGG ACAGTTTTAA TGAATCCAAT | 180 |
| AGCAGGATCT CATGGATAAT CTCAATCTGT GTGTTTGTCT TAACATITTC AGCTCCCCTC | 240 |
| GCCACAGTCC TGAGCAATCG TTTCGGACAC CGTCTGGTAG TGATGTTGGG GGGGCTACTT | 300 |
| GTCAGCACCG GGATGGTGGC CGCCTCCTTC TCACAAGAGG TTTCTCATAT GTACGTCGCC | 360 |
| ATCGGCATCA TCTCTGGTCT GGGATACTGC TTTAGTTTTC TCCCAACTGT AACCATCCTA | 420 |
| TCACAATATT TTGGCAAAAG ACGTTCCATA GTCACTGCAG TTGCTTCCAC AGGAGAATGT | 480 |
| TTCGCTGTGT TTGCTTTCGC ACCAGCAATC ATGGCTCTGA AGGAGCGCAT TGGCTGGAGA | 540 |
| TACAGCCTCC TCTTCGTGGG CCTACTACAG TTAAACATTG TCATCTTCGG AGCACTGCTC | 600 |
| AGACCCATCT TTATCAGAGG ACCAGCGTCA CCGAAAATAG TCATCCAGGA AAATCGGAAA | 660 |
| GAAGCGCAGT ATATGCTTGA AAATGAGAAA ACACGAACCT CAATAGACTC CATTGACTCA | 720 |
| GGAGTAGAAC TAACTACCTC ACCTAAAAAT GTGCCTACTC ACACTAACCT GGAACTGGAG | 780 |
| CCGAAGGCCG ACATGCAGCA GGTCCTGGTG AAGACCAGCC CCAGGCCAAG CGAAAAGAAA | 840 |
| GCCCCGCTAT TAGACTTCTC CATTITGAAA GAGAAAAGTT TTATTTGTTA TGCATTATTT | 900 |
| GGTCTCTTTG CAACACTGGG ATTCTTTGCA CCTTCCTTGT ACATCATTCC TCTGGGCATT | 960 |
| AGTCTGGGCA TTGACCAGGA CCGCGCTGCT TTTTTATTAT CTACGATGGC CATTGCAGAA | 1020 |
| GTTTTCGGAA GGATCGGAGC TGGTTTTGTC CTCAACAGGG AGCCCATTCG TAAGATTTAC | 1080 |
| ATTGAGCTCA TCTGCGTCAT CTTATTGACT GTGTCTCTGT TTGCCTTTAC TTTTGCTACT | 1140 |
| GAATTCTGGG GTCTAATGTC ATGCAGCATA TTTTTTGGGT TTATGGTTGG AACAATAGGA | 1200 |
| GGGACTCACA TTCCACTGCT TGCTGAGGAT GATGTCGTGG GCATTGAGAA GATGTCTTCT | 1260 |
| GCAGCTGGGG TCTACATCTT CATTCAGAGC ATAGCAGGAC TGGCTGGACC GCCCCTTGCA | 1320 |
| GGTTTGTTGG TGGACCAAAG TAAGATCTAC AGCAGGGCCT TCTACTCCTG CGCAGCTGGC | 1380 |
| ATGGCCCTGG CTGCTGTGTG CCTCGCCCTG GTGAGACCGT GTAAGATGGG ACTGTGCCAG | 1440 |
| CATCATCACT CAGGTGAAAC AAAGGTAGTG AGCCATCGTG GGAAGACTTT ACAGGACATA | 1500 |
| CCTGAAGACT TTCTGGAAAT GGATCTTGCA AAAAATGAGC ACAGAGTTCA CGTGCAAATG | 1560 |
| GAGCCGGTAT GA | |

Seq ID NO: 42 Protein sequence:
Protein Accession #: none found, Eos cloned sequence

| | |
|---|---|
| 1 11 21 31 41 51 | |
| \| \| \| \| \| \| | |
| MTQNKLKLCS KANVYTEVPD GGWGWAVAVS FFFVEVFTYG IIKTFGVFFN DLMDSFNBSN | 60 |
| SRISWIISIC VFVLTFSAPL ATVLSNRFGH RLVVMLGGLL VSTGMVAASF SQEVSHMYVA | 120 |
| IGIISGLGYC FSFLPTVTIL SQYFGKRRSI VTAVASTGEC FAVFAFAPAI MALKERIGWR | 180 |
| YSLLFVGLLQ LNIVIFGALL RPIFIRGPAS PKIVIQENRX EAQYMLENEK TRTSIDSIDS | 240 |
| GVELTTSPKN VPTHTNLELE PKADMQQVLV KTSPRPSEKK APLLDFSILK EKSFICYALF | 300 |
| GLFATLGFFA PSLYIIPLGI SLGIDQDRAA FLLSTMAIAE VPGRIGAGFV LNREPIRK1Y | 360 |
| IELICVILLT VSLFAETFAT EFWGLMSCSI FFGFMVGTIG GTHIPLLAED DVVGIEKMSS | 420 |
| AAGVYIFIQS IAGLAGPPLA GLLVDQSKIY SRAFYSCAAG MALAAVCLAL VRPCKMGLCQ | 480 |
| HHHSGETKVV SHRGKTLQDI PEDFLEMDLA KNEHRVHVQM EPV | |

Seq ID NO: 43 DNA sequence
Nucleic Acid Accession #: FGENESH predicted ORF
Coding sequence: 1-1749 (underlined sequences correspond to start and
stop codons)

| | |
|---|---|
| 1 11 21 31 41 51 | |
| \| \| \| \| \| \| | |
| ATGCTGTCTG GCTTCTTGAT GAGTCCCAGT ACCCAGCACA GAGCACAGTA CACTCCCGGA | 60 |
| GGAAAGAAAC TTCCGTGGGA GGCTTCCATC GGTGCGCACA CCTCCCGAGG GCGAGGCAGC | 120 |
| GACCGGGAGA GGGAGAGCCG GCCGGAGGCT GCCGGGCTCC TGTGGGACCG CGCTGCAGCC | 180 |
| GGGGAGGCGG AGAAGGGGAA CCGGGGCGAG CCGCCCGCCT GGATCCGCGC CCAGCAGCAG | 240 |
| CCGCGGCCGC CGCCAGCTGG GCAGGCTCCC GGGACTGCGG CTGGGGGCGC GCAGGACCCT | 300 |
| CGCCTGCGTC CTGGACGTTC CCGGGGGAGG GTCCGGTTGC CAGTGAAACC TCCAGAGGCT | 360 |
| TCCGGACGAC AGCCCCGGGG GCCTTCTGAC TGCATCCCGA GATTTCCATC AGCGAGTGCA | 420 |
| ACTCATAAGG CAGTCCCTAA GGGGACCGGG CCACCGGCTG AGGACGGGGA TGGCTTAGGA | 480 |
| GCTCCTGGAC CTAGGGCCCG GCGTCGTCGC CTCCTGGGCG TCGCGGCAGA GGGGAGTGGC | 540 |
| CCGCGCGGAA AGCGCCGCGG GACAGTCAGT GACGAGGCCC GGGGGTCGCC GGGGCCACGA | 600 |
| CTTCTCGGAG ACCGTCCTGC GCTCTCTGGA GACGCGCTGT CCGCGCCCAG GGTGGTGCCA | 660 |
| TGTGGGGCGC TCGCCGCTCG TCCGTCTCCT CATCCTGGAA CGCCGCTTCG CTCCTGCAGC | 720 |
| TGCTGCTGGC TGCGCTGCTG GCGGCGGGGG CGAGGGCCCA GCGGCGAGTA CTGCCACGGC | 780 |
| TGGCTGGACG CGCAGGGCGT CTGGCGCATC GGCTTCCAGT GTCCCGAGCG CTTCGACGGC | 840 |
| GGCGACGCCA CCATCTGCTG CGGCAGCTGC GCGTTGCGCT ACTGCTGCTC CAGCGCCGAG | 900 |
| GCGCGCCTGG ACCAGGGCGG CTGCGACAAT GACCGCCAGC AGGGCGCTGG CGAGCCTGGC | 960 |
| CGGGCGGACA AAGACGGGCC CCGACGGCTC GGCAGGGCTT CATGTCTTAG GGGTACCCAA | 1020 |
| GGAGACGGCG AGGGTGCGCC CCCACCCGTG AGGGCCTGGC AGCGGTGCTC CCCTGAAGGC | 1080 |
| TCCCCGAAAG GAAGGCAGCT CCTCAGGGCT TTCCCGGGGC TGCTGCCCCG TGCCAGACGC | 1140 |
| CGCGGATTCC CATCTTCTCC ACGCGGCGGC CCCTCTCCCC TGCAGCGGCC CGCCTTGCCC | 1200 |
| ATCTACGTGC CGTTCCTCAT TGTTGGCTCC GTGTTTGTCG CCTTTATCAT CTTGGGGTCC | 1260 |
| CTGGTGGCAG CCTGTTGCTG CAGATGTCTC CGGCCTAAGC AGGATCCCCA GCAGAGCCGA | 1320 |
| GCCCCAGGGG GTAACCGCTT GATGGAGACC ATCCCCATGA TCCCCAGTGC CAGCACCTCC | 1380 |
| CGGGGGTCGT CCTCACGCCA GTCCAGCACA GCTGCCAGTT CCAGCTCCAG CGCCAACTCC | 1440 |
| GGGGCCCGGG CGCCCCCAAC AAGGTCACAG ACCAACTGTT GCTTGCCGGA AGGGACCATG | 1500 |
| AACAACCTGT ATGTCAACAT GCCCACGAAT TTCTCTGTGC TGAACTGTCA GCAGGCCACC | 1560 |
| CAGATTGTGC CACATCAAGG GCAGTATCTG CATCCCCCAT ACGTGGGGTA GACGGTGCAG | 1620 |
| CACGACTCTG TGCCCATGAC AGGTGTGCCA CCTTTCATGG ACGGCCTGCA GCCTGGCTAC | 1680 |
| AGGCAGATTC AGTCCCCCTT CCCTCACACC AACAGTGAAC AGAAGATGTA CCCAGCGGTG | 1740 |
| ACTGTATAAC CGAGAGTCAC TGGTGGGTTC CTTTACTGAA GGGAGACGAA GGCAGGGGTG | 1800 |
| GATTCTCGAG GTGGAAGTCC GCACATGTCG GTGGTATTTA TGGCACGATT CGTTTGGATG | 1860 |
| GCTTCATTTG CCCCCAGACT GTATGAAAAC ATCTCCGAAT TAGCATTTCT GGATATGTTT | 1920 |
| CATCCAGGGT ATCATTGATT TATGATGGAA AACCGGCCTC AGCTGGAGAT GACTGTGATG | 1980 |
| TTGCTGATGG GTGTATAACA AATGCTTGAG TCCGAAGTGC CCTTGAGATA TGGTTGACGA | 2040 |
| AAGAATTTTA TAAACTGATA AATTAAGGAT TTTTATTATG TTGTTATTAT TATTTCTTTT | 2100 |
| TTGTTGTTGA CTGCACAGGA TCAAAATGCC TGTTATCTCC CTTTTACTGG GACTTTTTTT | 2160 |
| TTTTTTTTTT TTTTTTTTAA TCAGACAGGG TCTTGCTCTG TTGCCCAGGC TGGAGTGCAG | 2220 |
| TGGTGCGATC TCGGCTCACT GCAACTTCAG CCTCCTGGAT TCAGGCAACA CTCCTGCCTC | 2280 |
| AGCCTCCCAC GTGGCTGGGA TTACAGGTGC CTGCCCCCAT GGCTAATTTT TTGTATTTTT | 2340 |
| TGTAGAGATG GGGTTTCACC ATGTTGGCTG CCCTGGTCTC ACTCTCCTGA CCTCAAGCAA | 2400 |
| TCTGCCTGTC TCAGCCTCCC AAAGTGCTGG GATTACAGGC GTGAGCCACC GCCCCCAGCC | 2460 |
| TGAGCCTTTT TTTITITCTA ATGCATCCAA GGTTAAGGGG AAGACGCAAA TAACAGGACT | 2520 |
| ATTCTAAAAG GAAACCTGTT TGAACTCTGT GAGATCAGTC ATCAGTCTCA GTATTCCACA | 2580 |
| GGCACACCTT AATTTCAITG TAAAAAGATA TATATATTTT GTCTATTTTT GTGCTTTTGG | 2640 |
| GGGCCTATIT TGTGCTTTTT TACCTTATGT AGAGATCTTA TTACAAAGTG ATTTTCTACA | 2700 |
| TTAAAAAGAG ACTGAAATAA ATTGTATAGT TACTTAACTA ATGAAGACAT TTCAGAACTC | 2760 |
| TGGGATGATT TTAATCTTGA AGTAGTAGGT GGTATAGTCA TAAAACCATT CATCCCCTTC | 2820 |
| TTGATTGTAT CTTAATTTTC TGGCTTTAAG GTGACATCTG AGAGGTAATG CATTCTTTTT | 2880 |
| TATATTGAAA TCATAAACTA TCACCCGCTG CTTCTCTGAG TTACTTTTAA TTTTGCCTTG | 2940 |
| TGGTTATGGT TTGGCGTTTC CTTCTGTTTG GTTTTCAGAG CCCCATGTCT ATATAGTCCT | 3000 |
| GAGTGCAAGT AATTACTATA GTTQTAAATG AAGATCAGTA TTTCTGCCTA GATCTGATAA | 3060 |
| AAAAATTTTC TTGTCTTAGT TATAAAAATT CAAAGAAATG TGTTACAAAG ATACTTAGTA | 3120 |
| TAGCTCCTCA GCCATAACCT GAGACTTGGG ATGAAATTTA AACCAGATAC GATTTACTTT | 3180 |
| GCAGATCATA AGGCTTTTTA TACTCTTGTT ATCAAAATGG CTTATTTTTC AGGCACTAAG | 3240 |
| GATTGTTAAG AGAAAAGCTT TTCAACGAAG GATTGCCTTT CTTCTCCCAC ACTGTTCTTG | 3300 |
| ATTTCCTCTC TCTTTCAGGC CTCAACAGGC ACTGTATTCA TTGCCAATGT TCCAAATTAT | 3360 |
| CAAATTCAAG TGAATTTATT TGTGTGTTCT TTACTTATAT AAAAAAAGAT AACTTTAAGG | 3420 |
| ATGTGCAAGT ACATTTCCAA CTGCTAGCAC AACCAGTATT TGTAATTTAA ACAAATCGCT | 3480 |
| GTATGGTATG GTCTTCTACA CATTTATGTC TATAGATATC TATCGATCAT CTTTCTATTC | 3540 |
| TGTTTCATGA CTGAATAATG TAAAACCAGT GTTGGCAATT GGTATCATCA ATGATACTCA | 3600 |
| TTTTTTAATA ACCAAAGGCA GGGGAAAATC ATTTTACTTA TTAATAAATA TTTTATGATG | 3660 |
| TGAAAAAAAA AAAAAAAAAA AAAAAAAAAA | |

Seq ID NO: 44 Protein sequence:
Protein Accession #: FGENESH predicted

| | |
|---|---|
| 1 11 21 31 41 51 | |
| \| \| \| \| \| \| | |
| MLSGFLMSPS TQHRAQYTPG GKXLPWEASI GAHTSRGRGS DRBRESRPEA AGLLWDRAAA | 60 |
| GEAEKGNRGE PPAWIRAQQQ PRPPPAGQAP GTAAGGAQDP RLRPGRSRGR VRLPVKPPEA | 120 |
| SGRQPRGPSD CIPREPSASA THKAVPKGTG PPAEDGDGLG APGPRARRRR LLGVAAEGSG | 180 |
| PRGKRRGTVS DEARGSPGPR LLGDRPALSG DALSAPRVVP CGALAARPSP HPGTPLRSCS | 240 |
| CCWLRCWRRG RGPSGEYCHG WLDAQGVWRI GEQCPERFDG GDATICCGSC ALRYCCSSAE | 300 |
| ARLDQGGCDN DRQQGAGEPG RADKDGPRRL GRASCLRGTQ GDGEGAPPPV RAWQRCSPEG | 360 |
| SPKGRQLLRA FPGLLPRARR RGFPSSPRGG PSPLQRPALP IYVPFLIVGS VFVAFIILGS | 420 |
| LVAACCCRCL RPKQDPQQSR APGGNRLMET IPMIPSASTS RGSSSRQSST AASSSSSANS | 480 |
| GARAPPTRSQ TNCCLPEGTM NNVYVNMPTN FSVLNCQQAT QIVPHQGQYL HPPYVGYTVQ | 540 |
| HDSVPMTAVP PFMDGLQPGY RQIQSPFPHT NSEQKMYPAV TV | |

Seq ID NO: 45 DNA sequence
Nucleic Acid Accession #: NM_002285
Coding sequence: 55-3738 (underlined sequences correspond to start and
stop codons)

| | |
|---|---|
| 1 11 21 31 41 51 | |
| \| \| \| \| \| \| | |
| GGCCGAGCCT CGGCGGCGGC GGTAGCGGCG GCGGCGACGC TGACACCTCC CACCATGGAC | 60 |
| AGCTTCGACT TAGCCCTGCT CCAGGAATGG GACCTCGAGT CACTGTGTGT CTATGAACCA | 120 |
| GATAGAAATG CATTACGGAG GAAAGAACGA GAAAGAAGAA ATCAAGAAAC TCAACAGGAT | 180 |
| GATGGCACGT TTAATTCTAG TTACTCTCTC TTCAGTGAGC CCTACAAGAC TAACAAGGGG | 240 |
| GATGAACTCT CCAACCGGAT CCAGAACACT TTAGGCAATT ATGATGAAAT GAAAGACTTT | 300 |
| TTAACTGATA GAACCAATCA GAGTCATCTC GTTGGAGTTC CCAAACCTGG GGTTCCTCAG | 360 |
| ACTCCTGTGA ACAAGATCGA TGAACATTTT GTTGCAGATT CAAGAGCCCA GAACCAGCCC | 420 |
| TCGTCTATCT GTAGCACTAC AACTTCCACA CCAGCAGCTG TCCCCGTGCA GCAGAGTAAA | 480 |
| AGAGGCACTA TGGGCTGGCA GAAGGCTGGG CACCCACCCT CTGACGGCCA ACAGAGAGCA | 540 |
| ACACAACAGG GCTCTCTCAG GACCTTGCTT GGAGATGGTG TTGGCAGACA GCAGCCTCGG | 600 |
| GCCAAACAAG TGTGCAATGT GGAGGTGGGC CTTCAGACCC AGGAGAGGCC ACCTGCCATG | 660 |
| GCGGCCAAGC ACAGCAGCAG CGGACACTGT GTTCAGAACT TTCCTCCATC CCTAGCTTCA | 720 |
| AAACCCAGCC TGGTCCAGCA GAAACCGACC GCGTATGTGA GGCCAATGGA CGGCCAAGAT | 780 |
| CAGGCCCCTG ATGAGTCTCC TAAGCTGAAG TCGTCTTCGG AAACCAGCGT GCACTGCACA | 840 |
| TCATACAGGG GAGTCCCTGC CAGCAAGCCG GAGCCTGCCA GAGCCAAGGC CAAGCTCTCC | 900 |
| AAGTTCAGCA TCCCCAAGCA GGGGGAGGAG AGTAGATCTG GAGAAACCAA CAGCTGTGTT | 960 |
| GAAGAAATAA TCCGGGAGAT GACCTGGCTT CCACCACTTT CTGCTATTCA AGCACCTGGC | 1020 |
| AAAGTGGAAC CAACCAAATT TCCATTTCCA AATAAGGACT CTCAGCTTGT ATCCTCTGGA | 1080 |
| CACAATAATC CAAAGAAAGG TGATGCAGAG CCAGAGAGTC CAGACAATGG CACATCGAAT | 1140 |
| ACATCAATGC TGGAAGATGA CCTTAAGCTA AGCAGTGATG AAGAGGAGAA TGAACAGCAG | 1200 |
| GCAGCTCAGA GAACGGCTCT CCGCGCTCTC TCTGACAGCG CCGTGGTCCA GCAGCCCAAC | 1260 |
| TGCAGAACCT CGGTGCCTTC CAGCAAGGGC AGCAGCAGCA GCAGCAGCAG CGGCACGAGC | 1320 |
| AGCTCCTCCA GCGACTCAGA GAGCAGCTCC GGATCTGACT CGGAGACCGA GAGCAGCTCC | 1380 |
| AGCGAGAGTG AGGGCAGCAA GCCCCCCCAC TTCTCCAGCC CCGAGGCTGA ACCGGCATCC | 1440 |
| TCTAACAAGT GGCAGCTGGA TAAATGGCTA AACAAAGTTA ATCCCCACAA GCCTCCTATT | 1500 |
| CTGATCCAAA ATGAAAGCCA CGGGTCAGAG AGCAATCAGT ACTACAACCC GGTGAAAGAG | 1560 |
| GACGTCCAGG ACTGTGGGAA AGTCCCCGAC GTTTGCCAGC CCAGCCTGAG AGAGAAGGAG | 1620 |
| ATCAAGAGCA CTTGCAAGGA GGAGCAAAGG CCAAGGACAG CCAACAAGGC CCCTGGGAGT | 1680 |
| AAAGGCGTGA AGCAGAAGTC CCCGCCCGCG GCCGTGGCCG TGGCGGTGAG CGCAGCCGCC | 1740 |
| CCGCCACCCG CAGTGCCCTG TGCGCCCGCG GAGAACGCGC CCGCGCCTGC CCGGAGGTCC | 1800 |
| GCGGGCAAGA AGCCCACCAG GCGCACCGAG AGGACCTCAG CCGGGGACGG CGCCAACTGC | 1860 |
| CACCGGCCCG AGGAGCCCGC GGCCGCGGAC GCGCTGGGGA CGAGCGTGGT GGTCCCCCCG | 1920 |
| GAGCCCACCA AAACCAGGCC CTGTGGCAAC AACAGAGCGA GCCACCGCAA GGAGCTGCGC | 1980 |
| TCCTCCGTGA CCTGCGAGAA GCGCCGCACG CGGGGGCTAA GCAGGATCGT CCCCAAATCC | 2040 |
| AAGGAGTTCA TTGAGACAGA GTCGTCATCT TCATCCTCCT CCTCGGACTC CGACCTGGAG | 2100 |
| TCCGAGCAGG AGGAGTACCC TCTGTCCAAA GCACAGACCG TGGCTGCCTC TGCCTCCTCC | 2160 |
| GGGAATGATC AGAGGCTGAA GGAGGCCGCT GCCAACGGGG GCAGTGGTCC TAGGGCCCCT | 2220 |
| GTAGGCTCCA TCAACGCCAG GACCACCAGT GACATCGCCA AGGAGCTGGA GGAGCAGITC | 2280 |
| TACACACTGG TCCCCTTTGG CCGGAACGAA CTTCTCTCCC CTCTAAAGGA CAGTGATGAG | 2340 |
| ATCAGGTCTC TCTGGGTCAA AATCGACCTG ACCCTCCTGT CCAGGATCCC AGAACACCTG | 2400 |
| CCCCAGGAGC CAGGGGTATT GAGCGCCCCT GCCACCAAGG ACTCTGAGAG CGCACCGCCC | 2460 |
| AGCCACACCT CGGACACACC TGCAGAAAAG GCTTTGCCAA AATCCAAGAG GAAACGCAAG | 2520 |
| TGTGACAACG AAGACGACTA CAGGGAGATC AAGAAGTCCC AGGGAGAGAA AGACAGCTCT | 2580 |
| TCAAGACTGG CCACCTCCAC CAGTAATACT TTGTCTGCAA ACCACTGCAA CATGAACATC | 2640 |
| AACAGTGTGG CAATACCAAT AAATAAAAAT GAAAAAATGC TTCGGTCGCC CATCTCACCC | 2700 |
| CTCTCTGATG CATCTAAACA CAAATACACC AGCGAGGACT TAACTTCTTC CAGCCGACCT | 2760 |
| AATGGCAACA GTTTGTTTAC TTCAGCCTCT TCCAGCAAAA AGCCTAAGGC CGACAGCCAG | 2820 |
| CTGCAGCCTC ACGGCGGAGA CCTCACGAAA GCAGCTCACA ACAATTCTGA AAACATTCCC | 2880 |
| CTCCACAAGT CACGGCCGCA GACGAAGCCG TGGTCTCCAG GCTCCAACGG CCACAGGGAC | 2940 |
| TGCAAGAGGC AGAAACTfGT CTTCGATGAT ATGCCTCGCA GTGCCGATTA TTTTATGCAA | 3000 |
| GAAGCTAAAC GAATGAAGCA TAAAGCAGAT GCAATGGTGG AAAAGTTTGG AAAGGCTTTG | 3060 |
| AACTATGCTG AAGCAGCATT GTCGTTTATC GAGTGTGGAA ATGCAATGGA ACAAGGCCCC | 3120 |
| ATGGAATCCA AATCTCCTTA TTACCTGATG TATTCAGAAA CAGTAGAGCT CATCAGGTAT | 3180 |
| GCTATGAGAC TAAAAACCCA CTCAGGCCCC AATGCCACAC CAGAAGACAA ACAACTGGCT | 3240 |
| GCATTATGTT ACCGATGCCT GGCCCTCCTG TACTGGCGGA TGTTTCGACT CAAAAGGGAC | 3300 |
| CACGCTGTAA AGTATTCAAA AGCACTAATC GACTATTTCA AGAACTCATC TAAAGCCGCC | 3360 |
| CAAGCCCCAT CTCCGTGGGG GGCCAGTGGA AAGAGCACTG GAACCCCATC CCCCATTTCT | 3420 |
| CCCAACCCCT TTCCCGGCAG CTCCGTGGGG TCTCAGGGCA GCCTCTCCAA CGCCAGCGCC | 3480 |
| CTGTCCCCGT CGACCATCGT CAGCATCCCA CAGCGCATCC ACCAGATGGC GGCCAACCAC | 3540 |
| GTCAGCATCA CCAACAGCAT CCTGCACAGC TACGACTACT GGGAGATGGC CGACAACCTG | 3600 |
| GCCAAGGAAA ACCGAGAATT CTTCAACGAC CTGGATCTGC TCATGGGGCC GGTCACCCTG | 3660 |
| CACAGCAGCA TGGAGCACCT GGTCCAGTAC TCCCAACAGG GCCTGCACTG GCTGCGGAAC | 3720 |
| AGCGCCCACC TGTCATAGGG ACCTCACCCT GGGGCCAGAG TGGGCTCTGG TCTCCACAGA | 3780 |
| TGGCTCAACG TTTTTGGACA CTGTGCTACT GAAACTCCCA GCCACAGCAT TTATAGACTG | 3840 |
| CGGTGAACAT TTCCTCA | |

Seq ID NO: 46 Protein sequence:
Protein Accession #: NP_002276

| | |
|---|---|
| 1 11 21 31 41 51 | |
| \| \| \| \| \| \| | |
| MDSFDLALLQ EWDLESLCVY EPDRNALRRK ERERRNQETQ QDDGTFNSSY SLFSEPYKTN | 60 |
| KGDELSNRIQ NTLGNYDEMK DFLTDRTNQS HLVGVPKPGV PQTPVNKIDE HVADSRAQN | 120 |
| QPSSICSTTT STPAAVPVQQ SKRGTMGWQK AGHPPSDGQQ RATQQGSLRT LLGDGVGRQQ | 180 |
| PRAXQVCNVE VGLQTQERPP AMAAKHSSSG HCVQNFPPSL ASKPSLVQQK PTAYVRPMDG | 240 |
| QDQAPDESPK LKSSSETSVH CTSYRGVPAS KPEPARAKAK LSKFSIPKQG EESRSGETNS | 300 |
| CVEEIIREMT WLPPLSAIQA PGKVEPTKFP FPNKDSQLVS SGHNNPKKGD AEPESPDNGT | 360 |
| SNTSMLEDDL KLSSDEEENE QQAAQRTALR ALSDSAVVQQ PNCRTSVPSS KGSSSSSSSG | 420 |
| TSSSSSDSES SSGSDSETES SSSESEGSKP PHFSSPEAEP ASSNKWQLDK WLNKVNPHKP | 480 |
| PILIQNBSHG SESNQYYNPV KBDVQDCGKV PDVCQPSLRE KEIKSTCKEE QRPRTANKAP | 540 |
| GSKGVKQKSP PAAVAVAVSA AAPPPAVPCA PAENAPAPAR RSAGKKPTRR TERTSAGDGA | 600 |
| NCHRPEEPAA ADALGTSVVV PPEPTKTRPC GNNRASHRKE LRSSVTCEKR RTRGLSRIVP | 660 |
| KSKEFIETES SSSSSSSDSD LESEQEEYPL SKAQTVAASA SSGNDQRLKE AAANGGSGPR | 720 |
| APVGS1NART TSDIAXELEE QFYThVPFGR NELLSPLKDS DEIRSLWVKI DLTLLSRIPE | 780 |
| HLPQEPGVLS APATKDSESA PPSHTSDTPA EKALPKSKRK RKCDNEDDYR EIKKSQGEKD | 840 |
| SSSRLATSTS NTLSANHCNM N1NSVAIPIN KNEKMLRSPI SPLSDASKHK YTSEDLTSSS | 900 |
| RPNGNSLFTS ASSSKKPKAD SQLQPHGGDL TKAAHNNSEN IPLHKSRPQT KPWSPGSNGH | 960 |
| RDCKRQKLVF DDMPRSADYF MQEAKRMKHK ADAMVEKFGK ALNYAEAALS FIECGNAMEQ | 1020 |
| GPMESKSPYY LMYSETVELI RYAMRLKTHS GPNATPEDKQ LAALCYRCLA LLYWRMFRLK | 1080 |
| RDHAVKYSKA LIDYFKNSSK AAQAPSPWGA SGKSTGTPSP ISPNPFPGSS VGSQGSLSNA | 1140 |
| SALSPSTIVS IPQRIHQMAA NHVSITNS1L HSYDYWEMAD NLAKENREFF NDLDLLMGPV | 1200 |
| TLHSSMEHLV QYSQQGLHWL RNSAHLS | |

Seq ID NO: 47 DNA sequence
Nucleic Acid Accession #: NM_033151
Coding sequence: 351-4499 (underlined sequences correspond to start
and stop codons)

| | |
|---|---|
| 1 11 21 31 41 51 | |
| \| \| \| \| \| \| | |
| ACTGGGATAA AGCAAGAAGA CTGATTTTAT GAGCAGGGGT TTGATACATC AAAGGAGATT | 60 |
| GCCCAGGATC AAGGGTGCGG TGTTGGGGGT GGGTTGGGGA GGGTGGTTAG AGAAGGTTTC | 120 |
| ACTAAGTGAT TTGGGCCTGA GGCCTGAGAA GATGTTTAAA AAGAGGGATC AAGCACAGGC | 180 |
| TAAGGAGAGG AAAGAGCAGG CACCCAAACC TCTGCATGGC CCCAATATGC TCCCTGCAGG | 240 |
| GTAGTGCCCC CTCTTCTGGC TGCTCAAGGC GAGATCTAAG CTTCTTCTAA CTCCTGCTGT | 300 |
| CTTTTCATAT TCTCTGATTC TGGGAAACGA AGAATTGGCA GGAACTGAAA ATGACTAGGA | 360 |
| AGAGGACATA CTGGGTGCCC AACTCTTCTG GTGGCCTCGT GAATCGTGGC ATCGACATAG | 420 |
| GCGATGACAT GGTTTCAGGA CTTATTTATA AAACCTATAC TCTCCAAGAT GGCCCCTGGA | 480 |
| GTCAGCAAGA GAGAAATCCT GAGGCTCCAG GGAGGGCAGC TGTCCCACCG TGGGGGAAGT | 540 |
| ATGATGCTGC CTTGAGAACC ATGATTCCCT TCCGTCCCAA GCCGAGGGTT CCTGCCCCCC | 600 |
| AGCCCCTGGA CAATGCTGGC CTGTTCTCCT ACCTCACCGT GTCATGGCTC ACCCCGCTCA | 660 |
| TGATCCAAAG CTTACGGAGT CGCTTAGATG AGAACACCAT CCCTCCACTG TCAGTCCATG | 720 |
| ATGCCTCAGA CAAAAATGTC CAAAGGCTTC ACCGCCTTTG GGAAGAAGAA GTCTCAAGGC | 780 |
| GAGGGATTGA AAAAGCTTCA GTGCTTCTGG TGATGCTGAG GTTCCAGAGA ACAAGGTTGA | 840 |
| TTTTCGATGC AGTTCTGGGC ATCTGCTTCT GCATTGCCAG TGTACTCGGG CCAATATTGA | 900 |
| TTATACCAAA GATCCTGGAA TATTCAGAAG AGCAGTTGGG GAATGTTGTC CATGGAGTGG | 960 |
| GACTCTGCTT TGCCCTTYGT CTCTCCGAAT GTGTGAAGTC TCTGAGTTTC TCCTCCAGTT | 1020 |
| GGATCATCAA CCAACGCACA GCCATCAGGT TCCGAGCAGC TGTTTCCTCC TTTGCCTTTG | 1080 |
| AGAAGCTCAT CCAATTTAAG TCTGTAATAC ACATCACCTC AGGAGAGGCC ATCAGCTTCT | 1140 |
| TCACCGGTGA TGTAAACTAC CTGTTTGAAG GGGTGTGCTA TGGACCCCTA GTACTGATCA | 1200 |
| CCTGCGCATC GCTGGTCATC TGCAGCATTT CTTCCTACTT CATTATTGGA TACACTGCAT | 1260 |
| TTATTGCCAT CTTATGCTAT CTCCTGGTTT TCCCACTGGC GGTATTCATG ACAAGAATGG | 1320 |
| CTGTGAAGGC TCAGCATCAC ACATCTGAGG TCAGCGACCA GCGCATCCGT GTGACCAGTG | 1380 |
| AAGTTCTCAC TTGCA1TAAG CTGA1TAAAA TGTACACATG GGAGAAACCA TTTGCAAAAA | 1440 |
| TCATTGAAGA CCTAAGAAGG AAGGAAAGGA AACTATTGGA GAAGTGCGGG CTTGTCCAGA | 1500 |
| GCCTGACAAG TATAACCTTG TTCATCATCC CCACAGTGGC CACAGCGGTC TGGGTTCTCA | 1560 |
| TCCACACATC CTTAAAGCTG AAACTCACAG CGTCAATGGC CTTCAGCATG CTGGCCTCCT | 1620 |
| TGAATCTCCT TCGGCTGTCA GTGTTCTTTG TGCCTATTGC AGTCAAAGGT CTCACGAATT | 1680 |
| CCAAGTCTGC AGTGATGAGG TTCAAGAAGT TTTTCCTCCA GGAGAGCCCT GTTTTCTATG | 1740 |
| TCCAGACATT ACAAGACCCC AGCAAAGCTC TGGTCTTTGA GGAGGCCACC TTGTCATGGC | 1800 |
| AACAGACCTG TCCCGGGATC GTCAATGGGG CACTGGAGCT GGAGAGGAAC GGGCATGCTT | 1860 |
| CTGAGGGGAT GACCAGGCCT AGAGATGCCC TCGGGCCAGA GGAAGAAGGG AACAGCCTGG | 1920 |
| GCCCAGAGTT GCACAAGATC AACCTGGTGG TGTCCAAGGG GATGATGTTA GGGGTCTGCG | 1980 |
| GCAACACGGG GAGTGGTAAG AGCAGCCTGT TGTCAGCCAT CCTGGAGGAG ATGCAC3TGC | 2040 |
| TCGAGGGCTC GGTGGGGGTG CAGGGAAGCC TGGCCTATGT CCCCCAGCAG GCCTGGATCG | 2100 |
| TCAGCGGGAA CATCAGGGAG AACATCCTCA TGGGAGGCGC ATATGACAAG GCCCGATACC | 2160 |
| TCCAGGTGCT CCACTGCTGC TCCCTGAATC GGGACCTGGA ACTTCTGCCC TTTGGAGACA | 2220 |
| TGACAGAGAT TGGAGAGCGG GGCCTCAACC TCTCTGGGGG GCAGAAACAG AGGATCAGCC | 2280 |
| TGGCCCGCGC CGTCTATTCC GACCGTCAGA TCTACCTGCT GGACGACCCC CTGTCTGCTG | 2340 |
| TGGACGCCCA CGTGGGGAAG CACATTTTTG AGGAGTGCAT TAAGAAGACA CTCAGGGGGA | 2400 |
| AGACGGTCGT CCTGGTGACC CACCAGCTGC AGTACTTAGA ATTTTGTGGC CAGATCATTT | 2460 |
| TGTTGGAAAA TGGGAAAATC TGTGAAAATG GAACTCACAG TGAGTTAATG CAGAAAAAGG | 2520 |
| GGAAATATGC CCAACTTATC CAGAAGATGC ACAAGGAAGC CACTTCGGAC ATGTTGCAGG | 2580 |
| ACACAGCAAA GATAGCAGAG AAGCCAAAGG TAGAAAGTCA GGCTCTGGCC ACCTCCCTGG | 2640 |
| AAGAGTCTCT CAACGGAAAT GCTGTGCCGG AGCATCAGCT CACACAGGAG GAGGAGATGG | 2700 |
| AAGAAGGCTC CTTGAGTTGG AGGGTCTACC ACCACTACAT CCAGGCAGCT GGAGGTTACA | 2760 |
| TGGTCTCTTG CATAATTFTC TTCTTCGTGG TGCTGATCGT CTTCTTAACG ATCTTCAGCT | 2820 |
| TCTGGTGGCT GAGCTACTGG TTGGAGCAGG GCTCGGGGAC CAATAGCAGC CGAGAGAGCA | 2880 |
| ATGGAACCAT GGCAGACCTG GGCAACATTG CAGACAATCC TCAACTGTCC TTCTACCAGC | 2940 |
| TGGTGTACGG GCTCAACGCC CTGCTCCTCA TCTGTGTGGG GGTCTGCTCC TCAGGGATTT | 3000 |
| TCACCAAAGT CACGAGGAAG GCATCCACGG CCCTGCACAA CAAGCTCTTC AACAAGGTTT | 3060 |
| TCCGCTGCCC CATGAGTTTC TTTGACACCA TCCCAATAGG CCGGCTTTTG AACTGCTTCG | 3120 |
| CAGGGGACTT GGAACAGCTG GACCAGCTCT TGCCCATCTT TTCAGAGCAG TTCCTGGTCC | 3180 |
| TGTCCTTAAT GGTGATCGCC GTCCTGTTGA TTGTCAGTGT GCTGTCTCCA TATATCCTGT | 3240 |
| TAATGGGAGC CATAATCATG GTTATTTGCT TCATTTATTA TATGATGTTC AAGAAGGCCA | 3300 |
| TCGGTGTGTT CAAGAGACTG GAGAACTATA GCCGGTCTCC TTTATTCTCC CACATCCTCA | 3360 |
| ATTCTCTGCA AGGCCTGAGC TCCATCCATG TCTATGGAAA AACTGAAGAC TTCATCAGCC | 3420 |
| AGTTTAAGAG GCTGACTGAT GCGCAGAATA ACTACCTGCT GTTGTTTCTA TCTTCCACAC | 3480 |
| GATGGATGGC ATTGAGGCTG GAGATCATGA CCAACCTTGT GACCTTGGCT GTTGCCCTGT | 3540 |
| TCGTGGCTTT TGGCATTTCC TCCACCCCCT ACTCCTTTAA AGTCATGGCT GTCAACATCG | 3600 |
| TGCTGCAGCT GGCGTCCAGC TTCCAGGCCA CTGCCCGGAT TGGCTTGGAG ACAGAGGCAC | 3660 |
| AGTTCACGGC TGTAGAGAGG ATACTGCAGT ACATGAAGAT GTGTGTCTCG GAAGCTCCTT | 3720 |
| TACACATGGA AGGCACAAGT TGTCCCCAGG GGTGGCCACA GCATTGGGAA ATCATATTTC | 3780 |
| AGGATTATCA CATGAAATAC AGAGACAACA CACCCACCGT GCTTCACGGC ATCAACCTGA | 3840 |
| CCATCCGCGG CCACGAAGTG GTGGGCATCG TGGGAAGGAC GGGCTCTGGG AAGTCCTCCT | 3900 |
| TGGGCATGGC TCTCTTCCGC CTGGTGGAGC CCATGGCAGG CCGGATTGTC ATTGACGGCG | 3960 |
| TGGACATTTG CAGCATCGGC CTGGAGGACT TGCGGTCCAA GCTCTCAGTG ATCCCTCAAG | 4020 |
| ATCCAGTGCT GCTCTCAGGA ACCATCAGAT TCAACCTAGA TCCCTTTGAC CGTCACACTG | 4080 |
| ACCAGCAGAT CTGGGATGCC TTGGAGAGGA CATTCCTGAC CAAGGCCATC TCAAAGTTCC | 4140 |
| CCAAAAAGCT GCATACAGAT GTGGTGGAAA ACGGTGGAAA CTTCTCTGTG GGGGAGAGGC | 4200 |
| AGCTGCTCTG CATTGCCAGG GCTGTGCTTC GCAACTCCAA GATCATCCTT ATCGATGAAG | 4260 |
| CCACAGCCTC CATTGACATG GAGACAGACA CCCTGATCCA GCGCACAATC CGTGAAGCCT | 4320 |
| TCCAGGGCTG CACCGTGCTC GTCAITGCCC ACCGTGTCAC CACTGTGCTG AACTGTGACC | 4380 |
| ACATCCTGGT TATGGGCAAT GGGAAGGTGG TAGAATTTGA TCGGCCGGAG GTACTGCGGA | 4440 |
| AGAAGCCTGG GTCATTGTTC GCAGCCCTCA TGGCCACAGT CACTTCTTCA CTGAGATAAG | 4500 |
| GAGATGTGGA GACYECATGG AGGCTGGCAG CTGAGCTCAG AGGTTCACAC AGGTGCAGCT | 4560 |
| TCGAGGCCCA CAGTCTGCGA CCTTCTTGTT TGGAGATGAG AACTTCTCCT GGAAGCAGGG | 4620 |
| GTAAATGTAG GGGGGGTGGG GATTGCTGGA TGGAAACCCT GGAATAGGCT ACTTGATGGC | 4680 |
| TCTCAAGACC TTAGAACCCC AGAACCATCT AAGACATGGG ATTCAGTGAT CATGTGGTTC | 4740 |
| TCCTTTTAAC TTACATGCTG AATAATTTTA TAATAAGGTA AAAGCTTATA GTTTTCTGAT | 4800 |
| CTGTGTTAGA AGTGTTGCAA ATGCTGTACT GACTTTGTAA AATATAAAAC TAAGGAAAAC | 4860 |
| TC | |

Seq ID NO: 48 Protein sequence:
Protein Accession #: NP_149163.2
MTRKRTYWVPNSSGGLVNRGIDIGDDMVSGLIYKYYTLQDGPWSQQERNPEAPGRAAVPPWGKYDAALRT
MIPFRPKPRFPAPQPLDNAGLFSYLTVSWLTPLMIQSLRSRLDENTIPPLSVHDASDKNVQRLHRLWEEE
VSRRGIEKASVLLVMLRFQRTRLIFDALLGICFCIASVLGPILIIPKILEYSEEQLGNVVNGVGLCFALF
LSECVKSLSFSSSWIINQRTAIRFRAAVSSFAFEKLIQFKSVIHITSGEAISFFTGDVNYLFEGVCYGPL
VLITCASLVICSISSYF1IGYTAEIAILCYLLVFPLAVFMTRMAVKAQHHTSEVSDQRIRVTSEVLTCIK
LIKMYTWEKPFAXIIEDLRRKERKLLEKCGLVQSLTSITLFIIPTVATAVWVLIHTSLKLKLTASMAFSM
LASLNLLRLSVFFVPIAVKGLTNSKSAVMRFKKFFLQESPVFYVQTLQDPSKALVFEEATLSWQQTCPGI
VNGALELERNGHASEGMTRPRDALGPEEEGNSLGPELHK1NLVVSKGMMLGVCGNTGSGKSSLLSMLEEM
HLLEGSVGVQGSLAYVPQQAWIVSGNIRENILMGGAYD1CARYLQVLHCCSLNRDLELLPFGDMTEIGER
GLNLSGGQKQRSLARAVYSDRQIYLLDDPLSAVDAHVGKHIFEECIKKTLRGKTVVLVTHQLQYLEFCG
QIILLENGKICENGTHSELMQKKGKYAQLIQKMHKEATSDMLQDTAKIAEKPKVESQALATSLEESLNGN
AVPEHQLTQEEEMEEGSLSWRVYHHYIQAAGGYMVSCIIFFFVVLIVFLTIFSEWWLSYWLEQGSGTNSS
RESNGTMADLGNIADNPQLSFYQLVYGLNALLLICVGVCSSGIFTKVTRKASTALHNKLFNKVFRCPMSF
FDTIPIGRLLNCFAGDLEQLDQLLPIFSEQFLVLSLMVIAVLLIVSVLSPYILLMGAIIMVICFIYYMMF
KKAIGVFKRLENYSRSPLFSHILNSLQGLSSIHVYGKTEDFISQEKRLTDAQNNYLLLFLSSTRWMALRL
EIMTNLVTLAVALFVAFGISSTPYSFKVMAVNtVLQLASSFQATARIGLETEAQFTAVERILQYMKMCVS
EAPLHMEGTSCPQGWPQHGEIIFQDYHMKYRDNTPTVLHGINLTIRGHEVVGIVGRTGSGKSSLGMALFR
LVEPMAGRILIDGVDICSIGLEDLRSKLSVIPQDPVLLSGTIRFNLDPFDRHTDQQIWDALERTFLTKAI
SKFPKXLHTDVVENGGNFSVGERQLLCIARAVLRNSKIILIDEATASIDMETDTLIQRTJREAFQGCTVL
VIAHRVTTVLNCDHILVMGNGKVVEEDRPEVLRXICPGSLFAALMATATSSLR
Seq ID NO: 49 DNA sequence
Nucleic Acid Accession #: NM_033419
Coding sequence: 18-980 (underlined sequences correspond to start and
stop codons)

| | |
|---|---|
| 1 11 21 31 41 51 | |
| \| \| \| \| \| \| | |
| CGAGCCAGGG AGAAAGGATG GCCGGCCTGG CGGCGCGGTT GGTCCTGCTA GCTGGGGCAG | 60 |
| CGGCGCTGGC GAGCGGCTCC CAGGGCGACC GTGAGCCGGT GTACCGCGAC TGCGTACTGC | 120 |
| AGTGCGAAGA GCAGAACTGC TCTGGGGGCG CTCTGAATCA CTTCCGCTCC CGCCAGCCAA | 180 |
| TCTACATGAG TCTAGCAGGC TGGACCTGTC GGGACGACTG TAAGTATGAG TGTATGTGGG | 240 |
| TCACCGTTGG GCTCTACCTC CAGGAAGGTC ACAAAGTGCC TCAGTTCCAT GGCAAGTGGC | 300 |
| CCTTCTCCCG GTTCCTGTTC TTTCAAGAGC CGGCATCGGC CGTGGCCTCG TTTCTCAATG | 360 |
| GCCTGGCCAG CGTGGTGATG CTCTGCCGCT ACCGCACCTT CGTGCCAGCC TCCTCCCCCA | 420 |
| TGTACCACAC CTGTGTGGCC TTCGCCTGGG TGTCCCTCAA TGCATGGTTC TGGTCCACAG | 480 |
| TTTTCCACAC CAGGGACACT GACCTCACAG AGAAAATGGA CTACTTCTGT GCCTCCACTG | 540 |
| TCATCCTACA CTCAATCTAC CTGTGCTGCG TCAGGACCGT GGGGCTGCAG CACCCAGCTG | 600 |
| TGGTCAGTGC CTTCCGGGCT CTCCTGCTGC TCATGCTGAC CGTGCACGTC TCCTACCTGA | 660 |
| GCCTCATCCG CTTCGACTAT GGCTACAACC TGGTGGCCAA CGTGGCTATT GGCCTGGTCA | 720 |
| ACGTGGTGTG GTGGCTGGCC TGGTGCCTGT GGAACCAGCG GCGGCTGCCT CACGTGCGCA | 780 |
| AGTGCGTGGT GGTGGTCTTG CTGCTGCAGG GGCTGTCCTT GCTCGAGCTG CTTGACTTCC | 840 |
| CACCGCTCTT CTGGGTCCTG GATGCCCATG CCATCTGGCA CATCAGCACC ATCCCTGTCC | 900 |
| ACGTCCTC1T TTTCAGCTTT CTGGAAGATG ACAGCCTGTA CCTGCTGAAG GAATCAGAGG | 960 |
| ACAAGTTCAA GCTGGACTGA AGACCTTGGA GCGAGTCTGC CCCAGTGGGG ATCCTGCCCC | 1020 |
| CGCCCTGCTG GCCTCCCTTC TCCCCTCAAC CCTTGAGATG ATTTTCTCTT TTCAACTTCT | 1080 |
| TGAACTTGGA CATGAAGGAT GTGGGCCCAG AATCATGTGG CCAGCCCACC CCCTGTTGGC | 1140 |
| CCTCACCAGC CTTGGAGTCT GTTCTAGGGA AGGCCTCCCA GCATCTGGGA CTCGAGAGTG | 1200 |
| GGCAGCCCCT CTACCTCCTG GAGCTGAACT GGGGTGGAAC TGAGTGTGCT CTTAGCTCTA | 1260 |
| CCGGGAGGAC AGCTGCCTGT 1TCCTCCCCA TCAGCCTCCT CCCCACATCC CCAGCTGCCT | 1320 |
| GGCTGGGTCC TGAAGCCCTC TGTCTACCTG GGAGACCAGG GACCACAGGC CTTAGGGATA | 1380 |
| CAGGGGGTCC CCTTCTGTTA CCACCCCCCA CCCTCCTCCA GGACACCACT AGGTGGTGCT | 1440 |
| GGATGCTTGT TCTTTGGCCA GCCAAGGTTC ACGGCGATTC TCCCCATGGG ATCTTGAGGG | 1500 |
| ACCAAGCTGC TGGGATTGGG AAGGAGTTTC ACCCTGACCA TTGCCCTAGC CAGGTTCCCA | 1560 |
| GGAGGCCTCA CCATACTCCC TTTCAGGGCC AGGGCTCCAG CAAGCCCAGG GCAAGGATCC | 1620 |
| TGTGCTGCTG TCTGGTTGAG AGCCTGCCAC CGTGTGTCGG GAGTGTGGGC CAGGCTGAGT | 1680 |
| GCATAGGTGA CAGGGCCGTG AGCATGGGCC TGGGTGTGTG TGAGCTCAGG CCTAGGTGCG | 1740 |
| CAGTGTGGAG ACGGGTGTTG TCGGGGAAGA GGTGTGGCTT CAAAGTGTGT GTGTGCAGGG | 1800 |
| GGTGGGTGTG TTAGCGTGGG TTAGGGGAAC GTGTGTGCGC GTGCTGGTGG GCATGTGAGA | 1860 |
| TGAGTGACTG CCGGTGAATG TGTCCACAGT TGAGAGGTTG GAGCAGGATG AGGGAATCCT | 1920 |
| GTCACCATCA ATAATCACTT GTGGAGCGCC AGCTCTGCCC AAGGCGCCAC CTGGGCGGAC | 1980 |
| AGCCAGGAGC TCTCCATGGC CAGGCTGCCT GTGTGCATGT TCCCTGTCTG GTGCCCCTTT | 2040 |
| GCCCGCCTCC TGCAAACCTC ACAGGGTCCC CACACAACAG TGCCCTCCAG AAGCAGCCCC | 2100 |
| TCGGAGGCAG AGGAAGGAAA ATGGGGATGG CTGGGGCTCT CTCCATCCTC CTTTTCTCCT | 2160 |
| TGCCTTCGCA TGGCTGGCCT TCCCCTCCAA AACCTCCATT CCCCTGCTGC CAGCCCCTTT | 2220 |
| GCCATAGCCT GATTITGGGG AGGAGGAAGG GGCGATTTGA GGGAGAAGGG GAGAAAGCTT | 2280 |
| ATGGCTGGGT CTGGTTTCTT CCCTTCCCAG AGGGTCTTAC TGTTCCAGGG TGGCCCCAGG | 2340 |
| GCAGGCAGGG GCCACACTAT GCCTGCGCCC TGGTAAAGGT GACCCCTGCC ATITACCAGC | 2400 |
| AGCCCTGGCA TGTTCCTGCC CCACAGGAAT AGAATGGAGG GAGCTCCAGA AACTTTCCAT | 2460 |
| CCCAAAGGCA GTCTCCGTGG TTGAAGCAGA CTGGATTTTT GCTCTGCCCC TGACCCCTTG | 2520 |
| TCCCTGTTTG AGGGAGGGGA GCTATGCTAG GACTCCAACC TCAGGGACTC GGGTGGCCTG | 2580 |
| CGCTAGCTTC TTTTGATACT GAAAACTTTT AAGGTGGGAG GGTGGCAAGG GATGTGCTTA | 2640 |
| ATAAATCAAT TCCAAGCCTC AAAAAAAAAA AAAAAAAAAA AA | |

Seq ID NO: 50 Protein sequence:
Protein Accession #: NP_219487.1

| | |
|---|---|
| 1 11 21 31 41 51 | |
| \| \| \| \| \| \| | |
| MKDVGPESCG QPTPCWPSPA LESVLGKASQ HLGLESGQPL YLLELNWGGT ECALSSTGRT | 60 |
| AACFLPISLL PTSPAAWLGP EALCLPGRPG TTGLRDTGGP LLLPPPTLLQ DTTRWCWMLV | 120 |
| LWPAKVHGDS PHGILRDQAA GIGKEFHPDH CPSQVPRRPH HTPFQGQGSS KPRARILCCC | 180 |
| LVESLPPCVG SVGQAECIGD RAVSMGLGVC ELRPRCAVWR RVLSGKRCGF KVCVCRGWVC | |

Seq ID NO: 51 DNA sequence
Nucleic Acid Accession #: XM_059098.1
Coding sequence: 178-618 (underlined sequences correspond to start and
stop codons)

| | |
|---|---|
| 1 11 21 31 41 51 | |
| \| \| \| \| \| \| | |
| GATGTACACT CTGAAGTGAG CACATTCCTG TTGGCAGGAC ATGACACCTT GGCAGCAAGC | 60 |
| ATCTCCTGGA TCCYfTACTG CCTGGCTCTG AACCCTGAGC ATCAAGAGAG ATGCCGGGAG | 120 |
| GAGGTCAGGG GCATCCTGGG GGATGGGTCT TCTATCACTT GGGACCAGCT GGGTGAGATG | 180 |
| TCGTACACCA CAATGTGCAT CAAGGAGACG TGCCGATTGA TTCCTGCAGT CCCGTCCATT | 240 |
| TCCAGAGATC TCAGCAAGCC ACTTACCTTC CCAGATGGAT GCACATTGCC TGCAGGGATC | 300 |
| ACCGTGGTTC TTAGTATTTG GGGTCTTCAC CACAACCCTG CTGTCTGGAA AAACCCAAAG | 360 |
| GTCTTTGACC CCTTGAGGTT CTCTCAGGAG AATTCTGATC AGAGACACCC CTATGCCTAC | 420 |
| TTACCATTCT CAGCTGGATC AAGGAACTGC ATTGGGCAGG AGTTTGCCAT GATTGAGTTA | 480 |
| AAGGTAACCA TTGCCTTGAT TCTGCTCCAC TTCAGAGTGA CTCCAGACCC CACCAGGCCT | 540 |
| CTTACTTTCC CCAACCATTT TATCCTCAAG CCCAAGAATG GGATGTATTT GCACCTGAAG | 600 |
| AAACTCTCTG AATGTTAGAT CTCAGGGTAC AATGATTAAA CGTACTTTGT TTTTCGAAGT | 660 |
| TAAATTTACA GCTAATGATC CAAGCAGATA GAAAGGGATC AATGTATGGT GGGAGGATTG | 720 |
| GAGGGTGGTG GGATAGGGGT CTCTGTGAAG AGATCCAAAA TCATTTCTAG GTACACAGTG | 780 |
| TGTCAGCTAG ATCTGTTTCT ATATAACGTT GGGAGATTTT CAGATCTTTT CTGTTAAACT | 840 |
| TTCACTACTA TTAATGCTGT ATACACCAAT AGACGTTCAT ATATTTTCTG TTGTTTTTAA | 900 |
| AATAGTTTTC AGAATTATGC AAGTAATAAG TGCATGTATG CTCACTGTCA AAAATTCCCA | 960 |
| ACACTAGAAA ATCATGTAGA ATAAAAATTT TAAATCTCAC TTCACTTAGC CGACATTCCA | 1020 |
| TGCCCTGACC AATCCTACTG CTTTTCCTAA AAACAGAATA ATTTGGTGTG CATGTTTTCA | 1080 |
| GACTTTTTCC TATACATTTT ATATGTAGAA ATGTAGCAAT GTATTTGTAT AGATGTGATC | 1140 |
| ATTCCTATAT TGTTATTGAT TTTTTTCACT TAATAAAAAT TCACCTTATT CCTT | |

Seq ID NO: 52 Protein sequence:
Protein Accession #: XP_059098.1

| | |
|---|---|
| 1 11 21 31 41 51 | |
| \| \| \| \| \| \| | |
| MSYTTMCIKE TCRLIPAVPS ISRDLSKPLT FPDGCTLPAG ITVVLSIWGL HHNPAVWKNP | 60 |
| KVFDPLRFSQ ENSDQRHPYA YLPFSAGSRN CIGQEFAMIE LKVTIALILL HFRVTPDPTR | 120 |
| PLTEPNHEIL KPKNGMYLHL KKLSEC | |

Seq ID NO: 53 DNA sequence
Nucleic Acid Accession #: NM_030916
Coding sequence: 1-1533 (underlined sequences correspond to start and
stop codons)

| | |
|---|---|
| 1 11 21 31 41 51 | |
| \| \| \| \| \| \| | |
| ATGCCCCTGT CCCTGGGAGC CGAGATGTGG GGGCCTGAGG CCTGGCTGCT GCTGCTGCTA | 60 |
| CTGCTGGCAT CATTTACAGG CCGGTGCCCC GCGGGTGAGC TGGAGACCTC AGACGTGGTA | 120 |
| ACTGTGGTGC TGGGCCAGGA CGCAAAACTG CCCTGCTTCT ACCGAGGGGA CTCCGGCGAG | 180 |
| CAAGTGGGGC AAGTGGCATG GGCTCGGGTG GACGCGGGCG AAGGCGCCCA GGAACTAGCG | 240 |
| CTACTGCACT CCAAATACGG GCTTCATGTG AGCCCGGCTT ACGAGGGCCG CGTGGAGCAG | 300 |
| CCGCCGCCCC CACGCAACCC CCTGGACGGC TCAGTGCTCC TGCGCAACGC AGTGCAGGCG | 360 |
| GATGAGGGCG AGTACGAGTG CCGGGTCAGC ACCTTCCCCG CCGGCAGCTT CCAGGCGCGG | 420 |
| CTGCGGCTCC GAGTGCTGGT GCCTCCCCTG CCCTCACTGA ATCCTGGTCC AGCACTAGAA | 480 |
| GAGGGCCAGG GCCTGACCCT GGCAGCCTCC TGCACAGCTG AGGGCAGCCC AGCCCCCAGC | 540 |
| GTGACCTGGG ACACGGAGGT CAAAGGCACA ACGTCCAGCC GTTCCTTCAA GCACTCCCGC | 600 |
| TCTGCTGCCG TCACCTCAGA GTTCCACTTT GTGCCTAGCC GCAGCATGAA TGGGCAGCCA | 660 |
| CTGACTTGTG TGGTGTCCCA TCCTGGCCTG CTCCAGGACC AAAGGATCAC CCACATCCTC | 720 |
| CACGTGTCCT TCCTTGCTGA GGCCTCTGTG AGGGGCCTTG AAGACCAAAA TCTGTGGCAC | 780 |
| ATTGGCAGAG AAGGAGCTAT GCTCAAGTGC CTGAGTGAAG GGCAGCCCCC TCCCTCATAC | 840 |
| AACTGGACAC GGCTGGATGG GCCTCTGCCC AGTGGGGTAC GAGTGGATGG GGACACTTTG | 900 |
| GGCTTTCCCC CACTGACCAC TGAGCACAGC GGCATCTACG TCTGCCATGT CAGCAATGAG | 960 |
| TTCTCCTCAA GGGATTCTCA GGTCACTGTG GATGTTCTTG ACCCCCAGGA AGACTCTGGG | 1020 |
| AAGCAGGTGG ACCTAGTGTC AGCCTCGGTG GTGGTGGTGG GTGTGATCGC CGCACTCTTG | 1080 |
| TTCTGCCTTC TGGTGGTGGT GGTGGTGCTC ATGTCCCGAT ACCATCGGCG CAAGGCCCAG | 1140 |
| CAGATGACCC AGAAATATGA GGAGGAGCTG ACCCTGACCA GGGAGAACTC CATCCGGAGG | 1200 |
| CTGCATTCCC ATCACACGGA CCCCAGGAGC CAGCCGGAGG AGAGTGTAGG GCTGAGAGCC | 1260 |
| GAGGGCCACC CTGATAGTCT CAAGGACAAC AGTAGCTGCT CTGTGATGAG TGAAGAGCCC | 1320 |
| GAGGGCCGCA GTTACTCCAC GCTGACCACG GTGAGGGAGA TAGAAACACA GACTGAACTG | 1380 |
| CTGTCTCCAG GCTCTGGGCG GGCCGAGGAG GAGGAAGATC AGGATGAAGG CATCAAACAG | 1440 |
| GCCATGAACC ATTTTGTTCA GGAGAATGGG ACCCTACGGG CCAAGCCCAC GGGCAATGGC | 1500 |
| ATCTACATCA ATGGGCGGGG ACACCTGGTC TGA | |

Seq ID NO: 54 Protein sequence:
Protein Accession #: NP_112178.1

| | |
|---|---|
| 1 11 21 31 41 51 | |
| \| \| \| \| \| \| | |
| MPLSLGAEMW GPEAWLLLLL LLASFTGRCP AGELETSDVV TVVLGQDAKL PCFYRGDSGE | 60 |
| QVGQVAWARV DAGEGAQELA LLHSKYGLHV SPAYEGRVEQ PPPPRNPLDG SVLLRNAVQA | 120 |
| DEGEYECRVS TFPAGSFQAR LRLRVLVPPL PSLNPGPALE EGQGLTLAAS CTAEGSPAPS | 180 |
| VTWDTEVKGT TSSRSFKHSR SAAVTSEFHL VPSRSMNGQP LTCVVSHPGL LQDQRITHIL | 240 |
| HVSFLAEASV RGLEDQNLWH IGREGAMLKC LSEGQPPPSY NWTRLDGPLP SGVRVDGDTL | 300 |
| GFPPLTTEHS GIYVCHVSNE FSSRDSQVTV DVLDPQEDSG KQVDLVSASV VVVGVIAALL | 360 |
| FCLLVVVVVL MSRYHRRKAQ QMTQKYEEEL TLTRENSIRR LHSHHTDPRS QPEESVGLRA | 420 |
| EGHPDSLKDN SSCSVMSEEP EGRSYSTLTT VRBIETQTEL LSPGSGRAEE EEDQDEGIKQ | 480 |
| AMNHFVQENG TLRAKPTGNG IYINGRGHLV | |

Seq ID NO: 55 DNA sequence
Nucleic Acid Accession #: AF007170.1
Coding sequence: 73-1725 (underlined sequences correspond to start and
stop codons)

| | |
|---|---|
| 1 11 21 31 41 51 | |
| \| \| \| \| \| \| | |
| AAGGAGGCGG CCTCCGGGAA AAGCGACCGC AGGACTCCTG AGAGCAGCCT CCATGAGGCC | 60 |
| CTGGACCAGT GCATGACCGC CCTGGACCTC TTCCTCACCA ACCAGTTCTC AGAAGCACTC | 120 |
| AGCTACCTCA AGCCCAGAAC CAAGGAAAGC ATGTACCACT CACTGACATA TGCCACCATC | 180 |
| CTGGAGATGC AGGCCATGAT GACCTTTGAC CCTCAGGACA TCCTGCTTGC CGGCAACATG | 240 |
| ATGAAGGAGG CACAGATGCT GTGTCAGAGG CACCGGAGGA AGTCTTCTGT AACAGATTCC | 300 |
| TTCAGCAGCC TGGTGAACCG CCCCACGCTG GGCCAATTCA CTGAAGAAGA AATCCACGCT | 360 |
| GAGGTCTGCT ATGCAGAGTG CCTGCTGCAG CGAGCAGCCC TGACCTTCCT GCAGGACGAG | 420 |
| AACATGGTGA GCTTCATCAA AGGCGGCATC AAAGTTCGAA ACAGCTACCA GACCTACAAG | 480 |
| GAGCTGGACA GCCTTGTTCA GTCCTCACAA TACTGCAAGG GTGAGAACCA CCCGCACTTT | 540 |
| GAAGGAGGAG TGAAGCTTGG TGTAGGGGCC TTCAACCTGA CACTGTCCAT GCTTCCTACT | 600 |
| AGGATCCTGA GGCTGTTGGA GTTTGTGGGG TTTTCAGGAA ACAAGGACTA TGGGCTGCTG | 660 |
| CAGCTGGAGG AGGGAGCGTC AGGGCACAGC TTCCGCTCTG TGCTCTGTGT CATGCTCCTG | 720 |
| CTGTGCTACC ACACCTTCCT CACCTTCGTG CTCGGTACTG GGAACGTCAA CATCGAGGAG | 780 |
| GCCGAGAAGC TCTTGAAGCC CTACCTGAAC CGGTACCCTA AGGGTGCCAT CTTCCTGTTC | 840 |
| TTTGCAGGGA GGATTGAAGT CATTAAAGGC AACATTGATG CAGCCATCCG GCGTTTCGAG | 900 |
| GAGTGCTGTG AGGCCCAGCA GCACTGGAAG CAGTTTCACC ACATGTGCTA CTGGGAGCTG | 960 |
| ATGTGGTGCT TCACCTACAA GGGCCAGTGG AAGATGTCCT ACTTCTACGC CGACCTGCTC | 1020 |
| AGCAAGGAGA ACTGCTGGTC CAAGGCCACC TACATTTACA TGAAGGCCGC CTACCTCAGC | 1080 |
| ATGTTTGGGA AGGAGGACCA CAAGCCGTTC GGGGACGACG AAGTGGAATT ATTTCGAGCT | 1140 |
| GTGCCAGGCC TGAAGCTCAA GATTGCTGGG AAATCTCTAC CCACAGAGAA GTTTGCCATC | 1200 |
| CGGAAGTCCC GGCGCTACTT CTCCTCCAAC CCTATCTCGC TGCCAGTGCC TGCTCTGGAA | 1260 |
| ATGATGTACA TCTGGAACGG CTACGCCGTG ATTGGGAAGC AGCCGAAACT CACGGATGGG | 1320 |
| ATACTTGAGA TTATCACTAA GGCTGAAGAG ATGCTGGAGA AAGGCCCAGA GAACGAGTAC | 1380 |
| TCAGTGGATG ACGAGTGCTT GGTGAAATTG TTGAAAGGCC TGTGTCTGAA ATACCTGGGC | 1440 |
| CGTGTCCAGG AGGCCGAGGA GAATTTTAGG AGCATCTCTG CCAATGAAAA GAAGATTAAA | 1500 |
| TATGACCACT ACTTGATCCC AAACGCCCTG CTOGAGCTGG CCCTGCTGCT TATGGAGCAA | 1560 |
| GACAGAAACG AAGAGGCCAT CAAACTTTTG GAATCTGCCA AGCAAAACTA CAAGAATTAC | 1620 |
| TCCATGGAGT CAAGGACACA CTTTCGAATC CAGGCAGCCA CACTCCAAGC CAAGTCTTCC | 1680 |
| CTAGAGAACA GCAGCAGATC CATGGTCTCA TCAGTGTCCT TGTAGCTTTG TGCAGCAGTT | 1740 |
| CCGGGCTGGA AGACAGAGAC AGCTGGACAG AGCTCCTGAA AACATTTCAA AATACCCCCT | 1800 |
| CCCCCTGCCC TGCCCTGCCT TTGGGGTCCA CCGGCACTCC AGTTGGATGG CACAACATAG | 1860 |
| TGTATCCGTG CAGAAGCCGA GCTGGCATTT TCACCAGTGT AGCCAAGGGC CTTTGCCAAG | 1920 |
| GGCAGAGCAG GTGGAGCCCT CTGCCTGCCC TATCACACAT ACGGGTACTT GCTTTTCACT | 1980 |
| GTGATGTTTA AGAGAATGTA TGAACAGTTT ACCTTTTCCT TAGAAATACA TTGATGGGAT | 2040 |
| CACAGTTGGC TTTAAAAACC AACAACAATC AACCACCTGT AAGTCTTTGT CTTCACCTAT | 2100 |
| TATCATCTGG AGGTAAATCT CTTTATATGA TGATGCCAAA GGGCAAATTG CTTTCAAAT | 2160 |
| TCAGCAAGTT CTCAGCTTGT GTGACGGAAG GTCCTTCAGA GGACCTGAGG AATGCCTGGG | 2220 |
| AGAGGCTAAG CCTCAGGCTT CAATGCTTCT GGGGTTGGGC ATGAGGATGT ACACAGACAC | 2280 |
| CCACTACCTT ACTACTCACA CTfCATTTCA CTCCTTTTGT AAATTTCCAA TTTAAAAATC | 2340 |
| AAGCACGTCT TTTTAGTGAG ATAAAATCTG AGCTCTTCTG TAGAAAAATC AATCTCTACC | 2400 |
| AGTAGAAAAT GCCAGGGCTT GATGGAAGAG CTGTGTAGCC CTTTCTATGC CAAAGCCAGG | 2460 |
| AAATTTGGGG GGCAGGAGGA GGTTCTCAGA ATCCAGTCTG TATCTTTGCT GTATGCCAAA | 2520 |
| CTGAAACCAC TGGGAATAAT TTATGAAACA TAAAAATCTT CTGTACTTCA CTCCAAGGTA | 2580 |
| CATTTGCTTA CTGACAGCAT TTTTGTTAAA ACTGTTATTC TTGAAAAAAA AAAAAAAAAA | 2640 |
| AA | |

Seq ID NO: 56 Protein sequence:
Protein Accession #: AAC39582.1

| | |
|---|---|
| 1 11 21 31 41 51 | |
| \| \| \| \| \| \| | |
| MTALDLFLTN QFSEALSYLK PRTKBSMYHS LTYATILEMQ AMMTFDPQDI LLAGNMMKEA | 60 |
| QMLCQRHRRK SSVTDSFSSL VNRPTLGQFT EEEIHAEVCY AECLLQRAAL TFLQDENVIVS | 120 |
| FIKGGIKVRN SYQTYKELDS LVQSSQYCKG ENHPHFEGGV KLGVGAFNLT LSMLPTRILR | 180 |
| LLEFVGFSGN KDYGLLQLEE GASGHSFRSV LCVMLLLCYH TFLTFVLGTG NVNIEEAEKL | 240 |
| LKPYLNRYPK GAIFLFFAGR IEVIKGNIDA AIRRFEECCE AQQHWKQFHH MCYWELMWCF | 300 |
| TYKGQWKMSY EYADLLSKEN CWSKATYIYM KAAYLSMFGK EDHXPFGDDE VELFRAVPGL | 360 |
| KLKIAGKSLP TEKFAIRKSR RYFSSNPISL PVPALEMMYI WNGYAVIGKQ PKLTDGILEI | 420 |
| ITKAEEMLEK GPENEYSVDD ECLVKLLKGL CLKYLGRVQE AEENFRSISA NEKKIKYDHY | 480 |
| LIPNALLELA LLLMEQDRNE EAIXLLESAK QNYKNYSMES RTHFRIQAAT LQAKSSLENS | 540 |
| SRSMVSSVSL | |

Seq ID NO: 57 DNA sequence
Nucleic Acid Accession #: NM_006670.1
Coding sequence: 1-927 (underlined sequences correspond to start and
stop codons)

| | |
|---|---|
| 1 11 21 31 41 51 | |
| \| \| \| \| \| \| | |
| ATGCCTGGGG GGTGCTCCCG GGGCCCCGCC GCCGGGGACG GGCGTCTGCG GCTGGCGCGA | 60 |
| CTAGCGCTGG TACTCCTGGG CTGGGTCTCC TCGTCTTCTC CCACCTCCTC GGCATCCTCC | 120 |
| TTCTCCTCCT CGGCGCCGTT CCTGGCTTCC GCCGTGTCCG CCCAGCCCCC GCTGCCGGAC | 180 |
| CAGTGCCCCG CGCTGTGCGA GTGCTCCGAG GCAGCGCGCA CAGTCAAGTG CGTTAACCGC | 240 |
| AATCTGACCG AGGTGCCCAC GGACCTGCCC GCCTACGTGC GCAACCTCTT CCTTACCGGC | 300 |
| AACCAGCTGG CCAGCAACCA GTTCCTTTAC CTGCCGCGGG ATGTGCTGGC CCAACTGCCC | 360 |
| AGCCTCAGGC ACCTGGACTT AAGTAATAAT TCGCTGGTGA GCCTGACCTA CGTGTCCTTC | 420 |
| CGCAACCTGA CACATCTAGA AAGCCTCCAC CTGGAGGACA ATGCCCTCAA GGTCCTTCAC | 480 |
| AATGGCACCC TGGCTGAGTT GCAAGGTCTA CCCCACATTA GGGTTTTCCT GGACAACAAT | 540 |
| CCCTGGGTCT GCGACTGCCA CATGGCAGAC ATGGTGACCT GGCTCAAGGA AACAGAGGTA | 600 |
| GTGCAGGGCA AAGACCGGCT CACCTGTGCA TATCCGGAAA AAATGAGGAA TCGGGTCCTC | 660 |
| TTGGAACTCA ACAGTGCTGA CCTGGACTGT GACCCGATTC TTCCCCCATC CCTGCAAACC | 720 |
| TCTTATGTCT TCCTGGGTAT TGTTTTAGCC CTGATAGGCG CTATTTTCCT CCTGGTTTTG | 780 |
| TATTTGAACC GCAAGGGGAT AAAAAAGTGG ATGCATAACA TCAGAGATGC CTGCAGGGAT | 840 |
| CACATGGAAG GGTATCATTA CAGATATGAA ATCAATGCGG ACCCCAGATT AACAAACCTC | 900 |
| AGTTCTAACT CGGATGTCCT CGAGTGA | |

Seq ID NO: 58 Protein sequence
Protein Accession #: NP_006661.1

| | |
|---|---|
| 1 11 21 31 41 51 | |
| \| \| \| \| \| \| | |
| MPGGCSRGPA AGDGRLRLAR LALVLLGWVS SSSPTSSASS FSSSAPFLAS AVSAQPPLPD | 60 |
| QCPALCECSE AARTVKCVNR NLTEVPTDLP AYVRNLFLTG NQLASNHFLY LPRDVLAQLP | 120 |
| SLRHLDLSNN SLVSLTYVSF RNLTHLESLH LEDNALKVLH NGTLAELQGL PHIRVFLDNN | 180 |
| PWVCDCHMAD MVTWLKETEV VQGKDRLTCA YPEKMRNRVL LELNSADLDC DPILPPSLQT | 240 |
| SYVFLGIVLA LIGAIFLLVL YLNRKGIKKW MHNIRDACRD HMEGYHYRYE INADPRLTNL | 300 |
| SSNSDVLE | |

Seq ID NO: 59 DNA sequence
Nucleic Acid Accession #: NM_024022
Coding sequence: 1-1362 (underlined sequences correspond to start and
stop codons)

| | |
|---|---|
| 1 11 21 31 41 51 | |
| \| \| \| \| \| \| | |
| ATGGGGGAAA ATGATCCGCC TGCTGTTGAA GCCCCCTTCT CATTCCGATC GCTTTTTGGC | 60 |
| CTTGATGATT TGAAAATAAG TCCTGTTGCA CCAGATGCAG ATGCTGTTGC TGCACAGATC | 120 |
| CTGTCACTGC TGCCATTGAA GTT1TfTCCA ATCATCGTCA TTGGGATCAT TGCATTGATA | 180 |
| TTAGCACTGG CCATTGGTCT GGGCATCCAC TTCGACTGCT CAGGGAAGTA CAGATGTCGC | 240 |
| TCATCCTTTA AGTGTATCGA GCTGATAGCT CGATGTGACG GAGTCTCGGA TTGCAAAGAC | 300 |
| GGGGAGGACG AGTACCGCTG TGTCCGGGTG GGTGGTCAGA ATGCCGTGCT CCAGGTGTTC | 360 |
| ACAGCTGCTT CGTGGAAGAC CATGTGCTCC GATGACTGGA AGGGTCACTA CGCAAATGTT | 420 |
| GCCTGTGCCC AACTGGGTTT CCCAAGCTAT GTGAGTTCAG ATAACCTCAG AGTGAGCTCG | 480 |
| CTGGAGGGGC AGTTCCGGGA GGAGTTTGTG TCCATCGATC ACCTCTTGCC AGATGACAAG | 540 |
| GTGACTGCAT TACACCACTC AGTATATGTG AGGGAGGGAT GTGCCTCTGG CCACGTGGTT | 600 |
| ACCTTGCAGT GCACAGCCTG TGGTCATAGA AGGGGCTACA GCTCACGCAT CGTGGGTGGA | 660 |
| AACATGTCCT TGCTCTCGCA GTGGCCCTGG CAGGCCAGCC TTCAGTTCCA GGGCTACCAC | 720 |
| CTGTGCGGGG GCTCTGTCAT CACGCCCCTG TGGATCATCA CTGCTGCACA CTGTGTTTAT | 780 |
| GACTTGTACC TCCCCAAGTC ATGGACCATC CAGGTGGGTC TAGTTTCCCT GTTGGACAAT | 840 |
| CCAGCCCCAT CCCACTGGGT GGAGAAGATT GTCTACCACA GCAAGTACAA GCCAAAGAGG | 900 |
| CTGGGCAATG ACATCGCCCT TATGAAGCTG GCCGGGCCAC TCACGTTCAA TGAAATGATC | 960 |
| CAGCCTGTGT GCCTGCCCAA CTCTGAAGAG AACTTCCCCG ATGGAAAAGT GTGCTGGACG | 1020 |
| TCAGGATGGG GGGCCACAGA GGATGGAGGT GACGCCTCCC CTGTCCTGAA CCACGCGGCC | 1080 |
| GTCCCTTTGA TTTCCAACAA GATCTGCAAC CACAGGGACG TGTACGGTGG CATCATCTCC | 1140 |
| CCCTCCATGC TCTGCGCGGG CTACCTGACG GGTGGCGTGG ACAGCTGCCA GGGGGACAGC | 1200 |
| GGGGGGCCCC TGGTGTGTCA AGAGAGGAGG CTGTGGAAGT TAGTGGGAGC GACCAGCTTT | 1260 |
| GGCATCGGCT GCGCAGAGGT GAACAAGCCT GGGGTGTACA CCCGTGTCAC CTCCTTCCTG | 1320 |
| GACTGGATCC ACGAGCAGAT GGAGAGAGAC CTAAAAACCT GA | |

Seq ID NO: 60 Protein sequence
Protein Accession #: NP_076927

| | |
|---|---|
| 1 11 21 31 41 51 | |
| \| \| \| \| \| \| | |
| MGENDPPAVE APFSFRSLFG LDDLKISPVA PDADAVAAQI LSLLPLKFFP ILVIGIIALI | 60 |
| LALAIGLGIH FDCSGKYRCR SSFKCIELIA RCDGVSDCKH GEDEYRCVRV GGQNAVLQVF | 120 |
| TAASWKTMCS DDWKGHYANV ACAQLGFPSY VSSDNLRVSS LEGQFRBEFV SIDHLLPDDK | 180 |
| VTALHHSVYV REGCASGHVV TLQCTACGHR RGYSSRIVGG NMSLLSQWPW QASLQFQGYH | 240 |
| LCGGSVITPL WIITAAHCVY DLYLPKSWTI QVGLVSLLDN PAPSHLVEKI VYHSKYKPKR | 300 |
| LGNDIALMKL AGPLTFNEMI QPVCLPNSEE NFPDGKVCWT SGWGATEDGG DASPVLNHAA | 360 |
| VPLISNKICN HRDVYGGIIS PSMLCAGYLT GGVDSCQGDS GGPLVCQERR LWKLVGATSF | 420 |
| GIGCAEVNKP GVYTRVTSFL DWIHEQMERD LKT | |

Seq ID NO: 61 DNA sequence
Nucleic Acid Accession #: NM_006475
Coding sequence: 28-2538 (underlined sequences correspond to start and
stop codons)

| | |
|---|---|
| 1 11 21 31 41 51 | |
| \| \| \| \| \| \| | |
| AACAGAACTG CAACGGAGAG ACTCAAGATG ATTCCCTTTT TACCCATGTT TTCTCTACTA | 60 |
| TTGCTGCTTA TTGTTAACCC TATAAACGCC AACAATCATT ATGACAAGAT CTTGGCTCAT | 120 |
| AGTCGTATCA GGGGTCGGGA CCAAGGCCCA AATGTCTGTG CCCTTCAACA GATTTTGGGC | 180 |
| ACCAAAAAGA AATACTTCAG CACTTGTAAG AACTGGTATA AAAAGTCCAT CTGTGGACAG | 240 |
| AAAACGACTG TTTTATATGA ATGTTGCCCT GGTTATATGA GAATGGAAGG AATGAAAGGC | 300 |
| TGCCCAGCAG TTTTGCCCAT TGACCATGTT TATGGCACTC TGGGCATCGT GGGAGCCACC | 360 |
| ACAACGCAGC GCTATTCTGA CGCCTCAAAA CTGAGGGAGG AGATCGAGGG AAAGGGATCC | 420 |
| TTCACTTACT TTGCACCGAG TAATGAGGCT TGGGACAACT TGGATTCTGA TATCCGTAGA | 480 |
| GGTTTGGAGA GCAACGTGAA TGITGAATTA CTGAATGCTT TACATAGTCA CATGATTAAT | 540 |
| AAGAGAATGT TGACCAAGGA CTTAAAAAAT GGCATGATTA TTCCTTCAAT GTATAACAAT | 600 |
| TTGGGGCTTT TCATTAACCA TTATCCTAAT GGGGTTGTCA CTGTTAATTG TGCTCGAATC | 660 |
| ATCCATGGGA ACCAGA1TGC AACAAATGGT GTTGTCCATG TCATTGACCG TGTGCTTACA | 720 |
| CAAATTGGTA CCTCAATTCA ATACTTCATT GAAGCAGAAG ATGACCTTTC ATCTTTTAGA | 780 |
| GCAGCTGCCA TCACATCGGA CATATTGGAG GCCCTTGGAA GAGACGGTCA CTTCACACTC | 840 |
| TTTGCTCCCA CCAATGAGGC TTTTGAGAAA CTTCCACGAG GTGTCCTAGA AAGGTTCATG | 900 |
| GGAGACAAAG TGGCTTCCGA AGCTCTTATG AAGTACCACA TCTTAAATAC TCTCCAGTGT | 960 |
| TCTGAGTCTA TTATGGGAGG AGCAGTCTTT GAGACGCTGG AAGGAAATAC AATTGAGATA | 1020 |
| GGATGTGACG GTGACAGTAT AACAGTAAAT GGAATCAAAA TGGTGAACAA AAAGGATATT | 1080 |
| GTGACAAATA ATGGTGTGAT CCATTTGATT GATCAGGTCC TAATTCCTGA TTCTGCCAAA | 1140 |
| CAAGTTATTG AGCTGGCTGG AAAACAGCAA ACCACCTTCA CGGATCTTGT GGCCCAATTA | 1200 |
| GGCTTGGCAT CTGCTCTGAG GCCAGATGGA GAATACACTT TGCTGGCACC TGTGAATAAT | 1260 |
| GCATTTTCTG ATGATACTCT CAGCATGGTT CAGCGCCTCC TTAAATTAAT TCTGCAGAAT | 1320 |
| CACATATTGA AAGTAAAAGT TGGCCTTAAT GAGCGTTACA ACGGGCAAAT ACTGGAAACC | 1380 |
| ATCGGAGGCA AACAGCTCAG AGTCTTCGTA TATCGTACAG CTGTCTGCAT TGAAAATTCA | 1440 |
| TGCATGGAGA AAGGGAGTAA GCAAGGGAGA AACGGTGCGA TTCACATATT CCGCGAGATC | 1500 |
| ATCAAGCCAG CAGAGAAATC CCTCCATGAA AAGTTAAAAC AAGATAAGCG CTTTAGCACC | 1560 |
| TTCCTCAGCC TACTTGAAGC TGCAGACTTG AAAGAGCTCC TGACACAACC TGGAGACTGG | 1620 |
| ACATTATTTG TGCCAACCAA TGATTGCTTT AAGGGAATGA CTAGTGAAGA AAAAGAAATT | 1680 |
| CTGATACGGG ACAAAAATGC TCTTCAAAAC ATCATTCTTT ATCACCTGAC ACCAGGAGTT | 1740 |
| TTCATTGGAA AAGGATTTGA ACCTGGTGTT ACTAACATTT TAAAGACCAC ACAAGGAAGC | 1800 |
| AAAATCTTTC TGAAAGAAGT AAATGATACA CTTCTGGTGA ATGAATTGAA ATCAAAAGAA | 1860 |
| TCTGACATCA TGACAACAAA TGGTGTAATT CATGTTGTAG ATAAACTCCT CTATCCAGCA | 1920 |
| GACACACCTG TTGGAAATGA TCAACTGCTG GAAATACTTA ATAAATTAAT CAAATACATC | 1980 |
| CAAATTAAGT TTGTTCGTGG TAGCACCTTC AAAGAAATCC CCGTGACTTT CTATACAACT | 2040 |
| AAAATTATAA CCAAAGTTGT GGAACCAAAA ATTAAAGTGA TTGAAGGCAG TCTTCAGCCT | 2100 |
| ATTATCAAAA CTGAAGGACC CACACTAACA AAAGTCAAAA TTGAAGGTGA ACCTGAATTC | 2160 |
| AGACTGATTA AAGAAGGTGA AACAATAACT GAAGTGATCC ATGGAGAGCC AATTATTAAA | 2220 |
| AAATACACCA AAATCATTGA TGGAGTGCCT GTGGAAATAA CTGAAAAAGA GACACGAGAA | 2280 |
| GAACGAATCA TTACAGGTCC TGAAATAAAA TACACTAGGA TTTCTACTGG AGGTGGAGAA | 2340 |
| ACAGAAGAAA CTCTGAAGAA ATTGTTACAA GAAGAGGTCA CCAAGGTCAC CAAATTCATT | 2400 |
| GAAGGTGGTG ATGGTCATTT ATTTGAAGAT GAAGAAATTA AAAGACTGCT TCAGGGAGAC | 2460 |
| ACACCCGTGA GGAAGTTGCA AGCCAACAAA AAAGTTCAAG GTTCTAGAAG ACGATTAAGG | 2520 |
| GAAGGTCGTT CTCAGTGAAA ATCCAAAAAC CAGAAAAAAA TGTTTATACA ACCCTAAGTC | 2580 |
| AATAACCTGA CCTTAGAAAA TTGTGAGAGC CAAGTTGACT TCAGGAACTG AAACATCAGC | 2640 |
| ACAAAGAAGC AATCATCAAA TAATTCTGAA CACAAATTTA ATATTTTTTT TTCTGAATGA | 2700 |
| GAAACATGAG GGAAATTGTG GAGTTAGCCT CCTGTGGTAA AGGAATfGAA GAAAATATAA | 2760 |
| CACCITACAC CCTTTTTCAT CTTGACATTA AAAGTTCTGG CTAACTTTGG AATCCATTAG | 2820 |
| AGAAAAATCC TTGTCACCAG ATTCATTACA ALTCAAATCG AAGAGTTGTG AACTGTfATC | 2880 |
| CCATTGAAAA GACCGAGCCT TGTATGTATG TTATGGATAC ATAAAATGCA CGCAAGCCAT | 2940 |
| TATCTCTCCA TGGGAAGCTA AGTTATAAAA ATAGGTGCTT GGTGTACAAA ACTTTTTATA | 3000 |
| TCAAAAGGCT TTGCACATTT CTATATGAGT GGGTTTACTG GTAAATTATG TTATTTTTTA | 3060 |
| CAACTAATTT TGTACTCTCA GAATGTTTGT CATATGCTTC TTGCAATGCA TATTTTTTAA | 3120 |
| TCTCAAACGT TTCAATAAAA CCATTTTTCA GATATAAAGA GAATTACTTC AAATTGAGTA | 3180 |
| ATTCAGAAAA ACTCAAGATT TAAGTTAAAA AGTGGTTTGG ACTTGGGAA | |

Seq ID NO: 62 Protein sequence:
Protein Accession #: NP_006466

| | |
|---|---|
| 1 11 21 31 41 51 | |
| \| \| \| \| \| \| | |
| MIPFLPMFSL LLLLIVNPIN ANNHYDKILA HSRIRGRDQG PNVCALQQIL GTKKKYFSTC | 60 |
| KNWYKKSICG QKTTVLYECC PGYMRMEGMK GCPAVLPIDH VYGTLGIVGA TTTQRYSDAS | 120 |
| KLREEIEGKG SFTYFAPSNE AWDNLDSDIR RGLESNVNVE LLNALHSHMI NKRMLTKDLK | 180 |
| NGMIIPSMYN NLGLFINHYP NGVVTVNCAR IIHGNQIATN GVVHVIDRVL TQIGTSIQDF | 240 |
| IEAEDDLSSF RAAAITSDIL EALGRDGHFT LFAPTNEAFE KLPRGVLERF MGDKVASEAL | 300 |
| MKYHILNTLQ CSESIMGGAV FETLEGNTIE IGCDGDSITV NGIKMVNKKD IVTNNGVIHL | 360 |
| IDQVLIPDSA KQVIELAGKQ QTTFTDLVAQ LGLASALRPD GEYTLLAPVN NAFSDDTLSM | 420 |
| VQRLLKLILQ NHILKVKVGL NELYNGQILE TIGGKQLRVE VYRTAVCIEN SCMEKGSKQG | 480 |
| RNGAIHIFRE IIKPAEKSLH EKLKQDKRFS TFLSLLEAAD LKELLTQPGD WTLFVPTNDA | 540 |
| FKGMTSEEKE ILIRDKNALQ NIILYHLTPG VFIGKGFEPG VTNILKTTQG SKIFLKEVND | 600 |
| TLLVNELKSK ESDIMTTNGV IHVVDKLLYP ADTPVGNDQL LEILNKLIKY IQIKFVRGST | 660 |
| FKEIPVTVYT TKIITKVVEP KIKVIEGSLQ PIIKTEGPTL TKVKIEGEPE ERLIKEGETI | 720 |
| TEVIHGEPII KKYTKIIDGV PVEITEKETR EERIITGPEI KYTRISTGGG ETEETLKKLL | 780 |
| QEEVTKVTKF IEGGDGHLFE DEEIKRLLQG DTPVRKLQAN KKVQGSRRRL REGRSQ | |

Seq ID NO: 63 DNA sequence
Nucleic Acid Accession #: NM_020974
Coding sequence: 81-3080 (underlined sequences correspond to start and
stop codons)

| | |
|---|---|
| 1 11 21 31 41 51 | |
| \| \| \| \| \| \| | |
| GGCGTCCGCG CACACCTCCC CGCGCCGCCG CCGCCACCGC CCGCACTCCG CCGCCTCTGC | 60 |
| CCGCAACCGC TGAGCCATCC ATGGGGGTCG CGGGCCGCAA CCGTCCCGGG GCGGCCTGGG | 120 |
| CGGTGCTGCT GCTGCTGCTG CTGCTGCCGC CACTGCTGCT GCTGGCGGGG GCCGTCCCGC | 180 |
| CGGGTCGGGG CCGTGCCGCG GGGCCGCAGG AGGATGTAGA TGAGTGTGCC CAAGGGCTAG | 240 |
| ATGACTGCCA TGCCGACGCC CTGTGTCAGA ACACACCCAC CTCCTACAAG TGCTCCTGCA | 300 |
| AGCCTGGCTA CCAAGGGGAA GGCAGGCAGT GTGAGGACAT CGATGAATGT GGAAATGAGC | 360 |
| TCAATGGAGG CTGTGTCCAT GACTGTTTGA ATATTCCAGG CAATTATCGT TGCACTTGTT | 420 |
| TTGATGGCTT CATGTTGGCT CATGACGGTC ATAATTGTCT TGATGTGGAC GAGTGCCTGG | 480 |
| AGAACAATGG CGGCTGCCAG CATACCTGTG TCAACGTCAT GGGGAGCTAT GAGTGCTGCT | 540 |
| GCAAGGAGGG GTTTTTCCTG AGTGACAATC AGCACACCTG CATTCACCGC TCGGAAGAGG | 600 |
| GCCTGAGCTG CATGAATAAG GATCACGGCT GTAGTCACAT CTGCAAGGAG GCCCCAAGGG | 660 |
| GCAGCGTCGC CTGTGAGTGC AGGCCTGGTT TTGAGCTGGC CAAGAACCAG AGAGACTGCA | 720 |
| TCTTGACCTG TAACCATGGG AACGGTGGGT GCCAGCACTC CTGTGACGAT ACAGCCGATG | 780 |
| GCCCAGAGTG CAGCTGCCAT CCACAGTACA AGATGCACAC AGATGGGAGG AGCTGCCTTG | 840 |
| AGCGAGAGGA CACTGTCCTG GAGGTGACAG AGAGCAACAC CACATCAGTG GTGGATGGGG | 900 |
| ATAAACGGGT GAAACGGCGG CTGCTCATGG AAACGTGTGC TGTCAACAAT GGAGGCTGTG | 960 |
| ACCGCACCTG TAAGGATACT TCGACAGGTG TCCACTGCAG GTGTCCTGTT GGATTCACTC | 1020 |
| TCCAGTfGGA TGGGAAGACA TGTAAAGATA TTGATGAGTG CCAGACCCGC AATGGAGGTT | 1080 |
| GTGATCATTT CTGCAAAAAC ATCGTGGGCA GTTTTGACTG CGGCTGCAAG AAAGGATTTA | 1140 |
| AATTATTAAC AGATGAGAAG TCTTGCCAAG ATGTGGATGA GTGCTCTTTG GATAGGACCT | 1200 |
| GTGACCACAG CTGCATCAAC CACCCTGGCA CATTTGCTTG TGCTTGCAAC CGAGGGTACA | 1260 |
| CCCTGTATGG CTTCACCCAC TGTGGAGACA CCAATGAGTG CAGCATCAAC AACGGAGGCT | 1320 |
| GTCAGCAGGT CTGTGTGAAC ACAGTGGGCA GCTATGAATG CCAGTGCCAC CCTGGGTACA | 1380 |
| AGCTCCACTG GAATAAAAAA GACTGTGTGG AAGTGAAGGG GCTCCTGCCC ACAAGTGTGT | 1440 |
| CACCCCGTGT GTCCCTGCAC TGCGGTAAGA GTGGTGGAGG AGACGGGTGC TTCCTCAGAT | 1500 |
| GTCACTCTGG CATTCACCTC TCTTCAGATG TCACCACCAT CAGGACAAGT GTAACCTTTA | 1560 |
| AGCTAAATGA AGGCAAGTGT AGTTTGAAAA ATGCTGAGCT GTTTCCCGAG GGTCTGCGAC | 1620 |
| CAGCACTACC AGAGAAGCAC AGCTCAGTAA AAGAGAGCTT CCGCTACGTA AACCTTACAT | 1680 |
| GCAGCTCTGG CAAGCAAGTC CCAGGAGCCC CTGGCCGACC AAGCACCCCT AAGGAAATGT | 1740 |
| TTATCACTGT TGAGTTTGAG CTTGAAACTA ACCAAAAGGA GGTGACAGCT TCTTGTGACC | 1800 |
| TGAGCTGCAT CGTAAAGCGA ACCGAGAAGC GGCTCCGTAA AGCCATCCGC ACGCTCAGAA | 1860 |
| AGGCCGTCCA CAGGGAGCAG TTTCACCTCC AGCTCTCAGG CATGAACCTC GACGTGGCTA | 1920 |
| AAAAGCCTCC CAGAACATCT GAACGCCAGG CAGAGTCCTG TGGAGTGGGC CAGGGTCATG | 1980 |
| CAGAAAACCA ATGTGTCAGT TGCAGGGCTG GGACCTATTA TGATGGAGCA CGAGAACGCT | 2040 |
| GCATTTTATG TCCAAATGGA ACCTTCCAAA ATGAGGAAGG ACAAATGAGT TGTGAACCAT | 2100 |
| GCCCAAGACC AGGAAATTCT GGGGCCCTGA AGACCCCAGA AGCITGGAAT ATGTCTGAAT | 2160 |
| GTGGAGGTCT GTGTCAACCT GGTGAATATT CTGCAGATGG CTTTGCACCT TGCCAGCTCT | 2220 |
| GTGCCCTGGG CACGTTCCAG CCTGAAGCTG GTCGAACTTC CTGCTTCCCC TGTGGAGGAG | 2280 |
| GCCYfGCCAC CAAACATCAG GGAGCTACTT CCTTTCAGGA CTGTGAAACC AGAGTTCAAT | 2340 |
| GTTCACCTGG ACATfTCTAC AACACCACCA CTCACCGATG TATTCGTTGC CCAGTGGGAA | 2400 |
| CATACCAGCC TGAATTTGGA AAAAATAATT GTGTGTCTTG CCCAGGAAAT ACTACGACTG | 2460 |
| ACTTTGATGG CTCCACAAAC ATAACCCAGT GTAAAAACAG AAGATGTGGA GGGGAGCTGG | 2520 |
| GAGATTTCAC TGGGTACATT GAATCCCCAA ACTACCCAGG CAATTACCCA GCCAACACCG | 2580 |
| AGTGTACGTG GACCATCAAC CCACCCCCCA AGCGCCGCAT CCTGATCGTG GTCCCTGAGA | 2640 |
| TCTTCCTGCC CATAGAGGAC GACTGTGGGG ACTATCTGGT GATGCGGAAA ACCTCTTCAT | 2700 |
| CCAATTCTGT GACAACATAT GAAACCTGCC AGACCTACGA ACGCCCCATC GCCTTCACCT | 2760 |
| CCAGGTCAAA GAAGCTGTGG ATTCAGTTCA AGTCCAATGA AGGGAACAGC GCTAGAGGGT | 2820 |
| TCCAGGTCCC ATACGTGACA TATGATGAGG ACTACCAGGA ACTCATTGAA GACATAGTTC | 2880 |
| GAGATGGCAG GCTCTATGCA TCTGAGAACC ATCAGGAAAT ACTTAAGGAT AAGAAACTTA | 2940 |
| TCAAGGCTCT GTTTGATGTC CTGGCCCATC CCCAGAACTA TTTCAAGTAC ACAGCCCAGG | 3000 |
| AGTCCCGAGA GATGTTTCCA AGATCGTTCA TCCGATTGCT ACGTTCCAAA GTGTCCAGGT | 3060 |
| TTTTGAGACC TTACAAATGA CTCAGCCCAC GTGCCACTCA ATACAAATGT TCTGCTATAG | 3120 |
| GGTTGGTGGG ACAGAGCTGT CTTCCTTCTG CATGTCAGCA CAGTCGGGTA TTGCTGCCTC | 3180 |
| CCGTATCAGT GACTCATTAG AGTTCAATTT TTATAGATAA TACAGATATT TTGGTAAATT | 3240 |
| GAACTTGGTT TTTCTTTCCC AGCATCGTGG ATGTAGACTG AGAATGGCTT TGAGTGGCAT | 3300 |
| CAGCTTCTCA CTGCTGTGGG CGGATGTCTT GGATAGATCA CGGGCTGGCT GAGCTGGACT | 3360 |
| TTGGTCAGCC TAGGTGAGAC TCACCTGTCC TTCTGGGGTC TTACTCCTCC TCAAGGAGTC | 3420 |
| TGTAGTGGAA AGGAGGCCAC AGAATAAGCT GCTTATTCTG AAACTTCAGC TTCCTCTAGC | 3480 |
| CCGGCCCTCT CTAAGGGAGC CCTCTGCACT CGTGTGCAGG CTCTGACCAG GCAGAACAGG | 3540 |
| CAAGAGGGGA GGGAAGGAGA CCCCTGCAGG CTCCCTCCAC CCACCTTGAG ACCTGGGAGG | 3600 |
| ACTCAGTTTC TCCACAGCCT TCTCCAGCCT GTGTGATACA AGTTTGATCC CAGGAACTTG | 3660 |
| AGTTCTAAGC AGTGCTCGTG AAAAAAAAAA GCAGAAAGAA TTAGAAATAA ATAAAAACTA | 3720 |
| AGCACTTCTG GAGACAT | |

Seq ID NO: 64 Protein sequence:
Protein Accession #: NP_066025.1

| | |
|---|---|
| 1 11 21 31 41 51 | |
| \| \| \| \| \| \| | |
| MGVAGRNRPG AAWAVLLLLL LLPPLLLLAG AVPPGRGPAA GPQEDVDECA QGLDDCHADA | 60 |
| LCQNTPTSYK CSCKPGYQGE GRQCEDIDEC GNELNGGCVH DCLNIPGNYR CTCFDGFMLA | 120 |
| HDGHNCLDVD ECLENNGGCQ HTCVNVMGSY ECCCKEGFFL SDNQHTCIHR SEEGLSCMNK | 180 |
| DHGCSHICKE APRGSVACEC RPGFELAKNQ RDCILTCNHG NGGCQHSCDD TADGPECSCH | 240 |
| PQYKMHTDGR SCLEREDTVL EVTESNTTSV VDGDKRVKRR LLMETCAVNN GGCDRTCKDT | 300 |
| STGVHCSCPV GFTLQLDGKT CKDIDECQTR NGGCDTHCKN IVGSFDCGCK KGFKLLTDEK | 360 |
| SCQDVDECSL DRTCDHSCIN HPGTFACACN RGYTLYGFTH CGDTNECSIN NGGCQQVCVN | 420 |
| TVGSYECQCH PGYKLHWNKK DCVEVKGLLP TSVSPRVSLH CGKSGGGDGC FLRCHSGIHL | 480 |
| SSDVTHIRTS VTFKLNEGKC SLKNAELFPE GLRPALPEKH SSVKESFRYV NLTCSSGKQV | 540 |
| PGAPGRPSTP KEMFITVEFE LETNQKEVTA SCDLSC1VKR TEKRLRKAIR TLRKAVHREQ | 600 |
| FHLQLSGMNL DVAKKPPRTS ERQAESCGVG QGHAENQCVS CRAGTYYDGA RERCILCPNG | 660 |
| TFQNEEGQMT CEPCPRPGNS GALKTPEAWN MSECGGLCQP GEYSADGFAP CQLCALGTFQ | 720 |
| PEAGRTSCFP CGGGLATKHQ GATSFQDCET RVQCSPGHFY NTTTHRCIRC PVGTYQPEFG | 780 |
| KNNCVSCPGN TTTDFDGSTN ITQCKNRRCG GELGDFTGYI ESPNYPGNYP ANTECTWT1N | 840 |
| PPPKRRILTV VPEIFLPIED DCGDYLVMRX TSSSNSVTTY ETCQTYERPI AFTSRSKKLW | 900 |
| IQFKSNEGNS ARGFQVPYVT YDEDYQELIE DIVRDGRLYA SENHQEILKD KKLIKALFDV | 960 |
| LAHPQNYFKY TAQESREMEP RSFIRLLRSK VSRFLRPYK | |

Seq ID NO: 65 DNA sequence
Nucleic Acid Accession #: NM_007210
Coding sequence: 1-1869 (underlined sequences correspond to start and
stop codons)

| | |
|---|---|
| 1 11 21 31 41 51 | |
| \| \| \| \| \| \| | |
| ATGAGGCTCC TCCGCAGACG CCACATGCCC CTGCGCCTGG CCATGGTGGG CTGCGCCTTT | 60 |
| GTGCTCTTCC TCTTCCTCCT GCATAGGGAT GTGAGCAGCA GAGAGGAGGC CACAGAGAAG | 120 |
| CCGTGGCTGA AGTCCCTGGT GAGCCGGAAG GATCACGTCC TGGACCTCAT GCTGGAGGCC | 180 |
| ATGAACAACC TTAGAGATTC AATGCCCAAG CTCCAAATCA GGGCTCCAGA AGCCCAGCAG | 240 |
| ACTCTGTTCT CCATAAACCA GTCCTGCCTC CCTGGGTTCT ATACCCCAGC TGAACTGAAG | 300 |
| CCCTTCTGGG AACGGCCACC ACAGGACCCC AATGCCCCTG GGGCAGATGG AAAAGCATTT | 360 |
| CAGAAGAGCA AGTGGACCCC CCTGGAGACC CAGGAAAAGG AAGAAGGCTA TAAGAAGCAC | 420 |
| TGTTTCAATG CCTTTGCCAG CGACCGGATC TCCCTGCAGA GGTCCCTGGG GCCAGACACC | 480 |
| CGACCACCTG AGTGTGTGGA CCAGAAGTTC CGGCGCTGCC CCCCACTGGC CACCACCAGC | 540 |
| GTGATCATTG TGTTCCACAA CGAAGCCTGG TCCAGACTGC TGCGAACAGT GTACAGCGTC | 600 |
| CTACACACCA CCCCTGCCAT CTTGCTCAAG GAGATCATAC TGGTGGATGA TGCCAGCACA | 660 |
| GAGGAGCACC TAAAGGAGAA GCTGGAGCAG TACGTGAAGC AGCTGCAGGT GGTGAGGGTG | 720 |
| GTGCGGCAGG AGGAGCGGAA GGGGTTGATC ACCGCCCGGC TGCTGGGGGC CAGCGTGGCA | 780 |
| CAGGCGGAGG TGCTCACGTT CCTGGATGCC CACTGTGAGT GCTTCCACGG CTGGCTGGAG | 840 |
| CCCCTCCTGG CTCGAATCGC TGAGGACAAG ACAGTGGTGG TGAGCCCAGA CATCGTCACC | 900 |
| ATCGACCTfA ATACTTTTGA GTTCGCCAAG CCCGTCCAGA GGGGCAGAGT CCATAGCCGA | 960 |
| GGCAACTTTG ACTGGAGCCT GACCTTCGGC TGGGAAACAC TTCCTCCACA TGAGAAGCAG | 1020 |
| AGGCGCAAGG ATGAAACATA CCCCATCAAA TCCCCGACGT TTGCTGGTGG CCTCTTCTCC | 1080 |
| ATCCCCAAGT CCTACTTTGA GCACATCGGT ACCTATGATA ATCAGATGGA GATCTGGGGA | 1140 |
| GGGGAGAACG TGGAAATGTC CTTCCGGGTG TGGCAGTGTG GGGGCCAGCT GGAGATCATC | 1200 |
| CCCTGCTCTG TCGTAGGCCA TGTGTTCCGG ACCAAGAGCC CCCACACCTT CCCCAAGGGC | 1260 |
| ACTAGTGTCA TTGCTCGCAA TCAAGTGCGC CTGGCAGAGG TCTGGATGGA CAGCTACAAG | 1320 |
| AAGATTTTCT ATAGGAGAAA TCTGCAGGCA GCAAAGATGG CCCAAGAGAA ATCCTTCGGT | 1380 |
| GACATTTCGG AACGACTGCA GCTGAGGGAA CAACTGCACT GTCACAACTT TTCCTGGTAC | 1440 |
| CTGCACAATG TCTACCCAGA GATGTTTGTT CCTGACCTGA CGCCCACCTT CTATGGTGCC | 1500 |
| ATCAAGAACC TCGGCACCAA CCAATGCCTG GATGTGGGTG AGAACAACCG CGGGGGGAAG | 1560 |
| CCCCTCATCA TGTACTCCTG CCACGGCCTT GGCGGCAACC AGTACTTTGA GTACACAACT | 1620 |
| CAGAGGGACC TTCGCCACAA CATCGCAAAG CAGCTGTGTC TACATGTCAG CAAGGGTGCT | 1680 |
| CTGGGCCTTG GGAGCTGTCA CTTCACTGGC AAGAATAGCC AGGTCCCCAA GGACGAGGAA | 1740 |
| TGGGAATTGG CCCAGGATCA GCTCATCAGG AACTCAGGAT CTGGTACCTG CCTGACATCC | 1800 |
| CAGGACAAAA AGCCAGCCAT GGCCCCCTGC AATCCCAGTG ACCCCCATCA GTTGTGGCTC | 1860 |
| TTTGTCTAGG ACCCAGATCA TCCCCAGAGA GAGCCCCCAC AAGCTCCTCA GGAAACAGGA | 1920 |
| TTGCTGATGT CTGGGAACCT GATCACCAGC TTCTCTGGAG GCCGTAAAGA TGGAT3TCTA | 1980 |
| AACCCACTGG GTGGCAAGGC AGGACCTTCC TAATCCTTGC AACAACATTG GGCCCATTTT | 2040 |
| CTTTCCTTCA CACCGATGGA AGAGACCATT AGGACATATA TTTAGCCTAG CGTTTTCCTG | 2100 |
| TTCTAGAAAT AGAGGCTCCC AAAGTAGGGA AGGCAGCTGG GGGAGGGTTC AGGGCAGCAA | 2160 |
| TGCTGAGTTC AAGAAAAGTA CTTCAGGCTG GGCACAGTGG CTCATGCCTG AAATCCTAGC | 2220 |
| ACTTTGGGAA GACAATGTGG GAGAATGGCT TGAGCCCAGG AGTTCAAGAC CGGCCTGAGC | 2280 |
| AACATAGTGA GGATCCCATC TCTACGCCCA CCCTCCCCCC GGCAAAAAAA AAAGCTGGGT | 2340 |
| ATGGTGGCTT ATGCCTGTAG TCGCAGCTAC TCAGAAGGCT GAGGTGGGAG GATTGCTTGT | 2400 |
| TCCCCGGAGG TTGAAGCTAC AGTGAGCCTT GATTGTGTCA CTGCACTCCA GCCTGGGCAA | 2460 |
| CAGGTAAGAC TCTGTCTCAA AAAAAAAACA AAAAAGAAGA AGAAAAGTAC TTCTACAGCC | 2520 |
| ATGTCCTATT CCTTGATCAT CCAAAGCACC TGCAGAGTCC AGTGAAATGA TATATTCTGG | 2580 |
| CTGGGCACAG TGGCTCACAC CTGTAATCCT AGCACTTTGG GAGGCCAAGG CAGGTGGATC | 2640 |
| ACCTGAGGTC AGAAGTTTGA AACCAGCCTG GACTACATGG TGAAACTCCA TCTCTACTAA | 2700 |
| AAGTACAAAA ATTAGCTGGG CATGATGGCA CGCACCTGCA GTCCCAGCTA CTTGGGAGGC | 2760 |
| TGAGGCAGGA GAATCACTCG AACCCAGGAG GCAGAGGTTG CAGTGAGCCA AGACAGCACC | 2820 |
| ATTGCACCCC AGCCTGAGCA ACAAGAGCGA AACTCCATCT CAGGAAAAAA AAAAAAAAAA | 2880 |
| A | |

Seq ID NO: 66 Protein sequence:
Protein Accession #: NP_009141

| | |
|---|---|
| 1 11 21 31 41 51 | |
| \| \| \| \| \| \| | |
| MRLLRRPHMP LRLAMVGCAF VLFLFLLHRD VSSRBEATEK PWLKSLVSRX DHVLDLMLEA | 60 |
| MNNLRDSMPK LQIRAPEAQQ TLFSINQSCL PGFYTPAELK PEWERPPQDP NAPGADGKAF | 120 |
| QKSKWTPLET QEKEEGYKKH CFNAFASDRI SLQRSLGPDT RPPECVDQKF RRCPPLATTS | 180 |
| VIIVFHNEAW STLLRTVYSV LHTTPAILLK EIILVDDAST EEHLKEKLEQ YVKQLQVVRV | 240 |
| VRQEERKGLI TARLLGASVA QAEVLTFLDA HCECFHGWLE PLLARIAEDK TVVVSPDIVT | 300 |
| IDLNTFEFAK PVQRGRVHSR GNFDWSLTFG WETLPPHEKQ RRXDETYPIK SPTFAGGLFS | 360 |
| LPKSYFEHIG TYDNQMEIWG GENVEMSFRV WQCGGQLEII PCSVVGHVFR TKSPHTFPKG | 420 |
| TSVIARNQVR LAEVWMDSYK KIFYRRNLQA AXMAQEKSFG DISERLQLRB QLHCHNFSWY | 480 |
| LHNVYPEMFV PDLTPTFYGA IKNLGTNQCL DVGENNRGGK PLIMYSCHGL GGNQYFEYTT | 540 |
| QRDLRGNIAX QLCLHVSKGA LGLGSCHFTG GNSQVPKDEE WELAQDQLIR NSGSGTCLTS | 600 |
| QDKKPAMAPC NPSDPHQLWL FV | |

Seq ID NO: 67 DNA sequence
Nucleic Acid Accession #: NM_014112
Coding sequence: 600-4484 (underlined sequences correspond to start
and stop codons)

| | |
|---|---|
| 1 11 21 31 41 51 | |
| \| \| \| \| \| \| | |
| TTCCTCCGCG AAGGCTCCTT TGATATTAAT AGTGTTGGTG TCTTGAAACT GACGTAATGC | 60 |
| GCGGAGACTG AGGTCCTGAC AAGCGATAAC ATTTCTGATA AAGACCCGAT CTTACTGCAA | 120 |
| TCTCTAGCGT CCTCTTTTTT GGTGCTGCTG GTTTCTCCAG ACCTCGCGTC CTCTCGATTG | 180 |
| CTCTCTCGCC FTCCTATTTC TTTTTITTTT TTTTAAACAA AAAACAACAC CCCCTCCCCT | 240 |
| CTCCCACCCG GCACCGGGCA CATCCTTGCT CTATTTCCTT TCTCTTTCTC TCTCTCTCTC | 300 |
| TCTCTTTTTT AATAAGGGTG GGGGAGGGAA AGGGGGGGGA GGCAGGAAAG ACCTTTTTCT | 360 |
| CTCCCCCCCG CAATAATCCA AGATCAACTC TGCAAACAAC AGAAGACGGT TCATGGCTTT | 420 |
| GGCCGCCGCG CCACCATCTT TCGGGCTGCC GAGGGTGTTC TTGACGATTA ATCAACAGAT | 480 |
| GTACAGATCA GCTCTCAAAA TGTCTTCTGT GTCTTCTGAG CGTCTTCTAA GACAATTGCA | 540 |
| TTAGCCTCCT GCTAGTTGAC TAATAGAATT AATAATTGTA AAAAGCACTC TAAAGCCACA | 600 |
| TGCCTTATGA AGTCAATGCT GGGTATGATT TTACAAATAT GGTCCGGAAA AAGAACCCCC | 660 |
| CTCTGAGAAA CGTTGCAAGT GAAGGCGAGG GCCAGATCCT GGAGCCTATA GGTACAGAAA | 720 |
| GCAAGGTATC TGGAAAGAAC AAAGAATTCT CTGCAGATCA GATGTCAGAA AATACGGATC | 780 |
| AGAGTGATGC TGCAGAACTA AATCATAAGG AGGAACATAG CTTGCATGTT CAAGATCCAT | 840 |
| CTTCTAGCAG TAAGAAGGAC TTGAAAAGCG CAGTTCTGAG TGAGAAGGCT GGCTTCAATT | 900 |
| ATGAAAGCCC CAGTAAGGGA GGAAACTTTC CCTCCTTTCC GCATGATGAG GTGACAGACA | 960 |
| GAAATATGTT GGCTTTCTCA TTTCCAGCTG CTGGGGGAGT CTGTGAGCCC TTGAAGTCTC | 1020 |
| CGCAAAGAGC AGAGGCAGAT GACCCTCAAG ATATGGCCTG CACCCCCTCA GGGGACTCAC | 1080 |
| TGGAGACAAA GGAAGATCAG AAGATGTCAC CAAAGGCTAC AGAGGAAACA GGGCAAGCAC | 1140 |
| AGAGTGGTCA AGCCAATTGT CAAGGTTTGA GCCCAGTTTC AGTGGCCTCA AAAAACCCAC | 1200 |
| AAGTGCCTTC AGATGGGGGT GTAAGACTGA ATAAATCCAA AACTGACTTA CTGGTGAATG | 1260 |
| ACAACCCAGA CCCGGCACCT CTGTCTCCAG AGCTTCAGGA CTTTAAATGC AATATCTGTG | 1320 |
| GATATGGTTA CTACGGCAAC GACCCCACAG ATCTGATTAA GCACTTCCGA AAGTATCACT | 1380 |
| TAGGACTGCA TAACCGCACC AGGCAAGATG CTGAGCTGGA CAGCAAAATC TTGGCCCTTC | 1440 |
| ATAACATGGT GCAGTTCAGC CATTCCAAAG ACTTCCAGAA GGTCAACCGT TCTGTGTTTT | 1500 |
| CTGGTGTGCT GCAGGACATC AATTCTTCAA GGCCTGTTTT ACTAAATGGG ACCTATGATG | 1560 |
| TGCAGGTGAC TTCAGGTGGA ACATTCATTG GCATTGGACG GAAAACACCA GATTGCCAAG | 1620 |
| GGAACACCAA GTATTTCCGC TGTAAATTCT GCAATTTCAC TTATATGGGC AACTCATCCA | 1680 |
| CCGAATTAGA ACAACATTTT CTTCAGACTC ACCCAAACAA AATAAAAGCT TCTCTCCCCT | 1740 |
| CCTCTGAGGT TGCAAAACCT TCAGAGAAAA ACTCTAACAA GTCCATCCCT GCACTTCAAT | 1800 |
| CCAGTGATTC TGGAGACTTG GGAAAATGGC AGGACAAGAT AACAGTCAAA GCAGGAGATG | 1860 |
| ACACTCCTGT TGGGTACTCA GTGCCCATAA AGCCCCTCGA TTCCTCTAGA CAAAATGGTA | 1920 |
| CAGAGGCCAC CAGTTACTAC TGGTGTAAAT TTTGTAGTTT CAGCTGTGAG TCATCTAGCT | 1980 |
| CACTTAAACT GCTAGAACAT TATGGCAAGC AGCACGGAGC AGTGCAGTCA GGCGGCCTTA | 2040 |
| ATCCAGAG1T AAATGATAAG CITTCCAGGG GCTCTGTCAT TAATCAGAAT GATCTAGCCA | 2100 |
| AAAGTTCAGA AGGAGAGACA ATGACCAAGA CAGACAAGAG CTCGAGTGGG GCTAAAAAGA | 2160 |
| AGGACTTCTC CAGCAAGGGA GCCGAGGATA ATATGGTAAC GAGCTATAAT TGTCAGTTCT | 2220 |
| GTGACTTCCG ATATTCCAAA AGCCATGGCC CTGATGTAAT TGTAGTGGGG CCACTTCTCC | 2280 |
| GTCATTATCA ACAGCTCCAT AACATTCACA AGTGTACCAT TAAACACTGT CCATTCTGTC | 2340 |
| CCAGAGGACT TTGCAGCCCA GAAAAGCACC TTGGAGAAAT TACTTATCCG TTTGCTTGTA | 2400 |
| GAAAAAGTAA TTGTTCCCAC TGTGCACTCT TGCTTCTGCA CTTGTCTCCT GGGGCGGCTG | 2460 |
| GAAGCTCGCG AGTCAAACAT CAGTGCCATC AGTGTTCATT CACCACCCCT GACGTAGATG | 2520 |
| TACTCCTCTT TCACTATGAA AGTGTGCATG AGTCCCAATC ATCGGATGTC AAACAAGAAG | 2580 |
| CAAATCACCT GCAAGGATCG GATGGGCAGC AGTCTTTCAA GGAAAGCAAA GAACACTCAT | 2640 |
| GTACCAAATG TGATTTTATT ACCCAAGTGG AAGAAGAGAT TTCCCGACAC TACAGGAGAG | 2700 |
| CACACAGCTG CTACAAATGC CGTCAGTGCA GTTTTACAGC TGCCGATACT CAGTCACTAC | 2760 |
| TGGAGCACTT CAACACTGTT CACTGCCAGG AACAGGACAT CACTACAGCC AACGGCGAAG | 2820 |
| AGGACGGTCA TGCCATATCC ACCATCAAAG AGGAGCCCAA AATTGACTTC AGGGTCTACA | 2880 |
| ATCTGCTAAC TCCAGACTCT AAAATGGGAG AGCCAGTTTC TGAGAGTGTG GTGAAGAGAG | 2940 |
| AGAAGCTGGA AGAGAAGGAC GGGCTCAAAG AGAAAGTTTG GACCGAGAGT TCCAGTGATG | 3000 |
| ACCTTCGCAA TGTGACTTGG AGAGGGGCAG ACATCCTGCG GGGGAGTCCG TCATACACCC | 3060 |
| AAGCAAGCCT GGGGCTGCTG ACGCCTGTGT CTGGCACCCA AGAGGAGACA AAGACTCTAA | 3120 |
| GGGATAGTCC CAATGTGGAG GCCGCCCATC TGGCGCGACC TATTTATGGC TTGGCTGTGG | 3180 |
| AAACCAAGGG ATTCCTGCAG GGGGCGCCAG CTGGCGGAGA GAAGTCTGGG GCCCTCCCCC | 3240 |
| AGCAGTATCC TGCATCGGGA GAAAACAAGT CCAAGGATGA ATCCCAGTCC CTGTTACGGA | 3300 |
| GGCGTAGAGG CTCCGGTGTT TTTTGTGCCA ATTGCCTGAC CACAAAGACC TCTCTCTGGC | 3360 |
| GAAAGAATGC AAATGGCGGA TATGTATGCA ACGCGTGTGG CCTCTACCAG AAGCTTCACT | 3420 |
| CGACTCCCAG GCCTTTAAAC ATCATTAAAC AAAACAACGG TGAGCAGATT ATTAGGAGGA | 3480 |
| GAACAAGAAA GCGCCTTAAC CCAGAGGCAC TTCAGGCTGA GCAGCTCAAC AAACAGCAGA | 3540 |
| GGGGCAGCAA TGAGGAGCAA GTCAATGGAA GCCCGTTAGA GAGGAGGTCA GAAGATCATC | 3600 |
| TAACTGAAAG TCACCAGAGA GAAATTCCAC TCCCCAGCCT AAGTAAATAC GAAGCCCAGG | 3660 |
| GTTCATTGAC TAAAAGCCAT TCTGCTCAGC AGCCAGTCCT GGTCAGCCAA ACTCTGGATA | 3720 |
| TTCACAAAAG GATGCAACCT TTGCACATTC AGATAAAAAG TCCTCAGGAA AGTACTGGAG | 3780 |
| ATCCAGGAAA TAGTTCATCC GTATCTGAAG GGAAAGGAAG TTCTGAGAGA GGCAGTCCTA | 3840 |
| TAGAAAAGTA CATGAGACCT GCGAAACACC CAAATTATTC ACCACCAGGC AGCCCTATTG | 3900 |
| AAAAGTACCA GTACCCACTT TTTGGACTTC CCTTTGTACA TAATGACTTC CAGAGTGAAG | 3960 |
| CTGATTGGCT GCGGTTCTGG AGTAAATATA AGCTCTCCGT TCCTGGGAAT CCGCACTACT | 4020 |
| TGAGTCACGT GCCTGGCCTA CCAAATCCTT GCCAAAACTA TGTGCCTTAT CCCACCTTCA | 4080 |
| ATCTGCCTCC TCATTTTTCA GCTGTTGGAT CAGACAATGA CATTCCTCTA GATTTGGCGA | 4140 |
| TCAAGCATTC CAGACCTGGG CCAACTGCAA ACGGTGCCTC CAAGGAGAAA ACGAAGGCAC | 4200 |
| CACCAAATGT AAAAAATGAA GGTCCCTTGA ATGTAGTAAA AACAGAGAAA GTTGATAGAA | 4260 |
| GTACTCAAGA TGAACTTTCA ACAAAATGTG TGCACTGTGG CATTGTCTTT CTGGATGAAG | 4320 |
| TGATGTATGC TTTGCATATG AGTTGCCATG GTGACAGTGG ACCTTTCCAG TGCAGCATAT | 4380 |
| GCCAGCATCT TTGCACGGAC AAATATGACT TCACAACACA TATCCAGAGG GGCCTGCATA | 4440 |
| GGAACAATGC ACAAGTGGAA AAAAATGGAA AACCTAAAGA GTAAAACCTT AGCACTTAGC | 4500 |
| ACAATTAAAT AGAAATAGGT TTTCTTGATG GGAATTCAAT AGCTTGTAAT GTCTTATGAA | 4560 |
| GACCTATTAA AAAAATACTT CATAGAGCCT GCCTTATCCA ACATGAAATT CCCTTCTTTT | 4620 |
| GTTATTCTTT CTTTTGATGA GTAGGTTACC AAGATTAAAA AGTGAGATAA ATGGTCAATG | 4680 |
| AGAAAGAATG GAAGATGGTA AACAATCACT TTTTAAAACC TGTTAAGTCA AAACCATCIT | 4740 |
| GGCTAATATG TACTGGGGAA ATAATCCATA AGAGATATCA CCAGACTAGA ATTAATATAT | 4800 |
| TfATAAAGAA AGAGACCAAA ACTGTCTAGA ATTTGAAAGG GTTTACATAT TATTATACTA | 4860 |
| AAGCAGTACT GGACTGGCCA TTGGACCATT TGTTCCAAAA CCCATAAATT GTTGCCTAAA | 4920 |
| TTTATAATGA TCATGAAACC CTAGGCAGAG GAGGAGAAAT TGAAGGTCCA GGGCAATGAA | 4980 |
| AGAAAAATGG CGCCCTCTCA ATTTAGTCTT CTCTCATTGG CCATGTTTCA GATTTTGACC | 5040 |
| TAGAAATGCG AGCTGTGGTT AGGCTTGGTT AGAGTGCAGC AAGCAACATG ACAGATGGTG | 5100 |
| GCACGCTGTT TTTACCCAGC CCTGCCTGTA CATACACATG CACACCCTCT CTGATATTTT | 5160 |
| TGTCCTTTAG ATGTTCAAAT ACTCAGTAGT CCTTTTGTTT GCGGTTTAGA TTCATTTTGT | 5220 |
| CCACACATGT ACCCATTTTA AAAAACAATG TCCTCGATGC TTCTGTAGTG ATTTCATTTT | 5280 |
| AGCCAGGTAT TTCTTTCTTG TGTGTGATGA ACCAGTATGG ATTTGCTTTT CTAAGCCTCC | 5340 |
| TGTTGGTTAC TAATCTCACT TGGCACATTA TAACTAAAGG AATCCCCTCA ATTCAAAAGC | 5400 |
| ATAGATGGAT ACAAATGTCA GACCGTGGGT TTAATTTGTT TAGAACACAT GGCATTTCTT | 5460 |
| CACAAGGTAA CCTGCTGTAT TTATTTATTT TCTTTTGGTT AAATATAATT TCCAAACTTT | 5520 |
| GTGGTCAGGC AGCGTCTAAG GTTACGTTAC CACAGACTGA CAGTTGGTAT ATGTACCAGC | 5580 |
| CAATCCCTTC ATTAAATGTA TACAGATTTA GTTAAGTAGC ATTAAATAGG ATTCTTAGAA | 5640 |
| GTATGTCCTG ATAGAACTTT TAATACTTAA GGCTTTGTAA AAACTATCCA TGAAGGGAAA | 5700 |
| GCTCCTCAGC ATAACTGCTC AGGGAAATAG GGCTAAATAA CTGAACATTA AATAATTGGT | 5760 |
| TAAAGGTGCT GTTAGTCGAG CCTCAATGCT TGCTACAAGG ATGTATGTAC AAGGACTGAC | 5820 |
| TTTAATAATT TGCATTATAT TGTCCCAACC AGTAGTTTAT TTI1TGCCAC GGAGATGTAG | 5880 |
| AAGATATTAC AAGCTACTGG ATGCACTGTC AGATTAACTT ATTTCATTAA AGAAGTTGGG | 5940 |
| AGAACAAATA GGAAAAAAAA AACTTATTTT TCTAGTAAAT ATTAATGTAT TACATTTCAA | 6000 |
| ATAATGGTGC CTGACATATT GAATAATTAT TTTCTACAGT GTACGTATGC AACAAAGATA | 6060 |
| TTCCATCATG CATTAGAGTC AGTTCTGGCT CTGCCTAGCT GTTTACATTT GCAAATGTAG | 6120 |
| CAAACAAGGT AATGAAGCAA CTATHTCTAT TGCAGTAGAT ATCCTHTTGT GTGTGTGTGT | 6180 |
| GTGCATTAAA GTTGTAAACG GTAACATGAA ACAAATGAAA GTTCTTGCTA TAATGGTATG | 6240 |
| GAAAACAAGA AGGAAATGAA AATATTTTTA TGCCTACTTA GGAAAAAAAG GGTAGCACIT | 6300 |
| ATTCATTCCA AGTACTTTTT TTTTTTTAAT TTTTAAGCTC TTAACTCACA TTGTTATGCT | 6360 |
| TAAGATGATA AACATATATC CTCTTTTTAT TGCTITGTCT ATGTTTCATA TGAAACATTT | 6420 |
| CAGAAATTAT TTTGATAAGT GTTGCTGGAA TCTGCAACGC TGATTTTTTT TTGCATTCTG | 6480 |
| TAGTCGCATT TGCACTCCAT TTTTACATTA ATTCGCAGTT GCTTTGTATC ATTGTTTTGT | 6540 |
| TTGGGTTTTG TITCTTTTTC ACAGTGCCGG GTCTTCGTTT CTTAAAGTTG GATGGCAGGT | 6600 |
| AGAGTTCAAC CAGTTCGTGA CTGTTGTAGC GAATGAAGTT AAAAAAATGT CTTTCTGATG | 6660 |
| TTGTGTTGTC ATTTTCATTT TTGCATTTTT TfGTTTGCAT ATTAAAAAAA GAGAAAAGAG | 6720 |
| AAAGCAAGAG ACAGAAATCA GGACTAAGTC CTCTGCTTCA GTTTCATTGT TAACGGGCCT | 6780 |
| TATTCTGATC TCACCTGTCG CGTAGCTCTA ATATTCACAT AAACTGAAAT AAAGAAGTGG | 6840 |
| AATGAGGAGC TITGACATTC AAATTATGTG ATGTAATTTA TCTTCCTTAG GAATTTTGAT | 6900 |
| GGATGCATCT CAAAATGTAT AGCCAGACTT GAGAGGTGAC AATTAAAGAT CTAAAAAAGA | 6960 |
| GAGGAGATTC CCCCAAACAA CAATATTTAA TTTTCTTAGT AAAAAGAATA ACAGAATGCA | 7020 |
| TCGTGGCAAT CCTTAAGCAA CATTATCTAT GTGGACTGCT TAAATCAGCA AAACACCAGA | 7080 |
| AGTTTGGTTA ACTTGGGCAA TATGACAAGT ATTACTTTTT GGGCAAAACT ACTCATTAAG | 7140 |
| CAATTTCTCT AGTGTGTCGG ACACAAATAG GTTCTTTATT TTTGGCATGT ATGCCTTTTT | 7200 |
| ATTTTCATTC AATTYTTTTT TTTTCTCAGA CAGACATAGT AGTATCAACT AGCATTGGAA | 7260 |
| AATACATATC ACTATTCTTG GAATATTTAT GGTCAGTCTA CTTTTTAGTA AAATATTTTT | 7320 |
| GGATAGCGTT GACAGGATAG ATCTTTATCC ATAGTTCITT ATTATTGATA ATTTTATTTT | 7380 |
| CATTTTTTGC TTTCATTATT ATACATATTT TGGTGGAGAA GAGGTTGGGC TTTTTTGAAA | 7440 |
| GAGACAAAAA TTTATTATAA CACTAAACAC TCCTTTTTTG ACATATTAAA GCCTTTATTC | 7500 |
| CATCTCTCAA GATATATTAT AAAATTTATT TTTTTAATTT AAGATTTCTG AATTATTTTA | 7560 |
| TCTTAAATTG TGATTTTAAA CGAGCTATTA TGGTACGGAA CTTTTTTTAA TGAGGAATTT | 7620 |
| CATGATGATT TAGGAATTTT CTCTCTTGGA AAAGGCTTCC CCTGTGATGA AAATGATGTG | 7680 |
| CCAGCTAAAA TTGTGTGCCA TTTAAAAACT GAAAATATTT TAAAATTATT TGTCTATATT | 7740 |
| CTAAATTGAG CTTTGGATCA AACTTTAGGC CAGGACCAGC TCATGCGTTC TCATTCTTCC | 7800 |
| TTTTCTCACT CTTTCTCTCA TCACTCACCT CTGTATTCAT TCTGTTGTTT GGGATAGAAA | 7860 |
| AATCATAAAG AGCCAACCCA TCTCAGAACG TTGTGGATTG AGAGAGACAC TACATGACTC | 7920 |
| CAAGTATATG AGAAAAGGAC AGAGCTCTAA TTGATAACTC TGTAGTTCAA AAGGAAAAGA | 7980 |
| GTATGCCCAA TTCTCTCTAC ATGACATATT GAGATTTTTT TTAATCAACT TTTAAGATAG | 8040 |
| TGATGTTCTG TTCTAAACTG TTCTGTTTTA GTGAAGGTAG ATTTTTATAA AACAAGCATG | 8100 |
| GGGATTCTTT TCTAAGGTAA TATTAATGAG AAGGGAAAAA AGTATCTTTA ACAGCTCTTT | 8160 |
| GTTGAAGCCT GTGGTAGCAC ATTATGTTTA TAATTGCACA TGTGCACATA ATCTATTATG | 8220 |
| ATCCAATGCA AATACAGCTC CAAAAATATT AAATGTATAT ATATTTTAAA ATGCCTGAGG | 8280 |
| AAATACATTT TTCTTAATAA ACTGAAGAGT CTCAGTATGG CTATTAAAAT AATTATTAGC | 8340 |
| CTCCTTTGGT GTGGCTGCAA AACATCACAA AGTGACCGGT CTTGAGACCT GTGAACTGCT | 8400 |
| GCCCTGTTTA GTAAATAAAA TTAATGCATT TCTAGAGGGG GAATATCTGC CATCCAGTGG | 8460 |
| TGGAAATGTG GAGTAAAGAA GCTGGTGGTC TGCTTCTGTG CTGTATGCCA GCCTTTTGCC | 8520 |
| TTAAGTTGAG AGGAGGTCAA CTTTAGCTAC TGTCTTTGGT TTGAGAGCCA TGGCAAAAAA | 8580 |
| AAAAAAAGAA AAAAAGATCA AGTCGTCTTT GGTGAGCCAG TAAGGTGAAA GCTTGCTGAC | 8640 |
| TGTCCAAGGC ACAAGAGAAA ATTGAGGAAT TGAAATGCAA CCTGAGTATC AAACTAAATA | 8700 |
| TTCTAATCAA AGGTAGGTAC TGTTAGGTGG AATTCTATCA GCAGGCAACT GCAAATGAGA | 8760 |
| AGAAGATAGA AGGACGCCCG TCGGGACTTT GGAGGGCATT GTTATITTCC CAAAGAAAGA | 8820 |
| CGGCCAAGGG CAGAGGCATG GATTCTTTGC AGAGCACTTC CTTTTGGTTT TTCAGTACTG | 8880 |
| TTTCATAGAC AGTGGGCTCA CATGTTCCTG ATAGTGCTGC AGTTGCTTAG AAAGCATCCC | 8940 |
| AGTTAATTGC AGTAATTAGA ACTTCTGGAA TATGCTAGGG CAGAAGTATG TCAAGTATGT | 9000 |
| CACATGAAGA AAATGTGAAA TTCAAGAGTA ATCCACACGT GAGAAACTAG ACAATGTACA | 9060 |
| TTCATGTGTT CTCTTGAAAG GAAAGGGAGA GCTGTAAGCT TCACTCTGTC CTACACCGGA | 9120 |
| GAAAAGCAGG AATAACTTTA CCGTGGAAAT AATGTTTAGC TTTTATCAGA GAAAATTGTC | 9180 |
| CTTCTAGAGC ATAGAGTCCC AAAACTCAAT TCTGGTTTTC CCCTGTTTTT TTTTTTTTTT | 9240 |
| TTTTTCCCAA CATATGAACT GCAGCATATC ACTTTTTCTT TTTGTGCCTC AGGTTCCTCA | 9300 |
| CCTGTAAAAT TGAAAAATAT ATGTATTAAT AATATTATTA ATAATAATAA TGGTAATGTA | 9360 |
| GTACTTGTTT GTAAAGCACT TTGAGATCCT TGGTTGAAAG GCACCATAGG AGTGCCAAGT | 9420 |
| ATTATTATGT GGCCAAGGGG GTTATTTAAA CTGTCAGTTC CCAAAGGCCA GGAAAGGTTG | 9480 |
| GGGTCATTTT TCTTAAAGAC GAGCTGTAAA TATCAACTAG GCAGCCAATA GTGTTGACTA | 9540 |
| TGAAGATGCA AAACTATTAC TAGGCTGATA AAATCATAGT TTCTTAATGG CTACCAATAA | 9600 |
| GGCAAATATC ACAATAATAA ACGCCAAATT CCTTAGGGCG GACTATTTGA CAACCACATG | 9660 |
| GAAAACTTTG GGGGAGGCAT GAGGGGGGAA CATCTCAAAA TGCCAATGTA AAATTTAACT | 9720 |
| TACAGCAATA TTCACCAGCA GAAAATGTCT TTCATATGGA ATGATTTCAT GTTGCTAAGA | 9780 |
| AAAAGAATTC AA1TTGTAGT CCTGATTTGA ATACTAGAAT GTTGGCTATA ATAGTTCTGT | 9840 |
| TCTTACAACA CATGAAATTT TTTCGTTTTA TTTTATTTTG TTTTCATAGT GCATGTTCAT | 9900 |
| TTCTACTCAC AAACATGTTC TTGGTGTATT TCTTATGCAA ACAATCTTCA GGCAGCAAAG | 9960 |
| ATGTCTGTTA CATCTAAACT TGAATAATAA AGTTTTACCA CCAGTTACAC A | |

Seq ID NO: 68 Protein sequence:
Protein Accession #: NP_054831

| | |
|---|---|
| 1 11 21 31 41 51 | |
| \| \| \| \| \| \| | |
| MPYEVNAGYD FTNMVRKKNP PLRNVASEGE GQILEPIGTE SKVSGKNKEF SADQMSENTD | 60 |
| QSDAAELNHK EEHSLHVQDP SSSSKKDLKS AVLSEKAGFN YESPSKGGNF PSFPHDEVTD | 120 |
| RNMLAFSEPA AGGVCEPLKS PQRAEADDPQ DMACTPSGDS LETKEDQKMS PKATEETGQA | 180 |
| QSGQANCQGL SPVSVASKNP QVPSDGGVRL NKSKTDLLVN DNPDPAPLSP ELQDFKCNIC | 240 |
| GYGYYGNDPT DLIKHFRKYH LGLHNRTRQD AELDSKILAL HNMVQFSHSK DEQKVNRSVF | 300 |
| SGVLQDTNSS RPVLLNGTYD VQVTSGGTFI GIGRKTPDCQ GNTKYFRCKF CNFTYMGNSS | 360 |
| TELEQHFLQT HHNKIKASLP SSEVAKPSEK NSNKSIPALQ SSDSGDLGKW QDKITVKAGD | 420 |
| DTPVGYSVPI KPLDSSRQNG TEATSYYWCK FCSFSCESSS SLKLLEHYGG QHGAVQSGGL | 480 |
| NPELNDKLSR GSVINQNDLA KSSEGETMTK TDKSSSGAKK KDFSSKGAED NMVTSYNCQF | 540 |
| CDFRYSKSHG PDVIVVGPLL RHYQQLHNIH KCTKHCPFC PRGLCSPEKH LGEITYPFAC | 600 |
| RKSNCSHCAL LLLHLSPGAA GSSRVKHQCH QCSPTTPDVD VLLFHYESVH ESQASDVKQE | 660 |
| ANHLQGSDGQ QSVKESKEHS CTKCDFITQV EEEISRHYRR AHSCYKCRQC SFTAADTQSL | 720 |
| LEHFNTVHCQ EQGITTANGE EDGHAISTIK EEPKIDFRVY NLLTPDSKMG EPVSESVVKR | 780 |
| EKLEEKDGLK EKVWTESSSD DLRNVTWRGA DILRGSPSYT QASLGLLTPV SGTQEQTKTL | 840 |
| RDSPNVEAAH LARPIYGLAV ETKGFLQGAP AGGEKSGALP QQYPASGENK SKDESQSLLR | 900 |
| RRRGSGVFCA NCLTTKTSLW RKNANGGYVC NACGLYQKLH STPRPLNIIK QNNGEQIIRR | 960 |
| RTRKRLNPEA LQAEQLNXQQ RGSNEEQVNG SPLERRSEDH LTESHQREIP LPSLSKYEAQ | 1020 |
| GSLTKSHSAQ QPVLVSQThD IHKRMQPLHI QIKSPQESTG DPGNSSSVSE GKGSSERGSP | 1080 |
| IEKYMRPAKH PNYSPPGSPI EKYQYPLFGL PFVHNDFQSE ADWLRFWSKY KLSVPGNPHY | 1140 |
| LSHVPGLPNP CQNYVPYPTF NLPPHFSAVG SDNDIPLDLA LKTSRPGPTA NGASKEKTKA | 1200 |
| PPNVKNEGPL NVVKTEKVDR STQDELSTKC VHCGIVFLDE VMYALHMSCH GDSGPFQCSI | 1260 |
| CQHLCTDKYD FTTHIQRGLH RNNAQVEKNG KPKE | |

Seq ID NO: 69 DNA sequence
Nucleic Acid Accession #: XM_073879
Coding sequence: 1-387 (underlined sequences correspond to start and
stop codons)

| | |
|---|---|
| 1 11 21 31 41 51 | |
| \| \| \| \| \| \| | |
| ATGGGGTTTG GAGACCAGGG AACGGTGGAA GGGAGCCTAG GAACGTCGAA AAAACCACCT | 60 |
| GAAGTGAAAA TGTTTGGAGC CAGTCAAGGT TTGGTGACAA TGGAAACAAA CCAGTCCCTG | 120 |
| GCACAAGGCA CAGGCTGCTC AGTAGTAAAG GTAGACACTG TTCTCTTCGA GAGTTTATAC | 180 |
| CACTGCGGCT TTGAACACGG GAGCGTGATG CACTGCCTTG GGGATGATCA CCCCCAGGAA | 240 |
| GACAGGAAGG CTCACTTCTC TGCCCCAGTT GCAGCCATCG CCTCTCCAGC ACCGACTCCT | 300 |
| GTCTGTCCTG CACACCACTC AACACAGAGC ATCTGCCAGT TTCTACAGCA CTGCAGGCAG | 360 |
| AACACTCACT TGCAGGCTGC TAACTAA | |

Seq ID NO: 70 Protein sequence:
Protein Accession #: XP_073879

| | |
|---|---|
| 1 11 21 31 41 51 | |
| \| \| \| \| \| \| | |
| MGFGDQGTVE GSLGTSKKPP EVKMFGASQG LLTMETNQSL AQGTGCSVVK VDTVLFESLY | 60 |
| HCGFEHGSVM HCLGDDHPQE DRKAHFSAPV AAIASPAPTP VCPAHHSTQS ICQFLQHCRQ | 120 |
| NTHLQAAN | |

Seq ID NO: 71 DNA sequence
Nucleic Acid Accession #: AB033064
Coding sequence: 826-1986 (underlined sequences correspond to start
and stop codous)

| | |
|---|---|
| 1 11 21 31 41 51 | |
| \| \| \| \| \| \| | |
| GGGGAACAGC AAATTCAGTC ACAGACAATC CTCCACTCGG TCAAGAGCCA CTTTTCTCTT | 60 |
| CCTGCCTTGC CCCCCCGCAG GGGGTAAGGA ACTGAGCGTT TAATCTTTAG CCGGTTGGCT | 120 |
| ACCAGCTAAA ATTCTACTTA TCTTAGTTTC TAGTGGATAG CTTTCTTATT TTGCCCATGT | 180 |
| TTTCTTAGAA TCCCTGTTTA ATATACTTTT GTCAGTAGTA GTATCTAGGA GTAGCAGGGA | 240 |
| GAGTGACAAT AAATTAGCCC CTTCTTTTTT CCCTTGTCAT TCAGGCCCCT TTTCCTCTCC | 300 |
| AGAGGGAAAT TACCAGTAAA CTCTTCTAAA TCTTCCACCC CTTCTCAGTC ATACTGTGAA | 360 |
| GAAACACACT AAAGTGGACA TTATTTGACC AGTGAACACG AACCCAGCTT CAGGCATTGG | 420 |
| TTTGTTGTGG CACATGGAGA AACATCTCTT TTAAAATATC TCCCAATTAC CCTTTTCACA | 480 |
| ATTTGTATCC ACCTAGGATT TGCTGCTGGG GTAAGTCACT AGATTTATTT CTCAAAGCTC | 540 |
| CCCTCTCTAT GAGCTGAAAG ACTGACCAAC CATGAACACT AGTAGGGGAT GGGGAAAGGG | 600 |
| GACAGAGCAG AGCCAGTTGT TCCACACTTT GGGAAGCAGG AGTAGCTTTT ATCATCTTCC | 660 |
| TCTGGGGAGC AGGCATAGAG ACATAAACTG AGTGAAAATG GGTGGAGGAA GAACTTCTAT | 720 |
| ACCCACGAAC AACATGTGAA GAGAGAGAAC CAAACATAAA GTAAGGAGGG TAGACGTTAC | 780 |
| ATCCAAGAGG AAATAATCCA GGCAAGGAAG CACAAGCTGA TCAAGATGTG TAGTTCTGTG | 840 |
| GCTGCCAAGT TGTGGTTTTT GACAGATCGT CGCATCAGGG AAGACTATCC TCAAAAAGAG | 900 |
| ATTTTACGAG CATTGAAGGC CAAATGTTGT GAGGAGGAAC TGGACTTTAG GGCTGTGGTG | 960 |
| ATGGATGAGG TGGTGCTGAC AATCGAGCAA GGAAACCTGG GTCTGCGGAT CAATGGAGAG | 1020 |
| CTAATCACTG CCTACCCACA AGTGGTGGTA GTCAGAGTAC CAACCCCTTG GGTGCAAAGT | 1080 |
| GATAGTGACA TCACTGTTTT GCGCCATCTA GAGAAGATGG GATGTCGGTT AATGAACCGA | 1140 |
| CCTCAAGCCA TCCTGAACTG CGTTAATAAG TTCTGGACAT TTCAAGAGTT GGCTGGCCAT | 1200 |
| GGTGTTCCTC TGCCGGATAC TTTCTCTTAT GGTGGCCACG AAAATTTTGC TAAAATGATT | 1260 |
| GATGAGGCTG AAGTTCTGGA GTTCCCAATG GTAGTAAAGA ATACGCGGGG TCACAGAGGT | 1320 |
| AAAGCTGTTT TCTTGGCTCG AGATAAGCAC CATTTGGCTG ATCTAAGCCA TCTTATTCGC | 1380 |
| CATGAAGCGC CATACCTGTT CCAGAAGTAT GTTAAAGAGT CTCATGGACG GGATGTACGT | 1440 |
| GTCATTGTCG TGGGAGGCCG TGTGGTTGGC ACCATGTTAC GTTGTTCAAC AGATGGGAGA | 1500 |
| ATGCAAAGCA ACTGCTCATT AGGTGGTGTG GGGATGATGT GCTCATTGAG TGAACAAGGG | 1560 |
| AAGCAGCTAG CTATCCAGGT GTCTAATATC CTGGGGATGG ATGTGTGTGG CATTGACCTG | 1620 |
| CTGATGAAAG ATGACGGCTC CTTCTGCGTC TGTGAGGCCA ATGCAAATGT AGGTTTCATC | 1680 |
| GCCTTTGATA AGGCTTGTAA TCTAGATGTA GCTGGTATCA TAGCAGACTA TGCCGCCTCC | 1740 |
| CTTCTACCCT CTGGCCGGCT CACCCGGCGT ATGTCCGTGC TCTCCGTGGT GTCCACTGCC | 1800 |
| AGTGAGACTA GTGAGCCGGA GCTGGGTCCC CCAGCCAGCA CTGCTGTTGA CAACATGAGT | 1860 |
| GCAAGTTCCA GCTCTGTTGA CAGCGACCCT GAAAGCACGG AGCGAGAGCT GCTCACCAAG | 1920 |
| CTCCCAGGGG GCCTGTTCAA CATGAACCAG CTGGTAGCCA ATGAAATCAA ACTACTGGTG | 1980 |
| GACTGACTCC ACTGGTAATT AACCAACAAA ACCCTTGTAA AACTTTCTTT CTTCTTTTCT | 2040 |
| ATTTTTAAAA CCAACTTGCA ATGCTGTfCA TGGAGGATGC TCAGGAAGAT GAGAGAAAAT | 2100 |
| TAGTAGGATT AGTTGGAGAG AGTGGGAGAT AGATGAGACC TCTGCTAGTA AGATGTTACT | 2160 |
| TTCATTTACA AATCCTACAA ATAGAGAGGC AGAATAGGTG GGGTATAGAA AAATGTCAGG | 2220 |
| CTCTCATAGT TACCCTTTTA AATTGCTAAA AAATGTGTAT GCTCATAGGC CATGAGGAAC | 2280 |
| AAATACTTTT TTTTTTTCAT GGTCCCTGGC TTTTGTTTTT GTACAAAAAA AAATGGTTTT | 2340 |
| GCTACAAATA TCCAAGTAGC ATAACTTCAC ATTGTGTTGG AAGATTTGTC ATCAGTGAGG | 2400 |
| AAAACATCTG CTTAAATTAC AGGAATTTTT GTATTATACA GCTCTGAAAA TTCTGCCATT | 2460 |
| TCCTTATTAA CTAGCAGCTT TAGTTTGTAG TTTATGAAAT CTTGAGGGGC TCTTTTACTG | 2520 |
| GGATTTCTTA TTTTTTTGTT TTTTCCCGCT TAATTTGGTG GGAGGTCAAA TTGAATATAA | 2580 |
| CCCAATAAAG GCTTCTTAAT GACAAAATTG GCATGTTTGC ATGATGAAAT GGAAATGAAC | 2640 |
| AGTATTGCAA TGTCCGGTAT ACAAAATAAC ATTAATTCAA TGTAGATAAA ATTACACTAG | 2700 |
| TTTAAAATAT GTGCATTCAC TTGTATTTGT TAGTGTTTTA GTCTTTTTTG AAAGATGTGC | 2760 |
| TCTGTTAATG TTGCTTTTTT TTTTTTTTTT TAATACATGC TAGTCTAACA TTTCCTGCTC | 2820 |
| TATGCCTGCA TCTTTAACAA TGGCCAAAGT GAAGAAAATG CTACCTTTTT TGTTAACAAG | 2880 |
| ACACTGACTT GAAACATGTA CATTTAAAGC CTTfTATTTT TTCCCTTTTT GTTTTGGTAG | 2940 |
| TTGGGCATTT AAATAAGGAC AAGGAAAAAT ATTTTTGGGG GCAAATCAAG AGCCTATGAG | 3000 |
| TTCTAAGTAT AAAGCTGAAG TGATTTCGAA TGCCAGCGTT ATATATTTGC ATTTTTCACA | 3060 |
| TTTTACGAGG GAGTATATGT GTATGTGTGT GCACGCATGC ATGTGTATGT GTTTTGCTTT | 3120 |
| TTGTTTCCAT CAACTAATCA AAAAGGATAA TTTAGAAAAT GGAGCATGAT GGGAAACAGA | 3180 |
| GTTTTTGACT TTAAAAAACA GATGAGTTGT TTTCATAAGT AGACTCCACT GGGGTAGAGG | 3240 |
| TATTCACCTT AAAACATAGG GTGAGTAGAT GCTTTTTTAG GCCTTTTTGT GTATATGTAC | 3300 |
| GTTGTTTGTT TTTTTCCTTT TGTTTCTAGC CTGTTCAGTG TACAGTTTAT TCAAGGCTAC | 3360 |
| ATGCTTTTCT TTAATGCTTC TGGCTATGCA TTTTCTCTTT TTACATATAG GATTTGGGAT | 3420 |
| TGGGGGTGGG TTGGATGTTT TTGTTTGGGG ACTTATTTAG TAGTATTGAG TCTCTTATAG | 3480 |
| CCCTACTCTT AAGCCITCAA TACTGTCCAC TCTTTATATT CCTTTACTTG CAGAATTTAT | 3540 |
| AAAAGCCCCC AAACTGCATA TAATATGAGC CTTTAAAACA TGGGTAAAAC TAATCCCATT | 3600 |
| GATGGGTTTG GATGGTATGT TAAGAAATGG AGATGCTGCA GAGCCCAACG TAATTTTTTA | 3660 |
| AACAGCAAGT TTTCCATCTC CCTACGAATC CTCTGAAGCT TTTACCCAAG CCCTTTCTTG | 3720 |
| CCTCTCCAGT GCTATTTTCC TTCAGATGGA CCTTAAACAT AATTTCTTGG ACACTACTAG | 3780 |
| AGAGACTTCG AGGCAATAAT AAAAGATCAG TATTAACCAG CTATAACAGA GGTTTGATCA | 3840 |
| TGCITACTTG TACAGTFTTT CCCCCGTTTT AAAAAGGAAT GTAATAAAAT TTGTTTTTTC | 3900 |
| CATAGAATTA AATAATATTA AAATTGAGTG AAAGGTTGAT TGTTGATGAA TAGAATAGTA | 3960 |
| CCTCTCATCT GTGCAGTGTC TCATTTCACC TCAGAGAAAA GGATACATAA GAGGAGTTTG | 4020 |
| TAATTTATCT TAGGATATTC TAATTGCATT TAAAAGAACT TATCTTGCGC AGGGTAAATG | 4080 |
| GGGGACTCAC ATACATATAT TAATACCTCT GACTCATTAA CAGAAAGAAA TACTTGGTAC | 4140 |
| TfCTTTCGCT GAATGACCAT ACTGTGGAGG ATGCATACTA TTTGGTATAG AGAAATAAAT | 4200 |
| GAGGAAGAAA GAACTGCTTA ATTAAATfAT CATTCATATG TTCATATAGA GACCATCTGG | 4260 |
| TTGCCATGTG TATTATGACA CATACACTTT GAATAGTTAC ATATCACAAG TATGTAGTTC | 4320 |
| ATGTTTGTGT TGGTGGGGTA AGGCATCAGG AAAAATGTAG TTAGTCTTTT CTTAACTTAT | 4380 |
| ACCAAATTAA CCAACTATAT TATAGGAAAT ATGTGAAATT AGTTCATTAG CTTTATTCAC | 4440 |
| TATTATGCAT TCACATGATA TTAAAACGTA CACTCACATG TTAGAATGAA AAGAGCAGTA | 4500 |
| G3TATCTTAG ATTTTAAAAA CATGGATATC TTCTTGAATT CCTTCAAGAT TGAGGTAGAG | 4560 |
| AATAAGAGCA AATCATTCTG GAAGTACCTT AAGGAAACAA ACAGCAGCAG ATATTTAGGT | 4620 |
| TAAACTTATT TTCATAATTG TTTAATAACT TTTGTATAAT CTTCATTGCT ATTATGAGAG | 4680 |
| AGAATGTATA TATCAAATAT GTGTAATGAT AAAATCTGAA TTGTAAAATT TTTGTATATT | 4740 |
| GTTAAAATTG TAATTCTAAA TTGTATTTCA AAAATGATTA TTTCTGATAT TGTTTTTATG | 4800 |
| TCACCCATGA TGAAAACTGG ACTTTATATA TCTAAACATA CAAGTATGAA CTATTCTATT | 4860 |
| TAAAATTTTT AATAGTTTTT TTCTTTTTTG GTGCCTATAA TTGATTGGTC ATTTCTGCTG | 4920 |
| GCTTTTCTCC AATGAACATT GAAATCTTCC TGTATATGTT ACCAATAAGA AAACTACCCT | 4980 |
| GGAACAGTAG AAAAACCCAA CAAGAGACTT GGCATTCATC AAGCACATTA TCAGACTTTG | 5040 |
| AGAACATATT GAAGGCATTG ACTTTGAAAA TCATCTCTTT TTCTCAAGAA GAAAGCAATG | 5100 |
| GAGAAGCAAA TTTGTTTCAT TCAGTGAATC CCCAGTTTGG GGCTTGTGGG GCTTAGAGAC | 5160 |
| ATTGTGAAAT CAAATCTTGT GTTATACTTT TCTCCTGGCT CACTTTTTTT GAGAAGGTTT | 5220 |
| ATGGGCTATT TGGCTGGTGA GACACGATCC CCTCCTAAGA AAATGTAGGT GCTCAGACAG | 5280 |
| GTAACCACTG CTGCTACTGT TTTTATTTGT TTGTTTGTTC AATTTTATTT AAGATTTGTT | 5340 |
| TTTGTTGTAC TAGGATTTTA AAAAATGTAA TATATTGCAG GATTTATAAC CAG | |

Seq ID NO: 72 Protein sequence:
Protein Accession #: BAA86552

| | |
|---|---|
| 1 11 21 31 41 51 | |
| \| \| \| \| \| \| | |
| MCSSVAAKLW FLTDRRIRED YPQKEILRAL KAKCCEEELD FRAVVMDEVV LTIEQGNLGL | 60 |
| PJNGELITAY PQVVVVRVPT PWVQSDSDIT VLRHLEKMGC RLMNRPQAIL NCVNKFWTFQ | 120 |
| ELAGHGVPLP DTFSYGGHEN FAKMIDEAEV LEFPMVVKNT RGHRGKAVFL ARDKHHLADL | 180 |
| SHLIRHEAPY LFQKYVKESH GRDVRVIVVG GRVVGTMLRC STDGRMQSNC SLGGVGMMCS | 240 |
| LSEQGKQLAI QVSNILGMDV CGIDLLMKDD GSFCVCEANA NVGEIAFDKA CNLDVAGIIA | 300 |
| DYAASLLPSG RLTRRMSLLS VVSTASETSE PELGPPASTA VDNMSASSSS VDSDPESTER | 360 |
| ELLTKLPGGL FNMNQLLANE IKLLVD | |

Seq ID NO: 73 DNA sequence
Nucleic Acid Accession #: XM_040080.2
Coding sequence: 159-1104 (underlined sequences correspond to start
and stop condons)

| | |
|---|---|
| 1 11 21 31 41 51 | |
| \| \| \| \| \| \| | |
| CTGAGTGGGG GCGGGGACTG CTGGAGTTGC GGGGCCTGCC TGGGGTAGGG CGGGGCAGGA | 60 |
| CAGCTTGGAG ATAGGGCCCG GAATTGCGGG CGTCACTCTG CTCCTGCGAC CTAGCCAGGC | 120 |
| GTGAGGGAGT GACAGCAGCG CATTCGCGGG ACGAGAGCGA TGAGTGAGAA CGCCGCACCA | 180 |
| GGTCTGATCT CAGAGCTGAA GCTGGCTGTG CCCTGGGGCC ACATCGCAGC CAAAGCCTGG | 240 |
| GGCTCCCTGC AGGGCCCTCC AGTTCTCTGC CTGCACGGCT GGCTGGACAA TGCCAGCTCC | 300 |
| TTCGACAGAC TCATCCCTCT TCTCCCGCAA GACTTTTATT ACGTTGCCAT GGATTTCGGA | 360 |
| GGTCATGGGC TCTCGTCCCA TTACAGCCCA GGTGTCCCAT ATTACCTCCA GACTTTTGTG | 420 |
| AGTGAGATCC GAAGAGTTGT GGCAGCCTTG AAATGGAATC GATTCTCCAT TCTGGGCCAC | 480 |
| AGCTTCGGTG GCGTCGTGGG CGGAATGTTT TTCTGTACCT TCCCCGAGAT GGTGGATAAA | 540 |
| CTTATCTTGC TGGACACGCC GCTCTTTCTC CTGGAATCAG ATGAAATGGA GAACTTGCTG | 600 |
| ACCTACAAGC GGAGAGCCAT AGAGCACGTG CTGCAGGTAG AGGCCTCCCA GGAGCCCTCG | 660 |
| CACGTGTTCA GCCTGAAGCA GCTGCTGCAG AGGTTACTGA AGAGCAATAG CCACTTGAGT | 720 |
| GAGGAGTGCG GGGAGCTTCT CCTGCAAAGA GGAACCACGA AGGTGGCCAC AGGTCTGGTT | 780 |
| CTGAACAGAG ACCAGAGGCT CGCCTGGGCA GAGAACAGCA TTGAC1TCAT CAGGAGGGAG | 840 |
| CTGTGTGCGC ATfCCATCAG GAAGCTGCAG GCCCATGTCC TGTTGATCAA AGCAGTCCAC | 900 |
| GGATATTTTG ATTCAAGACA GAATTACTCT GAGAAGGAGT CCCTGTCGTT CATGATAGAC | 960 |
| ACGATGAAAT CCACCCTCAA AGAGCAGTTC CAGTTTGTGG AAGTCCCAGG CAATCACTGT | 1020 |
| GTCCACATGA GCGAACCCCA GCACGTGGCC AGTATCATCA GCTCCTTCTT ACAGTGCACA | 1080 |
| CACATGCTCC CAGCCCAGCT GTAGCTCTGG GCCTGGAACT ATGAAGACCT AGTGCTCCCA | 1140 |
| GACTCAACAC TGGGACTCTG AGTTCCTGAG CCCCACAACA AGGCCAGGGA TGGTGGGGAC | 1200 |
| AGGCCTCACT AGTCTTGAGG CCCAGCCTAG GATGGTAGTC AGGGGAAGGA GCGAGATTCC | 1260 |
| AACTTCAACA TCTGTGACCT CAAGGGGGAG ACAGAGTCTG GGTTCCAGGG CTGCTTTCTC | 1320 |
| CTGGCTAATA ATAAATATCC AGCCAGCTGG AGGAAGGAAG GGCAGGCTGG GCCCACCTAG | 1380 |
| CCTTTCCCTG CTGCCCAACT GGATGGAAAA TAAAAGGTTC TTGTATTCTC A | |

Seq ID NO: 74 Protein sequence:
Protein Accession #: XP_040080.1

| | |
|---|---|
| 1 11 21 31 41 51 | |
| \| \| \| \| \| \| | |
| MSENAAPGLI SELKLAVPWG HIAAKAWGSL QGPPVLCLHG WLDNASSFDR LIPLLPQDFY | 60 |
| YVAMDFGGHG LSSHYSPGVP YYLQTFVSEI RRVVAALKWN RFSILGHSFG GVVGGMFFCT | 120 |
| FPEMVDKLIL LDTPLFLLES DEMENLLTYK RRAIEHVLQV EASQEPSHVE SLKQLLQRLL | 180 |
| KSNSHLSEEC GELLLQRGTT KVATGLVLNR DQRLAWAENS IDFISRELCA HSIRKLQAHV | 240 |
| LLHZAVHGYF DSRQNYSEKE SLSFMIDTMK STLKEQFQFV EVPGNHCVHM SEPQBVASII | 300 |
| SSFLQCTHML PAQL | |

Seq ID NO: 75 DNA sequence
Nucleic Acid Accession #: NM_005794
Coding sequence: 434-1276 (underlined sequences correspond to start
and stop codons)

| | |
|---|---|
| 1 11 21 31 41 51 | |
| \| \| \| \| \| \| | |
| GGTTCCCTTC CACGCTGTGA AGCTTTGTTC TTTTGGTCTT CATGATAAAT CTTGCTGCTG | 60 |
| CTCACTCGTT GGGTCCGTGC CACCTTTAAG AGCTGTAACA CTCACCGCGA AGGTCTGCAA | 120 |
| CTICACTCCT GGGGCCAGCA AGACCACGAA TGCACCGAGA GGAATGAACA ACTCTGGACA | 180 |
| CACCATCTTT AAGAACCGTA ATACTCACCG CAAGGGTCTG CAACTTCATT CTTGAAGTCA | 240 |
| GTGAGGCCAA GAACCCATCA ATTCCGTACA CATTTTGGTG ACTTTGAAGA GACTGTCACC | 300 |
| TATCACCAAG TGGTGAGACT ATTGCCAAGC AGTGAGACTA TTGCCAAGTG GTGAGACCAT | 360 |
| CACCAAGCGG TGAGACTATC ACCTATCGCC AAGTGGCCTG ATTCAGCAGG AAGCATCTCA | 420 |
| GACACCAACC ACTATGCTGT CAGCAGTTGC CCGGGGCTAC CAGGGCTGGT TTCATCCCTG | 480 |
| TGCTAGGCTT TCTGTGAGGA TGAGCAGCAC CGGGATAGAC AGGAAGGGCG TCCTGGCTAA | 540 |
| CCGGGTAGCC GTGGTCACGG GGTCCACCAG TGGGATCGGC TTTGCCATCG CCCGACGTCT | 600 |
| GGCCCGGGAC GGGGCCCACG TGGTCATCAG CAGCCGGAAG CAGCAGAACG TGGACCGGGC | 660 |
| CATGGCCAAG CTGCAGGGGG AGGGGCTGAG TGTGGCGGGC ATTGTGTGCC ACGTGGGGAA | 720 |
| GGCTGAGGAC CGGGAGCAGC TGGTGGCCAA GGCCCTGGAG CACTGTGGGG GCGTCGACTT | 780 |
| CCTGGTGTGC AGCGCAGGGG TCAACCCTCT GGTAGGGAGC ACTCTGGGGA CCAGTGAGCA | 840 |
| GATCTGGGAC AAGATCCTAA GTGTGAACGT GAAGTCCCCA GCCCTGCTGC TGAGCCAGTT | 900 |
| GCTGCCCTAC ATGGAGAACA GGAGGGGTGC TGTCATCCTG GTCTCTTCCA TTGCAGCTTA | 960 |
| TAATCCAGTA GTGGCGCTGG GTGTCTACAA TGTCAGCAAG ACAGCGCTGC TGGGTCTCAC | 1020 |
| TAGAACACTG GCATTGGAGC TGGCCCCCAA GGACATCCGG GTAAACTGCG TGGTTCCAGG | 1080 |
| AATTATAAAA ACTGACTTCA GCAAAGTGTT TCATGGGAAT GAGTCTCTCT GGAAGAACTT | 1140 |
| CAAGGAACAT CATCAGCTGC AGAGGATTGG GGAGTCAGAG GACTGTGCAG GAATCGTGTC | 1200 |
| CTTCCTGTGC TCTCCAGATG CCAGCTACGT CAACGGGGAG AACATTGCGG TGGCAGGCTA | 1260 |
| CTCCACTCGG CTCTGAGAGG AGTGGGGGCG GCTGCGTAGC TGTGGTCCCA GCCCAGGAGC | 1320 |
| CTGAGGGGGT GTCTAGGTGA TCATTTGGAT CTGGAGCAGA GTCTGCCATT CTGCCAGACT | 1380 |
| AGCAATTfGG GGGCTTACTC ATGCTAGGCT TGAGGAAGAA GAAAAACGCT TCGGCATTCT | 1440 |
| CC | |

Seq ID NO: 76 Protein sequence:
Protein Accession #: NP_005785

| | |
|---|---|
| 1 11 21 31 41 51 | |
| \| \| \| \| \| \| | |
| MLSAVARGYQ GWFHPCARLS VRMSSTGIDR KGVLANRVAV VTGSTSGIGF AIARRLARDG | 60 |
| AHVVISSRKQ QNVDRAMAKL QGEGLSVAGI VCHVGKAEDR EQLVAKALEH CGGVDFLVCS | 120 |
| AGVNPLVGST LGTSEQIWDK ILSVNVKSPA LLLSQLLPYM ENRRGAVILV SSIAAYNPVV | 180 |
| ALGVYNVSKT ALLGLTRTLA LELAPKDIRV NCVVPGIIKT DFSKVFHGNE SLWKNFKEHH | 240 |
| QLQRIGESED CAGIVSFLCS PDASYVNGEN IAVAGYSTRL | |

Seq ID NO: 77 DNA sequence
Nucleic Acid Accession #: NM_002425
Coding sequence: 26-1453 (underlined sequences correspond to start and
stop codons)

| | |
|---|---|
| 1 11 21 31 41 51 | |
| \| \| \| \| \| \| | |
| AAAGAAGGTA AGGGCAGTGA GAATGATGCA TCTTGCATTC CTTGTGCTGT TGTGTCTGCC | 60 |
| AGTCTGCTCT GCCTATCCTC TGATTGGGGC AGCAAAAGAG GAGGACTCCA ACAAGGATCT | 120 |
| TGCCCAGCAA TACCTAGAAA AGTACTACAA CCTCGAAAAG GATGTGAAAC AGTTTAGAAG | 180 |
| AAAGGACAGT AATCTCATTG TTAAAAAAAT CCAAGGAATG CAGAAGTTCC TTGGGTTGGA | 240 |
| GGTGACAGGG AAGCTAGACA CTGACACTCT GGAGGTGATG CGCAAGCCCA GGTGTGGAGT | 300 |
| TCCTGACGTT GGTCACTTCA GCTCCTTTCC TGGCATGCCG AAGTGGAGGA AAACCCACCT | 360 |
| TACATACAGG ATTGTGAATT ATACACCAGA TTTGCCAAGA GATGCTGTTG ATTCTGCCAT | 420 |
| TGAGAAAGCT CTGAAAGTCT GGGAAGAGGT GACTCCACTC ACAYrCTCCA GGCTGTATGA | 480 |
| AGGAGAGGCT GATATAATGA TCTCTTTCGC AGTTAAAGAA CATGGAGACT TTTACTCTTT | 540 |
| TGATGGCCCA GGACACAGTT TGGCTCATGC CTACCCACCT GGACCTGGGC TTTATGGAGA | 600 |
| TATTCACTTT GATGATGATG AAAAATGGAC AGAAGATGCA TCAGGCACCA ATTTATTCCT | 660 |
| CGTTGCTGCT CATGAACTTG GCCACTCCCT GGGGCTCTTT CACTCAGCCA ACACTGAAGC | 720 |
| TTTGATGTAC CCACTCTACA ACTCATTCAC AGAGCTCGCC CAGTTCCGCC TTTCGCAAGA | 780 |
| TGATGTGAAT GGCATTCAGT CTCTCTACGG ACCTCCCCCT GCCTCTACTG AGGAACCCCT | 840 |
| GGTGCCCACA AAATCTGTTC CTTCGGGATC TGAGATGCCA GCCAAGTGTG ATCCTGCTTT | 900 |
| GTCCTTCGAT GCCATCAGCA CTCTGAGGGG AGAATATCTG TTCTYfAAAG ACAGATATTT | 960 |
| TTGGCGAAGA TCCCACTGGA ACCCTGAACC TGAATTTCAT TTGATTTCTG CATTTTGGCC | 1020 |
| CTCTC1TCCA TCATAT1TGG ATGCTGCATA TGAAGTTAAC AGGAGGGACA CCGTTTTTAT | 1080 |
| TTTTAAAGGA AATGAGTTCT GGGCCATCAG AGGAAATGAG GTACAAGCAG GTTATCCAAG | 1140 |
| AGGCATCCAT ACCCTGGGTT TTCCTCCAAC CATAAGGAAA ATTGATGCAG CTGTTTCTGA | 1200 |
| CAAGGAAAAG AAGAAAACAT ACTTCTTTGC AGCGGACAAA TACTGGAGAT TTGATGAAAA | 1260 |
| TAGCCAGTCC ATGGAGCAAG GCTTCCCTAG ACTAATAGCT GATGACTTTC CAGGAGTTGA | 1320 |
| GCCTAAGG1T GATGCTGTAT TACAGGCATT TGGATTTTTC TACTTCTTCA GTGGATCATC | 1380 |
| ACAGTTTGAG TTTGACCCCA ATGCCAGGAT GGTGACACAC ATATTAAAGA GTAACAGCTG | 1440 |
| GTTACATTGC TAGGCGAGAT AGGGGGAAGA CAGATATGGG TGTTTTTAAT AAATCTAATA | 1500 |
| ATTATTCATC TAATGTATTA TGAGCCAAAA TGGTTAATTT TTCCTGCATG TTCTGTGACT | 1560 |
| GAAGAAGATG AGCCTTGCAG ATATCTGCAT GTGTCATGAA GAATGTTTCT GGAATTCTTC | 1620 |
| ACTTGCTTTT GAATTGCACT GAACAGAATT AAGAAATACT CATGTGCAAT AGGTGAGAGA | 1680 |
| ATGTATTTTC ATAGATGTGT TATTACTTCC TCAATAAAAA GTTTTATTTT GGGCCTGTTC | 1740 |
| CTT | |

Seq ID NO: 78 Protein sequence:
Protein Accession #: NP_002416

| | |
|---|---|
| 1 11 21 31 41 51 | |
| \| \| \| \| \| \| | |
| MHLAFLVLLC LPVCSAYPLS GAAKEEDSNK DLAQQYLEKY YNLEKDVKQF RRXDSNLIVK | 60 |
| KIQGMQKFLG LEVTGKLDTD TLEVMRICPRC GVPDVGHFSS FPGMPKWRKT HLTYRVNYT | 120 |
| PDLPRDAVDS AIEKALKVWE EVTPLTFSRL YEGEADIMIS FAVKEHGDFY SFDGPGHSLA | 180 |
| HAYPPGPGLY GDIHFDDDEK WTEDASGTNL FLVAAHELGH SLGLFHSANT EALMYPLYNS | 240 |
| FTELAQFRLS QDDVNGIQSL YGPPPASTEE PLVPTKSVPS GSEMFAKCDP ALSFDAISTL | 300 |
| RGEYLFFKDR YFWRRSHWNP EPEEHLISAF WPSLPSYLDA AYEVNSRDTV FIFKGNEFWA | 360 |
| IRGNEVQAGY PRGIHTLGFP PTIRKIDAAV SDKEKKKTYF FAADKYWRFD ENSQSMEQGF | 420 |
| PRLIADDFPG VEPKVDAVLQ AFGFFYFFSG SSQFEFDPNA RMVTHILKSN SWLHC | |

Seq ID NO: 79 DNA sequence
Nucleic Acid Accession #: NM_000493.1
Coding sequence: 1-2043 (underlined sequences correspond to start and
stop codons)

| | |
|---|---|
| 1 11 21 31 41 51 | |
| \| \| \| \| \| \| | |
| ATGCTGCCAC AAATACCCTT TTTGCTGCTA GTATCCTTGA ACTTGGTTCA TGGAGTGTTT | 60 |
| TACGCTGAAC GATACCAAAC GCCCACAGGC ATAAAAGGCC CACTACCCAA CACCAAGACA | 120 |
| CAGTTCTTCA TTCCCTACAC CATAAAGAGT AAAGGTATAG CAGTAAGAGG AGAGCAAGGT | 180 |
| ACTCCTGGTC CACCAGGCCC TGCTGGACCT CGAGGGCACC CAGGTCCTTC TGGACCACCA | 240 |
| GGAAAACCAG GCTACGGAAG TCCTGGACTC CAAGGAGAGC CAGGGTTGCC AGGACCAGCG | 300 |
| GGACCATCAG CTGTAGGGAA ACCAGGTGTG CCAGGACTCC CAGGAAAACC AGGAGAGAGA | 360 |
| GGACCATATG GACCAAAAGG AGATGTTGGA CCAGCTGGCC TACCAGGACC CCGGGGCCCA | 420 |
| CCAGGACCAC CTGGAATCCC TGGACCGGCT GGAATTTCTG TGCCAGGAAA ACCTGGACAA | 480 |
| CAGGGACCCA CAGGAGCCCC AGGACCCAGG GGCTTTCCTG GAGAAAAGGG TGCACCAGGA | 540 |
| GTCCCTGGTA TGAATGGACA GAAAGGGGAA ATGGGATATG GTGCTCCTGG TCGTCCAGGT | 600 |
| GAGAGGGGTC TTCCAGGCCC TCAGGGTCCC ACAGGACCAT CTGGCCCTCC TGGAGTGGGA | 660 |
| AAAAGAGGTG AAAATGGGGT TCCAGGACAG CCAGGCATCA AAGGTGATAG AGGTTTTCCG | 720 |
| GGAGAAATGG GACCAATTGG CCCACCAGGT CCCCAAGGCC CTCCTGGGGA ACGAGGGCCA | 780 |
| GAAGGCATTG GAAAGCCAGG AGCTGCTGGA GCCCCAGGCC AGCCAGGGAT TCCAGGAACA | 840 |
| AAAGGTCTCC CTGGGGCTCC AGGAATAGCT GGGCCCCCAG GGCCTCCTGG CTTTGGGAAA | 900 |
| CCAGGCTTGC CAGGCCTGAA GGGAGAAAGA GGACCTGCTG GCCTTCCTGG GGGTCCAGGT | 960 |
| GCCAAAGGGG AACAAGGGCC AGCAGGTCTT CCTGGGAAGC CAGGTCTGAC TGGACCCCCT | 1020 |
| GGGAATATGG GACCCCAAGG ACCAAAAGGC ATCCCGGGTA GCCATGGTCT CCCAGGCCCT | 1080 |
| AAAGGTGAGA CAGGGCCAGC TGGGCCTGCA GGATACCCTG GGGCTAAGGG TGAAAGGGGT | 1140 |
| TCCCCTGGGT CAGATGGAAA ACCAGGGTAC CCAGGAAAAC CAGGTCTCGA TGGTCCTAAG | 1200 |
| GGTAACCCAG GGTTACCAGG TCCAAAAGGT GATCCTGGAG TTGGAGGACC TCCTGGTCTC | 1260 |
| CCAGGCCCTG TGGGCCCAGC AGGAGCAAAG GGAATGCCCG GACACAATGG AGAGGCTGGC | 1320 |
| CCAAGAGGTG CCCCTGGAAT ACCAGGTACT AGAGGCCCTA TTGGGCCACC AGGCATTCCA | 1380 |
| GGATTCCCTG GGTCTAAAGG GGATCCAGGA AGTCCCGGTC CTCCTGGCCC AGCTGGCATA | 1440 |
| GCAACTAAGG GCCTCAATGG ACCCACCGGG CCACCAGGGC CTCCAGGTCC AAGAGGCCCC | 1500 |
| TCTGGAGAGC CTGGTCTTCC AGGGCCCCCT GGGCCTCCAG GCCCACCAGG TCAAGCAGTC | 1560 |
| ATGCCTGAGG GTTTTATAAA GGCAGGCCAA AGGCCCAGTC TTTCTGGGAC CCCTCTTGTT | 1620 |
| AGTGCCAACC AGGGGGTAAC AGGAATGCCT GTGTCTGCTT TTACTGTTAT TCTCTCCAAA | 1680 |
| GCTTACCCAG CAATAGGAAC TCCCATACCA TTTGATAAAA TTTTGTATAA CAGGCAACAG | 1740 |
| CATTATGACC CAAGGACTGG AATCTTTACT TGTCAGATAC CAGGAATATA CTATTTTTCA | 1800 |
| TACCACGTGC ATGTGAAAGG GACTCATGTT TGGGTAGGCC TGTATAAGAA TGGCACCCCT | 1860 |
| GTAATGTACA CCTATGATGA ATACACCAAA GGCTACCTGG ATCAGGCTTC AGGGAGTGCC | 1920 |
| ATCATCGATC TCACAGAAAA TGACCAGGTG TGGCTCCAGC TTCCCAATGC CGAGTCAAAT | 1980 |
| GGCCTATACT CCTCTGAGTA TGTCCACTCC TCTTTCTCAG GATTCCTAGT GGCTCCAATG | 2040 |
| TGAGTACACC CCACAGAGCT AATCTAAATC TTGTGCTAGA AAAAGCATTC TCTAACTCTA | 2100 |
| CCCCACCCTA CAAAATGCAT ATGGAGGTAG GCTGAAAAGA ATGTAATTTT TATTTTCTGA | 2160 |
| AATACAGATT TGAGCTATCA GACCAACAAA CCTTCCCCCT GAAAAGTGAG CAGCAACGTA | 2220 |
| AAAACGTATG TGAAGCCTCT CTTGAATTTC TAGTTAGCAA TCTTAAGGCT CTTTAAGGTT | 2280 |
| TTCTCCAATA TTAAAAAATA TCACCAAAGA AGTCCTGCTA TGTTAAAAAC AAACAACAAA | 2340 |
| AAACAAAGCA ACAAAAAAAA AAATTAAAAA AAAAAACAGA AATAGAGCTC TAAGTTATGT | 2400 |
| GAAATTTGAT TTGAGAAACT CGGCATTTCC TTTTTAAAAA AGCCTGTTTC TAACTATGAA | 2460 |
| TATGAGAACT TCTAGGAAAC ATCCAGGAGG TATCATATAA CTTTGTAGAA CTTAAATACT | 2520 |
| TGAATATTCA AATTTAAAAG ACACTGTATC CCCTAAAATA TTTCTGATGG TGCACTACTC | 2580 |
| TGAGGCCTGT ATGGCCCCTT TCATCAATAT CTATTCAAAT ATACAGGTGC ATATATACTT | 2640 |
| GTTAAAGCTC TTATATAAAA AAGCCCCAAA ATATTGAAGT TCATCTGAAA TGCAAGGTGC | 2700 |
| TTTCATCAAT GAACCTTTTC AAAACTTTTC TATGATTGCA GAGAAGCTTT TTATATACCC | 2760 |
| AGCATAACTT GGAAACAGGT ATCTGACCTA TTCTTATTTA GTTAACACAA GTGTGATTAA | 2820 |
| TTTGATTTCT TTAATTCCTT ATTGAATCTT ATGTGATATG ATTTTCTGGA TTTACAGAAC | 2880 |
| ATTAGCACAT GTACCTTGTG CCTCCCATTC AAGTGAAGTT ATAATTTACA CTGAGGGTTT | 2940 |
| CAAAATTCGA CTAGAAGTGG AGATATATTA TTTATTTATG CACTGTACTG TATTTTTATA | 3000 |
| TTGCTGTTTA AAACTTTTAA GCTGTGCCTC ACTTATTAAA GCACAAAATG TTTTACCTAC | 3060 |
| TCCTTATTTA CGACACAATA AAATAACATC AATAGATTTT TAGGCTGAAT TAATTTGAAA | 3120 |
| GCAGCAATTT GCTGTTCTCA ACCATTCTTT CAAGGCTTTT CATTCGACAC AATAAAATAA | 3180 |
| CATCAATAGA TTTTTAGG | |

Seq ID NO: 80 Protein sequence:
Protein Accession #: NP_000484.1

| | |
|---|---|
| 1 11 21 31 41 51 | |
| \| \| \| \| \| \| | |
| MLPQIPELLL VSLNLVHGVF YAERYQTPTG IKGPLPNTKT QFFIPYTIKS KGIAVRGEQG | 60 |
| TPGPPGPAGP RGHPGPSGPP GKPGYGSPGL QGEPGLPGPP GPSAVGKPGV PGLPGKPGER | 120 |
| GPYGPKGDVG PAGLPGPRGP PGPPGIPGPA GISVPGKPGQ QGPTGAPGPR GFPGEKGAPG | 180 |
| VPGMNGQKGE MGYGAPGRPG ERGLPGPQGP TGPSGPPGVG KRGENGVPGQ PGIKGDRGFP | 240 |
| GEMGPIGPPG PQGPPGERGP EGIGKPGAAG APGQPGIPGT KGLPGAPGIA GPPGPPGFGK | 300 |
| PGLPGLKGER GPAGLPGGPG AKGEQGPAGL PGKPGLTGPP GNMGPQGPKG IPGSHGLPGP | 360 |
| KGETGPAGPA GYPGAKGERG SPGSDGKPGY PGKPGLDGPK GNPGLPGPKG DPGVGGPPGL | 420 |
| PGPVGPAGAK GMPGHNGEAG PRGAPGIPGT RGPIGPPGIP GFPGSKGDPG SPGPPGPAGI | 480 |
| ATKGLNGPTG PPGPPGPRGP SGEPGLPGPP GPPGPPGQAV MPEGFIKAGQ RPSLSGTPLV | 540 |
| SANQGVTGMP VSAFTVILSK AYPAIGTPIP FDK1LYNRQQ HYDPRTGIFT CQIPGIYYFS | 600 |
| YHVHVKGTHV WVGLYKNGTP VMYTYDEYTK GYLDQASGSA IIGLTENDQV WLQLPNAESN | 660 |
| GLYSSEYVHS SFSGFLVAPM | |

Seq ID NO: 81 DNA sequence
Nucleic Acid Accession #: NM_000786
Coding sequence: 332-1861 (underlined sequences correspond to start
and stop codons)

| | |
|---|---|
| 1 11 21 31 41 51 | |
| \| \| \| \| \| \| | |
| CGCGATTCTC AGGGATTGAT CCGCCTCTTC AGGTAAGTTA TCTTCCGGCC CCGTACCACT | 60 |
| GTGCCACAGG CGCAGCCCGC TTCCTCAGGT GCCCTATCCC GCGCAGAAGA CCACGGCTTC | 120 |
| ACAGAGTGTT ATTTAAGGGC GTGGCCAGCG GAACATCCCG CCCCATTCTG TGACGCACGG | 180 |
| GGTGGCGCGC GTGGGACCCG AGGGGTGGGG CTGGGTTTAG TAGGAGACCT GGGGCAAGGC | 240 |
| CCCCTGTGGA CGACCATCTG CCAGCTTCTC TCGTTCCGTC GATTGGGAGG AGCGGTGGCG | 300 |
| ACCTCGGCCT TCAGTGTTTC CGACGGAGTG AATGGCGGCG GCGGCTGGGA TGCTGCTGCT | 360 |
| GGGCTTGCTG CAGGCGGGTG GGTCGGTGCT GGGCCAGGCG ATGGAGAAGG TGACAGGCGG | 420 |
| CAACCTCTTG TCCATGCTGC TGATCGCCTG CGCCTTCACC CTCAGCCTGG TCTACCTGAT | 480 |
| CCGTCTGGCC GCCGGCCACC TGGTCCAGCT GCCCGCAGGG GTGAAAAGTC CTCCATACAT | 540 |
| TTTCTCCCCA ATTCCATTCC TTGGGCATGC CATAGCATTT GGGAAAAGTC CAATTGAATT | 600 |
| TCTAGAAAAT GCATATGAGA AGTATGGACC TGTATTTAGT TTTACCATGG TAGGCAAGAC | 660 |
| ATTTACTTAC CTTCTGGGGA GTGATGCTGC TGCACTGCTT TTTAATAGTA AAAATGAAGA | 720 |
| CCTGAATGCA GAAGATGTCT ACAGTCGCCT GACAACACCT GTGTTTGGGA AGGGAGTTGC | 780 |
| ATACGATGTG CCTAATCCAG TTTTCTTGGA GCAGAAGAAA ATGTTAAAAA GTGGCCTTAA | 840 |
| CATAGCCCAC TTTTAACAGC ATGTTTTTAT AATTGAAAAA GAAACAAAGG AATACTTTGA | 900 |
| GAGTTGGGGA GAAAGTGGAG AAAAAAATGT GTTTGAAGCT CTTTCTGAGC TCATAATITT | 960 |
| AACAGCTAGC CATTGTTTGC ATGGAAAGGA AATCAGAAGT CAACTCAATG AAAAGGTAGC | 1020 |
| ACAGCTGTAT GCAGATTTGG ATGGAGGTTT CAGCCATGCA GCCTGGCTCT TACCAGGTTG | 1080 |
| GCTGCCTTTG CCTAGTTTCA GACGCAGGGA CAGAGCTCAT CGGGAAATCA AGGATATTTT | 1140 |
| CTATAAGGCA ATCCAGAAAC GCAGACAGTC TCAAGAAAAA ATTGATGACA TTCTCCAAAC | 1200 |
| TTTACTAGAT GCTACATACA AGGATGGGCG TCCTTTGACT GATGATGAAG TAGGAGGGAT | 1260 |
| GCTTATTGGA TTACTCTTGG CAGGGCAGCA TACATCCTCA ACTACTAGTG CTTGGATGGG | 1320 |
| CTTCTTTTTG GCCAGAGACA AAACACTTCA AAAAAAATGT TATTTAGAAC AGAAAACAGT | 1380 |
| CTGTGGAGAG AATCTGCCTC CTCfTAACTTA TGACCAGCTC AAGGATCTAA ATTTACTGA | 1440 |
| TCGCTGTATA AAAGAAACAT TAAGACTTAG ACCTCCTATA ATGATCATGA TGAGAATGGC | 1500 |
| CAGAACTCCT CAGACTGTGG CAGGGTATAC CATTCCTCCA GGACATCAGG TGTGTGTTTC | 1560 |
| TCCCACTGTC AATCAAAGAC TTAAAGACTC ATGGGTAGAA CGCCTGGACT TTAATCCTGA | 1620 |
| TCGCTACTTA CAGGATAACC CAGCATCAGG GGAAAAGTTT GCCTATGTGC CAGGTGGAGC | 1680 |
| TGGGCGTCAT CGTTGTATTG GGGAAAATTT TGCCTATGGT CAAATTAAGA CAATTTGGTC | 1740 |
| CACTATGCTT CGTTTATATG AATTTGATCT CATTGATGGA TACTTTCCCA CTGTGAATTA | 1800 |
| TACAACTATG ATTCACACCC CTGAGAACCC AGTTATCCGT TACAAACGAA GATCAAAATG | 1860 |
| AAAAAGGTTG CAAGGAACGA ATATATGTGA TTATCACTGT AAGCCACAAA GGCATTCGAA | 1920 |
| GAGAATGAAG TGTACAAAAC AACTCTTGTA GTTTACTGTT TTTTTTAGTG TGTAATTCTA | 1980 |
| AAAGCCAGTT TATGATTTAG GATTTTGTTA ACTGAATGGT TCTATCAAAT ATAATAGCAT | 2040 |
| TTGACACATT TTCTAATAGT TATGATACTT ATACATGTGC TTTCAGGAAG TTCCTTGGTG | 2100 |
| AAACAATTGT TGAGGGGGGA TCTAGGTAAT TGGCAGATTC TAAATAATAT AATTTCCAGA | 2160 |
| TAGTAATTTT AAGAGTACTC ATCGCTCTTG CCAAATAAGT TCAGGGTATT CAAATCTTGG | 2220 |
| ACTAGTCCTG CAAGGTATAA AGAATAAAAA TCCCAGTGAG ATACTTGGAA ACCACAGTTT | 2280 |
| ATTATTATTT ATCTGGGCAA TTATTGTGTG TGTGAGGATG GAAGGGTAGG GAATAATCGA | 2340 |
| ACATCTAAAG CCTTGAATAA GAGAATACTA ATTGTTTTGG TATGATGATA CTCAGAAATG | 2400 |
| GAGATATTAT AGGAAAAAGA AATCCTTTGG AATTTTAACT AAAATCACTG CATATGGGAA | 2460 |
| ATTAAGAGAT CCAGGACCAT ATTTGATAAG AGTTCCTAAA AATAATGTAA TTATTAATGC | 2520 |
| TAAAGACTGC TCATGTATCT TGATCTAATT ACTAAATAAA TTACATATTT ATTTACCTGA | 2580 |
| TAAATATGTA TCTAGTTCTA CAAGGTCACA TTTATGTGGA AGTCCAAAGT CAAGTCCTTA | 2640 |
| GGGGATAATT TTGTTTTGGG CTCAGTTGTT CCCTGCTTCC TTTTTTTTTT TTTTTTTTTT | 2700 |
| TTGAGATGGA GTCTCGCTCT GTTGCCCAGG CTGGAGTGCA GTGGTGCGAT CTCAGCTCAC | 2760 |
| TGCATCCTCT GCCTCCCGGG TTCAAGCAAT TCTCTGCCTC AGCCTCCCAA GTAGTTGGGA | 2820 |
| TTACAGGCAC CTGCCACCAT GCCTGGCTAA TTTTTTGTAT TTTTAGTAGA GACGGGGGTT | 2880 |
| TCACTATGTT GGCTAGGCTG GTCTTGAACT CCTGAGCCTC GTGAGTCCAC CCGCCTTGGC | 2940 |
| CTCCCAAAGT GCTGGGATTA CAGGCATGAG CCACCGCACC TGGCCTTCCC TGCTTCCTCT | 3000 |
| CTAGAATCCA ATTAGGGATG TTTGTTACTA CTCATATTGA TTAAAACAGT TAACAAACTT | 3060 |
| TTITCTTTTT AAAATGTGAG ATCAGTGAAC TCTGGTTTTA AGATAATCTG AAACAAGGTC | 3120 |
| CTTGGGAGTA ATAAAATTGG TCACATTCTG TAAAGCACAT TCTGTTTAGG AATCAACTTA | 3180 |
| TCTCAAATTG TAACTCGGGG CCTAACTATA TGAGATGGCT GAAAAAATAC CACATCGTCT | 3240 |
| GTTTTCACTA GGTGATGCCA AAATATTTTG CTTTATGTAT ATTACAGTTC TTTTTAAAAC | 3300 |
| ACTGGAAGAC TCATGTTAAA CTCTAATTGT GAAGGCAGAA TCTCTGCTAA TTTTTCAGAT | 3360 |
| TAAAATTCTC TTTGAAAAAA T | |

Seq ID NO: 82 Protein sequence
Protein Accession #: NP_000777

| | |
|---|---|
| 1 11 21 31 41 51 | |
| \| \| \| \| \| \| | |
| MAAAAGMLLL GLLQAGGSVL GQAMEKVTGG NLLSMLLIAC AITLSLVYLI RLAAGHLVQL | 60 |
| PAGVKSPPYI FSPIPFLGHA IAFGKSPIEF LENAYEKYGP VESFTMVGKT FTYLLGSDAA | 120 |
| ALLFNSKNED LNAEDVYSRL TTPVFGKGVA YDVPNPVFLE QKKMLKSGLN IAHFKQBVSI | 180 |
| IEKETKEYFE SWGESGEKNV FEALSELIIL TASHCLHGKE IRSQLNEKVA QLYADLDGGF | 240 |
| SHAAWLLPGW LPLPSFRRRD RAHREIKDIF YKAIQKRRQS QEKIDDILQT LLDATYKDGR | 300 |
| PLTDDEVAGM LIGLLLAGQH TSSTTSAWMG FFLARDKTLQ KKCYLEQKTV CGENLPPLTY | 360 |
| DQLKDLNLLD RCIKETLRLR PPIMIMMRMA RTPQTVAGYT IPPGHQVCVS PTVNQRLKDS | 420 |
| WVERLDFNPD RYLQDNPASG EKFAYVPFGA GRHRCIGENF AYVQIKTIWS TMLRLYEFDL | 480 |
| IDGYFPTVNY TTMIHTPENP VIRYKRRSK | |

Seq ID NO: 83 DNA sequence
Nucleic Acid Accession #: NM_006551.2
Coding sequence: 64-336 (underlined sequences correspond to start and
stop coduns)

| | |
|---|---|
| 1 11 21 31 41 51 | |
| \| \| \| \| \| \| | |
| AATTCTAGAA GTCCAAATCA CTCATTGTTT GTGAAAGCTG AGCTCACAGC AAAACAAGCC | 60 |
| ACCATGAAGC TGTCGGTGTG TCTCCTGCTG GTCACGCTGG CCCTCTGCTG CTACCAGGCC | 120 |
| AATGCCGAGT TCTGCCCAGC TCTTGTTTCT GAGCTGTTAG ACTTCTTCTT CATTAGTGAA | 180 |
| CCTCTGTTCA AGTTAAGTCT TGCCAAATTT GATGCCCCTC CGGAAGCTGT TGCAGCCAAG | 240 |
| TTAGGAGTGA AGAGATGCAC GGATCAGATG TCCCTTCAGA AACGAAGCCT CATTGCGGAA | 300 |
| GTCCTGGTGA AAATATTGAA GAAATGTAGT GTGTGACATG TAAAAACTTT CATCCTGGTT | 360 |
| TCCACTGTCT TTCAATGACA CCCTGATCTT CACTGCAGAA TGTAAAGGTT TCAACGTCTT | 420 |
| GCTTTAATAA ATCACTTGCT CTAC | |

Seq ID NO: 84 Protein sequence:
Protein Accession #: NP_006542.1

| | |
|---|---|
| 1 11 21 31 41 51 | |
| \| \| \| \| \| \| | |
| MKLSVCLLLV TLALCCYQAN AEFCPALVSE LLDFFFISEP LFKLSLAKFD APPEAVAAKL | 60 |
| GVKRCTDQMS LQKRSLIAEV LVKILKKCSV | |

Seq ID NO: 85 DNA sequence
Nucleic Acid Accession #: NM_002317.1
Coding sequence: 231-1484 (underlined sequences correspond to start
and stop condons)

| | |
|---|---|
| 1 11 21 31 41 51 | |
| \| \| \| \| \| \| | |
| GGGCCAGGAC TGAGAAAGGG GAAAGGGAAG GGTGCCACGT CCGAGCAGCC GCCTTGACTG | 60 |
| GGGAAGGGTC TGAATCCCAC CCTTGGCATT GCTTGGTGGA GACTGAGATA CCCGTGCTCC | 120 |
| GCTCGCCTCC TTGGTTGAAG ATTTCTCCTT CCCTCACGTG ATTTGAGCCC CGTTTTTATT | 180 |
| TTCTGTGAGC CACGTCCTCC TCGAGCGGGG TCAATCTGGC AAAAGGAGTG ATGCGCTICG | 240 |
| CCTGGACCGT GCTCCTGCTC GGGCCTTTGC AGCTCTGCGC GCTACTGCAC TGCGCCCCTC | 300 |
| CCGCCGCCGG CCAACAGCAG CCCCCGCGCG AGCCGCCGGC GGCTCCGGGC GCCTGGCGCC | 360 |
| AGCAGATCCA ATGGGAGAAC AACGGGCAGG TGTTCAGCTT GCTGAGCCTG GGCTCACAGT | 420 |
| ACCAGCCTCA GCGCCGCCGG GACCCGGGCG CCGCCGTCCC TGGTGCAGCC AACGCCTCCG | 480 |
| CCCAGCAGCC CCGCACTCCG ATCCTGCTGA TCCGCGACAA CCGCACCGCC GCGGGGCGAA | 540 |
| CGCGGACGGC CGGCTCATCT GGAGTCACCG CTGGCCGCCC CAGGCCCACC GCCCGTCACT | 600 |
| GGTTCCAAGC TGGCTACTCG ACATCTAGAG CCCGCGAAGC TGGGCCCTCG CGCGCGGAGA | 660 |
| ACCAGACAGC GCCGGGAGAA GTTCCTGCTC TCAGTAACCT GCGGCCGCCC AGCCGCGTGG | 720 |
| ACGGCATGGT GGGGGACGAC CCTTACAACC CCTACAAGTA CTCTGACGAC AACCCTTATT | 780 |
| ACAACTACTA CGATACTTAT GAAAGGCCCA GACCTGGGGG CAGGTACCGG CCCGGATACG | 840 |
| GCACTGGCTA CTTCCAGTAC GGTCTCCCAG ACCTGGTGGC CGACCCCTAC TACATCCAGG | 900 |
| CGTCCACGTA CGTGCAGAAG ATGTCCATGT ACAACCTGAG ATGCGCGGCG GAGGAAAACT | 960 |
| GTCTGGCCAG TACAGCATAC AGGGCAGATG TCAGAGATTA TGATCACAGG GTGCTGCTCA | 1020 |
| GATTICCCCA AAGAGTGAAA AACCAAGGGA CATCAGATTT CTTACCCAGC CGACCAAGAT | 1080 |
| ATTCCTGGGA ATGGCACAGT TGTCATCAAC ATTACCACAG TATGGATGAG TTTAGCCACT | 1140 |
| TGTACCTGCT TGATGCCAAC ACCCAGAGGA GATGGGCTGA AGGCCACAAA GCAAGTTTCT | 1200 |
| GTCTTGAAGA CACATCCTGT GACTATGGCT ACCACAGGCG ATTTGCATGT ACTGCACACA | 1260 |
| CACAGGGATT GAGTCCTGGC TGTTATGATA CCTATGGTGC AGACATAGAC TGCCAGTGGA | 1320 |
| TTGATATTAC AGATGTAAAA CCTGGAAACT ATATCCTAAA GGTCAGTGTA AACCCCAGCT | 1380 |
| ACCTGGTTCC TGAATCTGAC TATACCAACA ATGTTGTGCG CTGTGACATT CGCTACACAG | 1440 |
| GACATCATGC GTATGCCTCA GGCTGCACAA TTTCACCGTA TTAGAAGGCA AAGCAAAACT | 1500 |
| CCCAATGGAT AAATCAGTGC CTGGTGTTCT GAAGTGGGAA AAAATAGACT AACTTCAGTA | 1560 |
| GGATTTATGT ATTTTGAAAA AGAGAACAGA AAACAACAAA AGAATTTTTG TTTGGACTGT | 1620 |
| TTTCAATAAC AAAGCACATA ACTGGATTTT GAACGCTTAA GTCATCATTA CTTGGGAAAT | 1680 |
| TTTTAATGTT TATTATTTAC ATCACTTTGT GAATTAACAC AGTGTTTCAA TTCTGTAATT | 1740 |
| ACATATTTGA CTCTTTCAAA AAAAAAAAAA AAAAAAAAAA | |

Seq ID NO: 86 Protein sequence:
Protein Accession #: NP_002308.1

| | |
|---|---|
| 1 11 21 31 41 51 | |
| \| \| \| \| \| \| | |
| MRFAWTVLLL GPLQLCALVH CAPPAAGQQQ PPREPPAAPG AWRQQIQWEN NGQVFSLLSL | 60 |
| GSQYQPQRRR DPGAAVPGAA NASAQQPRTP LLLIRDNRTA AGRTRTAGSS GVTAGRPRPT | 120 |
| ARSWEQAGYS TSRAREAGPS RAENQTAPGE VPALSNLRPP SRVDGMVGDD PYNPYKYSDD | 180 |
| NPYYNYYDTY ERPRPGGRYR PGYGTGYEQY GLPDLVADPY YIQASTYVQK MSMThLRCAA | 240 |
| EENCLASTAY IADVRDYDHR VLLRFPQRVK NQGTSDFLPS RPRYSWEWHS CHQHYHSMDE | 300 |
| FSHLYLLDAN TQRRWAEGHK ASFCLEDTSC DYGYHRRFAC TAHTQGLSPG CYDTYGADID | 360 |
| CQWIDITDVK PGNYILKVSV NPSYLVPESD YTNNVVRCDI RYTGHHAYAS GCTISPY | |

Seq ID NO: 87 DNA sequence
Nucleic Acid Accession #: NM 006419.1
Coding sequence: 91-420 (underlined sequences correspond to start and
stop codons)

| | |
|---|---|
| 1 11 21 31 41 51 | |
| \| \| \| \| \| \| | |
| TTCGGCACTT GGGAGAAGAT GTTTGAAAAA ACTGACTCTG CTAATGAGCC TGGACTCAGA | 60 |
| GCTCAAGTCT GAACTCTACC TCCAGACAGA ATGAAGTTCA TCTCGACATC TCTGCTTCTC | 120 |
| ATGCTtCTGG TCAGCAGCCT CTCTCCAGTC CAAGGTGTTC TGGAGGTCTA TTACACAAGC | 180 |
| TTGAGGTGTA GATGTGTCCA AGAGAGCTCA GTCTTTATCC CTAGACGCTT CATTGATCGA | 240 |
| ATTCAAATCT TGCCCCGTGG GAATGGTTGT CCAAGAAAAG AAATCATAGT CTGGAAGAAG | 300 |
| AACAAGTCAA TtGTGTGTGT GGACCCTCAA GCTGAATGGA TACAAAGAAT GATGGAAGTA | 360 |
| TTGAGAAAAA GAAGTTCTTC AACTCTACCA GTTCCAGTGT TTAAGAGAAA GATTCCCTGA | 420 |
| TGCTGATATT TCCACTAAGA ACACCTGCAT TCTTCCCTTA TCCCTGCTCT GGATTTTAGT | 480 |
| TTTGTGCTTA GTTAAATCTT TTCCAGGGAG AAAGAACTTC CCCATACAAA TAAGGCATGA | 540 |
| GGACTATGTG AAAAATAACC TTGCAGGAGC TGATGGGGCA AACTCAAGCT TCTTCACTCA | 600 |
| CAGCACCCTA TATACACTTG GAGTTTTCAT TCTIATTCAT CAGGGAGGAA AGTTTCTTTG | 660 |
| AAAATAGTTA TTCAGTTATA AGTAATACAG GATTATTTTG ATTATATACT TGTTGTTTAA | 720 |
| TGTTTAAAAT TTCTTAGAAA ACAATGGAAT GAGAATTTAA GCCTCAAATT TGAACATGTG | 780 |
| GCTTGAATTA AGAAGAAAAT TATGGCATAT ATTAAAAGCA GGCTTCTATG AAAGACTCAA | 840 |
| AAAGCTGCCT GGGAGGCAGA TGGAACTTGA GCCTGTCAAG AGGCAAAGGA ATCCATGTAG | 900 |
| TAGATATCCT CTGCTTAAAA AGTCACTACG GAGGAGAATT AAGTCCTACT TTTAAAGAAT | 960 |
| TTCTTTATAA AATTTACTGT CTAAGAITAA TAGCATTCGA AGATCCCCAG ACTTCATAGA | 1020 |
| ATACTCAGGG AAAGCATTTA AAGGGTGATG TACACATGTA TCCTTTCACA CA1TTGCCTT | 1080 |
| GACAAACTTC TTTCACTCAC ATCTTTTTCA CTGACTTTTT TTGTGGGGGC GGGGCCGGGG | 1140 |
| GGACTCTGGT ATCTAATTCT TTAATGATTC CTATAAATCT AATGACATTC AATAAAGTTG | 1200 |
| AGCAAACATT TTACTT | |

Seq ID NO: 88 Protein sequence:
Protein Accession #: NP_006410.1

| | |
|---|---|
| 1 11 21 31 41 51 | |
| \| \| \| \| \| \| | |
| MCFISTSLLL MLLVSSLSPV QGVLEVYYTS LRCRCVQESS VFIPRRFIDR IQILPRGNGC | 60 |
| PRKEIIVWKX NKSIVCVDPQ AEWIQRMMEV LRKRSSSTLP VPVFKRKIP | |

Seq ID NO: 89 DNA sequence
Nucleic Acid Accession #: NM_002652
Coding sequence: 37-477 (underlined sequences correspond to start and
stop codons)

| | |
|---|---|
| 1 11 21 31 41 51 | |
| \| \| \| \| \| \| | |
| CTTCTCTGGG ACACATTGCC TTCTGTTTTC TCCAGCATGC GCTTGCTCCA GCTCCTGTTC | 60 |
| AGGGCCAGCC CTGCCACCCT GCTCCTGGTT CTCTGCCTGC AGTTGGGGGC CAACAAAGCT | 120 |
| CAGGACAACA CTCGGAAGAT CATAATAAAG AATTTTGACA TTCCCAAGTC AGTACGTCCA | 180 |
| AATGACGAAG TCACTGCAGT GCTTGCAGTT CAAACAGAAT TGAAAGAATG CATGGTGGTT | 240 |
| AAAACTTACC TCATTAGCAG CATCCCTCTA CAAGGTGCAT TTAACTATAA GTATACTGCC | 300 |
| TGCCTATGTG ACGACAATCC AAAAACCTTC TACTGGGACT TTTACACCAA CAGAACTGTG | 360 |
| CAAATTGCAG CCGTCGTTGA TGTTAITCGG GAATTAGGCA TCTGCCCTGA TGATGCTGCT | 420 |
| GTAATCCCCA TCAAAAACAA CCGGTTTTAT ACTATTGAAA TCCTAAAGGT AGAATAATGG | 480 |
| AAGCCCTGTC TGTTTGCCAC ACCCAGGTGA TTTCCTCTAA AGAAACTTGG CTGGAATTTC | 540 |
| TGCTGTGGTC TATAAAATAA ACTTCTTAAC ATGCTT | |

Seq ID NO: 90 Protein sequence:
Protein Accession #: NP_002643.1

| | |
|---|---|
| 1 11 21 31 41 51 | |
| \| \| \| \| \| \| | |
| MRLLQLLFRA SPATLLLVLC LQLGANKAQD NTRKIIIKNF DIPKSVRPND EVTAVLAVQT | 60 |
| ELKECMVVKT YLISSIPLQG AENYKYTACL CDDNPKTFYW DFYTNRTVQI AAVVDVIRBL | 120 |
| GICPDDAAVI PIKNNRFYTI EILKV | |

Seq ID NO: 91 DNA sequence
Nucleic Acid Accession #: AK00034.1
Coding sequence: 85-975 (underlined sequences correspond to start and
stop codons)

| | |
|---|---|
| 1 11 21 31 41 51 | |
| \| \| \| \| \| \| | |
| GATAGCGCCG GGCAGAGGGA CCCGGCTACC CTGGACAGCG CATCGCCGCC CGCCCGGGTC | 60 |
| GCCGCGCCAC AGCCGCTGCG GATCATGGAA CATCTAAAGG CCTTTGATGA TGAAATCAAT | 120 |
| GCTTTTTTGG ACAATATGTT TGGACCGCGA GATTCTCGAG TCAGAGGGTG GTTCACGTTG | 180 |
| GACTCTTACC TTCCTACCTT TTTTCTTACT GTCATGTATC TGCTCTCAAT ATGGCTGGGT | 240 |
| AACAAGTATA TGAAGAACAG ACCTGCTCTT TCTCTCAGGG GTATCCTCAC CTTGTATAAT | 300 |
| CTTGGAATCA CACTTCTCTC CGCGTACATG CTGGCAGAGC TCATTCTCTC CACTTGGGAA | 360 |
| GGAGGCTACA ACTTACAGTG TCAAGATCTT ACCAGCTCAG GGGAAGCTGA CATCCGGGTA | 420 |
| GCCAAGGTGC TTTGGTGGTA CTATTTCTCC AAATCAGTAG AGTTCCTGGA CACAATTTTC | 480 |
| TTCGTTTTGC GGAAAAAAAC GAGTCAGATT ACTTTTCTTC ATGTATATCA TCATGCTTCT | 540 |
| ATGTTTAACA TCTGGTGGTG TGTCTTGAAC TGGATACCTT GTGGACAAAG TTTCTTTGGA | 600 |
| CCAACACTGA ACAGTTTTGT CCACATTCTT ATGTACTCCT ACTATGGACT TTCTGTGTTT | 660 |
| CCATCTATGC ACAAGTATCT TTGGTGGAAG AAATATCTCA CACAGGCTCA GCTGGTGCAG | 720 |
| TTCGTGCTCA CCATCACGCA CACCATGAGC GCCGTCGTGA AACCGTGTGG CTTCCCCTTC | 780 |
| GGTTGTCTCA TCTTCCAGTC ATCTTATATG CTAACGTTAG TCATCCTCTT CTTAAATTTT | 840 |
| TATGTTCAGA CATACCGAAA AAAGCCAATG AAGAAAGATA TGCAAGAGCC ACCTGCAGGG | 900 |
| AAAGAAGTGA AGAATGGTTT TTCCAAAGCC TACTTCACTG CAGCAAATGG AGTGATGAAC | 960 |
| AAGAAAGCAC AATAAAAATG AGTAACAGAA AAAGCACATA TACTAGCCTA ACAGATTGGC | 1020 |
| TTGTTTTAAA GCAAAGACTG AATTGAAGGT TACATGTTTT AGGATAAACT AATTTCTTTT | 1080 |
| GAGTTCATAA ATCAflTGTA CCCAGAATGT ATTAATATAT TGCTATTAGG TTAATCTGTT | 1140 |
| AACTGAATGC TTTGATCAGC ATTGAGGTGA TGCTCACCTC CGAGGACCTC AGAACTGGTG | 1200 |
| CAGCTTCTCT CTCCCTCCCT CCCACAGACT GAACCITTCG CCAGAAGCTG TCCTTATAAC | 1260 |
| GCCTTATACG CATACACAGC CAGGAAACGT GGAGCATTGT TTCTCACAGA GAGTCTCCAA | 1320 |
| ATAAAAAGGG TTTTGTTCAG ATTAAAATGT TTACAACAAA ATGTTAATTA TATTCTAAAT | 1380 |
| ACAGGGTATG TTCTAATCTA TATTAAGCAA TAATGCCAGT GCATAATCAT TCCATTfGTT | 1440 |
| CCTTTAGCAA TCAACCCCAG AAAATATTAA AATGGGATCA TACACAGAAG ATAGAAAAAT | 1500 |
| CTAGCAAAAC TTCTCTTTCT GTAAGCCAGA GTCTTGTCTA TCAGATTCCC ACAACCACTC | 1560 |
| CTGATTCTAA ATTTAGTGAT ATGGTAATGA AATTGGTA3T TATTTTAAAT ATTAGTTATT | 1620 |
| CTAAGGAGAA AAAAATGCTT CTGCAAGATT TTCATAATTC AGGGGCTGTG GATAGGATTG | 1680 |
| TTCCTCTGTT TCCCTAATCA TTCATCTGTT CATGTCTCCC TCTTGTGCCA GTCAGCCTAG | 1740 |
| GTTATACAGA TGCCATGCTC CACACCACGA GCAGTGTACA AATCTGGCTG CCCGTTTACT | 1800 |
| TTCTGAGCAA GCACTGGAGT CCACTCCGAC CTTTTTCTTT GAACATGCAT GCTGCTGGAA | 1860 |
| TATGTATAAA TCAGAACTAG CAGAAGTAGC AGAGTGATGG GAGCAAAATA GGCACTGAAT | 1920 |
| TCGTCAACTC TTTTTTGTGA GCCTACTTGT GAATATTACC TCAGATACCT GTTGTCACTC | 1980 |
| TTCACAGGTT ATTTAAGTTC TTGAAGCTGQ GAGGAAAAAG ATGGAGTAGC TTGGAAAGAT | 2040 |
| TCCAGCACTG AGCCGTGAGC CGGTCATGAG CCACGATAAA AAATGCCAGT TTGGCAAACT | 2100 |
| CAGCACTCCT GTTCCCTGCT CAGGTATATG CGATCTCTAC TGAGAAGCAA GCACAAAAGT | 2160 |
| AGACCAAAGT ATTAATGAGT ATTTCCTTTC TCCATAAGTG CAGGACTGTT ACTCACTACT | 2220 |
| AAACTCTACC AAGAATGGAA ACCAAGAATA TTTTCTGAAG ATTTTTTTGA AGATTAATTT | 2280 |
| ATACCCTATA AAATAAAACT TGTTAGCTTC GATGAAGTCA AAAAAAAAAA AAAAAAAAAA | |

Seq ID NO: 92 Protein sequence:
Protein Accession #: BAA91096.1

| | |
|---|---|
| 1 11 21 31 41 51 | |
| \| \| \| \| \| \| | |
| MEHLKAFDDE INAFLDNMFG PRTSRVRGWF TLDSYLPTFF LTVMYLLSIW LGNKYMKNRP | 60 |
| ALSLRGILTL YNLGITLLSA YMLAELILST WEGGYNLQCQ DLTSAGEADI RVAKVLWWYY | 120 |
| FSKSVEFLDT IEFVLRKKTS QITFLHVYHH ASMFNIWWCV LNWIPCGQSF FGPTLNSFVH | 180 |
| ILMYSYYGLS VFPSMHKYLW WKKYLTQAQL VQFVLTITHT MSAVVKPCGF PFGCLIFQSS | 240 |
| YMLTLVILFL NFYVQTYRJCX PMKKDMQEPP AGKEVICNGFS KAYFTAANGV MNKKAQ | |

Seq ID NO: 93 DNA sequence
Nucleic Acid Accession #: NM_000044
Coding sequence: 1115-3874 (underlined sequences correspond to start
and stop codons)

| | |
|---|---|
| 1 11 21 31 41 51 | |
| \| \| \| \| \| \| | |
| CGAGATCCCG GGGAGCCAGC TTGCTGGGAG AGCGGGACGG TCCGGAGCAA GCCCACAGGC | 60 |
| AGAGGAGGCG ACAGAGGGAA AAAGGGCCGA GCTAGCCGCT CCAGTGCTGT ACAGGAGCCG | 120 |
| AAGGGACGCA CCACGCCAGC CCCAGCCCGG CTCCAGCGAC AGCCAACGCC TCTTGCAGCG | 180 |
| CGGCGGCTTC GAAGCCGCCG CCCGGAGCTG CCCTTTCCTC TTCGGTGAAG TTTTTAAAAG | 240 |
| CTGCTAAATA CTCGGAGGAA GCAAGGAAAG TGCCTGGTAG GACTGACGGC TGCCTTTGTC | 300 |
| CTCCTCCTCT CCACCCCGCC TCCCCCCACC CTGCCTTCCC CCCCTCCCCC GTCTTCTCTC | 360 |
| CCGCAGCTGC CTCAGTCGGC TACTCTCAGC CAACCCCCCT CACCACCCTT CTCCCCACCC | 420 |
| GCCCCCCCGC CCCCGTCGGC CCAGCGCTGC CAGCCCGAGT TTGCAGAGAG GTAACTCCCT | 480 |
| TTGGCTGCGA GCGGGCGAGC TAGCTGCACA TTGCAAAGAA GGCTCTTAGG AGCCAGGCGA | 540 |
| CTGGGGAGCG GCTTCAGCAC TGCAGCCACG ACCCCCCTGG TTAGAA3TCC GGCGGAGAGA | 600 |
| ACCCTCTGTT TTCCCCCACT CTCTCTCCAC CTCCTCCTGC CTTCCCCACC CCGAGTGCGG | 660 |
| AGCAGAGATC AAAAGATGAA AAGGCAGTCA GGTCTTCAGT AGCCAAAAAA CAAAACAAAC | 720 |
| AAAAACAAAA AAGCCGAAAT AAAAGAAAAA GATAATAACT CAGTTCTTAT TTGCACCTAC | 780 |
| TTCAGTGGAC ACTGAATTTG GAAGGTGGAG GATTTTGTTT TTTTCTTTTA AGATCTGGGC | 840 |
| ATCTTTTGAA TCTACCCTTC AAGTATTAAG AGACAGACTG TGAGCCTAGC AGGGCAGATC | 900 |
| TTGTCCACCG TGTGTCTTCT TCTGCACGAG ACTTTGAGGC TGTCAGAGCG CTTTTTGCGT | 960 |
| GGTTGCTCCC GCAAGTTTCC TTCTCTGGAG CTTCCCGCAG GTGGGCAGCT AGCTGCAGCG | 1020 |
| ACTACCGCAT CATCACAGCC TGTTGAACTC TTCTGAGCAA GAGAAGGGGA GGCGGGGTAA | 1080 |
| GGGAAGTAGG TGGAAGATTC AGCCAAGCTC AAGGATGGAA GTGCAGTTAG GGCTGGGAAG | 1140 |
| GGTCTACCCT CGGCCGCCGT CCAAGACCTA CCGAGGAGCT TTCCAGAATC TGTTCCAGAG | 1200 |
| CGTGCGCGAA GTGATCCAGA ACCCGGGCCC CAGGCACCCA GAGGCCGCGA GCGCAGCACC | 1260 |
| TCCCGGCGCC AGTTTGCTGC TGCTGCAGCA GCAGCAGCAG CAGCAGCAGC AGCAGCAGCA | 1320 |
| GCAGCAGCAG CAGCAGCAGC AGCAGCAAGA GACTAGCCCC AGGCAGCAGC AGCAGCAGCA | 1380 |
| GGGTGAGGAT GGTTCTCCCC AAGCCCATCG TAGAGGCCCC ACAGGCTACC TGGTCCTGGA | 1440 |
| TGAGGAACAG CAACCTTCAC AGCCGCAGTC GGCCCTGGAG TGCCACCCCG AGAGAGGTTG | 1500 |
| CGTCCCAGAG CCTGGAGCCG CCGTGGCCGC CAGCAAGGGG CTGCCGCAGC AGCTGCCAGC | 1560 |
| ACCTCCGGAC GAGGATGACT CAGCTGCCCC ATCCACGTTG TCCCTGCTGG GCCCCACTTT | 1620 |
| CCCCGGCTTA AGCAGCTGCT CCGCTGACCT TAAAGACATC CTGAGCGAGG CCAGCACCAT | 1680 |
| GCAACTCCTT CAGCAACAGC AGCAGGAAGC AGTATCCGAA GGCAGCAGCA GCGGGAGAGC | 1740 |
| GAGGGAGGCC TCGGGGGCTC CCACTTCCTC CAAGGACAAT TACTTAGGGG GCACTTCGAC | 1800 |
| CATTTCTGAC AACGCCAAGG AGTTGTGTAA GGCAGTGTCG GTGTCCATGG GCCTGGGTGT | 1860 |
| GGAGGCGTTG GAGCATCTGA GTCCAGGGGA ACAGCTTCGG GGGGATTGCA TGTACGCCCC | 1920 |
| ACTTTTGGGA GTTCCACCCG CTGTGCGTCC CACTCCTTGT GCCCCATTGG CCGAATGCAA | 1980 |
| AGGTTCTCTG CTAGACGACA GCGCAGGCAA GAGCACTGAA GATACTGCTG AGTATTCCCC | 2040 |
| TTTCAAGGGA GGTTACACCA AAGGGCTAGA AGGCGAGAGC CTAGGCTGCT CTGGCAGCGC | 2100 |
| TGCAGCAGGG AGCTCCGGGA CACTTGAACT GCCGTCTACC CTGTCTCTCT ACAAGTCCGG | 2160 |
| AGCACTGGAC GAGGCAGCTG CGTACCAGAG TCGCGAGTAG TACAACTTTC CACTGGCTCT | 2220 |
| GGCCGGACCG CCGCCCCCTC CGCCGCCTCC CCATCCCCAC GCTCGCATCA AGCTGGAGAA | 2280 |
| CCCGCTGGAC TACGGCAGCG CCTGGGCGGC TGCGGCGGCG CAGTGCGGCT ATGGGGACGT | 2340 |
| GGCGAGCCTG CATGGCGCGG GTGCAGCGGG ACCCGGTTCT GGGTCACCCT CAGCCGCCGC | 2400 |
| TTCCTCATCC TGGCACACTC TCTTCACAGC CGAAGAAGGC CAGTTGTATG GACCGTGTGG | 2460 |
| TGGTGGTGGG GGTGGTGGCG GCGGCGGCGG CGGCGGCGGC GGCGGCGGCG GCGGCGGCGG | 2520 |
| CGGCGGCGGC GAGGCGGGAG CTGTAGCCCC CTACGGCTAC ACTCGGCCCC CTCAGGGGCT | 2580 |
| GGCGGGCCAG GAAAGCGACT TCACCGCACC TGATGTGTGG TACCCTGGCG GCATGGTGAG | 2640 |
| CAGAGTGCCC TATCCCAGTC CCACTTGTGT CAAAAGCGAA ATGGGCCCCT GGATGGATAG | 2700 |
| CTACTCCGGA CCTTACGGGG ACATGCGTTT GGAGACTGCC AGGGACCATG TTTTGCCCAT | 2760 |
| TGACTATTAC TTTCCACCCC AGAAGACCTG CCTGATCTGT GGAGATGAAG CTTCTGGGTG | 2820 |
| TCACTATGGA GCTCTCACAT GTGGAAGCTG CAAGGTCTTC TTCAAAAGAG CCGCTGAAGG | 2880 |
| GAAACAGAAG TACCTGTGCG CCAGCAGAAA TGATTGCACT ATTGATAAAT TCCGAAGGAA | 2940 |
| AAATTGTCCA TCTTGTCGTC TTCGGAAATG TTATGAAGCA GGGATGACTC TGGGAGCCCG | 3000 |
| GAAGCTGAAG AAACTTGGTA ATCTGAAACT ACAGGAGGAA GGAGAGGCTT CCAGCACCAC | 3060 |
| CAGCCCCACT GAGGAGACAA CCCAGAAGCT GACAGTGTCA CACATTGAAG GCTATGAATG | 3120 |
| TCAGCCCATC TTTCTGAATG TCCTGGAAGC CATTGAGCCA GGTGTAGTGT GTGCTGGACA | 3180 |
| CGACAACAAC CAGCCCGACT CCTTTGCAGC CTTGCTCTCT AGCCTCAATG AACTGGGAGA | 3240 |
| GAGACAGCTT GTACACGTGG TCAAGTGGGC CAAGGCCITG CCTGGCTTCC GCAACTTACA | 3300 |
| CGTGGACGAC CAGATGGCTG TCATTCAGTA CTCCTGGATG GGGCTCATGG TGTTTGCCAT | 3360 |
| GGGCTGGCGA TCCTTCACCA ATGTCAACTC CAGGATGCTC TACTTCGCCC CTGATCTGGT | 3420 |
| TTTCAATGAG TACCGCATGC ACAAGTCCCG GATGTACAGC CAGTGTGTCC GAATGAGGCA | 3480 |
| CCTCTCTCAA GAGTTTGGAT GGCTCCAAAT CACCCCCCAG GAATTCCTGT GCATGAAAGC | 3540 |
| ACTGCTACTC TTCAGCATTA TTCCAGTGGA TGGGCTGAAA AATCAAAAAT TCTTTGATGA | 3600 |
| ACTTCGAATG AACTACATCA AGGAACTCGA TCGTATCATT GCATGCAAAA GAAAAAATCC | 3660 |
| CACATCCTGC TCAAGACGCT TCTACCAGCT CACCAAGCTC CTGGACTCCG TGCAGCCTAT | 3720 |
| TGCGAGAGAG CTGCATCAGT TCACTTTTGA CCTGCTAATC AAGTCACACA TGGTGAGCGT | 3780 |
| GGACTTTCCG GAAATGATGG CAGAGAICAT CTCTGTGCAA GTGCCCAAGA TCCTTTCTGG | 3840 |
| GAAAGTCAAG CCCATCTATT TCCACACCCA GTGAAGCATT GGAAACCCTA TTTCCCCACC | 3900 |
| CCAGCTCATG CCCCCTTTCA GATGTCTTCT GCCTGTTATA ACTCTGCACT ACTCCTCTGC | 3960 |
| AGTGCCTTGG GGAATTTCCT CTATTGATGT ACAGTCTGTC ATGAACATGT TCCTGAATTC | 4020 |
| TATTTGCTGG GCTTTTTTTT TTCTTTCTTC TCCTTTCTTT TTCTTCTTCC CTCCCTATCT | 4080 |
| AACCCTCCCA TGGCACCTTC AGACTTTGCT TCCCATTGTG GCTCCTATCT GTGTTTTGAA | 4140 |
| TGGTGTTGTA TGCCTITAAA TCTGTGATGA TCCTCATATG GCCCAGTGTC AAGTTGTGCT | 4200 |
| TGTTTACAGC ACTACTCTGT GCCAGCCACA CAAACGTTTA CTTATCTTAT GCCACGGGAA | 4260 |
| GTTTAGAGAG CTAAGATTAT CTGGGGAAAT CAAAACAAAA AACAAGCAAA CAAAAAAAAA | 4320 |
| A | |

Seq ID NO: 94 Protein sequence:
Protein Accession #: NP_000035.1

| | |
|---|---|
| 1 11 21 31 41 51 | |
| \| \| \| \| \| \| | |
| MEVQLGLGRV YPRPPSKTYR GAFQNLFQSV REVIQNPGPR HPEAASAAPP GASLLLLQQQ | 60 |
| QQQQQQQQQQ QQQQQQQQET SPRQQQQQQG EDGSPQAHRR GPTGYLVLDE EQQPSQPQSA | 120 |
| LECHPERGCV PEPGAAVAAS KGLPQQLPAP PDEDDSAAPS TLSLLGPTFP GLSSCSADLK | 180 |
| DILSEASTMQ LLQQQQQEAV SEGSSSGRAR EASGAPTSSK DNYLGGTSTI SDNAKELCKA | 240 |
| VSVSMGLGVE ALEHLSPGEQ LRGDCMYAPL LGVPPAVRPT PCAPLAECKG SLLDDSAGKS | 300 |
| TEDTAEYSPF KGGYTKGLEG ESLGCSGSAA AGSSGTLELP STLSLYKSGA LDEAAAYQSR | 360 |
| DYYNFPLALA GPPPPPPPPH PHARIKLENP LDYGSAWAAA AAQCRYGDLA SLHGAGAAGP | 420 |
| GSGSPSAAAS SSWHTLFTAE EGQLYGPCGG GGGGGGGGGG GGGGGGGGGG GGEAGAVAPY | 480 |
| GYTRPPQGLA GQESDFTAPD VWYPGGMVSR VPYPSPTCVK SEMGPWMDSY SGPYGDMRLE | 540 |
| TARDHVLPID YYFPPQKTCL ICGDEASGCH YGALTCGSCK VFFKEAAEGK QKYLCASRND | 600 |
| CTIDKFRRKN CPSCRLHKCY EAGMTLGARK LKKLGNLKLQ EEGEASSTTS PTEETTQKLT | 660 |
| VSHIEGYECQ PIFLNVLEAI EPGVVCAGHD NNQPDSFAAL LSSLNBLGER QLVHVVKWAK | 720 |
| ALPGFRNLHV DDQMAVIQYS WMGLMVFAMG WRSFTNVNSR MLYFAPDLVF NEYRMHKSRM | 780 |
| YSQCVRMRHL SQEFGWLQIT PQEFLCMKAL LLFSIHIVDG LKNQKFFDEL RMNYKELDR | 840 |
| HACKRICNPT SCSRRFYQLT KLLDSVQPLA RELHQFTFDL LIKSIMVSVD FPEMMAEIIS | 900 |
| VQVPKILSGK VKPIYFHTQ | |

Seq ID NO: 95 DNA sequence
Nucleic Acid Accession #: NM_002497
Coding sequence: 135-1472 (underlined sequences correspond to start
and stop codons)

| | |
|---|---|
| 1 11 21 31 41 51 | |
| \| \| \| \| \| \| | |
| GGCACGAGTA GGGGTGGCGG GTCAGTGCTG CTCGGGGGCT TCTCCATCCA GGTCCCTGGA | 60 |
| GTTCCTGGTC CCTGGAGCTC CGCACTTGGC GCGCAACCTG CGTGAGGCAG CGCGACTCTG | 120 |
| GCGACTGGCC GGCCATGCCT TCCCGGGCTG AGGACTATGA AGTGTTGTAC ACCATTGGCA | 180 |
| CAGGCTCCTA CGGCCGCTGC CAGAAGATCC GGAGGAAGAG TGATGGCAAG ATATTAGTTT | 240 |
| GGAAAGAACT TGACTATGGC TCCATGACAG AAGCTGAGAA ACAGATGCIT GTTTCTGAAG | 300 |
| TGAATTTGCT TCGTGAACTG AAACATCCAA ACATCGTTCG TTACTATGAT CGGATTATTG | 360 |
| ACCGGACCAA TACAACACTG TACATTGTAA TGGAATATTG TGAAGGAGGG GATCTGGCTA | 420 |
| GTGTAATTAC AAAGGGAACC AAGGAAAGGC AATACTTAGA TGAAGAGTTT GTTCTTCGAG | 480 |
| TGATGACTCA GTTGACTCTG GCCCTGAAGG AATGCCACAG ACGAAGTGAT GGTGGTCATA | 540 |
| CCGTATTGCA TCGGGATCTT AAACCAGCCA ATGTTTTCCT GGATGGCAAG CAAAACGTCA | 600 |
| AGCTTGGAGA CTTTGGGCTA GCTAGAATAT TAAACCATGA CACGAGTTTT GCAAAAACAT | 660 |
| TTGTTGGCAC ACCTTATTAC ATGTCTCCTG AACAAATGAA TCGCATGTCC TACAATGAGA | 720 |
| AATCAGATAT CTGGTCATTG GGCTGCTTGC TGTATGAGTT ATGTGCATTA ATGCCTCCAT | 780 |
| TTACAGCTTT TAGCCAGAAA GAACTCGCTG GGAAAATCAG AGAAGGCAAA TTCAGGGGAA | 840 |
| TTCCATACCG TTACTCTGAT GAATTGAATG AAATTATTAC GAGGATGTTA AACTTAAAGG | 900 |
| ATTACCATCG ACCTTCTGTT GAAGAAATTC TTGAGAACCC TTTAATAGCA GATTTGGTTG | 960 |
| CAGACGAGCA AAGAAGAAAT CTTGAGAGAA GAGGGCGACA ATTAGGAGAG CCAGAAAAAT | 1020 |
| CGCAGGATTC CAGCCCTGTA TTGAGTGAGC TGAAACTGAA GGAAATTCAG TTACAGGAGC | 1080 |
| GAGAGCGAGC TCTCAAAGCA AGAGAAGAAA GATTGGAGCA GAAAGAACAG GAGCTTTGTG | 1140 |
| TTCGTGAGAG ACTAGCAGAG GACAAACTGG CTAGAGCAGA AAATCTGTTG AAGAACTACA | 1200 |
| GCTTGCTAAA GGAACGGAAG TTCCTGTCTC TGGCAAGTAA TCCAGAACTT CTTAATCTTC | 1260 |
| CATCCTCAGT AATTAAGAAG AAAGTTCATT TCAGTGGGGA AAGTAAAGAG AACATCATGA | 1320 |
| GGAGTGAGAA TTCTGAGAGT CAGCTCACAT CTAAGTCCAA GTGCAAGGAC CTGAAGAAAA | 1380 |
| GGCTTCACGC TGCCCAGCTG CGGGCTCAAG CCCTGTCAGA TATTGAGAAA AATTACCAAC | 1440 |
| TGAAAAGCAG ACAGATCCTG GGCATGCGCT AGCCAGGTAG AGAGACACAG AGCTGTGTAC | 1500 |
| AGGATGTAAT ATTACCAACC TTTAAAGACT GATATTCAAA TGCTGTAGTG TTGAATACTT | 1560 |
| GGCCCCATGA GCCATGCCTT TCTGTATAGT ACACATGATA TTTCGGAATT GGTTTTACTG | 1620 |
| TTCTTCAGCA ACTA1TGTAC AAAATGTTCA CATTTAATTT TTCTTTCTTC TTTTAAGAAC | 1680 |
| ATATTATAAA AAGAATACTT TCTTGGTTGG GCTTTTAATC CTGTGTGTGA TTACTAGTAG | 1740 |
| GAACATGAGA TGTGACATTC TAAATCTTGG GAGAAAAAAT AATATTAGGA AAAAAATATT | 1800 |
| TATGCAGGAA GAGTAGCACT CACTGAATAG TTTTAAATGA CTGAGTGGTA TGCTTACAAT | 1860 |
| TGTCATGTCT AGATTAAATT TTTAAGTCTG AGATTTTAAA TGTTTTTGAG CTTAGAAAAC | 1920 |
| CCAGTTAGAT GCAATTTGGT CATTAATACC ATGACATCTT GCTTATAAAT ATTGCATTGC | 1980 |
| TCTGTAGTTC AAATCTGTTA GCTTTGTGAA AATTCATCAC TGTGATGTTT GTATTCTTTT | 2040 |
| TTTTTTTCTG TTTAACAGAA TATGAGCTGT CTGTCATTTA CCTACTTCTT TCCCACTAAA | 2100 |
| TAAAAGAATT CTTCAGTTA | |

Seq ID NO: 96 Protein sequence:
Protein Accession #: NP_002488

| | |
|---|---|
| 1 11 21 31 41 51 | |
| \| \| \| \| \| \| | |
| GIREFNPNIM ANEVERINMI TGSISGENEA RELATEDKIN ASEHGMGSAP IENSMPSRAE | 60 |
| DYEVLYTLGT GSYGRCQKIR RKSDGKILVW KELDYGSMTE AEKQMLVSEV NLLRELKHPN | 120 |
| IVRYYDRIID RTNTTLYIVM EYCEGGDLAS VITKGTKBRQ YLDEEFVLRV MTQLTHALKE | 180 |
| CHRRSDGGHT VLHRDLKPAN VFLDGKQNVK LGDFGLARIL NHDTSFAKTF VGTPYYMSPE | 240 |
| QMNRMSYNEK SDIWSLGCLL YELCALMPPF TAFSQKELAG KIREGKERRI PYRYSDELNE | 300 |
| IITRMLNLKD YHRPSVEEIL ENPLIADLVA DEQRRNLERR GRQLGEPEKS QDSSPVLSEL | 360 |
| KLKEIQLQER ERALKAREER LEQKEQELCV RERLAEDKLA RAENLLKNYS LLKERKFLSL | 420 |
| ASNPELLNLP SSVIKKKVHF SGESKENIMR SENSESQLTS KSKCKDLKKR LHAAQLRAQA | 480 |
| LSDIEKNYQL KSRQILGMR | |

Seq ID NO: 97 DNA sequence
Nucleic Acid Accession #: NM_007050.2
Coding sequence: 185-4576 (underlined sequences correspond to start
and stop cudons)

| | |
|---|---|
| 1 11 21 31 41 51 | |
| \| \| \| \| \| \| | |
| CCTCCCGCCT CAGTTCGCGC CGCGCCTCGG CTTGGAACGC AGGAGCGCCG GCTCCGGGAG | 60 |
| CCCGAGCGGA GCCAGCCGCG CGCACAGCCA GCGGCCGCGC CGGCGATGCG GGGCCACCCC | 120 |
| GCGCCCGCCC CAGTCCCGGC CCCGGCCCCC GCGGGAAGGG GCTGAGCTGC CCGCCGCCGC | 180 |
| CCGGATGGCG AGCCTCGCCG CGCTCGCCCT CAGCCTGCTC CTGAGGCTGC AGCTGCCGCC | 240 |
| ACTGCCCGGC GCCCGGGCTC AGAGCGCCCC AGGTGGCTGT TCCTTTGATG AGCACTACAG | 300 |
| CAACTGTGGT TATAGTGTGG CTCTAGGGAC CAATGGGTTC ACCTGGGAGC AGATTAACAC | 360 |
| AACGGAGAAA CCAATGCTGG ACCAGGCAGT GCCCACAGGA TCTTTCATGA TGGTGAACAG | 420 |
| CTCTGGGAGA GCCTCTGGCC AGAAGGCCCA CCTTCTCCTG CCAACCCTGA AGGAGAATGA | 480 |
| CACCCACTGC ATCGACTTCC ATTACTACTT CTCCAGCCGT GACAGGTCCA GCCCAGGGGC | 540 |
| CTTGAACGTC TACGTGAAGG TGAATGGTGG CCCCCAAGGG AACCCTGTGT GGAATGTGTC | 600 |
| CGGGGTCGTC ACTGAGGGCT GGGTGAAGGC AGAGCTCGCC ATCAGCACTT TCTGGCCACA | 660 |
| TfTCTATCAG GTGATATTTG AATCCGTCTC ATTGAAGGGT CATCCTGGCT ACATCGCCGT | 720 |
| GGACGAGGTC CGGGTCCTTG CTCATCCATG CAGAAAAGCA CCTCATTTTC TGCGACTCCA | 780 |
| AAACGTGGAG GTGAATGTGG GGCAGAATGC CACATTTCAG TGCATTGCTG GTGGGAAGTG | 840 |
| GTCTCAGCAT GACAAGCTTT GGCTCCAGCA ATGGAATGGC AGGGACACGG CCCTGATGGT | 900 |
| CACCCGTGTG GTCAACCACA GGCGCTTCTC AGCCACAGTC AGTGTGGCAG ACACTGCCCA | 960 |
| GCGGAGCGTC AGCAAGTACC GCTGTGTGAT CCGCTCTGAT GGTGGGTCTG GTGTGTCCAA | 1020 |
| CTACGCGGAG CTGATCGTGA AAGAGCCTCC CACGCCCATT GCTCCCCCAG AGCTGCTGGC | 1080 |
| TGTGGGGGCC ACATACCTGT GGATCAAGCC AAATGCCAAC TCCATCATCG GGGATGGCCC | 1140 |
| CATCATCCTG AAGGAAGTGG AATATCGCAC CACCACAGGC ACGTGGGCAG AGACCCACAT | 1200 |
| AGTCGACTCT CCCAACTATA AGCTGTGGCA TCTGGACCCC GATGTTGAGT ATGAGATCCG | 1260 |
| AGTGCTCCTC ACACGACCAG GTGAGGGGGG TACGGGACCG CCAGGGGCTC CCCTCACCAC | 1320 |
| CAGGACCAAG TGTGCAGATC CGGTACATGG CCCACAGAAC GTGGAAATCG TAGACATCAG | 1380 |
| AGCCCGGCAG CTGACCCTGC AGTGGGAGCC CTTCGGCTAC GCGGTGACCC GCTGCCATAG | 1440 |
| CTACAACCTC ACCGTGCAGT ACCAGTATGT GTTCAACCAG CAGCAGTACG AGGCCGAGGA | 1500 |
| GGTCATCCAG ACCTCCTCCC ACTACACCCT GCGAGGCCTG CGCCCCTfCA TGACCATCCG | 1560 |
| GCTGCGACTC TfGCTGTCTA ACCCCGAGGG CCGAATGGAG AGCGAGGAGC TGGTGGTGCA | 1620 |
| GACTGAGGAA GACGTTCCAG GAGCTGTTCC TCTAGAATCC ATCCAAGGGG GGCCCTTTGA | 1680 |
| GGAGAAGATC TACATCCAGT GGAAACCTCC CAATGAGACC AATGGGGTCA TCACGCTCTA | 1740 |
| CGAGATCAAC TACAAGGCTG TCGGCTCGCT GGACCCAAGT GCTGACCTCT CGAGCCAGAG | 1800 |
| GGGGAAAGTG TTCAAGCTCC GGAATGAAAC CCACCACCTC TTTGTGGGTC TGTACCCAGG | 1860 |
| GACCACCTAT TCCTTCACCA TCAAGGCCAG CACAGCAAAG GGCTTTGGGC CCCCTGTCAC | 1920 |
| CACTCGGATT GCCACCAAAA TTTCAGCTCC ATCCATGCCT GAGTACGACA CAGACACCCC | 1980 |
| ATTGAATGAG ACAGACACGA CCATCACAGT GATGCTGAAA CCCGCTCAGT CCCGGGGAGC | 2040 |
| TCCTGTCAGT GTTTATCAGC TGGTTGTCAA GGAGGAGCGA CTTCAGAAGT CACGGAGGGC | 2100 |
| AGCTGACATT ATTGAGTGCT TTTCGGTGCC CGTGAGCTAT CGGAATGCCT CCAGCCTCGA | 2160 |
| TTCTCTACAC TACTTTGCTG CTGAGTTGAA GCCTGCCAAC CTGCCTGTCA CCCAGCCATT | 2220 |
| TACAGTGGGT GACAATAAGA CATACAATGG CTACTGGAAC CCTCCTCTCT CTCCCCTGAA | 2280 |
| AAGCTACAGC ATCTACTTCC AGGCACTCAG CAAAGCCAAT GGAGAGACCA AAATCAACTG | 2340 |
| TGTTCGTCTG GCTACAAAAG CACCAATGGG CAGCGCCCAG GTGACCCCGG GGACTCCACT | 2400 |
| CTGCCTCCTC ACCACAGGTG CCTCCACCCA GAATTCTAAC ACTGTGGAGC CAGAGAAGCA | 2460 |
| GGTGGACAAC ACCGTGAAGA TGGCTGGCGT GATCGCTGGC CTCCTCATGT TCATCATCAT | 2520 |
| TCTCCTGGGC GTGATGCTCA CCATCAAAAG GAGAAGAAAT GCTTATTCCT ACTCCTATTA | 2580 |
| CTTGTCCCAA AGGAAGCTGG CCAAGAAGCA GAAGGAGACC CAGAGTGGAG CCCAGAGGGA | 2640 |
| GATGGGGCCT GTGGCCTCTG CCGACAAACC CACCACCAAG CTCAGCGCCA GCCGCAATGA | 2700 |
| TGAAGGCTTC TCTTCTAGTT CTCAGGACGT CAACGGATTC ACAGATGGCA GCCGCGGGGA | 2760 |
| GCTTTCCCAG CCCACCCTCA CGATCCAGAC TCATCCCTAC CGCACCTGTG ACCCTGTGGA | 2820 |
| GATGAGCTAC CCCCGGGACC AGTTCCAACT CGCCATCCGG GTGGCTGACT TGCTGCAGCA | 2880 |
| CATCACGCAG ATGAAGAGAG GCCAGGGCTA CGGGTTCAAG GAGGAATACG AGGCCTTACC | 2940 |
| AGAGGGGCAG ACAGCTTCGT GGGACACAGC CAAGGAGGAT GAAAACCGCA ATAAGAATCG | 3000 |
| ATATGGGAAC ATCATATCCT ACGACCATTC CCGGGTGAGG CTGCTGGTGC TGGATGGAGA | 3060 |
| CCCGCACTCT GACTACATCA ATGCCAACTA CATTGACGGA TACCATCGAC CTCGGCACTA | 3120 |
| CATTGCGACT CAAGGTCCGA TGCAGGAGAC TGTAAAGGAC TTTTGGAGAA TGATCTGGCA | 3180 |
| GGAGAACTCC GCCAGCATCG TCATGGTCAC AAACCTGGTG GAAGTGGGCA GGGTGAAATG | 3240 |
| TGTGCGATAC TGGCCAGATG ACACGGAGGT CTACGGAGAC ATTAAAGTCA CCCTGATTGA | 3300 |
| AACAGAGCCC CTGGCAGAAT ACGTCATACG CACCTTCACA GTCCAGAAGA AAGGCTACCA | 3360 |
| TGAGATCCGG GAGCTCCGCC TCTTCCACTT CACCAGCTGG CCTGACCACG GCGTTCCCTG | 3420 |
| CTATGCCACT GGCCTTCTGG GCTTCGTCCG CCAGGTCAAG TTCCTCAACC CCCCGGAAGC | 3480 |
| TGGGCCCATA GTGGTCCACT GCAGTGCTGG GGCTGGGCGG ACTGGCTGCT TCATTGCCAT | 3540 |
| TGACACCATG CTTGACATGG CCGAGAATGA AGGGGTGGTG GACATCTTCA ACTGCGTGCG | 3600 |
| TGAGCTCCGG GCCCAAAGGG TGAACCTGGT ACAGACAGAG GAGCAATATG TGTTTGTGCA | 3660 |
| CGATGCCATC CTGGAAGCGT GCCTCTGTGG CAACACTGCC ATCCCTGTGT GTGAGTTCCG | 3720 |
| TTCTCTCTAC TACAATATCA GCAGGCTGGA CCCCCAGACA AACTCCAGCC AAATCAAAGA | 3780 |
| TGAATTTCAG ACCCTCAACA TTGTGACACC CCGTGTGCGG CCCGAGGACT GCAGCATTGG | 3840 |
| GCTCCTGCCC CGGAACCATG ATAAGAATCG AAGTATGGAC GTGCTGCCTC TGGACCGCTG | 3900 |
| CCTGCCCTTC CTTATCTCAG TGGACGGAGA ATCCAGCAAT TACATCAACG CAGCACTGAT | 3960 |
| GGATAGCCAC AAGCAGCCTG CCGCCTTCGT GGTCACCCAG CACCCTCTAC CCAACACCGT | 4020 |
| GGCAGACTTC TGGAGGCTGG TGTTCGATTA CAACTGCTCC TCTGTGGTGA TGCTGAATGA | 4080 |
| GATGGACACT GCCCAGTTCT GTATGCAGTA CTGGCCTGAG AAGACCTCCG GGTGCTATGG | 4140 |
| GCCCATCCAG GTGGAGTTCG TCTCCGCAGA CATCGACGAG GACATCATCC ACAGAATATT | 4200 |
| CCGCATCTGT AACATGGCCC GGCCACAGGA TGGTTATCGT ATAGTCCAGC ACCTCCAGTA | 4260 |
| CATTGGCTGG CCTGCCTACC GGGACACGCC CCCCTCCAAG CGCTCTCTGC TCAAAGTGGT | 4320 |
| CCGACGACTG GAGAAGTGGC AGGAGCAGTA TGACGGGAGG GAGGGACGTA CTGTGGTCCA | 4380 |
| CTGCCTAAAT GGGGGAGGCC GTAGTGGAAC CTTCTGTGCC ATCTGCAGTG TGTGTGAGAT | 4440 |
| GATCCAGCAG CAAAACATCA TTGACGTGTT CCACATCGTG AAAACACTGC GTAACAACAA | 4500 |
| ATCCAACATG GTGGAGACCC TGGAACAGTA TAAATTTGTA TACGAGGTGG CACTGGAATA | 4560 |
| TTTAAGCTCC TTTTAGCTCA ATGGGATGGG GAACTGCCGG AGTCCAGAGG CTGCTGTGAC | 4620 |
| CAAGCCCCCT TTTGTGTGAA TGGCAGTAAC TGGGCTCAGG AGCTCTGAGG TGGCACCCTG | 4680 |
| CCTGACTCCA AGGAGAAGAC TGGTGGCCCT GTGTTCCACG GGGGGCTCTG CACCTTCTGA | 4740 |
| GGGGTCTCCT GTTGCCGTGG GAGATGCTGC TCCAAAAGGC CCAGGCTTCC TTTTCAACCT | 4800 |
| AACCAGCCAC AGCCAAGGGC CCAAGCAGAA GTACACCCAC AAGCAAGGCC TTGGATTTCT | 4860 |
| GGCTCCCAGA CCACCTGCTT TTGTTCTGAG TTTGTGGATC TCTTGGCAAG CCAACTGTGC | 4920 |
| AGGTGCTGGG GAGTGGGAGG CTCCCCTGCC CTCCTTCTCC TTAGGAGTGG AGGAGATGTG | 4980 |
| TGTTCTGCTC CTCTACGTCA TGGAAAAGAT TGAGGCTCTT GGGGGTCACT GCTCTGCTGC | 5040 |
| CCCCTGCAAC CTCCTTCAGG GGCCTCTGGC ACCAGACATT TGCAGTCTGG ACCAGTGTGA | 5100 |
| CTTTACGATG TTCCCTAGGC CACAAGAGAG GCCCCCCATC CTCACACCTA ACCTGCATGG | 5160 |
| GGCTTCGCCC ACAACCATTC TGTACCCCTT CCCCAGCCTG GGCCTTGACC GTCCAGCATT | 5220 |
| CACTGGCCGG CCAGCTGTGT CCACAGCAGT TTTTGATAAA GGTGTTCTTT GCTTTTTTGT | 5280 |
| GTGGTCAGTG GGAGGGGGTG GAACTGCAGG GAACTTCTCT GCTCCTCCTT GTCTTTGTAA | 5340 |
| AAAGGGACCA CCTCCCTGGG GCAGGGCTTG GGCTGACCTG TAGGATGTAA CCCCTGTGTT | 5400 |
| TCTTTGGTGG TAGCTTTCTT TGGAAGAGAC AAACAAGATA AGATTTGATT ATTTTCCAAA | 5460 |
| GTGTATGTGA AAAGAAACTT TCTTTTGGAG GGTGTAAAAT CTTAGTCTCT TATGTCAAAA | 5520 |
| AGAAGGGGGC GGGGGAGTTT GAGTATGTAC CTCTAAGACA AATCTCTCGG GCCTTTTATT | 5580 |
| TTTTCCTGGC AATGTCC3TA AAAGCTCCCA CCCTGGGACA GCATGCCACT GAGCAAGGAG | 5640 |
| AGATGGGTGA GCCTGAAGAT GGTCCCTTTG GTTTCTGGGG CAAATAGAGC ACCAGCTTTG | 5700 |
| TGCATAATTT GGATGTCCAA ATTTGAACTC CTTCCTAAAG AAACCCAGCA GCCACCTTGA | 5760 |
| AAAAGGCCAT TGTGGAGCCC ATTATACTTT GATTTAAAAT AGGCCAAGAG AATCAGGCCT | 5820 |
| GGAGATCTAG GGTCTTGTCC AAAGTGTGAG TGAGTCAATG AGAGGGAACC AACATTTGCT | 5880 |
| AAGTCTCTAC TGTATGCCAG GGATCATGCT TGGCACTTTC CATAGGACAT TTCACACAGT | 5940 |
| CCTTAGAACC CCCAGGAGAG AGCTACTGAC TTGTTATCAT CTCCATTTGA TCATCTCCTC | 6000 |
| CAATGAGGAA ACCCACGCAC CTTCCTTAGT AATGAAATCC TGGGTTCCAA AGGGGCAGGT | 6060 |
| AATGGCAATG AGACTTCTCC GTGCTGT1TT CTTCATCTTC TCTAAGCCAA GCAATTATTT | 6120 |
| TATGGAGGGA AAATAAGGCC AGAAACTTCT GAGCAGATAA CTCCACAAAT GGAAATTTAG | 6180 |
| TACTTTCTTC CTGATGCCAG TTCTTCTGGG AAGCGCAGAA TTTCAGATAT ATTTTAGTAA | 6240 |
| CACATTCCCA GCTCCCCAGG AAAGCCAGTC TCATCTAATT TCTTAGTCAG TAAAAACAAT | 6300 |
| TCCCTGTTCC TTCAGGCTAT GAATGGACCA GCCAGGGAAA CTCTCGACCT TGATCTCTAG | 6360 |
| CCAGTGCTTA GGCCCAATAT CTGACAGCCT CAGGTGGGCT GGGACCTAGG AAGCTCCATC | 6420 |
| TTGAAGGCTG GTCTAGCCCC AGACAGGGCA TGAGGGGCAG AGAATTCAAG AAGGTACAGC | 6480 |
| TTTGGCCCTC AAGAGCCCAC TGTATGCTGG GGAAATGGAA CCATGGTGCA GTAGTGTGGA | 6540 |
| GTGGATGAGT GTTCCATGAG CCTAGGAGCA AGAAAGTCTC TTCGGCCTCG GGCTTCCTGG | 6600 |
| AGAAGGGGAC GTCCATTCCT GCTGGGTCTT AACAAGCATA AAAAGGAAAA AAAGGAAACT | 6660 |
| CAGGCAAAGG GATCCATATG TGCAATGGCA AAGAAATGTG AAAAGGCATT GGGAGAAGCA | 6720 |
| GTCTGGGGGA GGCCAGCCCA GTGCGGGCAC AGCACAACAC GGGGAGCAGC AAGAGATGAG | 6780 |
| CCAGGGTCCA GGAGACAGAT GCCCATCGCG AGTACAGACT TTGTCCTATT GGCAACAAGG | 6840 |
| AGTCCATGGA GCTTTAGAGA GATGCACTCA GCTTCGTGTT GGCCAAGACT CCTTCTGGGC | 6900 |
| CAATGGGGCT GCCTCTTTTC CTTTCATCAG ACACTGTGAA AACATTCCCT TAAGCGTGCA | 6960 |
| CTTTTTAATA TCACATCTAT TTGTCTGTCT GCTCATTGTT TTGTTGCTGG AACTAAATAT | 7020 |
| GCAATGGATC ATGAGACTCA GATTCTATGA GAAACCCAGG GTCTCTGCTT TACCACGGAG | 7080 |
| CAGGGTCACC AACCCAGATC TCCAGGCCCA TGAGGATGGA ACATGAAAGG AGCCGACAAA | 7140 |
| AGTTGCTTCC ATTGGCATGG GCTCTGGAGC TGTCCAGAAG TCCAGGGACA CCAGACTTGA | 7200 |
| TCAAGGAAGG GCTGTCACTT TAGAGGTTCA AAAGGAAGTG CCTCAAAGCA AACGCAAGCA | 7260 |
| AAGGAACCCC ACGATGAACT TGCTCTTTTC CTTTGATGAG CCTCTCCCCA GGTGTATTTC | 7320 |
| AGCAGACCCC GGGGACCCAC CCCCACTGGG CCTGCTGCCC TCCCTCGGCT CCAGCCCAAT | 7380 |
| GCCCCAGCTG CCCTTCCCCA GCCTGCAACG AGCCTGTAGC ATGGCAAATC TGCCTGCTGT | 7440 |
| ATGCTATTTT CTTAGATCTT GGTACATCCA GACAGGATGA GGGTGGAGGG AGAGCTATTT | 7500 |
| AACACAAATC CTAAGATTTT TTTCTGCTCA GQAAGGGGTG AAATAGCTGG CAGATACAAA | 7560 |
| AGACAGTGGC TITTATCATT TTAAATGGTA GGAATTTAAG GTGTGACTTC AGGGAGAAAC | 7620 |
| AAACTTGCAA AAAAAAAAAA TCTCAGGCCA TGTTGGGGTA ACCCAGCAAG GGCCAGTGAT | 7680 |
| GATTTCCCCC AGCTCATCCC CTTATITTCC CACAACCCAA CCATTCTCTA AAGCAGGACA | 7740 |
| GTGAATAGGT CTTAGGCCAG TGCACACAGG AAGAAATTGA GGCTTATGGA TGGGGATGAC | 7800 |
| TTCCCTAAGA TCCCATGGGA CAAGGATGTG GCAAGGCTTG GATGAGATGG GGCACCAGTG | 7860 |
| CCCAGGAATT TGAACATTTT CCTTTACCCA GGAAATCTCC GGAGCCAACA CCACCACCCC | 7920 |
| CAGGGGGTCT CCCCACCCCA CCCCATTTAC AGGGTGAGCT CAGCCTGTCA TGAGCAGAGG | 7980 |
| AAAATATTAT TAATGCTCTC TGAGTCTTTA CAACAGGAGC TCTTACCTCA TAGATGTGGG | 8040 |
| CTCTGTTTGG GGAAGATGCA AGGAAGTAAT GAGAAGCCCA GGAAATTTCT CCACCTGTGT | 8100 |
| TTATGGCCTA AATAGCTTCA GGATGTATCT TAGCTGCACT CCAACATTGC ATCCTTTCTG | 8160 |
| GGGTGAAGAA TCTGGGCCAA CCAGGGGTCC TTGGGCCTCT AGAAGGCCAC AGTAGGCCTC | 8220 |
| TCTTTGTGGG AATGGAAGGG GACAGTTTGC TTTTAGTGCT GGCCCTCTCT GTGGGTGTGG | 8280 |
| CCTGCAAAGG AACCAACAGA CCCTATGCTG GGGACTCTAA CATGTGAGCT CATTAAATTC | 8340 |
| TTCCAGCATT CTAAAGGAGG GTTTGTGATT GTCACCATTT ACTGATGAGG AAACTAAGGC | 8400 |
| TCCTAGGGGA GAAATCACTT GCCCACAGTT CCACAGCTAG TGAGTGAATG AACCAGGATT | 8460 |
| TAAACCGGTT TTTTCTCACT ACAGAGACAA TATTTITCCA CCATTGTATC TCACATTTTT | 8520 |
| CCCAGGAGGT TACCCATAAC AGAAGAGACT AGAGTGGAAC AGATACGTCA GTGGATAAAG | 8580 |
| CTCAAAGCAA ACAACAGTAA GCTTAAAATT CCTTCATAGT CTCATGTTTT ACGTTCACAA | 8640 |
| TTCATGCAAA ATTTCCATTC CACTTTCTGA TTTAGCCTTG TTGGTTTTAA TATGACTCTA | 8700 |
| TGAATATTTC AAAAAAAAAT GTGCTCTGTT CCTCATGTTG TTCTGTTCTG TTCACCCCGC | 8760 |
| TATGACGGAC CCTAGGTCAG CTGGTCTTCA GCTTGACCCT AGAATTGACT CTAGGAGCAG | 8820 |
| TGACCCTGCT GCCTCCCAGA GCCAGTTATA GGCTCAAGAT CAAGACCAAC TGACCTTCTC | 8880 |
| CTAGGCAGCT CCTTTGGTGT GTGGGTGCTC TGACCTCACT GTTCATGAGG GGACCTCAAC | 8940 |
| TAAGGCATCT TCCACTTGGG TGCTGGAAGG AACCCATTAA CTCACACTAG AATGATGAGG | 9000 |
| ATTTGCTCAT CTGGCGTGGA GAAGGATGAG CCCACAAAAC CCTAAAGGGA AAAGAGAAGC | 9060 |
| TGGACACAGC TGTACTCAGC AGATTCCTGA ATGCTAGGCT GGAAAGTGGT GCCTGTTGTC | 9120 |
| CAAGTGGAGT CACATGGTTG CTAATGTGGG CAAGTCTGAG GACACAC1TC ATGAGCAGCT | 9180 |
| GGGGTCTGGA AGGCTCCTCA CTTTACCCTA GCCACACATA ATTACTGGGT GCCTACAGCA | 9240 |
| CCTAGCACCT TGGAGGGGGC ACTATTAGGA AATCGAGATT ACTATGGCAC AATTAATTCC | 9300 |
| TGGGTAAGGC ATGGGGITGT GGTGGACAGA GCTCAGTCTT TAGTTTGAAC GAAAACATAC | 9360 |
| ATACATGAAA AACATACATG AAAAAAGGAC CCTCATCAAC ATTAGAAGGG GTAGATTTGG | 9420 |
| AGCACTTTAG GCAGGAAAAC AGGAACGCAA GGCCAGGAAA CTGGAACCCA GTGAATACTC | 9480 |
| AGAACCGAGG ATGCAGATGA CTTATTTAGC AAAATGGTCA CTTCTGTGAC ATAGCTGGAG | 9540 |
| AAAGGATGGG TAACAGCTTG CCAGAGCCAC TTGGAACAAG GGCAAATCTC AGTGTCTGGG | 9600 |
| GCAAAAGATG ATGCATTTCC CTCTGACCCA TCATGTTTAT TCATCCTCCA CTCCCCATTG | 9660 |
| CCACACTAGC TCTTGCTGTA AGTCCTCACC AGGATCTACA TTTCCTCGTC GCTGGTGGGA | 9720 |
| ACCCCTTAGA GTACATAGAG GTATCAGTCC AGTAAGACTG CTCTACACAA CAGAAGTGAG | 9780 |
| GCCCAGGGAG TAGCAGCCAG GCCCTTATCC TGTTACCTCT GCAGGAGTGA CTGCCCAACC | 9840 |
| CAGATCCAGA GACATTGAAG GAAATGATAA TTCCTTGGTA CCTCACTGCC TTGGGACAAA | 9900 |
| ATGAAGAAAG CCACCCTTCC TTAGGCTGCA GCTTGCCACT CCTGGGCTGG GTAAACAGGT | 9960 |
| CATCAGCACC AGGCTCAACC AGGAGTAACA TTCTGGAAGA CATGGGTGAG CCCAAGAGGA | 10020 |
| AGCATGAACA GGACGCTGTT CCTAAGTCAT GTCAACAGGT TGTGCTGGGC CAGGATCCCC | 10080 |
| AGGGAAAAAA ATGGTCAACC CAACTGGAGG GTAGGTTAGA AGAAAAAAAA CATAAACGTG | 10140 |
| GATAGTCATG TCATCTCAAA TCCCTGACTT GGCTTCCCCA TTACTTGACA GTCTGAGCTC | 10200 |
| CTTCTTAGCC TGTGACCAGC TTCAAATCAC AGCCAAGTAA AACAAGGAAA TAGGAAAAGT | 10260 |
| AAATCCAACT AGAAGAGACA AGCTGAGATT CAGATTTGTT TACTCCTCCC ATGCAAAGTT | 10320 |
| TCCCTGTTGG AGGTTTfCCA TGTATACATG TCTAGAAGTG ATAGAATGCA AGGCCTTGGC | 10380 |
| TTTGTCTTGC AGGGATCTGC CTTTGAGGTC ATAGACTGAA CAGCAGGGAG AGAGGTTAGT | 10440 |
| GGTGGAGTGT GGGGGGAGCT GTTCTAGCTC CAGTTTCTTC TGACACATTT TTCAGGATCA | 10500 |
| TGGATCTGAT CCTCCGAAGC ACAGCAGAGA TATCTAAGCC ATATTTGTGC ACATGAGCAG | 10560 |
| ACTCTTCTAG TTTTTTAGTA ACCAGGGATG TGCTTTTGCA TGGCACTGAC TATAGAGATG | 10620 |
| TCTTGTAGAG ATCAAGCCAG TCTTTTGCAT CCCACCTGCC CACCTCCAGA AGAGATGGGA | 10680 |
| AAAGGTCATC AAAGGGCATT CACCAACTGA AATCCACTCA TGAATGTTAG GTCTCTAAAA | 10740 |
| GGAGGCATCA ACACTCACAA TGGTAGCCTC CAAACCTAGC ATCCCACCTA TCTAAGAGCT | 10800 |
| CAGGGGTGGT CCACTGGGGC AGATACAAGG GAAGTGCAAG GGCTCAGGAT GAAAGAAAAT | 10860 |
| CTATTGGGAA GAGTTTTAGG GGCTTGATCA TTATGGGGCT TCCTTCTATA TCTGAGAACT | 10920 |
| GCTCTGGGTG GTGAGATGTG GACTCTGATC CTTAATTGGA ATGTTCGGAG AATGAGTGTC | 10980 |
| TGGTGGCCTT GAAGTGTTGG ACAGAAAAGT ATCAGTATAA AAGCCTGGAG CTCAGGGTAA | 11040 |
| TTAATGTAGT TCATGGTTCC TTAGTGAGCA GGACTCTTTG ATGTGGAGGA GAAAGGGTCA | 11100 |
| TAGGAAGTAA ACCACCAAAA TTACAAAATT GAGTCTCTGT ACAATTACTT CAGTGCCTTT | 11160 |
| GGGCTTATGA ATACAAATCA GTGGGCCTTC TCTATGATGG TCCAAGAAAC TCTCAGTGTC | 11220 |
| CACCCTGTCC CTGTATCTCC CATGGAAGAT GAATAATGTC AGGTGTTCTT TGGGTCAAAG | 11280 |
| GCCCCAGGGC AGTCTGGAGG CTTAGAGGGC AGAGTGGTGT CATTCCATGT AAAGTTAGGC | 11340 |
| TTCTGAGGGG TCAGGCAGAA TATGGTGTCC ATATCTTCCA TAGCTCTGCA GATTCTTGGA | 11400 |
| TGAAGTCAAG CACAGTTTGC TAGACCCAGG TCACTCCTCT GAGTATAACT AGGACCCATG | 11460 |
| AGTGAAACTT AATAGCTGTA AGGAAGAACC TGCTGTCTGC CAGAGAGGAT AAGCTGCCCA | 11520 |
| TCTCAGCAGC TGTCTAAAAG AAGGCAGGTG TCTCTTTAAA GGGAAGAGAA GCATTGGTGA | 11580 |
| AATGGATTTC AGGTCACTTC CATTCCAGAT GGGTGAGATC TTGTGGAGCT GGGATCATGT | 11640 |
| TTGAACTCAT TCATACCTGT AGAGCACGAA TCCAAGTAGA TTGTGTTTGG TCTGTACAGG | 11700 |
| CTGAAGCCCC CTGCTCTCCC ACCCAAGTGC CCCCACTGAG CAGGCCAACA TGCTGTTGTG | 11760 |
| GCCACATATA CTGGGCTGAT CCAGGCTGGT TATCACCAAA CAGCAAACCA TAGGGAACAG | 11820 |
| CTGCTTTGCC ATAGACCCAA TACCCATGTA GATCTCTCAT GAGAGCAGCC ATAACTCAGA | 11880 |
| CCCACTGACC AACAGGGCCA TGAGTGACAG CCAGAACCAG TGAAGGTCCA AGTAGGACAC | 11940 |
| AGAGCAGGGC TTTTCTTACC ATACAGATTA TCTCCAGAGG TTATTTCTAC CCCACTCCCT | 12000 |
| ATTCAAGGCC TGTTGGAGCA CACTGCAAAA GCAAAAGCAC AGTAACTCAA TTTACACATG | 12060 |
| ATTATAATCA TTTCCAGTGC ACACATTTCA TCACCAGGTG GATCCTGAGC TAGCCCATGT | 12120 |
| AAATCCGGGT TAACCCATAT TGGTAATCAT ACTCAAAAGC ACTTTTCACC CTACATTCTA | 12180 |
| CTAGCCAATC AAAGACAAAG AGTTGTGGCC TCTACCATTG CCTTGGCTTC TGGACACCCT | 12240 |
| CACAAGCTAT CCCAAGGTTC CCGCTCAACT CCAGGGAGGC TGACATCTTC ACATCCACTG | 12300 |
| GGCATATAAT ATTGCATGAG ACCAAAGTCT CCACACTCTT TGCAGCCTCC TCCATGAATC | 12360 |
| CCAATGGCCT GCACTTGTAC AGTTTGGGTG TTTGATAGAT AAAGCACGTA TGAGAAGAGA | 12420 |
| AAACAAAATA AATCAACTTT TTAAAAAAGC CAGCACTGTG CTGTCAATGT TTTTTTTTTC | 12480 |
| TTTTCAATTC TAGCTCAGAA AAGCAGAAGG TAAATAATGT CAGGTCAATG AATATCAGAT | 12540 |
| ATATTTTTTG ACTGTACATT ACAGTGAAGT GTAATCTTTT TACACCTGCA AGTCCATCTT | 12600 |
| ATTTATTCTT GTAAATGTTC CCTGACAATG TTTGTAATAT GGCTGTGTTA AAAAATCTAT | 12660 |
| ACAATAAAGC TGTGACCCTG | |

Seq ID NO: 98 Protein sequence:
Protein Accession #: NP_008981.1

| | |
|---|---|
| 1 11 21 31 41 51 | |
| \| \| \| \| \| \| | |
| GIREFNPPRG TEINTYRGSI NEPHGSPHAT ASERECEPTG RTYPETHGMG SAPIENSMAS | 60 |
| LAALALSLLL RLQLPPLPGA RAQSAPGGCS FDEHYSNCGY SVALGTNGFT WEQINTTEKP | 120 |
| MLDQAVPTGS FMMVNSSGRA SGQKAHLLLP TLKENDTHCI DFHYYFSSRD RSSPGALNVY | 180 |
| VKVNGGPQGN PVWNVSGVVT EGWVKAELAI STFWPHFYQV IFESVSLKGH PGYIAVDEVR | 240 |
| VLAHPCRKAP HFLRLQNVEV NVGQNATFQC IAGGKWSQHD KLWLQQWNGR DTALMVTRVV | 300 |
| NHRRFSATVS VADTAQRSVS KYRCVIRSDG GSGVSNYAEL IVKBPPTPIA PPELLAVGAT | 360 |
| YLWIKPNANS IIGDGPIILK EVEYRTTTGT WAETHIVDSP NYKLWHLDPD VEYEIRVLLT | 420 |
| RPGEGGTGPP GAPLTTRTKC ADPVHGPQNV EIVDIRARQL TLQWEPFGYA VTRCHSYNLT | 480 |
| VQYQYVFNQQ QYEAEEVIQT SSHYTLRGLR PFMTIRLRLL LSNPEGRMES EELVVQTEED | 540 |
| VPGAVPLESI QGGPFEEK1Y IQWKPPNETN GVITLYEINY KAVGSLDPSA DLSSQRGKVF | 600 |
| KLRNETHHLF VGLYPGTTYS FTIKASTAKG FGPPVTTRIA TKISAPSMPE YDTDTPLNET | 660 |
| DTTITVMLKP AQSRGAPVSV YQLVVKEERL QKSRRAADII ECFSVPVSYR NASSLDSLHY | 720 |
| EAAELKPANL PVTQPFTVGD NKTYNGYWNP PLSPLKSYSI YFQALSKANG ETKINCVRLA | 780 |
| TKAPMGSAQV TPGTPLCLLT TGASTQNSNT VEPEKQVDNT VKMAGVIAGL LMFIIILLGV | 840 |
| MLTIKRRRNA YSYSYYLSQR KLAKKQKETQ SGAQREMGPV ASADKPTTKL SASRNDEGFS | 900 |
| SSSQDVNGFT DGSRGELSQP TLTIQTHTYR TCDPVEMSYP RDQFQLAIRV ADLLQIITQM | 960 |
| KRGQGYGFKE EYEALPEGQT ASWDTAKEDE NRNKNRYGNI ISYDHSRVRL LVLDGDPHSD | 1020 |
| YINANYIDGY HRPRHYIATQ GPMQETVKDF WRMIWQENSA SIVMVTNLVE VGRVKCVRYW | 1080 |
| PDDTEVYGDI KVTLIETEPL AEYVIRTFTV QKKGYHEIRE LRLFHFTSWP DHGVPCYATG | 1140 |
| LLGFVRQVKF LNPPEAGPIV VHCSAGAGRT GCFIAIDTML DMAENEGVVD IFNCVRELRA | 1200 |
| QRVNLVQTEE QYVFVHDAIL EACLCGNTAI PVCEFRSLYY NISRLDPQTN SSQIKDEFQT | 1260 |
| LNIVTPRVRP EDCSIGLLPR NHDQIRSMDV LPLDRCLPFL ISVDGESSNY INAALMDSHK | 1320 |
| QPAAFVVTQH PLPNTVADFW RLVFDYNCSS VVMLNEMDTA QFCMQYWPEK TSGCYGPIQV | 1380 |
| EFVSADIDED IIHRIFRICN MARPQDGYRI VQHLQYIGWP AYRDTPPSKR SLLKVVRRLE | 1440 |
| KWQEQYDGRE GRTVVHCLNG GGRSGTFCAI CSVCEMIQQQ NIIDVFHIVK TLRNNXSNMV | 1500 |
| ETLEQYKFVY EVALEYLSSE | |

Seq ID NO: 99 DNA sequence
Nucleic Acid Accession #: NM_002988.1
Coding sequence: 71-340 (underlined sequences correspond to start and
stop codons)

| | |
|---|---|
| 1 11 21 31 41 51 | |
| \| \| \| \| \| \| | |
| CCGGCACGAG AGGAGTTGTG AGTTTCCAAG CCCCAGCTCA CTCTGACCAC TTCTCTGCCT | 60 |
| GCCCAGCATC ATGAAGGGCC TTGCAGCTGC CCTCCTTGTC CTCGTCTGCA CCATGGCCCT | 120 |
| CTGCTCCTGT GCACAAGTTG GTACCAACAA AGAGCTCTGC TGCCTCGTCT ATACCTCCTG | 180 |
| GCAGATTCCA CAAAAGTTCA TAGTTGACTA TTCTGAAACC AGCCCCCAGT GCCCCAAGCC | 240 |
| AGGTGTCATC CTCCTAACCA AGAGAGGCCG GCAGATCTGT GCTGACCCCA ATAAGAAGTG | 300 |
| GGTCCAGAAA TACATCAGCG ACCTGAAGCT GAATGCCTGA GGGGCCTGGA AGCTGCGAGG | 360 |
| GCCCAGTGAA CTTGGTGGGC CCAGGAGGGA ACAGGAGCCT GAGCCAGGGC AATGGCCCTG | 420 |
| CCACCCTGGA GGCCACCTCT TCTAAGAGTC CCATCTGCTA TGCCCAGCCA CATTAACTAA | 480 |
| CTTTAATCTT AGTTTATGCA TCATATTTCA TTTTGAAATT GATTTCTATT GTTGAGCTGC | 540 |
| ATTATGAAAT TAGTATTTTC TCTGACATCT CATGACATTG TCTTTATCAT CCTTTCCCCT | 600 |
| TTCCCTTCAA CTCTTCGTAC ATTCAATGCA TGGATCAATC AGTGTGATTA GCTTTCTCAG | 660 |
| CAGACATTGT GCCATATGTA TCAAATGACA AATCTTTATT GAATGGTTTT GCTCAGCACC | 720 |
| ACCTTITAAT ATATTGGCAG TACTTATTAT ATAAAAGGTA AACCAGCATT CTCACTGTGA | 780 |
| AAAAAAAAAA AAAAAAAAAA AAA | |

Seq ID NO: 100 Protein sequence:
Protein Accession #: NP_002979.1

| | |
|---|---|
| 1 11 21 31 41 51 | |
| \| \| \| \| \| \| | |
| MKGLAAALLV LVCTMALCSC AQVGTNKELC CLVYTSWQIP QKFIVDYSET SPQCPKPGVI | 60 |
| LLTKRGRQIC ADPNKKWVQK YISDLKLNA | |

Seq ID NO: 101 DNA sequence
Nucleic Acid Accession #: NM_015507.2
Coding sequence: 241-1902 (underlined sequence correspond to start and
stop codons)

| | |
|---|---|
| 1 11 21 31 41 51 | |
| \| \| \| \| \| \| | |
| CCGCAGAGGA GCCTCGGCCA GGCTAGCCAG GGCGCCCCCA GCCCCTCCCC AGGCCGCGAG | 60 |
| CGCCCCTGCC GCGGTGCCTG GCCTCCCCTC CCAGACTGCA GGGACAGCAC CCGGTAACTG | 120 |
| CGAGTGGAGC GGAGGACCCG AGCGGCTGAG GAGAGAGGAG GCGGCGGCTT AGCTGCTACG | 180 |
| GGGTCCGGCC GGCGCCCTCC CGAGGGGGGC TCAGGAGGAG GAAGGAGGAC CCGTGCGAGA | 240 |
| ATGCCTCTGC CCTGGAGCCT TGCGCTCCCG CTGCTGCTCT CCTGGGTGGC AGGTGGTTTC | 300 |
| GGGAACGCGG CCAGTGCAAG GCATCACGGG TTGTTAGCAT CGGCACGTCA GCCTGGGGTC | 360 |
| TGTCACTATG GAACTAAACT GGCCTGCTGC TAGGGCTGGA GAAGAAACAG CAAGGGAGTC | 420 |
| TGTGAAGCTA CATGCGAACC TGGATGTAAG TTTGGTGAGT GCGTGGGACC AAACAAATGC | 480 |
| AGATGCTTTC CAGGATACAC CGGGAAAACC TGCAGTCAAG ATGTGAATGA GTGTGGAATG | 540 |
| AAACCCCGGC CATGCCAACA CAGATGTGTG AATACACACG GAAGCTACAA GTGCTTTTGC | 600 |
| CTCAGTGGCC ACATGCTCAT GCCAGATGCT ACGTGTGTGA ACTCTAGGAC ATGTGCCATG | 660 |
| ATAAACTGTC AGTACAGCTG TGAAGACACA GAAGAAGGGC CACAGTGCCT GTGTCCATCC | 720 |
| TCAGGACTCC GCCTGGCCCC AAATGGAAGA GACTGTCTAG ATATTGATGA ATGTGCCTCT | 780 |
| GGTAAAGTCA TCTGTCCCTA CAATCGAAGA TGTGTGAACA CATTTGGAAG CTACTACTGC | 840 |
| AAATGTCACA TTGGTTTCGA ACTGCAATAT ATCAGTGGAC GATATGACTG TATAGATATA | 900 |
| AATGAATGTA CTATGGATAG CCATACGTGC AGCCACCATG CCAATTGCTT CAATACCCAA | 960 |
| GGGTCCTTCA AGTGTAAATG CAAGCAGGGA TATAAAGGCA ATGGACTTCG GTGGTCTGCT | 1020 |
| ATCCCTGAAA ATTCTGTGAA GGAAGTCCTC AGAGCACCTG GTACCATCAA AGACAGAATC | 1080 |
| AAGAAGTTGC TTGCTCACAA AAACAGCATG AAAAAGAAGG CAAAAATTAA AAATGTTACC | 1140 |
| CCAGAACCCA CCAGGACTCC TACCCCTAAG GTGAACTTGC AGCCCTTCAA CTATGAAGAG | 1200 |
| ATAGTTTCCA GAGGCGGGAA CTCTCATGGA GGTAAAAAAG GGAATGAAGA GAAAATGAAA | 1260 |
| GAGGGGCTTG AGGATGAGAA AAGAGAAGAG AAAGCCCTGA AGAATGACAT AGAGGAGCGA | 1320 |
| AGCCTGCGAG GAGATGTGTT TTTCCCTAAG GTGAATGAAG CAGGTGAATT CGGCCTGATT | 1380 |
| CTGGTCCAAA GGAAAGCGCT AACTfCCAAA CTGGAACATA AAGATTTAAA TATCTCGGTT | 1440 |
| GACTGCAGCT TCAATCATGG GATCTGTGAC TGGAAACAGG ATAGAGAAGA TGATTTTGAC | 1500 |
| TGGAATCCTG CTGATCGAGA TAATGCTATT GGCTTCTATA TGGCAGTTCC GGCCTTGGCA | 1560 |
| GGTCACAAGA AAGACATTGG CCGATTGAAA C1TCTCCTAC CTGACCTGCA ACCCCAAAGC | 1620 |
| AACTTCTGTT TGCTCGTTGA TTACCGGCTG GCCGGAGACA AAGTCGGGAA ACTTCGAGTG | 1680 |
| TTTGTGAAAA ACAGTAACAA TGCCCTGGCA TGGGAGAAGA CCACGAGTGA GGATGAAAAG | 1740 |
| TGGAAGACAG GGAAAATTCA GTfGTATCAA GGAACTGATG CTACCAAAAG CATCATTTTT | 1800 |
| GAAGCAGAAC GTGGCAAGGG CAAAACCGGC GAAATCGCAG TGGATGGCGT CTTGCTTGTT | 1860 |
| TCAGGCTTAT GTCCAGATAG CCTTTTATCT GTGGATGACT GAATGTTACT ATCTTTATAT | 1920 |
| TTGACTTTGT ATGTCAGTTC CCTGGTTTTT TTGATATTGC ATCATAGGAC CTCTGGCATT | 1980 |
| TTAGAATTAC TAGCTGAAAA ATTGTAATGT ACCAACAGAA ATATTATTGT AAGATGCCTT | 2040 |
| TCTTGTATAA GATATGCCAA TATTITGCTT AAATATCATA TCACTGTATC TTCTCAGTCA | 2100 |
| TTTCTGAATC TTTCCACATT ATATTATAAA ATATGGAAAT GTCAGTTTAT CTCCCCTCCT | 2160 |
| CAGTATATCT GATTTGTATA AGTAAGTTGA TGAGCTTCTC TCTACAACAT TTCTAGAAAA | 2220 |
| TAGAAAAAAA AGCACAGAGA AATGTTTAAC TGTTTGACTC TTATGATACT TCTTGGAAAC | 2280 |
| TATGACATCA AAGATAGACT TTTGCCTAAG TGGCTTAGCT GGGTCTTTCA TAGCCAAACT | 2340 |
| TGTATATTTA AATTCTTTGT AATAATAATA TCCAAATCAT CAAAAAAAAA AAAAAAAA | |

Seq ID NO: 102 Protein sequence:
Protein Accession #: NP_056322.2

| | |
|---|---|
| 1 11 21 31 41 51 | |
| \| \| \| \| \| \| | |
| MPLPWSLALP LLLSWVAGGF GNAASARHHG LLASARQPGV CBYGTKLACC YGWRRNSKGV | 60 |
| CEATCEPGCK EGECVGPNKC RCFPGYTGKT CSQDVNECGM KPRPCQHRCV NTHGSYKCFC | 120 |
| LSGHMLMPDA TCVNSRTCAM INCQYSCEDT EEGPQCLCPS SGLRLAPNGR DCLDIDECAS | 180 |
| GKVICPYNRR CVNTFGSYYC KCHIGFELQY ISGRYDCIDI NECTMDSHTC SHHANCFNTQ | 240 |
| GSFKCKCKQG YKGNGLRCSA IPENSVKEVL RAPGTIKDRI KKLLAHKNSM KKKAKIKNVT | 300 |
| PEPTRTPTPK VNLQPFNYEE IVSRGGNSHG GKKGNEEKMK EGLEDEKREE KALKNDIEER | 360 |
| SLRGDVFFPK VNEAGEFGLI LVQRKALTSK LEHKDLNISV DCSFNHGICD WKQDREDDFD | 420 |
| WNPADRDNAI GFYMAVPALA GHKKDIGRLK LLLPDLQPQS NFCLLFDYRL AGDKVGKLRV | 480 |
| FVKNSNNALA WEKTTSEDEK WKTGKIQLYQ GTDATKSIIF EAERGKGKTG EIAVDGVLLV | 540 |
| SGLCPDSLLS VDD | |

Seq ID NO: 103 DNA sequence
Nucleic Acid Accession #: NM_001565.1
Coding sequence: 67-363 (underlined sequences correspond to start and
stop codons)

| | |
|---|---|
| 1 11 21 31 41 51 | |
| \| \| \| \| \| \| | |
| GAGACATTCC TCAATTGCTT AGACATAYfC TGAGCCTACA GCAGAGGAAC CTCCAGTCTC | 60 |
| AGCACCATGA ATCAAACTGC GATTCTGATT TGCTGCCTTA TCTTTCTGAC TCTAAGTGGC | 120 |
| ATTCAAGGAG TACCTCTCTC TAGAACCGTA CGCTGTACCT GCATCAGCAT TAGTAATCAA | 180 |
| CCTGTTAATC CAAGGTCTTT AGAAAAACTT GAAATTATTC CTGCAAGCCA ATTTTGTCCA | 240 |
| CGTGTTGAGA TCATTGCTAC AATGAAAAAG AAGGGTGAGA AGAGATGTCT GAATCCAGAA | 300 |
| TCGAAGGCCA TCAAGAATTT ACTGAAAGCA GTTAGCAAGG AAATGTCTAA AAGATCTCCT | 360 |
| TAAAACCAGA GGGGAGCAAA ATCGATGCAG TGCTTCCAAG GATGGACCAC ACAGAGGCTG | 420 |
| CCTCTCCCAT CACTCCCCTA CATGGAGTAT ATGTCAAGCC ATAATTGTTC TTAGTTTGCA | 480 |
| GTTACACTAA AAGGTGACCA ATGATGGTCA CCAAATCAGC TGCTACTACT CCTGTAGGAA | 540 |
| GGTTAATGTT CATCATCCTA AGCTATTCAG TAATAACTCT ACCCTGGCAC TATAATGTAA | 600 |
| GCTCTACTGA GGTGCTATGT TCTTAGTGGA TGTTCTGACC CTGCTTCAAA TATTTCCCTC | 660 |
| ACCTTTCCCA TCTTCCAAGG GTACTAAGGA ATCTTTCTGC TTTGGGGITT ATCAGAATTC | 720 |
| TCAGAATCTC AAATAACTAA AATTTATGCA ATCAAATCTG CTTTTTAAAG AATGCTCTTT | 780 |
| ACTTCATGGA CTTCCACTGC CATCCTCCCA AGGGGCCCAA ATTCTTTCAG TGGCTACCTA | 840 |
| CATACAATTC CAAACACATA CAGGAAGGTA GAAATATCTG AAAATGTATG TGTAAGTATT | 900 |
| CTTATTTAAT GAAAGACTGT ACAAAGTATA AGTCTTAGAT GTATATATTT CCTATATCGT | 960 |
| TTTCAGTGTA CATGGAATAA CATGTAATTA AGTACTATGT ATCAATGAGT AACAGGAAAA | 1020 |
| TTTTAAAAAT ACAGATAGAT ATATGCTCTG CATGTTACAT AAGATAAATG TGCTGAATGG | 1080 |
| TTTTCAAATA AAAATGAGGT ACTCTCCTGG AAATATTAAG | |

Seq ID NO: 104 Protein sequence:
Protein Accession #: NP_001556.1

| | |
|---|---|
| 1 11 21 31 41 51 | |
| \| \| \| \| \| \| | |
| MNQTAILICC LWLTLSGIQ GVPLSRTVRC TCISISNQPV NPRSLEKLEI IPASQFCPRV | 60 |
| EIIATMKKKG EKRCLNPESK AIKNLLKAVS KEMSKRSP | |

Seq ID NO: 105 DNA sequence
Nucleic Acid Accession #: NM_015068.1
Coding sequence: 1170-2243 (underlined sequences correspond to start
and stop codons)

| | |
|---|---|
| 1 11 21 31 41 51 | |
| \| \| \| \| \| \| | |
| GTAACAACCG TCACCCTGGG TCCCGACTGC CCACCTCCTC CTCCTCCCCC TCCCCCCAAC | 60 |
| AACAACAACA ACAACAACTC CAAGCACACC GGCCATAAGA GTGCGTGTGT CCCCAACATG | 120 |
| ACCGAACGAA GAAGGGACGA GCTCTCTGAA GAGATCAACA ACTTAAGAGA GAAGGTCATG | 180 |
| AAGCAGTCGG AGGAGAACAA CAACCTGCAG AGCCAGGTGC AGAAGCTCAC AGAGGAGAAC | 240 |
| ACCACCCTTC GAGAGCAAGT GGAACCCACC CCTGAGGATG AGGATGATGA CATCGAGCTC | 300 |
| CGCGGTGCTG CAGCAGCTGC TGCCCCACCC CCTCCAATAG AGGAAGAGTG CCCAGAAGAC | 360 |
| CTCCCAGAGA AGTTCGATGG CAACCCAGAC ATGCTGGCTC CTTTCATGGC CCAGTGCCAG | 420 |
| ATCTTCATGG AAAAGAGCAC CAGGGATTTC TCAGTTGATC GTGTCCGTGT CTGCTTCGTG | 480 |
| ACAAGCATGA TGACCGGCCG TGCTGCCCGT TGGGCCTCAG CAAAGCTGGA GCGCTCCCAC | 540 |
| TACCTGATGC ACAACTACCC AGCTTTCATG ATGGAAATGA AGCATGTGGT TGAAGACCCT | 600 |
| CAGAGGCGAG AGGTTGCCAA ACGCAAGATC AGACGCCTGC GCCAAGGCAT GGGGTCTGTC | 660 |
| ATCGACTACT CCAATGCTTT CCAGATGATT GCCCAGGACC TGGATTGGAA CGAGCCTGCG | 720 |
| CTGATTGACC AGTACCACGA GGGCCTCAGC GACCACATTC AGGAGGAGCT CTCCCACCTC | 780 |
| GAGGTCGCCA AGTCGCTGTC TGCTCTGATT GGGCAGTGCA TTCACATTGA GAGAAGGCTG | 840 |
| GCCAGGGCTG CTGCAGCTCG CAAGCCACGC TCGCCACCCC GGGCGCTGGT GTTGCCTCAC | 900 |
| ATTGCAAGCC ACCACCAGGT AGATCCAACC GAGCCGGTGG GAGGTGCCCG CATGCGCCTG | 960 |
| ACGCAGGAAG AAAAAGAAAG ACGCAGAAAG CTGAACCTGT GCCTCTACTG TGGAACAGGA | 1020 |
| GGTCACTACG CTGACAATTG TCCTGCCAAG GCCTCAAAGT CTTCGCCGGC GGGAAACTCC | 1080 |
| CCGGCCCCGC TGTAGAGGGA CC1TCAGCGA CCGGGCCAGA AATAATAAGG TCCCCACAAG | 1140 |
| ATGATGCCTC ATCTCCACAC TTGCAAGTGA TGCTCCAGAT TCATCTTCCG GGCAGACACA | 1200 |
| CCCTGTTCGT CCGAGCCATG ATCGATTCTG GTGCTTCTGG CAACTTCATT GATCACGAAT | 1260 |
| ATGTTGCTCA AAATGGAATT CCTCTAAGAA TCAAGGACTG GCCAATACTT GTGGAAGCAA | 1320 |
| TTGATGGGCG CCCCATAGCA TCGGGCCCAG TTGTCCACGA AACTCACGAC CTGATAGTTG | 1380 |
| ACCTGGGAGA TCACCGAGAG GTGCTGTCAT TTGATGTGAC TCAGTCTCCA TTCTTCCCTG | 1440 |
| TCGTCCTAGG GGTTCGCTGG CTGAGCACAC ATGATCCCAA TATCACATGG AGCACTCGAT | 1500 |
| CTATCGTCTT TGATTCTGAA TACTGCCGCT ACCACTGCCG GATGTATTCT CCAATACCAC | 1560 |
| CATCGCTCCC ACCACCAGCA CCACAACCGC CACTCTATTA TCCAGTAGAT GGATACAGAG | 1620 |
| TTTACCAACC AGTGAGGTAT TACTATGTCC AGAATGTGTA CACTCCAGTA GATGAGCACG | 1680 |
| TCTACCCAGA TCACCGCCTG GTTGACCCTC ACATAGAAAT GATACCTGGA GCACACAGTA | 1740 |
| TTCCCAGTGG ACATGTGTAT TCACTGTCCG AACCTGAAAT GGCAGCTCTT CGAGATTTTG | 1800 |
| TGGCAAGAAA TGTAAAAGAT GGGCTAATTA CTCCAACGAT TGCACCTAAT GGAGCCCAAG | 1860 |
| TTCTCCAGGT GAAGAGGGGG TGGAAACTGC AAGTTTCTTA TGATTGCCGA GCTCCAAACA | 1920 |
| ATTTTACTAT CCAGAATCAG TATCCTCGCC TATCTATTCC AAATTTAGAA GACCAAGCAC | 1980 |
| ACCTGGCAAC GTACACTGAA TTCGTACCTC AAATACCTGG ATACCAAACA TACCCCACAT | 2040 |
| ATGCCGCGTA CCCGACCTAC CCAGTAGGAT TCGCCTGGTA CCCAGTGGGA CGAGACGGAC | 2100 |
| AAGGAAGATC ACTATATGTA CCTGTGATGA TCACTTGGAA TCCACACTGG TACCGCCAGC | 2160 |
| CTCCGGTACC ACAGTACCCG CCGCCACAGC CGCCGCCTCC ACCACCACCA CCGCCGCCGC | 2220 |
| CTCCATCTTA CAGTACCCTG TAAATACCTG TCATGTCCTT CAGGATCTCT GCCCTCAAAA | 2280 |
| TTTATTCCTG TTCAGCTTCT CAATCAGTGA CTGTGTGCTA AATTTTAGGC TACTGTATCT | 2340 |
| TCAGGCCACC TGAGGCACAT CCTCTCTGAA ACGGCTATGG AAGGTTAGGG CCACTCTGGA | 2400 |
| CTGGCACACA TCCTAAAGCA CCAAAAGACC TTCAACATTT TCTGAGAGCA ACAGAGTATT | 2460 |
| TGCCAATAAA TGATCTCTCA TTTTTCCACC TTGACTGCCA ATCTAACTAA AATAATTAAT | 2520 |
| AAGTTTACTT TCCAGCCAGT CCTGGAAGTC TGGGTTTTAC CTGCCAAAAC CTCCATCACC | 2580 |
| ATCTAAATTA TAGGCTGCCA AATTTGCTGT TTAACATTTA CAGAGAAGCT GATACAAACG | 2640 |
| CAGGAAATGC TGATTTCTTT ATGGAGGGGG AGACGAGGAG QAGGAGGACA TGACTTTTCT | 2700 |
| TGCGGTTTCG GTACCCTCTT TTTAAATCAC TGGAGGACTG AGGCCTTATT AAGGAAGCCA | 2760 |
| AAATTATCGG TGCAGTGTGG AAAGGCTTCC GTGATCCTCT CGCTGCACCC TTAGAAACTT | 2820 |
| CACCGTCTTC AAACTCCATT TCCATGGTTC TGTTAATTCT CAAGGAGCAG CAACTCGACT | 2880 |
| GGTTCTCCCA GGAGCAGGAA AAACCCTTGT GACATGAAAC ATCTCAGGCC TGAAAAGAAA | 2940 |
| GTGCTCTCTC AGATGGACTC TTGCATGTTA AGACTATGTC TTCACATCAT GGTGCAAATC | 3000 |
| ACATGTACCC AATGACTCCG GCTTTGACAC AACACCTTAC CATCATCATG CCATGATGGC | 3060 |
| TTCCACAAAG CATTAAACCT GGTAACCAGA GATTACTGGT GGCTCCAGCG TTGTTAGATG | 3120 |
| TTCATGAAAT GTGACCACCT CTCAATCACC TTTGAGGGCT AAAGAGTAGC ACATCAAAAG | 3180 |
| GACTCCAAAA TCCCATACCC AACTCTTAAG AGATTTGTCC TGGTACTTCA GAAAGAATTT | 3240 |
| TCATGAGTGT TCTTAATTGG CTGGAAAAGC ACCAGCTGAC GTTTTGGAAG AATCTATCCA | 3300 |
| TGTGTCTGCC TCCATATGCA TCTGGGCATT TCATCTTCAG TCCCCTCATT AGACTGTAGC | 3360 |
| ATTAGGATGT GTGGAGAGAG GAGAAATGAT TTAGCACCCA GATTCACACT CCTATGCCTG | 3420 |
| GAAGGGGGAC ATCTTTGAAG AAGAGGAATT AGGGCTGTGG ACACTGTCTT GAGGATGTGG | 3480 |
| ACTTCCTTAG TGAGCTCCAC ATTACTTGAT GGTAACCAGT TCAAAAGGAT CAGAATCCAC | 3540 |
| GTAATGAAAA AGGTCCCTCT AGAGGATGGA GCTGATGTGA AGCTGCCAAT GGATGAAAAG | 3600 |
| CCTCAGAAAG CAACTCAAAG GACTCAAAGC AACGGACAAC ACAAGAGTTG TCTTCAGCCC | 3660 |
| AGTGACACCT CTGATGTCCC CTGGAAGCTT TGTGCTAACC TGGGACTGCC TGACTTCCTT | 3720 |
| TAGCCTGGTC CCTTGCTACT ACCTTGAACT GTTTTATCTA ACCTCTCTTT TTCTGTTTAA | 3780 |
| TTCTTTGCTA CTGCCATTGA CCCTGCTGCA GGATITGTGT CATTTTCCTG CCTGGTTGCT | 3840 |
| GAGACTCCAT TTTGCTGCCA CACACAGAGA TGTAAGAGGC AGGCTTTAAT TGCCAAAGCA | 3900 |
| CAGTTTGAGC AGTAGAAAAC AACATGGTGT ATATCTCAAA TTGCCTGACA TGAAGAGGAG | 3960 |
| TCTAACGGTG AAGTTTCACT TTTCATCAGC ATCATCTTTC ACATGTTCAT TATCATCCGC | 4020 |
| TCTAACGGTG AAGTTTCACT TTTCATCAGC ATCATCTTTC ACATGTTCAT TATCATCCGC | 4080 |
| GTGGTGACTA GGCTTTCAAA AACTTCCATT GAATTACAAA GCACTATCCA GTTCTTATTG | 4140 |
| TTAAACTAAG TAAAAATGAT AAGTAACATA GTGTAAAATA TTCCTTTACT GTGAACTTCT | 4200 |
| TACAATGCTG TGAATGAGAG GCTCCTCAGA ACTGGAGCAT TTGTATAATA ATTCATCCTG | 4260 |
| TTCATCTTCA ATTTTAACAT CATATATAAT TTCAATTCTA TCAATTGGGC CTTTAAAAAT | 4320 |
| CATATAAAAG GATATAAAAT TTGAAAAGAG AAACCTAATT GGCTATTTAA TCCAAAACAA | 4380 |
| CTTTITITTT TCCTTCAATG GAATCAGAAA GCTTGTCAAT CACTCATGTG TFTTAGAGTA | 4440 |
| ATTACTTTTA AAATGGTGCA TTTGTGCTTC TGAACTATTT TGAAGAGTCA CTTCTGTTTA | 4500 |
| CCTCAAGTAT CAATTCATCC TCCATACATT TGAATTCAAG TTGTTTTTTG TCAAATTTAC | 4560 |
| AGTTGTCAAT TGATCTTCAA GCTGCAGGGT GCCTAGAAAT GGGCCGTTGT CTGTAGCCCT | 4620 |
| GGCATGTGCA CACGGACATT TGCCACCACT GCAAGCAAAA GTCTGGAGAA GTTCACCAAC | 4680 |
| GACAAGAACG ATTAGGGAAA ATATGCTGCT GTGGGTTAAC AACTCAGAAA GTCCCTGATC | 4740 |
| CACATTTGGC TGTTTACTAA AGCTTGTGAT TAACTTTTTG GCAGTGTGTA CTATGCTCTA | 4800 |
| TTGCTATATA TGCTATCTAT AAATGTAGAT GTTAAGGATA AGTAATTCTA AATTTATTAT | 4860 |
| TCTATAGTTT TGAAGTTTGG TTAAGTTTCC TTTCACTCAA TTGATTTATT TTGTTGTTAA | 4920 |
| TCAAATTTAT GTTAATTGGA TCCTTTAAAT TTTGTTTGGC ATTTTCCAAC AAAAATGGCT | 4980 |
| TTATTCATAA GAAAGGAAAA AAATCAATGG AATTTGATAT CTAAAGAAGT TAGAAAGGGA | 5040 |
| GCAAAATAAA AAACATAAAG GAGATAGATG AATTAGTAAG CAAATCAGTA GTCGAGTTTT | 5100 |
| TCAAACTGGC AAAATTAATT AATTGACTTT TAGCCCAAAT TTACATTGTT AATTAAATCA | 5160 |
| AGAAGGAAGA AGATCTAAGA GCTCCCATTG ATAGGCAAGC CTAGAGAGAA CTAGCTAAAT | 5220 |
| YfATCATGCT AGGATATTGA AACACAGAAA GTTTACATAC ATTTATGAAG GGTCAATTTA | 5280 |
| GTTTGGACAG TGAGGTATTT GTCTTAGTGG AAAAAAGGAG AATTAGTCTG ATCAAATCGT | 5340 |
| GAAGTAATAC AGTGAACTTG CAGGTGCACA AAATAAGAGG GCCACATCTA TATGGTGCAG | 5400 |
| TCTGGAATTC TGTTTAAGTT TGTAGGTACC TCTTGGACTT CTGAATTGAT CCAGTTGTCA | 5460 |
| TCCACCACAG ACATCTCACA TCAGATACAG ACAGTTCCAA GATTGACAAC AGAGAACAAC | 5520 |
| CTGCTGGAAA GACCTGGGCA GAAATGGAGA GCCCTGCGGG AACCATGCTA CATTTTCATC | 5580 |
| TAAAGAGAGA ATGCACATCT GATGAGACTG AAAGTTCTTT GTTGTTTTAG ATTGTAGAAT | 5640 |
| GGTATTGAAT TGGTCTGTGG AAAATTGCAT TGCTTTTATT TCTTTGTGTA ATCAAGTTTA | 5700 |
| AGTAATAGGG GATATATAAT CATAAGCATT TTAGGGTGGG AGGGACTATT AAGTAATTTT | 5760 |
| AAGTGGGTGG GGTTATTTAG AATGTTAGAA TAATATTATG TATTAGATAT CGCTATAAGT | 5820 |
| GGACATGCGT ACTTACTTGT AACCCTTTAC CCTATAATTG CTATCCTTAA AGATTTCAAA | 5880 |
| TAAACTCGGA GGGAACTGCA GGGAGACCAA CTTATTTAGA GCGAATTGGA CATGGATAAA | 5940 |
| AACCCCAGTG GGAGAAAGTT CAAAGGTGAT TAGATTAATA ATTTAATAGA GGATGAGTGA | 6000 |
| CCTCTGATAA ATTACTGCTA GAATGAACTT GTCAATGATG GATGGTAAAT TTTCATGGAA | 6060 |
| GTTATAAAAG TGATAAATAA AAACCCTTGC TTTTACCCCT GTCAGTAGCC CTCCTCCTAC | 6120 |
| CACTGAACCC CATTGCCCCT ACCCCTCCTT CTAACTTTAT TGCTGTATTC TCTTCACTCT | 6180 |
| ATATTTCTCT CTATTTGCTA ATATTGCATT GCTGTTACAA TAAAAATTCA ATAAAGATTT | 6240 |
| AGTGGTTAAG TGC | |

Seq ID NO: 106 Protein sequence:
Protein Accession #: NP_055883.1

| | |
|---|---|
| 1 11 21 31 41 51 | |
| \| \| \| \| \| \| | |
| MTERRRDELS EEINNLREKV MKQSEENNNL QSQVQKLTEE NTTLREQVEP TPEDEDDDIE | 60 |
| LRGAAAAAAP PPPIEEECPE DLPEKFDGNP DMLAPFMAQC QIFMEKSTRD FSVDRVRVCF | 120 |
| VTSMMTGRAA RWASAKLERS BYLMHNYPAF MMEMKHVFED PQRRBVAKRK IRRLRQGMGS | 180 |
| VIDYSNAFGM IAGDLDWNEP ALIDGYHEGL SDHIGEELSH LEVAKSLSAL IGQCIHIERR | 240 |
| LARAAAARKP RSPPRALVLP HIASHHQVDP TEPVGGARMR LTQEEKERRR IINLCLYCGT | 300 |
| GGHYADNCPA KASKSSPAGN SPAPL | |

Seq ID NO: 107 DNA sequence
Nucleic Acid Accession #: NM_003679.1
Coding sequence: 47-1507 (underlined sequences correspond to start and
stop codons)

| | |
|---|---|
| 1 11 21 31 41 51 | |
| \| \| \| \| \| \| | |
| GGCACGAGCA GAAGCAACAA TAATTGTGAA AAATACTTCA GCAGTTATGG ACTCATCTGT | 60 |
| CATTCAAAGG AAAAAAGTAG CTGTCATTGG TGGTGGCTTG GTTGGCTCAT TACAAGCATG | 120 |
| CTTTCTTGCA AAGAGGAATT TCCAGATTGA TGTATATGAA GCTAGGGAAG ATACTCGAGT | 180 |
| GGCTACCTTC ACACGTGGAA GAAGCATTAA CTTAGCCCTT TCTCATAGAG GACGACAAGC | 240 |
| CTTGAAAGCT GTTGGCCTGG AAGATCAGAT TGTATCCCAA GGTATTCCCA TGAGAGCAAG | 300 |
| AATGATCCAC TCTCTTTCAT GAAAAAAGTC TGCAATTCCC TATGGGACAA AGTCTCAGTA | 360 |
| TATTCTTTCT GTAAGCAGAG AAAATCTAAA CAAGGATCTA TTGACTGCTG CTGAGAAATA | 420 |
| CCCCAATGTG AAAATGCACT TTAACCACAG GCTTTTGAAA TGTAATCCAG AGGAAGGAAT | 480 |
| GATCACAGTG CTTGGATCTG ACAAAGTTCC CAAAGATGTC ACTTGTGACC TCATTGTAGG | 540 |
| ATGTGATGGA GCCTATTCAA CTGTCAGATC TCACCTGATG AAGAAACCTC GCTTTGATTA | 600 |
| CAGTCAGCAG TACATTCCTC ATGGGTACAT GGAGTTGACT ATTCCACCTA AGAACGTAGA | 660 |
| TTATGCCATG GAACCTAATT ATCTGCATAT TTGGCCTAGA AATACCTTTA TGATGATTGC | 720 |
| ACTTCCTAAC ATGAACAAAT CATTCACATG TACTTTGTTC ATGCCCTTTG AAGAGTTfGA | 780 |
| AAAACTTCTA ACCAGTAATG ATGTGGTAGA TTTCTTCCAG AAATACTTTC CGGATGCCAT | 840 |
| CCCTCTAATT GGAGAGAAAC TCCTAGTGCA AGATTTCTTC CTGTTGCCTG CCCAGCCCAT | 900 |
| GATATCTGTA AAGTGCTCTT CATTTCACTT TAAATCTCAC TGTGTACTGC TGGGAGATGC | 960 |
| AGCTCATGCT ATAGTGCCGT TTTTTGGGCA AGGAATGAAT GCGGGCTTTG AAGACTGCTT | 1020 |
| GGTATTTGAT GAGTTAATGG ATAAATTCAG TAACGACCTT AGTTTGTGTC TTCCTGTGTT | 1080 |
| CTCAAGATTG AGAATCCCAG ATGATCACGC GAYFTCAGAC CTATCCATGT ACAATTACAT | 1140 |
| AGAGATGCGA GCACATGTCA ACTCAAGCTG GTTCATTTTT CAGAAGAACA TGGAGAGATT | 1200 |
| TCTTCATGCG ATTATGCCAT CGACCTTTAT CCCTCTCTAT ACAATGGTCA CTTTTTCCAG | 1260 |
| AATAAGATAC CATGAGGCTG TGCAGCGTTG GCATTGGCAA AAAAAGGTGA TAAACAAAGG | 1320 |
| ACTCTTTTTC TTGGGATCAC TGATAGCCAT CAGCAGTACC TACCTACTTA TACACTACAT | 1380 |
| GTCACCACGA TCTTTCCTCT GCTTGAGAAG ACCATGGAAC TGGATAGCTC ACTITCCGGA | 1440 |
| TACAACATGT TTCCCCGCAA AGGCCGTGGA CTCCCTAGAA CAAATTTCCA ATCTCATTAG | 1500 |
| CAGGTGATAG AAAGGTTTTG TGGTAGCAAA TGCATGATTT CTCTGTGACC AAAATTAAGC | 1560 |
| ATGAAAAAAA TGTTTCCATT GCCATATTTG ATTCACTAGT GGAAGATAGT GTTCTGCTTA | 1620 |
| TAATTAAACT GAATGTAGAG TATCTCTGTA TGTTAATTGC AATTACTGGT TGGGGGGTGC | 1680 |
| ATTTTAAAAG ATGAAACATG CAGCTTCCCT ACATTACACA CACTCAGGTT GAGTCATTCT | 1740 |
| AACTATAAAA GTGCAATGAC TAAGATCCTT CACTTCTCTG AAAGTAAGGC CCTAGATGCC | 1800 |
| TCAGGGAAGA CAGTAATCAT GCCTTTTCTT TAAAAGACAC AATAGGACTC GCAACAGCAT | 1860 |
| TGACTCAACA CCTAGGACTA AAAATCACAA CTTAACTAGC ATGTTAACTG CACTTTTCAT | 1920 |
| TACGTGAATG GAACTTACCT AACCACAGGG CTCAGACTTA CTAGATAAAA CCAGAAATGG | 1980 |
| AAATAAGGAA TTCAGGGGAG TTCCAGAGAC TTACAAAATG AACTCATTTT ATTTTCCCAC | 2040 |
| CTTCAAATAT AAGTATTATC ATCTATCTGT TTATCGTCTA TCTATCTATC ATCTATCTAT | 2100 |
| CTATCTATCA TCTATCTATC TATCTATCTA TCTATCTATC TATCTATCTA TCTATCTATC | 2160 |
| TCTATTTATT TATGTATITA GAGATCAGGT CTCACTCTGT TGACCAGGCT GGAGTGCAGT | 2220 |
| GGTGAGATCT GGGTTCACTG CAACCTCTGC CTCCTGGGCT CAAGCAATCC TCCCACTTCA | 2280 |
| GCCTCCCAAA TAGCTGGGGC TACCATGGTA TTTTTCAGTA GAGACCGGGT CTTGCCATGC | 2340 |
| TGCCCAGGCC AGTCTCAAAC TCCTGGCCTC ATGTGATCTG CCCACCTCAG CCTCCCAAAG | 2400 |
| TACAGGGATT AGAGTTGTGA GCCACCGCTG CCAGCCCAGA GTTACCCTCT AAAGATAAGA | 2460 |
| AAAAGGCTAT TAATATCATA CTAAGTGAAG GACAGGAAAG GGTTTTATTC ATAAATTAAA | 2520 |
| TGTCTACATG TGCCAGAATG GAAAGGAAAC AAGGGGAGAC AACTTTTATA GAAATACAAA | 2580 |
| GCCAYTACTT TATTCAATTT CAGACCCTCA GAAGCAATTT ACTAATTTAT TCTTCGACTA | 2640 |
| CATACTGCAG CAGAACCAGC AATACACTTG ATTTTTAAAA GCACATTTAG TGAAATGTTT | 2700 |
| TCTTTGGTTC ATCCTTCTTT AACAGGCTGC TGAGTCACTC AGAAATCCTT CAAACATGAT | 2760 |
| TAATTATGAA GATGAAACAC TAGAGTCATA TAAGAAATAA AAATTGGGCA ATAAAATAAA | 2820 |
| ATGATFCAGT GTTTCTTTTC TATATTGTCA ATGAAAACCT TGAGTTCTAA TAATCCATGT | 2880 |
| TCAGTTTGTA GGGAAAGAAA AAATAATTTT TCCTTCTACC CACTTTAGGT TCCTTGGCTG | 2940 |
| GGGCCCCTAT AACAAAAGAC AGATTGACAA GAGAAAAACA AACATAAATT TATTAGCGGG | 3000 |
| TATATGTAAT ATATATGTGG GAAATACAGG GGAATGAGCA AATCTCAAAG AGCTGGCGTC | 3060 |
| TTAGAACTCC CTGGCTTATA TAGCATCGAC AAAGAACAGT AAATTTTTAG AGAAACAACA | 3120 |
| AAACAAAGAA AAAGAGCTTT GAGTCTGTAG GGGCAGCAAT TTGGGGGAAG CAAATATATG | 3180 |
| GGAGTTTGCC TTGTAGATTC CTCTGGTGGT GGTCTCCAGG CTGACAAGGA TTCAAAGTTG | 3240 |
| TCTCTGAAAC TCCTCTTTGT CATACTGCAC ATATAAAACG TCTTTTGTTT CCAACAAGAG | 3300 |
| GATTTCTTTT TCATTCTAGA ATTATCTCCT TGATAACCTG ATCAGATATA GGACATGACA | 3360 |
| CTTAATAGAG TCCAACAGTA CAAAAAAAAT TCAGTATGTT CTAGCTACTT CACACATGTG | 3420 |
| TACGCGACAG TTATTTTTAC AGTAAGGTAT TTTCGAGAAA AATGCATTAC GTGTTTTGGA | 3480 |
| AAATAGAGTA ATTTAAAAAA TATATTTGAA ATCTAAAACT CCAACACATT AGAAGATGAT | 3540 |
| GATGTTAGAT GCCCATCGTG TGCCACAAGT GGTTTTTTCA TTATGTAAAG CACCCGTTGA | 3600 |
| ATTAAAAGAA TTTGTTTTTG TTCAACCTCT TCCTGAGGCC CAAGAGCATA TGGGCAATTC | 3660 |
| GGATTTCCTG CTGGACCACA AGGTTCTGTT GATATTACAT AGAAACGGGT ATTCCAGACA | 3720 |
| CTTCTTATGA TGAAAGTCCA AAAGTGGCAT CCAATTTAAG GCCCCATCTT TCGTTGCCAT | 3780 |
| TCTTCATTCC TACAAAGGAC GAACTTGGAT TACATCAACT TTGGACCCAT TGGTTTTGTC | 3840 |
| GCTGTCGTCA ACTGACAGTG ATTCACCACT GGTGATGATA AAAATGATGG AAGAAGAGTT | 3900 |
| GAAAGTCACT TTTTTCTTTG GCCTGTCCCC ATCTTTCTGT GACATCACAA TGGGTCTGAT | 3960 |
| CTGCATTTCA CTTCCAGCTG CTGGTAGGTC TTTAGCAGGC CTCTGGCACC TCAGCAGTCG | 4020 |
| GAGGCACAGA AGCTGCAAAA GGGATCTTCG AAACTGGGCA GAGAAAAAAT AAAGTGGAAT | 4080 |
| ATTAAGTAAA AGTTGGGCAC TAATCTGGAT TAACATTCGA GGAAATCAGT TGAGCTGATT | 4140 |
| TAAGTTGTTT TTTGTTTGTT AGCAGGTGTG GATGTGGGGT TATGTGGTCA TGCTCAGATC | 4200 |
| TACCTAAATC ACCCCAGAGC TTTATGTCTT TTATTCATTC TAAATCTTAT TAACCGGAAT | 4260 |
| ATGTAGGACC ATTTCAATAC CTTGTAATCC TCCAAGCTTC AATGTGCACA CACTTTCTAT | 4320 |
| GAGGGCAGGT ACAACTATTA AGAGATTTTG AACATTAAGT TAGTCCACAA ATATICAGTG | 4380 |
| GGCATCTACT AGGTGACAGC CACTGTGCTA TAATTAGAGA CTTTTTACTA TAAGCATCAA | 4440 |
| AAACAGATAA GGCTCTTCCT GGCAGAGTTT ACAGCCTGGT GTACTTGCTA ATGTCTCTTT | 4500 |
| AATTAGGTGA AGAATTTTTT TTTTCTATCG AAATTACTAA TCAG1TGGGG AAAAAAATAC | 4560 |
| TATAGCAGAC AGCACTAATG TCATCAACAA ACATTGTTCT TCTCCGTGTC CTGGGTACAA | 4620 |
| CATCGAATAA TATTTCTTGG CCTCCTTTCC GCTTCTCCTC TCTGCTGTTC CTCTCTACAA | 4680 |
| GAACCTGGGA GGCCAACGCC TAAAGATCAT AATATCACAA TGGAAGGAAC CTAGATTCCT | 4140 |
| AAATGACTGC ATAGGACAGA TCCCATCTCC TCCACCCAAT ACATTATTAG ACTGAACTGT | 4800 |
| GACCTGAAAT GAGCAATAAA CTCTGTATTA ATTCACTGAA ATGTTGGGGT TGCTTGTTAT | 4860 |
| AGTAGTCGGT CCATCATGAC CAGTAAAACA TAAATCAAAA GTTAATGTAA TTGTTATCCC | 4920 |
| ATTATTTAGA GCGAAATAAA TGTTGAATAT ATGGACTTTC TCAGATTAGG AAATACCAAT | 4980 |
| TAAAAATATA ATAAATAGCT | |

Seq ID NO: 108 Protein sequence:
Protein Accession #: NP_003670.1

| | |
|---|---|
| 1 11 21 31 41 51 | |
| \| \| \| \| \| \| | |
| MDSSVIQRKK VAVIGGGLVG SLQACFLAKR NFQIDVYEAR EDTRVATFTR GRSINLALSH | 60 |
| RGRQALKAVG LEDQIVSQGI PMRARMIHSL SGKKSAIPYG TKSQYILSVS RENLNKDLLT | 120 |
| AAEKYPNVKM HFNHRLLKCN PEEGMITVLG SDKVPKDVTC DLIVGCDGAY STVRSHLMKK | 180 |
| PRFDYSQQYI PHGYMELTIP PKNGDYAMEP NYLHIWPRNT FMMIALPNMN KSFTCTLFMP | 240 |
| FEEFEKLLTS NDVVDFFQKY FPDAIPLIGE KLLVQDFFLL PAQPMISVKC SSFHFKSHCV | 300 |
| LLGDAAHAIV PFFGQGMNAG FEDCLVFDEL MDKFSNDLSL CLPVFSRLRI PDDHAISDLS | 360 |
| MYNYIEMRAH VNSSWFIFQK NMERFLHAIM PSTFIPLYTM VTFSRIRYHE AVQRWHWQKK | 420 |
| VINKGLFFLG SLIAISSTYL LIHYMSPRSF LCLRRPWNWI AHFRNTTCFP AKAVDSLEQI | 480 |
| SNLISR | |

Seq ID NO: 109 DNA sequence
Nucleic Acid Accession #: NM_006115.1
Coding sequence: 236-1765 (underlined sequences correspond to start
and stop condons)

| | |
|---|---|
| 1 11 21 31 41 51 | |
| \| \| \| \| \| \| | |
| GCTTCAGGGT ACAGCTCCCC CGCAGCCAGA AGCCGGGCCT GCAGCCCCTC AGCACCGCTC | 60 |
| CGGGACACCC CACCCGCTTC CCAGGCGTGA CCTGTCAACA GCAACTTCGC GGTGTGGTGA | 120 |
| ACTCTCTGAG GAAAAACCAT TTTGATTATT ACTCTCAGAC GTGCGTGGCA ACAAGTGACT | 180 |
| GAGACCTAGA AATCCAAGCG TTGGAGGTCC TGAGGCCAGC CTAAGTCGCT TCAAAATGGA | 240 |
| ACGAAGGCGT TTGTGGGGTT CCATTCAGAG CCGATACATC AGCATGAGTG TGTGGACAAG | 300 |
| CGCACGGAGA CTTGTGGAGC TGGCAGGGCA GAGCCTGCTG AAGGATGAGG CCCTGGCCAT | 360 |
| TGCCGCCCTG GAGTTGCTGC CCAGGGAGCT CTTCCCGCCA CTCTTCATGG CAGCCTTTGA | 420 |
| CGGGAGACAC AGCCAGACCC TGAAGGCAAT GGTGCAGGCC TGGCCCTTCA CCTGCCTCCC | 480 |
| TCTGGGAGTG CTGATGAAGG GACAACATCT TCACCTGGAG ACCTTCAAAG CTGTGCTTGA | 540 |
| TGGACTTGAT GTGCTCCTTG CCCAGGAGGT TCGCCCCAGG AGGTGGAAAC TTCAAGTGCT | 600 |
| GGATTTACGG AAGAACTCTC ATCAGGACTT CTGGACTGTA TGGTCTGGAA ACAGGGCCAG | 660 |
| TCTGTACTCA TTTCCAGAGC CAGAAGCAGC TCAGCCCATG ACAAAGAAGC GAAAAGTAGA | 720 |
| TGGTTTGAGC ACAGAGGCAG AGCAGCCCTT CATTCCAGTA GAGGTGCTCG TAGACCTGTT | 780 |
| CCTCAAGGAA GGTGCCTGTG ATGAATTGTT CTCCTACCTC ATTGAGAAAG TGAAGCGAAA | 840 |
| GAAAAATGTA CTACGCCTGT GCTGTAAGAA GCTGAAGATT TTTGCAATGC CCATGCAGGA | 900 |
| TATCAAGATG ATCCTGAAAA TGGTGCAGCT GGACTCTATT GAAGATTTGG AAGTGACTTG | 960 |
| TACCTGGAAG CTACCCACCT TGGCGAAATT TTCTCCTTAC CTGGGCCAGA TGATTAATCT | 1020 |
| GCGTAGACTC CTCCTCTCCC ACATCCATGC ATCTTCCTAC ATTTCCCCGG AGAAGGAAGA | 1080 |
| GCAGTATATC GCCCAGTTCA CCTCTCAGTT CCTCAGTCTG CAGTGCCTGC AGGCTCTCTA | 1140 |
| TGTGGACTCT TTATTTTTCC TTAGAGGCCG CCTGGATCAG TTGCTCAGGC ACGTGATGAA | 1200 |
| CCCCTTGGAA ACCCTCTCAA TAACTAACTG CCGGCTTTCG GAAGGGGATG TGATGCATCT | 1260 |
| GTCCCAGAGT CCCAGCGTCA GTCAGCTAAG TGTCCTGAGT CTAAGTGGGG TCATGCTGAC | 1320 |
| CGATGTAAGT CCCGAGCCCC TCCAAGCTCT GCTGGAGAGA GCCTCTGCCA CCCTCCAGGA | 1380 |
| CCTGGTCTTT GATGAGTGTG GGATCACGGA TGATCAGCTC CTTGCCCTCC TGCCTTCCCT | 1440 |
| GAGCCACTGC TCCCAGCTTA CAACCTTAAG CTTCTACGGG AATTCCATCT CCATATCTGC | 1500 |
| CTTGCAGAGT CTCCTGCAGC ACCTCATCGG GCTGAGCAAT CTGACCCACG TGCTGTATCC | 1560 |
| TGTCCCCCTG GAGAGTTATG AGGACATCCA TGGTACCCTC CACCTGGAGA GGCTTGCCTA | 1620 |
| TCTGCATGCC AGGCTCAGGG AGTTGCTGTG TGAGTTGGGG CGGCCCAGCA TGGTCTGGCT | 1680 |
| TAGTGCCAAC CCCTGTCCTC ACTGTGGGGA CAGAACCTTC TATGACCCGG AGCCCATCCT | 1740 |
| GTGCCCCTGT TTCATGCCTA ACTAGCTGGG TGCACATATC AAATGCTTCA TTCTGCATAC | 1800 |
| TTGGACACTA AAGCCAGGAT GTGCATGCAT CTTGAAGCAA CAAAGCAGCC ACAGTTTCAG | 1860 |
| ACAAATGTTC AGTGTGAGTG AGGAAAACAT GTTCAGTGAG GAAAAAACAT TCAGACAAAT | 1920 |
| GTTCAGTGAG GAAAAAAAGG GGAAGTTGGG GATAGGCAGA TGTTGACTTG AGGAGTTAAT | 1980 |
| GTGATCTTTG GGGAGATACA TCTTATAGAG TTAGAAATAG AATCTGAATT TCTAAAGGGA | 2040 |
| GATTCTGGCT TGGGAAGTAC ATGTAGGAGT TAATCCCTGT GTAGACTGTT GTAAAGAAAC | 2100 |
| TGTTGAAAAT AAAGAGAAGC AATGTGAAGC AAAAAAAAAA AAAAAAAA | |

Seq ID NO: 110 Protein sequence:
Protein Accession #: NP 006106.1

| | |
|---|---|
| 1 11 21 31 41 51 | |
| \| \| \| \| \| \| | |
| MERRRLWGSI QSRYISMSVW TSPRRLVELA GQSLLKDEAL AIAALELLPR ELFPPLFMAA | 60 |
| FDGRHSQTLK AMVQAWPFTC LPLGVLMKGQ HLHLETFKAV LDGLDVLLAQ EVRPRRWKLQ | 120 |
| VLDLRKNSHQ DFWTVWSGNR ASLYSEPEPE AAQPMTKKRK VDGLSTEAEQ PFIPVEVLVD | 180 |
| LFLKEGACDE LFSYLIEKVK RKKNVLRLCC KKLKIFAMPM QDIKMILKMV QLDSIEDLEV | 240 |
| TCTWKLPTLA KFSPYLGQMI NLRRLLLSHI HASSYISPEK EEQYIAQFTS QFLSLQCLQA | 300 |
| LYVDSLFFLR GRLDQLLRHV MNPLETLSIT NCRLSEGDVM HLSQSPSVSQ LSVLSLSGVM | 360 |
| LTDVSPEPLQ ALLERASATL QDLVFDECGI TDDQLLALLP SLSHCSQLTT LSFYGNSISI | 420 |
| SALQSLLQHL IHLSNLTHVL YPVPLESYED IHGTLHLERL AYLHARLREL LCELGRPSMV | 480 |
| WLSANPCPHC GDRTFYDPEP ILCPCFMPN | |

Seq ID NO: 111 DNA sequence
Nucleic Acid Accession #: NM_003815
Coding sequence: 8-2452 (underlined sequences correspond to start and
stop codons)

| | |
|---|---|
| 1 11 21 31 41 51 | |
| \| \| \| \| \| \| | |
| CGCTGCCATG CGGCTGGCGC TGCTCTGGGC CCTGGGGCTC CTGGGCGCGG GCAGCCCTCT | 60 |
| GCCTTCCTGG CCGCTCCCAA ATATAGGTGG CACTGAGGAG CAGCAGGCAG AGTCAGAGAA | 120 |
| GGCCCCGAGG GAGCCCTTGG AGCCCCAGGT CCTTCAGGAC GATCTCCCAA TTAGCCTCAA | 180 |
| AAAGGTGCTT CAGACCAGTC TGCCTGAGCC CCTGAGGATC AAGTTGGAGC TGGACGGTGA | 240 |
| CAGTCATATC CTGGAGCTGC TACAGAATAG GGAGTTGGTC CCAGGCCGCC CAACCCTGGT | 300 |
| GTGGTACCAG CCCGATGGCA CTCGGGTGGT CAGTGAGGGA CACACTTTGG AGAACTGCTG | 360 |
| CTACCAGGGA AGAGTGCGGG GATATGCAGG CTCCTGGGTG TCCATCTGCA CCTGCTCTGG | 420 |
| GCTCAGAGGC TTGGTGGTCC TGACCCCAGA GAGAAGCTAT ACCCTGGAGC AGGGGCCTGG | 480 |
| GGACCTTCAG GGTCCTCCCA TTATTTCGCG AATCCAAGAT CTCCACCTGC CAGGCCACAC | 540 |
| CTGTGCCCTG AGCTGGCGGG AATCTGTACA CACTCAGACG CCACCAGAGC ACCCCCTGGG | 600 |
| ACAGCGCCAC ATTCGCCGGA GGCGGGATGT GGTAACAGAG ACCAAGACTG TGGAGTTGGT | 660 |
| GATTGTGGCT GATCACTCGG AGGCCCAGAA ATACCGGGAC TCCCAGCACC TGCTAAACCG | 720 |
| CACACTGGAA GTGGCCCTCT TGCTGGACAC ATTCTTCCGG CCCCTGAATG TACGAGTGGC | 780 |
| ACTAGTGGGC CTGGAGGCCT GGACCCAGCG TGACCTGGTG GAGATCAGCC CAAACCCAGC | 840 |
| TGTCACCCTC GAAAACTTCC TCCACTGGCG CAGGGCACAT TTGCTGCCTC GATTGCCCCA | 900 |
| TGACAGTGCC CAGCTGGTGA CTGGTACTTC ATTCTCTGGG CCTACGGTGG GCATGGCCAT | 960 |
| TCAGAACTCC ATCTGTTCTC CTGACTTCTC AGGAGGTGTG AACATGGACC ACTCCACCAG | 1020 |
| CATCCTGGGA GTCGCCTCCT CCATAGCCCA TGAGTTGGGC CACAGCCTGG GCCTGGACCA | 1080 |
| TGATTTGCCT GGGAATAGCT GCCCCTGTCC AGGTCCAGCC CCAGCCAAGA CCTGCATCAT | 1140 |
| GGAGGCCTCC ACAGACTTCC TACCAGGCCT GAACTTCAGC AACTGCAGCC GACGGGCCCT | 1200 |
| GGAGAAAGCC CTCCTGGATG GAATGGGCAG CTGCCTCTTC GAACGGCTGC CTAGCCTACC | 1260 |
| CCCTATGGCT GCTTTCTGCG GAAATATGTT TGTGGAGCCG GGCGAGCAGT GTGACTGTGG | 1320 |
| CTTCCTGGAT GACTGCGTCG ATCCCTGCTG TGATTCTTTG ACCTGCCAGC TGAGGCCAGG | 1380 |
| TGCACAGTGT CCATCTGACG GACCCTGTTG TCAAAATTGC CAGCTGCGCC CGTCTGGCTG | 1440 |
| GCAGTGTCGT CCTACCAGAG GGGATTGTGA CTTGCCTGAA TTCTGCCCAG GAGACAGCTC | 1500 |
| CCAGTGTCCC CCTGATGTCA GCCTAGGGGA TGGCGAGCCC TGCGCTGGCG GGCAAGCTGT | 1560 |
| GTGCATGCAC GGGCGTTGTG CCTCCTATGC CCAGCAGTGC CAGTCACTTT GGGGACCTGG | 1620 |
| AGCCCAGCCC GCTGCGCCAC TTTGCCTCCA GACAGCTAAT ACTCGGGGAA ATGCTTTTGG | 1680 |
| GAGCTGTGGG CGCAACCCCA GTGGCAGTTA TGTGTCCTGC ACCCCTAGAG ATGCCATTTG | 1740 |
| TGGGCAGCTC CAGTGCCAGA CAGGTAGGAC CCAGCCTCTG CTGGGCTCCA TCCGGGATCT | 1800 |
| ACTCTGGGAG ACAATAGATG TGAATGGGAC TGAGCTGAAC TGCAGCTGGG TGCACCTGGA | 1860 |
| CCTGGGCAGT GATGTGGCCC AGCCCCTCCT GACTCTGCCT GGCACAGCCT GTGGCCCTGG | 1920 |
| CCTGGTGTGT ATAGACCATC GATGCCAGCG TGTGGATCTC CTGGGGGCAC AGGAATGTCG | 1980 |
| AAGCAAATGC CATGGACATG GGGTCTGTGA CAGCAACAGG CACTGCTACT GTGAGGAGGG | 2040 |
| CTGGGCACCC CCTGACTGCA CCACTCAGCT CAAAGCAACC AGCTCCCTGA CCACAGGGCT | 2100 |
| GCTCCTCAGC CTCCTGGTCT TATTGGTCCT GGTGATGCTT GGTGCCGGCT ACTGGTACCG | 2160 |
| TGCCCGCCTG CACCAGCGAC TCTGCCAGCT CAAGGGACCC ACCTGCCAGT ACAGGGCAGC | 2220 |
| CCAATCTGGT CCCTCTGAAC GGCCAGGACC TCCGCAGAGG GCCCTGCTGG CACGAGGCAC | 2280 |
| TAAGTCTCAG GGGCCAGCCA AGCCCCCACC CCCAAGGAAG CCACTGCCTG CCGACCCCCA | 2340 |
| GGGCCGGTGC CCATCGGGTG ACCTGCCCGG CCCAGGGGCT GGAATCCCGC CCCTAGTGGT | 2400 |
| ACCCTCCAGA CCAGCGCCAC CGCCTCCGAC AGTGTCCTCG CTCTACCTCT GACCTCTCCG | 2460 |
| GAGGTTCCGC TGCCTCCAAG CCGGACTTAG GGCTTCAAGA GGCGGGCGTG CCCTCTGGAG | 2520 |
| TCCCCTACCA TGACTGAAGG CGCCAGAGAC TGGCGGTGTC TTAAGACTCC GGGCACCGCC | 2580 |
| ACGCGCTGTC AAGCAACACT CTGCGGACCT GCCGGCGTAG TTGCAGCGGG GGCTTGGGGA | 2640 |
| GGGGCTGGGG GTTGGACGGG ATTGAGGAAG GTCCGCACAG CCTGTCTCTG CTCAGTTGCA | 2700 |
| ATAAACGTGA CATCTTGGGA GCGTTAAAAA AAAAAAAAAA | |

Seq ID NO: 112 Protein sequence:
Protein Accession #: NP_003806.2

| | |
|---|---|
| 1 11 21 31 41 51 | |
| \| \| \| \| \| \| | |
| MRLALLWALG LLGAGSPLPS WPLPNIGGTE EQQAESEKAP RBPLEPQVLQ DDLPISLKKV | 60 |
| LQTSLPEPLR IKLELDGDSH ILELLQNREL VPGRPTLVWY QPDGTRVVSE GHTLENCCYQ | 120 |
| GRVRGYAGSW VSICTCSGLR GLVVLTPERS YTLEQGPGDL QGPPIISRIQ DLHLPGHTCA | 180 |
| LSWRESVHTQ TPPEHPLGQR HIRRRRDVVT ETKTVELVIV ADHSEAQKYR DFQHLLNRTL | 240 |
| EVALLLDTFF RPLNVRVALV GLEAWTQRDL VEISPNPAVT LENFLHWRRA HLLPRLPHDS | 300 |
| AQLVTGTSFS GPTVGMA1QN SICSPDFSGG VNMDHSTSIL GVASSIAHEL GHSLGLDHDL | 360 |
| PGNSCPCPGP APAKTCIMEA STDFLPGLNF SNCSRRALEK ALLDGMGSCL FERLPSLPPM | 420 |
| AAFCGNMFVE PGEQCDCGFL DDCVDPCCDS LTCQLRPGAQ CASDGPCCQN CQLRTSGWQC | 480 |
| RPTRGDCDLP EFCPGDSSQC PPDVSLGDGE PCAGGQAVCM HGRCASYAQQ CQSLWGPGAQ | 540 |
| PAAPLCLQTA NTRGNAFGSC GRGPSGSYVS CTPRDAICGQ LQCQTGRTQP LLGS1RDLLW | 600 |
| ETIDVNGTEL NCSWVHLDLG SDVAQPLLTL PGTACGPGLV CIDHRCQRVD LLGAQECRSK | 660 |
| CHGHGVCDSN RHCYCEEGWA PPDCTTQLKA TSSLTTGLLL SLLVLLVLVM LGAGYWYRAR | 720 |
| LHQRLCQLKG PTCQYRAAQS GPSERPGPPQ RALLARGTKS QGFAKPPPPR KPLPADPQGR | 780 |
| CPSGDLPGPG AGIPPLVVPS RPAPPPPTVS SLYL | |

Seq ID NO: 113 DNA sequence
Nucleic Acid Accession #: NM_002416
Coding sequence: 40-417 (underlined sequences correspond to start and
stop codons)

| | |
|---|---|
| 1 11 21 31 41 51 | |
| \| \| \| \| \| \| | |
| ATCCAATACA GGAGTGACTT GGAACTCCAT TCTATCACTA TGAAGAAAAG TGGTGTTCTT | 60 |
| TTCCTCTTGG GCATCATCTT GCTGGTTCTG ATTGGAGTGC AAGGAACCCC AGTAGTGAGA | 120 |
| AAGGGTCGCT GTTCCTGCAT CAGCACCAAC CAAGGGACTA TCCACCTACA ATCCTTGAAA | 180 |
| GACCTTAAAC AATTTGCCCC AAGCCCTTCC TGCGAGAAAA TTGAAATCAT TGCTACACTG | 240 |
| AAGAATGGAG TTCAAACATG TCTAAACCCA GATTCAGCAG ATGTGAAGGA ACTGATTAAA | 300 |
| AAGTGGGAGA AACAGGTCAG CCAAAAGAAA AAGCAAAAGA ATGGGAAAAA ACATCAAAAA | 360 |
| AAGAAAGTTC TGAAAGTTCG AAAATCTCAA CGTTCTCGTC AAAAGAAGAC TACATAAGAG | 420 |
| ACCACTTCAC CAATAAGTAT TCTGTGTTAA AAATGTTCTA TTTTAATTAT ACCGCTATCA | 480 |
| TTCCAAAGGA GGATGGCATA TAATACAAAG GCTTATTAAT TTGACTAGAA AATTTAAAAC | 540 |
| ATTACTCTGA AATTGTAACT AAAGTTAGAA AGTTGATTTT AAGAATCCAA ACGTTAAGAA | 600 |
| TTGTTAAAGG CTATGATTGT CTTTGTTCTT CTACCACCCA CCAGTTGAAT TTCATCATGC | 660 |
| TTAAGGCCAT GATTTTAGCA ATACCCATGT CTACACAGAT GTTCACCCAA CCACATCCCA | 720 |
| CTCACAACAG CTGCCTGGAA GAGCAGCCCT AGGCTTCCAC GTACTGCAGC CTCCAGAGAG | 780 |
| TATCTGAGGC ACATGTCAGC AAGTCCTAAG CCTGTTAGCA TGCTGGTGAG CCAAGCAGTT | 840 |
| TGAAATTGAG CTGGACCTCA CCAAGCTGCT GTGGCCATCA ACCTCTGTAT TTGAATCAGC | 900 |
| CTACAGGCCT CACACACAAT GTGTCTGAGA GATTCATGCT GATTGTTATT GGGTATCACC | 960 |
| ACTGGAGATC ACCAGTGTGT GGCTTTCAGA GCCTCCTTTC TGGCTTTGGA AGCCATGTGA | 1020 |
| TTCCATCTTG CCCGCTCAGG CTGACCACTT TATTTCTTTT TGTTCCCCTT TGCTTCATTC | 1080 |
| AAGTCAGCTC TTCTCCATCC TACCACAATG CAGTGCCTTT CTTCTCTCCA GTGCACCTGT | 1140 |
| CATATGCTCT GATTTATCTG AGTCAACTCC TTTCTCATCT TGTCCCCAAC ACCCCACAGA | 1200 |
| AGTGCTTTCT TCTCCCAATT CATCCTCACT CAGTCCAGCT TAGTTCAAGT CCTGCCTCTT | 1260 |
| AAATAAACCT TTTTGGACAC ACAAATTATC TTAAAACTCC TGTTTCACTT GGTTCAGTAC | 1320 |
| CACATGGGTG AACACTCAAT GGTTAACTAA TTCTTGGGTG TTTATCCTAT CTCTCCAACC | 1380 |
| AGATTGTCAG CTCCTTGAGG GCAAGAGCCA CAGTATATTT CCCTGTTTCT TCCACAGTGC | 1440 |
| CTAATAATAC TGTGGAACTA GGTTTTAATA ATTTTTTAAT TGATGTTGTT ATGGGCAGGA | 1500 |
| TGGCAACCAG ACCATTGTCT CAGAGCAGGT GCTGGCTCTT TCCTGGCTAC TCCATGTTGG | 1560 |
| CTAGCCTCTG GTAACCTCTT ACTTATTATC TTCAGGACAC TCACTACAGG GACCAGGGAT | 1620 |
| GATGCAACAT CCTTGTCTTT TTATGACAGG ATGTTTGCTC AGCTTCTCCA ACAATAAGAA | 1680 |
| GCACGTGGTA AAACACTTGC GGATATTCTG GACTGTTTTT AAAAAATATA CAGTTTACCG | 1740 |
| AAAATCATAT AATCTTACAA TGAAAAGGAC TTTATAGATC AGCCAGTGAC CAACCTTTTC | 1800 |
| CCAACCATAC AAAAATTCCT TTTCCCGAAG GAAAAGGGGT TTCTCAATAA GCCTCAGCTT | 1860 |
| TCTAAGATCT AACAAGATAG CCACCGAGAT CCTTATCGAA ACTCATTTTA GGCAAATATG | 1920 |
| AGTTTTATTG TCCGTTTACT TGTTTCAGAG TTTGTATTGT GATTATCAAT TACCACACCA | 1980 |
| TCTCCCATGA AGAAAGGGAA CGGTGAAGTA CTAAGCGCTA GAGGAAGCAG CCAAGTCGGT | 2040 |
| TAGTGGAAGC ATTATTGGTG CCCAGTTAGC CTCTGCAGGA TGTGGAAACC TCCTTCCAGG | 2100 |
| GGAGGTTCAG TGAATTGTGT AGGAGAGGTT GTCTGTGGCC AGAATTTAAA CCTATACTCA | 2160 |
| CTTTCCCAAA TTGAATCACT GCTCACACTG CTGATGATTT AGAGTGCTGT CCGGTGGAGA | 2220 |
| TCCCACCCGA ACGTCTTATC TAATCATGAA ACTCCCTAGT TCCTTCATGT AACTTCCCTG | 2280 |
| AAAAATCTAA GTGTTTCATA AATTTGAGAG TCTGTGACCC ACTTACCTTG CATCTCACAG | 2340 |
| GTAGACAGTA TATAACTAAC AACCAAAGAC TACATATTGT CACTGACACA CACGTTATAA | 2400 |
| TCATTTATCA TATATATACA TACATGCATA CACTCTCAAA GCAAATAATT TTTCACTTCA | 2460 |
| AAACAGTATT GACTTGTATA CCTTGTAATT TGAAATATTT TCTTTGTTAA AATAGAATGG | 2520 |
| TATCAATAAA TAGACCATTA ATCAG | |

Seq ID NO: 114 Protein sequence:
Protein Accession #: NP_002407

| | |
|---|---|
| 1 11 21 31 41 51 | |
| \| \| \| \| \| \| | |
| MKKSGVLFLL GIILLVLIGV QGTPVVRXGR CSCISTNQGT IHLQSLKDLK QFAPSPSCEK | 60 |
| IEIIATLKNG VQTCLNPDSA DVKELIKKWE KQVSQKKKQK NGKKHQKKKV LKVRXSQRSR | 120 |
| QKKTT | |

Seq ID NO: 115 DNA sequence
Nucleic Acid Accession #: NM_003238.1
Coding sequence: 182-1426 (underlined sequences correspond to start
and stop codons)

| | |
|---|---|
| 1 11 21 31 41 51 | |
| \| \| \| \| \| \| | |
| CAAGCAGGAT ACGTTTTTCT GTTGGGCATT GACTAGATTG TTTGCAAAAG TTTCGCATCA | 60 |
| AAAACAAACA ACAACAACAA AAAACCAAAC AACTCTCCTT GATCTATACT TTGAGAATTG | 120 |
| TTGATTTCTT TTTTTTTATT CTGACTTTTA AAAACAACTT TTTTTTCCAC TTTTTTAAAA | 180 |
| AATGCACTAC TGTGTGCTGA GCGCTTTTCT GATCCTGCAT CTGGTCACGG TCGCGCTCAG | 240 |
| CCTGTCTACC TGCAGCACAC TCGATATGGA CCAGTTCATG CGCAAGAGGA TCGAGGCGAT | 300 |
| CCGCGGGCAG ATCCTGAGCA AGCTGAAGCT CACCAGTCCC CCAGAAGACT ATCCTGAGCC | 360 |
| CGAGGAATTC CCCCCGGAGG TGATTTCCAT CTACAACAGC ACCAGGGACT TGCTCCAGGA | 420 |
| GAAGGCGAGC CGGAGGGCGG CCGCCTGCGA GCGCGAGAGG AGCGACGAAG AGTACTACGC | 480 |
| CAAGGAGGTT TACAAAATAG ACATGCCGCC CTTCTTCCCC TCCGAAAATG CCATCCCGCC | 540 |
| CACTTTCTAC AGACCCTACT TCAGAATTGT TCGATTTGAC GTCTCAGCAA TGGAGAAGAA | 600 |
| TGCTTCCAAT TTGGTGAAAG CAGAGTTCAG AGTC1TTCGT TTGCAGAACC CAAAAGCCAG | 660 |
| AGTGCCTGAA CAACGGATTG AGCTATATCA GATTCTCAAG TCCAAAGATT TAACATCTCC | 720 |
| AACCCAGCGC TACATCGACA GCAAAGTTGT GAAAACAAGA GCAGAAGGCG AATGGCTCTC | 780 |
| CTTCGATGTA ACTGATGCTG TTCATGAATG GCTTCACCAT AAAGACAGGA ACCTGGGATT | 840 |
| TAAAATAAGC TTACACTGTC CCTGCTGCAC TTTTGTACCA TCTAATAATT ACATCATCCC | 900 |
| AAATAAAAGT GAAGAACTAG AAGCAAGATT TGCAGGTATT GATGGCACCT CCACATATAC | 960 |
| CAGTGGTGAT CAGAAAACTA TAAAGTCCAC TAGGAAAAAA AACAGTGGGA AGACCCCACA | 1020 |
| TCTCCTGCTA ATGTTATTGC CCTCCTACAG ACTTGAGTCA CAACAGACCA ACCGGCGGAA | 1080 |
| GAAGCGTGCT TTGGATGCGG CCTATTGCTT TAGAAATGTG CAGGATAATT GCTGCCTACG | 1140 |
| TCCACTTTAC ATTGATTTCA AGAGGGATCT AGGGTGGAAA TGGATACACG AACCCAAAGG | 1200 |
| GTACAATGCC AACTTCTGTG CTGGAGCATG CCCGTATTTA TGGAGTTCAG ACACTCAGCA | 1260 |
| CAGCAGGGTC CTGAGCTTAT ATAATACCAT AAATCCAGAA GCATCTGCTT CTCCTTGCTG | 1320 |
| CGTGTCCCAA GATTTAGAAC CTCTAACCAT TCTCTACTAC ATTGGCAAAA CACCCAAGAT | 1380 |
| TGAACAGCTT TCTAATATGA TTGTAAAGTC TTGCAAATGC AGCTAAAATT CTTGGAAAAG | 1440 |
| TGGCAAGACC AAAATGACAA TGATGATGAT AATGATGATG ACGACGACAA CGATGATGCT | 1500 |
| TGTAACAAGA AAACATAAGA GAGCCTTGGT TCATCAGTGT TAAAAAATTT TTGAAAAGGC | 1560 |
| GGTACTAGTT CAGACACTTT GGAAGTTTGT GTTCTGTTTG TTAAAACTGG CATCTGACAC | 1620 |
| AAAAAAAGTT GAAGGCCTTA TTCTACATTT CACCTACTTT GTAAGTGAGA GAGACAAGAA | 1680 |
| GCAAATTTTT TTAAA | |

Seq ID NO: 116 Protein seouence:
Protein Accession #: NP_003229.1

| | |
|---|---|
| 1 11 21 31 41 51 | |
| \| \| \| \| \| \| | |
| MHYCVLSAFL ILHLVTVALS LSTCSTLDMD QFMRXRIEAI RGQILSKLKL TSPPEDYPEP | 60 |
| EEVPPEVISI YNSTRDLLQE KASRRAAACE RERSDEEYYA KEVYKIDMPP FFPSENAIPP | 120 |
| TFYRPYFRIV RFDVSAMEKN ASNLVKAEFR VFRLQNPKAR VPEQRIELYQ ILKSKDLTSP | 180 |
| TQRYIDSKVV KTRAEGEWLS FDVTDAVHEW LHHKDRNLGF KISLHCPCCT FVPSNNYIIP | 240 |
| NKSEELEARF AGIXITSTYT SGDQKTIKST RKKNSGKTPH LLLMLLPSYR LESQQTNRRK | 300 |
| KRALDAAYCF RNVQDNCCLR PLYIDFKRDL GWKWIHEPKG YNANFCAGAC PYLWSSDTQH | 360 |
| SRVLSLYNTI NPEASASPCC VSQDLEPLTI LYYIGKTPKI EQLSNM1VKS CKCS | |

Seq ID NO: 117 DNA sequence
Nucleic Acid Accession #: NM_000095.1
Coding sequence: 26-2299 (underlined sequences correspond to start and
stop codons)

| | |
|---|---|
| 1 11 21 31 41 51 | |
| \| \| \| \| \| \| | |
| CAGCACCCAG CTCCCCGCCA CCGCCATGGT CCCCGACACC GCCTGCGTTC TTCTGCTCAC | 60 |
| CCTGGCTGCC CTCGGCGCGT CCGGACAGGG CCAGAGCCCG TTGGGCTCAG ACCTGGGCCC | 120 |
| GCAGATGCTT CGGGAACTGC AGGAAACCAA CGCGGCGCTG CAGGACGTGC GGGACTGGCT | 180 |
| GCGGCAGCAG GTCAGGGAGA TCACGTTCCT GAAAAACACG GTGATGGAGT GTGACGCGTG | 240 |
| CGGGATGCAG CAGTCAGTAC GCACCGGCCT ACCCAGCGTG CGGCCCCTGC TCCACTGCGC | 300 |
| GCCCGGCTTC TGCTTCCCCG GCGTGGCCTG CATCCAGACG GAGAGCGGCG GCCGCTGCGG | 360 |
| CCCCTGCCCC GCGGGCTTCA CGGGCAACGG CTCGCACTGC ACCGACGTCA ACGAGTGCAA | 420 |
| CGCCCACCCC TGCTTCCCCC GAGTCCGCTG TATCAACACC AGCCCGGGGT TCCGCTGCGA | 480 |
| GGCTfGCCCG CCGGGGTACA GCGGCCCCAC CCACCAGGGC GTGGGGCTGG CTTTCGCCAA | 540 |
| GGCCAACAAG CAGGTTTGCA CGGACATCAA CGAGTGTGAG ACCGGGCAAC ATAACTGCGT | 600 |
| CCCCAACTCC GTGTGCATCA ACACCCGGGG CTCCTTCCAG TGCGGCCCGT GCCAGCCCGG | 660 |
| CTTCGTGGGC GACCAGGCGT CCGGCTGCCA GCGCGGCGCA CAGCGCTTCT GCCCCGACGG | 720 |
| CTCGCCCAGC GAGTGCCACG AGCATGCAGA CTGCGTCCTA GAGCGCGATG GCTCGCGGTC | 780 |
| GTGCGTGTGT CGCGTTGGCT GGGCCGGCAA CGGGATCCTC TGTGGTCGCG ACACTGACCT | 840 |
| AGACGGC1TC CCGGACGAGA AGCTGCGCTG CCCGGAGCCG CAGTGCCGTA AGGACAACTG | 900 |
| CGTGACTGTG CCCAACTCAG GGCAGGAGGA TGTGGACCGC GATGGCATCG GAGACGCCTG | 960 |
| CGATCCGGAT GCCGACGGGG ACGGGGTCCC CAATGAAAAG GACAACTGCC CGCTGGTGCG | 1020 |
| GAACCCAGAC CAGCGCAACA CGGACGAGGA CAAGTGGGGC GATGCGTGCG ACAACTGCCG | 1080 |
| GTCCCAGAAG AACGACGACC AAAAGGACAC AGACCAGGAC GGCCGGGGCG ATGCGTGCGA | 1140 |
| CGACGACATC GACGGCGACC GGATCCGCAA CCAGGCCGAC AACTGCCCTA GGGTACCCAA | 1200 |
| CTCAGACCAG AAGGACAGTG ATGGCGATGG TATAGGGGAT GCCTGTGACA ACTGTCCCCA | 1260 |
| GAAGAGCAAC CCGGATCAGG CGGATGTGGA CCACGACTTT GTGGGAGATG CTTGTGACAG | 1320 |
| CGATCAAGAC CAGGATGGAG ACGGACATCA GGACTCTCGG GACAACTGTC CCACGGTGCC | 1380 |
| TAACAGTGCC CAGGAGGACT CAGACCACGA TGGCCAGGGT GATGCCTGCG ACGACGACGA | 1440 |
| CGACAATGAC GGAGTCCCTG ACAGTCGGGA CAACTGCCGC CTGGTGCCTA ACCCCGGCCA | 1500 |
| GGAGGACGCG GACAGGGACG GCGTGGGCGA CGTGTGCCAG GACGACTTTG ATGCAGACAA | 1560 |
| GGTGGTAGAC AAGATCGACG TGTGTCCGGA GAACGCTGAA GTCACGCTCA CCGACTTCAG | 1620 |
| GGCCTTCCAG ACAGTCGTGC TGGACCCGGA GGGTGACGCG CAGATTGACC CCAACTGGGT | 1680 |
| GGTGCTCAAC CAGGGAAGGG AGATCGTGCA GACAATGAAC AGCGACCCAG GCCTGGCTGT | 1740 |
| GGGTTACACT GCCTTCAATG GCGTGGACTT CGAGGGCACG TTCCATGTGA ACACGGTCAC | 1800 |
| GGATGACGAC TATGCGGGCT TCATCTTTGG CTACCAGGAC AGCTCCAGCT TCTACGTGGT | 1860 |
| CATGTGGAAG CAGATGGAGC AAACGTATTG GCAGGCGAAC CCCTTCCGTG CTGTGGCCGA | 1920 |
| GCCTGGCATC CAACTCAAGG CTGTGAAGTC TTCCACAGGC CCCGGGGAAC AGCTGCGGAA | 1980 |
| CGCTCTGTGG CATACAGGAG ACACAGAGTC CCAGGTGCGG CTGCTGTGGA AGGACCCGCG | 2040 |
| AAACGTGGGT TGGAAGGACA AGAAGTCCTA TCGTTGGTTC CTGCAGCACC GGCCCCAAGT | 2100 |
| GGGCTACATC AGGGTGCGAT TCTATGAGGG CCCTGAGCTG GTGGCCGACA GCAACGTGGT | 2160 |
| CTTGGACACA ACCATGCGGG GTGGCCGCCT GGGGGTCTTC TGCTTCTCCC AGGAGAACAT | 2220 |
| CATCTGGGCC AACCTGCGIT ACCGCTGCAA TGACACCATC CCAGAGGACT ATGAGACCCA | 2280 |
| TCAGCTGCGG CAAGCCTAGG GACCAGGGTG AGGACCCGCC GGATGACAGC CACCCTCACC | 2340 |
| GCGGCTGGAT GGGGGCTCTG CACCCAGCCC AAGGGGTGGC CGTCCTGAGG GGGAAGTGAG | 2400 |
| AAGGGCTCAG AGAGGACAAA ATAAAGTGTG TGTGCAGGG | |

Seq ID NO: 118 Protein sequence:
Protein Accession #: NP_000086.1

| | |
|---|---|
| 1 11 21 31 41 51 | |
| \| \| \| \| \| \| | |
| MVPDTACVLL LTLAALGASG QGQSPLGSDL GPQMLRELQE TNAALQDVRD WLRQQVREIT | 60 |
| FLKNTVMECD ACGMQQSVRT GLPSVRPLLH CAPGFCFPGV ACIQTESGGR CGPCPAGFTG | 120 |
| NGSHCTDVNE CNAHPCFPRV RCINTSPGFR CEACPPGYSG PTHQGVGLAF AKANKQVCTD | 180 |
| INECETGQHN CVPNSVCINT RGSPQCGPCQ PGFVGDQASG CQRGAQRFCP DGSPSECHEH | 240 |
| ADCVLERDGS RSCVCRVGWA GNGILCGRDT DLDGFPDEKL RCPEPQCRKD NCVTVPNSGQ | 300 |
| EDVDRDGIGD ACDPDADGDG VPNEKDNCHL VRNPDQRTTD EDKWGDACDN CRSQKNDDQK | 360 |
| DTDQDGRGDA CDDDIDGDRI RNQADNCPRV PNSDQKDSDG DGIGDACDNC PQKSNPDQAD | 420 |
| VDHDFVGDAC DSDQDQDGDG HQDSRDNCPT VPNSAQEDSD HDGQGDACDD DDDNDGVPDS | 480 |
| RDNCRLVPNP GQEDADRDGV GDVCQDDFDA DKVVDKIDVC PENAEVTLTD FRAFQTVVLD | 540 |
| PEGDAQIDPN WVVLNQGREI VQTMNSDPGL AVGYTAFNGV DFEGTFHVNT VTDDDYAGFI | 600 |
| FGYQDSSSFY VVMWKQMEQT YWQANPFRAV AEPGIQLKAV KSSTGPGEQL RNALWHTGDT | 660 |
| ESQVRLLWKD PRNVGWKDKK SYRWFLQHRP QVGYIRVRFY EGPELVADSN VVLDTTHRGG | 720 |
| RLGVFCFSQE NIIWANLRYR CNDTIPEDYE THQLRQA | |

Seq ID NO: 119 DNA sequence
Nucleic Acid Accession #: NM_01421.1
Coding sequence: 157-1479 (underlined sequences correspond to start
and stop codons)

| | |
|---|---|
| 1 11 21 31 41 51 | |
| \| \| \| \| \| \| | |
| GGGACAGGGC TGAGGATGAG GAGAACCCTG GGGACCCAGA AGACCGTGCC TTGCCCGGAA | 60 |
| GTCCTGCCTG TAGGCCTGAA GGACTTGCCC TAACAGAGCC TCAACAACTA CCTGGTGATT | 120 |
| CCTACTTCAG CCCCTTGGTG TGAGCAGCTT CTCAACATGA ACTACAGCCT CCACTTGGCC | 180 |
| TTCGTGTGTC TGAGTCTCTT CACTGAGAGG ATGTGCATCC AGGGGAGTCA GTTCAACGTC | 240 |
| GAGGTCGGCA GAAGTGACAA GCTTTCCCTG CCTGGCTTTG AGAACCTCAC AGCAGGATAT | 300 |
| AACAAATTTC TCAGGCCCAA TTTTGGTGGA GAACCCGTAC AGATAGCGCT GACTCTGGAC | 360 |
| ATTGCAAGTA TCTCTAGCAT TTCAGAGAGT AACATGGACT ACACAGCCAC CATATACCTC | 420 |
| CGACAGCGCT GGATGGACCA GCGGCTGGTG TTTGAAGGCA ACAAGAGCTT CACTCTGGAT | 480 |
| GCCCGCCTCG TGGAGTTCCT CTGGGTGCCA GATACTTACA TTGTGGAGTC CAAGAAGTCC | 540 |
| TTCCTCCATG AAGTCACTGT GGGAAACAGG CTCATCCGCC TCTTCTCCAA TGGCACGGTC | 600 |
| CTGTATGCCC TCAGAATCAC GACAACTGTT GCATGTAACA TGGATCTGTC TAAATACCCC | 660 |
| ATGGACACAC AGACATGCAA GTTGCAGCTG GAAAGCTGGG GCTATGATGG AAATGATGTG | 720 |
| GAGTTCACCT GGCTGAGAGG GAACGACTCT GTGCGTGGAC TGGAACACCT GCGGCTTGCT | 780 |
| CAGTACACCA TAGAGCGGTA TTTCACCTTA GTCACCAGAT CGCAGCAGGA GACAGGAAAT | 840 |
| TACACTAGAT TGGTCTTACA GTTTGAGCTT CGGAGGAATG TTCTGTATTT CATTTTGGAA | 900 |
| ACCTACGTTC CTTCCACTTT CCTGGTGGTG TTGTCCTGGG TTTCATTTTG GATCTCTCTC | 960 |
| GATTCAGTCC CTGCAAGAAC CTGCATTGGA GTGACGACCG TGTTATCAAT GACCACACTG | 1020 |
| ATGATCGGGT CCCGCACTTC TCTTCCCAAC ACCAACTGCT TCATCAAGGC CATCGATGTG | 1080 |
| TACCTGGGGA TCTGCTTTAG CTTTGTGTTT GGGGCCTTGC TAGAATATGC AGTTGCTCAC | 1140 |
| TACAGTTCCT TACAGGAGAT GGCAGCCAAA GATAGGGGGA CAACAAAGGA AGTAGAAGAA | 1200 |
| GTCAGTATTA CTAATATCAT CAACAGCTCC ATCTCCAGCT TTAAACGGAA GATCAGCTTT | 1260 |
| GCCAGCATTG AAATTTCCAG CGACAACGTT GACTACAGTG ACTTGACAAT GAAAACCAGC | 1320 |
| GACAAGTTCA AGTTTGTCTT CCGAGAAAAG ATGGGCAGGA TTGTTGATTA TTTCACAATT | 1380 |
| CAAAACCCCA GTAATGTTGA TCACTATTCC AAACTACTGT TTCCTTTGAT TTTTATGCTA | 1440 |
| GCCAATGTAT TTTACTGGGC ATACTACATG TATTTTTGAG TCAATGTTAA ATTTCTTGCA | 1500 |
| TGCCATAGGT CTTCAACAGG ACAAGATAAT GATGTAAATG GTATTTTAGG CCAAGTGTGC | 1560 |
| ACCCACATCC AATGGTGCTA CAAGTGACTG AAATAATATT TGAGTCTTTC TGCTCAAAGA | 1620 |
| ATGAAGCTCC AACCATTGTT CTAAGCTGTG TAGAAGTCCT AGCATTATAG GATCTTGTAA | 1680 |
| TAGAAACATC AGTCCATTCC TCTTTCATCT TAATCAAGGA CATTCCCATG GAGCCCAAGA | 1740 |
| TTACAAATGT ACTCAGGGCT GTTTATTCGG TGGCTCCCTG GTTTGCATTT ACCTCATATA | 1800 |
| AAGAATGGGA AGGAGACCAT TGGGTAACCC TCAAGTGTCA GAAGTTGTTT CTAAAGTAAC | 1860 |
| TATACATGTT TTTTACTAAA TCTCTGCAGT GCTTATAAAA TACATTGTTG CCTATTTAGG | 1920 |
| GAGTAACATT TTCTAGTTTT TGTTTCTGGT TAAAATGAAA TATGGGCTTA TGTCAATTCA | 1980 |
| TTGGAAGTCA ATGCACTAAC TCAATACCAA GATGAGTTTT TAAATAATGA ATATTATTTA | 2040 |
| ATACCACAAC AGAATTATCC CCAATTTCCA ATAAGTCCTA TCATTGAAAA TTCAAATATA | 2100 |
| AGTGAAGAAA AAATTAGTAG ATCAACAATC TAAACAAATC CCTCGGTTCT AAGATACAAT | 2160 |
| GGATTCCCCA TACTGGAAGG ACTCTGAGGC TTTATTCCCC CACTATGCAT ATCTTATCAT | 2220 |
| TTTATTATTA TACACACATC CATCCTAAAC TATACTAAAG CCCTTTTCCC ATGCATGGAT | 2280 |
| GGAAATGGAA GATTTTTTTG TAACTTGTTC TAGAAGTCTT AATATGGGCT GTTGCCATGA | 2340 |
| AGGCTTGCAG AATTGAGTCC ATTTTCTAGC TGCCTTTATT CACATAGTGA TGGGGTACTA | 2400 |
| AAAGTACTGG GTTGACTCAG AGAGTCGCTG TCATTCTGTC ATTGCTGCTA CTCTAACACT | 2460 |
| GAGCAACACT CTCCCAGTGG CAGATCCCCT GTATCATTCC AAGAGGAGCA TTCATCCCTT | 2520 |
| TGCTCTAATG ATCAGGAATG ATGCTTATTA GAAAACAAAC TGCTTGACCC AGGAACAAGT | 2580 |
| GGCTTAGCTT AAGTAAACTT GGCTTTGCTC AGATCCCTGA TCCTTCCAGC TGGTCTGCTC | 2640 |
| TGAGTGGCTT ATCCCGCATG AGCAGGAGCG TGCTGGCCCT GAGTACTGAA CTTTCTGAGT | 2700 |
| AACAATGAGA CACGTTACAG AACCTATGTT CAGGTTGCGG GTGAGCTGCC CTCTCCAAAT | 2760 |
| CCAGCCAGAG ATGCACATTC CTCGGCCAGT CTCAGCCAAC AGTACCAAAA GTGATTTTTG | 2820 |
| AGTGTGCCAG GGTAAAGGCT TCCAGTTCAG CCTCAGTTAT TTTAGACAAT CTCGCCATCT | 2880 |
| TTAATTTCTT AGCTTCCTGT TCTAATAAAT GCACGGCTTT ACCTTTCCTG TCAGAAATAA | 2940 |
| ACCAAGGCTC TAAAAGATGA TTTCCCTTCT GTAACTCCCT AGAGCCACAG GTTCTCATTC | 3000 |
| CTITTCCCAT TATACTTCTC ACAATTCAGT TTCTATGAGT TTGATCACCT GATTTTTTTA | 3060 |
| ACAAAATATT TCTAACGGGA ATGGGTGGGA GTGCTGGTGA AAAGAGATGA AATGTGGTTG | 3120 |
| TATGAGCCAA TCATATTTGT GATTITTTAA AAAAAGTTTA AAAGGAAATA TCTGTTCTGA | 3180 |
| AACCCCACTT AAGCATTGTT TTTATATAAA AACAATGATA AAGATGTGAA CTGTGAAATA | 3240 |
| AATATACCAT ATTAGCTACC CACC | |

Seq ID NO: 120 Protein sequence:
Protein Accession #: NP_055026.1

| | |
|---|---|
| 1 11 21 31 41 51 | |
| \| \| \| \| \| \| | |
| MNYSLHLAFV CLSLFTERMC IQGSQFNVEV GRSDKLSLPG FENLTAGYNK FLRPNFGGEP | 60 |
| VQIALTLDIA SISSISESNM DYTATIYLRQ RWMDQRLVFE GNKSFTLDAR LVEFLWVPDT | 120 |
| YIVESKXSFL HEVTVGNRLI RIFSNGTVLY ALRITTTVAC NMDLSKYPMD TQTCKLQLES | 180 |
| WGYDGNDVEF TWLRGNDSVR GLEHLRLAQY TIERYFTLVT RSQQETGNYT RLVLQFELRR | 240 |
| NVLYFILETY VPSTFLVVLS WVSFW1SLDS VPARTCIGVT TVLSMTTLMI GSRTSLPNTN | 300 |
| CFIKAIDVYL GICFSFVFGA LLEYAVAHYS SLQQMAAKDR GTTKEVEEVS ITNIINSSIS | 360 |
| SFKRKISFAS IEISSDNVDY SDLTMKTSDK EKEVFREKMG RIVDYFTIQN PSNVDHYSKL | 420 |
| LFPLIFMLAN VFYWAYYMYF | |

Seq ID NO: 121 DNA sequence
Nucleic Acid Accession #: NM_001854
Coding sequence: 163-5582 (underlined sequences correspond to start
and stop codons)

| | |
|---|---|
| 1 11 21 31 41 51 | |
| \| \| \| \| \| \| | |
| AACCATCAAA TTTAGAAGAA AAAGCCCTTT GACTTTTTCC CCCTCTCCCT CCCCAATGGC | 60 |
| TGTGTAGCAA ACATCCCTGG CGATACCTTG GAAAGGACGA AGTTGGTCTG CAGTCGCAAT | 120 |
| TTCGTGGGTT GAGTTCACAG TTGTGAGTGC GGGGCTCGGA GATGGAGCCG TGGTCCTCTA | 180 |
| GGTGGAAAAC GAAACGGTGG CTCTGGGATT TCACCGTAAC AACCCTCGCA TTGACCTTCC | 240 |
| TCTTCCAAGC TAGAGAGGTC AGAGGAGCTG CTCCAGTTGA TGTACTAAAA GCACTAGATT | 300 |
| ITCACAATTC TCCAGAGGGA ATATCAAAAA CAACGGGATT TTGCACAAAC AGAAAGAATT | 360 |
| CTAAAGGCTC AGATACTGCT TACAGAGTTT CAAAGCAAGC ACAACTCAGT GCCCCAACAA | 420 |
| AACAGTTATT TCCAGGTGGA ACTTTCCCAG AAGACTTTTC AATAGTATTT ACAGTAAAAC | 480 |
| CAAAAAAAGG AATTCAGTCT TTCCTTTTAT CTATATATAA TGAGCATGGT ATTCAGCAAA | 540 |
| TTGGTGTTGA GGTTGGGAGA TCACCTGTTT TTCTGTTTGA AGACCACACT GGAAAACCTG | 600 |
| CCCCAGAAGA CTATCCCCTC TTCAGAACTG TTAACATCGC TGACGGGAAG TGGCATCGGG | 660 |
| TAGCAATCAG CGTGGAGAAG AAAACTGTGA CAATGATTGT TGATTGTAAG AAGAAAACCA | 720 |
| CGAAACCACT TGATAGAAGT GAGAGAGCAA TTGTTGATAC CAATGGAATC ACGGTTTTTG | 780 |
| GAACAAGGAT TTTGGATGAA GAAGTTTTTG AGGGGGACAT TCAGCAGTTT TTGATCACAG | 840 |
| GTGATCCCAA GGCAGCATAT GACTACTGTG AGCATTATAG TCCAGACTGT GACTCTTCAG | 900 |
| CACCCAAGGC TGCTCAAGCT CAGGAACCTC AGATAGATGA GTATGCACCA GAGGATATAA | 960 |
| TCGAATATGA CTATGAGTAT GGGGAAGCAG AGTATAAAGA GGCTGAAAGT GTAACAGAGG | 1020 |
| GACCCACTGT AACTGAGGAG ACAATAGCAC AGACGGAGGC AAACATCGTT GATGATTTTC | 1080 |
| AAGAATACAA CTATGGAACA ATGGAAAGTT ACCAGACAGA AGCTCCTAGG CATGTTTCTG | 1140 |
| GGACAAATGA GCCAAATCCA GTTGAAGAAA TATTTACTGA AGAATATCTA ACGGGAGAGG | 1200 |
| ATTATGATTC CCAGAGGAAA AATTCTGAGG ATACACTATA TGAAAACAAA GAAATAGACG | 1260 |
| GCAGGGATTC TGATCTTCTG GTAGATGGAG ATTTAGGCGA ATATGATTTT TATGAATATA | 1320 |
| AAGAATATGA AGATAAACCA ACAAGCCCCC CTAATGAAGA ATTTGGTCCA GGTGTACCAG | 1380 |
| CAGAAACTGA TATTACAGAA ACAAGCATAA ATGGCCATGG TGCATATGGA GAGAAAGGAC | 1440 |
| AGAAAGGAGA ACCAGCAGTG GTTGAGCCTG GTATGCTTGT CGAAGGACCA CCAGGACCAG | 1500 |
| CAGGACCTGC AGGTATTATG GGTCCTCCAG GTCTACAAGG CCCCACTGGA CCCCCTGGTG | 1560 |
| ACCCTGGCGA TAGGGGCCCC CCAGGACGTC CTGGCTTACC AGGGGCTGAT GGTCTACCTG | 1620 |
| GTCCTCCTGG TACTATGTTG ATGTTACCGT TCCGTTATGG TGGTGATGGT TCCAAAGGAC | 1680 |
| CAACCATCTC TGCTCAGGAA GCTCAGGCTC AAGCTATTCT TCAGCAGGCT CGGATTGCTC | 1740 |
| TGAGAGGCCC ACCTGGCCCA ATGGGTCTAA CTGGAAGACC AGGTCCTGTG GGGGGGCCTG | 1800 |
| GTTCATCTGG GGCCAAAGGT GAGAGTGGTG ATCCAGGTCC TCAGGGCCCT CGAGGCGTCC | 1860 |
| AGGGTCCCCC TGGTCCAACG GGAAAACCTG GAAAAAGGGG TCGTCCAGGT GCAGATGGAG | 1920 |
| GAAGAGGAAT GCCAGGAGAA CCTGGGGCAA AGGGAGATCG AGGGTTTGAT GGACTTCCGG | 1980 |
| GTCTGCCAGG TGACAAAGGT CACAGGGGTG AACGAGGTCC TCAAGGTCCT CCAGGTCCTC | 2040 |
| CTGGTGATGA TGGAATGAGG GGAGAAGATG GAGAAATTGG ACCAAGAGGT CTTCCAGGTG | 2100 |
| AAGCTGGCCC ACGAGGTTTG CTGGGTCCAA GGGGAACTCC AGGAGCTCCA GGGCAGCCTG | 2160 |
| GTATGGCAGG TGTAGATGGC CCCCCAGGAC CAAAAGGGAA CATGGGTCCC CAAGGGGAGC | 2220 |
| CTGGGCCTCC AGGTCAACAA GGGAATCCAG GACCTCAGGG TCTTCCTGGT CCACAAGGTC | 2280 |
| CAATTGGTCC TCCTGGTGAA AAAGGACCAC AAGGAAAACC AGGACTTGCT GGACTTCCTG | 2340 |
| GTGCTGATGG GCCTCCTGGT CATCCTGGGA AAGAAGGCCA GTCTGGAGAA AAGGGGGCTC | 2400 |
| TGGGTCCCCC TGGTCCACAA GGTCCTATTG GATNNCCGGG CCCCCGGGGA GTAAAGGGAG | 2460 |
| CAGATGGTGT CAGAGGTCTC AAGGGATCTA AAGGTGAAAA GGGTGAAGAT GGTTTTCCAG | 2520 |
| GATTCAAAGG TGACATGGGT CTAAAAGGTG ACAGAGGAGA AGTTGGTCAA ATTGGCCCAA | 2580 |
| GAGGGNAAGA TGGCCCTGAA GGACCCAAAG GTCGAGCAGG CCCAACTGGA GACCCAGGTC | 2640 |
| CTTCAGGTCA AGCAGGAGAA AAGGGAAAAC TTGGAGTTCC AGGATTACCA GGATATCCAG | 2700 |
| GAAGACAAGG TCCAAAGGGT TCCACTGGAT TCCCTGGGTT TCCAGGTGCC AATGGAGAGA | 2760 |
| AAGGTGCACG GGGAGTAGCT GGCAAACCAG GCCCTCGGGG TCAGCGTGGT CCAACGGGTC | 2820 |
| CTCGAGGTTC AAGAGGTGCA AGAGGTCCCA CTGGGAAACC TGGGCCAAAG GGCACTTCAG | 2880 |
| GTGGCGATGG CCCTCCTGGC CCTCCAGGTG AAAGAGGTCC TCAAGGACCT CAGGGTCCAG | 2940 |
| TTGGATTCCC TGGACCAAAA GGCCCTCCTG GACCACCAGG AAGGATGGGC TGCCCAGGAC | 3000 |
| ACCCTGGGCA ACGTGGGGAG ACTGGATTTC AAGGCAAGAC CGGCCCTCCT GGGCCAGGGG | 3060 |
| GAGTGGTTGG ACCACAGGGA CCAACCGGTG AGACTGGTCC AATAGGGGAA CGTGGGTATC | 3120 |
| CTGGTCCTCC TGGCCCTCCT GGTGAGCAAG GTCTTCCTGG TGCTGCAGGA AAAGAAGGTG | 3180 |
| CAAAGGGTGA TCCAGGTCCT CAAGGTATCT CAGGGAAAGA TGGACCAGCA GGATTACGTG | 3240 |
| GTTTCCCAGG GGAAAGAGGT CTTCCTGGAG CTCAGGGTGC ACCTGGACTG AAAGGAGGGG | 3300 |
| AAGGTCCCCA GGGCCCACCA GGTCCAGTTG GCTCACCAGG AGAACGTGGG TCAGCAGGTA | 3360 |
| CAGCTGGCCC AATTGGTTTA CGAGGGCGCC CGGGACCTCA GGGTCCTCCT GGTCCAGCTG | 3420 |
| GAGAGAAAGG TGCTCCTGGA GAAAAAGGTC CCCAAGGGCC TGCAGGGAGA GATGGAGTTC | 3480 |
| AAGGTCCTGT TGGTCTCCCA GGGCCAGCTG GTCCTGCCGG CTCCCCTGGG GAAGACGGAG | 3540 |
| ACAAGGGTGA AATTGGTGAG CCGGGACAAA AAGGCAGCAA GGGTGGCAAG GGAGAAAATG | 3600 |
| GCCCTCCCGG TCCCCCAGGT CTTCAAGGAC CAGTTGGTGC CCCTGGAATT GCTGGAGGTG | 3660 |
| ATGGTGAACC AGGTCCTAGA GGACAGCAGG GGATGTTTGG GCAAAAAGGT GATGAGGGTG | 3720 |
| CCAGAGGCTT CCCTGGACCT CCTGGTCCAA TAGGTCTTCA GGGTCTGCCA GGCCCACCTG | 3780 |
| GTGAAAAAGG TGAAAATGGG GATGTTGGTC CATGGGGGCC ACCTGGTCCT CCAGGCCCAA | 3840 |
| GAGGCCCTCA AGGTCCCAAT GGAGCTGATG GACCACAAGG ACCCCCAGGT TCTGTTGGTT | 3900 |
| CAGTTGGTGG TGTTGGAGAA AAGGGTGAAC CTGGAGAAGC AGGAAACCCA GGGCCTCCTG | 3960 |
| GGGAAGCAGG TGTAGGCGGT CCCAAAGGAG AAAGAGGAGA GAAAGGGGAA GCTGGTCCAC | 4020 |
| CTGGAGCTGC TGGACCTCCA GGTGCCAAGG GGCCGCCAGG TGATGATGGC CCTAAGGGTA | 4080 |
| ACCCGGGTCC TGTTGGTTTT CCTGGAGATC CTGGTCCTCC TGGGGAACTT GGCCCTGCAG | 4140 |
| GTCAAGATGG TGTTGGTGGT GACAAGGGTG AAGATGGAGA TCCTGGTCAA CCGGGTCCTC | 4200 |
| CTGGCCCATC TGGTGAGGCT GGCCCACCAG GTCCTCCTGG AAAACGAGGT CCTCCTGGAG | 4260 |
| CTGCAGGTGC AGAGGGAAGA CAAGGTGAAA AAGGTGCTAA GGGGGAAGCA GGTGCAGAAG | 4320 |
| GTCCTCCTGG AAAAACCGGC CCAGTCGGTC CTCAGGGACC TGCAGGAAAG CCTGGTCCAG | 4380 |
| AAGGTCTTCG GGGCATCCCT GGTCCTGTGG GAGAACAAGG TCTCCCTGGA GCTGCAGGCC | 4440 |
| AAGATGGACC ACCTGGTCCT ATGGGACCTC CTGGCTTACC TGGTCTCAAA GGTGACCCTG | 4500 |
| GCTCGAAGGG TGAAAAGGGA CATCCTGGTT TAATTGGCCT GATTGGTCCT CCAGGAGAAC | 4560 |
| AAGGGGAAAA AGGTGACCGA GGGCTCCCTG GAACTCAAGG ATCTCCAGGA GCAAAAGGGG | 4620 |
| ATGGGGGAAT TCCTGGTCCT GCTGGTCCCT TAGGTCCACC TGGTCCTCCA GGCTTACCAG | 4680 |
| GTCCTCAAGG CCCAAAGGGT AACAAAGGCT CTACTGGACC CGCTGGCCAG AAAGGTGACA | 4740 |
| GTGGTCTTCC AGGGCCTCCT GGGCCTCCAG GTCCACCTGG TGAAGTCATT CAGCCTTTAC | 4800 |
| CAATCTTGTC CTCCAAAAAA ACGAGAAGAC ATACTGAAGG CATGCAAGCA GATGCAGATG | 4860 |
| ATAATATTCT TGATTACTCG GATGGAATGG AAGAAATATT TGGTTCCCTC AATTCCCTGA | 4920 |
| AACAAGACAT CGAGCATATG AAATTTCCAA TGGGTACTCA GACCAATCCA GCCCGAACTT | 4980 |
| GTAAAGACCT GCAACTCAGC CATCCTGACT TCCCAGATGG TGAATATTGG ATTGATCCTA | 5040 |
| ACCAAGGTTG CTCAGGAGAT TCCTTCAAAG TTTACTGTAA TTTCACATCT GGTGGTGAGA | 5100 |
| CTTGCATTTA TCCAGACAAA AAATCTGAGG GAGTAAGAAT TTCATCATGG CCAAAGGAGA | 5160 |
| AACCAGGAAG TTGGTTTAGT GAATTTAAGA GGGGAAAACT GCTITCATAC TTAGATGTTG | 5220 |
| AAGGAAATTC CATCAATATG GTGCAAATGA CATfCCTGAA ACTTCTGACT GCCTCTGCTC | 5280 |
| GGCAAAATTT CACCTACCAC TGTCATCAGT CAGCAGCCTG GTATGATGTG TCATCAGGAA | 5340 |
| GTTATGACAA AGCACTTCGC TTCCTGGGAT CAAATGATGA GGAGATGTCC TATGACAATA | 5400 |
| ATCCTTTTAT CAAAACACTG TATGATGGTT GTACGTCCAG AAAAGGCTAT GAAAAAACTG | 5460 |
| TCATTGAAAT CAATACACCA AAAATTGATC AAGTACCTAT TGTTGATGTC ATGATCAGTG | 5520 |
| ACTTTGGTGA TCAGAATCAG AAGTTCGGAT TTGAAGTTGG TCCTGTTTGT TTTCTTGGCT | 5580 |
| AAGATTAAGA CAAAGAACAT ATCAAATCAA CAGAAAATGT ACCTTGGTGC CACCAACCCA | 5640 |
| TITTGTGCCA CATGCAAGTT TTGAATAAGG ATGTATGGAA AACAACGCTG CATATACAGG | 5700 |
| TACCATTTAG GAAATACCGA TGCCTTTGTG GGGGCAGAAT CACAGACAAA AGCTTTGAAA | 5760 |
| ATCATAAAGA TATAAGTTGG TGTGGCTAAG ATGGAAACAG GGCTGATTCT TGATTCCCAA | 5820 |
| TTCTCAACTC TCCTTTTCCT ATTTGAATTT CTTTGGTGCT GTAGAAAACA AAAAAAGAAA | 5880 |
| AATATATATT CATAAAAAAT ATGGTGCTCA TTCTCATCCA TCCAGGATGT ACTAAAACAG | 5940 |
| TGTGTITAAT AAAYTGTAAT TATTTTGTGT ACAGTTCTAT ACTGTTATCT GTGTCCATTT | 6000 |
| CCAAAACTTG CACGTGTCCC TGAATTCCGC TGACTCTAAT TTATGAGGAT GCCGAACTCT | 6060 |
| GATGGCAATA ATATATGTAT TATGAAAATG AAGTTATGAT TTCCGATGAC CCTAAGTCCC | 6120 |
| TTTCTTTGGT TAATGATGAA ATTCCTTTGT GTGTGTTT | |

Seq ID NO: 122 Protein sequence:
Protein Accession #: NP_001845

| | |
|---|---|
| 1 11 21 31 41 51 | |
| \| \| \| \| \| \| | |
| MEPWSSRWKT KRWLWDFTVT TLALTFLFQA RBVRGAAPVD VLKALDFHNS PEGISKTTGF | 60 |
| CTNRKNSKGS DTAYRVSKQA QLSAPTKQLF PGGTFPEDFS ILFTVKPKKG IQSFLLSIYN | 120 |
| EHGIQQIGVE VGRSPVFLFE DHTGKPAPED YPLFRTVNIA DGKWHRVAIS VEKKTVTMIV | 180 |
| DCKKKTTKPL DRSERAIVDT NGITVFGTRI LDEEVFEGDI QQFLITGDPK AAYDYCEHYS | 240 |
| PDCDSSAPKA AQAQEPQIDE YAPEDIIEYD YEYGEAEYKE AESVTEGPTV TEETIAQTEA | 300 |
| NIVDDFQEYN YGTMESYQTE APRHVSGTNE PNPVEEIFTE EYLTGEDYDS QRKNSEDTLY | 360 |
| ENKEIDGRDS DLLVDGDLGE YDFYEYKEYE DKPTSPPNEE FGPGVPAETD ITETSINGHG | 420 |
| AYGEKGQKGE PAVVEPGMLV EGPPGPAGPA GIMGPPGLQG PTGPPGDPGD RGPPGRPGLP | 480 |
| GADGLPGPPG TMLMLPFRYG GDGSKGPTIS AQEAQAQAIL QQARIALRGP PGPMGLTGRP | 540 |
| GPVGGPGSSG AKGESGDPGP QGPRGVQGPP GPTGKPGKRG RPGADGGRGM PGEPGAKGDR | 600 |
| GFDGLPGLPG DKGHRGERGP QGPPGPPGDD GMRGEDGEIG PRGLPGEAGP RGLLGPRGTP | 660 |
| GAPGQPGMAG VDGPPGPKGN MGPQGEPGPP GQQGNPGPQG LPGPQGPIGP PGEKGPQGKP | 720 |
| GLAGLPGADG PPGHPGKEGQ SGEKGALGPP GPQGPIGXPG PRGVKGADGV RGLKGSKGEK | 780 |
| GEDGFPGFKG DMGLKGDRGE VGQIGPRGXD GPEGPKGRAG PTGDPGPSGQ AGEKGKLGVP | 840 |
| GLPGYPGRQG PKGSTGFPGF PGANGEKGAR GVAGKPGPRG QRGPTGPRGS RGARGPTGKP | 900 |
| GPKGTSGGDG PPGPPGERGP QGPQGPVGFP GPKGPPGPPG RMGCFGHPGQ RGETGFQGKT | 960 |
| GPPGPGGVVG PQGPTGETGP IGERGYPGPP GPPGEQGLFG AAGKEGAKGD PGPQGISGKD | 1020 |
| GPAGLRGFPG ERGLPGAQGA PGLKGGEGPQ GPPGPVGSPG ERGSAGTAGP IGLRGRTGPQ | 1080 |
| GPPGPAGEKG APGEKGPQGP AGRDGVQGPV GLPGPAGPAG SPGEDGDKGE IGEPGQKGSK | 1140 |
| GGKGENGPPG PPGLQGPVGA PGIAGGDGEP GPRGQQGMFG QKGDEGARGF PGPPGPIGLQ | 1200 |
| GLPGFPGEKG ENGDVGPWGP PGPPGPRGPQ GPNGADGPQG PPGSVGSVGG VGEKGEPGEA | 1260 |
| GNPGPPGEAG VGGPKGERGE KGEAGPPGAA GPPGAKGPPG DDGPKGNPGP VGFPGDPGPP | 1320 |
| GELGPAGQDG VGGDKGEDGD PGQPGPPGPS GEAGPPGPPG KRGPPGAAGA EGRQGEKGAK | 1380 |
| GEAGAEGPDG KTGPVGPQGP AGKPGPEGLR GIPGPVGEQG LPGAAGQDGP PGPMGPPGLP | 1440 |
| GLKGDPGSKG EKGHPGLIGL IGPPGEQGEK GDRGLPGTQG SPGAKGDGGI PGPAGPLGPP | 1500 |
| GPPGLPGPQG PKGNXGSTGP AGQKGDSGLP GPPGPPGPPG EVIQPLPILS SKKTRRHTEG | 1560 |
| MQADADDNIL DYSDGMEEIE GSLNSLKQDI EHMKFPMGTQ TNPARTCKDL QLSHPDFPDG | 1620 |
| EYWIDPNQGC SGDSFKVYCN FTSGGETCIY PDKKSEGVRI SSWPKEKPGS WFSEFKRGKL | 1680 |
| LSYLDVEGNS INMVQMTFLK LLTASARQNP TYHCHQSAAW YDVSSGSYDK ALRFLGSNDE | 1740 |
| EMSYDNNPFI KTLYDGCTSR KGYEKTVIEI NTPKIDQVPI VDVMISDFGD QNQKFGFEVG | 1800 |
| PVCFLG | |

Seq ID NO: 123 DNA sequence
Nucleic Acid Accession #: NM_015886
Coding sequence: 485-1261 (underlined sequences correspond to start
and stop codons)

| | |
|---|---|
| 1 11 21 31 41 51 | |
| \| \| \| \| \| \| | |
| GAATTCCCCC CCCCCCCCCC TCACTTGGTG TGTCTATATG TCTGGCAGAC ATTATCAGCA | 60 |
| CATTCTCTGT TGTTACCTGT GATTCATTTT TTCTTCACTC TCCAGGTGAA TTTCAATTGC | 120 |
| TGAAAATTTC CCACTGAAAA TATGCAGTAA TATATTTTGT GGTTCAGACA TTTGGGGCAA | 180 |
| ATGGTTCACA TTCATTTTAG GGTTAGTGGT CATGCTGTTT ATTTTTCTCT GCTATACAAA | 240 |
| GTTCCTCTTA GGGGTCTGCC TCATGACACT AAAAAATGAA TAGAGATTCT ACTGTAGGTT | 300 |
| ATCTCCTAGG CTTGAGTTCA ACATTTGTTT GGATITTTGA AGAAAGTGAA ATCAAGCAAT | 360 |
| GCTCCCAAAT GATGTCTTTG TAAATTCATA CCCTCTGGCC CTATTTTTTT TCATAGACCC | 420 |
| TAACTCTACC TTTCTGCTIT AAAGCAAAGT AAACTCGGTG GCCTCTTCTT CTCCACCCCT | 480 |
| CAAAATGATA GCAATCTCTG CCGTCAGCAG TGCACTCCTG TTCTCCCTTC TCTGTGAAGC | 540 |
| AAGTACCGTC GTCCTACTCA ATTCCACTGA CTCATCCCCG CCAACCAATA ATTTCACTGA | 600 |
| TATTGAAGCA GCTCTGAAAG CACAATTAGA TTCAGCGGAT ATCCCCAAAG CCAGGCGGAA | 660 |
| GCGCTACATT TCGCAGAATG ACATGATCGC CATTCTTGAT TATCATAATC AAGTTCGGGG | 720 |
| CAAAGTGTTC CCACCGGCAG CAAATATGGA ATATATGGTT TGGGATGAAA ATCTTGCAAA | 780 |
| ATCGGCAGAG GCTTGGGCGG CTACTTGCAT TTGGGACCAT GGACCTTCTT ACTTACTGAG | 840 |
| ATTTTTGGGC CAAAATCTAT CTGTACGCAC TGGAAGATAT CGCTCTATTC TCCAGTTGGT | 900 |
| CAAGCCATGG TATGATGAAG TGAAAGATTA TGCTTTTCCA TATCCCCAGG ATTGCAACCC | 960 |
| CAGATGTCCT ATGAGATGTT TTGGTCCCAT GTGCACACAT TATACGCAGA TGGTTTGGGC | 1020 |
| CACTTCCAAT CGGATAGGAT GCGCAATTCA TGCTTGCCAA AACATGAATG TTTGGGGATC | 1080 |
| TGTGTGGCGA CGTGCAGTTT ACTTGGTATG CAACTATGCC CCAAAGGGCA ATTGGATTGG | 1140 |
| AGAAGCACCA TATAAAGTAG GGGTACCATG TTCATCTTGT CCTCCAAGTT ATGGGGGATC | 1200 |
| TTGTACTGAC AATCTGTGTT TTCCAGGAGT TACGTCAAAC TACCTGTACT GGTTTAAATA | 1260 |
| AG1TTACCTT TTCCTCCAGG AAATATAATG ATTTCTGGGA ACATGGGCAT GTATATATAT | 1320 |
| ATATGGAGAG AGAATTTTGC ACATATTATA CATATTTTGT GCTAATCTTG TTTTCCTCTT | 1380 |
| AGTATTCCTT TGTATAAATT AGTGTTTGTC TAGCATGTTT GTTTAATCCT TTGGGAATTC | |

Seq ID NO: 124 Protein sequence:
Protein Accession #: NP_056970.1

| | |
|---|---|
| 1 11 21 31 41 51 | |
| \| \| \| \| \| \| | |
| MIAISAVSSA LLFSLLCEAS TVVLLNSTDS SPPTNNETDI EAALKAQLDS ADIPKARRKR | 60 |
| YISQNDMIAI LDYHNQVRGK VFPPAANMEY MVWDENLAKS AEAWAATCIW DHGPSYLLRF | 120 |
| LGQNLSVRTG RYRSILQLVK PWYDEVKDYA FPYPQDCNPR CPMRCFGPMC THYTQMVWAT | 180 |
| SNRIGCAIHA CQNMNVWGSV WRRAVYLVCN YAPKGNWIGE APYKVGVPCS SCPPSYGGSC | 240 |
| TDNLCFPGVT SNYLYWFK | |

Seq ID NO: 125 DNA sequence
Nucleic Acid Accession #: NM_001793
Coding sequence: 54-2543 (underlined sequences correspond to start and
stop codons)

| | |
|---|---|
| 1 11 21 31 41 51 | |
| \| \| \| \| \| \| | |
| GCGGAACACC GGCCCGCCGT CGCGGCAGCT GCTTCACCCC TCTCTCTGCA GCCATGGGGC | 60 |
| TCCCTCGTGG ACCTCTCGCG TCTCTCCTCC TTCTCCAGGT TTGCTGGCTG CAGTGCGCGG | 120 |
| CCTCCGAGCC TTGCCGGGCG GTCTTCAGGG AGGCTTAAGT GACCTTGGAG GCGGGAGGCG | 180 |
| CGGAGCAGGA GCCCGGCCAG GCGCTGGGGA AAGTATTCAT GGGCTGCCCT GGGCAAGAGC | 240 |
| CAGCTCTGTT TAGCACTGAT AATGATGACT TCACTGTGCG GAATGGCGAG ACAGTCCAGG | 300 |
| AAAGAAGGTC ACTGAAGGAA AGGAATCCAT TGAAGATCTT CCCATCCAAA CGTATCTTAC | 360 |
| GAAGACACAA GAGAGATTGG GTGGTTGCTC CAATATCTGT CCCTGAAAAT GGCAAGGGTC | 420 |
| CCTTCCCCCA GAGACTGAAT CAGCTCAAGT CTAATAAAGA TAGAGACACC AAGATTTTCT | 480 |
| ACAGCATCAC GGGGCCGGGG GCAGACAGCC CCCCTGAGGG TGTCTTCGCT GTAGAGAAGG | 540 |
| AGACAGTCTG GTTGTTGTTG AATAAGCCAC TGGACCGGGA GGAGATIGCC AAGTATGAGC | 600 |
| TCTTTGGCCA CGCTGTGTCA GAGAATGGTG CCTCAGTGGA GGACCCCATG AACATCTCCA | 660 |
| TCATCGTGAC CGACCAGAAT GACCACAAGC CCAAGTTTAC CCAGGACACC TTCCGAGGGA | 720 |
| GTGTCTTAGA GGGAGTCCTA CCAGGTACTT CTGTGATGCA GGTGACAGCC ACAGATGAGG | 780 |
| ATGATGCCAT CTACACCTAC AATGGGGTGG TTGCTTACTC CATCCATAGC CAAGAACCAA | 840 |
| AGGACCCACA CGACCTCATG TTCACAATTC ACCGGAGCAC AGGCACCATC AGCGTCATCT | 900 |
| CCAGTGGCCT GGACCGGGAA AAAGTCCCTG AGTACACACT GACCATCCAG GCCACAGACA | 960 |
| TGGATGGGGA CGGCTCCACC ACCACGGCAG TGGCAGTAGT GGAGATCCTT GATGCCAATG | 1020 |
| ACAATGCTCC CATGTTTGAC CCCCAGAAGT ACGAGGCCCA TGTGCCTGAG AATGCAGTGG | 1080 |
| GCCATGAGGT GCAGAGGCTG ACGGTCACTG ATCTGGACGC CCCCAACTCA CCAGCGTGGC | 1140 |
| GTGCCACCTA CCTTATCATG GGCGGTGACG ACGGGGACCA TTTTACCATC ACCACCCACC | 1200 |
| CTGAGAGCAA CCAGGGCATC CTGACAACCA GGAAGGGTTT GGATTTTGAG GCCAAAAACC | 1260 |
| AGCACACCCT GTACGTTGAA GTGACCAACG AGGCCCCTTT TGTGCTGAAG CTCCCAACCT | 1320 |
| CCACAGCCAC CATAGTGGTC CACGTGGAGG ATGTGAATGA GGCACCTGTG TTTGTCCCAC | 1380 |
| CCTCCAAAGT CGTTGAGGTC CAGGAGGGCA TCCCCACTGG GGAGCCTGTG TGTGTCTACA | 1440 |
| CTGCAGAAGA CCCTGACAAG GAGAATCAAA AGATCAGCTA CCGCATCCTG AGAGACCCAG | 1500 |
| CAGGGTGGCT AGCCATGGAC CCAGACAGTG GGCAGGTCAC AGCTGTGGGC ACCCTCGACC | 1560 |
| GTGAGGATGA GCAGTTTGTG AGGAACAACA TCTATGAAGT CATGGTCTTG GCCATGGACA | 1620 |
| ATGGAAGCCC TCCCACCACT GGCACGGGAA CCCTTCTGCT AACACTGATT GATGTCAACG | 1680 |
| ACCATGGCCC AGTCCCTGAG CCCCGTCAGA TCACCATCTG CAACCAAAGC CCTGTGCGCC | 1740 |
| ACGTGCTGAA CATCACGGAC AAGGACCTGT CTCCCCACAC CTCCCCTITC CAGGCCCAGC | 1800 |
| TCACAGATGA CTCAGACATC TACTGGACGG CAGAGGTCAA CGAGGAAGGT GACACAGTGG | 1860 |
| TCTTGTCCCT GAAGAAGTTC CTGAAGCAGG ATACATATGA CGTGCACCTT TCTCTGTCTG | 1920 |
| ACCATGGCAA CAAAGAGCAG CTGACGGTGA TCAGGGCCAC TGTGTGCGAC TGCCATGGCC | 1980 |
| ATGTCGAAAC CTGCCCTGGA CCCTGGAAAG GAGGTTTCAT CCTCCCTGTG CTGGGGGCTG | 2040 |
| TCCTGGCTCT GCTGTTCCTC CTGCTGGTGC TGCTTTTGTT GGTGAGAAAG AATCGGAAGA | 2100 |
| TCAAGGAGCC CCTCCTACTC CCAGAAGATG ACACCCGTGA CAACGTCTFC TACTATGGCG | 2160 |
| AAGAGGGGTG TGGCGAAGAG QACCAGGACT ATGACATCAC CCAGCTCCAC CGAGGTCTGG | 2220 |
| AGGCCAGGCC GGAGGTGGTT CTCCGCAATG ACGTGGCACC AACCATCATC CCGACACCCA | 2280 |
| TGTACCGTCC TAGGCCAGCC AACCCAGATG AAATCGGCAA CTTTATAATT GAGAACCTGA | 2340 |
| AGGCGGCTAA CACAGACCCC ACAGCCCCGC CCTACGACAC CCTCTTGGTG TTCGACTATG | 2400 |
| AGGGCAGCGG CTCCGACGCC GCGTCCCTGA GCTCCCTCAC CTCCTCCGCC TCCGACCAAG | 2460 |
| ACCAAGATTA CGATTATCTG AACGAGTGGG GCAGCCGCTT CAAGAAGCTG GCAGACATGT | 2520 |
| ACGGTGGCGG GGAGGAGGAC TAGGCGGCCT GCCTGCAGGG CTGGGGACCA AACGTCAGGC | 2580 |
| CACAGAGCAT CTCCAAGGGG TCTCAGTTCC CCCTTCAGCT GAGGACTTCG GAGCTTGTCA | 2640 |
| GGAAGTGGCC GTAGCAACTT GGCGGAGACA GGCTATGAGT CTGACGTTAG AGTGGTTGCT | 2700 |
| TCCTTAGCCT TTCAGGATGG AGGAATGTGG GCAGTTTGAC TTCAGCACTG AAAACCTCTC | 2760 |
| CACCTGGGCC AGGGTTGCCT CAGAGGCCAA GTTTCCAGAA GCCTCTTACC TGCCGTAAAA | 2820 |
| TGCTCAACCC TGTGTCCTGG GCCTGGGCCT GCTGTGACTG ACCTACAGTG GACTTTCTCT | 2880 |
| CTGGAATGGA ACCTTCTTAG GCCTCCTGGT GCAACTTAAT TTTTTTTTTT AATGCTATCT | 2940 |
| TCAAAACGTT AGAGAAAGTT CTTCAAAAGT GCAGCCCAGA GCTGCTGGGC CCACTGGCCG | 3000 |
| TCCTGCATTT CTGGTTTCCA GACCCCAATG CCTCCCATTC GGATGGATCT CTGCGTTTTT | 3060 |
| ATACTGAGTG TGCCTAGGTT GCCCCTTATT TTTTATTTTC CCTGTTGCGT TGCTATAGAT | 3120 |
| GAAGGGTGAG GACAATCGTG TATATGTACT AGAACTTTTT TATTAAAGAA A | |

Seq ID NO: 126 Protein sequence:
Protein Accession #: NP_001784

| | |
|---|---|
| 1 11 21 31 41 51 | |
| \| \| \| \| \| \| | |
| MGLPRGPLAS LLLLQVCWLQ CAASEPCRAV FREAEVTLEA GGAEQEPGQA LGKVFMGCPG | 60 |
| QEPALFSTDN DDFTVRNGET VQERRSLKER NPLKIFPSKR ILRRHKRDWV VAPISVPENG | 120 |
| KGPFPQRLNQ LKSNKDRDTK IFYSITGPGA DSPPEGVFAV EKETGWLLLN KPLDREEIAK | 180 |
| YELFGHAVSE NGASVEDPMN ISIIVTDQND HKPCFTQDTF RGSVLEGVLP GTSVMQVTAT | 240 |
| DEDDATYTYN GVVAYSIHSQ EPKDPHDLMF TIHRSTGTIS VISSGLDRBK VPEYTLTIQA | 300 |
| TDMDGDGSTT TAVAVVEILD ANDNAPMFDP QKYEAHVPEN AVGHEVQRLT VTDLDAPNSP | 360 |
| AWRATYLIMG GDDGDHFTIT THPESNQGIL TTRXGLDFEA KNQHTLYVEV TNEAPFVLKL | 420 |
| PTSTATIVVH VEDVNEAPVF VPPSKVVEVQ EGIPTGEPVC VYTAEDPDKE NQKISYRTLR | 480 |
| DPAGWLAMDP DSGQVTAVGT LDREDEQFVR NNTYEVMVLA MDNGSPPTTG TGTLLLTLID | 540 |
| VNDHGPVPEP RQITICNQSP VRHVLNITDK DLSPHTSPFQ AQLTDDSDIY WTAEVNEEGD | 600 |
| TVVLSLKKPL KQDTYDVHLS LSDHGNKEQL TVIRATVCDC HGHVETCPGP WKGGFILPVL | 660 |
| GAVLALLFLL LVLLLLVRKK RKIKEPLLLP EDDTRDNVFY YGEEGGGEED QDYDITQLHR | 720 |
| GLEARPEVVL RNDVAPTIIP TPMYRPRPAN PDEIGNFIIE NLKAANTDPT APPYDTLLVF | 780 |
| DYEGSGSDAA SLSSLTSSAS DQDQDYDYLN EWGSRFCKLA DMYGGGEDD | |

Seq ID NO: 127 DNA sequence
Nucleic Acid Accession #: NM_003256.1
Coding sequence: 60-734 (underlined sequences correspond to start and
stop codons)

| | |
|---|---|
| 1 11 21 31 41 51 | |
| \| \| \| \| \| \| | |
| CCTGCTGGGG CCGTCCAGTC CCCCAGACCT CACAGGCTCA GTCGCGGATC TGCAGTGTCA | 60 |
| TGCCTGGGAG CCCTCGGCCC GCGCCAAGCT GGGTGCTGTT GCTGCGGCTG CTGGCGTTGC | 120 |
| TGCGGCCCCC GGGGCTGGGT GAGGCATGCA GCTGCGCCCC GGCGCACCCT CAGCAGCACA | 180 |
| TCTGCCACTC GGCAC1TGTG ATTCGGGCCA AAATCTCCAG TGAGAAGGTA GTTCCGGCCA | 240 |
| GTGCAGACCC TGCTGACACT GAAAAAATGC TCCGGTATGA AATCAAACAG ATAAAGATGT | 300 |
| TCAAAGGGTT TGAGAAAGTC AAGGATGTTC AGTATATCTA TACGCCTTTT GACTCTTCCC | 360 |
| TCTGTGGTGT GAAACTAGAA GCCAACAGCC AGAAGCAGTA TCTCTTGACT GGTCAGGTCC | 420 |
| TCAGTGATGG AAAAGTCTTC ATCCATCTGT GCAACTACAT CGAGCCCTGG GAGGACCTGT | 480 |
| CCTTGGTGCA GAGGGAAAGT CTGAATCATC ACTACCATCT GAACTGTGGC TGCCAAATCA | 540 |
| CCACCTGCTA CACAGTACCC TGTACCATCT CGGCCCCTAA CGAGTGCCTC TGGACAGACT | 600 |
| GGCTGTTGGA ACGAAAGCTC TATGGTTACC AGGCTCAGCA TTATGTCTGT ATGAAGCATG | 660 |
| TTGACGGCAC CTGCAGCTGG TACCGGGGCC ACCTGCCTCT CAGGAAGGAG TTTGTTGACA | 720 |
| TCGTTCAGCC CTAGTAGGGA CCAGTGACCA TCACATCCCT TCAAGAGTCC TGAAGATCAA | 780 |
| GCCAGTTCTC CTTCCCTGCA GAGCTTTGGC CATTACCACC TGACCTCTTG CTGCCAGCTA | 840 |
| ATAAGAAGTG CCAAGTGGAC ATTCTGGCCA CTGTCAAGGC AGGGAAGGGG CCATGACTTT | 900 |
| TCTGCCCTGC CCTCAGCCTG TTGCCCTGCC TCCCAAACCC CATTAGTCTA GCCTTGTAGC | 960 |
| TGTTACTGCA AGTGTTTCTT CTGGCTTAGT CTGTTTTCTA AAGCCAGGAC TATTCCCTTT | 1020 |
| CCTCCCCAGG AATATGTGTT TTCCTTTGTC TTAATCGATC TGGTAGGGGA GAAATGGCGA | 1080 |
| ATGTCATACA CATGAGATGG TATATCCTTG CGATGTACAG AATCAGAAGG TGGTTTGACA | 1140 |
| GCATCATAAA CAGGCTGACT GGCAGGAATG AAAAAAAAAA AAAAAAAAA | |

Seq ID NO: 128 Protein sequence:
Protein Accession #: NP_003247.1

| | |
|---|---|
| 1 11 21 31 41 51 | |
| \| \| \| \| \| \| | |
| MPGSPRPAPS WVLLLRLLAL LRPPGLGEAC SCAPAHPQQH ICHSALVIRA KISSEKVVPA | 60 |
| SADPADTEKM LRYEIKQIKM FKGFEKVKDV QYIYTPFDSS LCGVKLEANS QKQYLLTGQV | 120 |
| LSDGKVFIIL CNYIEPWEDL SLVQRESLNH HYILNCGCQI TTCYTVPCTI SAPNECLWTD | 180 |
| WLLERXLYGY QAQHYVCMKH VDGTCSWYRH HLPLRKEFVD IVQP | |

Seq ID NO: 129 DNA sequence
Nucleic Acid Accession #: NM_007207.2
Coding sequence: 143-1591 (underlined sequences correspond to start
and stop codons)

| | |
|---|---|
| 1 11 21 31 41 51 | |
| \| \| \| \| \| \| | |
| CCACGCGTCC GCAATGAAGC CGAGTGAATG GGGGCTGAAT GTGCGAGTCC ATAGCTGAAG | 60 |
| AGGAGCGCCA GATGGTGGAG GAATACACTT ATTTATGAAA CTGTCTTGAG TTCTTCTTGA | 120 |
| ATTGCCAGTT TTCAGCCTCC TCATGCCTCC GTCTCCTTTA GACGACAGGG TAGTAGTGGC | 180 |
| ACTATCTAGG CCCGTCCGAC CTCAGGATCT CAACCTTTGT TTAGACTCTA GTTACCTTGG | 240 |
| CTCTGCCAAC CCAGGCAGTA ACAGCCACCC TCCTGTCATC GCCACCACCG TTGTGTCCCT | 300 |
| CAAGGCTGCG AATCTGACGT ATATGCCCTC ATCCAGCGGC TCTGCCCGCT CGCTGAATTG | 360 |
| TGGATGCAGC AGTGCCAGCT GCTGCACTGT GGCAACCTAC GACAAGGACA ATCAGGCCCA | 420 |
| AACCCAAGCC ATTGCCGCTG GCACCACCAC CACTGCCATC GGAACCTCTA CCACCTGCCC | 480 |
| TGCTAACCAG ATGGTCAACA ATAATGAGAA TACAGGCTCT CTAAGTCCAT CAAGTGGGGT | 540 |
| GGGCAGCCCT GTGTCAGGGA CCCCCAAGCA GCTAGCCAGC ATCAAAATAA TCTACCCCAA | 600 |
| TGACTTGGCA AAGAAGATGA CCAAATGCAG CAAGAGTCAC CTGCCGAGTC AGGGCCCTGT | 660 |
| CATCATTGAC TGCAGGCCCT TCATGGAGTA CAACAAGAGT CACATCCAAG GAGCTGTCCA | 720 |
| CATTAACTGT GCCGATAAGA TCAGCCGGCG GAGACTGCAG CAGGGCAAGA TCACTGTCCT | 780 |
| ATACTTGATT TCCTGTAGGG AAGGCAAGGA CTCTTTCAAG AGGATCTTTT CCAAAGAAAT | 840 |
| TATAGTTTAT GATGAGAATA CCAATGAACC AAGCCGAGTG ATGCCCTCCC AGCCACTTCA | 900 |
| CATAGTCCTC GAGTCCCTGA AGAGAGAAGG CAAAGAACCT CTGGTGTTGA AAGGTGGACT | 960 |
| TAGTAGTTTT AAGCAGAACC ATGAAAACCT CTGTGACAAC TCCCTCCAGC TCCAAGAGTG | 1020 |
| CCGGGAGGTG GGGGGCGGCG CATCCGCGGC CTCGAGCTTG CTACCTCAGC CCATCCCCAC | 1080 |
| CACCCCTGAC ATCGAGAACG CTGAGCTCAC CCCCATCTTG CCCTTCCTGT TCCTTGGCAA | 1140 |
| TGAGCAGGAT GCTCAGGACC TGGACACCAT GCAGCGGCTG AACATCGGCT ACGTCATCAA | 1200 |
| CGTCACCACT CATCTTCCCC TCTACCACTA TGAGAAAGGC CTGTTCAACT ACAAGCGGCT | 1260 |
| GCCAGCCACT GACAGCAACA AGCAGAACCT GCGGCAGTAC TTTGAAGAGT CTTTTGAGTT | 1320 |
| CATTGAGGAA GCTCACCAGT GTGGGAAGGG GCTTCTCATC CACTGCCAGG CTGGGGTGTC | 1380 |
| CCGCTCCGCC ACCATCGTCA TCGCTTACTT GATGAAGCAC ACTCGGATGA CCATGACTGA | 1440 |
| TGCTTATAAA TTTGTCAAAG GCAAACGACC AATTATCTCC CCAAACCTTA ACTTCATGGG | 1500 |
| GCAGTTGCTA GAGTfCGAGG AAGACCTAAA CAACGGTGTG ACACCGAGAA TCCTTACACC | 1560 |
| AAAGCTGATG GGCGTGGAGA CGGTTGTGTG ACAATGGTCT GGATGGAAAG GATTGCTGCT | 1620 |
| CTCCATTAGG AGACAATGAG GAAGGAGGAT GGATTCTGGT TTTTTTTCTT TCTTTTTTTT | 1680 |
| TTGTAGTTGG GAGTAAAGTT TGTGAATGTA AACAAACTTG GTTAAACACT TTATTTTTAA | 1740 |
| CAAGTGTAAG AAGACTATAC TTTTGATGCC ATTGAGATTC ACCTTCCACA AACTGGCCAA | 1800 |
| ATTAAGGAGG TTAAAGAAGT AATTTTTTTT AAGCCCAACC ATTAAAAATT TAATACAACT | 1860 |
| TGGTTTCTCC CCCTTTTTCC TTTAAAGCTA NTTTGTAAAA GTTTATGAG | |

Seq ID NO: 130 Protein sequence:
Protein Accession #: NP_009138.1

| | |
|---|---|
| 1 11 21 31 41 51 | |
| \| \| \| \| \| \| | |
| MPPSPLDDRV VVALSRPVRP QDLNLCLDSS YLGSANPGSN SHPPVIATTV VSLKAANLTY | 60 |
| MPSSSGSARS LNCGCSSASC CTVATYDKDN QAQTQAIAAG TTTTAIGTST TCPANQMVNN | 120 |
| NENTGSLSPS SGVGSPVSGT PKQLASIKII YPNDLAKKMT KCSKSHLPSQ GPVIIDCRPF | 180 |
| MEYNKSHIQG AVHINCADKI SRRRLQQGKI TVLDLISCRE GKDSFKRIFS KEIIVYDENT | 240 |
| NBPSRVMPSQ PLHIVLESLK REGKBPLVLK GGLSSFKQNH ENLCDNSLQL QECREVGGGA | 300 |
| SAASSLLPQP IPTTPDIENA ELTPILPELF LGNEQDAQDL DTMQRLNIGY VINVTTHLPL | 360 |
| YHYEKGLFNY KRLPATDSNK QNLRQYFEEA FEFIEEAHQC GKGLLIHCQA GVSRSATIVI | 420 |
| AYLMKHTRMT MTDAYKFVKG KRPIISPNLN FMGQLLEFEE DLNNGVTPRI LTPKLMGVET | 480 |
| VV | |

Seq ID NO: 131 DNA sequence
Nucleic Acid Accession #: NM_005409.3
Coding sequence: 94-378 (underlined sequences correspond to start and
stop codons)

| | |
|---|---|
| 1 11 21 31 41 51 | |
| \| \| \| \| \| \| | |
| TTCCTTTCAT GTTCAGCATT TCTACTCCTT CCAAGAAGAG CAGCAAAGCT GAAGTAGCAG | 60 |
| CAACAGCACC AGCAGCAACA GCAAAAAACA AACATGAGTG TGAAGGGCAT GGCTATAGCC | 120 |
| TTGGCTGTGA TATTGTGTGC TACAGTTGTT CAAGGCTTCC CCATGTTCAA AAGAGGACGC | 180 |
| TGTCTTTGCA TAGGCCCTGG GGTAAAAGCA GTGAAAGTGG CAGATATTGA GAAAGCCTCC | 240 |
| ATAATGTACC CAAGTAACAA CTGTGACAAA ATAGAAGTGA TTATTACCCT GAAAGAAAAT | 300 |
| AAAGGACAAC GATGCCTAAA TCCCAAATCG AAGCAAGCAA GGCTTATAAT CAAAAAAGTT | 360 |
| GAAAGAAAGA ATTTTTAAAA ATATCAAAAC ATATGAAGTC CTGGAAAAGG GCATCTGAAA | 420 |
| AACCTAGAAC AAGTTTAACT GTGACTACTG AAATGACAAG AATTCTACAG TAGGAAACTG | 480 |
| AGACTTTTCT ATGGTTTTGT GACTTTCAAC TTTTGTACAG TTATGTGAAG GATGAAAGGT | 540 |
| GGGTGAAAGG ACCAAAAACA GAAATACAGT CTTCCTGAAT GAATGACAAT CAGAATTCCA | 600 |
| CTGCCCAAAG GAGTCCAGCA ATTAAATGGA TTTCTAGGAA AAGCTACC1T AAGAAAGGCT | 660 |
| GGTTACCATC GGAGTTTACA AAGTGCTTTC ACGTTCTTAC TTGTTGTATT ATACATTCAT | 720 |
| GCATTTCTAG GCTAGAGAAC CTTCTAGATT TGATGCTTAC AACTATTCTG TTGTGACTAT | 780 |
| GAGAACATTT CTGTCTCTAG AAGTTATCTG TCTGTATTGA TCTTTATGCT ATATTACTAT | 840 |
| CTGTGGTTAC AGTGGAGACA TTGACATTAT TACTGGAGTC AAGCCCTTAT AAGTCAAAAG | 900 |
| CATCTATGTG TCGTAAAGCA TTCCTCAAAC ATTTITTCAT GCAAATACAC ACTTCTTTCC | 960 |
| CCAAATATCA TGTAGCACAT CAATATGTAG GGAAACATTC TTATGCATCA TTTGGTTTTT | 1020 |
| TTTATAACCA ATTCATTAAA TGTAATTCAT AAAATGTACT ATGAAAAAAA TTATACGCTA | 1080 |
| TGGGATACTG GCAACAGTGC ACATATTTCA TAACCAAATT AGCAGCACCG GTCTTAATTT | 1140 |
| GATG1TITTC AACTTTTATT CATTGAGATG TTTTGAAGCA ATTAGGATAT GTGTGTTTAC | 1200 |
| TGTACTTTTT GTTTTGATCC GT1TGTATAA ATGATAGCAA TATCTTGGAC ACATTTGAAA | 1260 |
| TACAAAATGT TTTTGTCTAC CAAAGAAAAA TGTTGAAAAA TAAGCAAATG TATACCTAGC | 1320 |
| AATCACTTTT ACTTTTTGTA ATTCTGTCTC TTAGAAAAAT ACATAATCTA ATCAATTTCT | 1380 |
| TTGTTCATGC CTATATACTG TAAAATTTAG GTATACTCAA GACTAGTTTA AAGAATCAAA | 1440 |
| GTCATTTTTT TCTCTAATAA ACTACCACAA CCTTTCTTTT TTAAAAAAAA AAA | |

Seq ID NO: 132 Protein sequence:
Protein Accession #: NP_005400.1

| | |
|---|---|
| 1 11 21 31 41 51 | |
| \| \| \| \| \| \| | |
| MSVKGMAIAL AVILCATVVQ GFPMFKRGRC LCIGPGVKAV EVADIEKASI MYPSNNCDKI | 60 |
| EVIITLKENX GQRCLNPKSK QARLIIKKVE RKNF | |

Seq ID NO: 133 DNA sequence
Nucleic Acid Accession #: NM_012342
Coding sequence: 373-1155 (underlined sequences correspond to start
and stop codons)

| | |
|---|---|
| 1 11 21 31 41 51 | |
| \| \| \| \| \| \| | |
| CTGGCGCGGG CGGGAGCTGC GGCGGATACC CTTGCGTGCT GTGGAGACCC TACTCTCTTC | 60 |
| GCTGAGAACG GCCGCTAGCG GGGACTGAAG GCCGGGAGCC CACTCCCGAC CCGGGGCTAG | 120 |
| CGTGCGTCCC TAGAGTCGAG CGGGGCAAGG GAGCCAGTGG CCGCCGACGG GGGACCGGGA | 180 |
| AACTTTTCTG GGCTCCTGGA GAGCCCTGTA GCCGCGCTCC ATGCTCCGGC AGCGGCCCGA | 240 |
| AACCCAGCCC CGCCGCTGAC GGAGCCCGCC GCTCCGGGCA GGGCCCATGC CCTGCGCGCT | 300 |
| CCGGGGGTCG TAGCTGCCGC CGAGCCGGGG CTCCGGAAGC CGGCGGGGGC GCCGCGGCCG | 360 |
| TGCGGGGCGT CAATGGATCG CCACTCCAGC TACATCTTCA TCTGGCTGCA GCTGGAGCTC | 420 |
| TGCGCCATGG CCGTGCTGCT CACCAAAGGT GAAATTCGAT GCTACTGTGA TGCTGCCCAC | 480 |
| TGTGTAGCCA CTGGTTATAT GTGTAAATCT GAGCTCAGCG CCTGCTTCTC TAGACTTCTT | 540 |
| GATCCTCAGA ACTCAAATTC CCCACTCACC CATGGCTGCC TGGACTCTCT TGCAAGCACG | 600 |
| ACAGACATCT GCCAAGCCAA ACAGGCCCGA AACCACTCTG GCACCACCAT ACCCACATTG | 660 |
| GAATGCTGTC ATGAAGACAT GTGCAATTAC AGAGGGCTGC ACGATGTTCT CTCTCCTCCC | 720 |
| AGGGGTGAGG CCTCAGGACA AGGAAACAGG TATCAGCATG ATGGTAGCAG AAACCTTATC | 780 |
| ACCAAGGTGC AGGAGCTGAC TTCTTCCAAA GAGTTGTGGT TCCGGGCAGC GGTCATTGCC | 840 |
| GTGCCCATTG CTGGAGGGCT GATTITAGTG TTGCTTATTA TGTTGGCCCT GAGGATGCTT | 900 |
| CGAAGTGAAA ATAAGAGGCT GCAGGATCAG CGGCAACAGA TGCTCTCCCG TTTGCACTAC | 960 |
| AGCTTTCACG GACACCATTC CAAAAAGGGG CAGGTTGCAA AGTTAGACTT GGAATGCATG | 1020 |
| GTGCCGGTCA GTGGGCACGA GAACTGCTGT CTGACCTGTG ATAAAATGAG ACAAGCAGAC | 1080 |
| CTCAGCAACG ATAAGATCCT CTCGCTTGTT CACTGGGGCA TGTACAGTGG GCACGGGAAG | 1140 |
| CTGGAATTCG TATGACGGAG TCTTATCTGA ACTACACTTA CTGAACAGCT TGAAGGCCTT | 1200 |
| TTGAGTTCTG CTGGACAGGA GCACTTTATC TGAAGACAAA CTCATTTAAT CATCTTTGAG | 1260 |
| AGACAAAATG ACCTCTGCAA ACAGAATCTT GGATATTTCT TCTGAAGGAT TAITTGCACA | 1320 |
| GACTTAAATA CAGTTAAATG TGTTATTTGC TTTTAAAATT ATAAAAAGCA AAGAGAAGAC | 1380 |
| TTTGTACACA CTGTCACCAG GGTTATTTGC ATCCAAGGGA GCTGGAATTG AGTACCTAAA | 1440 |
| TAAACAAAAA TGTGCCCTAT GTAAGCTTCT ACATCTTGAT TTATTGTAAA GATTTAAAAG | 1500 |
| AAATATATAT ATTTTGTCTG A | |

Seq ID NO: 134 Protein sequence:
Protein Accession #: NP_036474.1

| | |
|---|---|
| 1 11 21 31 41 51 | |
| \| \| \| \| \| \| | |
| MDRHSSYTFI WLQLELCAMA VLLTKGEIRC YCDAAHCVAT GYMCKSELSA CFSRLLDPQN | 60 |
| SNSPLTHGCL DSLASTTDIC QAKQARNHSG TTIPTLECCH EDMCNYRGLH DVLSPPRGEA | 120 |
| SGQGNRYQHD GSRNLITKVQ ELTSSKELWF RAAVIAVPIA GGLILVLLIM LALRMLRSEN | 180 |
| KRLQDQRQQM LSRLHYSFHG HHSKKGQVAK LDLECMVPVS GHENCCLTCD KMRQADLSND | 240 |
| KILSLVHWGM YSGHGKLEFV | |

Seq ID NO: 135 DNA sequence
Nucleic Acid Accession #: NM_001627.1
Coding sequence: 64-1815 (underlined sequences correspond to start and
stop codons)

| | |
|---|---|
| 1 11 21 31 41 51 | |
| \| \| \| \| \| \| | |
| CGGGACGACG CCCCCTCCTG CGGCGTGGAC TCCGTCAGTG GCCCACCAAG AAGGAGGAGG | 60 |
| AATATGGAAT CCAAGGGGGC CAGTTCCTGC CGTCTGCTCT TCTGCCTCTT GATCTCCGCC | 120 |
| ACCGTCTTCA GGCCAGGCCT TGGATGGTAT ACTGTAAATT CAGCATATGG AGATACCATT | 180 |
| ATCATACCTT GCCGACTTGA CGTACCTCAG AATCTCATGT TTGGCAAATG GAAATATGAA | 240 |
| AAGCCCGATG GCTCCCCAGT ATTTATTGCC TTCAGATCCT CTACAAAGAA AAGTGTGCAG | 300 |
| TACGACGATG TACCAGAATA CAAAGACAGA TTGAACCTCT CAGAAAACTA CACTTTGTCT | 360 |
| ATCAGTAATG CAAGGATCAG TGATGAAAAG AGATTTGTGT GCATGCTAGT AACTGAGGAC | 420 |
| AACGTGTTTG AGGCACCTAC AATAGTCAAG GTGTTCAAGC AACCATCTAA ACCTGAAATT | 480 |
| GTAAGCAAAG CACTGTTTCT CGAAACAGAG CAGCTAAAAA AGTTGGGTGA CTGCATTTCA | 540 |
| GAAGACAGTT ATCCAGATGG CAATATCACA TGGTACAGGA ATGGAAAAGT GCTACATCCC | 600 |
| CTTGAAGGAG CGGTGGTCAT AATTTTTAAA AAGGAAATGG ACCCAGTGAC TCAGCTCTAT | 660 |
| ACCATGACTT CCACCCTGGA GTACAAGACA ACCAAGGCTG ACATACAAAT GCCATTCACC | 720 |
| TGCTCGGTGA CATATTATGG ACCATCTGGC CAGAAAACAA TTCATTCTGA ACAGGCAGTA | 780 |
| TTTGATATTT ACTATCCTAC AGAGCAGGTG ACAATACAAG TGCTGCCACC AAAAAATGCC | 840 |
| ATCAAAGAAG GGGATAACAT CACTCTTAAA TGCTTAGGGA ATTGCAACCC TCCCCCAGAG | 900 |
| GAATTTTTGT TTTACTTACC AGGACAGCCC GAAGGAATAA GAAGCTCAAA TACTTACACA | 960 |
| CTGATGGATG TGAGGCGCAA TGCAACAGGA GACTACAAGT GTTCCCTGAT AGACAAAAAA | 1020 |
| AGCATGATTG CTTCAACAGC CATCACAGTT CACTATTTGG ATTfGTCCTT AAACCCAAGT | 1080 |
| GGAGAAGTGA CTAGACAGAT TGGTGATGCC CTACCCGTGT CATGCACAAT ATCTGCTAGC | 1140 |
| AGGAATGCAA CTGTGGTATG GATGAAAGAT AACATCAGGC TTCGATCTAG CCCGTCATTT | 1200 |
| TCTAGTC1TC ATTATCAGGA TGCTGGAAAC TATGTCTGCG AAACTGCTCT GCAGGAGGTT | 1260 |
| GAAGGACTAA AGAAAAGAGA GTCATTGACT CTCATTGTAG AAGGCAAACC TCAAATAAAA | 1320 |
| ATGACAAAGA AAACTGATCC CAGTGGACTA TCTAAAACAA TAATCTGCCA TGTGGAAGGT | 1380 |
| TTTCCAAACC CAGCCATTCA GTGGACAATT ACTGGCAGTG GAAGCGTCAT AAACCAAACA | 1440 |
| GAGGAATCTC CTTATATTAA TGGCAGGTAT TATAGTAAAA TTATCATTTC CCCTGAAGAG | 1500 |
| AATGTTACAT TAACTTGCAC AGCAGAAAAC CAACTGGAGA GAACAGTAAA CTCCTTGAAT | 1560 |
| GTCTCTGCTA TAAGTATTCC AGAACACGAT GAGGCAGACG AGATAAGTGA TGAAAACAGA | 1620 |
| GAAAAGGTGA ATGACCAGGC AAAACTAATT GTGGGAATCG TTGTTGGTCT CCTCCTTGCT | 1680 |
| GCCCTTGTTG CTGGTGTCGT CTACTGGCTG TACATGAAGA AGTCAAAGAC TGCATCAAAA | 1740 |
| CATGTAAACA AGGACCTCGG TAATATGGAA GAAAACAAAA AGTTAGAAGA AAACAATCAC | 1800 |
| AAAACTGAAG CCTAAGAGAG AAACTGTCCT AGTTGTCCAG AGATAAAAAT CATATAGACC | 1860 |
| AATTGAAGCA TGAACGTGGA TTGTATTTAA GACATAAACA AAGACATTGA CAGCAATTCA | 1920 |
| TGGTTCAAGT AITAAGCAGT TCATTCTACC AAGCTGTCAC AGGTTTTCAG AGAATTATCT | 1980 |
| CAAGTAAAAC AAATGAAATT TAATTACAAA CAATAAGAAC AAGTTTTGGC AGCCATGATA | 2040 |
| ATAGGTCATA TGTITGTGTT GGTTCAATTT TTTTTCCGTA AATGTCTGCA CTGAGGATTT | 2100 |
| CTTTTTGGTT TGCCTTTTAT GTAAATTTTT TACGTAGCTA TTTTTATACA CTGTAAGCTT | 2160 |
| TGTTCTGGGA GTTGCTGTTA ATCTGATGTA TAATGTAATG TTTTTATTTC AATTGTTTAT | 2220 |
| ATGGATAATC TGAGCAGGTA CATTTCTGAT TCTGATTGCT ATCAGCAATG CCCCAAACTT | 2280 |
| TCTCATAAGC ACCTAAAACC CAAAGGTGGC AGCTTGTGAA GATTGGGGAC ACTCATATTG | 2340 |
| CCCTAATTAA AAACTGTGAT TTTTATCACA AGGGAGGGGA GGCCGAGAGT CAGACTGATA | 2400 |
| GACACCATAG GAGCCGACTC TTTGATATGC CACCAGCGAA CTCTCAGAAA TAAATCACAG | 2460 |
| ATGCATATAG ACACACATAC ATAATGGTAC TCCCAAACTG ACAATTTTAC CTATTCTGAA | 2520 |
| AAAGACATAA AACAGAATT | |

Seq ID NO: 136 Protein sequence:
Protein Accession #: NP_001618.1

| | |
|---|---|
| 1 11 21 31 41 51 | |
| \| \| \| \| \| \| | |
| MESKGASSCR LLFCLLISAT VFRPGLGWYT VNSAYGDTII IPCRLDVPQN LMFGKWKYEK | 60 |
| PDGSPVFIAF RSSTKKSVQY DDVPEYKDRL NLSENYTLSI SNAPJSDEKR PVCMLVTEDN | 120 |
| VFEAPTIVKV FKQPSKPEIV SKALFLETEQ LKKLGDCISE DSYPDGNITW YRNGKVLHPL | 180 |
| EGAVVIIFKK EMDPVTQLYT MTSTLEYKTT KADIQMPFTC SVTYYGPSGQ KTIHSEQAVF | 240 |
| D1YYPTEQVT IQVLPPKNAI KEGDNITLKC LGNGNPPPEE FLFYLPGQPE GIRSSNTYTL | 300 |
| MDVRRNATGD YKCSLIDKKS MIASTAITVH YLDLSLNFSG EVTRQIGDAL PVSCTISASR | 360 |
| NATVVWMCDN IRLRSSPSFS SLHYQDAGNY VCETALQEVE GLKKRESLTL IVEGKPQIKM | 420 |
| TKKTDPSGLS KTIICHVEGF PKPAIQWTIT GSGSVINQTE ESPYINGRYY SKIIISPEEN | 480 |
| VTLTCTAENQ LERTVNSLNV SAISIPEHDE ADEISDENRE KVNDQAKLIV GIVVGLLLAA | 540 |
| LVAGVVYWLY MKKSKTASKH VNKDLGNMEE NKKLEENNHK TEA | |

Seq ID NO: 137 DNA sequence
Nucleic Acid Accession #: XM_030559
Coding sequence: 1-119 (underlined sequences correspond to start and
stop codons)

| | |
|---|---|
| 1 11 21 31 41 51 | |
| \| \| \| \| \| \| | |
| ATGAACCGCA GCCACCGGCA CGGGGCGGGC AGCGGCTGCC TGGGCACTAT GGAGGTGAAG | 60 |
| AGCAAGTTTG GAGCTGAATT TCGTCGGTTT TCGCTGGAAA GATCAAAACC TGGAAAATTT | 120 |
| GAGGAGTTTT ATGGATTACT ACAACATGTT CATAAGATCC CCAATGTTGA CGTTTTGGTA | 180 |
| GGCTATGCAG ACATCCATGG AGACTTACTA CCTATAAATA ATGATGATAA TTATCACAAA | 240 |
| GCTGTTTCAA CGGCCAATCC ACTGCTTAGG ATATTTATAC AAAAGAAGGA AGAAGCAGAC | 300 |
| TACAGTGCCT TTGGTACAGA CACGCTAATA AAGAAGAAGA ATGTTTTAAC CAACGTATTG | 360 |
| CGTCCTGACA ACCATAGAAA AAAGCCACAT ATAGTCATTA GTATGCCCCA AGACTTTAGA | 420 |
| CCTGTGTCTT CTATTATAGA CGTGGATATT CTCCCAGAAA CGCATCGTAG GGTACGTCTT | 480 |
| TACAAATACG GCACGGAGAA ACCCCTAGGA TTCTACATCC GGGATGGCTC CAGTGTCAGG | 540 |
| GTAACACCAC ATGGCTTAGA AAAGGTTCCA GGGATCTTTA TATCCAGGCT TGTCCCAGGA | 600 |
| GGTCTGGCTC AAAGTACAGG ACTATTAGCT GTTAATGATT AAGTTTTAGA AGTTAATGGC | 660 |
| ATAGAAGTTT CAGGGAAGAG CCTTGATCAA GTAACAGACA TGATGATTGC AAATAGCCGT | 720 |
| AACCTCATCA TAACAGTGAG ACCGGCAAAC CAGAGGAATA ATGTTGTGAG GAACAGTCGG | 780 |
| ACTTCTGGCA GTTCCGGTCA GTCTACTGAT AACAGCCTTC TTGGCTACCC ACAGCAGATT | 840 |
| GAACCAAGCT TTGAGCCAGA GGATGAAGAC AGCGAAGAAG ATGACATTAT CATTGAAGAC | 900 |
| AATTGAGTGC CACAGGAGAT TCCAAAAGCT GTTCCTAATA CTGAGAGCCT GGAGTCATTA | 960 |
| ACACAGATAG AGCTAAGCTT TGAGTCTGGA CAGAATGGCT TTATTCCCTC TAATGAAGTG | 1020 |
| AGCTTAGCAG CCATAGCAAG CAGCTCAAAC ACGGAATTTG AAACACATGC TCCAGATCAA | 1080 |
| AAACTCTTAG AAGAAGATGG AACAATCATA ACATTATGA | |

Seq ID NO: 138 Protein sequence:
Protein Accession #: XP_030559

| | |
|---|---|
| 1 11 21 31 41 51 | |
| \| \| \| \| \| \| | |
| MNRSHRHGAG SGCLGTMEVK SKFGAEFRRF SLERSKPGKF EEFYGLLQHV HKIPNVDVLV | 60 |
| GYADIHGDLL PINNDDNYHX AVSTANPLLR IFIQKKEEAD YSAFGTDTLI KKKNVLTNVL | 120 |
| RPDNHRKKPH IVISMPQDFR PVSSIIDVDI LPETHRRVRL YKYGTEKPLG EYIRDGSSVR | 180 |
| VTPHGLEKVP GIFISRLVPG GLAQSTGLLA VNDEVLEVNG IEVSGKSLDQ VTDMMIANSR | 240 |
| NLIITVRPAN QRNNVVRNSR TSGSSGQSTD NSLLGYPQQI EPSFEPEDED SEEDDILIED | 300 |
| NGVPQQIPKA VPNTESLESL TQIELSFESG QNGFIPSNEV SLAAIASSSN TEFETBAPDQ | 360 |
| KLLEEDGTII TL | |

It is understood that the examples described above in no way serve to limit the true scope of this invention, but rather are presented for illustrative purposes. All publications, sequences of accession numbers, and patent applications cited in this specification are herein incorporated by reference as if each individual publication or patent application were specifically and individually indicated to be incorporated by reference.

What is claimed is:

1. A method of detecting a breast cancer-associated transcript in a cell from a patient, the method comprising contacting a biological sample from the patient with a polynucleotide that selectively hybridizes to a sequence at least 80% identical to a sequence as shown in Tables 1-25.

2. The method of claim 1, wherein the biological sample comprises isolated nucleic acids.

3. The method of claim 2, wherein the nucleic acids are mRNA.

4. The method of claim 2, further comprising the step of amplifying nucleic acids before the step of contacting the biological sample with the polynucleotide.

5. The method of claim 1, wherein the polynucleotide comprises a sequence as shown in Tables 1-25.

6. The method of claim 1, wherein the polynucleotide is immobilized on a solid surface.

7. The method of claim 1, wherein the patient is undergoing a therapeutic regimen to treat breast cancer.

8. The method of claim 1, wherein the patient is suspected of having breast cancer.

9. An isolated nucleic acid molecule consisting of a polynucleotide sequence as shown in Tables 1-25.

10. The nucleic acid molecule of claim 9, which is labeled.

11. An expression vector comprising the nucleic acid of claim 9.

12. A host cell comprising the expression vector of claim 11.

13. An isolated polypeptide which is encoded by a nucleic acid molecule having polynucleotide sequence as shown in Tables 1-25.

14. An antibody that specifically binds a polypeptide of claim 13.

15. The antibody of claim 14, further conjugated to an effector component.

16. The antibody of claim 15, wherein the effector component is a fluorescent label.

17. The antibody of claim 15, wherein the effector component is a radioisotope or a cytotoxic chemical.

18. The antibody of claim 15, which is an antibody fragment.

19. The antibody of claim 15, which is a humanized antibody

20. A method of detecting a breast cancer cell in a biological sample fromi a patient, the method comprising contacting the biological sample with an antibody of claim 14.

21. The method of claim 20, wherein the antibody is further conjugated to an effector component.

22. The method of claim 21, wherein the effector component is a fluorescent label.

23. A method for identifying a compound that modulates a breast cancer-associated polypeptide, the method comprising the steps of:
(i) contacting the compound with a breast cancer-associated polypeptide, the polypeptide encoded by a polynucleotide that selectively hybridizes to a sequence at least 80% identical to a sequence as shown in Tables 1-25; and
(ii) determining the functional effect of the compound upon the polypeptide.

24. A drug screening assay comprising the steps of
(i) administering a test compound to a mammal having breast cancer or a cell isolated therefrom;
(ii) comparing the level of gene expression of a polynucleotide that selectively hybridizes to a sequence at least 80% identical to a sequence as shown in Tables 1-25 in a treated cell or mammal with the level of gene expression of the polynucleotide in a control cell or mammal, wherein a test compound that modulates the level of expression of the polynucleotide is a candidate for the treatment of breast cancer.
